(19) United States
(12) Patent Application Publication
(10) Pub. No.: US 2004/0209897 A1
(43) Pub. Date: Oct. 21, 2004

(54) Mitogen activated protein kinase-activated protein kinase-2 inhibiting compounds

(75) Inventors: William F. Vernier, Oceanside, CA (US); David R. Anderson, Lake St. Louis, MO (US); Dennis P. Phillion, St. Charles, MO (US); Marvin J. Meyers, St. Charles, MO (US); Shridhar G. Hegde, Ballwin, MO (US); David B. Reitz, Chesterfield, MO (US); Ingrid P. Buchler, South University City, MO (US); Matthew W. Mahoney, St. Peters, MO (US); Thomas E. Rogers, Ballwin, MO (US); Gennadiy Poda, Chesterfield, MO (US); Megh Singh, Ellisville, MO (US); Kun K. Wu, Chesterfield, MO (US); Jin Xie, Ballwin, MO (US)

Correspondence Address: Charles E. Dunlap, Nelson Mullins Riley & Scarborough, LLP, 17th Floor, 1320 Main Street, Columbia, SC, 29211, US

(73) Assignee: Pharmacia Corporation, Chesterfield, MO

(21) Appl. No.: 10/742,072

(22) Filed: Dec. 19, 2003

Related U.S. Application Data
(60) Provisional application No. 60/434,962, filed on Dec. 20, 2002.

(51) Int. Cl. A61K 31/519

(52) U.S. Cl. 514/259.2; 514/260.1; 544/255; 544/279

(57) ABSTRACT

Compounds are described which inhibit mitogen activated protein kinase-activated protein kinase-2 (MK-2). Methods of using such compounds for the inhibition of MK-2, and for the prevention or treatment of a disease or disorder that is mediated by TNFα, are described, where the method involves administering to the subject an MK-2 inhibiting compound of the present invention. Therapeutic compositions, pharmaceutical compositions and kits which contain the present MK-2 inhibiting compounds are also described.

### CROSS REFERENCE TO RELATED PATENTS AND PATENT APPLICATIONS

This application is related to and claims the benefit of U.S. Provisional Patent Application Serial No. 60/434,962, filed Dec. 20, 2002, which is incorporated by reference herein in its entirety.

### BACKGROUND OF THE INVENTION

(1) Field of the Invention

The present invention relates to certain cyclic and heterocyclic compounds which inhibit mitogen-activated protein kinase-activated protein kinase-2 (MAPKAP kinase-2, or MK-2), and also to methods of using such compounds to inhibit MK-2 and for the prevention and treatment of TNFα mediated diseases or disorders in subjects that are in need of such prevention and/or treatment.

(2) Description of the Related Art

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif with the sequence Thr-X-Tyr by mitogen-activated protein kinase kinases (MAPKKs). In higher eukaryotes, the physiological role of MAPK signaling has been correlated with cellular events such as proliferation, oncogenesis, development and differentiation. Accordingly, the ability to regulate signal transduction via these pathways could lead to the development of treatments and preventive therapies for human diseases associated with MAPK signaling, such as inflammatory diseases, autoimmune diseases and cancer.

In mammalian cells, three parallel MAPK pathways have been described. The best characterized pathway leads to the activation of the extracellular-signal-regulated kinase (ERK). Less well understood are the signal transduction pathways leading to the activation of the cJun N-terminal kinase (JNK) and the p38 MAPK. See, e.g., Davis, Trends Biochem. Sci. 19:470-473 (1994); Cano, et al., Trends Biochem. Sci. 20:117-122(1995).

The p38 MAPK pathway is potentially activated by a wide variety of stresses and cellular insults. These stresses and cellular insults include heat shock, UV irradiation, inflammatory cytokines (such as TNF and IL-1), tunicamycin, chemotherapeutic drugs (i.e., cisplatinum), anisomycin, sorbitol/hyperosmolarity, gamma irradiation, sodium arsenite, and ischaemia. See, Ono, K., et al, Cellular Signalling 12,1-13 (2000). Activation of the p38 pathway is involved in (1) production of proinflammatory cytokines, such as TNF-α; (2) induction of enzymes, such as Cox-2; (3) expression of an intracellular enzyme, such as iNOS, which plays an important role in the regulation of oxidation; (4) induction of adherent proteins, such as VCAM-1 and many other inflammatory-related molecules. Furthermore, the p38 pathway functions as a regulator in the proliferation and differentiation of cells of the immune system. See, Ono, K., et aL, Id. at 7.

The p38 kinase is an upstream kinase of mitogen-activated protein kinase-activated protein kinase-2 (MAPKAP kinase-2 or MK-2). (See, Freshney, N. W., et al., J. Cell, 78:1039-1049 (1994)). MK-2 is a protein that appears to be predominantly regulated by p38 in cells. Indeed, MK-2 was the first substrate of p38α to be identified. For example, in vitro phosphorylation of MK-2 by p38α activates MK-2. The substrates that MK-2 acts upon, in turn, include heat shock protein 27, lymphocyte-specific protein 1 (LAP1), cAMP response element-binding protein (CREB), ATF1, serum response factor (SRF), and tyrosine hydroxylase. The substrate of MK-2 that has been best characterized is small heat shock protein 27 (hsp27).

The role of the p38 pathway in inflammatory-related diseases has been studied in several animal models. The pyridinyl imidazole compound SB203580 has been shown to be a specific inhibitor of p38 in vivo, and also has been shown to inhibit activation of MK-2, (See, Rouse, J., et al, Cell, 78:1027-1037 (1994); Cuenda, A., et al, Biochem. J., 333:11-15 (1998)), as well as a MAP kinase homologue termed reactivating kinase (RK). (See, Cuenda, A., et al., FEBS Lett., 364(2):229-233 (1995)). Inhibition of p38 by SB203580 can reduce mortality in a murine model of endotoxin-induced shock and inhibit the development of mouse collagen-induced arthritis and rat adjuvant arthritis. See, e.g., Badger, A. M., et al., J. Pharmacol Exp. Ther., 279:1453-1461 (1996). Another p38 inhibitor that has been utilized in an animal model that is believed to be more potent than SB203580 in its inhibitory effect on p38 is SB 220025. A recent animal study has demonstrated that SB 220025 caused a significant dose-dependent decrease in vascular density of granulomas in laboratory rats. (See, Jackson, J. R., et al, J. Pharmacol. Exp. Ther., 284:687-692 (1998)). The results of these animal studies indicated that p38, or the components of the p38 pathway, can be useful therapeutic targets for the prevention or treatment of inflammatory disease.

Due to its integral role in the p38 signaling pathway, MK-2 has been used as a monitor for measuring the level of activation in the pathway. Because of its downstream location in the pathway, relative to p38, MK-2 has been measured as a more convenient, albeit indirect, method of assessing p38 activation. However, so far, research efforts exploring therapeutic strategies associated with the modulation of this pathway have focused mainly on the inhibition of p38 kinase.

Several compounds that inhibit the activity of p38 kinase have been described in U.S. Pat. Nos. 6,046,208, 6,251,914, and 6,335,340. These compounds have been suggested to be useful for the treatment of CSBP/RK/p38 kinase mediated disease. Commercial efforts to apply p38 inhibitors have centered around two p38 inhibitors, the pyridinylimidazole inhibitor SKF 86002, and the 2,4,5 triaryl imidazole inhibitor SB203580. See, Lee, J. C., et al, Immunopharmacology 47, 185-192 (2000). Compounds possessing a similar structure have also been investigated as potential p38 inhibitors. Indeed, p38 MSP kinase's role in various disease states has been elucidated through the use of inhibitors.

Kotlyarov, A. et al, in Nat. Cell Biol., 1(2):94-97-(1999) introduced a targeted mutation into a mouse MK-2 gene, resulting in MK-2-deficient mice. It was shown that mice lacking MK-2 possessed increased stress resistance and survived LPS-induced endotoxic shock better than MK-2⁺ mice. The authors concluded that MK-2 was an essential component in the inflammatory response that regulates biosynthesis of TNFα at a post-transcriptional level. More recently, Lehner, M. D., et al, in J. Immunol., 168(9):4667-4673 (2002), reported that MK-2-deficient mice showed increased susceptibility to Listeria monocytogenes infection, and concluded that MK-2 had an essential role in host defense against intracellular bacteria, probably via regulation of TNF and IFN-gamma production required for activation of antibacterial effector mechanisms.

The location of MK-2 in the p38 signaling pathway at a point that is downstream of p38 offers the potential that MK-2 could act as a focal point for modulating the pathway without affecting as many substrates as would the regulation of an enzyme further upstream in the signaling cascade—such as p38 MAP kinase.

Accordingly, it would be useful to provide compounds and methods that could serve to modulate the activity of MK-2—in particular, to act as inhibitors of MK-2 activity. Such compounds and methods would be useful for the provision of benefits similar to p38 MAP kinase inhibitors, which benefits include the prevention and treatment of diseases and disorders that are mediated by TNFα. It would be even more useful to provide MK-2 inhibitors having improved potency and reduced undesirable side effects, relative to p38 inhibitors.

### SUMMARY OF THE INVENTION

Briefly therefore, the present invention is directed to a novel compound having the structure of formula II: Formula II:

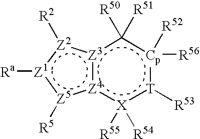

where:

Z¹, Z³ and Z⁴ are independently selected from carbon, and nitrogen;

Z² and Z⁵ are independently selected from carbon, nitrogen, sulfur, and oxygen, and join together with Z¹, Z³ and Z⁴ to form a ring that is selected from a pyrrole, furan, thiophene, oxazole, thiazole, triazole, and imidazole;

when either Z², or Z⁵ is oxygen or sulfur, it has no substituent group;

when Z¹, Z², Z³, Z⁴, and Z⁵ form an imidazole ring, Z¹ is carbon and if Z² and Z⁵ are nitrogen, one is unsubstituted and Z³ and Z⁴ are carbon, if Z³ and Z⁵ are nitrogen, Z⁵ is unsubstituted and Z² and Z⁴ are carbon, and if Z² and Z⁴ are nitrogen, Z² is unsubstituted and Z³ and Z⁵ are carbon;

when Z¹, Z², Z³, Z⁴, and Z⁵ form an oxazole or thiazole ring, Z¹, Z³ and Z⁴ are carbon and one of Z² and Z⁵ is nitrogen that is unsubstituted;

when Z¹, Z², Z³, Z⁴, and Z⁵ form a triazole ring, Z² and Z⁵ are nitrogen that is unsubstituted;

T is selected from C and N;

p is an integer selected from 0,1,2 and 3;

X is selected from C and S;

R^{a} is selected from:

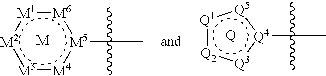

where dashed lines indicate optional single or double bonds;

when ring M is aromatic, M⁵ is carbon and each of M¹, M², M³, M⁴ and M⁶ is independently selected from CR^{b} and N;

when ring M is partially saturated, M⁵ is carbon and each of M¹, M², M³ M⁴ and M⁶ is independently selected from CR^{b}, N, C(R^{b})₂, NR^{b}, oxygen and sulfur;

when ring Q is heteroaromatic, at least one of Q¹, Q², Q³, Q⁴, and Q⁵ is other than carbon, Q⁴ is optionally C or N, and Q¹, Q², Q³, and Q⁵ are each independently selected from CR^{b}, NR^{b} and N; optionally, Q⁴ is C, Q¹ is CR^{b}, and one of Q², Q³, and Q⁵ is optionally oxygen, NR^{b}, or sulfur, and the remainder of Q², Q³, and Q⁵ are independently selected from CR^{b} and N;

when ring Q is partially saturated, Q¹ is optionally CR^{b}, NR^{b}, or N, and Q⁴ is optionally C or N; one of Q², Q³ and Q⁵ is optionally oxygen or sulfur, and the remainder of Q², Q³ and Q⁵ are independently selected from CR^{b}, N, C(R^{b})₂, and NR^{b};

R^{b} is selected from —H, C₁-C₆ alkyl, C₂-C₆ alkenyl, C₂-C₆ alkynyl, C₁-C₆ alkyl-R¹¹, C₂-C₆ alkenyl-R¹¹, C₂-C₆ alkynyl-R¹¹, C₁-C₆ alkyl-(R¹¹)₂, C₂-C₆ alkenyl-(R¹¹)₂, CSR¹¹, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₆ alkyl-NHR⁷, C₁-C₆ alkyl-NR⁸R⁹, (C₁-C₄)alkyl-N(R⁷)—N(R⁸)(R⁹), (C₁-C₄)alkylC(R¹¹)═N—N(R⁸)(R⁹), (C₁-C₄)alkyl-N═N(R⁷), (C₁-C₄)alkyl-N(R⁷)—N═C(R⁸), nitro, cyano, O—R¹³, C₁-C₄ alkyl-OR¹⁰, COR¹¹, SR¹⁰, SSR¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, Si(R¹¹)₃, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹²;

R⁷, R⁸ and R⁹ are each independently selected from —H, C₁-C₆ alkyl, C₂-C₆ alkenyl, C₂-C₆ alkynyl, C₁-C₄ alkyl-R¹¹, C₁-C₆ alkyl-NHR¹³, C₁-C₆ alkyl-NR¹³R¹⁴, O—R¹⁵, C₁-C₄ alkyl-OR¹⁵, CO₂R¹⁵, C(S)OR¹⁵, C(O)SR¹⁵, C(O)R¹⁷, C(S)R¹⁷, CONHR¹⁶, C(S)NHR¹⁶, CON(R¹⁶)₂, C(S)N(R¹⁶)₂, SR¹⁵, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₆-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹⁰ is selected from —H, C₁-C₆ alkyl, C₂-C₆ alkenyl, C₂-C₆ alkynyl, C₁-C₆ alkyl-NHR¹³, C₁-C₆ alkyl-NR¹³R¹⁴, C₁-C₄ alkyl-OR¹⁵, CSR¹¹, CO₂R¹⁵, C(S)OR¹⁵, C(O)SR¹⁵, COR¹⁷, C(S)R¹⁷, CONHR¹⁶, C(S)NHR¹⁶, CON(R¹⁶)₂, C(S)N(R¹⁶)₂, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹¹ is selected from —H, C₁-C₆ alkyl, C₁-C₆ alkoxy, C₂-C₆ alkenyl, C₂-C₆ alkynyl, amino, NHR¹³, NR¹³R¹⁴, N═NR¹³, C₁-C₆ alkyl-NHR¹³, C₁-C₆ alkyl-NR¹³R¹⁴, O—R¹⁵, C₁-C₄ alkyl-OR¹⁵, SR¹⁵, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹² is selected from —H, OH, C₁-C₁₀ alkyl, C₂-C₁₀ alkenyl, C₂-C₁₀ alkynyl, C₁-C₁₀ alkyl-R¹¹, C₂-C₁₀ alkenyl-R¹¹, C₂-C₁₀ alkynyl-R¹¹, C₁-C₁₀ alkyl-(R¹¹)₂, C₂-C₁₀ alkenyl-(R¹¹)₂, CSR¹¹, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₁₀ alkyl-NHR⁷, C₁-C₁₀ alkyl-NR⁸R⁹, (C₁-C₁₀)alkyl-N(R⁷)—N(R⁸)(R⁹), (C₁-C₁₀)alkylC(R¹¹)═N—N(R⁸)(R⁹), (C₁-C₁₀)alkyl-N═N(R⁷), (C₁-C₁₀)alkyl-N(R⁷)—N═C(R⁸), SCN, NCS, C₁-C₁₀ alkyl SCN, C₁-C₁₀ alkyl NCS, nitro, cyano, O—R¹⁰, C₁-C₁₀ alkyl-OR¹⁰, COR¹¹, SR¹⁰, SSR¹⁰, SOR¹¹, SO₂R¹¹, C₁-C₁₀ alkyl-COR¹¹, C₁-C₁₀ alkyl-SR^{1, C}₁-C₁₀ alkyl-SOR¹¹, C₁-C₁₀ alkyl-SO₂R¹¹, halo, Si(R¹¹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹³ and R¹⁴ are each independently selected from —H, C₁-C₆ alkyl, C₂-C₆ alkenyl, C₂-C₆ alkynyl, C₁-C₄ alkyl-R²³, C₁-C₆ alkyl-NHR¹⁹, C₁-C₆ alkyl-NR¹⁹R²⁰, O—R²¹, C₁-C₄ alkyl-OR² , CO₂R²¹, C(S)OR²¹, C(O)SR²¹, C(O)R²³, C(S)R²³, CONHR²², C(S)NHR²², CON(R²²)₂, C(S)N(R²²)₂, SR²¹, SOR²³, SO₂R²³, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁵ and R¹⁶ are independently selected from —H, C₁-C₆ alkyl, C₂-C₆ alkenyl, C₂-C₆ alkynyl, C₁-C₆ alkyl-NHR¹⁹, C₁-C₆ alkyl-NR¹⁹R²⁰, C₁-C₄ alkyl-OR²¹, CSR¹¹, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁷ is selected from —H, C₁-C₆ alkyl, C₂-C₆ alkenyl, C₂-C₆ alkenyl-R¹⁹, C₁-C₆ alkyl-R¹⁹, C₂-C₆ alkynyl, amino, NHR¹⁹, NR¹⁹R²⁰, C₁-C₆ alkyl-NHR¹⁹, C₁-C₆ alkyl-NR¹⁹R²⁰, O—R²¹, C₁-C₄ alkyl-OR²¹, SR²¹, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁸ is selected from —H, OH, C₁-C₁₀ alkyl, C₂-C₁₀ alkenyl, C₂-C₁₀ alkynyl, C₁-C₁₀ alkyl-R²³, C₂-C₁₀ alkenyl-R²³, C₂-C₁₀ alkynyl-R²³, C₁-C₁₀ alkyl-(R²³)₂, C₂-C₁₀ alkenyl-(R²³)₂, CSR²³, amino, NHR¹⁹, NR²⁰R²⁰, N(R¹⁹)—N(R²⁰)(R²⁰), C(R²³)═N—N(R²⁰)(R²⁰), N═N(R¹⁹), N(R¹⁹)—N═C(R²⁰), C(R²³)═N—O(R²¹), ON═C(R²³), C₁-C₁₀ alkyl-NHR¹⁹, C₁-C₁₀ alkyl-NR²⁰R²⁰, (C₁-C₁₀)alkyl-N(R¹⁹)—N(R²⁰)(R²⁰), (C₁-C₁₀)alkylC(R²³)═N—N(R²⁰)(R²⁰), (C₁-C₁₀)alkyl-N═N(R¹⁹), (C₁-C₁₀)alkyl-N(R¹⁹)—N═C(R²⁰), SCN, NCS, C₁-C₁₀ alkyl SCN, C₁-C₁₀ alkyl NCS, nitro, cyano, O—R²¹, C₁-C₁₀ alkyl-OR²¹, COR²³, SR²¹, SSR²¹, SOR²³, SO₂R²³, C₁-C₁₀ alkyl-COR²³, C₁-C₁₀ alkyl-SR²¹, C₁-C₁₀ alkyl-SOR²³, C₁-C₁₀ alkyl-SO₂R²³, halo, Si(R²³)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁹ and R²⁰ are each independently selected from —H, C₁-C₆ alkyl, C₂-C₆ alkenyl, C₂-C₆ alkynyl, C₁-C₄ alkyl-R²⁹, C₁-C₆ alkyl-NHR²⁵, C₁-C₆ alkyl-NR²⁵R²⁶, O—R²⁷, C₁-C₄ alkyl-OR²⁷, CO₂R²⁷, C(S)OR²⁷, C(O)SR²⁷, C(O)R²⁹, C(S)R²⁹, CONHR²⁸, C(S)NHR²⁸, CON(R²⁸)₂, C(S)N(R²⁸)₂, SR²⁷, SOR²⁹, SO₂R²⁹, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C1-C₆ alkyl-CONHR²⁸, C1-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SO₂R²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²¹ and R²² are independently selected from —H, C₁-C₆ alkyl, C₂-C₆ alkenyl, C₂-C₆ alkynyl, C₁-C₆ alkyl-NHR²⁵, C₁-C₆ alkyl-NR²⁵R²⁶, C₁-C₄ alkyl-OR²⁷, CSR¹¹, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²³ is selected from —H, C₁-C₆ alkyl, C₂-C₆ alkenyl, C₂-C₆ alkenyl-R²⁵, C₁-C₆ alkyl-R²⁵, C2-C₆ alkynyl, amino, NHR²⁵, NR²⁵R²⁶, C₁-C₆ alkyl-NHR²⁵, C₁-C₆ alkyl-NR²⁵R²⁶, O—R²⁷, C₁-C₄ alkyl-OR²⁷, SR²⁷, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁴ is selected from —H, OH, C₁-C₁₀ alkyl, C₂-C₁₀ alkenyl, C₂-C₁₀ alkynyl, C₁-C₁₀ alkyl-R²⁹, C₂-C₁₀ alkenyl-R²⁹, C₂-C₁₀ alkynyl-R²⁹, C₁-C₁₀ alkyl-(R²⁹)₂, C₂-C₁₀ alkenyl-(R²⁹)₂, CSR²⁹, N═NR²⁵, amino, NHR²⁵, NR²⁶R²⁶, N(R²⁵)—N(R²⁶)(R²⁶), C(R²⁹)═N—N(R²⁶)(R²⁶), N═N(R²⁵), N(R²⁵)—N═C(R²⁶), C(R²⁹)═N—O(R²⁷), ON═C(R²⁹), C₁-C₁₀ alkyl-NHR²⁵, C₁-C₁₀ alkyl-NR²⁶R²⁶, (C₁-C₁₀)alkyl-N(R²⁵)—N(R²⁶)(R²⁶), (C₁-C₁₀)alkylC(R²⁹)═N—N(R²⁶)(R²⁶), (C₁-C₁₀)alkyl-N═N(R²⁵), (C₁-C₁₀)alkyl-N(R²⁵)—N═C(R²⁶), SCN, NCS, C₁-C₁₀ alkyl SCN, C₁-C₁₀ alkyl NCS, nitro, cyano, O—R²⁷, C₁-C₁₀ alkyl-OR²⁷, COR²⁹, SR²⁷, SSR²⁷, SOR²⁹, SO₂R²⁹, C₁-C₁₀ alkyl-COR²⁹, C₁-C₁₀ alkyl-SR²⁷, C₁-C₁₀ alkyl-SOR²⁹, C₁-C₁₀ alkyl-SO₂R²⁹, halo, Si(R²⁹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁵ and R²⁶ are each independently selected from —H, C₁-C₆ alkyl, C₂-C₆ alkenyl, C₂-C₆ alkynyl, C₁-C₄ alkyl-R³⁵, C₁-C₆ alkyl-NHR³¹, C₁-C₆alkyl-NR³¹R³², O—R³³, C₁-C₄ alkyl-OR³³, CO₂R³³, C(S)OR³³, C(O)SR³³, C(O)R³⁵, C(S)R³⁵, CONHR³⁴, C(S)NHR³⁴, CON(R³⁴)₂, C(S)N(R³⁴)₂, SR³³, SOR³⁵, SO₂R³⁵, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁷ and R²⁸ are independently selected from —H, C₁-C₆ alkyl, C₂-C₆ alkenyl, C₂-C₆ alkynyl, C₁-C₆ alkyl-NHR³¹, C₁-C₆ alkyl-NR³¹R³², C₁-C₄ alkyl-OR³³, CSR¹¹, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁹ is selected from —H, C₁-C₆ alkyl, C₂-C₆ alkenyl, C₂-C₆ alkenyl-R³¹, C₁-C₆ alkyl-R³¹, C2-C₆ alkynyl, amino, NHR³¹, NR³¹R³², C₁-C₆ alkyl-NHR³¹, C₁-C₆ alkyl-NR³¹R³², O—R³³, C₁-C₄ alkyl-OR³³, SR³³, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁰ is selected from —H, OH, C₁-C₁₀ alkyl, C₂-C₁₀ alkenyl, C₂-C₁₀ alkynyl, C₁-C₁₀ alkyl-R³⁵, C₂-C₁₀ alkenyl-R³⁵, C₂-C₁₀ alkynyl-R³⁵, C₁-C₁₀ alkyl-(R³⁵)₂, C₂-C₁₀ alkenyl-(R³⁵)₂, CSR³⁵, amino, NHR³¹, NR³²R³², N(R³¹)—N(R³²)(R³²), C(R³⁵)═N—N(R³²)(R³²), N═N(R³¹), N(R³¹)—N═C(R³²), C(R³⁵)═N—O(R³³), ON═C(R³⁵), C₁-C₁₀ alkyl-NHR³¹, C₁-C₁₀ alkyl-NR³²R³², (C₁-C₁₀)alkyl-N(R³¹)—N(R³²)(R³²), (C₁-C₁₀)alkylC(R³⁵)═N—N(R³²)(R³²), (C₁-C₁₀)alkyl-N═N(R³¹), (C₁-C₁₀)alkyl-N(R³¹)—N═C(R³²), SCN, NCS, C₁-C₁₀ alkyl SCN, C₁-C₁₀ alkyl NCS, nitro, cyano, O—R³³, C₁-C₁₀ alkyl-OR³³, COR³⁵, SR³³, SSR³³, SOR³⁵, SO₂R³⁵, C₁-C₁₀ alkyl-COR³⁵, C₁-C₁₀ alkyl-SR³³, C₁-C₁₀ alkyl-SOR³⁵, C₁-C₁₀ alkyl-SO₂R³⁵, halo, Si(R³⁵)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³¹, R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁶ is selected from —H, alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, and heteroarylalkyl;

R², R⁵, R³⁸, R⁵⁰, R⁵¹, R⁵², R⁵³, and R⁵⁶ are each independently absent, or selected from an R^{b} component; and

R⁵⁴ and R⁵⁵ are each independently oxo, or absent; or

any two of R^{b}, R², R⁵, R⁵⁰, R⁵¹, R⁵², R⁵³, R⁵⁴, and R⁵⁶ optionally join to form a ring of 5, 6, 7, or 8 atoms, where the atoms in the ring are independently selected from M¹, M², M³, M⁴, M⁵, M⁶, Q¹, Q², Q³, Q⁴, Q⁵, Z¹, Z², Z³, Z⁴, Z⁵, CR³⁸, C(R³⁸)₂, C═O, NR⁷, O, S, C═S, S═O, and SO₂.

The present invention is also directed to a novel MK-2 inhibiting compound that is listed in Table I or Table II, below.

The present invention is also directed to a novel method of inhibiting MK-2, the method comprising contacting MK-2 with at least one compound that is described in Table I or Table II, below.

The present invention is also directed to a novel method of preventing or treating a TNFα mediated disease or disorder in a subject, the method comprising administering to the subject an effective amount of an MK-2 inhibiting compound having the structure described in formula II.

The present invention is also directed to a novel method of preventing or treating a TNFα mediated disease or disorder in a subject, the method comprising administering to the subject at least one MK-2 inhibiting compound that is described in Table I or Table II, below.

The present invention is also directed to a novel therapeutic composition comprising a compound having the structure described in formula II.

The present invention is also directed to a novel therapeutic composition comprising at least one MK-2 inhibitory compound that is described in Table I or Table II.

The present invention is also directed to a novel pharmaceutical composition comprising a pharmaceutically acceptable carrier.and at least one MK-2 inhibitory compound having the structure described in formula II.

The present invention is also directed to a novel comprising a dosage form that includes a therapeutically effective amount of at least one MK-2 inhibitory compound having a structure described in formula II.

Among the several advantages found to be achieved by the present invention, therefore, may be noted the provision of a method that could serve to modulate the activity of MK-2—in particular, to inhibit MK-2 activity—and the provision of a method for the prevention and treatment of diseases and disorders that are mediated by TNFα.

### BRIEF DESCRIPTION OF THE DRAWINGS

FIG. 1 is a graph showing paw thickness as a function of time from day 0 to day 7 for MK2 (+/+) and MK2 (−/−) mice, which have received serum injection;

FIG. 2 is a bar chart showing paw thickness at seven days after injection for normal mice, MK2 (+/+) mice receiving serum, MK2 (−/−) mice receiving serum, and MK2 (+/+) mice receiving serum and anti-TNF antibody;

FIG. 3 is a plot of average paw volume for groups of rats receiving no streptococcus cell wall inducement (to induce SCW-induced arthritis) and no treatment (Normal); SCW inducement and treatment only with vehicle (Vehicle); SCW inducement and treatment with vehicle plus 2-{2-[(E)-2-phenylethenyl]pyridin-4-yl}-1,5,6,7-tetrahydro-4H-pyrrolo[3,2-c]pyridin-4-one (Compound “A”) at dosage levels of 200 mpk/day (milligrams/kilogram/day) (A at 200 mpk/day), 60 mpk/day (A at 60 mpk/day), or 20 mpk/day (A at 20 mpk/day); or 2-[2-(2-fluorophenyl)pyridin-4-yl]-1,5,6,7-tetrahydro-4H-pyrrolo[3,2-c]pyridin-4-one trifluoroacetate (Compound “B”) at levels of 240 mpk/day (B at 240 mpk/day), 120 mpk/day (B at 120 mpk/day), or 60 mpk/day (B at 60 mpk/day); and

FIG. 4 is a semi-log plot of percent inhibition in paw swelling as a function of the dosage rate for 2-{2-[(E)-2-phenylethenyl]pyridin-4-yl}-1,5,6,7-tetrahydro-4H-pyrrolo[3,2-c]pyridin-4-one (Compound “A”) and 2-[2-(2-fluorophenyl)pyridin-4-yl]-1,5,6,7-tetrahydro-4H-pyrrolo[3,2-c]pyridin-4-one trifluoroacetate (Compound “B”) , showing typical dose-response behavior for each of the two test compounds.

### DETAILED DESCRIPTION OF THE PREFERRED EMBODIMENTS

In accordance with the present invention, it has been discovered that certain compounds can inhibit the activity of MAPKAP kinase-2. Many of these compounds exhibit their inhibitory effect at low concentrations—having in vitro MK-2 inhibition IC₅₀ values of under 1.0 μM, and with some having IC₅₀ values of under about 0.1 μM, and even as low as about 0.01 μM, or even lower. Accordingly, these compounds can be potent and effective drugs for use in the inhibition of MK-2, and of special value in subjects where such inhibition would be useful. In particular, these compounds would be useful in methods to prevent or treat diseases and disorders that are mediated by TNFα. For example, they can be used for the prevention or treatment of arthritis.

Compounds that have a high degree of MK-2 inhibiting activity offer advantages in therapeutic uses, because therapeutic benefits can be obtained by the administration of lower amounts of the present compounds than with less active compounds. Such highly active compounds also result in fewer side effects, and in some embodiments, demonstrate a selectivity for MK-2 inhibition over the inhibition of other related kinases.

At least one of the present MK-2 inhibitory compounds is an irreversible inhibitor of MK-2. It is believed that in certain instances, irreversible inhibitors have advantages over reversible inhibitors, because they can be used in prolonged suppression of MK-2, limited only by the normal rate of receptor resynthesis, or turnover. An example of an MK-2 inhibitory compound of the present invention that is an irreversible inhibitor of MK-2 is N-[3-[4-(4-oxo-4,5,6,7-tetrahydro-1H-pyrrolo[3,2-c]pyridin-2-yl)pyridin-2-yl]phenyl}acrylamide.

The present MK-2 inhibitory compounds inhibit the activity of the MK-2 enzyme. When it is said that a subject compound inhibits MK-2, it is meant that the MK-2 enzymatic activity is lower in the presence of the compound than it is under the same conditions in the absence of such compound. One method of expressing the potency of a compound as an MK-2 inhibitor is to measure the “IC₅₀” value of the compound. The IC₅₀ value of an MK-2 inhibitor is the concentration of the compound that is required to decrease the MK-2 enzymatic activity by one-half. Accordingly, a compound having a lower IC₅₀ value is considered to be a more potent inhibitor than a compound having a higher IC₅₀ value. As used herein, compounds that inhibit MK-2 can be referred to as MK-2 inhibitors, or MK-2 inhibiting compounds or MK-2 inhibiting agents.

In practice, the selectivity of an MK-2 inhibitor varies depending upon the condition under which the test is performed and on the inhibitors being tested. However, for the purposes of this specification, the selectivity of an MK-2 inhibitor can be measured as a ratio of the in vitro or in vivo IC₅₀ value for inhibition of MK-3, divided by the IC₅₀ value for inhibition of MK-2 (IC_{50 MK-3}/IC_{50 MK-2}). As used herein, the term “IC₅₀” refers to the concentration of a compound that is required to produce 50% inhibition of MK-2 or MK-3 activity. An MK-2 selective inhibitor is any inhibitor for which the ratio of IC_{50 MK-3} to IC_{50 MK-2} is greater than 1. In preferred embodiments, this ratio is greater than 2, more preferably greater than 5, yet more preferably greater than 10, still more preferably greater than 50, and more preferably still, is greater than 100. Such preferred selectivity may indicate an ability to reduce the incidence of side effects incident to the administration of an MK-2 inhibitor to a subject.

Compounds that are useful in the present method include those having the structure shown in formula I: Formula I:

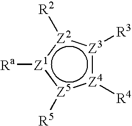

where:

Z¹ is selected from carbon or nitrogen;

Z², Z³, Z⁴, and Z⁵ are independently selected from carbon, nitrogen, sulfur, or oxygen and join to form a pyrrole, furan, thiophene, oxazole, thiazole, isothiazole, triazole, imidazole, oxadiazole, thiadiazole, tetrazole, dithiole, oxathiole, isoxazole, dioxazole, or oxathiazole ring;

when any of Z², Z³, Z⁴, and Z⁵ is oxygen or sulfur, it has no substituent group;

when any of Z², Z³, Z⁴, and Z⁵ is nitrogen or carbon, it is optionally substituted or unsubstituted;

R^{a} is selected from:

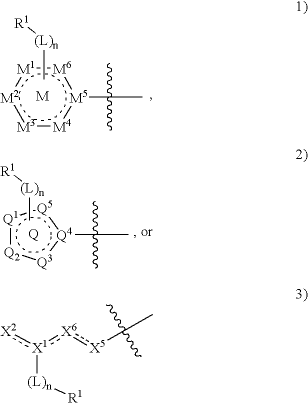

where dashed lines indicate optional single or double bonds;

when ring M is aromatic, M¹ and M⁵ are carbon and each of M², M³, M⁴ and M⁶ is independently selected from CR⁶, or N;

when ring M is partially saturated, M¹ and M⁵ are carbon and each of M², M³ and M⁴ is independently selected from CR⁶, N, C(R⁶)₂, NR⁶, oxygen or sulfur;

when ring Q is aromatic, one of Q¹ and Q⁴ can be carbon or nitrogen, the other is carbon, and Q², Q⁴, and Q⁵ are each independently selected from CR⁶ or N; optionally, Q¹ and Q⁴ are carbon and one of Q², Q³, and Q⁵ is optionally oxygen or sulfur, and the remainder of Q², Q³, and Q⁵ are independently selected from CR⁶ or N;

when ring Q is partially saturated, one of Q¹ and Q⁴ can be nitrogen or carbon, and the other is carbon; one of Q², Q³ and Q⁵ is optionally carbon, oxygen or sulfur, and the remainder of Q², Q³ and Q⁵ are independently selected from CR⁶, N, C(R⁶)₂, or NR⁶;

when R^{a} is structure 3), it is fully conjugated, X² is selected from oxygen or NR⁶, X¹ is carbon, and X⁵ and X⁶ are each independently selected from CR⁶ or N;

R¹, R², R³ R⁴ R⁵, R⁶, R³⁷ and R³⁸ are each independently selected from —H, C₁-C₆ alkyl, C₂-C₆ alkenyl, C₂-C₆ alkynyl, amino, NHR⁷, NR⁸R⁹, NHR⁷-C₁-C₆ alkyl, NR⁸R⁹-C₁-C₆ alkyl, nitro, cyano, O—R¹⁰, C₁-C₄ alkyl-OR¹⁰, aryl, heteroaryl, heterocyclyl, COR¹¹, SR¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;

R⁷, R⁸, are each independently selected from —H, C₁-C₆ alkyl, C₂-C₆ alkenyl, C₂-C₆ alkynyl, amino, NHR¹³, NR¹³R¹⁴, NHR¹³-C₁-C₆ alkyl, NR¹³R¹⁴-C₁-C₆ alkyl, O—R¹⁵, C₁-C₄ alkyl-OR¹⁵, aryl, heteroaryl, heterocyclyl, CO₂R¹⁶, COR¹⁷, CONHR¹⁶, CON(R¹⁶)₂, SR¹⁵, SOR¹⁷, SO₂R¹⁷, C₁-C₁₀ alkyl-CO₂R¹⁶, C₁-C₆ alkyl-COR¹⁷, C₁-C₆ alkyl-CONHR¹⁶, C₁-C₆ alkyl-CON(R¹⁶)₂, C₁-C₆ alkyl-SR¹⁵, C₁-C₆ alkyl-SOR¹⁷, C₁-C₆ alkyl-SO₂R¹⁷, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁸;

R⁹, R¹⁰ are each independently selected from —H, C₁-C₆ alkyl, C₂-C₆ alkenyl, C₂-C₆ alkynyl, NHR¹³-C₁-C₆ alkyl, NR¹³R¹⁴-C₁-C₆ alkyl, C₁-C₄ alkyl-OR¹⁵, aryl, heteroaryl, heterocyclyl, CO₂R¹⁶, COR¹⁷, CONHR¹⁶, CON(R¹⁶)₂, SOR¹⁷, SO₂R¹⁷, C₁-C₆ alkyl-CO₂R¹⁶, C₁-C₆ alkyl-COR¹⁷, C₁-C₆ alkyl-CONHR¹⁶, C₁-C₆ alkyl-CON(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, di-halo C₁-C₄ alkyl, tri-halo C₁-C₄ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁸;

R¹¹ is selected from —H, C₁-C₆ alkyl, C₂-C₆ alkenyl, C₂-C₆ alkynyl, amino, NHR¹³, NR¹³R¹⁴, NHR¹³—C₁-C₆ alkyl, NR¹³R¹⁴—C₁-C₆ alkyl, O—R¹⁵, C₁-C₄ alkyl-OR¹⁵, aryl, heteroaryl, heterocyclyl, SR¹⁵, C₁-C₆ alkyl-CO₂R¹⁶, C₁-C₆ alkyl-COR¹⁷, C₁-C₆ alkyl-CONHR¹⁶, C₁-C₆ alkyl-CON(R¹⁶)₂, C₁-C₆ alkyl-SR¹⁵, C₁-C₆ alkyl-SOR¹⁷, C₁-C₆ alkyl-SO₂R¹⁷, halo, halo C₁-C₄ alkyl di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁸;

R¹² is selected from —H, C₁-C₆ alkyl, C₂-C₆ alkenyl, C₂-C₆ alkynyl, amino, NHR⁷, NR⁸R⁹, NHR⁷—C₁-C₆ alkyl, NR⁸R⁹—C₁-C₆ alkyl, nitro, cyano, O—R¹⁰, C₁-C₄ alkyl-OR¹⁰, aryl, heteroaryl, heterocyclyl, COR¹¹, SR¹⁰, SOR¹¹, SO₂R¹¹, C₁-C₆ alkyl-COR¹¹, C₁-C₆ alkyl-SR^{10, C}₁-C₆ alkyl-SOR¹¹, C₁-C₆ alkyl-SO₂R¹¹, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹⁸;

R¹³ and R¹⁴ are each independently selected from —H, C₁-C₆ alkyl, C₂-C₆ alkenyl, C₂-C₆ alkynyl, amino, NHR¹⁹—C₁-C₆ alkyl, NR¹⁹R²⁰—C₁-C₆ alkyl, C₁-C₄ alkyl-OR²¹, aryl, heteroaryl, heterocyclyl, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁴;

R¹⁵, R¹⁶ are each independently selected from —H, C₁-C₆ alkyl, C₂-C₆ alkenyl, C₂-C₆ alkynyl, NHR¹⁹—C₁-C₆ alkyl, NR¹⁹R²⁰—C₁-C₆ alkyl, C₁-C₄ alkyl-OR²¹, aryl, heteroaryl, heterocyclyl, CO₂R²², COR²³, CONHR²², CON(R²²)₂, SOR²³, SO₂R²⁴, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, di-halo C₁-C₄ alkyl, tri-halo C₁-C₄ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁴;

R¹⁷ is selected from —H, C₁-C₆ alkyl, C₂-C₆ alkenyl, C₂-C₆ alkynyl, amino, NHR¹⁹, NR¹⁹R²⁰, NHR¹⁹—C₁-C₆ alkyl, NR¹⁹R²⁰—C₁-C₆ alkyl, O—R²¹, C₁-C₄ alkyl-OR²¹, aryl, heteroaryl, heterocyclyl, SR²¹, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁴;

R¹⁸ is selected from —H, C₁-C₆ alkyl, C₂-C₆ alkenyl, C₁-C₆ alkynyl, amino, NHR¹⁹, NR¹⁹R²⁰, NHR¹⁹—C₁-C₆ alkyl, NR¹⁹R²⁰—C₁-C₆ alkyl, nitro, cyano, O—R²¹, C₁-C₄ alkyl-OR²¹, aryl, heteroaryl, heterocyclyl, COR²³, SR²¹, SOR²³, SO₂R²³, C₁-C₆ alkyl-COR²³, C₁-C₆ alkyl-SR²¹, C₁-C₆ alkyl-SOR²³, C₁-C₆ alkyl-SO₂R²³, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R²⁴;

R¹⁹ and R²⁰ are each independently selected from —H, C₁-C₆ alkyl, C₂-C₆ alkenyl, C₂-C₆ alkynyl, amino, NHR²⁵—C₁-C₆ alkyl, NR²⁵R²⁶—C₁-C₆ alkyl, C₁-C₄ alkyl-OR²⁷, aryl, heteroaryl, heterocyclyl, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁰;

R²¹ and R²² are each independently selected from —H, C₁-C₆ alkyl, C₂-C₆ alkenyl, C₂-C₆ alkynyl, NHR²⁵—C₁-C₆ alkyl, NR²⁵R²⁶—C₁-C₆ alkyl, C₁-C₄ alkyl-OR²⁷, aryl, heteroaryl, heterocyclyl, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, di-halo C₁-C₄ alkyl, tri-halo C₁-C₄ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁰;

R²³ is selected from —H, C₁-C₆ alkyl, C₂-C₆ alkenyl, C₂-C₆ alkynyl, amino, NHR²⁵, NR²⁵R²⁶, NHR²⁵—C₁-C₆ alkyl, NR²⁵R²⁶—C₁-C₆ alkyl, O—R²⁷, C₁-C₄ alkyl-OR²⁷, aryl, heteroaryl, heterocyclyl, SR²⁷, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁰;

R²⁴ is selected from —H, C₁-C₆ alkyl, C₂-C₆ alkenyl, C₂-C₆ alkynyl, amino, NHR²⁵, NR²⁵R²⁶, NHR²⁵—C₁-C₆ alkyl, NR²⁵R²⁶—C₁-C₆ alkyl, nitro, cyano, O—R²⁷, C₁-C₄ alkyl-OR²⁷, aryl, heteroaryl, heterocyclyl, COR²⁹, SR²⁷, SOR²⁹, SO₂R²⁹, C₁-C₆ alkyl-COR²⁹, C₁-C₆ alkyl-SR²⁷, C₁-C₆ alkyl-SOR²⁹, C₁-C₆ alkyl-SO₂R²⁹, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁰;

R²⁵ and R²⁶ are each independently selected from —H, C₁-C₆ alkyl, C₂-C₆ alkenyl, C₂-C₆ alkynyl, amino, NHR³¹—C₁-C₆ alkyl, NR³¹R³²—C₁-C₆ alkyl, C₁-C₄ alkyl-OR³³, aryl, heteroaryl, heterocyclyl, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR₃₄, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁶;

R²⁷ and R²⁸ are each independently selected from —H, C₁-C₆ alkyl, C₂-C₆ alkenyl, C₂-C₆ alkynyl, NHR³¹—C₁-C₆ alkyl, NR³¹R³²—C₁-C₆ alkyl, C₁-C₄ alkyl-OR³³, aryl, heteroaryl, heterocyclyl, CO₂R³⁴, COR³⁵, CONHR³, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo C₁-C₄ alkyl, di-halo C₁-C₄ alkyl, tri-halo C₁-C₄ alkyl, alkylaryl, alkylheterocyclyl, alkylheteroaryl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁶;

R²⁹ is selected from —H, C₁-C₆ alkyl, C₂-C₆ alkenyl, C₂-C₆ alkynyl, amino, NHR³¹, NR³¹R³², NHR³¹—C₁-C₆alkyl, NR³¹R³²—C₁-C₆alkyl, O—R³³, C₁-C₄ alkyl-OR³³, aryl, heteroaryl, heterocyclyl, SR³³, C₁-C₆ alkyl-CO₂R³⁴, C₁-C₆ alkyl-COR³⁵, C₁-C₆ alkyl-CONHR₃₄, C₁-C₆ alkyl-CON(R³)₂, C₁-C₆ alkyl-SR³³, C₁-C₆ alkyl-SOR³⁵, C₁-C₆ alkyl-SO₂R³⁵, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁶;

R³⁰ is selected from —H, C₁-C₆ alkyl, C₂-C₆ alkenyl, C₂-C₆ alkynyl, amino, NHR³¹, NR³¹R³², NHR³¹—C₁-C₆ alkyl, NR³¹R³²—C₁-C₆ alkyl, nitro, cyano, O—R³³, C₁-C₄ alkyl-OR³³, aryl, heteroaryl, heterocyclyl, COR³⁵, SR³³, SOR³⁵, SO₂R³⁵, C₁-C₆ alkyl-COR³⁵, C₁-C₆ alkyl-SR³³, C₁-C₆ alkyl-SOR³⁵, C₁-C₆ alkyl-SO₂R³⁵, halo, halo C₁-C₄ alkyl, di-halo C₁-C₄ alkyl, tri-halo C₁-C₄ alkyl, alkylaryl, alkylheterocyclyl, alkylheteroaryl, arylalkyl, heteroarylalkyl, heterocyclylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁶;

R³¹, R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁶;

R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R³⁶;

R³⁶ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, heteroarylalkyl;

L is selected from C(R³⁷)₂, O, S, NR³⁷, C═O, C═S, C═C(R³⁷)₂, SO, SO₂, N═NO, CR³⁷═CR³⁷, CR³⁷═N, N═CR³⁷, N═N, NO═N, C═ONR³⁷, C═SR³⁷, NR³⁷C═O, NR³⁷C═S, C═OO, C═OS, C═SO, C═SS, OC═O, SC═O, OC═S, SC═S, S(O)ₘ—(O,S,NR³⁷), (O,S,NR³⁷—S(O)ₘ, C═(O,S)-C═(O,S), aryl, heteroaryl, heterocyclyl, cycloalkyl, alkylaryl, alkylheterocyclyl, alkylheteroaryl, arylalkyl, heterocyclylalkyl, heteroarylalkyl, or C₁-C₁₀ mono- and bicyclic cycloalkyl, wherein aryl, heteroaryl, heterocyclyl, mono- and bicyclic cycloalkyl are optionally substituted with one or more of the groups defined by R¹²;

n is an integer from 0 to 10;

m is an integer from 1 to 2; and

R¹ and R⁶, R⁶ and R², R⁶ and R⁵, R² and R³, R³ and R⁴, R⁶ and R³⁷ or R⁴ and R⁵ optionally join to form a ring of 5, 6, 7, or 8 atoms, where the atoms in the ring are independently selected from M¹, M², M³, M⁴, M⁵, M⁶, Q¹, Q², Q³, Q⁴, Q⁵, X¹, X⁶, X⁵, Z¹, Z², Z³, Z⁴, Z⁵, C(R³⁸)₂, Lₙ, C═O, NR³⁸, O, S, C═S, S═O, or SO₂.

The “M” ring and the “Q” ring of the structure of formula I can have any number of R¹—Lₙ-substituent groups, ranging from zero to one or more per ring atom, and such substituent groups can be located on any atom of the ring having a valence suitable for the addition of a substituent group(s). Each such substituent group can have any number of R¹ groups per L group, ranging from zero to 5. A preferred structure is the presence of either 0 or 1 R¹—Lₙ-substituent groups on the ring. It is also preferred that the R¹—Lₙ-substituent group is attached to the ring at the M¹ or the Q¹ location, respectively.

A preferred embodiment of the compound described in formula I comprises the structure where R³ and R⁴ join to form a six-membered ring having the structure:

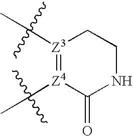

where

Z³ and Z⁴ are carbon.

The meaning of any substituent at any one occurrence in Formula I, or any other general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like. The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical. The term “halo” means halogens such as fluorine, chlorine, and bromine or iodine atoms. The term “haloalkyl” embraces radicals wherein any one or more of the alkyl carbon atoms is substituted with halo as de-fined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical. The term “hydroxyalkyl” embraces linear or branched alkyl radicals having one to about ten carbon atoms any one of which may be substituted with one or more hydroxyl radicals. The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and diaikoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain. The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as:

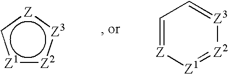

where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures:

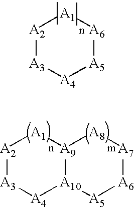

where:

when n=1, m=1 and A₁-A₈ are each CR^{x} or N, A₉ and A₁₀ are carbon;

when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A₉ and A₁₀ are carbon;

when n is greater than or equal to 0, and m is greater than or equal to 0, 1 or more sets of 2 or more adjacent atoms A₁-A₁₀ are sp3O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;

when n is greater than or equal to 0, and m greater than or equal to 0, atoms separated by 2 atoms (i.e., A₁ and A₄) are Sp3O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical. The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃—(CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂(—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl. The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)—atom. The terms “N-alkylamino” and “N,N-dialkylamino” denote amino groups which have been substituted with one alkyl radical and with two alkyl radicals, respectively. The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as:

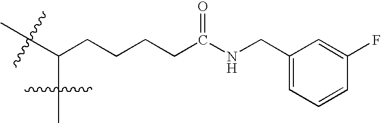

may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if R^{x} is defined as having more than one possible identity.

The present invention also comprises MK-2 inhibiting compounds having the structure shown in formula II: Formula II.

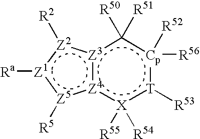

where:

Z¹, Z³ and Z⁴ are independently selected from carbon, and nitrogen;

Z² and Z⁵ are independently selected from carbon, nitrogen, sulfur, and oxygen, and join together with Z¹, Z³ and Z⁴ to form a ring that is selected from a pyrrole, furan, thiophene, oxazole, thiazole, triazole, and imidazole;

when either Z², or Z⁵ is oxygen or sulfur, it has no substituent group;

when Z¹, Z², Z³, Z⁴, and Z⁵ form an imidazole ring, Z¹ is carbon and if Z² and Z⁵ are nitrogen, one is unsubstituted and Z³ and Z⁴ are carbon, if Z³ and Z⁵ are nitrogen, Z⁵ is unsubstituted and Z² and Z⁴ are carbon, and if Z² and Z⁴ are nitrogen, Z² is unsubstituted and Z³ and Z⁵ are carbon;

when Z¹, Z², Z³, Z⁴, and Z⁵ form an oxazole or thiazole ring, Z¹, Z³, and Z⁴ are carbon and one of Z², and Z⁵ is nitrogen that is unsubstituted;

when Z¹, Z², Z³, Z⁴, and Z⁵ form a triazole ring, Z² and Z⁵ are nitrogen that is unsubstituted;

T is selected from C and N;

p is an integer selected from 0,1,2 and 3;

X is selected from C and S;

R^{a} is selected from:

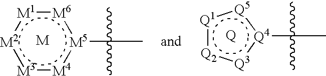

where dashed lines indicate optional single or double bonds;

when ring M is aromatic, M⁵ is carbon and each of M¹, M², M³, M⁴ and M⁶ is independently selected from CR^{b} and N;

when ring M is partially saturated, M⁵ is carbon and each of M¹, M², M³ M⁴ and M⁶ is independently selected from CR^{b}, N, C(R^{b})₂, NR^{b}, oxygen and sulfur;

when ring Q is heteroaromatic, at least one of Q¹, Q², Q³, Q⁴, and Q⁵ is other than carbon, Q⁴ is optionally C or N, and Q¹, Q², Q³, and Q⁵ are each independently selected from CR^{b}, NR^{b} and N; optionally, Q⁴ is C, Q¹ is CR^{b}, and one of Q², Q³, and Q⁵is optionally oxygen, NR^{b}, or sulfur, and the remainder of Q², Q³ and Q⁵ are independently selected from CR^{b} and N;

when ring Q is partially saturated, Q¹ is optionally CR^{b}, NR^{b}, or N, and Q⁴ is optionally C or N; one of Q², Q³ and Q⁵ is optionally oxygen or sulfur, and the remainder of Q², Q³ and Q⁵ are independently selected from CR^{b}, N, C(R^{b})₂, and NR^{b};

R^{b} is selected from —H, C₁-C₆ alkyl, C₂-C₆ alkenyl, C₂-C₆ alkynyl, C₁-C₆ alkyl-R¹¹, C₂-C₆ alkenyl-R¹¹, C₂-C₆ alkynyl-R¹¹, C₁-C₆ alkyl-(R¹¹)₂, C₂-C₆ alkenyl-(R¹¹)₂, CSR¹¹, N═NR⁷, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₆ alkyl-NHR⁷, C₁-C₆ alkyl-NR⁸R⁹, (C₁-C₄)alkyl-N(R⁷)—N(R⁸)(R⁹), (C₁-C₄)alkylC(R¹¹)═N—N(R⁸)(R⁹), (C₁-C₄)alkyl-N═N(R⁷), (C₁-C₄)alkyl-N(R⁷)—N═C(R⁸), nitro, cyano, O—R¹⁰, C₁-C₄ alkyl-OR¹⁰, COR¹¹, SR^{10, SSR}¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, Si(R¹¹)₃, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹²;

R⁷, R⁸ and R⁹ are each independently selected from —H, C₁-C₆ alkyl, C₂-C₆ alkenyl, C₂-C₆ alkynyl, C₁-C₄ alkyl-R¹¹, C₁-C₆ alkyl-NHR¹³, C₁-C₆ alkyl-NR¹³R¹⁴, O—R¹⁵, C₁-C₄ alkyl-OR¹⁵, CO₂R¹⁵, C(S)OR¹⁵, C(O)SR¹⁵, C(O)R¹⁷, C(S)R¹⁷, CONHR¹⁶, C(S)NHR¹⁶, CON(R¹⁶)₂, C(S)N(R¹⁶)₂, SR¹⁵, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹⁰ is selected from —H, C₁-C₆ alkyl, C₂-C₆ alkenyl, C₂-C₆ alkynyl, C₁-C₆ alkyl-NHR¹³, C₁-C₆ alkyl-NR¹³R¹⁴, C₁-C₄ alkyl-OR¹⁵, CSR¹¹, CO₂R¹⁵, C(S)R¹⁵, C(O)SR¹⁵, COR¹⁷, C(S)R¹⁷, CONHR¹⁶, C(S)NHR¹⁶, CON(R¹⁶)₂, C(S)N(R¹⁶)₂, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹¹ is selected from —H, C₁-C₆ alkyl, C₁-C₆ alkoxy, C₂-C₆ alkenyl, C₂-C₆ alkynyl, amino, NHR¹³, NR¹³, R¹⁴, N═NR¹³, C₁-C₆ alkyl-NHR¹³, C₁-C₆ alkyl-NR¹³R¹⁴, O—R¹⁵, C₁-C₄ alkyl-OR¹⁵, SR¹⁵, C₁-C₆ alkyl-CO₂R¹⁵, C₁C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹² is selected from —H, OH, C₁-C₁₀ alkyl, C₂-C₁₀ alkenyl, C₂-C₁₀ alkynyl, C₁-C₁₀ alkyl-R¹¹, C₂-C₁₀ alkenyl-R¹¹, C₂-C₁₀ alkynyl-R¹¹, C₁-C₁₀ alkyl-(R¹¹)₂, C₂-C₁₀ alkenyl-(R¹¹)₂, CSR¹¹, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₁₀ alkyl-NHR⁷, C₁-C₁₀ alkyl-NR⁸R⁹, (C₁-C₁₀)alkyl-N(R⁷)—N(R⁸)(R⁹), (C₁-C₁₀)alkylC(R¹¹)═N—N(R⁸)(R⁹), (C₁-C₁₀)alkyl-N═N(R⁷), (C₁-C₁₀)alkyl-N(R⁷)—N═C(R⁸), SCN, NCS, C₁-C₁₀ alkyl SCN, C₁-C₁₀ alkyl NCS, nitro, cyano, O—R¹⁰, C₁-C₁₀ alkyl-OR¹⁰, COR¹¹, SR¹⁰, SSR¹⁰, SOR¹¹, SO₂R¹¹, C₁-C₁₀ alkyl-COR¹¹, C₁-C₁₀ alkyl-SR¹⁰, C₁-C₁₀ alkyl-SOR¹¹, C₁-C₁₀ alkyl-SO₂R¹¹, halo, Si(R¹¹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹³ and R¹⁴ are each independently selected from —H, C₁-C₆ alkyl, C₂-C₆ alkenyl, C₂-C₆ alkynyl, C₁-C₄ alkyl-R²³, C₁-C₆ alkyl-NHR¹⁹, C₁-C₆ alkyl-NR¹⁹R²⁰, O—R²¹, C₁-C₄ alkyl-OR²¹, CO₂R²¹, C(S)OR²¹, C(O)SR²¹, C(O)R²³, C(S)R²³, CONHR²², C(S)NHR²², CON(R²²)₂, C(S)N(R²²)₂, SR²¹, SOR²³, SO₂R²³, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C1-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁵ and R¹⁶ are independently selected from —H, C₁-C₆ alkyl, C₂-C₆ alkenyl, C₂-C₆ alkynyl, C₁-C₆ alkyl-NHR¹⁹, C₁-C₆ alkyl-NR¹⁹R²⁰, C₁-C₄ alkyl-OR²¹, CSR¹¹, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁷ is selected from —H, C₁-C₆ alkyl, C₂-C₆ alkenyl, C₂-C₆ alkenyl-R¹⁹, C₁-C₆ alkyl-R¹⁹, C₂-C₆ alkynyl, amino, NHR¹⁹, NR¹⁹R²⁰, C₁-C₆ alkyl-NHR¹⁹, C₁-C₆ alkyl-NR¹⁹R²⁰, O—R²¹, C₁-C₄ alkyl-OR²¹, SR²¹, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁸ is selected from —H, OH, C₁-C₁₀ alkyl, C₂-C₁₀ alkenyl, C₂-C₁₀ alkynyl, C₁-C₁₀ alkyl-R²³, C₂-C₁₀ alkenyl-R²³, C₂-C₁₀ alkynyl-R²³, C₁-C₁₀ alkyl-(R²³)₂, C₂-C₁₀ alkenyl-(R²³)₂, CSR²³, amino, NHR¹⁹, NR²⁰R²⁰, N(R¹⁹)—N(R²⁰)(R²⁰), C(R²³)═N—N(R²⁰)(R²⁰), N═N(R¹⁹), N(R¹⁹)—N═C(R²⁰), C(R²³)═N—O(R²¹), ON═C(R²³), C₁-C₁₀ alkyl-NHR¹⁹, C₁-C₁₀ alkyl-NR²⁰R²⁰, (C₁-C₁₀)alkyl-N(R¹⁹)—N(R²⁰)(R²⁰), (C₁-C₁₀)alkylC(R²³)═N—N(R²⁰)(R²⁰), (C₁-C₁₀)alkyl-N═N(R¹⁹), (C₁-C₁₀)alkyl-N(R¹⁹)—N═C(R²⁰), SCN, NCS, C₁-C₁₀ alkyl SCN, C₁-C₁₀ alkyl NCS, nitro, cyano, O—R²¹, C₁-C₁₀ alkyl-OR²¹, COR²³, SR²¹, SSR²¹, SOR²³, SO₂R²³, C₁-C₁₀ alkyl-COR²³, C₁-C₁₀ alkyl-SR²¹, C₁-C₁₀ alkyl-SOR²³, C₁-C₁₀ alkyl-SO₂R²³, halo, Si(R²³)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁹ and R²⁰ are each independently selected from —H, C₁-C₆ alkyl, C₂-C₆ alkenyl, C₂-C₆ alkynyl, C₁-C₄ alkyl-R²⁹, C₁-C₆ alkyl-NHR²⁵, C₁-C₆ alkyl-NR²⁵R²⁶, O—R²⁷, C₁-C₄ alkyl-OR²⁷, CO₂R²⁷, C(S)OR²⁷, C(O)SR²⁷, C(O)R²⁹ (S)R²⁹, CONHR²⁸, C(S)NHR²⁸, CON(R²⁸)₂, C(S)N(R²⁸)₂, SR²⁷, SOR²⁹, SO₂R²⁹, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C1-C₆ alkyl-CONHR²⁸, C1-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²¹ and R²² are independently selected from —H, C₁-C₆ alkyl, C₂-C₆ alkenyl, C₂-C₆ alkynyl, C₁-C₆ alkyl-NHR²⁵, C₁-C₆ alkyl-NR²⁵R²⁶, C₁-C₄ alkyl-OR²⁷, CSR¹¹, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²³ is selected from —H, C₁-C₆ alkyl, C₂-C₆ alkenyl, C₂-C₆ alkenyl-R²⁵, C₁-C₆ alkyl-R²⁵, C2-C₆ alkynyl, amino, NHR²⁵, NR²⁵R²⁶, C₁-C₆ alkyl-NHR²⁵, C₁-C₆ alkyl-NR²⁵R²⁶, O—R²⁷, C₁-C₄ alkyl-OR²⁷, SR²⁷, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁴ is selected from —H, OH, C₁-C₁₀ alkyl, C₂-C₁₀ alkenyl, C₂-C₁₀ alkynyl, C₁-C₁₀ alkyl-R²⁹, C₂-C₁₀ alkenyl-R²⁹, C₂-C₁₀ alkynyl-R²⁹, C₁-C₁₀ alkyl-(R²⁹)₂, C₂-C₁₀ alkenyl-(R²⁹)₂, CSR²⁹, amino, NHR²⁵, NR²⁶R²⁶, N(R²⁵)—N(R²⁶)(R²⁶), C(R²⁹)═N—N(R²⁶)(R²⁶), N═N(R²⁵), N(R²⁵)—N═C(R²⁶), C(R²⁹)═N—O(R²⁷), ON═C(R²⁹), C₁-C₁₀ alkyl-NHR²⁵, C₁-C₁₀ alkyl-NR²⁶R²⁶, (C₁-C₁₀)alkyl-N(R²⁵)—N(R²⁶)(R²⁶), (C₁-C₁₀)alkylC(R²⁹)═N—N(R²⁶)(R²⁶), (C₁-C₁₀)alkyl-N═N(R²⁵), (C₁-C₁₀)alkyl-N(R²⁵)—N═C(R²⁶), SCN, NCS, C₁-C₁₀ alkyl SCN, C₁-C₁₀ alkyl NCS, nitro, cyano, O—R²⁷, C₁-C₁₀ alkyl-OR²⁷, COR²⁹, SR²⁷, SSR²⁷, SOR²⁹, SO₂R²⁹, C₁-C₁₀ alkyl-COR²⁹, C₁-C₁₀ alkyl-SR²⁷, C₁-C₁₀ alkyl-SOR²⁹, C₁-C₁₀ alkyl-SO₂R²⁹, halo, Si(R²⁹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁵ and R²⁶ are each independently selected from —H, C₁-C₆ alkyl, C₂-C₆ alkenyl, C₂-C₆ alkynyl, C₁-C₄ alkyl-R³⁵, C₁-C₆ alkyl-NHR³¹, C₁-C₆ alkyl-NR³¹R³², O—R³³, C₁-C₄ alkyl-OR³³, CO₂R³³, C(S)OR³³, C(O)SR³³, C(O)R³⁵, C(S)R³⁵, CONHR³⁴, C(S)NHR³⁴, CON(R³⁴)₂, C(S)N(R³⁴)₂, SR³³, SOR³⁵, SO₂R³⁵, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR₃₄, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁷ and R²⁸ are independently selected from —H, C₁-C₆ alkyl, C₂-C₆ alkenyl, C₂-C₆ alkynyl, C₁-C₆ alkyl-NHR³¹, C₁-C₆ alkyl-NR³¹R³², C₁-C₄ alkyl-OR³³, CSR¹¹, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁹ is selected from —H, C₁-C₆ alkyl, C₂-C₆ alkenyl, C₂-C₆ alkenyl-R³¹, C₁-C₆ alkyl-R³¹, C₂-C₆ alkynyl, amino, NHR³¹, NR³¹R³², C₁-C₆ alkyl-NHR³¹, C₁-C₆ alkyl-NR³¹R³², O—R³³, C₁-C₄ alkyl-OR³³, SR³³, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁰ is selected from —H, OH, C₁-C₁₀ alkyl, C₂-C₁₀ alkenyl, C₂-C₁₀ alkynyl, C₁-C₁₀ alkyl-R³⁵, C₂-C₁₀ alkenyl-R³⁵, C₂-C₁₀ alkynyl-R³⁵, C₁-C₁₀ alkyl-(R³⁵)₂, C₂-C₁₀ alkenyl-(R³⁵)₂, CSR³⁵, amino, NHR³¹, NR³²R³², N(R³¹)—N(R³²)(R³²), C(R³⁵)═N—N(R³²)(R³²), N═N(R³¹), N(R³¹)—N═C(R³²), C(R³⁵)═N—O(R³³), ON═C(R³⁵), C₁-C₁₀ alkyl-NHR³¹, C₁-C₁₀ alkyl-NR³²R³², (C₁-C₁₀)alkyl-N(R³¹)—N(R³²)(R³²), (C₁-C₁₀)alkylC(R³⁵)═N—N(R³²)(R³²), (C₁-C₁₀)alkyl-N═N(R³¹), (C₁-C₁₀)alkyl-N(R³¹)—N═C(R³²), SCN, NCS, C₁-C₁₀ alkyl SCN, C₁-C₁₀ alkyl NCS, nitro, cyano, O—R³³, C₁-C₁₀ alkyl-OR³³, COR³⁵, SR³³, SSR³³, SOR³⁵, SO₂R³⁵R³⁵, C₁-C₁₀ alkyl-COR³⁵, C₁-C₁₀ alkyl-SR³³, C₁-C₁₀ alkyl-SOR³⁵, C₁-C₁₀ alkyl-SO₂R³⁵, halo, Si(R³⁵)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³¹, R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁶ is selected from —H, alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, and heteroarylalkyl;

R², R⁵, R³⁸, R⁵⁰, R⁵¹, R⁵², R⁵³, and R⁵⁶ are each independently absent, or selected from an R^{b} component; and

R⁵⁴ and R⁵⁵ are each independently oxo, or absent; or

any two of R^{b}, R², R⁵, R⁵⁰, R⁵¹, R⁵², R⁵³, R⁵⁴, and R⁵⁶ optionally join to form a ring of 5, 6, 7, or 8 atoms, where the atoms in the ring are independently selected from M¹, M², M³, M⁴, M⁵, M⁶, Q¹, Q², Q³, Q⁴, Q⁵, Z¹, Z², Z³, Z⁴, Z⁵, CR³⁸, C(R³⁸)₂, C═O, NR⁷, O, S, C═S, S═O, and SO₂.

In a preferred embodiment, the MK-2 inhibiting compound has the structure as shown in formula II, except that when Z² is N and the Z ring is pyrrole, and R^{a} is ring M which is aromatic and in which M² is nitrogen, then R^{b} is other than:

(a) hydrogen, halo, R^{K}, hydroxy-R^{K}—, or R^{K}—O—R^{K}—;

(b) A^{r}—, A^{r}—R^{K}—, A^{r}—O—, A^{r}—S—, A^{r}—NH—, or A^{r}—CO—; and

(c) R^{K}—CO—, R^{K}—O—CO—, or R^{K}—NH—CO—; or two of R^{K} which are attached to adjacent carbon atoms on the pyridine ring complete a fused benzene ring, the benzene ring being optionally substituted with one or two substituents selected from C₁-C₄ alkyl, halo-substituted C₁-C₄ alkyl, halo-substituted C₁-C₄ alkoxy, nitro, hydroxy, amino and halo;

where R^{K} is C₁-C₆ alkyl optionally substituted by up to four halogen atoms; and

A^{r} is selected from phenyl, naphthyl, pyridyl, quinonyl, thienyl, furyl, pyrrolyl, indolyl, benzothienyl and benzofuryl, the aryl or heteroaryl groups being optionally substituted with one or two substituents selected from C₁-C₄ alkyl, C₁-C₄ alkoxy, halo-substituted C₁-C₄ alkyl, halo-substituted C₁-C₄ alkoxy, nitro, hydroxy, amino, R^{K}—NH—, (R^{K})₂N—, halo, formyl, halo-substituted phenoxy, halo-substituted phenyl, C₁-C₄ alkyl-substituted phenoxy, halo-substituted phenylthio, C₁-C₄ alkoxycarbonyl, C₁-C₄ alkylthio, and C₁-C₄ alkyl-SO—.

In an optional embodiment, the ring of 5, 6, 7, or 8 atoms that is optionally formed by the joining of any two of R^{b}, R², R⁵, R⁵⁰, R⁵¹, R⁵², R⁵³, R⁵⁴, and R⁵⁶ where the atoms in the ring are independently selected from M¹, M², M³, M⁴, M⁵, M⁶, Q¹, Q², Q³, Q⁴, Q⁵, Z¹, Z², Z³, Z⁴, Z⁵, CR³⁸, C(R³⁸)₂, C═O, NR⁷, O, S, C═S, S═O, and SO₂, is absent in the compound of formula II.

The present MK-2 inhibiting compound optionally has the structure that is described above for formula II, except wherein:

p is 1;

T is N;

X is C;

R⁵⁴ is oxo; and

R⁵⁵ is absent.

The present MK-2 inhibiting compound optionally has the structure that is described above for formula II, except wherein Z¹, Z², Z³, Z⁴, and Z⁵ form a pyrrole or imidazole ring.

The present MK-2 inhibiting compound optionally has the structure that is described above for formula II, except wherein:

p is 1;

T is N;

X is C;

R⁵⁴ is oxo;

R⁵⁵ is absent; and

Z¹, Z², Z³, Z⁴, and Z⁵ form a pyrrole or imidazole ring.

In a preferred embodiment, Z¹, Z², Z³, Z⁴, and Z⁵ form a pyrrole ring.

In another embodiment, the present MK-2 inhibiting compound optionally has the structure that is described above for formula II, except wherein:

p is 1;

T is N;

X is C;

R⁵⁴ is oxo;

R⁵⁵ is absent;

Z¹, Z², Z³, Z⁴, and Z⁵ form a pyrrole ring; and

R^{a} is

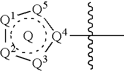

The present MK-2 inhibiting compound optionally has the structure that is described above for formula II, except wherein:

p is 1;

T is N;

X is C;

R⁵⁴ is oxo;

R⁵⁵ is absent;

Z¹, Z², Z³, Z⁴, and Z⁵ form a pyrrole ring; and

R^{a} is

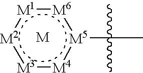

The present MK-2 inhibiting compound optionally has the structure that is described above for formula II, except wherein:

p is 1;

T is N;

X is C;

R⁵⁴ is oxo;

R⁵⁵ is absent;

Z¹, Z², Z³, Z⁴, and Z⁵ form a pyrrole ring;

R^{a} is

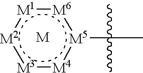

and, wherein the M-ring is selected from pyridine and pyrimidine.

In a preferred embodiment, the M-ring is pyridine.

In another embodiment, the MK-2 inhibiting compound has a structure as described by formula II, except wherein:

p is 1;

T is N;

X is C;

Z¹, Z³, Z⁴, and Z⁵ are carbon;

Z² is nitrogen;

Z¹, Z², Z³, Z⁴ and Z⁵ form a pyrrole ring;

R^{a} is

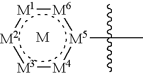

when ring M is aromatic, M² is N, M⁵ is carbon, M¹ is CR^{b}, M³ is CR⁵⁸, M⁴ is CR⁵⁹, and M⁶ is N, or CR⁶⁰;

when ring M is partially saturated, M² is N, M⁵ is carbon, M¹ is CR^{b} or C(R^{b})₂, M³ is CR⁵⁸ or C(R⁵⁸)₂, M⁴ is CR⁵⁹ or C(R⁵⁹)₂, and M⁶ is independently selected from CR⁶⁰, N and C(R⁶⁰)₂;

M¹, M², M³, M⁴, M⁵ and M⁶ join to form a pyridine or pyrimidine ring;

R² is selected from H, and C₁-C₄ alkyl, or optionally is absent;

R⁵ is selected from H, halo, C₁-C₄ alkyl, amino, diazo, nitro, and aryl;

R⁵⁰ and R⁵¹ are each independently selected from H, C₁-C₄ alkyl, and aryl, or one of R⁵⁰ and R⁵¹ is absent;

R⁵² is selected from H, C₁-C₄ alkyl, C₁-C₄ haloalkyl, hydroxy C₁-C₄ alkyl, C₁-C₆ cycloalkyl, aryl, and aryl-C₁-C₄-alkoxy-C₁-C₄-alkyl;

R⁵³ is selected from H, C₁-C₄ alkenylcarboxyl, and C₁-C₄ alkyl;

R⁵⁴ is oxo;

R⁵⁵ is absent;

R⁵⁶ is absent, or is selected from an R⁵² group;

R⁵⁸ is selected from H, halo, amino, aryl-C₁-C₄-cycloalkyl, and haloaryl;

R⁵⁹ is selected from H, and halo, or optionally is absent, or R⁵⁷ and R⁵⁹ optionally join to form a six-membered phenyl ring; and

R⁶⁰ is H.

In another embodiment, the MK-2 inhibiting compound has a structure as described by formula II, except wherein:

p is 1;

T is N;

X is C;

Z¹, Z³, Z⁴, and Z⁵ are carbon;

Z² is nitrogen;

Z¹, Z², Z³, Z⁴ and Z⁵ form a pyrrole ring;

R^{a} is

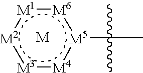

when ring M is aromatic, M² is N, M⁵ is carbon, M¹ is CR^{b}, M³ is CR⁵⁸, M⁴ is CR⁵⁹, and M⁶ is CR⁶⁰;

when ring M is partially saturated, M² is N, M⁵ is carbon, M¹ is CR^{b} or C(R^{b})₂, M³ is CR⁵⁸ or C(R⁵⁸)₂, M⁴ is CR⁵⁹ or C(R⁵⁹)₂, and M⁶ is independently selected from CR⁶⁰, and C(R⁶⁰)₂;

M¹, M², M³, M⁴, M⁵ and M⁶ join to form a pyridine ring;

R² is selected from H, and C₁-C₄ alkyl, or optionally is absent;

R⁵ is selected from H, halo, C₁-C₄ alkyl, amino, diazo, nitro, and aryl;

R⁵⁰ and R⁵¹ are each independently selected from H, C₁-C₄ alkyl, and aryl, or one of R⁵⁰ and R⁵¹ is absent;

R⁵² is selected from H, C₁-C₄ alkyl, C₁-C₄ haloalkyl, hydroxy C₁-C₄ alkyl, C₁-C₆ cycloalkyl, aryl, and aryl-C₁-C₄-alkoxy-C₁-C₄-alkyl;

R⁵³ is selected from H, C₁-C₄ alkenylcarboxyl, and C₁-C₄ alkyl;

R⁵⁴ is oxo;

R⁵⁵ is absent;

R⁵⁶ is absent, or is selected from an R⁵² group;

R⁵⁸ is selected from H, halo, amino, aryl-C₁-C₄-cycloalkyl, and haloaryl;

R⁵⁹ is selected from H, and halo, or optionally is absent, or R⁵⁷ and R⁵⁹ optionally join to form a six-membered phenyl ring; and

R⁶⁰ is H.

Table I shows examples of MK-2 inhibiting compounds of the present invention, and also shows the chemical name and, where available, the IC₅₀ value of the compound for MK-2 inhibition. More examples of MK-2 inhibiting compounds of the present invention are listed in Table II. It is believed that any of the compounds that are listed in Table I and Table II are MK-2 inhibiting compounds that can be used in the method of the present invention. However, neither the novel MK-2 inhibiting compounds, nor the uses of an MK-2 inhibiting compound that are described herein are intended to be limited to the compounds that are presented in the Tables.

TABLE I
Inhibiting compounds; Structure, name and MK-2 inhibiting activity

| | | | |
|---|---|---|---|
| | | | MAPKAP2 |
| | | Avg. IC₅₀ | |
| Number | Structure^{a} | Compound Name(s)^{b} | (uM) |

| | | | | |
|---|---|---|---|---|
| 1 | 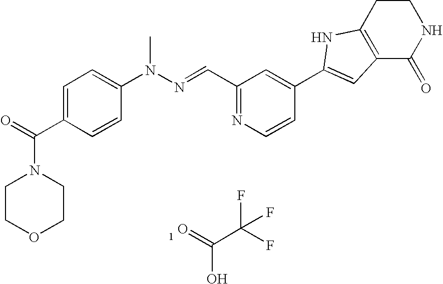 | 4-(4-oxo-4,5,6,7-tetrahydro-1H-pyrrolo[3,2-c]pyridin-2- yl)pyridine-2-carbaldehyde methyl[4-(morpholin-4- ylcarbonyl)phenyl]hydrazone trifluoroacetate | 0.00505 | |
| 2 | 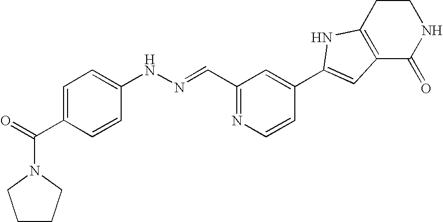 | 4-(4-oxo-4,5,6,7-tetrahydro-1H-pyrrolo[3,2-c]pyridin-2- yl)pyridine-2-carbaldehyde [4-(pyrrolidin-1- ylcarbonyl)phenyl]hydrazone | 0.00535 | |
| 3 | 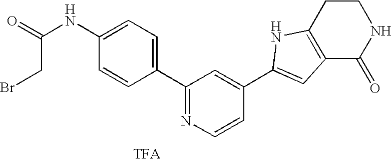 | 2-bromo-N-{4-[4-(4-oxo-4,5,6,7-tetrahydro-1H-pyrrolo[3,2- c]pyridin-2-yl)pyridin-2-yl]phenyl}acetamide trifluoroacetate | 0.00585 | |
| 4 | 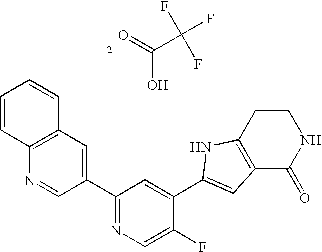 | 2-(5-fluoro-2-quinolin.3-ylpyridin-4-yl)-1,5,6,7-tetrahydro-4H- pyrrolo[3,2-c]pyridin-4-one trifluoroacetate | 0.007 | |
| 5 | 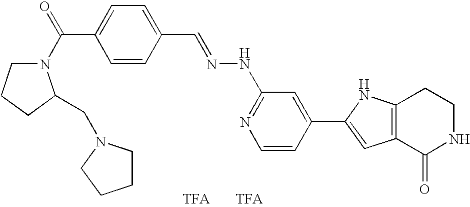 | 4-{[2-(pyrrolidin-1-ylmethyl)pyrrolidin-1- yl]carbonyl}benzaldehyde [4-(4-oxo-4,5,6,7-tetrahydro-1H- pyrrolo[3,2-c]pyridin-2-yl)pyridin-2-yl]hydrazone bis(trifluoroacetate) | 0.0078 | |
| 6 | 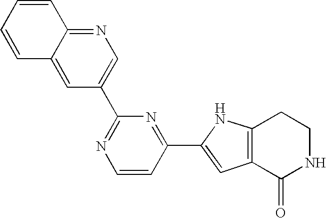 | 2-(2-quinolin-3-ylpyrimidin-4-yl)-1,5,6,7-tetrahydro-4H- pyrrolo[3,2-c]pyridin-4-one | 0.0079 | |
| 7 | 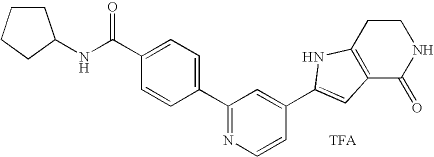 | N-cyclopentyl-4-[4-(4-oxo-4,5,6,7-tetrahydro-1H-pyrrolo[3,2- c]pyridin-2-yl)pyridin-2-yl]benzamide | 0.008 | |
| 8 | 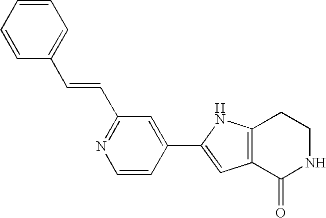 | 2-{2-[(E)-2-phenylethenyl]pyridin-4-yl}-1,5,6,7-tetrahydro-4H- pyrrolo[3,2-c]pyridin-4-one | 0.00805 | |
| 9 | 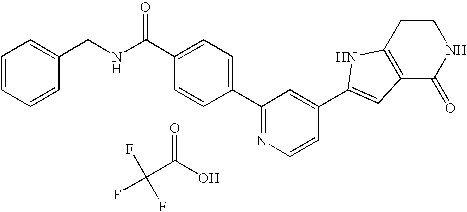 | N-benzyl-4-[4-(4-oxo-4,5,6,7-tetrahydro-1H-pyrrolo[3,2-c]pyridin- 2-yl)pyridin-2-yl]benzamide trifluoroacetate | 0.0084 | |
| 10 | 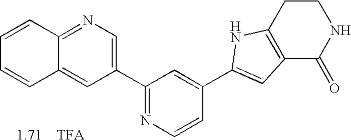 | 2-(2-quinolin-3-ylpyridin-4-yl)-1,5,6,7-tetrahydro-4H-pyrrolo[3,2- c]pyridin-4-one trifluoroacetate | 0.0085 | |
| 11 | 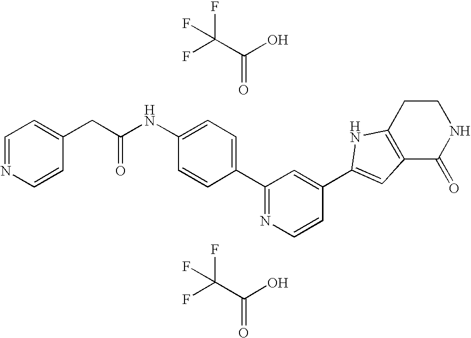 | N-{4-[4-(4-oxo-4,5,6,7-tetrahydro-1H-pyrrolo[3,2-c]pyridin-2- yl)pyridin-2-yl]phenyl}-2-pyridin-4-ylacetamide bis(trifluoroacetate) | 0.0085 | |
| 12 | 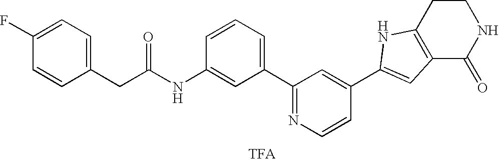 | 2-(4-fluorophenyl)-N-{3-(4-(4-oxo-4,5,6,7-tetrahydro-1H- pyrrolo[3,2-c]pyridin-2-yl)pyridin-2-yl]phenyl}acetamide trifluoroacetate | 0.0085 | |
| 13 | 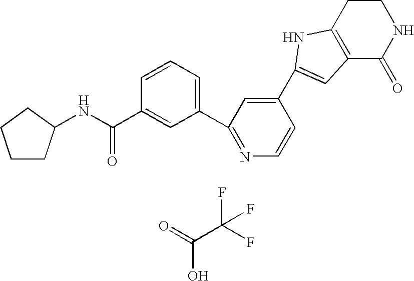 | N-cyclopentyl-3-[4-(4-oxo-4,5,6,7-tetrahydro-1H-pyrrolo[3,2- c]pyridin-2-yl)pyridin-2-yl]benzamide trifluoroacetate | 0.00855 | |
| 14 | 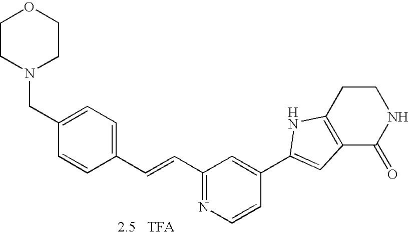 | 2-(2-{(E)-2-[4-(morphoIin-4-ylmethyl)phenyl]vinyl}pyridin-4-yl)- 1,5,6,7-tetrahydro-4H-pyrrolo[3,2-c]pyridin-4-one trifluoroacetate | 0.00855 | |
| 15 | 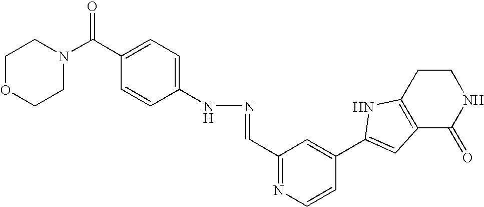 | 4-(4-oxo-4,5,6,7-tetrahydro-1H-pyrrolo[3,2-c]pyridin-2- yl)pyridine-2-carbaldehyde [4-(morpholin-4- ylcarbonyl)phenyl]hydrazone | 0.0087 | |
| 16 | 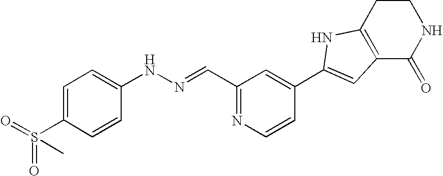 | 4-(4-oxo-4,5,6,7-tetrahydro-1H-pyrrolo[3,2-c]pyridin-2- yl)pyridine-2-carbaldehyde [4-(methylsulfonyl)phenyl]hydrazone | 0.0089 | |
| 17 | 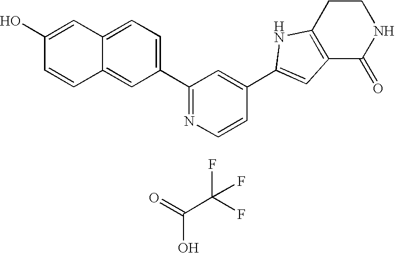 | 2-[2-(6-hydroxy-2-naphthyl)pyridin-4-yl]-1,5,6,7-tetrahydro-4H- pyrrolo[3,2-c]pyridin-4-one trifluoroacetate | 0.0092 | |
| 18 | 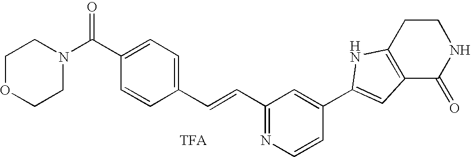 | 2-(2-{(E)-2-[4-(morpholin-4-ylcarbonyl)phenyl]vinyl}pyridin-4-yl)- 1,5,6,7-tetrahydro-4H-pyrrolo[3,2-c]pyridin-4-one trifluoraacetate | 0.00925 | |
| 19 | 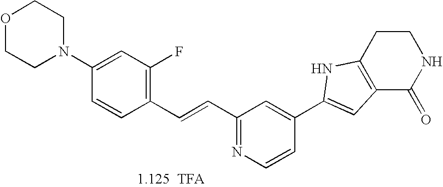 | 2-{2-[(E)-2-(2-fluoro-4-morpholin-4-ylphenyl)vinyl]pyridin-4-yl}- 1,5,6,7-tetrahydro-4H-pyrrolo[3,2-c]pyridin-4-one trifluoroacetate | 0.0094 | |
| 20 | 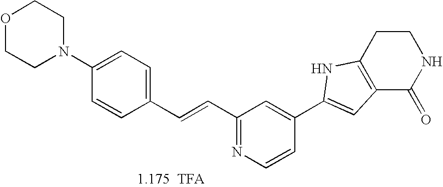 | 2-{2-[(E)-2-(4-morpholin-4-ylphenyl)vinyl]pyridin-4-yl}-1,5,6,7- tetrahydro-4H-pyrrolo[3,2-c]pyridin-4-one trifluoroacetate | 0.00945 | |
| 21 | 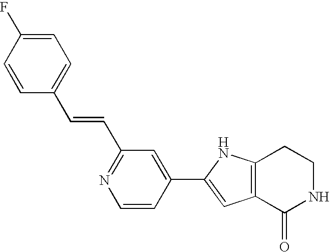 | 2-{2-[(E)-2-(4-fluorophenyl)ethenyl]pyridin-4-yl}-1,5,6,7- tetrahydro-4H-pyrrolo[3,2-c]pyridin-4-one | 0.0095 | |
| 22 | 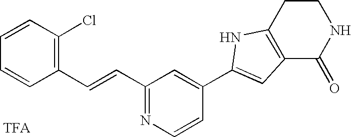 | 2-{2-[(E)-2-(2-chlorophenyl)vinyl]pyridin-4-yl)-1,5,6,7-tetrahydro- 4H-pyrrolo[3,2-c]pyridin-4-one trifluoroacetate | 0.0095 | |
| 23 | 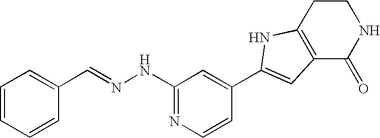 | benzaldehyde [4-(4-oxo-4,5,6,7-tetrahydro-1H-pyrrolo[3,2- c]pyridin-2-yl)pyridin-2-yl]hydrazone | 0.00953 | |
| 24 | 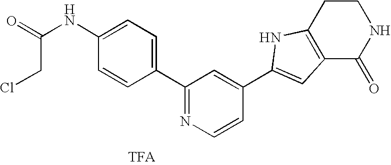 | 2-chloro-N-{4-[4-(4-oxo-4,5,6,7-tetrahydro-1H-pyrrolo[3,2- c]pyridin-2-yl)pyridin-2-yl]phenyl}acetamide trifluoroacetate | 0.00975 | |
| 25 | 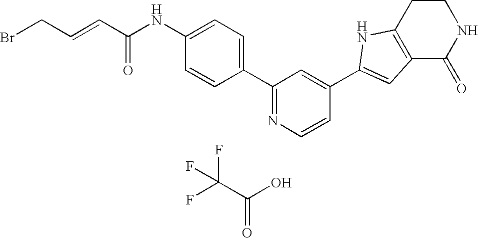 | (2E)-4-bromo-N-{4-[4-(4-oxo-4,5,6,7-tetrahydro-1H-pyrrolo(3,2- c]pyridin-2-yl)pyridin-2-yl]phenyl)but-2-enamide trifluoroacetate | 0.00985 | |
| 26 | 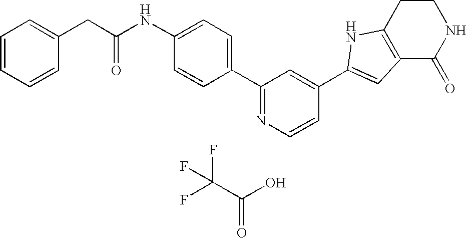 | N-{4-[4-(4-oxo-4,5,6,7-tetrahydro-1H-pyrrolo[3,2-c]pyridin-2- yl)pyridin-2-yl]phenyl}-2-phenylacetamide trifluoroacetate | 0.01 | |
| 27 | 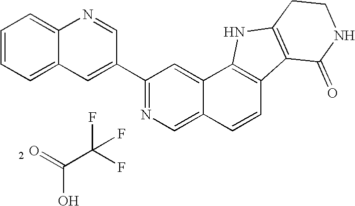 | 2-quinolin-3-yl-8,9,10,11-tetrahydro-7H- pyrido[3′,4′,:4,5]pyrrolo[2,3-f]isoquinolin-7-one trifluoracetate | 0.0101 | |
| 28 | 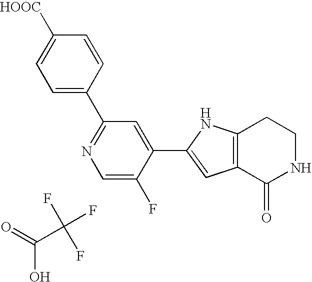 | 4-[5-fluoro-4-(4-oxo-4,5,6,7-tetrahydro-1H-pyrrolo[3,2-c]pyridin- 2-yl)pyridin-2-yl]benzoic acid trifluoroacetate | 0.0101 | |
| 29 | 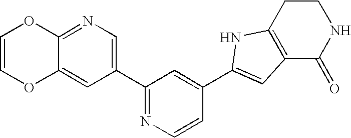 | 2-(2-[1,4]dioxino[2,3-b]pyridin-7-ylpyridin-4-yl)-1,5,6,7- tetrahydro-4H-pyrrolo[3,2-c]pyridin-4-one | 0.0103 | |
| 30 | 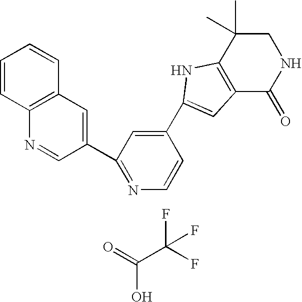 | 7,7-dimethyl-2-(2-quinolin-3-ylpyridin-4-yl)-1,5,6,7-tetrahydro- 4H-pyrrolo[3,2-c]pyridin-4-one trifluoroacetate | 0.0103 | |
| 31 | 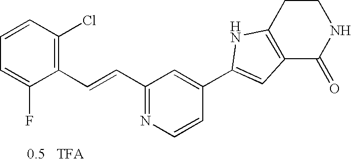 | 2-{2-[(E)-2-(2-chloro-6-fluorophenyl)vinyl]pyridin-4-yl}-1,5,6,7- tetrahydro-4H-pyrrolo[3,2-c]pyridin-4-one trifluoroacetate | 0.0107 | |
| 32 | 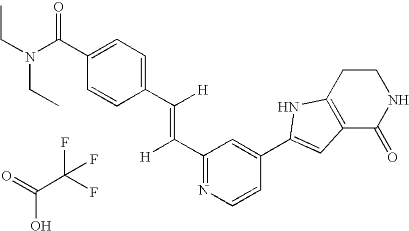 | N,N-diethyl-4-{(E)-2-[4-(4-oxo-4,5,6,7-tetrahydro-1H-pyrrolo[3,2- c]pyridin-2-yl)pyridin-2-yl]vinyl}benzamide trifluoroacetate | 0.0113 | |
| 33 | 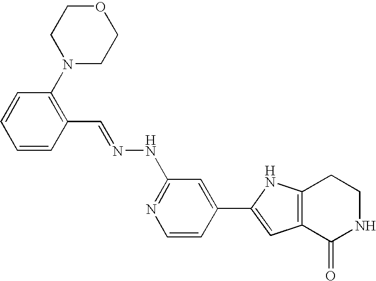 | 2-morpholin-4-ylbenzaldehyde [4-(4-oxo-4,5,6,7-tetrahydro-1H- pyrrolo[3,2-c]pyridin-2-yl)pyridin-2-yl]hydrazone | 0.0114 | |
| 34 | 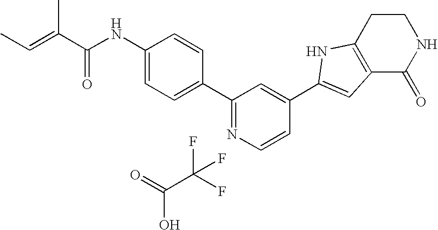 | (2E)-2-methyl-N-{4-[4-(4-oxo-4,5,6,7-tetrahydro-1H-pyrrolo[3,2- c]pyridin-2-yl)pyridin-2-yl]phenyl}but-2-enamide trifluoroacetate | 0.0116 | |
| 35 | 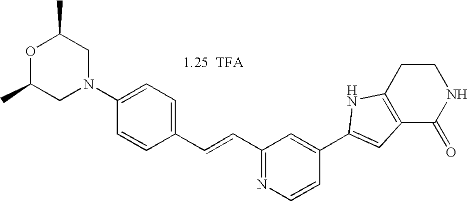 | 2-[2-((E)-2-{4-[(2R,6S)-2,6-dimethylmorpholin-4- yl]phenyl}vinyl)pyridin-4-yl]-1,5,6,7-tetrahydro-4H-pyrrolo[3,2- c]pyridin-4-one trifluoroacetate | 0.0117 | |
| 36 | 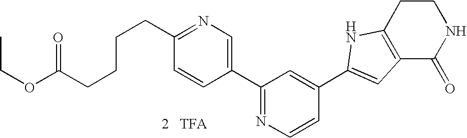 | ethyl 5-[4-(4-axo-4,5,6,7-tetrahydro-1H-pyrrolo[3,2-c]pyridin-2- yl)-2,3′-bipyrldin-6′-yl]pentanoate trifluoroacetate | 0.0121 | |
| 37 | 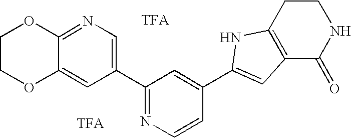 | 2-[2-(2,3-dihydro[1,4]dioxino[2,3-b]pyridin-7-yl)pyridin-4-yl]- 1,5,6,7-tetrahydro-4H-pyrrolo[3,2-c]pyridin-4-one bis(trifluoroacetate) | 0.0125 | |
| 38 | 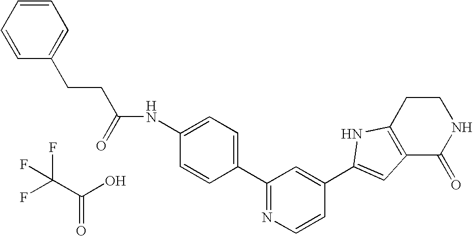 | N-{4-[4-(4-oxo-4,5,6,7-tetrahydro-1H-pyrrolo[3,2-c]pyridin-2- yl)pyridin-2-yl]phenyl}-3-phenylpropanamide trifluoroacetate | 0.0125 | |
| 39 | 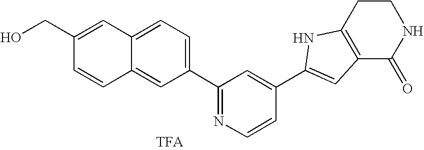 | 2-{2-[6-(hydroxymethyl)-2-naphthyl]pyridin-4-yl}-1,5,6,7- tetrahydro-4H-pyrrolo[3,2-c]pyridin-4-one trifluoroacetate | 0.0126 | |
| 40 | 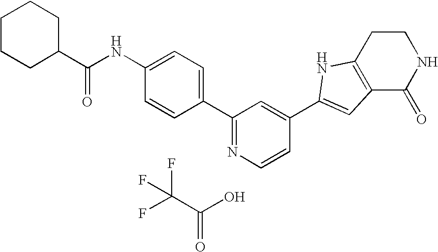 | N-{4-[4-(4-oxo-4,5,6,7-tetrahydro-1H-pyrrolo[3,2-c]pyridin-2- yl)pyridin-2-yl]phenyl}cyclohexanecarboxamide trifluoroacetate | 0.0126 | |
| 41 | 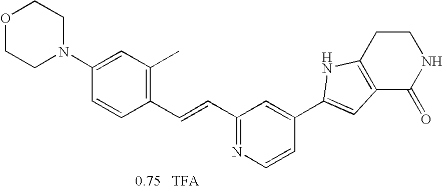 | 2-{2-[(E)-2-(2-methyl-4-morpholin-4-ylphenyl)vinyl]pyridin-4-yl}- 1,5,6,7-tetrahydro-4H-pyrrolo[3,2-c]pyridin-4-one trifluoroacetate | 0.0133 | |
| 42 | 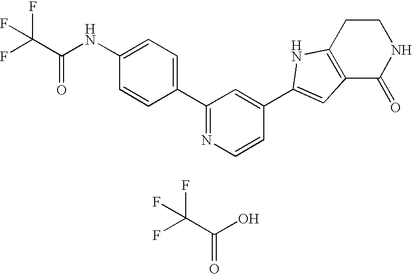 | 2,2,2-trifluoro-N-{4-[4-(4-oxo-4,5,6,7-tetrahydro-1H-pyrrolo[3,2- c]pyridin-2-yl)pyridin-2-yl]phenyl)acetamide trifluoroacetate | 0.0135 | |
| 43 | 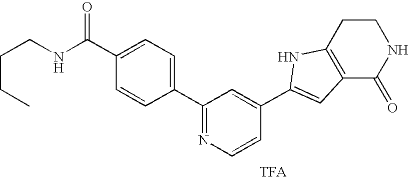 | N-butyl-4-[4-(4-oxo-4,5,6,7-tetrahydro-1H-pyrrolo[3,2-c]pyridin- 2-yl)pyridin-2-yl]benzamide trifluoroacetate | 0.0136 | |
| 44 | 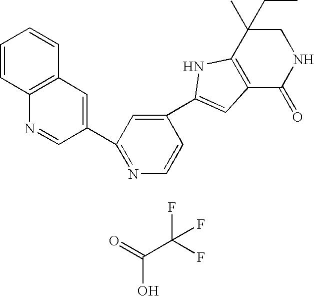 | 7-ethyl-7-methyl-2-(2-quinolin-3-ylpyridin-4-yl)-1,5,6,7- tetrahydro-4H-pyrrolo[3,2-c]pyridin-4-one trifluoroacetate | 0.0137 | |
| 45 | 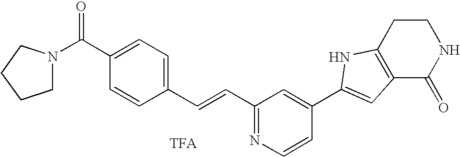 | 2-(2-{(E)-2-[4-(pyrrolidin-1-ylcarbonyl)phenyl]vinyl}pyridin-4-yl)- 1,5,6,7-tetrahydro-4H-pyrrolo[3,2-c]pyridin-4-one trifluoroacetate | 0.014 | |
| 46 | 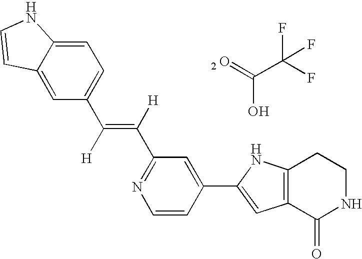 | 2-{2-[(E)-2-(1H-indol-5-yl)ethenyl]pyridin-4-yl}-1,5,6,7-tetrahydro 4H-pyrrolo[3,2-c]pyridin-4-one trifluoroacetate | 0.0141 | |
| 47 | 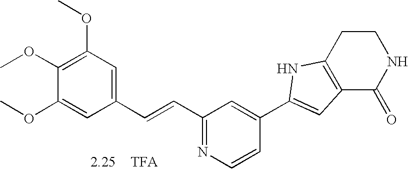 | 2-{2-[(E)-2-(3,4,5-trimethoxyphenyl)vinyl]pyridin-4-yl}-1,5,6,7- tetrahydro-4H-pyrrolo[3,2-c]pyridin-4-one trifluoroacetate | 0.0142 | |
| 48 | 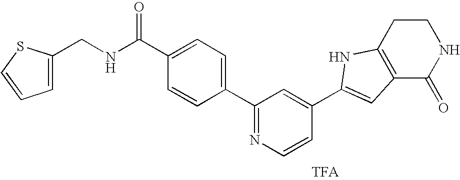 | 4-[4-(4-oxo-4,5,6,7-tetrahydro-1H-pyrrolo[3,2-c]pyridin-2- yl)pyridin-2-yl]-N-(thien-2-ylmethyl)benzamide trifluoroacetate | 0.0144 | |
| 49 | 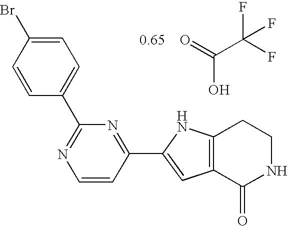 | 2-[2-(4-bromophenyl)pyrimidin-4-yl]-1,5,6,7-tetrahydro-4H- pyrrolo[3,2-c]pyridin-4-one trifluoroacetate | 0.0144 | |
| 50 | 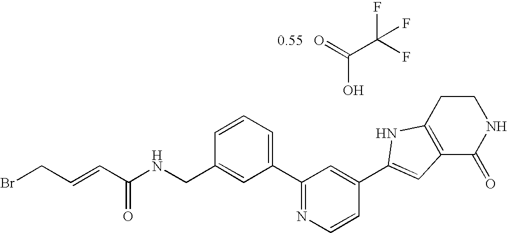 | (2E)-4-bromo-N-{3-[4-(4-oxo-4,5,6,7-tetrahydro-1H-pyrrolo[3,2- c]pyridin-2-yl)pyridin-2-yl]benzyl}but-2-enamide trifluoroacetate | 0.0146 | |
| 51 | 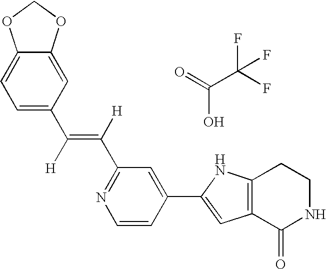 | 2-{2-[(E)-2-(1,3-benzodioxol-5-yl)ethenyl]pyridin-4-yl}-1,5,6,7- tetrahydro-4H-pyrrolo[3,2-c]pyridin-4-one trifluoroacetate | 0.0146 | |
| 52 | 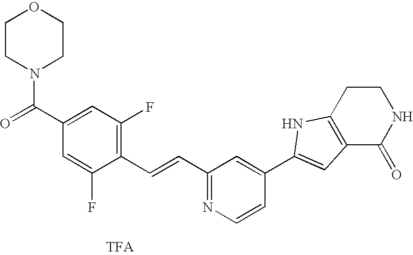 | 2-(2-{(E)-2-[2,6-difluoro-4-(morpholin-4- ylcarbonyl)phenyl]vinyl}pyridin-4-yl)-1,5,6,7-tetrahydro-4H- pyrrolo[3,2-c]pyridin-4-one trifluoroacetate | 0.0148 | |
| 53 | 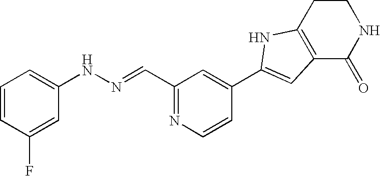 | 4-(4-oxo-4,5,6,7-tetrahydro-1H-pyrrolo[3,2-c]pyridin-2- yl)pyridine-2-carbaldehyde (3-fluorophenyl)hydrazone | 0.0153 | |
| 54 | 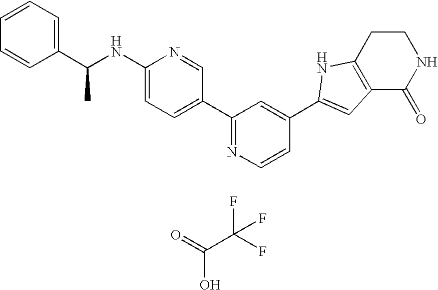 | 2-(6′-{[(1S)-1-phenylethyl]amino}-2,3′-bipyridin-4-yl)-1,5,6,7- tetrahydro-4H-pyrrolo[3,2-c]pyridin-4-one trifluoroacetate | 0.0154 | |
| 55 | 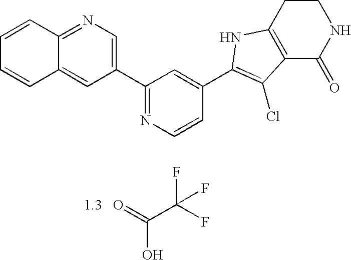 | 3-chloro-2-(2-quinolin-3-ylpyridin-4-yl)-1,5,6,7-tetrahydro-4H- pyrrolo[3,2-c]pyridin-4-one trifluoroacetate | 0.0156 | |
| 56 | 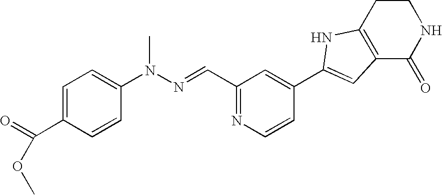 | methyl 4-((2E)-1-methyl-2-{[4-(4-oxo-4,5,6,7-tetrahydro-1H- pyrrolo[3,2-c]pyridin-2-yl)pyridin-2- yl]methylenelhydrazino)benzoate | 0.0156 | |
| 57 | 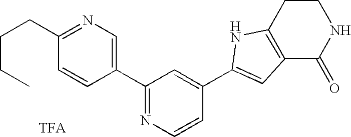 | 2-(6′-butyl-2,3′-bipyridin-4-yl)-1,5,6,7-tetrahydro-4H-pyrrolo[3,2- c]pyridin-4-one trifluoroacetate | 0.0157 | |
| 58 | 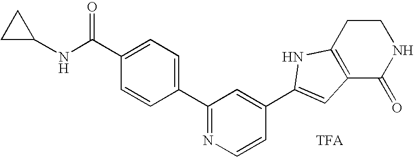 | N-cyclopropyl-4-[4-(4-oxo-4,5,6,7-tetrahydro-1H-pyrrolo[3,2- c]pyridin-2-yl)pyridin-2-yl]benzamide | 0.0158 | |
| 59 | 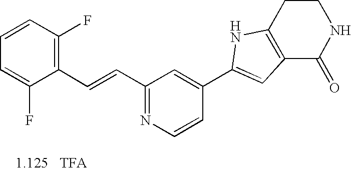 | 2-{2-[(E)-2-(2,6-difluorophenyl)vinyl]pyridin-4-yl}-1,5,6,7- tetrahydro-4H-pyrrolo[3,2-c]pyridin-4-one trifluoroacetate | 0.016 | |
| 60 | 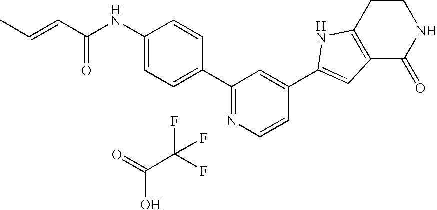 | (2E)-N-{4-[4-(4-oxo-4,5,6,7-tetrahydro-1H-pyrrolo[3,2-c]pyridin- 2-yl)pyridin-2-yl]phenyl}but-2-enamide trifluoroacetate | 0.0162 | |
| 61 | 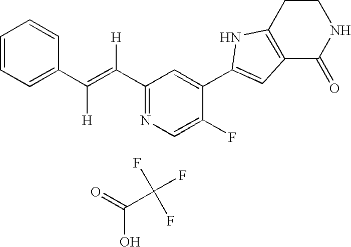 | 2-{5-fluoro-2-[(E)-2-phenylvinyl]pyridin-4-yl}-1,5,6,7-tetrahydro- 4H-pyrrolo[3,2-c]pyridin-4-one trifluoroacetate | 0.0162 | |
| 62 | 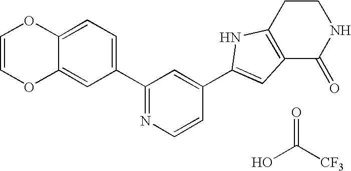 | 2-[2-(1,4-benzodioxin-6-yl)pyridin-4-yl]-1,5,6,7-tetrahydro-4H- pyrrolo[3,2-c]pyridin-4-one trifluoroacetate | 0.0164 | |
| 63 | 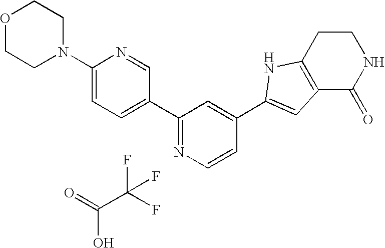 | 2-(6′-morpholin-4-yl-2,3-bipyridin-4-yl)-1,5,6,7-tetrahydro-4H- pyrrolo[3,2-c]pyridin-4-one trifluoroacetate | 0.0164 | |
| 64 | 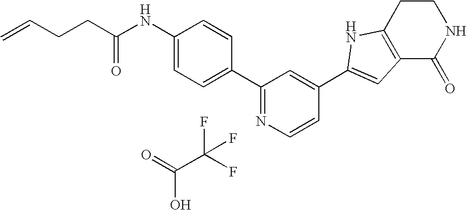 | N-{4-[4-(4-oxo-4,5,6,7-tetrahydro-1H-pyrrolo[3,2-c]pyridin-2- yl)pyridin-2-yl]phenyl}pent-4-enamide trifluoroacetate | 0.0167 | |
| 65 | 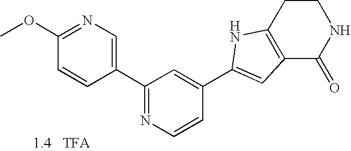 | 2-(6′-methoxy-2,3-bipyridin-4-yl)-1,5,6,7-tetrahydro-4H- pyrrolo[3,2-c]pyridin-4-one trifluoroacetate | 0.0168 | |
| 66 | 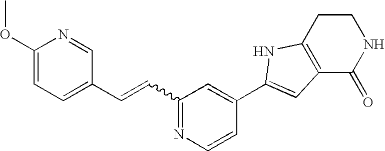 | 2-{2-[(E)-2-(6-methoxypyridin-3-yl)vinyl]pyridin-4-yl}-1,5,6,7- tetrahydro-4H-pyrrolo[3,2-c]pyridin-4-one | 0.017 | |
| 67 | 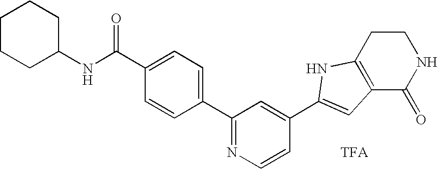 | N-cyclohexyl-4-[4-(4-oxo-4,5,6,7-tetrahydro-1H-pyrrolo[3,2- c]pyridin-2-yl)pyridin-2-yl]benzamide trifluoroacetate | 0.0171 | |
| 68 | 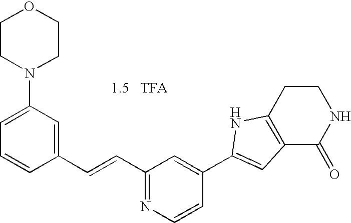 | 2-{2-[(E)-2-(3-morpholin-4-ylphenyl)vinyl]pyridin-4-yl}-1,5,6,7- tetrahydro-4H-pyrrolo[3,2-c]pyridin-4-one trifluoroacetate | 0.0171 | |
| 69 | 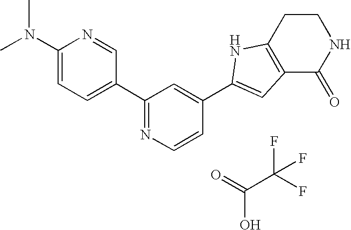 | 2-[6′-(dimethylamino)-2,3′-bipyridin-4-yl]-1,5,6,7-tetrahydro-4H- pyrrolo[3,2-c]pyridin-4-one trifluoroacetate | 0.0174 | |
| 70 | 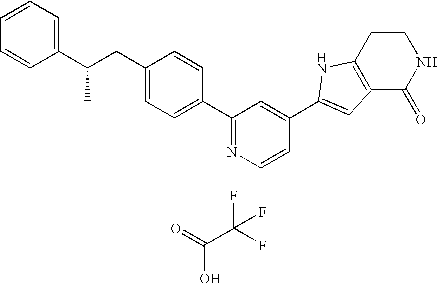 | 2-(6′-{[(1R)-1-phenylethyl]amino}-2,3′-bipyridin-4-yl)-1,5,6,7- tetrahydro-4H-pyrrolo[3,2-c]pyridin-4-one trifluoroacetate | 0.0175 | |
| 71 | 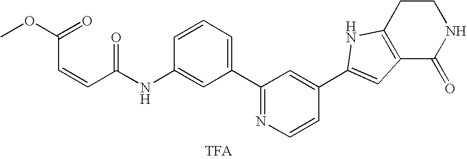 | methyl (2Z)-4-oxo-4-({3-[4-(4-oxo-4,5,6,7-tetrahydro-1H- pyrrolo[3,2-c]pyridin-2-yl)pyridin-2-yl]phenyl)amino)but-2-enoate trifluoroacetate | 0.0175 | |
| 72 | 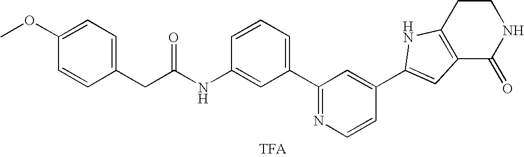 | 2-(4-methoxyphenyl)-N-{3-[4-(4-oxo-4,5,6,7-tetrahydro-1H- pyrrolo[3,2-c]pyridin-2-yl)pyridin-2-yl]phenyl}acetamide trifluoroacetate | 0.0176 | |
| 73 | 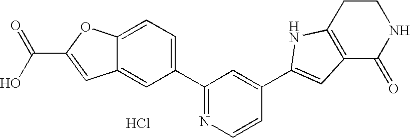 | 5-[4-(4-oxo-4,5,6,7-tetrahydro-1H-pyrrolo[3,2-c]pyridin-2- yl)pyridin-2-yl]-1-benzofuran-2-carboxylic acid hydrochloride | 0.0177 | |
| 74 | 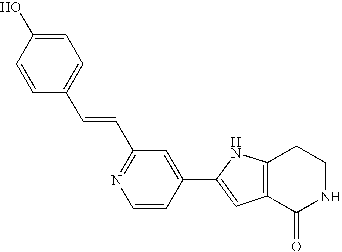 | 2-(2-[(E)-2-(4-hydroxyphenyl)ethenyl]pyridin-4-yl}-1,5,6,7- tetrahydro-4H-pyrrolo[3,2-c]pyridin-4-one | 0.0178 | |
| 75 | 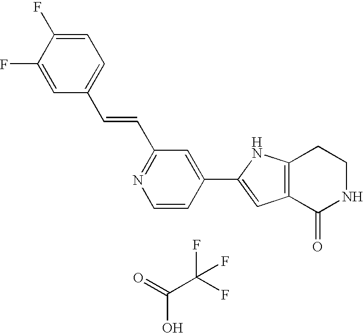 | 2-[2-[(E)-2-(3,4-difluorophenyl)ethenyl]pyridin-4-yl)-1,5,6,7- tetrahydro-4H-pyrrolo[3,2-c]pyridin-4-one trifluoroacetate | 0.018 | |
| 76 | 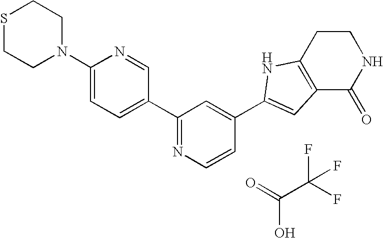 | 2-(6′-thiomorpholin-4-yl-2,3′-bipyridin-4-yl)-1,5,6,7-tetrahydro- 4H-pyrrolo[3,2-c]pyridin-4-one trifluoroacetate | 0.0181 | |
| 77 | 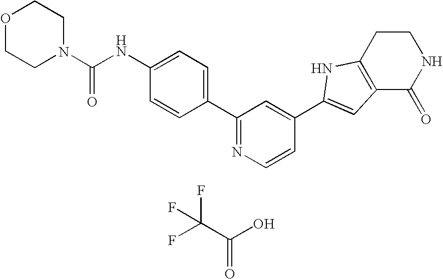 | N-{4-[4-(4-oxo-4,5,6,7-tetrahydro-1H-pyrrolo[3,2-c]pyridin-2- yl)pyridin-2-yl]phenyl}morpholine-4-carboxamide trifluoroacetate | 0.0181 | |
| 78 | 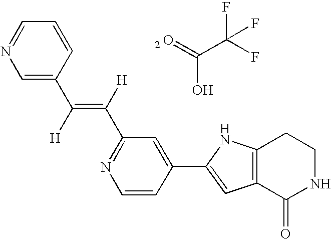 | 2-{2-[(E)-2-pyridin-3-ylvinyl]pyridin-4-yl}-1,5,6,7-tetrahydro-4H- pyrrolo[3,2-c]pyridin-4-one trifluoroacetate | 0.0183 | |
| 79 | 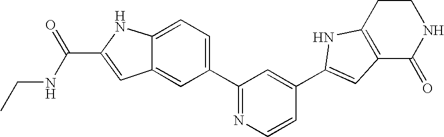 | N-ethyl-5-[4-(4-oxo-4,5,6,7-tetrahydro-1H-pyrrolo[3,2-c]pyridin- 2-yl)pyridin-2-yl]-1H-indole-2-carboxamide | 0.0184 | |
| 80 | 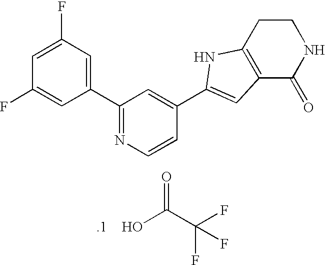 | 2-[2-(3,5-difluorophenyl)pyridin-4-yl]-1,5,6,7-tetrahydro-4H- pyrrolo[3,2-c]pyridin-4-one trifluoroacetate | 0.0185 | |
| 81 | 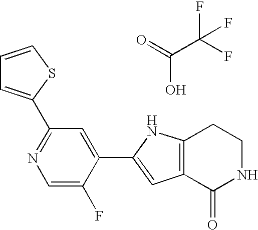 | 2-(5-fluoro-2-thien-2-ylpyridin-4-yl)-1,5,6,7-tetrahydro-4H- pyrrolo[3,2-c]pyridin-4-one trifluoroacetate | 0.0186 | |
| 82 | 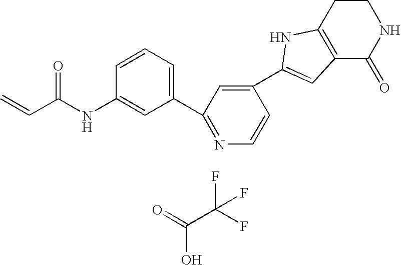 | N-{3-[4-(4-oxo-4,5,6,7-tetrahydro-1H-pyrrolo[3,2-c]pyridin-2- yl)pyridin-2-yl]phenyl}acrylamide trifluoroacetate | 0.0191 | |
| 83 | 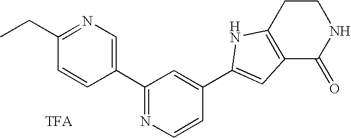 | 2-(6-ethyl-2,3′-bipyridin-4-yl)-1,5,6,7-tetrahydro-4H-pyrrolo[3,2- c]pyridin-4-one trifluoroacetate | 0.0191 | |
| 84 | 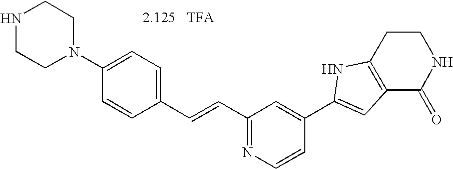 | 2-{2-[(E)-2-(4-piperazin-1-ylphenyl)vinyl]pyridin-4-yl}-1,5,6,7- tetrahydro-4H-pyrrolo[3,2-c]pyridin-4-one trifluoroacetate | 0.0191 | |
| 85 | 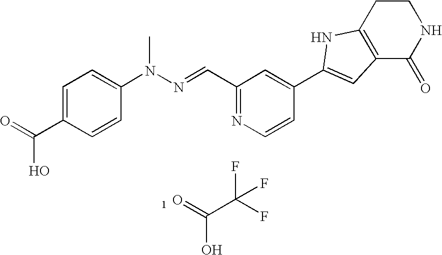 | 4-((2E)-1-methyl-2-{[4-(4-oxo-4,5,6,7-tetrahydro-1H-pyrrolo[3,2- c]pyridin-2-yl)pyridin-2-yl]methylene)hydrazino)benzoic acid trifluoroacetate | 0.0193 | |
| 86 | 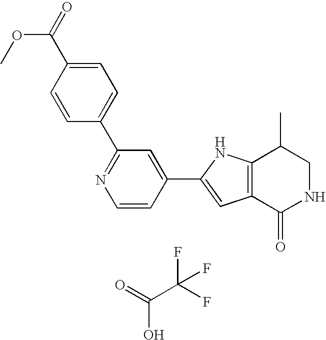 | methyl 4-[4-(7-methyl-4-oxo-4,5,6,7-tetrahydro-1H-pyrrolo[3,2- c]pyridin-2-yl)pyridin-2-yl]benzoate trifluoroacetate | 0.0195 | |
| 87 | 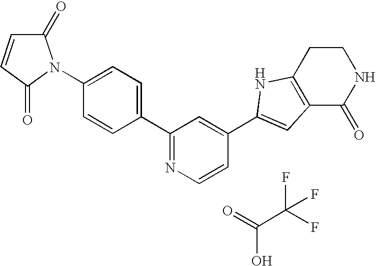 | 1-{4-[4-(4-oxo-4,5,6,7-tetrahydro-1H-pyrrolo[3,2-c]pyridin-2- yl)pyridin-2-yl]phenyl}-1H-pyrrole-2,5-dione trifluoroacetate | 0.0195 | |
| 88 | 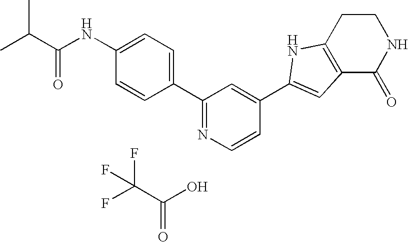 | 2-methyl-N-{4-[4-(4-oxo-4,5,6,7-tetrahydro-1H-pyrrolo[3,2- c]pyridin-2-yl)pyridin-2-yl]phenyl}propanamide trifluoroacetate | 0.0195 | |
| 89 | 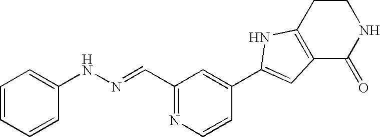 | 4-(4-oxo-4,5,6,7-tetrahydro-1H-pyrrolo[3,2-c]pyridin-2- yl)pyridine-2-carbaldehyde phenylhydrazone | 0.0197 | |
| 90 | 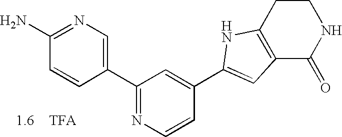 | 2-(6′-amino-2,3′-bipyridin-4-yl)-1,5,6,7-tetrahydro-4H-pyrrolo[3,2- c]pyridin-4-one trifluoroacetate | 0.0199 | |
| 91 | 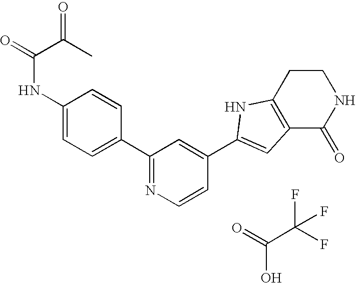 | 2-methyl-N-{4-[4-(4-oxo-4,5,6,7-tetrahydro-1H-pyrrolo[3,2- c]pyridin-2-yl)pyridin-2-yl]phenyl}acrylamide trifluoroacetate | 0.0201 | |
| 92 | 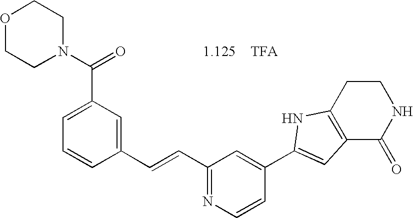 | 2-(2-{(E)-2-[3-(morpholin-4-ylcarbonyl)phenyl]vinyl}pyridin-4-yl)- 1,5,6,7-tetrahydro-4H-pyrrolo[3,2-c]pyridin-4-one trifluoroacetate | 0.0201 | |
| 93 | 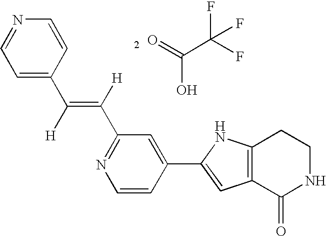 | 2-{2-[(E)-2-pyridin-4-ylethenyl]pyridin-4-yl}-1,5,6,7-tetrahydro- 4H-pyrrolo[3,2-c]pyridin-4-one trifluoroacetate | 0.0202 | |
| 94 | 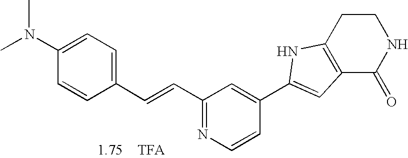 | 2-(2-{(E)-2-[4-(dimethylamino)phenyl]vinyl}pyridin-4-yl)-1,5,67- tetrahydro-4H-pyrrolo[3,2-c]pyridin-4-one trifluoroacetate | 0.0203 | |
| 95 | 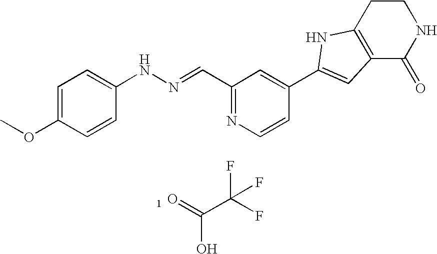 | 4-(4-oxo-4,5,6,7-tetrahydro-1H-pyrrolo[3,2-c]pyridin-2- yl)pyridine-2-carbaldehyde (4-methoxyphenyl)hydrazone trifluoroacetate | 0.0204 | |
| 96 | 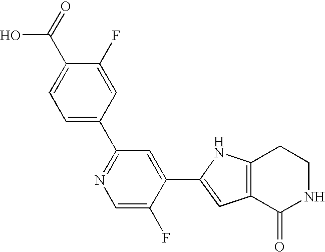 | 2-fluoro-4-[5-fluoro-4-(4-oxo-4,5,6,7-tetrahydro-1H-pyrrolo[3,2- c]pyridin-2-yl)pyridin-2-yl]benzoic acid | 0.0205 | |
| 97 | 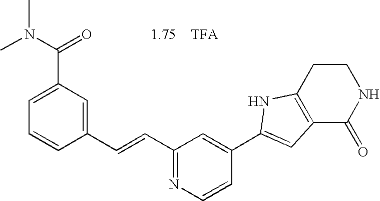 | N,N-dimethyl-3-{(E)-2-[4-(4-oxo-4,5,6,7-tetrahydro-1H- pyrrolo[3,2-c]pyridin-2-yl)pyridin-2-yl]vinyl}benzamide trifluoroacetate | 0.0206 | |
| 98 | 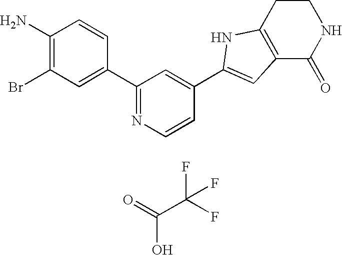 | 2-[2-(4-amino-3-bromophenyl)pyridin-4-yl]-1,5,6,7-tetrahydra- 4H-pyrrolo[3,2-c]pyridin-4-one trifluoroacetate | 0.0209 | |
| 99 | 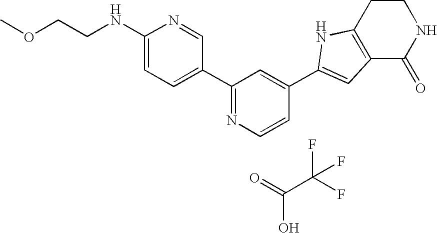 | 2-{6′-[(2-methoxyethyl)amino]-2,3′-bipyridin-4-yl}-1,5,6,7- tetrahydro-4H-pyrrolo[3,2-c]pyridin-4-one trifluoroacetate | 0.0211 | |
| 100 | 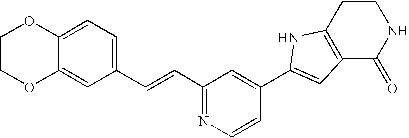 | 2-{2-[(E)-2-(2,3-dihydro-1,4-benzodioxin-6-yl)vinyl]pyridin-4-yl}- 1,5,6,7-tetrahydro-4H-pyrrolo[3,2-c]pyridin-4-one | 0.0211 | |
| 101 | 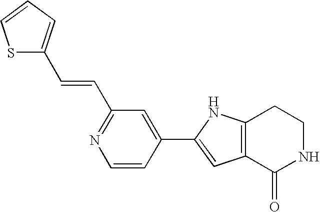 | 2-{2-[(E)-2-thien-2-ylethenyl]pyridin-4-yl}-1,5,6,7-tetrahydro-4H- pyrrolo[3,2-c]pyridin-4-one | 0.0212 | |
| 102 | 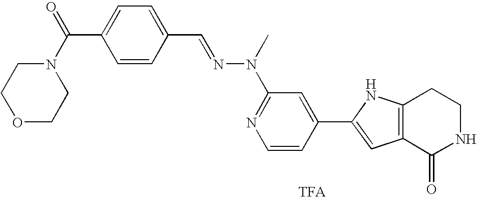 | 4-(morpholin-4-ylcarbonyl)benzaldehyde methyl[4-(4-oxo- 4,5,6,7-tetrahydro-1H-pyrrolo[3,2-c]pyridin-2-yl)pyridin-2- yl]hydrazone trifluoroacetate | 0.0215 | |
| 103 | 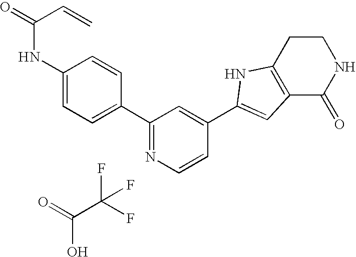 | N-{4-[4-(4-oxo-4,5,6,7-tetrahydro-1H-pyrrolo[3,2-c]pyridin-2- yl)pyridin-2-yl]phenyl}acrylamide trifluoroacetate | 0.0217 | |
| 104 | 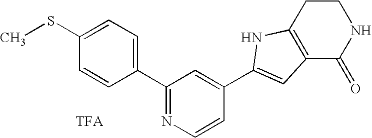 | 2-{2-[4-(methylthio)phenyl]pyridin-4-yl}-1,5,6,7-tetrahydro-4H- pyrrolo[3,2-c]pyridin-4-one trifluoroacetate | 0.022 | |
| 105 | 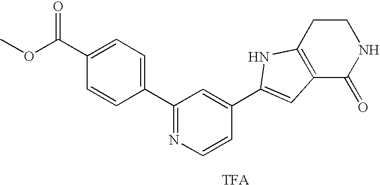 | methyl 4-[4-(4-oxo-4,5,6,7-tetrahydro-1H-pyrrolo[3,2-c]pyridin-2- yl)pyridin-2-yl]benzoate trifluoroacetate | 0.022 | |
| 106 | 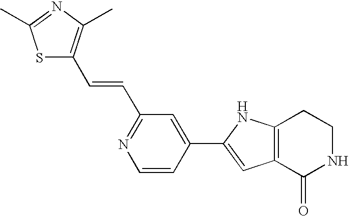 | 2-(2-[(E)-2-(2,4-dimethyl-1,3-thiazol-5-yl)vinyl]pyridin-4-yl}- 1,5,6,7-tetrahydro-4H-pyrrolo[3,2-c]pyridin-4-one | 0.0221 | |
| 107 | 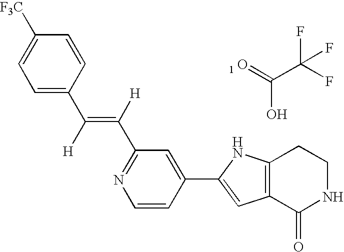 | 2-(2-{(E)-2-[4-(trifluoromethyl)phenyl]ethenyl}pyridin-4-yl)- 1,5,6,7-tetrahydro-4H-pyrrolo[3,2-c]pyridin-4-one | 0.0223 | |
| 108 | 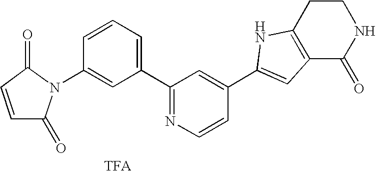 | 1-{3-[4-(4-oxo-4,5,6,7-tetrahydro-1H-pyrrolo[3,2-c]pyridin-2- yl)pyridin-2-yl]phenyl}-1H-pyrrole-2,5-dione trifluoroacetate | 0.0224 | |
| 109 | 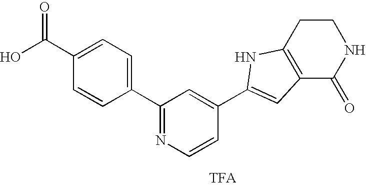 | 4-[4-(4-oxo-4,5,6,7-tetrahydro-1H-pyrrolo[3,2-c]pyridin-2- yl)pyridin-2-yl]benzaic acid trifluoroacetate | 0.0225 | |
| 110 | 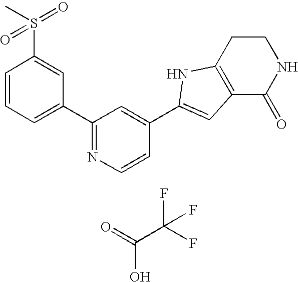 | 2-{2-[3-(methylsulfonyl)phenyl]pyridin-4-yl}-1,5,6,7-tetrahydro- 4H-pyrrolo(3,2-c]pyridin-4-one trifluoroacetate | 0.0225 | |
| 111 | 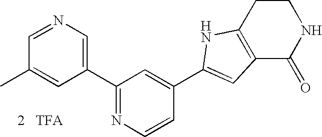 | 2-(5′-methyl-2,3′-bipyridin-4-yl)-1,5,6,7-tetrahydro-4H- pyrrolo[3,2-c]pyridin-4-one trifluoroacetate | 0.0225 | |
| 112 | 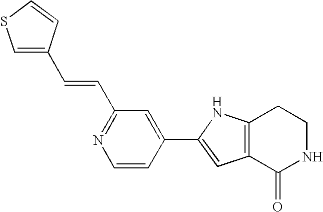 | 2-{2-[(E)-2-thien-3-ylvinyl]pyridin-4-yl}-1,5,6,7-tetrahydro-4H- pyrrolo(3,2-c]pyridin-4-one | 0.0227 | |
| 113 | 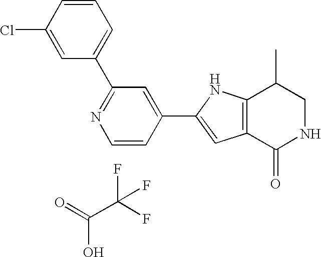 | 2-[2-(3-chlorophenyl)pyridin-4-yl]-7-methyl-1,5,6,7-tetrahydro- 4H-pyrrolo[3,2-c]pyridin-4-one trifluoroacetate | 0.0228 | |
| 114 | 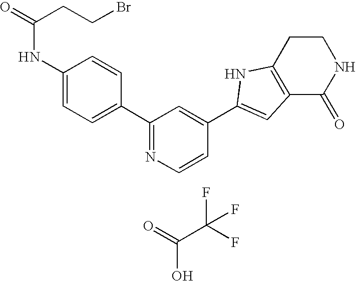 | 3-bromo-N-{4-[4-(4-oxo-4,5,6,7-tetrahydro-1H-pyrrolo[3,2- c]pyridin-2-yl)pyridin-2-yl]phenyl}propanamide trifluoroacetate | 0.0228 | |
| 115 | 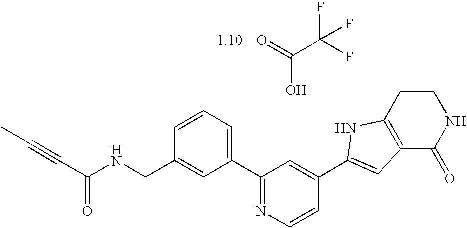 | N-{3-[4-(4-oxo-4,5,6,7-tetrahydro-1H-pyrrolo[3,2-c]pyridin-2- yl)pyridin-2-yl]benzyl}but-2-ynamide trifluoroacetate | 0.023 | |
| 116 | 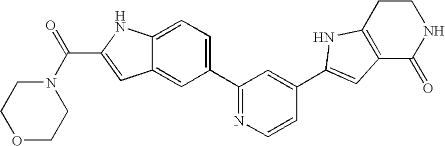 | 2-{2-[2-(morpholin-4-ylcarbonyl)-1H-indol-5-yl]pyridin-4-yl}- 1,5,6,7-tetrahydro-4H-pyrrolo[3,2-c]pyridin-4-one | 0.0234 | |
| 117 | 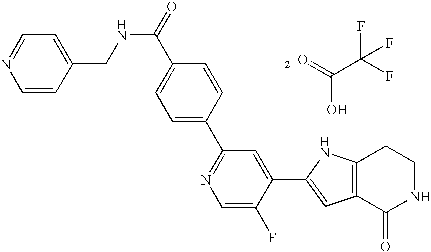 | 4-[5-fluoro-4-(4-oxo-4,5,6,7-tetrahydro-1H-pyrrolo[3,2-c]pyridin- 2-yl)pyridin-2-yl]-N-(pyridin-4-ylmethyl)benzamide trifluoroacetate | 0.0234 | |
| 118 | 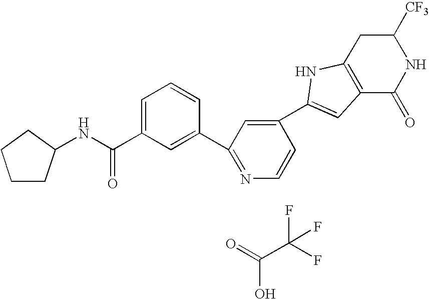 | N-cyclopentyl-3-{4-[4-oxo-6-(trifluoromethyl)-4,5,6,7-tetrahydro- 1H-pyrrolo[3,2-c]pyridin-2-yl]pyridin-2-yl)benzamide trifluoroacetate | 0.0236 | |
| 119 | 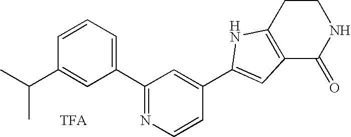 | 2-[2-(3-isopropylphenyl)pyridin-4-yl]-1,5,6,7-tetrahydro-4H- pyrrolo[3,2-c]pyridin-4-one trifluoroacetate | 0.0237 | |
| 120 | 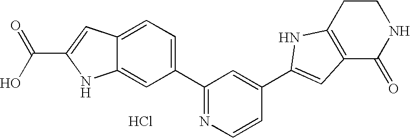 | 6-[4-(4-oxo-4,5,6,7-tetrahydro-1H-pyrrolo[3,2-c]pyridin-2- yl)pyridin-2-yl]-1H-indole-2-carboxylic acid hydrochloride | 0.0239 | |
| 121 | 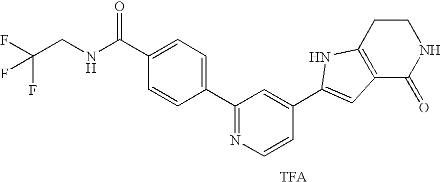 | 4-[4-(4-oxo-4,5,6,7-tetrahydro-1H-pyrrolo[3,2-c]pyridin-2- yl)pyridin-2-yl]-N-(2,2,2-trifluoroethyl)benzamide trifuoroacetate | 0.0239 | |
| 122 | 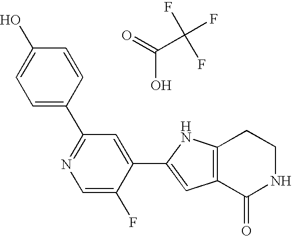 | 2-[5-fluoro-2-(4-hydroxyphenyl)pyridin-4-yl]-1,5,6,7-tetrahydro- 4H-pyrrolo[3,2-c]pyridin-4-one trifluoroacetate | 0.024 | |
| 123 | 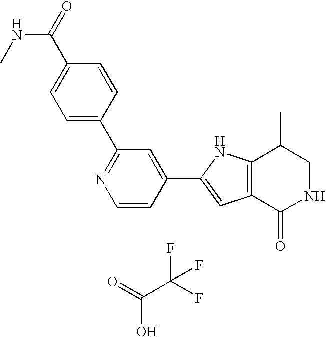 | N-methyl-4-[4-(7-methyl-4-oxo-4,5,6,7-tetrahydro-1H- pyrrolo[3,2-clpyridin-2-yl)pyridin-2-yl]benzamide trifluoroacetate | 0.0241 | |
| 124 | 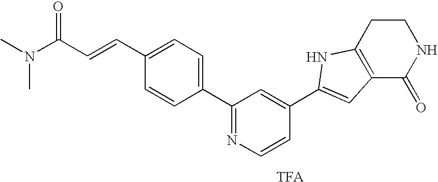 | (2E)-N,N-dimethyl-3-{4-[4-(4-oxo-4,s,6,7-tetrahydro-1H- pyrrolo[3,2-c]pyridin-2-y)pyridin-2-yl]phenyl}acrylamide trifluoroacetate | 0.0243 | |
| 125 | 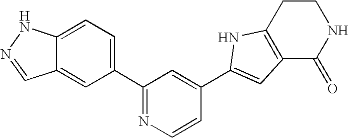 | 2-[2-(1H-indazol-5-yl)pyridin-4-yl]-1,5,6,7-tetrahydro-4H- pyrrolo[3,2-c]pyridin-4-one | 0.0244 | |
| 126 | 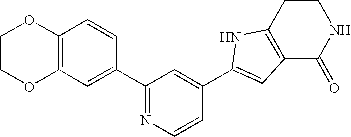 | 2-[2-(2,3-dihydro-1,4-benzodioxin-6-yl)pyridin-4-yl]-1,5,6,7- tetrahydro-4H-pyrrolo(3,2-c]pyridin-4-one | 0.0248 | |
| 127 | 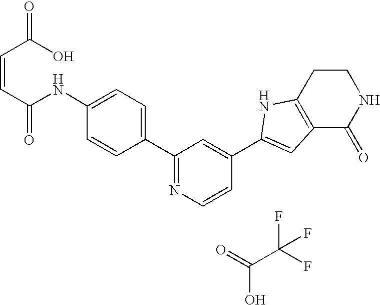 | (2Z)-4-oxo-4-({4-[4-(4-oxo-4,5,6,7-tetrahydro-1H-pyrrolo[3,2- c]pyridin-2-yl)pyridin-2-yl]phenyl}amino)but-2-enoic acid trifluoroacetate | 0.0248 | |
| 128 | 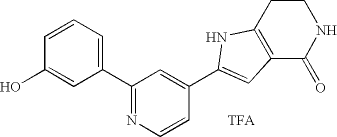 | 2-[2-(3-hydroxyphenyl)pyridin-4-yl]-1,5,6,7-tetrahydro-4H- pyrrolo[3,2-c]pyridin-4-one trifluoroacetate | 0.0249 | |
| 129 | 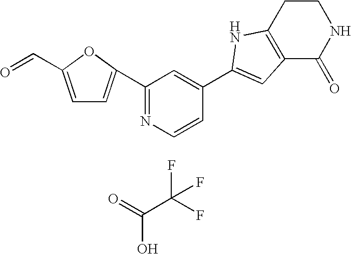 | 5-[4-(4-oxo-4,5,6,7-tetrahydro-1H-pyrrolo[3,2-]pyridin-2- yl)pyridin-2-yl]-2-furaldehyde trifluoroacetate | 0.0252 | |
| 130 | 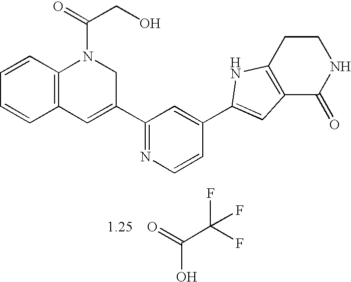 | 2-[2-(1-glycoloyl-1,2-dihydroquinolin-3-yl)pyridin-4-yl]-1,5,6,7- tetrahydro-4H-pyrrolo[3,2-c]pyridin-4-one trifluoroacetate | 0.0252 | |
| 131 | 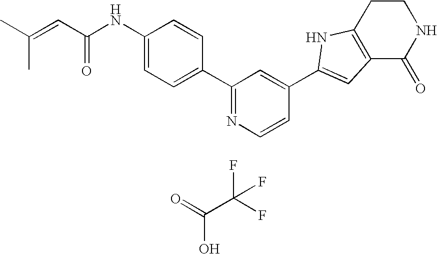 | 3-methyl-N-{4-[4-(4-oxo-4,5,6,7-tetrahydro-1H-pyrrolo[3,2- c]pyridin-2-yl)pyridin-2-yl]phenyl}but-2-enamide trifluoroacetate | 0.0256 | |
| 132 | 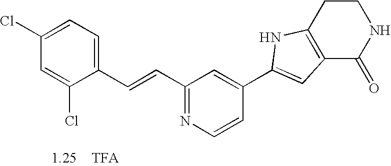 | 2-{2-[(E)-2-(2,4-dichlorophenyl)vinyl]pyridin-4-yl}-1,5,6,7- tetrahydro-4H-pyrrolo[3,2-c]pyridin-4-one trifluoroacetate | 0.0256 | |
| 133 | 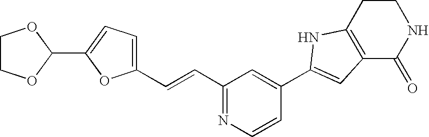 | 2-(2-{(E)-2-[5-(1,3-dioxolan-2-yl)-2-furyl]vinyl}pyridin-4-yl)- 1,5,6,7-tetrahydro-4H-pyrrolo[3,2-c]pyridin-4-one | 0.0256 | |
| 134 | 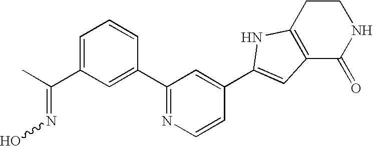 | 2-(2-{3-[(1E)-N-hydroxyethanimidoyl]phenyl}pyridin-4-yl)-1,5,6,7- tetrahydro-4H-pyrrolo[3,2-c]pyridin-4-one | 0.0257 | |
| 135 | 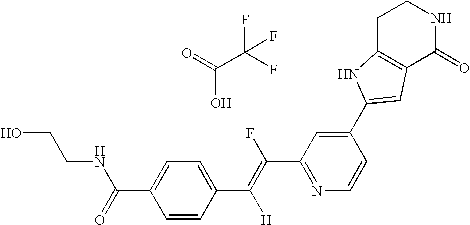 | 4-{(Z)-2-fluoro-2-[4-(4-oxo-4,5,6,7-tetrahydro-1H-pyrrolo[3,2- c]pyridin-2-yl)pyridin-2-yl]vinyl}-N-(2-hydroxyethyl)benzamide trifluoroacetate or 4-{(Z)-2-fluoro-2-[4-(4-oxo-4,5,6,7-tetrahydro- 1H-pyrrolo[3,2-c]pyridin-2-yl)pyridin-2-yl]vinyl}-N-(2- hydroxyethyl)benzamide trifluoroacetate | 0.0257 | |
| 136 | 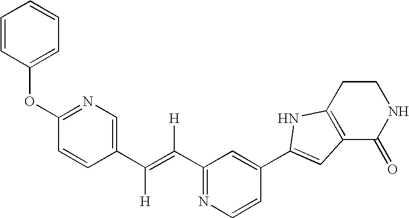 | 2-{2-[(E)-2-(6-phenoxypyridin-3-yl)vinyl]pyridin-4-yl}-1,5,6,7- tetrahydra-4H-pyrrolo[3,2-c]pyridin-4-one | 0.0262 | |
| 137 | 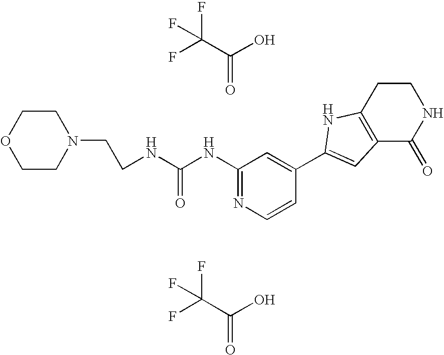 | N-(2-morpholin-4-ylethyl)-N′-{4-[4-(4-oxo-4,5,6,7-tetrahydro-1H- pyrroIo[3,2-c]pyridin-2-yl)pyridin-2-yl]phenyl}urea bis(trifluoroacetate) | 0.0264 | |
| 138 | 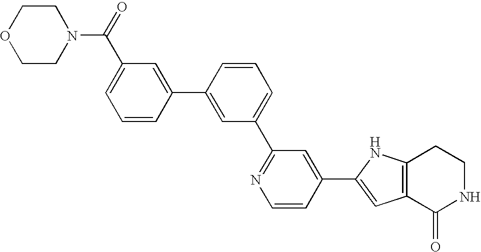 | 2-{2-[3′-(morpholin-4-ycarbonyl)-1,1′-biphenyl-3-yl]pyridin-4-yl}1,5,6,7-tetrahydro-4H-pyrrolo[3,2-c]pyridin-4-one | 0.0265 | |
| 139 | 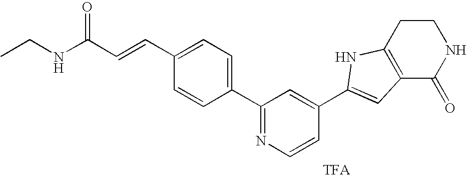 | (2E)-N-ethyl-3-{4-[4-(4-oxo-4,5,6,7-tetrahydro-1H-pyrrolo[3,2- c]pyridin-2-yl)pyridin-2-yl]phenyl}acrylamide trifluoroacetate | 0.0265 | |
| 140 | 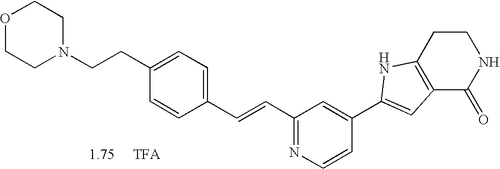 | 2-(2-{(E)-2-[4-(2-morpholin-4-ylethyl)phenyl]vinyl}pyridin-4-yl)- 1,5,6,7-tetrahydro-4H-pyrrolo(3,2-c]pyridin-4-one trifluoroacetate | 0.0265 | |
| 141 | 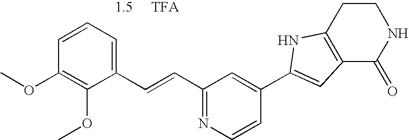 | 2-{2-[(E)-2-(2,3-dimethoxyphenyl)vinyl]pyridin-4-yl}-1,5,6,7- tetrahydro-4H-pyrrolo[3,2-c]pyridin-4-one trifluoroacetate | 0.0266 | |
| 142 | 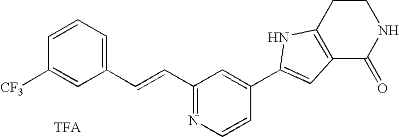 | 2-(2-{(E)-2-[3-(trifluoromethyl)phenyl]vinyl)pyridin-4-yl)-1,5,6,7- tetrahydro-4H-pyrrolo[3,2-c]pyridin-4-one trifluoroacetate | 0.0266 | |
| 143 | 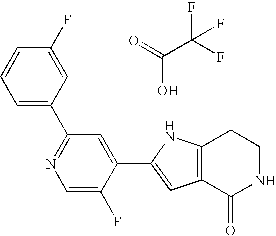 | 2-[5-fluoro-2-(3-fluorophenyl)pyridin-4-yl]-1,5,6,7-tetrahydro-4H- pyrrolo[3,2-c]pyridin-4-one trifluoroacetate | 0.0267 | |
| 144 | 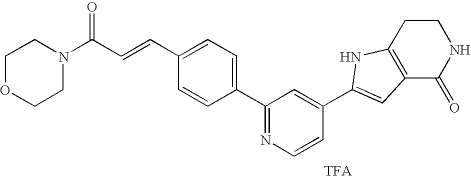 | 2-(2-{4-[(1E)-3-morpholin-4-yl-3-oxoprop-1-enyl]phenyl}pyridin- 4-yl)-1,5,6,7-tetrahydro-4H-pyrrolo[3,2-c]pyridin-4-one trifluoroacetate | 0.0269 | |
| 145 | 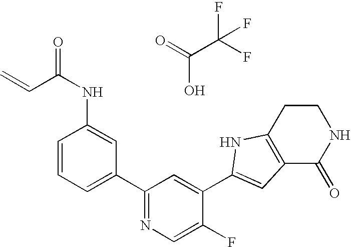 | N-{3-[5-fluoro-4-(4-axo-4,5,6,7-tetrahydro-1H-pyrrolo[3,2- c]pyridin-2-yl)pyridin-2-yl]phenyl}acrylamide trifluoroacetate | 0.0269 | |
| 146 | 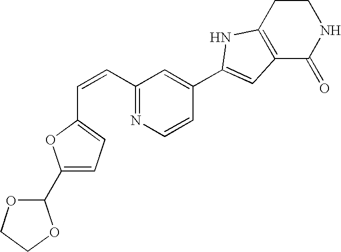 | 2-(2-{(Z)-2-[5-(1,3-dioxolan-2-yl)-2-furyl]vinyl}pyridin-4-yl)-1,5,6,7-tetrahydro-4H-pyrrolo[3,2-c]pyridin-4-one | 0.0269 | |
| 147 | 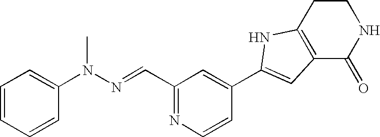 | 4-(4-oxo-4,5,6,7-tetrahydro-1H-pyrrolo[3,2-c]pyridin-2- yl)pyridine-2-carbaldehyde methyl(phenyl)hydrazone | 0.0271 | |
| 148 | 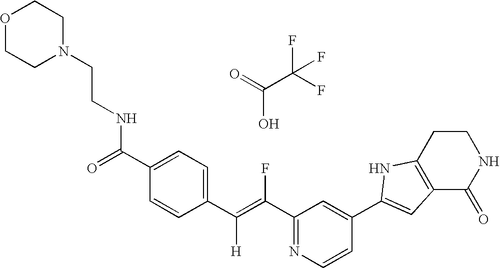 | 4-{(Z)-2-fluoro-2-(4-(4-oxo-4,5,6,7-tetrahydro-1H-pyrrolo[3,2- c]pyridin-2-yl)pyridin-2-yl]vinyl}-N-(2-morpholin-4- ylethyl)benzamide trifluoroacetate | 0.0274 | |
| 149 | 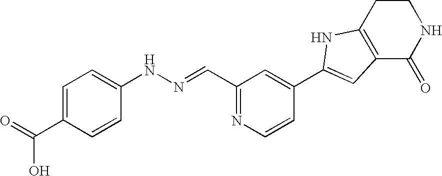 | 4-((2E)-2-{[4-(4-oxo-4,5,6,7-tetrahydro-1H-pyrrolo[3,2-c]pyridin- 2-yl)pyridin-2-yl]methylene}hydrazino)benzoic acid | 0.0274 | |
| 150 | 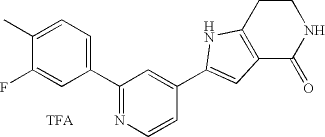 | 2-[2-(3-fluoro-4-methylphenyl)pyrdin-4-yl]-1,5,6,7-tetrahydro-4H- pyrrolo[3,2-c]pyridin-4-one trifluoroacetate | 0.0276 | |
| 151 | 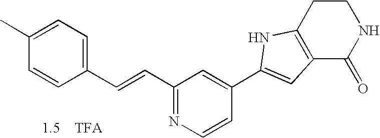 | 2-{2-[(E)-2-(4-methylphenyl)vinyl]pyridin-4-yl)-1,5,6,7-tetrahydro- 4H-pyrrolo[3,2-c]pyridin-4-one trifluoroacetate | 0.0276 | |
| 152 | 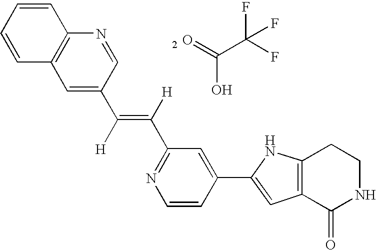 | 2-{2-[(E)-2-quinolin-3-ylethenyl]pyridin-4-yl}-1,5,6,7-tetrahydro- 4H-pyrrolo[3,2-c]pyridin-4-one trifluoroacetate | 0.0278 | |
| 153 | 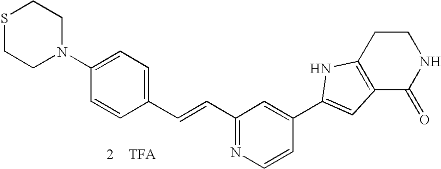 | 2-{2-[(E)-2-(4-thiomorpholin-4-ylphenyl)yinyl]pyridin-4-yl)- 1,5,6,7-tetrahydro-4H-pyrrolo[3,2-c]pyridin-4-one trifluoroacetate | 0.0278 | |
| 154 | 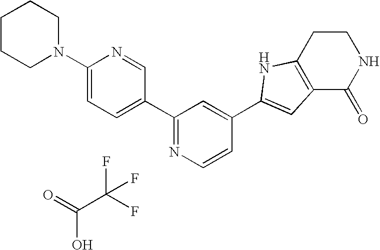 | 2-(6′-pipendin-1-yl-2,3′-bipyridin-4-yl)-1,5,6,7-tetrahydro-4H- pyrrolo[3,2-c]pyridin-4-one trifluoroacetate | 0.0279 | |
| 155 | 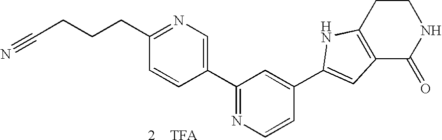 | 4-[4-(4-oxo-4,5,6,7-tetrahydro-1H-pyrrolo[3,2-c]pyridin-2-yl)-2,3′bipyridin-6′-yl]butanenitrile trifluoroacetate | 0.028 | |
| 156 | 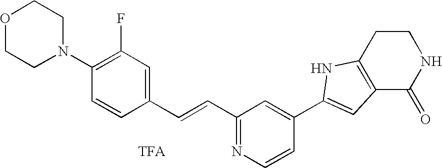 | 2-{2-[(E)-2-(3-fluoro-4-morpholin-4-ylphenyl)vinyl]pyridin-4-yl}- 1,5,6,7-tetrahydro-4H-pyrrolo[3,2-c]pyridin-4-one trifluoroacetate | 0.0281 | |
| 157 | 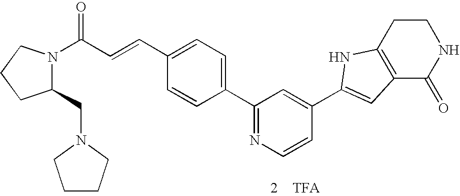 | 2-[2-(4-{(1E)-3-oxo-3-[(2R)-2-(pyrrolidin-1-ylmethyl)pyrrolidin-1- yl]prop-1-enyl}phenyl)pyridin-4-yl]-1,5,6,7-tetrahydro-4H- pyrrolo[3,2-c]pyridin-4-one trifluoroacetate | 0.0282 | |
| 158 | 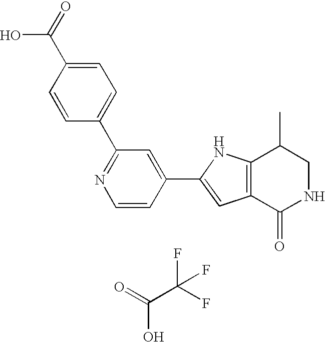 | 4-[4-(7-methyl-4-oxo-4,5,6,7-tetrahydro-1H-pyrrolo[3,2-c]pyridin- 2-yl)pyridin-2-yl]benzoic acid trifluoroacetate | 0.0283 | |
| 159 | 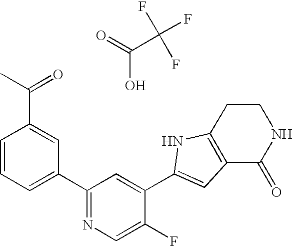 | 2-[2-(3-acetylphenyl)-5-fluoropyridin-4-yl]-1,5,6,7-tetrahydro-4H- pyrrolo[3,2-c]pyridin-4-one trifluoroacetate | 0.0283 | |
| 160 | 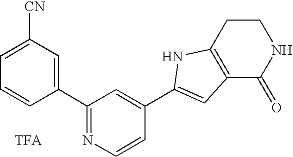 | 3-[4-(4-oxo-4,5,6,7-tetrahydro-1H-pyrrolo[3,2-c]pyridin-2- yl)pyridin-2-yl]benzonitrile trifluoroacetate | 0.0285 | |
| 161 | 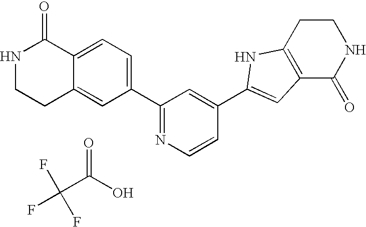 | 6-[4-(4-oxo-4,5,6,7-tetrahydro-1H-pyrrolo[3,2-c]pyridin-2- yl)pyridin-2-yl]-3,4-dihydroisoquinolin-1(2H)-one trifluoroacetate | 0.0286 | |
| 162 | 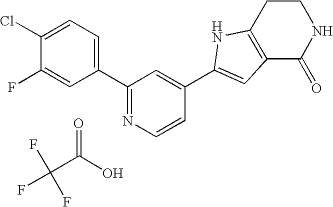 | 2-[2-(4-chloro-3-fluorophenyl)pyridin-4-yl]-1,5,6,7-tetrahydro-4H- pyrrolo[3,2-c]pyridin-4-one trifluoroacetate | 0.029 | |
| 163 | 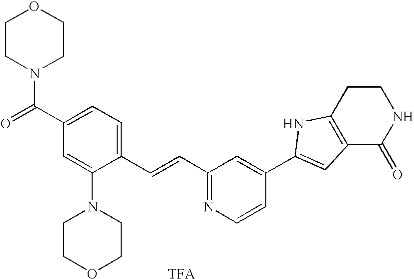 | 2-(2-{(E)-2-[2-morpholin-4-yl-4-(morpholin-4- ylcarbonyl)phenyl]vinyl)pyridin-4-yl)-1,5,6,7-tetrahydro-4H- pyrrolo[3,2-c]pyridin-4-one trifluoroacetate | 0.0291 | |
| 164 | 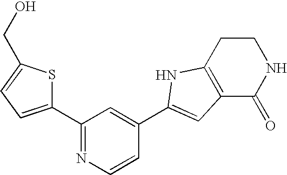 | 2-{2-[5-(hydroxymethyl)thien-2-yl]pyridin-4-yl}-1,5,6,7-tetrahydro- 4H-pyrrolo[3,2-c]pyridin-4-one | 0.0294 | |
| 165 | 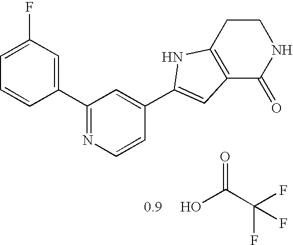 | 2-[2-(3-fluorophenyl)pyridin-4-yl]-1,5,6,7-tetrahydro-4H- pyrrolo[3,2-c]pyridin-4-one trifluoroacetate | 0.0294 | |
| 166 | 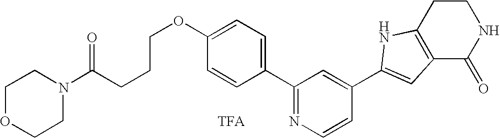 | 2-{2-[4-(4-morpholin-4-yl-4-oxobutoxy)phenyl]pyridin-4-yl}- 1,5,6,7-tetrahydro-4H-pyrrolo[3,2-c]pyridin-4-one trifluoroacetate | 0.0294 | |
| 167 | 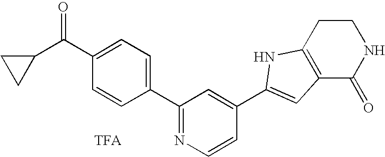 | 2-{2-[4-(cyclopropylcarbonyl)phenyl]pyridin-4-yl}-1,5,6,7- tetrahydro-4H-pyrrolo[3,2-c]pyridin-4-one trifluoroacetate | 0.0295 | |
| 168 | 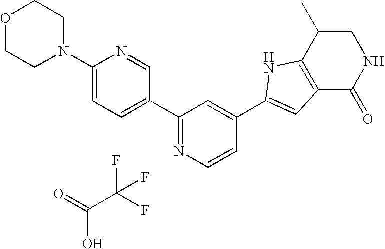 | 7-methyl-2-(6′-morpholin-4-yl-2,3′-bipyridin-4-yl)-1,5,6,7- tetrahydro-4H-pyrrolo[3,2-c]pyridin-4-one trifluoroacetate | 0.0296 | |
| 169 | 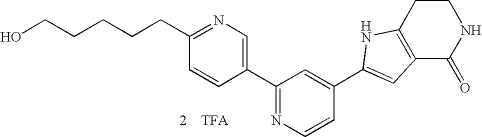 | 2-[6′-(5-hydroxypentyl)-2,3′-bipyridin-4-yl]-1,5,6,7-tetrahydro-4H- pyrrolo[3,2-c]pyridin-4-one trifluoroacetate | 0.0296 | |
| 170 | 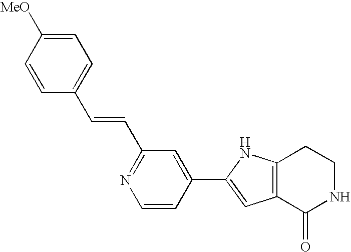 | 2-{2-[(E)-2-(4-methoxyphenyl)ethenyl]pyridin-4-yl}-1,5,6,7- | tetrahydro-4H-pyrrolo[3,2-c]pyridin-4-one | 0.0296 |
| 171 | 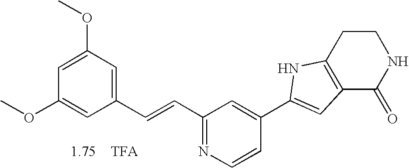 | 2-{2-[(E)-2-(3,5-dimethoxyphenyl)vinyl]pyridin-4-yl}-1,5,6,7- tetrahydro-4H-pyrrolo[3,2-c]pyridin-4-one trifluoroacetate | 0.0296 | |
| 172 | 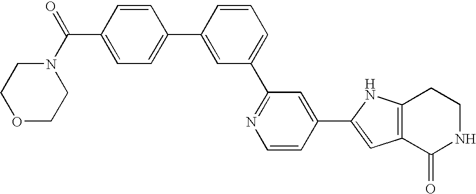 | 2-{2-[4′-(morpholin-4-ylcarbonyl)-1,1′-biphenyl-3-yl]pyridin-4-yl}- 1,5,6,7-tetrahydro-4H-pyrrolo[3,2-c]pyridin-4-one | 0.0297 | |
| 173 | 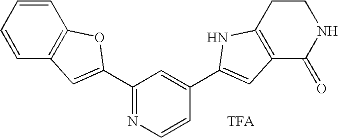 | 2-[2-(1-benzofuran-2-yl)pyridin-4-yl]-1,5,6,7-tetrahydro-4H- pyrrolo[3,2-c]pyridin-4-one trifluoroacetate | 0.0298 | |
| 174 | 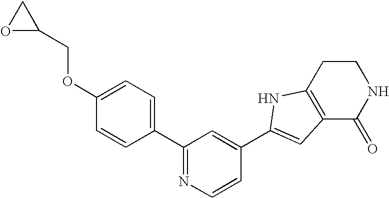 | 2-{2-[4-(oxiran-2-ylmethoxy)phenyl]pyridin-4-yl)-1,5,6,7- tetrahydro-4H-pyrrolo[3,2-c]pyridin-4-one | 0.0298 | |
| 175 | 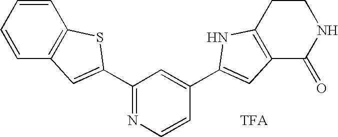 | 2-[2-(1-benzothien-2-yl)pyridin-4-yl]-1,5,6,7-tetrahydro-4H- pyrrolo[3,2-c]pyridin-4-one trifluoroacetate | 0.0302 | |
| 176 | 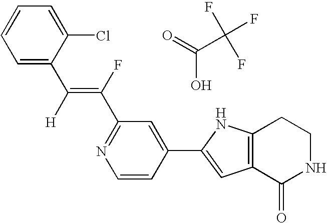 | 2-{2-[(Z)-2-(2-chlorophenyl)-1-fluorovinyl]pyridin-4-yl}-1,5,6,7- tetrahydro-4H-pyrrolo[3,2-c]pyridin-4-one trifluoroacetate | 0.0303 | |
| 177 | 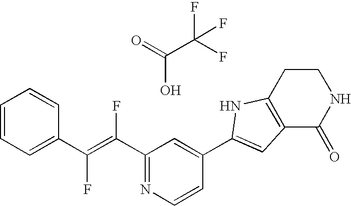 | 2-{2-[(E)-1,2-difluoro-2-phenylvinyl]pyridin-4-yl}-1,5,6,7- tetrahydro-4H-pyrrolo[3,2-c]pyridin-4-one trifluoroacetate | 0.0305 | |
| 178 | 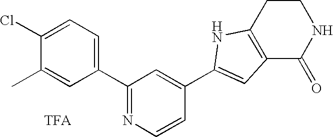 | 2-[2-(4-chloro-3-methylphenyl)pyridin-4-yl]-1,5,6,7-tetrahydro- 4H-pyrrolo[3,2-c]pyridin-4-one trifluoroacetate | 0.0306 | |
| 179 | 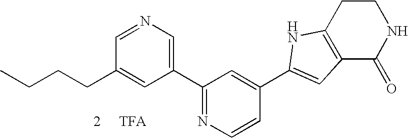 | 2-(5′-butyl-2,3′-bipyridin-4-yl)-1,5,6,7-tetrahydro-4H-pyrrolo[3,2- c]pyridin-4-one trifluoroacetate | 0.0309 | |
| 180 | 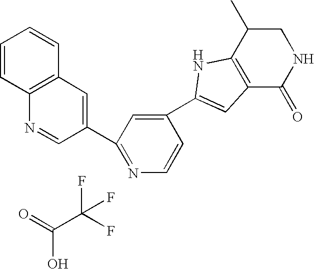 | 7-methyl-2-(2-quinolin-3-ylpyridin-4-yl)-1,5,6,7-tetrahydro-4H- pyrrolo[3,2-c]pyridin-4-one trifluoroacetate | 0.031 | |
| 181 | 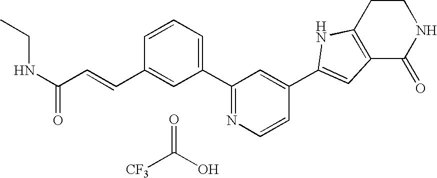 | (2E)-N-ethyl-3-{3-[4-(4-oxo-4,5,6,7-tetrahydro-1H-pyrrolo[3,2- c]pyridin-2-yl)pyridin-2-yl]phenyl}acrylamide trifluoroacetate | 0.0311 | |
| 182 | 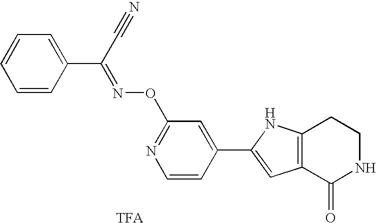 | (2Z)-({[4-(4-oxo-4,5,6,7-tetrahydro-1H-pyrrolo[3,2-c]pyridin-2- yl)pyridin-2-yl]oxy}imino)(phenyl)acetonitrile trifluoroacetate | 0.0313 | |
| 183 | 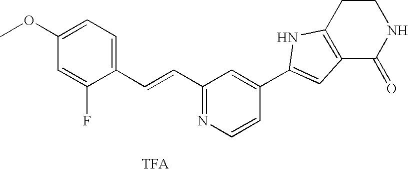 | 2-{2-[(E)-2-(2-fluoro-4-methoxyphenyl)vinyl]pyridin-4-yl}-1,5,6,7- tetrahydro-4H-pyrrolo[3,2-c]pyridin-4-one trifluoroacetate | 0.0314 | |
| 184 | 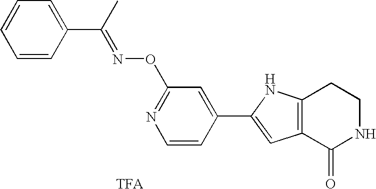 | 2-[2-({[(1E)-1-phenylethylidene]amino)oxy)pyridin-4-yl]-1,5,6,7- tetrahydro-4H-pyrrolo[3,2-c]pyridin-4-one trifluoroacetate | 0.0314 | |
| 185 | 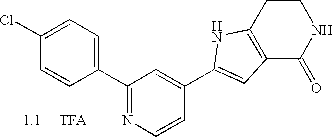 | 2-[2-(4-chlorophenyl)pyridin-4-yl]-1,5,6,7-tetrahydro-4H- pyrrolo[3,2-c]pyridin-4-one trifluoroacetate | 0.0315 | |
| 186 | 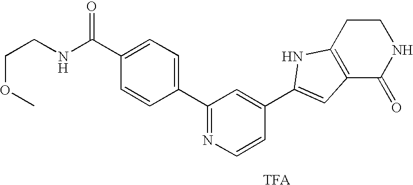 | N-(2-methoxyethyl)-4-[4-(4-oxo-4,5,6,7-tetrahydro-1H- pyrrolo[3,2-c]pyridin-2-yl)pyridin-2-yl]benzamide trifluoroacetate | 0.0315 | |
| 187 | 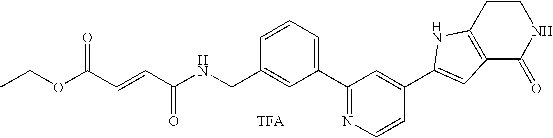 | ethyl (2E)-4-oxo-4-({3-[4-(4-oxo-4,5,6,7-tetrahydro-1H- pyrrolo[3,2-c]pyridin-2-yl)pyridin-2-yl]benzyl}amino)but-2-enoate trifluoroacetate | 0.0317 | |
| 188 | 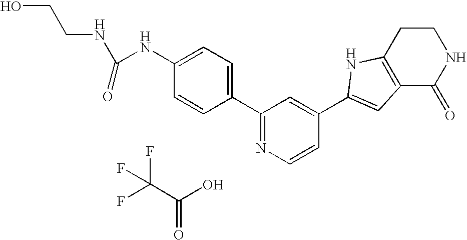 | N-(2-hydroxyethyl)-N′-{4-[4-(4-oxo-4,5,6,7-tetrahydro-1H- pyrrolo[3,2-c]pyridin-2-yl)pyridin-2-yl]phenyl}urea trifluoroacetate | 0.0317 | |
| 189 | 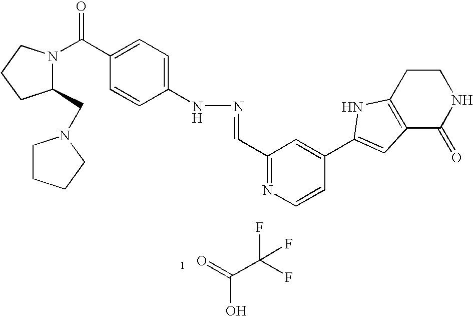 | 4-(4-oxo-4,5,6,7-tetrahydro-1H-pyrrolo[3,2-c]pyridin-2- yl)pyridine-2-carbaldehyde (4-{[(2R)-2-(pyrrolidin-1- ylmethyl)pyrrolidin-1-yl]carbonyl)phenyl)hydrazone trifluoroacetate | 0.0318 | |
| 190 | 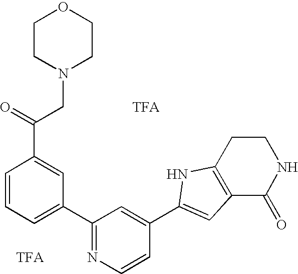 | 2-{2-[3-(morpholin-4-ylacetyl)phenyl]pyridin-4-yl}-1,5,6,7- tetrahydro-4H-pyrrolo[3,2-c]pyridin-4-one bis(trifluoroacetate) | 0.032 | |
| 191 | 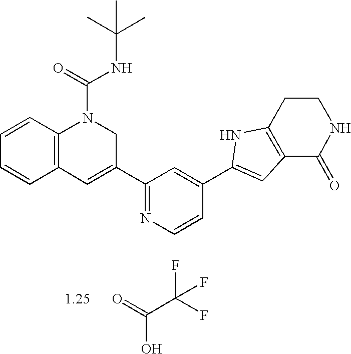 | N-(tert-butyl)-3-[4-(4-oxo-4,5,6,7-tetrahydro-1H-pyrrolo[3,2- c]pyridin-2-yl)pyridin-2-yl]quinoline-1(2H)-carboxamide trifluoroacetate | 0.0322 | |
| 192 | 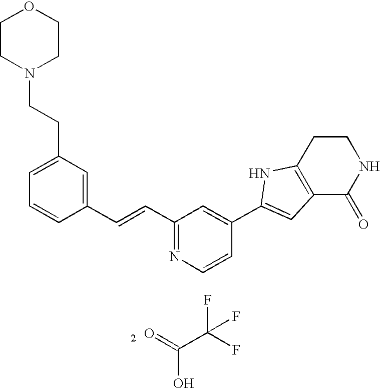 | 2-(2-{(E)-2-[3-(2-morpholin-4-ylethyl)phenyl]vinyl}pyridin-4-yl)- 1,5,6,7-tetrahydro-4H-pyrrolo[3,2-c]pyridin-4-one trifluoroacetate | 0.0324 | |
| 193 | 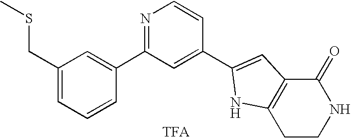 | 2-(2-{3-((methylthio)methyl]phenyl}pyridin-4-yl)-1,5,6,7- tetrahydro-4H-pyrrolo[3,2-c]pyridin-4-one trifluoroacetate | 0.0333 | |
| 194 | 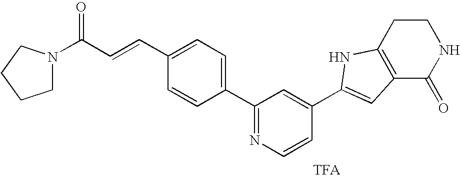 | 2-(2-{4-[(1E)-3-oxo-3-pyrrolidin-1-ylprop-1-enyl]phenyl}pyridin-4- yl)-1,5,6,7-tetrahydro-4H-pyrrolo[3,2-c]pyridin-4-one trifluoroacetate | 0.0334 | |
| 195 | 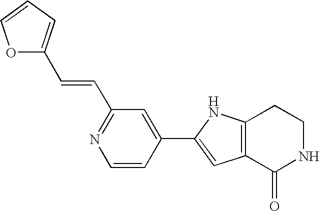 | 2-{2-[(E)-2-(2-furyl)vinyl]pyridin-4-yl}-1,5,6,7-tetrahydro-4H- pyrrolo[3,2-c]pyridin-4-one | 0.0335 | |
| 196 | 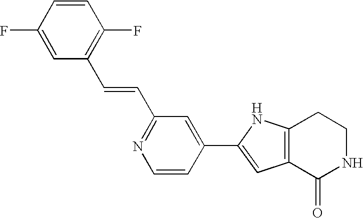 | 2-{2-[(E)-2-(2,5-difluorophenyl)vinyl]pyridin-4-yl}-1,5,6,7- tetrahydro-4H-pyrrolo[3,2-c]pyridin-4-one | 0.0336 | |
| 197 | 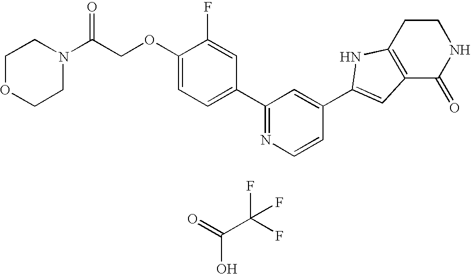 | 2-{2-[3-fluoro-4-(2-morpholin-4-yl-2-oxoethoxy)phenyl]pyridin-4- yl}-1,5,6,7-tetrahydro-4H-pyrrolo[3,2-c]pyridin-4-one trifluoroacetate | 0.034 | |
| 198 | 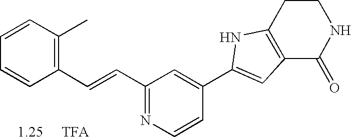 | 2-{2-[(E)-2-(2-methylphenyl)vinyl]pyridin-4-yl}-1,5,6,7-tetrahydro- 4H-pyrrolo[3,2-c]pyridin-4-one trifluoroacetate | 0.0345 | |
| 199 | 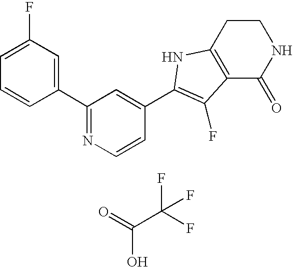 | 3-fluoro-2-[2-(3-fluorophenyl)pyridin-4-yl]-1,5,6,7-tetrahydro-4H- pyrrolo[3,2-c]pyridin-4-one trifluoroacetate | 0.0348 | |
| 200 | 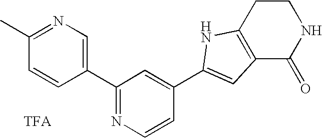 | 2-(6′-methyl-2,3′-bipyridin-4-yl)-1,5,6,7-tetrahydro-4H- pyrrolo[3,2-c]pyridin-4-one trifluoroacetate | 0.0348 | |
| 201 | 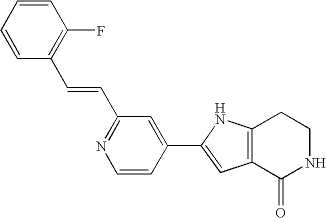 | 2-{2-[(E)-2-(2-fluorophenyl)vinyl]pyridin-4-yl}-1,5,6,7-tetrahydro- 4H-pyrrolo[3,2-c]pyridin-4-one | 0.0348 | |
| 202 | 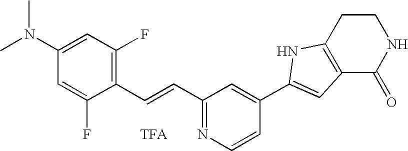 | 2-(2-{(E)-2-[4-(dimethylamino)-2,6-difluorophenyl]vinyl}pyridin-4- yl)-1,5,6,7-tetrahydro-4H-pyrrolo[3,2-c]pyridin-4-one trifluoroacetate | 0.0348 | |
| 203 | 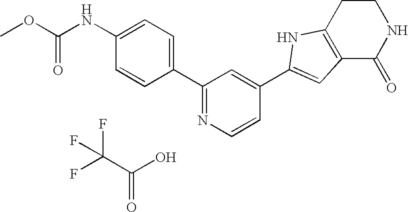 | methyl 4-[4-(4-oxo-4,5,6,7-tetrahydro-1H-pyrrolo[3,2-c]pyridin-2- yl)pyridin-2-yl]phenylcarbamate trifluoroacetate | 0.0351 | |
| 204 | 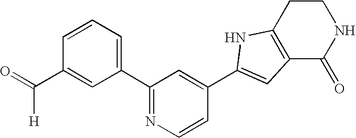 | 3-[4-(4-oxo-4,5,6,7-tetrahydro-1H-pyrrolo[3,2-c]pyridin-2- yl)pyridin-2-yl]benzaldehyde | 0.0355 | |
| 205 | 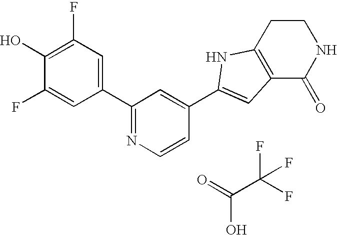 | 2-[2-(3,5-difluoro-4-hydroxyphenyl)pyridin-4-yl]-1,5,6,7- tetrahydro-4H-pyrrolo[3,2]pyridin-4-one trifluoroacetate | 0.0355 | |
| 206 | 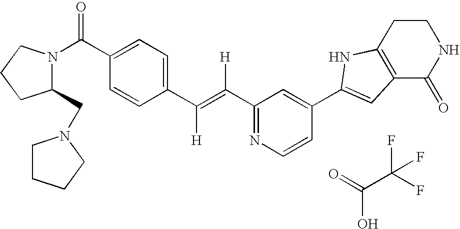 | 2-{2-[(E)-2-(4-{[(2R)-2-(pyrrolidin-1-ylmethyl)pyrrolidin-1- yl]carbonyl)phenyl)vinyl]pyridin-4-yl}-1,5,6,7-tetrahydro-4H- pyrrolo[3,2-c]pyridin-4-one trifluoroacetate | 0.0355 | |
| 207 | 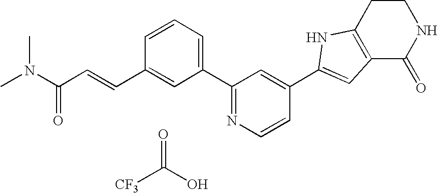 | (2E)-N,N-dimethyl-3-{3-[4-(4-oxo-4,5,6,7-tetrahydro-1H- pyrrolo[3,2-c]pyridin-2-yl)pyridin-2-yl]phenyl}acrylamide trifluoroacetate | 0.0356 | |
| 208 | 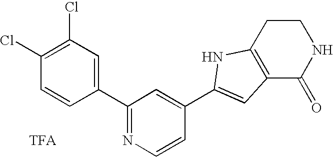 | 2-[2-(3,4-dichlorophenyl)pyridin-4-yl]-1,5,6,7-tetrahydro-4H- pyrrolo[3,2-c]pyridin-4-one trifluoroacetate | 0.0358 | |
| 209 | 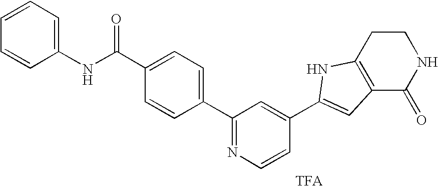 | 4-[4-(4-oxo-4,5,6,7-tetrahydro-1H-pyrrolo[3,2-c]pyridin-2- yl)pyridin-2-yl]-N-phenylbenzamide trifluoroacetate | 0.0359 | |
| 210 | 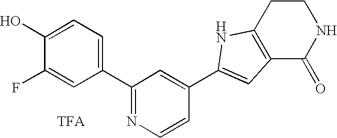 | 2-[2-(3-fluoro-4-hydroxyphenyl)pyridin-4-yl]-1,5,6,7-tetrahydro- 4H-pyrrolo[3,2-c]pyridin-4-one trifluoroacetate | 0.036 | |
| 211 | 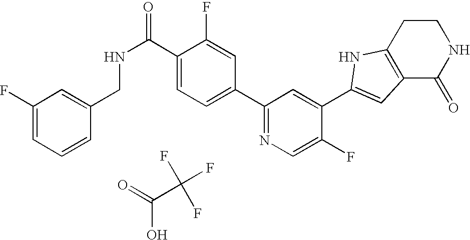 | 2-fluoro-N-(3-fluorobenzyl)-4-[5-fluoro-4-(4-oxo-4,5,6,7- tetrahydro-1H-pyrrolo[3,2-c]pyridin-2-yl)pyridin-2-yl]benzamide trifluoroacetate | 0.0362 | |
| 212 | 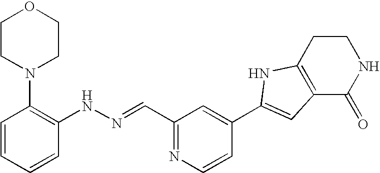 | 4-(4-oxo-4,5,6,7-tetrahydro-1H-pyrrolo[3,2-c]pyridin-2- yl)pyridine-2-carbaldehyde (2-morpholin-4-ylphenyl)hydrazone | 0.0366 | |
| 213 | 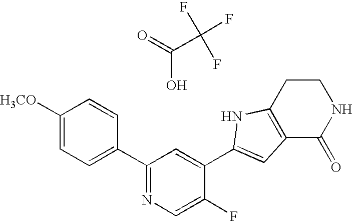 | 2-[5-fluoro-2-(4-methoxyphenyl)pyridin-4-yl]-1,5,6,7-tetrahydro- 4H-pyrrolo[3,2-c]pyridin-4-one trifluoroacetate | 0.0367 | |
| 214 | 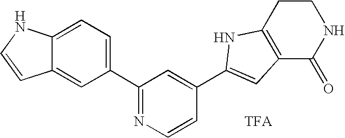 | 2-[2-(1H-indol-5-yl)pyridin-4-yl]-1,5,6,7-tetrahydro-4H- pyrrolo[3,2-c]pyridin-4-one trifluoroacetate | 0.0369 | |
| 215 | 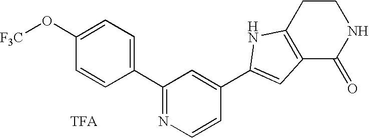 | 2-{2-[4-(trifluoromethoxy)phenyl]pyridin-4-yl}-1,5,6,7-tetrahydro- 4H-pyrrolo[3,2-c]pyridin-4-one trifluoroacetate | 0.0371 | |
| 216 | 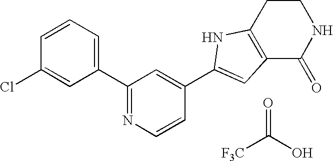 | 2-[2-(3-chlorophenyl)pyridin-4-yl]-1,5,6,7-tetrahydro-4H- pyrrolo[3,2-c]pyridin-4-one trifluoroacetate | 0.0371 | |
| 217 | 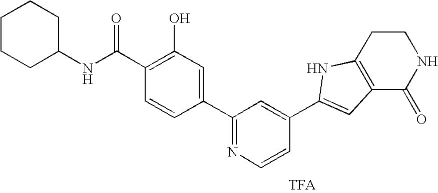 | N-cyclohexyl-2-hydroxy-4-[4-(4-oxo-4,5,6,7-tetrahydro-1H- pyrrolo[3,2-c]pyridin-2-yl)pyridin-2-yl]benzamide trifluoroacetate | 0.0371 | |
| 218 | 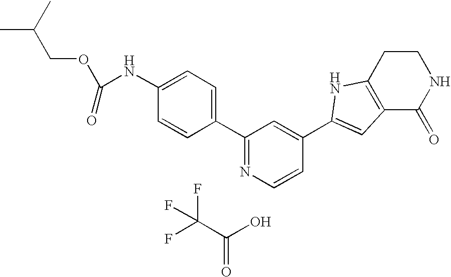 | isobutyl 4-[4-(4-oxo-4,5,6,7-tetrahydro-1H-pyrrolo[3,2-c]pyridin- 2-yl)pyridin-2-yl]phenylcarbamate trifluoroacetate | 0.0374 | |
| 219 | 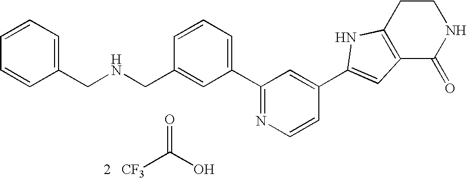 | 2-(2-{3-[(benzylamino)methyl]phenyl}pyridin-4-yl)-1,5,6,7- tetrahydro-4H-pyrrolo[3,2-c]pyridin-4-one trifluoroacetate | 0.0377 | |
| 220 | 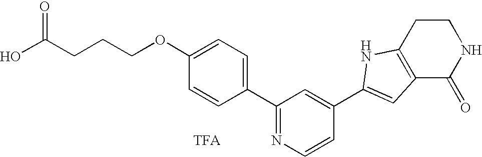 | 4-{4-[4-(4-oxo-4,5,6,7-tetrahydro-1H-pyrrolo[3,2-c]pyridin-2- yl)pyridin-2-yl]phenoxy}butanoic acid trifluoroacetate | 0.0379 | |
| 221 | 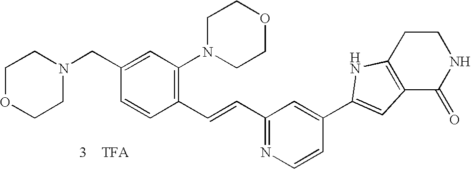 | 2-(2-{(E)-2-[2-morpholin-4-yl-4-(morpholin-4- ylmethyl)phenyl]vinyl}pyridin-4-yl)-1,5,6,7-tetrahydro-4H- pyrrolo[3,2-c]pyridin-4-one trifluoroacetate | 0.038 | |
| 222 | 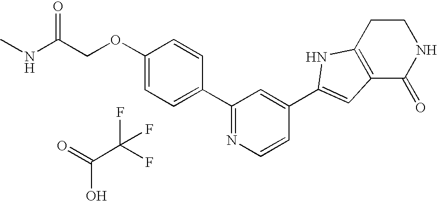 | N-methyl-2-{4-[4-(4-oxo-4,5,6,7-tetrahydro-1H-pyrrolo[3,2- c]pyridin-2-yl)pyridin-2-yl]phenoxy)acetamide trifluoroacetate | 0.0384 | |
| 223 | 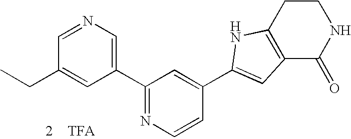 | 2-(5′-ethyl-2,3′-bipyridin-4-yl)-1,5,6,7-tetrahydro-4H-pyrrolo[3,2- c]pyridin-4-one trifluoroacetate | 0.0386 | |
| 224 | 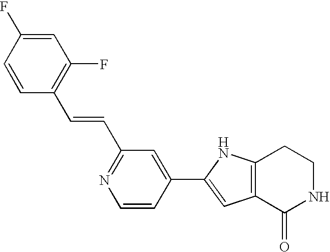 | 2-{2-[(E)-2-(2,4-difluorophenyl)ethenyl]pyridin-4-yl}-1,5,6,7- tetrahydro-4H-pyrrolo[3,2-c]pyridin-4-one | 0.0386 | |
| 225 | 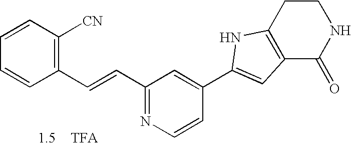 | 2-{(E)-2-[4-(4-oxo-4,5,6,7-tetrahydro-1H-pyrrolo[3,2-c]pyridin-2- yl)pyridin-2-yl]vinyl}benzonitrile trifluoroacetate | 0.0389 | |
| 226 | 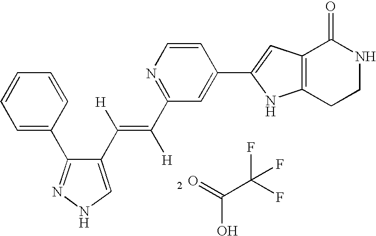 | 2-{2-[(E)-2-(3-phenyl-1H-pyrazol-4-yl)vinyl]pyridin-4-yl)-1,5,6,7- tetrahydro-4H-pyrrolo[3,2-c]pyridin-4-one trifluoroacetate | 0.0392 | |
| 227 | 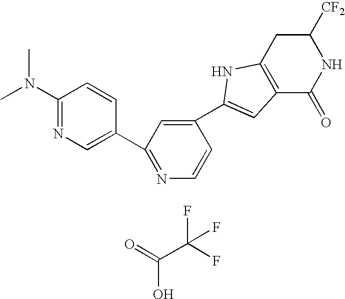 | 2-[6′-(dimethylamino)-2,3′-bipyridin-4-yl]-6-(trifluoromethyl)- 1,5,6,7-tetrahydro-4H-pyrrolo[3,2-c]pyridin-4-one trifluoroacetate | 0.0393 | |
| 228 | 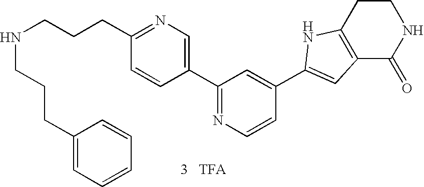 | 2-(6′-{3-[(3-phenylpropyl)amino]propyl}-2,3-bipyridin-4-yl}- 1,5,6,7-tetrahydro-4H-pyrrolo[3,2-c]pyridin-4-one trifluoroacetate | 0.0393 | |
| 229 | 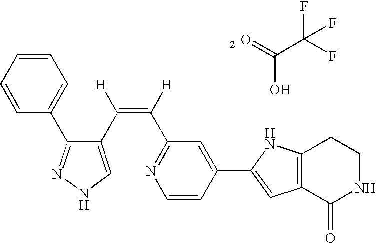 | 2-{2-[(Z)-2-(3-phenyl-1H-pyrazol-4-yl)vinyl]pyridin-4-yl}-1,5,6,7- tetrahydro-4H-pyrrolo[3,2-c]pyridin-4-one trifluoroacetate | 0.0393 | |
| 230 | 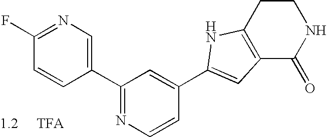 | 2-(6′-fluoro-2,3′-bipyridin-4-yl)-1,5,6,7-tetrahydro-4H-pyrrolo[3,2- c]pyridin-4-one trifluoroacetate | 0.0394 | |
| 231 | 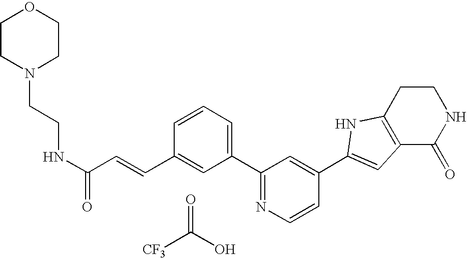 | (2E)-N-(2-morpholin-4-ylethyl)-3-{3-[4-(4-oxo-4,5,6,7-tetrahydro- 1H-pyrrolo[3,2-c]pyridin-2-yl)pyridin-2-yl]phenyl}acrylamide trifluoroacetate | 0.0396 | |
| 232 | 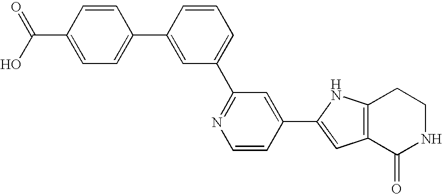 | 3′-[4-(4-oxo-4,5,6,7-tetrahydro-1H-pyrrolo[3,2-c]pyridin-2- yl)pyridin-2-yl]-1,1′-biphenyl-4-carboxylic acid | 0.0397 | |
| 233 | 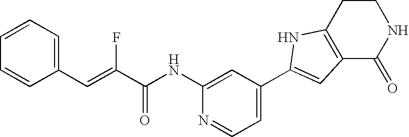 | (2Z)-2-fluoro-N-[4-(4-oxo-4,5,6,7-tetrahydro-1H-pyrralo[3,2- c]pyridin-2-yl)pyridin-2-yl]-3-phenylacrylamide | 0.0399 | |
| 234 | 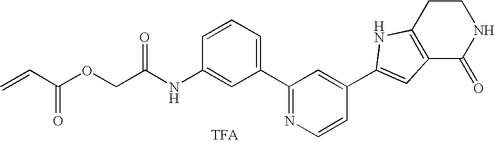 | 2-oxo-2-({3-[4-(4-oxo-4,5,6,7-tetrahydro-1H-pyrrolo[3,2- c]pyridin-2-yl)pyridin-2-yl]phenyl}amino)ethyl acrylate trifluoroacetate | 0.0402 | |
| 235 | 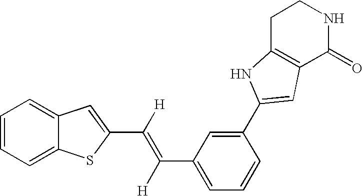 | 2-{2-[(E)-2-(1-benzothien-2-yl)vinyl]pyridin-4-yl)-1,5,6,7- tetrahydro-4H-pyrrolo[3,2-c]pyridin-4-one | 0.0402 | |
| 236 | 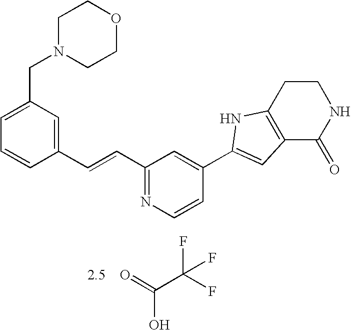 | 2-(2-{(E)-2-[3-(morpholin-4-ylmethyl)phenyl]vinyl}pyridin-4-yl)- 1,5,6,7-tetrahydro-4H-pyrrolo[3,2-c]pyridin-4-one trifluoroacetate | 0.0403 | |
| 237 | 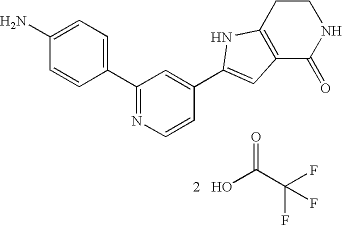 | 2-[2-(4-aminophenyl)pyridin-4-yl]-1,5,6,7-tetrahydro-4H- pyrrolo[3,2-c]pyridin-4-one trifluoroacetate | 0.0404 | |
| 238 | 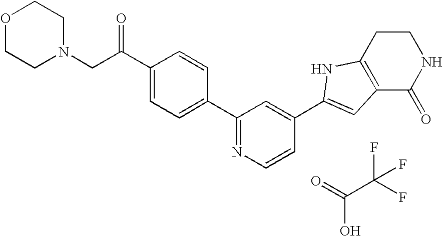 | 2-{2-[4-(morpholin-4-ylacetyl)phenyl]pyridin-4-yl}-1,5,6,7- tetrahydro-4H-pyrrolo[3,2-c]pyridin-4-one trifluoroacetate | 0.041 | |
| 239 | 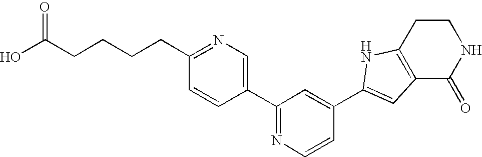 | 5-[4-(4-oxo-4,5,6,7-tetrahydro-1H-pyrrolo[3,2-c]pyridin-2-yl)-2,3′- bipyridin-6′-yl]pentanoic acid | 0.041 | |
| 240 | 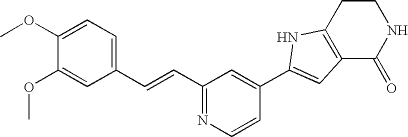 | 2-{2-[(E)-2-(3,4-dimethoxyphenyl)vinyl]pyridin-4-yl}-1,5,6,7- tetrahydro-4H-pyrrolo[3,2-c]pyridin-4-one | 0.0414 | |
| 241 | 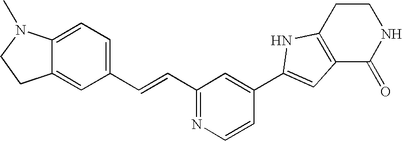 | 2-{2-[(E)-2-(1-methyl-2,3-dihydro-1H-indol-5-yl)vinyl]pyridin-4- yl}-1,5,6,7-tetrahydro-4H-pyrrolo[3,2-c]pyridin-4-one | 0.0415 | |
| 242 | 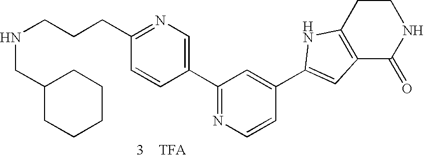 | 2-(6′-{3-[(cyclohexylmethyl)amino]propyl}-2,3′-bipyridin-4-yl)- 1,5,6,7-tetrahydro-4H-pyrrolo[3,2-c]pyridin-4-one trifluoroacetate | 0.0417 | |
| 243 | 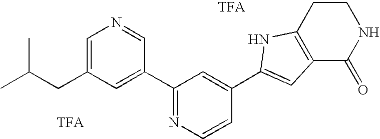 | 2-(5′-isobutyl-2,3-bipyridin-4-yl)-1,5,6,7-tetrahydro-4H- pyrrolo[3,2-c]pyridin-4-one bis(trifluoroacetate) | 0.0418 | |
| 244 | 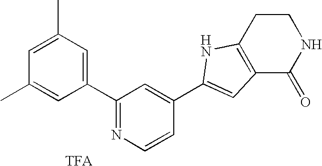 | 2-[2-(3,5-dimethylphenyl)pyridin-4-yl]-1,5,6,7-tetrahydro-4H- pyrrolo[3,2-c]pyridin-4-one trifluoroacetate | 0.0419 | |
| 245 | 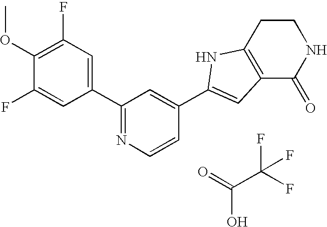 | 2-[2-(3,5-difluoro-4-methoxyphenyl)pyridin-4-yl]-1,5,6,7- tetrahydro-4H-pyrrolo[3,2-c]pyridin-4-one trifluoroacetate | 0.042 | |
| 246 | 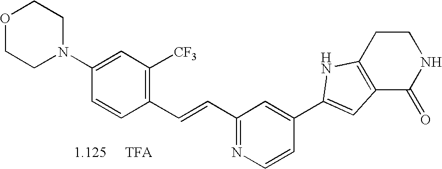 | 2-(2-{(E)-2-(4-morpholin-4-yl-2- (trifluoromethyl)phenyl]vinyl}pyridin-4-yl)-1,5,6,7-tetrahydro-4H- pyrrolo[3,2-c]pyridin-4-one trifluoroacetate | 0.0421 | |
| 247 | 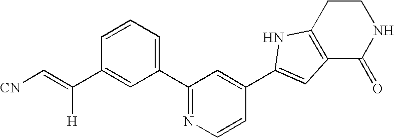 | (2E)-3-{3-[4-(4-oxo-4,5,6,7-tetrahydro-1H-pyrrolo[3,2-c]pyridin-2- yl)pyridin-2-yl]phenyl}acrylonitrile | 0.0424 | |
| 248 | 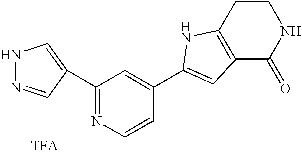 | 2-[2-(1H-pyrazol-4-yl)pyridin-4-yl]-1,5,6,7-tetrahydro-4H- pyrrolo[3,2-c]pyridin-4-one trifluoroacetate | 0.0425 | |
| 249 | 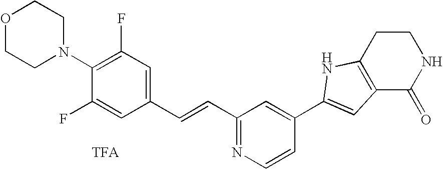 | 2-{2-[(E)-2-(3,5-difluoro-4-morpholin-4-ylphenyl)vinyl]pyridin-4- yl}-1,5,6,7-tetrahydro-4H-pyrrolo[3,2-c]pyridin-4-one trifluoroacetate | 0.0441 | |
| 250 | 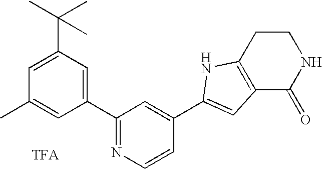 | 2-[2-(3-tert-butyl-5-methylphenyl)pyridin-4-yl]-1,5,6,7-tetrahydro- 4H-pyrrolo[3,2-c]pyridin-4-one trifluoroacetate | 0.0442 | |
| 251 | 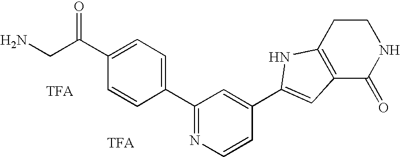 | 2-{2-[4-(aminoacetyl)phenyl]pyridin-4-yl}-1,5,6,7-tetrahydro-4H- pyrrolo[3,2-c]pyridin-4-one bis(trifluoroacetate) | 0.0443 | |
| 252 | 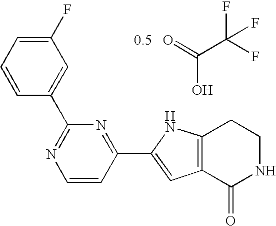 | 2-[2-(3-fluorophenyl)pyrimidin-4-yl]-1,5,6,7-tetrahydro-4H- pyrrolo[3,2-c]pyridin-4-one trifluoroacetate | 0.0445 | |
| 253 | 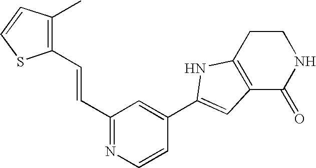 | 2-{2-[(E)-2-(3-methylthien-2-yl)vinyl]pyridin-4-yl}-1,5,6,7- tetrahydro-4H-pyrrolo[3,2-c]pyridin-4-one | 0.0449 | |
| 254 | 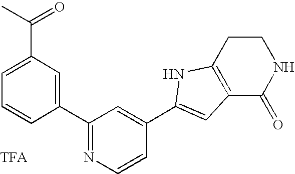 | 2-[2-(3-acetylphenyl)pyridin-4-yl]-1,5,6,7-tetrahydro-4H- pyrrolo[3,2-c]pyridin-4-one trifluoroacetate | 0.045 | |
| 255 | 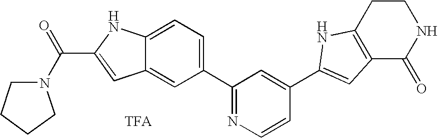 | 2-{2-[2-(pyrrolidin-1-ylcarbonyl)-1H-indol-5-yl]pyridin-4-yl)- 1,5,6,7-tetrahydro-4H-pyrrolo[3,2-c]pyridin-4-one trifluoroacetate | 0.0454 | |
| 256 | 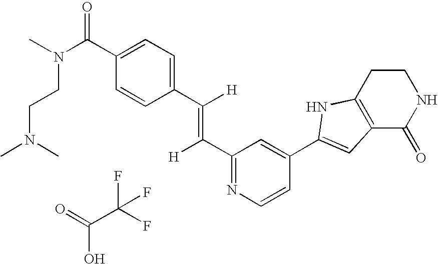 | N-(2-(dimethylamino)ethyl]-N-methyl-4-{(E)-2-[4-(4-oxo-4,5,6,7- tetrahydro-1H-pyrrolo[32-c]pyridin-2-yl)pyridin-2- yl]vinyl}benzamide trifluoroacetate | 0.0454 | |
| 257 | 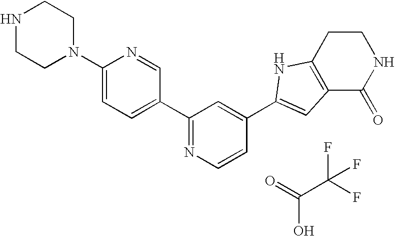 | 2-(6′-piperazin-1-yl-2,3′-bipyridin-4-yl)-1,5,6,7-tetrahydro-4H- pyrrolo[3,2-c]pyridin-4-one trifluoroacetate | 0.0455 | |
| 258 | 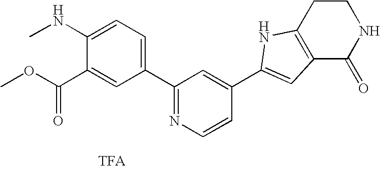 | methyl 2-(methylamino)-5-[4-(4-oxo-4,5,6,7-tetrahydro-1H- pyrrolo[3,2-c]pyridin-2-yl)pyridin-2-yl]benzoate trifluoroacetate | 0.0455 | |
| 259 | 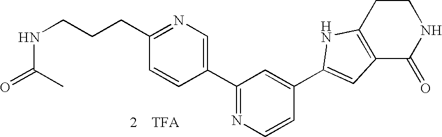 | N-{3-[4-(4-oxo-4,5,6,7-tetrahydro-1H-pyrrolo[3,2-c]pyridin-2-yl)- 2,3′-bipyridin-6′-yl]propyl}acetamide trifluoroacetate | 0.0455 | |
| 260 | 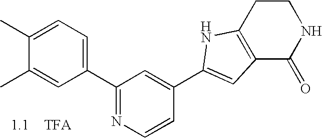 | 2-[2-(3,4-dimethylphenyl)pyridin-4-yl]-1,5,6,7-tetrahydro-4H- pyrrolo[3,2-c]pyridin-4-one trifluoroacetate | 0.0456 | |
| 261 | 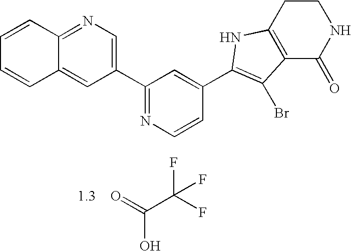 | 3-bromo-2-(2-quinolin-3-ylpyridin-4-yl)-1,5,6,7-tetrahydro-4H- pyrrolo[3,2-c]pyridin-4-one trifluoroacetate | 0.0459 | |
| 262 | 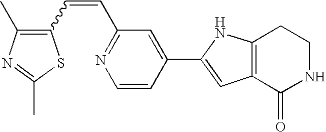 | 2-{2-[(Z)-2-(2,4-dimethyl-1,3-thiazol-5-yl)vinyl]pyridin-4-yl)- 1,5,6,7-tetrahydro-4H-pyrrolo[3,2-c]pyridin-4-one | 0.0459 | |
| 263 | 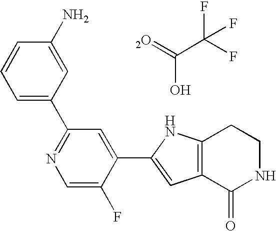 | 2-[2-(3-aminophenyl)-5-fluoropyridin-4-yl]-1,5,6,7-tetrahydro-4H- pyrrolo[3,2-c]pyridin-4-one trifluoroacetate | 0.046 | |
| 264 | 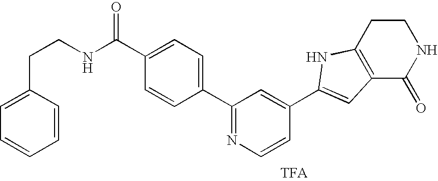 | 4-[4-(4-oxo-4,5,6,7-tetrahydro-1H-pyrrolo[3,2-c]pyridin-2- yl)pyridin-2-yl]-N-(2-phenylethyl)benzamide trifluoroacetate | 0.0461 | |
| 265 | 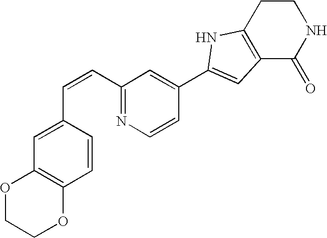 | 2-{2-[(Z)-2-(2,3-dihydro-1,4-benzodioxin-6-yl)vinyl]pyridin-4-yl}- 1,5,6,7-tetrahydro-4H-pyrrolo[3,2-c]pyridin-4-one | 0.047 | |
| 266 | 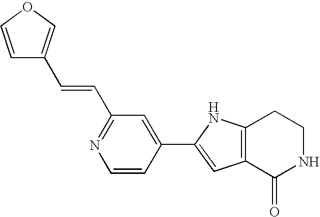 | 2-{2-[(E)-2-(3-furyl)vinyl]pyridin-4-yl}-1,5,6,7-tetrahydro-4H- pyrrolo[3,2-c]pyridin-4-one | 0.0477 | |
| 267 | 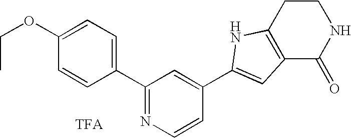 | 2-[2-(4-ethoxyphenyl)pyridin-4-yl]-1,5,6,7-tetrahydro-4H- pyrrolo[3,2c]pyridin-4-one trifluoroacetate | 0.0478 | |
| 268 | 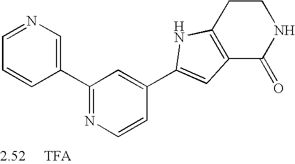 | 2-(2,3′-bipyridin-4-yl)-1,5,6,7-tetrahydro-4H-pyrrolo[3,2-c]pyridin- 4-one trifluoroacetate | 0.0479 | |
| 269 | 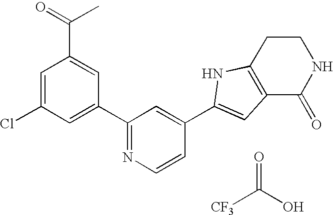 | 2-[2-(3-acetyl-5-chlorophenyl)pyridin-4-yl]-1,5,6,7-tetrahydro-4H- pyrrolo(3,2-c]pyridin-4-one trifluoroacetate | 0.0479 | |
| 270 | 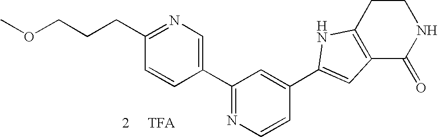 | 2-[6′-(3-methoxypropyl)-2,3′-bipyridin-4-yl]-1,5,6,7-tetrahydro- 4H-pyrrolo[3,2-c]pyridin-4-one trifluoroacetate | 0.0481 | |
| 271 | 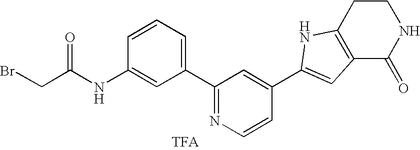 | 2-bromo-N-{3-[4-(4-oxo-4,5,6,7-tetrahydro-1H-pyrrolo[3,2- c]pyridin-2-yl)pyridin-2-yl]phenyl}acetamide trifluoroacetate | 0.0493 | |
| 272 | 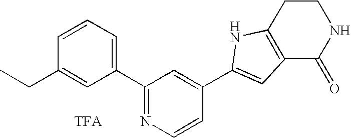 | 2-[2-(3-ethylphenyl)pyridin-4-yl]-1,5,6,7-tetrahydro-4H- pyrrolo[3,2-c]pyridin-4-one trifluoroacetate | 0.0493 | |
| 273 | 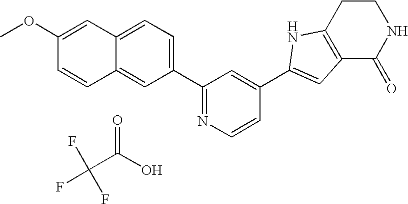 | 2-[2-(6-methoxy-2-naphthyl)pyridin-4-yl]-1,5,6,7-tetrahydro-4H- pyrrolo[3,2-c]pyridin-4-one trifluoroacetate | 0.0497 | |
| 274 | 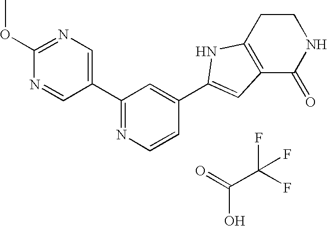 | 2-[2-(2-methoxypyrimidin-5-yl)pyridin-4-yl]-1,5,6,7-tetrahydro- 4H-pyrrolo[3,2-c]pyridin-4-one trifluoroacetate | 0.0501 | |
| 275 | 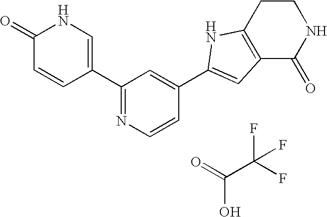 | 4-(4-oxo-4,5,6,7-tetrahydro-1H-pyrrolo[3,2-c]pyridin-2-yl)-2,3′- bipyridin-6′(1′H)-one trifluoroacetate | 0.0501 | |
| 276 | 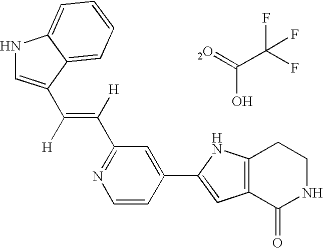 | 2-{2-[(E)-2-(1H-indol-3-yl)ethenyl]pyridin-4-yl}-1,5,6,7-tetrahydro- 4H-pyrrolo[3,2-c]pyridin-4-one trifluoroacetate | 0.0501 | |
| 277 | 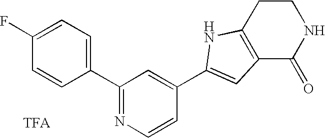 | 2-[2-(4-fluorophenyl)pyridin-4-yl]-1,5,6,7-tetrahydro-4H- pyrrolo[3,2-c]pyridin-4-one trifluoroacetate | 0.0502 | |
| 278 | 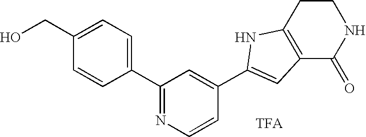 | 2-{2-[4-(hydroxymethyl)phenyl]pyridin-4-yl}-1,5,6,7-tetrahydro- 4H-pyrrolo[3,2-c]pyridin-4-one trifluoroacetate | 0.0504 | |
| 279 | 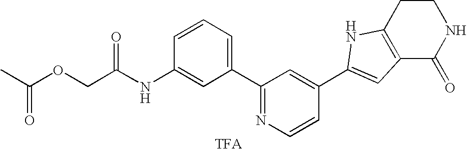 | 2-oxo-2-({3-[4-(4-oxo-4,5,6,7-tetrahydro-1H-pyrrolo[3,2- c]pyridin-2-yl)pyridin-2-yl]phenyl}amino)ethyl acetate trifluoroacetate | 0.0511 | |
| 280 | 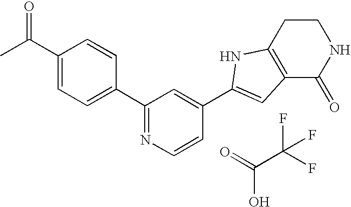 | 2-[2-(4-acetylphenyl)pyridin-4-yl]-1,5,6,7-tetrahydro-4H- pyrrolo[3,2-c]pyridin-4-one trifluoroacetate | 0.0512 | |
| 281 | 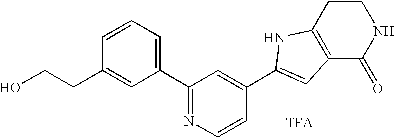 | 2-{2-[3-(2-hydroxyethyl)phenyl]pyridin-4-yl}-1,5,6,7-tetrahydro- 4H-pyrrolo[3,2-c]pyridin-4-one trifluoroacetate | 0.0514 | |
| 282 | 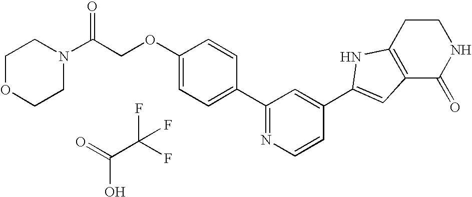 | 2-{2-[4-(2-morpholin-4-yl-2-oxoethoxy)phenyl]pyridin-4-yl}- 1,5,6,7-tetrahydro-4H-pyrralo[3,2-c]pyridin-4-one trifluoroacetate | 0.0514 | |
| 283 | 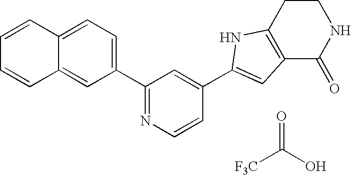 | 2-[2-(2-naphthyl)pyridin-4-yl]-1,5,6,7-tetrahydro-4H-pyrrolo[3,2- c]pyridin-4-one trifluoroacetate | 0.052 | |
| 284 | 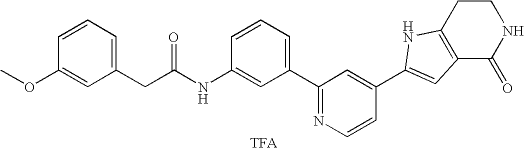 | 2-(3-methoxyphenyl)-N-{3-[4-(4-oxo-4,5,6,7-tetrahydro-1H- pyrrolo[3,2-c]pyridin-2-yl)pyridin-2-yl]phenyl}acetamide trifluoroacetate | 0.052 | |
| 285 | 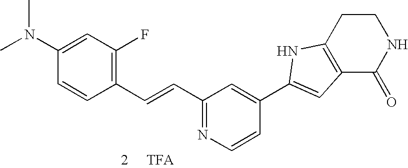 | 2-(2-{(E)-2-[4-(dimethylamino)-2-fluorophenyl]vinyl}pyridin-4-yl)- 1,5,6,7-tetrahydro-4H-pyrrolo[3,2-c]pyridin-4-one trifluoroacetate | 0.0521 | |
| 286 | 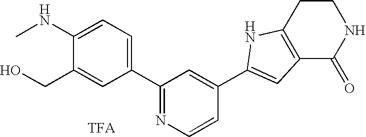 | 2-{2-[3-(hydroxymethyl)-4-(methylamino)phenyl]pyridin-4-yl}- 1,5,6,7-tetrahydro-4H-pyrrolo[3,2-c]pyridin-4-one trifluoroacetate | 0.0523 | |
| 287 | 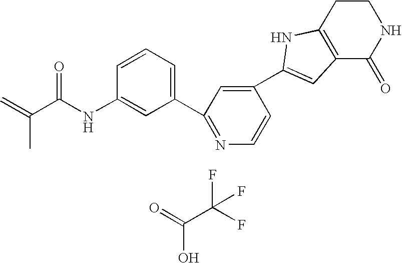 | 2-methyl-N-{3-[4-(4-oxo-4,5,6,7-tetrahydro-1H-pyrrolo[3,2- c]pyridin-2-yl)pyridin-2-yl]phenyl}acrylamide trifluoroacetate | 0.0525 | |
| 288 | 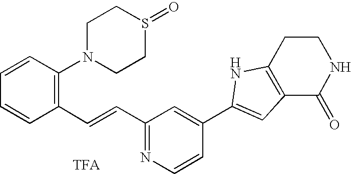 | 2-(2-{(E)-2-[2-(1-oxidothiomorphohn-4-yl)phenyl]vinyl}pyridin-4- yl)-1,5,6,7-tetrahydro-4H-pyrrolo[3,2-c]pyridin-4-one trifluoroacetate | 0.0527 | |
| 289 | 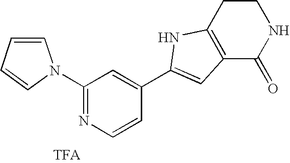 | 2-[2-(1H-pyrrol-1-yl)pyridin-4-yl]-1,5,6,7-tetrahydro-4H- pyrrolo[3,2-c]pyridin-4-one trifluoroacetate | 0.0528 | |
| 290 | 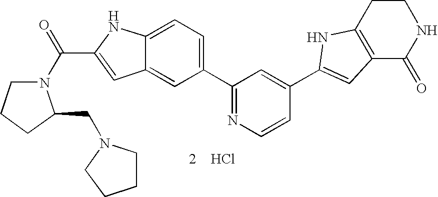 | 2-[2-(2-{[(2R)-2-(pyrrolidin-1-ylmethyl)pyrrolidin-1-yl]carbonyl)- 1H-indol-5-yl)pyridin-4-yl]-1,5,6,7-tetrahydro-4H-pyrrolo[3,2- c]pyridin-4-one hydrochloride | 0.0528 | |
| 291 | 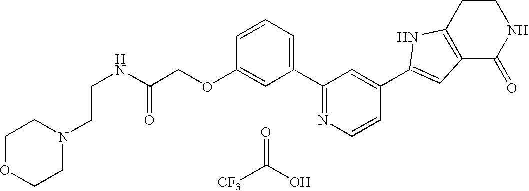 | N-(2-morpholin-4-ylethyl)-2-{3-[4-(4-oxo-4,5,6,7-tetrahydro-1H- pyrrolo[3,2-c]pyridin-2-yl)pyridin-2-yl]phenoxy}acetamide trifluoroacetate | 0.0529 | |
| 292 | 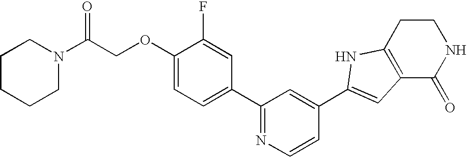 | 2-{2-[3-fluoro-4-(2-oxo-2-piperidin-1-ylethoxy)phenyl]pyridin-4- yl}-1,5,6,7-tetrahydro-4H-pyrrolo[3,2-c]pyridin-4-one | 0.0529 | |
| 293 | 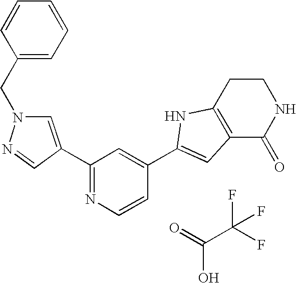 | 2-[2-(1-benzyl-1H-pyrazol-4-yl)pyridin-4-yl]-1,5,6,7-tetrahydro- 4H-pyrrolo[3,2-c]pyridin-4-one trifluoroacetate | 0.0531 | |
| 294 | 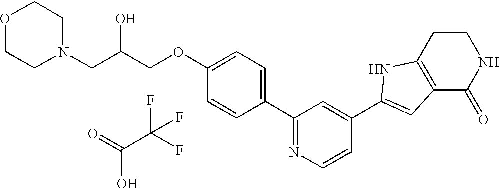 | 2-{2-[4-(2-hydroxy-3-morpholin-4-ylpropoxy)phenyl]pyridin-4-yl}- 1,5,6,7-tetrahydro-4H-pyrrolo[3,2-c]pyridin-4-one trifluoroacetate | 0.0532 | |
| 295 | 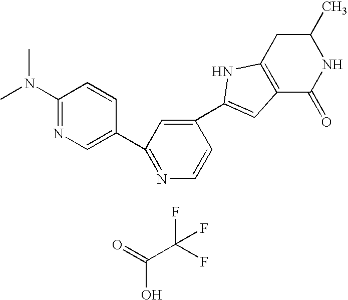 | 2-[6′-(dimethylamino)-2,3′-bipyridin-4-yl]-6-methyl-1,5,6,7- tetrahydro-4H-pyrrolo[3,2-c]pyridin-4-one trifluoroacetate | 0.0534 | |
| 296 | 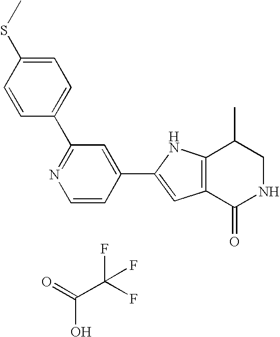 | 7-methyl-2-{2-[4-(methylthio)phenyl]pyridin-4-yl)-1,5,6,7- tetrahydro-4H-pyrrolo[3,2-c]pyridin-4-one trifluoroacetate | 0.0537 | |
| 297 | 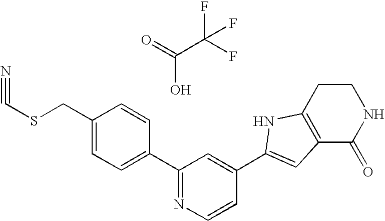 | 4-[4-(4-oxo-4,5,6,7-tetrahydro-1H-pyrrolo[3,2-c]pyridin-2- yl)pyridin-2-yl]benzyl thiocyanate trifluoroacetate | 0.0542 | |
| 298 | 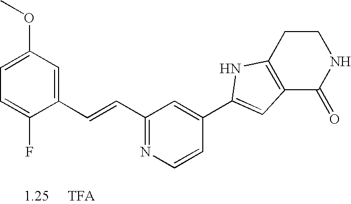 | 2-{2-[(E)-2-(2-fluoro-5-methoxyphenyl)vinyl]pyridin-4-yl}-1,5,6,7- tetrahydro-4H-pyrrolo[3,2-c]pyridin-4-one trifluoroacetate | 0.0542 | |
| 299 | 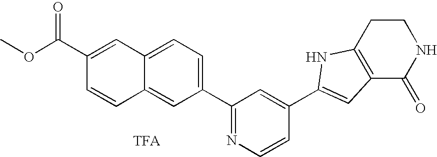 | methyl 6-[4-(4-oxo-4,5,6,7-tetrahydro-1H-pyrrolo(3,2-c]pyridin-2- yl)pyridin-2-yl]-2-naphthoate trifluoroacetate | 0.0547 | |
| 300 | 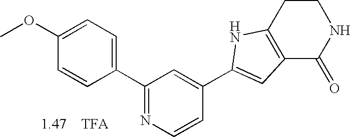 | 2-[2-(4-methoxyphenyl)pyridin-4-yl]-1,5,6,7-tetrahydro-4H- pyrrolo[3,2-c]pyridin-4-one trifluoroacetate | 0.0551 | |
| 301 | 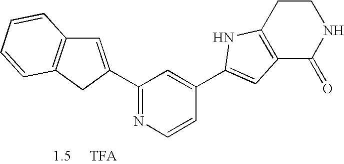 | 2-[2-(1H-inden-2-yl)pyridin-4-yl]-1,5,6,7-tetrahydro-4H- pyrrolo[3,2-c]pyridin-4-one trifluoroacetate | 0.0551 | |
| 302 | 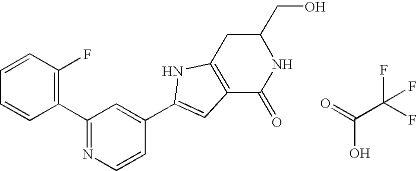 | 2-[2-(2-fluorophenyl)pyridin-4-yl]-6-(hydroxymethyl)-1,5,6,7- tetrahydro-4H-pyrrolo[3,2-c]pyridin-4-one trifluoroacetate | 0.0553 | |
| 303 | 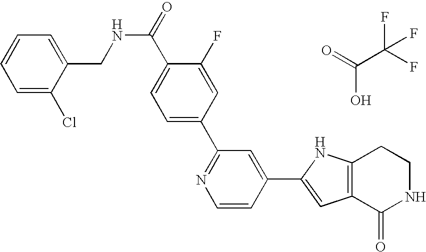 | N-(2-chlorobenzyl)-2-fluoro-4-[4-(4-oxo-4,5,6,7-tetrahydro-1H- pyrrolo[3,2-c]pyridin-2-yl)pyridin-2-yl]benzamide trifluoroacetate | 0.0555 | |
| 304 | 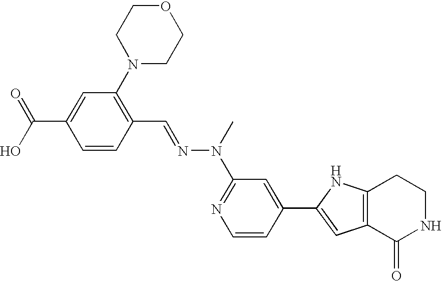 | 4-((E)-{methyl[4-(4-oxo-4,5,6,7-tetrahydro-1H-pyrrolo[3,2- c]pyridin-2-yl)pyridin-2-yl]hydrazono}methyl)-3-morpholin-4- ylbenzoic acid | 0.0556 | |
| 305 | 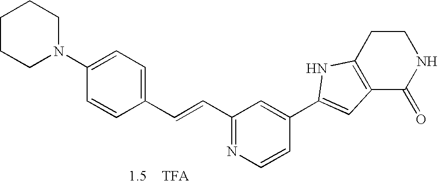 | 2-{2-[(E)-2-(4-pipendin-1-ylphenyl)vinyl]pyridin-4-yl}-1,5,6,7- tetrahydro-4H-pyrroio[3,2-c]pyridin-4-one trifluoroacetate | 0.0558 | |
| 306 | 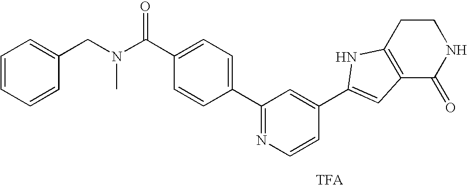 | N-benzyl-N-methyl-4-[4-(4-oxo-4,5,6,7-tetrahydro-1H- pyrrolo[3,2-c]pyridin-2-yl)pyridin-2-yl]benzamide trifluoroacetate | 0.056 | |
| 307 | 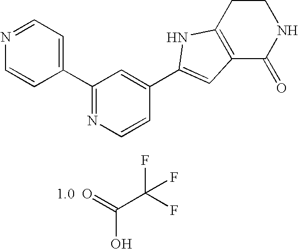 | 2-(2,4′-bipyridin-4-yl)-1,5,6,7-tetrahydro-4H-pyrrolo[3,2-c]pyridin- 4-one trifluoroacetate or V00085961 | 0.0561 | |
| 308 | 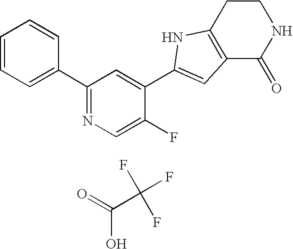 | 2-(5-fluoro-2-phenylpyridin-4-yl)-1,5,6,7-tetrahydro-4H- pyrrolo[3,2-c]pyridin-4-one trifluoroacetate | 0.0565 | |
| 309 | 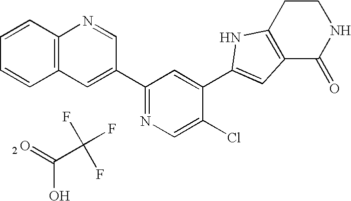 | 2-(5-chloro-2-quinolin-3-ylpyridin-4-yl)-1,5,6,7-tetrahydro-4H- pyrrolo[3,2-c]pyridin-4-one trifluoroacetate | 0.0571 | |
| 310 | 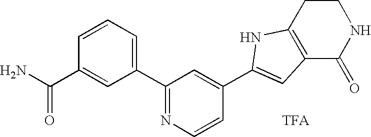 | 3-[4-(4-oxo-4,5,6,7-tetrahydro-1H-pyrrolo[3,2-c]pyridin-2- yl)pyridin-2-yl]benzamide trifluoroacetate | 0.0573 | |
| 311 | 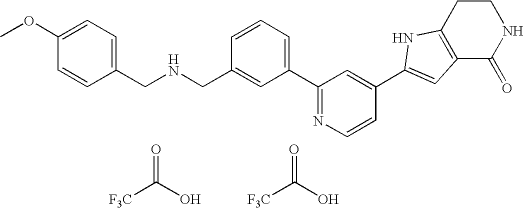 | 2-[2-(3-{[(4-methoxybenzyl)amino]methyl}phenyl)pyridin-4-yl]- 1,5,6,7-tetrahydro-4H-pyrrolo[3,2-c]pyridin-4-one bis(trifluoroacetate) | 0.0573 | |
| 312 | 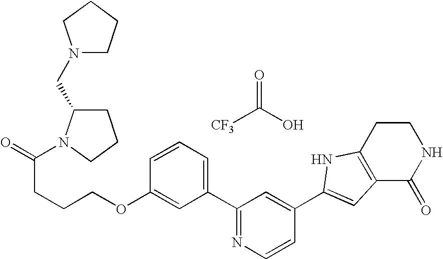 | 2-[2-(3-{4-oxo-4-[(2S)-2-(pyrrolidin-1-ylmethyl)pyrrolidin-1- yl]butoxy}phenyl)pyridin-4-yl]-1,5,6.7-tetrahydro-4H-pyrrolo[3,2- c]pyridin-4-one trifluoroacetate | 0.058 | |
| 313 | 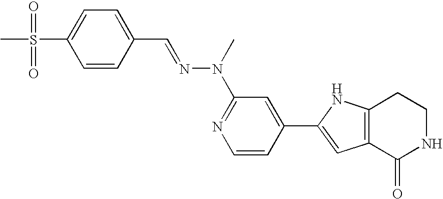 | 4-(methylsulfonyl)benzaldehyde methyl[4-(4-oxo-4,5,6,7- tetrahydro-1H-pyrrolo[3,2-c]pyridin-2-yl)pyridin-2-yl]hydrazone | 0.058 | |
| 314 | 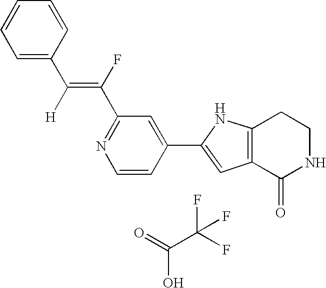 | 2-{2-[(Z)-1-fluoro-2-phenylvinyl]pyridin-4-yl}-1,5,6,7-tetrahydro- 4H-pyrrolo[3,2-c]pyridin-4-one trifluoroacetate | 0.0584 | |
| 315 | 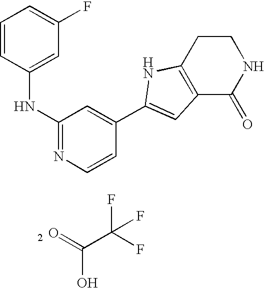 | 2-{2-[(3-fluorophenyl)amino]pyridin-4-yl}-1,5,6,7-tetrahydro-4H- pyrrolo[3,2-c]pyridin-4-one trifluoroacetate | 0.0586 | |
| 316 | 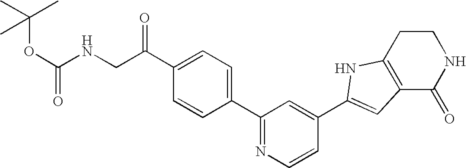 | | 0.0588 | |
| 317 | 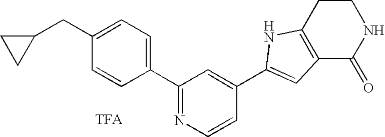 | 2-{2-[4-(cyclopropylmethyl)phenyl]pyridin-4-yl}-1,5,6,7- tetrahydro-4H-pyrrolo[3,2-c]pyridin-4-one trifluoroacetate | 0.0591 | |
| 318 | 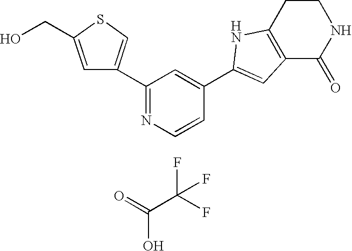 | 2-{2-[5-(hydroxymethyl)thien-3-yl]pyridin-4-yl}-1,5,6,7-tetrahydro- 4H-pyrrolo[3,2-c]pyridin-4-one trifluoroacetate | 0.0593 | |
| 319 | 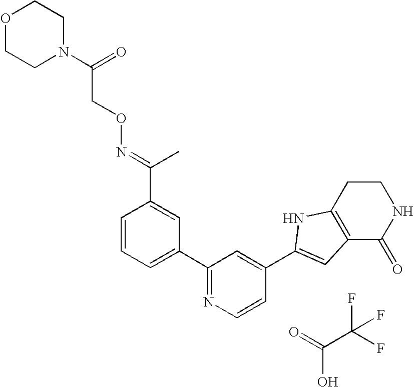 | 2-(2-{3-[(1E)-N-(2-morpholin-4-yl-2- oxoethoxy)ethanimidoyl]phenyl}pyridin-4-yl)-1,5,6,7-tetrahydro- 4H-pyrrolo[3,2-c]pyridin-4-one trifluoroacetate | 0.06 | |
| 320 | 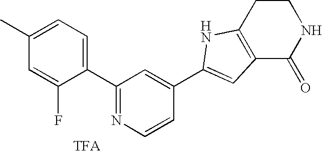 | 2-[2-(2-fluoro-4-methylphenyl)pyridin-4-yl]-1,5,6,7-tetrahydro-4H- pyrrolo[3,2-c]pyridin-4-one trifluoroacetate | 0.0602 | |
| 321 | 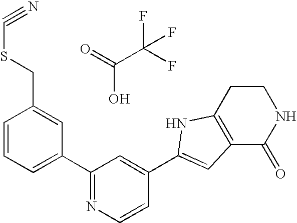 | 3-[4-(4-oxo-4,5,6,7-tetrahydro-1H-pyrrolo[3,2-c]pyridin-2- yl)pyridin-2-yl]benzyl thiocyanate trifluoroacetate | 0.0604 | |
| 322 | 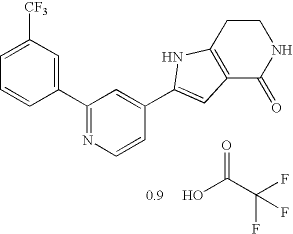 | 2-{2-[3-(trifluoromethyl)phenyl]pyridin-4-yl)-1,5,6,7-tetrahydro- 4H-pyrrolo[3,2-c]pyridin-4-one trifluoroacetate | 0.0606 | |
| 323 | 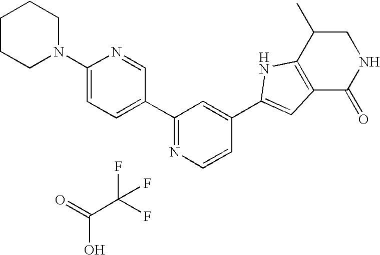 | 7-methyl-2-(6′-piperidin-1-yl-2,3′-bipyridin-4-yl)-1,5,6,7- tetrahydro-4H-pyrrolo[3,2-c]pyridin-4-one trifluoroacetate | 0.0606 | |
| 324 | 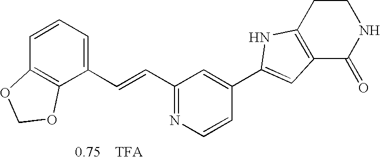 | 2-{2-[(E)-2-(1,3-benzodioxol-4-yl)vinyl]pyridin-4-yl)-1,5,6,7- tetrahydro-4H-pyrrolo[3,2-c]pyridin-4-one trifluoroacetate | 0.0609 | |
| 325 | 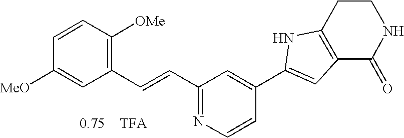 | 2-{2-[(E)-2-(2,5-dimethoxypheny))vinyl]pyridin-4-yl}-1,5,6,7- tetrahydro-4H-pyrrolo[3,2c]pyridin4-one trifluoroacetate | 0.0618 | |
| 326 | 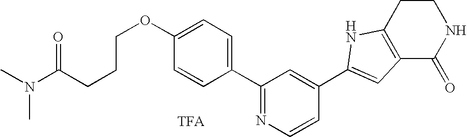 | N,N-dimethyl-4-{4-[4-(4-oxo-4,5,6,7-tetrahydro-1H-pyrrolo[3,2- c]pyridin-2-yl)pyridin-2-yl]phenoxy}butanamide trifluoroacetate | 0.0622 | |
| 327 | 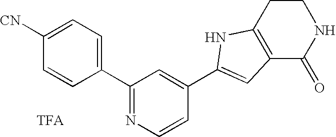 | 4-[4-(4-oxo-4,5,6,7-tetrahydro-1H-pyrrolo[3,2-c]pyridin-2- yl)pyridin-2-yl]benzonitrile trifluoroacetate | 0.0623 | |
| 328 | 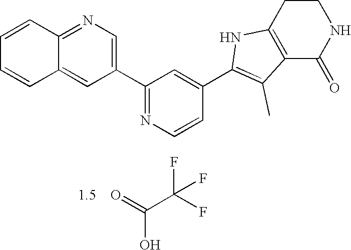 | 3-methyl-2-(2-quinolin-3-ylpyridin-4-yl)-1,5,6,7-tetrahydro-4H- pyrrolo[3,2-c]pyridin-4-one trifluoroacetate | 0.0624 | |
| 329 | 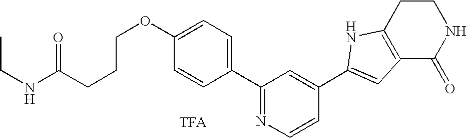 | N-ethyl-4-{4-[4-(4-oxo-4,5,6,7-tetrahydro-1H-pyrrolo[3,2- c]pyridin-2-yl)pyridin-2-yl]phenoxy}butanamide trifluoroacetate | 0.0631 | |
| 330 | 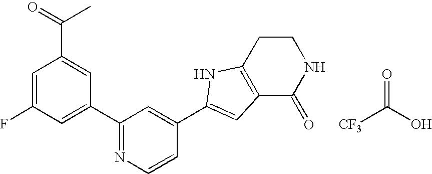 | 2-[2-(3-acetyl-5-fluorophenyl)pyridin-4-yl]-1,5,6,7-tetrahydro-4H- pyrrolo[3,2-c]pyridin-4-one trifluoroacetate | 0.0632 | |
| 331 | 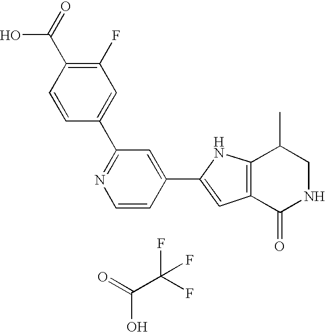 | 2-fluoro-4-[4-(7-methyl-4-oxo-4,5,6,7-tetrahydro-1H-pyrrolo[3,2- c]pyridin-2-yl)pyridin-2-yl]benzoic acid trifluoroacetate | 0.0635 | |
| 332 | 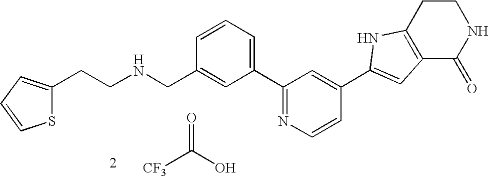 | 2-[2-(3-{[(2-thien-2-ylethyl)amino]methyl}phenyl)pyridin-4-yl]- 1,5,6,7-tetrahydro-4H-pyrrolo[3,2-c]pyridin-4-one trifluoroacetate | 0.0641 | |
| 333 | 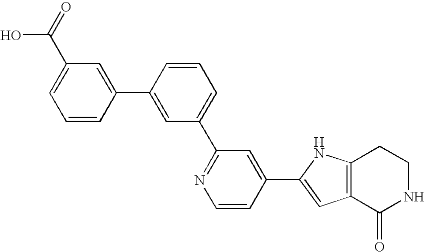 | 3′-[4-(4-axo-4,5,6,7-tetrahydro-1H-pyrrolo[3,2-c]pyridin-2- yl)pyridin-2-yl]-1,1′-biphenyl-3-carboxylic acid | 0.0642 | |
| 334 | 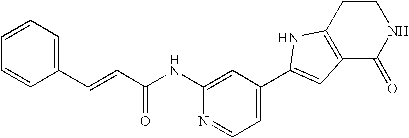 | (2E)-N-[4-(4-oxo-4,5,6,7-tetrahydro-1H-pyrrolo[3,2-c]pyiidin-2- yl)pyridin-2-yl]-3-phenylacrylamide | 0.0643 | |
| 335 | 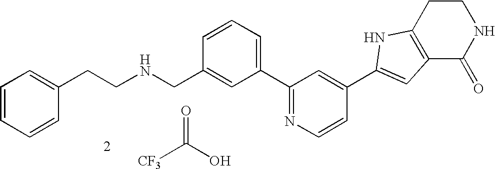 | 2-[2-(3-{[(2-phenylethyl)amino]methyl}phenyl)pyridin-4-yl]- 1,5,6,7-tetrahydro-4H-pyrrolo[3,2-c]pyridin-4-one trifluoroacetate | 0.0644 | |
| 336 | 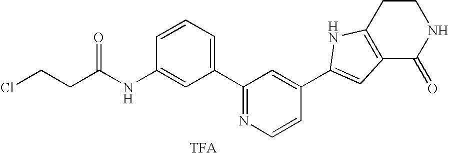 | 3-chloro-N-{3-[4-(4-oxo-4,5,6,7-tetrahydro-1H-pyrrolo[3,2- c]pyridin-2-yl)pyridin-2-yl]phenyl}propanamide trifluoroacetate | 0.0645 | |
| 337 | 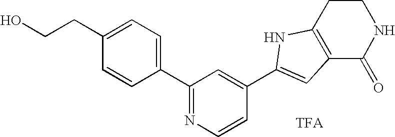 | 2-{2-[4-(2-hydroxyethyl)phenyl]pyridin-4-yl}-1,5,6,7-tetrahydro- 4H-pyrrolo[3,2-c]pyridin-4-one trifluoroacetate | 0.0647 | |
| 338 | 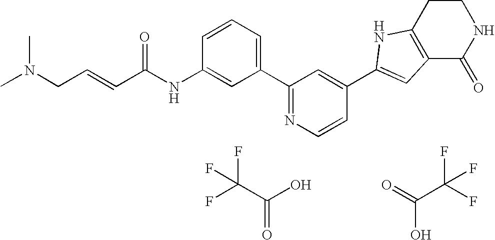 | (2E)-4-(dimethylamino)-N-{3-[4-(4-oxo-4,5,6,7-tetrahydro-1H- pyrrolo[3,2-c]pyridin-2-yl)pyridin-2-yl]phenyl}but-2-enamide bis(trifluoroacetate) | 0.0648 | |
| 339 | 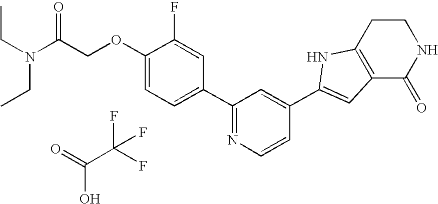 | N,N-diethyl-2-{2-fluoro-4-[4-(4-oxo-4,5,6,7-tetrahydro-1H- pyrrolo[3,2-c]pyridin-2-yl)pyridin-2-yl]phenoxy}acetamide trifluoroacetate | 0.0651 | |
| 340 | 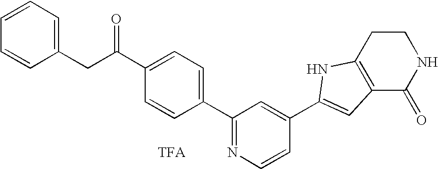 | 2-{2-(4-(phenylacetyl)phenyl]pyridin-4-yl}-1,5,6,7-tetrahydro-4H- pyrrolo[3,2-c]pyridin-4-one trifluoroacetate | 0.0653 | |
| 341 | 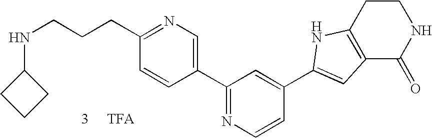 | 2-{6′-[3-(cyclObutylaminO)propyl]-2,3′-bipyridin-4-yl}-1,5,6,7- tetrahydro-4H-pyrrolo[3,2-c]pyridin-4-one trifluoroacetate | 0.0653 | |
| 342 | 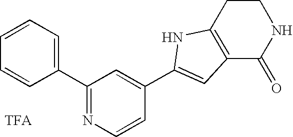 | 2-(2-phenylpyridin-4-yl)-1,5,6,7-tetrahydro-4H-pyrrolo[3,2- c]pyridin-4-one trifluoroacetate | 0.0658 | |
| 343 | 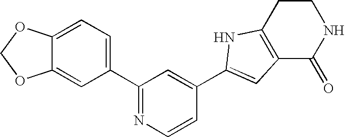 | 2-[2-(1,3-benzodioxol-5-yl)pyridin-4-yl]-1,5,6,7-tetrahydro-4H- pyrrolo[3,2-c]pyridin-4-one | 0.0659 | |
| 344 | 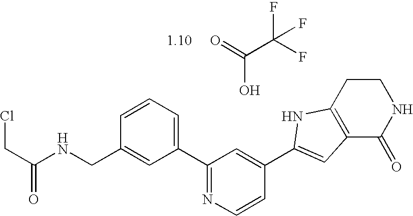 | 2-chloro-N-{3-[4-(4-oxo-4,5,6,7-tetrahydro-1H-pyrrolo[3,2- c]pyridin-2-yl)pyridin-2-yl]benzyl}acetamide trifluoroacetate | 0.0659 | |
| 345 | 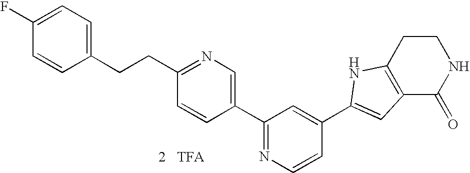 | 2-{6′-[2-(4-fluorophenyl)ethyl]-2,3′-bipyridin-4-yl}-1,5,6,7- tetrahydro-4H-pyrrolo[3,2-c]pyridin-4-one trifluoroacetate | 0.0661 | |
| 346 | 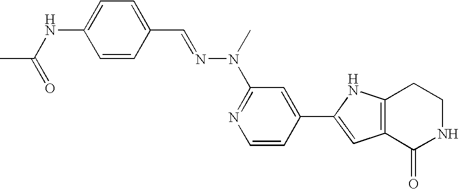 | N-[4-((E)-{2-methyl-2-[4-(4-oxo-4,5,6,7-tetrahydro-1H- pyrrolo[3,2-c]pyridin-2-yl)pyridin-2- yl]hydrazono}methyl)phenyl]acetamide | 0.0662 | |
| 347 | 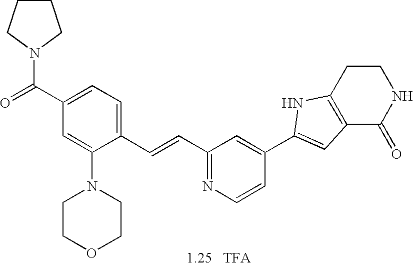 | 2-(2-{(E)-2-[2-morpholin-4-yl-4-(pyrrolidin-1- ylcarbonyl)phenyl]vinyl}pyridin-4-yl)-1,5,6,7-tetrahydro-4H- pyrrolo[3,2-c]pyridin-4-one trifluoroacetate | 0.0663 | |
| 348 | 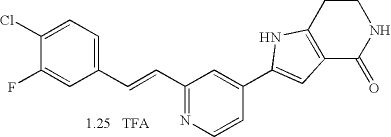 | 2-{2-[(E)-2-(4-chloro-3-fluorophenyl)vinyl]pyridin-4-yl}-1,5,6,7- tetrahydro-4H-pyrrolo[3,2-c]pyridin-4-one trifluoroacetate | 0.0664 | |
| 349 | 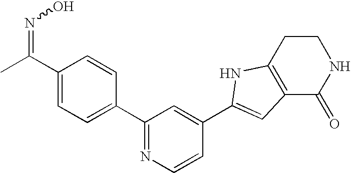 | 2-(2-{4-[(1Z)-N-hydroxyethanimidoyl]phenyl}pyridin-4-yl)-1,5,6,7- tetrahydro-4H-pyrrolo[3,2-c]pyridin-4-one | 0.0667 | |
| 350 | 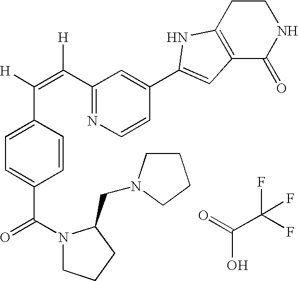 | 2-{2-[(Z)-2-(4-{[(2R)-2-(pyrrolidin-1-ylmethyl)pyrrolidin-1- yl]carbonyl}phenyl)vinyl]pyridin-4-yl}-1,5,6,7-tetrahydro-4H- pyrrolo[3,2-c]pyridin-4-one trifluoroacetate | 0.0667 | |
| 351 | 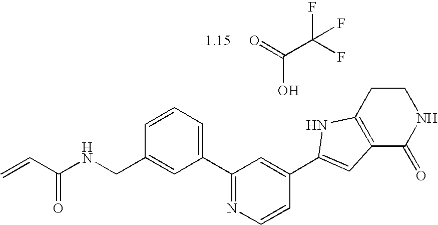 | N-{3-[4-(4-oxo-4,5,6,7-tetrahydro-1H-pyrrolo[3,2-c]pyridin-2- yl)pyridin-2-yl]benzyl}acrylamide trifluoroacetate | 0.0692 | |
| 352 | 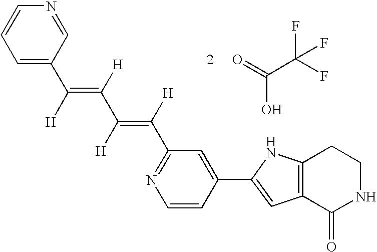 | 2-{2[(1E,3E)-4-pyridin-3-ylbuta-1,3-dienyl]pyridin-4-yl}-1,5,6,7- tetrahydro-4H-pyrrolo[3,2-c]pyridin-4-one trifluoroacetate | 0.0692 | |
| 353 | 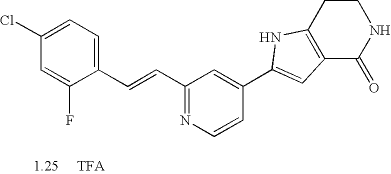 | 2-{2-[(E)-2-(4-chloro-2-fluorophenyl)vinyl]pyridin-4-yl}-1,5,6,7- tetrahydro-4H-pyrrolo[3,2-c]pyridin-4-one trifluoroacetate | 0.0701 | |
| 354 | 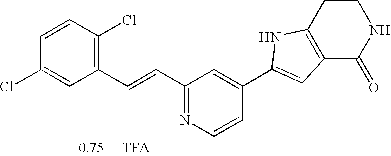 | 2-[2-[(E)-2-(2,5-dichlorophenyl)vinyl]pyridin-4-yl)-1,5,6,7- tetrahydro-4H-pyrrolo[3,2-c]pyridin-4-one trifiuoroacetate | 0.0706 | |
| 355 | 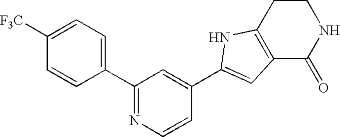 | 2-{2-[4-(trifluoromethyl)phenyl]pyridin-4-yl)-1,5,6,7-tetrahydro- 4H-pyrrolo[3,2-c]pyridin-4-one | 0.0712 | |
| 356 | 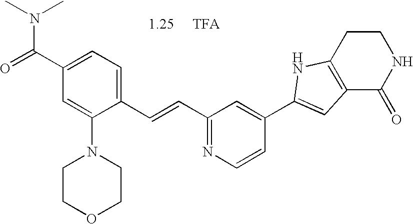 | N,N-dimethyl-3-morpholin-4-yl-4-{(E)-2-[4-(4-oxo-4,5,6,7- tetrahydro-1H-pyrrolo[3,2-c]pyridin-2-yl)pyridin-2- yl]vinyl}benzamide trifluoroacetate | 0.072 | |
| 357 | 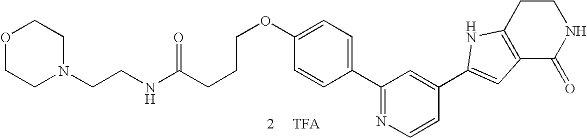 | N-(2-morpholin-4-ylethyl)-4-{4-[4-(4-oxo-4,5,6,7-tetrahydro-1H- pyrrolo[3,2-c]pyridin-2-yl)pyridin-2-yl]phenoxy}butanamide trifluoroacetate | 0.0722 | |
| 358 | 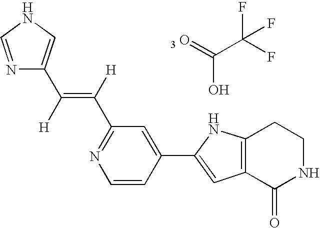 | 2-{2-[(E)-2-(1H-imidazol-4-yl)ethenyl]pyridin-4-yl}-1,5,6,7- tetrahydro-4H-pyrrolo[3,2-clpyridin-4-one trifluoroacetate | 0.0723 | |
| 360 | 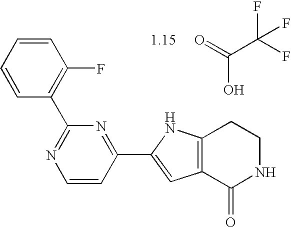 | 2-[2-(2-fluorophenyl)pyrimidin-4-yl]-1,5,6,7-tetrahydro-4H- pyrrolo[3,2-c]pyridin-4-one trifluoroacetate | 0.073 | |
| 360 | 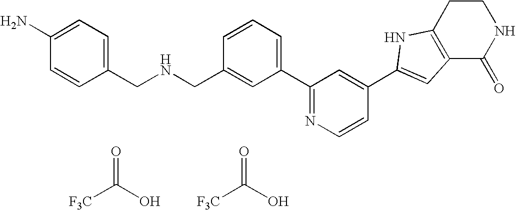 | 2-[2-(3-{[(4-aminobenzyl)amino]methyl)phenyl)pyridin-4-yl]- 1,5,6,7-tetrahydro-4H-pyrrolo[3.2-c]pyridin-4-one bis(trifluoroacetate) | 0.0734 | |
| 361 | 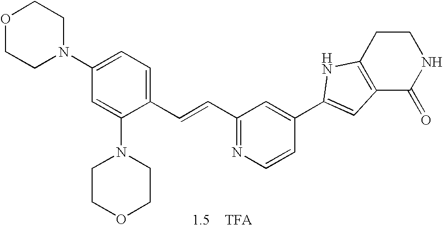 | 2-{2-[(E)-2-(2,4-dimorpholin-4-ylphenyl)vinyl]pyridin-4-yl}- 1,5,6,7-tetrahydro-4H-pyrrolo[3,2-c]pyridin-4-one trifluoroacetate | 0.0737 | |
| 362 | 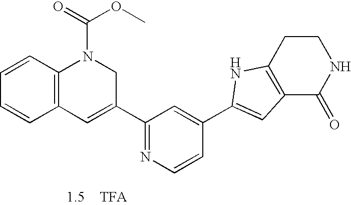 | methyl 3-[4-(4-oxo-4,5,6,7-tetrahydro-1H-pyrrolo[3,2-c]pyridin-2- yl)pyridin-2-yl]quinoline-1(2H)-carboxylate trifluoroacetate | 0.0738 | |
| 363 | 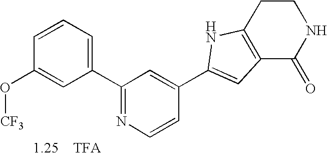 | 2-{2-[3-(trifluoromethoxy)phenyl]pyridin-4-yl{-1,5,6,7-tetrahydro- 4H-pyrrolo[3,2-c]pyridin-4-one trifluoroacetate | 0.075 | |
| 364 | 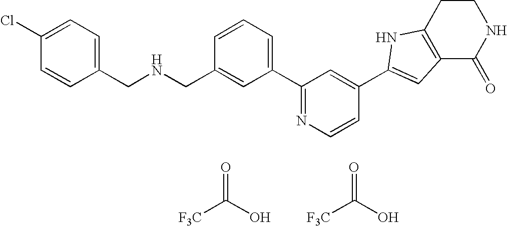 | 2-[2-(3-{[(4-chlorobenzyl)amino]methyl)phenyl)pyridin-4-yl]- 1,5,6,7-tetrahydro-4H-pyrrolo[3,2-c]pyridin-4-one bis(trifluoroacetate) | 0.075 | |
| 365 | 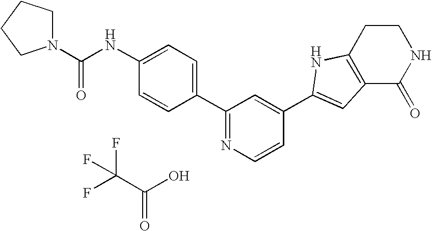 | N-{4-[4-(4-oxo-4,5,6,7-tetrahydro-1H-pyrrolo[3,2-c]pyridin-2- yl)pyridin-2-yl]phenyl}pyrrolidine-1-carboxamide trifluoroacetate | 0.0752 | |
| 366 | 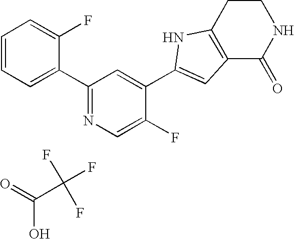 | 2-[5-fluoro-2-(2-fluorophenyl)pyridin-4-yl]-1,5,6,7-tetrahydro-4H- pyrrolo[3,2-c]pyridin-4-one trifluoroacetate | 0.0757 | |
| 367 | 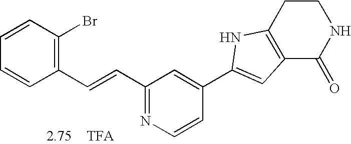 | 2-{2-[(E)-2-(2-bromophenyl)vinyl]pyridin-4-yl}-1,5,6,7-tetrahydro- 4H-pyrrolo(3,2-c]pyridin-4-one trifluoroacetate | 0.0758 | |
| 368 | 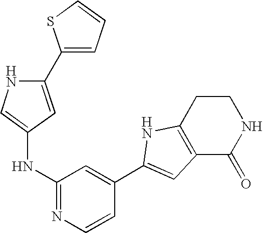 | 2-{2-[(5-thien-2-yl-1H-pyrazol-3-yl)amino]pyridin-4-yl)-1,5,6,7- tetrahydro-4H-pyrrolo[3,2-c]pyridin-4-one | 0.0758 | |
| 369 | 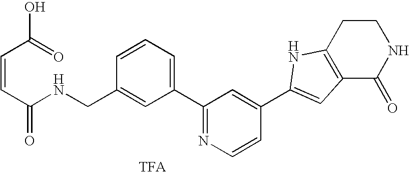 | (2Z)-4-oxo-4-({3-[4-(4-oxo-4,5,6,7-tetrahydro-1H-pyrrolo[3,2- c]pyridin-2-yl)pyridin-2-yl]benzyl)amino)but-2-enoic acid trifluoroacetate | 0.076 | |
| 370 | 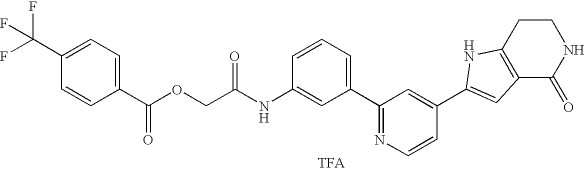 | 2-oxo-2-({3-[4-(4-oxo-4,5,6,7-tetrahydro-1H-pyrrolo[3,2- c]pyridin-2-yl)pyridin-2-yl]phenyl}amino)ethyl 4- (trifluoromethyl)benzoate trifluoroacetate | 0.0766 | |
| 371 | 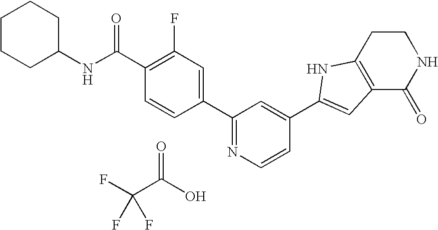 | N-cyclohexyl-2-fluoro-4-[4-(4-oxo-4,5,6,7-tetrahydro-1H- pyrrolo[3,2-c]pyridin-2-y))pyridin-2-yl]benzamide trifluoroacetate | 0.0768 | |
| 372 | 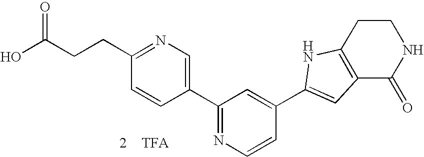 | 3-[4-(4-oxo-4,5,6,7-tetrahydro-1H-pyrrolo[3,2-c]pyridin-2-yl)-2,3′- bipyridin-6′-yl]propanoic acid trifluoroacetate | 0.0775 | |
| 373 | 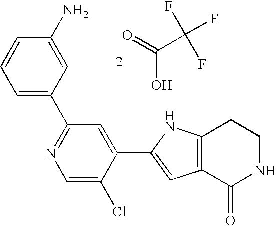 | 2-[2-(3-aminophenyl)-5-chloropyridin-4-yl]-1,5,6,7-tetrahydro- 4H-pyrrolo[3,2-c]pyridin-4-one trifluoroacetate | 0.0777 | |
| 374 | 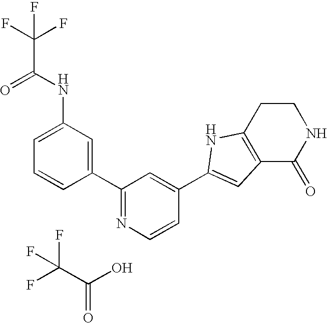 | 2,2,2-trifluoro-N-{3-[4-(4-oxo-4,5,6,7-tetrahydro-1H-pyrrolo[3,2- c]pyridin-2-yl)pyridin-2-yl]phenyl)acetamide trifluoroacetate | 0.0778 | |
| 375 | 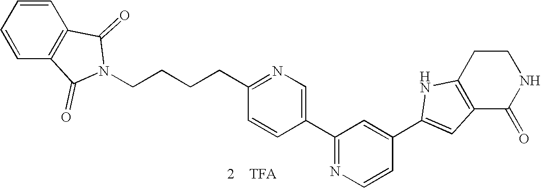 | 2-{4-[4-(4-oxo-4,5,6,7-tetrahydro-1H-pyrrolo[3,2-c]pyridin-2-yl)- 2,3′-bipyridin-6′-yl]butyll-1H-isoindote-1,3(2H)-dione trifluoroacetate | 0.0778 | |
| 376 | 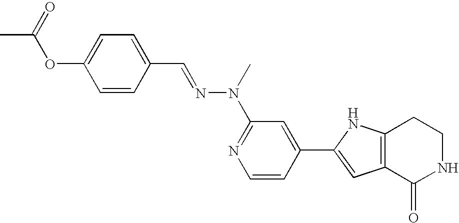 | 4-((E)-{2-methyl-2-[4-(4-oxo-4,5,6,7-tetrahydro-1H-pyrrolo[3,2- c]pyridin-2-yl)pyridin-2-yl]hydrazono}methyl)phenyl acetate | 0.078 | |
| 377 | 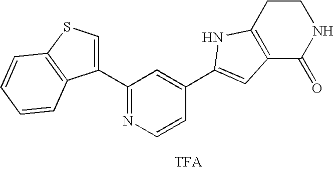 | 2-[2-(1-benzothien-3-yl)pyridin-4-yl]-1,5,6,7-tetrahydro-4H- pyrrolo[3,2-c]pyridin-4-one trifluoroacetate | 0.0781 | |
| 378 | 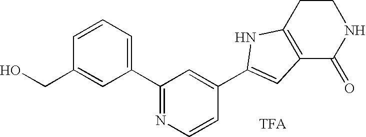 | 2-{2-[3-(hydroxymethyl)phenyl]pyridin-4-yl)-1,5,6,7-tetrahydro- 4H-pyrrolo[3,2-c]pyridin-4-one trifluoroacetate | 0.0797 | |
| 379 | 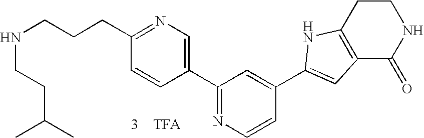 | 2-(6′-{3-[(3-methylbutyl)amino]propyl)-2,3′-bipyridin-4-yl)-1,5,6,7- tetrahydro-4H-pyrrolo[3,2-c]pyridin-4-one trifluoroacetate | 0.0803 | |
| 380 | 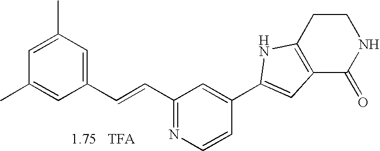 | 2-{2-[(E)-2-(3,5-dimethylphenyl)vinyl]pyridin-4-yl)-1,5,6,7- tetrahydro-4H-pyrrolo[3,2-c]pyridin-4-one trifluoroacetate | 0.0814 | |
| 381 | 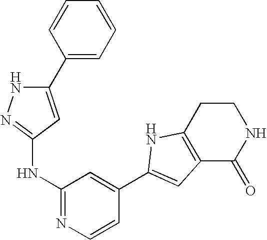 | 2-{2-[(5-phenyl-1H-pyrazol-3-yl)amino]pyridin-4-yl}-1,5,6,7- tetrahydro-4H-pyrrolo[3,2-c]pyridin-4-one | 0.0815 | |
| 382 | 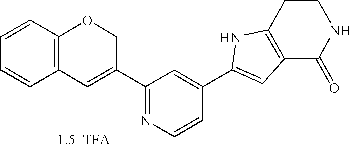 | 2-[2-(2H-chromen-3-yl)pyridin-4-yl]-1,5,6,7-tetrahydro-4H- pyrrolo[3,2-c]pyridin-4-one trifluoroacetate | 0.0817 | |
| 383 | 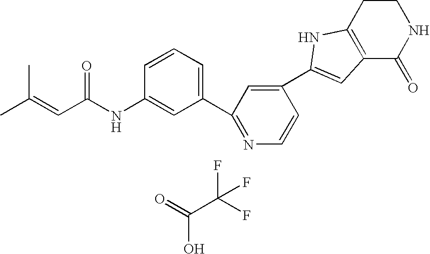 | 3-methyl-N-{3-[4-(4-oxo-4,5,6,7-tetrahydro-1H-pyrrolo[3,2- c]pyridin-2-yl)pyridin-2-yl]phenyl}but-2-enamide trifluoroacetate | 0.0818 | |
| 384 | 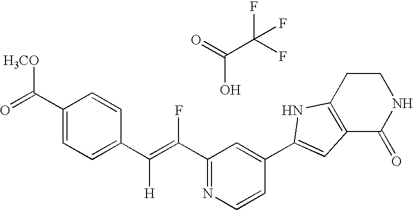 | methyl 4-{(Z)-2-fluoro-2-[4-(4-oxo-4,5,6,7-tetrahydro-1H- pyrrolo[3,2-clpyridin-2-yl)pyridin-2-yl]vinyl}benzoate trifluoroacetate | 0.082 | |
| 385 | 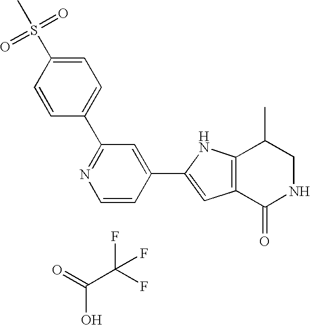 | 7-methyl-2-{2-[4-(methylsulfonyl)phenyl]pyridin-4-yl}-1,5,6,7- tetrahydro-4H-pyrrolo[3,2-c]pyridin-4-one trifluoroacetate | 0.0821 | |
| 386 | 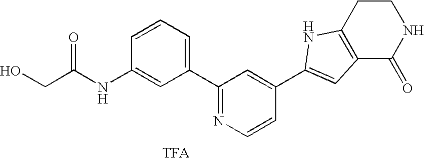 | 2-hydroxy-N-{3-[4-(4-oxo-4,5,6,7-tetrahydro-1H-pyrrolo[3,2- c]pyridin-2-yl)pyridin-2-yl]phenyl}acetamide trifluoroacetate | 0.0823 | |
| 387 | 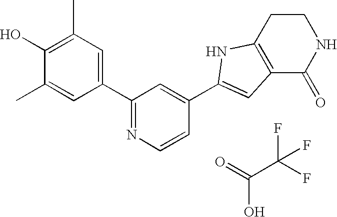 | 2-[2-(4-hydroxy-3,5-dimethylphenyl)pyridin-4-yl]-1,5,6,7- tetrahydro-4H-pyrrolo[3,2-c]pyridin-4-one trifluoroacetate | 0.0827 | |
| 388 | 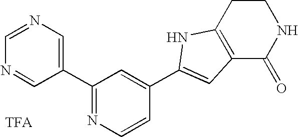 | 2-(2-pyrimidin-5-ylpyridin-4-yl)-1,5,6,7-tetrahydro-4H-pyrrolo[3,2- c]pyridin-4-one trifluoroacetate | 0.0828 | |
| 389 | 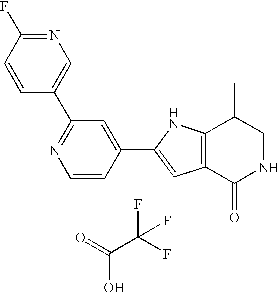 | 2-(6′-fluoro-2,3-bipyridin-4-yl)-7-methyl-1,5,6,7-tetrahydro-4H- pyrrolo[3,2-c]pyridin-4-one trifluoroacetate | 0.0828 | |
| 390 | 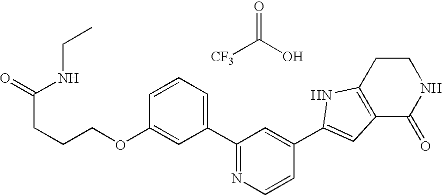 | N-ethyl-4-{3-[4-(4-oxo-4,5,6,7-tetrahydro-1H-pyrrolo[3,2- c]pyridin-2-yl)pyridin-2-yl]phenoxy{butanamide trifluoroacetate | 0.0829 | |
| 391 | 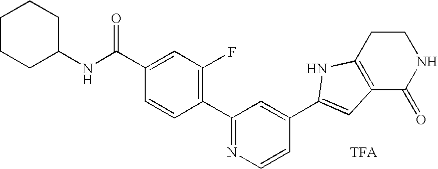 | N-cyclohexyl-3-fluoro-4-[4-(4-oxo-4,5,6,7-tetrahydro-1H- pyrrolo[3,2-c]pyridin-2-yl)pyridin-2-yl]benzamide trifluoroacetate | 0.0831 | |
| 392 | 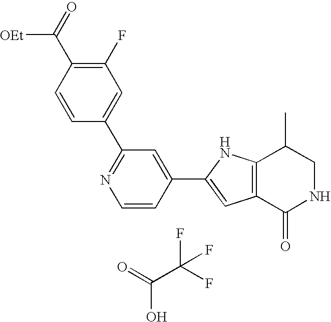 | ethyl 2-fluoro-4-[4-(7-methyl-4-oxo-4,5,6,7-tetrahydro-1H- pyrrolo[3,2-clpyridin-2-yl)pyridin-2-yl]benzoate trifluoroacetate | 0.0831 | |
| 393 | 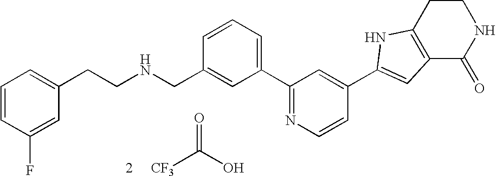 | 2-{2-[3-({[2-(3-fluorophenyl)ethyl]amino}methyl)phenyl]pyridin-4- yl}-1,5,6,7-tetrahydro-4H-pyrrolo[3,2-c]pyridin-4-one trifluoroacetate | 0.0837 | |
| 394 | 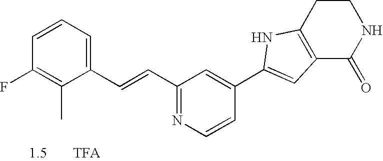 | 2-{2-[(E)-2-(3-fluoro-2-methylphenyl)vinyl]pyridin-4-yl}-1,5,6,7- tetrahydro-4H-pyrrolo[3,2-c]pyridin-4-one trifluoroacetate | 0.0837 | |
| 395 | 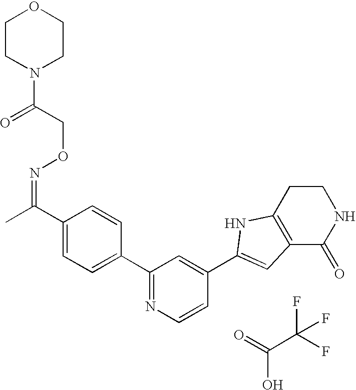 | 2-(2-{4-[(1Z)-N-(2-morpholin-4-yl-2- oxoethoxy)ethanimidoyl]phenyl}pyridin-4-yl)-1,5,6,7-tetrahydro- 4H-pyrrolo[3,2-c]pyridin-4-one trifluoroacetate | 0.0847 | |
| 396 | 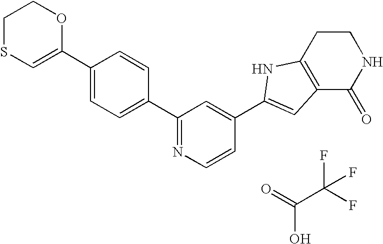 | 2-{2-[4-(5,6-dihydro-1,4-oxathiin-2-yl)phenyl]pyridin-4-yl}-1,5,6,7- tetrahydro-4H-pyrrolo[3,2-c]pyridin-4-one trifluoroacetate | 0.0848 | |
| 397 | 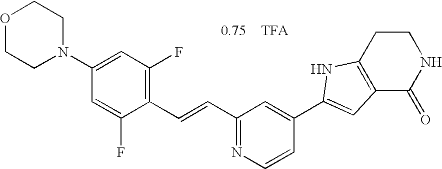 | 2-{2-[(E)-2-(2,6-difluoro-4-morpholin-4-ylphenyl)vinyl]pyridin-4- yl}-1,5,6,7-tetrahydro-4H-pyrrolo[3,2-c]pyridin-4-one trifluoroacetate | 0.085 | |
| 398 | 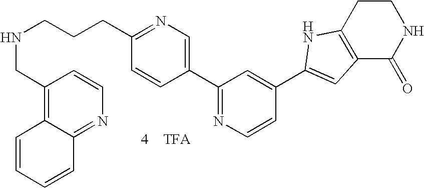 | 2-(6′-(3-[(quinolin-4-ylmethyl)amino]propyfl-2,3′-bipyridin-4-yl)- 1,5,6,7-tetrahydro-4H-pyrrolo[3,2-c]pyridin-4-one trifluoroacetate | 0.0852 | |
| 399 | 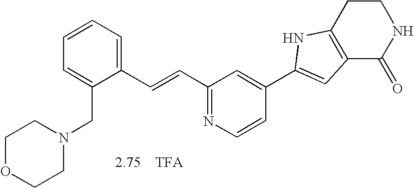 | 2-(2-{(E)-2-[2-(morpholin-4-ylmethyl)phenyl)phenyl]vinyl}pyridin-4-yl)- 1,5,6,7-tetrahydro-4H-pyrrolo[3,2-c]pyridin-4-one trifluoroacetate | 0.0859 | |
| 400 | 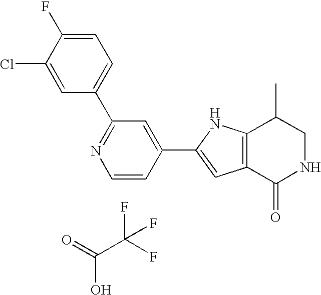 | 2-[2-(3-chloro-4-fluorophenyl)pyridin-4-yl]-7-methyl-1,5,6,7- tetrahydro-4H-pyrrolo[3,2-c]pyridin-4-one trifluoroacetate | 0.0864 | |
| 401 | 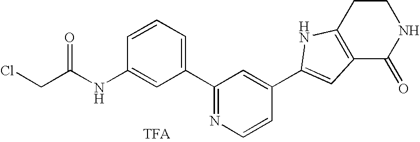 | 2-chloro-N-{3-[4-(4-oxo-4,5,6,7-tetrahydro-1H-pyrrolo[3,2- c]pyridin-2-yl)pyridin-2-yl]phenyl}acetamide trifluoroacetate | 0.0875 | |
| 402 | 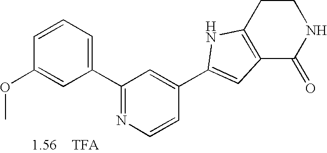 | 2-[2-(3-methoxyphenyl)pyridin-4-yl]-1,5,6,7-tetrahydro-4H- pyrrolo[3,2-c]pyridin-4-one trifluoroacetate | 0.0877 | |
| 403 | 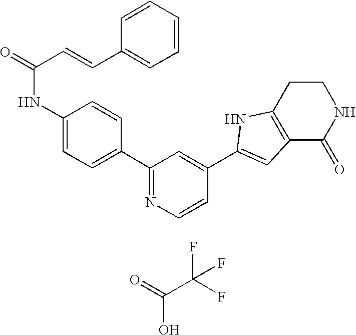 | (2E)-N-{4-[4-(4-oxo-4,5,6,7-tetrahydro-1H-pyrrolo[3,2-c]pyridin- 2-yl)pyridin-2-yl]phenyl}-3-phenylacrylamide trifluoroacetate | 0.0877 | |
| 404 | 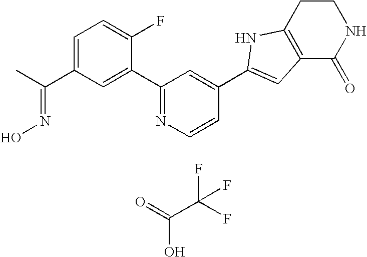 | 2-(2-{2-fluoro-5-[(1E)-N-hydroxyethanimidoyl]phenyl}pyridin-4- yl)-1,5,6,7-tetrahydro-4H-pyrrolo[3,2-c]pyridin-4-one trifluoroacetate | 0.0886 | |
| 405 | 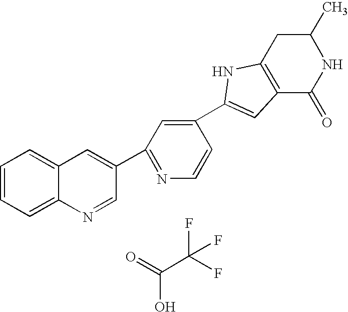 | 6-methyl-2-(2-quinolin-3-ylpyridin-4-yl)-1,5,6,7-tetrahydro-4H- pyrrolo[3,2-c]pyridin-4-one trifluoroacetate | 0.0887 | |
| 406 | 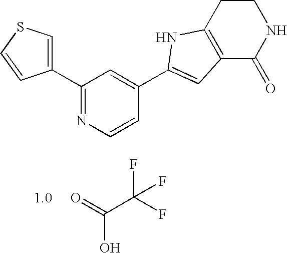 | 2-(2-thien-3-ylpyridin-4-yl)-1,5,6,7-tetrahydro-4H-pyrrolo[3,2- c]pyridin-4-one trifluoroacetate | 0.0891 | |
| 407 | 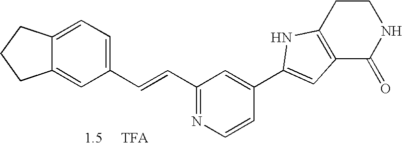 | 2-{2-[(E)-2-(2,3-dihydro-1H-inden-5-yl)vinyl]pyridin-4-yl}-1,5,6,7- tetrahydro-4H-pyrrolo[3,2-c]pyridin-4-one trifluoroacetate | 0.0896 | |
| 408 | 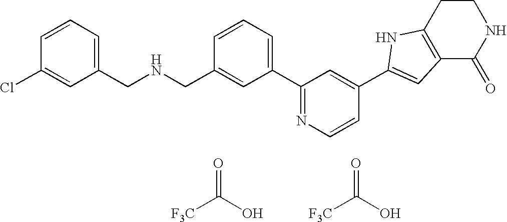 | 2-[2-(3-{[(3-chlorobenzyl)amino]methyl]phenyl)pyridin-4-yl]- 1,5,6,7-tetrahydro-4H-pyrrolo[3,2-c]pyridin-4-one bis(trifluoroacetate) | 0.0899 | |
| 409 | 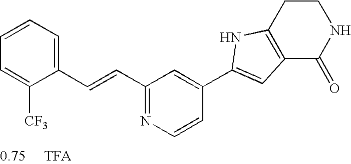 | 2-(2-{(E)-2-[2-(trifluoromethyl)phenyl]vinyl}pyridin-4-yl)-1,5,6,7- tetrahydro-4H-pyrrolo[3,2-c]pyridin-4-one trifluoroacetate | 0.0899 | |
| 410 | 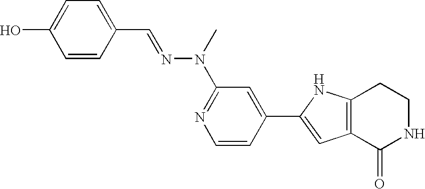 | 4-hydroxybenzaldehyde methyl[4-(4-oxo-4,5,6,7-tetrahydro-1H- pyrrolo[3,2-c]pyridin-2-yl)pyridin-2-yl]hydrazone | 0.0906 | |
| 411 | 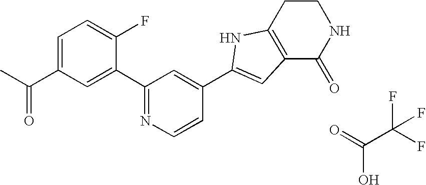 | 2-[2-(5-acetyl-2-fluorophenyl)pyridin-4-yl]-1,5,6,7-tetrahydro-4H- pyrrolo[3,2-c]pyridin-4-one trifluoroacetate | 0.0907 | |
| 412 | 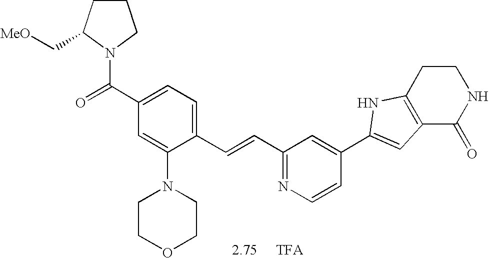 | 2-{2-[(E)-2-(4-{[(2S)-2-(methoxymethyl)pyrrolidin-1-yl]carbonyl}- 2-morpholin-4-ylphenyl)vinyl]pyridin-4-yl}-1,5,6,7-tetrahydro-4H- pyrrolo[3,2-c]pyridin-4-one trifluoroacetate | 0.0908 | |
| 413 | 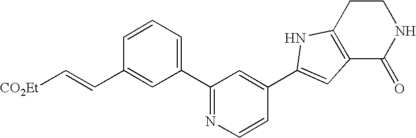 | ethyl (2E)-3-{3-[4-(4-oxo-4,5,6,7-tetrahydro-1H-pyrrolo[3,2- c]pyridin-2-yl)pyridin-2-yl]phenyl}acrylate | 0.0909 | |
| 414 | 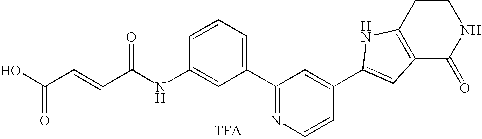 | (2E)-4-oxo-4-({3-[4-(4-oxo-4,5,6,7-tetrahydro-1H-pyrrolo[3,2- c]pyridin-2-yl)pyridin-2-yl]phenyl}amino)but-2-enoic acid trifluoroacetate | 0.0912 | |
| 415 | 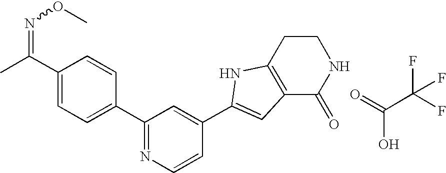 | 2-(2-{4-[(1Z)-N-methoxyethanimidoyl]phenyl}pyridin-4-yl)- 1,5,6,7-tetrahydro-4H-pyrrolo[3,2-c]pyridin-4-one trifluoroacetate | 0.0923 | |
| 416 | 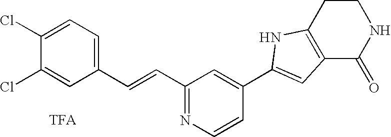 | 2-{2-[(E)-2-(3,4-dichlorophenyl)vinyl]pyridin-4-yl}-1,5,6,7- tetrahydro-4H-pyrrolo[3,2-c]pyridin-4-one trifluoroacetate | 0.0923 | |
| 417 | 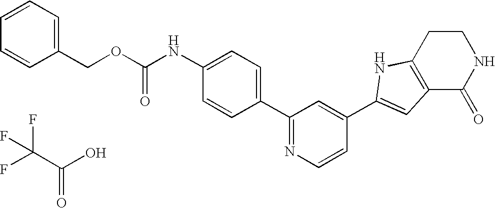 | benzyl 4-[4-(4-oxo-4,5,6,7-tetrahydro-1H-pyrrolo[3,2-c]pyridin-2- yl)pyridin-2-yl]phenylcarbamate trifluoroacetate | 0.0926 | |
| 418 | 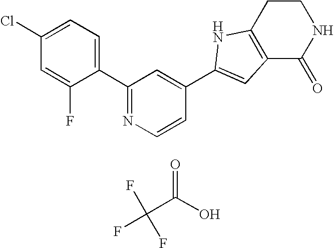 | 2-[2-(4-chloro-2-fluorophenyl)pyridin-4-yl]-1,5,6,7-tetrahydro-4H- pyrrolo[3,2-c]pyridin-4-one trifluoroacetate | 0.093 | |
| 419 | 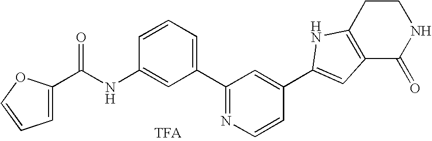 | N-{3-[4-(4-oxo-4,5,6,7-tetrahydro-1H-pyrrolo[3,2-c]pyridin-2- yl)pyridin-2-yl]phenyl}-2-furamide trifluoroacetate | 0.0935 | |
| 420 | 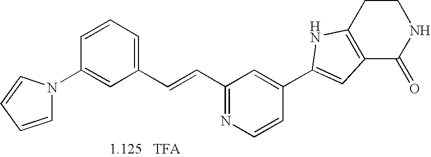 | 2-(2-{(E)-2-[3-(1H-pyrrol-1-yl)phenyl]vinyl}pyridin-4-yl)-1,5,6,7- tetrahydro-4H-pyrrolo[3,2-c]pyridin-4-one trifluoroacetate | 0.0937 | |
| 421 | 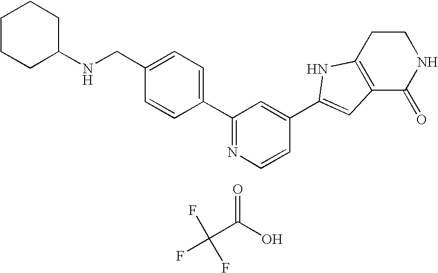 | 2-(2-{4-[(cyclohexylamino)methyl]phenyl}pyridin-4-yl)-1,5,6,7- tetrahydro-4H-pyrrolo[3,2-c]pyridin-4-one trifluoroacetate | 0.0939 | |
| 422 | 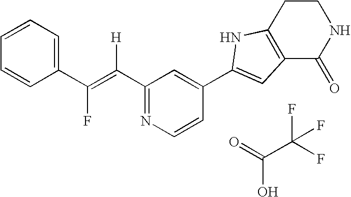 | 2-{2-[(Z)-2-fluoro-2-phenylvinyl]pyridin-4-yl}-1,5,6,7-tetrahydro- 4H-pyrrolo[3,2-c]pyridin-4-one trifluoroacetate | 0.0939 | |
| 423 | 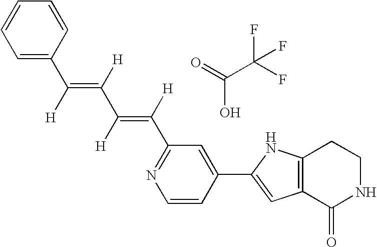 | 2-{2-[(1E,3E)-4-phenylbuta-1,3-dienyl]pyridin-4-yl)-1,5,6,7- tetrahydro-4H-pyrrolo[3,2-c]pyridin-4-one trifluoroacetate | 0.0953 | |
| 424 | 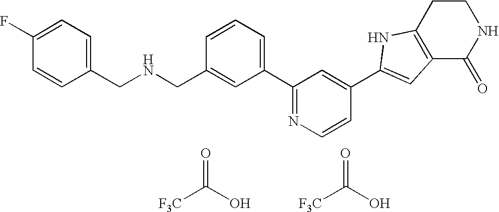 | 2-[2-(3-{[(4-fluorobenzyl)amino]methyl)phenyl}pyridin-4-yl]- 1,5,6,7-tetrahydro-4H-pyrrolo[3,2-c]pyridin-4-one bis(trifluoroacetate) | 0.097 | |
| 425 | 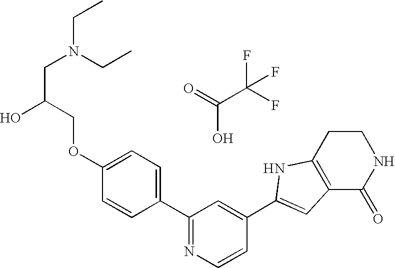 | 2-(2-{4-[3-(diethylamino)-2-hydroxypropoxy]phenyl}pyridin-4-yl)- 1,5,6,7-tetrahydro-4H-pyrrolo[3,2-c]pyridin-4-one trifluoroacetate | 0.0972 | |
| 426 | 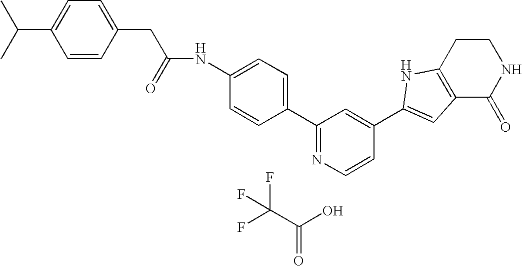 | 2-(4-isopropylphenyl)-N-{4-[4-(4-oxo-4,5,6,7-tetrahydro-1H- pyrrolo[3,2-c]pyridin-2-yl)pyridin-2-yl]phenyl}acetamide trifluoroacetate | 0.0975 | |
| 427 | 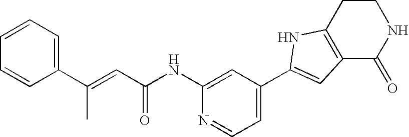 | (2E)-N-[4-(4-oxo-4,5,6,7-tetrahydro-1H-pyrrolo[3,2-c]pyridin-2- yl)pyridin-2-yl]-3-phenylbut-2-enamide | 0.098 | |
| 428 | 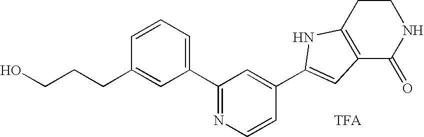 | 2-{2-[3-(3-hydroxypropyl)phenyl]pyridin-4-yl}-1,5,6,7-tetrahydro- 4H-pyrrolo[3,2-c]pyridin-4-one trifluoroacetate | 0.0983 | |
| 429 | 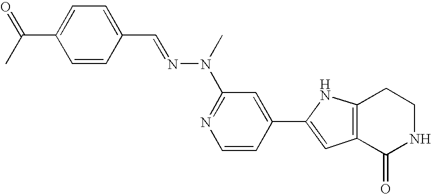 | 4-acetylbenzaldehyde methyl[4-(4-oxo-4,5,6,7-tetrahydro-1H- pyrrolo[3,2-c]pyridin-2-yl)pyridin-2-yl]hydrazone | 0.0987 | |
| 430 | 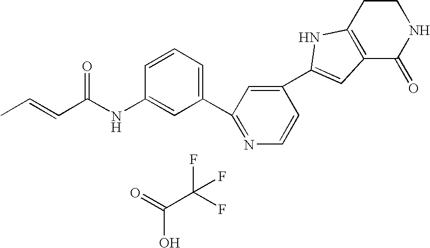 | (2E)-N-{3-[4-(4-oxo-4,5,6,7-tetrahydro-1H-pyrrolo[3,2-c]pyridin- 2-yl)pyridin-2-yl]phenyl}but-2-enamide trifluoroacetate | 0.0993 | |
| 431 | 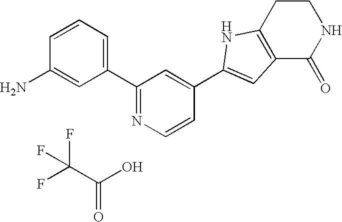 | 2-[2-(3-aminophenyl)pyridin-4-yl]-1,5,6,7-tetrahydro-4H- pyrrolo[3,2-c]pyridin-4-one trifluoroacetate | 0.0993 | |
| 432 | 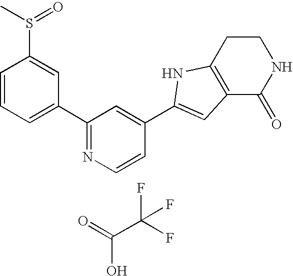 | 2-{2-[3-(methylsulfinyl)phenyl]pyridin-4-yl}-1,5,6,7-tetrahydro-4H- pyrrolo[3,2-c]pyridin-4-one trifluoroacetate | 0.0996 | |
| 433 | 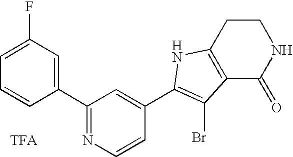 | 3-bromo-2-[2-(3-fluorophenyl)pyridin-4-yl]-1,5,6,7-tetrahydro-4H- pyrrolo[3,2-c]pyridin-4-one trifluoroacetate | 0.1 | |
| 434 | 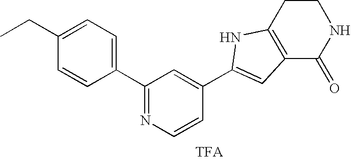 | 2-[2-(4-ethylphenyl)pyridin-4-yl]-1,5,6,7-tetrahydro-4H- pyrrolo[3,2-c]pyridin-4-one trifluoroacetate | 0.101 | |
| 435 | 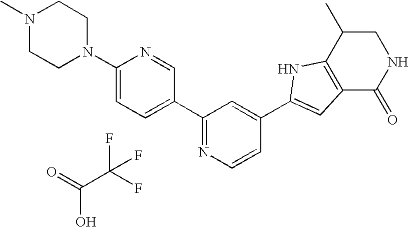 | 7-methyl-2-[6′-(4-methylpiperazin-1-yl)-2,3-bipyridin-4-yl]- 1,5,6,7-tetrahydro-4H-pyrralo[3,2-c]pyridin-4-one trifluoroacetate | 0.101 | |
| 436 | 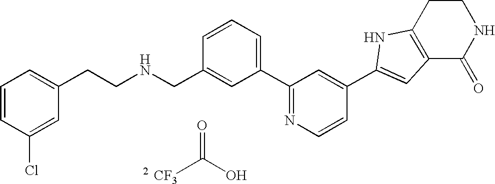 | 2-{2-[3-({[2-(3-chlorophenyl)ethyl]amino}methyl)phenyl]pyridin-4- yl}-1,5,6,7-tetrahydro-4H-pyrrolo[3,2-c]pyridin-4-one trifluoroacetate | 0.101 | |
| 437 | 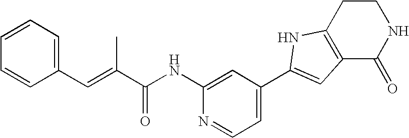 | (2E)-2-methyl-N-[4-(4-oxo-4,5,6,7-tetrahydro-1H-pyrrolo[3,2- c]pyridin-2-yl)pyridin-2-yl]-3-phenylacrylamide | 0.102 | |
| 438 | 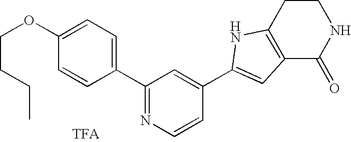 | 2-[2-(4-butoxyphenyl)pyridin-4-yl]-1,5,6,7-tetrahydro-4H- pyrrolo[3,2-c]pyridin-4-one trifluoroacetate | 0.104 | |
| 439 | 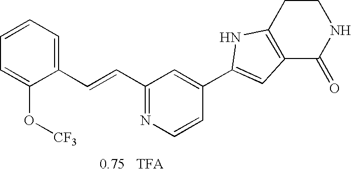 | 2-(2-{(E)-2-[2-(trifluoromethoxy)phenyl]vinyl}pyridin-4-yl)-1,5,6,7- tetrahydro-4H-pyrrolo[3,2-c]pyridin-4-one trifluoroacetate | 0.104 | |
| 440 | 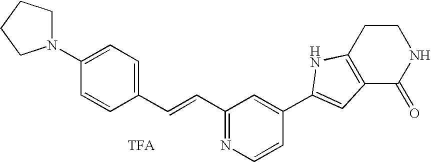 | 2-{2-[(E)-2-(4-pyrrolidin-1-ylphenyl)vinyl]pyridin-4-yl}-1,5,6,7- tetrahydro-4H-pyrrolo[3,2-c]pyridin-4-one trifluoroacetate | 0.106 | |
| 441 | 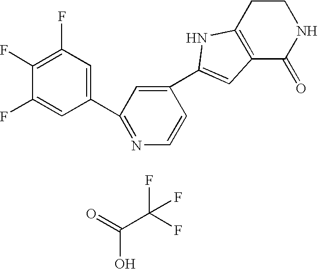 | 2-[2-(3,4,5-trifluorophenyl)pyridin-4-yl]-1,5,6,7-tetrahydro-4H- pyrrolo[3,2-c]pyridin-4-one trifluoroacetate | 0.108 | |
| 442 | 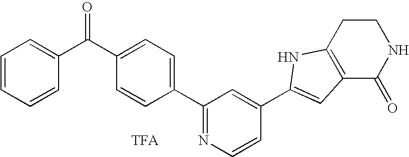 | 2-[2-(4-benzoylphenyl)pyridin-4-yl]-1,5,6,7-tetrahydro-4H- pyrrolo[3,2-c]pyridin-4-one trifluoroacetate | 0.109 | |
| 443 | 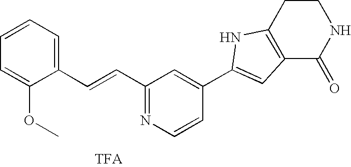 | 2-{2-[(E)-2-(2-methoxyphenyl)vinyl]pyridin-4-yl}-1,5,6,7- tetrahydro-4H-pyrrolo[3,2-c]pyridin-4-one trifluoroacetate | 0.109 | |
| 444 | 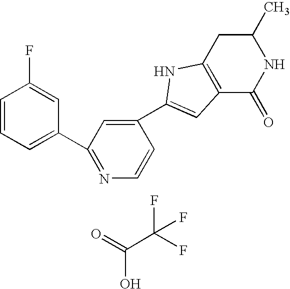 | 2-[2-(3-fluorophenyl)pyridin-4-yl]-6-methyl-1,5,6,7-tetrahydro-4H- pyrrolo[3,2-c]pyridin-4-one trifluoroacetate | 0.11 | |
| 445 | 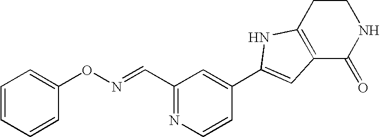 | 4-(4-oxo-4,5,6,7-tetrahydro-1H-pyrrolo[3,2-c]pyridin-2- yl)pyridine-2-carbaldehyde O-phenyloxime | 0.11 | |
| 446 | 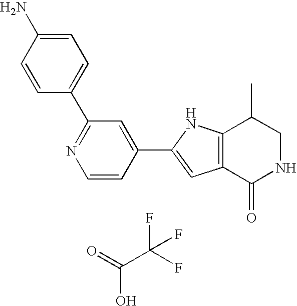 | 2-[2-(4-aminophenyl)pyridin-4-yl]-7-methyl-1,5,6,7-tetrahydro- 4H-pyrrolo[3,2-c]pyridin-4-one trifluoroacetate | 0.111 | |
| 447 | 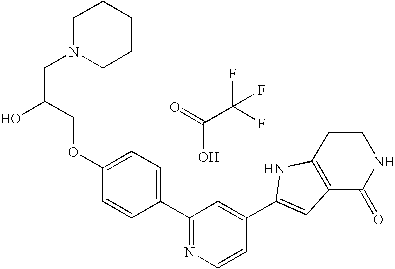 | 2-{2-(4-(2-hydroxy-3-piperidin-1-ylpropoxy)phenyl]pyridin-4-yl}- 1,5,6,7-tetrahydro-4H-pyrrolo[3,2-c]pyridin-4-one trifluoroacetate | 0.112 | |
| 448 | 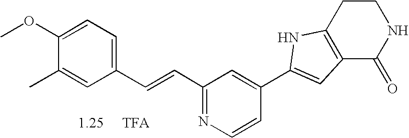 | 2-{2-[(E)-2-(4-methoxy-3-methylphenyl)vinyl]pyridin-4-yl}- 1,5,6,7-tetrahydro-4H-pyrrolo[3,2-c]pyridin-4-one trifluoroacetate | 0.112 | |
| 449 | 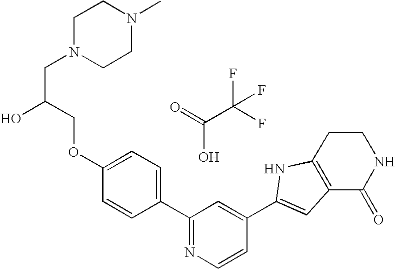 | 2-(2-{4-[2-hydroxy-3-(4-methylpiperazin-1- yl)propoxy]phenyl}pyridin-4-yl)-1,5,6,7-tetrahydro-4H-pyrrolo[3,2 c]pyridin-4-one trifluoroacetate | 0.113 | |
| 450 | 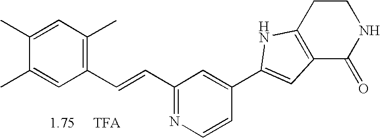 | 2-{2-[(E)-2-(2,4,5-trimethylphenyl)vinyl]pyridin-4-yl}-1,5,6,7- tetrahydro-4H-pyrrolo[3,2-c]pyridin-4-one trifluoroacetate | 0.113 | |
| 451 | 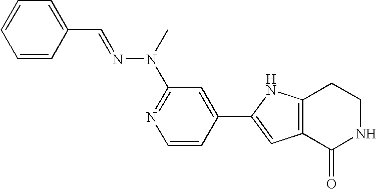 | benzaldehyde methyl[4-(4-oxo-4,5,6,7-tetrahydro-1H- pyrrolo[3,2-c]pyridin-2-yl)pyridin-2-yl]hydrazone | 0.113 | |
| 452 | 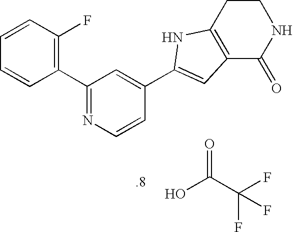 | 2-[2-(2-fluorophenyl)pyridin-4-yl]-1,5,6,7-tetrahydro-4H- pyrrolo[3,2-c]pyridin-4-one trifluoroacetate | 0.114 | |
| 453 | 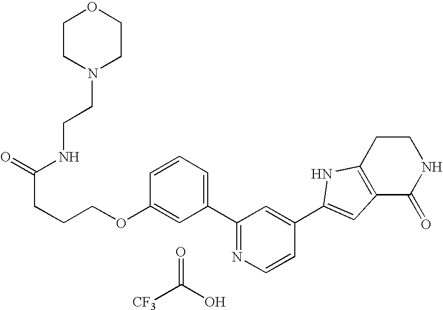 | N-(2-morpholin-4-ylethyl)-4-{3-[4-(4-oxo-4,5,6,7-tetrahydro-1H- pyrrolo[3,2-c]pyridin-2-yl)pyridin-2-yl]phenoxy}butanamide trifluoroacetate | 0.114 | |
| 454 | 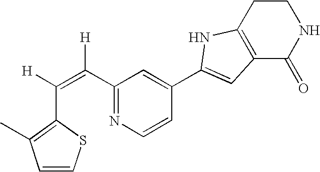 | 2-{2-[(Z)-2-(3-methylthien-2-yl)vinyl]pyridin-4-yl}-1,5,6,7- tetrahydro-4H-pyrrolo[3,2-c]pyridin-4-one | 0.114 | |
| 455 | 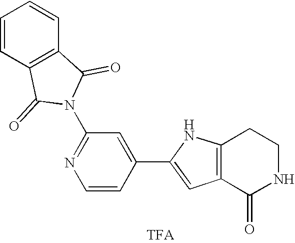 | 2-[4-(4-oxo-4,5,6,7-tetrahydro-1H-pyrrolo[3,2-c]pyridin-2- yl)pyridin-2-yl]-1H-isoindole-1,3(2H)-dione trifluoroacetate | 0.114 | |
| 456 | 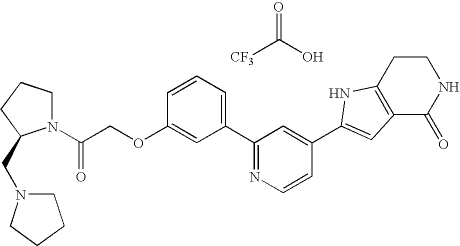 | 2-[2-(3-{2-oxo-2-[(2R)-2-(pyrrolidin-1-ylmethyl)pyrrolidin-1- yl]ethoxylphenyl)pyridin-4-yl-1,5,6,7-tetrahydro-4H-pyrrolo[3,2- c]pyridin-4-one trifluoroacetate | 0.115 | |
| 457 | 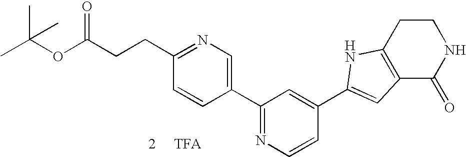 | tert-butyl 3-[4-(4-oxo-4,5,6,7-tetrahydro-1H-pyrrolo[3,2-c]pyridin 2-yl)-2,3′-bipyridin-6′-yl]propanoate trifluoroacetate | 0.115 | |
| 458 | 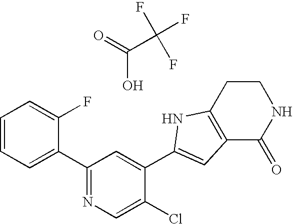 | 2-[5-chloro-2-(2-fluorophenyl)pyridin-4-yl]-1,5,6,7-tetrahydro-4H- pyrrolo[3,2-c]pyridin-4-one trifluoroacetate | 0.115 | |
| 459 | 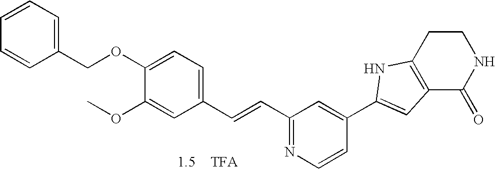 | 2-(2-{(E)-2-f4-(benzyloxy)-3-methoxyphenyl]vinyl}pyridin-4-yl)- 1,5,6,7-tetrahydro-4H-pyrrolo[3,2-c]pyridin-4-one trifluoroacetate | 0.115 | |
| 460 | 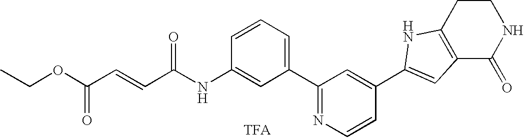 | ethyl (2E)-4-oxo-4-({3-[4-(4-oxo-4,5,6,7-tetrahydro-1H- pyrrolo[3,2-c]pyridin-2-yl)pyridin-2-yl]phenyl}amino)but-2-enoate trifluaroacetate | 0.116 | |
| 461 | 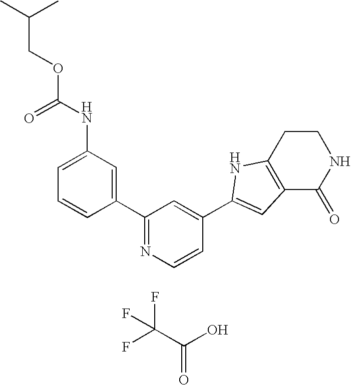 | isobutyl 3-[4-(4-oxo-4,5,6,7-tetrahydro-1H-pyrrolo[3,2-c]pyridin- 2-yl)pyridin-2-yl]phenylcarbamate trifluoroacetate | 0.116 | |
| 462 | 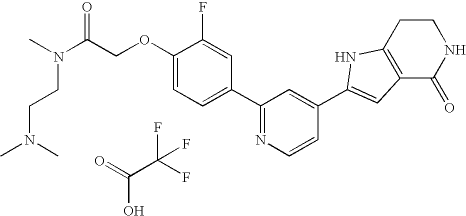 | N-[2-(dimethylamino)ethyl]-2-{2-fluoro-4-[4-(4-oxo-4,5,6,7- tetrahydro-1H-pyrrolo[3,2-c]pyridin-2-yl)pyridin-2-yl]phenoxy}-N methylacetamide trifluoroacetate | 0.116 | |
| 463 | 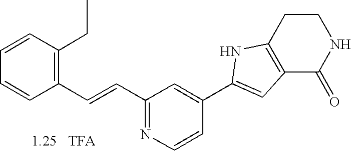 | 2-{2-[(E)-2-(2-ethylphenyl)vinyl]pyridin-4-yll-1,5,6,7-tetrahydro- 4H-pyrrolo[3,2-c]pyridin-4-one trifluoroacetate | 0.116 | |
| 464 | 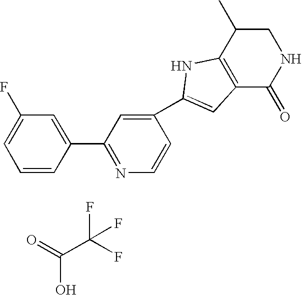 | 2-[2-(3-fluorophenyl)pyridin-4-yl]-7-methyl-1,5,6,7-tetrahydro-4H- pyrrolo[3,2-c]pyridin-4-one trifluoroacetate | 0.117 | |
| 465 | 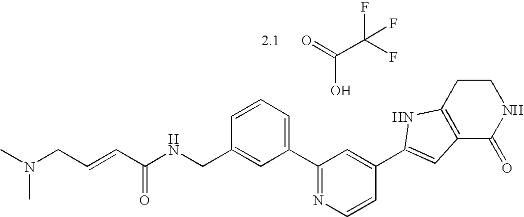 | (2E)-4-(dimethylamino)-N-{3-[4-(4-oxo-4,5,6,7-tetrahydro-1H- pyrrolo[3,2-c]pyridin-2-yl)pyridin-2-yl]benzyl}but-2-enamide trifluoroacetate | 0.118 | |
| 466 | 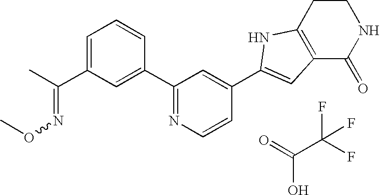 | 2-(2-{3-[(1E)-N-methoxyethanimidoyl]phenyl)pyridin-4-yl)- 1,5,6,7-tetrahydro-4H-pyrrolo[3,2-c]pyridin-4-one trifluoroacetate | 0.118 | |
| 467 | 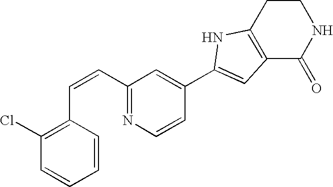 | 2-{2-[(Z)-2-(2-chlorophenyl)vinyl]pyridin-4-yl}-1,5,6,7-tetrahydro- 4H-pyrrolo[3,2-c]pyridin-4-one | 0.118 | |
| 468 | 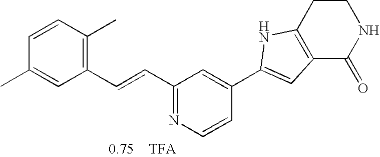 | 2-{2-[(E)-2-(2,5-dimethylphenyl)vinyl]pyridin-4-yl}-1,5,6,7- tetrahydro-4H-pyrrolo[3,2-c]pyridin-4-one trifluoroacetate | 0.118 | |
| 469 | 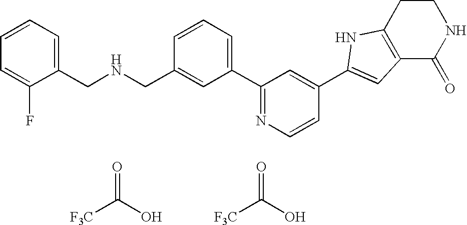 | 2-[2-(3-{[(2-fluorobenzyl)amino]methyl}phenyl)pyridin-4-yl]- 1,5,6,7-tetrahydro-4H-pyrrolo[3,2-c]pyridin-4-one bis(trifluoroacetate) | 0.119 | |
| 470 | 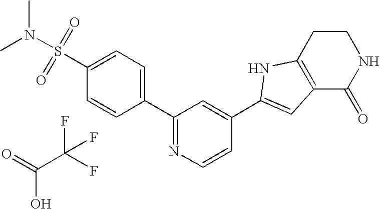 | N,N-dimethyl-4-[4-(4-oxo-4,5,6,7-tetrahydro-1H-pyrrolo[3,2- c]pyridin-2-yl)pyridin-2-yl]benzenesulfonamide trifluoroacetate | 0.12 | |
| 471 | 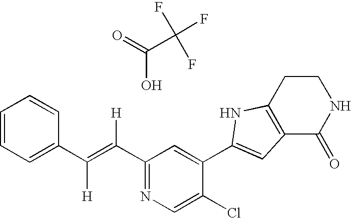 | 2-{5-chloro-2-[(E)-2-phenylvinyl]pyridin-4-yl}-1,5,6,7-tetrahydro- 4H-pyrrolo[3,2-c]pyridin-4-one trifluoroacetate | 0.12 | |
| 472 | 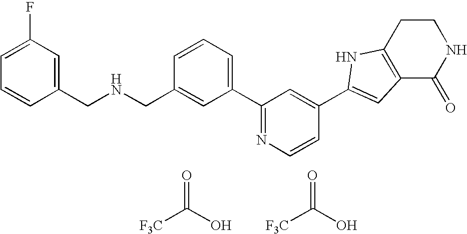 | 2-[2-(3-{[(3-fluorobenzyl)amlno]methyl}phenyl)pyridin-4-yl]- 1,5,6,7-tetrahydro-4H-pyrrolo[3,2-c]pyridin-4-one bis(trifluoroacetate) | 0.123 | |
| 473 | 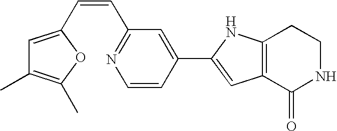 | 2-{2-[(Z)-2-(4,5-dimethyl-2-furyl)vinyl]pyridin-4-yl}-1,5,6,7- tetrahydro-4H-pyrrolo[3,2-c]pyridin-4-one | 0.124 | |
| 474 | 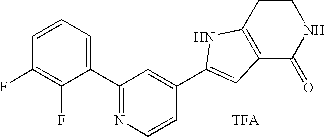 | 2-[2-(2,3-difluorophenyl)pyridin-4-yl]-1,5,6,7-tetrahydro-4H- pyrrolo[3,2-c]pyridin-4-one trifluoroacetate | 0.125 | |
| 475 | 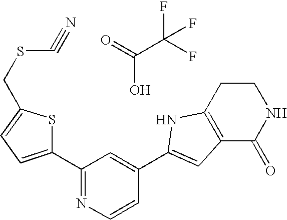 | {5-[4-(4-oxo-4,5,6,7-tetrahydro-1H-pyrrolo[3,2-c]pyridin-2- yl)pyridin-2-yl]thien-2-yl}methyl thiocyanate trifluoroacetate | 0.125 | |
| 476 | 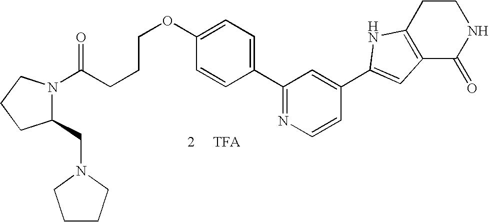 | 2-[2-(4-{4-oxo-4-[(2R)-2-(pyrrolidin-1-ylmethyl)pyrrolidin-1- yl]butoxylphenyl)pyridin-4-yl]-1,5,6,7-tetrahydro-4H-pyrrolo[3,2- c]pyridin-4-one trifluoroacetate | 0.126 | |
| 477 | 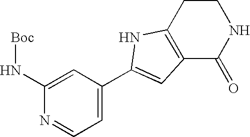 | tert-butyl 4-(4-axo-4,5,6,7-tetrahydro-1H-pyrrolo[3,2-c]pyridmn-2- yl)pyridin-2-ylcarbamate | 0.126 | |
| 478 | 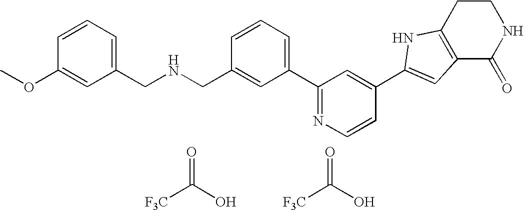 | 2-[2-(3-{[(3-methoxybenzyl)amino]methyl}phenyl)pyridin-4-yl]- 1,5,6,7-tetrahydro-4H-pyrrolo[3,2-c]pyridin-4-one bis(trifluoroacetate) | 0.127 | |
| 479 | 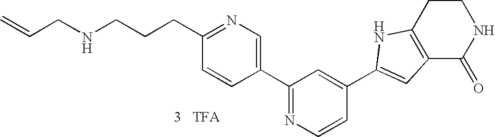 | 2-{6′-[3-(aHylamino)propyll-2,3′-bipyridin-4-yl}-1,5,6,7-tetrahydro- 4H-pyrrolo[3,2-c]pyridin-4-one trifluoroacetate | 0.127 | |
| 480 | 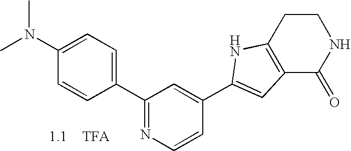 | 2-{2-[4-(dimethylamino)phenyl]pyridin-4-yl}-1,5,6,7-tetrahydro- 4H-pyrrolo[3,2-c]pyridin-4-one trifluoroacetate | 0.128 | |
| 481 | 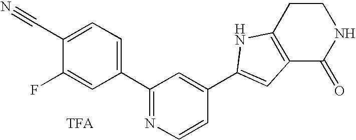 | 2-fluoro-4-[4-(4-oxo-4,5,6,7-tetrahydro-1H-pyrrolo[3,2-c]pyridin- 2-yl)pyridin-2-yl]benzonitrile | 0.129 | |
| 482 | 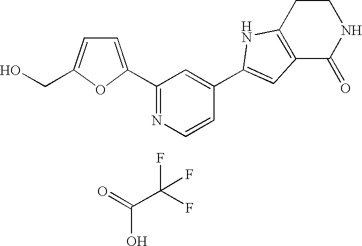 | 2-{2-[5-(hydroxymethyl)-2-furyl]pyridin-4-yl}-1,5,6,7-tetrahydro- 4H-pyrrolo[3,2-c]pyridin-4-one trifluoroacetate | 0.129 | |
| 483 | 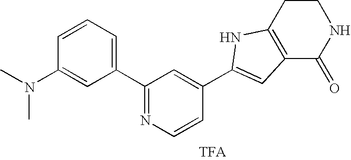 | 2-{2-[3-(dimethylamino)phenyl]pyridin-4-yl}-1,5,6,7-tetrahydro- 4H-pyrrolo[3,2-c]pyridin-4-one trifluoroacetate | 0.131 | |
| 484 | 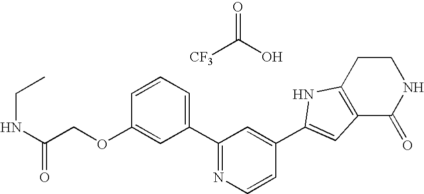 | N-ethyl-2-{3-[4-(4-oxo-4,56,7-tetrahydro-1H-pyrrolo[3,2- c]pyridin-2-yl)pyridin-2-yl]phenoxy}acetamide trifluoroacetate | 0.131 | |
| 485 | 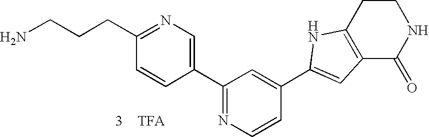 | 2-[6′-(3-aminopropyl)-2,3′-bipyridin-4-yl]-1,5,6,7-tetrahydro-4H- pyrrolo[3,2-c]pyridin-4-one trifluoroacetate | 0.132 | |
| 486 | 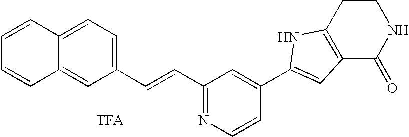 | 2-{2-[(E)-2-(2-naphthyl)vinyl]pyridin-4-yl}-1,5,6,7-tetrahydro-4H- pyrrolo[3,2-c]pyridin-4-one trifluoroacetate | 0.132 | |
| 487 | 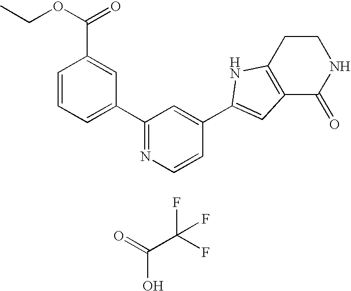 | ethyl 3-[4-(4-oxo-4,5,6,7-tetrahydro-1H-pyrrolo[3,2-c]pyridin-2- yl)pyridin-2-yl]benzoate trifluoroacetate | 0.133 | |
| 488 | 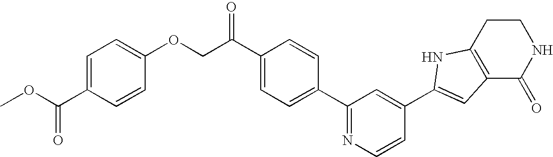 | methyl 4-(2-oxo-2-{4-[4-(4-oxo-4,5,6,7-tetrahydro-1H- pyrrolo[3,2-c]pyridin-2-yl)pyridin-2-yl]phenyl}ethoxy)benzoate | 0.133 | |
| 489 | 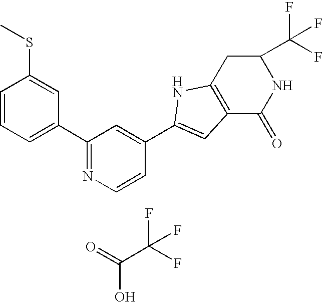 | 2-{2-[3-(methylthio)phenyl]pyridin-4-yl}-6-(trifluoromethyl)- 1,5,6,7-tetrahydro-4H-pyrrolo[3,2-c]pyridin-4-one trifluoroacetate | 0.133 | |
| 490 | 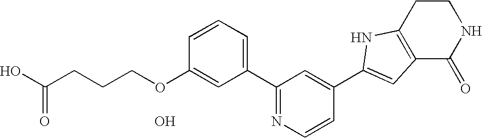 | 4-{3-[4-(4-oxo-4,5,6,7-tetrahydro-1H-pyrrolo[3,2-c]pyridin-2- yl)pyridin-2-yl]phenoxy}butanoic acid hydrochloride | 0.133 | |
| 491 | 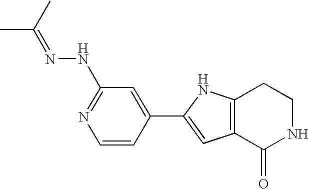 | 2-{2-[2-(1-methylethylidene)hydrazino]pyridin-4-yl}-1,5,6,7- tetrahydro-4H-pyrrolo[3,2-c]pyridin-4-one | 0.133 | |
| 492 | 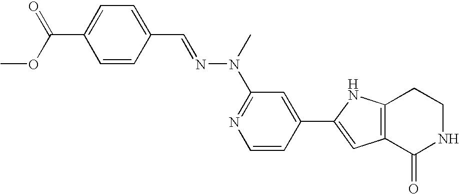 | methyl 4-((E)-{methyl[4-(4-oxo-4,5,6,7-tetrahydro-1H- pyrrolo[3,2-c]pyridin-2-yl)pyridin-2- yl]hydrazono}methyl)benzoate | 0.133 | |
| 493 | 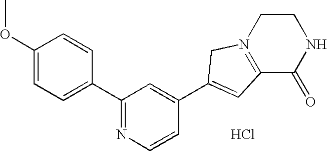 | 7-[2-(4-methoxyphenyl)pyridin-4-yl]-3,4-dihydropyrrolo[1,2- a]pyrazin-1(2H)-one hydrochloride | 0.134 | |
| 494 | 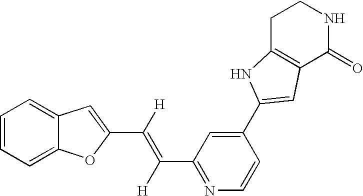 | 2-{2-[(E)-2-(1-benzofuran-2-yl)vinyl]pyridin-4-yl}-1,5,6,7- tetrahydro-4H-pyrrolo[3,2-c]pyridin-4-one | 0.134 | |
| 495 | 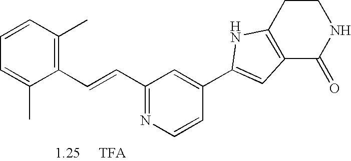 | 2-{2-[(E)-2-(2,6-dimethylphenyl)vinyl]pyridin-4-yl}-1,5,6,7- tetrahydro-4H-pyrrolo[3,2-c]pyridin-4-one trifluoroacetate | 0.134 | |
| 496 | 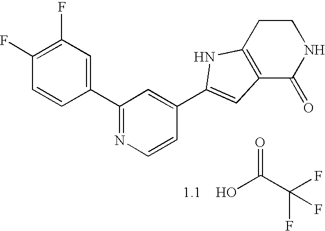 | 2-[2-(3,4-difluorophenyl)pyridin-4-yl]-1,5,6,7-tetrahydro-4H- pyrrolo[3,2-c]pyridin-4-one trifluoroacetate | 0.136 | |
| 497 | 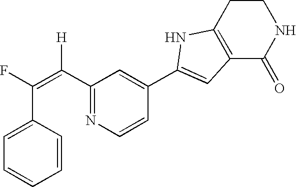 | 2-{2-[(E)-2-fluoro-2-phenylvinyl]pyridin-4-yl}-1,5,6,7-tetrahydro- 4H-pyrrolo[3,2-c]pyridin-4-one | 0.136 | |
| 498 | 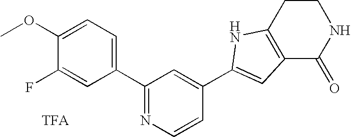 | 2-{2-(3-fluoro-4-methoxyphenyl)pyridin-4-yl]-1,5,6,7-tetrahydro- 4H-pyrrolo[3,2-c]pyridin-4-one trifluoroacetate | 0.137 | |
| 499 | 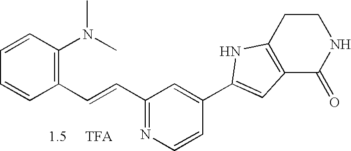 | 2-(2-{(E)-2-[2-(dimethylamino)phenyl]vinyl}pyridin-4-yl)-1,5,6,7- tetrahydro-4H-pyrrolo[3,2-c]pyridin-4-one trifluoroacetate | 0.137 | |
| 500 | 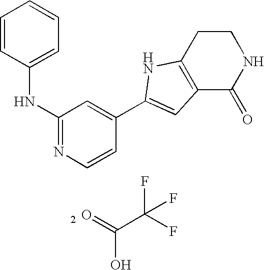 | 2-(2-anilinopyridin-4-yl)-1,5,6,7-tetrahydro-4H-pyrrolo[3,2- c]pyridin-4-one trifluoroacetate | 0.138 | |
| 501 | 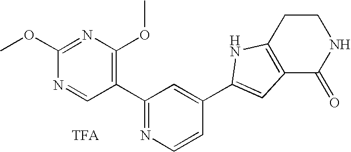 | 2-[2-(2,4-dimethoxypyrimidin-5-yl)pyridin-4-yl]-1,5,6,7- tetrahydro-4H-pyrrolo[3,2-c]pyridin-4-one trifluoroacetate | 0.141 | |
| 502 | 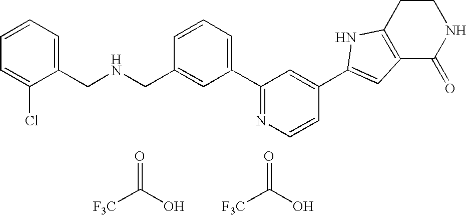 | 2-[2-(3-{[(2-chlorobenzyl)amino]methyl}phenyl)pyridin-4-yl]- 1,5,6,7-tetrahydro-4H-pyrrolo[3,2-c]pyridin-4-one bis(trifluoroacetate) | 0.142 | |
| 503 | 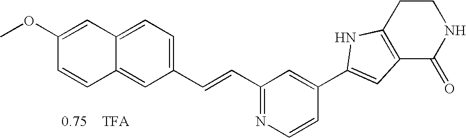 | 2-{2-[(E)-2-(6-methoxy-2-naphthyl)vinyl]pyridin-4-yl}-1,5,6,7- tetrahydro-4H-pyrrolo[3,2-c]pyridin-4-one trifluoroacetate | 0.143 | |
| 504 | 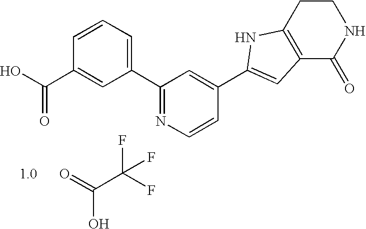 | 3-[4-(4-oxo-4,5,6,7-tetrahydro-1H-pyrrolo[3,2-c]pyridin-2- yl)pyridin-2-yl]benzoic acid trifluoroacetate | 0.145 | |
| 505 | 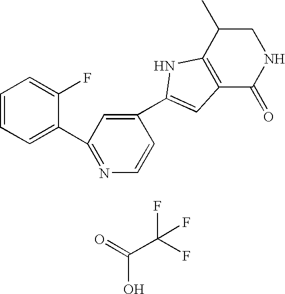 | 2-[2-(2-fluorophenyl)pyridin-4-yl]-7-methyl-1,5,6,7-tetrahydro-4H- pyrrolo[3,2-c]pyridin-4-one trifluoroacetate | 0.145 | |
| 506 | 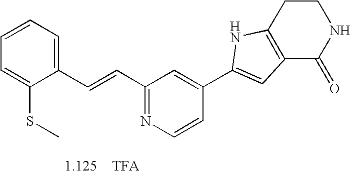 | 2-(2-{(E)-2-[2-(methylthio)phenyl]vinyl}pyridin-4-yl)-1,5,6,7- tetrahydro-4H-pyrrolo[3,2-c]pyridin-4-one trifluoroacetate | 0.145 | |
| 507 | 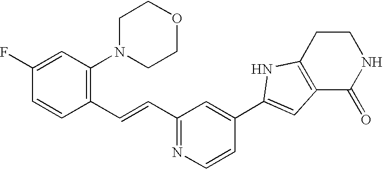 | 2-{2-[(E)-2-(4-fluoro-2-morpholin-4-ylphenyl)vinyl]pyridin-4-yl}- 1,5,6,7-tetrahydro-4H-pyrrolo[3,2-c]pyridin-4-one | 0.148 | |
| 508 | 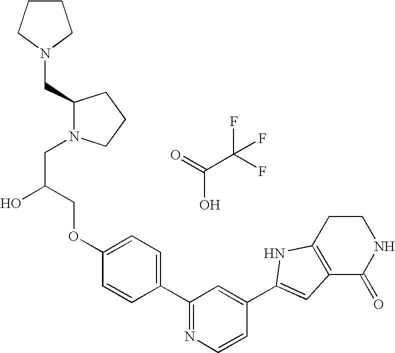 | 2-[2-(4-{2-hydroxy-3-[(2R)-2-(pyrrolidin-1-ylmethyl)pyrrolidin-1- yl]propoxy}phenyl)pyridin-4-yl]-1,5,6,7-tetrahydro-4H-pyrrolo[3,2- c]pyridin-4-one trifluoroacetate | 0.149 | |
| 509 | 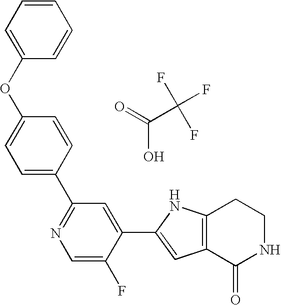 | 2-[5-fluoro-2-(4-phenoxyphenyl)pyridin-4-yl]-1,5,6,7-tetrahydro- 4H-pyrrolo[3,2-c]pyridin-4-one trifluoroacetate | 0.149 | |
| 510 | 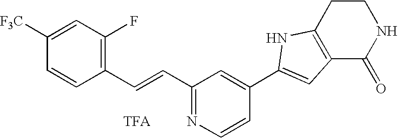 | 2-(2-{(E)-2-[2-fluoro-4-(trifluoromethyl)phenyl]vinyl}pyridin-4-yl)- 1,5,6,7-tetrahydro-4H-pyrrolo[3,2-c]pyridin-4-one trifluoroacetate | 0.15 | |
| 511 | 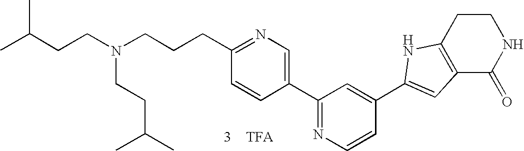 | 2-(6′-{3-(bis(3-methylbutyl)amino]propyl}-2,3′-bipyridin-4-yl)- 1,5,6,7-tetrahydro-4H-pyrrolo[3,2-c]pyridin-4-one trifluoroacetate | 0.151 | |
| 512 | 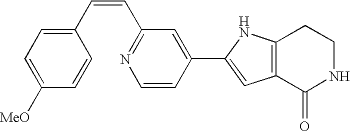 | 2-{2-[(Z)-2-(4-methoxyphenyl)ethenyl]pyridin-4-yl}-1,5,6,7- tetrahydro-4H-pyrrolo[3,2-c]pyridin-4-one | 0.152 | |
| 513 | 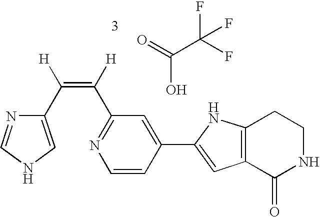 | 2-{2-((Z)-2-(1H-imidazol-4-yl)ethenyl]pyridin-4-yl}-1,5,6,7- tetrahydro-4H-pyrrolo[3,2-c]pyridin-4-one trifluoroacetate | 0.152 | |
| 514 | 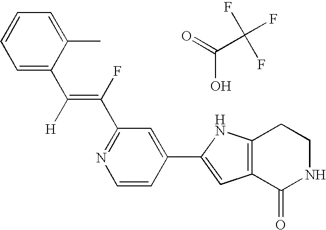 | 2-{2-[(Z)-1-fluoro-2-(2-methylphenyl)vinyl]pyridin-4-yl}-1,5,6,7- tetrahydro-4H-pyrrolo[3,2-c]pyridin-4-one trifluoroacetate | 0.152 | |
| 515 | 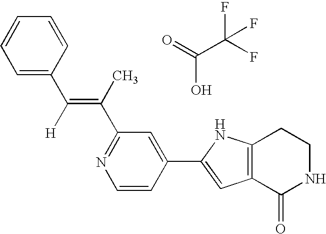 | 2-{2-[(E)-1-methyl-2-phenylethenyl]pyridin-4-yl}-1,5,6,7- tetrahydro-4H-pyrrolo[3,2-c]pyridin-4-one trifluoroacetate | 0.154 | |
| 516 | 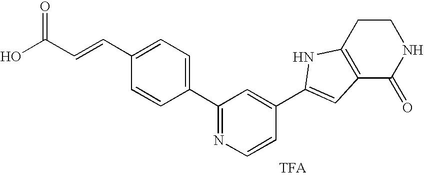 | (2E)-3-{4-[4-(4-oxo-4,5,6,7-tetrahydro-1H-pyrrolo[3,2-c]pyridin-2- yl)pyridin-2-yl]phenyl}prop-2-enoic acid trifluoroacetate | 0.155 | |
| 517 | 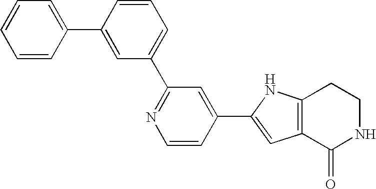 | 2-[2-(1,1′-biphenyl-3-yl)pyridin-4-yl]-1,5,6,7-tetrahydro-4H- pyrrolo[3,2-c]pyridin-4-one | 0.155 | |
| 518 | 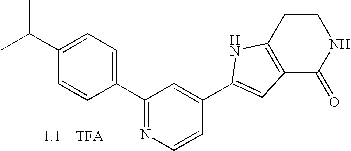 | 2-[2-(4-isopropylphenyl)pyridin-4-yl]-1,5,6,7-tetrahydro-4H- pyrrolo[3,2-c]pyridin-4-one trifluoroacetate | 0.156 | |
| 519 | 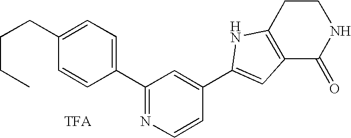 | 2-[2-(4-butylphenyl)pyridin-4-yl]-1,5,6,7-tetrahydro-4H- pyrrolo[3,2-c]pyridin-4-one trifluoroacetate | 0.156 | |
| 520 | 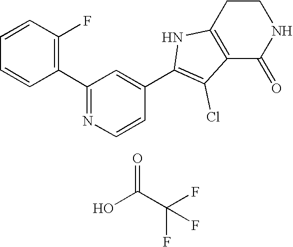 | 3-chloro-2-[2-(2-fluorophenyl)pyridin-4-yl]-1,5,6,7-tetrahydro-4H- pyrrolo[3,2-c]pyridin-4-one trifluoroacetate | 0.156 | |
| 521 | 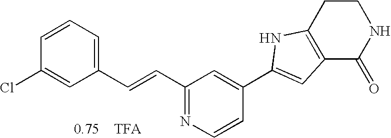 | 2-{2-[(E)-2-(3-chlorophenyl)vinyl]pyridin-4-yl}-1,5,6,7-tetrahydro- 4H-pyrrolo[3,2-c]pyridin-4-one trifluoroacetate | 0.157 | |
| 522 | 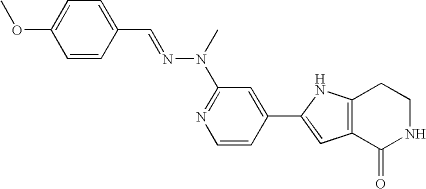 | 4-methoxybenzaldehyde methyl[4-(4-oxo-4,5,6,7-tetrahydro-1H- pyrrolo[3,2-c]pyridin-2-yl)pyridin-2-yl]hydrazone | 0.157 | |
| 523 | 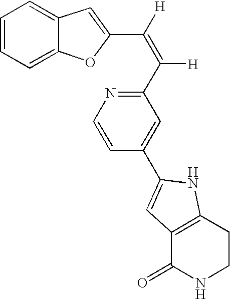 | 2-{2-[(Z)-2-(1-benzofuran-2-yl)vinyl]pyridin-4-yl}-1,5,6,7- tetrahydro-4H-pyrrolo[3,2-c]pyridin-4-one | 0.163 | |
| 524 | 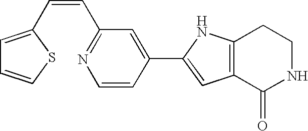 | 2-{2-[(Z)-2-thien-2-ylethenyl]pyridin-4-yl}-1,5,6,7-tetrahydro-4H- pyrrolo[3,2-c]pyridin-4-one | 0.164 | |
| 525 | 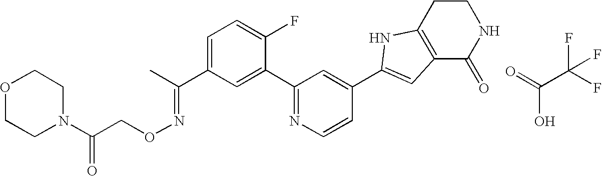 | 2-(2-{2-fluoro-5-[(1E)-N-(2-morpholin-4-yl-2- oxoethoxy)ethanimidoyl]phenyl}pyridin-4-yl)-1,5,6,7-tetrahydro- 4H-pyrrolo[3,2-c]pyridin-4-one trifluoroacetate | 0.165 | |
| 526 | 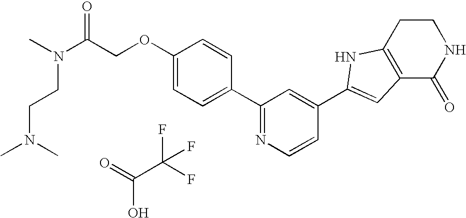 | N-[2-(dimethylamino)ethyl]-N-methyl-2-{4-[4-(4-oxo-4,5,6,7- tetrahydro-1H-pyrrolo[3,2-c]pyridin-2-yl)pyridin-2- yl]phenoxy}acetamide trifluoroacetate | 0.166 | |
| 527 | 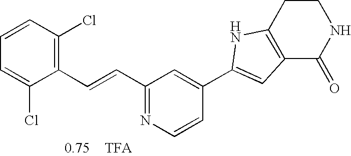 | 2-{2-[(E)-2-(2,6-dichlorophenyl)vinyl]pyridin-4-yl}-1,5,6,7- tetrahydro-4H-pyrrolo[3,2-c]pyridin-4-one trifluoroacetate | 0.169 | |
| 528 | 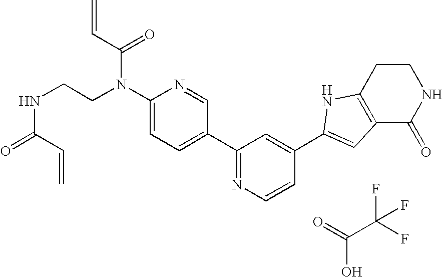 | N-[2-(acryloylamino)ethyl]-N-[4-(4-oxo-4,5,6,7-tetrahydro-1H- pyrrolo[3,2-c]pyridin-2-yl)-2,3-bipyridin-6′-yl]acrylamide trifluoroacetate | 0.171 | |
| 529 | 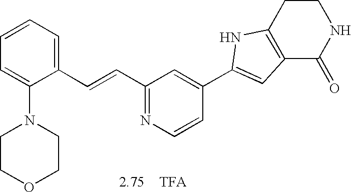 | 2-{2-[(E)-2-(2-morpholin-4-ylphenyl)vinyl]pyridin-4-yl}-1,5,6,7- tetrahydro-4H-pyrrolo[3,2-c]pyridin-4-one trifluoroacetate | 0.171 | |
| 530 | 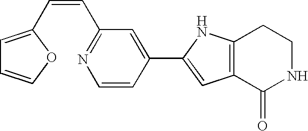 | 2-{2-[(Z)-2-(2-furyl)vinyl]pyridin-4-yl}-1,5,6,7-tetrahydro-4H- pyrrolo[3,2-c]pyridin-4-one | 0.172 | |
| 531 | 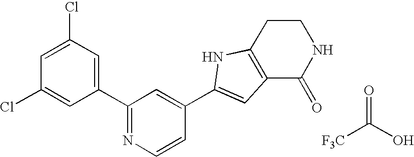 | 2-[2-(3,5-dichlorophenyl)pyridin-4-yl]-1,5,6,7-tetrahydro-4H- pyrrolo[3,2-c]pyridin-4-one trifluoroacetate | 0.173 | |
| 532 | 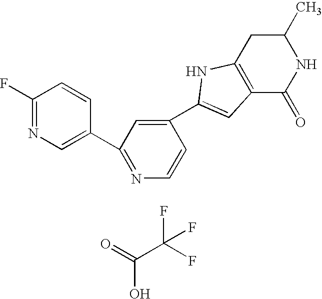 | 2-(6′-fluoro-2,3-bipyridin-4-yl)-6-methyl-1,5,6,7-tetrahydro-4H- pyrrolo[3,2-c]pyridin-4-one trifluoroacetate | 0.173 | |
| 533 | 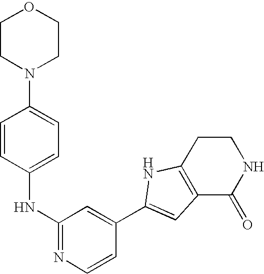 | 2-{2-[(4-morpholin-4-ylphenyl)amino]pyridin-4-yl}-1,5,6,7- tetrahydro-4H-pyrrolo[3,2-c]pyridin-4-one | 0.173 | |
| 534 | 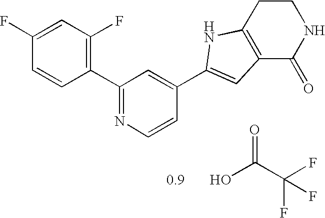 | 2-[2-(2,4-difluorophenyl)pyridin-4-yl]-1,5,6,7-tetrahydro-4H- pyrrolo[3,2-c]pyridin-4-ane trifluoroacetate | 0.179 | |
| 535 | 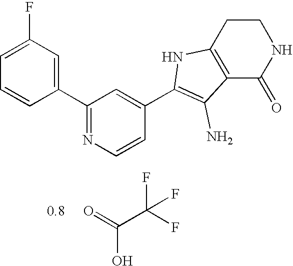 | 3-amino-2-[2-(3-fluorophenyl)pyridin-4-yl]-1,5,6,7-tetrahydro-4H- pyrrolo[3,2-c]pyridin-4-one trifluoroacetate | 0.183 | |
| 536 | 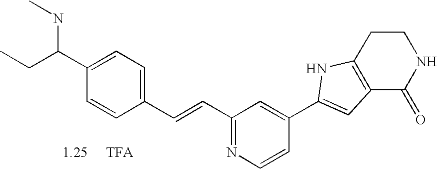 | 2-(2-{(E)-2-[4-(diethylamino)phenyl]vinyl}pyridin-4-yl)-1,5,6,7- tetrahydro-4H-pyrrolo[3,2-c]pyridin-4-one trifluoroacetate | 0.187 | |
| 537 | 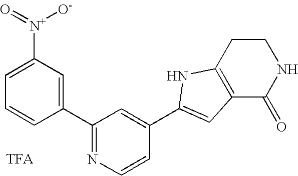 | 2-[2-(3-nitrophenyl)pyridin-4-yl]-1,5,6,7-tetrahydro-4H- pyrrolo[3,2-c]pyridin-4-one trifluoroacetate | 0.188 | |
| 538 | 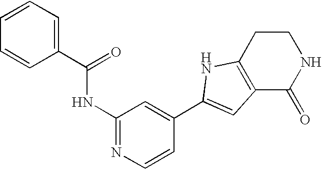 | N-[4-(4-oxo-4,5,6,7-tetrahydro-1H-pyrrolo[3,2-c]pyridin-2- yl)pyridin-2-yl]benzamide | 0.188 | |
| 539 | 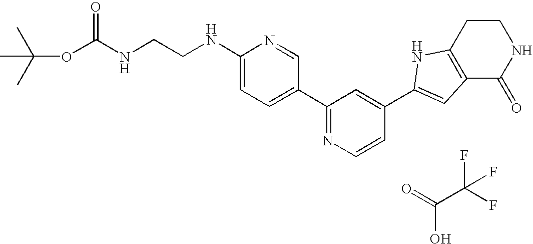 | tert-butyl 2-{[4-(4-oxo-4,5,6,7-tetrahydro-1H-pyrrolo[3,2- c]pyridin-2-yl)-2,3′-bipyridin-6′-yl]amino}ethylcarbamate trifluoroacetate | 0.189 | |
| 540 | 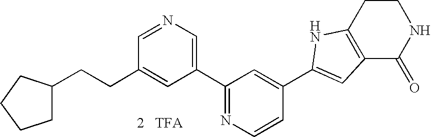 | 2-(5′-(2-cyclopentylethyl)-2,3′-bipyridin-4-yl]-1,5,6,7-tetrahydro- 4H-pyrrolo[3,2-c]pyridin-4-one trifluoroacetate | 0.189 | |
| 541 | 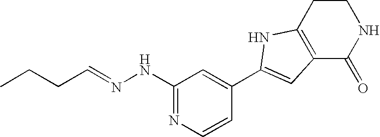 | (E)-butanal [4-(4-oxo-4,5,6,7-tetrahydro-1H-pyrrolo[3,2- c]pyridin-2-yl)pyridin-2-yl]hydrazone | 0.19 | |
| 542 | 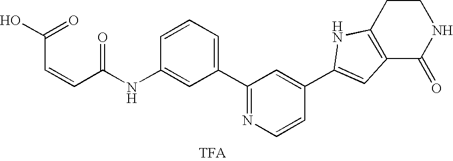 | (2Z)-4-oxo-4-({3-[4-(4-oxo-4,5,6,7-tetrahydro-1H-pyrrolo[3,2- c]pyridin-2-yl)pyridin-2-yl]phenyl}amino)but-2-enoic acid trifluoroacetate | 0.193 | |
| 543 | 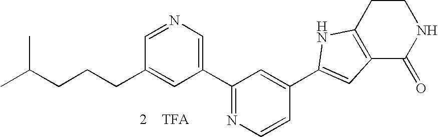 | 2-[5′-(4-methylpentyl)-2,3′-bipyridin-4-yl]-1,5,6,7-tetrahydro-4H- pyrrolo[3,2-c]pyridin-4-one trifluoroacetate | 0.194 | |
| 544 | 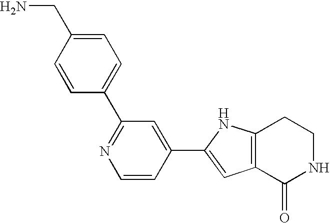 | 2-{2-[4-(aminomethyl)phenyl]pyridin-4-yl)-1,5,6,7-tetrahydro-4H- pyrrolo[3,2-c]pyridin-4-one | 0.195 | |
| 545 | 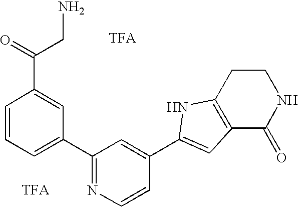 | 2-{2-[3-(aminoacetyl)phenyl]pyridin-4-yl)-1,5,6,7-tetrahydro-4H- pyrrolo[3,2-c]pyridin-4-one bis(trifluoroacetate) | 0.195 | |
| 546 | 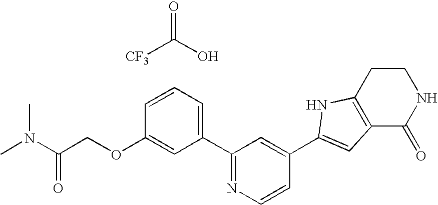 | N,N-dimethyl-2-}3-[4-(4-oxo-4,5,6,7-tetrahydro-1H-pyrrolo[3,2- c]pyridin-2-yl)pyridin-2-yl]phenoxy}acetamide trifluoroacetate | 0.197 | |
| 547 | 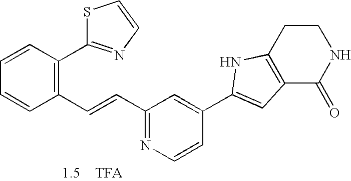 | 2-(2-{(E)-2-[2-(1,3-thiazol-2-yl)phenyl]vinyl}pyridin-4-yl)-1,5,6,7- tetrahydro-4H-pyrrolo[3,2-c]pyridin-4-one trifluoroacetate | 0.197 | |
| 548 | 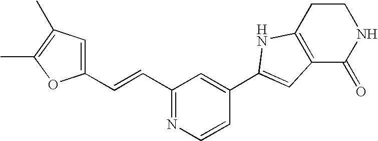 | 2-{2-[(E)-2-(4,5-dimethyl-2-furyl)vinyl]pyridin-4-yl}-1,5,6,7- tetrahydro-4H-pyrrolo[3,2-c]pyridin-4-one | 0.201 | |
| 549 | 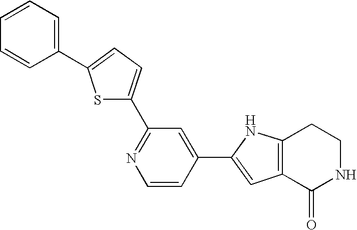 | 2-[2-(5-phenylthien-2-yl)pyridin-4-yl]-1,5,6,7-tetrahydro-4H- pyrrolo[3,2-c]pyridin-4-one | 0.202 | |
| 550 | 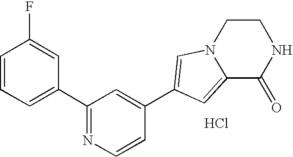 | 7-[2-(3-fluorophenyl)pyridin-4-yl]-3,4-dihydropyrrolo[1,2- a]pyrazin-1(2H)-one hydrochloride | 0.205 | |
| 551 | 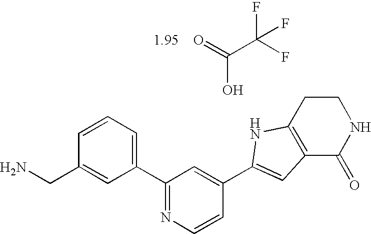 | 2-{2-[3-(aminomethyl)phenyl]pyridin-4-yl}-1,5,6,7-tetrahydro-4H- pyrrolo[3,2-c]pyridin-4-one trifluoroacetate | 0.207 | |
| 552 | 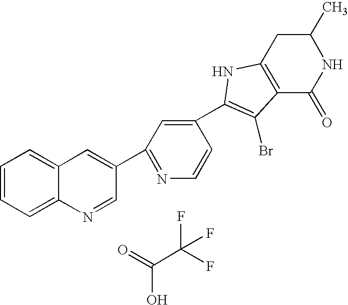 | 3-bromo-6-methyl-2-(2-quinolin-3-ylpyridin-4-yl)-1,5,6,7- tetrahydro-4H-pyrrolo[3,2-c]pyridin-4-one trifluoroacetate | 0.215 | |
| 553 | 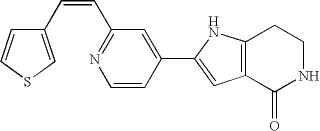 | 2-{2-[(Z)-2-thien-3-ylvinyl]pyridin-4-yl}-1,5,6,7-tetrahydro-4H- pyrrolo[3,2-c]pyridin-4-one | 0.22 | |
| 554 | 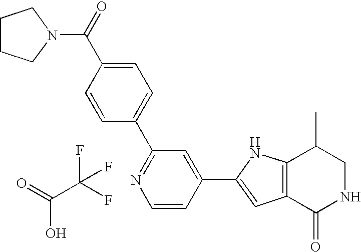 | 7-methyl-2-{2-[4-(pyrrolidin-1-ylcarbonyl)phenyl]pyridin-4-yl}- 1,5,6,7-tetrahydro-4H-pyrrolo[3,2-c]pyridin-4-one trifluoroacetate | 0.225 | |
| 555 | 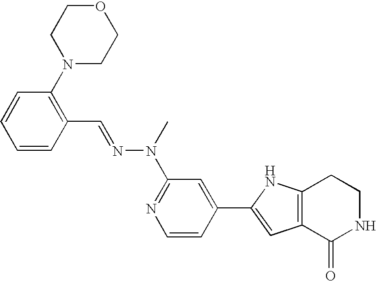 | 2-morpholin-4-ylbenzaldehyde methyl[4-(4-oxo-4,5,6,7- tetrahydro-1H-pyrrolo[3,2-c]pyridin-2-yl)pyridin-2-yl]hydrazone | 0.226 | |
| 556 | 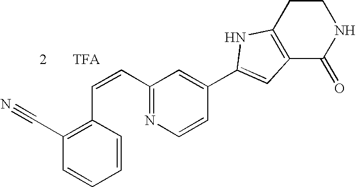 | 2-{(Z)-2-[4-(4-oxo-4,5,6,7-tetrahydro-1H-pyrrolo[3,2-c]pyridin-2- yl)pyridin-2-yl]vinyl}benzonitrile trifluoroacetate | 0.228 | |
| 557 | 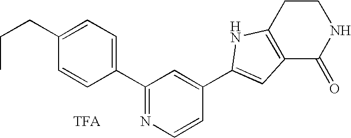 | 2-[2-(4-propylphenyl)pyridin-4-yl]-1,5,6,7-tetrahydro-4H- pyrrolo[3,2-c]pyridin-4-one trifluoroacetate | 0.231 | |
| 558 | 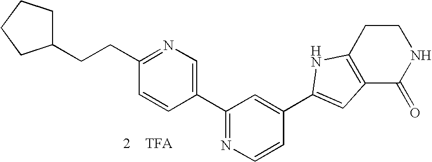 | 2-[6′-(2-cyclopentylethyl)-2,3′-bipyridin-4-yl]-1,5,6,7-tetrahydro- 4H-pyrrolo[3,2-c]pyridin-4-one trifluoroacetate | 0.234 | |
| 559 | 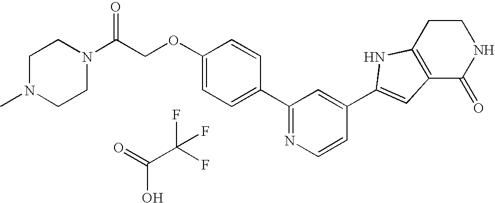 | 2-(2-{4-[2-(4-methylpiperazin-1-yl)-2-oxoethoxy]phenyl)pyridin-4- yl)-1,5,6,7-tetrahydro-4H-pyrrolo[3,2-c]pyridin-4-one trifluoroacetate | 0.236 | |
| 560 | 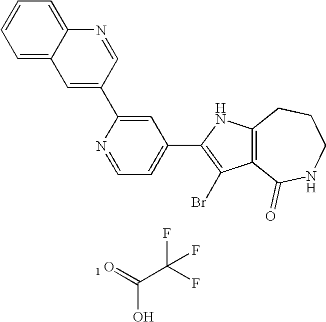 | 3-bromo-2-(2-quinolin-3-ylpyridin-4-yl)-5,6,7,8- tetrahydropyrrolo[3,2-c]azepin-4(1H)-one trifluoroacetate | 0.242 | |
| 561 | 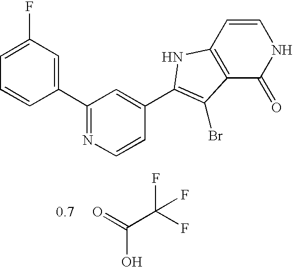 | 3-bromo-2-[2-(3-fluorophenyl)pyridin-4-yl]-1,5-dihydro-4H- pyrrolo[3,2-c]pyridin-4-one trifluoroacetate | 0.244 | |
| 562 | 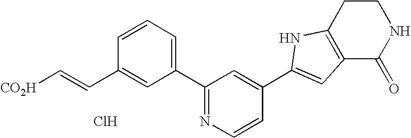 | (2E)-3-{3-[4-(4-oxo-4,5,6,7-tetrahydro-1H-pyrrolo[3,2-c]pyridin-2- yl)pyridin-2-yl]phenyl}acrylic acid hydrochloride | 0.245 | |
| 563 | 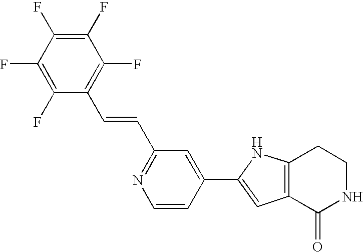 | 2-{2-[(E)-2-(pentafluorophenyl)ethenyl]pyridin-4-yl}-1,5,6,7- tetrahydro-4H-pyrrolo[3,2-c]pyridin-4-one | 0.245 | |
| 564 | 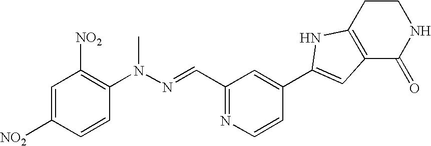 | 4-(4-oxo-4,5,6,7-tetrahydro-1H-pyrrolo[3,2-c]pyridin-2- yl)pyridine-2-carbaldehyde (2,4- dinitrophenyl)(methyl)hydrazone | 0.245 | |
| 565 | 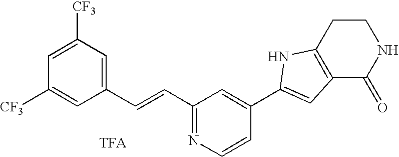 | 2-(2-{(E)-2-[3,5-bis(trifluoromethyl)phenyl]vinyl}pyridin-4-yl)- 1,5,6,7-tetrahydro-4H-pyrrolo[3,2-c]pyridin-4-one trifluoroacetate | 0.247 | |
| 566 | 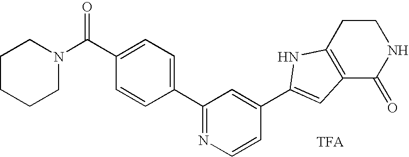 | 2-{2-[4-(pipendin-1-ylcarbonyl)phenyl]pyridin-4-yl}-1,5,6,7- tetrahydro-4H-pyrrolo[3,2-c]pyridin-4-one trifluoroacetate | 0.251 | |
| 567 | 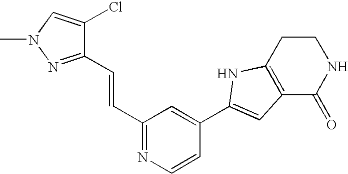 | 2-{2-[(E)-2-(4-chloro-1-methyl-1H-pyrazol-3-yl)vinyl]pyridin-4-yl}- 1,5,6,7-tetrahydro-4H-pyrrolo[3,2-c]pyridin-4-one | 0.251 | |
| 568 | 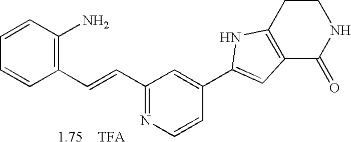 | 2-{2-[(E)-2-(2-aminophenyl)vinyl]pyridin-4-yl}-1,5,6,7-tetrahydro- 4H-pyrrolo[3,2-c]pyridin-4-one trifluoroacetate | 0.255 | |
| 569 | 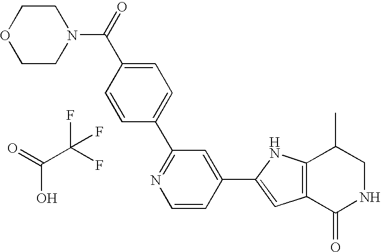 | 7-methyl-2-{2-[4-(morpholin-4-ylcarbonyl)phenyl]pyridin-4-yl}- 1,5,6,7-tetrahydro-4H-pyrrolo[3,2-c]pyridin-4-one trifluoroacetate | 0.256 | |
| 570 | 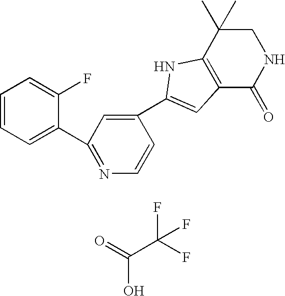 | 2-[2-(2-fluorophenyl)pyridin-4-yl]-7,7-dimethyl-1,5,6,7-tetrahydro- 4H-pyrrolo[3,2c]-pyridin-4-one trifluoroacetate | 0.259 | |
| 571 | 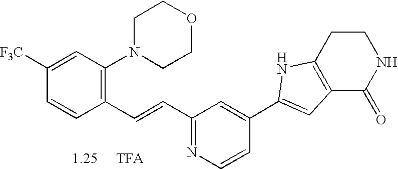 | 2-(2-{(E)-2-[2-morpholin-4-yl-4- (trifluoromethyl)phenyl]vinyl}pyridin-4-yl)-1,5,6,7-tetrahydro-4H- pyrrolo[3,2-c]pyridin-4-one trifluoroacetate | 0.266 | |
| 572 | 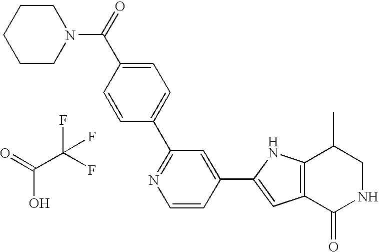 | 7-methyl-2-{2-[4-(piperidin-1-ylcarbonyl)phenyl]pyridin-4-yl}- 1,5,6,7-tetrahydro-4H-pyrrolo[3,2-c]pyridin-4-one trifluoroacetate | 0.271 | |
| 573 | 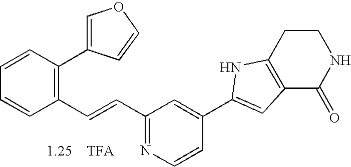 | 2-(2-{(E)-2-[2-(3-furyl)phenyl]vinyl}pyridin-4-yl)-1,5,6,7- tetrahydro-4H-pyrrolo[3,2-c]pyridin4-one trifluoroacetate | 0.271 | |
| 574 | 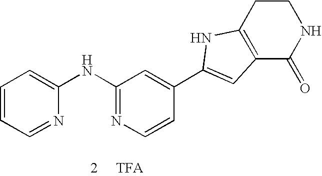 | 2-[2-(pyridin-2-ylamino)pyridin-4-yl]-1,5,6,7-tetrahydro-4H- pyrrolo[3,2-c]pyridin-4-one trifluoroacetate | 0.271 | |
| 575 | 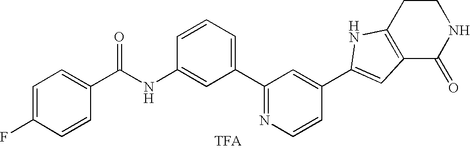 | 4-fluoro-N-{3-[4-(4-oxo-4,5,6,7-tetrahydro-1H-pyrrolo[3,2- c]pyridin-2-yl)pyridin-2-yl]phenyl}benzamide trifluoroacetate | 0.272 | |
| 576 | 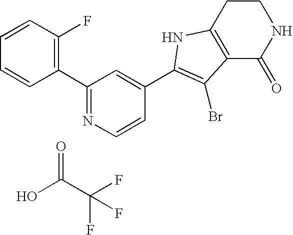 | 3-bromo-2-[2-(2-fluorophenyl)pyridin-4-yl]-1,5,6,7-tetrahydro-4H- pyrrolo[3,2-c]pyridin-4-one trifluoroacetate | 0.279 | |
| 577 | 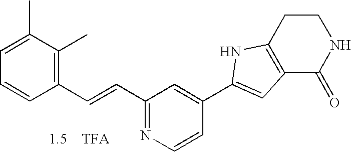 | 2-{2-[(E)-2-(2,3-dimethylphenyl)vinyl]pyridin-4-yl}-1,5,6,7- tetrahydro-4H-pyrrolo[3,2-c]pyridin-4-one trifluoroacetate | 0.279 | |
| 578 | 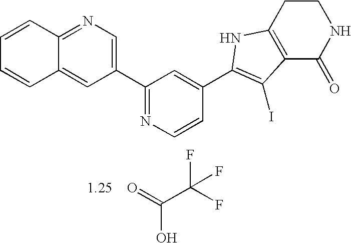 | 3-iodo-2-(2-quinolin-3-ylpyridin-4-yl)-1,5,6,7-tetrahydro-4H- pyrrolo[3,2-c]pyridin-4-one trifiuoroacetate | 0.281 | |
| 579 | 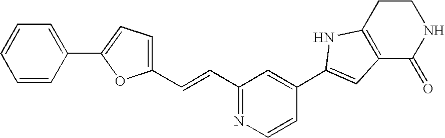 | 2-{2-[(E)-2-(5-phenyl-2-furyl)vinyl]pyridin-4-yl}-1,5,6,7- tetrahydro-4H-pyrrolo[3,2-c]pyridin-4-one | 0.285 | |
| 580 | 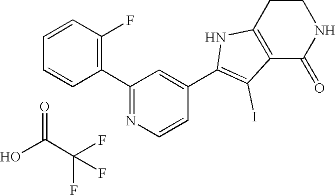 | 2-[2-(2-fluorophenyl)pyridin-4-yl]-3-iodo-1,5,6,7-tetrahydro-4H- pyrrolo[3,2-c]pyridin-4-one trifluoroacetate | 0.29 | |
| 581 | 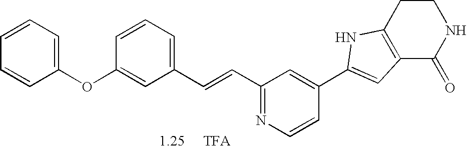 | 2-{2-[(E)-2-(3-phenoxyphenyl)vinyl]pyridin-4-yl}-1,5,6,7- tetrahydro-4H-pyrrolo[3,2-c]pyridin-4-one trifluoroacetate | 0.295 | |
| 582 | 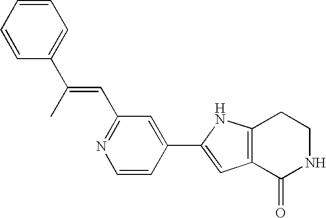 | 2-{2-[(1E)-2-phenylprop-1-enyl]pyridin-4-yl}-1,5,6,7-tetrahydro- 4H-pyrrolo[3,2-c]pyridin-4-one | 0.297 | |
| 583 | 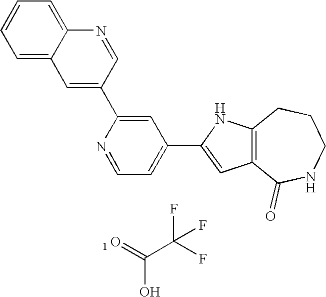 | 2-(2-quinolin-3-ylpyridin-4-yl)-5,6,7,8-tetrahydropyrrolo[3,2- c]azepin-4(1H)-one trifluoroacetate | 0.302 | |
| 584 | 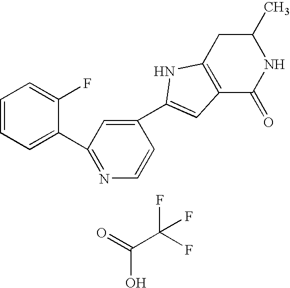 | 2-[2-(2-fluorophenyl)pyridin-4-yl]-6-methyl-1,5,6,7-tetrahydro-4H- pyrrolo[3,2-c]pyridin-4-one trifluoroacetate | 0.302 | |
| 585 | 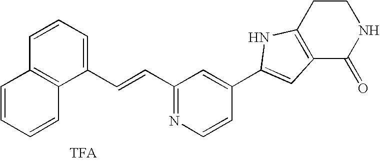 | 2-{2-[(E)-2-(1-naphthyl)vinyl]pyridin-4-yl}-1,5,6,7-tetrahydro-4H- pyrrolo[3,2-c]pyridin-4-one trifluoroacetate | 0.302 | |
| 586 | 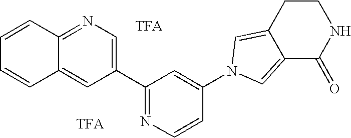 | 2-(2-quinolin-3-ylpyridin-4-yl)-2,5,6,7-tetrahydro-4H-pyrrolo[3,4- c]pyridin-4-one bis(trifluoroacetate) | 0.31 | |
| 587 | 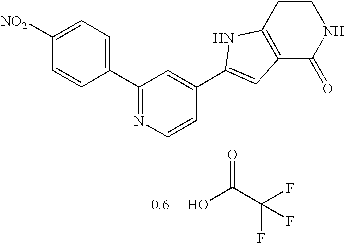 | 2-[2-(4-nitrophenyl)pyridin-4-yl]-1,5,67-tetrahydro-4H- pyrrolo[3,2-c]pyridin-4-one trifluoroacetate | 0.316 | |
| 588 | 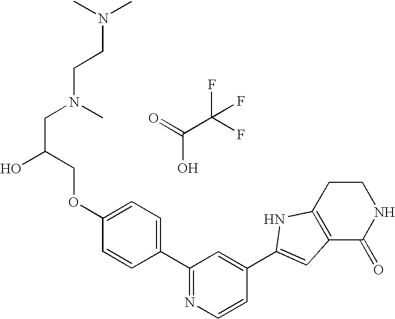 | 2-[2-(4-{3-[[2-(dimethylamino)ethyl](methyl)amino]-2- hydroxypropoxy}phenyl)pyridin-4-yl]-1,5,6,7-tetrahydro-4H- pyrrolo[3,2-c]pyridin-4-one trifluoroacetate | 0.316 | |
| 589 | 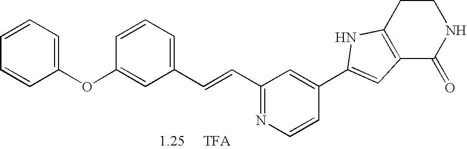 | 2-{2-[(E)-2-(2-fluoro-6-morpholin-4-ylphenyl)vinyl]pyridin-4-yl}- 1,5,6,7-tetrahydro-4H-pyrrolo[3,2-c]pyridin-4-one trifluoroacetate | 0.317 | |
| 590 | 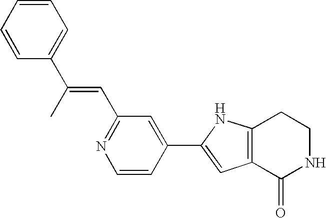 | 2-{2-[3-(morpholin-4-ylmethyl)phenyl]pyridin-4-yl}-1,5,6,7- tetrahydro-4H-pyrrolo[3,2-c]pyridin-4-one trifluoroacetate | 0.319 | |
| 591 | 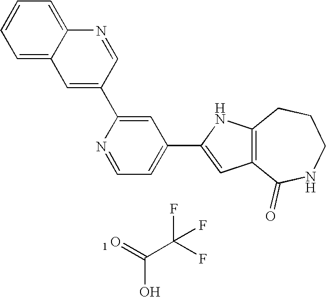 | tert-butyl 5-{(E)-2-[4-(4-oxo-4,5,6,7-tetrahydro-1H-pyrrolo[3,2- c]pyridin-2-yl)pyridin-2-yl]ethenyl}-1H-indole-1-carboxylate trifluoroacetate | 0.319 | |
| 592 | 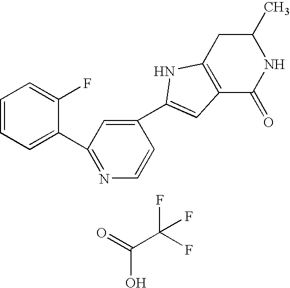 | {4-[4-(4-oxo-4,5,6,7-tetrahydro-1H-pyrrolo[3,2-c]pyridin-2- yl)pyridin-2-yl]phenyl}acetonitrile | 0.322 | |
| 593 | 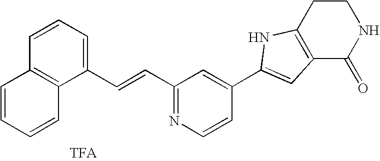 | 2-(2-{(E)-2-[2,4-bis(dimethylamino)phenyl]vinyl}pyridin-4-yl)- 1,5,6,7-tetrahydro-4H-pyrrolo[3,2-c]pyridin-4-one trifluoroacetate | 0.322 | |
| 594 | 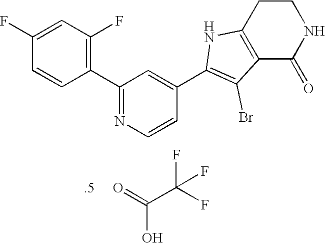 | 3-bromo-2-[2-(2,4-difluorophenyl)pyridin-4-yl]-1,5,6,7-tetrahydro- 4H-pyrrolo[3,2-c]pyridin-4-one trifluoroacetate | 0.333 | |
| 595 | 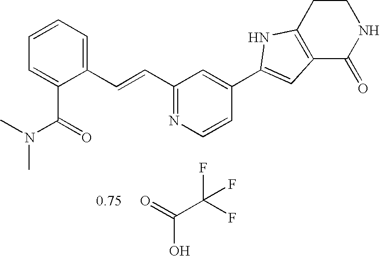 | N,N-dimethyl-2-{(E)-2-[4-(4-oxo-4,5,6,7-tetrahydro-1H- pyrrolo[3,2-c]pyridin-2-yl)pyridin-2-yl]vinyl}benzamide trifluoroacetate | 0.334 | |
| 596 | 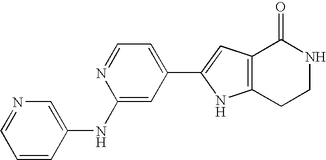 | 2-[2-(pyridin-3-ylamino)pyridin-4-yl]-1,5,6,7-tetrahydro-4H- pyrrolo[3,2-c]pyridin-4-one | 0.336 | |
| 597 | 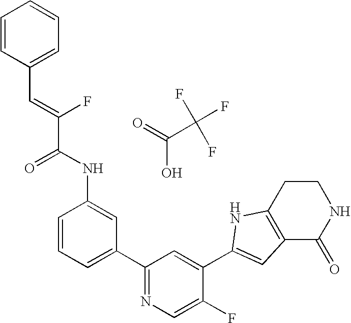 | (2Z)-2-fluoro-N-{3-[5-fluoro-4-(4-oxo-4,5,6,7-tetrahydro-1H- pyrrolo[3,2-c]pyridin-2-yl)pyridin-2-yl]phenyl}-3- phenylacrylamide trifluoroacetate | 0.338 | |
| 598 | 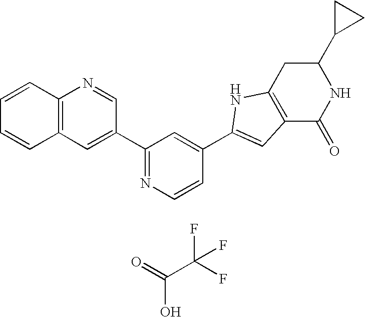 | 6-cyclopropyl-2-(2-quinolin-3-ylpyridin-4-yl)-1,5,6,7-tetrahydro- 4H-pyrrolo[3,2-c]pyridin-4-one trifluoroacetate | 0.341 | |
| 599 | 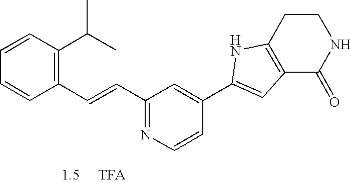 | 2-{2-[(E)-2-(2-isopropylphenyl)vinyl]pyridin-4-yl}-1,5,6,7- tetrahydro-4H-pyrrolo[3,2-c]pyridin-4-one trifluoroacetate | 0.342 | |
| 600 | 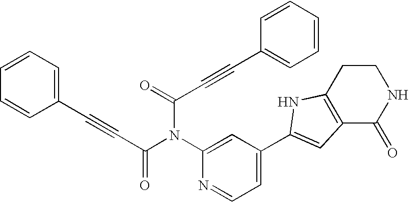 | 2-methyl-N-(4-(4-oxo-4,5,6,7-tetrahydro-1H-pyrrolo[3,2-c]pyridin- 2-yl)pyridin-2-yl]-bis(3-phenylpropynoyl)amide | 0.342 | |
| 601 | 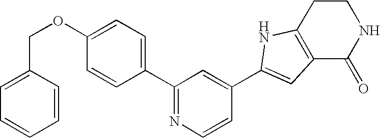 | 2-{2-[4-(benzyloxy)phenyl]pyridin-4-yl}-1,5,6,7-tetrahydro-4H- pyrrolo[3,2-c]pyridin-4-one | 0.348 | |
| 602 | 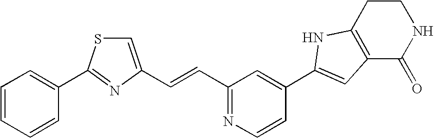 | 2-{2-[(E)-2-(2-phenyl-1,3-thiazol-4-yl)vinyl]pyridin-4-yl}-1,5,6,7- tetrahydro-4H-pyrrolo[3,2-c]pyridin-4-one | 0.348 | |
| 603 | 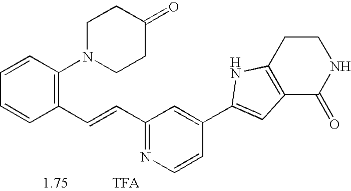 | 2-(2-{(E)-2-[2-(4-oxopipendin-1-yl)phenyl]vinyl}pyridin-4-yl)- 1,5,6,7-tetrahydro-4H-pyrrolo[3,2-c]pyridin-4-one trifluoroacetate | 0.35 | |
| 604 | 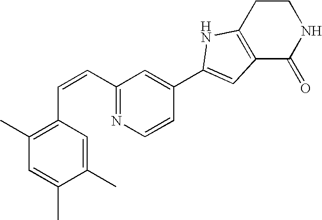 | 2-{2-[(Z)-2-(2,4,5-trimethylphenyl)vinyl]pyridin-4-yl}-1,5,6,7- tetrahydro-4H-pyrrolo[3,2-c]pyridin-4-one | 0.354 | |
| 605 | 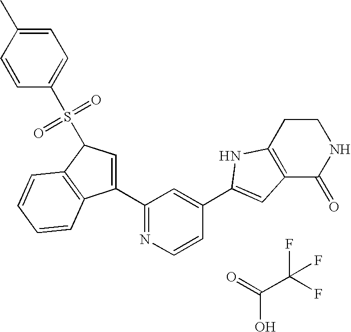 | 2-(2-{1-[(4-methylphenyl)sulfonyl]-1H-indol-3-yl}pyridin-4-yl)- 1,5,6,7-tetrahydro-4H-pyrrolo[3,2-c]pyridin-4-one trifluoroacetate | 0.359 | |
| 606 | 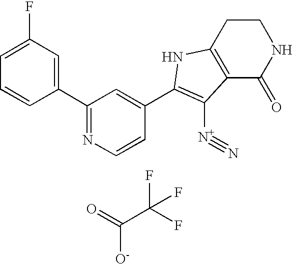 | 2-[2-(3-fluorophenyl)pyridin-4-yl]-4-oxo-4,5,6,7-tetrahydro-1H- pyrrolo[3,2-c]pyridine-3-diazonium trifluoroacetate | 0.359 | |
| 607 | 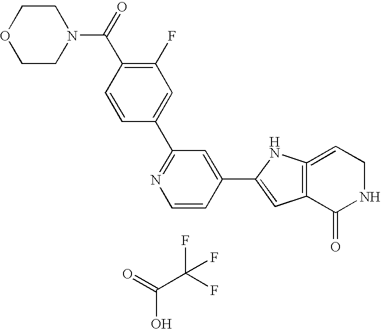 | 2-{2-[3-fluoro-4-(morpholin-4-ylcarbonyl)phenyl]pyridin-4-yl}-7- methyl-1,5,6,7-tetrahydro-4H-pyrrolo[3,2-c]pyridin-4-one trifluoroacetate | 0.36 | |
| 608 | 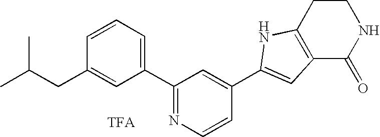 | 2-[2-(3-isobutylphenyl)pyridin-4-yl]-1,5,6,7-tetrahydro-4H- pyrrolo[3,2-c]pyridin-4-one trifluoroacetate | 0.363 | |
| 609 | 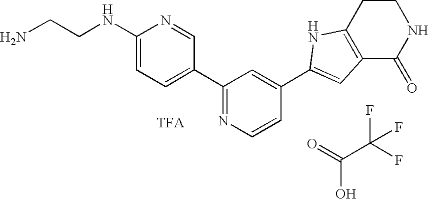 | 2-{6′-[(2-aminoethyl)amino]-2,3-bipyridin-4-yl}-1,5,6,7- tetrahydro-4H-pyrrolo[3,2-c]pyridin-4-one trifluoroacetate | | |
| 610 | 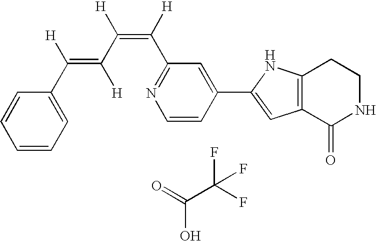 | 2-{2-[(1Z,3E)-4-phenylbuta-1,3-dienyl]pyridin-4-yl}-1,5,6,7- tetrahydro-4H-pyrrolo[3,2-c]pyridin-4-one trifluoroacetate | 0.365 | |
| 611 | 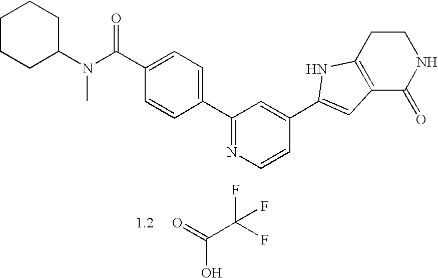 | N-cyclohexyl-N-methyl-4-[4-(4-oxo-4,5,6,7-tetrahydro-1H- pyrrolo[3,2-c]pyridin-2-yl)pyridin-2-yl]benzamide trifluoroacetate | 0.368 | |
| 612 | 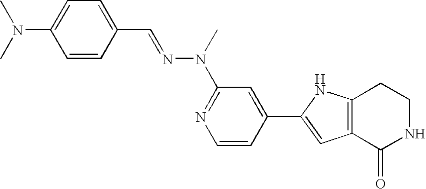 | 4-(dimethylamino)benzaldehyde methyl[4-(4-oxo-4,5,6,7- tetrahydro-1H-pyrrolo[3,2-c]pyridin-2-yl)pyridin-2-yl]hydrazone | 0.376 | |
| 613 | 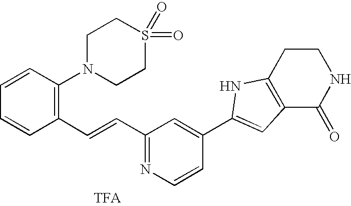 | 2-(2-{(E)-2-[2-(1,1-dioxidothiomorpholin-4-yl)phenyl]vinyl}pyridin- 4-yl)-1,5,6,7-tetrahydro-4H-pyrrolo[3,2-c]pyridin-4-one trifluoroacetate | 0.385 | |
| 614 | 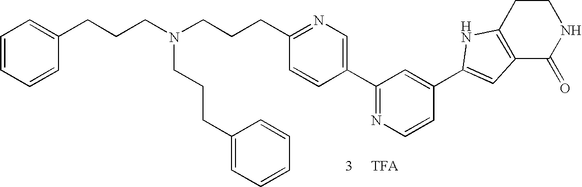 | 2-(6′-{3-[bis(3-phenylpropyl)amino]propyl}-2,3′-bipyridin-4-yl)- 1,5,6,7-tetrahydro-4H-pyrrolo[3,2-c]pyridin-4-one trifluoroacetate | 0.391 | |
| 615 | 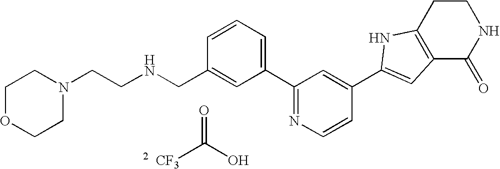 | 2-[2-(3-{[(2-morpholin-4-ylethyl)amino]methyl)phenyl)pyridin-4- yl]-1,5,6,7-tetrahydro-4H-pyrrolo[3,2-c]pyridin-4-one trifluoroacetate | 0.392 | |
| 616 | 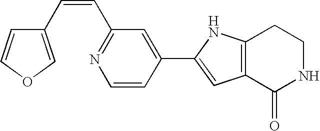 | 2-{2-[(Z)-2-(3-furyl)vinyl]pyridin-4-yl}-1,5,6,7-tetrahydro-4H- pyrrolo[3,2-c]pyridin-4-one | 0.398 | |
| 617 | 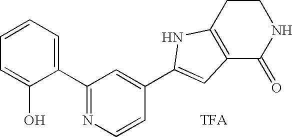 | 2-[2-(2-hydroxyphenyl)pyridin-4-yl]-1,5,6,7-tetrahydro-4H- pyrrolo[3,2-c]pyridin-4-one trifluoroacetate | 0.41 | |
| 618 | 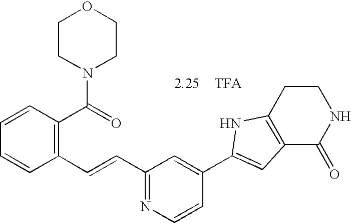 | 2-(2-{(E)-2-[2-(morpholin-4-ylcarbonyl)phenyl]vinyl}pyridin-4-yl)- 1,5,6,7-tetrahydro-4H-pyrrolo[3,2-c]pyridin-4-one trifluoroacetate | 0.416 | |
| 619 | 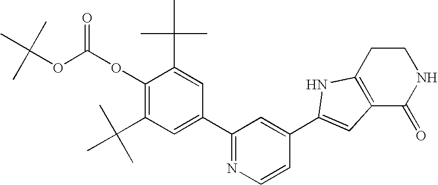 | tert-butyl 2,6-di-tert-butyl-4-[4-(4-oxo-4,5,6,7-tetrahydro-1H- pyrrolo[3,2-c]pyridin-2-yl)pyridin-2-yl]phenyl carbonate | 0.423 | |
| 620 | 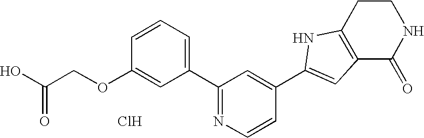 | {3-[4-(4-oxo-4,5,6,7-tetrahydro-1H-pyrrolo[3,2-c]pyridin-2-yl)pyridin-2-yl]phenoxy}acetic acid hydrochloride | 0.43 | |
| 621 | 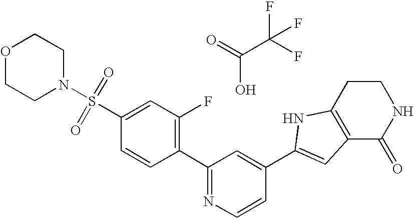 | 2-{2-[2-fluoro-4-(morpholin-4-ylsulfonyl)phenyl]pyridin-4-yl}- 1,5,6,7-tetrahydro-4H-pyrrolo[3,2-c]pyridin-4-one trifluoroacetate | 0.432 | |
| 622 | 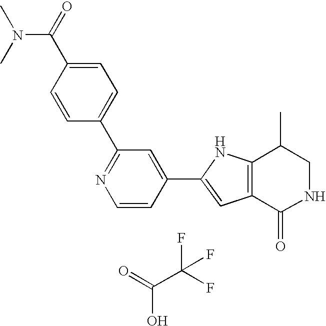 | N,N-dimethyl-4-[4-(7-methyl-4-oxo-4,5,6,7-tetrahydro-1H- pyrrolo[3,2-c]pyridin-2-yl)pyridin-2-yl]benzamide trifluoroacetate | 0.435 | |
| 623 | 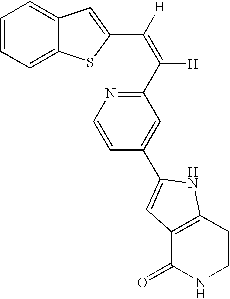 | 2-{2-[(Z)-2-(1-benzothien-2-yl)vinyl]pyridin-4-yl}-1,5,6,7- tetrahydro-4H-pyrrolo[3,2-c]pyridin-4-one | 0.442 | |
| 624 | 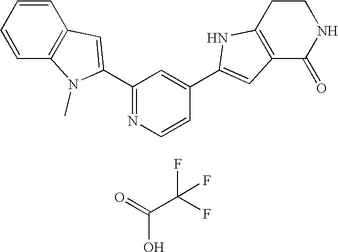 | 2-[2-(1-methyl-1H-indol-2-yl)pyridin-4-yl]-1,5,6,7-tetrahydro-4H- pyrrolo[3,2-c]pyridin-4-one trifluoroacetate | 0.444 | |
| 625 | 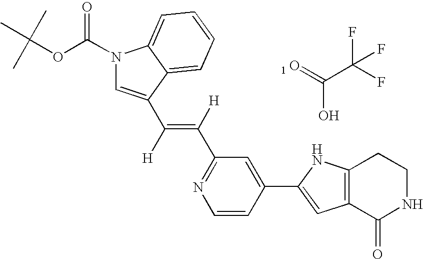 | tert-butyl 3-{(E)-2-[4-(4-oxo-4,5,6,7-tetrahydro-1H-pyrrolo[3,2- c]pyridin-2-yl)pyridin-2-yl]ethenyl}-1H-indole-1-carboxylate trifluoroacetate | 0.444 | |
| 626 | 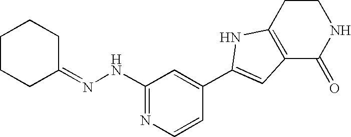 | 2-[2-(2-cyclohexylidenehydrazino)pyridin-4-yl]-1,5,6,7- tetrahydro-4H-pyrrolo[3,2-c]pyridin-4-one | 0.446 | |
| 627 | 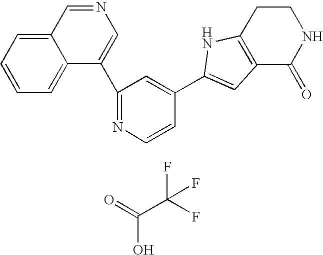 | 2-(2-isoquinolin-4-ylpyridin-4-yl)-1,5,6,7-tetrahydro-4H- pyrrolo[3,2-c]pyridin-4-one trifluoroacetate | 0.447 | |
| 628 | 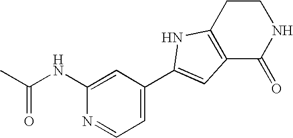 | N-[4-(4-oxo-4,5,6,7-tetrahydro-1H-pyrrolo[3,2-c]pyridin-2- yl)pyridin-2-yl]acetamide | 0.455 | |
| 629 | 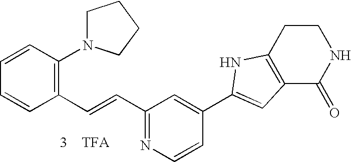 | 2-{2-[(E)-2-(2-pyrrolidin-1-ylphenyl)vinyl]pyridin-4-yl}-1,5,6,7- tetrahydro-4H-pyrrolo[3,2-c]pyridin-4-one trifluoroacetate | 0.458 | |
| 630 | 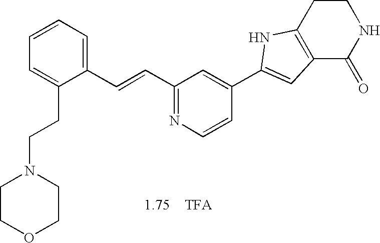 | 2-(2-{(E)-2-[2-(2-morpholin-4-ylethyl)phenyl]vinyl}pyridin-4-yl)- 1,5,6,7-tetrahydro-4H-pyrrolo[3,2-c]pyridin-4-one trifluoroacetate | 0.471 | |
| 631 | 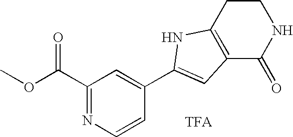 | methyl 4-(4-oxo-4,5,6,7-tetrahydro-1H-pyrrolo[3,2-c]pyridin-2- yl)pyridine-2-carboxylate trifluoroacetate | 0.479 | |
| 632 | 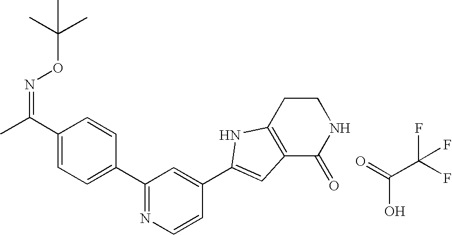 | 2-(2-{4-[(1Z)-N-(tert-butoxy)ethanimidoyl]phenyl}pyridin-4-yl)- 1,5,6,7-tetrahydro-4H-pyrrolo[3,2-c]pyridin-4-one trifluoroacetate | 0.488 | |
| 633 | 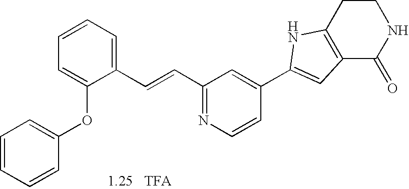 | 2-{2-[(E)-2-(2-phenoxyphenyl)vinyl]pyridin-4-yl{-1,5,6,7- tetrahydro-4H-pyrrolo[3,2-c]pyridin-4-one trifluoroacetate | 0.497 | |
| 634 | 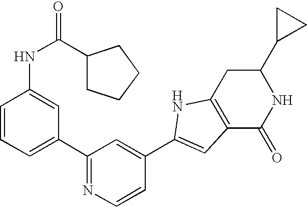 | N-{3-[4-(6-cyclopropyl-4-oxo-4,5,6,7-tetrahydro-1H-pyrrolo[3,2- c]pyridin-2-yl)pyridin-2-yl]phenyl}cyclopentanecarboxamide | 0.509 | |
| 635 | 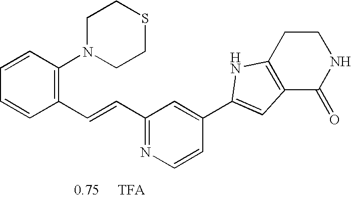 | 2-{2-[(E)-2-(2-thiomorpholin-4-ylphenyl)vinyl]pyridin-4-yl}- 1,5,6,7-tetrahydro-4H-pyrrolo[3,2-c]pyridin-4-one trifluoroacetate | 0.527 | |
| 636 | 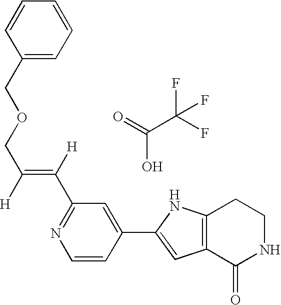 | 2-{2-[(1E)-3-(benzyloxy)prop-1-enyl]pyridin-4-yl}-1,5,6,7- tetrahydro-4H-pyrrolo[3,2-c]pyridin-4-one trifluoroacetate or 2- {2-[(1E)-3-(benzyloxy)prop-1-enyl]pyridin-4-yl}-1,5,6,7- tetrahydro-4H-pyrrolo[3,2-c]pyridin-4-one trifluoroacetate | 0.533 | |
| 637 | 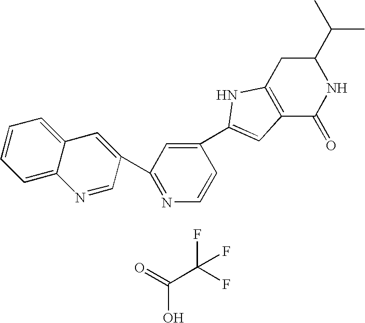 | 6-isopropyl-2-(2-quinolin-3-ylpyridin-4-yl)-1,5,6,7-tetrahydro-4H- pyrrolo[3,2-c]pyridin-4-one trifluoroacetate | 0.546 | |
| 638 | 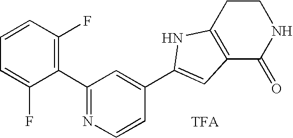 | 2-[2-(2,6-difluorophenyl)pyridin-4-yl]-1,5,6,7-tetrahydro-4H- pyrrolo[3,2-c]pyridin-4-one trifluoroacetate | 0.551 | |
| 639 | 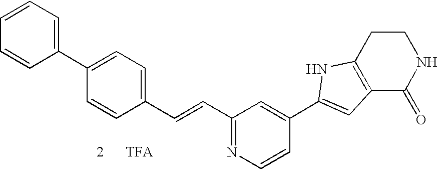 | 2-{2-[(E)-2-(1,1′-biphenyl-4-yl)vinyl]pyridin-4-yl}-1,5,6,7- tetrahydro-4H-pyrrolo[3,2-c]pyridin-4-one trifluoroacetate | 0.562 | |
| 640 | 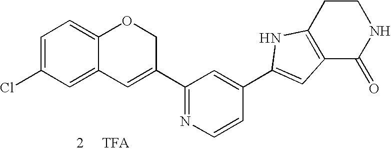 | 2-[2-(6-chloro-2H-chromen-3-yl)pyridin-4-yl]-1,5,6,7-tetrahydro- 4H-pyrrolo[3,2-c]pyridin-4-one trifluoroacetate | 0.564 | |
| 641 | 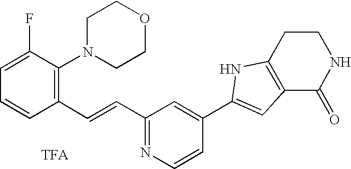 | 2-{2-[(E)-2-(3-fluoro-2-morpholin-4-ylphenyl)vinyl]pyridin-4-yl}- 1,5,6,7-tetrahydro-4H-pyrralo[3,2-c]pyridin-4-one trifluoroacetate | 0.58 | |
| 642 | 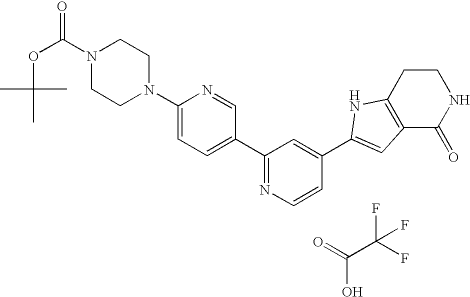 | tert-butyl 4-[4-(4-oxo-4,5,6,7-tetrahydro-1H-pyrrolo[3,2-c]pyridin- 2-yl)-2,3′-bipyridin-6′-yl]piperazine-1-carboxylate trifluoroacetate | 0.583 | |
| 643 | 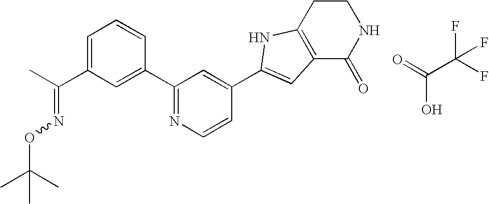 | 2-(2-{3-[(1E)-N-(tert-butoxy)ethanimidoyl]phenyl}pyridin-4-yl)- 1,5,6,7-tetrahydro-4H-pyrrolo[3,2-c]pyridin-4-one trifluoroacetate | 0.589 | |
| 644 | 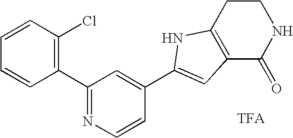 | 2-[2-(2-chlorophenyl)pyridin-4-yl]-1,5,6,7-tetrahydro-4H- pyrrolo[3,2-c]pyridin-4-one trifluoroacetate | 0.606 | |
| 645 | 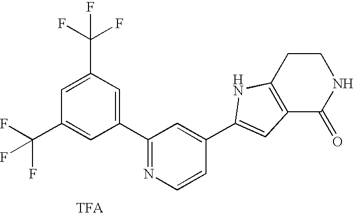 | 2-{2-[3,5-bis(trifluoromethyl)phenyl]pyridin-4-yl}-1,5,6,7- tetrahydro-4H-pyrrolo[3,2-c]pyridin-4-one trifluoroacetate | 0.613 | |
| 646 | 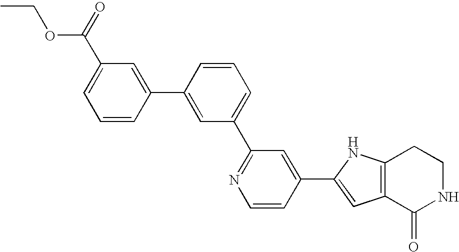 | ethyl 3′-[4-(4-oxo-4,5,6,7-tetrahydro-1H-pyrrolo[3,2-c]pyridin-2- yl)pyridin-2-yl]-1,1′-biphenyl-3-carboxylate | 0.622 | |
| 647 | 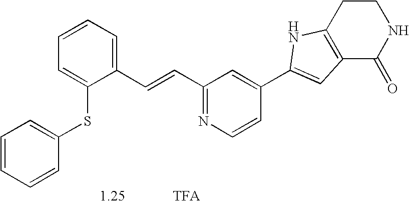 | 2-(2-{(E)-2-[2-(phenylthio)phenyl]vinyl}pyridin-4-yl)-1,5,6,7- tetrahydro-4H-pyrrolo[3,2-c]pyridin-4-one trifluoroacetate | 0.625 | |
| 648 | 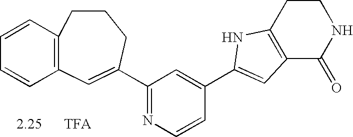 | 2-[2-(6,7-dihydro-5H-benzo[7]annulen-8-yl)pyridin-4-yl]-1,5,6,7- tetrahydro-4H-pyrrolo[3,2-c]pyridin-4-one trifluoroacetate | 0.643 | |
| 649 | 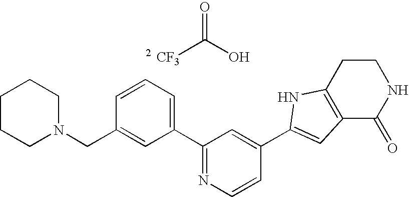 | 2-{2-[3-(pipendin-1-ylmethyl)phenyl]pyridin-4-yl}-1,5,6,7- tetrahydro-4H-pyrrolo[3,2-c]pyridin-4-one trifluoroacetate | 0.648 | |
| 650 | 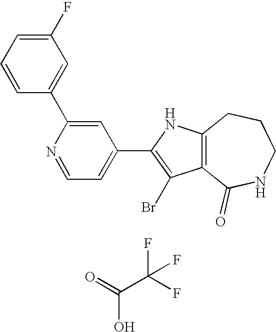 | 3-bromo-2-(2-(3-fluorophenyl)pyridin-4-yl]-5,6,7,8- tetrahydropyrrolo[3,2-c]azepin-4(1H)-one trifluoroacetate | 0.653 | |
| 651 | 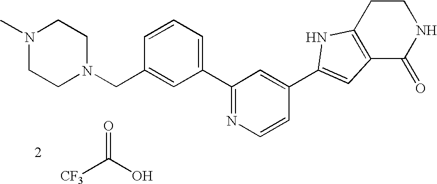 | 2-(2-{3-[(4-methylpiperazin-1-yl)methyl]phenyl}pyridin-4-yl)- 1,5,6,7-tetrahydro-4H-pyrrolo[3,2-c]pyridin-4-one trifluoroacetate | 0.664 | |
| 652 | 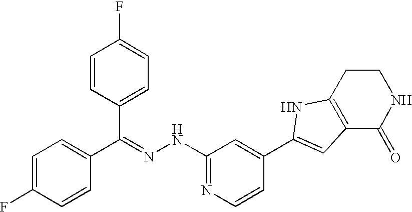 | 2-(2-{2-[bis(4-fluorophenyl)methylene]hydrazino}pyridin-4-yl)- 1,5,6,7-tetrahydro-4H-pyrrolo[3,2-c]pyridin-4-one | 0.666 | |
| 653 | 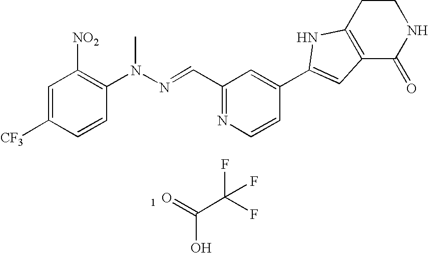 | 4-(4-oxo-4,5,6,7-tetrahydro-1H-pyrrolo[3,2-c]pyridin-2- yl)pyridine-2-carbaldehyde methyl[2-nitro-4- (trifluoromethyl)phenyl]hydrazone trifluoroacetate | 0.671 | |
| 654 | 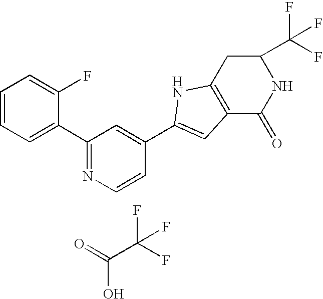 | 2-[2-(2-fluorophenyl)pyridin-4-yl]-6-(trifluoromethyl)-1,5,6,7- tetrahydro-4H-pyrrolo[3,2-c]pyridin-4-one trifluoroacetate | 0.703 | |
| 655 | 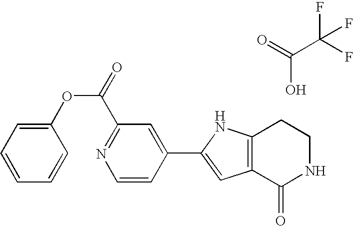 | phenyl 4-(4-oxo-4,5,6,7-tetrahydro-1H-pyrrolo[3,2-c]pyridin-2- yl)pyridine-2-carboxylate trifluoroacetate | 0.707 | |
| 656 | 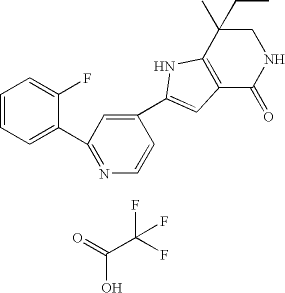 | 7-ethyl-2-[2-(2-fluorophenyl)pyridin-4-yl]-7-methyl-1,5,6,7- tetrahydro-4H-pyrrolo[3,2-c]pyridin-4-one trifluoroacetate | 0.708 | |
| 657 | 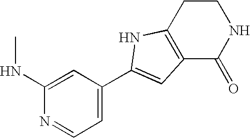 | 2-[2-(methylamino)pyridin-4-yl]-1,5,6,7-tetrahydro-4H- pyrrolo[3,2-c]pyridin-4-one | 0.715 | |
| 658 | 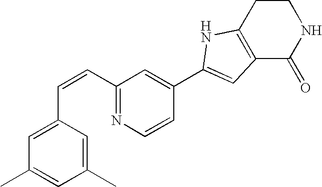 | 2-{2-[(Z)-2-(3,5-dimethylphenyl)vinyl]pyridin-4-yl}-1,5,6,7- tetrahydro-4H-pyrrolo[3,2-c]pyridin-4-one | 0.719 | |
| 659 | 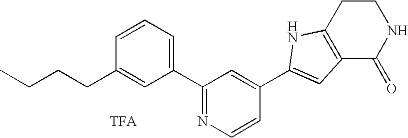 | 2-[2-(3-butylphenyl)pyridin-4-yl]-1,5,6,7-tetrahydro-4H- pyrrolo[3,2-c]pyridin-4-one trifluoroacetate | 0.721 | |
| 660 | 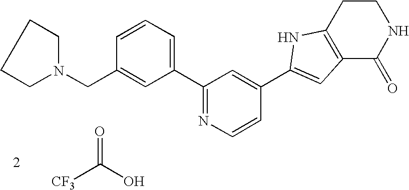 | 2-{2-[3-(pyrrolidin-1-ylmethyl)phenyl]pyridin-4-yl}-1,5,6,7- tetrahydro-4H-pyrrolo[3,2-c]pyridin-4-one trifluoroacetate | 0.75 | |
| 661 | 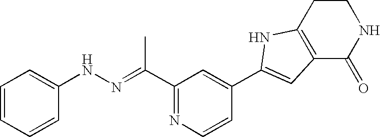 | 2-{2-[(1E)-N-phenylethanehydrazonoyl]pyridin-4-yl}-1,5,6,7- tetrahydro-4H-pyrrolo[3,2-c]pyridin-4-one | 0.763 | |
| 662 | 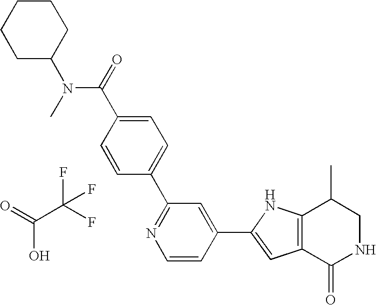 | N-cyclohexyl-N-methyl-4-[4-(7-methyl-4-oxo-4,5,6,7-tetrahydro- 1H-pyrrolo[3,2-c]pyridin-2-yl)pyridin-2-yl]benzamide trifluoroacetate | 0.769 | |
| 663 | 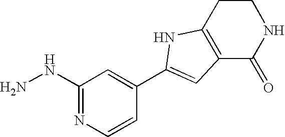 | 2-(2-hydrazinopyridin-4-yl)-1,5,6,7-tetrahydro-4H-pyrrolo[3,2- c]pyridin-4-one | 0.77 | |
| 664 | 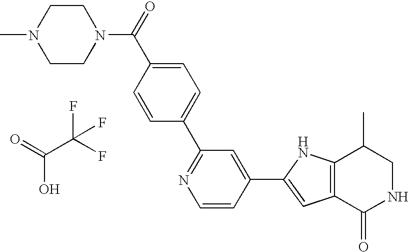 | 7-methyl-2-(2-{4-[(4-methylpiperazin-1- yl)carbonyl]phenyl}pyridin-4-yl)-1,5,6,7-tetrahydro-4H- pyrrolo[3,2-c]pyridin-4-one trifluoroacetate | 0.779 | |
| 665 | 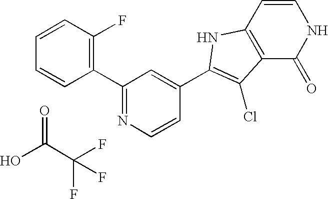 | 3-chloro-2-[2-(2-fluorophenyl)pyridin-4-yl]-1,5-dihydro-4H- pyrrolo[3,2-c]pyridin-4-one trifluoroacetate | 0.78 | |
| 666 | 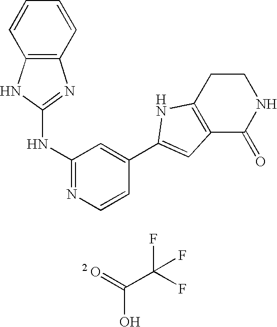 | 2-[2-(1H-benzimidazol-2-ylamino)pyridin-4-yl]-1,5,6,7-tetrahydro- 4H-pyrrolo[3,2-c]pyridin-4-one trifluoroacetate | 0.784 | |
| 667 | 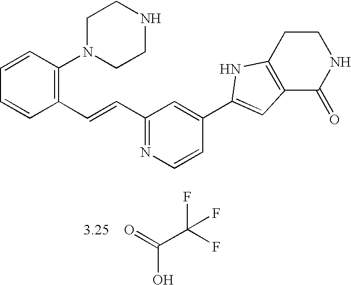 | 2-{2-[(E)-2-(2-piperazin-1-ylphenyl)vinyl]pyridin-4-yl}-1,5,6,7- tetrahydro-4H-pyrrolo[3,2-c]pyridin-4-one trifluoroacetate | 0.791 | |
| 668 | 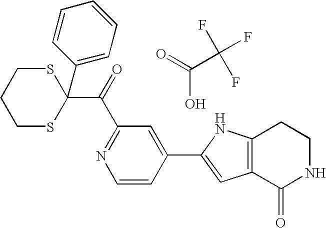 | 2-{2-[(2-phenyl-1,3-dithian-2-yl)carbonyl]pyridin-4-yl}-1,5,6,7- tetrahydro-4H-pyrrolo[3,2-c]pyridin-4-one trifluoroacetate | 0.794 | |
| 669 | 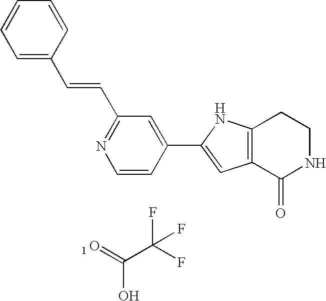 | 2-{2-[(E)-2-phenylethenyl]pyridin-4-yl}-1,5,6,7-tetrahydro-4H- imidazo[4,5-c]pyridin-4-one | 0.799 | |
| 670 | 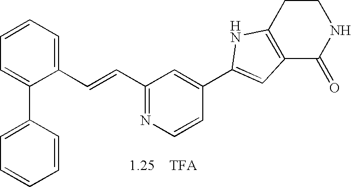 | 2-{2-[(E)-2-(1,1′-biphenyl-2-yl)vinyl]pyridin-4-yl}-1,5,6,7- tetrahydro-4H-pyrrolo[3,2-c]pyridin-4-one trifluoroacetate | 0.816 | |
| 671 | 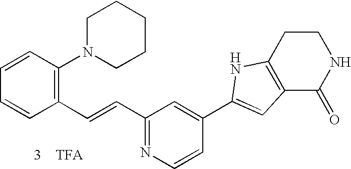 | 2-{2-[(E)-2-(2-piperidin-1-ylphenyl)vinyl]pyridin-4-yl}-1,5,6,7- tetrahydro-4H-pyrrolo[3,2-c]pyridin-4-one trifluoroacetate | 0.82 | |
| 672 | 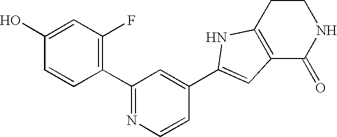 | 2-[2-(2-fluoro-4-hydroxyphenyl)pyridin-4-yl]-1,5,6,7-tetrahydro- 4H-pyrrolo[3,2-c]pyridin-4-one | 0.844 | |
| 673 | 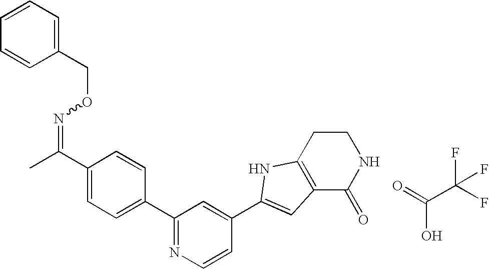 | 2-(2-{4-[(1Z)-N-(benzyloxy)ethanimidoyl]phenyl}pyridin-4-yl)- 1,5,6,7-tetrahydro-4H-pyrrolo[3,2-c]pyridin-4-one trifluoroacetate | 0.884 | |
| 674 | 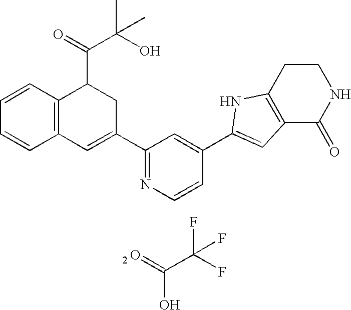 | 2-{2-[1-(2-hydroxy-2-methylpropanoyl)-1,2-dihydroquinolin-3- | yl]pyridin-4-yl}-1,5,6,7-tetrahydro-4H-pyrrolo[3,2-c]pyridin-4-one trifluoroacetate | 0.92 |
| 675 | 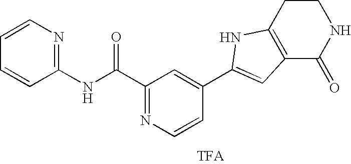 | 4-(4-oxo-4,5,6,7-tetrahydro-1H-pyrrolo[3,2-c]pyridin-2-yl)-N- pyridin-2-ylpyridine-2-carboxamide trifluoroacetate | 0.931 | |
| 676 | 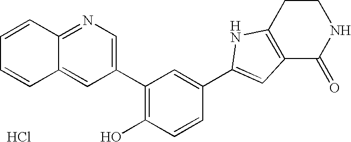 | 2-(4-hydroxy-3-quinolin-3-ylphenyl)-1,5,6,7-tetrahydro-4H- pyrrolo[3,2-c]pyridin-4-one hydrochloride | 0.937 | |
| 677 | 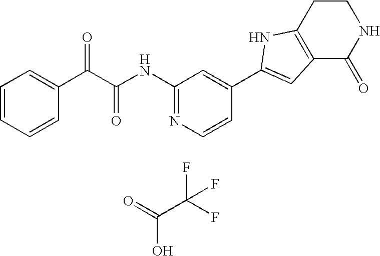 | 2-oxo-N-[4-(4-oxo-4,5,6,7-tetrahydro-1H-pyrrolo[3,2-c]pyridin-2-yl)pyridin-2-yl]-2-phenylacetamlde trifluoroacetate | 939 | |
| 678 | 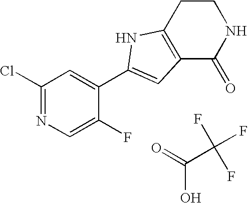 | 2-(2-chloro-5-fluoropyridin-4-yl)-1,5,6,7-tetrahydro-4H- pyrrolo[3,2-c]pyridin-4-one trifluoroacetate | 0.959 | |
| 679 | 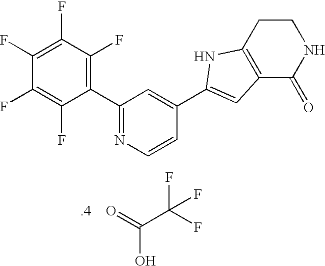 | 2-[2-(pentafluorophenyl)pyridin-4-yl]-1,5,6,7-tetrahydro-4H- pyrrolo[3,2-c]pyridin-4-one trifluoroacetate | 0.964 | |
| 680 | 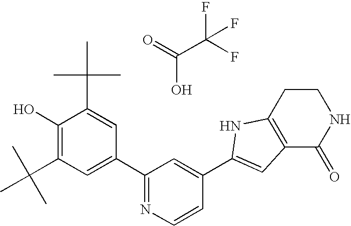 | 2-[2-(3,5-di-tert-butyl-4-hydroxyphenyl)pyridin-4-yl]-1,5,6,7- tetrahydro-4H-pyrrolo[3,2-c]pyridin-4-one trifluoroacetate | 0.976 | |
| 681 | 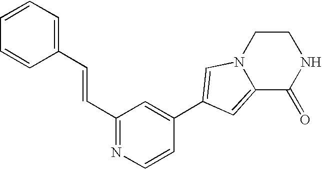 | 7-{2-[(E)-2-phenylethenyl]pyridin-4-yl}-3,4-dihydropyrrolo[1,2- a]pyrazln-1(2H)-one | 0.993 | |
| 682 | 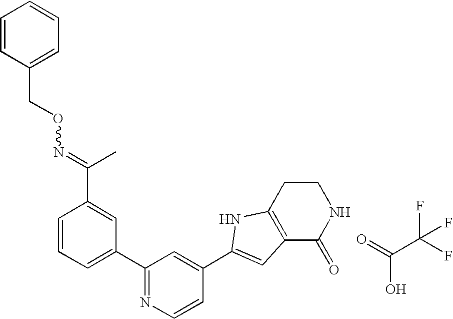 | 2-(2-{3-[(1E)-N-(benzyloxy)ethanimidoyl]phenyl}pyridin-4-yl)- 1,5,6,7-tetrahydro-4H-pyrrolo[3,2-c]pyridin-4-one trifluoroacetate | 1.01 | |
| 683 | 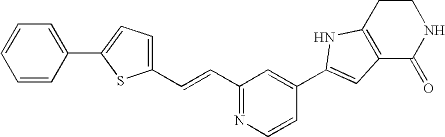 | 2-{2-[(E)-2-(5-phenylthien-2-yl)vinyl]pyridin-4-yl}-1,5,6,7- tetrahydro-4H-pyrrolo[3,2-c]pyridin-4-one | 1.02 | |
| 684 | 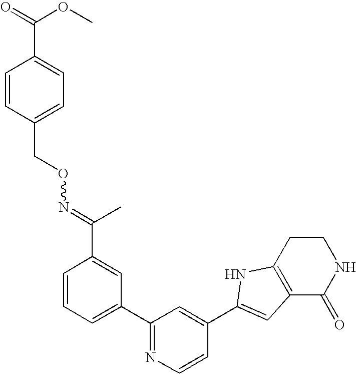 | methyl 4-({[((1E)-1-{3-[4-(4-oxo-4,5,6,7-tetrahydro-1H- pyrrolo[3,2-c]pyridin-2-yl)pyridin-2- yl]phenyl}ethylidene)amino]oxy}methyl)benzoate | 1.05 | |
| 685 | 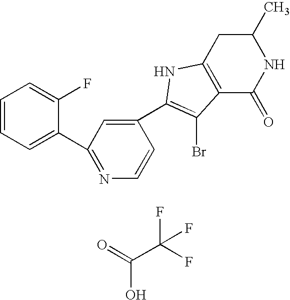 | 3-bromo-2-[2-(2-fluorophenyl)pyridin-4-yl]-6-methyl-1,5,6,7- tetrahydro-4H-pyrrolo[3,2-c]pyridin-4-pne trifluoroacetate | 1.06 | |
| 686 | 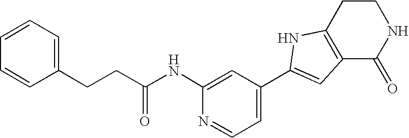 | N-[4-(4-oxo-4,5,6,7-tetrahydro-1H-pyrrolo[3,2-c]pyridin-2- yl)pyridin-2-yl]-3-phenylpropanamide | 1.06 | |
| 687 | 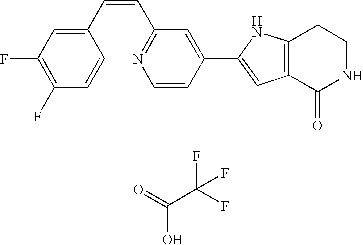 | 2-{2-[(Z)-2-(3,4-difluorophenyl)ethenyl]pyridin-4-yl}-1,5,6,7- tetrahydro-4H-pyrrolo[3,2-c]pyridin-4-one trifluoroacetate | 1.08 | |
| 688 | 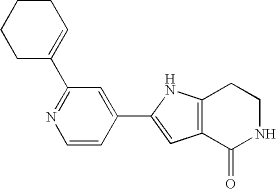 | 2-(2-cyclohex-1-en-1-ylpyridin-4-yl)-1,5,6,7-tetrahydro-4H- pyrrolo[3,2-c]pyridin-4-one | 1.09 | |
| 689 | 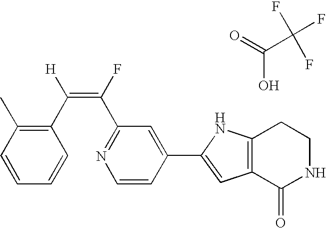 | 2-{2-[(E)-1-fluoro-2-(2-methylphenyl)vinyl]pyridin-4-yl}-1,5,6,7- tetrahydro-4H-pyrrolo[3,2-c]pyridin-4-one trifluoroacetate | 1.1 | |
| 690 | 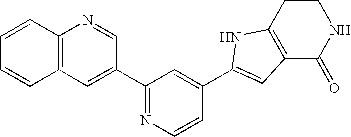 | 2-(2-quinolin-3-ylpyridin-4-yl)-1,5,6,7-tetrahydro-4H-imidazo[4,5- c]pyridin-4-one | 1.12 | |
| 691 | 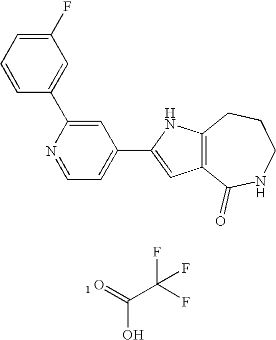 | 2-[2-(3-fluorophenyl)pyridin-4-yl]-5,6,7,8-tetrahydropyrrolo[3,2- c]azepin-4(1H)-one trifluoroacetate | 1.14 | |
| 692 | 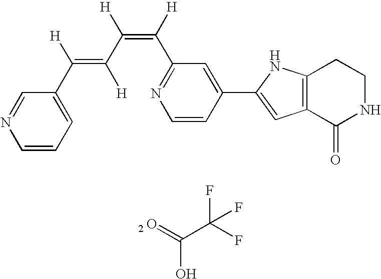 | 2-{2-[(1Z,3E)-4-pyridin-3-ylbuta-1,3-dienyl]pyridin-4-yl}-1,5,6,7- tetrahydro-4H-pyrrolo[3,2-c]pyridin-4-one trifluoroacetate | 1.14 | |
| 693 | 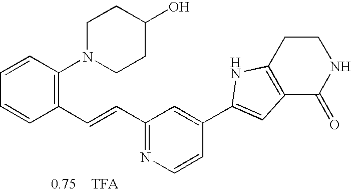 | 2-(2-{(E)-2-[2-(4-hydroxpiperidin-1-yl)phenyl]vinyl}pyridin-4-yl)- 1,5,67-tetrahydro-4H-pyrrolo[3,2-c]pyridin-4-one trifluoroacetate | 1.14 | |
| 694 | 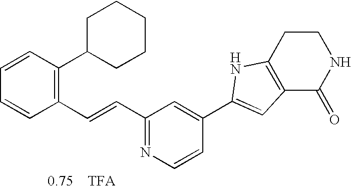 | 2-{2-[(E)-2-(2-cyclohexylphenyl)vinyl]pyridin-4-yl}-1,5,6,7- tetrahydro-4H-pyrrolo[3,2-c]pyridin-4-one trifluoroacetate | 1.15 | |
| 695 | 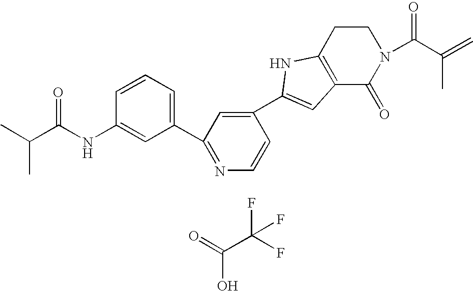 | N-{3-[4-(5-methacryloyl-4-oxo-4,5,6,7-tetrahydro-1H-pyrrolo[3,2- c]pyridin-2-yl)pyridin-2-yl]phenyl}-2-methylacrylamide trifluoroacetate | 1.19 | |
| 696 | 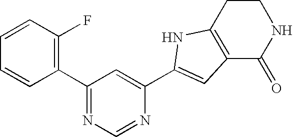 | 2-[6-(2-fluorophenyl)pyrimidin-4-yl]-1,5,6,7-tetrahydro-4H- pyrrolo[3,2-c]pyridin-4-one | 1.19 | |
| 697 | 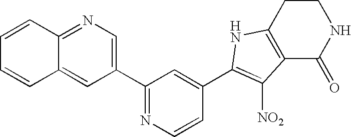 | 3-nitro-2-(2-quinolin-3-ylpyridin-4-yl)-1,5,6,7-tetrahydro-4H- pyrrolo[3,2-c]pyridin-4-one | 1.22 | |
| 698 | 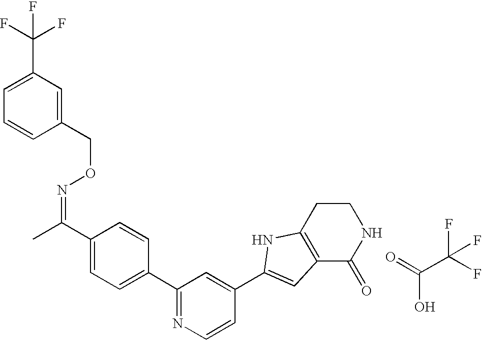 | 2-{2-[4-((1Z)-N-{[3- (trifluoromethyl)benzyl]oxy}ethanimidoyl)phenyl]pyridin-4-yl)- 1,5,6,7-tetrahydro-4H-pyrrolo[3,2-c]pyridin-4-one trifluoroacetate | 1.22 | |
| 699 | 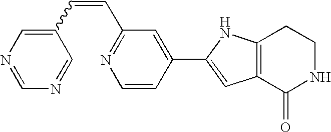 | 2[f2-[(Z)-2-pyrimdin-5-ylvinyl]pyridin-4-yl}-1,5,6,7-tetrahydro-4H- pyrrolo[3,2-c]pyridin-4-one | 1.24 | |
| 700 | 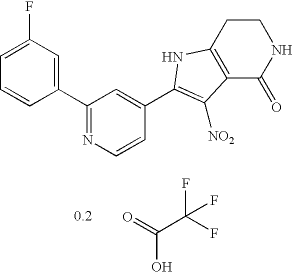 | 2-[2-(3-fluorophenyl)pyridin-4-yl]-3-nitro-1,5,6,7-tetrahydro-4H- pyrrolo[3,2-c]pyridin-4-one trifluoroacetate | 1.25 | |
| 701 | 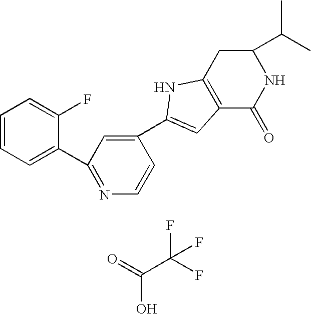 | 2-[2-(2-fluorophenyl)pyridin-4-yl]-6-isopropyl-1,5,6,7-tetrahydro- 4H-pyrrolo[3,2-c]pyridin-4-one triftuoroacetate | 1.29 | |
| 702 | 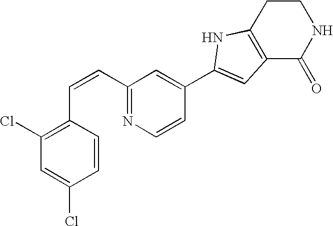 | 2-{2-[(Z)-2-(2,4-dichlorophenyl)vinyl]pyridin-4-yl}-1,5,6,7- tetrahydro-4H-pyrrolo[3,2-c]pyridin-4-one | 1.31 | |
| 703 | 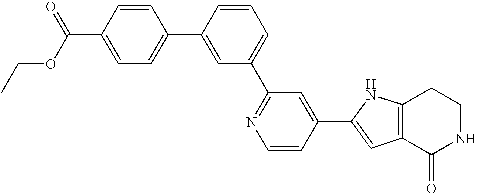 | ethyl 3′-[4-(4-oxo-4,5,6,7-tetrahydro-1H-pyrrolo[3,2-c]pyridin-2- yl)pyridin-2-yl]-1,1′-biphenyl-4-carboxylate | 1.32 | |
| 704 | 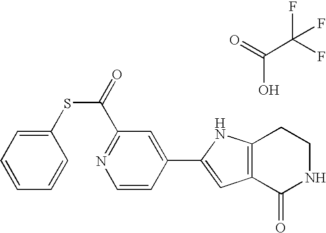 | S-phenyl 4-(4-oxo-4,5,6,7-tetrahydro-1H-pyrrolo[3,2-c]pyridin-2- yl)pyridine-2-carbothioate trifluoroacetate | 1.32 | |
| 705 | 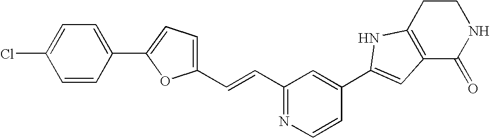 | 2-(2-{(E)-2-[5-(4-chlorophenyl)-2-furyl]vinyl}pyridin-4-yl)-1,5,6,7- tetrahydro-4H-pyrrolo[3,2-c]pyridin-4-one | 1.34 | |
| 706 | 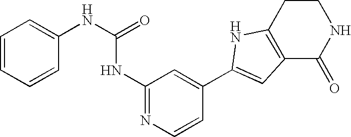 | N-[4-(4-oxo-4,5,6,7-tetrahydro-1H-pyrrolo[3,2-c]pyridin-2- yl)pyridin-2-yl]-N′-phenylurea | 1.34 | |
| 707 | 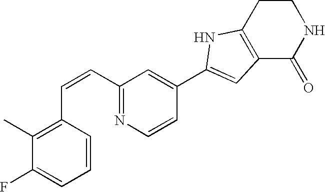 | 2-{2-[(Z)-2-(3-fluoro-2-methylphenyl)vinyl]pyridin-4-yl)-1,5,6,7- tetrahydro-4H-pyrrolo[3,2-c]pyridin-4-one | 1.4 | |
| 708 | 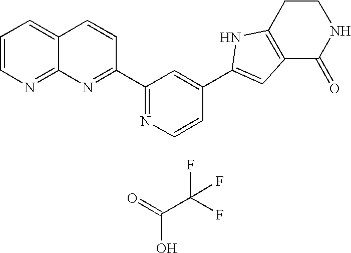 | 2-[3-(1,8-naphthyridin-2-yl)phenyl]-1,5,6,7-tetrahydro-4H- pyrrolo[3,2-c]pyridin-4-one trifluoroacetate | 1.45 | |
| 709 | 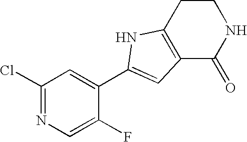 | 2-(2-chloro-5-fluoropyridin-4-yl)-1,5,6,7-tetrahydro-4H- pyrrolo[3,2-c]pyridin-4-one | 1.46 | |
| 710 | 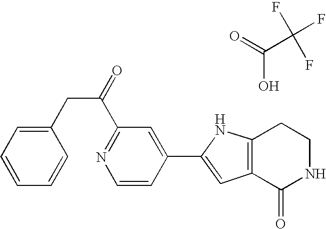 | 2-[2-(phenylacetyl)pyridin-4-yl]-1,5,6,7-tetrahydro-4H- pyrrolo[3,2-c]pyridin-4-one trifluoroacetate | 1.63 | |
| 711 | 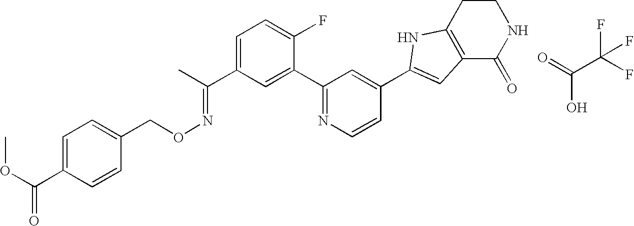 | methyl 4-({[((1E)-1-(4-fluoro-3-[4-(4-oxo-4,5,6,7-tetrahydro-1H- pyrrolo[3,2-c]pyridin-2-yl)pyridin-2- yl]phenyl}ethylidene)amino]oxy}methyl)benzoate trifluoroacetate | 1.64 | |
| 712 | 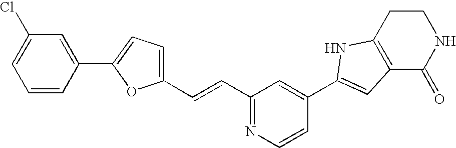 | 2-(2-{(E)-2-(5-(3-chlorophenyl)-2-furyl]vinyl}pyridin-4-yl)-1,5,6,7- tetrahydro-4H-pyrralo[3,2-c]pyridin-4-one | 1.7 | |
| 713 | 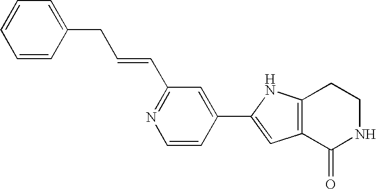 | 2-{2-[(1E)-3-phenylprop-1-enyl]pyridin-4-yl}-1,5,6,7-tetrahydro- 4H-pyrrolo[3,2-c]pyridin-4-one | 1.76 | |
| 714 | 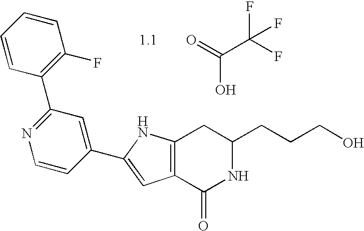 | 2-[2-(2-fluorophenyl)pyridin-4-yl]-6-(3-hydroxypropyl)-1,5,6,7- tetrahydro-4H-pyrrolo[3,2-c]pyridin-4-one trifluoroacetate | 1.8 | |
| 715 | 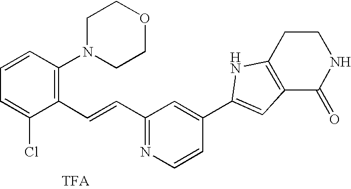 | 2-{2-[(E)-2-(2-chloro-6-morpholin-4-ylphenyl)vinyl]pyridin-4-yl}- 1,5,6,7-tetrahydro-4H-pyrrolo[3,2-c]pyridin-4-one trifluoroacetate | 1.8 | |
| 716 | 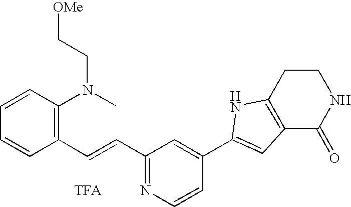 | 2-[2-((E)-2-{2-[(2- methoxyethyl)(methyl)amino]phenyl}vinyl)pyridin-4-yl]-1,5,6,7- tetrahydro-4H-pyrrolo[3,2-c]pyridin-4-one trifiuoroacetate | 1.81 | |
| 717 | 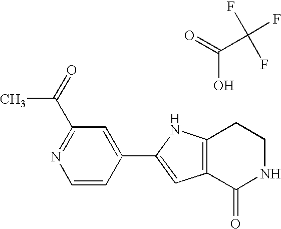 | 2-(2-acetylpyridin-4-yl)-1,5,6,7-tetrahydro-4H-pyrrolo[3,2- c]pyridin-4-one trifluoroacetate | 1.83 | |
| 718 | 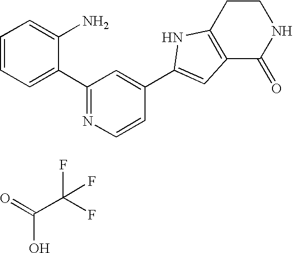 | 2-[2-(2-aminophenyl)pyridin-4-yl]-1,5,6,7-tetrahydro-4H- pyrrolo[3,2-c]pyridin-4-one trifluoroacetate | 1.85 | |
| 719 | 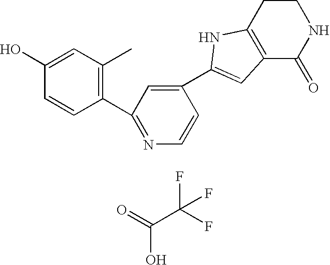 | 2-[2-(4-hydroxy-2-methylphenyl)pyridin-4-yl]-1,5,6,7-tetrahydro- 4H-pyrrolo[3,2-c]pyridin-4-one trifluoroacetate | 1.85 | |
| 720 | 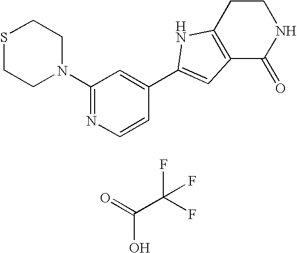 | 2-(2-thiomorpholin-4-ylpyridin-4-yl)-1,5,6,7-tetrahydro-4H- pyrrolo[3,2-c]pyridin-4-one trifluoroacetate | 1.85 | |
| 721 | 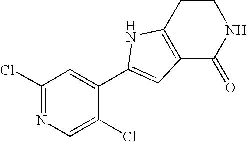 | 2-(2,5-dichloropyridin-4-yl)-1,5,6,7-tetrahydro-4H-pyrrolo[3,2- c]pyridin-4-one | 1.88 | |
| 722 | 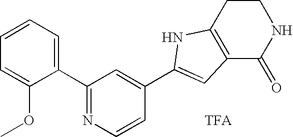 | 2-[2-(2-methoxyphenyl)pyridin-4-yl]-1,5,6,7-tetrahydro-4H- pyrrolo[3,2-c]pyridin-4-one trifluoroacetate | 2.02 | |
| 723 | 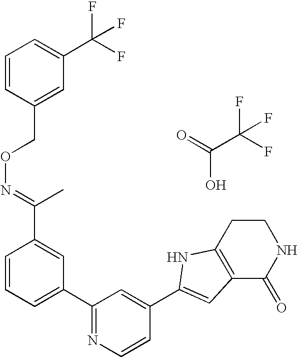 | 2-{2-[3-((1E)-N-{[3- (trifluoromethyl)benzl]ox}ethanimidoyl)phenyl]pyridin-4-yl}- 1,5,6,7-tetrahydro-4H-pyrrolo[3,2-c]pyridin-4-one trifluoroacetate | 2.06 | |
| 724 | 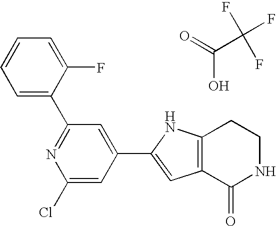 | 2-[2-chloro-6-(2-fluorophenyl)pyridin-4-yl]-1,5,6,7-tetrahydro-4H- pyrrolo[3,2-c]pyridin-4-one trifluoroacetate | 2.06 | |
| 725 | 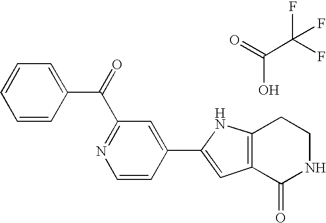 | 2-(2-benzoylpyridin-4-yl)-1,5,6,7-tetrahydro-4H-pyrrolo[3,2- c]pyridin-4-one trifluoroacetate | 2.09 | |
| 726 | 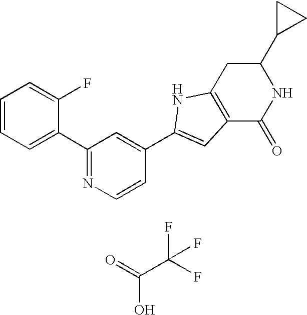 | 6-cyclopropyl-2-[2-(2-fluorophenyl)pyridin-4-yl]-1,5,6,7- tetrahydro-4H-pyrrolo[3,2-c]pyridin-4-one trifluoroacetate | 2.11 | |
| 727 | 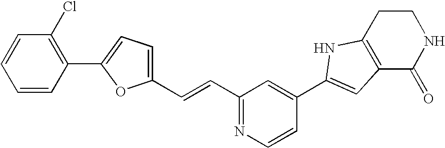 | 2-(2-{(E)-2-[5-(2-chlorophenyl)-2-furyl]vinyl}pyridin-4-yl)-1,5,6,7- tetrahydro-4H-pyrroto[3,2-c]pyridin-4-one | 2.18 | |
| 728 | 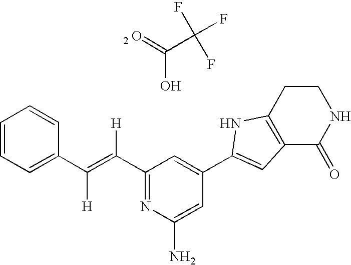 | 2-{2-amino-6-[(E)-2-phenylvinyl]pyridin-4-yl)-1,5,6,7-tetrahydro- 4H-pyrrolo[3,2-c]pyridin-4-one trifluoroacetate | 2.21 | |
| 729 | 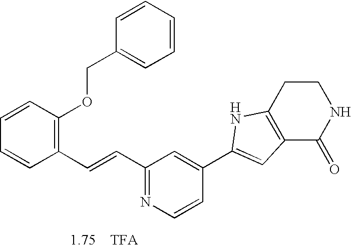 | 2-(2-{(E)-2-[2-(benzyloxy)phenyl]vinyl}pyridin-4-yl)-1,5,6,7- tetrahydro-4H-pyrrolo[3,2-c]pyridin-4-one trifluoroacetate | 2.21 | |
| 730 | 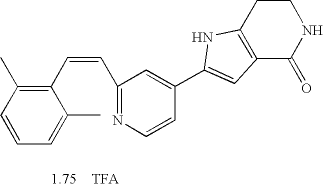 | 2-{2-[(Z)-2-(2,6-dimethylphenyl)vinyl]pyridin-4-yl}-1,5,6,7- tetrahydro-4H-pyrrolo[3,2-c]pyridin-4-one trifluoroacetate | 2.24 | |
| 731 | 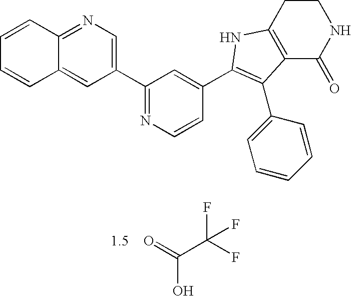 | 3-phenyl-2-(2-quinolin-3-ylpyridin-4-yl)-1,5,6,7-tetrahydro-4H- pyrrolo[3,2-c]pyridin-4-one trifluoroacetate | 2.26 | |
| 732 | 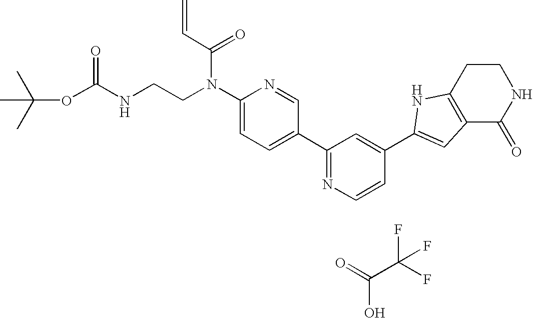 | tert-butyl 2-{acryloyl[4-(4-oxo-4,5,6,7-tetrahydro-1H-pyrrolo[3,2- c]pyridin-2-yl)-2,3-bipyridin-6′-yl]amino}ethylcarbamate trifluoroacetate | 2.33 | |
| 733 | 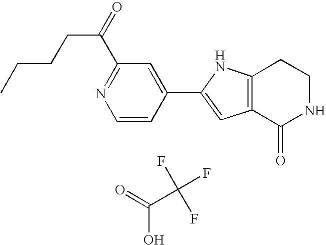 | 2-(2-pentanoylpyridin-4-yl)-1,5,6,7-tetrahydro-4H-pyrrolo[3,2- c]pyridin-4-one trifluoroacetate | 2.41 | |
| 734 | 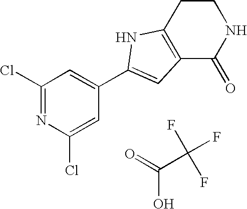 | 2-(2,6-dichloropyridin-4-yl)-1,5,6,7-tetrahydro-4H-pyrrolo[3,2- c]pyridin-4-one trifluoroacetate | 2.42 | |
| 735 | 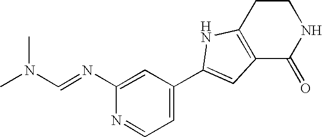 | N,N-dimethyl-N′-[4-(4-oxo-4,5,6,7-tetrahydro-1H-pyrrolo[3,2- c]pyridin-2-yl)pyridin-2-yl]imidoformamide | 2.45 | |
| 736 | 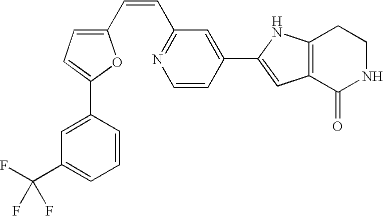 | 2-[2-((Z)-2-{5-[3-(trifluoromethyl)phenyl]-2-furyl}vinyl)pyridin-4- yl]-1,5,6,7-tetrahydro-4H-pyrrolo[3,2-c]pyridin-4-one | 2.5 | |
| 737 | 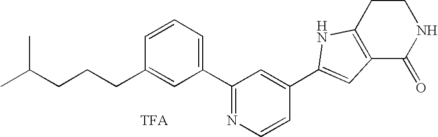 | 2-{2-[3-(4-methylpentyl)phenyl]pyridin-4-yl}-1,5,6,7-tetrahydro- 4H-pyrrolo[3,2-c]pyridin-4-one trifluoroacetate | 2.55 | |
| 738 | 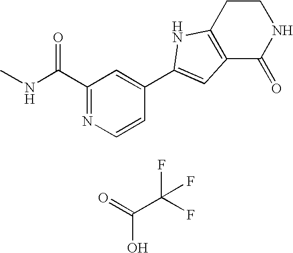 | N-methyl-4-(4-oxo-4,5,6,7-tetrahydro-1H-pyrrolo[3,2-c]pyridin-2- yl)pyridine-2-carboxamide trifluoroacetate | 2.62 | |
| 739 | 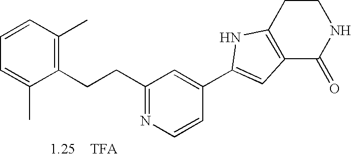 | 2-{2-[2-(2,6-dimethylphenyl)ethyl]pyridin-4-yl}-1,5,6,7-tetrahydro- 4H-pyrrolo[3,2-c]pyridin-4-one trifluoroacetate | 2.62 | |
| 740 | 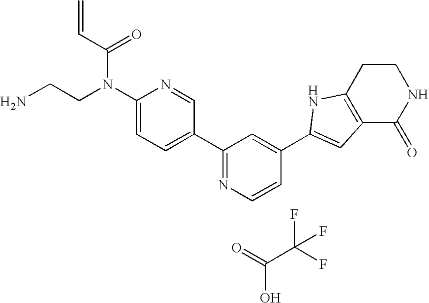 | N-(2-aminoethyl)-N-[4-(4-oxo-4,5,6,7-tetrahydro-1H-pyrrolo[3,2- c]pyridin-2-yl)-2,3′-bipyridin-6′-yl]acrylamide trifluoroacetate | 2.69 | |
| 741 | 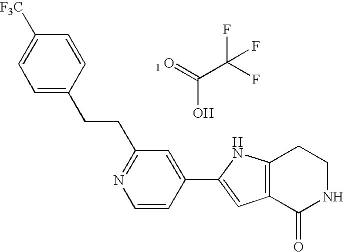 | 2-(2-{2-[4-(trifluoromethyl)phenyl]ethyl}pyridin-4-yl)-1,5,6,7- tetrahydro-4H-pyrrolo[3,2-c]pyridin-4-one trifluoroacetate | 2.82 | |
| 742 | 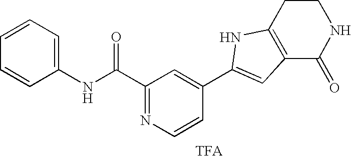 | 4-(4-oxo-4,5,6,7-tetrahydro-1H-pyrrolo[3,2-c]pyridin-2-yl)-N- phenylpyridine-2-carboxamide trifluoroacetate | 2.86 | |
| 743 | 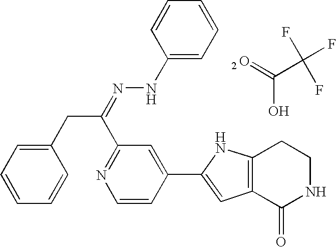 | 2-{2-[(1Z)-N,2-diphenylethanehydrazonoyl]pyridin-4-yl}-1,5,6,7- tetrahydro-4H-pyrrolo[3,2-c]pyridin-4-one trifluoroacetate | 2.97 | |
| 744 | 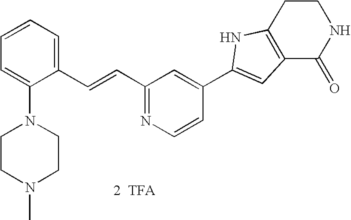 | 2-(2-{(E)-2-[2-(4-methylpiperazin-1-yl)phenyl]vinyl}pyridin-4-yl)- 1,5,6,7-tetrahydro-4H-pyrrolo[3,2-c]pyridin-4-one trifluoroacetate | 2.99 | |
| 745 | 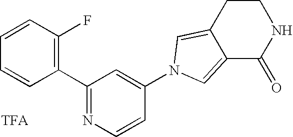 | 2-[2-(2-fluorophenyl)pyridin-4-yl]-2,5,6,7-tetrahydro-4H- pyrrolo[3,4-c]pyridin-4-one trifluoroacetate | 3.18 | |
| 746 | 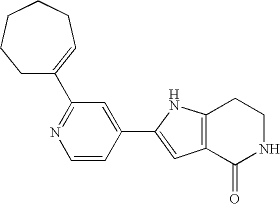 | 2-(2-cyclohept-1-en-1-ylpyridin-4-yl)-1,5,6,7-tetrahydro-4H- pyrrolo[3,2-c]pyridin-4-one | 3.21 | |
| 747 | 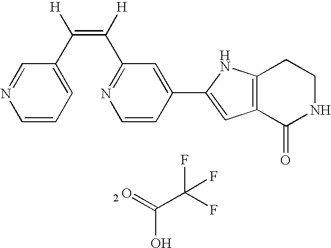 | 2-{2-[(Z)-2-pyridin-3-ylethenyl]pyridin-4-yl}-1,5,6,7-tetrahydro- 4H-pyrrolo[3,2-c]pyridin-4-one trifluoroacetate | 3.33 | |
| 748 | 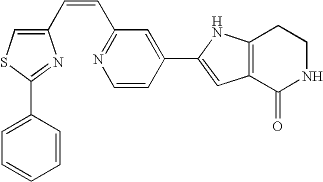 | 2-{2-[(Z)-2-(2-phenyl-1,3-thiazol-4-yl)vinyl]pyridin-4-yl)-1,5,6,7- tetrahydro-4H-pyrrolo[3,2-c]pyridin-4-one | 3.62 | |
| 749 | 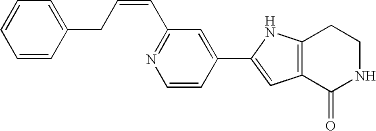 | 2-{2-[(1Z)-3-phenylprop-1-enyl]pyridin-4-yl}-1,5,6,7-tetrahydro- 4H-pyrrolo[3,2-c]pyridin-4-one | 3.75 | |
| 750 | 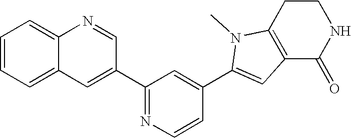 | 1-methyl-2-(2-quinolin-3-ylpyridin-4-yl)-1,5,6,7-tetrahydro-4H- pyrrolo[3,2-c]pyridin-4-one | 4.13 | |
| 751 | 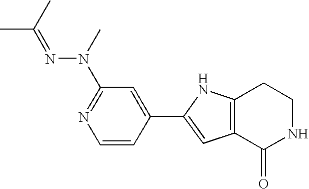 | 2-55 2-[1-methyl-2-(1-methylethylidene)hydrazino]pyridin-4-yl}- 1,5,6,7-tetrahydro-4H-pyrrolo[3,2-c]pyridin-4-one | 4.22 | |
| 752 | 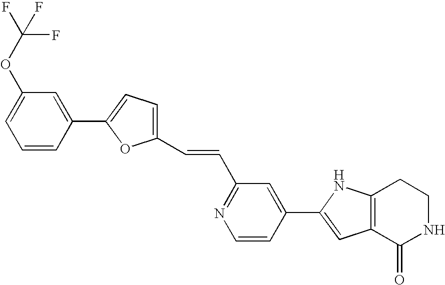 | 2-[2-((E)-2-{5-[3-(trifluoromethoxy)phenyl]-2-furyl)vinyl)pyridin-4- yl]-1,5,6,7-tetrahydro-4H-pyrrolo[3,2-c]pyridin-4-one | 4.26 | |
| 753 | 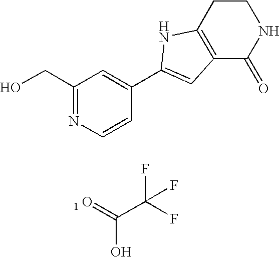 | 2-[2-(hydroxymethyl)pyridin-4-yl]-1,5,6,7-tetrahydro-4H- pyrrolo[3,2-c]pyridin-4-one trifluoroacetate | 4.39 | |
| 754 | 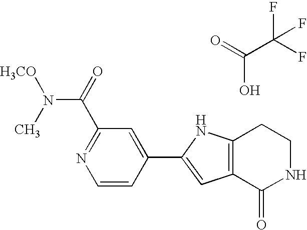 | N-methoxy-N-methyl-4-(4-oxo-4,5,6,7-tetrahydro-1H-pyrrolo[3,2- c]pyridin-2-yl)pyridine-2-carboxamide trifluoroacetate | 4.47 | |
| 755 | 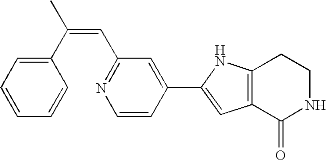 | 2-{2-[(1Z)-2-phenylprop-1-enyl]pyridin-4-yl)-1,5,6,7-tetrahydro- 4H-pyrrolo[3,2-c]pyridin-4-one | 4.82 | |
| 756 | 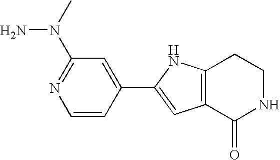 | 2-[2-(1-methylhydrazino)pyridin-4-yl]-1,5,6,7-tetrahydro-4H- pyrrolo[3,2-c]pyridin-4-one | 4.91 | |
| 757 | 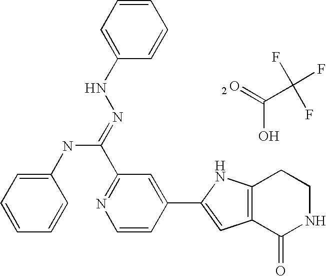 | 2-{2-[(1E)-N,2-diphenylethanehydrazonoyl]pyridin-4-yl}-1,5,6,7- tetrahydro-4H-pyrrolo[3,2-c]pyridin-4-one trifluroacetate | 515 | |
| 758 | 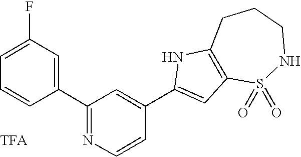 | 7-[2-(3-fluorophenyl)pyridin-4-yl]-3,4,5,6-tetrahydro-2H- pyrrolo[2,3-f][1,2]thiazepine 1,1-dioxide trifluoroacetate | 5.2 | |
| 759 | 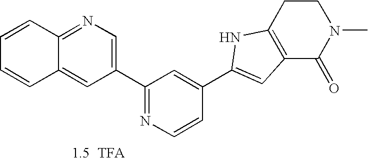 | 5-methyl-2-(2-quinolin-3-ylpyridin-4-yl)-1,5,6,7-tetrahydro-4H- pyrrolo[3,2-c]pyridin-4-one trifluoroacetate | 5.53 | |
| 760 | 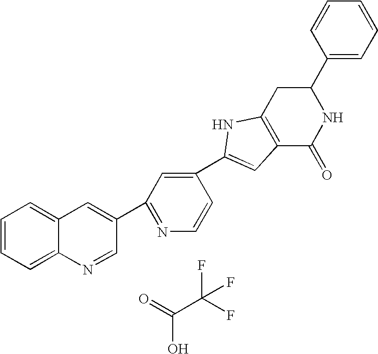 | 6-phenyl-2-(2-quinolin-3-ylpyridin-4-yl)-1,5,6,7-tetrahydro-4H- pyrrolo[3,2-c]pyridin-4-one trifluoroacetate | 5.67 | |
| 761 | 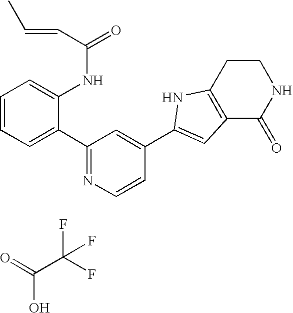 | (2E)-N-{2-[4-(4-oxo-4,5,6,7-tetrahydro-1H-pyrrolo[3,2-c]pyridin- 2-yl)pyridin-2-yl]phenyl}but-2-enamide trifluoroacetate | 5.73 | |
| 762 | 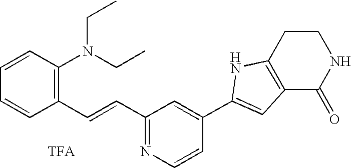 | 2-(2-{(E)-2-(2-(diethylamino)phenyl]vinyl}pyridin-4-yl)-1,5,6,7- tetrahydro-4H-pyrrolo[3,2-c]pyridin-4-one trifluoroacetate | 5.77 | |
| 763 | 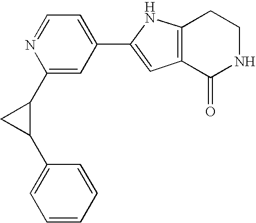 | 2-[2-(2-phenylcyclopropyl)pyridin-4-yl]-1,5,6,7-tetrahydro-4H- pyrrolo[3,2-c]pyridin-4-one | 6.24 | |
| 764 | 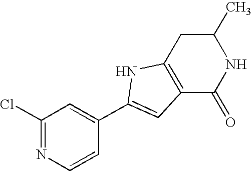 | 2-(2-chloropyridin-4-yl)-6-methyl-1,5,6,7-tetrahydro-4H- pyrrolo[3,2-c]pyridin-4-one | 6.39 | |
| 765 | 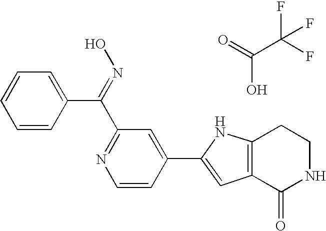 | 2-{2-[(E)-(hydroxyimino)(phenyl)methyl]pyridin-4-yl}-1,5,6,7- tetrahydro-4H-pyrrolo[3,2-c]pyridin-4-one trifluoroacetate | 6.47 | |
| 766 | 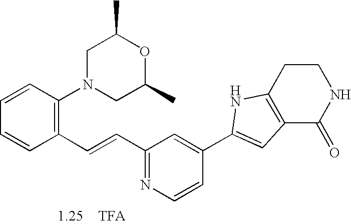 | 2-[2-((E)-2-{2-[(2R,6S)-2,6-dimethylmorpholin-4- yl]phenyl}vinyl)pyridin-4-yl]-1,5,6,7-tetrahydro-4H-pyrrolo[3,2- c]pyridin-4-one trifluoroacetate | 7.54 | |
| 767 | 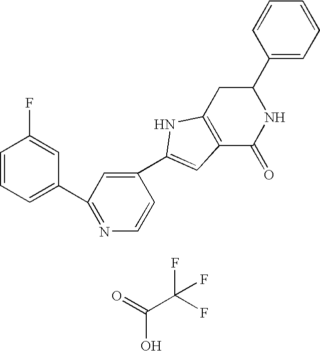 | 2-[2-(3-fluorophenyl)pyridin-4-yl]-6-phenyl-1,5,6,7-tetrahydro-4H- pyrrolo[3,2-c]pyridin-4-one trifluoroacetate | 7.75 | |
| 768 | 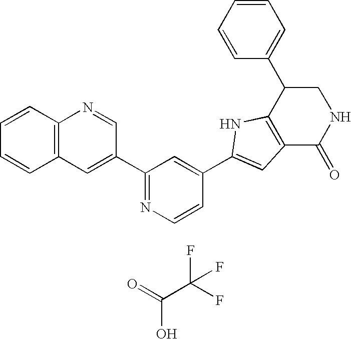 | 7-phenyl-2-(2-quinolin-3-ylpyridin-4-yl)-1,5,6,7-tetrahydro-4H- pyrrolo[3,2-c]pyridin-4-one trifluoroacetate | 7.98 | |
| 769 | 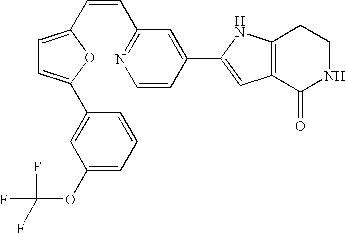 | 2-[2-((Z)-2-{5-(3-(trifluoromethoxy)phenyl]-2-furyl}vinyl)pyridin-4- yl]-1,5,6,7-tetrahydro-4H-pyrrolo[3,2-c]pyridin-4-one | 8.18 | |
| 770 | 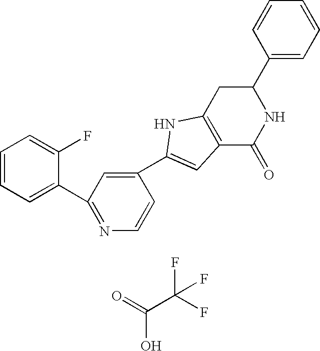 | 2-[2-(2-fluorophenyl)pyridin-4-yl]-6-phenyl-1,5,6,7-tetrahydro-4H- pyrrolo[3,2-c]pyridin-4-one trifluoroacetate | 8.32 | |
| 771 | 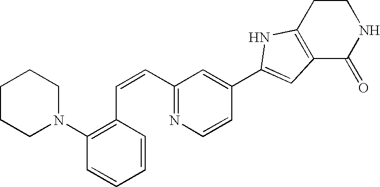 | 2-{2-[(Z)-2-(2-piperidin-1-ylphenyl)vinyl]pyridin-4-yl}-1,5,6,7- tetrahydro-4H-pyrrolo[3,2-c]pyridin-4-one | 8.36 | |
| 772 | 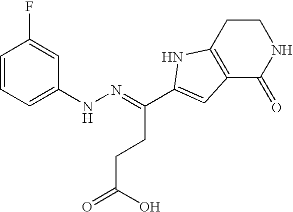 | (4E)-4-[(3-fluorophenyl)hydrazono]-4-(4-oxo-4,5,6,7-tetrahydro 1H-pyrrolo[3,2-c]pyridin-2-yl)butanoic acid | 8.44 | |
| 773 | 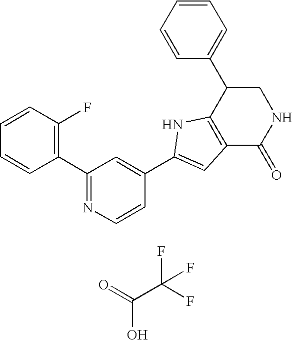 | 2-[2-(2-fluorophenyl)pyridin-4-yl]-7-phenyl-1,5,6,7-tetrahydro-4H- pyrrolo[3,2-c]pyridin-4-one trifluoroacetate | 8.62 | |
| 774 | 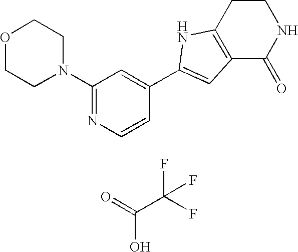 | 2-(2-morpholin-4-ylpyridin-4-yl)-1,5,6,7-tetrahydro-4H- pyrrolo[3,2-c]pyridin-4-one trifluoroacetate | 8.8 | |
| 775 | 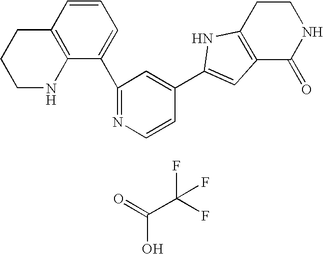 | 2-[2-(1,2,3,4-tetrahydroquinolin-8-yl)pyridin-4-yl]-1,5,6,7- tetrahydro-4H-pyrrolo[3,2-c]pyridin-4-one trifluoroacetate | 9.04 | |
| 776 | 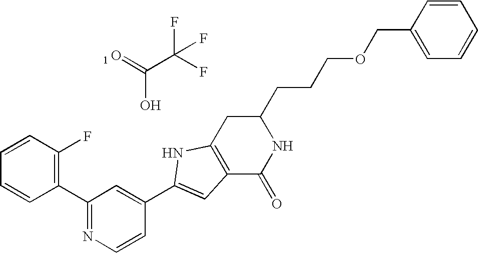 | 6-[3-(benzyloxy)propyl]-2-[2-(2-fluorophenyl)pyridin-4-yl]-1,5,6,7- tetrahydro-4H-pyrrolo[3,2-c]pyridin-4-one trifluoroacetate | 9.32 | |
| 777 | 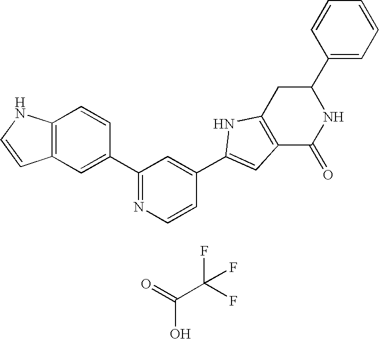 | 2-[2-(1H-indol-5-yl)pyridin-4-yl]-6-phenyl-1,5,6,7-tetrahydro-4H- pyrrolo[3,2-c]pyridin-4-one trifluoroacetate | 9.6 | |
| 778 | 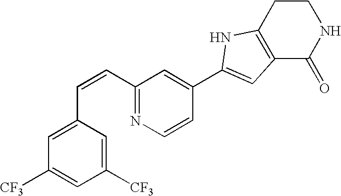 | 2-(2-{(Z)-2-[3,5-bis(trifluoromethyl)phenyl]vinyl}pyridin-4-yl)- 1,5,6,7-tetrahydro-4H-pyrrolo[3,2-c]pyridin-4-one | 9.91 | |
| 779 | 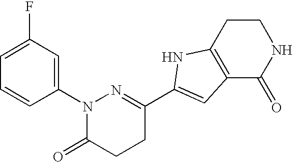 | 2-[1-(3-fluorophenyl)-6-oxo-1,4,5,6-tetrahydropyridazin-3-yl]- 1,5,6,7-tetrahydro-4H-pyrrolo[3,2-c]pyridin-4-one | 10.4 | |
| 780 | 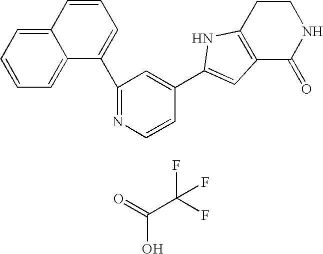 | 2-(2-quinolin-8-ylpyridin-4-yl)-1,5,6,7-tetrahydro-4H-pyrrolo[3,2- c]pyridin-4-one trifluoroacetate | 10.8 | |
| 781 | 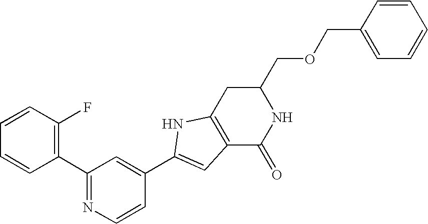 | 6-[(benzyloxy)methyl]-2-[2-(2-fluorophenyl)pyridin-4-yl]-1,5,6,7- tetrahydro-4H-pyrrolo[3,2-c]pyridin-4-one | 11 | |
| 782 | 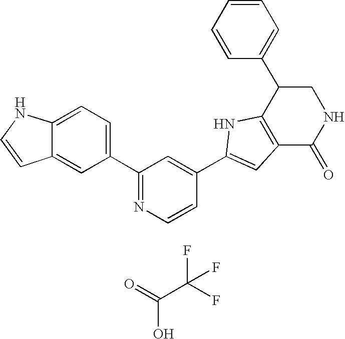 | 2-[2-(1H-indol-5-yl)pyridin-4-yl]-7-phenyl-1,5,6,7-tetrahydro-4H- pyrrolo[3,2-c]pyridin-4-one trifluoroacetate | 11.5 | |
| 783 | 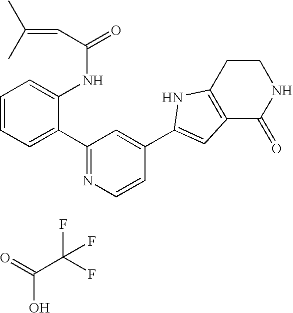 | 3-methyl-N-{2-[4-(4-oxo-4,5,6,7-tetrahydro-1H-pyrrolo[3,2- c]pyridin-2-yl)pyridin-2-yl]phenyl}but-2-enamide trifluoroacetate | 12 | |
| 784 | 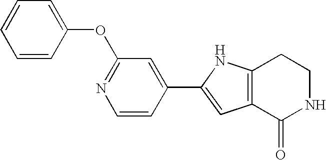 | 2-(2-phenoxypyridin-4-yl)-1,5,6,7-tetrahydro-4H-pyrrolo[3,2- c]pyridin-4-one | 12.1 | |
| 785 | 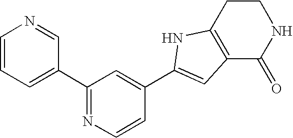 | 2-(2,3′-bipyridin-4-yl)-1,5,6,7-tetrahydro-4H-imidazo[4,5- c]pyridin-4-one | 12.5 | |
| 786 | 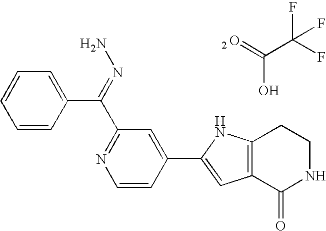 | 2-{2-[(E)-hydrazono(phenyl)methyl]pyridin-4-yl}-1,5,6,7- tetrahydro-4H-pyrrolo[3,2-c]pyridin-4-one trifluoroacetate | 14.3 | |
| 787 | 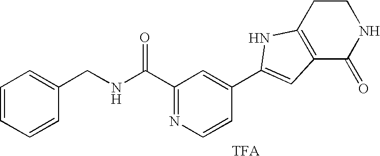 | N-benzyl-4-(4-oxo-4,5,6,7-tetrahydro-1H-pyrrolo[3,2-c]pyridin-2- yl)pyridine-2-carboxamide trifluoroacetate | 14.4 | |
| 788 | 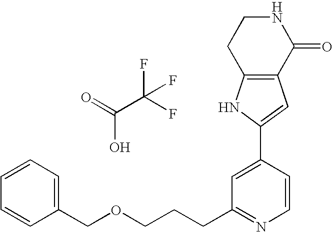 | 2-{2-(3-(benzyloxy)propyl]pyridin-4-yl}-1,5,6,7-tetrahydro-4H- pyrrolo[3,2-c]pyridin-4-one trifluoroacetate or 2-{2-[3- (benzyloxy)propyl]pyridin-4-yl}-1,5,6,7-tetrahydro-4H-pyrrolo[3,2- c]pyridin-4-one trifluoroacetate | 16.2 | |
| 789 | 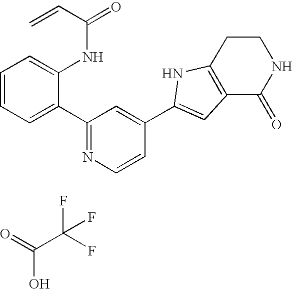 | N-{2-[4-(4-oxo-4,5,6,7-tetrahydro-1H-pyrrolo[3,2-c]pyridin-2- yl)pyridin-2-yl]phenyl}acrylamide trifluoroacetate | 16.5 | |
| 790 | 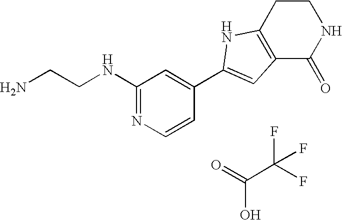 | 2-{2-((2-aminoethyl)amino]pyridin-4-yl}-1,5,6,7-tetrahydro-4H- pyrrolo[3,2-c]pyridin-4-one trifluoroacetate | 17 | |
| 791 | 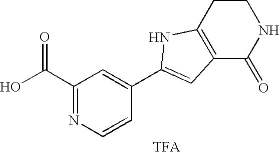 | 4-(4-oxo-4,5,6,7-tetrahydro-1H-pyrrolo[3,2-c]pyridin-2- yl)pyridine-2-carboxylic acid trifluoroacetate | 19.1 | |
| 792 | 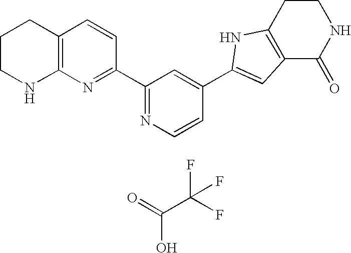 | 2-[3-(5,6,7,8-tetrahydro-1,8-naphthyridin-2-yl)phenyl]-1,5,6,7- tetrahydro-4H-pyrrolo[3,2-c]pyridin-4-one trifluoroacetate | 20 | |
| 793 | 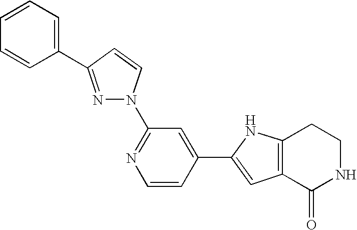 | 2-[2-(3-phenyl-1H-pyrazol-1-yl)pyridin-4-yl]-1,5,6,7-tetrahydro- 4H-pyrrolo[3,2-c]pyridin-4-one | 20 | |
| 794 | 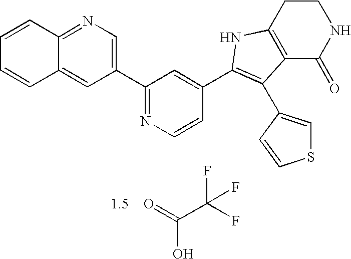 | 2-(2-quinolin-3-ylpyridin-4-yl)-3-thien-3-yl-1,5,6,7-tetrahydro-4H- pyrrolo[3,2-c]pyridin-4-one trifluoroacetate | 20 | |
| 795 | 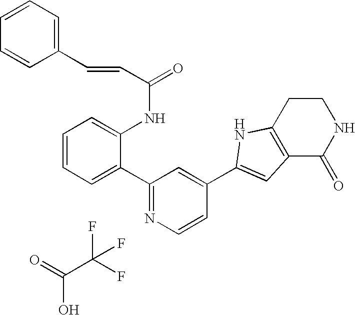 | (2E)-N-{2-[4-(4-oxo-4,5,6,7-tetrahydro-1H-pyrrolo[3,2-c]pyridin- 2-yl)pyridin-2-yl]phenyl}-3-phenylacrylamide trifluoroacetate | 20 | |
| 796 | 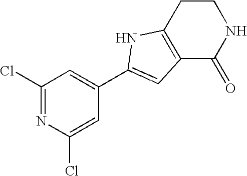 | 2-(2,6-dichloropyridin-4-yl)-1,5,6,7-tetrahydro-4H-pyrrolo[3,2- c]pyridin-4-one | 20 | |
| 797 | 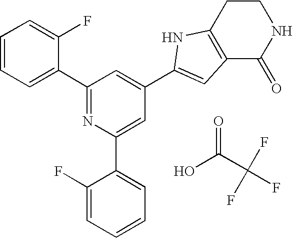 | 2-[2,6-bis(2-fluorophenyl)pyridin-4-yl]-1,5,6,7-tetrahydro-4H- pyrrolo[3,2-c]pyridin-4-one trifluoroacetate | 20 | |
| 798 | 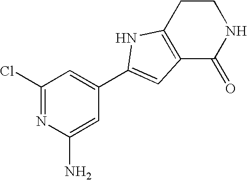 | 2-(2-amino-6-chloropyridin-4-yl)-1,5,6,7-tetrahydro-4H- pyrrolo[3,2-c]pyridin-4-one | 20 | |
| 799 | 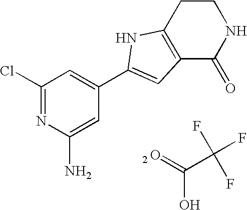 | 2-(2-amino-6-chloropyridin-4-yl)-1,5,6,7-tetrahydro-4H- pyrrolo[3,2-c]pyridin-4-one trifluoroacetate | 20 | |
| 800 | 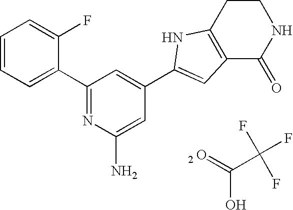 | 2-[2-amino-6-(2-fluorophenyl)pyridin-4-yl]-1,5,6,7-tetrahydro-4H- pyrrolo[3,2-c]pyridin-4-one trifluoroacetate | 20 | |
| 801 | 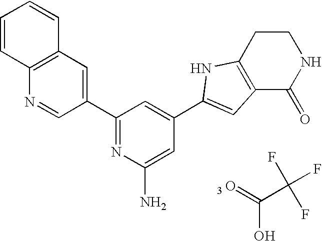 | 2-(2-amino-6-quinoIin-3-ylpyridin-4-yl)-1,5,6,7-tetrahydro-4H- pyrrolo[3,2-c]pyridin-4-one trifluoroacetate | 20 | |
| 802 | 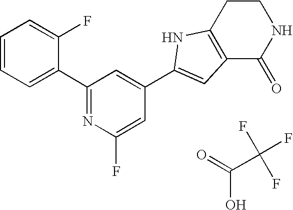 | 2-[2-fluoro-6-(2-fluorophenyl)pyridin-4-yl]-1,5,6,7-tetrahydro-4H- pyrrolo[3,2-c]pyridin-4-one trifluoroacetate | 20 | |
| 803 | 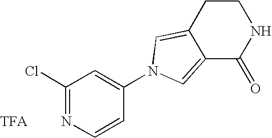 | 2-(2-chloropyridin-4-yl)-2,5,6,7-tetrahydro-4H-pyrrolo[3,4- c]pyridin-4-one trifluoroacetate | 20 | |
| 804 | 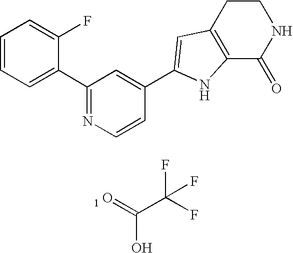 | 2-[2-(2-fluorophenyl)pyridin-4-yl]-1,4,5,6-tetrahydro-7H- pyrrolo[2,3-c]pyridin-7-one trifluoroacetate | 20 | |
| 805 | 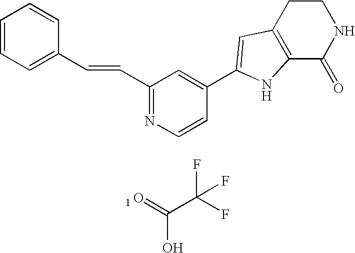 | 2-{2-[(E)-2-phenylethenyl]pyridin-4-yl}-1,4,5,6-tetrahydro-7H- pyrrolo[2,3-c]pyrid,n-7-one trifluoroacetate | 20 | |
| 806 | 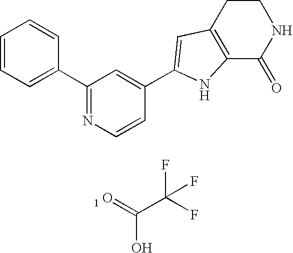 | 2-(2-phenylpyridin-4-yl)-1,4,5,6-tetrahydro-7H-pyrrolo[2,3- c]pyridin-7-one trifluoroacetate | 20 | |
| 807 | 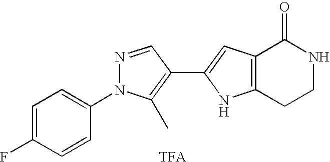 | 2-[1-(4-fluorophenyl)-5-methyl-1H-pyrazol-4-yl]-1,5,6,7- tetrahydro-4H-pyrrolo[3,2-c]pyridin-4-one trifluoroacetate | 20 | |
| 808 | 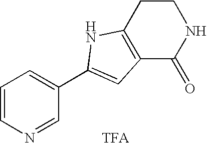 | 2-pyridin-3-yl-1,5,6,7-tetrahydro-4H-pyrrolo[3,2-c]pyridin-4-one trifluoroacetate | 20 | |
| 809 | 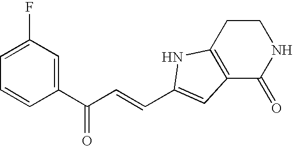 | 2-[(1E)-3-(3-fluorophenyl)-3-oxoprop-1-enyl]-1,5,6,7-tetrahydro- 4H-pyrrolo[3,2-c]pyridin-4-one | 20 | |
| 810 | 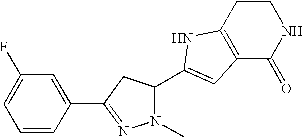 | 2-[3-(3-fluorophenyl)-1-methyl-4,5-dihydro-1H-pyrazol-5-yl]- 1,5,6,7-tetrahydro-4H-pyrrolo[3,2-c]pyridin-4-one | 20 | |
| 811 | 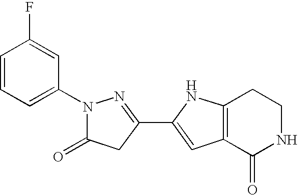 | 2-[1-(3-fluorophenyl)-5-oxo-4,5-dihydro-1H-pyrazol-3-yl]-1,5,6,7- tetrahydro-4H-pyrrolo[3,2-c]pyridin-4-one | 20 | |
| 812 | 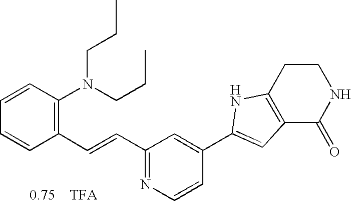 | 2-(2-{(E)-2-[2-(dipropylamino)phenyl]vinyl}pyridin-4-yl)-1,5,6,7- tetrahydro-4H-pyrroio[3,2-c]pyridin-4-one trifluoroacetate | 20 | |
| 813 | 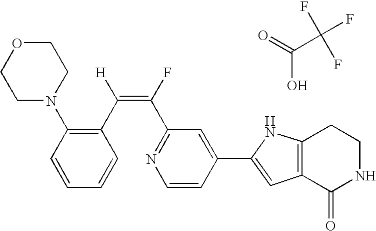 | 2-{2-[(E)-1-fluoro-2-(2-morpholin-4-ylphenyl)vinyl]pyridin-4-yl}- 1,5,6,7-tetrahydro-4H-pyrrolo[3,2-c]pyridin-4-one trifluoroacetate | 20 | |
| 814 | 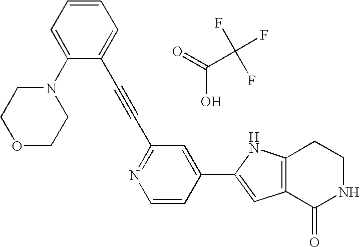 | 2-{2-[(2-morpholin-4-ylphenyl)ethynyl]pyridin-4-yl}-1,5,6,7- tetrahydro-4H-pyrrolo[3,2-c]pyridin-4-one trifluoroacetate | 20 | |
| 815 | 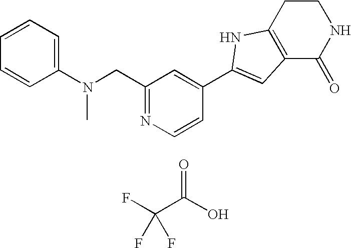 | N-methyl-4-(4-oxo-4,5,6,7-tetrahydro-1H-pyrrolo[3,2-c]pyridin-2- yl)-N-phenylpyridine-2-carboxamide trifluoroacetate | 20 | |
| 816 | 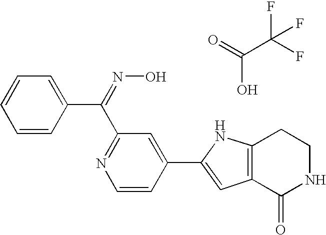 | 2-{2-[(Z)-(hydroxyimino)(phenyl)methyl]pyridin-4-yl}-1,5,6,7- tetrahydro-4H-pyrrolo[3,2-c]pyridin-4-one trifluoroacetate | 20 | |
| 817 | 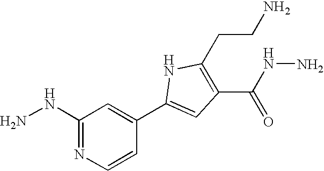 | 2-(2-aminoethyl)-5-(2-hydrazinopyridin-4-yl)-1H-pyrrole-3- carbohydrazide | 20 | |
| 818 | 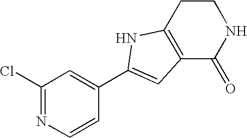 | 2-(2-chloropyridin-4-yl)-1,5,6,7-tetrahydro-4H-pyrrolo[3,2- c]pyridin-4-one | | |
| 819 | 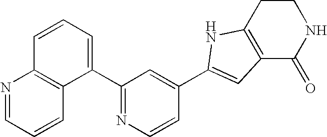 | 2-(2-quinolin-5-ylpyridin-4-yl)-1,5,6,7-tetrahydro-4H-pyrrolo[3,2- c]pyridin-4-one | | |
| 820 | 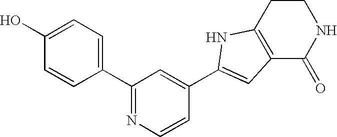 | 2-[2-(4-hydroxyphenyl)pyridin-4-yl]-1,5,6,7-tetrahydro-4H- pyrrolo[3,2-c]pyridin-4-one | | |
| 821 | 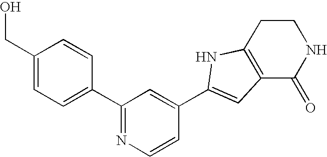 | 2-{2-[4-(hydroxymethyl)phenyl]pyridin-4-yl}-1,5,6,7-tetrahydro- 4H-pyrrolo[3,2-c]pyridin-4-one | | |
| 822 | 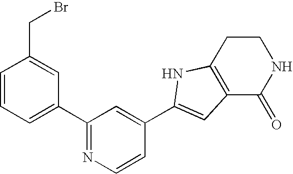 | 2-{2-[3-(bromomethyl)phenyl]pyridin-4-yl}-1,5,6,7-tetrahydro-4H- pyrrolo[3,2-c]pyridin-4-one | | |
| 823 | 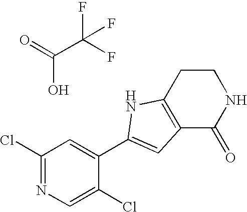 | 2-(2,5-dichloropyridin-4-yl)-1,5,6,7-tetrahydro-4H-pyrrolo[3,2- c]pyridin-4-one trifluoroacetate | | |
| 824 | 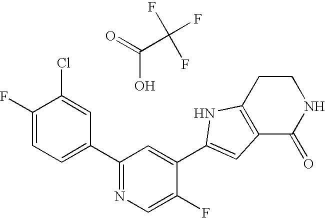 | 2-[2-(3-chloro-4-fluorophenyl)-5-fluoropyridin-4-yl]-1,5,6,7- tetrahydro-4H-pyrrolo[3,2-c]pyridin-4-one trifluoroacetate | | |
| 825 | 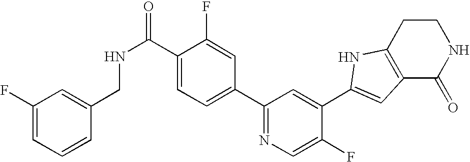 | 2-fluoro-N-(3-fluorobenzyl)-4-[5-fluoro-4-(4-oxo-4,5,6,7- tetrahydro-1H-pyrrolo[3,2-c]pyridin-2-yl)pyridin-2-yl]benzamide | | |
| 826 | 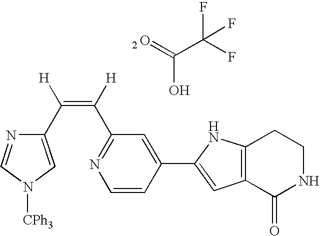 | 2-{2-[(Z)-2-(1-trityl-1H-imidazoi-4-yl)ethenyl]pyridin-4-yl}-1,5,6,7- tetrahydro-4H-pyrrolo[3,2-c]pyridin-4-one trifluoroacetate | | |
| 827 | 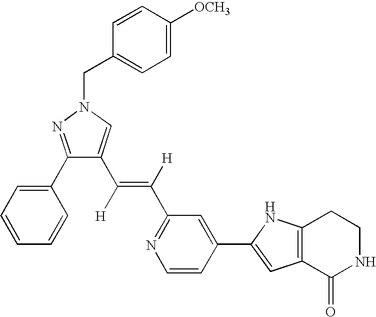 | 2-(2-{(E)-2-[1-(4-methoxybenzyl)-3-phenyl-1H-pyrazol-4- yl]vinyl}pyridin-4-yl)-1,5,6,7-tetrahydro-4H-pyrralo[3,2-c]pyridin- 4-one | | |
| 828 | 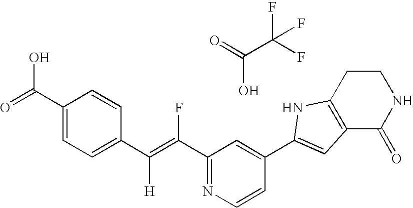 | 4-{(Z)-2-fluoro-2-[4-(4-oxo-4,5,6,7-tetrahydro-1H-pyrrolo(3,2- c]pyridin-2-yl)pyridin-2-yl]vinyl}benzoic acid trifluoroacetate | | |
| 829 | 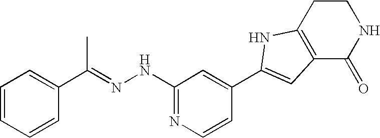 | 2-{2-[(2E)-2-(1-phenylethylidene)hydrazino]pyridin-4-yl}-1,5,6,7- tetrahydro-4H-pyrrolo[3,2-c]pyridin-4-one | | |
| 830 | 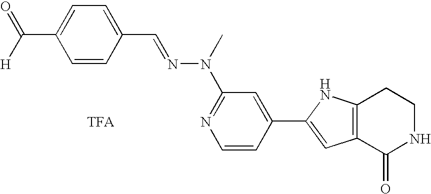 | terephthalaldehyde methyl[4-(4-oxo-4,5,6,7-tetrahydro-1H- pyrrolo[3,2-c]pyridin-2-yl)pyridin-2-yl]hydrazone trifluoroacetate | | |
| 831 | 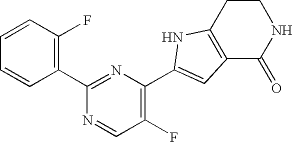 | 2-[5-fluoro-2-(2-fluorophenyl)pyrimidin-4-yl]-1,5,6,7-tetrahydro- 4H-pyrrolo[3,2-c]pyridin-4-one | 0.0635 | |

Notes:
^{a}Chemical names were generated by ACD/Name software.
^{b}The MK-2 inhibiting compound may be shown with a solvent, such as, for example, trifluoroacetate, with which it can form a salt. Both the salt and base forms of the pyrrole compound are included in the present invention.

TABLE II
Examples of MK-2 inhibiting compounds; Structure and Name.

| Number | Structure^{a} | Compound Name(s)^{b} |
|---|---|---|
| 832 | 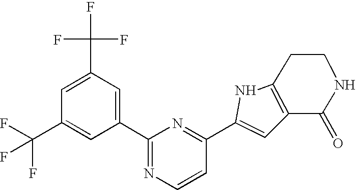 | 2-{2-[3,5-bis(trifluoromethyl)phenyl]pyrimidin-4-yl]-1,5,6,7- tetrahydro-4H-pyrroio[3,2-c]pyridin-4-one |
| 833 | 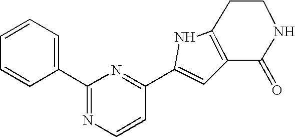 | 2-(2-phenylpyrimidin-4-yl)-1,5,6,7-tetrahydro-4H- pyrrolo[3,2-c]pyridin-4-one |
| 834 | 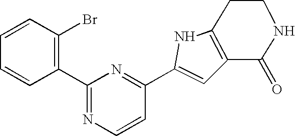 | 2-[2-(2-bromophenyl)pyrimidin-4-yl]-1,5,6,7-tetrahydro-4H- pyrrolo[3,2-c]pyridin-4-one |
| 835 | 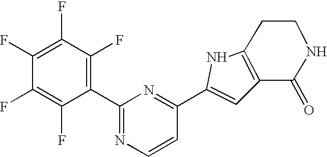 | 2-[2-(pentafluorophenyl)pyrimidin-4-yl]-1,5,6,7-tetrahydro- 4H-pyrrolo[3,2-c]pyridin-4-one |
| 836 | 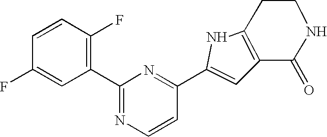 | 2-[2-(2,5-difluorophenyl)pyrimidin-4-yl]-1,5,6,7-tetrahydro- 4H-pyrrolo[3,2-c]pyridin-4-one |
| 837 | 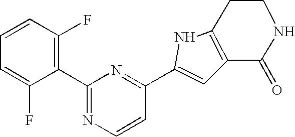 | 2-[2-(2,6-difluorophenyl)pyrimidin-4-yl]-1,5,6,7-tetrahydro- 4H-pyrrolo[3,2-c]pyridin-4-one |
| 838 | 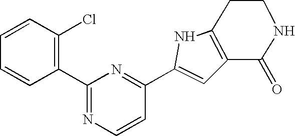 | 2-[2-(2-chlorophenyl)pyrimidin-4-yl]-1,5,6,7-tetrahydro-4H- pyrrolo[3,2-c]pyridin-4-one |
| 839 | 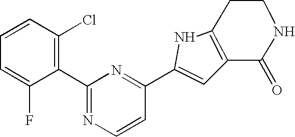 | 2-[2-(2.chloro-6-fluorophenyl)pyrimidin-4-yl]-1,5,6,7- tetrahydro-4H-pyrrolo[3,2-c]pyridin-4-one |
| 840 | 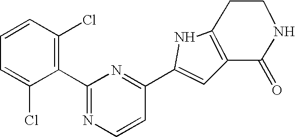 | 2-[2-(2,6-dichlorophenyl)pyrimidin-4-yl]-1,5,6,7-tetrahydro- 4H-pyrrolo[3,2-c]pyridin-4-one |
| 841 | 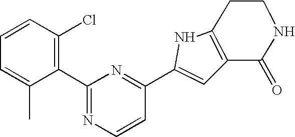 | 2-[2-(2-chloro-6-methylphenyl)pyrimidin-4-yl]-1,5,6,7- tetrahydro-4H-pyrrolo[3,2-c]pyridin-4-one |
| 842 | 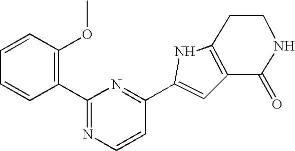 | 2-[2-(2-methoxyphenyl)pyrimidin-4-yl]-1,5,6,7-tetrahydro- 4H-pyrrolo[3,2-c]pyridin-4-one |
| 843 | 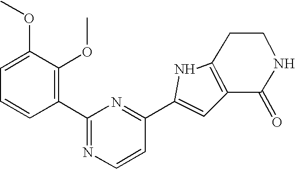 | 2-[2-(2,3-dimethoxyphenyl)pyrimidin-4-yl]-1,5,6,7- tetrahydro-4H-pyrrolo[3,2-c]pyridin-4-one |
| 844 | 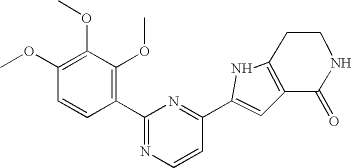 | 2-[2-(2,3,4-trimethoxyphenyl)pyrimidin-4-yl]-1,5,6,7- tetrahydro-4H-pyrroio[3,2-c]pyridin-4-one |
| 845 | 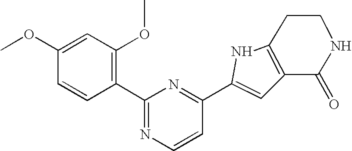 | 2-[2-(2,4-dimethoxyphenyl)pyrimidin-4-yl]-1,5,6,7- tetrahydro-4H-pyrrolo[3,2-c]pyridin-4-one |
| 846 | 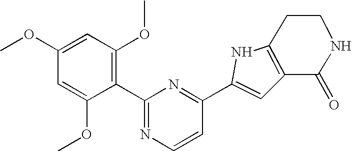 | 2-[2-(2,4,6-trimethoxyphenyl)pyrimidin-4-yl]-1,5,6,7- tetrahydro-4H-pyrrolo[3,2-c]pyridin-4-one |
| 847 | 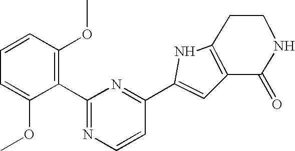 | 2-[2-(2,6-dimethoxyphenyl)pyrimidin-4-yl]-1,5,6,7- tetrahydro-4H-pyrrolo[3,2-c]pyridin-4-one |
| 848 | 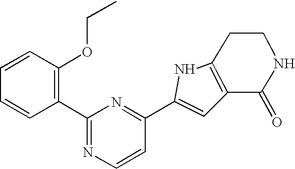 | 2-[2-(2-ethoxyphenyl)pyrimidin-4-yl]-1,5,6,7-tetrahydro-4H- pyrrolo[3,2-c]pyridin-4-one |
| 849 | 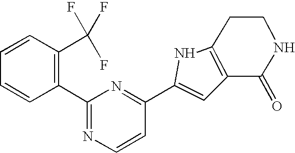 | 2-{2-[2-(trifiuoromethyl)phenyl]pyrimidin-4-yl]56 -1,5,6,7- tetrahydro-4H-pyrrolo[3,2-c]pyridin-4-one |
| 850 | 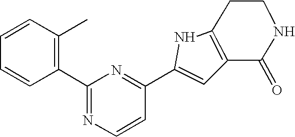 | 2-[2-(2-methylphenyl)pyrimidin-4-yl]-1,5,6,7-tetrahydro-4H- pyrrolo[3,2-c]pyridin-4-one |
| 851 | 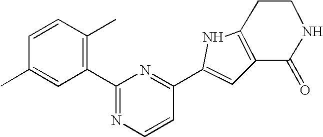 | 2-[2-(2,5-dimethylphenyl)pyrimidin-4-yl]-1,5,6,7-tetrahydro- 4H-pyrrolo[3,2-c]pyridin-4-one |
| 852 | 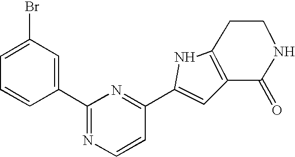 | 2-[2-(3-bromophenyl)pyrimidin-4-yl]-1,5,6,7-tetrahydro-4H- pyrrolo[3,2-c]pyridin-4-one |
| 853 | 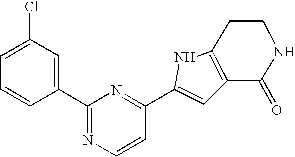 | 2-[2-(3-chlorophenyl)pyrimidin-4-yl]-1,5,6,7-tetrahydro-4H- pyrrolo[3,2-c]pyridin-4-one |
| 854 | 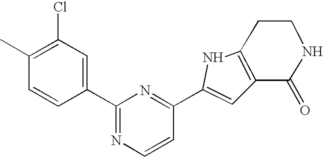 | 2-[2-(3-chloro-4-methylphenyl)pyrimidin-4-yl]-1,5,6,7- tetrahydro-4H-pyrroio[3,2-c]pyridin-4-one |
| 855 | 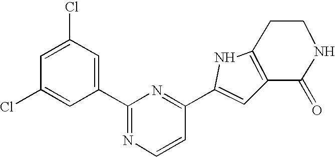 | 2-[2-(3,5-dichlorophenyl)pyrimidin-4-yl]-1,5,6,7-tetrahydro- 4H-pyrrolo[3,2-c]pyridin-4-one |
| 856 | 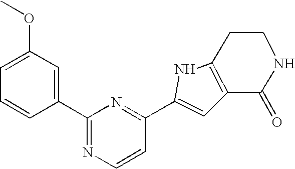 | 2-[2-(3-methoxyphenyl)pyrimidin-4-yl]-1,5,6,7-tetrahydro- 4H-pyrrolo[3,2-c]pyridin-4-one |
| 857 | 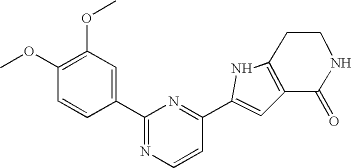 | 2-[2-(3,4-dimethoxyphenyl)pyrimidin-4-yl]-1,5,6,7- tetrahydro-4H-pyrrolo[3,2-c]pyridin-4-one |
| 858 | 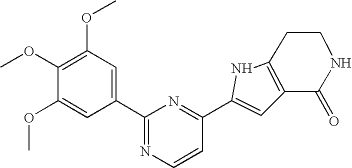 | 2-[2-(3,45-trimethoxyphenyl)pyrimidin-4-yl]-1,5,6,7- tetrahydro-4H-pyrrolo[3,2-c]pyridin-4-one |
| 859 | 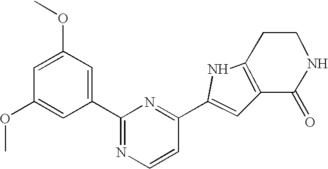 | 2-[2-(35-dimethoxyphenyl)pyrimidin-4-yl]-1,5,6,7- tetrahydro-4H-pyrrolo[3,2-c]pyridin-4-one |
| 860 | 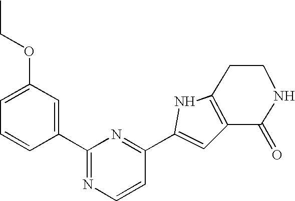 | 2-[2-(3-ethoxyphenyl)pyrimidin-4-yl]-1,5,6,7-tetrahydro-4H- pyrrolo[3,2-c]pyridin-4-one |
| 861 | 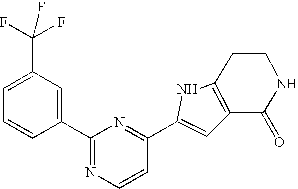 | 2-{2-[3-(trifluoromethyl)phenyl]pyrimidin-4-yl]-1,5,6,7- tetrahydro-4H-pyrrolo[3,2-c]pyridin-4-one |
| 862 | 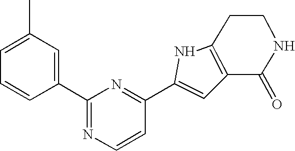 | 2-[2-(3-methylphenyl)pyrimidin-4-yl]-1,5,6,7-tetrahydro-4H- pyrrolo-[3,2-c]pyridin-4-one |
| 863 | 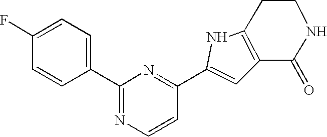 | 2-[2-(4-fluorophenyl)pyrimidin-4-yl]-1,5,6,7-tetrahydro-4H- pyrrolo[3,2-c]pyridin-4-one |
| 864 | 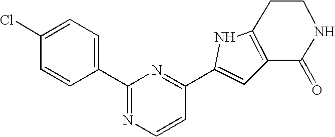 | 2-[2-(4-chlorophenyl)pyrimidin-4-yl]-1,5,6,7-tetrahydro-4H- pyrrolo[3,2-c]pyridin-4-one |
| 865 | 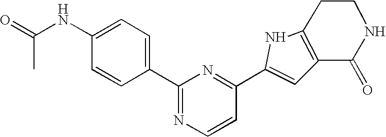 | N-{4-[4-(4-oxo-4,5,6,7-tetrahydro-1H-pyrrolo[3,2-c]pyridin- 2-yl)pyrimidin-2-yl]phenyl]56 acetamide |
| 866 | 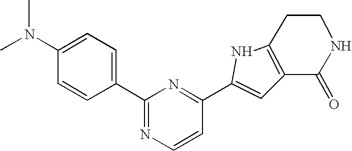 | 2-{2-[4-(dimethylamino)phenyl]pyrimidin-4-yl]-1,5,6,7- tetrahydro-4H-pyrrolo[3,2-c]pyridin-4-one |
| 867 | 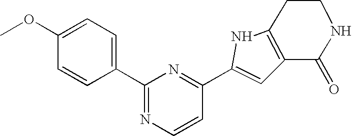 | 2-[2-(4-methoxyphenyl)pyrimidin-4-yl]-1,5,6,7-tetrahydro- 4H-pyrrolo[3,2-c]pyridin-4-one |
| 868 | 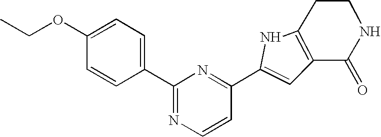 | 2-[2-(4-ethoxyphenyl)pyrimidin-4-yl]-1,5,6,7-tetrahydro-4H- pyrrolo[3,2-c]pyridin-4-one |
| 869 | 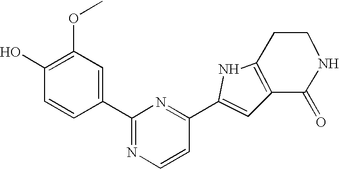 | 2-[2-(4-hydroxy-3-methoxyphenyl)pyrimidin-4-yl]-1,5,6,7- tetrahydro-4H-pyrrolo[3,2-c]pyridin-4-one |
| 870 | 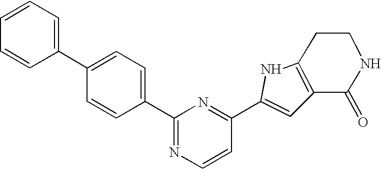 | 2-[2-(1,1′-biphenyl-4-yl)pyrimidin-4-yl]-1,5,6,7-tetrahydro- 4H-pyrrolo[3,2-c]pyridin-4-one |
| 871 | 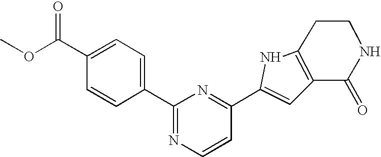 | methyl 4-[4-(4-oxo-4,5,6,7-tetrahydro-1H-pyrrolo[3,2- c]pyridin-2-yl)pyrimidin-2-yl]benzoate |
| 872 | 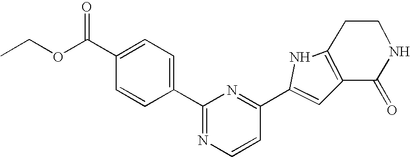 | ethyl 4-[4-(4-oxo-4,5,6,7-tetrahydro-1H-pyrrolo[3,2- c]pyridin-2-yl)pyrimidin-2-yl]benzoate |
| 873 | 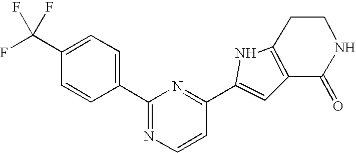 | 2-{2-{4-(trifluoromethyl)phenyl]pyrimidin-4-yl]56 -1,5,6,7- tetrahydro-4H-pyrrolo[3,2-c]pyridin-4-one |
| 874 | 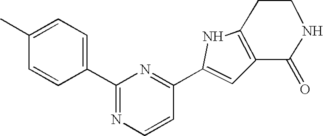 | 2-[2-(4-methylphenyl)pyrimidin-4-yl]-1,5,6,7-tetrahydro-4H- pyrrolo[3,2-c]pyridin-4-one |
| 875 | 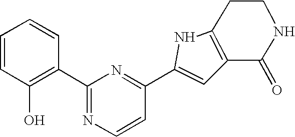 | 2-[2-(2-hydroxyphenyl)pyrimidin-4-yl]-1,5,6,7-tetrahydro- 4H-pyrrolo[3,2-c]pyridin-4-one |
| 876 | 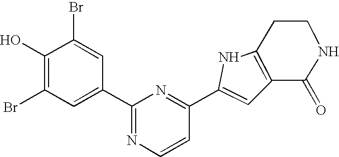 | 2-[2-(3,5-dibromo-4-hydroxyphenyl)pyrimidin-4-yl]-1,5,6,7- tetrahydro-4H-pyrrolo[3,2-c]pyridin-4-one |
| 877 | 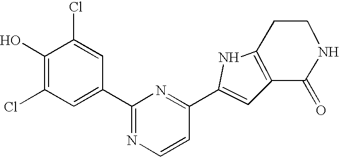 | 2-[2-(3,5-dichloro-4-hydroxyphenyl)pyrimidin-4-yl]-1,5,6,7- tetrahydro-4H-pyrrolo[3,2-c]pyridin-4-one |
| 878 | 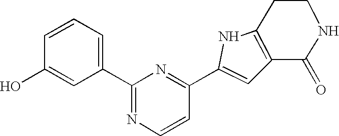 | 2-[2-(3-hydroxyphenyl)pyrimidin-4-yl]-1,5,6,7-tetrahydro- 4H-pyrrolo[3,2-c]pyridin-4-one |
| 879 | 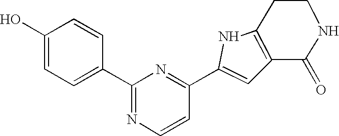 | 2-[2-(4-hydroxyphenyl)pyrimidin-4-yl]-1,5,6,7-tetrahydro- 4H-pyrrolo[3,2-c]pyridin-4-one |
| 880 | 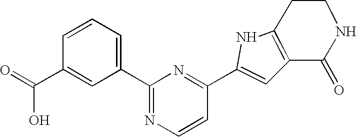 | 3-[4-(4-oxo-4,5,6,7-tetrahydro-1H-pyrrolo[3,2-c]pyridin-2- yl)pyrimidin-2-yl]benzoic acid |
| 881 | 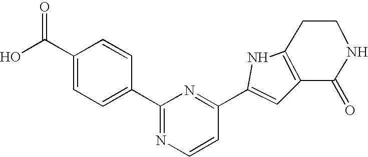 | 4-[4-(4-oxo-4,5,6,7-tetrahydro-1H-pyrrolo[3,2-c]pyridin-2- yl)pyrimidin-2-yl]benzoic acid |
| 882 | 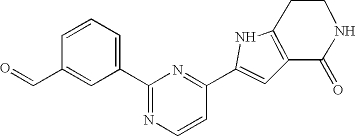 | 3-[4-(4-oxo-4,5,6,7-tetrahydro-1H-pyrrolo[3,2-c]pyridin-2- yl)pyrimidin-2-yl]benzaldehyde |
| 883 | 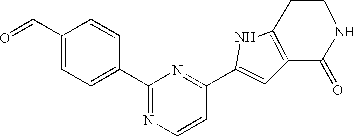 | 4-[4-(4-oxo-4,5,6,7-tetrahydro-1H-pyrrolo[3,2-c]pyridin-2- yl)pyrimidin-2-yl]benzaldehyde |
| 884 | 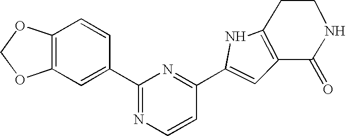 | 2-[2-(1,3-benzodioxol-5-yl)pyrimidin-4-yl]-1,5,6,7- tetrahydro-4H-pyrrolo[3,2-c]pyridin-4-one |
| 885 | 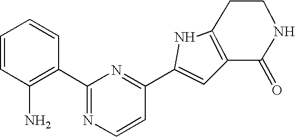 | 2-[2-(2-aminophenyl)pyrimidin-4-yl]-1,5,6,7-tetrahydro-4H- pyrrolo[3,2-c]pyridin-4-one |
| 886 | 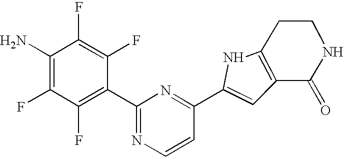 | 2-[2-(4-amino-2,3,5,6-tetrafluorophenyl)pyrimidin-4-yl]- 1,5,6,7-tetrahydro-4H-pyrrolo[3,2-c]pyridin-4-one |
| 887 | 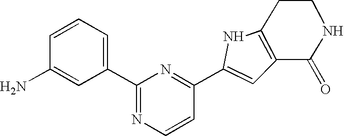 | 2-[2-(3-aminophenyl)pyrimidin-4-yl]-1,5,6,7-tetrahydro-4H- pyrrolo[3,2-c]pyridin-4-one |
| 888 | 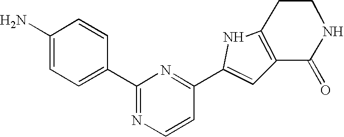 | 2-[2-(4-aminophenyl)pyrimidin-4-yl]-1,5,6,7-tetrahydro-4H- pyrrolo[3,2-c]pyridin-4-one |
| 889 | 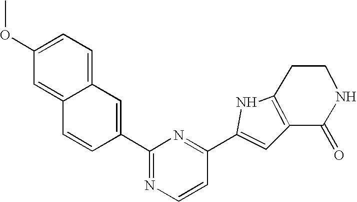 | 2-[2-(6-methoxy-2-naphthyl)pyrimidin-4-yl]-1,5,6,7- tetrahydro-4H-pyrrolo[3,2-c]pyridin-4-one |
| 890 | 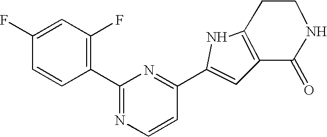 | 2-[2-(2,3-difluorophenyl)pyrimidin-4-yl]-1,5,6,7-tetrahydro- 4H-pyrrolo[3,2-c]pyridin-4-one |
| 891 | 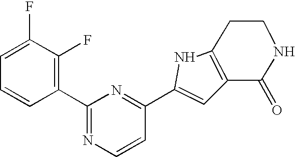 | 2-[2-(3,5-difluorophenyl)pyrimidin-4-yl]-1,5,6,7-tetrahydro- 4H-pyrrolo[3,2-c]pyridin-4-one |
| 892 | 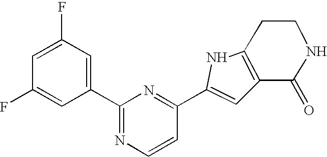 | 2-[2-(3,5-difluorophenyl)pyrimidin-4-yl]-1,5,6,7-tetrahydro- 4H-pyrrolo[3,2-c]pyridin-4-one |
| 893 | 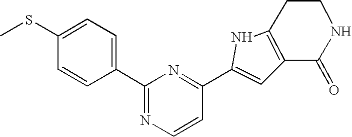 | 2-{2-[4-(methylthio)phenyl]pyrimidin-4-yl]-1,5,6,7- tetrahydro-4H-pyrrolo[3,2-c]pyridin-4-one |
| 894 | 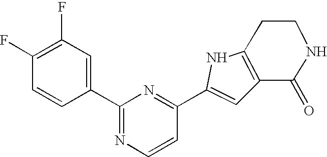 | 2-[2-(3,4-difluorophenyl)pyrimidin-4-yl]-1,5,6,7-tetrahydro- 4H-pyrrolo[3,2-c]pyridin-4-one |
| 895 | 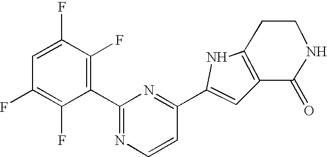 | 2-[2-(2,3,5,6-tetrafluorophenyl)pyrimidin-4-yl]-1,5,6,7- tetrahydro-4H-pyrrolo[3,2-c]pyridin-4-one |
| 896 | 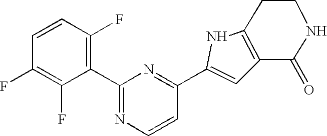 | 2-[2-(2,3,6-trifluorophenyl)pyrimidin-4-yl]-1,5,6,7- tetrahydro-4H-pyrrolo[3,2-c]pyridin-4-one |
| 897 | 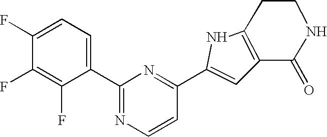 | 2-[2-(2,3,4-trifluorophenyl)pyrimidin-4-yl]-1,5,6,7- tetrahydro-4H-pyrrolo[3,2-c]pyridin-4-one |
| 898 | 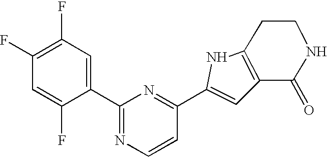 | 2-[2-(2,4,5-trifluorophenyl)pyrimidin-4-yl]-1,5,6,7- tetrahydro-4H-pyrrolo[3,2-c]pyridin-4-one |
| 899 | 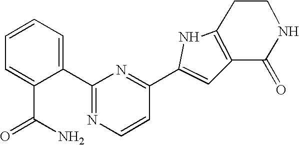 | 2-[4-(4-oxo-4,5,6,7-tetrahydro-1H-pyrrolo[3,2-c]pyridin-2- yl)pyrimidin-2-yl]benzamide |
| 900 | 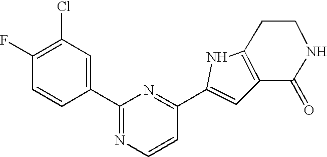 | 2-[2-(3-chloro-4-fluorophenyl)pyrimidin-4-yl]-1,5,6,7- tetrahydro-4H-pyrrolo[3,2-c]pyridin-4-one |
| 901 | 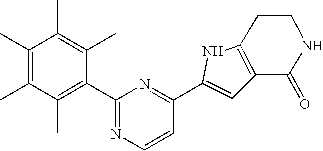 | 2-[2-(pentamethylphenyl)pyrimidin-4-yl]-1,5,6,7-tetrahydro- 4H-pyrrolo[3,2-c]pyridin-4-one |
| 902 | 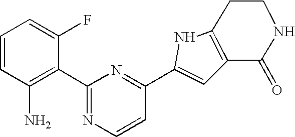 | 2-[2-(2-amino-6-fluorophenyl)pyrimidin-4-yl]-1,5,6,7- tetrahydro-4H-pyrrolo[3,2-c]pyridin-4-one |
| 903 | 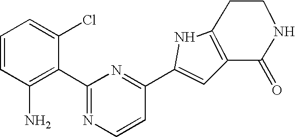 | 2-[2-(2-amino-6-chlorophenyl)pyrimidin-4-yl]-1,5,6,7- tetrahydro-4H-pyrrolo[3,2-c]pyridin-4-one |
| 904 | 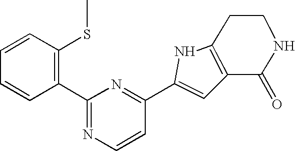 | 2-{2-[2-(methylthio)phenyl]pyrimidin-4-yl]56 -1,5,6,7- tetrahydro-4H-pyrrolo[3,2-c]pyridin-4-one |
| 905 | 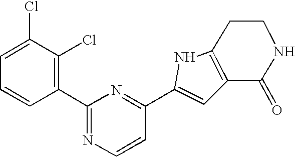 | 2-[2-(2,3-dichlorophenyl)pyrimidin-4-yl]-1,5,6,7-tetrahydro- 4H-pyrrolo[3,2-c]pyridin-4-one |
| 906 | 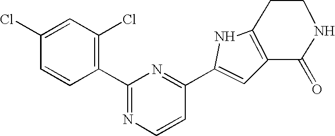 | 2-[2-(2,4-dichlorophenyl)pyrimidin-4-yl]-1,5,6,7-tetrahydro- 4H-pyrrolo[3,2-c]pyridin-4-one |
| 907 | 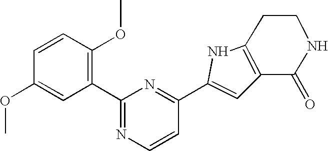 | 2-[2-(2,5-dimethoxyphenyl)pyrimidin-4-yl]-1,5,6,7- tetrahydro-4H-pyrrolo[3,2-c]pyridin-4-one |
| 908 | 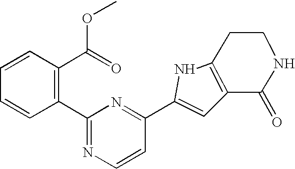 | methyl 2-[4-(4-oxo-4,5,6,7-tetrahydro-1H-pyrrolo[3,2- c]pyridin-2-yl)pyrimidin-2-yl]benzoate |
| 909 | 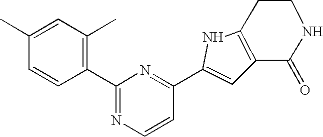 | 2-[2-(2,4-dimethylphenyl)pyrimidin-4-yl]-1,5,6,7-tetrahydro- 4H-pyrrolo[3,2-c]pyridin-4-one |
| 910 | 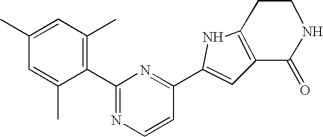 | 2-(2-mesitylpyrimidin-4-yl)-1,5,6,7-tetrahydro-4H- pyrrolo[3,2-c]pyridin-4-one |
| 911 | 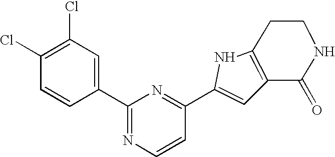 | 2-[2-(3,4-dichlorophenyl)pyrimidin-4-yl]-1,5,6,7-tetrahydro- 4H-pyrrolo[3,2-c]pyridin-4-one |
| 912 | 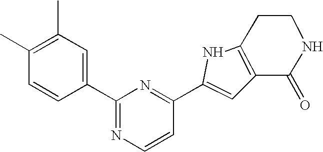 | 2-[2-(3,4-dimethylphenyl)pyrimidin-4-yl]-1,5,6,7-tetrahydro- 4H-pyrrolo[3,2-c]pyridin-4-one |
| 913 | 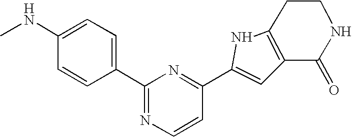 | 2-{2-[4-(methylamino)phenyl]pyrimidin.4-yl]56 -1,5,6,7- tetrahydro-4H-pyrrolo[3,2-c]pyridin-4-one |
| 914 | 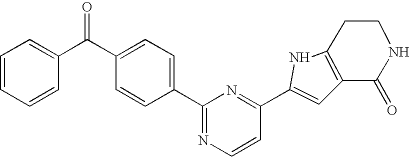 | 2-[2-(4-benzoylphenyl)pyrimidin-4-yl]-1,5,6,7-tetrahydro- 4H-pyrrolo[3,2-c]pyridin-4-one |
| 915 | 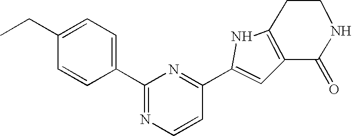 | 2-[2-(4-ethylphenyl)pyrimidin-4-yl]-1,5,6,7-tetrahydro-4H- pyrrolo[3,2-c]pyridin-4-one |
| 916 | 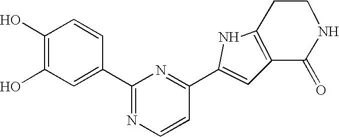 | 2-[2-(3,4-dihydroxyphenyl)pyrimidin-4-yl]-1,5,6,7- tetrahydro-4H-pyrrolo[3,2-c]pyridin-4-one |
| 917 | 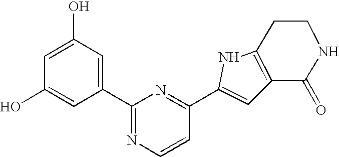 | 2-[2-(3,5-dihydroxyphenyl)pyrimidin-4-yl]-1 5,6,7- tetrahydro-4H-pyrro~o[3,2-c]pyridin-4-one |
| 918 | 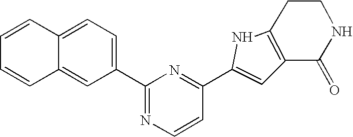 | 2-[2-(2-naphthyl)pyrimidin-4-yl]-1,5,6,7-tetrahydro-4H- pyrrolo[3,2-c]pyridin-4-one |
| 919 | 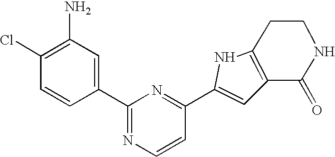 | 2-[2-(3-amino-4-chlorophenyl)pyrimidin-4-yl]-1,5,6,7- tetrahydro-4H-pyrrolo[3,2-c]pyridin-4-one |
| 920 | 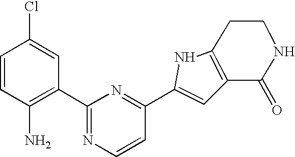 | 2-[2-(2-amino-5-chiorophenyl)pyrimidin-4-yl]-1,5,6,7- tetrahydro-4H-pyrrolo[3,2-c]pyridin-4-one |
| 921 | 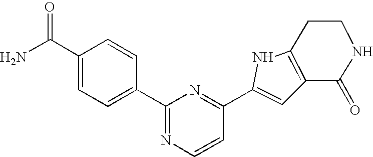 | 4-[4-(4-oxo-4,5,6,7-tetrahydro-1H-pyrrolo[3,2-c]pyridin-2- yl)pyrimidin-2-yl]benzamide |
| 922 | 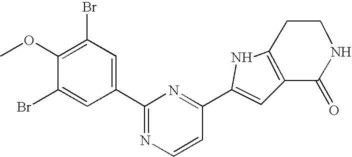 | 2-[2-(3,5-dibromo-4-methoxyphenyl)pyrimidin-4-yl]- 1,5,6,7-tetrahydro-4H-pyrrolo[3,2-c]pyridin-4-one |
| 923 | 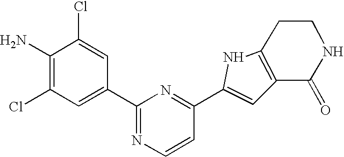 | 2-[2-(4-amino-3,5-dichlorophenyl)pyrimidin-4-yl]-1,5,6,7- tetrahydro-4H-pyrrolo[3,2-c]pyridin-4-one |
| 924 | 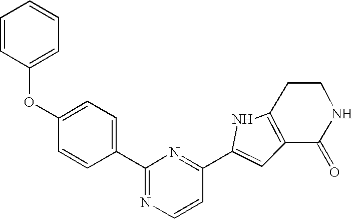 | 2-[2-(4-phenoxyphenyl)pyrimidin-4-yl]-1,5,6,7-tetrahydro- 4H-pyrrolo[3,2-c]pyridin-4-one |
| 925 | 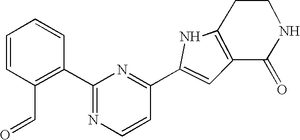 | 2-[4-(4-oxo-4,5,6,7-tetrahydro-1H-pyrrolo[3,2-c]pyridin-2- yl)pyrimidin-2-yl]benzaldehyde |
| 926 | 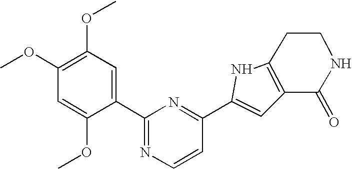 | 2-[2-(2,4,5-trimethoxyphenyl)pyrimidin-4-yl]-1,5,6,7- tetrahydro-4H-pyrrolo[3,2-c]pyridin-4-one |
| 927 | 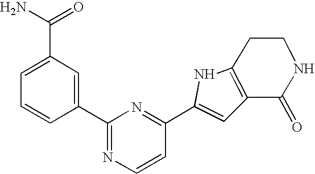 | 3-[4-(4-oxo-4,5,6,7-tetrahydro-1H-pyrrolo[3,2-c]pyridin-2- yl)pyrimidin-2-yl]benzamide |
| 928 | 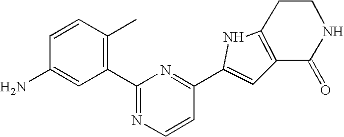 | 2-[2-(5-amino-2-methylphenyl)pyrimidin-4-yl]-1,5,6,7- tetrahydro-4H-pyrrolo[3,2-c]pyridin-4-one |
| 929 | 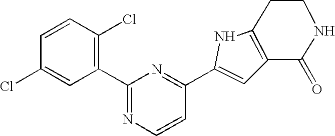 | 2-[2-(2,5-dichlorophenyl)pyrimidin-4-yl]-1,5,6,7-tetrahydro- 4H-pyrrolo[3,2-c]pyridin-4-one |
| 930 | 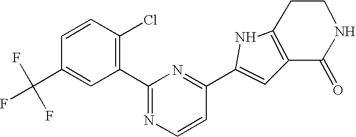 | 2-(2-[2-chloro-5-(trifluoromethyl)phenyl]pyrimidin-4-yl]- 1,5,6,7-tetrahydro-4H-pyrrolo[3,2-c]pyridin-4-one |
| 931 | 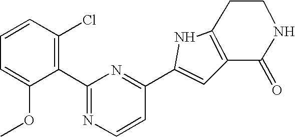 | 2-[2-(2-chloro-6-methoxyphenyl)pyrimidin-4-yl]-1,5,6,7- tetrahydro-4H-pyrrolo[3,2-c]pyridin-4-one |
| 932 | 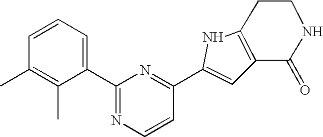 | 2-[2-(2,3-dimethylphenyl)pyrimidin-4-yl]-1,5,6,7-tetrahydro- 4H-pyrrolo[3,2-c]pyridin-4-one |
| 933 | 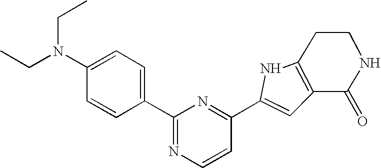 | 2-{2-[4-(diethylamino)phenyl]pyrimidin-4-yl]56 -1,5,6,7- tetrahydro-4H-pyrrolof3,2-c]pyridin-4-one |
| 934 | 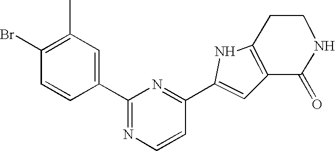 | 2-[2-(4-bromo-3-methylphenyl)pyrimidin-4-yl]-1,5,6,7- tetrahydro-4H-pyrrolo[3,2-c]pyridin-4-one |
| 935 | 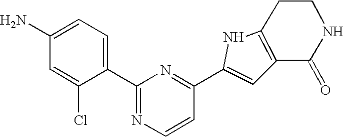 | 2-[2-(4-amino-2-chlorophenyl)pyrimidin-4-yl]-1,5,6,7- tetrahydro-4H-pyrrolo(3,2-c]pyridin-4-one |
| 936 | 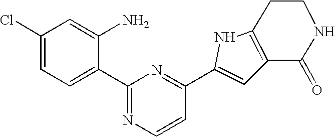 | 2-[2-(2-amino-4-chlorophenyl)pyrimidin-4-yl]-1,5,6,7- tetrahydro-4H-pyrrolo[3,2-c]pyridin-4-one |
| 937 | 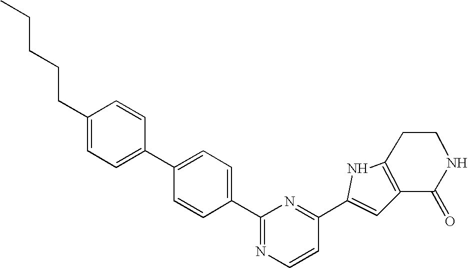 | 2-[2-(4′-pentyl-1,1′-biphenyl-4-yl)pyrimidin-4-yl]-1,5,6,7- tetrahydro-4H-pyrrolo[3,2-c]pyridin-4-one |
| 938 | 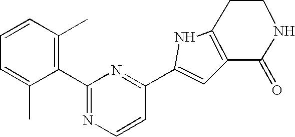 | 2-[2-(2,6-dimethylphenyl)pyrimidin-4-yl]-1,5,6,7-tetrahydro- 4H-pyrrolo[3,2-c]pyridin-4-one |
| 939 | 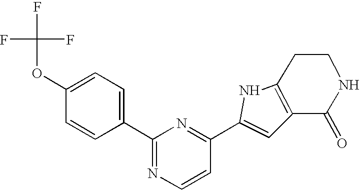 | 2-{2-[4-(trifluoromethoxy)phenyl]pyrimidin-4-yl}-1,5,6,7- tetrahydro-4H-pyrrolo[3,2-c]pyridin-4-one |
| 940 | 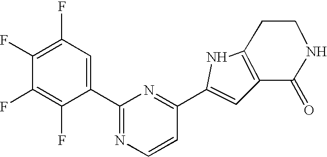 | 2-[2-(2,3,4,5-tetrafluorophenyl)pyrimidin-4-yl]-1,5,6,7- tetrahydro-4H-pyrrolo[3,2-c]pyridin-4-one |
| 941 | 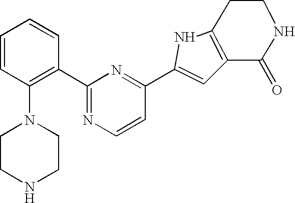 | 2-[2-(2-piperazin-1-ylphenyl)pyrimidin-4-yl]-1,5,6,7- tetrahydro-4H-pyrrolo[3,2-c]pyridin-4-one |
| 942 | 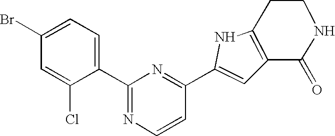 | 2-[2-(4-bromo-2-chlorophenyl)pyrimidin-4-yl]-1,5,6,7- tetrahydro-4H-pyrrolo[3,2-c]pyridin-4-one |
| 943 | 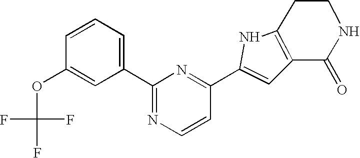 | 2-{2-[3-(trifluoromethoxy)phenyl]pyrimidin-4-yl]56 -1,5,6,7- tetrahydro-4H-pyrrolo[3,2-c]pyridin-4-one |
| 944 | 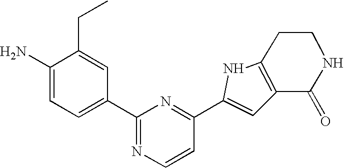 | 2-[2-(4-amino-3-ethylphenyl)pyrimidin-4-yl]-1,5,6,7- tetrahydro-4H-pyrrolo[3,2-c]pyridin-4-one |
| 945 | 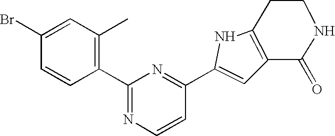 | 2-[2-(4-bromo-2-methylphenyl)pyrimidin-4-yl]-1,5,6,7- tetrahydro-4H-pyrrolo[3,2-c]pyridin-4-one |
| 946 | 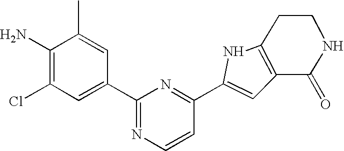 | 2-[2-(4-amino-3-chloro-5-methylphenyl)pyrimidin-4-yl]- 1,5,6,7-tetrahydro-4H-pyrrolo[3,2-c]pyridin-4-one |
| 947 | 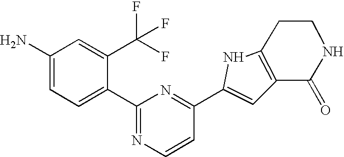 | 2-{2-[4-amino-2-(trifluoromethyl)phenyl]pyrimidin-4-yl]- 1,5,6,7-tetrahydro-4H-pyrrolo[3,2-c]pyridin-4-one |
| 948 | 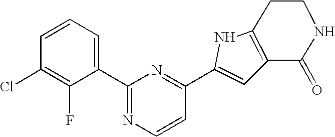 | 2-[2-(3-chloro-2-fluorophenyl)pyrimidin-4-yl]-1,5,6,7- tetrahydro-4H-pyrrolo[3,2-c]pyridin-4-one |
| 949 | 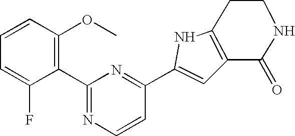 | 2-[2-(2-fluoro-6-methoxyphenyl)pyrimidin-4-yl]-1,5,6,7- tetrahydro-4H-pyrrolo[3,2-c]pyridin-4-one |
| 950 | 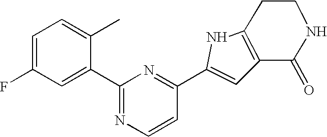 | 2-[2-(5-fluoro-2-methylphenyl)pyrimidin-4-yl]-1,5,6,7- tetrahydro-4H-pyrrolo[3,2-c]pyridin-4-one |
| 951 | 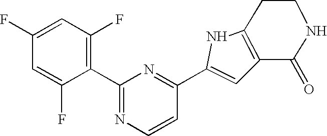 | 2-[2-(2,4,6-trifluorophenyl)pyrimidin-4-yl]-1,5,6,7- tetrahydro-4H-pyrrolo[3,2-c]pyridin-4-one |
| 952 | 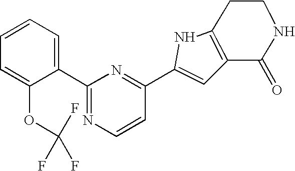 | 2-{2-[2-(trifluoromethoxy)phenyl]pyrimidin-4-yl]56 -1,5,6,7- tetrahydro-4H-pyrrolo[3,2-c]pyridin-4-one |
| 953 | 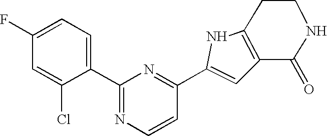 | 2-[2-(2-chloro-4-fluorophenyl)pyrimidin-4-yl]-1,5,6,7- tetrahydro-4H-pyrrolor3,2-c]pyridin-4-one |
| 954 | 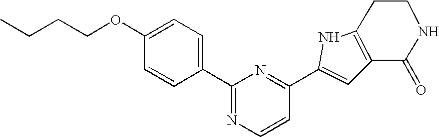 | 2-[2-(4-butoxyphenyl)pyrimidin-4-yl]-1,5,6,7-tetrahydro-4H- pyrrolo[3,2-c]pyridin-4-one |
| 955 | 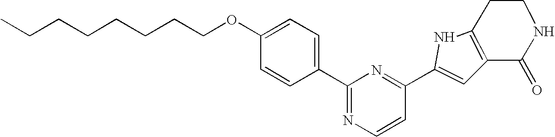 | 2-{2-[4-(octyloxy)phenyl]pyrimidin-4-yl]56 -1,5,6,7-tetrahydro- 4H-pyrrolo[3,2-c]pyridin-4-one |
| 956 | 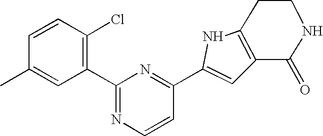 | 2-[2-(2-chloro-5-methylphenyl)pyrimidin-4-yl]-1,5,6,7- tetrahydro-4H-pyrrolo[3,2-c]pyridin-4-one |
| 957 | 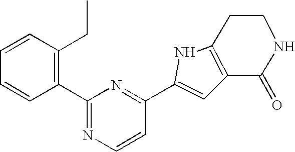 | 2-[2-(2-ethylphenyl)pyrimidin-4-yl]-1,5,6,7-tetrahydro-4H- pyrrolo[3,2-c]pyridin-4-one |
| 958 | 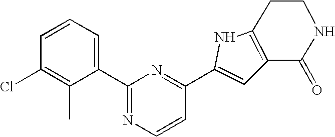 | 2-[2-(3-chloro-2-methylphenyl)pyrimidin-4-yl]-1,5,6,7- tetrahydro-4H-pyrroloF3,2-c]pyridin-4-one |
| 959 | 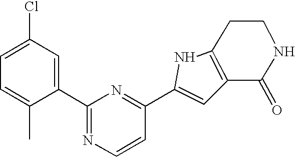 | 2-[2-(5-chloro-2-methylphenyl)pyrimidin-4-yl]-1,5,6,7- tetrahydro-4H-pyrrolo[3,2-c]pyridin-4-one |
| 960 | 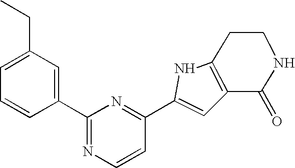 | 2-[2-(3-ethylphenyl)pyrimidin-4-yl]-1,5,6,7-tetrahydro-4H- pyrrolo[3,2-c]pyridin-4-one |
| 961 | 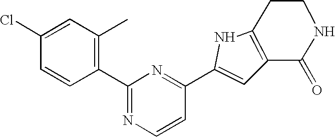 | 2-[2-(4-chloro-2-methylphenyl)pyrimidin-4-yl]-1,5,6,7- tetrahydro-4H-pyrrolo[3,2-c]pyridin-4-one |
| 962 | 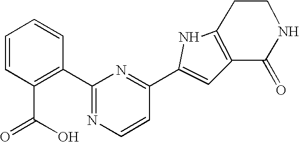 | 2-[4-(4-oxo-4,5,6,7-tetrahydro-1H-pyrrolo[3,2-c]pyridin-2- yl)pyrimidin-2-yl]benzoic acid |
| 963 | 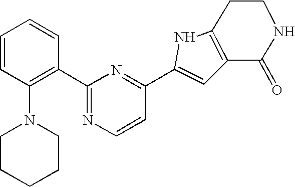 | 2-[2-(2-piperidin-1-ylphenyl)pyrimidin-4-yl]-1,5,6,7- tetrahydro-4H-pyrrolo[3,2-c]pyridin-4-one |
| 964 | 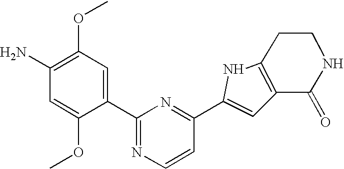 | 2-[2-(4-amino-2,5-dimethoxyphenyl)pyrimidin-4-yl]-1,5,6,7- tetrahydro-4H-pyrrolo[3,2-c]pyridin-4-one |
| 965 | 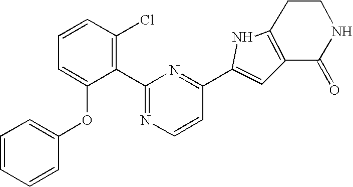 | 2-[2-(2-chloro-6-phenoxyphenyl)pyrimidin-4-yl]-1,5,6,7- tetrahydro-4H-pyrrolo[3,2-c]pyridin-4-one |
| 966 | 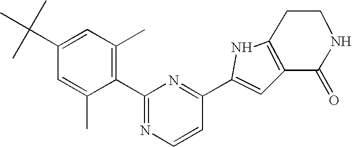 | 2-[2-(4-tert-butyl-2,6-dimethylphenyl)pyrimidin-4-yl]- 1,5,6,7-tetrahydro-4H-pyrrolo[3,2-c]pyridin-4-one |
| 967 | 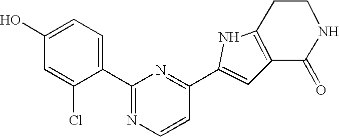 | 2-[2-(2-chloro-4-hydroxyphenyl)pyrimidin-4-yl]-1,5,6,7- tetrahydro-4H-pyrrolof3,2-c]pyridin-4-one |
| 968 | 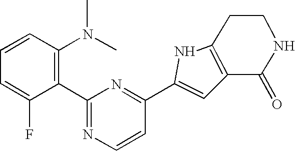 | 2-{2-[2-(dimethylamino)-6-fluorophenyl]pyrimidin-4-yl]56 - 1,5,6,7-tetrahydro-4H-pyrrolo[3,2-c]pyridin-4-one |
| 969 | 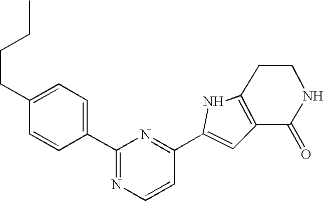 | 2-[2-(4-butylphenyl)pyrimidin-4-yl]-1,5,6,7-tetrahydro-4H- pyrrolo[3,2-c]pyridin-4-one |
| 970 | 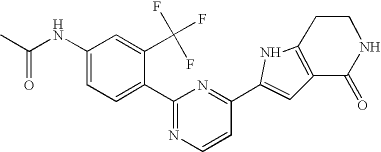 | N-[4-[4-(4-oxo-4,5,6,7-tetrahydro-1H-pyrrolo[3,2-c]pyridin- 2-yl)pyrimidin-2-yl]-3-(trifluoromethyl)phenyl]acetamide |
| 971 | 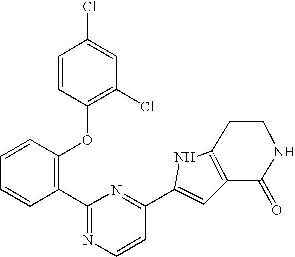 | 2-{2-[2-(2,4-dichlorophenoxy)phenyl]pyrimidin-4-yl}- 1,5,6,7-tetrahydro-4H-pyrrolo[3,2-c]pyridin-4-one |
| 972 | 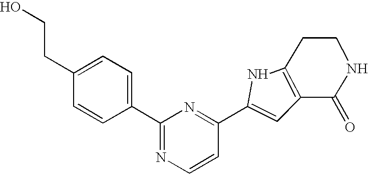 | 2-{2-[4-(2-hydroxyethyl)phenyl]pyrimidin-4-yl]56 -1,5,6,7- tetrahydro-4H-pyrrolo[3,2-c]pyridin-4-one |
| 973 | 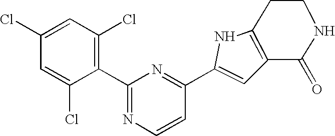 | 2-[2-(2,4,6-trichlorophenyl)pyrimidin-4-yl]-1,5,6,7- tetrahydro-4H-pyrrolo[3,2-c]pyridin-4-one |
| 974 | 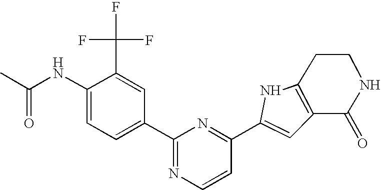 | N-[4-[4-(4-oxo-4,5,6,7-tetrahydro-1H-pyrrolo[3,2-c]pyridin- 2-yl)pyrimidin-2-yl]-2-(trifluoromethyl)phenyl]acetamide |
| 975 | 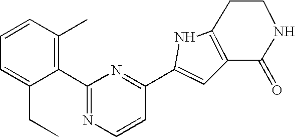 | 2-[2-(2-ethyl-6-methylphenyl)pyrimidin-4-yl]-1,5,6,7- tetrahydro-4H-pyrrolo[3,2-c]pyridin-4-one |
| 976 | 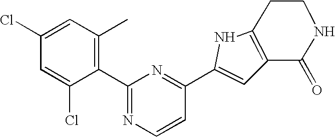 | 2-[2-(2,4-dichloro-6-methylphenyl)pyrimidin-4-yl]-1,5,6,7- tetrahydro-4H-pyrrolo[3,2-c]pyridin-4-one |
| 977 | 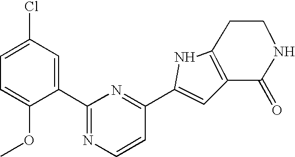 | 2-[2-(5-chloro-2-methoxyphenyl)pyrimidin-4-yl]-1,5,6,7- tetrahydro-4H-pyrrolo[3,2-c]pyridin-4-one |
| 978 | 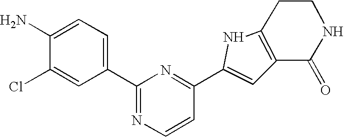 | 2-[2-(4-amino-3-chlorophenyl)pyrimidin-4-yl]-1,5,6,7- tetrahydro-4H-pyrrolo[3,2-c]pyridin-4-one |
| 979 | 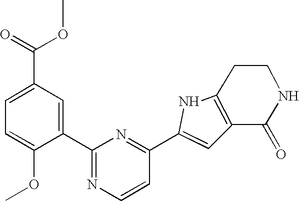 | methyl 4-methoxy-3-[4-(4-oxo-4,5,6,7-tetrahydro-1H- pyrrolo[3,2-c]pyridin-2-yl)pyrimidin-2-yl]benzoate |
| 980 | 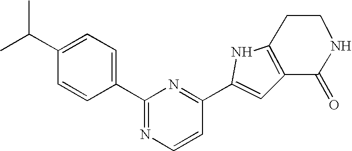 | 2-[2-(4-isopropylphenyl)pyrimidin-4-yl]-1,5,6,7-tetrahydro- 4H-pyrrolo[3,2-c]pyridin-4-one |
| 981 | 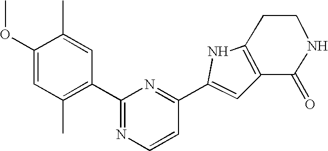 | 2-[2-(4-methoxy-2,5-dimethylphenyl)pyrimidin-4-yl]- 1,5,6,7-tetrahydro-4H-pyrrolo[3,2-c]pyridin-4-one |
| 982 | 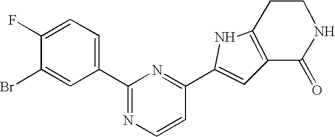 | 2-[2-(3-bromo-4-fluorophenyl)pyrimidin-4-yl]-1,5,6,7- tetrahydro-4H-pyrrolo[3,2-c]pyridin-4-one |
| 983 | 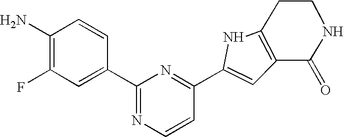 | 2-[2-(4-amino-3-fluorophenyl)pyrimidin-4-yl]-1,5,6,7- tetrahydro-4H-pyrrolo[3,2-c]pyridin-4-one |
| 984 | 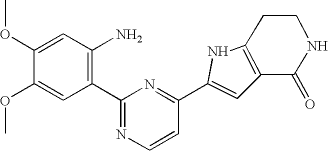 | 2-[2-(2-amino-4,5-dimethoxyphenyl)pyrimidin-4-yl]-1,5,6,7- tetrahydro-4H-pyrrolo[3,2-c]pyridin-4-one |
| 985 | 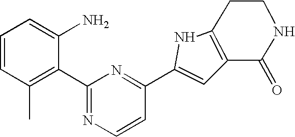 | 2-[2-(2-amino-6-methylphenyl)pyrimidin-4-yl]-1,5,6,7- tetrahydro-4H-pyrrolo[3,2-c]pyridin-4-one |
| 986 | 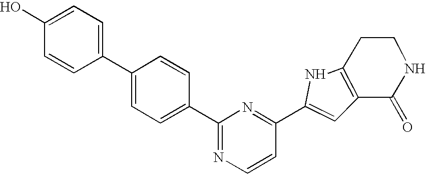 | 2-[2-(4-hydroxy-1,1-biphenyl-4-yl)pyrimidin-4-yl]-1,5,6,7- tetrahydro-4H-pyrrolo[3,2-c]pyridin-4-one |
| 987 | 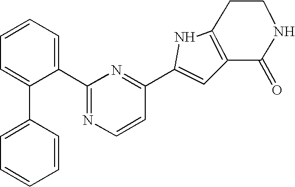 | 2-[2-(1,1′-biphenyl-2-yl)pyrimidin-4-yl]-1,5,6,7-tetrahydro- 4H-pyrrolo[3,2-c]pyridin-4-one |
| 988 | 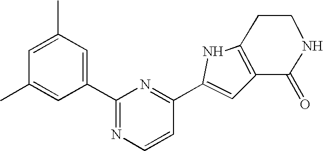 | 2-[2-(3,5-dimethylphenyl)pyrimidin-4-yl]-1,5,6,7-tetrahydro- 4H-pyrrolo[3,2-c]pyridin-4-one |
| 989 | 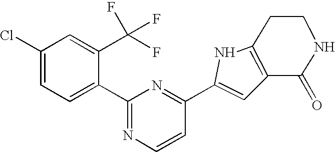 | 2-{2-[4-chloro-2-(trifluoromethyl)phenyl]pyrimidin-4-yl]- 1,5,6,7-tetrahydro-4H-pyrrolo[3,2-c]pyridin-4-one |
| 990 | 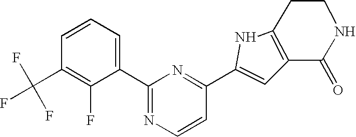 | 2-{2-[2-fluoro-3-(trifluoromethyl)phenyl]pyrimidin-4-yl}- 1,5,6,7-tetrahydro-4H-pyrrolo[3,2-c]pyridin-4-one |
| 991 | 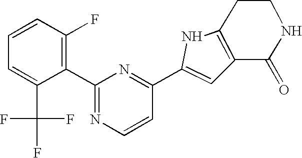 | 2-{2-[2-fluoro-6-(trifluoromethyl)phenyl]pyrimidin-4-yl}- 1,5,6,7-tetrahydro-4H-pyrrolo[3,2-c]pyridin-4-one |
| 992 | 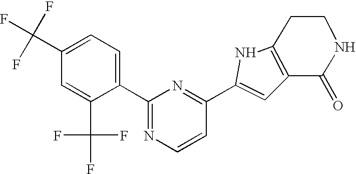 | 2-{2-[2,4-bis(trifluoromethyl)phenyl]pyrimidin-4-yl}-1,5,6,7- tetrahydro-4H-pyrrolo[3,2-c]pyridin-4-one |
| 993 | 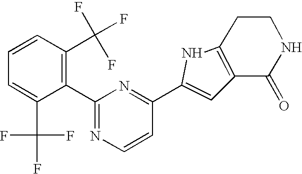 | 2-{2-[2,6-bis(trifluoromethylphenyl]pyrimidin-4-yl}-1,5,6,7- tetrahydro-4H-pyrrolo[3,2-c]pyridin-4-one |
| 994 | 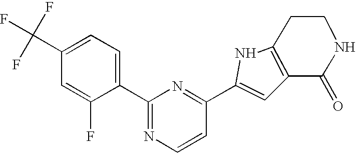 | 2-{2-[2-fluoro-4-(trifluoromethyl)phenyl]pyrimidin-4-yl}- 1,5,6,7-tetrahydro-4H-pyrrolo[3,2-c]pyridin-4-one |
| 995 | 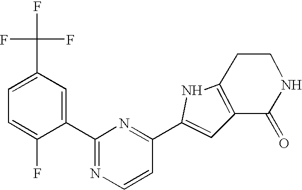 | 2-{2-[2-fluoro-5-(trifluoromethyl)phenyl]pyrimidin-4-yl}- 1,5,6,7-tetrahydro-4H-pyrrolo[3,2-c]pyridin-4-one |
| 996 | 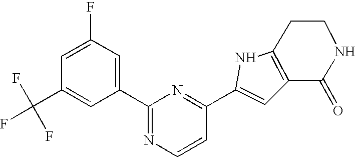 | 2-{2-[3-fluoro-5-(trifluoromethyl)phenyl]pyrimidin-4-yl}- 1,5,6,7-tetrahydro-4H-pyrrolo[3,2-c]pyridin-4-one |
| 997 | 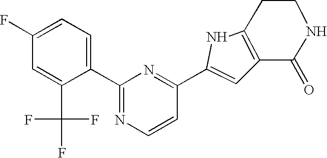 | 2-{2-[4-fluoro-2-(trifluoromethyl)phenyl]pyrimidin-4-yl}- 1,5,6,7-tetrahydro-4H-pyrrolo[3,2-c]pyridin-4-one |
| 998 | 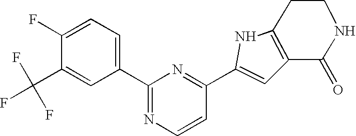 | 2-{2-[4-fluoro-3-(trifluoromethyl)phenyl]pyrimidin-4-yl}- 1,5,6,7-tetrahydro-4H-pyrrolo[3,2-c]pyridin-4-one |
| 999 | 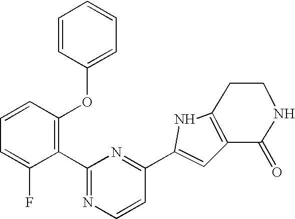 | 2-[2-(2-fluoro-6-phenoxyphenyl)pyrimidin-4-yl]-1,5,6,7- tetrahydro-4H-pyrrolo[3,2-c]pyridin-4-one |
| 1000 | 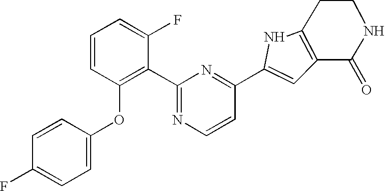 | 2-{2-[2-fluoro-6-(4-fluorophenoxy)phenyl]pyrimidin-4-yl}- 1,5,6,7-tetrahydro-4H-pyrrolo[3,2-c]pyridin-4-one |
| 1001 | 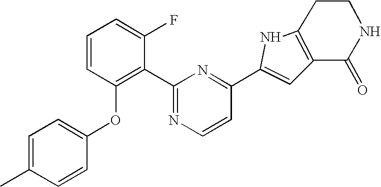 | 2-{2-[2-fluoro-6-(4-methylphenoxy)phenyl]pyrimidin-4-yl}- 1,5,6,7-tetrahydro-4H-pyrrolo[3,2-c]pyridin-4-one |
| 1002 | 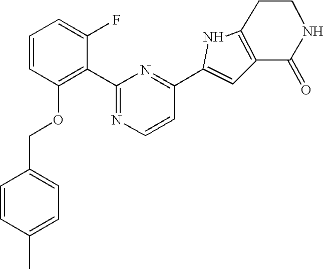 | 2-(2-{2-fluoro-6-[(4-methylbenzyl)oxylphenyl}pyrimidin-4- yl)-1,5,6,7-tetrahydro-4H-pyrrolo[3,2-c]pyridin-4-one |
| 1003 | 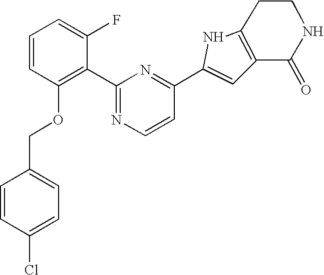 | 2-(2-{2-[(4-chlorobenzyl)oxy]-6-fluorophenyl}pyrimidin-4- yl)-1,5,6,7-tetrahydro-4H-pyrrolo[3,2-c]pyridin-4-one |
| 1004 | 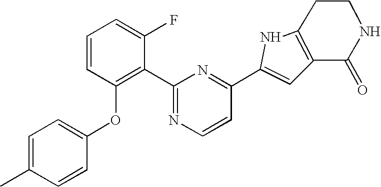 | 2-(2-{2-fluoro-6-[(4-methylphenyl)thio]phenyl}pyrimidin-4- yl)-1,5,6,7-tetrahydro-4H-pyrrolo[3,2-c]pyridin-4-one |
| 1005 | 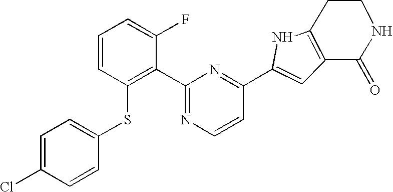 | 2-(2-{2-[(4-chlorophenyl)thio]-6-fluorophenyl}pyrimidin-4- yl)-1,5,6,7-tetrahydro-4H-pyrrolo[3,2-c]pyridin-4-one |
| 1006 | 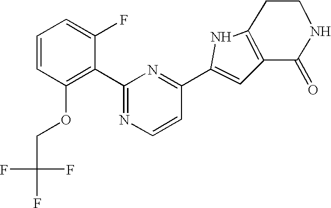 | 2-{2-[2-fluoro-6-(2,2,2-trifluoroethoxy)phenyl]pyrimidin-4- yl}-1,5,6,7-tetrahydro-4H-pyrrolo[3,2-c]pyridin-4-one |
| 1007 | 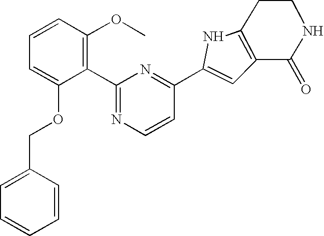 | 2-{2-[2-(benzyloxy)-6-methoxyphenyl]pyrimidin-4-yl}- 1,5,6,7-tetrahydro-4H-pyrrolo[3,2-c]pyridin-4-one |
| 1008 | 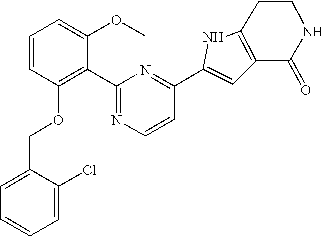 | 2-(2-{2-[(2-chlorobenzyl)oxy]-6-methoxyphenyl}pyrimidin- 4-yl)-1,5,6,7-tetrahydro-4H-pyrrolo[3,2-c]pyridin-4-one |
| 1009 | 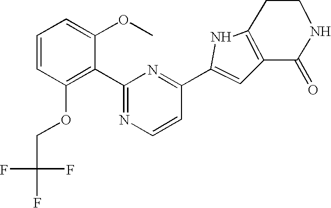 | 2-{2-[2-methoxy-6-(2,2,2-trifluoroethoxy)phenyl]pyrimidin- 4-yl}-1,5,6,7-tetrahydro-4H-pyrrolo[3,2-c]pyridin-4-one |
| 1010 | 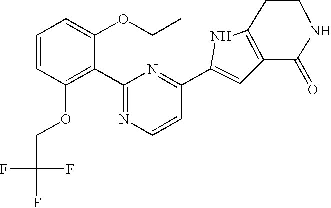 | 2-}2-[2-ethoxy-6-(2,2,2-trifluoroethoxy)phenyl]pyrimidin-4- yl}-1,5,6,7-tetrahydro-4H-pyrrolo[3,2-c]pyridin-4-one |
| 1011 | 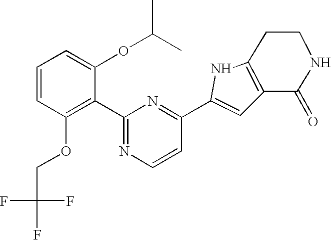 | 2-{2-[2-isopropoxy-6-(2,2,2- trifluoroethoxy)phenyl]pyrimidin-4-yl}-1,5,6,7-tetrahydro- 4H-pyrrolo[3,2-c]pyridin-4-one |
| 1012 | 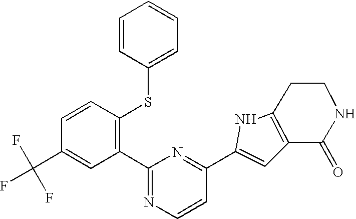 | 2-{2-[2-(phenylthio)-5-(trifluoromethyl)phenyl]pyrimidin-4- yl}-1,5,6,7-tetrahydro-4H-pyrrolo[3,2-c]pyridin-4-one |
| 1013 | 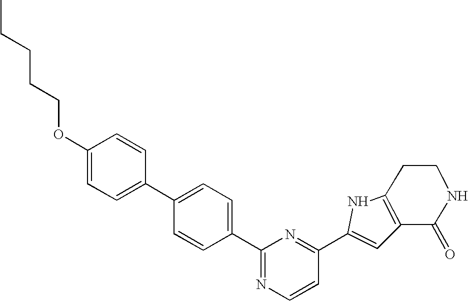 | 2-{2-[4-(pentyloxy)-1,1′-biphenyl-4-yl]pyrimidin-4-yl}- 1,5,6,7-tetrahydro-4H-pyrrolo[3,2-c]pyridin-4-one |
| 1014 | 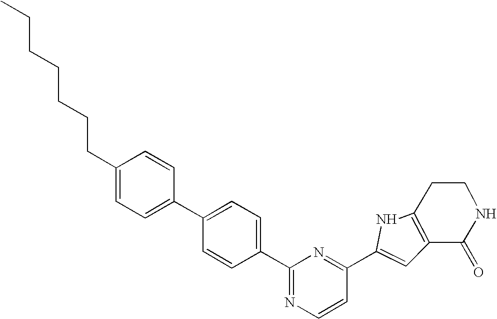 | 2-[2-(4′-heptyl-1,1′-biphenyl-4-yl)pyrimidin-4-yl]-1,5,6,7- tetrahydro-4H-pyrrolo[3,2-c]pyridin-4-one |
| 1015 | 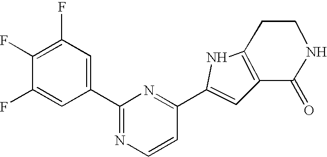 | 2-[2-(3,4,5-trifluorophenyl)pyrimidin-4-yl]-1,5,6,7- tetrahydro-4H-pyrrolo[3,2-c]pyridin-4-one |
| 1016 | 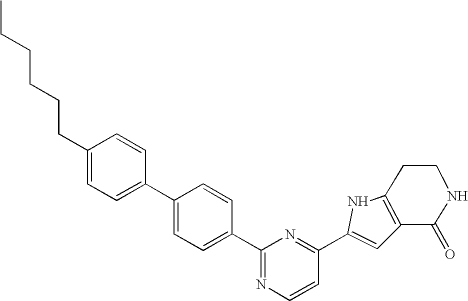 | 2-[2-(4′-hexyl-1,1′-biphenyl-4-yl)pyrimidin-4-yl]-1,5,6,7- tetrahydro-4H-pyrrolo[3,2-c]pyridin-4-one |
| 1017 | 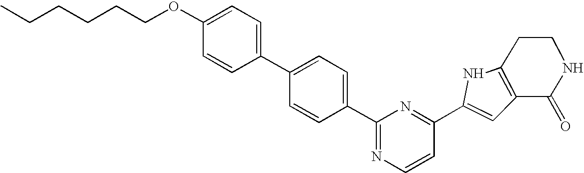 | 2-{2-[4′-(octyloxy)-1,1′-biphenyl-4-yl]pyrimidin-4-yl}-1,5,6,7- tetrahydro-4H-pyrrolo[3,2-c]pyridin-4-one |
| 1018 | 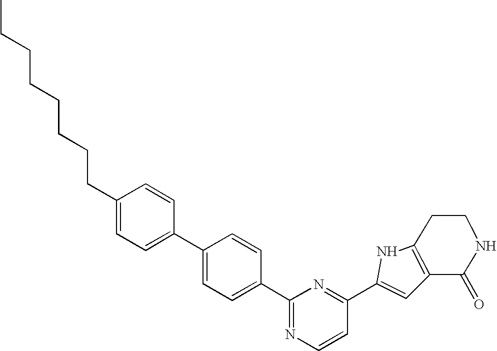 | 2-[2-(4-octyl-1,1′-biphenyl-4-yl)pyrimidin-4-yl]-1,5,6,7- tetrahydro-4H-pyrrolo[3,2-c]pyridin-4-one |
| 1019 | 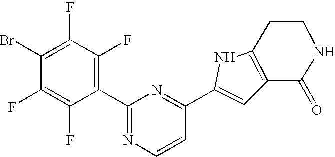 | 2-[2-(4-bromo-2,3,5,6-tetrafluorophenyl)pyrimidin-4-yl]- 1,5,6,7-tetrahydro-4H-pyrrolo[3,2-c]pyridin-4-one |
| 1020 | 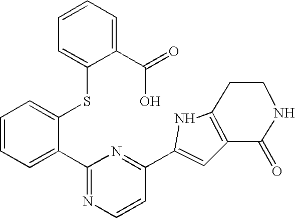 | 2-({2-[4-(4-oxo-4,5,6,7-tetrahydro-1H-pyrrolo[3,2-c]pyridin- 2-yl)pyrimidin-2-yl]phenyl}thio)benzoic acid |
| 1021 | 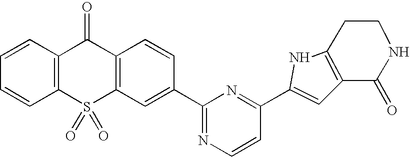 | 2-[2-(10,10-dioxido-9-oxo-9H-thioxanthen-3-yl)pyrimidin-4- yl]-1,5,6,7-tetrahydro-4H-pyrrolo[3,2-c]pyridin-4-one |
| 1022 | 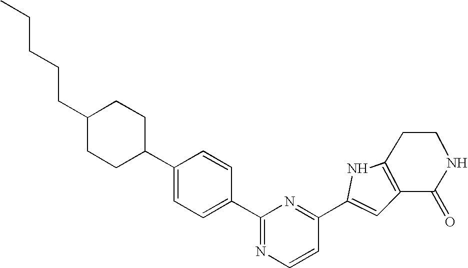 | 2-{2-[4-(4-pentylcyclohexyl)phenyl]pyrimidin-4-yl}-1,5,6,7- tetrahydro-4H-pyrrolo[3,2-c]pyridin-4-one |
| 1023 | 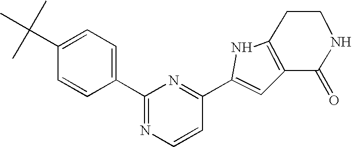 | 2-[2-(4-tert-butylphenyl)pyrimidin-4-yl]-1,5,6,7-tetrahydro- 4H-pyrrolo[3,2-c]pyridin-4-one |
| 1024 | 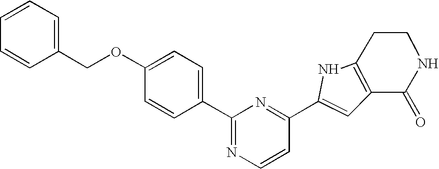 | 2-{2-[4-(benzyloxy)phenyl]pyrimidin-4-yl}-1,5,6,7- tetrahydro-4H-pyrrolo[3,2-c]pyridin-4-one |
| 1025 | 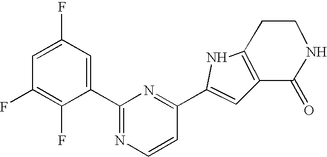 | 2-[2-(2,3,5-trifluorophenyl)pyrimidin-4-yl]-1,5,6,7- tetrahydro-4H-pyrrolo[3,2-c]pyridin-4-one |
| 1026 | 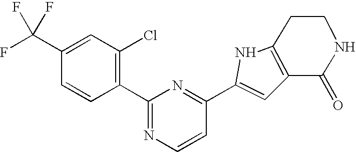 | 2-{2-[2-chloro-4-(trifluoromethyl)phenyl]pyrimidin-4-yl}- 1,5,6,7-tetrahydro-4H-pyrrolo[3,2-c]pyridin-4-one |
| 1027 | 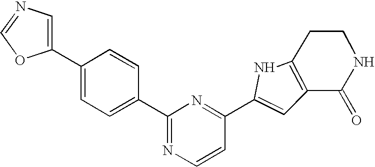 | 2-{2-[4-(1,3-oxazol-5-yl)phenyl]pyrimidin-4-yl}-1,5,6,7- tetrahydro-4H-pyrrolo[3,2-c]pyridin-4-one |
| 1028 | 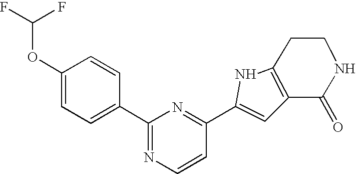 | 2-{2-[4-(difluoromethoxy)phenyl]pyrimidin-4-yl}-1,5,6,7- tetrahydro-4H-pyrrolo[3,2-c]pyridin-4-one |
| 1029 | 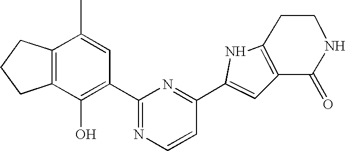 | 2-[2-(4-hydroxy-7-methyl-2,3-dihydro-1H-inden-5- yl)pyrimidin-4-yl]-1,5,6,7-tetrahydro-4H-pyrrolo[3,2- c]pyridin-4-one |
| 1030 | 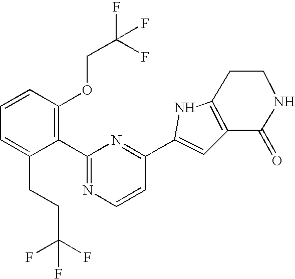 | 2-{2-[2,6-bis(2,2,2-trifluoroethoxy)phenyl]pyrimidin-4-yl}- 1,5,6,7-tetrahydro-4H-pyrrolo[3,2-c]pyridin-4-one |
| 1031 | 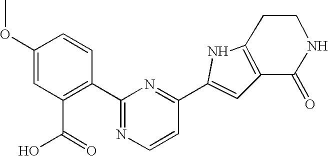 | 5-methoxy-2-[4-(4-oxo-4,5,6,7-tetrahydro-1H-pyrrolo[3,2- c]pyridin-2-yl)pyrimidin-2-yl]benzoic acid |
| 1032 | 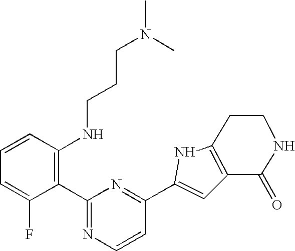 | 2-[2-(2-{[3-(dimethylamino)propyl]amino}-6- fluorophenyl)pyrimidin-4-yl]-1,5,6,7-tetrahydro-4H- pyrrolo[3,2-c]pyridin-4-one |
| 1033 | 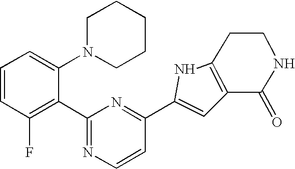 | 2-[2-(2-fluoro-6-piperidin-1-ylphenyl)pyrimidin-4-yl]- 1,5,6,7-tetrahydro-4H-pyrrolo[3,2-c]pyridin-4-one |
| 1034 | 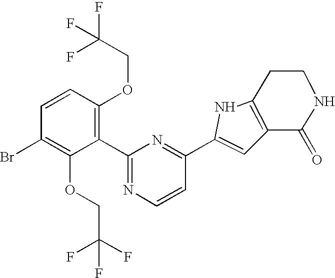 | 2-{2-[3-bromo-2,6-bis(2,2,2- trifluoroethoxy)phenyl]pyrimidin-4-yl}-1,5,6,7-tetrahydro- 4H-pyrrolo[3,2-c]pyridin-4-one |
| 1035 | 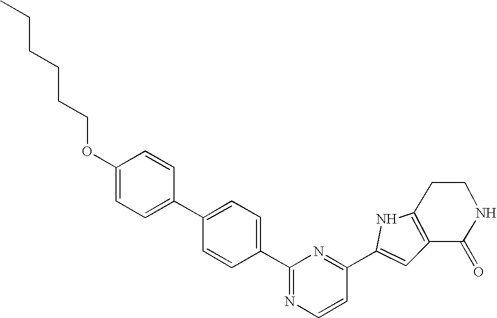 | 2-{2-[4′-(hexyloxy)-1,1′-biphenyl-4-yl]pyrimidin-4-yl}- 1,5,6,7-tetrahydro-4H-pyrrolo[3,2-c]pyridin-4-one |
| 1036 | 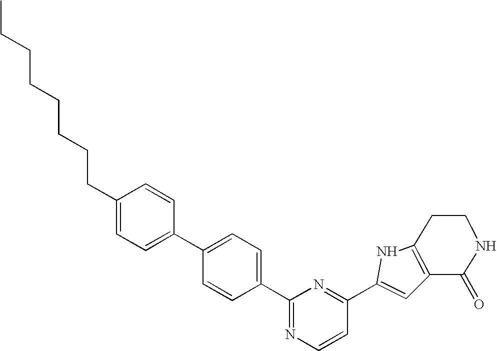 | 2-{2-[4′-(heptyloxy)-1,1′-biphenyl-4-yl]pyrimidin-4-yl}- 1,5,6,7-tetrahydro-4H-pyrrolo[3,2-c]pyridin-4-one |
| 1037 | 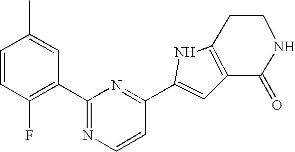 | 2-[2-(2-fluoro-5-methylphenyl)pyrimidin-4-yl]-1,5,6,7- tetrahydro-4H-pyrrolo[3,2-c]pyridin-4-one |
| 1038 | 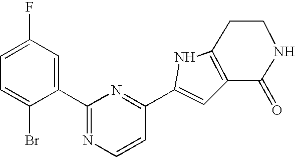 | 2-[2-(2-bromo-5-fluorophenyl)pyrimidin-4-yl]-1,5,6,7- tetrahydro-4H-pyrrolo[3,2-c]pyridin-4-one |
| 1039 | 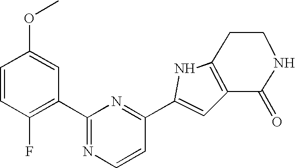 | 2-[2-(2-fluoro-5-methoxyphenyl)pyrimidin-4-yl]-1,5,6,7- tetrahydro-4H-pyrrolo[3,2-c]pyridin-4-one |
| 1040 | 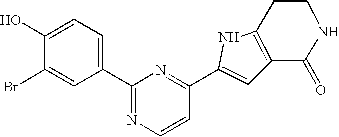 | 2-[2-(3-bromo-4-hydroxyphenyl)pyrimidin-4-yl]-1,5,6,7- tetrahydro-4H-pyrrolo[3,2-c]pyridin-4-one |
| 1041 | 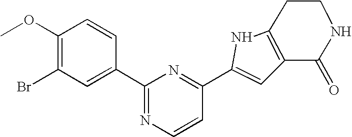 | 2-[2-(3-bromo-4-methoxyphenyl)pyrimidin-4-yl]-1,5,6,7- tetrahydro-4H-pyrrolo[3,2-c]pyridin-4-one |
| 1042 | 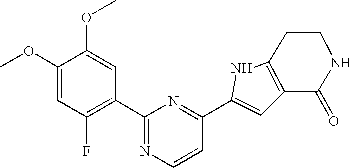 | 2-[2-(2-fluoro-4,5-dimethoxyphenyl)pyrimidin-4-yl]-1,5,6,7- tetrahydro-4H-pyrrolo[3,2-c]pyridin-4-one |
| 1043 | 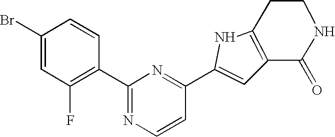 | 2-[2-(4-bromo-2-fluorophenyl)pyrimidin-4-yl]-1,5,6,7- tetrahydro-4H-pyrrolo[3,2-c]pyridin-4-one |
| 1044 | 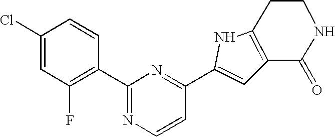 | 2-[2-(4-chloro-2-fluorophenyl)pyrimidin-4-yl]-1,5,6,7- tetrahydro-4H-pyrrolo[3,2-c]pyridin-4-one |
| 1045 | 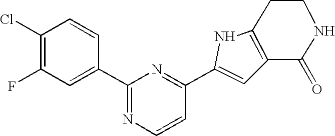 | 2-[2-(4-chloro-3-fluorophenyl)pyrimidin-4-yl]-1,5,6,7- tetrahydro-4H-pyrrolo[3,2-c]pyridin-4-one |
| 1046 | 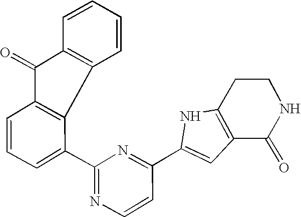 | 2-[2-(9-oxo-9H-fluoren-4-yl)pyrimidin-4-yl]-1,5,6,7- tetrahydro-4H-pyrrolo[3,2-c]pyridin-4-one |
| 1047 | 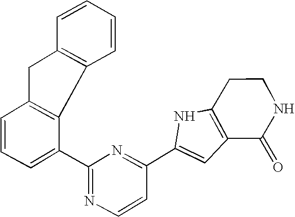 | 2-[2-(9H-fluoren-4-yl)pyrimidin-4-yl]-1,5,6,7-tetrahydro-4H- pyrrolo[3,2-c]pyridin-4-one |
| 1048 | 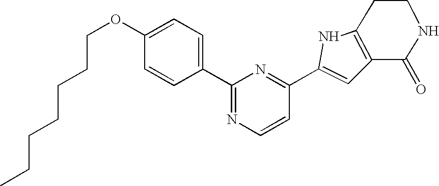 | 2-{2-[4-(heptyloxy)phenyl]pyrimidin-4-yl}-1,5,6,7- tetrahydro-4H-pyrrolo[3,2-c]pyridin-4-one |
| 1049 | 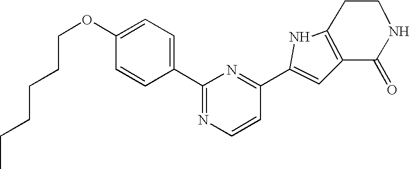 | 2-{2-[4-(hexyloxy)phenyl]pyrimidin-4-yl}-1,5,6,7-tetrahydro- 4H-pyrrolo[3,2-c]pyridin-4-one |
| 1050 | 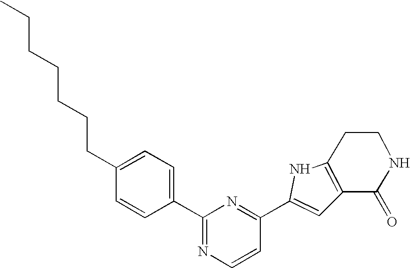 | 2-[2-(4-heptylphenyl)pyrimidin-4-yl]-1,5,6,7-tetrahydro-4H- pyrrolo[3,2-c]pyridin-4-one |
| 1051 | 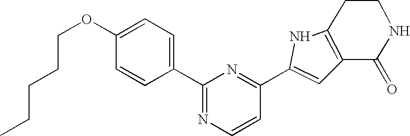 | 2-{2-[4-(pentyloxy)phenyl]pyrimidin-4-yl}-1,5,6,7- tetrahydro-4H-pyrrolo[3,2-c]pyridin-4-one |
| 1052 | 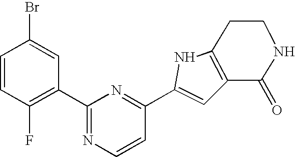 | 2-[2-(5-bromo-2-fluorophenyl)pyrimidin-4-yl]-1,5,6,7- tetrahydro-4H-pyrrolo[3,2-c]pyridin-4-one |
| 1053 | 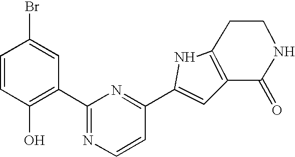 | 2-[2-(5-bromo-2-hydroxyphenyl)pyrimidin-4-yl]-1,5,6,7- tetrahydro-4H-pyrrolo[3,2-c]pyridin-4-one |
| 1054 | 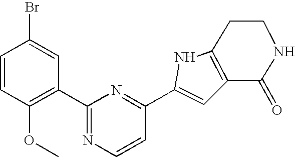 | 2-[2-(5-bromo-2-methoxyphenyl)pyrimidin-4-yl]-1,5,6,7- tetrahydro-4H-pyrrolo[3,2-c]pyridin-4-one |
| 1055 | 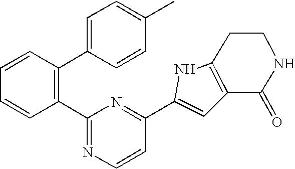 | 2-[2-(4-methyl-1,1′-biphenyl-2-yl)pyrimidin-4-yl]-1,5,6,7- tetrahydro-4H-pyrrolo[3,2-c]pyridin-4-one |
| 1056 | 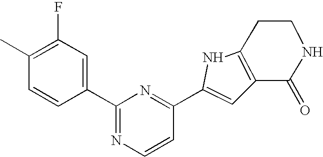 | 2-[2-(3-fluoro-4-methylphenyl)pyrimidin-4-yl]-1,5,6,7- tetrahydro-4H-pyrrolo[3,2-c]pyridin-4-one |
| 1057 | 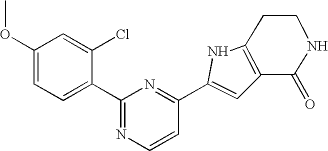 | 2-[2-(2-chloro-4-methoxyphenyl)pyrimidin-4-yl]-1,5,6,7- tetrahydro-4H-pyrrolo[3,2-c]pyridin-4-one |
| 1058 | 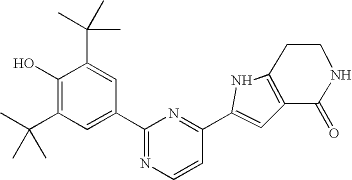 | 2-[2-(3,5-di-tert-butyl-4-hydroxyphenyl)pyrimidin-4-yl]- 1,5,6,7-tetrahydro-4H-pyrrolo[3,2-c]pyridin-4-one |
| 1059 | 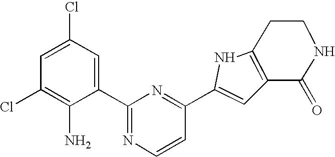 | 2-[2-(2-amino-3,5-dichlorophenyl)pyrimidin-4-yl]-1,5,6,7- tetrahydro-4H-pyrrolo[3,2-c]pyridin-4-one |
| 1060 | 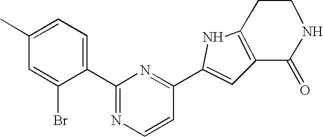 | 2-[2-(2-bromo-4-methylphenyl)pyrimidin-4-yl]-1,5,6,7- tetrahydro-4H-pyrrolo[3,2-c]pyridin-4-one |
| 1061 | 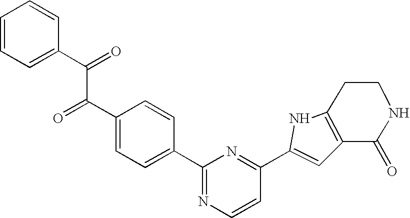 | 1-{4-[4-(4-oxo-4,5,6,7-tetrahydro-1H-pyrrolo[3,2-c]pyridin- 2-yl)pyrimidin-2-yl]phenyl}-2-phenylethane-1,2-dione |
| 1062 | 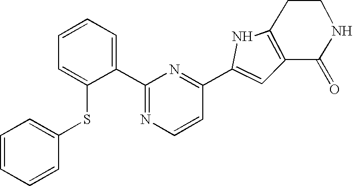 | 2-{2-[2-(phenylthio)phenyl]pyrimidin-4-yl)-1,5,6,7- tetrahydro-4H-pyrrolo[3,2-c]pyridin-4-one |
| 1063 | 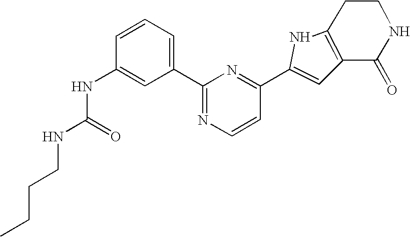 | N-butyl-N-{4-methyl-3-[4-(4-oxo-4,5,6,7-tetrahydro-1H- pyrrolo[3,2-c]pyridin-2-yl)pyrimidin-2-yl]phenyl}urea |
| 1064 | 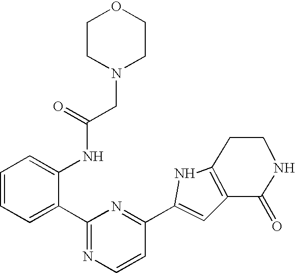 | 2-morpholin-4-yl-N-{2-[4-(4-oxo-4,5,6,7-tetrahydro-1H- pyrrolo[3,2-c]pyridin-2-yl)pyrimidin-2-yl]phenyl}acetamide |
| 1065 | 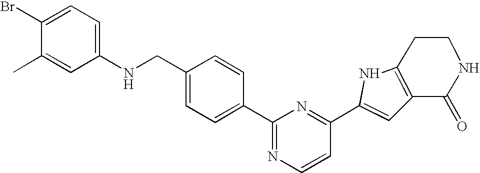 | 2-[2-(4-{[(4-bromo-3- methylphenyl)amino]methyl}phenyl)pyrimidin-4-yl}-1,5,6,7- tetrahydro-4H-pyrrolo[3,2-c]pyridin-4-one |
| 1066 | 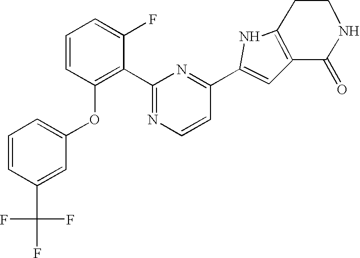 | 2-(2-{2-fluoro-6-[3- (trifluoromethyl)phenoxy]phenyl}pyrimidin-4-yl)-1,5,6,7- tetrahydro-4H-pyrrolo[3,2-c]pyridin-4-one |
| 1067 | 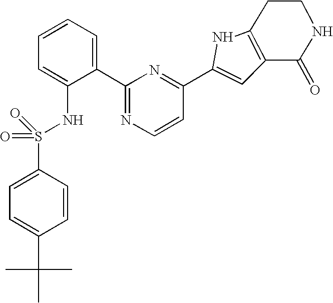 | 4-tert-butyl-N-{2-[4-(4-oxo-4,5,6,7-tetrahydro-1H- pyrrolo[3,2-c]pyridin-2-yl)pyrimidin-2- yl]phenyl}benzenesulfonamide |
| 1068 | 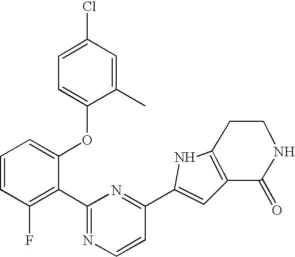 | 2-{2-[2-(4-chloro-2-methylphenoxy)-6- fluorophenyl]pyrimidin-4-yl}-1,5,6,7-tetrahydro-4H- pyrrolo[3,2-c]pyridin-4-one |
| 1069 | 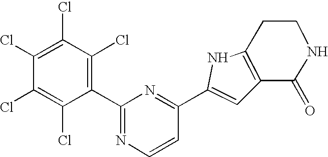 | 2-[2-(pentachlorophenyl)pyrimidin-4-yl]-1,5,6,7-tetrahydro- 4H-pyrrolo[3,2-c]pyridin-4-one |
| 1070 | 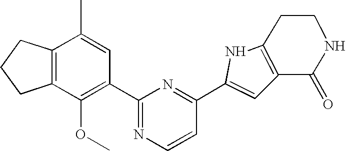 | 2-[2-(4-methoxy-7-methyl-2,3-dihydro-1H-inden-5- yl)pyrimidin-4-yl]-1,5,6,7-tetrahydro-4H-pyrrolo[3,2- c]pyridin-4-one |
| 1071 | 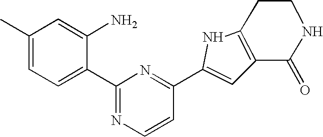 | 2-[2-(2-amino-4-methylphenyl)pyrimidin-4-yl]-1,5,6,7- tetrahydro-4H-pyrrolo[3,2-c]pyridin-4-one |
| 1072 | 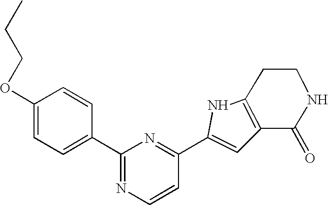 | 2-[2-(4-propoxyphenyl)pyrimidin-4-yl]-1,5,6,7-tetrahydro- 4H-pyrrolo[3,2-c]pyridin-4-one |
| 1073 | 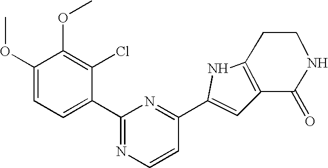 | 2-[2-(2-chloro-3,4-dimethoxyphenyl)pyrimidin-4-yl]-1,5,6,7- tetrahydro-4H-pyrrolo[3,2-c]pyridin-4-one |
| 1074 | 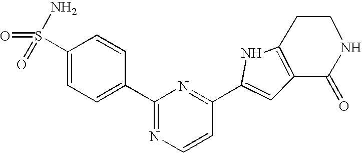 | 4-[4-(4-oxo-4,5,6,7-tetrahydro-1H-pyrrolo[3,2-c]pyridin-2- yl)pyrimidin-2-yl]benzenesuifonamide |
| 1075 | 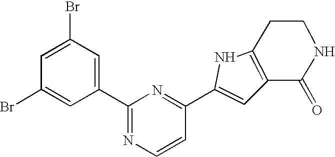 | 2-[2-(3,5-dibromophenyl)pyrimidin-4-yl]-1,5,6,7-tetrahydro- 4H-pyrrolo[3,2-c]pyridin-4-one |
| 1076 | 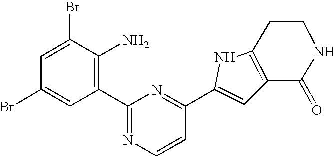 | 2-[2-(2-amino-3,5-dibromophenyl)pyrimidin-4-yl]-1,5,6,7- tetrahydro-4H-pyrrolo[3,2-c]pyridin-4-one |
| 1077 | 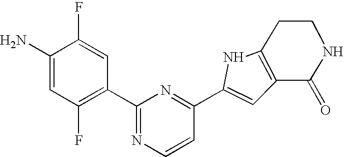 | 2-[2-(4-amino-2,5-difluorophenyl)pyrimidin-4-yl]-1,5,6,7- tetrahydro-4H-pyrrolo[3,2-c]pyridin-4-one |
| 1078 | 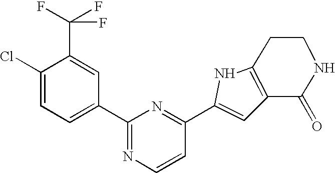 | 2-{2-[4-chloro-3-(trifluoromethyl)phenyl]pyrimidin-4-yl}- 1,5,6,7-tetrahydro-4H-pyrrolo[3,2-c]pyridin-4-one |
| 1079 | 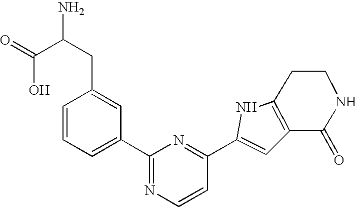 | 3-[4-(4-oxo-4,5,6,7-tetrahydro-1H-pyrrolo[3,2-c]pyridin-2- yl)pyrimidin-2-yl]phenylalanine |
| 1080 | 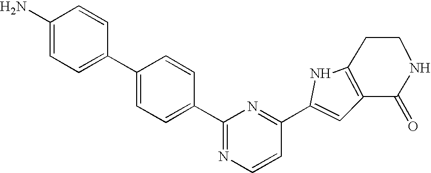 | 2-[2-(4-amino-1,1′-biphenyl-4-yl)pyrimidin-4-yl]-1,5,6,7- tetrahydro-4H-pyrrolo[3,2-c]pyridin-4-one |
| 1081 | 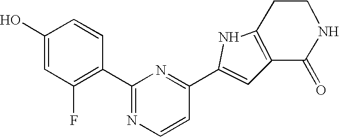 | 2-[2-(2-fluoro-4-hydroxyphenyl)pyrimidin-4-yl]-1,5,6,7- tetrahydro-4H-pyrrolo[3,2-c]pyridin-4-one |
| 1082 | 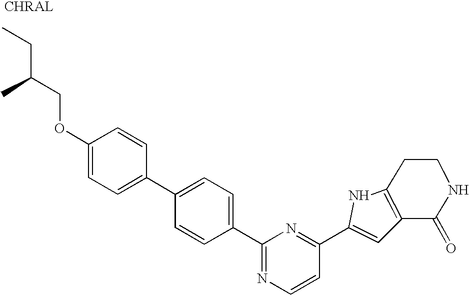 | 2-[2-(4′-{[(2S)-2-methylbutyl]oxy}-1,1′-biphenyl-4- yl)pyrimidin-4-yl]-1,5,6,7-tetrahydro-4H-pyrrolo[3,2- c]pyridin-4-one |
| 1083 | 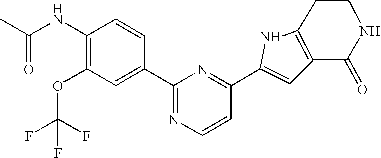 | N-[4-[4-(4-oxo-4,5,6,7-tetrahydro-1H-pyrrolo[3,2-c]pyridin- 2-yl)pyrimidin-2-yl]-2-(trifluoromethoxy)phenyl]acetamide |
| 1084 | 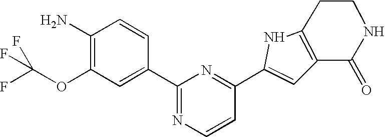 | 2-{2-[4-amino-3-(trifluoromethoxy)phenyl]pyrimidin-4-yl}- 1,5,6,7-tetrahydro-4H-pyrrolof3,2-c]pyridin-4-one |
| 1085 | 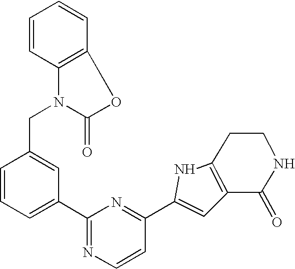 | 3-{3-[4-(4-oxo-4,5,6,7-tetrahydro-1H-pyrrolo[3,2-c]pyridin- 2-yl)pyrimidin-2-yl]benzyl]-1,3-benzoxazol-2(3H)-one |
| 1086 | 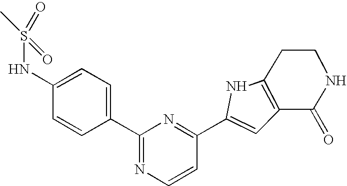 | N-{4-[4-(4-oxo-4,5,6,7-tetrahydro-1H-pyrrolo[3,2-c]pyridin- 2-yl)pyrimidin-2-yl]phenyl}methanesulfonamide |
| 1087 | 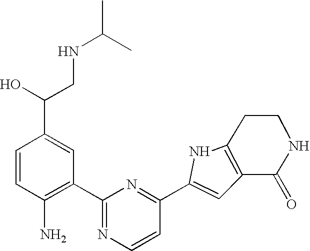 | 2-(2-{2-amino-5-[1-hydroxy-2- (isopropylamino)ethyl]phenyl}pyrimidin-4-yl)-1,5,6,7- tetrahydro-4H-pyrrolo[3,2-c]pyridin-4-one |
| 1088 | 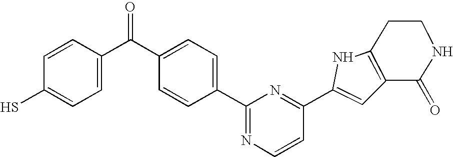 | 2-{2-[4-(4-mercaptobenzoyl)phenyl]pyrimidin-4-yl}-1,5,6,7- tetrahydro-4H-pyrrolo[3,2-c]pyridin-4-one |
| 1089 | 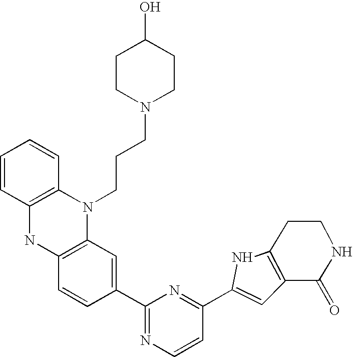 | 2-(2-{10-[3-(4-hydroxypiperidin-1-yl)propyl]-10H- phenothiazin-2-yl}pyrimidin-4-yl)-1,5,6,7-tetrahydro-4H- pyrrolo[3,2-c]pyridin-4-one |
| 1090 | 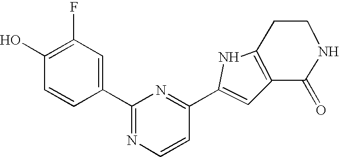 | 2-[2-(3-fluoro-4-hydroxyphenyl)pyrimidin-4-yl]-1,5,6,7- tetrahydro-4H-pyrrolo[3,2-c]pyridin-4-one |
| 1091 | 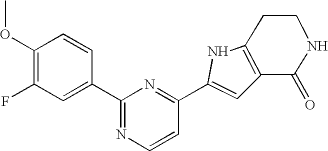 | 2-[2-(3-fluoro-4-methoxyphenyl)pyrimidin-4-yl]-1,5,6,7- tetrahydro-4H-pyrrolo[3,2-c]pyridin-4-one |
| 1092 | 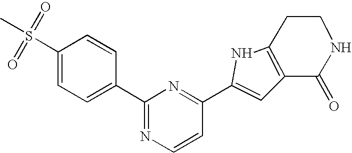 | 2-{2-(4-(methylsulfonyl)phenyl]pyrimidin-4-yl}-1,5,6,7- tetrahydro-4H-pyrrolo[3,2-c]pyridin-4-one |
| 1093 | 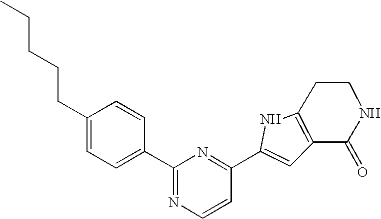 | 2-[2-(4-pentylphenyl)pyrimidin-4-yl]-1,5,6,7-tetrahydro-4H- pyrrolo[3,2-c]pyridin-4-one |
| 1094 | 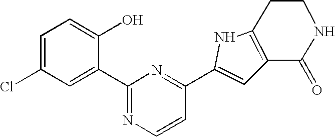 | 2-[2-(5-chloro-2-hydroxyphenyl)pyrimidin-4-yl]-1,5,6,7- tetrahydro-4H-pyrrolof3,2-c]pyridin-4-one |
| 1095 | 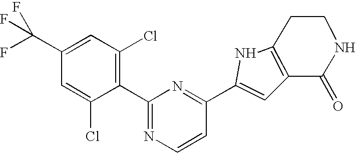 | 2-{2-[2,6-dichloro-4-(trifluoromethyl)phenyl]pyrimidin-4-yl}- 1,5,6,7-tetrahydro-4H-pyrrolo[3,2-c]pyridin-4-one |
| 1096 | 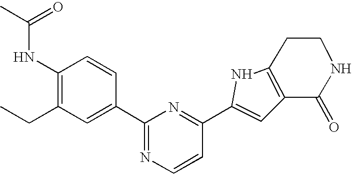 | N-{2-ethyl-4-[4-(4-oxo-4,5,6,7-tetrahydro-1H-pyrrolo[3,2- c]pyridin-2-yl)pyrimidin-2-yl]phenyl}acetamide |
| 1097 | 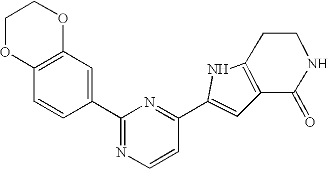 | 2-[2-(2,3-dihydro-1,4-benzodioxin-6-yl)pyrimidin-4-yl]- 1,5,6,7-tetrahydro-4H-pyrrolo[3,2-c]pyridin-4-one |
| 1098 | 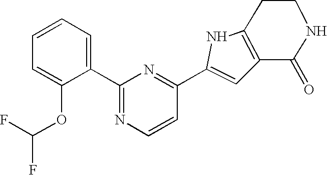 | 2-{2-[2-(difluoromethoxy)phenyl]pyrimidin-4-yl}-1,5,6,7- tetrahydro-4H-pyrrolo[3,2-c]pyridin-4-one |
| 1099 | 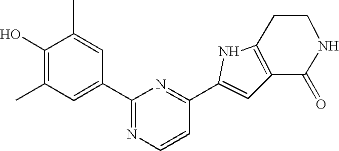 | 2-[2-(4-hydroxy-3,5-dimethylphenyl)pyrimidin-4-yl]-1,5,6,7- tetrahydro-4H-pyrrolo[3,2-c]pyridin-4-one |
| 1100 | 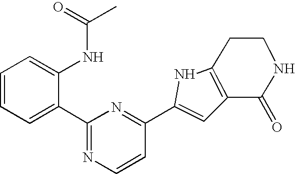 | N-{2-[4-(4-oxo-4,5,6,7-tetrahydro-1H-pyrrolo[3,2-c]pyridin- 2-yl)pyrimidin-2-yl]phenyl}acetamide |
| 1101 | 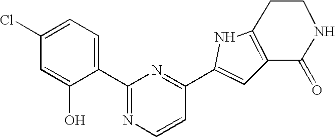 | 2-[2-(4-chloro-2-hydroxyphenyl)pyrimidin-4-yl]-1,5,6,7- tetrahydro-4H-pyrrolo[3,2-c]pyridin-4-one |
| 1102 | 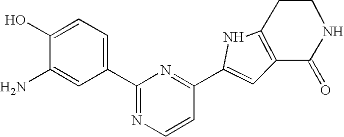 | 2-[2-(3-amino-4-hydroxyphenyl)pyrimidin-4-yl]-1,5,6,7- tetrahydro-4H-pyrrolo[3,2-c]pyridin-4-one |
| 1103 | 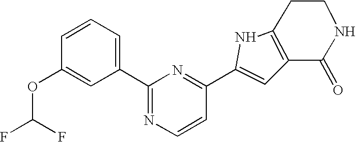 | 2-{2-[3-(difiuoromethoxy)phenyl]pyrimidin-4-yl}-1,5,6,7- tetrahydro-4H-pyrrolo[3,2-c]pyridin-4-one |
| 1104 | 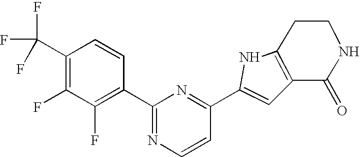 | 2-{2-[2,3-difluoro-4-(trifluoromethyl)phenyl]pyrimidin-4-yl)- 1,5,6,7-tetrahydro-4H-pyrrolo[3,2-c]pyridin-4-one |
| 1105 | 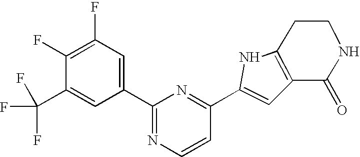 | 2-{2-[3,4-difluoro-5-(trifluoromethyl)phenyl]pyrimidin-4-yl}- 1,5,6,7-tetrahydro-4H-pyrrolo[3,2-c]pyridin-4-one |
| 1106 | 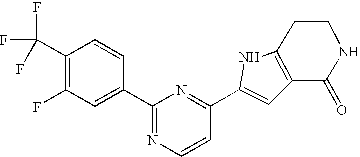 | 2-{2-[3-fluoro-4-(trifluoromethyl)phenyl]pyrimidin-4-yl}- 1,5,6,7-tetrahydro-4H-pyrrolo[3,2-c]pyridin-4-one |
| 1107 | 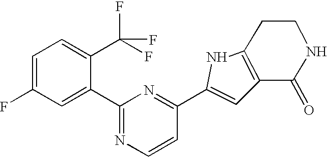 | 2-{2-[5-fluoro-2-(trifluoromethyl)phenyl]pyrimidin-4-yl}- 1,5,6,7-tetrahydro-4H-pyrrolo[3,2-c]pyridin-4-one |
| 1108 | 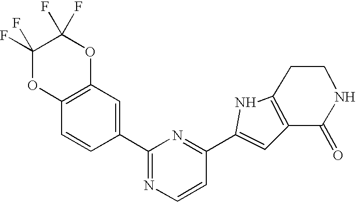 | 2-[2-(2,2,3,3-tetrafluoro-2,3-dihydro-1,4-benzodioxin-6- yl)pyrimidin-4-yl]-1,5,6,7-tetrahydro-4H-pyrrolo[3,2- c]pyridin-4-one |
| 1109 | 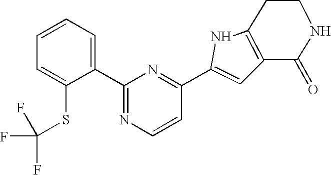 | 2-(2-{2-[(trifluoromethyl)thio]phenyl}pyrimidin-4-yl)-1,5,6,7- tetrahydro-4H-pyrrolo[3,2-c]pyridin-4-one |
| 1110 | 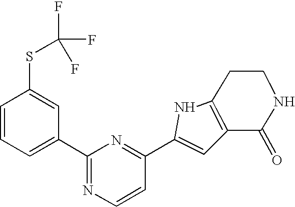 | 2-(2-{3-[(trifluoromethyl)thio]phenyl]pyrimidin-4-yl)-1,5,6,7- tetrahydro-4H-pyrrolo[3,2-c]pyridin-4-one |
| 1111 | 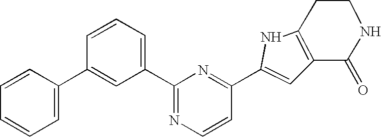 | 2-[2-(1,1′-biphenyl-3-yl)pyrimidin-4-yl]-1,5,6,7-tetrahydro- 4H-pyrrolo[3,2-c]pyridin-4-one |
| 1112 | 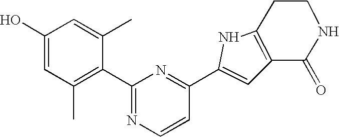 | 2-[2-(4-hydroxy-2,6-dimethylphenyl)pyrimidin-4-yl]-1,5,6,7- tetrahydro-4H-pyrrolo[3,2-c]pyridin-4-one |
| 1113 | 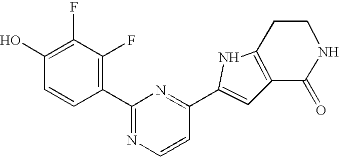 | 2-[2-(2,3-difluoro-4-hydroxyphenyl)pyrimidin-4-yl]-1,5,6,7- tetrahydro-4H-pyrrolof3,2-c]pyridin-4-one |
| 1114 | 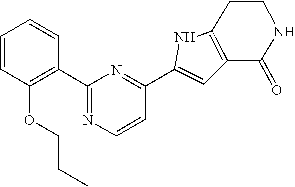 | 2-[2-(2-propoxyphenyl)pyrimidin-4-yl]-1,5,6,7-tetrahydro- 4H-pyrrolo[3,2-c]pyridin-4-one |
| 1115 | 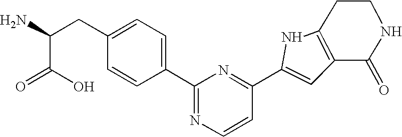 | 4-[4-(4-oxo-4,5,6,7-tetrahydro-1H-pyrrolo[3,2-c]pyridin-2- yl)pyrimidin-2-yl]-L-phenylalanine |
| 1116 | 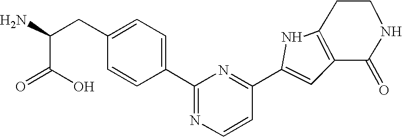 | 4-[4-(4-oxo-4,5,6,7-tetrahydro-1H-pyrrolo[3,2-c]pyridin-2- yl)pyrimidin-2-yl]-D-phenylalanine |
| 1117 | 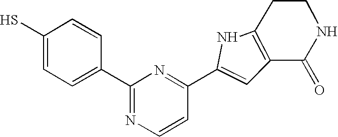 | 2-[2-(4-mercaptophenyl)pyrimidin-4-yl]-1,5,6,7-tetrahydro- 4H-pyrrolo[3,2-c]pyridin-4-one |
| 1118 | 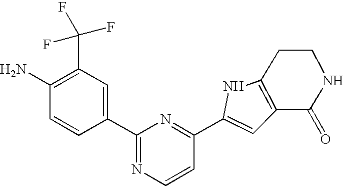 | 2-{2-[4-amino-3-(trifluoromethyl)phenyl]pyrimidin-4-yl}- 1,5,6,7-tetrahydro-4H-pyrrolo[3,2-c]pyridin-4-one |
| 1119 | 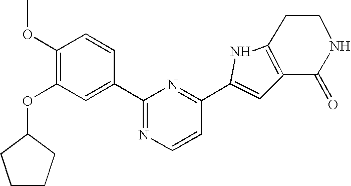 | 2-{2-[3-(cyclopentyloxy)-4-methoxyphenyl]pyrimidin-4-yl}- 1,5,6,7-tetrahydro-4H-pyrrolo[3,2-c]pyridin-4-one |
| 1120 | 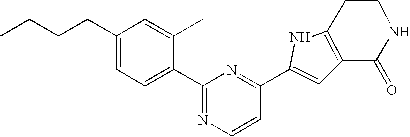 | 2-[2-(4-butyl-2-methylphenyl)pyrimidin-4-yl]-1,5,6,7- tetrahydro-4H-pyrrolo[3,2-c]pyridin-4-one |
| 1121 | 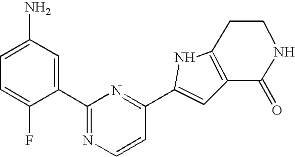 | 2-[2-(5-amino-2-fluorophenyl)pyrimidin-4-yl]-1,5,6,7- tetrahydro-4H-pyrrolo[3,2-c]pyridin-4-one |
| 1122 | 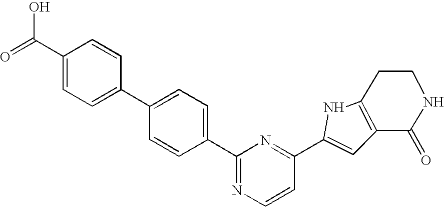 | 4-[4-(4-oxo-4,5,6,7-tetrahydro-1H-pyrrolo[3,2-c]pyridin-2- yl)pyrimidin-2-yl]-1,1′-biphenyl-4-carboxylic acid |
| 1123 | 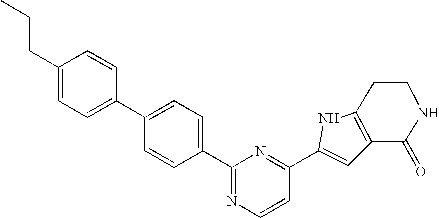 | 2-[2-(4-propy-1,1′-biphenyl-4-yl)pyrimidin-4-yl]-1,5,6,7- tetrahydro-4H-pyrrolo[3,2-c]pyridin-4-one |
| 1124 | 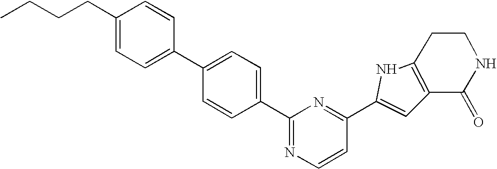 | 2-[2-(4′-butyl-1,1′-biphenyl-4-yl)pyrimidin-4-yl]-1,5,6,7- tetrahydro-4H-pyrrolo[3,2-c]pyridin-4-one |
| 1125 | 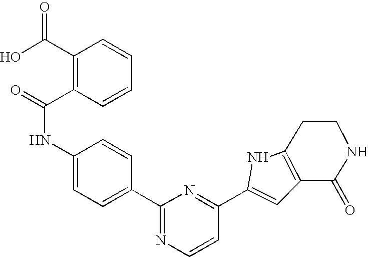 | 2-[({4-[4-(4-oxo-4,5,6,7-tetrahydro-1H-pyrrolo[3,2- c]pyridin-2-yl)pyrimidin-2- yl]phenyl}amino)carbonyl]benzoic acid |
| 1126 | 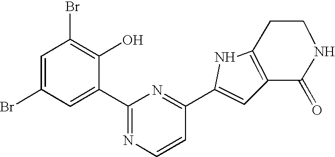 | 2-[2-(3,5-dibromo-2-hydroxyphenyl)pyrimidin-4-yl]-1,5,6,7- tetrahydro-4H-pyrrolo[3,2-c]pyridin-4-one |
| 1127 | 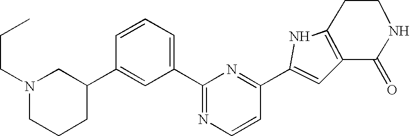 | 2-(2-{3-[(3S)-1-propylpiperidin-3-yl]phenyl}pyrimidin-4-yl)- 1,5,6,7-tetrahydro-4H-pyrrolo[3,2-c]pyridin-4-one |
| 1128 | 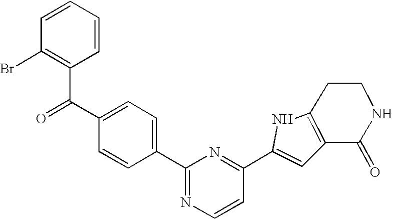 | 2-{2-[4-(2-bromobenzoyl)phenyl]pyrimidin-4-yl}-1,5,6,7- tetrahydro-4H-pyrrolo[3,2-c]pyridin-4-one |
| 1129 | 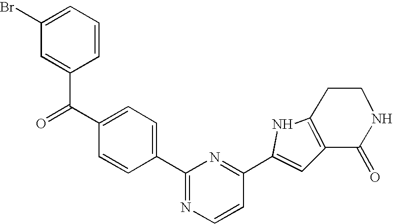 | 2-{2-[4-(3-bromobenzoyl)phenyl]pyrimidin-4-yl}-1,5,6,7- tetrahydro-4H-pyrrolo[3,2-c]pyridin-4-one |
| 1130 | 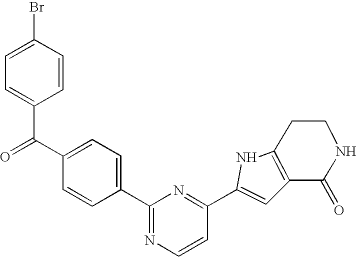 | 2-{2-[4-(4-bromobenzoyl)phenyl]pyrimidin-4-yl}-1,5,6,7- tetrahydro-4H-pyrrolo[3,2-c]pyridin-4-one |
| 1131 | 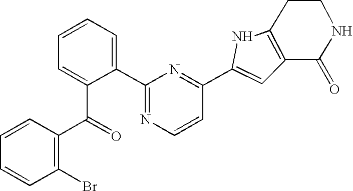 | 2-{2-[2-(2-bromobenzoyl)phenyl]pyrimidin-4-yl}-1,5,6,7- tetrahydro-4H-pyrrolo[3,2-c]pyridin-4-one |
| 1132 | 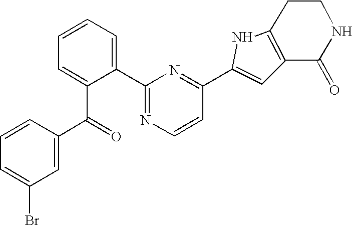 | 2-{2-[2-(3-bromobenzoyl)phenyl]pyrimidin-4-yl}-1,5,6,7- tetrahydro-4H-pyrrolo[3,2-c]pyridin-4-one |
| 1133 | 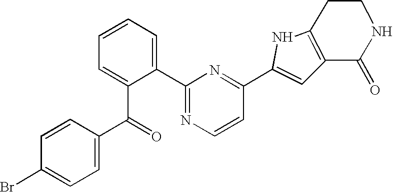 | 2-{2-[2-(4-bromobenzoyl)phenyl]pyrimidin-4-yl}-1,5,6,7- tetrahydro-4H-pyrrolo[3,2-c]pyridin-4-one |
| 1134 | 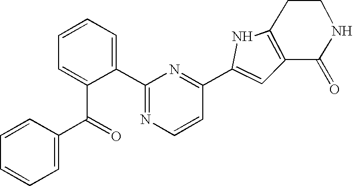 | 2-[2-(2-benzoylphenyl)pyrimidin-4-yl]-1,5,6,7-tetrahydro- 4H-pyrrolo[3,2-c]pyridin-4-one |
| 1135 | 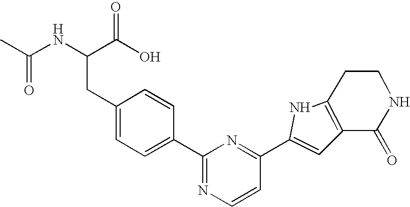 | N-acetyl-4-[4-(4-oxo-4,5,6,7-tetrahydro-1H-pyrrolo[3,2- c]pyridin-2-yl)pyrimidin-2-yl]-L-phenylalanine |
| 1136 | 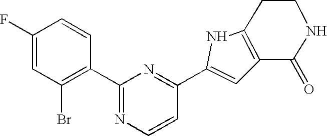 | 2-[2-(2-bromo-4-fluorophenyl)pyrimidin-4-yl]-1,5,6,7- tetrahydro-4H-pyrrolo[3,2-c]pyridin-4-one |
| 1137 | 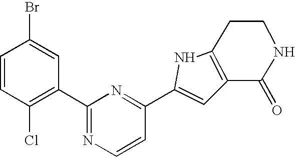 | 2-[2-(5-bromo-2-chlorophenyl)pyrimidin-4-yl]-1,5,6,7- tetrahydro-4H-pyrrolor3,2-c]pyridin-4-one |
| 1138 | 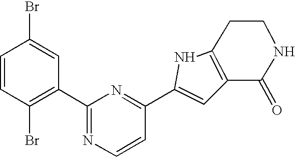 | 2-[2-(2,5-dibromophenyl)pyrimidin-4-yl]-1,5,6,7-tetrahydro- 4H-pyrrolo[3,2-c]pyridin-4-one |
| 1139 | 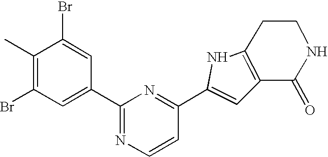 | 2-[2-(3,5-dibromo-4-methylphenyl)pyrimidin-4-yl]-1,5,6,7- tetrahydro-4H-pyrrolo[3,2-c]pyridin-4-one |
| 1140 | 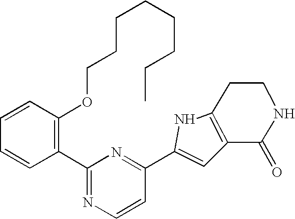 | 2-{2-[2-(octyloxy)phenyl]pyrimidin-4-yl}-1,5,6,7-tetrahydro- 4H-pyrrolo[3,2-c]pyridin-4-one |
| 1141 | 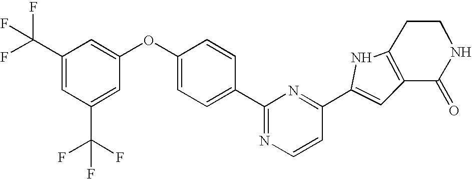 | 2-(2-{4-[3,5-bis(trifluoromethyl)phenoxy]phenyl}pyrimidin- 4-yl)-1,5,6,7-tetrahydro-4H-pyrrolo[3,2-c]pyridin-4-one |
| 1142 | 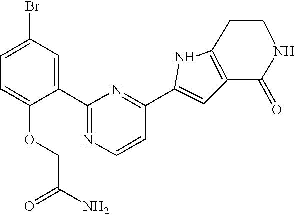 | 2-{4-bromo-2-[4-(4-oxo-4,5,6,7-tetrahydro-1H-pyrrolo[3,2- c]pyridin-2-yl)pyrimidin-2-yl]phenoxylacetamide |
| 1143 | 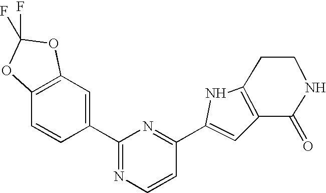 | 2-[2-(2,2-difluoro-1,3-benzodioxol-5-yl)pyrimidin-4-yl]- 1,5,6,7-tetrahydro-4H-pyrrolo[3,2-c]pyridin-4-one |
| 1144 | 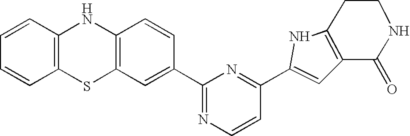 | 2[2-(10H-phenothiazin-3-yl)pyrimidin-4-yl]-1,5,6,7- tetrahydro-4H-pyrrolo[3,2-c]pyridin-4-one |
| 1145 | 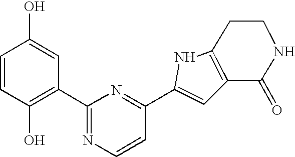 | 2-[2-(2,5-dihydroxyphenyl)pyrimidin-4-yl]-1,5,6,7- tetrahydro-4H-pyrrolo[3,2-c]pyridin-4-one |
| 1146 | 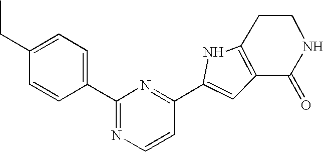 | 2-[2-(4-propylphenyl)pyrimidin-4-yl]-1,5,6,7-tetrahydro-4H- pyrrolo[3,2-c]pyridin-4-one |
| 1147 | 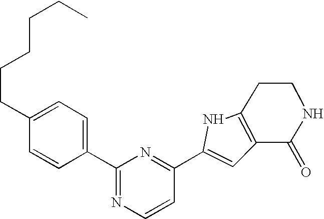 | 2-[2-(4-hexylphenyl)pyrimidin-4-yl]-1,5,6,7-tetrahydro-4H- pyrrolo[3,2-c]pyridin-4-one |
| 1148 | 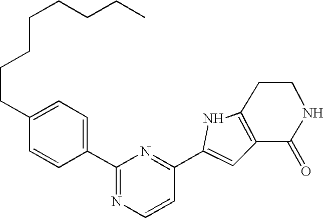 | 2-[2-(4-octylphenyl)pyrimidin-4-yl]-1,5,6,7-tetrahydro-4H- pyrrolo[3,2-c]pyridin-4-one |
| 1149 | 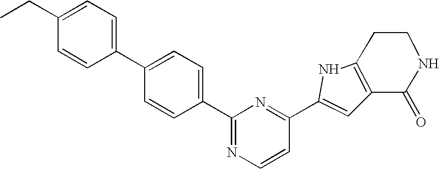 | 2-[2-(4′-ethyl-1,1′-biphenyl-4-yl)pyrimidin-4-yl]-1,5,6,7- tetrahydro-4H-pyrrolo[3,2-c]pyridin-4-one |
| 1150 | 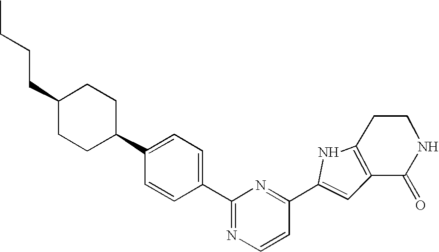 | 2-{2-[4-(4-butylcyclohexyl)phenyl]pyrimidin-4-yl}-1,5,6,7- tetrahydro-4H-pyrrolo[3,2-c]pyridin-4-one |
| 1151 | 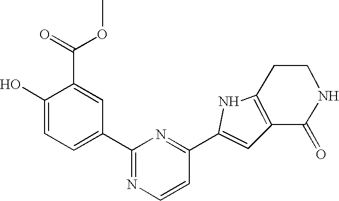 | methyl 2-hydroxy-5-[4-(4-oxo-4,5,6,7-tetrahydro-1H- pyrrolo[3,2-c]pyridin-2-yl)pyrimidin-2-yl]benzoate |
| 1152 | 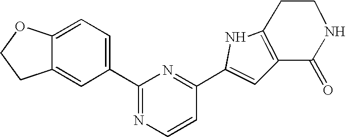 | 2-[2-(2,3-dihydro-1-benzofuran-5-yl)pyrimidin-4-yl]-1,5,6,7- tetrahydro-4H-pyrrolo[3,2-c]pyridin-4-one |
| 1153 | 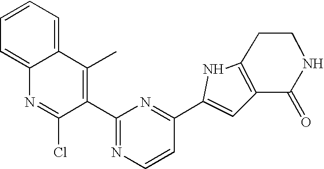 | 2-[2-(2-chloro-4-methylquinolin-3-yl)pyrimidin-4-yl]-1,5,6,7- tetrahydro-4H-pyrrolo[3,2-c]pyridin-4-one |
| 1154 | 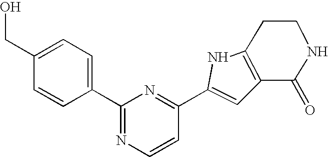 | 2-{2-[4-(hydroxymethyl)phenyl]pyrimidin-4-yl}-1,5,6,7- tetrahydro-4H-pyrrolo[3,2-c]pyridin-4-one |
| 1155 | 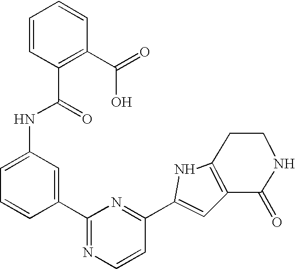 | 2-[({3-[4-(4-oxo-4,5,6,7-tetrahydro-1H-pyrrolo[3,2- c]pyridin-2-yl)pyrimidin-2- yl]phenyl}amino)carbonyl]benzoic acid |
| 1156 | 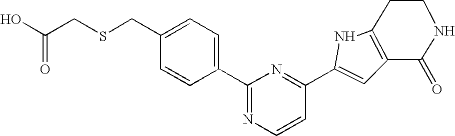 | ({4-[4-(4-oxo-4,5,6,7-tetrahydro-1H-pyrrolo[3,2-c]pyridin-2- yl)pyrimidin-2-yl]benzyl}thio)acetic acid |
| 1157 | 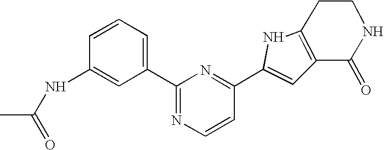 | N-{3-[4-(4-oxo-4,5,6,7-tetrahydro-1H-pyrrolo[3,2-c]pyridin- 2-yl)pyrimidin-2-yl]phenyl}acetamide |
| 1158 | 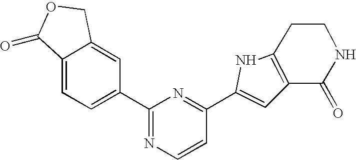 | 2-[2-(1-oxo-1,3-dihydro-2-benzofuran-5-yl)pyrimidin-4-yl]- 1,5,6,7-tetrahydro-4H-pyrrolo[3,2-c]pyridin-4-one |
| 1159 | 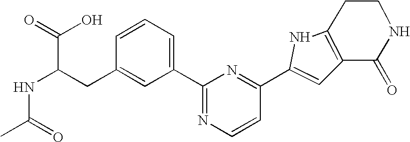 | N-acetyl-3-[4-(4-oxo-4,5,6,7-tetrahydro-1H-pyrrolo[3,2- c]pyridin-2-yl)pyrimidin-2-yllphenylalanine |
| 1160 | 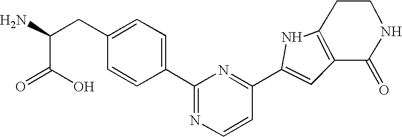 | 4-[4-(4-oxo-4,5,6,7-tetrahydro-1H-pyrrolo[3,2-c]pyridin-2- yl)pyrimidin-2-yl]-L-phenylalanine |
| 1161 | 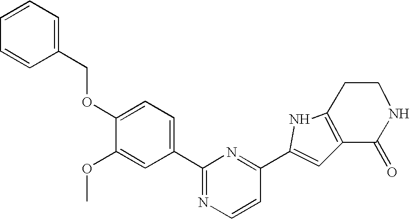 | 2-{2-[4-(benzyloxy)-3-methoxyphenyl]pyrimidin-4-yl}- 1,5,6,7-tetrahydro-4H-pyrrolo[3,2-c]pyridin-4-one |
| 1162 | 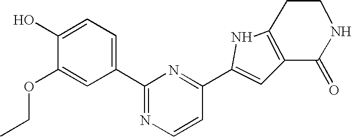 | 2-[2-(3-ethoxy-4-hydroxyphenyl)pyrimidin-4-yl]-1,5,6,7- tetrahydro-4H-pyrrolo[3,2-c]pyridin-4-one |
| 1163 | 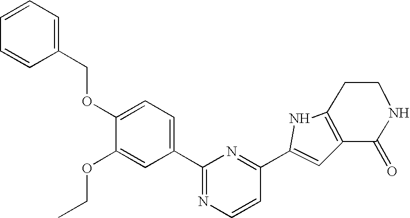 | 2-{2-[4-(benzyloxy)-3-ethoxyphenyl]pyrimidin-4-yl}-1,5,6,7- tetrahydro-4H-pyrrolo[3,2-c]pyridin-4-one |
| 1164 | 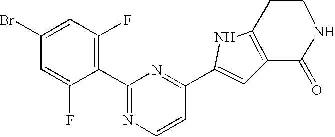 | 2-[2-(4-bromo-2,6-difluorophenyl)pyrimidin-4-yl]-1,5,6,7- tetrahydro-4H-pyrrolo[3,2-c]pyridin-4-one |
| 1165 | 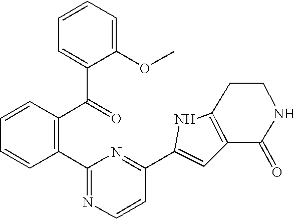 | 2-{2-[2-(2-methoxybenzoyl)phenyl]pyrimidin-4-yl}-1,5,6,7- tetrahydro-4H-pyrrolo[3,2-c]pyridin-4-one |
| 1166 | 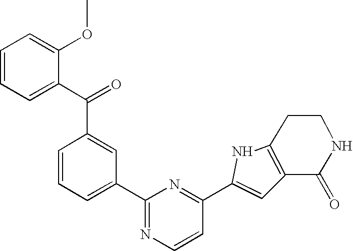 | 2-{2-[3-(2-methoxybenzoyl)phenyl]pyrimidin-4-yl}-1,5,6,7- tetrahydro-4H-pyrrolo[3,2-c]pyridin-4-one |
| 1167 | 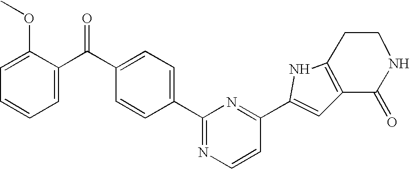 | 2-(2-[4-(2-methoxybenzoyl)phenyl]pyrimidin-4-yl}-1,5,6,7- tetrahydro-4H-pyrrolo[3,2-c]pyridin-4-one |
| 1168 | 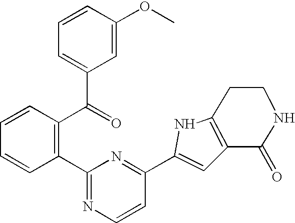 | 2-{2-[2-(3-methoxybenzoyl)phenyl]pyrimidin-4-yl}-1,5,6,7- tetrahydro-4H-pyrrolo[3,2-c]pyridin-4-one |
| 1169 | 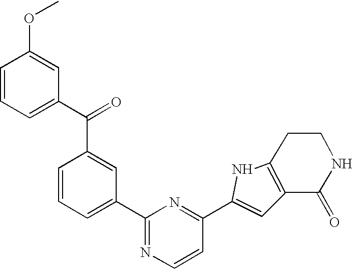 | 2-{2-[3-(3-methoxybenzoyl)phenyl]pyrimidin-4-yl)-1,5,6,7- tetrahydro-4H-pyrrolo[3,2-c]pyridin-4-one |
| 1170 | 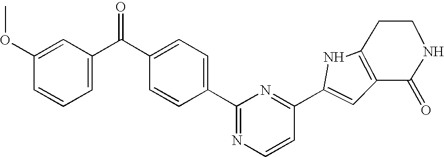 | 2-{2-[4-(3-methoxybenzoyl)phenyl]pyrimidin-4-yl}-1,5,6,7- tetrahydro-4H-pyrrolo[3,2-c]pyridin-4-one |
| 1171 | 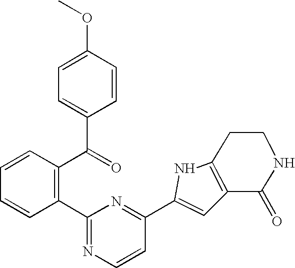 | 2-{2-[2-(4-methoxybenzoyl)phenyl]pyrimidin-4-yl}-1,5,6,7- tetrahydro-4H-pyrrolo[3,2-c]pyridin-4-one |
| 1172 | 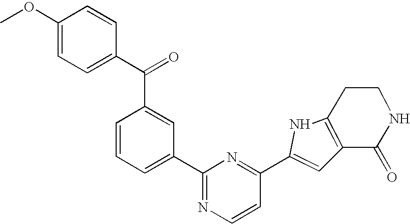 | 2-{2-[3-(4-methoxybenzoyl)phenyl]pyrimidin-4-yl}-1,5,6,7- tetrahydro-4H-pyrrolo[3,2-c]pyridin-4-one |
| 1173 | 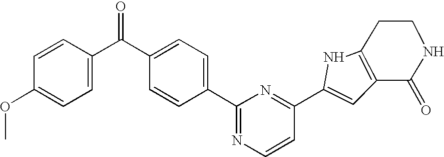 | 2-{2-[4-(4-methoxybenzoyl)phenyl]pyrimidin-4-yl)-1,5,6,7- tetrahydro-4H-pyrrolo[3,2-c]pyridin-4-one |
| 1174 | 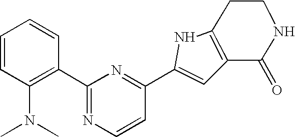 | 2-{2-[2-(dimethylamino)phenyl]pyrimidin-4-yl}-1,5,6,7- tetrahydro-4H-pyrrolo[3,2-c]pyridin-4-one |
| 1175 | 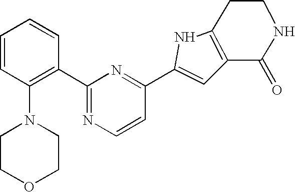 | 2-[2-(2-morpholin-4-ylphenyl)pyrimidin-4-yl]-1,5,6,7- tetrahydro-4H-pyrrolo[3,2-c]pyridin-4-one |
| 1176 | 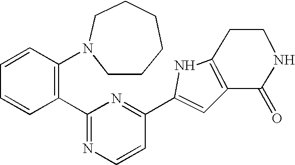 | 2-[2-(2-azepan-1-ylphenyl)pyrimidin-4-yl]-1,5,6,7- tetrahydro-4H-pyrrolo[3,2-c]pyridin-4-one |
| 1177 | 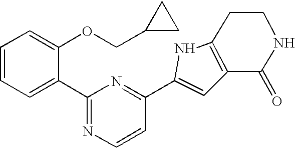 | 2-{2-[2-(cyclopropylmethoxy)phenyl]pyrimidin-4-yl}- 1,5,6,7-tetrahydro-4H-pyrrolo[3,2-c]pyridin-4-one |
| 1178 | 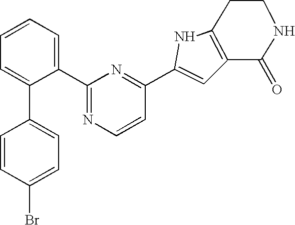 | 2-[2-(4′-bromo-1,1′-biphenyl-2-yl)pyrimidin-4-yl]-1,5,6,7- tetrahydro-4H-pyrrolo[3,2-c]pyridin-4-one |
| 1179 | 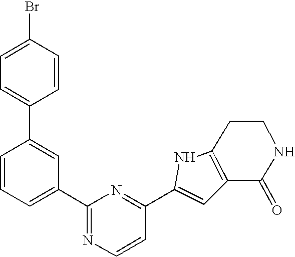 | 2-[2-(4′-bromo-1,1′-biphenyl-3-yl)pyrimidin-4-yl]-1,5,6,7- tetrahydro-4H-pyrrolo[3,2-c]pyridin-4-one |
| 1180 | 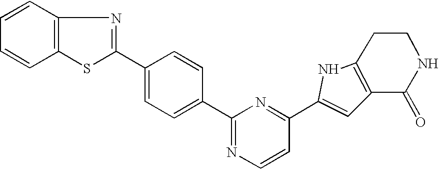 | 2-{2-[4-(1,3-benzothiazol-2-yl)phenyl]pyrimidin-4-yl}- 1,5,6,7-tetrahydro-4H-pyrrolo[3,2-c]pyridin-4-one |
| 1181 | 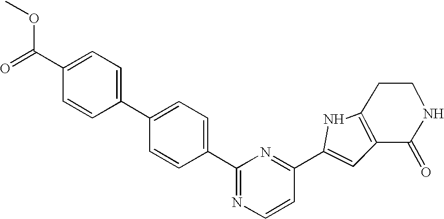 | ethyl 4′-[4-(4-oxo-4,5,6,7-tetrahydro-1H-pyrrolo[3,2- c]pyridin-2-yl)pyrimidin-2-yl]-1,1′-biphenyl-4-carboxylate |
| 1182 | 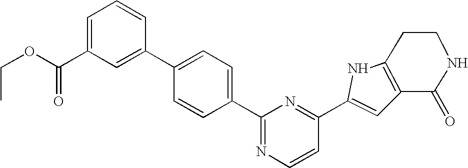 | ethyl 4′-[4-(4-oxo-4,5,6,7-tetrahydro-1H-pyrrolo[3,2- c]pyridin-2-yl)pyrimidin-2-yl]-1,1′-biphenyl-3-carboxylate |
| 1183 | 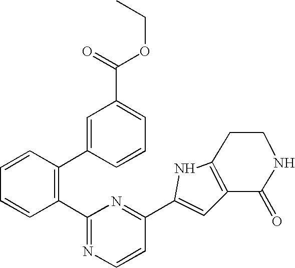 | ethyl 2′-[4-(4-oxo-4,5,6,7-tetrahydro-1H-pyrrolo[3,2- c]pyridin-2-yl)pyrimidin-2-yl]-1,1′-biphenyl-3-carboxylate c]pyridin-2-yl)pyrimidin-2-yl]-1,1′-biphenyl-3-carboxylate |
| 1184 | 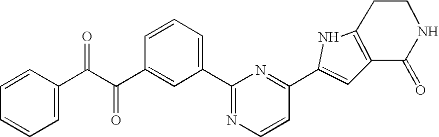 | 1-{3-[4-(4-oxo-4,5,6,7-tetrahydro-1H-pyrrolo[3,2-c]pyridin- 2-yl)pyrimidin-2-yl]phenyl}-2-phenylethane-1,2-dione |
| 1185 | 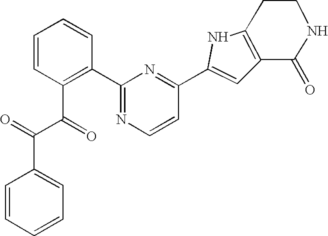 | 1-{2-[4-(4-oxo-4,5,6,7-tetrahydro-1H-pyrrolo[3,2-c]pyridin- 2-yl)pyrimidin-2-yl]phenyl]-2-phenylethane-1,2-dione |
| 1186 | 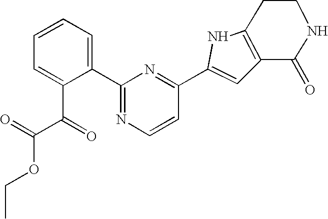 | ethyl oxo{2-[4-(4-oxo-4,5,6,7-tetrahydro-1H-pyrrolo[3,2- c]pyridin-2-yl)pyrimidin-2-yl]phenyl}acetate |
| 1187 | 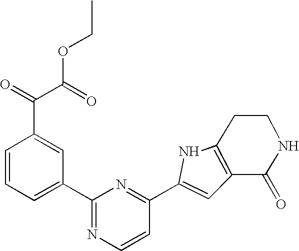 | ethyl oxo{3-[4-(4-oxo-4,5,6,7-tetrahydro-1H-pyrrolo[3,2- c]pyridin-2-yl)pyrimidin-2-yl]phenyl}acetate |
| 1188 | 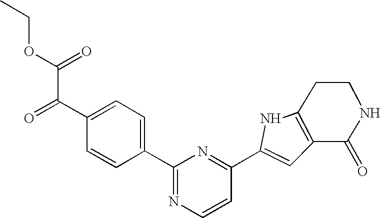 | ethyl oxo{4-[4-(4-oxo-4,5,6,7-tetrahydro-1H-pyrrolo[3,2- c]pyridin-2-yl)pyrimidin-2-yl]phenyl]acetate |
| 1189 | 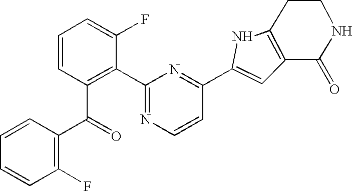 | 2-{2-[2-fluoro-6-(2-fluorobenzoyl)phenyl]pyrimidin-4-yl}- 1,5,6,7-tetrahydro-4H-pyrrolo[3,2-c]pyridin-4-one |
| 1190 | 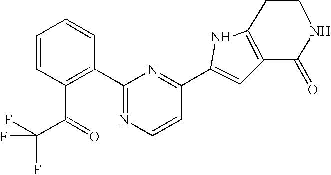 | 2-{2-[3-(trifluoroacetyl)phenyl]pyrimidin-4-yl]-1,5,6,7- tetrahydro-4H-pyrrolo[3,2-c]pyridin-4-one |
| 1191 | 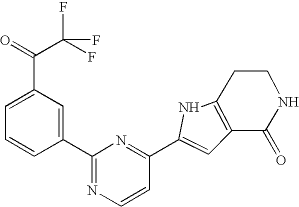 | 2-{2-[4-(trifluoroacetyl)phenyl]pyrimidin-4-yl}-1,5,6,7- tetrahydro-4H-pyrrolo[3,2-c]pyridin-4-one |
| 1192 | 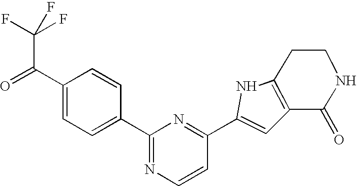 | 2-{2-[4-(trifluoroacetyl)phenyl]pyrimidin-4-yl}-1,5,6,7- tetrahydro-4H-pyrrolo[3,2-c]pyridin-4-one |
| 1193 | 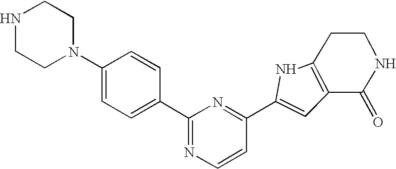 | 2-[2-(4-piperazin-1-ylphenyl)pyrimidin-4-yl]-1,5,6,7- tetrahydro-4H-pyrrolo[3,2-c]pyridin-4-one |
| 1194 | 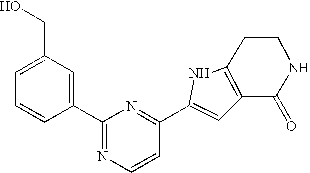 | 2-{2-[3-(hydroxymethyl)phenyl]pyrimidin-4-yl}-1,5,6,7- tetrahydro-4H-pyrrolo[3,2-c]pyridin-4-one |
| 1195 | 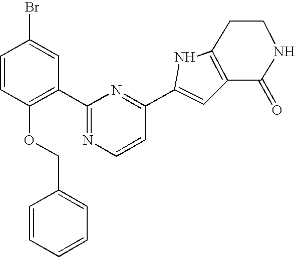 | 2-{2-[2-(benzyloxy)-5-bromophenyl]pyrimidin-4-yl}-1,5,6,7- tetrahydro-4H-pyrrolo[3,2-c]pyridin-4-one |
| 1196 | 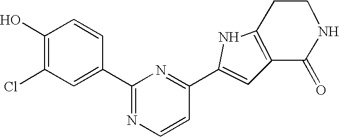 | 2-[2-(3-chloro-4-hydroxyphenyl)pyrimidin-4-yl]-1,5,6,7- tetrahydro-4H-pyrrolo[3,2-c]pyridin-4-one |
| 1197 | 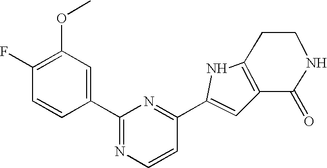 | 2-[2-(4-fluoro-3-methoxyphenyl)pyrimidin-4-yl]-1,5,6,7- tetrahydro-4H-pyrrolo[3,2-c]pyridin-4-one |
| 1198 | 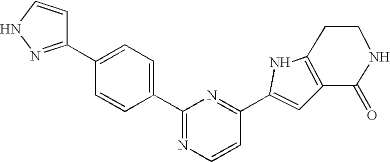 | 2-{2-[4-(1H-pyrazol-3-yl)phenyl]pyrimidin-4-yl}-1,5,6,7- tetrahydro-4H-pyrrolo[3,2-c]pyridin-4-one |
| 1199 | 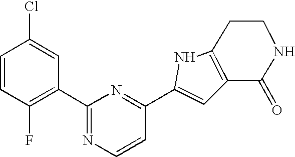 | 2-[2-(5-chloro-2-fluorophenyl)pyrimidin-4-yl]-1,5,6,7- tetrahydro-4H-pyrrolo[3,2-c]pyridin-4-one |
| 1200 | 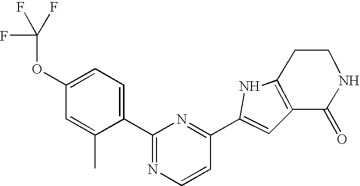 | 2-{2-[2-methyl-4-(trifluoromethoxy)phenyl]pyrimidin-4-yl}- 1,5,6,7-tetrahydro-4H-pyrrolo[3,2-c]pyridin-4-one |
| 1201 | 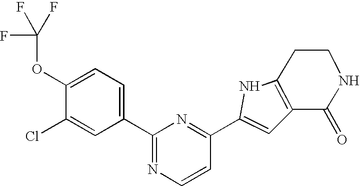 | 2-{2-[3-chloro-4-(trifluoromethoxy)phenyl]pyrimidin-4-yl}- 1,5,6,7-tetrahydro-4H-pyrrolo[3,2-c]pyridin-4-one |
| 1202 | 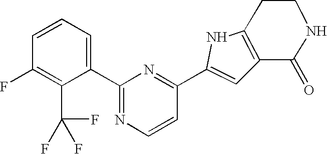 | 2-{2-[3-fluoro-2-(trifluoromethyl)phenyl]pyrimidin-4-yl}- 1,5,6,7-tetrahydro-4H-pyrrolo[3,2-c]pyridin-4-one |
| 1203 | 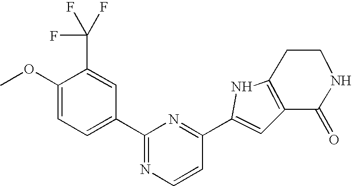 | 2-{2-[4-methoxy-3-(trifluoromethyl)phenyl]pyrimidin-4-yl]- 1,5,6,7-tetrahydro-4H-pyrrolo[3,2-c]pyridin-4-one |
| 1204 | 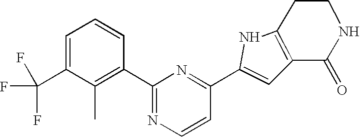 | 2-{2-[2-methyl-3-(trifluoromethyl)phenyl]pyrimidin-4-yl}- 1,5,6,7-tetrahydro-4H-pyrrolo[3,2-c]pyridin-4-one |
| 1205 | 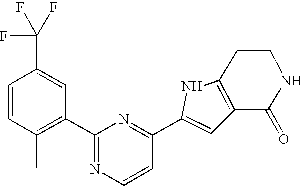 | 2-{2-[2-methyl-5-(trifluoromethyl)phenyl]pyrimidin-4-yl}- 1,5,6,7-tetrahydro-4H-pyrrolo[3,2-c]pyridin-4-one |
| 1206 | 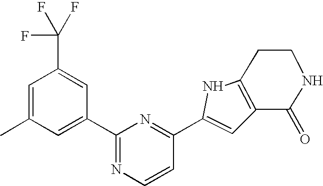 | 2-{2-[3-methyl-5-(trifiuoromethyl)phenyl]pyrimidin-4-yl}- 1,5,6,7-tetrahydro-4H-pyrrolo[3,2-c]pyridin-4-one |
| 1207 | 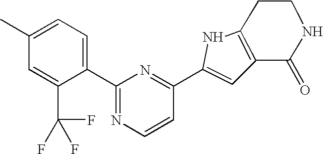 | 2-{2-[4-methyl-2-(trifluoromethyl)phenyl]pyrimidin-4-yl}- 1,5,6,7-tetrahydro-4H-pyrrolo[3,2-c]pyridin-4-one |
| 1208 | 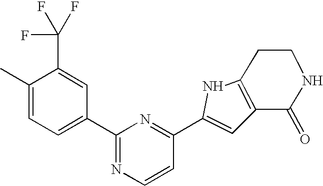 | 2-{2-[4-methyl-3-(trifluoromethyl)phenyl]pyrimidin-4-yl}- 1,5,6,7-tetrahydro-4H-pyrrolo[3,2-c]pyridin-4-one |
| 1209 | 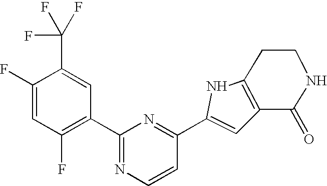 | 2-{2-[2,4-difiuoro-5-(trifluoromethyl)phenyl]pyrimidin-4-yl}- 1,5,6,7-tetrahydro-4H-pyrrolo[3,2-c]pyridin-4-one |
| 1210 | 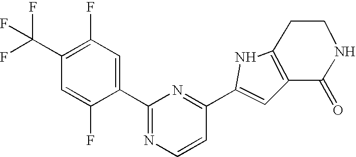 | 2-{2-[2,5-difluoro-4-(trifluoromethyl)phenyl]pyrimidin-4-yl}- 1,5,6,7-tetrahydro-4H-pyrrolo[3,2-c]pyridin-4-one |
| 1211 | 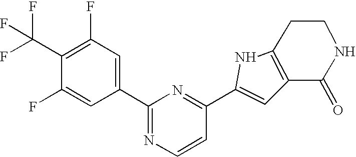 | 2-{2-[3,5-difluoro-4-(trifluoromethyl)phenyqpyrimidin-4-yl}- 1,5,6,7-tetrahydro-4H-pyrrolo[3,2-c]pyridin-4-one |
| 1212 | 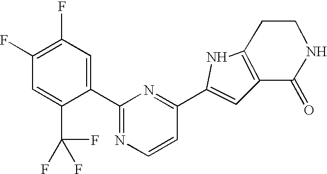 | 2-{2-[4,5-difluoro-2-(trifluoromethyl)phenyl]pyrimidin-4-yl]- 1,5,6,7-tetrahydro-4H-pyrrolo[3,2-c]pyridin-4-one |
| 1213 | 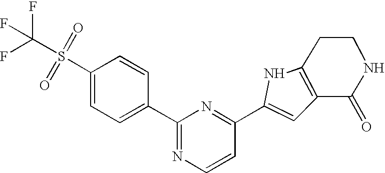 | 2-(2-{4-[(trifluoromethyl)sulfonyl]phenyl}pyrimidin-4-yl)- 1,5,6,7-tetrahydro-4H-pyrrolo[3,2-c]pyridin-4-one |
| 1214 | 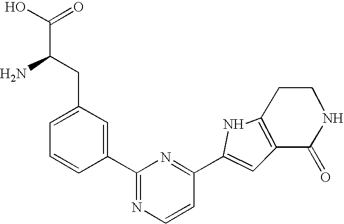 | 3-[4-(4-oxo-4,5,6,7-tetrahydro-1H-pyrrolo[3,2-c]pyridin-2- yl)pyrimidin-2-yl]-D-phenylalanine |
| 1215 | 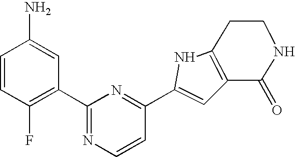 | 2-[2-(5-amino-2-fluorophenyl)pyrimidin-4-yl]-1,5,6,7- tetrahydro-4H-pyrrolo[3,2-c]pyridin-4-one |
| 1216 | 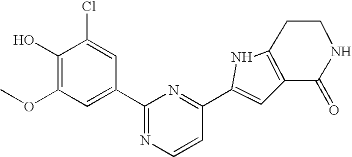 | 2-[2-(3-chloro-4-hydroxy-5-methoxyphenyl)pyrimidin-4-yl]- 1,5,6,7-tetrahydro-4H-pyrrolo[3,2-c]pyridin-4-one |
| 1217 | 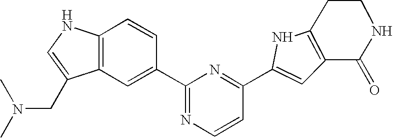 | 2-(2-{3-[(dimethylamino)methyl]-1H-indol-5-yl}pyrimidin-4- yl)-1,5,6,7-tetrahydro-4H-pyrrolo[3,2-c]pyridin-4-one |
| 1218 | 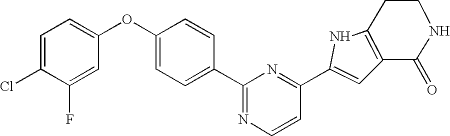 | 2-{2-[4-(4-chloro-3-fluorophenoxy)phenyl]pyrimidin-4-yl}- 1,5,6,7-tetrahydro-4H-pyrrolo[3,2-c]pyridin-4-one |
| 1219 | 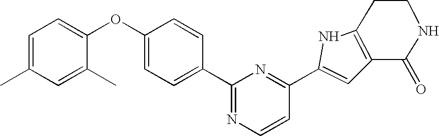 | 2-{2-[4-(2,4-dimethylphenoxy)phenyl]pyrimidin-4-yl}- 1,5,6,7-tetrahydro-4H-pyrrolo[3,2-c]pyridin-4-one |
| 1220 | 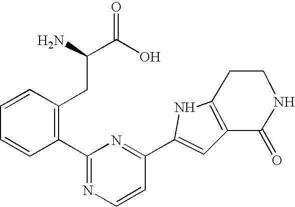 | 2-[4-(4-oxo-4,5,6,7-tetrahydro-1H-pyrrolo[3,2-c]pyridin-2- yl)pyrimidin-2-yl]-D-phenylalanine |
| 1221 | 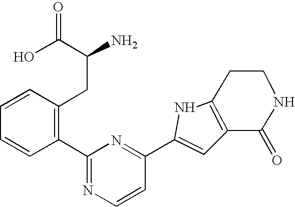 | 2-[4-(4-oxo-4,5,6,7-tetrahydro-1H-pyrrolo[3,2-c]pyridin-2- yl)pyrimidin-2-yl]-L-phenylalanine |
| 1222 | 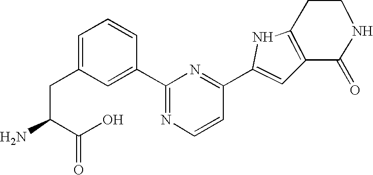 | 3-[4-(4-oxo-4,5,6,7-tetrahydro-1H-pyrrolo[3,2-c]pyridin-2- yl)pyrimidin-2-yl]-L-phenylalanine |
| 1223 | 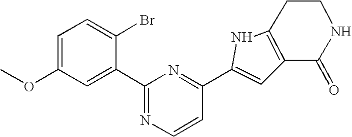 | 2-[2-(2-bromo-5-methoxyphenyl)pyrimidin-4-yl]-1,5,6,7- tetrahydro-4H-pyrrolo[3,2-c]pyridin-4-one |
| 1224 | 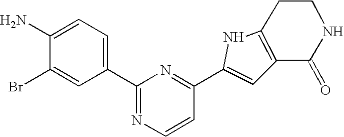 | 2-[2-(4-amino-3-bromophenyl)pyrimidin-4-yl]-1,5,6,7- tetrahydro-4H-pyrrolo[3,2-c]pyridin-4-one |
| 1225 | 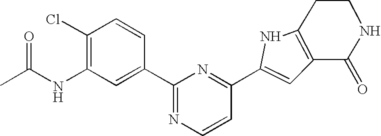 | N-{2-chloro-5-[4-(4-oxo-4,5,6,7-tetrahydro-1H-pyrrolo[3,2- c]pyridin-2-yl)pyrimidin-2-yl]phenyl}acetamide |
| 1226 | 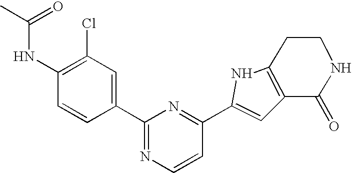 | N-{2-chloro-4-[4-(4-oxo-4,5,6,7-tetrahydro-1H-pyrrolo[3,2- c]pyridin-2-yl)pyrimidin-2-yl]phenyl}acetamide |
| 1227 | 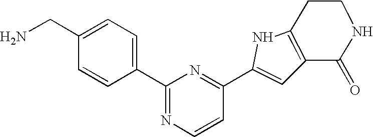 | 2-{2-[4-(aminomethyl)phenyl]pyrimidin-4-yl}-1 5,6,7- tetrahydro-4H-pyrrolo[3,2-c]pyridin-4-one |
| 1228 | 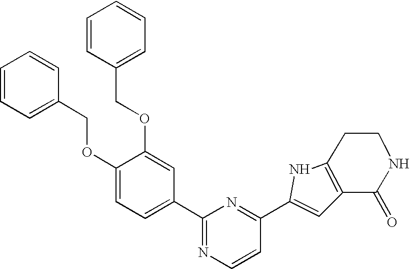 | 2-{2-[3,4-bis(benzyloxy)phenyl]pyrimidin-4-yl)-1,5,6,7- tetrahydro-4H-pyrrolo[3,2-c]pyridin-4-one |
| 1229 | 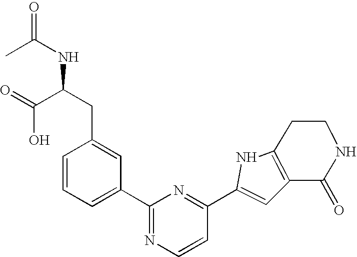 | N-acetyl-3-[4-(4-oxo-4,5,6,7-tetrahydro-1H-pyrrolo[3,2- c]pyridin-2-yl)pyrimidin-2-yl]-L-phenylalanine |
| 1230 | 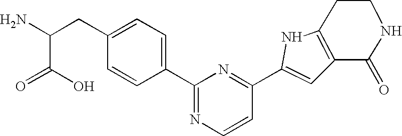 | 4-[4-(4-oxo-4,5,6,7-tetrahydro-1H-pyrrolo[3,2-c]pyridin-2- yl)pyrimidin-2-yl]phenylalanine |
| 1231 | 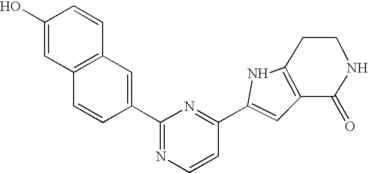 | 2-[2-(6-hydroxy-2-naphthyl)pyrimidin-4-yl]-1,5,6,7- tetrahydro-4H-pyrrolo[3,2-c]pyridin-4-one |
| 1232 | 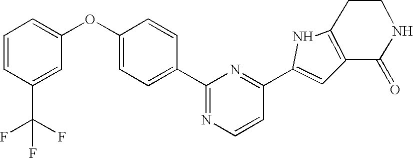 | 2-(2-{4-[3-(trifluoromethyl)phenoxy]phenyl}pyrimidin-4-yl)- 1,5,6,7-tetrahydro-4H-pyrrolo[3,2-c]pyridin-4-one |
| 1223 | 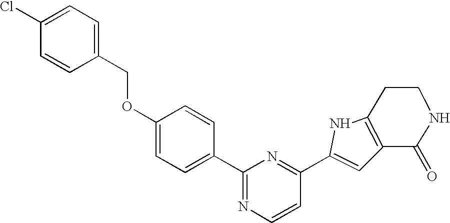 | 2-(2-{4-[(4-chlorobenzyl)oxy]phenyl}pyrimidin-4-yl)-1,5,6,7- tetrahydro-4H-pyrrolo[3,2-c]pyridin-4-one |
| 1234 | 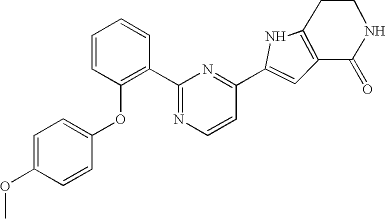 | 2-{2-[2-(4-methoxyphenoxy)phenyl]pyrimidin-4-yl}-1,5,6,7- tetrahydro-4H-pyrrolo[3,2-c]pyridin-4-one |
| 1235 | 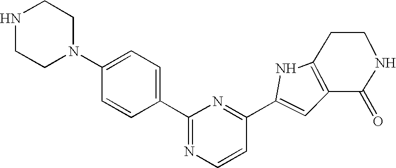 | 2-[2-(4-piperazin-1-ylphenyl)pyrimidin-4-yl]-1,5,6,7- tetrahydro-4H-pyrrolo[3,2-c]pyridin-4-one |
| 1236 | 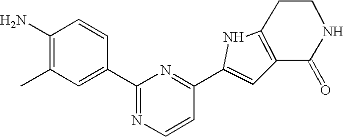 | 2-[2-(4-amino-3-methylphenyl)pyrimidin-4-yl]-1,5,6,7- tetrahydro-4H-pyrrolo[3,2-c]pyridin-4-one |
| 1237 | 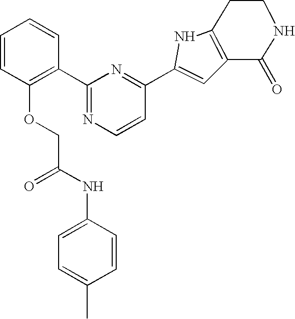 | N-(4-methylphenyl)-2-{2-[4-(4-oxo-4,5,6,7-tetrahydro-1H- pyrrolo[3,2-c]pyridin-2-yl)pyrimidin-2- yl]phenoxy}acetamide |
| 1238 | 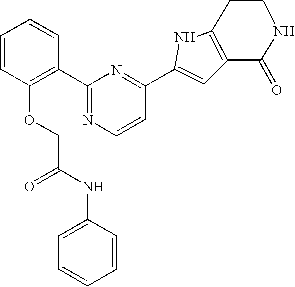 | 2-{2-[4-(4-oxo-4,5,6,7-tetrahydro-1H-pyrrolo[3,2-c]pyridin- 2-yl)pyrimidin-2-yl]phenoxy}-N-phenylacetamide |
| 1239 | 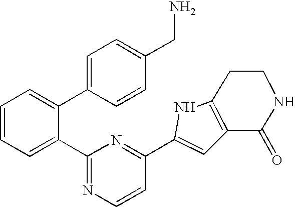 | 2-{2-[4′-(aminomethyl)-1,1′-biphenyl-2-yl]pyrimidin-4-yl}- 1,5,6,7-tetrahydro-4H-pyrrolo[3,2-c]pyridin-4-one |
| 1240 | 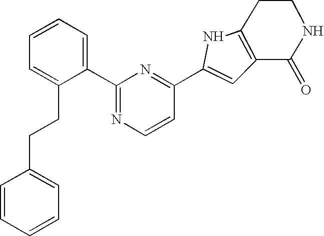 | 2-{2-[2-(2-phenylethyl)phenyl]pyrimidin-4-yl}-1,5,6,7- tetrahydro-4H-pyrrolo[3,2-c]pyridin-4-one |
| 1241 | 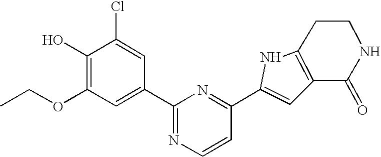 | 2-[2-(3-chloro-5-ethoxy-4-hydroxyphenyl)pyrimidin-4-yl]- 1,5,6,7-tetrahydro-4H-pyrrolo[3,2-c]pyridin-4-one |
| 1242 | 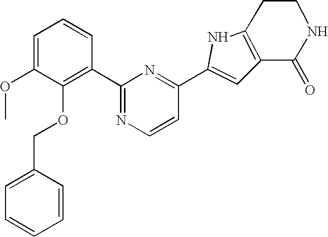 | 2-{2-[2-(benzyloxy)-3-methoxyphenyl]pyrimidin-4-yl}- 1,5,6,7-tetrahydro-4H-pyrrolo[3,2-c]pyridin-4-one |
| 1243 | 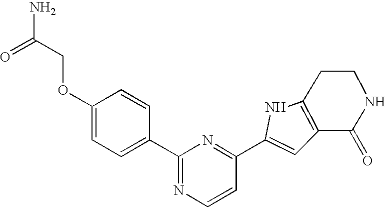 | 2-{4-[4-(4-oxo-4,5,6,7-tetrahydro-1H-pyrrolo[3,2-c]pyridin- 2-yl)pyrimidin-2-yl]phenoxy}acetamide |
| 1244 | 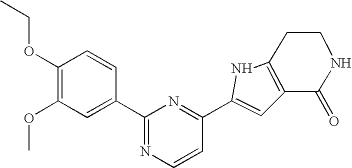 | 2-[2-(4-ethoxy-3-methoxyphenyl)pyrimidin-4-yl]-1,5,6,7- tetrahydro-4H-pyrrolo[3,2-c]pyridin-4-one |
| 1245 | 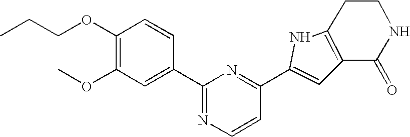 | 2-[2-(3-methoxy-4-propoxyphenyl)pyrimidin-4-yl]-1,5,6,7- tetrahydro-4H-pyrrolo[3,2-c]pyridin-4-one |
| 1246 | 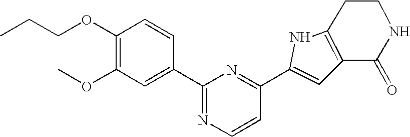 | 2-[2-(4-butoxy-3-methoxyphenyl)pyrimidin-4-yl]-1,5,6,7- tetrahydro-4H-pyrrolo[3,2-c]pyridin-4-one |
| 1247 | 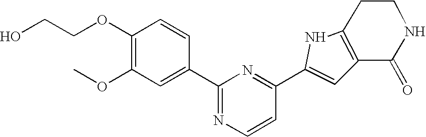 | 2-{2-[4-(2-hydroxyethoxy)-3-methoxyphenyl]pyrimidin-4- yl}-1,5,6,7-tetrahydro-4H-pyrrolo[3,2-c]pyridin-4-one |
| 1248 | 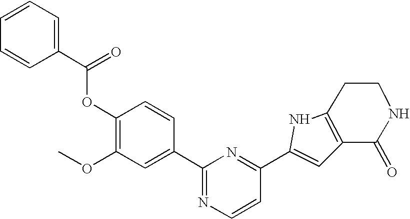 | 2-methoxy-4-[4-(4-oxo-4,5,6,7-tetrahydro-1H-pyrrolo[3,2- c]pyridin-2-yl)pyrimidin-2-yl]phenyl benzoate |
| 1249 | 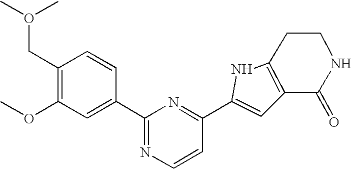 | 2-[2-(4-isopropoxy-3-methoxyphenyl)pyrimidin-4-yl]- 1,5,6,7-tetrahydro-4H-pyrrolo[3,2-c]pyridin-4-one |
| 1250 | 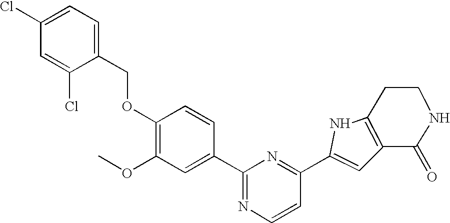 | 2-(2-{4-[(2,4-dichlorobenzyl)oxy]-3- methoxyphenyl}pyrimidin-4-yl)-1,5,6,7-tetrahydro-4H- pyrrolo[3,2-c]pyridin-4-one |
| 1251 | 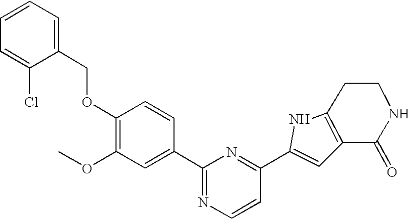 | 2-(2-{4-[(2-chlorobenzyl)oxy]-3-methoxyphenyl}pyrimidin- 4-yl)-1,5,6,7-tetrahydro-4H-pyrrolo[3,2-c]pyridin-4-one |
| 1252 | 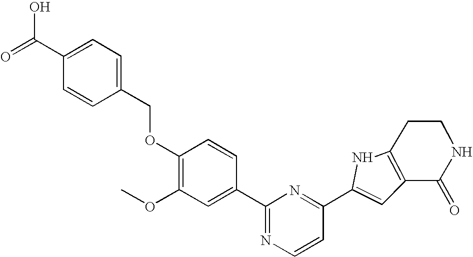 | 4-({2-methoxy-4-[4-(4-(4-oxo.4,5,6,7-tetrahydro-1H- pyrrolo[3,2-c]pyridin-2-yl)pyrimidin-2- yl]phenoxy}methyl)benzoic acid |
| 1253 | 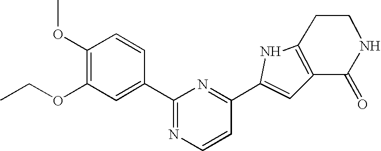 | 2-[2-(3-ethoxy-4-methoxyphenyl)pyrimidin-4-yl]-1,5,6,7- tetrahydro-4H-pyrrolo[3,2-c]pyridin-4-one |
| 1254 | 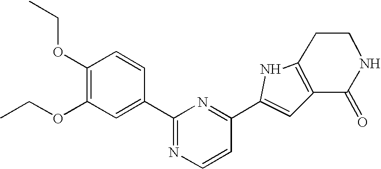 | 2-[2-(3,4-diethoxyphenyl)pyrimidin-4-yl]-1,5,6,7-tetrahydro- 4H-pyrrolo[3,2-c]pyridin-4-one |
| 1255 | 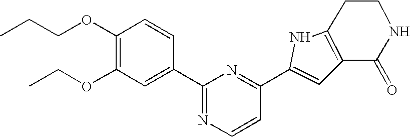 | 2-[2-(3-ethoxy-4-propoxyphenyl)pyrimidin-4-yl]-1,5,6,7- tetrahydro-4H-pyrrolo[3,2-c]pyridin-4-one |
| 1256 | 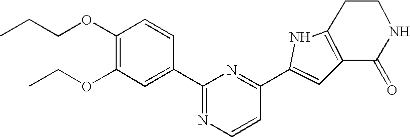 | 2-[2-(4-butoxy-3-ethoxyphenyl)pyrimidin-4-yl]-1,5,6,7- tetrahydro-4H-pyrrolo[3,2-c]pyridin-4-one |
| 1257 | 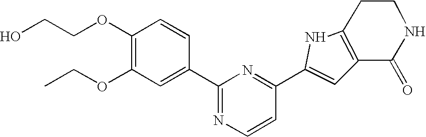 | 2-{2-[3-ethoxy-4-(2-hydroxyethoxy)phenyl]pyrimidin-4-yl}- 1,5,6,7-tetrahydro-4H-pyrrolo[3,2-c]pyridin-4-one |
| 1258 | 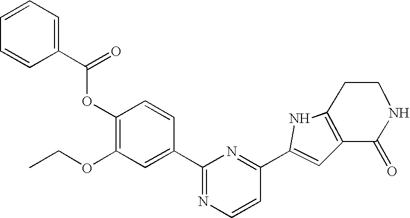 | 2-ethoxy-4-[4-(4-oxo-4,5,6,7-tetrahydro-1H-pyrrolo[3,2- c]pyridin-2-yl)pyrimidin-2-yl]phenyl benzoate |
| 1259 | 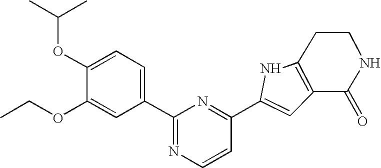 | 2-[2-(3-ethoxy-4-isopropoxyphenyl)pyrimidin-4-yl]-1,5,6,7- tetrahydro-4H-pyrrolo[3,2-c]pyridin-4-one |
| 1260 | 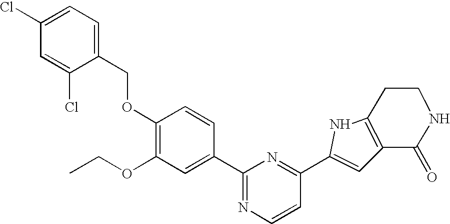 | 2-(2-{4-[(2,4-dichlorobenzyl)oxy]-3- ethoxyphenyl}pyrimidin-4-yl)-1,5,6,7-tetrahydro-4H- pyrrolo[3,2-c]pyridin-4-one |
| 1261 | 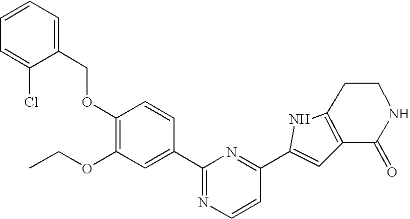 | 2-(2-{4-[(2-chlorobenzyl)oxy]-3-ethoxyphenyl}pyrimidin-4- yl)-1,5,6,7-tetrahydro-4H-pyrrolo[3,2-c]pyridin-4-one |
| 1262 | 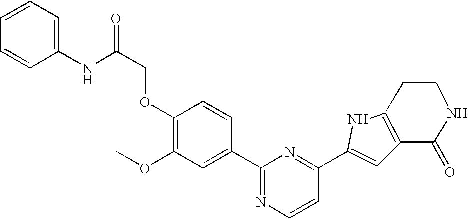 | 2-{2-methoxy-4-[4-(4-oxo-4,5,6,7-tetrahydro-1H- pyrrolo[3,2-c]pyridin-2-yl)pyrimidin-2-yl]phenoxy}-N- phenylacetamide |
| 1263 | 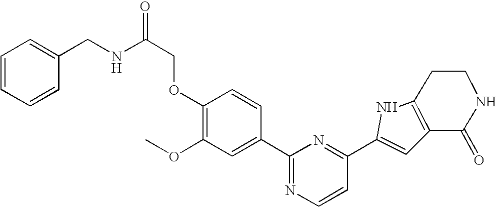 | N-benzyl-2-{2-methoxy-4-[4-(4-oxo-4,5,6,7-tetrahydro-1H- pyrrolo[3,2-c]pyridin-2-yl)pyrimidin-2- yl]phenoxy}acetamide |
| 1264 | 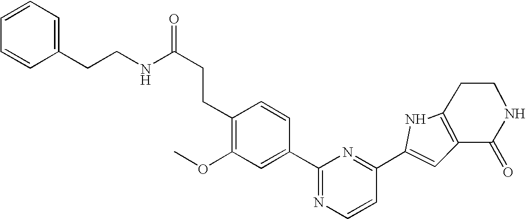 | 2-{2-methoxy-4-[4-(4-oxo-4,5,6,7-tetrahydro-1H- pyrrolo[3,2-c]pyridin-2-yl)pyrimidin-2-yl]phenoxy}-N-(2- phenylethyl)acetamide |
| 1265 | 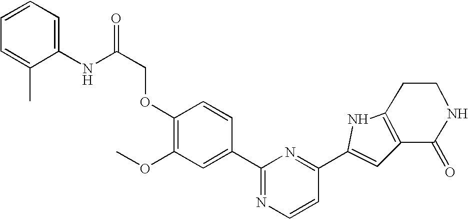 | 2-{2-methoxy-4-[4-(4-oxo-4,5,6,7-tetrahydro-1H- pyrrolo[3,2-c]pyridin-2-yl)pyrimidin-2-yl]phenoxy}-N-(2- methylphenyl)acetamide |
| 1266 | 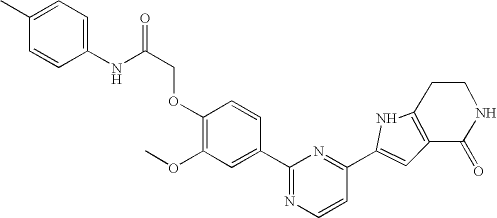 | 2-{2-methoxy-4-[4-(4-oxo-4,5,6,7-tetrahydro-1H- pyrrolo[3,2-c]pyridin-2-yl)pyrimidin-2-yl]phenoxy}-N-(4- methylphenyl)acetamide |
| 1267 | 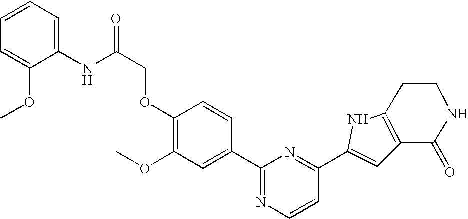 | 2-{2-methoxy-4-[4-(4-oxo-4,5,6,7-tetrahydro-1H- pyrrolo[3,2-c]pyridin-2-yl)pyrimidin-2-yl]phenoxy}-N-(2- methoxyphenyl)acetamide |
| 1268 | 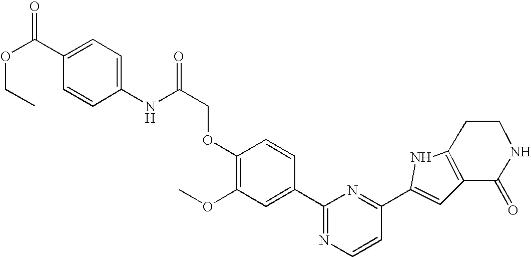 | ethyl 4-[({2-methoxy-4-[4-(4-oxo-4,5,6,7-tetrahydro-1H- pyrrolo[3,2-c]pyridin-2-yl)pyrimidin-2- yl]phenoxy}acetyl)amino]benzoate |
| 1269 | 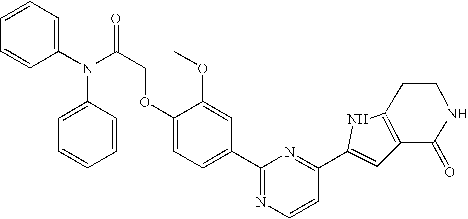 | 2-{2-methoxy-4-[4-(4-oxo-4,5,6,7-tetrahydro-1H- pyrrolo[3,2-c]pyridin-2-yl)pyrimidin-2-yl]phenoxy}-N,N- diphenylacetamide |
| 1270 | 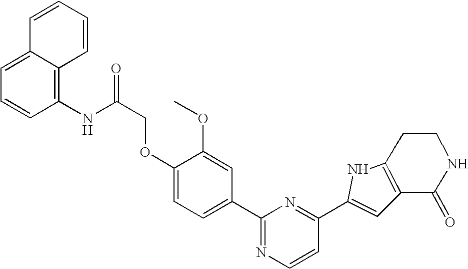 | 2-{2-methoxy-4-[4-(4-oxo-4,5,6,7-tetrahydro-1H- pyrrolo[3,2-c]pyridin-2-yl)pyrimidin-2-yl]phenoxy)-N-1- naphthylacetamide |
| 1271 | 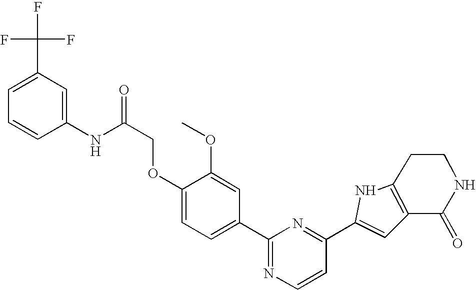 | 2-{2-methoxy-4-[4-(4-oxo-4,5,6,7-tetrahydro-1H- pyrrolo[3,2-c]pyridin-2-yl)pyrimidin-2-yl]phenoxy}-N-[3- (trifluoromethyl)phenyl]acetamide |
| 1272 | 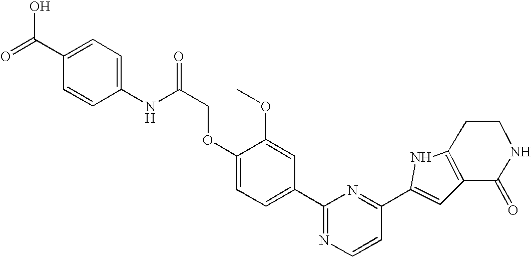 | 4-[({2-methoxy-4-[4-(4-oxo-4,5,6,7-tetrahydro-1H- pyrrolo[3,2-c]pyridin-2-yl)pyrimidin-2- yl]phenoxy}acetyl)amino]benzoic acid |
| 1273 | 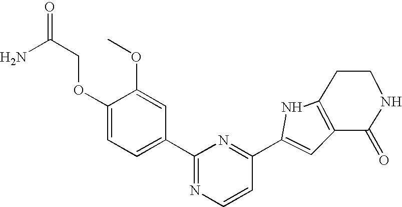 | 2-{2-methoxy-4-[4-(4-oxo-4,5,6,7-tetrahydro-1H- pyrrolo[3,2-c]pyridin-2-yl)pyrimidin-2- yl]phenoxy}acetamide |
| 1274 | 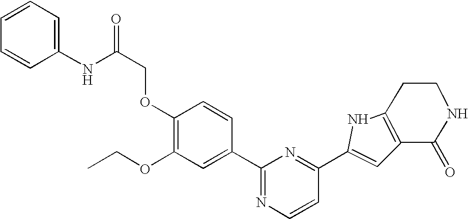 | 2-{2-ethoxy-4-[4-(4-oxo-4,5,6,7-tetrahydro-1H-pyrrolo[3,2- c]pyridin-2-yl)pyrimidin-2-yl]phenoxy}-N-phenylacetamide |
| 1275 | 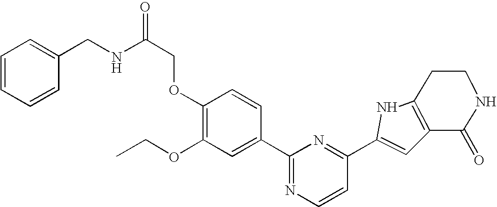 | N-benzyl-2-{2-ethoxy-4-[4-(4-oxo-4,5,6,7-tetrahydro-1H- pyrrolo[3,2-c]pyridin-2-yl)pyrimidin-2- yl]phenoxy}acetamide |
| 1276 | 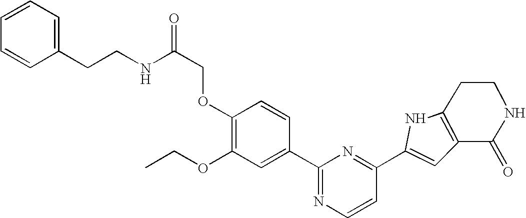 | 2-{2-ethoxy-4-[4-(4-oxo-4,5,6,7-tetrahydro-1H-pyrrolo[3,2- c]pyridin-2-yl)pyrimidin-2-yl]phenoxy}-N-(2- phenylethyl)acetamide |
| 1277 | 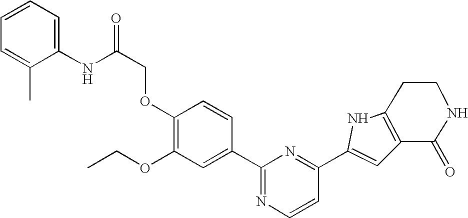 | 2-{2-ethoxy-4-[4-(4-oxo-4,5,6,7-tetrahydro-1H-pyrrolo[3,2- c]pyridin-2-yl)pyrimidin-2-yl]phenoxy}-N-(2- methylphenyl)acetamide |
| 1278 | 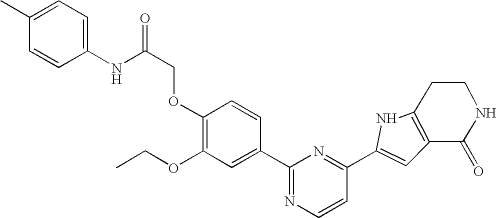 | 2-{2-ethoxy-4-[4-(4-oxo-4,5,6,7-tetrahydro-1H-pyrrolo[3,2- c]pyridin-2-yl)pyrimidin-2-yl]phenoxy}-N-(4- methylphenyl)acetamide |
| 1279 | 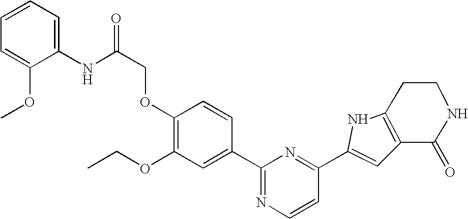 | 2-{2-ethoxy-4-[4-(4-oxo-4,5,6,7-tetrahydro-1H-pyrrolo[3,2- c]pyridin-2-yl)pyrimidin-2-yl]phenoxy}-N-(2- methoxyphenyl)acetamide |
| 1280 | 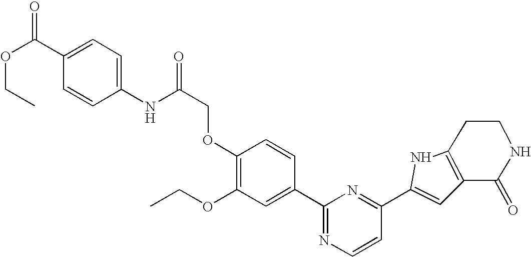 | ethyl 4-[({2-ethoxy-4-[4-(4-oxo-4,5,6,7-tetrahydro-1H- pyrrolo[3,2-c]pyridin-2-yl)pyrimidin-2- yl]phenoxy}acetyl)amino]benzoate |
| 1281 | 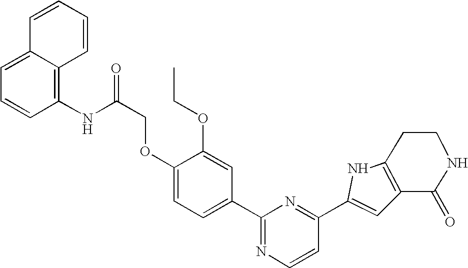 | 2-{2-ethoxy-4-[4-(4-oxo-4,5,6,7-tetrahydro-1H-pyrrolo[3,2- c]pyridin-2-yl)pyrimidin-2-yl]phenoxy}-N-1- naphthylacetamide |
| 1282 | 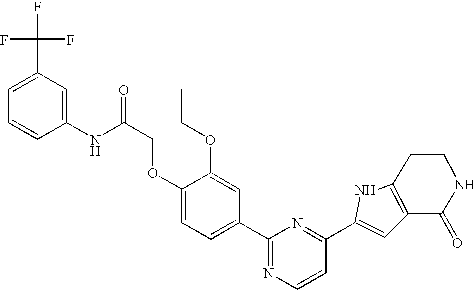 | 2-{2-ethoxy-4-[4-(4-oxo-4,5,6,7-tetrahydro-1H-pyrrolo[3,2- c]pyridin-2-yl)pyrimidin-2-yl]phenoxy}-N-[3- (trifluoromethyl)phenyl]acetamide |
| 1283 | 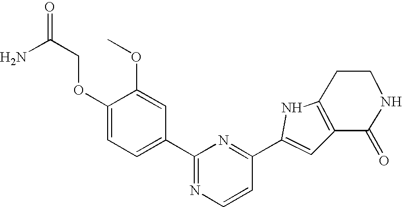 | 2-{2-ethoxy-4-[4-(4-oxo-4,5,6,7-tetrahydro-1H-pyrrolo[3,2- c]pyridin-2-yl)pyrimidin-2-yl]phenoxy}acetamide |
| 1284 | 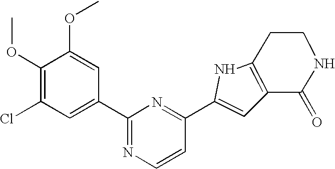 | 2-[2-(3-chloro-4,5-dimethoxyphenyl)pyrimidin-4-yl]-1,5,6,7- tetrahydro-4H-pyrrolo[3,2-c]pyridin-4-one |
| 1285 | 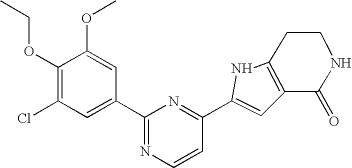 | 2-[2-(3-chloro-4-ethoxy-5-methoxyphenyl)pyrimidin-4-yl]- 1,5,6,7-tetrahydro-4H-pyrrolo[3,2-c]pyridin-4-one |
| 1286 | 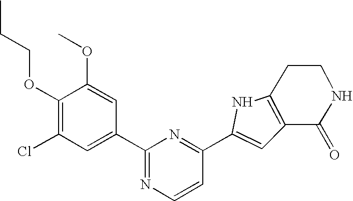 | 2-[2-(3-chloro-5-methoxy-4-propoxyphenyl)pyrimidin-4-yl]- 1,5,6,7-tetrahydro-4H-pyrrolo[3,2-c]pyridin-4-one |
| 1287 | 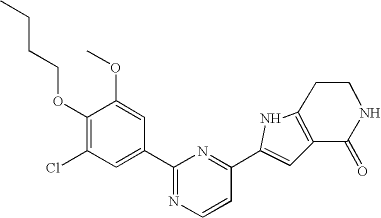 | 2-[2-(4-butoxy-3-chloro-5-methoxyphenyl)pyrimidin-4-yl]- 1,5,6,7-tetrahydro-4H-pyrrolo[3,2-c]pyridin-4-one |
| 1288 | 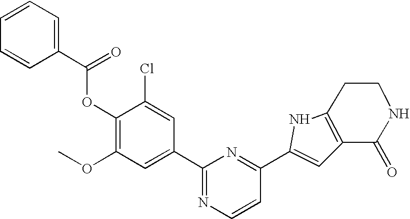 | 2-chloro-6-methoxy-4-[4-(4-oxo-4,5,6,7-tetrahydro-1H- pyrrolo[3,2-c]pyridin-2-yl)pyrimidin-2-yl]phenyl benzoate |
| 1289 | 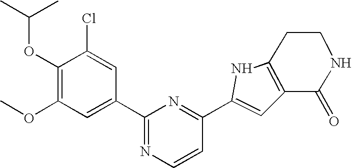 | 2-[2-(3-chloro-4-isopropoxy-5-methoxyphenyl)pyrimidin-4- yl]-1,5,6,7-tetrahydro-4H-pyrrolo[3,2-c]pyridin-4-one |
| 1290 | 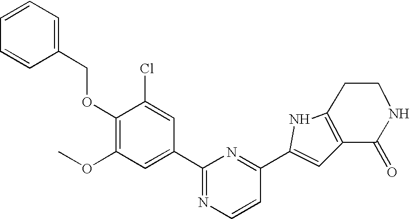 | 2-{2-[4-(benzyloxy)-3-chloro-5-methoxyphenyl]pyrimidin-4- yl}-1,5,6,7-tetrahydro-4H-pyrrolo[3,2-c]pyridin-4-one |
| 1291 | 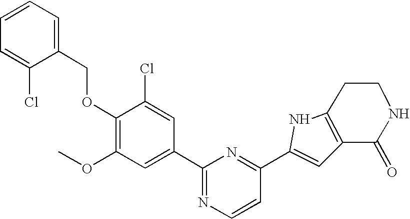 | 2-(2-{3-chloro-4-[(2-chIorobenzyl)oxy]-5- methoxyphenyl}pyrimidin-4-yl)-1,5,6,7-tetrahydro-4H- pyrrolo[3,2-c]pyridin-4-one |
| 1292 | 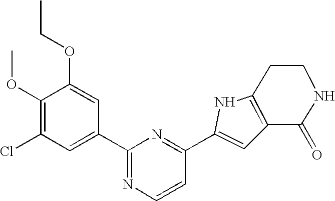 | 2-[2-(3-chloro-5-ethoxy-4-methoxyphenyl)pyrimidin-4-yl]- 1,5,6,7-tetrahydro-4H-pyrrolo[3,2-c]pyridin-4-one |
| 1293 | 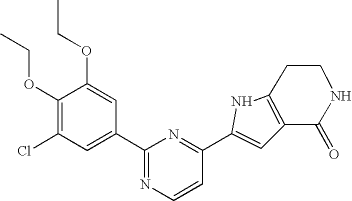 | 2-[2-(3-chloro-4,5-diethoxyphenyl)pyrimidin-4-yl]-1,5,6,7- tetrahydro-4H-pyrrolo[3,2-c]pyridin-4-one |
| 1294 | 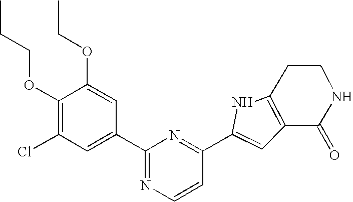 | 2-[2-(3-chloro-5-ethoxy-4-propoxyphenyl)pyrimidin-4-yl]- 1,5,6,7-tetrahydro-4H-pyrrolo[3,2-c]pyridin-4-one |
| 1295 | 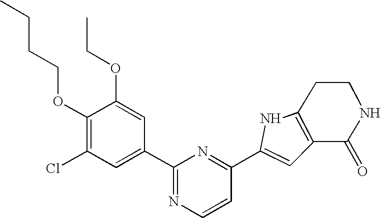 | 2-[2-(4-butoxy-3-chloro-5-ethoxyphenyl)pyrimidin-4-yl]- 1,5,6,7-tetrahydro-4H-pyrrolo[3,2-c]pyridin-4-one |
| 1296 | 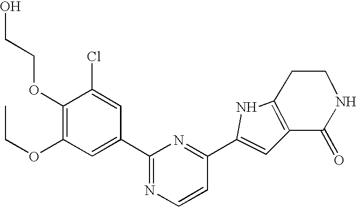 | 2-{2-[3-chloro-5-ethoxy-4-(2- hydroxyethoxy)phenyl]pyrimidin-4-yl}-1,5,6,7-tetrahydro- 4H-pyrrolo[3,2-c]pyridin-4-one |
| 1297 | 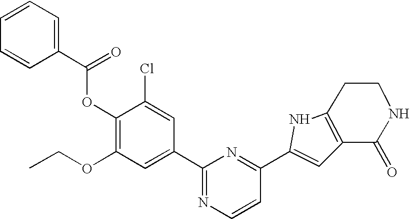 | 2-chloro-6-ethoxy-4-[4-(4-oxo-4,5,6,7-tetrahydro-1H- pyrrolo[3,2-c]pyridin-2-yl)pyrimidin-2-yl]phenyl benzoate |
| 1298 | 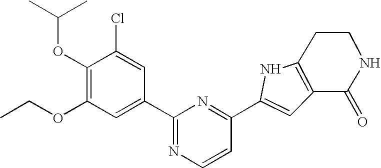 | 2-[2-(3-chloro-5-ethoxy-4-isopropoxyphenyl)pyrimidin-4- yl]-1,5,6,7-tetrahydro-4H-pyrrolo[3,2-c]pyridin-4-one |
| 1299 | 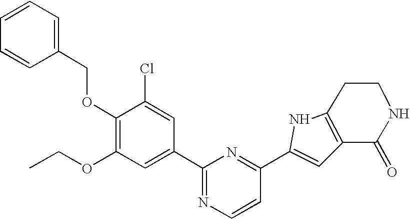 | 2-{2-[4-(benzyloxy)-3-chloro-5-ethoxyphenyl]pyrimidin-4- yl}-1,5,6,7-tetrahydro-4H-pyrrolo[3,2-c]pyridin-4-one |
| 1300 | 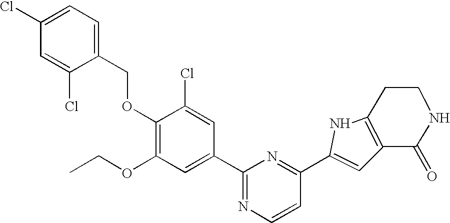 | 2-(2-{3-chloro-4-[(2,4-dichlorobenzyl)oxy]-5- ethoxyphenyl}pyrimidin-4-yl)-1,5,6,7-tetrahydro-4H- pyrrolo[3,2-c]pyridin-4-one |
| 1301 | 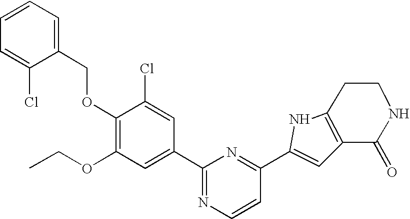 | 2-(2-{3-chloro-4-[(2-chlorobenzyl)oxy]-5- ethoxyphenyl]pyrimidin-4-yl)-1,5,6,7-tetrahydro-4H- pyrrolo[3,2-c]pyridin-4-one |
| 1302 | 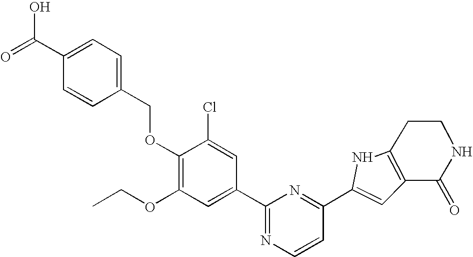 | 4-({2-chloro-6-ethoxy-4-[4-(4-oxo-4,5,6,7-tetrahydro-1H- pyrrolo[3,2-c]pyridin-2-yl)pyrimidin-2- yl]phenoxy}methyl)benzoic acid |
| 1303 | 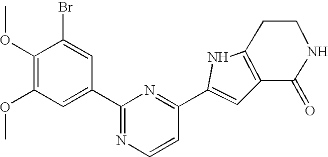 | 2-[2-(3-bromo-4,5-dimethoxyphenyl)pyrimidin-4-yl]- 1,5,6,7-tetrahydro-4H-pyrrolo[3,2-c]pyridin-4-one |
| 1304 | 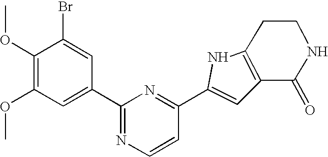 | 2-[2-(3-bromo-4-ethoxy-5-methoxyphenyl)pyrimidin-4-yl]- 1,5,6,7-tetrahydro-4H-pyrrolo[3,2-c]pyridin-4-one |
| 1305 | 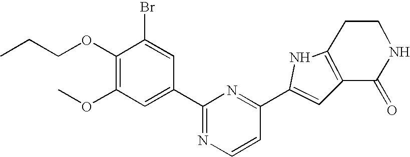 | 2-[2-(3-bromo-5-methoxy-4-propoxyphenyl)pyrimidin-4-yl]1,5,6,7-tetrahydro-4H-pyrrolo[3,2-c]pyridin-4-one |
| 1306 | 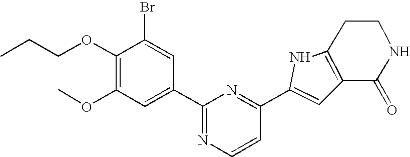 | 2-[2-(3-bromo-4-butoxy-5-methoxyphenyl)pyrimidin-4-yl]- 1,5,6,7-tetrahydro-4H-pyrrolo[3,2-c]pyridin-4-one |
| 1307 | 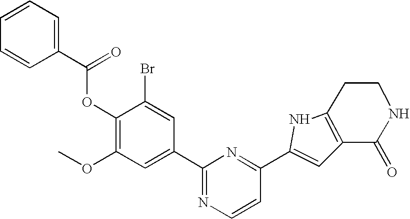 | 2-bromo-6-methoxy-4-[4-(4-oxo-4,5,6,7-tetrahydro-1H- pyrrolo[3,2-c]pyridin-2-yl)pyrimidin-2-yl]phenyl benzoate |
| 1308 | 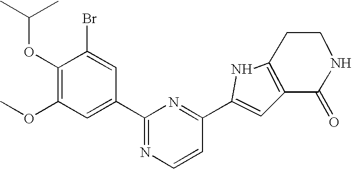 | 2-[2-(3-bromo-4-isopropoxy-5-methoxyphenyl)pyrimidin-4- yl]-1,56,7-tetrahydro-4H-pyrrolo[3,2-c]pyridin-4-one |
| 1309 | 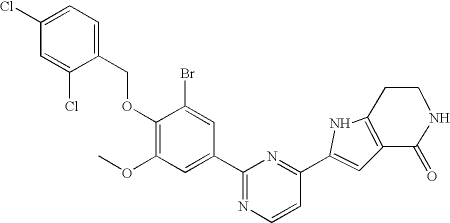 | 2-(2-{3-bromo-4-[(2,4-dichlorobenzyl)oxy]-5- methoxyphenyl}pyrimidin-4-yl)-1,5,6,7-tetrahydro-4H- pyrrolo[3,2-c]pyridin-4-one |
| 1310 | 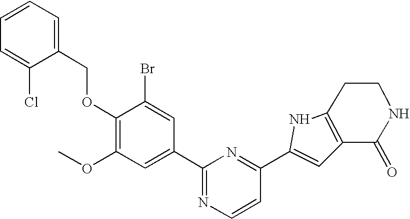 | 2-(2-{3-bromo-4-[(2-chlorobenzyl)oxy]-5- methoxyphenyl}pyrimidin-4-yl)-1,5,6,7-tetrahydro-4H- pyrrolo[3,2-c]pyridin-4-one |
| 1311 | 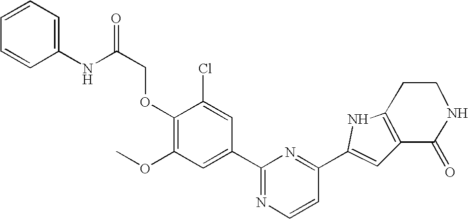 | 2-{2-chloro-6-methoxy-4-[4-(4-oxo-4,5,6,7-tetrahydro-1H- pyrrolo[3,2-c]pyridin-2-yl)pyrimidin-2-yl]phenoxy}-N- phenylacetamide |
| 1312 | 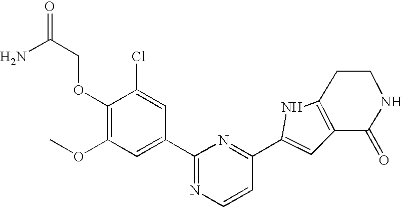 | 2-{2-chloro-6-methoxy-4-[4-(4-oxo-4,5,6,7-tetrahydro-1H- pyrrolo[3,2-c]pyridin-2-yl)pyrimidin-2- yl]phenoxylacetamide |
| 1313 | 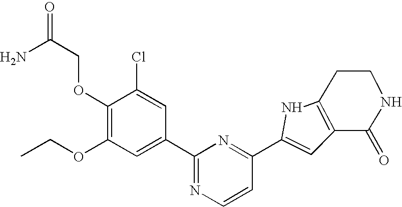 | 2-{2-chloro-6-ethoxy-4-[4-(4-oxo-4,5,6,7-tetrahydro-1H- pyrrolo[3,2-c]pyridin-2-yl)pyrimidin-2- yl]phenoxy}acetamide |
| 1314 | 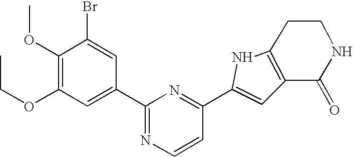 | 2-[2-(3-bromo-5-ethoxy-4-methoxyphenyl)pyrimidin-4-yl]- 1,5,6,7-tetrahydro-4H-pyrrolo[3,2-c]pyridin-4-one |
| 1315 | 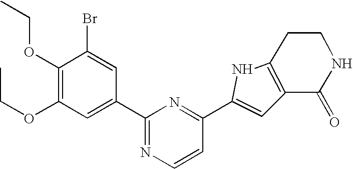 | 2-[2-(3-bromo-4,5-diethoxyphenyl)pyrimidin-4-yl]-1,5,6,7- tetrahydro-4H-pyrrolo[3,2-c]pyridin-4-one |
| 1316 | 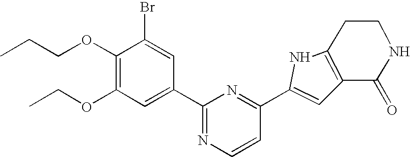 | 2-[2-(3-bromo-5-ethoxy-4-propoxyphenyl)pyrimidin-4-yl]- 1,5,6,7-tetrahydro-4H-pyrrolo[3,2-c]pyridin-4-one |
| 1317 | 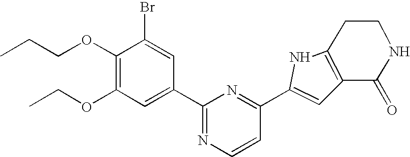 | 2-[2-(3-bromo-4-butoxy-5-ethoxyphenyl)pyrimidin-4-yl]- 1,5,6,7-tetrahydro-4H-pyrrolo[3,2-c]pyridin-4-one |
| 1318 | 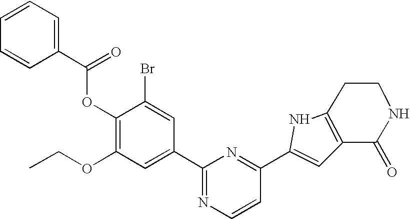 | 2-bromo-6-ethoxy-4-[4-(4-oxo-4,5,6,7-tetrahydro-1H- pyrrolo[3,2-c]pyridin-2-yl)pyrimidin-2-yl]phenyl benzoate |
| 1319 | 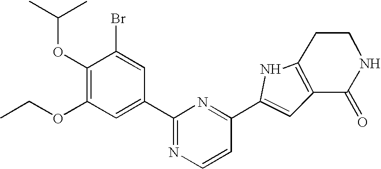 | 2-[2-(3-bromo-5-ethoxy-4-isopropoxyphenyl)pyrimidin-4- yl]-1,5,6,7-tetrahydro-4H-pyrrolo[3,2-c]pyridin-4-one |
| 1320 | 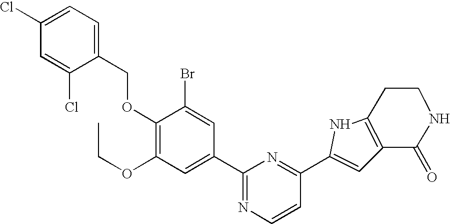 | 2-(2-{3-bromo-4-[(2,4-dichlorobenzyl)oxy]-5- ethoxyphenyl}pyrimidin-4-yl)-1,5,6,7-tetrahydro-4H- pyrrolo[3,2-c]pyridin-4-one |
| 1321 | 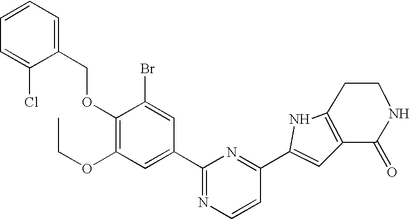 | 2-(2-{3-bromo-4-[(2-chlorobenzyl)oxy]-5- ethoxyphenyl}pyrimidin-4-yl)-1,5,6,7-tetrahydro-4H- pyrrolo[3,2-c]pyridin-4-one |
| 1322 | 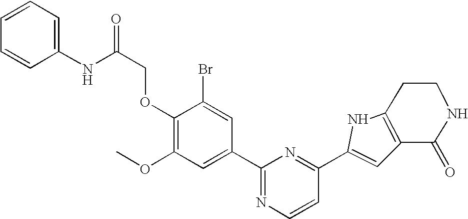 | 2-{2-bromo-6-methoxy-4-[4-(4-oxo-4,5,6,7-tetrahydro-1H- pyrrolo[3,2-c]pyridin-2-yl)pyrimidin-2-yl]phenoxy}-N- phenylacetamide |
| 1323 | 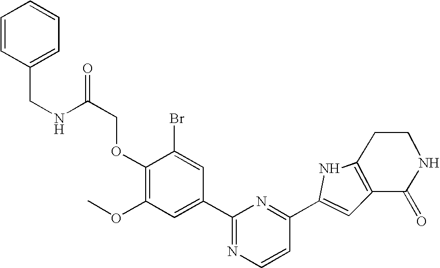 | N-benzyl-2-{2-bromo-6-methoxy-4-[4-(4-oxo-4,5,6,7- tetrahydro-1H-pyrrolo[3,2-c]pyridin-2-yl)pyrimidin-2- yl]phenoxy}acetamide |
| 1324 | 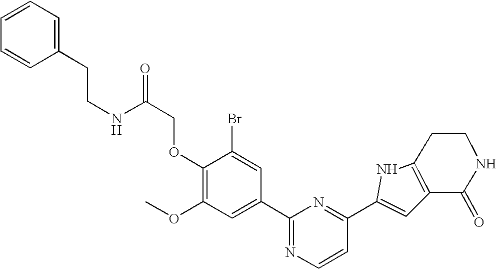 | 2-{2-bromo-6-rnethoxy-4-[4-(4-oxo-4,5,6,7-tetrahydro-1H- phenylethyl)acetamide |
| 1325 | 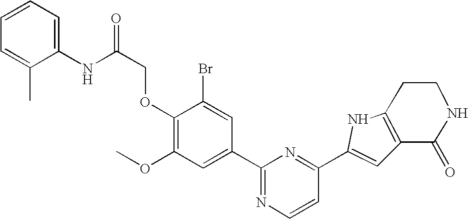 | 2-{2-bromo-6-methoxy-4-[4-(4-oxo-4,5,6,7-tetrahydro-1H- pyrrolo[3,2-cJpyrldln-2-yl)pyrimidin-2-yl]phenoxy}-N-(2- methylphenyl)acetamide |
| 1326 | 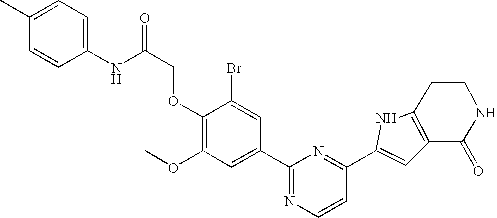 | 2-{2-bromo-6-methoxy-4-[4-(4-oxo-4,5,6,7-tetrahydro-1H- pyrrolo[3,2-c]pyridin-2-yl)pyrimidin-2-yl]phenoxy}-N-(4- methylphenyl)acetamide |
| 1327 | 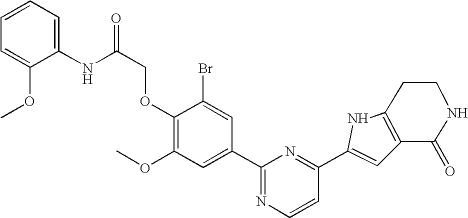 | 2-{2-bromo-6-methoxy-4-[4-(4-oxo-4,5,6,7-tetrahydro-1H- pyrrolo[3,2-c]pyridin-2-yl)pyrimidin-2-yl]phenoxy}-N-(2- methoxyphenyl)acetamide |
| 1328 | 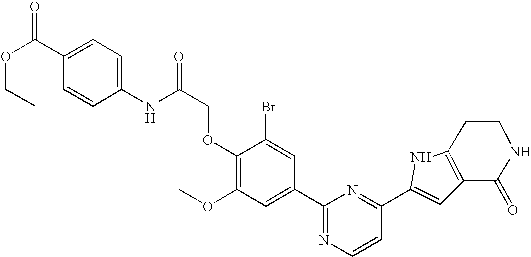 | ethyl 4-[({2-bromo-6-methoxy-4-[4-(4-oxo-4,5,6,7- tetrahydro-1H-pyrrolo[3,2-c]pyridin-2-yl)pyrimidin-2- yl]phenoxy}acetyl)amino]benzoate |
| 1329 | 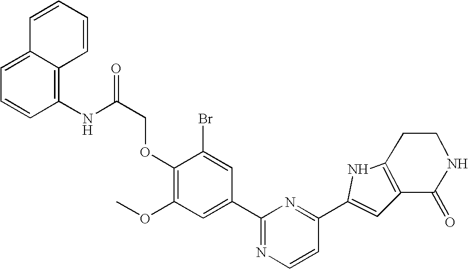 | 2-{2-bromo-6-methoxy-4-[4-(4-oxo-4,5,6,7-tetrahydro-1H- pyrrolo[3,2-c]pyridin-2-yl)pyrimidin-2-yl]phenoxy}-N-1- naphthylacetamide |
| 1330 | 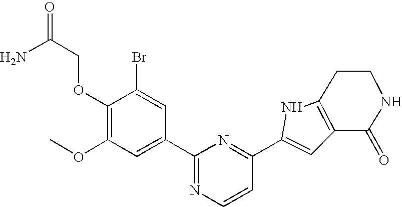 | 2-{2-bromo-6-methoxy-4-[4-(4-oxo-4,5,6,7-tetrahydro-1H- pyrrolo[3,2-c]pyridin-2-yl)pyrimidin-2- yl]phenoxylacetamide |
| 1331 | 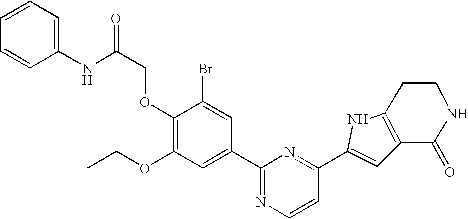 | 2-{2-bromo-6-ethoxy-4-[4-(4-oxo-4,5,6,7-tetrahydro-1H- pyrrolo[3,2-c]pyridin-2-yl)pyrimidin-2-yl]phenoxy}-N- phenylacetamide |
| 1332 | 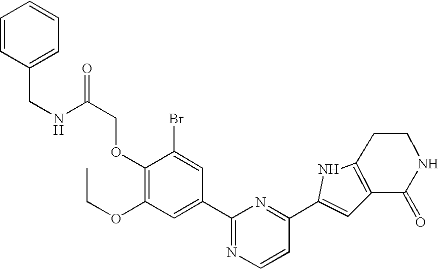 | N-benzyl-2-{2-bromo-6-ethoxy-4-[4-(4-oxo-4,5,6,7- tetrahydro-1H-pyrrolo[3,2-c]pyrdin-2-yl)pynmidin-2- yl]phenoxy}acetamide |
| 1333 | 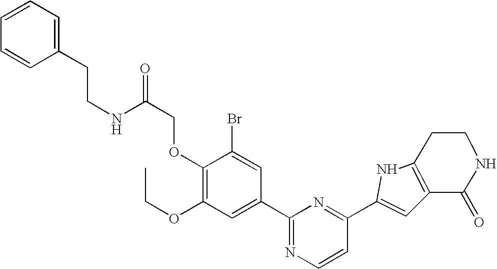 | 2-{2-bromo-6-ethoxy-4-[4-(4-oxo-4,5,6,7-tetrahydro-1H- pyrrolo[3,2-c]pyridin-2-yl)pyrimidin-2-yl]phenoxy}-N-(2- phenylethyl)acetamide |
| 1334 | 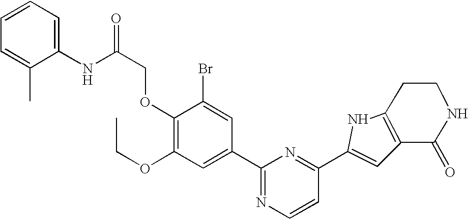 | 2-{2-bromo-6-ethoxy-4-[4-(4-oxo-4,5,6,7-tetrahydro-1H- pyrrolo[3,2-c]pyridin-2-yl)pyrimidin-2-yl]phenoxy}-N-(2- methylphenyl)acetamide |
| 1335 | 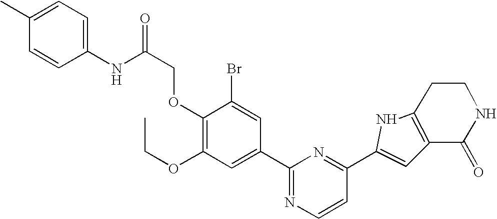 | 2-{2-bromo-6-ethoxy-4-[4-(4-oxo-4,5,6,7-tetrahydro-1H- pyrrolo[3,2-c]pyridin-2-yl)pyrimidin-2-yl]phenoxy}-N-(4- methylphenyl)acetamide |
| 1336 | 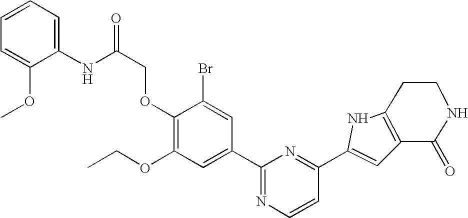 | 2-{2-bromo-6-ethoxy-4-[4-(4-oxo-4,5,6,7-tetrahydro-1H- pyrrolo[3,2-c]pyridin-2-yl)pyrimidin-2-yl]phenoxy}-N-(2- methoxyphenyl)acetamide |
| 1337 | 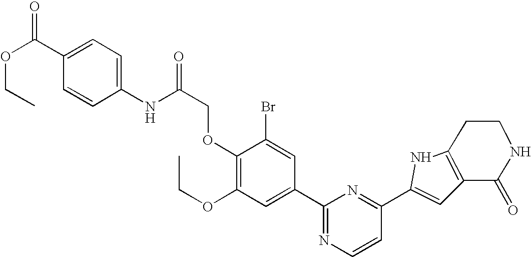 | ethyl 4-[({2-bromo-6-ethoxy-4-[4-(4-oxo-4,5,6,7-tetrahydro- 1H-pyrrolo[3,2-c]pyridin-2-yl)pyrimidin-2- yl]phenoxy}acetyl)amino]benzoate |
| 1338 | 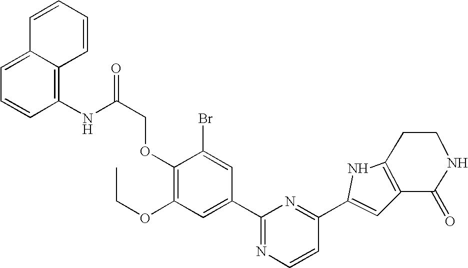 | 2-{2-bromo-6-ethoxy-4-[4-(4-oxo-4,5,6,7-tetrahydro-1H- pyrrolo[3,2-c]pyridin-2-yl)pyrimidin-2-yl]phenoxy}-N-1- naphthylacetamide |
| 1339 | 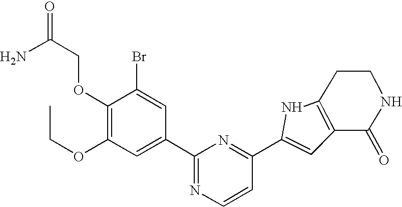 | 2-{2-bromo-6-ethoxy-4-[4-(4-oxo-4,5,6,7-tetrahydro-1H- pyrrolo[3,2-c]pyridin-2-yl)pyrimidin-2- yl]phenoxy}acetamide |
| 1340 | 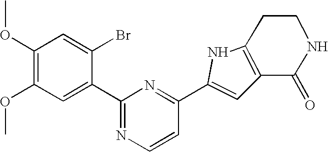 | 2-[2-(2-bromo-4,5-dimethoxyphenyl)pyrimidin-4-yl]- 1,5,6,7-tetrahydro-4H-pyrrolo[3,2-c]pyridin-4-one |
| 1341 | 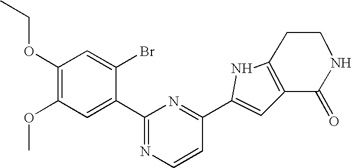 | 2-[2-(2-bromo-4-ethoxy-5-methoxyphenyl)pyrimidin-4-yl]- 1,5,6,7-tetrahydro-4H-pyrrolo[3,2-c]pyridin-4-one |
| 1342 | 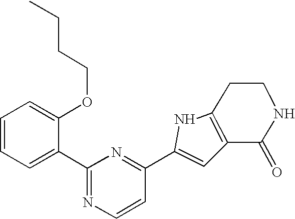 | 2-[2-(2-butoxyphenyl)pyrimidin-4-yl]-1,5,6,7-tetrahydro-4H- pyrrolo[3,2-c]pyridin-4-one |
| 1343 | 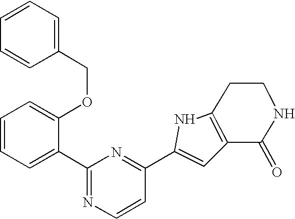 | 2-{2-[2-(benzyloxy)phenyl]pyrimidin-4-yl}-1,5,6,7- tetrahydro-4H-pyrrolo[3,2-c]pyridin-4-one |
| 1344 | 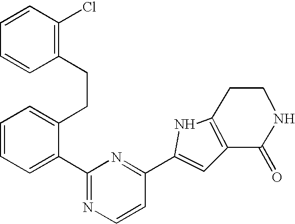 | 2-(2-{2-[(2-chlorobenzyl)oxy]phenyl}pyrimidin-4-yl)-1,5,6,7- tetrahydro-4H-pyrrolo[3,2-c]pyridin-4-one |
| 1345 | 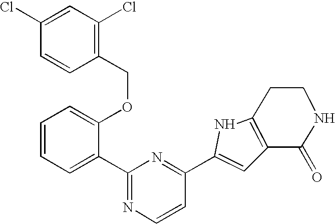 | 2-(2-{2-[(2,4-dichlorobenzyl)oxy]phenyl}pyrimidin-4-yl)- 1,5,6,7-tetrahydro-4H-pyrrolo[3,2-c]pyridin-4-one |
| 1346 | 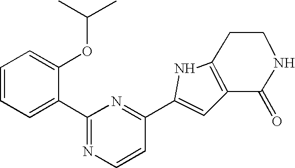 | 2-[2-(2-isopropoxyphenyl)pyrimidin-4-yl]-1,5,6,7- tetrahydro-4H-pyrrolo[3,2-c]pyridin-4-one |
| 1347 | 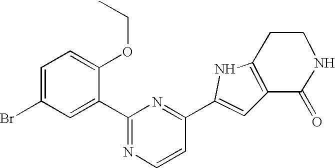 | 2-[2-(5-bromo-2-ethoxyphenyl)pyrimidin-4-yl]-1,5,6,7- tetrahydro-4H-pyrrolo[3,2-c]pyridin-4-one |
| 1348 | 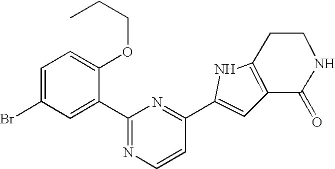 | 2-[2-(5-bromo-2-propoxyphenyl)pyrimidin-4-yl]-1,5,6,7- tetrahydro-4H-pyrrolo[3,2-c]pyridin-4-one |
| 1349 | 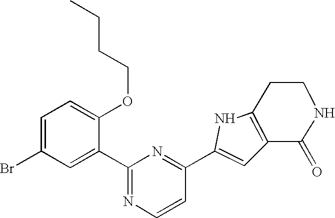 | 2-[2-(5-bromo-2-butoxyphenyl)pyrimidin-4-yl]-1,5,6,7- tetrahydro-4H-pyrrolo[3,2-c]pyridin-4-one |
| 1350 | 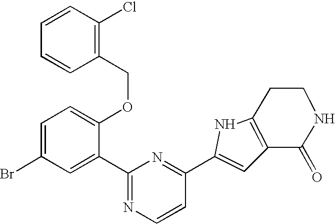 | 2-(2-{5-bromo-2-[(2-chlorobenzyl)oxy]phenyl]pyrimidin-4- yl)-1,5,6,7-tetrahydro-4H-pyrrolo[3,2-c]pyridin-4-one |
| 1351 | 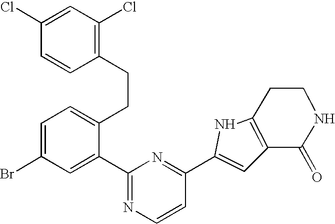 | 2-(2-{5-bromo-2-[(2,4-dichlorobenzyl)oxy]phenyl]pyrimidin- 4-yl)-1,5,6,7-tetrahydro-4H-pyrrolo[3,2-c]pyridin-4-one |
| 1352 | 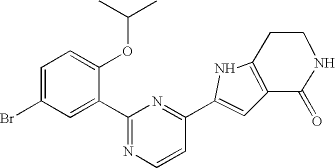 | 2-[2-(5-bromo-2-isopropoxyphenyl)pyrimidin-4-yl]-1,5,6,7- tetrahydro-4H-pyrrolo[3,2-c]pyridin-4-one |
| 1353 | 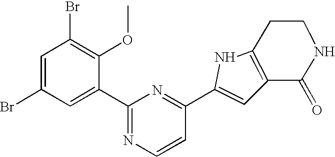 | 2-[2-(3,5-dibromo-2-methoxyphenyl)pyrimidin-4-yl]- 1,5,6,7-tetrahydro-4H-pyrrolo[3,2-c]pyridin-4-one |
| 1354 | 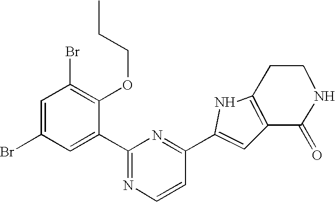 | 2-[2-(3,5-dibromo-2-propoxyphenyl)pyrimidin-4-yl]-1,5,6,7- tetrahydro-4H-pyrrolo[3,2-c]pyridin-4-one |
| 1355 | 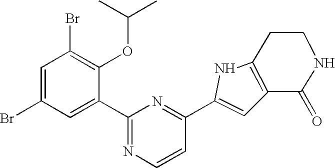 | 2-[2-(3,5-dibromo-2-isopropoxyphenyl)pyrimidin-4-yl]- 1,5,6,7-tetrahydro-4H-pyrrolo[3,2-c]pyridin-4-one |
| 1356 | 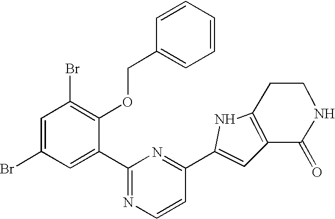 | 2-{2-[2-(benzyloxy)-3,5-dibromophenyl]pyrimidin-4-yl}- 1,5,6,7-tetrahydro-4H-pyrrolo[3,2-c]pyridin-4-one |
| 1357 | 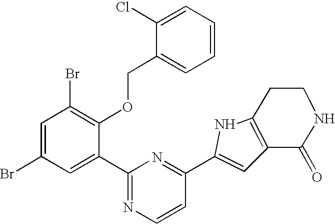 | 2-(2-{3,5-dibromo-2-[(2-chlorobenzyl)oxy]phenyl}pyrimidin- 4-yl)-1,5,6,7-tetrahydro-4H-pyrrolo[3,2-c]pyridin-4-one |
| 1358 | 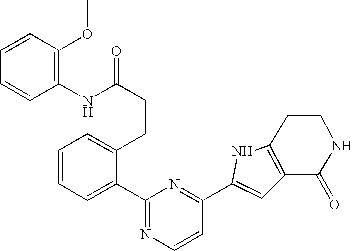 | N-(2-methoxyphenyl)-2-{2-[4-(4-oxo-4,5,6,7-tetrahydro-1H- pyrrolo[3,2-c]pyridin-2-yl)pyrimidin-2- yl]phenoxy}acetamide |
| 1359 | 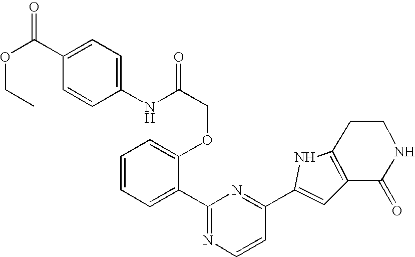 | ethyl 4-[({2-[4-(4-oxo-4,5,6,7-tetrahydro-1H-pyrrolo[3,2- c]pyridin-2-yl)pyrimidin-2- yl]phenoxy}acetyl)amino]benzoate |
| 1360 | 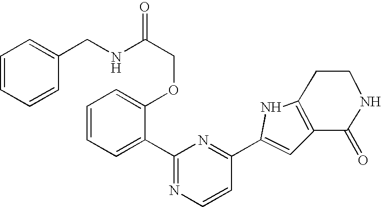 | N-benzyl-2-{2-[4-(4-oxo-4,5,6,7-tetrahydro-1H-pyrrolo[3,2- c]pyridin-2-yl)pyrimidin-2-yl]phenoxy}acetamide |
| 1361 | 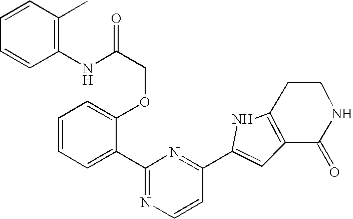 | N-(2-methylphenyl)-2-{2-[4-(4-oxo-4,5,6,7-tetrahydro-1H- pyrrolo[3,2-c]pyridin-2-yl)pyrimidin-2- yl]phenoxy}acetamide |
| 1362 | 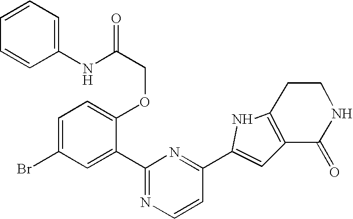 | 2-{4-bromo-2-[4-(4-oxo-4,5,6,7-tetrahydro-1H-pyrrolo[3,2- c]pyridin-2-yl)pyrimidin-2-yl]phenoxyl-N-phenylacetamide |
| 1363 | 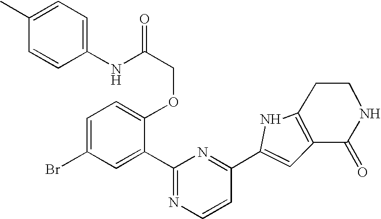 | 2-{4-bromo-2-[4-(4-oxo-4,5,6,7-tetrahydro-1H-pyrrolo[3,2- c]pyridin-2-yl)pyrimidin-2-yl]phenoxy}-N-(4- methylphenyl)acetamide |
| 1364 | 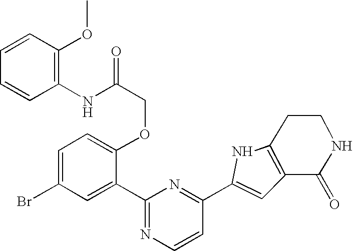 | 2-{4-bromo-2-[4-(4-oxo-4,5,6,7-tetrahydro-1H-pyrrolo[3,2- c]pyridin-2-yl)pyrimidin-2-yl]phenoxyl-N-(2- methoxyphenyl)acetamide |
| 1365 | 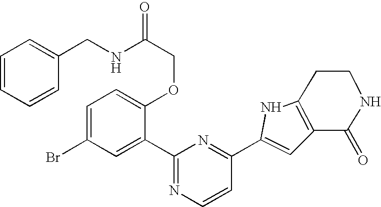 | N-benzyl-2-{4-bromo-2-[4-(4-oxo-4,5,6,7-tetrahydro-1H- pyrrolo[3,2-c]pyridin-2-yl)pyrimidin-2- yl]phenoxy}acetamide |
| 1366 | 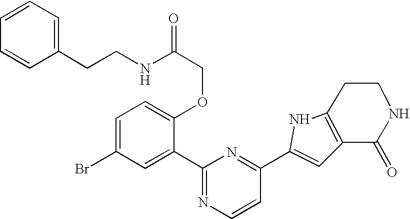 | 2-{4-bromo-2-[4-(4-oxo-4,5,6,7-tetrahydro-1H-pyrrolo[3,2- c]pyridin-2-yl)pyrimidin-2-yl]phenoxy}-N-(2- phenylethyl)acetamide |
| 1367 | 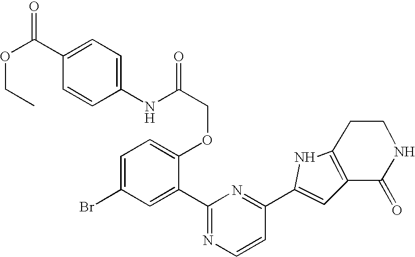 | ethyl 4-[({4-bromo-2-[4-(4-oxo-4,5,6,7-tetrahydro-1H- pyrrolo[3,2-c]pyridin-2-yl)pyrimidin-2- yl]phenoxy}acetyl)amino]benzoate |
| 1368 | 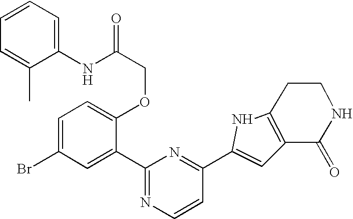 | 2-{4-bromo-2-[4-(4-oxo-4,5,6,7-tetrahydro-1H-pyrrolo[3,2- c]pyridin-2-yl)pyrimidin-2-yl]phenoxy}-N-(2- methylphenyl)acetamide |
| 1369 | 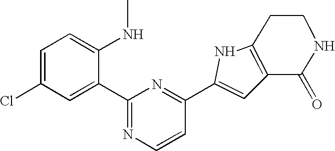 | 2-{2-[5-chloro-2-(methylamino)phenyl]pyrimidin-4-yl}- 1,5,6,7-tetrahydro-4H-pyrrolo[3,2-c]pyridin-4-one |
| 1370 | 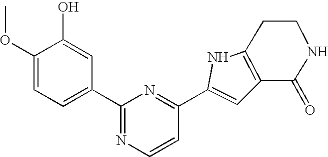 | 2-[2-(3-hydroxy-4-methoxyphenyl)pyrimidin-4-yl]-1,5,6,7- tetrahydro-4H-pyrrolo[3,2-c]pyridin-4-one |
| 1371 | 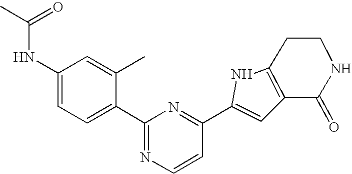 | N-{3-methyl-4-[4-(4-oxo-4,5,6,7-tetrahydro-1H-pyrrolo[3,2- c]pyridin-2-yl)pyrimidin-2-yl]phenyl}acetamide |
| 1372 | 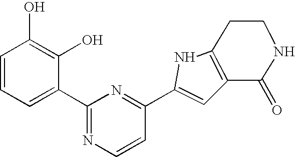 | 2-[2-(2,3-dihydroxyphenyl)pyrimidin-4-yl]-1 5,6,7- tetrahydro-4H-pyrrolo[3,2-c]pyridin-4-one |
| 1373 | 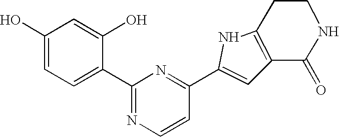 | 2-[2-(2,4-dihydroxyphenyl)pyrimidin-4-yl]-1,5,6,7- tetrahydro-4H-pyrrolo[3,2-c]pyridin-4-one |
| 1374 | 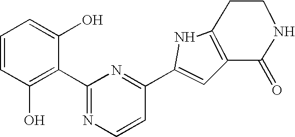 | 2-[2-(2,6-dihydroxyphenyl)pyrimidin-4-yl]-1,5,6,7- tetrahydro-4H-pyrrolo[3,2-c]pyridin-4-one |
| 1375 | 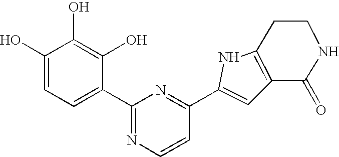 | 2-[2-(2,3,4-trihydroxyphenyl)pyrimidin-4-yl]-1,5,6,7- tetrahydro-4H-pyrrolo[3,2-c]pyridin-4-one |
| 1376 | 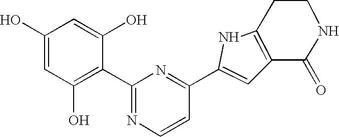 | 2-[2-(2,4,6-trihydroxyphenyl)pyrimidin-4-yl]-1,5,6,7- tetrahydro-4H-pyrrolo[3,2-c]pyridin-4-one |
| 1377 | 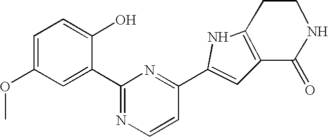 | 2-[2-(2-hydroxy-5-methoxyphenyl)pyrimidin-4-yl]-1,5,6,7- tetrahydro-4H-pyrrolo[3,2-c]pyridin-4-one |
| 1378 | 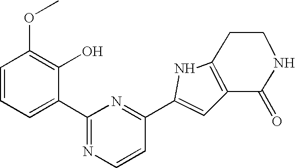 | 2-[2-(2-hydroxy-3-methoxyphenyl)pyrimidin-4-yl]-1,5,6,7- tetrahydro-4H-pyrrolo[3,2-c]pyridin-4-one |
| 1379 | 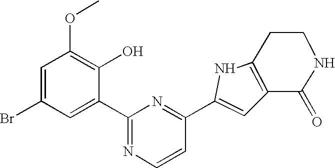 | 2-[2-(5-bromo-2-hydroxy-3-methoxyphenyl)pyrimidin-4-yl]- 1,5,6,7-tetrahydro-4H-pyrrolo[3,2-cfpyridin-4-one |
| 1380 | 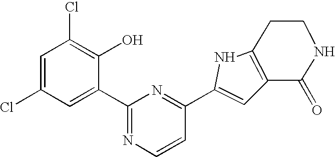 | 2-[2-(3,5-dichloro-2-hydroxyphenyl)pyrimidin-4-yl]-1,5,6,7- tetrahydro-4H-pyrrolo[3,2-c]pyridin-4-one |
| 1381 | 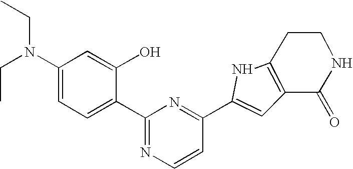 | 2-{2-[4-(diethylamino)-2-hydroxyphenyl]pyrimidin-4-yl}- 1,5,6,7-tetrahydro-4H-pyrrolo[3,2-c]pyridin-4-one |
| 1382 | 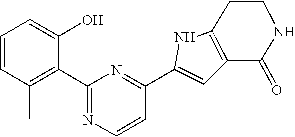 | 2-[2-(2-hydroxy-6-methylphenyl)pyrimidin-4-yl]-1,5,6,7- tetrahydro-4H-pyrrolo[3,2-c]pyridin-4-one |
| 1383 | 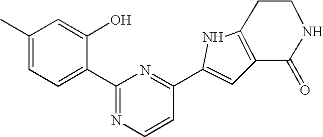 | 2-[2-(2-hydroxy-4-methylphenyl)pyrimidin-4-yl]-1,5,6,7- tetrahydro-4H-pyrrolo[3,2-c]pyridin-4-one |
| 1384 | 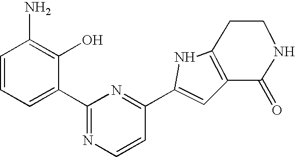 | 2-[2-(3-amino-2-hydroxyphenyl)pyrimidin-4-yl]-1,5,6,7- tetrahydro-4H-pyrrolo[3,2-c]pyridin-4-one |
| 1385 | 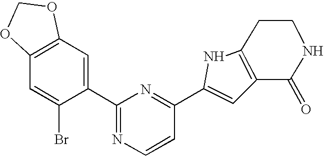 | 2-[2-(6-bromo-1,3-benzodioxol-5-yl)pyrimidin-4-yl]-1,5,6,7- tetrahydro-4H-pyrrolo[3,2-c]pyridin-4-one |
| 1386 | 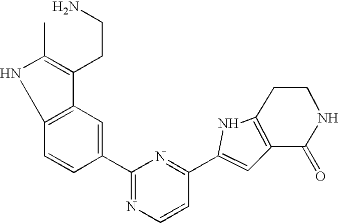 | 2-{2-[3-(2-aminoethyl)-2-methyl-1H-indol-5-yl]pyrimidin-4- yl}-1,5,6,7-tetrahydro-4H-pyrrolo[3,2-c]pyridin-4-one |
| 1387 | 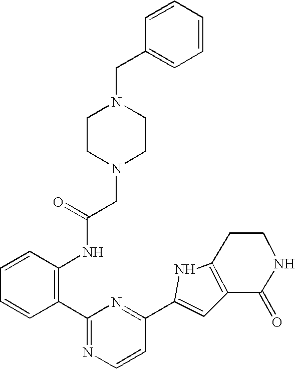 | 2-(4-benzylpiperazin-1-yl)-N-{2-[4-(4-oxo-4,5,6,7- tetrahydro-1H-pyrrolo[3,2-c]pyridin-2-yl)pyrimidin-2- yl]phenyl}acetamide |
| 1388 | 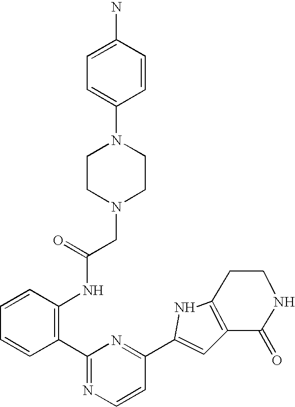 | 2-[4-(4-fluorophenyl)piperazin-1-yl]-N-{2-[4-(4-oxo-4,5,6,7- tetrahydro-1H-pyrrolo[3,2-c]pyridin-2-yl)pyrimidin-2- yl]phenyl}acetamide |
| 1389 | 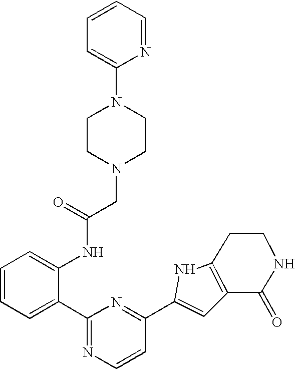 | N-{2-[4-(4-oxo-4,5,6,7-tetrahydro-1H-pyrrolo[3,2-c]pyridin- 2-yl)pyrimidin-2-yl]phenyl}-2-(4-pyridin-2-ylpiperazin-1- yl)acetamide |
| 1390 | 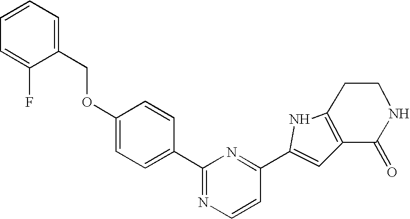 | 2-(2-{4-[(2-fluorobenzyl)oxy]phenyl}pyrimidin-4-yl)-1,5,6,7- tetrahydro-4H-pyrrolo[32-c]pyridin-4-one |
| 1391 | 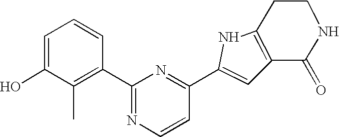 | 2-[2-(3-hydroxy-2-methylphenyl)pyrimidin-4-yl]-1,5,6,7- tetrahydro-4H-pyrrolo[3,2-c]pyridin-4-one |
| 1392 | 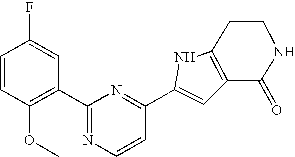 | 2-[2-(5-fluoro-2-methoxyphenyl)pyrimidin-4-yl]-1,5,6,7- tetrahydro-4H-pyrrolo[3,2-c]pyridin-4-one |
| 1393 | 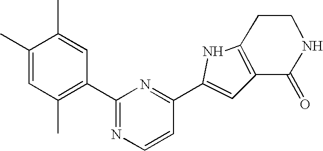 | 2-[2-(2,4,5-trimethylphenyl)pyrimidin-4-yl]-1,5,6,7- tetrahydro-4H-pyrrolo[3,2-c]pyridin-4-one |
| 1394 | 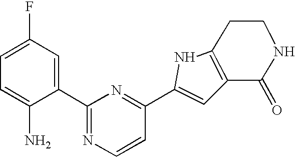 | 2-[2-(2-amino-5-fluorophenyl)pyrimidin-4-yl]-1,5,6,7- tetrahydro-4H-pyrrolo[3,2-c]pyridin-4-one |
| 1395 | 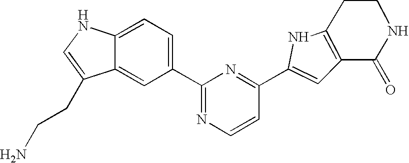 | 2-{2-[3-(2-aminoethyl)-1H-indol-5-yl]pyrimidin-4-yl}- 1,5,6,7-tetrahydro-4H-pyrrolo[3,2-c]pyridin-4-one |
| 1396 | 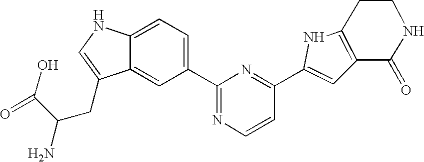 | 5-[4-(4-oxo-4,5,6,7-tetrahydro-1H-pyrrolo[3,2-c]pyridin-2- yl)pyrimidin-2-yl]tryptophan |
| 1397 | 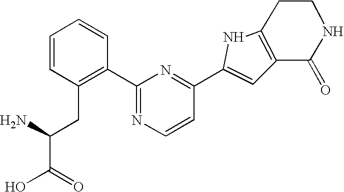 | 2-[4-(4-oxo-4,5,6,7-tetrahydro-1H-pyrrolo[3,2-c]pyridin-2- yl)pyrimidin-2-yl]-L-phenylalanine |
| 1398 | 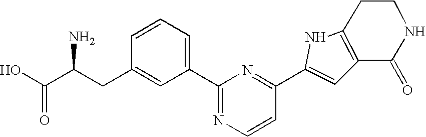 | 3-[4-(4-oxo-4,5,6,7-tetrahydro-1H-pyrrolo[3,2-c]pyridin-2- yl)pyrimidin-2-yl]-L-phenylalanine |
| 1399 | 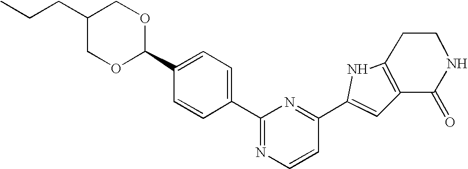 | 2-{2-[4-(5-propyl-1,3-dioxan-2-yl)phenyl]pyrimidin-4-yl)- 1,5,6,7-tetrahydro-4H-pyrrolo[3,2-c]pyridin-4-one |
| 1400 | 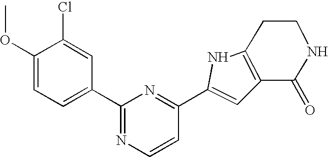 | 2-[2-(3-chloro-4-methoxyphenyl)pyrimidin-4-yl]-1,5,6,7- tetrahydro-4H-pyrrolo[3,2-c]pyridin-4-one |
| 1401 | 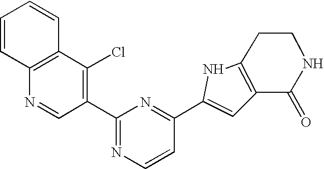 | 2-[2-(4-chloroquinoiin-3-yl)pyrimidin-4-yl]-1,5,6,7- tetrahydro-4H-pyrrolo[3,2-c]pyridin-4-one |
| 1402 | 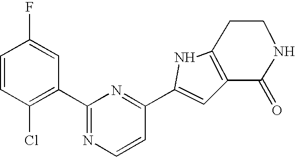 | 2-[2-(2-chloro-5-fluorophenyl)pyrimidin-4-yl]-1,5,6,7- tetrahydro-4H-pyrrolo[3,2-c]pyridin-4-one |
| 1403 | 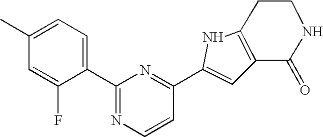 | 2-[2-(2-fluoro-4-methylphenyl)pyrimidin-4-yl]-1,5,6,7- tetrahydro-4H-pyrrolo[3,2-c]pyridin-4-one |
| 1404 | 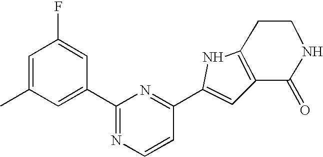 | 2-[2-(3-fluoro-5-methylphenyl)pyrimidin-4-yl]-1,5,6,7- tetrahydro-4H-pyrrolo[3,2-c]pyridin-4-one |
| 1405 | 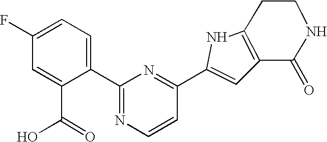 | 5-fluoro-2-[4-(4-oxo-4,5,6,7-tetrahydro-1H-pyrrolo[3,2- c]pyridin-2-yl)pyrimidin-2-yl]benzoic acid |
| 1406 | 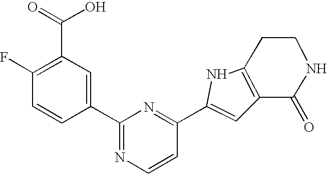 | 2-fluoro-5-[4-(4-oxo-4,5,6,7-tetrahydro-1H-pyrrolo[3,2- c]pyridin-2-yl)pyrimidin-2-yl]benzoic acid |
| 1407 | 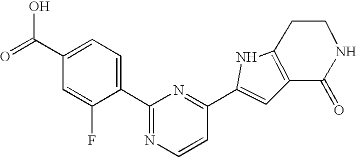 | 3-fluoro-4-[4-(4-oxo-4,5,6,7-tetrahydro-1H-pyrrolo[3,2- c]pyridin-2-yl)pyrimidin-2-yl]benzoic acid |
| 1408 | 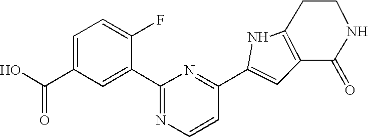 | 4-fluoro-3-[4-(4-oxo-4,5,6,7-tetrahydro-1H-pyrrolo[3,2- c]pyridin-2-yl)pyrimidin-2-yl]benzoic acid |
| 1409 | 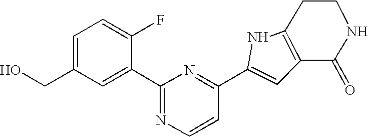 | 2-{2-[2-fluoro-5-(hydroxymethyl)phenyl]pyrimidin-4-yl}- 1,5,6,7-tetrahydro-4H-pyrrolo[3,2-c]pyridin-4-one |
| 1410 | 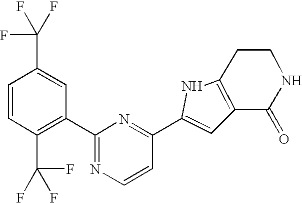 | 2-{2-[2,5-bis(trifluoromethyl)phenyl]pyrimidin-4-yl)-1,5,6,7- tetrahydro-4H-pyrrolo[3,2-c]pyridin-4-one |
| 1411 | 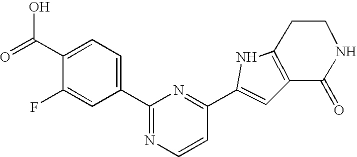 | 2-fluoro-4-[4-(4-oxo-4,5,6,7-tetrahydro-1H-pyrrolo[3,2- c]pyridin-2-yl)pyrimidin-2-yl]benzoic acid |
| 1412 | 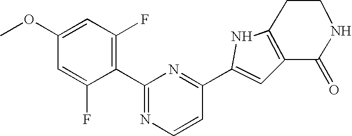 | 2-[2-(2,6-difluoro-4-methoxyphenyl)pyrimidin-4-yl]-1,5,6,7- tetrahydro-4H-pyrrolo[3,2-c]pyridin-4-one |
| 1413 | 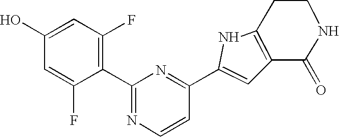 | 2-[2-(2,6-difluoro-4-hydroxyphenyl)pyrimidin-4-yl]-1,5,6,7- tetrahydro-4H-pyrrolo[3,2-c]pyridin-4-one |
| 1414 | 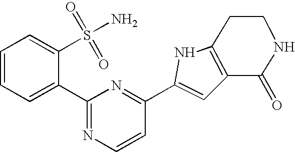 | 2-[4-(4-oxo-4,5,6,7-tetrahydro-1H-pyrrolo[3,2-c]pyridin-2- yl)pyrimidin-2-yl]benzenesulfonamide |
| 1415 | 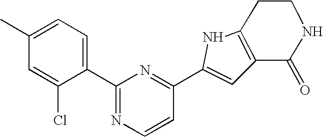 | 2-[2-(2-chloro-4-methylphenyl)pyrimidin-4-yl]-1,5,6,7- tetrahydro-4H-pyrrolo[3,2-c]pyridin-4-one |
| 1416 | 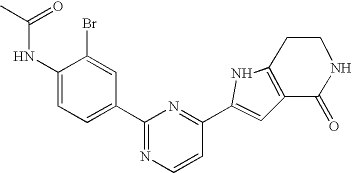 | N-{2-bromo-4-[4-(4-oxo-4,5,6,7-tetrahydro-1H-pyrrolo[3,2- c]pyridin-2-yl)pyrimidin-2-yl]phenyl}acetamide |
| 1417 | 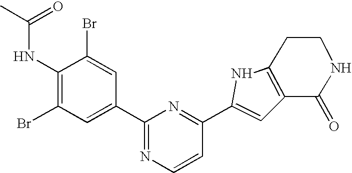 | N-{2,6-dibromo-4-[4-(4-oxo-4,5,6,7-tetrahydro-1H- pyrrolo[32-c]pyridin-2-yl)pyrimidin-2-yl]phenyl}acetamide |
| 1418 | 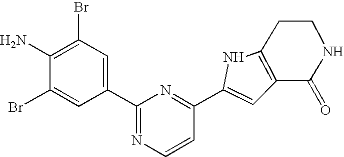 | 2-[2-(4-amino-3,5-dibromophenyl)pyrimidin-4-yl]-1,5,6,7- tetrahydro-4H-pyrrolo[3,2-c]pyridin-4-one |
| 1419 | 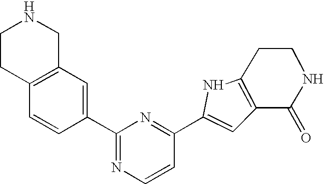 | 2-[2-(1,2,3,4-tetrahydroisoquinolin-7-yl)pyrimidin-4-yl]- 1,5,6,7-tetrahydro-4H-pyrrolo[3,2-c]pyridin-4-one |
| 1420 | 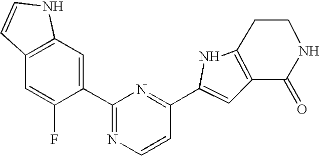 | 2-[2-(5-fluoro-1H-indol-6-yl)pyrimidin-4-yl]-1,5,6,7- tetrahydro-4H-pyrrolo[3,2-c]pyridin-4-one |
| 1421 | 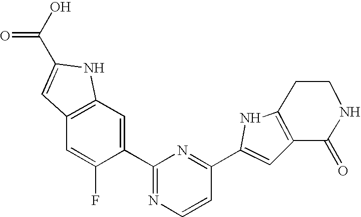 | 5-fluoro-6-[4-(4-oxo-4,5,6,7-tetrahydro-1H-pyrrolo[3,2- c]pyridin-2-yl)pyrimidin-2-yl]-1H-indole-2-carboxylic acid |
| 1422 | 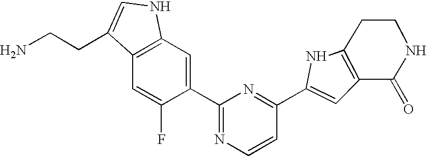 | 2-{2-[3-(2-aminoethyl)-5-fluoro-1H-indol-6-yl]pyrimidin-4- yl}-1,5,6,7-tetrahydro-4H-pyrrolo[3,2-c]pyridin-4-one |
| 1423 | 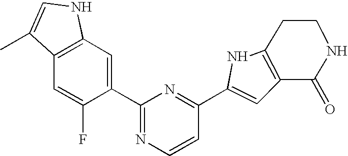 | 2-[2-(5-fluoro-3-methyl-1H-indol-6-yl)pyrimidin-4-yl]- 1,5,6,7-tetrahydro-4H-pyrrolo[3,2-c]pyridin-4-one |
| 1424 | 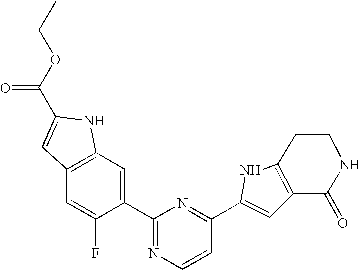 | ethyl 5-fluoro-6-[4-(4-oxo-4,5,6,7-tetrahydro-1H- pyrrolo[3,2-c]pyridin-2-yl)pyrimidin-2-yl]-1H-indole-2- carboxylate |
| 1425 | 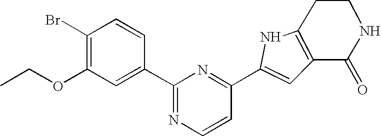 | 2-[2-(4-bromo-3-ethoxyphenyl)pyrimidin-4-yl]-1,5,6,7- tetrahydro-4H-pyrrolo[3,2-c]pyridin-4-one |
| 1426 | 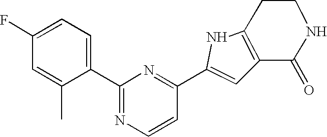 | 2-[2-(4-fluoro-2-methylphenyl)pyrimidin-4-yl]-1,5,6,7- tetrahydro-4H-pyrrolo[3,2-c]pyridin-4-one |
| 1427 | 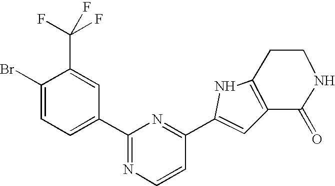 | 2-{2-[4-bromo-3-(trifluoromethyl)phenyl]pyrimidin-4-yl}- 1,5,6,7-tetrahydro-4H-pyrrolo[3,2-c]pyridin-4-one |
| 1428 | 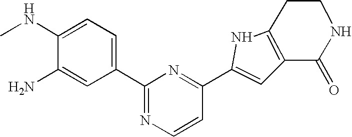 | 2-{2-[3-amino-4-(methylamino)phenyl]pyrimidin-4-yl}- 1,5,6,7-tetrahydro-4H-pyrrolo[3,2-c]pyridin-4-one |
| 1429 | 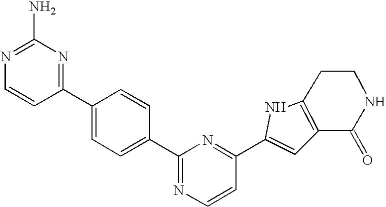 | 2-{2-[4-(2-aminopyrimidin-4-yl)phenyl]pyrimidin-4-yl}- 1,5,6,7-tetrahydro-4H-pyrrolo[3,2-c]pyridin-4-one |
| 1430 | 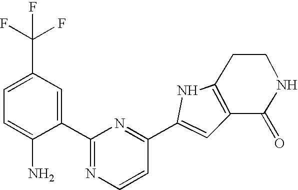 | 2-{2-[2-amino-5-(trifluoromethyl)phenyl]pyrimidin-4-yl}- 1,5,6,7-tetrahydro-4H-pyrrolo[3,2-c]pyridin-4-one |
| 1431 | 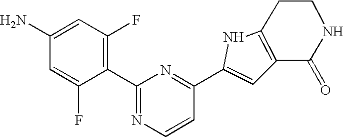 | 2-[2-(4-amino-2,6-difluorophenyl)pyrimidin-4-yl]-1,5,6,7- tetrahydro-4H-pyrrolo[3,2-c]pyridin-4-one |
| 1432 | 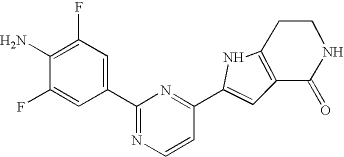 | 2-[2-(4-amino-3,5-difluorophenyl)pyrimidin-4-yl]-1,5,6,7- tetrahydro-4H-pyrrolo[3,2-c]pyridin-4-one |
| 1433 | 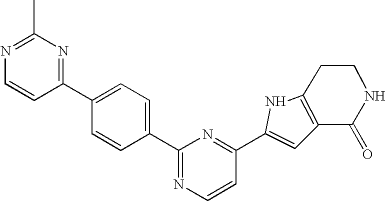 | 2-{2-[4-(2-methylpyrimidin-4-yl)phenyl]pyrimidin-4-yl}- 1,5,6,7-tetrahydro-4H-pyrrolo[3,2-c]pyridin-4-one |
| 1434 | 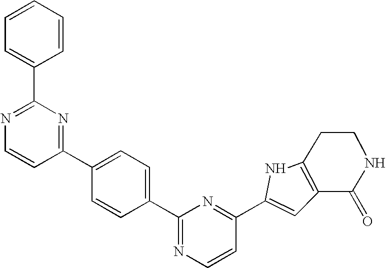 | 2-{2-[4-(2-phenylpyrimidin-4-yl)phenyl]pyrimidin-4-yl}- 1,5,6,7-tetrahydro-4H-pyrrolo[3,2-c]pyridin-4-one |
| 1435 | 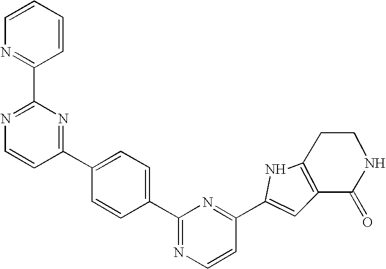 | 2-{2-[4-(2-pyridin-2-ylpyrimidin-4-yl)phenyl]pyrimidin-4-yl}- 1,5,6,7-tetrahydro-4H-pyrrolo[3,2-c]pyridin-4-one |
| 1436 | 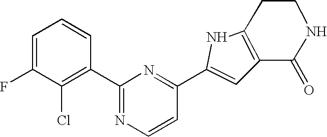 | 2-[2-(2-chloro-3-fluorophenyl)pyrimidin-4-yl]-1,5,6,7- tetrahydro-4H-pyrroIo[3,2-c]pyridin-4-one |
| 1437 | 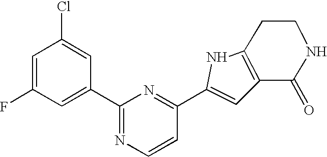 | 2-[2-(3-chloro-5-fluorophenyl)pyrimidin-4-yl]-1,5,6,7- tetrahydro-4H-prrolo[3,2-c]pyridin-4-one |
| 1438 | 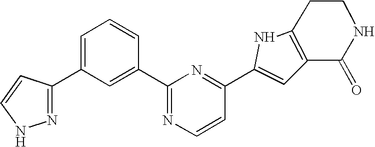 | 2-{2-[3-(1H-pyrazol-3-yl)phenyl]pyrimidin-4-yl]-1,5,6,7- tetrahydro-4H-pyrrolo[3,2-c]pyridin-4-one |
| 1439 | 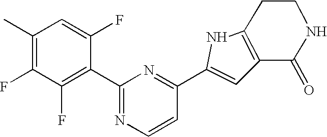 | 2-[2-(2,3,6-trifluoro-4-methylphenyl)pyrimidin-4-yl]-1,5,6,7- tetrahydro-4H-pyrrolo[3,2-c]pyridin-4-one |
| 1440 | 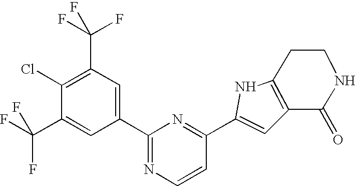 | 2-{2-[4-chloro-3,5-bis(trifluoromethyl)phenyl]pyrimidin-4- yl}-1,5,6,7-tetrahydro-4H-pyrrolo[3,2-c]pyridin-4-one |
| 1441 | 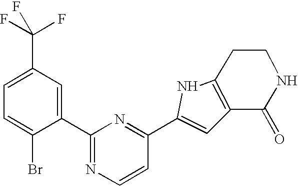 | 2-{2-[2-bromo-5-(trifluoromethyl)phenyl]pyrimidin-4-yl}- 1,5,6,7-tetrahydro-4H-pyrrolo[3,2-c]pyridin-4-one |
| 1442 | 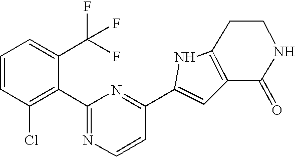 | 2-{2-[2-chloro-6-(trifluoromethyl)phenyl]pyrimidin-4-yl}- 1,5,6,7-tetrahydro-4H-pyrrolo[3,2-c]pyridin-4-one |
| 1443 | 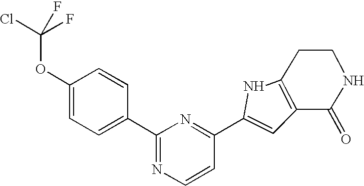 | 2-(2-{4-[chloro(difluoro)methoxy]phenyl}pyrimidin-4-yl)- 1,5,6,7-tetrahydro-4H-pyrrolo[3,2-c]pyridin-4-one |
| 1444 | 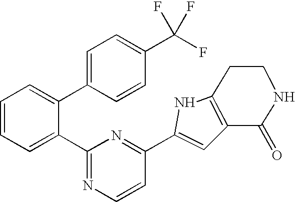 | 2-{2-[4-(trifluoromethyl)-1,1′-biphenyl-2-yl]pyrimidin-4-yl}- 1,5,6,7-tetrahydro-4H-pyrrolo[3,2-c]pyridin-4-one |
| 1445 | 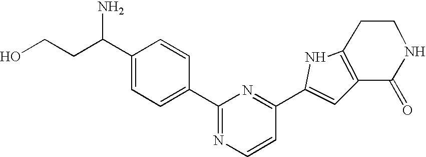 | 2-{2-[4-(1-amino-3-hydroxypropyl)phenyl]pyrimidin-4-yl}- 1,5,6,7-tetrahydro-4H-pyrrolo[3,2-c]pyridin-4-one |
| 1446 | 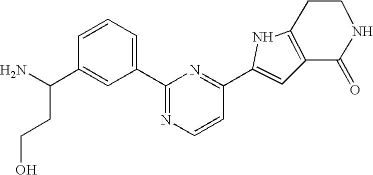 | 2-{2-[3-(1-amino-3-hydroxypropyl)phenyl]pyrimidin-4-yl}- 1,5,6,7-tetrahydro-4H-pyrrolo[3,2-c]pyridin-4-one |
| 1447 | 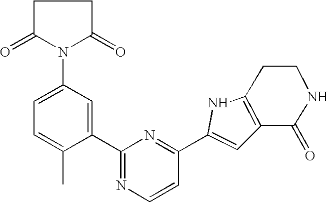 | 1-{4-methyl-3-[4-(4-oxo-4,5,6,7-tetrahydro-1H-pyrrolo[3,2- c]pyridin-2-yl)pyrimidin-2-yl]phenyl}pyrrolidine-2,5-dione |
| 1448 | 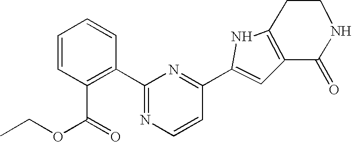 | ethyl 2-[4-(4-oxo-4,5,6,7-tetrahydro-1H-pyrrolo[3,2- c]pyridin-2-yl)pyrimidin-2-yl]benzoate |
| 1449 | 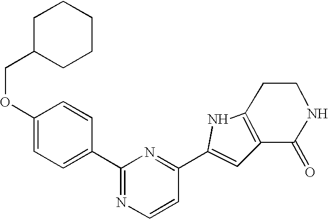 | 2-{2-[4-(cyclohexylmethoxy)phenyl]pyrimidin-4-yl}-1,5,6,7- tetrahydro-4H-pyrrolo[3,2-c]pyridin-4-one |
| 1450 | 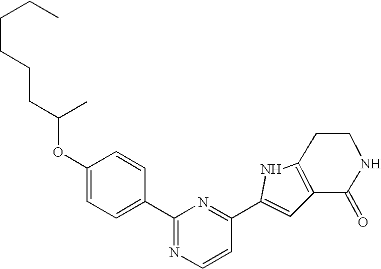 | 2-(2-{4-[(1-methylheptyl)oxy]phenyl}pyrimidin-4-yl)-1,5,6,7- tetrahydro-4H-pyrrolo[3,2-c]pyridin-4-one |
| 1451 | 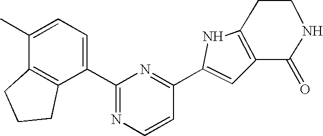 | 2-[2-(7-methyl-2,3-dihydro-1H-inden-4-yl)pyrimidin-4-yl]- 1,5,6,7-tetrahydro-4H-pyrrolo[3,2-c]pyridin-4-one |
| 1452 | 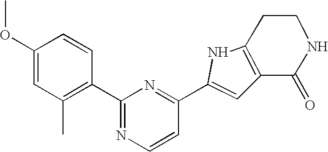 | 2-[2-(4-methoxy-2-methylphenyl)pyrimidin-4-yl]-1,5,6,7- tetrahydro-4H-pyrrolo[3,2-c]pyridin-4-one |
| 1453 | 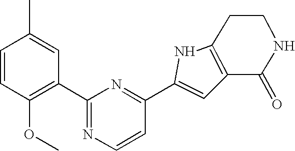 | 2-[2-(2-methoxy-5-methylphenyl)pyrimidin-4-yl]-1,5,6,7- tetrahydro-4H-pyrrolo[3,2-c]pyridin-4-one |
| 1454 | 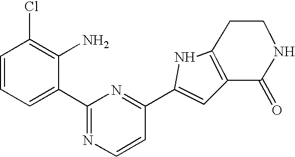 | 2-[2-(2-amino-3-chlorophenyl)pyrimidin-4-yl]-1,5,6,7- tetrahydro-4H-pyrrolo[3,2-c]pyridin-4-one |
| 1455 | 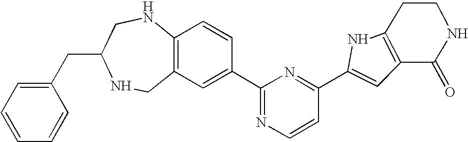 | 2-[2-(3-benzyl-2,3,4,5-tetrahydro-1H-1,4-benzodiazepin-7- yl)pyrimidin-4-yl]-1,5,6,7-tetrahydro-4H-pyrrolo[3,2- c]pyridin-4-one |
| 1456 | 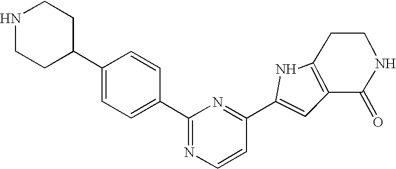 | 2-[2-(4-piperidin-4-ylphenyl)pyrimidin-4-yl]-1,5,6,7- tetrahydro-4H-pyrrolo[3,2-c]pyridin-4-one |
| 1457 | 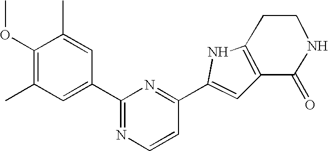 | 2-[2-(4-methoxy-3,5-dimethylphenyl)pyrimidin-4-yl]- 1,5,6,7-tetrahydro-4H-pyrrolo[3,2-c]pyridin-4-one |
| 1458 | 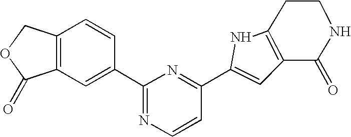 | 2-[2-(3-oxo-1,3-dihydro-2-benzofuran-5-yl)pyrimidin-4-yl]- 1,5,6,7-tetrahydro-4H-pyrrolo[3,2-c]pyridin-4-one |
| 1459 | 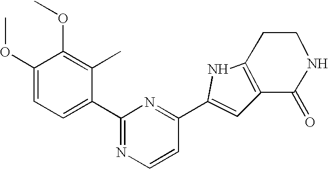 | 2-[2-(3,4-dimethoxy-2-methylphenyl)pyrimidin-4-yl]- 1,5,6,7-tetrahydro-4H-pyrrolo[3,2-c]pyridin-4-one |
| 1460 | 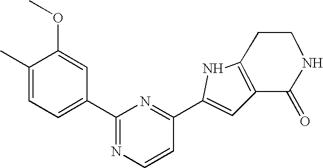 | 2-[2-(3-methoxy-4-methylphenyl)pyrimidin-4-yl]-1,5,6,7- tetrahydro-4H-pyrrolo[3,2-c]pyridin-4-one |
| 1461 | 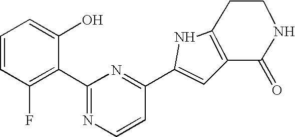 | 2-[2-(2-fluoro-6-hydroxyphenyl)pyrimidin-4-yl]-1,5,6,7- tetrahydro-4H-pyrrolo[3,2-c]pyridin-4-one |
| 1462 | 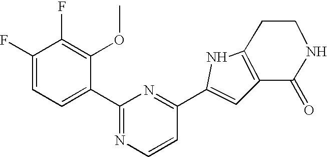 | 2-[2-(3,4-difluoro-2-methoxyphenyl)pyrimidin-4-yl]-1,5,6,7- tetrahydro-4H-pyrrolo[3,2-c]pyridin-4-one |
| 1463 | 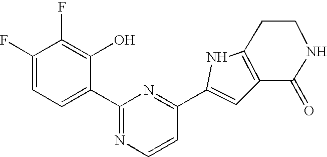 | 2-[2-(3,4-difluoro-2-hydroxyphenyl)pyrimidin-4-yl]-1,5,6,7- tetrahydro-4H-pyrrolo[3,2-c]pyridin-4-one |
| 1464 | 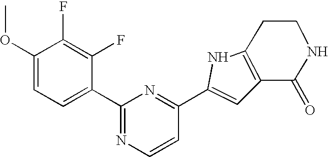 | 2-[2-(2,3-difluoro-4-methoxyphenyl)pyrimidin-4-yl]-1,5,6,7- tetrahydro-4H-pyrrolo[3,2-c]pyridin-4-one |
| 1465 | 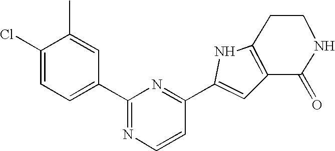 | 2-[2-(4-chloro-3-methylphenyl)pyrimidin-4-yl]-1,5,6,7- tetrahydro-4H-pyrrolo[3,2-c]pyridin-4-one |
| 1466 | 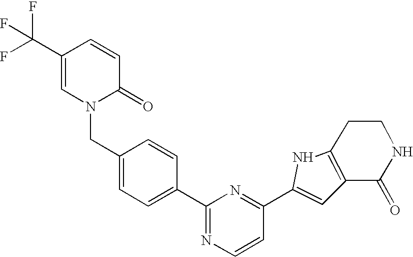 | 2-[2-(4-{[2-oxo-5-(trifluoromethyl)pyridin-1(2H)- yl]methyl}phenyl)pyrimidin-4-yl]-1,5,6,7-tetrahydro-4H- pyrrolo[3,2-c]pyridin-4-one |
| 1467 | 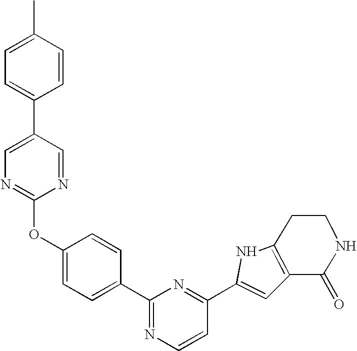 | 2-[2-(4-{[5-(4-methylphenyl)pyrimidin-2- yl]oxy}phenyl)pyrimidin-4-yl]-1,5,6,7-tetrahydro-4H- pyrrolo[3,2-c]pyridin-4-one |
| 1468 | 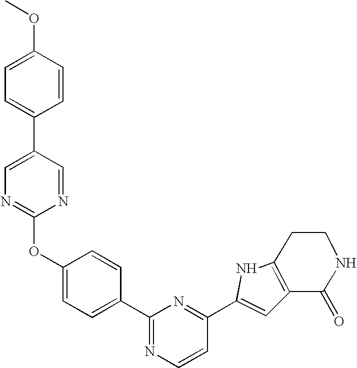 | 2-[2-(4-{[5-(4-methoxyphenyl)pyrimidin-2- yl]oxy}phenyl)pyrimidin-4-yl]-1,5,6,7-tetrahydro-4H- pyrrolo[3,2-c]pyridin-4-one |
| 1469 | 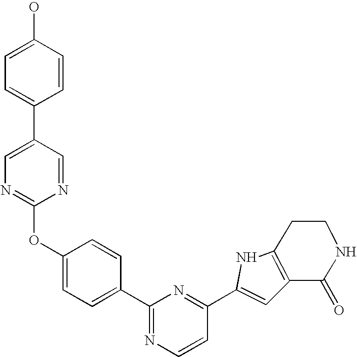 | 2-[2-(4-{[5-(4-fluorophenyl)pyrimidin-2- yl]oxy}phenyl)pyrimidin-4-yl]-1,5,6,7-tetrahydro-4H- pyrrolo[3,2-c]pyridin-4-one |
| 1470 | 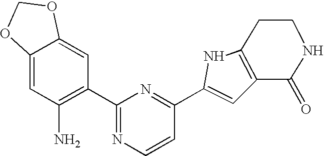 | 2-[2-(6-amino-1,3-benzodioxol-5-yl)pyrimidin-4-yl]-1,5,6,7- tetrahydro-4H-pyrrolo[3,2-c]pyridin-4-one |
| 1471 | 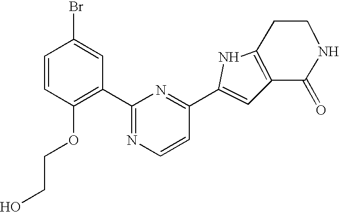 | 2-{2-[5-bromo-2-(2-hydroxyethoxy)phenyl]pyrimidin-4-yl]- 1,5,6,7-tetrahydro-4H-pyrrolo[3,2-c]pyridin-4-one |
| 1472 | 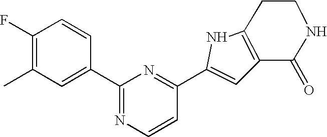 | 2-[2-(4-fluoro-3-methylphenyl)pyrimidin-4-yl]-1 5,6,7- tetrahydro-4H-pyrrolo[3,2-c]pyridin-4-one |
| 1473 | 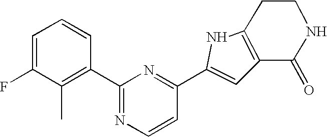 | 2-[2-(3-fluoro-2-methylphenyl)pyrimidin-4-yl]-1,5,6,7- tetrahydro-4H-pyrrolo[3,2-c]pyridin-4-one |
| 1474 | 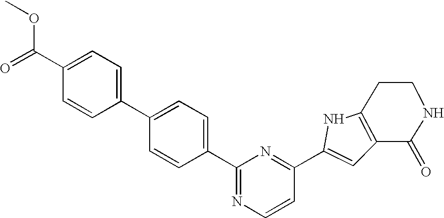 | methyl 4′-[4-(4-oxo-4,5,6,7-tetrahydro-1H-pyrrolo[3,2- c]pyridin-2-yl)pyrimidin-2-yl]-1,1′-biphenyl-4-carboxylate |
| 1475 | 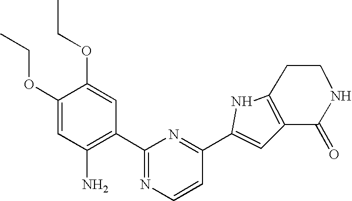 | 2-[2-(2-amino-4,5-diethoxyphenyl)pyrimidin-4-yl]-1,5,6,7- tetrahydro-4H-pyrrolo[3,2-c]pyridin-4-one |
| 1476 | 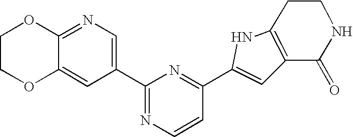 | 2-[2-(2,3-dihydro[1,4]dioxino[2,3-b]pyridin-7-yl)pyrimidin-4- yl]-1,5,6,7-tetrahydro-4H-pyrrolo[3,2-c]pyridin-4-one |
| 1477 | 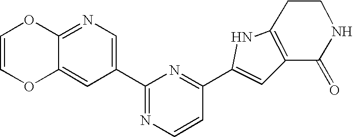 | 2-(2-[1,4]dioxino[2,3-b]pyridin-7-ylpyrimidin-4-yl)-1,5,6,7- tetrahydro-4H-pyrrolo[3,2-c]pyridin-4-one |
| 1478 | 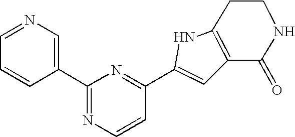 | 2-(2-pyridin-3-ylpyrimidin-4-yl)-1,5,6,7-tetrahydro-4H- pyrrolo[3,2-c]pyridin-4-one |
| 1479 | 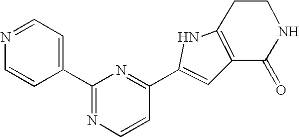 | 2-(2-pyridin-4-ylpyrimidin-4-yl)-1,5,6,7-tetrahydro-4H- pyrrolo[3,2-c]pyridin-4-one |
| 1480 | 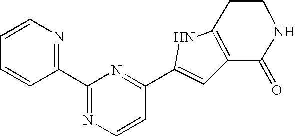 | 2-(2-pyridin-2-ylpyrimidin-4-yl)-1,5,6,7-tetrahydro-4H- pyrrolo[3,2-c]pyridin-4-one |
| 1481 | 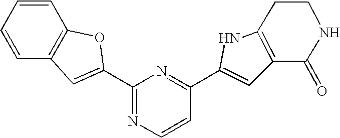 | 2-[2-(1-benzofuran-2-yl)pyrimidin-4-yl]-1,5,6,7-tetrahydro- 4H-pyrrolo[3,2-c]pyridin-4-one |
| 1482 | 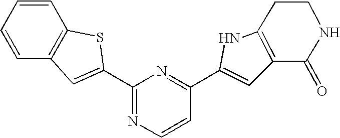 | 2-[2-(1-benzothien-2-yl)pyrimidin-4-yl]-1,5,6,7-tetrahydro- 4H-pyrrolo[3,2-c]pyridin-4-one |
| 1483 | 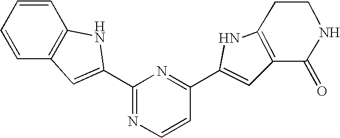 | 2-[2-(1H-indol-2-yl)pyrimidin-4-yl]-1,5,6,7-tetrahydro-4H- pyrrolo[3,2-c]pyridin-4-one |
| 1484 | 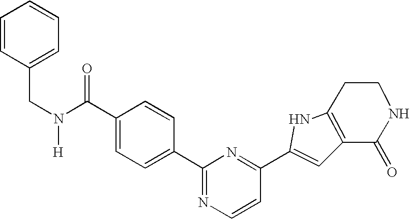 | N-benzyl-4-[4-(4-oxo-4,5,6,7-tetrahydro-1H-pyrrolo[3,2- c]pyridin-2-yl)pyrimidin-2-yl]benzamide |
| 1485 | 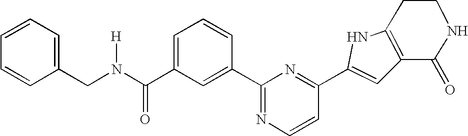 | N-benzyl-3-[4-(4-oxo-4,5,6,7-tetrahydro-1H-pyrrolo[3,2- c]pyridin-2-yl)pyrimidin-2-yl]benzamide |

Notes:
^{a}Chemical names were generated by ACD/Name software.
^{b}The MK-2 inhibiting compound may be shown with a solvent, such as, for example, trifluoroacetate, with which it can form a salt. Both the salt and base forms of the pyrrole compound are included in the present invention.

In one embodiment of the present invention, the MK-2 inhibiting compound is one that is listed in Table I or in Table II. It is preferred that the MK-2 inhibiting compound is one that has an IC₅₀ value for the inhibition of MK-2 that is lower than 1. By way of example, this would include the compounds in Table I numbered 1-681. An MK-2 IC₅₀ value that is lower than 0.5 is more preferred (examples of these compounds include the compounds in Table I numbered 1-633), lower than 0.1 is even more preferred (examples of these compound include the compounds in Table I numbered 1-432), lower than 0.05 is yet more preferred (examples of these compound include the compounds in Table I numbered 1-273), and lower than 0.01 is even more preferred (examples of these compound include the compounds in Table I numbered 1-25).

In another embodiment, the present MK-2 inhibiting compound has the structure shown in formula III: Formula III:

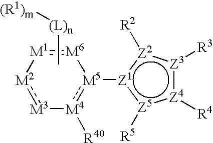

wherein

dashed lines indicate optional single or double bonds;

Z¹, Z², Z³, Z⁴, Z⁵, M¹ and M⁵ are independently selected from nitrogen or carbon;

Z¹, Z², Z³, Z⁴ and Z⁵ join to form a ring that is selected from pyrrole, isopyrrole, triazole, imidazole, and tetrazole;

M², M³, M⁴ and M⁶ are independently selected from carbon, nitrogen, oxygen, and sulfur;

L is selected from carboxyamino, carboxyaminoalkyl, alkenyl, alkynyl, alkyl, hydrazoalkyl, arylcarbamyl, aryl, heteroaryl, arylalkyl, arylalkylamino, and alkylaryl,

n is an integer that is selected from 0, or 1;

R¹ is optionally absent, or each R¹ is independently selected from cycloalkyl, aryl, , heteroaryl, halo, heterocyclyl, cyano, alkyl, alkenyl, alkynyl, alkoxy, amino, hydroxy, carboalkoxy, alkylthio, haloalkyl, carboxyl, haloalkoxy, acetyl, alkoxyaryl, hydroxyalkyl, carbamyl, cycloalkylalkyl, carboxyalkyl, alkylamino, carboxyalkenyl, nitro, cyanoalkyl, and arylalkoxy, where aryl, heteroaryl and heterocyclyl can be substituted or unsubstituted;

m is an integer selected from 0, 1, 2, 3, 4, or 5;

R², R³, R⁴ and R⁵ are optionally absent, or each of R², R³, R⁴ and R⁵ is independently selected from hydrogen, alkyl, carboxyaminoalkyl, carboxyl, heterocyclyl, aminoalkyl, carbamylamino, carboxyalkyl, haloalkyl, aryl, or R³ and R⁴ optionally join to form a ring having the structure:

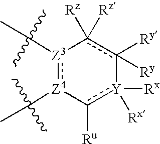

; where

dashed lines indicate optional single or double bonds;

Y is selected from carbon or nitrogen;

R^{u}, R^{x}, R^{x}′, R^{y}, R^{y}′^{,} R^{z}, and R^{z}′ are optionally absent, or are independently selected from hydrogen, oxo, hydroxy, and carboxyalkyl; and

R⁴⁰ is optionally absent, or is hydrogen, or

R⁴⁰ and R⁵ optionally join to form a six-membered ring.

Another embodiment of the present MK-2 inhibiting compounds comprises a compound having the structure shown in formula III, where:

L is selected from carboxyamino, carboxyamino-C₁-C₄-alkyl, C₁-C₆-alkenyl, C₁-C₆-alkynyl, C₁-C₆-alkyl, hydrazo-C₁-C₄-alkyl, arylcarbamyl, aryl, heteroaryl, aryl-C₁-C₄-alkyl, aryl-C₁-C₄-alkylamino, and C₁-C₄-alkylaryl;

n is an integer that is selected from 0, and 1;

R¹ is optionally absent, or each R¹ is independently selected from cyclo-C₁-C₄-alkyl, aryl, heteroaryl, halo, heterocyclyl, cyano, C₁-C₆-alkyl, C₁-C₆-alkenyl, C₁-C₆-alkynyl, C₁-C₄-alkoxy, amino, hydroxy, carboxy-C₁-C₄-alkoxy, C₁-C₄-alkylthio, halo-C₁-C₄-alkyl, carboxyl, halo-C₁-C₄-alkoxy, acetyl, C₁-C₄-alkoxyaryl, hydroxy-C₁-C₄-alkyl, carbamyl, cyclo-C₁-C₄-alkyl-C₁-C₄-alkyl, carboxy-C₁-C₄-alkyl, C₁-C₄-alkylamino, carboxy-C₁-C₄-alkenyl, nitro, cyano-C₁-C₄-alkyl, and aryl-C₁-C₄-alkoxy, where aryl, heteroaryl and heterocyclyl can be substituted or unsubstituted;

m is an integer selected from 0, 1, 2, 3, 4, or 5;

R², R³, R⁴ and R⁵ are optionally absent, or each of R², R³, R⁴ and R⁵ is independently selected from hydrogen, C₁-C₆-alkyl, carboxyamino-C₁-C₄-alkyl, carboxyl, heterocyclyl, amino-C₁-C₄-alkyl, carbamylamino, carboxy-C₁-C₄-alkyl, halo-C₁-C₄-alkyl, aryl, or R³ and R⁴ optionally join to form a ring having the structure:

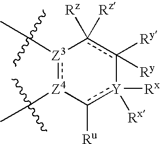

; where

dashed lines indicate optional single or double bonds;

Y is selected from carbon or nitrogen;

R^{u}, R^{x}, R^{x}′, R^{y}, R^{y}′, R^{z}, and R^{z}′ are optionally absent, or are independently selected from hydrogen, oxo, hydroxy, and carboxy-C₁-C₄-alkyl; and

R⁴⁰ is optionally absent, or is hydrogen, or

R⁴⁰ and R⁵ optionally join to form a six-membered ring.

Another embodiment of the present MK-2 inhibiting compounds comprises a compound having the structure shown in formula III, where:

L is selected from —CONH—, —CON(CH₃)——(CH)═(CH)—, —(CH)═C(CH₃)—, —CONH—(CH₂)—, —NH—NH═CH—, —(C₆H₄)—CONH—, —(C₆H₄)—, pyridyl, styryl, —(CH)═(CH)—(CH)═(CH)—, —(C₆H₄)—(CH)₂—NH—, —(CH₂)—, —(C₆H₃F)—CONH—, and —(CH₂)—(CH₂)-(phenyl)-;

n is an integer that is selected from 0, or 1;

R¹ is optionally absent, or each R¹ is independently selected from cyclopentyl, phenyl, quinolyl, hydroxynaphthyl, fluoro, indolyl, cyano, benzodioxol, butyl, cyclopropyl, methoxyl, cyclohexyl, pyridyl, ethyl, amino, thienyl, hydroxy, carbomethoxy, methylthio, trifluoromethyl, carboxyl, methyl, dihydroisoquinolyl, chloro, trifluoromethoxy, acetyl, ethoxy, methoxynaphthyl, hydroxymethyl, hydroxyethyl, carbamyl, cyclopropylmethyl, carboxyethyl, imidazoyl, benzothienyl, pyrimidyl, hydroxypropyl, butoxy, dimethylamino, furyl, imidazoyl, carboxyethenyl, isopropyl, nitro, propyl, piperidylcarbonyl, cyanomethyl, phenylmethoxyl, styryl, and —COO-(tert-butyl)indoyl;

m is an integer selected from 0, 1, 2, 3, 4, or 5;

R², R³, R⁴ and R⁵ are optionally absent, or each of R², R³, R⁴ and R⁵ is independently selected from hydrogen, methyl, —CO—N(CH₃)₂, carboxyl, pyridyl, aminoethyl, —CO—NH—NH₂, —COO-(tert-butyl), trifluoromethane, benzyl, or R³ and R⁴ optionally join to form a ring having the structure:

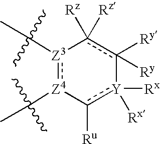

where;

dashed lines indicate optional single or double bonds;

Y is selected from carbon or nitrogen;

R^{u}, R^{x}, R^{x}′, R^{y}, R^{y}′, R^{z}, and R^{z}′ are optionally absent, or are independently selected from hydrogen, oxo, hydroxy, and —COO-(tert-butyl); and

R⁴⁰ is optionally absent, or is hydrogen, or R⁴⁰ and R⁵ optionally join to form a six-membered ring.

Another embodiment of the present MK-2 inhibiting compounds comprises a compound having the structure shown in formula III, where:

L is selected from —CONH—, —CON(CH₃)——(CH)═(CH)—, —(CH)═C(CH₃)—, —NH—NH═CH—, —(C₆H₄)—CONH—, —(C₆H₄)—, pyridyl, styryl, —(CH)═(CH)—(CH)═(CH)—, —(C₆H₄)—(CH)₂—NH—, and —(C₆H₃F)—CONH—;

n is an integer that is selected from 0, or 1;

R¹ is optionally absent, or each R¹ is independently selected from cyclopentyl, phenyl, quinolyl, hydroxynaphthyl, fluoro, indolyl, cyano, benzodioxol, butyl, cyclopropyl, methoxyl, cyclohexyl, pyridyl, ethyl, amino, thienyl, hydroxy, carbomethoxy, methylthio, trifluoromethyl, carboxyl, methyl, dihydroisoquinolyl, chloro, trifluoromethoxy, acetyl, ethoxy, methoxynaphthyl, hydroxymethyl, hydroxyethyl, carbamyl, cyclopropylmethyl, carboxyethyl, imidazoyl, benzothienyl, pyrimidyl, hydroxypropyl, butoxy, dimethylamino, furyl, imidazoyl, carboxyethenyl, isopropyl, nitro, propyl, piperidylcarbonyl, cyanomethyl, phenylmethoxyl, styryl, and —COO-(tert-butyl)indoyl;

m is an integer selected from 0, 1, 2, 3, 4, or 5;

R² and R⁵ are optionally absent, or each of R² and R⁵ is independently selected from hydrogen, methyl, —CO—N(CH₃)₂, carboxyl, pyridyl, aminoethyl, —CO—NH—NH₂, —COO-(tert-butyl), trifluoromethane, and benzyl;

R³ and R⁴ join to form a ring having the structure:

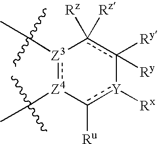

; where

dashed lines indicate optional single or double bonds;

Y is nitrogen;

R^{u}, R^{x}, R^{y}, R^{y}′, R^{z}, and R^{z}′ are optionally absent, or are independently selected from hydrogen, and oxo; and

R⁴⁰ is optionally absent, or is hydrogen, or R⁴⁰ and R⁵ optionally join to form a six-membered ring.

Another embodiment of the present MK-2 inhibiting compounds comprises a compound having the structure shown in formula III, where:

L is selected from —(CH)═(CH)—, —NH—NH═CH—, —(C₆H₄)—CONH—, —(C₆H₄)—, pyridyl, styryl, —(CH)═(CH)—(CH)═(CH)—, —(C₆H₄)—(CH)₂—NH—, and —(C₆H₃F)—CONH—;

n is an integer that is selected from 0, or 1;

R¹ is optionally absent, or each R¹ is independently selected from cyclopentyl, phenyl, quinolyl, hydroxynaphthyl, fluoro, indolyl, cyano, benzodioxol, butyl, cyclopropyl, methoxyl, cyclohexyl, pyridyl, ethyl, amino, thienyl, hydroxy, carbomethoxy, methylthio, trifluoromethyl, carboxyl, methyl, dihydroisoquinolyl, chloro, trifluoromethoxy, acetyl, ethoxy, methoxynaphthyl, hydroxymethyl, hydroxyethyl, carbamyl, cyclopropylmethyl, carboxyethyl, imidazoyl, benzothienyl, pyrimidyl, hydroxypropyl, and styryl;

m is an integer selected from 0, 1, 2, 3, 4, or 5;

R² and R⁵ are optionally absent, or each of R² and R⁵ is independently selected from hydrogen, methyl, —CO—N(CH₃)₂, carboxyl, pyridyl, aminoethyl, —CO—NH—NH₂, —COO-(tert-butyl), trifluoromethane, and benzyl;

R³ and R⁴ join to form a ring having the structure:

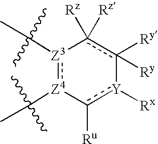

; where

dashed lines indicate optional single or double bonds;

Y is nitrogen;

R^{u}, R^{x}, R^{y}, R^{y}′, R^{z}, and R^{z}′ are optionally absent, or are independently selected from hydrogen, and oxo; and

R⁴⁰ is optionally absent, or is hydrogen, or R⁴⁰ and R⁵ optionally join to form a six-membered ring.

Another embodiment of the present MK-2 inhibiting compounds comprises a compound having the structure shown in formula III, where:

L is selected from —(CH)═(CH)—, —NH—NH═CH—, —(C₆H₄)—CONH—, —(C₆H₄)—, pyridyl, and styryl;

n is an integer that is selected from 0, or 1;

R¹ is optionally absent, or each R¹ is independently selected from cyclopentyl, phenyl, quinolyl, hydroxynaphthyl, fluoro, indolyl, cyano, benzodioxol, butyl, cyclopropyl, methoxyl, cyclohexyl, pyridyl, ethyl, amino, and styryl;

m is an integer selected from 0, 1, 2, 3, 4, or 5;

R² and R⁵ are optionally absent, or each of R² and R⁵ is independently selected from hydrogen, methyl, —CO—N(CH₃)₂, carboxyl, pyridyl, aminoethyl, —CO—NH—NH₂, —COO-(tert-butyl), trifluoromethane, and benzyl;

R³ and R⁴ join to form a ring having the structure:

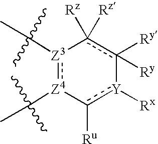

; where

dashed lines indicate optional single or double bonds;

Y is nitrogen;

R^{u}, R^{x}, R^{y}, R^{y}′, R^{z}, and R^{z}′ are optionally absent, or are independently selected from hydrogen, and oxo; and

R⁴⁰ is optionally absent, or is hydrogen, or R⁴⁰ and R⁵ optionally join to form a six-membered ring.

Another embodiment of the present MK-2 inhibiting compounds comprises a compound having the structure shown in formula III, where:

M¹, M³, M⁴, M⁵ and M⁶ are carbon;

M² is nitrogen;

L is selected from —NH—NH═CH—, —(C₆H₄)—CONH—, and styryl;

n is an integer that is selected from 0, or 1;

R¹ is optionally absent, or each R¹ is independently selected from cyclopentyl, phenyl, quinolyl, hydroxynaphthyl, fluoro, and styryl;

m is an integer selected from 0, 1, 2, 3, 4, or 5;

R² and R⁵ are optionally absent, or each of R² and R⁵ is hydrogen;

R³ and R⁴ join to form a ring having the structure:

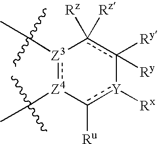

; where

dashed lines indicate optional single or double bonds;

Y is nitrogen;

R^{u}, R^{x}, R^{y}, R^{y}′, R^{z}, and R^{z}′ are optionally absent, or are independently selected from hydrogen, and oxo; and

R⁴⁰ is hydrogen.

Another embodiment of the present MK-2 inhibiting compounds comprises a compound having the structure shown in formula IV: Formula IV:

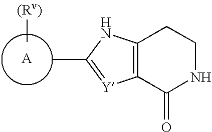

where:

Y′ is selected from CR⁴¹ or nitrogen;

A is a substituted or unsubstituted heterocyclic, heteroaryl, or aryl ring;

when A is substituted, it can have from 1 to 6 R^{v} substituent groups;

R^{v} is optionally absent, or each R^{v} is selected from hydrogen, halo or an organic radical; and

R⁴¹ is selected from hydrogen, halo, or an organic radical, or

R⁴¹ optionally joins with any R^{v} to form a ring structure.

Another embodiment of the present MK-2 inhibiting compounds comprises a compound having the structure shown in formula V: Formula V:

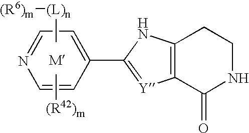

where:

R⁵ is selected from —H, C₁-C₆ alkyl, C₂-C₆ alkenyl, C₂-C₆ alkynyl, C₁-C₆ alkyl-R¹¹, C₂-C₆ alkenyl-R¹¹, C₂-C₆ alkynyl-R¹¹, C₁-C₆ alkyl-(R¹¹)₂, C₂-C₆ alkenyl-(R¹¹)₂, CSR¹¹, N═NR⁷, amino, NHR⁷, NR⁸R⁹, N(R⁷)—N(R⁸)(R⁹), ═N—N(R⁸)(R⁹), N═N(R⁷), N(R⁷)—N═(R⁸), C₁-C₆ alkyl-NHR⁷, C₁-C₆ alkyl-NR⁸R⁹, (C₁-C₄)alkyl-N(R⁷)—N(R⁸)(R⁹), (C₁-C₄)alkyl═N—N(R⁸)(R⁹), (C₁-C₄)alkyl-N═N(R⁷), (C₁-C₄)alkyl-N(R⁷)—N═(R⁸), nitro, cyano, O—R¹⁰, C₁-C₄ alkyl-OR¹⁰, COR¹¹, SR¹⁰, SOR¹¹, SO₂R^{11, C}₁-C₆ alkyl-COR¹¹, C₁-C₆ alkyl-SR¹⁰, C₁-C₆ alkyl-SOR¹¹, C₁-C₆ alkyl-SO₂R¹¹, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹²;

R⁷, R⁸, are each independently selected from —H, C₁-C₆ alkyl, C₂-C₆ alkenyl, C₂-C₆ alkynyl, C₁-C₄ alkyl-R¹¹, amino, NHR¹³, NR¹³R¹⁴, C₁-C₆ alkyl-NHR¹³, C₁-C₆ alkyl-NR¹³R¹⁴, O—R¹⁵, C₁-C₄ alkyl-OR¹⁵, CO₂R¹⁶, COR¹⁷, CO(R¹⁷)₂, CONHR¹⁶, CON(R¹⁶)₂, SR¹⁵, SOR¹⁷, SO₂R¹⁷, C₁-C₆ alkyl-CO₂R¹⁶, C₁-C₆ alkyl-COR¹⁷, C₁-C₆ alkyl-CO₂R¹⁷, C₁-C₆ alkyl-CONHR¹⁶, C₁-C₆ alkyl-CON(R¹⁶)₂, C₁-C₆ alkyl-SR¹⁵, C₁-C₆ alkyl-SOR¹⁷, C₁-C₆ alkyl-SO₂R¹⁷, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R⁹, R¹⁰ are each independently selected from —H, C₁-C₆ alkyl, C₂-C₆ alkenyl, C₂-C₆ alkynyl, C₁-C₆ alkyl-NHR¹³, C₁-C₆ alkyl-NR¹³R¹⁴, C₁-C₄ alkyl-OR¹⁵, CSR¹¹, CO₂R¹⁶, COR¹⁷, CONHR¹⁶, CON(R¹⁶)₂, SOR¹⁷, SO₂R¹⁷, C₁-C₆ alkyl-CO₂R¹⁷, C₁-C₆ alkyl-COR¹⁷, C₁-C₆ alkyl-CONHR¹⁶, C₁-C₆ alkyl-CON(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹¹ is selected from —H, C₁-C₆ alkyl, C₁-C₆ alkoxy, C₂-C₆ alkenyl, C₂-C₆ alkynyl, amino, NHR¹³, NR¹³R¹⁴, N═NR¹³, C₁-C₆ alkyl-NHR , C₁-C₆ ¹¹alkyl-NR¹³R¹⁴, O—R¹⁵, C₁-C₄ alkyl-OR¹⁵, SR¹⁵, C₁-C₆ alkyl-CO₂R¹⁶, C₁-C₆ alkyl-COR¹⁷, C₁-C₆ alkyl-CONHR¹⁶, C₁-C₆ alkyl-CON(R¹⁶)₂, C₁-C₆ alkyl-SR¹⁵, C₁-C₆ alkyl-SOR¹⁷, C₁-C₆ alkyl-SO₂R¹⁷, halo, halo C₁-C₄ alkyl heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹² is selected from —H, OH, C₁-C₆ alkyl, C₂-C₆ alkenyl, C₂-C₆ alkynyl, amino, NHR⁷, NR⁸R⁹, C₁-C₆ alkyl-NHR⁷, C₁-C₆ alkyl-NR⁸R⁹, nitro, cyano, O—R¹⁰, C₁-C₄ alkyl-OR¹⁰, COR¹¹, CO₂R¹¹, SR¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, halo C₁-C₄ alkyl, hydroxy C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;

R¹³ and R¹⁴ are each independently selected from —H, C₁-C₆ alkyl, C₂-C₆ alkenyl, C₂-C₆ alkynyl, amino, C₁-C₆ alkyl-NHR¹⁹, C₁-C₆ alkyl-NR¹⁹R²⁰, C₁-C₄ alkyl-OR²¹, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁵, R¹⁶ are each independently selected from —H, C₁-C₆ alkyl, C₂-C₆ alkenyl, C₂-C₆ alkynyl, C₁-C₆ alkyl-NHR¹⁹, C₁-C₆ alkyl-NR¹⁹R²⁰, C₁-C₄ alkyl-OR²¹, CO₂R²², COR²³, CONHR²², CON(R²²)₂, SOR²³, SO₂R²⁴, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁷ is selected from —H, C₁-C₆ alkyl, C₂-C₆ alkenyl, C₂-C₆ alkenyl-R¹⁹, C₁-C₆ alkyl-R¹⁹, C₂-C₆ alkynyl, amino, NHR¹⁹, NR¹⁹R²⁰, C₁-C₆ alkyl-NHR¹⁹, C₁-C₆ alkyl-NR¹⁹R²⁰, O—R²¹, C₁-C₄ alkyl-OR²¹, SR²¹, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁸ is selected from —H, C₁-C₆ alkyl, C₂-C₆ alkenyl, C₂-C₆ alkynyl, amino, NHR¹⁹, NR¹⁹R²⁰, C₁-C₆ alkyl-NHR¹⁹, C₁-C₆ alkyl-NR¹⁹R²⁰, nitro, cyano, O—R²¹, C₁-C₄ alkyl-OR²¹, aryl, heteroaryl, heterocyclyl, COR²³, SR²¹, SOR²³, SO₂R²³, C₁-C₆ alkyl-COR²³, C₁-C₆ alkyl-SR²¹, C₁-C₆ alkyl-SOR²³, C₁-C₆ alkyl-SO₂R²³, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;

R¹⁹ and R²⁰ are each independently selected from —H, C₁-C₆ alkyl, C₂-C₆ alkenyl, C₂-C₆ alkynyl, amino, C₁-C₆ alkyl-NHR²⁵, C₁-C₆ alkyl-NR²⁵R²⁶, C₁-C₄ alkyl-OR²⁷, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²¹ and R²² are each independently selected from —H, C₁-C₆ alkyl, C₂-C₆ alkenyl, C₂-C₆ alkynyl, C₁-C₆ alkyl-NHR²⁵, C₁-C₆ alkyl-NR²⁵R²⁶, C₁-C₄ alkyl-OR²⁷, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²³ is selected from —H, C₁-C₆ alkyl, C₂-C₆ alkenyl, C₂-C₆ alkynyl, amino, NHR²⁵, NR²⁵R²⁶, C₁-C₆ alkyl-NHR²⁵, C₁-C₆ alkyl-NR²⁵R²⁶, O—R²⁷, C₁-C₄ alkyl-OR²⁷, SR²⁷, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁴ is selected from —H, C₁-C₆ alkyl, C₂-C₆ alkenyl, C₂-C₆ alkynyl, amino, NHR²⁵, NR²⁵R²⁶, C₁-C₆ alkyl-NHR²⁵, C₁-C₆ alkyl-NR²⁵R²⁶, nitro, cyano, O—R²⁷, C₁-C₄ alkyl-OR²⁷, COR²⁹, SR²⁷, SOR²⁹, SO₂R²⁹, C₁-C₆ alkyl-COR²⁹, C₁-C₆ alkyl-SR²⁷, C₁-C₆ alkyl-SOR²⁹, C₁-C₆ alkyl-SO₂R²⁹, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;

R²⁵ and R²⁶ are each independently selected from —H, C₁-C₆ alkyl, C₂-C₆ alkenyl, C₂-C₆ alkynyl, amino, C₁-C₆ alkyl-NHR³¹, C₁-C₆ alkyl-NR³¹R³², C₁-C₄ alkyl-OR³³, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONH³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁷ and R²⁸ are each independently selected from —H, C₁-C₆ alkyl, C₂-C₆ alkenyl, C₂-C₆ alkynyl, C₁-C₆ alkyl-NHR³¹, C₁-C₆ alkyl-NR³¹R³², C₁-C₄ alkyl-OR³³, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R²⁹ is selected from —H, C₁-C₆ alkyl, C₂-C₆ alkenyl, C₂-C₆ alkynyl, amino, NHR³¹, NR³¹R³², C₁-C₆ alkyl-NHR³¹, C₁-C₆ alkyl-NR³¹R³², O—R³³, C₁-C₄ alkyl-OR³³, SR³³, C₁-C₆ alkyl-CO₂R³⁴, C₁-C₆ alkyl-COR³⁵, C₁-C₆ alkyl-CONHR³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁰ is selected from —H, C₁-C₆ alkyl, C₂-C₆ alkenyl, C₂-C₆ alkynyl, C₁-C₄ alkyl-R³¹, amino, NHR³¹, NR³¹R³², C₁-C₆ alkyl-NHR³¹, C₁-C₆ alkyl-NR³¹R³², nitro, cyano, O—R³³, C₁-C₄ alkyl-OR³³, COR³⁵, SR³³, SOR³⁵, SO₂R³⁵, C₁-C₆ alkyl-COR³⁵, C₁-C₆ alkyl-SR³³, C₁-C₆ alkyl-SOR³⁵, C₁-C₆ alkyl-SO₂R³⁵, halo, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³¹, R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;

R³⁶ is selected from alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, and heteroarylalkyl;

L is selected from C(R³⁷)₂, O, S, NR³⁷, C═O, C═S, C═C(R³⁷)₂, SO, SO₂, N═NO, CR³⁷═CR³⁷, CR³⁷═N, N═CR³⁷, N═N, NO═N, C═ONR³⁷, C═SR³⁷, NR³⁷C═O, NR³⁷C═S, C═OO, C═OS, C═SO, C═SS, OC═O, SC═O, OC═S, SC═S, S(O)ₘ—(O,S,NR³⁷), (O,S,NR³⁷—S(O)ₘ, C═(O,S)—C═(O,S); aryl, heteroaryl, heterocyclyl, cycloalkyl, alkylaryl, alkylheterocyclyl, alkylheteroaryl, arylalkyl, heterocyclylalkyl, heteroarylalkyl, and C₁-C₁₀ mono- and bicyclic cycloalkyl, wherein aryl, heteroaryl, heterocyclyl, mono- and bicyclic cycloalkyl are optionally substituted with one or more of the groups defined by R¹²;

R³⁷ and R⁴² are each independently selected from any R⁶ component;

n is an integer from 0 to 10;

m is an integer from 1 to 4;

Y″ is selected from CR⁴³, and nitrogen; and

R⁴³ is selected from any R¹ component, or

R⁴³ optionally joins with R⁴² to form a ring structure.

In a preferred embodiment, the MK-2 inhibiting compound has the structure as described just above, except wherein:

L is selected from carboxyamino, carboxyaminoalkyl, alkenyl, alkynyl, alkyl, hydrazoalkyl, arylcarbamyl, aryl, heteroaryl, arylalkyl, arylalkylamino, and alkylaryl;

n is an integer that is selected from 0, or 1;

R⁶ is optionally absent, or each R⁶ is independently selected from cycloalkyl, aryl, which can be substituted or unsubstituted, heteroaryl, which can be substituted or unsubstituted, halo, heterocyclyl, which can be substituted or unsubstituted, cyano, alkyl, alkenyl, alkynyl, alkoxy, amino, hydroxy, carboalkoxy, alkylthio, haloalkyl, carboxyl, haloalkoxy, acetyl, alkoxyaryl, hydroxyalkyl, carbamyl, cycloalkylalkyl, carboxyalkyl, alkylamino, carboxyalkenyl, nitro, cyanoalkyl, and arylalkoxy;

m is an integer selected from 0, 1, 2, 3, 4, and 5;

Y″ is selected from CR⁵, and nitrogen; and

R⁴³ is selected from any R⁶ component, or

R⁴³ optionally joins with R⁴² to form a ring structure.

The MK-2 inhibiting compounds that are described in formulas I-V, and in Tables I and II can be made by the methods that are described in the Examples below. Compounds that are not described specifically in the Examples can be made by reference to the methods used in the Examples, but with substitution of starting compounds that are suitable for the compound that is desired.

The present invention also includes a method of inhibiting mitogen activated protein kinase-activated protein kinase-2, the method comprising contacting a mitogen activated protein kinase-activated protein kinase-2 with any MK-2 inhibiting compound described above. In one embodiment, the contacting of MK-2 with an MK-2 inhibitory compound takes place inside a cell. The cell can be one of any type of organism, but is preferably an animal cell. Contacting can occur in vitro or in vivo, and the cell can be a living cell, or it can be non-living. When the contacting is carried out in vitro, the cell can be attached to other cells, or it can be a single cell, or clump of cells in suspension or on a solid medium. When the contacting is carried out in vivo, the MK-2 inhibitory compound can be administered as described below.

In one embodiment, the present invention provides a method for treating or preventing an MK-2 modulated disease or disorder in a subject, the method comprises contacting a mitogen activated protein kinase-activated protein kinase-2 in a subject with one or more of the MK-2 inhibiting compounds that are described herein. A preferred MK-2 inhibiting compound for the present method is one having the structure described by formula II.

The present invention also includes a method of inhibiting mitogen activated protein kinase-activated protein kinase-2 in a subject in need of such inhibition, the method comprising administering to the subject one or more of the MK-2 inhibiting compounds described herein.

The present invention also includes a method of preventing or treating a TNFα mediated disease or disorder in a subject, the method comprising administering to the subject an effective amount of one or more of the MK-2 inhibiting compounds described herein. In a preferred embodiment, the subject is one that is in need of such prevention or treatment.

The present methods can be practiced by the administration of any one or more of the present MK-2 inhibiting compounds. It is preferred tht the MK-2 inhibiting compound is one having an MK-2 IC₅₀ of less than about 1 μM, in an in vitro assay of MK-2 inhibitory activity, more preferred is a compound having an MK-2 IC₅₀ of less than about 0.5 μM, yet more preferred is a compound having an MK-2 IC₅₀ of less than about 0.1 μM, even more preferred is a compound having an MK-2 IC₅₀ of less than about 0.05 μM, and yet more preferred is a compound having an MK-2 IC₅₀ of less than about 0.01 μM.

It should be understood that the base forms, salts, pharmaceutically acceptable salts, and prodrugs of the compounds that are described herein, as well as isomeric forms, tautomers, racemic mixtures of the compounds, and the like, which have the same or similar activity as the compounds that are described, are to be considered to be included within the description of the compound.

The MK-2 inhibiting activity of any of the compounds described herein can be determined by any one of several methods that are well known to those having skill in the art of enzyme activity testing. One such method is described in detail in the general methods section of the examples. In addition, the efficacy of any one of the present MK-2 inhibiting compounds in therapeutic applications can be determined by testing for inhibition of TNFα production in cell culture and in animal model assays. In general, it is preferred that the MK-2 inhibiting compounds of the present invention be capable of inhibiting the production and/or the release of TNFα in cell cultures and in animal models.

In the present method, the MK-2 inhibiting compounds that are described herein can be used as inhibitors of MAPKAP kinase-2. When this inhibition is for a therapeutic purpose, one or more of the present MK-2 inhibitory compounds can be administered to a subject that is in need of MK-2 inhibition. As used herein, a “subject in need of MK-2 inhibition” is a subject who has, or who is at risk of contracting a TNFα mediated disease or disorder. TNFα mediated diseases and disorders are described in more detail below.

As described above, in an embodiment of the present method, a subject in need of prevention or treatment of a TNFα mediated disease or disorder is treated with one or more of the present MK-2 inhibiting compounds. In one embodiment, the subject is treated with an effective amount of the MK-2 inhibiting compound. The effective amount can be an amount that is sufficient for preventing or treating the TNFα mediated disease or disorder.

The MK-2 inhibiting compound that is used in the subject method can be any MK-2 inhibiting compound that is described herein.

In the subject method, the MK-2 inhibiting compound can be used in any amount that is an effective amount. It is preferred, however, that the amount of the MK-2 inhibiting compound that is administered is within a range of about 0.1 mg/day per kilogram of the subject to about 1500 mg/day/kg. It is more preferred that the amount of the compound is within a range of about 1 mg/day/kg to about 500 mg/day/kg. An amount that is within a range of about 10 mg/day/kg to about 400 mg/day/kg, is even more preferred.

When the term “about” is used herein in relation to a dosage amount of the MK-2 inhibiting compound, it is to be understood to mean an amount that is within ±10% by weight of the amount or range that is described. By way of example, “about 0.1-10 mg/day” includes all dosages within 0.9 to 11 mg/day.

In an embodiment of the present invention, a therapeutic composition is provided that contains at least one of the MK-2 inhibiting compounds that are described herein. A preferred therapeutic composition contains a therapeutically effect amount of a compound that is described by formula II.

In another embodiment of the present invention, a pharmaceutical composition that contains one or more of the present MK-2 inhibitors can be administered to a subject for the prevention or treatment of a TNFα mediated disease or disorder. The pharmaceutical composition includes an MK-2 inhibitor of the present invention and a pharmaceutically acceptable carrier. A preferred MK-2 inhibitor for use in the pharmaceutical composition is described by formula II, above.

In another embodiment, a kit can be produced that is suitable for use in the prevention or treatment of a TNFα mediated disease or disorder. The kit comprises a dosage form comprising at least one of the MK-2 inhibitors that is described herein in an amount which comprises a therapeutically effective amount.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used, the nature and severity of the illness to be treated, as well as the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of the MK-2 inhibitory compound for use in therapy which will achieve the goal of improvement in the severity of pain and inflammation and the frequency of incidence over treatment, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

The frequency of dose will depend upon the half-life of the active components of the composition. If the active molecules have a short half life (e.g. from about 2 to 10 hours) it may be necessary to give one or more doses per day. Alternatively, if the active molecules have a long half-life (e.g. from about 2 to about 15 days) it may only be necessary to give a dosage once per day, per week, or even once every 1 or 2 months. A preferred dosage rate is to administer the dosage amounts described above to a subject once per day.

For the purposes of calculating and expressing a dosage rate, all dosages that are expressed herein are calculated on an average amount-per-day basis irrespective of the dosage rate. For example, one 100 mg dosage of an MK-2 inhibitor taken once every two days would be expressed as a dosage rate of 50 mg/day. Similarly, the dosage rate of an ingredient where 50 mg is taken twice per day would be expressed as a dosage rate of 100 mg/day.

For purposes of calculation of dosage amounts, the weight of a normal adult human will be assumed to be 70 kg.

When the MK-2 inhibitor is supplied along with a pharmaceutically acceptable carrier, the pharmaceutical compositions that are described above can be formed. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the compounds and compositions of the invention are the isomeric forms and tautomers and the pharmaceutically-acceptable salts of the present MK-2 inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group IA) salts, alkaline earth metal (Group IIA) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trifluoroacetate, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The method of the present invention is useful for, but not limited to, the prevention and/or treatment of diseases and disorders that are mediated by TNFα and/or mediated by MK-2, including pain, inflammation and/or arthritis. For example, the compounds described herein would be useful for the treatment of any inflammation-related disorder described below, such as an analgesic in the treatment of pain and headaches, or as an antipyretic for the treatment of fever. The compounds described herein would also be useful for the treatment of an inflammation-related disorder in a subject suffering from such an inflammation-associated disorder.

As used herein, the terms “treating”, “treatment”, “treated”, or “to treat,” mean to alleviate symptoms, eliminate the causation either on a temporary or permanent basis. The term “treatment” includes alleviation, elimination of causation of pain and/or inflammation associated with, but not limited to, any of the diseases or disorders described herein. The terms “prevent”, “prevention”, “prevented”, or “to prevent,” mean to prevent or to slow the appearance of symptoms associated with, but not limited to, any of the diseases or disorders described herein.

In preferred embodiments, the methods and compositions of the present invention encompass the prevention and/or treatment of pain, inflammation and inflammation-related disorders.

In other preferred embodiments, the methods and compositions of the present invention encompass the treatment of any one or more of the disorders selected from the group consisting of connective tissue and joint disorders, neoplasia disorders, cardiovascular disorders, optic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

As used herein, the terms “neoplasia” and “neoplasia disorder”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as neoplasia, neoplasms or neoplasia-related disorders. Tumors are generally known in the art to be a mass of neoplasia or “neoplastic” cells. Although, it is to be understood that even one neoplastic cell is considered, for purposes of the present invention to be a neoplasm or alternatively, neoplasia.

In still other preferred embodiments, the methods and compositions of the present invention encompass the prevention and treatment of the connective tissue and joint disorders selected from the group consisting of arthritis, rheumatoid arthritis, spondyloarthopathies, gouty arthritis, lumbar spondylarthrosis, carpal tunnel syndrome, canine hip dysplasia, systemic lupus erythematosus, juvenile arthritis, osteoarthritis, tendonitis and bursitis.

In other preferred embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, skin cancer, brain cancer, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

In other preferred embodiments, the methods and compositions of the present invention encompass the prevention and treatment of the cardiovascular disorders selected from the group consisting of myocardial ischemia, hypertension, hypotension, heart arrhythmias, pulmonary hypertension, hypokalemia, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis, arteriosclerosis, atherosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, vascular diseases, migraine headaches, aplastic anemia, cardiac damage, diabetic cardiac myopathy, renal insufficiency, renal injury, renal arteriopathy, peripheral vascular disease, left ventricular hypertrophy, cognitive dysfunction, stroke, and headache.

In other preferred embodiments, the methods and compositions of the present invention encompass the prevention and treatment of the metabolic disorders selected from the group consisting of obesity, overweight, type I and type II diabetes, hypothyroidism, and hyperthyroidism.

In other preferred embodiments, the methods and compositions of the present invention encompass the prevention and treatment of the respiratory disorders selected from the group consisting of asthma, bronchitis, chronic obstructive pulmonary disease (COPD), cystic fibrosis, pulmonary edema, pulmonary embolism, pneumonia, pulmonary sarcoisosis, silicosis, pulmonary fibrosis, respiratory failure, acute respiratory distress syndrome and emphysema.

In other preferred embodiments, the methods and compositions of the present invention encompass the prevention and treatment of the angiogenesis-related disorders selected from the group consisting of angiofibroma, neovascular glaucoma, arteriovenous malformations, arthritis, osler-weber syndrome, atherosclerotic plaques, psoriasis, corneal graft neovascularization, pyogenic granuloma, delayed wound healing, retrolental fibroplasias, diabetic retinopathy, scleroderma, granulations, solid tumors, hemangioma, trachoma, hemophilic joints, vascular adhesions, hypertrophic scars, age-related macular degeneration, coronary artery disease, stroke, cancer, AIDS complications, ulcers and infertility.

In other preferred embodiments, the methods and compositions of the present invention encompass the prevention and treatment of the infectious diseases and disorders selected from the group consisting of viral infections, bacterial infections, prion infections, spirochetes infections, mycobacterial infections, rickettsial infections, chlamydial infections, parasitic infections and fungal infections.

In still further embodiments, the methods and compositions of the present invention encompass the prevention and treatment of the infectious diseases and disorders selected from the group consisting of hepatitis, HIV (AIDS), small pox, chicken pox, common cold, bacterial influenza, viral influenza, warts, oral herpes, genital herpes, herpes simplex infections, herpes zoster, bovine spongiform encephalopathy, septicemia, streptococcus infections, staphylococcus infections, anthrax, severe acquired respiratory syndrome (SARS), malaria, African sleeping sickness, yellow fever, chlamydia, botulism, canine heartworm, rocky mountain spotted fever, lyme disease, cholera, syphilis, gonorrhea, encephalitis, pneumonia, conjunctivitis, yeast infections, rabies, dengue fever, Ebola, measles, mumps, rubella, West Nile virus, meningitis, gastroenteritis, tuberculosis, hepatitis, and scarlet fever.

In other preferred embodiments, the methods and compositions of the present invention encompass the prevention and treatment of the neurological and neurodegenerative disorders selected from the group consisting of headaches, migraine headaches, Alzheimer's disease, Parkinson's disease, dementia, memory loss, senility, amyotrophy, ALS, amnesia, seizures, multiple sclerosis, muscular dystrophies, epilepsy, schizophrenia, depression, anxiety, attention deficit disorder, hyperactivity, bulimia, anorexia nervosa, anxiety, autism, phobias, spongiform encephalopathies, Creutzfeldt-Jakob disease, Huntington's Chorea, ischemia, obsessive-compulsive disorder, manic depression, bipolar disorders, drug addiction, alcoholism and smoking addiction.

In other preferred embodiments, the methods and compositions of the present invention encompass the prevention and treatment of the dermatological disorders selected from the group consisting of acne, psoriasis, eczema, burns, poison ivy, poison oak and dermatitis.

In other preferred embodiments, the methods and compositions of the present invention encompass the prevention and treatment of the surgical disorders selected from the group consisting of pain and swelling following surgery, infection following surgery and inflammation following surgery.

In other preferred embodiments, the methods and compositions of the present invention encompass the prevention and treatment of the gastrointestinal disorders selected from the group consisting of inflammatory bowel disease, irritable bowel syndrome, Crohn's disease, gastritis, irritable bowel syndrome, diarrhea, constipation, dysentery, ulcerative colitis, gastric esophageal reflux, gastric ulcers, gastric varices, ulcers, and heartburn.

In other preferred embodiments, the methods and compositions of the present invention encompass the prevention and treatment of the optic disorders selected from the group consisting of optic pain, inflammation, otorrhea, otalgia, fever, optic bleeding, Lermoyez's syndrome, Meniere's disease, vestibular neuronitis, benign paroxysmal positional vertigo, herpes zoster oticus, Ramsay Hunt's syndrome, viral neuronitis, ganglionitis, geniculate herpes, labyrinthitis, purulent labyrinthitis, viral endolymphatic labyrinthitis, perilymph fistulas, noise-induced hearing loss, presbycusis, drug-induced ototoxicity, acoustic neuromas, aerotitis media, infectious myringitis, bullous myringitis, otitis media, otitis media with effusion, acute otitis media, secretory otitis media, serous otitis media, acute mastoiditis, chronic otitis media, otitis extema, otosclerosis, squamous cell carcinoma, basal cell carcinoma, nonchromaffin paragangliomas, chemodectomas, globus jugulare tumors, globus tympanicum tumors, external otitis, perichondritis, aural eczematoid dermatitis, malignant external otitis, subperichondrial hematoma, ceruminomas, impacted cerumen, sebaceous cysts, osteomas, keloids, otalgia, tinnitus, vertigo, tympanic membrane infection, typanitis, optic furuncles, otorrhea, acute mastoiditis, petrositis, conductive and sensorineural hearing loss, epidural abscess, lateral sinus thrombosis, subdural empyema, otitic hydrocephalus, Dandy's syndrome, bullous myringitis, cerumen-impacted, diffuse external otitis, foreign bodies, keratosis obturans, optic neoplasm, otomycosis, trauma, acute barotitis media, acute eustachian tube obstruction, post-optic surgery, postsurgical otalgia, cholesteatoma, conductive and sensorineural hearing loss, epidural abscess, lateral sinus thrombosis, subdural empyema and otitic hydrocephalus.

In other preferred embodiments, the methods and compositions of the present invention encompass the prevention and treatment of the ophthalmic disorders selected from the group consisting of retinopathies, uveitis, ocular photophobia, acute injury to the eye tissue, conjunctivitis, age-related macular degeneration diabetic retinopathy, detached retina, glaucoma, vitelliform macular dystrophy type 2, gyrate atrophy of the choroid and retina, conjunctivitis, corneal infection, fuchs' dystrophy, iridocorneal endothelial syndrome, keratoconus, lattice dystrophy, map-dot-fingerprint dystrophy, ocular herpes, pterygium, myopia, hyperopia, and cataracts.

In other preferred embodiments, the methods and compositions of the present invention encompass the prevention and treatment of menstrual cramps, kidney stones, minor injuries, wound healing, vaginitis, candidiasis, sinus headaches, tension headaches, dental pain, periarteritis nodosa, thyroiditis, myasthenia gravis, multiple sclerosis, sarcoidosis, nephrotic syndrome, Bahcet's syndrome, polymyositis, gingivitis, hypersensitivity, swelling occurring after injury, closed head injury, liver disease, and endometriosis.

As used herein, the terms “TNFα mediated disease or disorder” are meant to include, without limitation, each of the symptoms or diseases that is mentioned above.

The term “subject” for purposes of treatment includes any human or animal subject who is in need of the prevention of or treatment of any one of the TNFα mediated diseases or disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

For methods of prevention, the subject is any human or animal subject, and preferably is a subject that is in need of prevention and/or treatment of a TNFα mediated diseases or disorders. The subject may be a human subject who is at risk of obtaining a TNFα mediated disease or disorder, such as those described above. The subject may be at risk due to genetic predisposition, sedentary lifestyle, diet, exposure to disorder-causing agents, exposure to pathogenic agents and the like.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

In particular, the pharmaceutical compositions of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the MK-2 inhibitors in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel MK-2 inhibitory compounds may be formulated with sweetening agents, for example glycerol, sorbitol or sucrose. Such formulations may also contain a demulcent, a preservative and flavoring and coloring agents.

The subject compositions can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-, or di-, glycerides. In addition, n-3 polyunsaturated fatty acids may find use in the preparation of injectables.

The subject compositions can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### GENERAL INFORMATION FOR PREPARATION METHODS:

Unless otherwise noted, reagents and solvents were used as received from commercial suppliers.

NMR Analysis:

Proton nuclear magnetic resonance spectra were obtained on a Varian Unity Innova 400, a Varian Unity Innova 300 a Varian Unity 300, a Bruker AMX 500 or a Bruker AV-300 spectrometer. Chemical shifts are given in ppm (δ) and coupling constants, J, are reported in Hertz. Tetramethylsilane was used as an internal standard for proton spectra and the solvent peak was used as the reference peak for carbon spectra. Mass spectra were obtained on a Perkin Elmer Sciex 100 atmospheric pressure ionization (APCI) mass spectrometer, a Finnigan LCQ Duo LCMS ion trap electrospray ionization (ESI) mass spectrometer, a PerSeptive Biosystems Mariner TOF HPLC-MS (ESI), or a Waters ZQ mass spectrometer (ESI).

Determination of MK-2 IC₅₀:

Recombinant MAPKAPK2 was phosphorylated at a concentration of 42-78 μM by incubation with 0.23 μM of active p38α in 50 mM HEPES, 0.1 mM EDTA, 10 mM magnesium acetate, and 0.25 mM ATP, pH 7.5 for one hour at 30° C.

The phosphorylation of HSP-peptide (KKKALSRQLSVAA) by MAPKAPK2 was measured using an anion exchange resin capture assay method. The reaction was carried out in 50 mM 0-glycerolphosphate, 0.04% BSA, 10 mM magnesium acetate, 2% DMSO and 0.8 mM dithiotheritol, pH 7.5 in the presence of the HSP-peptide with 0.2 μCi [γ³³PP]ATP and 0.03 mM ATP. The reaction was initiated by the addition of 15 nM MAPKAPK2 and was allowed to incubate at 30° C. for 30 min. The reaction was terminated and [γ³³P]ATP was removed from solution by the addition of 150 μl of AG 1×8 ion exchange resin in 900 mM sodium formate pH 3.0. A 50 μl aliquot of head volume was removed from the quenched reaction mixture and added to a 96-well plate, 150 μl of Microscint-40 (Packard) was added and the amount of phosphorylated-peptide was determined. Allow the Microscint to sit in the plates for 60 minutes prior to counting.

Compounds are evaluated as potential inhibitors of the MK2 kinase by measuring their effects on MK2 phosphorylation of the peptide substrate. Compounds may be screened initially at two concentrations prior to determination of IC₅₀ values. Screening results are expressed as percent inhibition at the concentrations of compound tested. For IC₅₀ value determinations, compounds are tested at six concentrations in ten-fold serial dilutions with each concentration tested in triplicate. Results are expressed as IC₅₀ values in micromolar. The assay is performed at a final concentration of 2% DMSO.

U937 Cell TNFα Release Assay

The human monocyte-like cell line, U937 (ATCC #CRL-1593.2), is cultured in RPMI1640 media with 10% heat-inactivated fetal calf serum (GIBCO), glutamine and pen/strep at 37° C. and 5% CO₂. Differentiation of U937 to monocytic/macrophage-like cells is induced by the addition of phorbol12-myristate 13-acetate (Sigma) at final concentration of 20 ng/ml to a culture of U937 cells at ˜0.5 million cells/ml and incubated for 24 hrs. The cells are centrifuged, washed with PBS and resuspended in fresh media without PMA and incubated for 24 hrs. Cells adherent to the culture flask are harvested by scraping, centrifugation, and resuspended in fresh media to 2 million cells/ml, and 0.2 ml is aliquoted to each of 96 wells in flat-bottom plate. Cells are then incubated for an additional 24 hrs to allow for recovery. The media is removed from the cells, and 0.1 ml of fresh media is added per well. 0.05 ml of serially diluted compound or control vehicle (Media with DMSO) is added to the cells. The final DMSO concentration does not exceed 1%. After 1 hr incubation, 0.05 ml of 400 ng/ml LPS (E Coli serotype 0111:B4, Sigma) in media is added for final concentration of 100 ng/ml. Cells are incubated at 37° C. for 4 hrs. After 4 hrs incubation, supernatants are harvest and assayed by ELISA for the presence of TNFα.

U937 Cell TNFα ELISA

ELISA plates (NUNC-Immuno™ Plate Maxisorb™ Surface) were coated with purified mouse monoclonal IgGl anti-human TNFα antibody (R&D Systems #MAB610; 1.25 ug/ml in sodium bicarbonate pH 8.0, 0.1 ml/well) and incubated at 4° C. Coating solution was aspirated the following day and wells were blocked with 1 mg/ml gelatin in PBS (plus 1× thimerasol) for 2 days at 4° C. Prior to using, wells were washed 3× with wash buffer (PBS with 0.05% Tween). Cultured media samples were diluted in EIA buffer (5 mg/ml bovine γ-globulin, 1 mg/ml gelatin, 1 ml/l Tween-20, 1 mg/ml thimerasol in PBS), added to wells (0.1 ml/well) in triplicate and allowed to incubate for 1.5 hr at 37° C. in a humidified chamber. Plates were again washed and 0.1 ml/well of a mixture of rabbit anti-human TNFα polyclonal antibodies in EIA buffer (1:400 dilution of Sigma #T8300, and 1:400 dilution of Calbiochem #654250) was added for 1 hr at 37° C. Plates were washed as before and peroxidase-conjugated goat anti-rabbit IgG (H+L) antibody (Jackson ImmunoResearch #111-035-144,1 ug/ml in EIA buffer, 0.1 ml/well) was added for 45 min. After final washing, plates were developed with peroxidase-ABTS solution (Kirkegaard/Perry #50-66-01, 0.1 ml/well). Enzymatic conversion of ABTS to colored product was measured after 5-30 minutes using a SpectroMax 340 spectrophotometer (Molecular Devices) at 405 nm. TNF levels were quantitated from a recombinant human TNFα (R&D Systems #210-TA-010) standard curve using a quadratic parameter fit generated by SoftMaxPRO software. ELISA sensitivity was approximately 30 pg TNF/ml. IC₅₀ values for compounds were generated using BioAssay Solver.

Lipopolysaccharide (LPS)-Induced TNFα Production.

Adult male 225-250 gram Lewis rats (Harlan Sprague-Dawley) were used. Rats were fasted 18 hr prior to oral dosing, and allowed free access to water throughout the experiment. Each treatment group consisted of 5 animals.

Compounds were prepared as a suspension in a vehicle consisting of 0.5% methylcellulose, 0.025% Tween-20 in PBS. Compounds or vehicle were orally administered in a volume of 1 ml using an 18 gauge gavage needle. LPS (E. coli serotype 0111:B4, Lot #39H4103, Cat. # L-2630, Sigma) was administered 1-4 hr later by injection into the penile vein at a dose of 1 mg/kg in 0.5 ml sterile saline. Blood was collected in serum separator tubes via cardiac puncture 1.5 hr after LPS injection, a time point corresponding to maximal TNFα production. After clotting, serum was withdrawn and stored at −20° C. until assay by ELISA (described below).

Rat LPS TNFα ELISA

ELISA plates (NUNC-Immuno™ Plate Maxisorb™ Surface) were coated with 0.1 ml per well of an Protein G purified fraction of a 2.5 ug/ml of hamster anti-mouse/rat TNFα monoclonal antibody TN19.12 (2.5 ug/ml in PBS, 0.1 ml/well). The hybridoma cell line was kindly provided by Dr. Robert Schreiber, Washington University. Wells were blocked the following day with 1 mg/ml gelatin in PBS. Serum samples were diluted in a buffer consisting of 5 mg/ml bovine y-globulin, 1 mg/ml gelatin, 1 ml/l Tween-20, 1 mg/ml thimerasol in PBS, and 0.1 ml of diluted serum was added wells in duplicate and allowed to incubate for 2 hr at 37° C. Plates were washed with PBS-Tween, and 0.1 ml per well of a 1:300 dilution of rabbit anti-mouse/rat TNFα antibody (BioSource International, Cat. #AMC3012) was added for 1.5 hr at 37° C. Plates were washed, and a 1:1000 fold dilution of peroxidase-conjugated donkey anti-rabbit IgG antibody (Jackson ImmunoResearch, Cat. #711-035-152) was added for 45 min. After washing, plates were developed with 0.1 ml of ABTS-peroxide solution (Kirkegaard/Perry, Cat. #50-66-01). Enzymatic conversion of ABTS to colored product was measured after 30 minutes using a SpectroMax 340 spectrophotometer (Molecular Devices Corp.) at 405 nm. TNF levels in serum were quantitated from a recombinant rat TNFα (BioSource International, Cat. #PRC3014.) standard curve using a quadratic parameter fit generated by SoftMaxPRO software. ELISA sensitivity was approximately 30 pg TNF/ml. Results are expressed in percent inhibition of the production of TNFα as compared to blood collected from control animals dosed only with vehicle.

### Synthesis of MK-2 Inhibiting Compounds of the Present Invention:

### EXAMPLE 1

This illustrates the procedure for the preparation of 2-(2-chloropyridin-4-yI)-1,5,6,7-tetrahydro-4H-pyrrolo[3,2-c]pyridin-4-one.

Step 1. 4-acyl-2-chloropyridine was prepared by a literature method (LaMattina, J. L. J. Heterocyclic Chem., 20:533 (1983)) from 2-chloro-4-cyanopyridine purchased from Oakwood Products, Inc.

Step 2. (Preparation of 2-bromo-1-(2-chloropyridin-4-yl)ethanone hydrobromide).

4-acyl-2-chloropyridine (3.5 g, 22.3 mmol) was dissolved in glacial acetic acid (100 mL) and treated with bromine (1.26 mL, 24.6 mmol) followed by HBr/AcOH (30% w/v, 4.4 mL, 22.3 mmol). After 15 minutes of stirring, a precipitate formed and the reaction was complete after 2-3 hours. Diluted reaction mixture with ethyl ether (100 mL) and collected the solid by filtration. The solid was washed with ethyl ether and dried under vacuum to give 2-bromo-1-(2-chloropyridin-4-yl)ethanone hydrobromide (6.51 g, 93%) as a yellow solid. ¹HNMR (400 MHz, DMSO-d₆) δ 8.62 (d, 1H), 7.96 (s, 1H), 7.83 (d, 1H), 4.99 (s, 2H). m/z (M+H): 234, 236.

Step 3. (Preparation of 2,4-dioxopiperdine).

Sodium 3-(methoxycarbonyl)-4-oxo-1,4,5,6-tetrahydropyridin-2-olate (Degussa) (50 g, 259 mmol) was partitioned between 2N aqueous hydrogen chloride and dichloromethane. The aqueous layer was extracted two additional times with dichloromethane. The organic extracts were dried over sodium sulfate, filtered and evaporated. The residue was suspended in acetonitrile (500 mL) and water (100 mL) and heated to reflux for 3 hours. The reaction mixture was cooled and evaporated. The residue was recrystallized from 1:1 ethyl acetate:hexane to provide 2,4-dioxopiperdine (19.5 g, 67%) as a white solid. ¹HNMR (400 MHz, CDCl₃) δ 7.05 (s, 1H), 3.58 (td, 2H), 3.34 (s, 2H), 2.64 (t, 2H). m/z (M+H): 114.

Step 4. (Preparation of 2-(2-chloropyridin-4-yl)-1,5,6,7-tetrahydro-4H-pyrrolo[3,2-c]pyridin-4-one).

2-bromo-1-(2-chloropyridin-4-yl)ethanone hydrobromide (6.5 g, 20.6 mmol) was combined in absolute ethanol (65 mL) with ammonium acetate (6.35 g, 82.4 mmol) and 2,4 dioxopiperdine (2.57 g, 22.7 mmol). After 30 minutes, the mixture was diluted with water (130 mL) and the mixture filtered. The resulting solid was washed with water and ethyl ether and dried under vacuum to give 2-(2-chloropyridin-4-yl)-1,5,6,7-tetrahydro-4H-pyrrolo[3,2-c]pyridin-4-one (3.15 g, 62%) as a white solid. ¹HNMR (400 MHz, DMSO-d₆) δ 12.00 (s, 1H), 8.27 (d, 1H), 7.73 (s, 1H), 7.63, (d, 1H), 7.12 (s, 1H), 7.08 (s, 1H), 3.40 (td, 2H), 2.83 (t, 2H). m/z (M+H): 248.

### EXAMPLE 2

This illustrates the production of (2-(2-thien-3-ylpyridin-4-yl)-1,5,6,7-tetrahydro-4H-pyrrolo[3,2-c]pyridin-4-one trifluoroacetate).

A suspension of 2-(2-chloropyridin-4-yl)-1,5,6,7-tetrahydro-4H-pyrrolo[3,2-c]pyridin-4-one (300 g, 1.2 mmol) in dimethylformamide (6.0 mL) was treated with 3-thiophene boronic acid (230 g, 1.8 mmol) and 2.0 M cesium carbonate (1.8 mL). The reaction was purged with nitrogen (g) 3× and then tetrakistriphenylphosphinepalladium (100 g, 0.08 mmol) was added. The reaction was then heated to 80 deg C. for 10 hrs., then cooled to room temperature and stirred for 4 hrs. The reaction mixture was then filtered through a syringe filter (0.45 μm), acidified with trifluoroacetic acid (0.5 mL), purified by prep. rpHPLC, and lyophilized to give the title compound as a yellow solid (280 g, 0.68 mmol, 57%) ¹H NMR (300 MHz, DMSO-d₆) δ 12.33 (s, 1H), 8.56 (d, J=6.0 Hz, 1H), 8.45 (s, 1H), 8.37 (s, 1H), 7.89 (d, J=5.0 Hz, 1H), 7.80 (m, 2H), 7.54 (s, 1H), 7.21 (s, 1H), 3.44 (m, 2H), 2.91 (t, J=6.7 Hz, 2H). HRMS calculated for C₁₆H₁₃N₃OS (MH⁺) 296.0852, found 296.0869. Anal. calculated for C₁₆H₁₃N₃OS.1.0 TFA1.4 H₂O C, 49.74; H, 3.89; N, 9.66. Found: C, 49.80; H, 3.76; N, 9.51.

### EXAMPLE 3

This illustrates the production of (4-[4-(4-oxo-4,5,6,7-tetrahydro-1H-pyrrolo[3,2-c]pyridin-2-yl)pyridin-2-yl]benzoic acid trifluoroacetate).

To a solution of 2-(2-chloropyridin-4-yl)-1,5,6,7-tetrahydro-4H-pyrrolo[3,2-c]pyridin-4-one (150 g, 0.60 mmol) in 2.0 mL of dimethylformamide and 2.0 mL of ethyl alcohol was added 4-carboxybenzene boronic acid (151 g, 0.90 mmol), 2.0 M cesium carbonate (0.9 mL), and tetrakistriphenylphosphinepalladium (0) (50 mg, 0.04 mmol). The reaction was heated to 80 deg. C. for 16 hours. The reaction was cooled to room temperature, filtered through a syring filter (0.45 μm) and purified by prep rpHPLC, and lyophilized to give the title compound as a yellow solid (110 g, 0.25 mmol, 42%). ¹H NMR (300 MHz, DMSO-d₆) δ 12.25 (s, 1H), 8.64 (d, J=5.4 Hz, 1H), 8.39 (s, 1H), 8.23 (d, J=8.5 Hz, 2H), 8.10 (d, J=8.3 Hz, 2H), 7.81 (d, J=5.2 Hz, 1H), 7.40 (s, 1H), 7.16 (s, 1H), 3.43 (t, J=6.1 Hz, 2H), 2.90 (t, J=6.6 Hz, 2H). HRMS calculated for C₁₉H₁₅N₃O₃ (MH⁺) 334.1186, found 334.1188. Anal. calculated for C₁₉H₁₅N₃O₃.1.2 TFA 1.6 H2O C, 51.51; H, 3.91; N, 8.42. Found: C, 51.59; H, 3.95; N, 8.44.

The following compounds were prepared from 2-(2-chloropyridin-4-yl)-1,5,6,7-tetrahydro-4H-pyrrolo[3,2-c]pyridin-4-one as described for Example 2.

| Example | | Calculated | Found |
|---|---|---|---|
| No. | Compound Name | (m + H) | (m + H) |
| 4 | 2-[2-(3-isopropylphenyl)pyridin- | 332.1757 | 332.1763 |
| | 4-yl]-1,5,6,7-tetrahydro-4H- | | |
| | pyrrolo[3,2-c]pyridin-4-one | | |
| | trifluoroacetate | | |
| 5 | 2-(2-quinolin-3-ylpyridin-4-yl)- | 341.1397 | 341.1396 |
| | 1,5,6,7-tetrahydro-4H-pyrrolo[3, | | |
| | 2-c]pyridin-4-one trifluoroacetate | | |
| 6 | 2-[2-(4-methoxyphenyl)pyridin- | 320.1394 | 320.1405 |
| | 4-yl]-1,5,6,7-tetrahydro-4H- | | |
| | pyrrolo[3,2-c]pyridin-4-one | | |
| | trifluoroacetate | | |
| 7 | 2-(2,3′-bipyridin-4-yl)-1,5,6, | 291.124 | 291.1242 |
| | 7-tetrahydro-4H-pyrrolo[3, | | |
| | 2-c]pyridin-4-one trifluoroacetate | | |
| 8 | 2-[2-(3-methoxyphenyl)pyridin- | 320.1394 | 320.142 |
| | 4-yl]-1,5,6,7-tetrahydro-4H- | | |
| | pyrrolo[3,2-c]pyridin-4-one | | |
| | trifluoroacetate | | |
| 9 | 2-{2-[3- | 374.1111 | 374.1133 |
| | (trifluoromethoxy)phenyl]pyridin- | | |
| | 4-yl}-1,5,6,7-tetrahydro-4H- | | |
| | pyrrolo[3,2-c]pyridin-4-one | | |
| | trifluoroacetate | | |
| 10 | 2-[2-(4-isopropylphenyl)pyridin- | 332.1757 | 332.1731 |
| | 4-yl]-1,5,6,7-tetrahydro-4H- | | |
| | pyrrolo[3,2-c]pyridin-4-one | | |
| | trifluoroacetate | | |
| 11 | 2-{2-[4- | 333.171 | 333.1685 |
| | (dimethylamino)phenyl]pyridin- | | |
| | 4-yl}-1,5,6,7-tetrahydro-4H- | | |
| | pyrrolo[3,2-c]pyridin-4-one | | |
| | trifluoroacetate | | |
| 12 | 2-[2-(4-chlorophenyl)pyridin- | 324.0898 | 324.0892 |
| | 4-yl]-1,5,6,7-tetrahydro-4H- | | |
| | pyrrolo[3,2-c]pyridin-4-one | | |
| | trifluoroacetate | | |
| 13 | 2-[2-(3,4-dimethylphenyl)pyridin- | 318.1601 | 318.1609 |
| | 4-yl]-1,5,6,7-tetrahydro-4H- | | |
| | pyrrolo[3,2-c]pyridin-4-one | | |
| | trifluoroacetate | | |
| 14 | 2-[2-(4-fluorophenyl)pyridin- | 308.1194 | 308.1186 |
| | 4-yl]-1,5,6,7-tetrahydro-4H- | | |
| | pyrrolo[3,2-c]pyridin-4-one | | |
| | trifluoroacetate | | |
| 15 | 2-[2-(4-chloro-3- | 338.1055 | 338.1075 |
| | methylphenyl)pyridin-4-yl]-1,5, | | |
| | 6,7-tetrahydro-4H-pyrrolo[3, | | |
| | 2-c]pyridin-4-one trifluoroacetate | | |
| 16 | 2-{2-[4- | 374.1111 | 374.1105 |
| | (trifluoromethoxy)phenyl]pyridin- | | |
| | 4-yl}-1,5,6,7-tetrahydro-4H- | | |
| | pyrrolo[3,2-c]pyridin-4-one | | |
| | trifluoroacetate | | |
| 17 | 2-{2-[4- | 396.1707 | 396.1691 |
| | (benzyloxy)phenyl]pyridin- | | |
| | 4-yl}-1,5,6,7-tetrahydro-4H- | | |
| | pyrrolo[3,2-c]pyridin-4-one | | |
| 18 | 2-[2-(3-fluoro-4- | 338.1299 | 338.1305 |
| | methoxyphenyl)pyridin-4-yl]- | | |
| | 1,5,6,7-tetrahydro-4H-pyrrolo[3, | | |
| | 2-c]pyridin-4-one trifluoroacetate | | |
| 19 | 2-[2-(3-fluoro-4- | 322.135 | 322.1361 |
| | methylphenyl)pyridin-4-yl]-1, | | |
| | 5,6,7-tetrahydro-4H-pyrrolo[3, | | |
| | 2-c]pyridin-4-one trifluoroacetate | | |
| 20 | 2-[2-(4-propylphenyl)pyridin- | 332.1757 | 332.1732 |
| | 4-yl]-1,5,6,7-tetrahydro-4H- | | |
| | pyrrolo[3,2-c]pyridin-4-one | | |
| | trifluoroacetate | | |
| 21 | 2-[2-(4-butylphenyl)pyridin- | 346.1914 | 346.1916 |
| | 4-yl]-1,5,6,7-tetrahydro-4H- | | |
| | pyrrolo[3,2-c]pyridin-4-one | | |
| | trifluoroacetate | | |
| 22 | 2-[2-(4-butoxyphenyl)pyridin- | 362.1863 | 362.1886 |
| | 4-yl]-1,5,6,7-tetrahydro-4H- | | |
| | pyrrolo[3,2-c]pyridin-4-one | | |
| | trifluoroacetate | | |
| 23 | 2-[2-(4-ethoxyphenyl)pyridin- | 334.155 | 334.1555 |
| | 4-yl]-1,5,6,7-tetrahydro-4H- | | |
| | pyrrolo[3,2-c]pyridin-4-one | | |
| | trifluoroacetate | | |
| 24 | {4-[4-(4-oxo-4,5,6, | 329.1397 | 329.1414 |
| | 7-tetrahydro-1H-pyrrolo[3, | | |
| | 2-c]pyridin-2-yl)pyridin- | | |
| | 2-yl]phenyl}acetonitrile | | |
| 25 | 2-{2-[4- | 358.1162 | 358.1156 |
| | (trifluoromethyl)phenyl]pyridin- | | |
| | 4-yl}-1,5,6,7-tetrahydro-4H- | | |
| | pyrrolo[3,2-c]pyridin-4-one | | |
| 26 | 2-[2-(3-chlorophenyl)pyridin- | 324.0898 | 324.0899 |
| | 4-yl]-1,5,6,7-tetrahydro-4H- | | |
| | pyrrolo[3,2-c]pyridin-4-one | | |
| | trifluoroacetate | | |
| 27 | 2-[2-(3,5-dichlorophenyl)pyridin- | 358.0508 | 358.0492 |
| | 4-yl]-1,5,6,7-tetrahydro-4H- | | |
| | pyrrolo[3,2-c]pyridin-4-one | | |
| | trifluoroacetate | | |
| 28 | 2-[2-(1,3-benzodioxol- | 334.1186 | 334.1184 |
| | 5-yl)pyridin-4-yl]-1,5,6, | | |
| | 7-tetrahydro-4H-pyrrolo[3, | | |
| | 2-c]pyridin-4-one | | |
| 29 | 2-[2-(2-naphthyl)pyridin-4-yl]-1, | 340.1444 | 340.1114 |
| | 5,6,7-tetrahydro-4H-pyrrolo[3, | | |
| | 2-c]pyridin-4-one trifluoroacetate | | |
| 30 | 3-[4-(4-oxo-4,5,6, | 318.1237 | 318.1227 |
| | 7-tetrahydro-1H-pyrrolo[3, | | |
| | 2-c]pyridin-2-yl)pyridin- | | |
| | 2-yl]benzaldehyde | | |
| 31 | 2-(2-quinolin-5-ylpyridin-4-yl)- | 341.1397 | 341.1419 |
| | 1,5,6,7-tetrahydro-4H-pyrrolo[3, | | |
| | 2-c]pyridin-4-one. | | |
| 32 | 2-[2-(4-acetylphenyl)pyridin- | 331.1321 | 332.19 |
| | 4-yl]-1,5,6, | | |
| | 7-tetrahydro-4H-pyrrolo[3, | | |
| | 2-c]pyridin-4-one trifluoroacetate | | |
| 33 | 2-(2-quinolin-8-ylpyridin-4-yl)-1, | 341.1397 | 341.1413 |
| | 5,6,7-tetrahydro-4H-pyrrolo[3, | | |
| | 2-c]pyridin-4-one trifluoroacetate | | |
| 34 | 2-[2-(1,2,3,4-tetrahydroquinolin- | 345.171 | 345.1737 |
| | 8-yl)pyridin-4-yl]-1,5,6, | | |
| | 7-tetrahydro-4H-pyrrolo[3, | | |
| | 2-c]pyridin-4-one trifluoroacetate | | |
| 35 | 2-(2-isoquinolin-4-ylpyridin- | 341.1397 | 341.1418 |
| | 4-yl)-1,5,6,7-tetrahydro-4H- | | |
| | pyrrolo[3,2-c]pyridin- | | |
| | 4-one trifluoroacetate | | |
| 36 | 2-[3-(1,8-naphthyridin- | 341.1397 | 341.1412 |
| | 2-yl)phenyl]-1,5,6, | | |
| | 7-tetrahydro-4H-pyrrolo[3, | | |
| | 2-c]pyridin-4-one trifluoroacetate | | |
| 37 | 2-[3-(5,6,7,8-tetrahydro-1,8- | 345.171 | 345.1731 |
| | naphthyridin-2-yl)phenyl]-1,5,6, | | |
| | 7-tetrahydro-4H-pyrrolo[3, | | |
| | 2-c]pyridin-4-one trifluoroacetate | | |
| 38 | 2-[2-(2-aminophenyl)pyridin- | 305.1397 | 305.1412 |
| | 4-yl]-1,5,6,7-tetrahydro-4H- | | |
| | pyrrolo[3,2-c]pyridin- | | |
| | 4-one trifluoroacetate | | |
| 39 | 2-{2-[5-(hydroxymethyl)thien- | 325.0885 | 326.1 |
| | 2-yl]pyridin-4-yl}-1,5,6, | | |
| | 7-tetrahydro-4H-pyrrolo[3, | | |
| | 2-c]pyridin-4-one | | |
| 40 | 2-[2-(3,5-difluoro-4- | 341.0976 | 342.1 |
| | hydroxyphenyl)pyridin-4-yl]- | | |
| | 1,5,6,7-tetrahydro-4H-pyrrolo[3, | | |
| | 2-c]pyridin-4-one trifluoroacetate | | |
| 41 | 2-[2-(3,5-difluoro-4- | 355.1132 | 356.1 |
| | methoxyphenyl)pyridin-4-yl]-1, | | |
| | 5,6,7-tetrahydro-4H-pyrrolo[3, | | |
| | 2-c]pyridin-4-one trifluoroacetate | | |
| 42 | 2-[2-(2-methoxypyrimidin- | 321.1226 | 322.1 |
| | 5-yl)pyridin-4-yl]-1,5,6, | | |
| | 7-tetrahydro-4H-pyrrolo[3, | | |
| | 2-c]pyridin-4-one trifluoroacetate | | |
| 43 | 2-[2-(1-benzyl-1H-pyrazol- | 369.159 | 370.2 |
| | 4-yl)pyridin-4-yl]-1,5,6, | | |
| | 7-tetrahydro-4H-pyrrolo[3, | | |
| | 2-c]pyridin-4-one trifluoroacetate | | |
| 44 | 2-[2-(4-hydroxy-3,5- | 333.1477 | 334.2 |
| | dimethylphenyl)pyridin-4-yl]- | | |
| | 1,5,6,7-tetrahydro-4H-pyrrolo[3, | | |
| | 2-c]pyridin-4-one trifluoroacetate | | |
| 45 | N,N-dimethyl-4-[4-(4-oxo-4,5, | 396.1256 | 397.1 |
| | 6,7-tetrahydro-1H-pyrrolo[3, | | |
| | 2-c]pyridin-2-yl)pyridin- | | |
| | 2-yl]benzenesulfonamide | | |
| | trifluoroacetate | | |
| 46 | 2-(2-{1-[(4- | 482.1413 | 483.2 |
| | methylphenyl)sulfonyl]-1H-indol- | | |
| | 3-yl}pyridin-4-yl)-1,5,6, | | |
| | 7-tetrahydro-4H-pyrrolo[3, | | |
| | 2-c]pyridin-4-one trifluoroacetate | | |
| 47 | tert-butyl 2,6-di-tert-butyl-4- | 517.2941 | 518.3 |
| | [4-(4-oxo-4,5,6,7-tetrahydro-1H- | | |
| | pyrrolo[3,2-c]pyridin-2-yl)pyridin- | | |
| | 2-yl]phenyl carbonate | | |
| 48 | 2-{2-[2-fluoro-4-(morpholin- | 456.1268 | 457.2 |
| | 4-ylsulfonyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 49 | 2-[2-(1-methyl-1H-indol- | 342.1481 | 343.3 |
| | 2-yl)pyridin-4-yl]-1,5,6, | | |
| | 7-tetrahydro-4H-pyrrolo[3, | | |
| | 2-c]pyridin-4-one trifluoroacetate | | |
| 50 | 2-[2-(2-fluoro-4- | 323.107 | 324.1 |
| | hydroxyphenyl)pyridin-4-yl]- | | |
| | 1,5,6,7-tetrahydro-4H- | | |
| | pyrrolo[3,2-c]pyridin-4-one | | |
| 51 | 2-[2-(3,5-di-tert-butyl-4- | 417.2416 | 418.3 |
| | hydroxyphenyl)pyridin-4-yl]- | | |
| | 1,5,6,7-tetrahydro-4H-pyrrolo[3, | | |
| | 2-c]pyridin-4-one trifluoroacetate | | |
| 52 | 3-[4-(4-oxo-4,5,6,7-tetrahydro-1H- | 315.124 | 315.125 |
| | pyrrolo[3,2-c]pyridin- | | |
| | 2-yl)pyridin-2-yl]benzonitrile | | |
| | trifluoroacetate | | |
| 53 | 2-[2-(3,5-difluorophenyl)pyridin- | 326.1099 | 326.1132 |
| | 4-yl]-1,5,6,7-tetrahydro-4H- | | |
| | pyrrolo[3,2-c]pyridin-4-one | | |
| | trifluoroacetate | | |
| 54 | 2-{2-[4- | 336.1165 | 336.1178 |
| | (methylthio)phenyl]pyridin- | | |
| | 4-yl}-1,5,6,7-tetrahydro-4H- | | |
| | pyrrolo[3,2-c]pyridin-4-one | | |
| | trifluoroacetate | | |
| 55 | 2-[2-(3-fluorophenyl)pyridin- | 308.1194 | 308.1162 |
| | 4-yl]-1,5,6,7-tetrahydro-4H- | | |
| | pyrrolo[3,2-c]pyridin-4-one | | |
| | trifluoroacetate | | |
| 56 | 2-[2-(1-benzofuran-2-yl)pyridin- | 330.1237 | 330.1267 |
| | 4-yl]-1,5,6,7-tetrahydro-4H- | | |
| | pyrrolo[3,2-c]pyridin-4-one | | |
| | trifluoroacetate | | |
| 57 | 2-[2-(1-benzothien-2-yl)pyridin- | 346.1009 | 346.1043 |
| | 4-yl]-1,5,6,7-tetrahydro-4H- | | |
| | pyrrolo[3,2-c]pyridin-4-one | | |
| | trifluoroacetate | | |
| 58 | 2-[2-(3,4-dichlorophenyl)pyridin- | 358.0508 | 358.0536 |
| | 4-yl]-1,5,6,7-tetrahydro-4H- | | |
| | pyrrolo[3,2-c]pyridin-4-one | | |
| | trifluoroacetate | | |
| 59 | 2-[2-(3,4-difluorophenyl)pyridin- | 326.1099 | 326.1086 |
| | 4-yl]-1,5,6,7-tetrahydro-4H- | | |
| | pyrrolo[3,2-c]pyridin-4-one | | |
| | trifluoroacetate | | |
| 60 | 2-[2-(3-acetylphenyl)pyridin- | 332.1394 | 332.1388 |
| | 4-yl]-1,5,6,7-tetrahydro-4H- | | |
| | pyrrolo[3,2-c]pyridin-4-one | | |
| | trifluoroacetate | | |
| 61 | 2-{2-[3- | 358.1162 | 358.1158 |
| | (trifluoromethyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 62 | 4-[4-(4-oxo-4,5,6,7-tetrahydro- | 315.124 | 315.1235 |
| | 1H-pyrrolo[3,2-c]pyridin- | | |
| | 2-yl)pyridin-2-yl]benzonitrile | | |
| | trifluoroacetate | | |
| 63 | 2-(2-phenylpyridin-4-yl)-1,5,6, | 290.1288 | 290.1319 |
| | 7-tetrahydro-4H-pyrrolo[3, | | |
| | 2-c]pyridin-4-one trifluoroacetate | | |
| 64 | 2-(2-pyrimidin-5-ylpyridin-4-yl)- | 292.1193 | 292.1179 |
| | 1,5,6,7-tetrahydro-4H-pyrrolo[3, | | |
| | 2-c]pyridin-4-one trifluoroacetate | | |
| 65 | 2-[2-(2-fluorophenyl)pyridin- | 308.1194 | 308.1224 |
| | 4-yl]-1,5,6,7-tetrahydro-4H- | | |
| | pyrrolo[3,2-c]pyridin-4-one | | |
| | trifluoroacetate | | |
| 66 | 2-[2-(2,4-difluorophenyl)pyridin- | 326.1099 | 326.1136 |
| | 4-yl]-1,5,6,7-tetrahydro-4H- | | |
| | pyrrolo[3,2-c]pyridin-4-one | | |
| | trifluoroacetate | | |
| 67 | 2-[2-(3-nitrophenyl)pyridin- | 335.1139 | 335.1137 |
| | 4-yl]-1,5,6,7-tetrahydro-4H- | | |
| | pyrrolo[3,2-c]pyridin-4-one | | |
| | trifluoroacetate | | |
| 68 | 2-[2-(4-nitrophenyl)pyridin- | 335.1139 | 335.1151 |
| | 4-yl]-1,5,6,7-tetrahydro-4H- | | |
| | pyrrolo[3,2-c]pyridin-4-one | | |
| | trifluoroacetate | | |
| 69 | 2-[2-(2-chlorophenyl)pyridin- | 324.0898 | 324.0892 |
| | 4-yl]-1,5,6,7-tetrahydro-4H- | | |
| | pyrrolo[3,2-c]pyridin-4-one | | |
| | trifluoroacetate | | |
| 70 | N-cyclopentyl-4-[4-(4-oxo-4,5,6, | 401.1972 | 401.1982 |
| | 7-tetrahydro-1H-pyrrolo[3, | | |
| | 2-c]pyridin-2-yl)pyridin- | | |
| | 2-yl]benzamide | | |
| 71 | N-benzyl-4-[4-(4-oxo-4,5,6, | 423.1816 | 423.1811 |
| | 7-tetrahydro-1H-pyrrolo[3, | | |
| | 2-c]pyridin-2-yl)pyridin- | | |
| | 2-yl]benzamide trifluoroacetate | | |
| 72 | N-cyclopropyl-4-[4-(4-oxo-4,5,6, | 373.1659 | 373.1638 |
| | 7-tetrahydro-1H-pyrrolo[3, | | |
| | 2-c]pyridin-2-yl)pyridin- | | |
| | 2-yl]benzamide | | |
| 73 | N-cyclohexyl-4-[4-(4-oxo-4,5,6, | 415.2129 | 415.2156 |
| | 7-tetrahydro-1H-pyrrolo[3, | | |
| | 2-c]pyridin-2-yl)pyridin- | | |
| | 2-yl]benzamide trifluoroacetate | | |
| 74 | 2-[2-(4-hydroxyphenyl)pyridin- | 306.1237 | 306.1219 |
| | 4-yl]-1,5,6,7-tetrahydro-4H- | | |
| | pyrrolo[3,2-c]pyridin-4-one | | |
| 75 | methyl 4-[4-(4-oxo-4,5,6, | 348.1343 | 348.1328 |
| | 7-tetrahydro-1H-pyrrolo[3, | | |
| | 2-c]pyridin-2-yl)pyridin- | | |
| | 2-yl]benzoate trifluoroacetate | | |
| 76 | 2-[2-(3-hydroxyphenyl)pyridin- | 306.1237 | 306.1224 |
| | 4-yl]-1,5,6,7-tetrahydro-4H- | | |
| | pyrrolo[3,2-c]pyridin-4-one | | |
| | trifluoroacetate | | |
| 77 | 2-[2-(1H-indol-5-yl)pyridin- | 329.1397 | 329.14 |
| | 4-yl]-1,5,6,7-tetrahydro-4H- | | |
| | pyrrolo[3,2-c]pyridin-4-one | | |
| | trifluoroacetate | | |
| 78 | 2-[2-(4-aminophenyl)pyridin- | 305.1397 | 305.1385 |
| | 4-yl]-1,5,6,7-tetrahydro-4H- | | |
| | pyrrolo[3,2-c]pyridin-4-one | | |
| | trifluoroacetate | | |
| 79 | 2-{2-[4- | 320.1394 | 320.1393 |
| | (hydroxymethyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 80 | 2-{2-[3-(2- | 334.155 | 334.1541 |
| | hydroxyethyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 81 | 2-(2,4′-bipyridin-4-yl)-1,5,6, | 291.124 | 291.1243 |
| | 7-tetrahydro-4H-pyrrolo[3, | | |
| | 2-c]pyridin-4-one trifluoroacetate | | |
| 82 | 3-[4-(4-oxo-4,5,6,7-tetrahydro- | 333.1346 | 333.1376 |
| | 1H-pyrrolo[3,2-c]pyridin- | | |
| | 2-yl)pyridin-2-yl]benzamide | | |
| | trifluoroacetate | | |
| 83 | 2-[2-(1-benzothien-3-yl)pyridin- | 346.1009 | 346.0997 |
| | 4-yl]-1,5,6,7-tetrahydro-4H- | | |
| | pyrrolo[3,2-c]pyridin-4-one | | |
| | trifluoroacetate | | |
| 84 | 2-{2-[3- | 320.1394 | 320.1394 |
| | (hydroxymethyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 85 | 2-[2-(2,3-difluorophenyl)pyridin- | 326.1099 | 326.1117 |
| | 4-yl]-1,5,6,7-tetrahydro-4H- | | |
| | pyrrolo[3,2-c]pyridin-4-one | | |
| | trifluoroacetate | | |
| 86 | 2-[2-(4-ethylphenyl)pyridin- | 318.1601 | 318.162 |
| | 4-yl]-1,5,6,7-tetrahydro-4H- | | |
| | pyrrolo[3,2-c]pyridin-4-one | | |
| | trifluoroacetate | | |
| 87 | 2-{2-[3- | 333.171 | 333.1705 |
| | (dimethylamino)phenyl]pyridin- | | |
| | 4-yl}-1,5,6,7-tetrahydro-4H- | | |
| | pyrrolo[3,2-c]pyridin-4-one | | |
| | trifluoroacetate | | |
| 88 | 3-[4-(4-oxo-4,5,6,7-tetrahydro- | 334.1186 | 334.1153 |
| | 1H-pyrrolo[3,2-c]pyridin- | | |
| | 2-yl)pyridin-2-yl]benzoic acid | | |
| | trifluoroacetate | | |
| 89 | 2-{2-[4-(piperidin- | 401.1972 | 401.1998 |
| | 1-ylcarbonyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 90 | 2-[2-(2-hydroxyphenyl)pyridin- | 306.1237 | 306.125 |
| | 4-yl]-1,5,6,7-tetrahydro-4H- | | |
| | pyrrolo[3,2-c]pyridin-4-one | | |
| | trifluoroacetate | | |
| 91 | 2-[2-(2-methoxyphenyl)pyridin- | 320.1394 | 320.1393 |
| | 4-yl]-1,5,6,7-tetrahydro-4H- | | |
| | pyrrolo[3,2-c]pyridin-4-one | | |
| | trifluoroacetate | | |
| 92 | (2E)-3-{4-[4-(4-oxo-4,5,6, | 360.1343 | 360.1319 |
| | 7-tetrahydro-1H-pyrrolo[3, | | |
| | 2-c]pyridin-2-yl)pyridin- | | |
| | 2-yl]phenyl}prop-2-enoic | | |
| | acid trifluoroacetate | | |
| 93 | 2-{2-[3- | 368.1063 | 368.1049 |
| | (methylsulfonyl)phenyl]pyridin- | | |
| | 4-yl}-1,5,6,7-tetrahydro-4H- | | |
| | pyrrolo[3,2-c]pyridin-4- | | |
| | one trifluoroacetate | | |
| 94 | 2-{2-[3- | 352.1114 | 352.1093 |
| | (methylsulfinyl)phenyl]pyridin- | | |
| | 4-yl}-1,5,6,7-tetrahydro-4H- | | |
| | pyrrolo[3,2-c]pyridin-4- | | |
| | one trifluoroacetate | | |
| 95 | ethyl 3-[4-(4-oxo-4,5,6, | 362.1499 | 362.1489 |
| | 7-tetrahydro-1H-pyrrolo[3, | | |
| | 2-c]pyridin-2-yl)pyridin- | | |
| | 2-yl]benzoate trifluoroacetate | | |
| 96 | N-cyclopentyl-3-[4-(4-oxo-4,5, | 401.1972 | 401.2004 |
| | 6,7-tetrahydro-1H-pyrrolo[3, | | |
| | 2-c]pyridin-2-yl)pyridin- | | |
| | 2-yl]benzamide trifluoroacetate | | |
| 97 | 2-[2-(3,4,5- | 344.1017 | 344.1005 |
| | Trifluorophenyl)pyridin-4-yl]- | | |
| | 1,5,6,7-tetrahydro-4H-pyrrolo[3, | | |
| | 2-c]pyridin-4-one trifluoroacetate | | |
| 98 | 2-[2-(4-Hydroxy-2- | 320.1394 | 320.14 |
| | methylphenyl)pyridin-4-yl]- | | |
| | 1,5,6,7-tetrahydro-4H-pyrrolo[3, | | |
| | 2-c]pyridin-4-one trifluoroacetate | | |
| 99 | 2-[2-(5-acetyl-2- | 350.1305 | 350 |
| | fluorophenyl)pyridin-4-yl]-1,5, | | |
| | 6,7-tetrahydro-4H-pyrrolo[3, | | |
| | 2-c]pyridin-4-one trifluoroacetate | | |
| 100 | 2-[2-(1,1′-biphenyl-3-yl)pyridin- | 366.1606 | 366 |
| | 4-yl]-1,5,6,7-tetrahydro-4H- | | |
| | pyrrolo[3,2-c]pyridin-4-one | | |
| 101 | 2-[2-(2- | 330.1606 | 330 |
| | phenylcyclopropyl)pyridin-4-yl]-1, | | |
| | 5,6,7-tetrahydro-4H-pyrrolo[3, | | |
| | 2-c]pyridin-4-one | | |
| 102 | 2-{2-[4- | 319.1559 | 319 |
| | (aminomethyl)phenyl]pyridin- | | |
| | 4-yl}-1,5,6,7-tetrahydro-4H- | | |
| | pyrrolo[3,2-c]pyridin-4-one | | |
| 103 | 2-[2-(1H-pyrazol-4-yl)pyridin- | 280.1193 | 280.1202 |
| | 4-yl]-1,5,6,7-tetrahydro-4H- | | |
| | pyrrolo[3,2-c]pyridin-4-one | | |
| | trifluoroacetate | | |
| 104 | 2-{2-[4- | 320.1399 | 320.2 |
| | (hydroxymethyl)phenyl]pyridin- | | |
| | 4-yl}-1,5,6,7-tetrahydro-4H- | | |
| | pyrrolo[3,2-c]pyridin-4-one | | |
| 105 | 2-[2-(3,5- | 318.1601 | 318.1622 |
| | dimethylphenyl)pyridin-4-yl]-1, | | |
| | 5,6,7-tetrahydro-4H-pyrrolo[3, | | |
| | 2-c]pyridin-4-one trifluoroacetate | | |
| 106 | 2-[2-(3-tert-butyl-5- | 360.207 | 360.2039 |
| | methylphenyl)pyridin-4-yl]- | | |
| | 1,5,6,7-tetrahydro-4H-pyrrolo[3, | | |
| | 2-c]pyridin-4-one trifluoroacetate | | |
| 107 | 2,4-dimethoxypyrimidin- | 352.1404 | 352.1384 |
| | 5-yl)pyridin-4-yl]-1,5,6, | | |
| | 7-tetrahydro-4H-pyrrolo[3, | | |
| | 2-c]pyridin-4-one trifluoroacetate | | |
| 108 | 2-{2-[3,5- | 426.1036 | 426.1015 |
| | bis(trifluoromethyl)phenyl]pyridin- | | |
| | 4-yl}-1,5,6,7-tetrahydro-4H- | | |
| | pyrrolo[3,2-c]pyridin-4-one | | |
| | trifluoroacetate | | |

### EXAMPLE 109

This illustrates the production of 2-[2-(2,3-dihydro-1,4-benzodioxin-6-yl)pyridin-4-yl]-1,5,6,7-tetrahydro-4H-pyrrolo[3,2-c]pyridin-4-one.

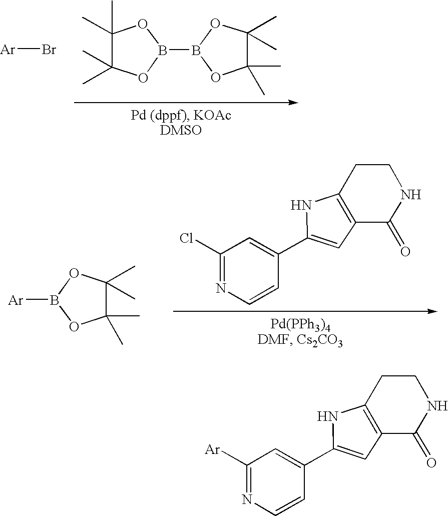

This example illustrates the general procedure for the production of boronic esters from bromides as reported by Tatsuo (J. Org. Chem. 1995, 60, 23, 7508.). A solution of 6-bromo-2,3-dihydro-1,4-benzodioxine (Lancaster, 1.6 g, 7.4 mmol) Pd(dppf)Cl₂ (180 g, 2 mol%), potassium acetate (2.17 g, 3.0 equiv.), pinacole diborane (2.06 g, 1.1 equiv.) in dimethylsulfoxide (50 mL) was heated to 80° C. for 16 hours. The mixture was cooled to room temperature, diluted with ethyl acetate (100 mL) and water (50 mL) and filtered through celite washing with ethyl acetate. The organic layer was washed with water (4×50 mL) dried over sodium sulfate and evaporated to yield the crude boronic ester as a dark oil. The crude boronic ester was reacted with 2-(2-chloropyridin-4-yl)-1,5,6,7-tetrahydro-4H-pyrrolo[3,2-c]pyridin-4-one as described for Example 2 to yield the title compound. (60% overall) ¹H NMR (400 MHz, MeOD-d₄): δ 8.39, d, J=5.4, 1H; 7.87, s, 1H; 7.44-7.40, m, 3H; 7.04, s, 1 H: 6.89, d, J=8.3, 1H) 4.25, s, 4H; 3.54, t, J=7.0, 2H; 2.91, t, J=7.0, 2H. m/z 348 (M+H) Calculated for C₂₀H₁₇N₃O+H: 348.1343. Found: 348.1330.

The following examples were prepared by the same method as described for Example 109.

| Example | | Calculated | Found |
|---|---|---|---|
| No. | Compound Name | (m + H) | m + H |
| 110 | 2-(6′-fluoro-2,3′-bipyridin-4-yl)- | 309.1146 | 309.1144 |
| | 1,5,6,7-tetrahydro-4H-pyrrolo[3, | | |
| | 2-c]pyridin-4-one trifluoroacetate | | |
| 111 | 2-(6′-amino-2,3′-bipyridin-4-yl)- | 306.1349 | 306.133 |
| | 1,5,6,7-tetrahydro-4H-pyrrolo[3, | | |
| | 2-c]pyridin-4-one trifluoroacetate | | |
| 112 | 2-[2-(6-hydroxy-2- | 356.1394 | 356.1362 |
| | naphthyl)pyridin-4-yl]-1,5,6, | | |
| | 7-tetrahydro-4H-pyrrolo[3, | | |
| | 2-c]pyridin-4-one trifluoroacetate | | |
| 113 | 2-(6′-methoxy-2,3′-bipyridin-4-yl)- | 321.1346 | 321.1358 |
| | 1,5,6,7-tetrahydro-4H-pyrrolo[3, | | |
| | 2-c]pyridin-4-one trifluoroacetate | | |
| 114 | 2-{2-[4- | 344.1757 | 344.1734 |
| | (cyclopropylmethyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 115 | 2-[2-(2-fluoro-4- | 322.135 | 322.1373 |
| | methylphenyl)pyridin-4-yl]-1,5,6, | | |
| | 7-tetrahydro-4H-pyrrolo[3, | | |
| | 2-c]pyridin-4-one trifluoroacetate | | |
| 116 | 2-[2-(4-chloro-3- | 342.0804 | 342.0819 |
| | fluorophenyl)pyridin-4-yl]-1,5,6, | | |
| | 7-tetrahydro-4H-pyrrolo[3, | | |
| | 2-c]pyridin-4-one trifluoroacetate | | |
| 117 | 2-[2-(6-methoxy-2- | 370.155 | 370.1551 |
| | naphthyl)pyridin-4-yl]-1,5,6, | | |
| | 7-tetrahydro-4H-pyrrolo[3, | | |
| | 2-c]pyridin-4-one trifluoroacetate | | |
| 118 | 2-[2-(4-chloro-2- | 342.0804 | 342.0822 |
| | fluorophenyl)pyridin-4-yl]-1,5,6, | | |
| | 7-tetrahydro-4H-pyrrolo[3, | | |
| | 2-c]pyridin-4-one trifluoroacetate | | |
| 119 | 2-[2-(3-fluoro-4- | 324.1143 | 324.1137 |
| | hydroxyphenyl)pyridin-4-yl]-1,5,6, | | |
| | 7-tetrahydro-4H-pyrrolo[3, | | |
| | 2-c]pyridin-4-one trifluoroacetate | | |
| 120 | 2-fluoro-4-[4-(4-oxo-4,5,6, | 333.1146 | 333.1166 |
| | 7-tetrahydro-1H-pyrrolo[3, | | |
| | 2-c]pyridin-2-yl)pyridin- | | |
| | 2-yl]benzonitrile | | |
| 121 | 2-[2-(4-benzoylphenyl)pyridin- | 394.155 | 394.1526 |
| | 4-yl]-1,5,6,7-tetrahydro-4H- | | |
| | pyrrolo[3,2-c]pyridin-4-one | | |
| | trifluoroacetate | | |
| 122 | 2-{2-[4- | 358.155 | 358.1549 |
| | (cyclopropylcarbonyl)phenyl]pyridin- | | |
| | 4-yl}-1,5,6,7-tetrahydro-4H- | | |
| | pyrrolo[3,2-c]pyridin-4-one | | |
| | trifluoroacetate | | |
| 123 | 2-{2-[4- | 408.1707 | 408.1736 |
| | (phenylacetyl)phenyl]pyridin- | | |
| | 4-yl}-1,5,6,7-tetrahydro-4H- | | |
| | pyrrolo[3,2-c]pyridin-4-one | | |
| | trifluoroacetate | | |

### EXAMPLE 124

This illustrates the production of methyl 4-(2-oxo-2-{4-[4-(4-oxo-4,5,6,7-tetrahydro-1H-pyrrolo[3,2-c]pyridin-2-yl)pyridin-2-yl]phenyl}ethoxy)benzoate.

Step 1: methyl 4-{2-oxo-2-[4-(4,4,5,5-tetramethyl-1,3,2-dioxaborolan-2-yl)phenyl]ethoxy}benzoate was prepared using the general procedure described for Example 109.

Step 2: A mixture of 2-(2-chloropyridin-4-yl)-1,5,6,7-tetrahydro-4H-pyrrolo[3,2-c]pyridin-4-one (425.0 g, 1.70 mmol), methyl 4-{2-oxo-2-[4-(4,4,5,5-tetramethyl-1,3,2-dioxaborolan-2-yl)phenyl]ethoxy}benzoate (1.88 mmol) and cesium carbonate, 2.0 M solution (3.0 ml, 6.0 mmol) in DMF (10 ml) was purged with nitrogen for 20 minutes. To this mixture was added tetrokistriphenylhosphinepalladium (185.0 mg, 0.16 mmol) and resultant mixture heated to 80° C. overnight. The mixture was cooled to ambient temperature and filtered through a cake of Celite. Purification was accomplished by reversed phase HPLC yielding 143.0 mg of an orange solid. ¹HNMR (400 MHz, DMSO-d₆) δ 12.01 (s, 1H), 8.59 (d, J=5.2 Hz, 1H), 8.33 (d, J=8.4 Hz, 2H), 8.31 (s, 1H), 8.12 (d, J=8.4 Hz, 2H), 7.88 (d, J=8.8 Hz, 2H), 7.61 (dd, J=3.6, 1.6 Hz, 1H), 7.19 (d, J=2.0 Hz, 1H), 7.08 (d, J=8.8 Hz, 2H), 7.05 (s, 1H), 5.75 (s, 1H), 3.79 (s, 3H), 4.54 (t, J=5.6 Hz, 2H), 2.86 (t, J=6.8 Hz, 2H). m/z (M+H) 482.29.

### EXAMPLE 125

This illustrates the production of 2-{2-[4-(morpholin-4-ylacetyl)phenyl]pyridin-4-yl}-1,5,6,7-tetrahydro-4H-pyrrolo[3,2-c]pyridin-4-one trifluoroacetate.

Step 1: 2-morpholin-4-yl-1-[4-(4,4,5,5-tetramethyl-1,3,2-dioxaborolan-2-yl)phenyl]ethanone was prepared using the general procedure in Example 109.

Step 2: A mixture of 2-(2-chloropyridin-4-yl)-1,5,6,7-tetrahydro-4H-pyrrolo[3,2-c]pyridin-4-one (425.0 mg, 1.70 mmol), 2-morpholin-4-yl-1-[4-(4,4,5,5-tetramethyl-1,3,2-dioxaborolan-2-yl)phenyl]ethanone (1.96 mmol) and cesium carbonate, 2.0 M solution (3.0 ml, 6.0 mmol) in DMF (10 ml) was purged with nitrogen for 20 minutes. To this mixture was added tetrokistriphenylhosphinepalladium (185.0 mg, 0.16 mmol) and resultant mixture heated to 80° C. overnight. The mixture was cooled to ambient temperature and filtered through a cake of Celite. Purification was accomplished by sequestering the solution on MP—OH resin in methanol. After shaking for one hour the product was removed with 2.0 M solution ammonia dissolved in methanol. Tan solids formed. The solids were filtered and washed with methanol yielding 31.7 mg of desired compound. ¹HNMR (400 MHz, CD₃OD) δ 8.58 (d, J=6.0 Hz, 1H), 8.29 (d, J=1.6 Hz, 1H), 8.20 (m, 4H), 7.83 (dd, J=6.0, 2.0 Hz, 1H), 7.38 (s, 1H), 5.06 (s, 2H), 3.99 (br.s, 1H), 3.58 (t, J=7.2 Hz, 2H), 2.98 (t, J=7.2 Hz, 2H), 1.16 (s, 8H). m/z (M+H) 417.29.

### EXAMPLE 126

This illustrates the production of 2-[2-(1,4-benzodioxin-6-yl)pyridin-4-yl]-1,5,6,7-tetrahydro-4H-pyrrolo[3,2-c]pyridin-4-one trifluoroacetate. 2-[2-(1,4-benzodioxin-6-yl)pyridin-4-yl]-1,5,6,7-tetrahydro-4H-pyrrolo[3,2-c]pyridin-4-one trifluoroacetate was prepared from 6-bromo-1,4-benzodioxine (prepared by the method described in J. Org. Chem., 1987, 52, 5619) using the procedure outlined for Example 109. ¹H NMR (400 MHz, MeOD-d₄): δ 8.44, d, J=6.4, 1H; 8.20, d, J=1.8, 1H; 7.89, dd, J=6.8, 1.8, 1H; 7.48, s, 1H; 7.45, dd, J=8.5, 2.3, 1H; 7.26, d, J=2.3, 1H; 6.88, d, J=8.5, 1H; 6.1, s, 2H; 3.59, t, J=6.08, 2H; 3.00, t, J=6.0, 2H. m/z 346 (M+H) Calculated for C₂₀H₁₅N₃O+H: 346.1186. Found: 346.1197.

### EXAMPLE 127

This illustrates the production of 2-[2-(1H-indazol-5-yl)pyridin-4-yl]-1,5,6,7-tetrahydro-4H-pyrrolo[3,2-c]pyridin-4-one. The title compound was prepared from 5-bromoindazole (Organic Reactions Vol. 5,1949, 198-206) by the procedure outlined for Example 109. m+H: 330.

### EXAMPLE 128

This illustrates the production of 2-[2-(2,3-dihydro[1,4]dioxino[2,3-b]pyridin-7-yl)pyridin-4-yl]-1,5,6,7-tetrahydro-4H-pyrrolo[3,2-c]pyridin-4-one bis(trifluoroacetate). 7-Bromo-2,3-dihydro[1,4]dioxino[2,3-b]pyridine (Davies et al. WO02/056882A1 (2002)) was converted to 7-(4,4,5,5-tetramethyl-1,3,2-dioxaborolan-2-yl)-2,3-dihydro[1,4]dioxino[2,3-b]pyridine by the procedure described for Example 109. The title compound was prepared from 2-(2-chloropyridin-4-yl)-1,5,6,7-tetrahydro-4H-pyrrolo[3,2-c]pyridin-4-one and 7-(4,4,5,5-tetramethyl-1,3,2-dioxaborolan-2-yl)-2,3-dihydro[1,4]dioxino[2,3-b]pyridine by the procedure described for Example 2. ¹H NMR (400 MHz, DMSO-d₆) δ 12.17 (s, 1H), 8.58 (d, J=5.8, 1H), 8.51 (d, J=2.2, 1H), 8.30 (s, 1H), 8.00 (d, J=2.2, 1H), 7.75 (d, J=4.8, 1H), 7.41 (s, 1H), 7.15 (s, 1H), 4.51-4.46 (m, 2H), 4.35-4.31 (m, 2H), 3.43 (t, J=6.7, 2H), 2.89 (t, J=6.8, 2H). HRMS calculated for C₁₉H₁₇N₄O₃ (MH⁺) 349.1295, found 349.1291.

### EXAMPLE 129

This illustrates the production of 2-(2-[1,4]dioxino[2,3-b]pyridin-7-ylpyridin-4-yl)-1,5,6,7-tetrahydro-4H-pyrrolo[3,2-c]pyridin-4-one bis(trifluoroacetate).

A mixture of 7-bromo-2,3-dihydro[1,4]dioxino[2,3-b]pyridine (Davies et al. WO02/056882A1 (2002)) (946 mg, 4.38 mmol) in carbon tetrachloride (60 mL) was treated with N-bromosuccinimide (1.7 g, 9.63 mmol), followed by 2,2′-azobisisobutyronitrile (60 mg) The suspension was refluxed for 2 days. The reaction mixture was diluted with dichloromethane and washed with water. The organic layer was filtered and concentrated to give crude 2,3,7-tribromo-2,3-dihydro[1,4]dioxino[2,3-b]pyridine (2.42 g). The residue was dissolved in acetone (50 mL) and treated with sodium iodide (3.3 g, 21.9 mmol). The mixture was refluxed overnight. The reaction mixture was concentrated, suspended in dichloromethane, and washed with 10% sodium thiosulfate. The organic layer was dried (sodium sulfate, concentrated, and purified by flash chromatography (10→70% ethyl acetate/hexanes) to give 7-bromo[1,4]dioxino[2,3-b]pyridine as a white solid (291 mg, 1.36 mmol, 31% yield). LC-MS (ES+) MH⁺=214, 216. ¹H NMR (400 MHz, DMSO-d₆) δ 7.82 (d, J=2.1, 1H), 7.50 (d, J=2.1, 1H), 6.36 (d, J=3.5, 1H), 6.33 (d, J=3.7, 1H).

7-bromo[1,4]dioxino[2,3-b]pyridine was converted to 7-(4,4,5,5-tetramethyl-1,3,2-dioxaborolan-2-yl)[1,4]dioxino[2,3-b]pyridine by the procedure described for Example 109. 2-(2-[1,4]dioxino[2,3-b]pyridin-7-ylpyridin-4-yl)-1,5,6,7-tetrahydro-4H-pyrrolo[3,2-c]pyridin-4-one bis(trifluoroacetate) was prepared from 2-(2-chloropyridin-4-yl)-1,5,6,7-tetrahydro-4H-pyrrolo[3,2-c]pyridin-4-one (Example 1) and 7-(4,4,5,5-tetramethyl-1,3,2-dioxaborolan-2-yl)[1,4]dioxino[2,3-b]pyridine by the procedure described for Example 2. ¹H NMR (400 MHz, DMSO-d₆) δ 12.1 (s, 1H), 8.58 (d, J=5.7, 1H), 8.47 (d, J=2.1, 1H), 8.26 (d, J=1.2, 1H), 7.84 (d, J=2.0, 1H), 7.71 (dd, J=5.6,1.3, 1H), 7.37 (d, J=2.1, 1H), 7.13 (s, 1H), 6.40 (A of AB, J=3.5, 1H), 6.38 (B of AB, J=3.5, 1H), 3.42 (t, J=6.7, 2H), 2.88 (t, J=6.8, 2H). HRMS calculated for C₁₉H₁₅N₄O₃ (MH⁺) 347.1139, found 347.1123.

### EXAMPLE 130

This example illustrates the production of 2-{2-[3-(bromomethyl)phenyl]pyridin-4-yl}-1,5,6,7-tetrahydro-4H-pyrrolo[3,2-c]pyridin-4-one. (Angew. Chem. Int. Ed. Engl. 1980,19,394). 2-{2-[4-(hydroxymethyl)phenyl]pyridin-4-yl}-1,5,6,7-tetrahydro-4H-pyrrolo[3,2-c]pyridin-4-one (350 mg, 1.1 mmol) was suspended in 5 ml of 30% HBr in acetic acid, and kept stirred overnight. The reaction mixture was then concentrated and the residue was triturated with ethyl acetate. The title compound was collected by filtration as yellow solid (387 mg). ¹HNMR (400 MHz, DMSO-d₆): δ 12.8 (s, 1H), 8.72 (d, 1H), 8.55 (ds, 1H), 8.19 (s, 1H), 8.16 (dd, 1H), 8.03 (d, 1H), 7.79 (d, 1H), 7.74 (ds, 1H), 7.69 (t, 1H), 4.83(s, 2H), 3.46 (t, 2H), 2.96 (t, 2H); m/z: 382.1(M+H).

### EXAMPLE 131

This example illustrates the production of 5-[4-(4-oxo-4,5,6,7-tetrahydro-1H-pyrrolo[3,2-c]pyridin-2-yl)pyridin-2-yl]-1-benzofuran-2-carboxylic acid hydrochloride. Ethyl 5-bromo-1-benzofuran-2-carboxylate (Bioorg. Med. Chem. 5:445 (1997)) was converted to ethyl 5-(4,4,5,5-tetramethyl-1,3,2-dioxaborolan-2-yl)-1-benzofuran-2-carboxylate by the procedure described for Example 109. A mixture of 2-(2-chloropyridin-4-yl)-1,5,6,7-tetrahydro-4H-pyrrolo[3,2-c]pyridin-4-one (Example 1) (1.8 g, 7.2 mmol), ethyl 5-(4,4,5,5-tetramethyl-1,3,2-dioxaborolan-2-yl)-1-benzofuran-2-carboxylate (3.4 g, 10.8 mmol), tetrakis(triphenylphospine)palladium(0) (416 mg, 0.36 mmol), 2.0 M aqueous sodium carbonate (10.8 mL, 21.6 mmol), and dimethylformamide (40 mL) was stirred at 115° C. under nitrogen for 16 hours. The reaction mixture was cooled, diluted with water, and treated with 10 mL of 10% NaOH. The aqueous layer was washed with ethyl acetate and filtered. The pH of the filtrate was adjusted to 5 with aq. HCl. The resultant precipitate was filtered, washed with water and diethyl ether, and dried to give 5-[4-(4-oxo-4,5,6,7-tetrahydro-1H-pyrrolo[3,2-c]pyridin-2-yl)pyridin-2-yl]-1-benzofuran-2-carboxylic acid hydrochloride as an orange solid (2.35 g, 5.73 mmol, 80% yield). ¹H NMR (300 MHz, DMSO-d₆) δ 12.10 (s, 1H), 8.57 (s, 2H), 8.30 (s, 2H), 7.84 (d, J=8.7, 1H), 7.76 (s, 1H), 7.62 (d, J=4.4, 1H), 7.22 (s, 1H), 7.08 (s, 1H), 3.50-3.37 (m, 2H), 2.88 (t, J=5.8, 2H). HRMS calculated for C₂₁H₁₆N₃O₄ (MH⁺) 374.1135, found 374.1145.

### EXAMPLE 132

This example illustrates the production of 6-[4-(4-oxo-4,5,6,7-tetrahydro-1H-pyrrolo[3,2-c]pyridin-2-yl)pyridin-2-yl]-1H-indole-2-carboxylic acid hydrochloride. 6-Bromo-2-carboxyindole ethyl ester was converted to ethyl 6-(4,4,5,5-tetramethyl-1,3,2-dioxaborolan-2-yl)-1H-indole-2-carboxylate by the procedure described for Example 109. A mixture of 2-(2-chloropyridin-4-yl)-1,5,6,7-tetrahydro-4H-pyrrolo[3,2-c]pyridin-4-one (Example 1) (1.8 g, 7.2 mmol), ethyl 6-(4,4,5,5-tetramethyl-1,3,2-dioxaborolan-2-yl)-1H-indole-2-carboxylate (3.4 g, 10.8 mmol), tetrakis(triphenylphospine)palladium(0) (416 mg, 0.36 mmol), 2.0 M aqueous sodium carbonate (10.8 mL, 21.6 mmol), and dimethylformamide (40 mL) was stirred at 115° C. under nitrogen for 16 hours. 1.0 N LiOH (10 mL), 2.0 M cesium carbonate (10 mL), and methanol (10 mL) was added, and the resultant mixture was heated at 80° C. for six hours. The reaction mixture was cooled, diluted with water, and treated with 10 mL of 10% NaOH. The aqueous layer was washed with ethyl acetate and filtered. The pH of the filtrate was adjusted to 5 with aq. HCl. The resultant precipitate was filtered, washed with water and diethyl ether, and dried to give 5-[4-(4-oxo-4,5,6,7-tetrahydro-1H-pyrrolo[3,2-c]pyridin-2-yl)pyridin-2-yl]-1-benzofuran-2-carboxylic acid hydrochloride as an orange solid (2.35 g, 5.73 mmol, 80% yield). ¹H NMR (300 MHz, DMSO-d₆) δ 12.10 (s, 1H), 11.85 (s, 1H), 8.55 (d, J=5.2, 1H), 8.27 (s, 1H), 8.20 (s, 1H), 7.86 (dd, J=8.5, 1.3, 1H), 7.73 (d, J=8.6, 1H), 7.54 (dd, J=5.3,1.4, 1H), 7.12 (d, J=2.2, 1H), 7.06 (s, 1H), 3.42 (td, J=6.6, 2.2, 2H), 2.88 (t, J=6.7, 2H). HRMS calculated for C₂₁H₁₇N₄O₃ (MH⁺) 373.1295, found 373.1316.

### EXAMPLE 133

This example illustrates the production of 2-{2-[4-(N-tert-butoxycarbonyl-aminoacetyl)phenyl]pyridin-4-yl}-1,5,6,7-tetrahydro-4H-pyrrolo[3,2-c]pyridin-4-one.

Step 1. Preparation of 2-(N-tert-butoxycarbonylamino)-1-(4-bromophenyl)ethanone. A suspension of 2-amino-1-(4-bromophenyl)ethanone (1.00 g, 3.99 mmol) in 9:1 THF/water (30 mL) was treated with sodium bicarbonate (1.34 g, 16.0 mmol) followed by a 1.0 M solution of di-tert-butyl dicarbonate in THF (4.4 mL, 4.4 mmol). After stirring for 2 hours, the reaction mixture was partitioned between water and ethyl acetate. The organic layer was washed with brine, dried (sodium sulfate), and concentrated to give 2-(N-tert-butoxycarbonylamino)-1-(4-bromophenyl)ethanone as an off-white solid (1.20 g, 3.82 mmol, 96% yield). ¹H NMR (300 MHz, acetone-d₆) δ 7.98 (d, J=8.6, 2H), 7.76 (d, J=8.7, 2H), 6.18 (bs, 1H), 4.60 (d, J=5.6, 2H), 1.44 (s, 9H).

Step 2. Preparation of 2-{2-[4-(N-tert-butoxycarbonyl-aminoacetyl)phenyl]pyridin-4-yl}-1,5,6,7-tetrahydro-4H-pyrrolo[3,2-c]pyridin-4-one. 2-(N-tert-butoxycarbonylamino)-1-(4-bromophenyl)ethanone was converted to 2-(N-tert-butoxycarbonylamino)-1-[4-(4,4,5,5-tetramethyl-1,3,2-dioxaborolan-2-yl)phenyl]ethanone by the procedure described for Example 109. A mixture of 2-(2-chloropyridin-4-yl)-1,5,6,7-tetrahydro-4H-pyrrolo[3,2-c]pyridin-4-one (Example 1) (627 mg, 2.53 mmol), 2-(N-tert-butoxycarbonylamino)-1-[4-(4,4,5,5-tetramethyl-1,3,2-dioxaborolan-2-yl)phenyl]ethanone (1.61 g, 3.8 mmol), tetrakis(triphenylphospine)palladium(0) (146 mg, 0.127 mmol), 2.0 M aqueous cesium carbonate (3.8 mL, 7.6 mmol), and dimethylformamide (12 mL) was stirred at 80° C. under nitrogen for several days. The reaction mixture was partitioned between water and ethyl acetate. The organic layers were washed with brine, dried (sodium sulfate), concentrated, and purified by flash chromatography (0→20% methanol/ethyl acetate) to give 2-{2-[4-(N-tert-butoxycarbonyl-aminoacetyl)phenyl]pyridin-4-yl}-1,5,6,7-tetrahydro-4H-pyrrolo[3,2-c]pyridin-4-one as a yellow solid (545 mg, 1.22 mmol, 48% yield). ¹H NMR (300 MHz, DMSO-d₆) δ 12.02 (s, 1H), 8.60 (d, J=5.2, 1H), 8.38-8.28 (m, 3H), 8.10 (d, J=8.5, 2H), 7.64 (d, J=5.2, 1H), 7.21 (s, 1H), 7.15-7.02 (m, 2H), 4.50 (d, J=5.6, 2H), 3.42 (td, J=6.5, 1.8, 2H), 2.88 (t, J=6.7, 2H), 1.40 (s, 9H). HRMS calculated for C₂₅H₂₇N₄O₄ (MH⁺) 447.2027, found 447.2039.

### EXAMPLE 134

This example illustrates the production of 2-{2-[3-(morpholin-4-ylacetyl)phenyl]pyridin-4-yl}-1,5,6,7-tetrahydro-4H-pyrrolo[3,2-c]pyridin-4-one bis(trifluoroacetate).

Step 1: 1-(3-bromophenyl)-2-morpholin-4-ylethanone: A solution of 2-bromo-1-(3-bromophenyl)ethanone (2.78 g, 10 mmol) in dichloromethane (50 mL) was added to a solution of morpholine (87 mL, 100 equiv.) in dichloromethane (300 mL). After 2 hours the solvents were removed the residue dissolved in dichloromethane and washed with water (×5), and extracted with 3M hydrochloric acid. The pH of the aqueous extracts was adjusted to 8 with sodium hydroxide and the resulting precipitate collected. The title compound was obtained as an off-white solid (2.35 g, 83%) ¹H NMR (400 MHz, CDCl₃): δ 8.09, t, J=1.6, 1H; 7.89, d, J=7.7, 1H; 7.65, d, J=8, 1H; 7.30, t, J=7.8, 1H; 3.74-3.72, m, 6H; 2.56, t, J=4.6, 4H. m/z 284 (M+H) Calculated for C₁₂H₁₄NO₂Br+H: 284.0281. Found: 284.0294.

Step 2: 2-{2-[3-(morpholin-4-ylacetyl)phenyl]pyridin-4-yl}-1,5,6,7-tetrahydro-4H-pyrrolo[3,2-c]pyridin-4-one bis(trifluoroacetate was prepared from 1-(3-bromophenyl)-2-morpholin-4-ylethanone using the procedure outlined for Example 109. Yield: 80%. ¹H NMR (400 MHz, MeOD-d₄): δ 8.61-8.59, m, 2H; 8.36, d, J=1.6, 1H; 8.31, d, J=7.9, 1H; 8.28, d, J=7.9, 1H; 7.93, dd, J=7.9, 4.4, 1H; 7.85, t, J=7.9, 1H; 7.46, s, 1H; 5.12, s, 2H; 4.02, bs, 4H; 3.59, t, J=6.9, 2H; 3.29, bs, 4H; 3.00, t, J=6.9, 2H . m/z 417 (M+H) Calculated for C₂₄H₂₄N₄O₃+H: 417.1921. Found: 417.1923.

### EXAMPLE 135

This example illustrates the production of 2-{2-[3-(aminoacetyl)phenyl]pyridin-4-yl}-1,5,6,7-tetrahydro-4H-pyrrolo[3,2-c]pyridin-4-one bis(trifluoroacetate).

Step 1: N-Boc-2-amino-1-(3-bromophenyl)ethanone: A suspension of hexamethylene tetramine (2.01 g, 14.3 mmol) in dichloromethane (5 mL) was added to a solution of 2-bromo-1-(3-bromophenyl)ethanone (3.89 g, 14 mmol) in dichloromethane (25 mL). The thick heterogeneous suspension was diluted with 20 mL dichloromethane and the solids isolated by filtration. The solid was suspended in ethanol (50 mL) and treated with concentrated aqueous hydrochloric acid (4.5 mL). After 16 hours, the solids were isolated by filtration and treated with saturated aqueous bicarbonate and extracted with dichloromethane. The organic extracts were treated with Boc-anhydride (1M solution in tetrahydrofuran, 15 mL) and stirred for 16 hours. The solution was diluted with dichloromethane, washed with saturated aqueous ammonium chloride, dried over sodium sulfate filtered and evaporated to give an orange oil. The product was purified by silica gel chromatography. Collected 2.37 g (58%) of the title compound as a light yellow oil. ¹H NMR (400 MHz, CDCl₃): δ 8.06, s, 1 H: 7.85, dd, J=7.7, 1.0, 1H; 7.70, dd, J=7.8, 10, 1H; 7.34, t, J=7.8, 1H; 5.44, bs, 1H; 4.58, d, J=4.5, 2H; 1.44, s, 9H. m/z 314 (M+H) Calculated for C₁₃H₁₆NO₃Br+H: 314.0386. Found: 314.0370.

Step 2: 2-{2-[3-(aminoacetyl)phenyl]pyridin-4-yl}-1,5,6,7-tetrahydro-4H-pyrrolo[3,2-c]pyridin-4-one bis(trifluoroacetate): was prepared from N-Boc-2-amino-1-(3-bromophenyl)ethanone using the procedure outlined for Example 109. Yield: 43%. ¹H NMR (400 MHz, MeOD-d₄): δ 8.60-8.59, m, 2H; 8.41, d, J=1.6, 1H; 8.30, d, J=7.9, 1H; 8.25, d, J=7.9, 1H; 8.01, dd, J=6.7,1.9, 1H; 7.89, t, J=7.9, 1H; 7.54, s, 1H; 4.71, s, 2H; 3.58, t, J=7.0, 2H; 3.00, t, J=7.0, 2H. m/z 347 (M+H) Calc for C₂₀H₁₈N₄O₂+H: 347.1503. Found: 347.1518.

### EXAMPLE 136

This example illustrates the production of 2-[2-(3-acetyl-5-chlorophenyl)pyridin-4-yI]-1,5,6,7-tetrahydro-4H-pyrrolo[3,2-c]pyridin-4-one trifluoroacetate.

Step 1: 3-bromo-5-chloroacetopheone: 1,3-dibromo-5-chlorobenzene (811 mg, 3 mmol) was dissolved in ethyl ether (20 mL) and cooled to −78° C. in a dry-ice/acetone bath. N-butyllithium (1.6 M solution in hexanes, 1.1 equiv, 1.9 mL) was added dropwise. After stirring for 2 hours, dimethylformamide (2 mL) was added and the solution warmed to room temperature. The reaction was quenched with saturated aqueous ammonium chloride and diluted with ethyl acetate. Washed with water (×2), dried over sodium sulfate, filtered and evaporated. The residue was purified by silica gel chromatography to yield 150 mg (21%) of the title compound as an orange oil. ¹H NMR (400 MHz, CDCl₃): δ 7.92, t, J=1.6, 1H; 7.81, t, J=1.6, 1H; 7.67, t, J=1.6, 1H; 2.55, s, 3H.

Step 2: 2-[2-(3-acetyl-5-chlorophenyl)pyridin-4-yl]-1,5,6,7-tetrahydro-4H-pyrrolo[3,2-c]pyridin-4-one trifluoroacetate was prepared from 3-bromo-5-chloroacetopheone using the procedure outlined for Example 109. Yield: 25%. ¹H NMR (400 MHz, DMSO-d₆): δ 12.1, s, 1H; 8.60, d, J=5.5, 1H; 8.57, s, 1H; 8.41, s, 1H; 8.34, s, 1H; 8.03, s, 1H; 7.70, d, J=5.5, 1H; 7.33, s, 1H; 7.08, s, 1H; 3.39, t, J=6.5 2H; 2.85, t, J=6.5, 2H; 2.65, s, 3H. m/z 366 (M+H) Calculated for C₂₀H₁₆ClN₃O₂+H: 366.1004. Found: 366.1007.

### EXAMPLE 137

This example illustrates the production of 2-[2-(3-acetyl-5-fluorophenyl)pyridin-4-yl]-1,5,6,7-tetrahydro-4H-pyrrolo[3,2-c]pyridin-4-one trifluoroacetate. The title compound was prepared from 1,3-dibromo-5-fluorobenzene by the procedure outlined for Example 136. ¹H NMR (400 MHz, DMSO-d₆): δ 12.03, s, 1H; 8.58, d, J=5.3, 1H; 8.51, s, 1H; 8.32, s, 1H; 8.22, d, 9.2, 1H; 7.80, d, J=9.0, 1H; 7.65, d, J=5.3; 7.28, s, 1H; 1.05, s, 1H; 2.85, t, J=6.8, 2H; 2.65, s, 3H. m/z 350 (M+H) Calculated for C₂₀H₁₆FN₃O₂+H: 350.1299. Found: 350.1285.

### EXAMPLE 138

This example illustrates the production of 2-[2-(5-phenylthien-2-yl)pyridin-4-yl]-1,5,6,7-tetrahydro-4H-pyrrolo[3,2-c]pyridin-4-one.

Step 1: 4,4,5,5-tetramethyl-2-(5-phenylthiophene-2-yl)-1,3,2-dioxaborolane 2.5M BuLi in hexanes (0.45 mL, 1.2 mmol) was added slowly to a dry-ice/acetone cooled solution of 2-iodo-5-phenyl thiophene (286 mg, 1 mmol) in dry THF (2 mL). The resulting mixture was stirred at −78° C. for 5 minutes, and then isopropyl pinacol borate (0.25 mL, 1.2 mmol) was added. The mixture was slowly warmed to room temperature, and the green solution was diluted with EtOAc, washed with 1 M HCl, water and then brine. The organic extract was dried over sodium sulfate, and concentrated to give 300 mg of 4,4,5,5-tetramethyl-2-(5-phenylthiophene-2-yl)-1,3,2-dioxaborolane as a blue oil. Calculated exact mass 287.1277 (M+H⁺); Found positive electrospray LC-MS, m/e 287 (M+H⁺).

Step 2: 2-[2-(5-phenylthien-2-yl)pyridin-4-yl]-1,5,6,7-tetrahydro-4H-pyrrolo[3,2-c]pyridin-4-one was prepared by the method described for Example 2. m/e 372 (M+H⁺).

### EXAMPLE 139

This example illustrates the production of ethyl 3′-[4-(4-oxo-4,5,6,7-tetrahydro-1H-pyrrolo[3,2-c]pyridin-2-yl)pyridin-2-yl]-1,1′-biphenyl-3-carboxylate.

Step 1: Standard Suzuki coupling at 80° C. overnight and purification by reverse phase HPLC gave ethyl 3′-bromo-1,1′-biphenyl-3-carboxylate as a colorless oil. Calculated exact mass 305.0177 (M+H⁺); Found positive electrospray LC-MS, m/e 305 (M+H⁺).

Step 2: A mixture of ethyl 3′-bromo-1,1′-biphenyl-3-carboxylate (3.2 g, 10.5 mmol), PdCl₂(dppf) (230 mg, 0.3 mmol), KOAc (3.0 g, 30.6 mmol), and bis(pinacolato)diboron (2.7 g, 10.6 mmol) in DMF (50 mL) was heated to 80° C. for 10 hrs, then cooled to room temperature. The reaction mixture was the filtered through a syringe filter (0.45 um), purified by flash column chromatograph to give 2.9 g of ethyl 3′-(4,4,5,5-tetramethyl-1,3,2-dioxaborolan-2-yl)-1,1′-biphenyl-3-carboxylate as an off-white solid. Calculated exact mass 353.1924 (M+H⁺); Found positive electrospray LC-MS, m/e 353 (M+H⁺).

Step 3: ethyl 3′-[4-(4-oxo-4,5,6,7-tetrahydro-1H-pyrrolo[3,2-c]pyridin-2-yl)pyridin-2-yl]-1,1′-biphenyl-3-carboxylate was prepared by the method described for Example 2. m/e 438 (M+H⁺).

The following compounds were prepared in a similar manner. Carboxylic acids were prepared by hydrolysis of the corresponding esters.

| Example | | Calculated | Found |
|---|---|---|---|
| No. | Compound Name(s) | (m + H) | (m + H) |
| 140 | 3′-[4-(4-oxo-4,5,6,7-tetrahydro- | 410.1505 | 410 |
| | 1H-pyrrolo[3,2-c]pyridin- | | |
| | 2-yl)pyridin-2-yl]-1,1′ biphenyl- | | |
| | 3-carboxylic acid | | |
| 141 | ethyl 3′-[4-(4-oxo-4,5,6, | 438.1818 | 438 |
| | 7-tetrahydro-1H-pyrrolo[3, | | |
| | 2-c]pyridin-2-yl)pyridin-2-yl]-1, | | |
| | 1′-biphenyl-4-carboxylate | | |
| 142 | 3′-[4-(4-oxo-4,5,6,7-tetrahydro- | 410.1505 | 410 |
| | 1H-pyrrolo[3,2-c]pyridin- | | |
| | 2-yl)pyridin-2-yl]-1,1′- | | |
| | biphenyl-4-carboxylic acid | | |

### EXAMPLE 143

This example illustrates the production of 2-{2-[3′-(morpholin-4-ylcarbonyl)-1,1′-biphenyl-3-yl]pyridin-4-yl}-1,5,6,7-tetrahydro-4H-pyrrolo[3,2-c]pyridin-4-one.

A 0.2M mixture of carboxylic acid (1 equivalent), HOBT (1.2 equivalents), EDC (1.2 equivalents) and DIEA (3 equivalents) in DMF was stirred at rt for 1 h, then morpholine (1.0 equivalent) was added. The cloudy mixture was stirred at rt overnight, and purified by reverse-phase HPLC to give morpholine amide, which were characterized by analytical reverse phase HPLC, NMR, and MS. The following compounds were prepared with this method.

| Example | | Calculated | Found |
|---|---|---|---|
| No. | Compound Name(s) | (m + H) | (m + H) |
| 143 | 2-{2-[3′-(morpholin-4-ylcarbonyl)- | 479.2083 | 479 |
| | 1,1′-biphenyl-3-yl]pyridin-4-yl}- | | |
| | 1,5,6,7-tetrahydro-4H-pyrrolo[3, | | |
| | 2-c]pyridin-4-one | | |
| 144 | 2-{2-[4′-(morpholin-4-ylcarbonyl)- | 479.2083 | 479 |
| | 1,1′-biphenyl-3-yl]pyridin-4-yl}- | | |
| | 1,5,6,7-tetrahydro-4H-pyrrolo[3, | | |
| | 2-c]pyridin-4-one | | |

### EXAMPLE 145

This example illustrates the production of 5-[4-(4-oxo-4,5,6,7-tetrahydro-1H-pyrrolo[3,2c]pyridin-2-yl)pyridin-2-yl]-2-furaldehyde trifluoroacetate. A solution of furfuraldehyde, diethylacetal (4.89 g, 28.7 mmol) in dimethoxyethane (55 mL) was cooled to −20° C. under nitrogen. A solution of 2.5M n-butyl lithium (13.8 mL, 34.4 mmol) was added slowly. After two hours at −20° C. isopropyl borate (7.95 mL, 34.4 mmol) was added. After warming to 20° C. over a two-hour period acetic acid (2.17 mL, 37 mmol) and water (2.6 mL) were added. To the above solution was added 2-(2-chloropyridin-4-yl)-1,5,6,7-tetrahydro-4H-pyrrolo[3,2-c]pyridin-4-one (3.00 g, 11.5 mmol) ethanol (37 mL) triethylamine (3.2 mL, 22.9 mmol) and 1,1′-bis(diphenylphosphino)ferocene palladium (II) chloride, 1:1 complex with methylene chloride (1.75 g, 2.38 mmol). The mixture was flushed with nitrogen and stirred at 60° C. for11 hours. The mixture was filtered, poured into water (500 mL) and extracted with ethyl acetate. The extract was concentrated and the residue was purified by reverse phase chromatography to give 5-[4-(4-oxo-4,5,6,7-tetrahydro-1H-pyrrolo[3,2c]pyridin-2-yl)pyridin-2-yl]-2-furaldehyde as a yellow solid (2.36 g). ¹H NMR (d6-DMSO): δ 12.20 (s, 1H), 9.67 (s, 1H), 8.53 (d, J=5.2 Hz, 1H), 8.18 (d, J=1.2 Hz, 1H), 7.66-7.69 (m, 2H), 7.41 (d, J=3.7 Hz, 1H), 7.08 (bs, 1H), 3.40 (m, J=2H), 2.85 (t, J=7.0 Hz, 2H). High resolution MS calculated for C₁₇H₁₄N₃O₃ (M+H⁺)=308.1030. Found 308.1039.

### EXAMPLE 146

This example illustrates the production of 2-{2-[5-(hydroxymethyl)-2-furyl]pyridin-4-yl}-1,5,6,7-tetrahydro-4H-pyrrolo[3,2-c]pyridin-4-one trifluoroacetate. To a solution of 5-[4-(4-oxo-4,5,6,7-tetrahydro-1H-pyrrolo[3,2c]pyridin-2-yl)pyridin-2-yl]-2-furaldehyde trifluoroacetate (0.42 g, 1.0 mmol) in ethanol (15 mL) and water (2 mL) was added sodium borohydride (10 mg) followed by sodium cyanoborohydride (3×20 mg). After stirring overnight the mixture was concentrated, and the residue was dissolved in water (20 mL) and trifluoroacetic acid (0.5 mL). The solution was purified by reverse phase chromatography to give 2-{2-[5-(hydroxymethyl)-2-furyl]pyridin-4-yl}-1,5,6,7-tetrahydro-4H-pyrrolo[3,2-c]pyridin-4-one trifluoroacetate as a yellow solid (140 mg). ¹H NMR (D₂O): δ 7.85 (d, J=6.4 Hz, 1H), 7.19 (d, J=1.2 Hz, 1H), 7.03 (dd, J=6.4 Hz, 1.2 Hz, 1H), 6.88 (d, J=3.6 Hz, 1H), 6.56 (s, 1H), 6.39 (d, J=3.6 Hz, 1H), 4.45 (s, 2H), 3.21 (t, J=7.2 Hz, 2H), 2.48 (t, J=7.0 Hz, 2H). High resolution MS calculated for C₁₇H₁₆N₃O₃ (M+H⁺)=310.1186. Found 310.1174.

### EXAMPLE 147

This example illustrates the production of 2-{2-[5-(hydroxymethyl)thien-3-yl]pyridin-4-yl}-1,5,6,7-tetrahydro-4H-pyrrolo[3,2-c]pyridin-4-one trifluoroacetate. To an ice-bath cooled solution of 4-bromo-2-thiophenecarboxaldehyde (11.46 g, 60.0 mmol) in ethanol (5mmol) was added sodium borohydride (0.70 g, 18.5 mmol). After one hour, acetic acid (1 mL) was added and the mixture was concentrated to dryness. The residue was dissolved in diethyl ether (70 mL) filtered, washed with aqueous sodium bicarbonate and brine, stirred over magnesium sulfate, filtered, and concentrated to give (4-bromothien-2-yl)methanol (11.0 g). The (4-bromothien-2-yl)methanol was converted to 4-(4,4,5,5-tetramethyl-1,3,2-dioxaborolan-2-yl)thien-2-yl]methanol using the pinacoldiborane/Pd(dppf) reaction. This borane was coupled with 2-(2-chloropyridin-4-yl)-1,5,6,7-tetrahydro-4H-pyrrolo[3,2-c]pyridin-4-one to give 2-{2-[5-(hydroxymethyl)thien-3-yl]pyridin-4-yl}-1,5,6,7-tetrahydro-4H-pyrrolo[3,2-c]pyridin-4-one trifluoroacetate as a yellow solid (0.243 g). High resolution MS calculated for C₁₇H₁₆N₃O₂S₁ (M+H⁺) ═326.0958. Found 326.0928. ¹H NMR (d6-DMSO): δ 12.34 (s, 1H), 8.43 (d, J=6.4 Hz, 1H), 8.24 (m, 2H), 7.73 (dd, J=5.2,1.6 Hz, 1H), 7.63 (m, 1H), 7.43 (d, J=6.0 Hz. 1H), 7.18 (bs, 1H), 4.65 (m, 2H), 2.83 (t, J=6.8 Hz, 2H).

### EXAMPLE 148

This example illustrates the production of 2-{2-[6-(hydroxymethyl)-2-naphthyl]pyridin-4-yl}-1,5,6,7-tetrahydro-4H-pyrrolo[3,2-c]pyridin-4-one trifluoroacetate.

Step 1. (Preparation of (6-bromo-2-naphthyl)methanol): A suspension of methyl 6-bromo-2-naphthoate (2.0 g, 7.5 mmol) was cooled to −78° C. and treated with a 1.0 M solution of diisobutylaluminum hydride in tetrahydrofuran (37.6 mL, 37.6 mmol) the reaction was allowed to warm to room temperature and stir for 1 hour. Then cooled to 0° C. and added 10.0 mL MeOH followed by 20.0 mL 1 N HCl and allowed to warm to room temperature. The reaction contents were then poured into 300.0 mL water and extracted three times with ethyl acetate, washed with brine, dried over magnesium sulfate, filtered and condensed to give (6-bromo-2-naphthyl)methanol as an off white solid (1.6 g, 6.7 mmol, 90%). ¹H NMR (300 MHz, DMSO-d₆) δ 8.16 (s, 1H), 7.91-7.83 (m, 3H), 7.60 (d, J=10.6 Hz, 1H), 7.51 (d, J=9.26 Hz, 1H), 5.36 (t, J=5.84 Hz, 1H), 4.46 (d, J=5.4 Hz, 2H). m/z (M+H): 219.

Step 2. (Preparation of [6-(4,4,5,5-tetramethyl-1,3,2-dioxaborolan-2-yl)-2-naphthyl]methanol): The title compound was prepared according to the method described for Example 109 from (6-bromo-2-naphthyl)methanol (500 mg, 2.1 mmol) to give an off-white solid (575 mg, 2.0 mmol, 96%).

Step 3. (Preparation of 2-{2-[6-(hydroxymethyl)-2-naphthyl]pyridin-4-yl}-1,5,6,7-tetrahydro-4H-pyrrolo[3,2-c]pyridin-4-one trifluoroacetate): The title compound was prepared according to the method described for Example 2 from [6-(4,4,5,5-tetramethyl-1,3,2-dioxaborolan-2-yl)-2-naphthyl]methanol (575 mg, 2.0 mmol) and 2-(2-chloropyridin-4-yl)-1,5,6,7-tetrahydro-4H-pyrrolo[3,2-c]pyridin-4-one (250 mg, 1.0 mmol) to give a yellow solid (135 mg, 0.27 mmol, 27%).

### EXAMPLE 149

This example illustrates the production of 6-[4-(4-oxo-4,5,6,7-tetrahydro-1H-pyrrolo[3,2-c]pyridin-2-yl)pyridin-2-yl]-3,4-dihydroisoquinolin-1(2H)-one trifluoroacetate.

Step 1. (Preparation of 6-bromo-3,4-dihydroisoquinolin-1(2H)-one). The title compound was prepared from 5-bromoindan-1-one according to J. Chem. Soc. (C) 1969, 183-188.

Step 2. (Preparation of 6-(4,4,5,5-tetramethyl-1,3,2-dioxaborolan-2-yl)-3,4-dihydroisoquinolin-1(2H)-one): The title compound was prepared according to the method described for Example 109 from 6-bromo-3,4-dihydroisoquinolin-1(2H)-one (1.0 g, 4.4 mmol) to give an off-white solid (150 mg, 0.54 mmol, 12%)

Step3. (Preparation of 6-[4-(4-oxo-4,5,6,7-tetrahydro-1H-pyrrolo[3,2-c]pyridin-2-yl)pyridin-2-yl]-3,4-dihydroisoquinolin-1(2H)-one trifluoroacetate): The title compound was prepared according to the method described for Example 2 from 6-(4,4,5,5-tetramethyl-1,3,2-dioxaborolan-2-yl)-3,4-dihydroisoquinolin-1(2H)-one (140 mg, 0.73 mmol) and 2-(2-chloropyridin-4-yl)-1,5,6,7-tetrahydro-4H-pyrrolo[3,2-c]pyridin-4-one (150 mg, 0.60 mmol) to give a yellow solid (56 mg, 0.15 mmol, 26%). ¹H NMR (300 MHz, DMSO-d₆) δ 12.25 (s, 1H), 8.63 (d, J=5.8 Hz, 1H), 8.37 (s, 1H), 8.10-7.97 (m, 4H), 7.80 (d, J=4.4 Hz, 1H), 7.42 (s, 1H), 7.16 (s, 1H), 3.43 (m, 4H), 3.00 (t, J=6.5 Hz, 2H), 2.90 (t, J=6.8 Hz, 2H). HRMS calculated for C₂₁H₁₈N₄O₂ (MH⁺) 359.1503, found 359.1473. Anal. calculated for C₂₁H₁₈N₄O₂.1.0 TFA.2.1 H₂O C, 54.14; H, 4.58; N, 10.98. Found: C, 54.10; H, 4.34; N, 10.83.

### EXAMPLE 150

This example illustrates the production of 2-(2-{3-[(methylthio)methyl]phenyl}pyridin-4-yl)-1,5,6,7-tetrahydro-4H-pyrrolo[3,2-c]pyridin-4-one trifluoroacetate.

A solution of 2-{2-[3-(bromomethyl)phenyl]pyridin-4-yl}-1,5,6,7-tetrahydro-4H-pyrrolo[3,2-c]pyridin-4-one (Example 130) (250 mg, 0.54 mmol) in 5.0 mL dimethylformamide was treated with sodium thiomethoxide (20 mg, 0.27 mmol) and heated to 60° C. for 3 hours. The reaction was cooled to room temperature, stirred for 16 hours. Then acidified with trifluoroacetic acid, filtered through a syringe filter, purified by rpHPLC, and lyophilized to give the title compound as a yellow solid (180 mg, 0.39 mmol, 70%). ¹H NMR (400 MHz, DMSO-d₆) δ 12.31 (s, 1H), 8.60 (d, J=5.9 Hz, 1H), 8.32 (s, 1H), 8.00 (s, 1H), 7.95 (d, J=7.5 Hz, 1H), 7.83 (d, J=5.9 Hz, 1H), 7.50 (m, 3H), 7.17 (s, 1H), 3.78 (s, 2H), 3.41 (t, J=6.8 Hz, 2H), 2.89 (t, J=6.7 Hz, 3H), 1.98 (s, 3H). HRMS calculated for C₂₀H₁₉N₃OS (MH⁺) 350.1322 found 350.1332. Anal. calculated for C₂₀H₁₉N₃OS.1.3 TFA.1.7 H₂O C, 51.36; H, 4.51; N, 7.95. Found: C, 51.37; H, 4.52; N, 7.95.

### EXAMPLE 151

This example illustrates the production of N-cyclohexyl-2-hydroxy-4-[4-(4-oxo-4,5,6,7-tetrahydro-1H-pyrrolo[3,2-c]pyridin-2-yl)pyridin-2-yl]benzamide trifluoroacetate.

Step 1. (Preparation of 2-hydroxy-4-iodobenzoic acid): The title compound was prepared according to J. Med. Chem. 1997, 40(16) from 4-aminosalicylic acid (1.5 g, 9.8 mmol) to give a tan solid (1.9 g, 7.2 mmol, 73%)

Step 2. (Preparation of N-cyclohexyl-2-hydroxy-4-iodobenzamide): To a solution of 2-hydroxy-4-iodobenzoic acid (1.0 g, 3.79 mmol), EDCI, and 1-hydroxybenzotriazole in 20 mL of methylene chloride was added diisopropylethyl amine (1.0 mL, 6.4 mmol) followed by cyclohexylamine (0.56 mL, 4.92 mmol) and the reaction stirred for 16 hours. Water was added and the reaction was extracted 3 times with methylene chloride, washed with brine, dried over magnesium sulfate and concentrated. The material was purified by flash column chromatography using 5% ethyl acetate/hexanes to 50% ethyl acetate/hexanes to give the title compound as an off-white solid (740 mg, 2.1 mmol, 56%) m/z (M+H): 346.

Step 3. (Preparation of N-cyclohexyl-2-hydroxy-4-(4,4,5,5-tetramethyl-1,3,2-dioxaborolan-2-yl)benzamide): The title compound was prepared according to the method described for Example 109 from N-cyclohexyl-2-hydroxy-4-iodobenzamide (740 mg, 2.1 mmol) to give an off-white solid (750 mg, 2.1 mmol, 100%) m/z (M+H): 346.

Step 4. (Preparation of N-cyclohexyl-2-hydroxy-4-[4-(4-oxo-4,5,6,7-tetrahydro-1H-pyrrolo[3,2-c]pyridin-2-yl)pyridin-2-yl]benzamide trifluoroacetate): The title compound was prepared according to Example 2 from N-cyclohexyl-2-hydroxy-4-(4,4,5,5-tetramethyl-1,3,2-dioxaborolan-2-yl)benzamide (735 mg, 2.1 mmol) and 2-(2-chloropyridin-4-yl)-1,5,6,7-tetrahydro-4H-pyrrolo[3,2-c]pyridin-4-one (350 mg, 1.4 mmol) to give a yellow solid (190 mg, 0.35 mmol, 25%). ¹H NMR (300 MHz, DMSO-d₆) δ 12.98 (s, 1H), 12.24 (s, 1H), 8.69 (d, J=7.6 Hz, 1H), 8.61 (d, J=5.8 Hz, 1H), 8.35 (s, 1H), 8.08 (d, J=8.3 Hz, 1H), 7.80 (d, J=4.5 Hz, 1H), 7.64 (m, 2H), 7.41 (s, 1H), 7.17 (s, 1H), 3.84 (bs, 1H), 3.43 (t, J=6.8 Hz, 2H), 2.90 (t, J=6.6 Hz, 2H), 1.91-1.57 (m, 5H), 1.45-1.08 (m, 5H). HRMS calculated for C₂₅H₂₆N₄O₃ (MH⁺) 431.2078, found 431.2063. Anal. calculated for C₂₅H₂₆N₄O₃.1.0 TFA.1.4 H₂O C, 56.91; H, 5.27; N, 9.83. Found: C, 56.94; H, 5.07; N, 9.67.

### EXAMPLE 152

This example illustrates the production of 2-[2-(1H-pyrrol-1-yl)pyridin-4-yl]-1,5,6,7-tetrahydro-4H-pyrrolo[3,2-c]pyridin-4-one trifluoroacetate.

Step 1. (Preparation of pyrrole sodium salt).

Sodium hydride (60% disp, 630 mg, 15.8 mmol) was suspended in 10.0 mL of tetrahydrofuran, cooled to 0° C. and treated with pyrrole (1.0 g 14.9 mmol) in 5.0 mL of tetrahydrofuran. The reaction was allowed to warm to room temperature and stirred 30 minuets, then condensed to a brown solid and used as-is.

Step 2. (Preparation of 2-[2-(1H-pyrrol-1-yl)pyridin-4-yl]-1,5,6,7-tetrahydro-4H-pyrrolo[3,2-c]pyridin-4-one trifluoroacetate).

A solution of 2-(2-chloropyridin-4-yl)-1,5,6,7-tetrahydro-4H-pyrrolo[3,2-c]pyridin-4-one (250 mg, 1.0 mmol) in 8.0 mL of dimethylsulfoxide was treated with the sodium salt of pyrrole (550 mg, 6.0 mmol) and heated to 100° C. for 16 hours, cooled to room temperature added 5.0 mL of methanol and 1.0 mL of trifluorocacetic acid, filtered through a syringe filter (0.45 mμ), purified by rpHPLC, and lyophilized to give the title compound as a tan solid (170 mg, 0.43 mmol, 43%). ¹H NMR (400 MHz, DMSO-d₆) δ 11.96 (s, 1H), 8.31 (d, J=5.4 Hz, 1H), 7.90 (s, 1H), 7.71 (s, 1H), 7.46 (d, J=4.6 Hz, 1H), 7.24 (s, 1H), 7.08 (bs, 1H), 6.38 (s, 2H), 3.42 (t, J=6.6 Hz, 2H), 2.86 (t, J=6.8 Hz, 2H). HRMS calculated for C₁₆H₁₄N₄O₂ (MH⁺) 279.1240 found 279.1230. Anal. calculated for C₁₆H₁₄N₄O₂.1.0 TFA.0.35 H₂O C, 52.14; H, 3.81; N, 13.51. Found: C, 52.21; H, 3.87; N, 13.46.

### EXAMPLE 153

This example illustrates the production of 2-[2-(3-phenyl-1H-pyrazol-1-yl)pyridin-4-yl]-1,5,6,7-tetrahydro-4H-pyrrolo[3,2-c]pyridin-4-one.

3-Phenyl pyrazole (467 mg, 3.24 mmol) was carefully added in portions to a stirred suspension of 60% NaH in mineral oil (194 mg, 4.85 mmol) in DMF (5.00 mL). When gas evolution ceased, 2-(2-chloropyridin-4-yl)-1,5,6,7-tetrahydro-4H-pyrrolo[3,2-c]pyridin-4-one (200 mg, 0.81 mmol) was added in portions with gas evolution. The resulting mixture was heated overnight at 140° C., then was diluted with an equal volume of H₂O, filtered, and purified by reverse phase chromatography to give 19.5 mg of 2-[2-(3-phenyl-1H-pyrazol-1-yl)pyridin-4-yl]-1,5,6,7-tetrahydro-4H-pyrrolo[3,2-c]pyridin-4-one as a pale yellow solid that was characterized by analytical reverse phase HPLC, H-NMR, F-NMR, and MS. Calculated Exact Mass 355.1433; Found Positive Electrospray LC-MS, m/e 356.1 (M+H⁺).

### EXAMPLE 154

This example illustrates the production of methyl 6-[4-(4-oxo-4,5,6,7-tetrahydro-1H-pyrrolo[3,2-c]pyridin-2-yl)pyridin-2-yl]-2-naphthoate trifluoroacetate.

Step 1. (Preparation of methyl 6-(4,4,5,5-tetramethyl-1,3,2-dioxaborolan-2-yl)-2-naphthoate).

The title compound was prepared according to the method described for Example 109 from methyl 6-bromo-2-naphthoate (500 mg, 1.9 mmol) to give a tan solid (405 mg, 1.3 mmol, 68%). m/z (M+H): 313.2.

Step 2. (Preparation of methyl 6-[4-(4-oxo-4,5,6,7-tetrahydro-1H-pyrrolo[3,2-c]pyridin-2-yl)pyridin-2-yl]-2-naphthoate trifluoroacetate).

The title compound was prepared according to the method described for Example 2 from methyl 6-(4,4,5,5-tetramethyl-1,3,2-dioxaborolan-2-yl)-2-naphthoate (375 mg, 1.2 mmol) and 2-(2-chloropyridin-4-yl)-1,5,6,7-tetrahydro-4H-pyrrolo[3,2-c]pyridin-4-one (250 mg, 1.0 mmol) to afford a yellow solid (125 mg, 0.24 mmol, 24%). ¹H NMR (400 MHz, DMSO-d₆) δ 12.26 (s, 1H), 8.78 (s, 1H), 8.74-8.68 (m, 2H), 8.50 (s, 1H), 8.34 (s, 1H), 8.18 (d, J=8.6 Hz, 1H), 8.06 (d, J=8.6 Hz, 1H), 7.82 (m, 1H), 7.45 (s, 1H), 7.17 (s, 1H), 3.94 (s, 3H), 3.44 (t, J=6.8 Hz, 2H), 2.92 (t, J=6.8 Hz, 2H). HRMS calculated for C₂₄H₁₉N₃O₃ (MH⁺) 398.1499 found 398-1456. Anal. calculated for C₂₄H₁₉N₃O₃.1.0 TFA.3.0 H₂O C, 55.22; H, 4.63; N, 7.43. Found: C, 55.21; H, 4.28; N, 7.31.

### EXAMPLE 155

This example illustrates the production of 2-{2-[4-(2-hydroxyethyl)phenyl]pyridin-4-yl}-1,5,6,7-tetrahydro-4H-pyrrolo[3,2-c]pyridin-4-one trifluoroacetate.

Step 1. (Preparation of 2-[4-(4,4,5,5-tetramethyl-1,3,2-dioxaborolan-2-yl)phenyl]ethanol).

A solution of 4-bromophenethyl alcohol (2.5 g, 12.4 mmol) in 40 mL of tetrahydrofuran was cooled to −78° C., treated with n-butyl lithium (1.6 M im hexanes, 29.7 mL, 18.5 mmol) and stirred for one hour. The reaction was then treated with triisopropyl borate (4.3 mL, 18.6 mmol) in 10 mL of tetrahydrofuran, warmed to room temperature and stirred for 30 minuets. Then treated with 50 mL of 2 M hydrochloric acid solution for one hour, extracted with methylene chloride, dried over magnesium sulfate, filtered and condensed to an oil. Purified by flash chromatography (gradient: 5% methanol / methylene chloride to 20% methanol / methylene chloride to afford the title compound as a clear colorless oil (980 mg, 5.9 mmol, 47%).

Step 2. (Preparation of 2-{2-[4-(2-hydroxyethyl)phenyl]pyridin-4-yl}-1,5,6,7-tetrahydro-4H-pyrrolo[3,2-c]pyridin-4-one trifluoroacetate).

The title compound was prepared according to the method described for Example 2 from 2-[4-(4,4,5,5-tetramethyl-1,3,2-dioxaborolan-2-yl)phenyl]ethanol (151 mg, 0.9 mmol) and 2-(2-chloropyridin-4-yl)-1,5,6,7-tetrahydro-4H-pyrrolo[3,2-c]pyridin-4-one (150 mg, 0.6 mmol) to give a yellow solid (125 mg, 0.3 mmol, 46%). ¹H NMR (300 MHz, DMSO-d₆) δ 12.37 (s, 1H), 8.61 (d, J=6.0 Hz, 1H), 8.36 (s, 1H), 7.99 (d, J=8.3 Hz, 2H), 7.86 (d, J=4.8 Hz, 1H), 7.50 (s, 1H), 7.46 (d, J=8.3 Hz, 2H), 7.21 (s, 1H), 3.66 (t, J=6.9 Hz, 2H), 3.43 (t, J=6.8 Hz, 2H), 2.91 (t, J=6.8 Hz, 2H), 2.82 (t, J=6.8 Hz, 2H). HRMS calculated for C₂₀H₁₉N₃O₂ (MH⁺) 334.1550, found 334.1538. Anal. calculated for C₂₀H₁₉N₃O₂.1.1 TFA.0.9 H₂O C, 56.13; H, 4.64; N, 8.84. Found: C, 56.22; H, 4.76; N, 8.46.

### EXAMPLE 156

This example illustrates the production of N-cyclohexyl-2-fluoro-4-[4-(4-oxo-4,5,6,7-tetrahydro-1H-pyrrolo[3,2-c]pyridin-2-yl)pyridin-2-yl]benzamide trifluoroacetate.

Step 1. (Preparation of 4-bromo-N-cyclohexyl-2-fluorobenzamide): A solution of 4-bromo-2-fluoro carboxylic acid (500 mg, 2.3 mmol), EDCI (480 mg, 2.5 mmol), and 1-hydroxybenzotriazole (340 mg, 2.5 mmol) in 15 mL of methylene chloride was treated with diisopropylethyl amine (0.6 mL, 3.4 mmol) and cyclohexyl amine (0.29 mmol, 2.5 mmol), stirred 16 hours, poured into water and extracted with methylene chloride, washed with brine, dried over magnesium sulfate, filtered and condensed to give the title compound as an off-white solid (400 mg, 1.3 mmol, 60%). m/z (M+H): 300.

Step2. (Preparation of N-cyclohexyl-2-fluoro-4-(4,4,5,5-tetramethyl-1,3,2-dioxaborolan-2-yl)benzamide),

The title compound was prepared according to the method described for Example 109 from 4-bromo-N-cyclohexyl-2-fluorobenzamide (400 mg, 1.3 mmol) to give an off-whit solid (360 mg, 1.0 mmol, 80%). m/z (M+H): 259.

Step3. (Preparation of N-cyclohexyl-2-fluoro-4-[4-(4-oxo-4,5,6,7-tetrahydro-1H-pyrrolo[3,2-c]pyridin-2-yl)pyridin-2-yl]benzamide trifluoroacetate).

The title compound was prepared according to the method described for Example 2 from N-cyclohexyl-2-fluoro-4-(4,4,5,5-tetramethyl-1,3,2-dioxaborolan-2-yl)benzamide (180 mg, 0.7 mmol) and 2-(2-chloropyridin-4-yl)-1,5,6,7-tetrahydro-4H-pyrrolo[3,2-c]pyridin-4-one (355 mg, 1.0 mmol) to give a yellow solid (240 mg, 0.4 mmol, 44%). ¹H NMR (300 MHz, DMSO-d₆) δ 12.16 (s, 1H), 8.62 (d, J=5.6 Hz, 1H), 8.37 (s, 1H), 8.27 (d, J=8.0 Hz, 1H), 8.05 (m, 2H), 7.73 (m, 2H), 7.38 (s, 1H), 7.14 (s, 1H), 3.75 (bs, 1H), 3.43 (t, J=6.5 Hz, 2H), 2.90 (t, J=6.5 Hz, 2H), 1.89-1.53 (m, 5H), 1.40-1.05 (m, 5H). HRMS calculated for C₂₅H₂₅N₄O₂ (MH⁺) 433.2034, found 433.2043. Anal. calculated for C₂₅H₂₅N₄O₂.1.0 TFA.1.55 H₂O C, 56.45; H, 5.10; N, 9.75. Found: C, 56.48; H, 4.84; N, 9.62.

### EXAMPLE 157

This example illustrates the production of N-cyclohexyl-3-fluoro-4-[4-(4-oxo-4,5,6,7-tetrahydro-1H-pyrrolo[3,2-c]pyridin-2-yl)pyridin-2-yl]benzamide trifluoroacetate.

Step 1. (Preparation of 4-bromo-N-cyclohexyl-3-fluorobenzamide).

A solution of 4-bromo-3-fluoro carboxylic acid (500 mg, 2.3 mmol), EDCI (480 mg, 2.5 mmol), and 1-hydroxybenzotriazole (340 mg, 2.5 mmol) in 15 mL of methylene chloride was treated with diisopropylethyl amine (0.6 mL, 3.4 mmol) and cyclohexyl amine (0.29 mmol, 2.5 mmol), stirred 16 hours, poured into water and extracted with methylene chloride, washed with brine, dried over magnesium sulfate, filtered and condensed to give the title compound as an off-white solid (680 mg, 2.2 mmol, 97%%). m/z (M+H): 300.

Step2. (Preparation of N-cyclohexyl-3-fluoro-4-[4-(4-oxo-4,5,6,7-tetrahydro-1H-pyrrolo[3,2-c]pyridin-2-yl)pyridin-2-yl]benzamide trifluoroacetate).

A suspension of 4-bromo-N-cyclohexyl-3-fluorobenzamide (370 mg, 1.2 mmol), bis(pinacolato)diboron (340 mg, 1.3 mmol), potassium acetate (362 mg, 3.7 mmol) and dichloro[1,1′-bis(diphenylphosphino)ferrocene]palladium(II) dichloromethane adduct (45 mg, 0.06 mmol) in 6.0 mL of dimethylformamide was heated to 80° C. for two hours. The reaction was cooled to room temperature and treated with 2-(2-chloropyridin-4-yl)-1,5,6,7-tetrahydro-4H-pyrrolo[3,2-c]pyridin-4-one (200 mg, 0.8 mmol), tetrakis(triphenylphosphine)palladium (0) (40 mg, 0.04 mmol) and 1.0 mL of 2.0 M cesium carbonate and heated to 80° C. for 16 hours. The reaction was cooled to room temperature, treated with 1.0 mL of trifluoroacetic acid, filtered through a syring filter (0.45 μm), purified by rpHPLC and lyopholized to give the title compound as a yellow solid (210 mg, 0.4 mmol, 50%). ¹H NMR (300 MHz, DMSO-d₆) δ 12.16 (s, 1H), 8.62 (d, J=5.6 Hz, 1H), 8.36 (d, J=7.8 Hz, 1H), 8.07 (s, 1H), 7.91 (t, J=8.0 Hz, 1H), 7.83-7.72 (m, 3H), 7.20 (s, 1H), 7.10 (s, 1H), 3.73 (bs, 1H), 3.36 (t, J=6.7 Hz, 2H), 2.82 (t, J=6.8 Hz, 2H), 1.86-1.63 (m, 4H), 1.56 (m, 1H), 1.35-1.18 (m, 4H), 1.15-1.01 (m, 1H). HRMS calculated for C₂₅H₂₅FN₄O₂ (MH⁺) 433.2034, found 433.2052. Anal. calculated for C₂₅H₂₅FN₄O₂.1.1 TFA.1.65 H₂O C, 55.59; H, 5.04; N, 9.53. Found: C, 55.59; H, 4.96; N, 9.68.

### EXAMPLE 158

This example illustrates the production of 2-(2-{4-[(cyclohexylamino)methyl]phenyl}pyridin-4-yl)-1,5,6,7-tetrahydro-4H-pyrrolo[3,2-c]pyridin-4-one trifluoroacetate.

Step 1. (Preparation of N-(4-bromobenzyl)cyclohexanamine).

A solution of 4-bromobenzylbromide (1.0 g, 4.0 mmol) and potassium carbonate (1.0 g, 7.2 mmol) in 10 mL of dimethylformamide was treated with cyclohexylamine (0.59 mL, 5.2 mmol) and heated to 85 degrees celcius for 56 hours. The reaction contents were cooled to room temperature, poured into water, extracted with ethyl acetate, dried over magnesium sulfate, filtered and condensed. Purification by flash chromatography (gradient: 100% methylene chloride to 25% methanol/methylene chloride) afforded the title compound as a clear colorless oil (920 mg, 3.4 mmol, 47%). m/z (M+H): 269/271.

Step 2. (preparation of 4-[(cyclohexylamino)methyl]phenylboronic acid).

The title compound was prepared according to the procedure described for Example 155, Step 1 from N-(4-bromobenzyl)cyclohexanamine (920 mg, 3.4 mmol) to give an off-white solid (500 mg 2.1 mmol, 63%) m/z (M+H): 234.

Step3. (Preparation of 2-(2-{4-[(cyclohexylamino)methyl]phenyl}pyridin-4-yl)-1,5,6,7-tetrahydro-4H-pyrrolo[3,2-c]pyridin-4-one trifluoroacetate).

The title compound was prepared according to the method described for Example 2 using of 4-[(cyclohexylamino)methyl]phenylboronic acid (280 mg, 1.2 mmol) and 2-(2-chloropyridin-4-yl)-1,5,6,7-tetrahydro-4H-pyrrolo[3,2-c]pyridin-4-one (200 mg, 0.8 mmol) to give a yellow solid (130 mg, 0.25 mmol, 30%). ¹H NMR (300 MHz, DMSO-d₆) δ 12.20 (s, 1H), 8.85 (bs, 1H), 8.61 (d, J=5.6 Hz, 1H), 8.33 (s, 1H), 8.22 (d, J=8.0 Hz, 2H), 7.76-7.62 (m, 3H), 7.34 (s, 1H), 7.13 (s, 1H), 4.26 (s, 2H), 3.42 (t, J=6.5 Hz, 2H), 3.04 (bs, 1H), 2.89 (t, J=6.6 Hz, 2H), 2.13 (m, 2H), 1.79 (m, 2H), 1.63 (m, 1H), 1.43-1.04 (m, 5H). HRMS calculated for C₂₅H₂₈N₄O (MH⁺) 401.2336, found 401.2340. Anal. calculated for C₂₅H₂₈N₄O.2.0 TFA.0.1 H₂O C, 55.25; H, 4.82; N, 8.88. Found: C, 55.28; H, 4.79; N, 8.80.

### EXAMPLE 159

This example illustrates the production of 2-{2-[3-(3-hydroxypropyl)phenyl]pyridin-4-yl}-1,5,6,7-tetrahydro-4H-pyrrolo[3,2-c]pyridin-4-one trifluoroacetate.

Step 1. (Preparation of 3-(3-bromophenyl)propan-1-ol).

A solution of 3-(3-bromophenyl)propionic acid (5.0 g, 21.8 mmol) in 65 mL of tetrahydrofuran was cooled to zero degrees celcius and treated with a solution of borohydride tetrahydrofuran complex 1.0 M in tetrahydrofuran (24.0 mL, 24.0 mmol). The reaction was allowed to warm to room temperature, heated to reflux for 16 hours, cooled to room temperature and quenched by addition of water followed by 100 mL of 1N hydrochloric acid. The aqueous was then extracted with ethyl acetate, dried over magnesium sulfate, filtered and condensed to give the title compound as an oil (4.7 g, 21.8 mmol, 100%). m/z (M+H): 215/217.

Step 2. (Preparation of 3-(3-hydroxypropyl)phenylboronic acid): The title compound was prepared according to the procedure described for Example 155, Step 1 from 3-(3-bromophenyl)propan-1-ol (2.5 g, 11.6 mmol) to give a foam (920 mg, 5.1 mmol, 44%) m/z (M+H): 181.

Step 3. (Preparation of 2-{2-[3-(3-hydroxypropyl)phenyl]pyridin-4-yl}-1,5,6,7-tetrahydro-4H-pyrrolo[3,2-c]pyridin-4-one trifluoroacetate).

The title compound was prepared according to the method described for Example 2 from 3-(3-hydroxypropyl)phenylboronic acid (164 mg, 0.9 mmol) and 2-(2-chloropyridin-4-yl)-1,5,6,7-tetrahydro-4H-pyrrolo[3,2-c]pyridin-4-one (150 mg, 0.6 mmol) to give a yellow solid (125 mg, 0.3 mmol, 50%). ¹H NMR (300 MHz, DMSO-d₆) δ 12.36 (s, 1H), 8.62 (d, J=6.0 Hz, 1H), 8.35 (s, 1H), 7.98-7.85 (m, 3H), 7.54-7.40 (m, 3H), 7.21 (s, 1H), 3.45 (m, 4H), 2.92 (t, J=6.6 Hz, 2H), 2.74 (t, J=7.6 Hz, 2H), 1.80 (m, 2H). HRMS calculated for C₂₁H₂₁N₃O₂ (MH⁺) 348.1707, found 348.1708. Anal. calculated for C₂₁H₂₁N₃O₂.1.2 TFA.1.7 H₂O C, 54.58; H, 5.01; N, 8.16. Found: C, 54.58; H, 5.02; N, 8.16.

### EXAMPLE 160

This example illustrates the production of 2-[2-(2,6-difluorophenyl)pyridin-4-yl]-1,5,6,7-tetrahydro-4H-pyrrolo[3,2-c]pyridin-4-one trifluoroacetate.

Step 1. Preparation of di-tert-butyl 2-(2-chloropyridin-4-yI)-4-oxo-6,7-dihydro-1H-pyrrolo[3,2-c]pyridine-1,5(4H)-dicarboxylate.

A 1.0 M solution of di-tert-butyl dicarbonate in THF (30 mL, 30 mmol) was added to a mixture of 2-(2-chloropyridin-4-yl)-1,5,6,7-tetrahydro-4H-pyrrolo[3,2-c]pyridin-4-one (Example 1) (3.0 g, 12.1 mmol) and N,N-dimethylaminopyridine (305 mg, 1.21 mmol) in dimethylformamide (30 mL). After 2.5 hours, the reaction was partitioned between ethyl acetate and saturated ammonium chloride. The organic layer was washed with saturated lithium chloride, water and brine, dried (sodium sulfate), and concentrated to give an off-white solid. The solid was recrystallized from ethyl acetate to give di-tert-butyl 2-(2-chloropyridin-4-yl)-4-oxo-6,7-dihydro-1H-pyrrolo[3,2-c]pyridine-1,5(4H)-dicarboxylate as white crystals (4.22 g, 9.42 mmol, 78% yield). ¹H NMR (300 MHz, CDCl₃) δ 8.36 (d, J=5.1, 1H), 7.22 (s, 1H), 7.14 (d, J=4.9, 1H), 6.73 (s, 1H), 4.09 (t, J=6.4, 2H), 3.22 (t, J=6.6, 2H), 1.54 (s, 1H), 1.36 (s, 1H). HRMS calculated for C₂₂H₂₇ClN₃O₅ (MH⁺) 448.1634, found 448.1632.

Step 2. Preparation of 2-[2-(2,6-difluorophenyl)pyridin-4-yl]-1,5,6,7-tetrahydro-4H-pyrrolo[3,2-c]pyridin-4-one trifluoroacetate.

A solution of 2,6-difluorobromobenzene (181 mg, 0.937 mmol) in tetrahydrofuran (4 mL) was cooled to −78° C. under nitrogen. n-Butyllithium (1.6 M in hexanes, 0.680 mL, 1.09 mmol) was added dropwise, and the resulting solution was stirred for 20 min. A solution of zinc chloride (0.5 M in tetrahydrofuran, 2.2 mL, 1.09 mmol) was added dropwise. The solution was allowed to warm to room temperature over 30 min. A solution of di-tert-butyl 2-(2-chloropyridin-4-yl)-4-oxo-6,7-dihydro-1H-pyrrolo[3,2-c]pyridine-1,5(4H)-dicarboxylate (350 mg, 0.781 mmol) and tetrakis(triphenylphospine)palladium(0) (45 mg, 0.0391 mmol) in tetrahydrofuran (4 mL) was added to the reaction solution at room temperature. The reaction was heated to reflux for 4 hours. The reaction was quenched with saturated ammonium chloride and partitioned between ethyl acetate and water. The organic layer was washed with brine, dried (sodium sulfate), concentrated, and purified by flash chromatography (30→60% ethyl acetate/hexanes) to give di-tert-butyl 2-[2-(2,6-difluorophenyl)pyridin-4-yl]-4-oxo-6,7-dihydro-1H-pyrrolo[3,2-c]pyridine-1,5(4H)-dicarboxylate as a white foam (118 mg, 0.225 mmol, 29% yield). Di-tert-butyl 2-[2-(2,6-difluorophenyl)pyridin-4-yl]-4-oxo-6,7-dihydro-1H-pyrrolo[3,2-c]pyridine-1,5(4H)-dicarboxylate (118 mg, 0.225 mmol) was dissolved in 50% trifluoroacetic acid/dichloromethane (4 mL) and was stirred overnight at room temperature. The reaction was concentrated under a stream of nitrogen and purified by reverse-phase HPLC (acetonitrile/water/0.05% trifluoroacetic acid) to give 2-[2-(2,6-difluorophenyl)pyridin-4-yl]-1,5,6,7-tetrahydro-4H-pyrrolo[3,2-c]pyridin-4-one trifluoroacetate as a lyophilized light yellow solid (72 mg, 0.164 mmol, 73% yield). ¹H NMR (400 MHz, DMSO-d₆) δ 12.19 (s, 1H), 8.68 (d, J=5.7, 1H), 8.01 (s, 1H), 7.85 (dd, J=5.7, 1.6, 1H), 7.63 (tt, J=8.5, 6.7, 1H), 7.31 (t, J=8.0, 2H), 7.28 (d, J=2.2, 1H), 7.16 (s, 1H), 3.41 (t, J=6.9, 2H), 2.86 (t, J=6.9, 2H). HRMS calculated for C₁₈H₁₄F₂N₃O (MH⁺) 326.1099, found 326.1102.

### EXAMPLE 161

This example illustrates the production of 2-[2-(pentafluorophenyl)pyridin-4-yl]-1,5,6,7-tetrahydro-4H-pyrrolo[3,2-c]pyridin-4-one trifluoroacetate. The title compound was prepared from bromopentafluorobenzene and di-tert-butyl 2-(2-chloropyridin-4-yl)-4-oxo-6,7-dihydro-1H-pyrrolo[3,2-c]pyridine-1,5(4H)-dicarboxylate in the same manner as for 2-[2-(2,6-difluorophenyl)pyridin-4-yl]-1,5,6,7-tetrahydro-4H-pyrrolo[3,2-c]pyridin-4-one trifluoroacetate. ¹H NMR (400 MHz, DMSO-d₆) δ 12.05 (s, 1H), 8.64 (d, J=5.3, 1H), 7.92 (s, 1H), 7.74 (dd, J=5.4, 1.7, 1H), 7.11 (d, J=2.4, 1H), 7.10 (s, 1H), 3.40 (t, J=6.4, 2H), 2.84 (t, J=6.9, 2H). HRMS calculated for C₁₈H₁₁F₅N₃O (MH⁺) 380.0817, found 380.0798.

### EXAMPLE 162

This example illustrates the production of N-ethyl-5-[4-(4-oxo-4,5,6,7-tetrahydro-1H-pyrrolo[3,2-c]pyridin-2-yl)pyridin-2-yl]-1H-indole-2-carboxamide.

Step 1. Preparation of 5-[4-(4-oxo-4,5,6,7-tetrahydro-1H-pyrrolo[3,2-c]pyridin-2-yl)pyridin-2-yl]-1H-indole-2-carboxylic acid. Ethyl 5-bromo-1H-indole-2-carboxylate was converted to ethyl 5-(4,4,5,5-tetramethyl-1,3,2-dioxaborolan-2-yl)-1H-indole-2-carboxylate by the procedure described for Example 109. A mixture of 2-(2-chloropyridin-4-yl)-1,5,6,7-tetrahydro-4H-pyrrolo[3,2-c]pyridin-4-one (Example 1) (1.00 g, 4.04 mmol), ethyl 5-(4,4,5,5-tetramethyl-1,3,2-dioxaborolan-2-yl)-1H-indole-2-carboxylate (1.91 g, 6.06 mmol), tetrakis(triphenylphospine)palladium(0) (234 mg, 0.202 mmol), 2.0 M aqueous cesium carbonate (6.1 mL, 12.1 mmol), and dimethylformamide (14 mL) was stirred at 80° C. under nitrogen for 40 hours. The reaction was cooled to room temperature and filtered through celite. The filtrate was diluted with water and the pH was adjusted to 7 with 3 N HCl. The mixture was further diluted with water and filtered. The precipitate was suspended in methanol (20 mL) and treated with 1 N LiOH (8 mL) and water (6 mL). The mixture was stirred at 50° C. overnight. The reaction was diluted with water and made basic with aqueous NaOH. The aqueous layer was washed with ethyl acetate and methylene chloride. The pH of the aqueous layer was adjusted to pH 5 with 3 N HCl. The resultant precipitate was filtered and washed with water, ethanol, and ether to give 5-[4-(4-oxo-4,5,6,7-tetrahydro-1H-pyrrolo[3,2-c]pyridin-2-yl)pyridin-2-yl]-1H-indole-2-carboxylic acid as a green solid (1.08 g, 2.89 mmol, 72% yield). LC-MS (ES+) MH⁺=373. ¹H NMR (400 MHz, DMSO-d₆) δ 13.2 (s, 1H), 12.66 (s, 1H), 12.20 (s, 1H), 8.58 (d, J=6.3, 1H), 8.51 (s, 1H), 8.47 (s, 1H), 7.99 (dd, J=8.7, 1.5, 1H), 7.95 (d, J=5.0, 1H), 7.63 (d, J=8.7, 1H), 7.59 (s, 1H), 7.25 (s, 2H), 3.44 (td, J=6.5, 1.8, 2H), 2.93 (d, J=6.7, 2H).

Step 2. Preparation of N-ethyl-5-[4-(4-oxo-4,5,6,7-tetrahydro-1H-pyrrolo[3,2-c]pyridin-2-yl)pyridin-2-yl]-1H-indole-2-carboxamide. 5-[4-(4-Oxo-4,5,6,7-tetrahydro-1H-pyrrolo[3,2-c]pyridin-2-yl)pyridin-2-yl]-1H-indole-2-carboxylic acid and ethylamine were converted to N-ethyl-5-[4-(4-oxo-4,5,6,7-tetrahydro-1H-pyrrolo[3,2-c]pyridin-2-yl)pyridin-2-yl]-1H-indole-2-carboxamide by the method described for N-butyl-4-[4-(4-oxo-4,5,6,7-tetrahydro-1H-pyrrolo[3,2-c]pyridin-2-yl)pyridin-2-yl]benzamide trifluoroacetate. ¹H NMR (400 MHz, DMSO-d₆) δ 12.0 (s, 1H), 11.69 (s, 1H), 8.55 (t, J=5.7, 1H), 8.52 (d, J=5.3, 1H), 8.43 (s, 1H), 8.22 (s, 1H), 8.05 (dd, J=8.7, 1.7, 1H), 7.52-7.48 (m, 2H), 7.19 (d, J=1.7, 1H), 7.14 (d, J=2.2, 1H), 7.05 (s, 1H), 3.43 (td, J=6.8, 2.4, 2H), 3.34 (q, J=7.1, 2H), 2.88 (t, J=6.8, 2H), 1.16 (t, J=7.2, 3H). HRMS calculated for C₂₃H₂₂N₅O₂ (MH⁺) 400.1768, found 400.1800.

The following examples were prepared by the same method:

| Example | | Calculated | Found |
|---|---|---|---|
| No. | Compound Name(s) | (m + H) | (m + H) |
| 163 | 2-{2-[2-(morpholin-4-ylcarbonyl)- | 442.1874 | 442.1852 |
| | 1H-indol-5-yl]pyridin-4-yl}- | | |
| | 1,5,6,7-tetrahydro-4H-pyrrolo[3, | | |
| | 2-c]pyridin-4-one | | |
| 164 | 2-{2-[2-(pyrrolidin-1-ylcarbonyl)- | 426.1925 | 426.1958 |
| | 1H-indol-5-yl]pyridin-4-yl}- | | |
| | 1,5,6,7-tetrahydro-4H-pyrrolo[3, | | |
| | 2-c]pyridin-4-one trifluoroacetate | | |
| 165 | 2-[2-(2-{[(2R)-2-(pyrrolidin-1- | 509.266 | 509.2675 |
| | ylmethyl)pyrrolidin-1-yl]carbonyl}- | | |
| | 1H-indol-5-yl)pyridin-4-yl]-1,5,6, | | |
| | 7-tetrahydro-4H-pyrrolo[3, | | |
| | 2-c]pyridin-4-one hydrochloride | | |

### EXAMPLE 166

This example illustrates the production of 2-[2-(6,7-dihydro-5H-benzo[7]annulen-8-yl)pyridin-4-yl]-1,5,6,7-tetrahydro-4H-pyrrolo[3,2-c]pyridin-4-one trifluoroacetate.

Step 1. (Preparation of 8-Bromo-6,7-dihydro-5H-benzocycloheptene).

This compound was synthesized following a method reported in the literature (Paquette, L. A., Dahnke, K., Doyon, J., He, W., Wyant K., Friedrich, D., J. Org. Chem. 1991, 56, 6199-6205). ¹H NMR (300 MHz, CDCl₃) δ 7.17-7.03 (m, 4H), 6.94 (s, 1H), 2.95-2.79 (m, 4H), 2.00-2.89 (m, 2H).

Step 2. (Preparation of 6,7-dihydro-8-(4,4,5,5-tetramethyl-1,3,2-dioxaborolan-2-yl)-5H-benzocycloheptene).

To a mixture of 8-Bromo-6,7-dihydro-5H-benzocycloheptene (1.0 g, 4.48 mmol) obtained in step 1, bis(pinacolato)diboron and KOAc (1.32 g, 13.4 mmol) in DMSO (24 mL), was added PdCl₂dppf-CH₂Cl₂ (0.29 g, 0.35 mmol). The mixture was heated at 80° C. overnight. The cooled reaction mixture was diluted with CH₂Cl₂ (100 mL) and H₂O (20 mL). The aqueous phase was extracted with additional amount of CH₂Cl₂ (2×50 mL). The combined organic phase was dried (Na₂SO₄), filtered and concentrated. Purification by flash chromatography (eluent 9:1 hexanes/EtOAc) gave the desired pinacolboronate (1.22 g, quantitative).

Step 3. (Preparation of 2-[2-(6,7-dihydro-5H-benzo[7]annulen-8-yl)pyridin-4-yl]-1,5,6,7-tetrahydro-4H-pyrrolo[3,2-c]pyridin-4-one trifluoroacetate).

This compound was synthesized in 27% yield by the cross coupling of vinyl boronate intermediate from step 2 and 2-(2-chloropyridin-4-yl)-1,5,6,7-tetrahydro-4H-pyrrolo[3,2-c]pyridin-4-one following the general procedure described for Example 2. Purification of the crude product by flash chromatography (eluent 90:9:1 CH₂Cl₂/MeOH/concd NH₄OH) gave the title compound as a free base, which was converted to the corresponding trifluoroacetatic acid salt to give a yellow solid: mp 164-169° C.; ¹H NMR (300 MHz, DMSO-d₆) δ 12.42 (s, 1H), 8.57 (d, J=6.2 Hz, 1H), 8.23 (s, 1H), 7.91 (d, J=6.2 Hz, 1H), 7.58 (s, 1H), 7.45-7.38 (m, 2H), 7.34-7.19 (m, 4H), 3.45 (td, J=6.6, 1.7 Hz, 2H), 2.92 (t, J=6.7 Hz, 2H), 2.88-2.75 (m, 4H), 2.21-2.08 (m, 2H); ESI-MS m/z 356 [M+H]⁺.

### EXAMPLE 167

This example illustrates the production of 2-[2-(1H-inden-2-yl)pyridin-4-yl]-1,5,6,7-tetrahydro-4H-pyrrolo[3,2-c]pyridin-4-one trifluoroacetate.

Step 1. (Preparation of 1H-inden-2-yl trifluoromethanesulfonate).

This compound was synthesized from 2-indanone following a procedure published in J. Med. Chem. 1996, 39, 3875-3877 using 2-indanone. The crude product was used in the next step without purification: ¹H NMR (300 MHz, CDCl₃) δ 8.05 (s, 1H), 7.43-7.20 (m, 4H), 6.68 (s, 1H), 3.66 (s, 2H).

Step 2. (Preparation of 2-(4,4,5,5-tetramethyl-1,3,2-dioxaborolan-2-yl)-indene).

This compound was synthesized following a procedure similar to the one described in step 2 of the synthesis of Example 166 using the 1H-inden-2-yl trifluoromethanesulfonate obtained in step 1 above. The crude product was used in the next step without purification.

Step 3. (Preparation of 2-[2-(1H-inden-2-yl)pyridin-4-yl]-1,5,6,7-tetrahydro-4H-pyrrolo[3,2-c]pyridin-4-one trifluoroacetate).

This compound was synthesized in 12% yield by the cross coupling of the vinyl boronate intermediate from step 2 and 2-(2-chloropyridin-4-yl)-1,5,6,7-tetrahydro-4H-pyrrolo[3,2-c]pyridin-4-one following the general procedure described for Example 2: mp 208-213° C.; ¹H NMR (300 MHz, DMSO-d₆) δ 12.40 (s, 1H), 8.58 (d, J=6.1 Hz, 1H), 8.34 (s, 1H), 8.04 (s, 1H), 7.83 (d, J=5.8 Hz, 1H), 7.67-7.53 (m, 3H), 7.42-7.31 (m, 2H), 7.26 (s, 1H), 4.05 (s, 2H), 3.48-3.42 (m, 2H), 2.94 (t, J=6.7 Hz, 2H); ESI-MS m/z 328 [M+H]⁺.

The following compounds were made in the same manner:

| Example | | Calculated | Found |
|---|---|---|---|
| No. | Compound Name(s) | (m + H) | (m + H) |
| 168 | 2-(2-cyclohex-1-en-1-ylpyridin- | 294.1606 | 294 |
| | 4-yl)-1,5,6,7-tetrahydro-4H- | | |
| | pyrrolo[3,2-c]pyridin-4-one | | |
| 169 | 2-(2-cyclohept-1-en-1-ylpyridin- | 308.1763 | 308 |
| | 4-yl)-1,5,6,7-tetrahydro-4H- | | |
| | pyrrolo[3,2-c]pyridin-4-one | | |

### EXAMPLE 170

This example illustrates the production of 2-[2-(6-Chloro-2H-chromen-3-yl)pyridin-4-yl]-1,5,6,7-tetrahydro-4H-pyrrolo[3,2-c]pyridin-4-one trifluoroacetate

Step 1. (Preparation of 3-bromo-6-chloro-2H-chromene).

To a solution of lithium acetate dihydrate (85 mg, 0.83 mmol) in 97:3 CH₃CN/H₂O (8.9 mL) was added 6-chloro-2H-1-benzopyran-3-carboxylic acid (0.88 g, 4.2 mmol), followed by NBS (0.78 g, 4.39 mmol) and the resultant suspension was stirred at room temperature overnight. The reaction mixture was concentrated to dryness under reduced pressure. Purification by flash column chromatography (eluent hexanes, then 95:5 hexanes/Et₂O) gave 3-bromo-6-chloro-2H-chromene (0.39 g, 38%) as a white solid: ¹H NMR (300 MHz, CDCl₃) δ 7.07 (dd, J=8.6, 2.5 Hz, 1H), 6.90 (d, J=2.5 Hz, 1H), 6.72 (d, J=8.8 Hz, 1H), 6.69 (s, 1H), 4.87 (d, J=1.6 Hz, 2H).

Step 2 (Preparation of 6-chloro-3-(4,4,5,5-tetramethyl-1,3,2-dioxaborolan-2-yl)-2H-chromene).

To a mixture of the pinacol diborane (0.43 g, 1.70 mmol), KOAc (0.45 g, 4.64 mmol) and 3-bromo-6-chloro-2H-chromene (0.38 g, 1.55 mmol) from step 1 was added DMSO (15.2 mL). The solution was degassed (3×, vacuum/argon), and PdCl₂dppf.CH₂Cl₂ (76 mg, 0.09 mmol) was added to it. The reaction mixture was degassed again (3×, vacuum/argon), and heated at 80° C. for 1 h. The cooled reaction mixture was diluted with CH₂Cl₂ (100 mL), washed with water (3×50 mL) and brine (50 mL), dried (Na₂SO₄), filtered and concentrated to give the crude vinyl boronate ester, which was used in the next step without further purification.

Step 3 (Preparation of 2-[2-(6-Chloro-2H-chromen-3-yl)pyridin-4-yl]-1,5,6,7-tetrahydro-4H-pyrrolo[3,2-c]pyridin-4-one trifluoroacetate).

This compound was synthesized in 11% yield by the cross coupling of the vinyl boronate intermediate from step 2 and 2-(2-chloropyridin-4-yl)-1,5,6,7-tetrahydro-4H-pyrrolo[3,2-c]pyridin-4-one following the general procedure described for Example 2: mp 197-201° C.; ¹H NMR (300 MHz, DMSO-d₆) δ 12.10 (br s, 1H), 8.52 (d, J=5.4 Hz, 1H), 8.09 (s, 1H), 7.64 (d, J=5.4 Hz, 1H), 7.56 (s, 1H), 7.32-7.23 (m, 3H), 7.12 (br s, 1H), 6.92 (d, J=8.6 Hz, 1H), 5.33 (s, 2H), 3.43 (t, J=6.8 Hz, 2H), 2.89 (t, J=6.6 Hz, 2H); ESI-MS m/z 378 [M+H]⁺.

### EXAMPLE 171

This example illustrates the production of 2-[2-(2H-chromen-3-yl)pyridin-4-yl]-1,5,6,7-tetrahydro-4H-pyrrolo[3,2-c]pyridin-4-one trifluoroacetate.

Step 1 (Preparation of 2H-chromene-3-carboxylic acid methyl ester).

To an ice-cold suspension of NaH (0.49 g, 12.4 mmol, 60% dispersion in oil) in THF (34.3 mL) was added salicylaldehyde (1.1 mL, 10.3 mmol) over 15 min. An additional volume of THF (10 mL) was added to the reaction mixture to facilitate stirring. After 2 h at 0° C., trimethyl-2-phosphonoacrylate (1.6 mL, 10.3 mmol) was added to it with vigorous shaking over 5 min. The ice-bath was removed, and the reaction mixture was stirred at room temperature for 2 h, and then at 70° C. for 2.5 h. The cooled reaction mixture was quenched with water, and the product was extracted into Et₂O (3×75 mL). The Et₂O extract was washed with water (100 mL) and brine, dried (Na₂SO₄), filtered and concentrated under reduced pressure. Purification by flash chromatography (eluent hexanes, then 99:1 to 93:7 hexanes/Et₂O) gave 2H-chromene-3-carboxylic acid methyl ester (1.07 g, 55%) as a white solid: ¹H NMR (300 MHz, CDCl₃) δ 7.44 (s, 1H), 7.23-7.20 (m, 1H), 7.13 (dd, J=7.2,1.6 Hz, 1H), 6.92 (td, J=7.4, 0.9 Hz, 1H), 6.84 (d, J=8.1 Hz, 1H), 5.00 (d, J=1.3 Hz, 2H), 3.82 (s, 3H).

Step2. (Preparation of 2H-chromene-3-carboxylic acid).

To an ice-cold solution of the ester (1.07 g, 5.63 mmol) from step 1 above in 2:1:1 THF/H₂O/MeOH (64 mL) was added lithium hydroxide dihydrate (0.47 g, 11.3 mmol). The ice-bath was removed, and the reaction mixture was heated under reflux for 35 min. The cooled reaction mixture was concentrated under reduced pressure, and acidified to pH 3-4 with concentrated HCl. The white precipitate formed was filtered, washed with water and Et₂O, and dried to give 2H-chromene-3-carboxylic acid (0.84 g, 85%), which was used in step 3 without further purification: ¹H NMR (300 MHz, DMSO-d₆) δ 7.45 (s, 1H), 7.34-7.31 (m, 1H), 7.25 (dd, J=7.9,1.5 Hz, 1H), 6.95 (td, J=7.4, 0.9 Hz, 1H), 6.85 (d, J=8.1 Hz, 1H), 4.91 (d, J=1.3 Hz, 2H).

Step 3. (Preparation of 3-bromo-2H-chromene).

This compound was prepared from 2H-chromene-3-carboxylic acid obtained in step 2 above by a procedure similar to the one described in step 1 of the synthesis of Example 170: ¹H NMR (300 MHz, CDCl₃) δ 7.14-7.10 (m, 1H), 6.94-6.88 (m, 2H), 6.79 (d, J=8.1 Hz, 1H), 6.75 (s, 1H), 4.88 (d, J=1.5 Hz, 2H).

Step 4. (Preparation of 3-(4,4,5,5-tetramethyl-1,3,2-dioxaborolan-2-yl)-2H-chromene).

This compound was prepared from 3-bromo-2H-chromene obtained in step 3 above by a procedure similar to the one described in step 2 of the synthesis of Example 170.

Step 5. (Preparation of 2-[2-(2H-chromen-3-yl)pyridin-4-yl]-1,5,6,7-tetrahydro-4H-pyrrolo[3,2-c]pyridin-4-one trifluoroacetate).

This compound was synthesized in 14% yield by the cross coupling of the vinyl boronate intermediate from step 4 and 2-(2-chloropyridin-4-yl)-1,5,6,7-tetrahydro-4H-pyrrolo[3,2-c]pyridin-4-one following the general procedure described for Example 2: mp 172-175° C.; ¹H NMR (300 MHz, DMSO-d₆) δ 12.10 (br s, 1H), 8.52 (d, J=5.5 Hz, 1H), 8.13 (s, 1H), 7.65 (d, J=5.7 Hz, 1H), 7.59 (s, 1H), 7.32-7.20 (m, 3H), 7.13 (br s, 1H), 7.01-6.96 (d, J=8.1 Hz, 1H), 6.90 (d, J=8.0 Hz, 1H), 5.30 (s, 2H), 3.43 (t, J=6.8 Hz, 2H), 2.89 (t, J=6.8 Hz, 2H); ESI-MS m/z 344 [M+H]⁺.

### EXAMPLE 172

This example illustrates the production of methyl 3-[4-(4-oxo-4,5,6,7-tetrahydro-1H-pyrrolo[3,2-c]pyridin-2-yl)pyridin-2-yl]quinoline-1(2H)-carboxylate trifluoroacetate.

Step 1 (Preparation of methyl 3-bromo-2H-quinoline-1-carboxylate).

To an ice-cold solution of 3-bromoquinoline (3.3 mL, 24.0 mmol) in Et₂O (24 mL) was added diisobutylaluminum hydride (25.9 mL, 25.9 mmol, 1.0 M solution in toluene) over 5 min. The reaction mixture was stirred at 0° C. for 3 h, and methyl chloroformate (6.0 mL, 78.1 mmol) was added in one portion to it. The ice-bath was removed, and the reaction mixture was stirred at room temperature overnight. The reaction mixture was poured into ice-water (250 mL) with vigorous stirring. Et₂O (200 mL) was added to the mixture, which was then stirred under N₂ for 1.5 h. The mixture was acidified to pH 1-2 with 6 N HCl, and the organic layer was separated out. The aqueous layer was re-extracted with CH₂Cl₂ (3×100 mL) and the combined organic extracts were washed with brine, dried (Na₂SO₄ and Na₂CO₃) and concentrated under reduced pressure. Purification by flash column chromatography (eluent 95:5 to 80:20 hexanes/EtOAc) gave methyl 3-bromo-2H-quinoline-1-carboxylate (3.97 g, 62%) as a yellow solid: ¹H NMR (300 MHz, CDCl₃) δ 7.60-7.50 (m, 1H), 7.24 (td, J=8.1, 1.7 Hz, 1H), 7.09 (td, J=7.4, 1.1 Hz, 1H), 7.02 (dd, J=7.6, 1.6 Hz, 1H), 6.81 (s, 1H), 4.61 (d, J=1.4 Hz, 2H), 3.81 (s, 3H).

Step 2. (Preparation of methyl 3-(4,4,5,5-tetramethyl-1,3,2-dioxaborolan-2-yl)-2H-quinoline-1-carboxylate).

This compound was prepared by a procedure similar to the one described in step 2 of the synthesis of Example 170 using methyl 3-bromo-2H-quinoline-1-carboxylate obtained in step 1 above. The isolated material was used without purification in the next step.

Step 3. (Preparation of methyl 3-[4-(4-oxo-4,5,6,7-tetrahydro-1H-pyrrolo[3,2-c]pyridin-2-yl)pyridin-2-yl]quinoline-1(2H)-carboxylate trifluoroacetate).

This compound was prepared in 5% yield by the cross coupling of the vinyl boronate intermediate from step 2 and 2-(2-chloropyridin-4-yl)-1,5,6,7-tetrahydro-4H-pyrrolo[3,2-c]pyridin-4-one following the general procedure described for Example 2.: mp 167-171° C.; ¹H NMR (300 MHz, DMSO-d₆) δ 12.10 (brs, 1H), 8.54 (d, J=5.5 Hz, 1H), 8.20 (s, 1H), 7.67-7.62 (m, 3H), 7.38-7.31 (m, 3H), 7.22-7.17 (m, 1H), 7.14 (brs, 1H), 4.92 (s, 2H), 3.74 (s, 3H), 3.44 (t, J=6.6 Hz, 2H), 2.90 (t, J=6.7 Hz, 2H); ESI-MS m/z 401 [M+H]⁺.

### EXAMPLE 173

This example illustrates the production of 2-[2-(1-glycoloyl-1,2-dihydroquinolin-3-yl)pyridin-4-yl]-1,5,6,7-tetrahydro-4H-pyrrolo[3,2-c]pyridin-4-one trifluoroacetate.

Step 1. (Preparation of 1-(2-acetyloxyacetyl)-3-bromo-1,2-dihydroquinoline).

To a solution of 3-bromoquinoline (1.0 mL, 7.2 mmol) in THF (144 mL) at −78° C. was added borane-THF complex (7.2 mL, 7.2 mmol, 1.0 M solution in THF). The reaction mixture was stirred at −78° C. for 30 min, and a solution of Red-Al (4.5 mL, 14.4 mmol, 65% in toluene) in THF (16 mL) was added to it. After another 30 in, acetoxyacetyl chloride (9.3 mL, 86.4 mmol) was added in one portion, and the cooling bath was removed. The reaction mixture was stirred at room temperature overnight, then cooled in an ice-bath, and quenched with water (15 mL). The precipitate formed was removed by filtration, and the product was partitioned between water (75 mL) and CH₂Cl₂ (150 mL). The organic layer was washed with brine, and concentrated under reduced pressure. Purification by flash column chromatography (eluent 90:10 to 70:30 hexanes/EtOAc) gave 1.56 g of 1-(2-acetyloxyacetyl)-3-bromo-1,2-dihydroquinoline as a clear oil: ¹H NMR (300 MHz, CDCl₃) δ 7.26-7.12 (m, 4H), 6.86 (s, 1H), 4.80 (s, 2H), 4.65 (s, 2H), 2.14 (s, 3H).

Step 2. (Preparation of 1-(2-acetyloxyacetyl)-3-(4,4,5,5-tetramethyl-1,3,2-dioxaborolan-2-yl)-1,2-dihydroquinoline).

This compound was prepared by a procedure similar to the one described in step 2 of the synthesis of 2-[2-(6-Chloro-2H-chromen-3-yl)pyridin-4-yl]-1,5,6,7-tetrahydro-4H-pyrrolo[3,2-c]pyridin-4-one trifluoroacetate using the 1-(2-acetyloxyacetyl)-3-bromo-1,2-dihydroquinoline obtained in step 1 above. The isolated material was used without purification in the next step.

Step 3. (Preparation of 2-[2-(1-glycoloyl-1,2-dihydroquinolin-3-yl)pyridin-4-yl]-1,5,6,7-tetrahydro-4H-pyrrolo[3,2-c]pyridin-4-one trifluoroacetate).

This compound was prepared in 5% yield by the cross coupling of the vinyl boronate intermediate from step 2 and 2-(2-chloropyridin-4-yl)-1,5,6,7-tetrahydro-4H-pyrrolo[3,2-c]pyridin-4-one following the general procedure described for Example 2: mp 144-148° C.; ¹H NMR (300 MHz, DMSO-d₆) δ 12.14 (brs, 1H), 8.56 (d, J=5.6 Hz, 1H), 8.19 (s, 1H), 7.66 (s, 2H), 7.62-7.60 (m, 1H), 7.44-7.28 (m, 4H), 7.14 (br s, 1H), 4.90 (s, 2H), 4.27 (s, 2H), 3.46-3.42 (m, 2H), 2.90 (t, J=6.7 Hz, 2H); ESI-MS m/z 401 [M+H]⁺.

### EXAMPLE 174

This example illustrates the production of 2-{2-[1-(2-hydroxy-2-methylpropanoyl)-1,2-dihydroquinolin-3-yl]pyridin-4-yl}-1,5,6,7-tetrahydro-4H-pyrrolo[3,2-c]pyridin-4-one trifluoroacetate.

Step 1. (Preparation of 1-(2-acetoxy-2-methylpropanoyl)-3-bromo-1,2-dihydroquinoline):

To a solution of 3-bromoquinoline (2.06 g, 9.9 mmol) in Et₂O (10 mL) at 0° C. was added a solution of diisobutylaluminum hydride (11 mL, 11 mmol, 1 M in hexanes). The resulting mixture was stirred at 0° C. for 4 h prior to the addition of (1-chlorocarbonyl-1-methyl)ethyl acetate (4.3 mL, 29.7 mmol). The reaction mixture was warmed to room temperature and stirred overnight. The mixture was diluted with water (20 mL) and acidified till pH 2 with 6 N HCl solution. The aqueous layer was extracted with CH₂Cl₂ (3×70 mL). The combined organic phase was dried (Na₂SO₄) and concentrated. The residue was purified by flash chromatography (eluent 1:1:1 CH₂Cl₂/hexanes/EtOAc) to give the title compound (4.25 g, 97%); ¹H NMR (300 MHz, CDCl₃) δ 7.41 (d, J=8.0 Hz, 1H), 7.33-7.24 (m, 1H), 7.18 (dt, J=7.4, 1.1 Hz, 1H), 7.11 (dd, J=7.5, 1.5 Hz, 1H), 6.89 (s, 1H), 4.57 (d, J=1.4 Hz, 2H), 2.05 (s, 3H), 1.51 (s, 6H).

Step 2. (Preparation of 1-(2-hydroxy-2-methylpropanoyl)-3-(4,4,5,5-tetramethyl-1,3,2-dioxaborolan-2-yl)-1,2-dihydroquinoline).

To a solution of the material (1.5 g, 4.4 mmol) obtained in step 1, bis-(pinacolato)diboron (1.12 g, 4.4 mmol), and KOAc (1.29 g, 13.14 mmol) in DMSO (23 mL) was added PdCl₂dppf (0.28 g, 0.35 mmol). The reaction mixture was heated to 90° C. for 2 h. The cooled reaction mixture was diluted with CH₂Cl₂ (75 mL) and H₂O (20 mL). The aqueous phase was extracted with additional CH₂Cl₂ (3×70 mL). The combined organic phase was dried (Na₂SO₄) and concentrated under reduced pressure. The residue was used without purification in the next step.

Step 3. (Preparation of 2-{2-[1-(2-hydroxy-2-methylpropanoyl)-1,2-dihydro quinolin-3-yl]pyridin-4-yl}-1,5,6,7-tetrahydro-4H-pyrrolo[3,2-c]pyridin-4-one trifluoroacetate).

This compound was prepared in 22% yield by the cross coupling of the vinyl boronate intermediate from step 2 and 2-(2-chloropyridin-4-yl)-1,5,6,7-tetrahydro-4H-pyrrolo[3,2-c]pyridin-4-one following the general procedure described for Example 2: mp 212-217° C.; ¹H NMR (300 MHz, DMSO-d₆) δ 12.16 (s, 1H), 8.58 (d, J=5.6 Hz, 1H), 8.20 (s, 1H), 7.68-7.74(m, 2H), 7.54 (d, J=7.9 Hz, 1H), 7.35-7.50 (m, 3H), 7.28 (dt, J=7.4, 0.9 Hz, 1H), 7.16 (brs, 1H), 5.07 (s, 1H), 4.91 (s, 2H), 3.44 (t, J=6.6 Hz, 2H), 2.91 (t, J=6.7 Hz, 2H), 1.35 (s, 6H).

### EXAMPLE 175

This example illustrates the production of N-(tert-butyl)-3-[4-(4-oxo-4,5,6,7-tetrahydro-1H-pyrrolo[3,2-c]pyridin-2-yl)pyridin-2-yl]quinoline-1 (2H)-carboxamide trifluoroacetate.

Step 1. (Preparation of N-(tert-butyl)-3-bromo-2H-quinoline-1-carboxamide).

This compound was prepared in 85% yield following a procedure similar the one described in step 1 of the synthesis of Example 172 using 3-bromoquinoline and tert-butyl isocyanate: ¹H NMR (300 MHz, CDCl₃) δ 12.16 (s, 1H),7.7.15-7.30 (m, 2H), 7.02-7.12 (m, 2H), 6.80 (d, J=1.0 Hz, 1H), 5.08 (s, 1H), 4.57 (d, J=1.3 Hz, 2H), 1.34 (s, 9H).

Step 2. (Preparation of N-(tert-butyl)-3-(4,4,5,5-tetramethyl-1,3,2-dioxaborolan-2-yl)-2H-quinoline-1-carboxamide).

This compound was prepared by a procedure similar to the one described in step 2 of the synthesis of Example 170 using the N-(tert-butyl)-3-bromo-2H-quinoline-1-carboxamide obtained in step 1 above. The isolated material was used without purification in the next step.

Step 3. (N-(tert-butyl)-3-[4-(4-oxo-4,5,6,7-tetrahydro-1H-pyrrolo[3,2-c]pyridin-2-yl)pyridin-2-yl]quinoline-1(2H)-carboxamide trifluoroacetate).

This compound was prepared in 17% yield by the cross coupling of the vinyl boronate intermediate from step 2 and 2-(2-chloropyridin-4-yl)-1,5,6,7-tetrahydro-4H-pyrrolo[3,2-c]pyridin-4-one following the general procedure described for Example 2: mp 198-203° C.; ¹H NMR (300 MHz, DMSO-d₆) δ 12.20 (s, 1H), 8.53 (d, J=5.6 Hz, 1H), 8.17 (s, 1H), 7.69 (d, J=5.2 Hz, 1H), 7.62 (s, 1H), 7.21-7.48 (m, 4H), 7.16 (s, 1H), 7.08 (t, J=7.4 Hz, 2H), 6.35 (s, 1H), 4.72 (s, 2H), 3.43 (t, J=6.5 Hz, 2H), 2.90 (d, J=6.7 Hz, 2H), 1.30 (s, 9H); ESI-MS m/z 442 [M+H]⁺.

### EXAMPLE 176

This example illustrates the production of 2-[2-(3-fluorophenyl)pyridin-4-yl]-3-nitro-1,5,6,7-tetrahydro-4H-pyrrolo[3,2-c]pyridin-4-one trifluoroacetate.

A solution of 2-[2-(3-fluorophenyl)pyridin-4-yl]-1,5,6,7-tetrahydro-4H-pyrrolo[3,2-c]pyridin-4-one trifluoroacetate (4.55 g, 15 mmol) in sulfuric acid (100 mL) was cooled to −7° C. and fuming nitric acid (0.75 mL) was added dropwise. After stirring for ten minutes the mixture was poured into ice water (1.5 L) and adjusted to pH 2 with 50% aq sodium hydroxide (approximately 350 mL). The mixture was filtered and the solid was stirred in hot water (40 mL) and acetonitrile (100 mL) and filtered to give 2-[2-(3-fluorophenyl)pyridin-4-yl]-3-nitro-1,5,6,7-tetrahydro-4H-pyrrolo[3,2-c]pyridin-4-one (2.0 g). The filtrate was purified by reverse phase chromatography to give 2-[2-(3-fluorophenyl)pyridin-4-yl]-3-nitro-1,5,6,7-tetrahydro-4H-pyrrolo[3,2-c]pyridin-4-one trifluoroacetate (35 mg). High resolution MS calculated for C₁₈H₁₄N₄O₃F₁ (M+H⁺)=353.1044. Found 353.1054

### EXAMPLE 177

This illustrates the production of 3-amino-2-[2-(3-fluorophenyl)pyridin-4-yl]-1,5,6,7-tetrahydro-4H-pyrrolo[3,2-c]pyridin-4-one trifluoroacetate. A solution of 2-[2-(3-fluorophenyl)pyridin-4-yl]-3-nitro-1,5,6,7-tetrahydro-4H-pyrrolo[3,2-c]pyridin-4-one (Example 176) (75% pure, 1.15 g) in concentrated hydrochloric acid (12 mL) was cooled to −5° C. and tin (II) chloride dihydrate (2.7 g) was added in portions over a 15 minute period. Water (15 mL) was added slowly with cooling and the mixture was filtered. The solid was washed with 6H HCl then acetonitrile. Purification by reverse phase chromatography gave 3-amino-2-[2-(3-fluorophenyl)pyridin-4-yl]-1,5,6,7-tetrahydro-4H-pyrrolo[3,2-c]pyridin-4-one trifluoroacetate as a yellow-orange solid (0.30 g). ). High resolution MS calculated for C₁₈H₁₆N₄O₁F₁ (M+H⁺)=323.1303. Found 323.1325.

### EXAMPLE 178

This example illustrates the production of 2-[2-(3-fluorophenyl)pyridin-4-yl]-4-oxo-4,5,6,7-tetrahydro-1H-pyrrolo[3,2-c]pyridine-3-diazonium trifluoroacetate. To a solution of 3-amino-2-[2-(3-fluorophenyl)pyridin-4-yl]-1,5,6,7-tetrahydro-4H-pyrrolo[3,2-c]pyridin-4-one trifluoroacetate (Example 177) (0.22 g) in water (2 mL) and trifluoroacetic acid (6 mL) was added a solution of sodium nitrite (55 mg) in water (0.5 mL). After five minutes the solution was diluted with water (20 mL), filtered, and purified by reverse phase chromatography to give 106 mg of 2-[2-(3-fluorophenyl)pyridin-4-yl]-4-oxo-4,5,6,7-tetrahydro-1H-pyrrolo[3,2-c]pyridine-3-diazonium trifluoroacetate as an orange solid (0.102 g). High resolution MS calculated for C₁₈H₁₃N₅O₁F₁ (M+)=334.1099, Found 334.1125 ¹H NMR (d₆-DMSO): δ 8.79 (d, J=5.2 Hz, 1H), 8.31 (s, 1H), 7.96 (d, J=8.0 Hz, 1H), 7.91 (dt, J=10.8 Hz, 2.4 Hz, 1H), 7.75 (dd, J=5.2, 0.8 Hz), 7.67 (s, 1H), 7.56 (td, J=8.0, 6.4 Hz, 1H), 3.46 (M, 2H), 2.87 (t, J=7.0 Hz, 2H) ¹⁹F NMR (d6-DMSO): −75.16 (s, TFA), −113.07 (m).

### EXAMPLE 179

This example illustrates the production of 3-fluoro-2-[2-(3-fluorophenyl)pyridin-4-yl]-1,5,6,7-tetrahydro-4H-pyrrolo[3,2-c]pyridin-4-one trifluoroacetate. 3-amino-2-[2-(3-fluorophenyl)pyridin-4-yl]-1,5,6,7-tetrahydro-4H-pyrrolo[3,2-c]pyridin-4-one (Example 177) (1.028 g, 4.0 mmol) was stirred in 50% aqueous tetrafluoroboric acid (21 mL) and a solution of sodium nitrite (0.301 g, 4.36 mmol) in water (1 mL) was added slowly with stirring. The mixture was poured into a petri dish and irradiated with a 450 watt UV lamp for four hours. The mixture was purified by reverse phase chromatography to give 3-fluoro-2-[2-(3-fluorophenyl)pyridin-4-yl]-1,5,6,7-tetrahydro-4H-pyrrolo[3,2-c]pyridin-4-one trifluoroacetate as a yellow solid (61 mg). High resolution MS calculated for C₁₈H₁₄N₃O₁F₂ (M+H⁺)=326.1099, Found 326.069. ¹H NMR (d6-DMSO): δ 12.06 (s, 1H), 8.63 (d, J=5.2 Hz, 1H), 8.13 (d, J=0.8 Hz, 1H), 7.91 (d, J=8.0 Hz, 1H), 7.87 (dt, J=10.4, 2.2 Hz, 1H), 7.56-7.62 (m, 2H), 7.33 (td, J=8.6, 2.3 Hz), 7.28 (bs, 1H), 3.39 (m, 2H), 2.85 (t, J=6.8 Hz, 2H).

### EXAMPLE 180

This example illustrates the production of 3-nitro-2-(2-quinolin-3-ylpyridin-4-yl)-1,5,6,7-tetrahydro-4H-pyrrolo[3,2-c]pyridin-4-one. To a solution of 2-(2-chloropyridin-4-yl)-1,5,6,7-tetrahydro-4H-pyrrolo[3,2-c]pyridin-4-one (2.47 g, 10 mmol) in concentrated sulfuric acid (50 mL) at −5° C. was added fuming nitric acid (dropwise). After 20 minutes the mixture was poured into ice water (500 mL). After stirring one half hour the mixture was filtered to give 2-(2-chloropyridin-4-yl)-3-nitro-1,5,6,7-tetrahydro-4H-pyrrolo[3,2-c]pyridin-4-one as a yellow solid. High resolution MS calculated for C₁₂H₁₀N₄O₃Cl₁ (M+H)=293.0436, Found 294.0410.

A mixture of 3-quinilineboronic acid (0.44 g, 2.56 mmol), 2-(2-chloropyridin-4-yl)-3-nitro-1,5,6,7-tetrahydro-4H-pyrrolo[3,2-c]pyridin-4-one (0.50 g, 1.7 mmol), 2M aqueous cesium carbonate (1.8 mL), dimethylformamide (6 mL) and tetrakis(triphenylphosphine)palladium (0) (0.14 g) was flushed with nitrogen and heated with stirring to 80° C. for 5 ½ hours. The mixture was filtered hot and was acidified (trifluoroacetic acid), dissolved in water/acetonitrile, and purified by reverse phase chromatography followed by crystallization from acetonitrile/water to give 3-nitro-2-(2-quinolin-3-ylpyridin-4-yl)-1,5,6,7-tetrahydro-4H-pyrrolo[3,2-c]pyridin-4-one (0.20 g) as a yellow solid. High resolution MS calculated for C₂₁H₁₆N₅O₃ (M+H⁺)=386.1248, Found 386.1267. ¹H NMR (d₆-DMSO): δ 12.47 (s, 1H), 9.62 (d, J=2.3 Hz, 1H), 9.03 (d, J=2.0 Hz, 1H), 8.80 (d, J=5.2 Hz, 1H), 8.31 (s, 1H), 8.10 (d, J=7.7 Hz, 1H), 8.07 (d, J=8.6 Hz, 1H), 7.81 (td, J 7.6, 1.1 Hz, 1H), 7.66 (td, J=7.4, 1.0 Hz, 1H), 7.52 (bs 1H), 7.46 (dd J=5.1, 1.6 Hz, 1H), 3.43 (td, J=6.8, 2.4 Hz, 2H), 2.86 (t, J=6.6 Hz, 2H).

### EXAMPLE 181

This example illustrates the production of 3-bromo-2-[2-(3-fluorophenyl)pyridin-4-yl]-1,5-dihydro-4H-pyrrolo[3,2-c]pyridin-4-one trifluoroacetate: A solution of 3-bromo-2-[2-(3-fluorophenyl)pyridin-4-yl]-1,5,6,7-tetrahydro-4H-pyrrolo[3,2-c]pyridin-4-one (2.0 g, 6.51 mmol) in concentrated sulfuric acid (10 mL) was cooled (ice bath) and fuming nitric acid (0.25 mL) was added dropwise. After two hours the mixture was poured into ice water (200 mL) and filtered to give a solid. Purification by reverse phase chromatography gave 3-bromo-2-[2-(3-fluorophenyl)pyridin-4-yl]-1,5-dihydro-4H-pyrrolo[3,2-c]pyridin-4-one trifluoroacetate as an orange solid. High resolution MS calculated for C₁₈H₁₂N₃O₁ Br₁F₁ (M+H⁺)=384.0142, 386.0124, Found 384.0187, 386.0150. ¹H NMR (d₆-DMSO): δ 12.43 (s, 1H), 10.95 (d, J=5.6 Hz, 1H), 8.75 (d, J=5.2 Hz, 1H), 8.34 (s, 1H), 7.88-7.97 (m, 2H), 7.56 (m, 1H), 7.30 (td, J=8.4, 2.4 Hz, 1H), 7.10 (t, J=6.4 Hz, 1H), 6.40 (d, J=6.8 Hz, 1H). ¹⁹F NMR (d₆-DMSO): −75.35 (s, TFA), −113.08 (m).

### EXAMPLE 182

This example illustrates the production of 3-bromo-2-[2-(2-fluorophenyl)pyridin-4-yl]-1,5,6,7-tetrahydro-4H-pyrrolo[3,2-c]pyridin-4-one trifluoroacetate.

A suspension of 2-[2-(2-fluorophenyl)pyridin-4-yl]-1,5,6,7-tetrahydro-4H-pyrrolo[3,2-c]pyridin-4-one trifluoroacetate (Example 65) (300 mg, 0.72 mmol) and triethylamine (0.10 mL, 0.72 mmol) in 5.0 mL of tetrahydrofuran was treated with N-bromosuccinimide (140 mg, 0.78 mmol). The reaction became homogeneous, stirred two hours, poured into water and extracted 3× with ethyl acetate, dried over magnesium sulfate and condensed. Dissolved residue in dimethylformamide, acidified with trifluoroacetic acid and purified by rpHPLC to give the title compound as a yellow solid (210 mg, 0.42 mmol, 58%). ¹H NMR (300 MHz, DMSO-d₆) δ 12.36 (s, 1H), 8.76 (d, J=5.5 Hz, 1H), 8.25 (s, 1H), 7.93 (t, J=7.9 Hz, 1H), 7.85 (d, J=5.3 Hz, 1H), 7.58-7.52 (m, 1H), 7.42-7.36 (m, 2H), 7.24 (s, 1H), 3.39 (t, J=6.4 Hz, 2H), 2.87 (t, J=6.6 Hz, 2H). HRMS calculated for C₁₈H₁₃BrFN₃O (MH⁺) 386.0299, 388.0280, found 386.0313, 388.0277. Anal. calculated for C₁₈H₁₃BrFN₃O.1.0 TFA.0.25 H₂O C, 47.59; H, 2.89; N, 8.32. Found: C, 47.63; H, 2.99; N, 8.43.

The following examples were prepared in the same manner:

| Example | | Calculated | Found |
|---|---|---|---|
| No. | Compound Name(s) | (m + H) | (m + H) |
| 183 | 3-bromo-2-[2-(3- | 386.0299 | 386.0294 |
| | fluorophenyl)pyridin-4-yl]-1,5, | | |
| | 6,7-tetrahydro-4H-pyrrolo[3, | | |
| | 2-c]pyridin-4-one trifluoroacetate | | |
| 184 | 3-bromo-2-[2-(2,4- | 404.0205 | 404.0197 |
| | difluorophenyl)pyridin-4-yl]-1, | | |
| | 5,6,7-tetrahydro-4H-pyrrolo[3, | | |
| | 2-c]pyridin-4-one trifluoroacetate | | |
| 185 | 2-[2-(2-fluorophenyl)pyridin-4- | 434.016 | 434.016 |
| | yl]-3-iodo-1,5,6,7-tetrahydro-4H- | | |
| | pyrrolo[3,2-c]pyridin-4-one | | |
| | trifluoroacetate | | |
| 186 | 3-chloro-2-[2-(2- | 342.0804 | 342.0835 |
| | fluorophenyl)pyridin-4-yl]-1,5, | | |
| | 6,7-tetrahydro-4H-pyrrolo[3, | | |
| | 2-c]pyridin-4-one trifluoroacetate | | |
| 187 | 3-chloro-2-[2-(2- | 340.0647 | 340.0667 |
| | fluorophenyl)pyridin-4-yl]-1, | | |
| | 5-dihydro-4H-pyrrolo[3, | | |
| | 2-c]pyridin-4-one trifluoroacetate | | |

### EXAMPLE 188

This example illustrates the production of 3-bromo-2-(2-quinolin-3-ylpyridin-4-yl)-1,5,6,7-tetrahydro-4H-pyrrolo[3,2-c]pyridin-4-one trifluroacetate.

A solution of 2-(2-quinolin-3-ylpyridin-4-yl)-1,5,6,7-tetrahydro-4H-pyrrolo[3,2-c]pyridin-4-one trifluoroacetate (500 mg, 1.47 mmol) in 10.0 mL of dimethylformamide was cooled to 0° C., treated with N-bromosuccinimide (260 mg, 1.47 mmol), and stirred for one hour. The reaction was allowed to warm to room temperature, then acidified by addition of trifluoroacetic acid and filtered through a syringe filter (0.45 μm). Purification by rpHPLC and lyopholization afforded the title compound as a yellow solid (340 mg, 0.64 mmol, 44%). ¹H NMR (400 MHz, DMSO-d₆) δ 12.34 (s, 1H), 9.61 (d, J=2.0 Hz, 1H), 9.08 (d, J=1.9 Hz, 1H), 8.74 (d, J=5.2 Hz, 1H), 8.43 (s, 1H), 8.14 (d,.J=7.5 Hz, 1H), 7.88 (dd, J=1.7 Hz, J=5.3 Hz, 1H), 7.83 (t, J=8.3 Hz, 1H), 7.68 (t, J=7.2 Hz, 1H), 7.20 (s, 1H), 3.36 (t, J=6.7 Hz, 2H), 2.85 (t, J=6.7 Hz, 2H). HRMS calculated for C₂₁H₁₅BrN₄O (MH⁺) 419.0502, 421.0484, found 419.0501, 421.0518. Anal. calculated for C₂₁H₁₅BrN₄O.1.3 TFA.1.0 H₂O C, 48.41; H, 3.15; N, 9.56. Found: C, 48.37; H, 3.16; N, 9.55.

The following examples were prepared in the same manner:

| Example | | Calculated | Found |
|---|---|---|---|
| No. | Compound Name(s) | (m + H) | (m + H) |
| 189 | 3-chloro-2-(2-quinolin-3-ylpyridin- | 375.1007 | 375.1045 |
| | 4-yl)-1,5,6,7-tetrahydro-4H- | | |
| | pyrrolo[3,2-c]pyridin-4- | | |
| | one trifluoroacetate | | |
| 190 | 3-iodo-2-(2-quinolin-3-ylpyridin- | 467.0363 | 467.0387 |
| | 4-yl)-1,5,6,7-tetrahydro-4H- | | |
| | pyrrolo[3,2-c]pyridin-4- | | |
| | one trifluoroacetate | | |

### EXAMPLE 191

This example illustrates the production of 3-methyl-2-(2-quinolin-3-ylpyridin-4-yl)-1,5,6,7-tetrahydro-4H-pyrrolo[3,2-c]pyridin-4-one trifluoroacetate.

A solution of 3-iodo-2-(2-quinolin-3-ylpyridin-4-yl)-1,5,6,7-tetrahydro-4H-pyrrolo[3,2-c]pyridin-4-one trifluoroacetate (Example 190) (300 mg, 0.64 mmol), trimethyl boroxine (0.35 mL, 2.56 mmol), and palladium dichloride diphenylphosphinoferrocene (40 mg, 0.046 mmol) in 0.96 mL of 2.0M potassium phosphate and 5.0 mL of dimethylformamide was heated to 100° C. for 18 hours. The reaction was cooled to room temperature, filtered through a syringe filter (0.20 μm), acidified with trifluoroacetic acid, purified by rpHPLC and lyophilized to afford the title compound as a yellow solid (80 mg, 0.15 mmol, 24%). ¹H NMR (300 MHz, DMSO-d₆) δ 11.90 (s, 1H), 9.63 (d, J=2.0 Hz, 1H), 9.11 (s, 1H), 8.74 (d, J=5.6 Hz, 1H), 8.29 (s, 1H), 8.18 (d, J=7.0 Hz, 1H), 7.88 (t, J=7.2 Hz, 1H), 7.74 (t, J=7.2 Hz, 1H), 7.65 (d, J=4.4 Hz, 1H), 7.09 (s, 1H), 3.40 (t, J=6.6 Hz, 2H), 2.87 (t, J=6.6 Hz, 2H). HRMS calculated for C₂₂H₁₈N₄O (MH⁺) 355.1553, found 355.1593. Anal. calculated for C₂₂H₁₈N₄O.1.5 TFA.0.35 H₂O C, 56.46; H, 3.82; N, 10.53. Found: C, 56.44; H, 3.99; N, 10.55.

### EXAMPLE 192

This example illustrates the production of 3-phenyl-2-(2-quinolin-3-ylpyridin-4-yl)-1,5,6,7-tetrahydro-4H-pyrrolo[3,2-c]pyridin-4-one trifluoroacetate.

A solution of 3-iodo-2-(2-quinolin-3-ylpyridin-4-yl)-1,5,6,7-tetrahydro-4H-pyrrolo[3,2-c]pyridin-4-one trifluoroacetate (Example 190) (300 mg, 0.64 mmol), phenyl boronic acid (118 mg, 0.96 mmol) and tetrakis(triphenylphosphine)palladium (0) (52 mg, 0.045 mmol) in 1.9 mL of 2.0M cesium carbonate and 5.0 mL of dimethyformamide was heated to 100° C. for 3 hours then at room temperature for 16 hours. The reaction was filtered through a syringe filter (0.20 μm) and acidified with trifluoroacetic acid, purified by rpHPLC and lyophilized to give the title compound as a yellow solid (110 mg, 0.19 mmol, 30%). ¹H NMR (300 MHz, DMSO-d₆) δ 12.16 (s, 1H), 9.33 (d, J=2.0 Hz, 1H), 8.70 (s, 1H), 8.54 (d, J=5.4 Hz, 1H), 8.09 (t, J=7.4 Hz, 2H), 7.86 (m, 2H), 7.73 (m, 1H), 7.41 (m, 3H), 7.33 (m, 2H), 7.14 (d, J=4.0 Hz, 1H), 7.06 (s, 1H), 3.44 (t, J=6.4 Hz, 2H), 2.93 (t, J=6.6 Hz, 2H). HRMS calculated for C₂₇H₂₀N₄O (MH⁺) 417.1710, found 417.1749. Anal. calculated for C₂₇H₂₀N₄O.1.25 TFA.0.25 H₂O C, 62.87; H, 3.89; N, 9.94. Found: C, 62.94; H, 3.90; N, 9.85.

The following example was prepared in the same manner:

| Example | | Calculated | Found |
|---|---|---|---|
| No. | Compound Name(s) | (m + H) | (m + H) |
| 193 | 2-(2-quinolin-3-ylpyridin-4-yl)- | 423.1274 | 423.1299 |
| | 3-thien-3-yl-1,5,6,7-tetrahydro- | | |
| | 4H-pyrrolo[3,2-c]pyridin-4- | | |
| | one trifluoroacetate | | |

### EXAMPLE 194

This example illustrates the production of 2-(2-quinolin-3-ylpyridin-4-yl)-5,6,7,8-tetrahydropyrrolo[3,2-c]azepin-4(1H)-one trifluoroacetate. Azepane-2,4-dione (Chem. Pharm. Bull. 19 (3) 529-534 (1971)) (2.0 g, 15.7 mmol), 2-bromo-1-(2-chloropyridin-4-yl)ethanone hydrobromide (4.96 g, 15.6 mmol) and ammonium acetate (4.8 g) were stirred in ethanol (75 mL) for 1.3 hours. Water (100 mL) was added and the mixture was concentrated to remove ethanol. The milky aqueous layer was decanted. After standing two hours the aqueous layer was filtered to give a yellow solid. The solid was washed with water, dried, washed with ether, and dried to give 2-(2-chloropyridin-4-yl)-5,6,7,8-tetrahydropyrrolo[3,2-c]azepin-4(1H)-one as a yellow solid (0.54 g). High resolution MS calculated for C₁₃H₁₃N₃O₁Cl₁ (M+H⁺)=262.0725, Found 262.0742 NMR (d₆-DMSO/D₂O): δ 8.24 (d, J=5.2 Hz, 1H), 7.56 (dd, J=5.2, 0.6 Hz, 1H), 7.12 (s, 1H), 3.15 (m, 2H), 1.93 (t, J=6.6 Hz, 2H), 1.89 (m, 2H).

A mixture of 3-quinilineboronic acid (0.39 g, 2.25 mmol), 2-(2-chloropyridin-4-yl)-5,6,7,8-tetrahydropyrrolo[3,2-c]azepin-4(1H)-one (0.39 g, 1.5 mmol), 2M aqueous cesium carbonate (2.25 mL), dimethylformamide (7.5 mL) and tetrakis(triphenylphosphine)palladium (0) (0.125 g) was flushed with nitrogen and heated with stirring to 80° C. for 8 hours. The mixture was filtered hot, diluted with water and acetonitrile, and purified by reverse phase chromatography to give 2-(2-quinolin-3-ylpyridin-4-yl)-5,6,7,8-tetrahydropyrrolo[3,2-c]azepin-4(1H)-one trifluoroacetate as a yellow solid (0.51 g). High resolution MS calculated for C₂₂H₁₉N₄O₁ (M+H⁺)=355.1553 Found 355.1569. ¹H NMR (d₆-DMSO): δ 11.88 (s, 1H), 9.66 (d, J=2.4, 1H), 9.16, (d, J=1.6, 1H), 8.67 (d, J=5.6 Hz, 1H), 8.51 (s, 1H), 8.14 (d, J=8.0 Hz, 1H), 8.11 (d, J=8.4 Hz, 1H), 7.86 (td, J=7.6, 1.4 Hz, 1H), 7.75 (dd, J=4.0 Hz, 1H), 7.71 (T, J=7.2 Hz, 1H), 7.49 (t, J=5.0 Hz, 1H), 7.44 (d, J=2.8 Hz, 1H), 3.19 (m, 2H), 3.01 (t, J=6.6 Hz, 2H), 1.95 (m, 2H).

### EXAMPLE 195

This example illustrates the production of 3-bromo-2-(2-quinolin-3-ylpyridin-4-yl)-5,6,7,8-tetrahydropyrrolo[3,2-c]azepin-4(1H)-one trifluoroacetate. 2-(2-quinolin-3-ylpyridin-4-yl)-5,6,7,8-tetrahydropyrrolo[3,2-c]azepin-4(1H)-one trifluoroacetate (Example 194) (0.27 g, 0.58 mmol), N-bromosuccinamide (0.126 g, 0.70 mmol), and triethylamine (0.09 mL, 0.65 mmol) were stirred in tetrahydrofuran (7 mL) for two hours. The mixture was diluted with water (30 mL) and filtered. The solid was purified by reverse phase chromatography to give 3-bromo-2-(2-quinolin-3-ylpyridin-4-yl)-5,6,7,8-tetrahydropyrrolo[3,2-c]azepin-4(1H)-one trifluoroacetate as a yellow solid (0.279 g). High resolution MS calculated for C₂₂H₁₈N₄O₁Br₁ (M+H⁺)=433.0658, Found 433.0680 ¹H NMR (d₆-DMSO): δ 12.11 (s, 1H), 9.66 (d, J=1.2 Hz, 1H), 9.12 (s, 1H), 8.77 (d, J=5,2 Hz, 1H), 8.46 (s, 1H), 8.18 (d, J=8.0 Hz, 1H), 8.11 (d, J=8.4 Hz, 1H), 7.92 (dd, J=5.6, 1.6 Hz, 1H), 7.86 (t, J=7.6 Hz, 1H), 7.71 (t, J=7.5 Hz, 1H), 7.64 (t, J=5.8 Hz, 1H), 3.10 (m, 2H), 2.96 (t, J=7.2 Hz, 2H), 1.94 (m, 2H).

### EXAMPLE 196

This example illustrates the production of 2-[2-(3-fluorophenyl)pyridin-4-yl]-5,6,7,8-tetrahydropyrrolo[3,2-c]azepin-4(1H)-one trifluoroacetate: A mixture of 3-fluorophenylboronic acid (0.387 g, 2.7 mmol), 2-(2-chloropyridin-4-yl)-5,6,7,8-tetrahydropyrrolo[3,2-c]azepin-4(1H)-one (0.471 g, 1.8 mmol), 2M aqueous cesium carbonate (2.25 mL), dimethylformamide (9 mL) and tetrakis(triphenylphosphine)palladium (0) (0.15 g) was flushed with nitrogen and heated with stirring to 80° C. for 8 hours. The mixture was diluted with water (5 mL), methanol (5 mL), and acetonitrile (5 mL) and was filtered hot purified by reverse phase chromatography to give a yellow solid (0.581 g). High resolution MS calculated for C₁₉H₁₇N₃O₁F₁ (M+H⁺)=322.1350 Found 322.1346.

### EXAMPLE 197

This example illustrates the production of 3-bromo-2-[2-(3-fluorophenyl)pyridin-4-yl]-5,6,7,8-tetrahydropyrrolo[3,2-c]azepin-4(1H)-one trifluoroacetate: 2-[2-(3-fluorophenyl)pyridin-4-yl]-5,6,7,8-tetrahydropyrrolo[3,2-c]azepin-4(1H)-one trifluoroacetate (Example 196) (0.255 g, 0.54 mmol), N-bromosuccinamide (0.125 g, 0.70 mmol), and triethylamine (0.12 mL, 0.86 mmol) were stirred in tetrahydrofuran (7 mL) for two hours. The mixture was diluted with water (60 mL) and acetonitrile (10 mL), acidified with trifluoroacetic acid, and filtered. The solution was purified by reverse phase chromatography to give a yellow solid (0.081 g). High resolution MS calculated for C₁₉H₁₆N₃O₁ Br₁F₁ (M+H⁺)=400.0493, 402.0437, Found 400.0493, 402.0416. ¹H NMR (d₆-DMSO): δ 12.05 (s, 1H), 8.68 (d, J=5.6 Hz, 1H), 8.24 (s, 1H), 7.93 (d, J=8.0 Hz, 1H), 7.86-7.91 (m, 2H), 7.54-7.63 (m, 2H), 7.31 (td, J=8.4, 1.2 Hz, 1H), 3.08 (m, 2H), 2.94 (t, J=7.4 Hz, 2H), 1.92 (m, 2H).

### EXAMPLE 198

This illustrates the procedure for the synthesis of 2-[2-(1H-indol-5-yl)pyridin-4-yl]-7-phenyl-1,5,6,7-tetrahydro-4H-pyrrolo[3,2-c]pyridin-4-one trifluoroacetate.

Step 1: Preparation of ethyl 3-amino-2-phenylpropanoate.

Ethyl cyano(phenyl)acetate (5 gm, 26.3 mmol) was dissolved in ethanol (100 ml), placed in Parr hydrogenator bottle and few drops of conc HCl were added. The solution was degassed, purged with nitrogen 3× times, 10% Pd/C on activated charcoal (1 gm) added. The solution stirred under hydrogen atmosphere (40 psi) overnight, filtered through celite, washed with ethanol and concentrated to give white solid. ¹H NMR (400 MHz, CD₃OD) δ 7.24 (m, 5H), 4.23 (q, 2H), 3.9 (m, 1H), 3.18 (m, 2H), 1.15 (t, 3H).m/z (M+H)=194.

Step 2: To a solution of 3-ethoxy-3-oxopropanoic acid (0.53 gm, 4 mmol) in dry dichloromethane (20 ml) was added EDC (0.92 gm, 4.8 mmol), HOBt (0.70 gm, 5.2 mmol), amine (0.772 gm, 4 mmol) from step 1, and NMO (2.7 gm, 26 mmol) at 0° C. The solution was stirred overnight, quenched with brine, diluted with dichloromethane, washed with 1.5N HCl, sat. NaHCO₃, brine and dried over Na₂SO₄ to give yellow oil (0.92 gm, 75%). ¹H NMR (400 MHz, CDCl3) δ 7.24 (m, 5H), 4.23 (m, 4H), 3.98 (m, 1H), 3.72 (m, 2H), 3.2 (s, 2H), 1.23 (t, 3H), 1.2(t, 3H). m/z (M+H)=308.

Step 3: 5-phenylpiperidine-2,4-dione.

To a solution of the amide (6 gm, 0.02 mol) from step 2 in toluene (100 ml) was added NaOMe (2 equi, 25% soln in MeOH) dropwise over 30 minutes. The solution heated at reflux overnight, quenched with water, org layer separated, washed with 1 M NaOH 2× times. The aqueous layers combined and made acidic with 1.5N HCl, extracted with EtOAc, dried over Na2SO4, filtered, concentrated to give solid used without further purification, m/z (M+H)=248. The solid dissolved in CH₂Cl₂, washed with 1.5N HCl, organic layer separated, dried over Na₂SO₄ and concentrated to give white solid (neutral compound). The solid dissolved in CH₃CN (50 ml) plus water (10 ml) and heated at reflux for 2 hours, solvent concentrated, ether added and conc. 3× times to give white solid used immediately. ¹H NMR (400 MHz, CDCl₃) δ 7.23 (m, 5H), 3.98 (m, 1H), 3.76 (m, 2H), 3.24 (s, 2H), m/z (M+H)=190.

Step 4: 2-(2-chloropyridin-4-yl)-7-phenyl-1,5,6,7-tetrahydro-4H-pyrrolo[3,2-c]pyridin-4-one.

2-bromo-1-(2-chloropyridin-4-yl)ethanone (1.2 mmol) was combined in absolute ethanol (30 ml) with ammonium acetate (6 mmol) and 5-phenylpiperidine-2,4-dione (1 mmol) from step 3. After 2 hours the mixture diluted with water (100 ml) to give brown solid, filtered, washed with water, followed by ethyl ether and dried under vacuum to give desired compound as brown solid. ¹H NMR (400 MHz, CD3OD) δ 8.27 (d, 1H), 7.73 (s, 1H), 7.63 (d, 1H), 7.23 (m, 5H), 7.08 (s, 1H), 4.2 (m, 1H), 3.92 (m, 1H), 3.45 (m, 1H). HRMS calculated for C₁₈H₁₅ClN₃O (M+H) 324.0898, found 324.0862.

Step 5: 2-[2-(1H-indol-5-yl)pyridin-4-yl]-7-phenyl-1,5,6,7-tetrahydro-4H-pyrrolo[3,2-c]pyridin-4-one trifluoroacetate: A suspension of 2-(2-chloropyridin-4-yl)-7-phenyl-1,5,6,7-tetrahydro-4H-pyrrolo[3,2-c]pyridin-4-one (150 mg, 0.46 mmol, 1 equiv) in dimethylformamide (10 mL) was treated with 1H-indol-5-ylboronic acid (1 mmol, 2 equiv) and 2.0 M cesium carbonate (652 mg, 2 mmol, in 1 mL water, 4 equiv). The reaction was purged with nitrogen (g) and degassed in vacuum 3× and then triphenylphosphinepalladium (57 mg, 0.05 mmol, 10 mol %) was added. The reaction was then heated to 100° C. overnight, solvent concentrated residue dissolved in water and acetonitrile, acidified with TFA and filtered through a syringe filter (0.45 μm), purified by prep. rpHPLC, and lyophilized to give the title compound as a yellow solid. ¹H NMR (400 MHz, CD₃OD) δ 8.95 (m, 1H), 8.34 (s, 1H), 7.93 (m, 1H) 7.70 (s, 1H), 7.42 (s, 1H), 7.40 (m, 1H), 7.25 (d, 1H), 7.23 (m, 5H), 6.64 (d, 1H), 4.2 (m, 1H), 3.92 (m, 1H), 3.45 (m, 1H), HRMS calculated for C₂₆H₂₀N₄O (M+H) 405.1710, found 405.1749.

The following examples were prepared in a similar manner as Example 198.

| Example | | Calculated | Found |
|---|---|---|---|
| No. | Compound Name(s) | (m + H) | m + H |
| 199 | 2-[2-(2-fluorophenyl)pyridin- | 384.1507 | 384.1495 |
| | 4-yl]-7-phenyl-1,5,6,7- | | |
| | tetrahydro-4H-pyrrolo[3, | | |
| | 2-c]pyridin-4-one trifluoroacetate | | |
| 200 | 2-[2-(1H-indol-5-yl)pyridin- | 405.171 | 405.1703 |
| | 4-yl]-6-phenyl-1,5,6,7- | | |
| | tetrahydro-4H-pyrrolo[3, | | |
| | 2-c]pyridin-4-one trifluoroacetate | | |
| 201 | 7-phenyl-2-(2-quinolin-3-ylpyridin- | 417.171 | 417.1711 |
| | 4-yl)-1,5,6,7-tetrahydro-4H- | | |
| | pyrrolo[3,2-c]pyridin-4- | | |
| | one trifluoroacetate | | |
| 202 | 4-[4-(7-methyl-4-oxo-4,5,6,7- | 348.1343 | 348.1364 |
| | tetrahydro-1H-pyrrolo[3, | | |
| | 2-c]pyridin-2-yl)pyridin- | | |
| | 2-yl]benzoic acid trifluoroacetate | | |
| 203 | methyl 4-[4-(7-methyl-4-oxo- | 362.1499 | 362.1511 |
| | 4,5,6,7-tetrahydro-1H-pyrrolo[3, | | |
| | 2-c]pyridin-2-yl)pyridin- | | |
| | 2-yl]benzoate trifluoroacetate | | |
| 204 | 2-[2-(4-aminophenyl)pyridin- | 319.1553 | 319.1545 |
| | 4-yl]-7-methyl-1,5,6,7-tetrahydro- | | |
| | 4H-pyrrolo[3,2-c]pyridin- | | |
| | 4-one trifluoroacetate | | |
| 205 | 2-(6′-fluoro-2,3′-bipyridin- | 323.1303 | 323.1288 |
| | 4-yl)-7-methyl-1,5,6,7-tetrahydro- | | |
| | 4H-pyrrolo[3,2-c]pyridin- | | |
| | 4-one trifluoroacetate | | |
| 206 | 7-methyl-2-{2-[4- | 350.1301 | 350.1322 |
| | (methylthio)phenyl]pyridin- | | |
| | 4-yl}-1,5,6,7-tetrahydro-4H- | | |
| | pyrrolo[3,2-c]pyridin-4- | | |
| | one trifluoroacetate | | |
| 207 | 7-methyl-2-{2-[4- | 382.122 | 382.123 |
| | (methylsulfonyl)phenyl]pyridin- | | |
| | 4-yl}-1,5,6,7-tetrahydro-4H- | | |
| | pyrrolo[3,2-c]pyridin-4- | | |
| | one trifluoroacetate | | |
| 208 | 2-fluoro-4-[4-(7-methyl-4-oxo-4, | 366.1248 | 366.1243 |
| | 5,6,7-tetrahydro-1H-pyrrolo[3, | | |
| | 2-c]pyridin-2-yl)pyridin- | | |
| | 2-yl]benzoic acid trifluoroacetate | | |
| 209 | 2-[2-(3-chlorophenyl)pyridin- | 338.1055 | 338.1081 |
| | 4-yl]-7-methyl-1,5,6,7-tetrahydro- | | |
| | 4H-pyrrolo[3,2-c]pyridin- | | |
| | 4-one trifluoroacetate | | |
| 210 | 2-[2-(3-chloro-4- | 356.096 | 356.0958 |
| | fluorophenyl)pyridin-4-yl]- | | |
| | 7-methyl-1,5,6,7-tetrahydro-4H- | | |
| | pyrrolo[3,2-c]pyridin-4- | | |
| | one trifluoroacetate | | |
| 211 | ethyl 2-fluoro-4-[4-(7-methyl- | 394.1561 | 394.1587 |
| | 4-oxo-4,5,6,7-tetrahydro-1H- | | |
| | pyrrolo[3,2-c]pyridin- | | |
| | 2-yl)pyridin-2-yl]benzoate | | |
| | trifluoroacetate | | |

### EXAMPLE 212

This example illustrates the preparation of 7-methyl-2-{2-[4-(piperidin-1-ylcarbonyl)phenyl]pyridin-4-yl}-1,5,6,7-tetrahydro-4H-pyrrolo[3,2-c]pyridin-4-one trifluoroacetate: To a solution of 4-[4-(7-methyl-4-oxo-4,5,6,7-tetrahydro-1H-pyrrolo[3,2-c]pyridin-2-yl)pyridin-2-yl]benzoic acid trifluoroacetate (0.1 mmol) in dry DMF (10 ml) was added CDI (0.3 mmol) and solution stirred at room temperature for 1 hour. Piperidine (0.5 mmol) was added dropwise and solution stirred overnight, solvent conc., residue purified by RPHPLC and lyophilized to give yellow solid. ¹H NMR (400 MHz, CD₃OD) δ 8.61 (d, 1H), 8.42 (s, 1H), 8.10 (d, 2H), 8.01 (d, 1H), 7.65 (d, 2H), 7.41 (s, 1H), 3.89 (m, 2H), 3.7 (m, 1H), 3.4 (m, 2H), 3.2 (m, 2H), 1.62 (m, 1H), 1.42 (d, 3H). HRMS calculated for C₂₅H₂₇N₄O₂ (M+H) 415.2129, found 415.2136.

The following examples were prepared by the method described for Example 212:

| Example | | Calculated | Found |
|---|---|---|---|
| No. | Compound Name(s) | (m + H) | m + H |
| 213 | 7-methyl-2-{2-[4-(pyrrolidin- | 401.1972 | 401.195 |
| | 1-ylcarbonyl)phenyl]pyridin- | | |
| | 4-yl}-1,5,6,7-tetrahydro-4H- | | |
| | pyrrolo[3,2-c]pyridin-4- | | |
| | one trifluoroacetate | | |
| 214 | 7-methyl-2-(2-{4- | 430.2238 | 430.2219 |
| | [(4-methylpiperazin- | | |
| | 1-yl)carbonyl]phenyl}pyridin- | | |
| | 4-yl)-1,5,6,7-tetrahydro-4H- | | |
| | pyrrolo[3,2-c]pyridin-4- | | |
| | one trifluoroacetate | | |
| 215 | 7-methyl-2-{2-[4-(morpholin- | 417.1921 | 417.191 |
| | 4-ylcarbonyl)phenyl]pyridin- | | |
| | 4-yl}-1,5,6,7-tetrahydro-4H- | | |
| | pyrrolo[3,2-c]pyridin- | | |
| | 4-one trifluoroacetate | | |
| 216 | N-cyclohexyl-N-methyl-4- | 443.2442 | 443.2411 |
| | [4-(7-methyl-4-oxo-4,5,6, | | |
| | 7-tetrahydro-1H-pyrrolo[3, | | |
| | 2-c]pyridin-2-yl)pyridin- | | |
| | 2-yl]benzamide trifluoroacetate | | |
| 217 | N-methyl-4-(4-oxo-4,5,6, | 271.119 | 271.1192 |
| | 7-tetrahydro-1H-pyrrolo[3, | | |
| | 2-c]pyridin-2-yl)pyridine- | | |
| | 2-carboxamide trifluoroacetate | | |
| 218 | N-methyl-4-[4-(7-methyl- | 361.1659 | 361.1656 |
| | 4-oxo-4,5,6,7-tetrahydro-1H- | | |
| | pyrrolo[3,2-c]pyridin- | | |
| | 2-yl)pyridin-2-yl]benzamide | | |
| | trifluoroacetate | | |
| 219 | N,N-dimethyl-4-[4-(7-methyl- | 375.1816 | 375.1812 |
| | 4-oxo-4,5,6,7-tetrahydro-1H- | | |
| | pyrrolo[3,2-c]pyridin- | | |
| | 2-yl)pyridin-2-yl]benzamide | | |
| | trifluoroacetate | | |
| 220 | 2-{2-[3-fluoro-4-(morpholin- | 435.1827 | 435.1808 |
| | 4-ylcarbonyl)phenyl]pyridin- | | |
| | 4-yl}-7-methyl-1,5,6, | | |
| | 7-tetrahydro-4H-pyrrolo[3, | | |
| | 2-c]pyridin-4-one trifluoroacetate | | |

### EXAMPLE 221

This example illustrates the preparation of 7-methyl-2-(6′-morpholin-4-yl-2,3′-bipyridin-4-yl)-1,5,6,7-tetrahydro-4H-pyrrolo[3,2-c]pyridin-4-one trifluoroacetate. To a solution of 2-(6′-fluoro-2,3′-bipyridin-4-yl)-7-methyl-1,5,6,7-tetrahydro-4H-pyrrolo[3,2-c]pyridin-4-one trifluoroacetate (35 mg, 1 mmol) in morpholine (5 ml) was added diisopropyl ethyl amine (1 ml) and solution heated at reflux overnight. The solvent concentrated, residue purified by RpHPLC and fractions lyophilized to give yellow solid. ¹H NMR (400 MHz, CD3OD) δ 8.71 (s, 1H), 8.51 (d, 1H), 8.3 (s, 1H), 8.11 (d, 1H), 7.89 (d, 2H), 7.41 (s, 1H), 7.01 (d, 1H), 3.65-3.95 (m, 9H), 3.2 (m, 2H), 1.56 (d, 3H). HRMS calculatedulated for C₂₂H₂₄N₅O₂ (M+H) 390.1925, found 390.1950.

The following examples were prepared by the method described for Example 221:

| Example | | Calculated | Found |
|---|---|---|---|
| No. | Compound Name(s) | (m + H) | m + H |
| 221 | 7-methyl-2-(6′-morpholin-4-yl- | 390.1925 | 390.195 |
| | 2,3′-bipyridin-4-yl)-1,5, | | |
| | 6,7-tetrahydro-4H-pyrrolo[3, | | |
| | 2-c]pyridin-4-one trifluoroacetate | | |
| 222 | 7-methyl-2-(6′-piperidin-1-yl- | 388.2132 | 388.2125 |
| | 2,3′-bipyridin-4-yl)-1,5, | | |
| | 6,7-tetrahydro-4H-pyrrolo[3, | | |
| | 2-c]pyridin-4-one trifluoroacetate | | |
| 223 | 7-methyl-2-[6′- | 403.2241 | 403.2259 |
| | (4-methylpiperazin-1-yl)-2, | | |
| | 3′-bipyridin-4-yl]-1,5, | | |
| | 6,7-tetrahydro-4H-pyrrolo[3, | | |
| | 2-c]pyridin-4-one trifluoroacetate | | |
| 224 | 2-(6′-thiomorpholin-4-yl-2, | 392.154 | 392.1533 |
| | 3′-bipyridin-4-yl)-1,5, | | |
| | 6,7-tetrahydro-4H-pyrrolo[3, | | |
| | 2-c]pyridin-4-one trifluoroacetate | | |
| 225 | 2-(6′-morpholin-4-yl-2, | 376.1768 | 376.1733 |
| | 3′-bipyridin-4-yl)-1,5, | | |
| | 6,7-tetrahydro-4H-pyrrolo[3, | | |
| | 2-c]pyridin-4-one trifluoroacetate | | |
| 226 | 2-[6′-(dimethylamino)-2, | 334.1662 | 334.1653 |
| | 3′-bipyridin-4-yl]-1,5, | | |
| | 6,7-tetrahydro-4H-pyrrolo[3, | | |
| | 2-c]pyridin-4-one trifluoroacetate | | |
| 227 | 2-(6′-piperidin-1-yl-2, | 374.1975 | 374.1968 |
| | 3′-bipyridin-4-yl)-1,5, | | |
| | 6,7-tetrahydro-4H-pyrrolo[3, | | |
| | 2-c]pyridin-4-one trifluoroacetate | | |
| 228 | 2-(6′-{[(1R)-1-phenylethyl]amino}- | 410.1975 | 410.1969 |
| | 2,3′-bipyridin-4-yl)-1,5, | | |
| | 6,7-tetrahydro-4H-pyrrolo[3, | | |
| | 2-c]pyridin-4-one trifluoroacetate | | |
| 229 | 2-(6′-{[(1S)-1-phenylethyl]amino}- | 410.1975 | 410.1972 |
| | 2,3′-bipyridin-4-yl)-1,5, | | |
| | 6,7-tetrahydro-4H-pyrrolo[3, | | |
| | 2-c]pyridin-4-one trifluoroacetate | | |
| 230 | tert-butyl 4-[4-(4-oxo-4,5, | 475.2452 | 475.2436 |
| | 6,7-tetrahydro-1H-pyrrolo[3, | | |
| | 2-c]pyridin-2-yl)-2,3′- | | |
| | bipyridin-6′-yl]piperazine-1- | | |
| | carboxylate trifluoroacetate | | |
| 231 | 2-(6′-piperazin-1-yl-2, | 375.1928 | 375.1951 |
| | 3′-bipyridin-4-yl)-1,5, | | |
| | 6,7-tetrahydro-4H-pyrrolo[3, | | |
| | 2-c]pyridin-4-one trifluoroacetate | | |
| 232 | 1,2-{6′-[(2-aminoethyl)amino]- | 349.1771 | 349.1783 |
| | 2,3′-bipyridin-4-yl}-1,5, | | |
| | 6,7-tetrahydro-4H-pyrrolo[3, | | |
| | 2-c]pyridin-4-one trifluoroacetate | | |
| 233 | 2-(2-morpholin-4-ylpyridin-4-yl)- | 299.1503 | 299.1501 |
| | 1,5,6,7-tetrahydro-4H-pyrrolo[3, | | |
| | 2-c]pyridin-4-one trifluoroacetate | | |
| 234 | 2-(2-morpholin-4-ylpyridin-4-yl)- | 449.2296 | 449.2286 |
| | 1,5,6,7-tetrahydro-4H-pyrrolo[3, | | |
| | 2-c]pyridin-4-one trifluoroacetate | | |
| 235 | 2-{2-[(2-aminoethyl) | 272.1506 | 272.1518 |
| | amino]pyridin-4-yl}-1,5, | | |
| | 6,7-tetrahydro-4H-pyrrolo[3, | | |
| | 2-c]pyridin-4-one trifluoroacetate | | |
| 236 | 4-(4-oxo-4,5,6,7-tetrahydro-1H- | 307.119 | 307.1185 |
| | pyrrolo[3,2-c]pyridin-2-yl)- | | |
| | 2,3′-bipyridin-6′(1′H)- | | |
| | one trifluoroacetate | | |
| 237 | 2-{6′-[(2-methoxyethyl) | 364.1768 | 364.1726 |
| | amino]-2,3′-bipyridin-4-yl}- | | |
| | 1,5,6,7-tetrahydro-4H-pyrrolo[3, | | |
| | 2-c]pyridin-4-one trifluoroacetate | | |
| 238 | 2-(2-thiomorpholin-4-ylpyridin- | 315.1274 | 315.1248 |
| | 4-yl)-1,5,6,7-tetrahydro-4H- | | |
| | pyrrolo[3,2-c]pyridin-4- | | |
| | one trifluoroacetate | | |

### EXAMPLE 239

This example illustrates the preparation of 6-[3-(benzyloxy)propyl]-2-[2-(2-fluorophenyl)pyridin-4-yl]-1,5,6,7-tetrahydro-4H-pyrrolo[3,2-c]pyridin-4-one trifluoroacetate.

Step 1. (Preparation of 4-(benzyloxy)butanal) 4-(benzyloxy)butanal was prepared by a literature method (Garcia, C. Martin, T., et. Al., J. Org. Chem., 66(4):1420 (2001)) from commercially available 4-(benzyloxy)butan-1-ol.

Step 2. (Preparation of ethyl (2E)-6-(benzyloxy)hex-2-enoate) To 4-(benzyloxy)butanal (6.4 g, 36.0 mmol) in dichloromethane was added (4-Ethoxycarbonyl)triphenylphosphonium chloride (18 g, 46.8 mmol), triethylamine (10.9 ml, 78.0 mmol) and the mixture was stirred overnight. The reaction mixture was treated with 100 ml water and the layers were separated. The organic layer was dried over sodium sulfate, filtered and concentrated in vacuo. The residue was purified by flash chromatography (0-15% ethyl acetate/hexanes) to give ethyl (2E)-6-(benzyloxy)hex-2-enoate (6.4 g, 72%) as a yellow oil. m/z (M+H): 249

Steps 3-6. (Preparation of 6-[3-(benzyloxy)propyl]-2-[2-(2-fluorophenyl)pyridin-4-yl]-1,5,6,7-tetrahydro-4H-pyrrolo[3,2-c]pyridin-4-one trifluoroacetate). The title compound was prepared from ethyl (2E)-6-(benzyloxy)hex-2-enoate in the same manner as for 6-[(Benzyloxy)methyl]-2-[2-(2-fluorophenyl)pyridin-4-yl]-1,5,6,7-tetrahydro-4H-pyrrolo[3,2-c]pyridin-4-one. ¹H NMR (400 MHz, DMSO-d₆) δ 1.23 (s, 1H), 8.67 (d, J=5.8 Hz, 1H), 8.13 (S, 1H), 7.87 (m, 2H), 7.59 (m, 1H), 7.43 (m, 2H), 7.31 (m, 6H), 7.18 (s, 1H), 4.45 (s, 2H), 3.68 (m, 1H), 3.44 (t, J=5.9 Hz, 2H), 2.99 (m, 1H), 2.69(m, 1H), 1.63 (m, 4H). HRMS calculated for C₂₈H₂₆FN₃O₂ (MH⁺) 456.2082, found 342.1344. Anal. calculated for C₂₈H₂₆FN₃O₂.1.0 TFA.0.50 H₂O C, 62.28; H, 4.88; N, 7.26. Found: C, 62.26; H, 4.80; N, 7.40.

### EXAMPLE 240

This example illustrates the preparation of 2-[2-(2-fluorophenyl)pyridin-4-yl]-6-(3-hydroxypropyl)-1,5,6,7-tetrahydro-4H-pyrrolo[3,2-c]pyridin-4-one trifluoroacetate.

The title compound was prepared from 6-[3-(benzyloxy)propyl]-2-[2-(2-fluorophenyl)pyridin-4-yl]-1,5,6,7-tetrahydro-4H-pyrrolo[3,2-c]pyridin-4-one in the same manner as for 2-[2-(2-fluorophenyl)pyridin-4-yl]-6-(hydroxymethyl)-1,5,6,7-tetrahydro-4H-pyrrolo[3,2-c]pyridin-4-one trifluoroacetate. ¹H NMR (400 MHz, DMSO-d₆) δ 12.24 (s, 1H), 8.67 (d, J=5.8 Hz, 1H), 8.14 (S, 1H), 7.87 (m, 2H), 7.61 (m, 1H), 7.47-7.39 (m, 2H), 7.31 (s, 1H), 7.17 (s, 1H), 3.67 (m, 1H), 3.41 (t, J=6.04 Hz, 2H), 2.96 (m, 1H), 2.68(m, 1H), 1.64-1.49 (m, 4H). HRMS calculated for C₂₁H₂₀FN₃O₂ (MH⁺) 366.1612, found 366.1624. Anal. calculated for C₂₁H₂₀FN₃O₂.1.1 TFA.0.10 H₂O C, 56.56; H, 4.36; N, 8.53. Found: C, 56.52; H, 4.30; N, 8.52.

### EXAMPLE 241

This example illustrates the preparation of 6-[(Benzyloxy)methyl]-2-[2-(2-fluorophenyl)pyridin-4-yl]-1,5,6,7-tetrahydro-4H-pyrrolo[3,2-c]pyridin-4-one.

Step 1. Preparation of ethyl 3-(allylamino)-4-(benzyloxy)butanoate. Ytterbium triflate (1.0 g, 2.35 mmol) was added to a solution of ethyl (2E)-4-(benzyloxy)but-2-enoate (Solladie et al. Tetrahedron Letters, 1987, 28, 61-64) (5.65 g, 25.7 mmol) and allylamine (5.8 mL, 77.1 mmol) in tetrahydrofuran (30 mL) at 0° C. . The reaction mixture was allowed to warm to room temperature overnight. After 16 hours, the mixture was diluted with ether and filtered through celite. The filtrate was concentrated and purified by flash chromatography (30→70% ethyl acetate/hexanes) to give ethyl 3-(allylamino)-4-(benzyloxy)butanoate as a golden oil (5.41 g, 19.5 mmol, 76% yield). LC-MS (ES+) MH⁺=278.

Step 2. Preparation of Ethyl 4-(benzyloxy)-3-[(3-ethoxy-3-oxopropanoyl)amino]butanoate. A mixture of ethyl 3-(allylamino)-4-(benzyloxy)butanoate (7.72 g, 27.8 mmol), N,N′-dimethylbarbituric acid (13.0 g, 83.5 mmol), tetrakis(triphenylphospine)palladium(0) (320 mg, 0.278 mmol) in dichloromethane (100 mL) was stirred at room temperature for 2 hours. The reaction mixture was concentrated to a slurry and partitioned between diethyl ether and saturated sodium bicarbonate. The ether layer was washed with sodium carbonate. The aqueous layers were re-extracted with diethyl ether. The ether layers were combined, dried (magnesium sulfate), and concentrated to give crude ethyl 3-amino-4-(benzyloxy)butanoate as a red oil (6.39 g). LC-MS (ES+) MH⁺=238.

1-(3-Dimethylaminopropyl)-3-ethylcarbodiimide hydrochloride (5.67 g, 29.6 mmol) was added to a solution of crude ethyl 3-amino-4-(benzyloxy)butanoate (6.39 g, 26.9 mmol), ethyl hydrogen malonate (3.91 g, 29.6 mmol), 1-hydroxybenzotriazole hydrate (4.00 g, 29.6 mmol), and triethylamine (4.13 mL, 29.6 mmol) in dichloromethane at 0° C. After 1 hour, the reaction was allowed to warm to room temperature overnight. The reaction mixture was diluted with ethyl acetate and washed with 1 N HCl, saturated sodium bicarbonate, and brine. The organic layers were dried (sodium sulfate), concentrated, and purified by flash chromatography (30→50% ethyl acetate/hexanes) to give ethyl 4-(benzyloxy)-3-[(3-ethoxy-3-oxopropanoyl)amino]butanoate as a golden oil (4.6 g, 13.1 mmol, 47% yield). LC-MS (ES+) MH⁺=352. ¹H NMR (400 MHz, CDCl₃) δ 7.57 (br d, 1H), 7.35-7.25 (m, 5H), 4.50 (s, 2H), 4.52-4.45 (m, 1H), 4.18 (q, J=7.1, 2H), 4.08 (q, J=7.1, 2H), 3.60-3.49 (AB of ABX, ν_{A}=3.58 ppm, ν_{B}=3.51 ppm, J_{AB}=9.4, J_{AX}=4.1, J_{BX}=5.5, 2H), 3.27 (s, 2H), 2.63 (t, J=6.3, 2H), 1.26 (t, J=7.2, 3H), 1.21 (t, J=7.1, 3H).

Step 3. Preparation of 6-[(Benzyloxy)methyl]piperidine-2,4-dione. A solution of sodium methoxide (25% in methanol, 9.0 mL, 39.3 mmol) was added to a solution of ethyl 4-(benzyloxy)-3-[(3-ethoxy-3-oxopropanoyl)amino]butanoate (4.60 g, 13.1 mmol) in methanol (10 mL). The reaction was refluxed for 3 hours, cooled to room temperature, and concentrated to give 6-[(benzyloxy)methyl]-3-carboxymethyl-4-hydroxy-2-oxo-1,2,5,6-tetrahydropyridine sodium salt as a foam. LC-MS (ES+), MH⁺=292. The foam was suspended in 4:1 acetonitrile/water (25 mL). Concentrated HCl (2.5 mL) was added, followed by citric acid (252 mg, 1.31 mmol). The pH was adjusted to 4 with 3 N HCl. The suspension was stirred at 80° C. for 5 hours. The reaction mixture was concentrated, and the remaining water was azeotroped with ethanol to give crude 6-[(benzyloxy)methyl]piperidine-2,4-dione as an oily solid. LC-MS (ES+) MH⁺=234.

Step 4. Preparation of 2-Bromo-1-[2-(2-fluorophenyl)pyridin-4-yl]ethanone hydrobromide. A mixture of 2-fluorophenylboronic acid (4.6 g, 32.6 mmol), 2-chloro-4-cyanopyridine (3.0 g, 21.7 mmol), tetrakis(triphenylphospine)palladium(0) (750 mg, 0.65 mmol) and 2.0 M aqueous sodium carbonate (32.6 mL, 65.1 mmol) in 2:1 ethylene glycol dimethyl ether/ethanol (90 mL) was refluxed for 90 min. The reaction was cooled, and air was bubbled through the reaction mixture. After the mixture turned brown, it was partitioned between ethyl acetate and water. The aqueous layer was extracted with ethyl acetate. The combined organic extracts were washed with brine, dried (sodium sulfate), concentrated, and purified by flash chromatography to give 2-(2-fluorophenyl)isonicotinonitrile as a white solid (3.86 g, 19.5 mmol, 90% yield). LC-MS (ES+) MH⁺=199. ¹H NMR (400 MHz, CDCl₃) δ 8.87 (dd, J=4.9, 0.8, 1H), 8.04 (d, J=1.0, 1H), 8.02 (td, J=7.8, 1.7, 1H), 7.46 (dd, J=5.0, 1.4, 1H), 7.46-7.40 (m, 1H), 7.28 (td, J=7.6, 1.0, 1H), 7.19 (ddd, J=11.5, 8.2, 1.0, 1H).

To a solution of 2-(2-fluorophenyl)isonicotinonitrile (500 mg, 2.52 mmol) in diethyl ether (5 mL) was added methylmagnesium bromide (3.0 M in diethyl ether, 0.925 mL, 2.77 mmol). The reaction was stirred at room temperature overnight. After 24 hours, the mixture was carefully poured into a mixture of ice (200 g) and 3 N HCl (100 mL). The solution was made slightly basic with 10% sodium hydroxide. The solution was extracted with diethyl ether. The ether layers were dried (magnesium sulfate), concentrated, and purified by flash chromatography (10→25% ethyl acetate/hexanes) to give 1-[2-(2-fluorophenyl)pyridin-4-yl]ethanone as a clear oil (370 mg, 1.72 mmol, 68% yield). LC-MS (ES+) MH⁺=216. ¹H NMR (400 MHz, CDCl₃) δ 8.89 (dd, J=5.1, 0.6, 1H), 8.22 (d, J=0.8, 1H), 7.99 (td, J=7.8, 1.8, 1H), 7.70 (dd, J=5.1, 1.7, 1H), 7.38-7.44 (m, 1H), 7.28 (td, J=7.5, 1.1, 1H), 7.18 (ddd, J=11.3, 8.3, 1.1, 1H), 2.66 (s, 3H).

To a solution of 1-[2-(2-fluorophenyl)pyridin-4-yl]ethanone (360 mg, 1.7 mmol) in glacial acetic acid (7 mL) was added bromine (0.090 mL, 1.75 mmol) and 30% HBr in acetic acid (0.333 mL, 1.67 mmol) at room temperature. After 90 minutes, the precipitate was filtered and washed with ether to give 2-bromo-1-[2-(2-fluorophenyl)pyridin-4-yl]ethanone hydrobromide as an off-white solid (502 mg, 1.7 mmol, quantitative). LC-MS (ES+) MH⁺=294, 296. ¹H NMR (400 MHz, DMSO-d₆) δ 8.96 (d, J=5.1, 1H), 8.20 (s, 1H), 7.94 (td, J=8.0, 1.8, 1H), 7.89 (dd, J=5.1, 1.6, 1H), 7.58-7.50 (m, 1H), 7.42-7.34 (m, 2H), 5.05 (s, 2H).

Step 5. Preparation of 6-[(Benzyloxy)methyl]-2-[2-(2-fluorophenyl)pyridin-4-yl]-1,5,6,7-tetrahydro-4H-pyrrolo[3,2-c]pyridin-4-one.

Crude 6-[(benzyloxy)methyl]piperidine-2,4-dione (Step 3) was treated with 2-bromo-1-[2-(2-fluorophenyl)pyridin-4-yl]ethanone hydrobromide (Step 4, 4.2 g, 14.4 mmol), ammonium acetate (4.0 g, 52.4 mmol), and ethanol (25 mL). The mixture was stirred at room temperature for 4 hours. The mixture was concentrated, and the resultant residue was diluted with water and ethyl acetate. Concentrated ammonium hydroxide was added until the solution was slightly basic. The product was extracted with ethyl acetate, and the organic layers were washed with brine, dried (sodium sulfate), concentrated, and purified by flash chromatography (50→95% ethyl acetate/hexanes/0.1% methanol) to provide 6-[(benzyloxy)methyl]-2-[2-(2-fluorophenyl)pyridin-4-yl]-1,5,6,7-tetrahydro-4H-pyrrolo[3,2-c]pyridin-4-one as a yellow solid (1.27 g, 2.97 mmol, 23% yield from ethyl 4-(benzyloxy)-3-[(3-ethoxy-3-oxopropanoyl)amino]butanoate, Step 2). ¹H NMR (400 MHz, DMSO-d₆) δ 11.97 (s, 1H), 8.58 (d, J=5.3, 1H), 7.94 (s, 1H), 7.86 (td, J=7.8, 1.8, 1H), 7.59 (dd, J=5.4, 1.7, 1H), 7.48-7.44 (m, 1H), 7.35-7.29 (m, 6H), 7.28-7.22 (m, 1H), 7.02-7.01 (m, 1H), 6.94 (d, J=2.0, 1H), 4.49 (s, 2H), 3.84 (m, X of ABX, 1H), 3.50-3.43 (AB of ABX, ν_{A}=3.48 ppm, ν=3.45 ppm, J_{AB}=7.8, J_{AX}=3.1, JBX=5.5, 2H), 3.02-2.82 (AB of ABX, ν_{A}=2.99 ppm, ν_{B}=2.86 ppm, J_{AB}=16.4, J_{AX}=6.0, J_{BX}=7.3, 2H). HRMS calculated for C₂₆H₂₃FN₃O₂ (MH⁺) 428.1769, found 428.1781.

### EXAMPLE 242

This example illustrates the preparation of 2-[2-(2-fluorophenyl)pyridin-4-yl]-6-(hydroxymethyl)-1,5,6,7-tetrahydro-4H-pyrrolo[3,2-c]pyridin-4-one trifluoroacetate.

Trimethylsilyl iodide (0.333 mL, 2.34 mmol) was added slowly to a solution of 6-[(benzyloxy)methyl]-2-[2-(2-fluorophenyl)pyridin-4-yl]-1,5,6,7-tetrahydro-4H-pyrrolo[3,2-c]pyridin-4-one (Example 241, 250 mg, 0.585 mmol) in chloroform (4.0 mL) at room temperature under nitrogen. After 40 hours, another portion of trimethylsilyl iodide (0.666 mL, 4.68 mmol) was added. Eight hours later, the reaction was quenched with methanol, treated with saturated sodium thiosulfite (2 mL), and concentrated. The residue was purified by reverse-phase HPLC (acetonitrile/water/0.05% trifluoroacetic acid) to furnish 2-[2-(2-fluorophenyl)pyridin-4-yl]-6-(hydroxymethyl)-1,5,6,7-tetrahydro-4H-pyrrolo[3,2-c]pyridin-4-one trifluoroacetate as a yellow solid (138 mg, 0.306 mmol, 52% yield). ¹H NMR (300 MHz, DMSO-d₆) δ 12.29 (s, 1H), 8.68 (d, J=5.9, 1H), 8.16 (s, 1H), 7.92-7.82 (m, 2H), 7.68-7.58 (m, 1H), 7.50-7.34 (m, 3H), 6.99 (s, 1H), 3.65 (m, 1H), 3.52-3.35 (m, 2H), 3.03-2.81 (m, 2H). HRMS calculated for C₁₉H₁₇FN₃O₂ (MH⁺) 338.1299, found 338.1321.

### EXAMPLE 244

This example illustrates the preparation of 7,7-Dimethyl-2-(2-quinolin-3-ylpyridin-4-yl)-1,5,6,7-tetrahydro-4H-pyrrolo[3,2-c]pyridin-4-one trifluoroacetate.

Step 1. Ethyl 3-[(tert-butoxycarbonyl)amino]-2-methylpropanoate

To the solution of ethyl 3-amino-2-methylpropanoate hydrochloride (14.7 g, 88 mmol) in acetonitrile (200 ml) was added di-tert-butyl dicarbonate (18.2 g, 83 mmol) and triethylamine (17.8 g, 175 mmol). The reaction mixture was stirred 18 hours at room temperature. Solid was filtered out. Filtrate was concentrated and brought up to ether (500 ml). The ether solution was washed with water (2×250 ml), brine (250 ml). Concentrated and dried under vacuum to give 16.8 g oil (83%). HRMS calculated for C₁₁H₂₁N₁O₄ (MH⁺) 232.1543, found 232.1579.

Step 2. Ethyl 3-[(tert-butoxycarbonyl)amino]-2,2-dimethylpropanoate

To the solution of ethyl 3-[(tert-butoxycarbonyl)amino]-2-methylpropanoate (6 g, 26 mmol) and iodomethane (11.1 g, 78 mmol) in anhydrous THF (200 ml) was added 52 ml of LDA (2 M in THF) at −78° C. The reaction mixture was stirred at −78° C. for 1.5 hours. The reaction was quenched with saturated NH4Cl and stirred at room temperature for 30 minutes. The solution was extracted with ether. Combined ether solution was concentrated. Concentrated residue was passed through short silica gel bed eluted with 100% hexane to 5% ethyl acetate in hexane to give 5.9 g oil (92%). HRMS calculated for C₁₂H₂₃N₁O₄ (MH⁺) 246.1700, found 246.1712.

Step 3. Ethyl 3-amino-2,2-dimethylproponate hydrochloride

Ethyl 3-[(tert-butoxycarbonyl)amino]-2,2-dimethylpropanoate (5.3 g, 21.6 mmol) was treated with 4N HCl in dioxane (20 ml). The reaction mixture was stirred 4 hours at room temperature. The reaction mixture was concentrated. Concentrated residue was suspended in ether and stirred for 30 minutes. Stripped off ether to give 3.6 g off white solid (92%). HRMS calculated for C₇H₁₅N₁O₂ (MH⁺) 146.1176, found 146.1164.

Step 4.

To a solution of ethyl 3-amino-2,2-dimethylproponate hydrochloride (3.6 g, 20 mmol) in dichloromethane (100 ml) was added triethylamine (2 g, 20 mmol) at 0° C. Ethyl hydrogen malonate (2.6 g, 20 mmol) in dichloromethane (25 ml) was added to the above solution followed by DCC (4.1 g, 20 mmol) in dichloromethane (25 ml) at 0° C. Ice bath was removed 30 minutes later and reaction was stirred at room temperature for 4 hours. Solid was filtered out and washed with dichloromethane (100 ml). Filtrate was washed with water, brine and dried over MgSO₄. Concentrated and dried to give 5.5 g of desired product. The material was used for the next reaction without further purification.

Step 5.

To the solution of the product from step 4 in toluene (150 ml) was added 5.7 ml Sodium mehoxide (25% in methanol). The reaction mixture was refluxed for 5 hours. The reaction was cooled to room temperature. Water (150 ml) was added to the reaction. Toluene layer was separated and extracted with water (2×100 ml). Combined aqueous solution was acidified with con. HCl. The acidified aqueous solution was extracted with dichloromethane (3×100 ml). Combined organic layer was concentrated and dried to give 2.8 g of the desired product. The crude material was used for the next reaction without further purification.

Step 6. 5,5-Dimethylpiperidine-2,4-dione.

The product (1.8 g) from step 5 was dissolved in acetonitrile (12 ml) and water (6 ml). The reaction mixture was refluxed for 4 hours. Cooled to room temperature. Concentrated and dried to give 1.4 g orange solid. HRMS calculated for C₇H₁₁N₁O₂ (MH⁺) 142.0863, found 142.0841.

Step 7. 2-(2-Chloropyridin-4-yl)-7,7-dimethyl-1,5,6,7-tetrahydro-4H-pyrrolo[3,2-c]pyridin-4-one.

HRMS calculated for C₁₄H₁₄Cl₁N₃O₁ (MH⁺) 142.0863, found 142.0841.

Step 8. 7,7-Dimethyl-2-(2-quinolin-3-ylpyridin-4-yl)-1,5,6,7-tetrahydro-4H-pyrrolo[3,2-c]pyridin-4-one trifluoroacetate. The title compound was prepared by the method described for Example 2.

HRMS calculated for C₂₃H₂₀N₄O₄ (MH⁺) 369.1710, found 369.1723.

The following examples were prepared by the same method as Example 243.

| Example | | Calculated | Found |
|---|---|---|---|
| No. | Compound Name(s) | (m + H) | (m + H) |
| 243 | 7,7-Dimethyl-2-(2-quinolin- | 369.1710 | 369.1723 |
| | 3-ylpyridin-4-yl)-1,5,6, | | |
| | 7-tetrahydro-4H-pyrrolo[3, | | |
| | 2-c]pyridin-4-one trifluoroacetate | | |
| 244 | 7-Ethyl-7-methyl-2-(2-quinolin- | 383.1866 | 383.1893 |
| | 3-ylpyridin-4-yl)-1,5,6, | | |
| | 7-tetrahydro-4H-pyrrolo[3, | | |
| | 2-c]pyridin-4-one trifluoroacetate | | |
| 245 | 2-[2-(2-Fluorophenyl)pyridin- | 336.1507 | 336.1512 |
| | 4-yl]-7,7-dimethyl-1,5,6, | | |
| | 7-tetrahydro-4H-pyrrolo[3, | | |
| | 2-c]pyridin-4-one trifluoroacetate | | |
| 246 | 7-Ethyl-2-[2-(2- | 350.1663 | 350.1640 |
| | fluorophenyl)pyridin-4-yl]- | | |
| | 7-methyl-1,5,6,7-tetrahydro- | | |
| | 4H-pyrrolo[3,2-c]pyridin- | | |
| | 4-one trifluoroacetate | | |
| 247 | 6-cyclopropyl-2-(2-quinolin- | 381.1710 | 381.1691 |
| | 3-ylpyridin-4-yl)-1,5,6, | | |
| | 7-tetrahydro-4H-pyrrolo[3, | | |
| | 2-c]pyridin-4-one trifluoroacetate | | |
| 248 | 6-cyclopropyl-2-[2-(2- | 348.1507 | 348.1504 |
| | fluorophenyl)pyridin-4-yl]- | | |
| | 1,5,6,7-tetrahydro-4H-pyrrolo[3,2- | | |
| 249 | N-cyclopentyl-3-[4- | 441.2285 | 441.2316 |
| | (6-cyclopropyl-4-oxo-4,5,6, | | |
| | 7-tetrahydro-1H-pyrrolo[3, | | |
| | 2-c]pyridin-2-yl)pyridin- | | |
| | 2-yl]benzamide trifluoroacetate | | |
| 250 | 2-[6′-(Dimethylamino)-2, | 348.1819 | 348.1833 |
| | 3′-bipyridin-4-yl]-6-methyl-1, | | |
| | 5,6,7-tetrahydro-4H-pyrrolo[3, | | |
| | 2-c]pyridin-4-one trifluoroacetate | | |
| 251 | 6-Methyl-2-(2-quinolin- | 355.1553 | 355.1524 |
| | 3-ylpyridin-4-yl)-1,5,6, | | |
| | 7-tetrahydro-4H-pyrrolo[3, | | |
| | 2-c]pyridin-4-one trifluoroacetate | | |
| 252 | 2-[2-(3,4,5-Trifluorophenyl)pyridin- | 322.1050 | 322.1378 |
| | 4-yl]-1,5,6,7-tetrahydro- | | |
| | 4H-pyrrolo[3,2-c]pyridin- | | |
| | 4-one trifluoroacetate | | |
| 253 | 2-(6′-Fluoro-2,3′- | 323.1303 | 323.1303 |
| | bipyridin-4-yl)-6-methyl-1,5, | | |
| | 6,7-tetrahydro-4H-pyrrolo[3, | | |
| | 2-c]pyridin-4-one trifluoroacetate | | |
| 254 | 3-Bromo-6-methyl-2-(2-quinolin- | 433.0658 | 433.0676 |
| | 3-ylpyridin-4-yl)-1,5,6, | | |
| | 7-tetrahydro-4H-pyrrolo[3, | | |
| | 2-c]pyridin-4-one trifluoroacetate | | |
| 255 | 2-[2-(2-Fluorophenyl)pyridin- | 322.1350 | 322.1365 |
| | 4-yl]-6-methyl-1,5,6, | | |
| | 7-tetrahydro-4H-pyrrolo[3, | | |
| | 2-c]pyridin-4-one trifluoroacetate | | |
| 256 | 6-Isopropyl-2-(2-quinolin- | 383.1866 | 383.1863 |
| | 3-ylpyridin-4-yl-1,5,6, | | |
| | 7-tetrahydro-4H-pyrrolol[3, | | |
| | 2-c]pyridin-4-one trifluoroacetate | | |
| 257 | 3-Bromo-2-[2-(2- | 400.0455 | 400.0464 |
| | fluorophenyl)pyridin- | | |
| | 4-yl]-6-methyl-1,5,6, | | |
| | 7-tetrahydro-4H-pyrrolo[3, | | |
| | 2-c]pyridin-4-one trifluoroacetate | | |
| 258 | 2-[2-(2-Fluorophenyl)pyridin- | 350.1663 | 350.1666 |
| | 4-yl]-6-isopropyl-1,5,6, | | |
| | 7-tetrahydro-4H-pyrrolo[3, | | |
| | 2-c]pyridin-4-one trifluoroacetate | | |
| 259 | 6-Phenyl-2-(2-quinolin- | 417.1710 | 417.1693 |
| | 3-ylpyridin-4-yl)-1,5,6, | | |
| | 7-tetrahydro-4H-pyrrolo[3, | | |
| | 2-c]pyridin-4-one trifluoroacetate | | |
| 260 | 2-(2-Chloropyridin-4-yl)-6- | 262.0742 | 262.0757 |
| | methyl-1,5,6,7-tetrahydro-4H- | | |
| | pyrrolo[3,2-c]pyridin-4-one | | |
| 261 | 2-[2-(3-Fluorophenyl)pyridin- | 384.1507 | 384.1498 |
| | 4-yl]-6-phenyl-1,5,6, | | |
| | 7-tetrahydro-4H-pyrrolo[3, | | |
| | 2-c]pyridin-4-one trifluoroacetate | | |
| 262 | 2-[2-(2-fluorophenyl)pyridin- | 384.1507 | 384.1518 |
| | 4-yl]-6-phenyl-1,5,6, | | |
| | 7-tetrahydro-4H-pyrrolo[3, | | |
| | 2-c]pyridin-4-one trifluoroacetate | | |
| 263 | 2-[2-(2-fluorophenyl)pyridin- | 376.1068 | 376.1071 |
| | 4-yl]-6-(trifluoromethyl)-1,5, | | |
| | 6,7-tetrahydro-4H-pyrrolo[3, | | |
| | 2-c]pyridin-4-one trifluoroacetate | | |
| 264 | 2-{2-[3-(methylthio) | 404.1039 | 404.1027 |
| | phenyl]pyridin-4-yl}- | | |
| | 6-(trifluoromethyl)-1,5,6, | | |
| | 7-tetrahydro-4H-pyrrolo[3, | | |
| | 2-c]pyridin-4-one trifluoroacetate | | |
| 265 | N-cyclopentyl-3-{4- | 469.1846 | 469.1825 |
| | [4-oxo-6-(trifluoromethyl)-4,5, | | |
| | 6,7-tetrahydro-1H-pyrrolo[3, | | |
| | 2-c]pyridin-2-yl]pyridin- | | |
| | 2-yl}benzamide trifluoroacetate | | |
| 266 | 2-[6′-(Dimethylamino)-2, | 402.1536 | 402.1532 |
| | 3′-bipyridin-4-yl]-6- | | |
| | (trifluoromethyl)-1,5,6, | | |
| | 7-tetrahydro-4H-pyrrolo[3, | | |
| | 2-c]pyridin-4-one trifluoroacetate | | |
| 267 | N-cyclopropyl-3-{4-[4- | 441.1533 | 441.1566 |
| | oxo-6-(trifluoromethyl)-4,5, | | |
| | 6,7-tetrahydro-1H-pyrrolo[3, | | |
| | 2-c]pyridin-2-yl]pyridin- | | |
| | 2-yl}benzamide trifluoroacetate | | |
| 268 | 7-Methyl-2-(2-quinolin-3-ylpyridin- | 355.1553 | 355.1584 |
| | 4-yl)-1,5,6,7-tetrahydro-4H- | | |
| | pyrrolo[3,2-c]pyridin- | | |
| | 4-one trifluoroacetate | | |
| 269 | 2-[2-(3-Fluorophenyl)pyridin- | 322.1350 | 322.1364 |
| | 4-yl]-7-methyl-1,5,6, | | |
| | 7-tetrahydro-4H-pyrrolo[3, | | |
| | 2-c]pyridin-4-one trifluoroacetate | | |
| 270 | 2-[2-(2-Fluorophenyl)pyridin- | 322.1350 | 322.1347 |
| | 4-yl]-7-methyl-1,5,6, | | |
| | 7-tetrahydro-4H-pyrrolo[3, | | |
| | 2-c]pyridin-4-one trifluoroacetate | | |

### EXAMPLE 271

This example illustrates preparation of irreversible inhibitors N-{4-[4-(4-oxo-4,5,6,7-tetrahydro-1H-pyrrolo[3,2-c]pyridin-2-yl)pyridin-2-yl]phenyl}acrylamide trifluoroacetate. To a stirred solution of 2-[2-(4-aminophenyl)pyridin-4-yl]-1,5,6,7-tetrahydro-4H-pyrrolo[3,2-c]pyridin-4-one trifluoroacetate (60 mg, 2 mmol) in dry THF (10 ml) at 0° C. was added diisopropyl ethyl amine (77 mg, 6 mmol) followed by DMF (1 ml) and acryloyl chloride (26 mg, 3 mmol). The solution was stirred for 30 minutes at 0° C., quenched with water, solvent evaporated, and residue purified by RpHPLC, fractions lyophilized to give yellow solid. ¹H NMR (400 MHz, CD3OD) δ 8.57 (d, 2H), 8.31 (d, 2H), 8.03 (s, 1H), 7.88 (d, 2H), 7.41 (s, 1H), 6.5 (m, 2H), 5.8 (m, 1H), 3.64 (t, 2H), 3.12 (t, 2H), HRMS calculated for C₂₁H₁₈N₄O₂ (M+H) 359.1503, found 359.1529.

The following examples were prepared in the same manner as Example 271.

| Example | | Calculated | Found |
|---|---|---|---|
| No. | Compound Name(s) | (m + H) | (m + H) |
| 272 | (2E)-N-{4-[4-(oxo-4,5, | 373.1659 | 373.1664 |
| | 6,7-tetrahydro-1H-pyrrolo[3, | | |
| | 2-c]pyridin-2-yl)pyridin- | | |
| | 2-yl]phenyl}but-2-enamide | | |
| | trifluoroacetate | | |
| 273 | 3-methyl-N-{4-[4-(4-oxo-4,5, | 387.1805 | 387.1816 |
| | 6,7-tetrahydro-1H-pyrrolo[3, | | |
| | 2-c]pyridin-2-yl)pyridin- | | |
| | 2-yl]phenyl}but-2-enamide | | |
| | trifluoroacetate | | |
| 274 | N-{2-[4-(4-oxo-4,5, | 359.1512 | 359.1503 |
| | 6,7-tetrahydro-1H-pyrrolo[3, | | |
| | 2-c]pyridin-2-yl)pyridin- | | |
| | 2-yl]phenyl}acrylamide | | |
| | trifluoroacetate | | |
| 275 | (2E)-N-{2-[4-(4-oxo-4,5, | 373.1659 | 373.1681 |
| | 6,7-tetrahydro-1H-pyrrolo[3, | | |
| | 2-c]pyridin-2-yl)pyridin- | | |
| | 2-yl]phenyl}but-2-enamide | | |
| | trifluoroacetate | | |
| 276 | 3-methyl-N-{2-[4-(4-oxo-4,5, | 387.1816 | 387.1819 |
| | 6,7-tetrahydro-1H-pyrrolo[3, | | |
| | 2-c]pyridin-2-yl)pyridin- | | |
| | 2-yl]phenyl}but-2-enamide | | |
| | trifluoroacetate | | |
| 277 | 3-methyl-N-{2-[4-(4-oxo-4,5, | 435.1816 | 435.1816 |
| | 6,7-tetrahydro-1H-pyrrolo[3, | | |
| | 2-c]pyridin-2-yl)pyridin- | | |
| | 2-yl]phenyl}but-2-enamide | | |
| | trifluoroacetate | | |
| 278 | (2E)-N-{4-[4-(4-oxo-4,5, | 435.1816 | 435.1841 |
| | 6,7-tetrahydro-1H-pyrrolo[3, | | |
| | 2-c]pyridin-2-yl)pyridin- | | |
| | 2-yl]phenyl}-3-phenylacrylamide | | |
| | trifluoroacetate | | |
| 279 | 3-bromo-N-{4-[4-(4-oxo-4,5, | 439.0764 | 439.0791 |
| | 6,7-tetrahydro-1H-pyrrolo[3, | | |
| | 2-c]pyridin-2-yl)pyridin- | | |
| | 2-yl]phenyl}propanamide | | |
| | trifluoroacetate | | |
| 280 | 2-methyl-N-{4-[4-(4-oxo-4,5, | 373.1666 | 373.1659 |
| | 6,7-tetrahydro-1H-pyrrolo[3, | | |
| | 2-c]pyridin-2-yl)pyridin- | | |
| | 2-yl]phenyl}acrylamide | | |
| | trifluoroacetate | | |
| 281 | (2E)-2-methyl-N-{4-[4-(4-oxo- | 387.1816 | 387.1851 |
| | 4,5,6,7-tetrahydro-1H-pyrrolo[3, | | |
| | 2-c]pyridin-2-yl)pyridin- | | |
| | 2-yl]phenyl}but-2-enamide | | |
| | trifluoroacetate | | |
| 282 | N-{4-[4-(4-oxo-4,5,6, | 387.1816 | 387.1806 |
| | 7-tetrahydro-1H-pyrrolo[3, | | |
| | 2-c]pyridin-2-yl)pyridin- | | |
| | 2-yl]phenyl}pent-4-enamide | | |
| | trifluoroacetate | | |
| 283 | (2Z)-4-oxo-4-({4-[4-(4-oxo- | 403.1401 | 403.1428 |
| | 4,5,6,7-tetrahydro-1H-pyrrolo[3, | | |
| | 2-c]pyridin-2-yl)pyridin- | | |
| | 2-yl]phenyl}amino)but-2- | | |
| | enoic acid trifluoroacetate | | |
| 284 | N-[2-(acryloylamino)ethyl]-N- | 457.1983 | 457.1968 |
| | [4-(4-oxo-4,5,6,7-tetrahydro- | | |
| | 1H-pyrrolo[3,2-c]pyridin- | | |
| | 2-yl)-2,3′-bipyridin- | | |
| | 6′-yl]acrylamide trifluoroacetate | | |
| 285 | tert-butyl 2-{acryloyl[4- | 503.2401 | 503.2414 |
| | (4-oxo-4,5,6,7-tetrahydro-1H- | | |
| | pyrrolo[3,2-c]pyridin- | | |
| | 2-yl)-2,3′-bipyridin- | | |
| | 6′-yl]amino}ethylcarbamate | | |
| | trifluoroacetate | | |
| 286 | N-(2-aminoethyl)-N-[4-(4-oxo- | 403.1877 | 403.1843 |
| | 4,5,6,7-tetrahydro-1H-pyrrolo[3, | | |
| | 2-c]pyridin-2-yl)-2,3′- | | |
| | bipyridin-6′-yl]acrylamide | | |
| | trifluoroacetate | | |
| 287 | 1-{4-[4-(4-oxo-4,5,6, | 385.1295 | 385.1264 |
| | 7-tetrahydro-1H-pyrrolo[3, | | |
| | 2-c]pyridin-2-yl)pyridin- | | |
| | 2-yl]phenyl}-1H-pyrrole-2, | | |
| | 5-dione trifluoroacetate | | |
| 288 | 1-{3-[4-(4-oxo-4,5,6, | 385.1295 | 385.127 |
| | 7-tetrahydro-1H-pyrrolo[3, | | |
| | 2-c]pyridin-2-yl)pyridin- | | |
| | 2-yl]phenyl}-1H-pyrrole-2, | | |
| | 5-dione trifluoroacetate | | |
| 289 | ethyl (2E)-4-oxo-4-({3- | 445.187 | 445.1864 |
| | [4-(4-oxo-4,5,6,7-tetrahydro- | | |
| | 1H-pyrrolo[3, | | |
| | 2-c]pyridin-2-yl)pyridin- | | |
| | 2-yl]benzyl}amino)but-2- | | |
| | enoate trifluoroacetate | | |
| 290 | (2Z)-4-oxo-4-({3-[4-(4-oxo- | 417.1557 | 417.1558 |
| | 4,5,6,7-tetrahydro-1H-pyrrolo[3, | | |
| | 2-c]pyridin-2-yl)pyridin- | | |
| | 2-yl]benzyl}amino)but-2- | | |
| | enoic acid trifluoroacetate | | |
| 291 | (2E)-4-oxo-4-({3-[4-(4-oxo- | 403.1401 | 403.1396 |
| | 4,5,6,7-tetrahydro-1H-pyrrolo[3, | | |
| | 2-c]pyridin-2-yl)pyridin- | | |
| | 2-yl]phenyl}amino)but-2- | | |
| | enoic acid trifluoroacetate | | |
| 292 | ethyl (2E)-4-oxo-4-({3-[4-(4- | 431.1714 | 431.1680 |
| | oxo-4,5,6,7-tetrahydro-1H-pyrrolo[3, | | |
| | 2-c]pyridin-2-yl)pyridin- | | |
| | 2-yl]phenyl}amino)but-2- | | |
| | enoate trifluoroacetate | | |

### EXAMPLE 293

This example illustrates the production of {5-[4-(4-oxo-4,5,6,7-tetrahydro-1H-pyrrolo[3,2-c]pyridin-2-yl)pyridin-2-yl]thien-2-yl}methyl thiocyanate trifluoroacetate. (Angew. Chem. Int. Ed. Engl. 1980,19,394 & TL 42(2001)8479-8481).

To (2-{2-[5-(hydroxymethyl)thien-2-yl]pyridin-4-yl}-1,5,6,7-tetrahydro-4H-pyrrolo[3,2-c]pyridin-4-one) (Example 39) (165 mg, 0.5 mmol) was added thionyl chloride (2 ml) with stirring, and kept stirred for 1 hour before it was N₂ stripped down to dryness. The residue was mixed with trimethylsilyl thioisocyanate (5 ml) in DMF (5 ml). To the resulting mixture was added tetrabutylammonium fluoride (1M in THF, 5 ml) and kept stirred overnight at ambient temperature. Then, it was concentrated a little, diluted with acetonitrile and water, and purified by prep-HPLC. (143 mg, yellow solid). ¹H NMR (400MHz, DMSO-d₆): δ 12(s, 1H), δ 9.18 (bs, 1H), δ 8.38 (d, 1H), δ 8.15 (s, 1H), δ 7.74 (d, 1H), δ 7.50 (dd, 1H), δ 7.20(s, 1H) δ 7.18 (s, 1H), δ 3.34 (t, 2H), δ 2.81 (m, 2H); m/z: 367.1 (M+H).

The examples in the table below were prepared using the general procedure as described for Example 293.

| Example | | Calculated | Found |
|---|---|---|---|
| No. | Compound Name(s) | (m + H) | m + H |
| 293 | {5-[4-(4-oxo-4,5,6, | 366.0609 | 367.1 |
| | 7-tetrahydro-1H-pyrrolo[3, | | |
| | 2-c]pyridin-2-yl)pyridin- | | |
| | 2-yl]thien-2-yl}methyl | | |
| | thiocyanate trifluoroacetate | | |
| 294 | 3-[4-(4-oxo-4,5,6,7-tetrahydro- | 360.1045 | 361.1 |
| | 1H-pyrrolo[3,2-c]pyridin- | | |
| | 2-yl)pyridin-2-yl]benzyl | | |
| | thiocyanate trifluoroacetate | | |
| 295 | 4-[4-(4-oxo-4,5,6,7-tetrahydro- | 360.1045 | 361.1 |
| | 1H-pyrrolo[3,2-c]pyridin- | | |
| | 2-yl)pyridin-2-yl]benzyl | | |
| | thiocyanate trifluoroacetate | | |

### EXAMPLE 296

Step1. (Preparation of tert-butyl 3-bromobenzylcarbamate). A mixture of 3-bromobenzylamine hydrochloride (5.25 g, 23.6 mmol) in tetrahydrofuran (100 ml) at 0° C. was treated with a 1 M solution of di-tert-butyl dicarbonate in tetrahydrofuran (24.8 ml) and stirred overnight. The resulting mixture was treated with ethyl acetate (200 ml) and washed with 1 M HCl (aq) and brine. The organic layer was dried over sodium sulfate, filtered and evaporated to give tert-butyl 3-bromobenzylcarbamate (5.3 g, 79%) as a white solid. m/z (M+H): 286

Step 2. (Preparation of tert-butyl 3-[4-(4-oxo-4,5,6,7-tetrahydro-1H-pyrrolo[3,2-c]pyridin-2-yl)pyridin-2-yl]benzylcarbamate).

tert-Butyl 3-bromobenzylcarbamate was converted to tert-butyl 3-(4,4,5,5-tetramethyl-1,3,2-dioxaborolan-2-yl)benzylcarbamate by the procedure described for Example 109. A mixture of 2-(2-chloropyridin-4-yl)-1,5,6,7-tetrahydro-4H-pyrrolo[3,2-c]pyridin-4-one (Example 1) (2.77 g, 11.2 mmol), tert-butyl 3-(4,4,5,5-tetramethyl-1,3,2-dioxaborolan-2-yl)benzylcarbamate (5.58 g, 16.8 mmol), tetrakis(triphenylphospine)palladium(0) (650 mg, 0.56 mmol), 2.0 M aqueous cesium carbonate (16.8 mL, 33.5 mmol), and dimethylformamide (35 mL) was stirred at 80° C. under nitrogen overnight. The reaction mixture was poured into 200 ml water and extracted with ethyl acetate (3×100 ml). The organic layers were treated with 50 ml of methanol and the ppt was collected by vacuum filtration to give 2.18 g of the title compound as a yellow solid. The filtrated was concentrated and 100 ml of ethyl acetate was added to give a second crop of tert-butyl 3-[4-(4-oxo-4,5,6,7-tetrahydro-1H-pyrrolo[3,2-c]pyridin-2-yl)pyridin-2-yl]benzylcarbamate as a grey solid (1.8 g, 85% yield). m/z (M+H): 419

Step 3. (Preparation of 2-{2-[3-(aminomethyl)phenyl]pyridin-4-yl}-1,5,6,7-tetrahydro-4H-pyrrolo[3,2-c]pyridin-4-one trifluoroacetate).

tert-Butyl 3-[4-(4-oxo-4,5,6,7-tetrahydro-1H-pyrrolo[3,2-c]pyridin-2-yl)pyridin-2-yl]benzylcarbamate (2.18 g, 5.22 mmol) was treated with a 4 M solution of HCl in Dioxane (20 ml, 80 mmol) and the mixture was stirred overnight. The solution was concentrated in vacuo to give 2-{2-[3-(aminomethyl)phenyl]pyridin-4-yl}-1,5,6,7-tetrahydro-4H-pyrrolo[3,2-c]pyridin-4-one hydrochloride (2.17 g, quantitative) as a yellow solid. A portion (0.25 g, 0.71 mmol) was purified by reverse-phase HPLC (5-30% acetonitrile/water/0.05% trifluoroacetic acid) to give 2-{2-[3-(aminomethyl)phenyl]pyridin-4-yl}-1,5,6,7-tetrahydro-4H-pyrrolo[3,2-c]pyridin-4-one trifluoroacetate (0.073 g, 24%) as a yellow solid. ¹H NMR (400 MHz, DMSO-d₆) δ 12.16 (s, 1H), 8.62 (d, J=5.5 Hz, 1H), 8.28 (bs, 5H), 8.16 (d, J=7.5 Hz, 1H), 7.69 (m, 1H), 7.62-7.56 (m, 2H), 7.27 (d, J=2.0 Hz, 1H), 7.11 (s, 1H), 4.16 (m, 2H), 3.44(t, J=6.0 Hz, 2H), 2.89 (t, J=6.9 Hz, 2H). HRMS calculated for C₁₉H₁₈N₄O (MH⁺) 319.1553, found 319.1570. Anal. calculated for C₁₉H₁₈N₄O.1.95 TFA.1.25 H₂O C, 48.83; H, 4.02; N, 9.95. Found: C, 48.80; H, 3.94; N, 10.04.

### EXAMPLE 297

This example illustrates the production of 2-chloro-N-{3-[4-(4-oxo-4,5,6,7-tetrahydro-1H-pyrrolo[3,2-c]pyridin-2-yl)pyridin-2-yl]benzyl}acetamide trifluoroacetate.

A solution of 2-{2-[3-(aminomethyl)phenyl]pyridin-4-yl}-1,5,6,7-tetrahydro-4H-pyrrolo[3,2-c]pyridin-4-one hydrochloride (0.3 g, 0.85 mmol) and N-methyl morpholine (0.28 ml, 2.54 mmol) in dimethylformamide (5 mL) at 0° C. was treated with chloroacetyl chloride (0.071 ml, 0.89 mmol) and the mixture was stirred overnight. The mixture was acidified with trifluoroacetic acid, and purified by reverse-phase HPLC (5-30% acetonitrile/water/0.05% trifluoroacetic acid) to give 2-chloro-N-{3-[4-(4-oxo-4,5,6,7-tetrahydro-1H-pyrrolo[3,2-c]pyridin-2-yl)pyridin-2-yl]benzyl}acetamide trifluoroacetate (0.122 g, 28%) as a yellow solid. ¹H NMR (400 MHz, DMSO-d₆) δ 12.30 (s, 1H), 8.84 (t, J=5.8 Hz, 1H), 8.63 (d, J=5.9 Hz, 1H), 8.32 (s, 1H), 7.99 (m, 2H), 7.83 (d, J=5.5 Hz, 1H), 7.57 (m, 1H), 7.45 (m, 2H), 7.18 (s, 1H), 4.43(d, J=5.8 Hz, 2H), 4.15(s, 2H), 3.44(t, J=6.7 Hz, 2H), 2.91 (t, J=6.7 Hz, 2H). HRMS calculated for C₂₁H₁₉ClN₄O₂ (MH⁺) 395.1269, found 395.1253. Anal. calculated for C₂₁H₁₉ClN₄O₂.1.10 TFA.1.05 H₂O C, 51.68; H, 4.15; N, 10.39. Found: C, 51.69; H, 4.14; N, 10.44.

### EXAMPLE 298

This example illustrates the production of N-{3-[4-(4-oxo-4,5,6,7-tetrahydro-1H-pyrrolo[3,2-c]pyridin-2-yl)pyridin-2-yl]benzyl}acrylamide trifluoroacetate.

The title compound was prepared from acryloyl chloride in the same manner as Example 297. ¹H NMR (400 MHz, DMSO-d₆) δ 12.30 (s, 1H), 8.70 (t, J=5.8 Hz, 1H), 8.62 (d, J=5.9 Hz, 1H), 8.32 (s, 1H), 8.02(s, 1H), 7.98 (d, J=7.9 Hz, 1H), 7.83 (d, J=5.4 Hz, 1H), 7.56 (t, J=7.6 Hz, 2H), 7.46 (d, J=8.6 Hz, 2H), 7.18 (s, 1H), 6.31(m, 1H), 6.14(m, 1H), 5.64(m, 1H), 4.47(d, J=5.9 Hz, 2H), 3.44(t, J=6.0 Hz, 2H), 2.91 (t, J=6.8 Hz, 2H). HRMS calculated for C₂₂H₂₀N₄O₂ (MH⁺) 373.1659, found 373.1656. Anal. calculated for C₂₂H₂₀N₄O₂.1.15 TFA.0.95 H₂O C, 56.06; H, 4.46; N, 10.76. Found: C, 56.00; H, 4.41; N, 10.79.

### EXAMPLE 299

This example illustrates the production of N-{3-[4-(4-oxo-4,5,6,7-tetrahydro-1H-pyrrolo[3,2-c]pyridin-2-yl) pyridin-2-yl]benzyl}but-2-ynamide trifluoroacetate.

A solution of 2-butynoic acid (0.085 g, 1.01 mmol) and N-methyl morpholine (0.28 ml, 2.54 mmol) in dimethylformamide (5 mL) at 0° C. was treated with TBTU (0.326 g, 1.01 mmol) and 2-{2-[3-(aminomethyl)phenyl]pyridin-4-yl}-1,5,6,7-tetrahydro-4H-pyrrolo[3,2-c]pyridin-4-one hydrochloride (0.30 g, 0.85 mmol) and the solution was stirred 30 minutes. The solution was acidified with trifluoroacetic acid, and purified by reverse-phase HPLC (5-25% acetonitrile/water/0.05% trifluoroacetic acid) to give N-{3-[4-(4-oxo-4,5,6,7-tetrahydro-1H-pyrrolo[3,2-c]pyridin-2-yl)pyridin-2-yl]benzyl}but-2-ynamide trifluoroacetate (97 mg, 30%) as a yellow solid. ¹H NMR (400 MHz, DMSO-d₆) δ 12.31 (s, 1H), 9.10 (t, J=6.1 Hz, 1H), 8.63 (d, J=5.9 Hz, 1H), 8.32 (s, 1H), 7.96 (m, 2H), 7.83 (d, J=5.3 Hz, 1H), 7.56 (t, J=7.9 Hz, 1H), 7.44 (m, 2H), 7.19 (s, 1H), 4.39(d, J=6.0 Hz, 2H), 3.44(t, J=6.7 Hz, 2H), 2.92 (t, J=6.8 Hz, 2H), 1.97 (s, 3H). HRMS calculated for C₂₃H₂₀N₄O₂ (MH⁺) 385.1659, found 385.1654. Anal. calculated for C₂₃H₂₀N₄O₂.1.10 TFA.0.25 H₂O C, 58.84; H, 4.23; N, 10.89. Found: C, 58.80; H, 4.13; N, 11.00.

### EXAMPLE 300

This example illustrates the production of (2E)-4-bromo-N-{3-[4-(4-oxo-4,5,6,7-tetrahydro-1H-pyrrolo[3,2-c]pyridin-2-yl)pyridin-2-yl]benzyl}but-2-enamide trifluoroacetate.

The title compound was prepared from 4-bromocrotonoic acid (Tsou, H., et. al. J. Med. Chem., 44.2719 (2001)) in the same manner as Example 299. ¹H NMR (400 MHz, DMSO-d₆) δ 12.31 (s, 1H), 8.78 (t, J=5.8 Hz, 1H), 8.62 (d, J=5.9 Hz, 1H), 8.33 (s, 1H), 8.02 (s, 1H), 7.98 (d, J=7.9 Hz, 1H), 7.83 (d, J=4.8 Hz, 1H), 7.56 (t, J=7.7 Hz, 1H), 7.45 (d, J=7.7 Hz, 2H), 7.19 (s, 1H), 6.74(m, 1H), 6.29(m, 1H), 4.47(d, J=5.8 Hz, 2H), 4.38(dd, J=1.4 Hz, 6.3 Hz, 2H) 3.44(t, J=6.0 Hz, 2H), 2.91 (t, J=6.8 Hz, 2H). HRMS calculated for C₂₃H₂₁BrN₄O₂ (MH⁺) 465.0921, found 465.0934. Anal. calculated for C₂₃H₂₁BrN₄O₂.0.55 TFA C, 54.82; H, 4.1 1; N, 10.61. Found: C, 54.85; H, 4.29; N, 10.33.

### EXAMPLE 301

This example illustrates the production of (2E)-4-(dimethylamino)-N-{3-[4-(4-oxo-4,5,6,7-tetrahydro-1H-pyrrolo[3,2-c]pyridin-2-yl)pyridin-2-yl]benzyl}but-2-enamide trifluoroacetate.

Step1. (Preparation of (2E)-4-bromo-N-{3-[4-(4-oxo-4,5,6,7-tetrahydro-1H-pyrrolo[3,2-c]pyridin-2-yl)pyridin-2-yl]benzyl}but-2-enamide). The title compound was prepared from(2E)-4-bromobut-2-enoyl chloride (Tsou, H., et. al. J. Med. Chem., 44.2719 (2001)) in the same manner as Example 297. The crude material was carried on without purification. m/z (M+H): 465

Step 2. (Preparation of (2E)-4-(dimethylamino)-N-{3-[4-(4-oxo-4,5,6,7-tetrahydro-1H-pyrrolo[3,2-c]pyridin-2-yl)pyridin-2-yl]benzyl}but-2-enamide trifluoroacetate). To a solution of dimethyl amine in tetrahydrofuran (2 M, 34 mmol) at 0° C. was added (2E)-4-bromo-N-{3-[4-(4-oxo-4,5,6,7-tetrahydro-1H-pyrrolo[3,2-c]pyridin-2-yl)pyridin-2-yl]benzyl}but-2-enamide (1.69 mmol). The mixture was stirred a total of 6 hrs and then stored overnight at −20° C. To the mixture was added 100 ml of water and 100 ml of ethyl acetate. The layers were separated and the aqueous layer was concentrated. The resulting residue was purified by reverse-phase HPLC (15-30% acetonitrile/water/0.05% trifluoroacetic acid) to give (2E)-4-(dimethylamino)-N-{3-[4-(4-oxo-4,5,6,7-tetrahydro-1H-pyrrolo[3,2-c]pyridin-2-yl)pyridin-2-yl]benzyl}but-2-enamide trifluoroacetate (0.46 g, 39%) as a yellow solid. ¹H NMR (400 MHz, DMSO-d₆) δ 12.20 (s, 1H), 9.82(s, 1H), 8.89 (t, J=5.8 Hz, 1H), 8.60 (d, J=5.5 Hz, 1H), 8.27 (s, 1H), 8.07 (s, 1H), 8.02 (d, J=7.6 Hz, 1H), 7.72 (d, J=4.3 Hz, 1H), 7.53 (t, J=7.7 Hz, 1H), 7.41 (d, J=7.5 Hz, 1H), 7.31 (s, 1H), 7.14(s, 1H), 6.64(m, 1H), 6.34(d, J=15.4 Hz, 1H), 4.48(d, J=5.6 Hz, 2H), 3.92(m, 2H) 3.44(m, 2H), 2.90 (t, J=6.8 Hz, 2H), 2.78(s, 6H). HRMS calculated for C₂₅H₂₇N₅O₂ (MH⁺) 430.2238, found 430.2224. Anal. calculated for C₂₅H₂₇N₅O₂.2.10 TFA.1.20 H₂O C, 50.79; H, 4.60; N, 10.14. Found: C, 50.80; H, 4.70; N, 10.02.

### EXAMPLE 302

This example illustrates the production of (2E)-4-Bromo-N-{4-[4-(4-oxo-4,5,6,7-tetrahydro-1H-pyrrolo[3,2-c]pyridin-2-yl)pyridin-2-yl]phenyl}but-2-enamide trifluoroacetate.

Step 1. Trimethylsilyl (2E)-4-bromobut-2-enoate was prepared by a literature method (M. Bellassoued, Synthesis, 1983; 745-746).

Step 2. (2E)-4-Bromo-N-{4-[4-(4-oxo-4,5,6,7-tetrahydro-1H-pyrrolo[3,2-c]pyridin-2-yl)pyridin-2-yl]phenyl}but-2-enamide trifluoroacetate was prepared by a literature method (A. Wissner, J. Med. Chem. 2003, 46, 49-63).

To 0.45 g (1.9 mmol) of trimethylsilyl 4-bromo-2-butenoate in 1 ml of CH2CL2 was added 1 ml of 2M of oxalyl chloride (2 mmol), followed by 1 drop of DMF. The solution was stirred for 2 h at room temperature. Solvent was evaporated. A solution of 2-[2-(3-aminophenyl)pyridin-4-yl]-1,5,6,7-tetrahydro-4H-pyrrolo[3,2-c]pyridin-4-one trifluoroacetate (0.5 g, 1.6 mmol) and N,N-diisopropylethylamine (0.47 g, 3.6 mmol) were dissolved in anhydrous THF (10 ml). The solution was cooled in an ice bath. To this reaction mixture was added acid chloride from above reaction in anhydrous THF (5 ml). The ice bathe was removed one hour later and reaction mixture was stirred at room temperature for another one hour. The reaction mixture was concentrated and purified by prep. rpHPLC, and lyophilized to give the yellow solid (80 mg, 10%). This compound was a mixture of bromo and chloro derivates. (M+H) 407.14 and 451.11.

### EXAMPLE 303

This example illustrates the production of (2E)-4-(Dimethylamino)-N-{3-[4-(4-oxo-4,5,6,7-tetrahydro-1H-pyrrolo[3,2-c]pyridin-2-yl)pyridin-2-yl]phenyl}but-2-enamide bis(trifluoroacetate).

(2E)-4-Bromo-N-{4-[4-(4-oxo-4,5,6,7-tetrahydro-1H-pyrrolo[3,2-c]pyridin-2-yl)pyridin-2-yl]phenyl}but-2-enamide trifluoroacetate (100 mg, 0.2 mmol) and dimethylamine (0.33 ml of 2M solution in THF) were mixed in THF (2 ml). The reaction mixture was stirred at room temperature for 3 hours. Additional dimethylamine (0.5 ml of 2M solution in THF) was added to the reaction mixture and stirred overnight. The reaction was purified by rpHPLC and lyophilized to give the yellow solid (10 mg, 8%). HRMS calculated for C₂₄H₂₅N₅O₂ (MH⁺) 416.2081, found 416.2091.

### EXAMPLE 304

This example illustrates the production of N-{3-[4-(4-oxo-4,5,6,7-tetrahydro-1H-pyrrolo[3,2-c]pyridin-2-yl)pyridin-2-yl]phenyl}acrylamide trifluoroacetate.

HRMS calculated for C₂₁H₁₈N₄O₂ (MH⁺) 359.1503, found 359.1505.

### EXAMPLE 305

This example illustrates the production of 2-Methyl-N-{3-[4-(4-oxo-4,5,6,7-tetrahydro-1H-pyrrolo[3,2-c]pyridin-2-yl)pyridin-2-yl]phenyl}acrylamide trifluoroacetate.

HRMS calculated for C₂₂H₂₀N₄O₂ (MH⁺) 373.1659, found 373.1650.

### EXAMPLE 306

This example illustrates the production of 3-Methyl-N-{3-[4-(4-oxo-4,5,6,7-tetrahydro-1H-pyrrolo[3,2-c]pyridin-2-yl)pyridin-2-yl]phenyl}but-2-enamide trifluoroacetate.

HRMS calculated for C₂₃H₂₂N₂O₄ (MH⁺) 387.1816, found 387.1838.

### EXAMPLE 307

This example illustrates the production of (2E)-N-{3-[4-(4-oxo-4,5,6,7-tetrahydro-1H-pyrrolo[3,2-c]pyridin-2-yl)pyridin-2-yl]phenyl}but-2-enamide trifluoroacetate.

HRMS calculated for C₂₂H₂₀N₄O₂ (MH⁺) 373.1695, found 373.1681.

### EXAMPLE 308

This example illustrates the production of N-{3-[4-(5-methacryloyl-4-oxo-4,5,6,7-tetrahydro-1H-pyrrolo[3,2-c]pyridin-2-yl)pyridin-2-yl]phenyl}-2-methylacrylamide trifluoroacetate.

HRMS calculated for C₂₆H₂₄N₄O₃ (MH⁺) 441.1921, found 441.1903.

### EXAMPLE 309

This example illustrates the production of 2-{2-[4-(oxiran-2-ylmethoxy)phenyl]pyridin-4-yl}-1,5,6,7-tetrahydro-4H-pyrrolo[3,2-c]pyridin-4-one.(J. Med. Chem.2002,45, 1348-1362).

A mixture of 2-[2-(4-hydroxyphenyl)pyridin-4-yl]-1,5,6,7-tetrahydro-4H-pyrrolo[3,2-c]pyridin-4-one (Example 74) (1.44 g, 4.7 mmol), bromomethyl-oxirane (1.94 ml, 23.5 mmol), and Cs₂CO₃ (1.53 g, 4.7 mmol) in acetonitrile (50 ml) was heated to reflux and kept overnight. The reaction mixture was filtered, the solid was washed with acetoniltrile. Filtrate was concentrated to dryness, then triturated with some acetonitrile and the yellow solid was collected. (930 mg) ¹HNMR (400 MHz, DMSO-d₆): δ 12(s, 1H), δ 8.5 (d, 1H), δ 8.16 (s, 1H), δ 8.12 (d, 2H), δ 7.51 (dd, 1H), δ 7.14(s, 1H) δ 7.10 (d, 2H), δ 7.04 (bs, 1H), δ 4.42 (d, 2H), δ 3.41 (m, 2H), δ 3.38 (m, 1H), δ 2.86 (d, 2H), δ 2.72 (d, 2H); m/z: 362.2(M+H).

### EXAMPLE 310

This example illustrates the production of 2-(2-{4-[3-(diethylamino)-2-hydroxypropoxy] phenyl}pyridin-4-yl)-1,5,6,7-tetrahydro-4H-pyrrolo[3,2-c]pyridin-4-one trifluoroacetate.

A mixture of 2-{2-[4-(oxiran-2-ylmethoxy)phenyl]pyridin-4-yl}-1,5,6,7-tetrahydro-4H-pyrrolo[3,2-c]pyridin-4-one (Example 309) (40 mg, 0.11 mmol) and diethylamine (0.5 ml) in anhydrous MeOH(1 ml) was stirred at 40° C. overnight, then purified by prep-HPLC to give the title compound as yellow oil (24 mg). ¹HNMR (400 MHz, DMSO-d₆): δ 12.40 (s, 1H), δ 9.2 (bs, 1H), δ 8.6 (d, 1H), δ 8.3 (s, 1H), δ 8.10 (d, 1H), δ 7.81 (dd, 1H), δ 7.41 (s, 1H), δ 7.20 (s, 1H), δ 7.18 (d, 2H), δ 4.32 (m, 1H), δ 4.11 (d, 4H), δ 3.32 (m, 6H), δ 2.97 (m, 2H), δ 1.26 (m, 6H); m/z: 435.3(M+H).

The compounds in the table below were prepared using the general procedure as described for Example 10.

| Example | | Calculated | Found |
|---|---|---|---|
| No. | Compound Name(s) | (m + H) | m + H |
| 309 | 2-{2-[4-(oxiran- | 361.1426 | 362.2 |
| | 2-ylmethoxy)phenyl]pyridin- | | |
| | 4-yl}-1,5,6,7-tetrahydro-4H- | | |
| | pyrrolo[3,2-c]pyridin-4-one | | |
| 311 | 2-{2-[4-(2-hydroxy-3-morpholin- | 448.2111 | 449.2 |
| | 4-ylpropoxy)phenyl]pyridin- | | |
| | 4-yl}-1,5,6,7-tetrahydro-4H- | | |
| | pyrrolo[3,2-c]pyridin-4- | | |
| | one trifluoroacetate | | |
| 310 | 2-(2-{4-[3-(diethylamino)-2- | 434.2318 | 435.3 |
| | hydroxypropoxy]phenyl}pyridin- | | |
| | 4-yl)-1,5,6,7-tetrahydro-4H- | | |
| | pyrrolo[3,2-c]pyridin-4- | | |
| | one trifluoroacetate | | |
| 312 | 2-{2-[4-(2-hydroxy-3-piperidin- | 446.2318 | 447.3 |
| | 1-ylpropoxy)phenyl]pyridin- | | |
| | 4-yl}-1,5,6,7-tetrahydro-4H- | | |
| | pyrrolo[3,2-c]pyridin-4- | | |
| | one trifluoroacetate | | |
| 313 | 2-(2-{4-[2-hydroxy- | 461.2427 | 462.3 |
| | 3-(4-methylpiperazin- | | |
| | 1-yl)propoxy]phenyl}pyridin- | | |
| | 4-yl)-1,5,6,7-tetrahydro-4H- | | |
| | pyrrolo[3,2-c]pyridin-4- | | |
| | one trifluoroacetate | | |
| 314 | 2-[2-(4-{2-hydroxy-3-[(2R)- | 515.2896 | 516.3 |
| | 2-(pyrrolidin-1-ylmethyl)pyrrolidin- | | |
| | 1-yl]propoxy}phenyl)pyridin- | | |
| | 4-yl]-1,5,6,7-tetrahydro-4H- | | |
| | pyrrolo[3,2-c]pyridin-4- | | |
| | one trifluoroacetate | | |
| 315 | 2-[2-(4-{3-[[2- | 463.2583 | 464.3 |
| | (dimethylamino)ethyl](methyl)amino]- | | |
| | 2-hydroxypropoxy}phenyl)pyridin- | | |
| | 4-yl]-1,5,6,7-tetrahydro-4H- | | |
| | pyrrolo[3,2-c]pyridin-4- | | |
| | one trifluoroacetate | | |

### EXAMPLE 316

This example illustrates the preparation of 2-methyl-N-{4-[4-(4-oxo-4,5,6,7-tetrahydro-1H-pyrrolo[3,2-c]pyridin-2-yl)pyridin-2-yl]phenyl}propanamide trifluoroacetate.

To the suspension of 2-[2-(4-aminophenyl)pyridin-4-yl]-1,5,6,7-tetrahydro-4H-pyrrolo[3,2-c]pyridin-4-one (180 mg, 0.43 mMol) in DMF (2.0 mL) at room temperature under nitrogen was added N-methyl morpholine (0.12 mL, 1.08 mMol). A clear solution formed after addition. To this resulting mixture was added isobutyryl chloride (59 mg, 0.56 mMol). After stirring at room temperature for overnight, the mixture was diluted with acetonitrile and water and then acidified to pH=1.0 with TFA. After filtration, the mother liquor was purified by reversed phase prep HPLC. The resulting solid was further purified by flash chromatography and eluted with a gradient of EtOAc (100 mL) to 10% MeOH/EtOAc (100 mL) to 15% MeOH/EtOAc (100 mL) and 20% MeOH/EtOAc (200 mL). Desired fractions were combined and concentrated and redissolved in a mixture of water/acetonitrile and freeze-dried to give a yellowish solid (100 mg). ¹H NMR (400 MHz, CD3OD) δ (ppm): 8.47 (d, J=6.4 Hz, 1H), 8.32 (d, J=2.0 Hz, 1H), 7.94 (dd, J=1.6, 6.4 Hz, 1H), 7.91 (s, 4H), 7.52 (s, 1H), 3.60 (t, J=7.2 Hz, 2H), 3.02 (t, J=6.8 Hz, 2H), 2.64-2.71 (m, 1H), 4.84 (d, J=6.8 Hz, 6H). Theoretical high resolution Mass (M+H) for C₂₂H₂₃N₄O₄: 375.1816; Found: 375.1801.

### EXAMPLE 317

This example illustrates the preparation of 2,2,2-trifluoro-N-{4-[4-(4-oxo-4,5,6,7-tetrahydro-1H-pyrrolo[3,2-c]pyridin-2-yl)pyridin-2-yl]phenyl}acetamide trifluoroacetate.

To a mixture of 2-[2-(4-aminophenyl)pyridin-4-yl]-1,5,6,7-tetrahydro-4H-pyrrolo[3,2-c]pyridin-4-one (180 mg, 0.43 mMol) and N-methyl morpholine (108.9 mg, 1.08 mMol) in DMF (2.0 mL) at 0° C. under nitrogen was added trifluoroacetic anhydride (117.4 mg, 0.56 mMol). The resulting mixture was stirred at 0° C. for an addition five minutes before it was warmed up to room temperature and stirred at that temperature for overnight. After that, the reaction mixture was acidified to pH=1.0 with TFA and purified by reversed phase prep HPLC to give desired product as a yellowish solid (140 mg). ¹H NMR (400 MHz, CD3OD) δ (ppm): 8.51 (d, J=6.4 Hz, 1H), 8.30 (d, J=2.0 Hz, 1H), 7.96-8.01 (m, 4H), 7.92 (dd, J=1.2, 5.6 Hz, 1H), 7.49 (s, 1H), 3.60 (t, J=7.2 Hz, 2H), 3.01 (t, J=7.2 Hz, 2H). Theoretical high resolution Mass (M+H) for C₂₀H₁₅F₃N₄O₂: 401.1220; Found: 401.1244.

### EXAMPLE 318

This example illustrates the preparation of N-{4-[4-(4-oxo-4,5,6,7-tetrahydro-1H-pyrrolo[3,2-c]pyridin-2-yl)pyridin-2-yl]phenyl}-2-phenylacetamide trifluoroacetate.

To the solution of phenyl acetic acid (110 mg, 0.81 mMol) in DMF (2.0 mL) at room temperature under nitrogen was added carbonyldiimidazole (131.3 mg, 0.81 mMol). 30 minutes later, 2-[2-(4-aminophenyl)pyridin-4-yl]-1,5,6,7-tetrahydro-4H-pyrrolo[3,2-c]pyridin-4-one(180 mg, 0.43 mMol) was added into the mixture followed by N-methylmorpholine (52.2 mg, 0.52 mMol). The resulting mixture was stirred at room temperature for overnight. After acidification to pH=1.0 by TFA, the mixture was purified by reversed phase prep HPLC to give desired product as a yellowish solid (130 mg). ¹H NMR (400 MHz, CD3OD) δ (ppm): 8.47 (d, J=6.4 Hz, 1H), 8.28 (br s, 1H), 7.87-7.93 (m, 5H), 7.49 (s, 1H), 7.23-7.36 (m, 5H), 3.73 (s, 2H), 3.60 (t, J=7.2 Hz, 2H), 3.01 (t, J=7.2 Hz, 2H). Theoretical high resolution Mass (M+H) for C₂₆H₂₃N₄O₂: 423.1816; Found: 423.1815.

### EXAMPLE 319

This example illustrates the preparation of N-{4-[4-(4-oxo-4,5,6,7-tetrahydro-1H-pyrrolo[3,2-c]pyridin-2-yl)pyridin-2-yl]phenyl}cyclohexanecarboxamide trifluoroacetate.

To the suspension of 2-[2-(4-aminophenyl)pyridin-4-yl]-1,5,6,7-tetrahydro-4H-pyrrolo[3,2-c]pyridin-4-one (180 mg, 0.43 mMol) in DMF (2.0 mL) at room temperature under nitrogen was added N-methyl morpholine (0.12 mL, 1.08 mMol). A clear solution formed after addition. To this resulting mixture was added cyclohexanecarbonyl chloride (75.6 mg, 0.52 mMol). After stirring at room temperature for overnight, the mixture was diluted with acetonitrile and water and then acidified to pH=1.0 with TFA. After filtration, the mother liquor was purified by reversed phase prep HPLC to give desired product as a yellowish solid (70 mg). ¹H NMR (400 MHz, CD3OD) δ (ppm):8.47 (d, J=6.8 Hz, 1H), 8.28 (d, J=1.6 Hz, 1H), 7.86-7.92 (m, 5H), 7.49 (s, 1H), 3.60 (t, J=6.8 Hz, 2H), 3.01(t, J=6.8 Hz, 2H), 2.37-2.44 (m, 1H), 1.82-1.91 (m, 4H), 1.71-1.74 (m, 1H), 1.49-1.58 (m, 2H), 1.22-1.42 (m, 3H). Theoretical high resolution Mass (M+H) for C₂₅H₂₇N₄O₂: 415.2129; Found: 415.2139.

### EXAMPLE 320

This example illustrates the preparation of N-{4-[4-(4-oxo-4,5,6,7-tetrahydro-1H-pyrrolo[3,2-c]pyridin-2-yl)pyridin-2-yl]phenyl}-3-phenylpropanamide trifluoroacetate.

To the suspension of 2-[2-(4-aminophenyl)pyridin-4-yl]-1,5,6,7-tetrahydro-4H-pyrrolo[3,2-c]pyridin-4-one (200 mg, 0.478 mMol) in DMF (2.0 mL) at room temperature under nitrogen was added N-methyl morpholine (0.13 mL, 1.196 mMol). A clear solution formed after addition. To this resulting mixture was added 3-phenylpropanoyl chloride (104.8 mg, 0.62 mMol). After stirring at room temperature for overnight, the mixture was diluted with acetonitrile and water and then acidified to pH=1.0 with TFA. After filtration, the mother liquor was purified by reversed phase prep HPLC to give desired product as a yellowish solid (94.4 mg). ¹H NMR (400 MHz, CD3OD) δ (ppm): 8.47 (d, J=6.4 Hz, 1H), 8.27 (d, J=2.0 Hz, 1H), 7.88-7.92 (m, 3H), 7.82-7.85 (m, 2H), 7.48 (s, 1H), 7.25-7.26 (m, 5H), 3.60 (t, J=6.8 Hz, 2H), 2.99-3.03 (m, 4H), 2.72 (t, J=8.0 Hz, 2H). Theoretical high resolution Mass (M+H) for C₂₇H₂₅N₄O₂: 437.1972; Found: 437.1987.

### EXAMPLE 321

This example illustrates the preparation of 2-(4-isopropylphenyl)-N-{4-[4-(4-oxo-4,5,6,7-tetrahydro-1H-pyrrolo[3,2-c]pyridin-2-yl)pyridin-2-yl]phenyl}acetamide trifluoroacetate.

To the solution of (4-isopropylphenyl)acetic acid (130 mg, 0.73 mMol) in DMF (2.0 mL) at room temperature under nitrogen was added carbonyldiimidazole (177.4 mg, 1.09 mMol). 30 minutes later, 2-[2-(4-aminophenyl)pyridin-4-yl]-1,5,6,7-tetrahydro-4H-pyrrolo[3,2-c]pyridin-4-one (254 mg, 0.43 mMol) was added into the mixture followed by N-methylmorpholine (52.2 mg, 0.61 mMol). The resulting mixture was stirred at room temperature for overnight. After acidification to pH=1.0 by TFA, the mixture was purified by reversed phase prep HPLC to give desired product as a yellowish solid (60 mg). ¹H NMR (400 MHz, CD3OD) δ (ppm): 8.46 (d, J=6.4 Hz, 1H), 8.26 (d, J=2.0 Hz, 1H), 7.86-7.92 (m, 5H), 7.47 (s, 1H), 7.25-7.28 (m, 2H), 7.18-7.21 (m, 2H), 3.68 (s, 2H), 3.60 (t, J=7.2 Hz, 2H), 3.01 (t, J=6.8 Hz, 2H), 2.82-2.91 (m, 1H), 1.22 (d, J=6.8 Hz, 6H). Theoretical high resolution Mass (M+H) for C₂₉H₂₉N₄O₂: 465.2285; Found: 465.2276.

### EXAMPLE 322

This example illustrates the preparation of 2-chloro-N-{4-[4-(4-oxo-4,5,6,7-tetrahydro-1H-pyrrolo[3,2-c]pyridin-2-yl)pyridin-2-yl]phenyl}acetamide trifluoroacetate.

To the suspension of 2-[2-(4-aminophenyl)pyridin-4-yl]-1,5,6,7-tetrahydro-4H-pyrrolo[3,2-c]pyridin-4-one (180 mg, 0.43 mMol) in DMF (2.0 mL) at room temperature under nitrogen was added N-methyl morpholine (0.12 mL, 1.08 mMol) followed by a-chloroacetyl chloride (72.8 mg, 0.645 mMol). After stirring at room temperature for overnight, the mixture was diluted with acetonitrile and water and then acidified to pH=1.0 with TFA. After filtration, the mother liquor was purified by reversed phase prep HPLC to give desired product as a yellowish solid (90 mg). ¹H NMR (400 MHz, CD3OD) δ (ppm): 8.47 (d, J=6.4 Hz, 1H), 8.27 (d, J=8.27 (d, J=2.0 Hz, 1H), 7.87-7.94 (m, 5H), 7.48 (s, 1H), 4.21 (s, 2H), 3.59 (t, J=6.8 Hz, 2H), 2.99 (t, J=6.8 Hz, 2H). Theoretical high resolution Mass (M+H) for C₂₀H₁₈ClN₄O₂: 381.1113; Found: 381.1135.

### EXAMPLE 323

This example illustrates the preparation of 2-bromo-N-{4-[4-(4-oxo-4,5,6,7-tetrahydro-1H-pyrrolo[3,2-c]pyridin-2-yl)pyridin-2-yl]phenyl}acetamide trifluoroacetate.

To the suspension of 2-[2-(4-aminophenyl)pyridin-4-yl]-1,5,6,7-tetrahydro-4H-pyrrolo[3,2-c]pyridin-4-one (130 mg, 0.31 mMol) in DMF (2.0 mL) at room temperature under nitrogen was added N-methyl morpholine (78.6 mg, 1.08 mMol) followed by a-chloroacetyl chloride (72.8 mg, 0.645 mMol). After stirring at room temperature for overnight, the mixture was diluted with acetonitrile and water and then acidified to pH=1.0 with TFA. After filtration, the mother liquor was purified by reversed phase prep HPLC to give desired product as a yellowish solid (90 mg). ¹H NMR (400 MHz, CD3OD) δ (ppm): 8.47 (d, J=6.4 Hz, 1H), 8.26 (d, J=2.0 Hz, 1H), 7.86-7.94 (m, 5H), 7.47 (s, 1H), 4.21&3.99 (s, 1H), 3.58 (t, J=6.8 Hz, 2H), 2.99 (t, J=7.2 Hz, 2H). Theoretical high resolution Mass (M+H) for C₂₀H₁₈BrN₄O₂: 425.0608; Found: 425.0647.

### EXAMPLE 324

This example illustrates the preparation of isobutyl 4-[4-(4-oxo-4,5,6,7-tetrahydro-1H-pyrrolo[3,2-c]pyridin-2-yl)pyridin-2-yl]phenylcarbamate trifluoroacetate.

To the suspension of 2-[2-(4-aminophenyl)pyridin-4-yl]-1,5,6,7-tetrahydro-4H-pyrrolo[3,2-c]pyridin-4-one (180 mg, 0.43 mMol) in DMF (2.0 mL) at room temperature under nitrogen was added N-methyl morpholine (108.9 mg, 1.08 mMol) followed by isobutylchloroformate (76.3 mg, 0.56 mMol). After stirring at room temperature for overnight, the mixture was diluted with acetonitrile and water and then acidified to pH=1.0 with TFA. After filtration, the mother liquor was purified by reversed phase prep HPLC to give desired product as a yellowish solid (120 mg). ¹H NMR (400 MHz, CD3OD) δ (ppm): 8.46 (d, J=6.4 Hz, 1H), 8.27 (d, J=1.6 Hz, 1H), 7.88-7.9 (m, 3H), 7.74-7.76 (m, 2H), 7.49 (s, 1H), 3.96 (d, J=6.4 Hz, 2H), 3.60 (t, J=7.2 Hz, 2H), 3.01 (t, J=7.2 Hz, 2H), 1.94-2.04 (m, 1H), 1.00 (d, J=6.8 Hz, 6H). Theoretical high resolution Mass (M+H) for C₂₃H₂₅N₄O₃: 405.1921; Found: 405.1912.

### EXAMPLE 325

This example illustrates the preparation of methyl 4-[4-(4-oxo-4,5,6,7-tetrahydro-1H-pyrrolo[3,2-c]pyridin-2-yl)pyridin-2-yl]phenylcarbamate trifluoroacetate.

To the suspension of 2-[2-(4-aminophenyl)pyridin-4-yl]-1,5,6,7-tetrahydro-4H-pyrrolo[3,2-c]pyridin-4-one (200 mg, 0.48 mMol) in DMF (2.0 mL) at room temperature under nitrogen was added N-methyl morpholine (72.6 mg, 0.72 mMol) followed by Methylchloroformate (76.3 mg, 0.56 mMol). After stirring at room temperature for overnight, the mixture was diluted with acetonitrile and water and then acidified to pH=1.0 with TFA. After filtration, the mother liquor was purified by reversed phase prep HPLC to give desired product as a yellowish solid (50 mg). ¹H NMR (400 MHz, CD3OD) δ (ppm): 8.46 (d, J=6.4 Hz, 1H), 8.27 (d, J=2.0 Hz, 1H), 7.88-7.91 (m, 3H), 7.73-7.76 (m, 2H), 7.49 (s, 1H), 3.77 (s, 3H), 3.60 (t, J=7.2 Hz, 2H), 3.01 (t, J=7.2 Hz, 2H). Theoretical high resolution Mass (M+H) for C₂₀H₁₉N₄O₃: 363.1452; Found: 363.1452.

### EXAMPLE 326

This example illustrates the preparation of benzyl 4-[4-(4-oxo-4,5,6,7-tetrahydro-1H-pyrrolo[3,2-c]pyridin-2-yl)pyridin-2-yl]phenylcarbamate trifluoroacetate.

To the suspension of 2-[2-(4-aminophenyl)pyridin-4-yl]-1,5,6,7-tetrahydro-4H-pyrrolo[3,2-c]pyridin-4-one (160 mg, 0.38 mMol) in DMF (2.0 mL) at room temperature under nitrogen was added N-methyl morpholine (58 mg, 0.57 mMol) followed by Benzylchloroformate (77.8 mg, 0.46 mMol). After stirring at room temperature for overnight, the mixture was diluted with acetonitrile and water and then acidified to pH=1.0 with TFA. After filtration, the mother liquor was purified by reversed phase prep HPLC to give desired product as a yellowish solid (75 mg). ¹H NMR (400 MHz, CD3OD) δ (ppm): 8.46 (d, J=6.4 Hz, 1H), 8.27 (d, J=2.0 Hz, 1H), 7.87-7.91 (m, 3H), 7.74-7.77 (m, 2H), 7.48 (s, 1H), 7.29-7.43 (m, 5H), 5.21 (s, 2H), 3.60 (t, J=7.2 Hz, 2H), 3.01 (t, J=7.2 Hz, 2H). Theoretical high resolution Mass (M+H) for C₂₆H₂₃N₄O₃: 439.1765; Found: 439.1748.

### EXAMPLE 327

This example illustrates the preparation of N-(2-hydroxyethyl)-N′-{4-[4-(4-oxo-4,5,6,7-tetrahydro-1H-pyrrolo[3,2-c]pyridin-2-yl)pyridin-2-yl]phenyl}urea trifluoroacetate.

To the suspension of 2-[2-(4-aminophenyl)pyridin-4-yl]-1,5,6,7-tetrahydro-4H-pyrrolo[3,2-c]pyridin-4-one (180 mg, 0.43 mMol) in DMF (2.0 mL) at room temperature under nitrogen was added N-methylmorpholine (65.2 mg, 0.65 mMol) and carbonyldiimidazole (90.64 mg, 0.56 mMol). After stirring at room temperature for overnight, 2-aminoethanol (39 mg, 0.65 mMol) was added and the resulting mixture was stirred for another six hours. Then it was diluted with acetonitrile and water and acidified to pH=1.0 with TFA, filtered and purified by reversed phase prep HPLC to give desired product as a yellowish solid (136.3 mg). ¹H NMR (400 MHz, CD3OD) δ (ppm): 8.44 (d, J=6.8 Hz, 1H), 8.28 (d, J=2.0 Hz, 1H), 7.90 (dd, J=2.0, 6.4 Hz, 1H), 7.85-7.87 (m, 2H), 7.67-7.71 (m, 2H), 7.50 (s, 1H), 3.64 (t, J=5.6 Hz, 2H), 3.60 (t, J=6.8 Hz, 2H), 3.338 (t, J=5.6 Hz, 2H), 3.02 (t, J=6.8 Hz, 2H). Theoretical high resolution Mass (M+H) for C₂₁H₂₂N₅O₃: 392.1717; Found: 392.1703.

### EXAMPLE 328

This example illustrates the preparation of N-{4-[4-(4-oxo-4,5,6,7-tetrahydro-1H-pyrrolo[3,2-c]pyridin-2-yl)pyridin-2-yl]phenyl}pyrrolidine-1-carboxamide trifluoroacetate

To the suspension of 2-[2-(4-aminophenyl)pyridin-4-yl]-1,5,6,7-tetrahydro-4H-pyrrolo[3,2-c]pyridin-4-one (180 mg, 0.43 mMol) in DMF (2.0 mL) at room temperature under nitrogen was added N-methylmorpholine (52.2 mg, 0.52 mMol) and carbonyldiimidazole (90.8 mg, 0.56 mMol). After stirring at room temperature for overnight, pyrrolidine (45.9 mg, 0.65 mMol) was added and the resulting mixture was stirred for another four hours. Then it was diluted with acetonitrile and water and acidified to pH=1.0 with TFA, filtered and purified by reversed phase prep HPLC to give desired product as a yellowish solid (90.0 mg). ¹H NMR (400 MHz, CD3OD) δ (ppm):8.44 (d, J=6.8 Hz, 1H), 8.29 (d, J=2.0 Hz, 1H), 7.91 (dd, J=1.6, 6.4 Hz, 1H), 7.85-7.88 (m, 2H), 7.77-7.80 (m, 2H),7.50 (s, 1H), 3.60 (t, J=6.8 Hz, 2H), 3.47-3.51 (m, 4H), 3.01 (t, J=7.2 Hz, 2H), 1.96-1.99 (m, 4H). Theoretical high resolution Mass (M+H) for C₂₃H₂₄N₅O₂: 402.1925; Found: 402.1939.

### EXAMPLE 329

This example illustrates the preparation of N-(2-morpholin-4-ylethyl)-N′-{4-[4-(4-oxo-4,5,6,7-tetrahydro-1H-pyrrolo[3,2-c]pyridin-2-yl)pyridin-2-yl]phenyl}urea bis(trifluoroacetate).

To the suspension of 2-[2-(4-aminophenyl)pyridin-4-yl]-1,5,6,7-tetrahydro-4H-pyrrolo[3,2-c]pyridin-4-one (180 mg, 0.43 mMol) in DMF (2.0 mL) at room temperature under nitrogen was added N-methylmorpholine (65.2 mg, 0.65 mMol) and carbonyldiimidazole (91 mg, 0.56 mMol). After stirring at room temperature for one hour, 2-morpholin-4-ylethanamine (84 mg, 0.65 mMol) was added and the resulting mixture was stirred for overnight. Then it was diluted with acetonitrile and water and acidified to pH=1.0 with TFA, filtered and purified by reversed phase prep HPLC to give desired product as a yellowish solid (60.0 mg). ¹H NMR (400 MHz, CD3OD) δ (ppm): 8.44 (d, J=5.6 Hz, 1H), 7.96 (d, J=0.8 Hz, 1H), 7.88 (d, J=8.8 Hz, 2H), 7.51 (d, J=8.8 Hz, 2H), 7.46 (dd, J=1.6, 5.2 Hz, 1H), 7.09 (s, 1H), 3.71 (t, J=4.4 Hz, 4H), 3.57 (t, J=6.8 Hz, 2H), 3.36 (t, J=6.4 Hz, 2H), 2.95 (t, J=7.2 Hz, 2H), 2.53-2.56 (m, 6H). Theoretical high resolution Mass (M+H) for C₂₅H₂₉N₆O₃: 461.2296; Found: 461.2293.

### EXAMPLE 330

This example illustrates the preparation of N-{4-[4-(4-oxo-4,5,6,7-tetrahydro-1H-pyrrolo[3,2-c]pyridin-2-yl)pyridin-2-yl]phenyl}morpholine-4-carboxamide trifluoroacetate.

To the suspension of 2-[2-(4-aminophenyl)pyridin-4-yl]-1,5,6,7-tetrahydro-4H-pyrrolo[3,2-c]pyridin-4-one (210 mg, 0.50 mMol) in DMF (2.0 mL) at room temperature under nitrogen was added N-methylmorpholine (75.9 mg, 0.75 mMol) and carbonyldiimidazole (105.9 mg, 0.65 mMol). After stirring at room temperature for one hour, morpholine (65.3 mg, 0.75 mMol) was added and the resulting mixture was stirred for overnight. Then it was diluted with acetonitrile and water and acidified to pH=1.0 with TFA, filtered and purified by reversed phase prep HPLC to give desired product as a yellowish solid (83.0 mg). ¹H NMR (400 MHz, CD3OD) δ (ppm): 8.45 (d, J=6.4 Hz, 1H), 8.30 (d, J=2.0 Hz, 1H), 7.92 (dd, J=2.0, 6.8 Hz, 1H), 7.86-7.89 (m, 2H), 7.71-7.74 (m, 2H), 7.52 (s, 1H), 3.72 (t, J=4.4 Hz, 4H), 3.60 (t, J=7.2 Hz, 2H), 3.54 (t, J=5.2 Hz, 4H), 3.02 (t, J=6.8 Hz, 2H). Theoretical high resolution Mass (M+H) for C₂₃H₂₄N₅O₃: 418.1874; Found: 418.1860.

### EXAMPLE 331

This example illustrates the preparation of 2-[2-(3-aminophenyl)pyridin-4-yl]-1,5,6,7-tetrahydro-4H-pyrrolo[3,2-c]pyridin-4-one trifluoroacetate.

The mixture of 3-aminophenylboronic acid monohydrate (1.51 g, 9.76 mMol), 2-(2-chloropyridin-4-yl)-1,5,6,7-tetrahydro-4H-pyrrolo[3,2-c]pyridin-4-one (2.01 g, 8.14 mMol), 36.0 mL cesium carbonate (2.0M aqueous solution) and PdCl₂(dppf)CH₂Cl₂ (417 mg, 0.57 mMol) in DMF (31 mL) was degassed and flushed with nitrogen three times. Then it was heated to 100° C. under nitrogen for overnight. After that, the mixture was cooled to room temperature and filtered. The mother liquor was acidified to pH=1.0 with TFA and extracted with EtOAc (100 mL). The mother liquor was the diluted with acetone till cloudy and then stood on bench for two hrs. After filtration, the filtrate was dried over vacuum to give desired product as a greenish solid (1.37 g). ¹H NMR (400 MHz, CD3OD) δ (ppm): 8.48 (d, J=6.4 Hz, 1H), 8.25 (d, J=1.6 Hz, 1H), 7.94 (dd, J=1.6, 6.4 Hz, 1H), 7.48 (s, 1H), 7.38-7.42 (m, 1H), 7.25-7.26 (m, 2H), 7.02-7.05 (m, 1H), 3.59 (t, J=6.8 Hz, 2H), 3.01 (t, J=6.8 Hz, 2H). Theoretical high resolution Mass (M+H) for C₁₈H₁₇N₄O: 305.1397; Found: 305.1417.

### EXAMPLE 332

This example illustrates the preparation of isobutyl 3-[4-(4-oxo-4,5,6,7-tetrahydro-1H-pyrrolo[3,2-c]pyridin-2-yl)pyridin-2-yl]phenylcarbamate trifluoroacetate.

To the suspension of 2-[2-(3-aminophenyl)pyridin-4-yl]-1,5,6,7-tetrahydro-4H-pyrrolo[3,2-c]pyridin-4-one (210 mg, 0.50 mMol) in DMF (2.0 mL) at room temperature under nitrogen was added N-methyl morpholine (127 mg, 1.25 mMol) followed by isobutylchloroformate (88.8 mg, 0.65 mMol). After stirring at room temperature overnight, the mixture was diluted with a mixture of acetonitrile and water and then acidified to pH=1.0 with TFA. After filtration, the mother liquor was purified by reversed phase prep HPLC to give desired product as a yellowish solid (178.2 mg). ¹H NMR (400 MHz, CD3OD) δ (ppm): 8.52 (d, J=6.4 Hz, 1H), 8.24 (d, J=1.6 Hz, 1H), 8.16 (s, 1H), 7.94 (dd, J=2.0, 6.4 Hz, 1H), 7.54-7.57 (m, 3H), 7.49 (s, 1H), 3.96 (d, J=6.4 Hz, 2H), 3.60 (t, J=6.8 Hz, 2H), 3.01 (t, J=6.8 Hz, 2H), 1.94-2.04 (m, 1H), 1.00 (d, J=6.8 Hz, 6H). Theoretical high resolution Mass (M+H) for C₂₃H₂₅N₄O₃: 405.1921; Found: 405.1932.

### EXAMPLE 333

This example illustrates the preparation of 2,2,2-trifluoro-N-{3-[4-(4-oxo-4,5,6,7-tetrahydro-1H-pyrrolo[3,2-c]pyridin-2-yl) pyridin-2-yl]phenyl}acetamide trifluoroacetate.

To a mixture of 2-[2-(3-aminophenyl)pyridin-4-yl]-1,5,6,7-tetrahydro-4H-pyrrolo[3,2-c]pyridin-4-one (220 mg, 0.526 mMol) and N-methyl morpholine (133 mg, 1.31 mMol) in DMF (2.0 mL) at 0° C. under nitrogen was added trifluoroacetic anhydride (165.7 mg, 0.79 mMol). The resulting mixture was stirred at 0° C. for an addition five minutes before it was warmed up to room temperature and stirred at that temperature for one hour. After that, the reaction mixture was acidified to pH=1.0 with TFA and purified by reversed phase prep HPLC to give desired product as a yellowish solid (94.4 mg). ¹H NMR (400 MHz, CD3OD) δ (ppm):8.55 (d, J=6.4 Hz, 1H), 8.34 (t, J=2.0 Hz, 1H), 8.23 (d, J=1.6 Hz, 1H), 7.91 (dd, J=1.6, 6.0 Hz, 1H), 7.77-7.80 (m, 2H), 7.66-7.68 (m, 1H), 7.45 (s, 1H), 3.60 (t, J=6.8 Hz, 2H), 3.01 (t, J=7.2 Hz, 2H). Theoretical high resolution Mass (M+H) for C₂₀H₁₆F₃N₄O₂: 401.1220; Found: 401.1249.

### EXAMPLE 334

This example illustrates the preparation of 2-chloro-N-{3-[4-(4-oxo-4,5,6,7-tetrahydro-1H-pyrrolo[3,2-c]pyridin-2-yl)pyridin-2-yl]phenyl}acetamide trifluoroacetate.

To a mixture of 2-[2-(3-aminophenyl)pyridin-4-yl]-1,5,6,7-tetrahydro-4H-pyrrolo[3,2-c]pyridin-4-one (180 mg, 0.43 mMol) and N-methyl morpholine (108.9 mg, 1.08 mMol) in DMF (2.0 mL) at ambient under nitrogen was added a-chloroacetylchloride (72.8 mg, 0.65 mMol). The resulting mixture was stirred at room temperature for two hours. After that, the reaction mixture was acidified to pH=1.0 with TFA and purified by reversed phase prep HPLC to give desired product as a yellowish solid (162.0 mg). ¹H NMR (400 MHz, CD3OD) δ (ppm): 8.51 (d, J=6.8 Hz, 1H), 8.34 (t, J=1.6 Hz, 1H), 8.23 (d, J=1.6 Hz, 1H), 7.92 (dd, J=2.0, 6.4 Hz, 1H), 7.57-7.68 (m, 3H), 7.47 (s, 1H), 4.22 (s, 2H), 3.58 (t, J=6.8 Hz, 2H), 2.99 (t, J=6.8 Hz, 2H). Theoretical high resolution Mass (M+H) for C₂₀H₁₈ClN₄O₂: 381.1113; Found: 381.1106.

### EXAMPLE 335

This example illustrates the preparation of 3-chloro-N-{3-[4-(4-oxo-4,5,6,7-tetrahydro-1H-pyrrolo[3,2-c]pyridin-2-yl)pyridin-2-yl]phenyl}propanamide trifluoroacetate.

To a mixture of 2-[2-(3-aminophenyl)pyridin-4-yl]-1,5,6,7-tetrahydro-4H-pyrrolo[3,2-c]pyridin-4-one (130 mg, 0.31 mMol) and N-methyl morpholine (78.6 mg, 0.78 mMol) in DMF (2.0 mL) at ambient under nitrogen was added 3-chloropropanoyl chloride (50.8 mg, 0.40 mMol). The resulting mixture was stirred at room temperature for one hour. After that, the reaction mixture was acidified to pH=1.0 with TFA and purified by reversed phase prep HPLC to give desired product as a yellowish solid (112.0 mg). ¹H NMR (400 MHz, CD3OD) δ (ppm): 8.52 (d, J=6.4 Hz, 1H), 8.39 (brs, 1H), 8.25 (d, J=2.0 Hz, 1H), 7.94 (dd, J=2.0, 6.4 Hz, 1H), 7.57-7.66 (m, 3H), 7.49 (s, 1H), 3.89 (t, J=6.0 Hz, 2H), 3.60 (t, J=7.2 Hz, 2H), 3.01 (t, J=6.8 Hz, 2H), 2.90 (t, J=6.4 Hz, 2H). Theoretical high resolution Mass (M+H) for C₂₁H₂₀ClN₄O₂: 395.1269; Found: 395.1267.

### EXAMPLE 336

This example illustrates the preparation of 2-bromo-N-{3-[4-(4-oxo-4,5,6,7-tetrahydro-1H-pyrrolo[3,2-c]pyridin-2-yl)pyridin-2-yl]phenyl}acetamide trifluoroacetate.

To a mixture of 2-[2-(3-aminophenyl)pyridin-4-yl]-1,5,6,7-tetrahydro-4H-pyrrolo[3,2-c]pyridin-4-one (100 mg, 0.24 mMol) and N-methyl morpholine (60.5 mg, 0.60 mMol) in DMF (2.0 mL) at ambient under nitrogen was added a-bromoacetylbromide (57.9 mg, 0.29 mMol). The resulting mixture was stirred at room temperature for one hour. After that, the reaction mixture was acidified to pH=1.0 with TFA and purified by reversed phase prep HPLC to give desired product as a yellowish solid. ¹H NMR (400 MHz, CD3OD) δ (ppm): 8.51 (d, J=6.0 Hz, 1H), 8.34 (t, J=2.0 Hz, 1H), 8.23 (d, J=1.6 Hz, 1H), 7.92 (dd, J=2.0, 6.8 Hz, 1H), 7.57-7.67 (m, 3H), 7.46 (s, 1H), 4.00 (s, 2H), 3.58 (t, J=7.2 Hz, 2H), 2.99 (t, J=6.8 Hz, 2H). Theoretical high resolution Mass (M+H) for C₂₀H₁₈BrN₄O₂: 425.0608; Found: 425.0625.

### EXAMPLE 337

This example illustrates the preparation of (2Z)-4-oxo-4-({3-[4-(4-oxo-4,5,6,7-tetrahydro-1H-pyrrolo[3,2-c]pyridin-2-yl)pyridin-2-yl]phenyl}amino) but-2-enoic acid trifluoroacetate.

To a mixture of 2-[2-(3-aminophenyl)pyridin-4-yl]-1,5,6,7-tetrahydro-4H-pyrrolo[3,2-c]pyridin-4-one (630 mg, 1.51 mMol) and N-methyl morpholine (381 mg, 3.77 mMol) in DMF (3.0 mL) at ambient under nitrogen was added maleic anhydride (222 mg, 2.26 mMol). The resulting mixture was stirred at room temperature for three hours. After that, the reaction mixture was diluted with a mixture of acetonitrile and water and then acidified to pH=1.0 with TFA. Precipitation formed and filtered. The filtrate was rinsed with water and acetonitrile and dried over vacuum line to give 480 mg desired product as a yellowish solid. The mother liquor was further purified by reversed phase prep HPLC to provide desired product as a yellowish solid in its TFA salt (85 mg). ¹H NMR (400 MHz, CD3OD) δ (ppm): 8.51 (d, J=6.4 Hz, 1H), 8.39 (t, J=2.0 Hz, 1H), 8.23 (d, J=2.0 Hz, 1H), 7.90 (dd, J=1.6, 6.0 Hz, 1H), 7.57-7.69 (m, 3H), 7.45 (s, 1H), 6.55 (d, J=12.4 Hz, 1H), 6.34 (d, J=12.4 Hz, 1H), 3.58 (t, J=7.2 Hz, 2H), 2.99 (t, J=7.2 Hz, 2H). Theoretical high resolution Mass (M+H) for C₂₂H₁₉N₄O₄: 403.1401; Found: 403.1396.

### EXAMPLE 338

This example illustrates the preparation of methyl (2Z)-4-oxo-4-({3-[4-(4-oxo-4,5,6,7-tetrahydro-1H-pyrrolo[3,2-c]pyridin-2-yl)pyridin-2-yl]phenyl}amino)but-2-enoate trifluoroacetate.

(2Z)-4-oxo-4-({3-[4-(4-oxo-4,5,6,7-tetrahydro-1H-pyrrolo[3,2-c]pyridin-2-yl)pyridin-2-yl]phenyl}amino)but-2-enoic acid (TFA salt) (210 mg) was treated with anhydrous methanol (2.0 mL) and 4N HCl/1,4-dioxane (3.0 mL) at room temperature for 3 hrs. After concentration, the residue was purified by reversed phase prep HPLC to give desired product as a yellowish solid (121.9 mg). ¹H NMR (400 MHz, CD3OD) δ (ppm): 8.50 (d, J=6.4 Hz, 1H), 8.37 (t, J=2.0 Hz, 1H), 7.91 (dd, J=2.0, 6.4 Hz, 1H), 7.56-7.66 (m, 3H), 7.46 (s, 1H), 6.55 (d, J=12.0 Hz, 1H), 6.34 (d, J=12.0 Hz, 1H), 3.58 (t, J=6.8 Hz, 2H), 2.99 (t, J=7.2 Hz, 2H). Theoretical high resolution Mass (M+H) for C₂₃H₂₁N₄O₄: 417.1557; Found: 417.1534.

### EXAMPLE 339

This example illustrates the preparation of 2-oxo-2-({3-[4-(4-oxo-4,5,6,7-tetrahydro-1H-pyrrolo[3,2-c]pyridin-2-yl)pyridin-²-yl]phenyl}amino)ethyl acetate trifluoroacetate.

To a mixture of 2-[2-(3-aminophenyl)pyridin-4-yl]-1,5,6,7-tetrahydro-4H-pyrrolo[3,2-c]pyridin-4-one (240 mg, 0.57 mMol) and N-methyl morpholine (145.2 mg, 1.43 mMol) in DMF (2.0 mL) at ambient under nitrogen was added acetoxyacetylchloride (101.9 mg, 0.75 mMol). The resulting mixture was stirred at room temperature for two hours. After that, the reaction mixture was acidified to pH=1.0 with TFA and purified by reversed phase prep HPLC to give desired product as a yellowish solid (217.8 mg). ¹H NMR (400 MHz, CD3OD) δ (ppm): 8.50 (d, J=6.4 Hz, 1H), 8.31 (t, J=1.6 Hz, 1H), 8.24 (d, J=2.0 Hz, 1H), 7.93 (dd, J=2.0, 6.4 Hz, 1H), 7.57-7.66 (m, 3H), 7.48 (s, 1H) 4.73 (s, 2H), 3.58 (t, J=7.2 Hz, 2H), 2.99 (t, J=6.8 Hz, 2H), 2.15 (s, 3H). Theoretical high resolution Mass (M+H) for C₂₂H₂₁N₄O₄: 405.1557; Found: 405.1550.

### EXAMPLE 340

This example illustrates the preparation of 2-hydroxy-N-{3-[4-(4-oxo-4,5,6,7-tetrahydro-1H-pyrrolo[3,2-c]pyridin-2-yl)pyridin-2-yl]phenyl}acetamide trifluoroacetate.

To a mixture of 2-[2-(3-aminophenyl)pyridin-4-yl]-1,5,6,7-tetrahydro-4H-pyrrolo[3,2-c]pyridin-4-one (1.55 g, 3.71 mMol) and N-methyl morpholine (937.7 mg, 9.27 mMol) in DMF (5.0 mL) at ambient under nitrogen was added acetoxyacetylchloride (658.5 mg, 4.82 mMol). The resulting mixture was stirred at room temperature for one hour and then treated with lithium hydroxide monohydrate (467.5 mg) and a mixture of water (10.0 mL) and ethanol (5.0 mL). The reaction went to completion one hour later. After that, the mixture was filter and the mother liquor was acidified to pH=1.0 with TFA and purified by reversed phase prep HPLC to give desired product as a yellowish solid (376.0 mg). ¹H NMR (400 MHz, CD3OD) δ (ppm): 8.53 (d, J=6.4 Hz, 1H), 8.33 (t, J=2.0 Hz, 1H), 8.27 (d, J=1.6 Hz, 1H), 7.76-7.79 (m, 1H), 7.67-7.69 (m, 1H), 7.59-7.63 (m, 1H), 7.49 (s, 1H), 4.16 (s, 2H), 3.60 (t, J=7.2 Hz, 2H), 3.01 (t, J=6.8 Hz, 2H). Theoretical high resolution Mass (M+H) for C₂₀H₁₉N₄O₃: 363.1452; Found: 363.1414.

### EXAMPLE 341

This example illustrates the preparation of 2-oxo-2-({3-[4-(4-oxo-4,5,6,7-tetrahydro-1H-pyrrolo[3,2-c]pyridin-2-yl)pyridin-2-yl]phenyl}amino)ethyl acrylate trifluoroacetate.

2-hydroxy-N-{3-[4-(4-oxo-4,5,6,7-tetrahydro-1H-pyrrolo[3,2-c]pyridin-2-yl)pyridin-2-yl]phenyl}acetamide (100 mg, 0.21 mMol) was dissolved in DMF (2.0 mL) at ambient under nitrogen. To this solution was added N-Methylmorpholine (63.6 mg, 0.62 mMol) followed by acryloyl chloride (19.0 mg, 0.32 mMol). The resulting mixture was stirred at room temperature for 60 hr before it was acidified to pH=1.0 with TFA and purified by reversed phase prep HPLC. Desired fractions were combined and freeze-dried to give desired product as a yellowish solid. ¹H NMR (400 MHz, CD3OD) δ (ppm): 8.49 (d, J=6.4 Hz, 1H), 8.31 (t, J=2.0 Hz, 1H), 8.23 (d, J=2.0 Hz, 1H), 7.91 (dd, J=1.6, 6.4 Hz, 1H), 7.57-7.67 (m, 3H), 7.46 (s, 1H), 6.48 (dd, J=1.2, 17.2 Hz, 1H), 6.29 (dd, J=10.4, 17.2 Hz, 1H), 5.97 (dd, J=1.6, 10.8 Hz, 1H), 3.58 (t, J=6.8 Hz, 2H), 2.99 (t, J=6.8 Hz, 2H). Theoretical high resolution Mass (M+H) for C₂₃H₂₁N₄O₄: 417.1557; Found: 417.1535.

### EXAMPLE 342

This example illustrates the preparation of 2-oxo-2-({3-[4-(4-oxo-4,5,6,7-tetrahydro-1H-pyrrolo[3,2-c]pyridin-2-yl)pyridin-2-yl]phenyl}amino)ethyl 4-(trifluoromethyl)benzoate trifluoroacetate.

2-hydroxy-N-{3-[4-(4-oxo-4,5,6,7-tetrahydro-1H-pyrrolo[3,2-c]pyridin-2-yl)pyridin-2-yl]phenyl}acetamide (140 mg, 0.29 mMol) was dissolved in DMF (2.0 mL) at ambient under nitrogen. To this solution was added N-Methylmorpholine (89.0 mg, 0.87 mMol) followed by 4-(trifluoromethyl)benzoyl chloride (90.7 mg, 0.44 mMol). The resulting mixture was stirred at room temperature for overnight before it was acidified to pH=1.0 with TFA and purified by reversed phase prep HPLC. Desired fractions were combined and freeze-dried to give desired product as a yellowish solid (100 mg). ¹H NMR (400 MHz, CD3OD) δ (ppm): 8.49 (d, J=6.4 Hz, 1H), 8.34 (brs, 1H), 8.26-8.28 (m, 3H), 7.95 (dd, J=2.0, 6.4 Hz, 1H), 7.81-7.83 (m, 2H), 7.59-7.67 (m, 3H), 7.49 (s, 1H), 5.03 (s, 2H), 3.57 (t, J=7.2 Hz, 2H), 2.98 (t, J=6.8 Hz, 2H). Theoretical high resolution Mass (M+H) for C₂₈H₂₂N₄O₄: 535.1588; Found: 535.1609.

### EXAMPLE 343

This example illustrates the preparation of 4-fluoro-N-{3-[4-(4-oxo-4,5,6,7-tetrahydro-1H-pyrrolo[3,2-c]pyridin-2-yl)pyridin-2-yl]phenyl}benzamide trifluoroacetate.

To a mixture of 2-[2-(3-aminophenyl)pyridin-4-yl]-1,5,6,7-tetrahydro-4H-pyrrolo[3,2-c]pyridin-4-one (240 mg, 0.57 mMol) and N-methyl morpholine (174.2 mg, 1.71 mMol) in DMF (2.0 mL) at ambient under nitrogen was added 4-fluorobenzoyl chloride (117.5 mg, 0.74 mMol). The resulting mixture was stirred at room temperature for 60 hrs. Then it was diluted with acetonitrile and water and acidified to pH=1.0 with TFA, filtered and purified by reversed phase prep HPLC to give desired product as a yellowish solid (120.0 mg). ¹H NMR (400 MHz, CD3OD) δ (ppm): 8.52(d, J=6.4 Hz, 1H), 8.43 (t, J=2.0 Hz, 1H), 8.27 (d, J=1.6 Hz, 1H), 8.01-8.04 (m, 2H), 7.93 (dd, J=2.0, 6.4 Hz, 1H), 7.77-7.79 (m, 1H), 7.67-7.70 (m, 1H), 7.60-7.64 (m, 1H), 7.48 (s, 1H), 7.22-7.27 (m, 2H), 3.58 (t, J=7.2 Hz, 2H), 2.99 (t, J=7.2 Hz, 2H). Theoretical high resolution Mass (M+H) for C₂₅H₂₀FN₄O₂: 427.1565; Found: 427.1566.

### EXAMPLE 344

This example illustrates the preparation of N-{3-[4-(4-oxo-4,5,6,7-tetrahydro-1H-pyrrolo[3,2-c]pyridin-2-yl)pyridin-2-yl]phenyl}-2-furamide trifluoroacetate.

To a mixture of 2-[2-(3-aminophenyl)pyridin-4-yl]-1,5,6,7-tetrahydro-4H-pyrrolo[3,2-c]pyridin-4-one (280 mg, 0.67 mMol) and N-methyl morpholine (203.3 mg, 2.01 mMol) in DMF (2.0 mL) at ambient under nitrogen was added 2-furoyl chloride (113.7 mg, 0.87 mMol). The resulting mixture was stirred at room temperature for 60 hrs. Then it was diluted with acetonitrile and water and acidified to pH=1.0 with TFA, filtered and purified by reversed phase prep HPLC to give desired product as a yellowish solid (156.0 mg). ¹H NMR (400 MHz, CD3OD) δ (ppm): 8.52 (d, J=6.4 Hz, 1H), 8.39 (t, J=2.0 Hz, 1H), 8.25 (d, J=1.6 Hz, 1H), 7.91 (dd, J=2.0, 6.8 Hz, 1H), 7.80-7.83 (m, 1H), 7.75-7.76 (m, 1H), 7.66-7.69 (m, 1H), 7.59-7.63 (m, 1H), 7.46 (s, 1H), 7.29-7.30 (m, 1H), 6.64-6.65 (m, 1H), 3.58 (t, J=7.2 Hz, 2H), 2.99 (t, J=7.2 Hz, 2H). Theoretical high resolution Mass (M+H) for C₂₃H₁₉N₄O₃: 399.1452; Found: 399.1443.

### EXAMPLE 345

This example illustrates the preparation of 2-(4-fluorophenyl)-N-{3-[4-(4-oxo-4,5,6,7-tetrahydro-1H-pyrrolo[3,2-c]pyridin-2-yl)pyridin-2-yl]phenyl}acetamide trifluoroacetate.

To a mixture of 2-[2-(3-aminophenyl)pyridin-4-yl]-1,5,6,7-tetrahydro-4H-pyrrolo[3,2-c]pyridin-4-one (220 mg, 0.53 mMol) and N-methyl morpholine (159.7 mg, 1.58 mMol) in DMF (2.0 mL) at ambient under nitrogen was added (4-fluorophenyl)acetyl chloride (136.2 mg, 0.79 mMol). The resulting mixture was stirred at room temperature for 60 hrs. Then it was diluted with acetonitrile and water and acidified to pH=1.0 with TFA, filtered and purified by reversed phase prep HPLC to give desired product as a yellowish solid (156.0 mg). ¹H NMR (400 MHz, CD3OD) δ (ppm): 8.48 (d, J=6.4 Hz, 1H), 8.34 (t, J=2.0 Hz, 1H), 8.21 (d, J=2.0 Hz, 1H), 7.91 (dd, J=2.0, 6.4 Hz, 1H), 7.55-7.63 (m, 3H), 7.46 (s, 1H), 7.34-7.38 (m, 2H), 7.01-7.07 (m, 2H), 3.70 (s, 2H), 3.57 (t, J=7.2 Hz, 2H), 2.98 (t, J=7.2 Hz, 2H). Theoretical high resolution Mass (M+H) for C₂₆H₂₂FN₄O₂: 441.1721; Found: 441.1698.

### EXAMPLE 346

This example illustrates the preparation of 2-(4-methoxyphenyl)-N-{3-[4-(4-oxo-4,5,6,7-tetrahydro-1H-pyrrolo[3,2-c]pyridin-2-yl)pyridin-2-yl]phenyl}acetamide trifluoroacetate.

To a mixture of 2-[2-(3-aminophenyl)pyridin-4-yl]-1,5,6,7-tetrahydro-4H-pyrrolo[3,2-c]pyridin-4-one (240 mg, 0.57 mMol) and N-methyl morpholine (174 mg, 1.72 mMol) in DMF (2.0 mL) at ambient under nitrogen was added (4-methoxyphenyl)acetyl chloride (157.8 mg, 0.85 mMol). The resulting mixture was stirred at room temperature for two hrs. Then it was diluted with acetonitrile and water and acidified to pH=1.0 with TFA, filtered and purified by reversed phase prep HPLC to give desired product as a yellowish solid (120.0 mg). ¹H NMR (400 MHz, CD3OD) δ (ppm): 8.48 (d, J=6.8 Hz, 1H), 8.34 (t, J=1.6 Hz, 1H), 8.21 (d, J=1.6 Hz, 1H), 7.91 (dd, J=2.0, 6.4 Hz, 1H), 7.54-7.63 (m, 3H), 7.46 (s, 1H), 7.25-7.27 (m, 2H), 6.85-6.88 (m, 2H) 3.75 (s, 3H), 3.64 (s, 2H), 3.58 (t, J=7.2 Hz, 2H), 2.98 (t, J=6.8 Hz, 2H). Theoretical high resolution Mass (M+H) for C₂₇H₂₅N₄O₃: 453.1921; Found: 453.1895.

### EXAMPLE 347

This example illustrates the preparation of 2-(3-methoxyphenyl)-N -{3-[4-(4-oxo-4,5,6,7-tetrahydro-1H-pyrrolo[3,2-c]pyridin-2-yl)pyridin-2-yl]phenyl}acetamide trifluoroacetate.

To a mixture of 2-[2-(3-aminophenyl)pyridin-4-yl]-1,5,6,7-tetrahydro-4H-pyrrolo[3,2-c]pyridin-4-one (240 mg, 0.57 mMol) and N-methyl morpholine (174 mg, 1.72 mMol) in DMF (2.0 mL) at ambient under nitrogen was added (3-methoxyphenyl)acetyl chloride (157.8 mg, 0.85 mMol). The resulting mixture was stirred at room temperature for two hrs. Then it was diluted with acetonitrile and water and acidified to pH=1.0 with TFA, filtered and purified by reversed phase prep HPLC to give desired product as a yellowish solid (150.0 mg). ¹H NMR (400 MHz, CD3OD) δ (ppm): 8.48 (d, J=6.4 Hz, 1H), 8.35 (t, J=2.0 Hz, 1H), 8.22 (d, J=1.6 Hz, 1H), 7.93 (dd, J=2.0, 6.4 Hz, 1H), 7.55-7.63 (m, 3H), 7.47 (s, 1H), 7.20-7.24 (m, 1H), 6.92-6.93 (m, 2H), 6.80-6.82 (m, 1H), 3.76 (s, 3H), 3.68 (s, 2H), 3.58 (t, J=7.2 Hz, 2H), 2.99 (t, J=7.2 Hz, 2H). Theoretical high resolution Mass (M+H) for C₂₇H₂₅N₄O₃: 453.1921; Found: 453.1931.

### EXAMPLE 348

This example illustrates the preparation of methyl 2-(methylamino)-5-[4-(4-oxo-4,5,6,7-tetrahydro-1H-pyrrolo[3,2-c]pyridin-2-yl)pyridin-2-yl]benzoate trifluoroacetate.

Step 1: To the solution of 2-amino-5-iodobenzoic acid (5.31 g, 20.19 mMol) in DMF (20.0 mL) at room temperature under nitrogen was added potassium carbonate (8.36 g, 60.57 mMol), followed by iodomethane (8.59 g, 60.57 mMol). The resulting mixture was stirred at ambient for overnight. Then it was diluted with EtOAc (150 mL) and washed successively with water (50 mL×3), and brine (50 mL), dried over sodium sulfate, filtered and concentrated. The resulting residue was purified by flash chromatography and eluted with a gradient of hexanes (200 mL) to 20% EtOAc/Hexanes (200 mL). Desired fractions were combined and concentrated to give a light yellowish oil of methyl 5-iodo-2-(methylamino)benzoate (2.78 g).

¹H NMR (400 MHz, CDCl3) δ (ppm): 8.14 (d, J=2.0 Hz, 1H), 7.57-7.59 (m, 1H), 6.50-6.52 (m, 1H), 3.83 (s, 3H), 2.87 (s, 3H). MS (M+H): 292.2.

Step 2: The mixture of Methyl 5-iodo-2-(methylamino)benzoate (2.78 g, 9.55 mMol), 4,4,4′,4′,5,5,5′,5′-octamethyl-2,2′-bi-1,3,2-dioxaborolane (2.67 g, 10.51 mMol), potassium acetate (2.81 g, 28.65 mMol) and PdCl₂(dppf).CH₂Cl₂ (209.6 mg, 0.29 mMol) in DMSO (36.0 mL) was degassed and flushed with nitrogen three times. Then it was heated to 80° C. under nitrogen. The progress of the reaction was monitored by TLC. After about three hours, the reaction went to completion. After cooling to room temperature, the mixture was diluted with EtOAc (200.0 mL) and washed with water (60 mL×3), brine (50.0 mL). The organic phase was dried over sodium sulfate, filtered and concentrated. The resulting residue was then filtered through a pad of silica gel and eluted with 30% EtOAc/Hexanes (300 mL). After concentration and drying over vacuum line, methyl 2-(methylamino)-5-(4,4,5,5-tetramethyl-1,3,2-dioxaborolan-2-yl)benzoate was obtained as a yellowish solid (2.62 g, 9.0 mMol). Then it was mixed with 2-(2-chloropyridin-4-yl)-1,5,6,7-tetrahydro-4H-pyrrolo[3,2-c]pyridin-4-one (1.85 g, 7.5 mMol) in DMF (35.0 mL). To this mixture was added 11.2 mL 2.0M aqueous sodium carbonate and PdCl₂(dppf)CH₂Cl₂ (384 mg, 0.525 mMol). The mixture was degassed and flushed with nitrogen before it was heated to 90° C. for overnight. After cooled to room temperature, the reaction was diluted with water and acetonitrile and then filtered. The mother liquor was acidified to pH=1.0 with TFA. Some of the acetonitrile was removed by rotatory evaporator. Then mixture was allowed to stand on top of the bench for about one hour. Precipitation formed and was filtered. MS (ES+) confirmed that solid to be the product. The mother liquor was purified by reversed phase prep HPLC to give desired product as a yellowish solid. Total: 1.6 g. ¹H NMR (400 MHz, CD3OD) δ (ppm): 8.52 (d, J=2.8 Hz, 1H), 8.34 (d, J=6.8 Hz, 1H), 8.21 (d, J=1.6 Hz, 1H), 7.98 (dd, J=2.4, 8.8 Hz, 1H), 7.81 (dd, J=2.0, 6.8 Hz, 1H), 7.47 (s, 1H), 6.98 (d, J=8.8 Hz, 1H), 3.91 (s, 3H), 3.59 (t, J=6.8 Hz, 2H), 3.00 (s, 3H), 3.00 (t, J=6.8 Hz, 2H). Theoretical high resolution Mass (M+H) for C₂₁ H₂, N₄O₃: 377.1608; Found: 377.1607.

### EXAMPLE 349

This example illustrates the preparation of 2-{2-[3-(hydroxymethyl)-4-(methylamino)phenyl]pyridin-4-yl}-1,5,6,7-tetrahydro-4H-pyrrolo[3,2-c]pyridin-4-one trifluoroacetate.

The suspension of methyl 2-(methylamino)-5-[4-(4-oxo-4,5,6,7-tetrahydro-1H-pyrrolo[3,2-c]pyridin-2-yl)pyridin-2-yl]benzoate (140 mg, 0.28 mMol) in THF (2.8 mL) was cooled to 0° C. under nitrogen. To this cold suspension was added a 1.0 Mether solution of lithium aluminum hydride (0.56 mL, 0.56 mMol). The resulting mixture was slowly warmed up to room temperature and stirred at that temperature for overnight. The reaction was slowly quenched with 1N aqueous HCl and stirred for ten minutes. Then it was purified by reversed phase prep HPLC to give desired product as a yellowish solid (40 mg). ¹H NMR (400 MHz, CD3OD) δ (ppm): 8.29 (d, J=6.8 Hz, 1H), 8.23 (d, J=2.0 Hz, 1H), 7.83 (dd, J=2.4, 8.8 Hz, 1H), 7.76-7.78 (m, 2H), 7.65 (s, 1H), 6.83 (d, J=8.4 Hz, 1H), 4.66 (s, 2H), 3.60 (t, J=7.2 Hz, 2H), 3.01 (t, J=6.8 Hz, 2H), 2.95 (s, 3H). Theoretical high resolution Mass (M+H) for C20H21 N4O2: 349.1659; Found: 349.1639.

### EXAMPLE 350

This example illustrates the preparation of 2-(4-amino-3-bromophenyl)pyridin-4-yl]-1,5,6,7-tetrahydro-4H-pyrrolo[3,2-c]pyridin-4-one trifluoroacetate. A vial was charged with 2-[2-(4-aminophenyl)pyridin-4-yl]-1,5,6,7-tetrahydro-4H-pyrrolo[3,2-c]pyridin-4-one trifluoroacetate (Example 78) (200.8 mg, 0.66 mmol) and dissolved in 2 ml DMSO. To the solution was added N-bromosuccinimide (660 μl) as a 1.0 M solution in DMSO. The solution was maintained at ambient temperature for 5 hours. Purification was accomplished by reversed phase HPLC yielding 66.8 mg of a yellow solid. ¹HNMR (400 MHz, CD₃OD) δ 8.34 (d, J=6.8 Hz, 1H), 8.23 (d, J=1.6 Hz, 1H), 8.07 (d, J=2.4 Hz, 1H), 7.83 (dd, J=6.8, 2.0 Hz, 1H), 7.71 (dd, J=8.4, 2.0 Hz, 1H), 7.49 (s, 1H), 6.98 (d, J=8.8 Hz, 1H), 3.60 (t, J=7.2 hz, 2H), 3.01 (t, J=6.8 Hz, 2H). m/z (M+H) 384.20.

### EXAMPLE 351

This example illustrates the preparation of {3-[4-(4-oxo-4,5,6,7-tetrahydro-1H-pyrrolo[3,2-c]pyridin-2-yl)pyridin-2-yl]phenoxy}acetic acid hydrochloride: The title compound was prepared by conversion of ethyl (3-bromophenoxy)acetate to its corresponding boronic ester (see Example 109 above) and coupled to 2-(2-chloropyridin-4-yl)-1,5,6,7-tetrahydro-4H-pyrrolo[3,2-c]pyridin-4-one (Example 1) (see Example 2 above). Hydrolysis of the ester occurred during the cross coupling reaction. Yield: 61%. ¹H NMR (300 MHz, DMSO-d₆): δ 13.0, s, 1H; 8.63, d, J=5.6, 1H; 8.62, s, 1H; 8.13, dd, J=6.5, 1.5; 1H; 7.74-7.68, m, 2H; 7.57, t, J=8.2, 1H; 7.22, dd, J=6.9, 1.5, 1H; 4.8, s, 2H; 3.44, t, J=6.4, 2H; 2.93, t, J=6.4, 2H. m/z: (M+H) 364. Calculated for C₂₀H₁₇N₃O₄ (M−H): 362.1141, found 362.1147.

### EXAMPLE 352

This example illustrates the preparation of 4-{3-[4-(4-oxo-4,5,6,7-tetrahydro-1H-pyrrolo[3,2-c]pyridin-2-yl)pyridin-2-yl]phenoxy}butanoic acid hydrochloride.

Step 1: Ethyl 4-(3-bromophenoxy)butanoate.

A solution of 3-bromophenol (1 mmol, 120 μL) in dimethylformamide (5 mL) was treated with postassium carbonate (138 mg, 2 mmol, 2.0 equiv.) and ethyl 4-bromobutanoate (172 μL, 1.2 mmol, 1.2 equiv.) and heated to 75° C. for 4 hours. Cooled to room temperature and diluted with ether. Washed with water (×6) dried over sodium sulfate and evaporated to provide the title compound as an oil (220 mg, 76%) ¹H NMR (400 MHz, CDCl₃): δ 7.16-7.04, m, 3H; 6.82, dq, J=8.1, 1.0, 1H; 4.16, q, J=7.3, 2H; 3.99, t, J=6.1, 2H; 2.50, t, J=7.5, 2H; 2.13, pent, J=6.8, 2H; 1.26, t, J=7.2, 3H. m/z: (M+H) 287.

Step 2: Ethyl 4-{3-[4-(4-oxo-4,5,6,7-tetrahydro-1H-pyrrolo[3,2-c]pyridin-2-yl)pyridin-2-yl]phenoxy}butanoate.

The title compound was prepared by conversion of Ethyl 4-(3-bromophenoxy)butanoate to its corresponding boronic ester (see Example 109 above) and coupled to 2-(2-chloropyridin-4-yl)-1,5,6,7-tetrahydro-4H-pyrrolo[3,2-c]pyridin-4-one (Example 1) (see Example 2 above). Yield: 56%. ¹H NMR (400 MHz, MeOD-d₄): δ 8.49, d, J=5.4, 1H; 8.0, m, 2H; 7.7-7.5, m, 3H; 7.4, t, J=8.3, 1H; 7.12, s, 1H; 7.0, d, J=8.0, 1H; 4.17-4.01, m, 4H; 3.59, t, J=7.0, 2H; 2.99 s, 1H; 2.96, t, J=7.0, 2H; 2.54, t, J=7.5, 2H; 2.11, pent., J=6.9, 2H; 1.24, t, J=7.5, 2H. m/z: (M+H) 420. Calculated for C₂₄H₂₅N₃O₄ (M+H): 420.1918, found 420.1902.

Step 3: A solution of Ethyl 4-{3-[4-(4-oxo-4,5,6,7-tetrahydro-1H-pyrrolo[3,2-c]pyridin-2-yl)pyridin-2-yl]phenoxy)butanoate (0.95 g, 2.26 mmol) in methanol (20 mL), 6 mL sodium hydroxide (2.5M) and water (25 mL) was heated to 90° C. for 2 hours. The solution was cooled, extracted with dichloromethane (2×20 mL). The pH was adjusted to 3 with concentrated aqueous hydrogen chloride. The title compound was collected as a bright yellow solid (883 mg, 100%). ¹H NMR (400 MHz, DMSO-d₆): 613.0, s, 1H; 8.62, d, J=6.4, 1H; 8.60, d, J=4.9, 1H; 8.12, dd, J=6.5, 1.6, 1H; 7.7-7.6, m, 3H; 7.55, t, J=8.3, 1H; 7.29, bs, 1H; 7.22, dd, J=8.2, 1.3, 1H; 4.15, t, J=6.4, 2H; 2.44, t, J=6.7, 2H; 2.93, t, J=6.7, 2H; 2.42, t, J=7.2, 2H; 1.99 pent., J=6.8, 2H. m/z: (M+H) 392. Calculated for C₂₂H₂₁N₃O₄ (M−H): 390.1454, found 390.1450.

### EXAMPLE 353

This example illustrates the preparation of 4-{4-[4-(4-oxo-4,5,6,7-tetrahydro-1H-pyrrolo[3,2-c]pyridin-2-yl)pyridin-2-yl]phenoxy}butanoic acid trifluoroacetate: The title compound was prepared by the method described for Example 352 using 4-bromophenol in lieu of 3-bromophenol. Calculated for C₂₂H₂₁N₃O₄ (M+H): 392.1605, found 392.1638.

### EXAMPLE 354

This example illustrates the preparation of ethyl (2E)-3-{3-[4-(4-oxo-4,5,6,7-tetrahydro-1H-pyrrolo[3,2-c]pyridin-2-yl)pyridin-2-yl]phenyl}acrylate. The title compound was prepared by conversion of ethyl (2E)-3-(3-bromophenyl)acrylate (Syn. Comm., 1995, 25, 2229) to its corresponding boronic ester (see Example 109 above) and coupled to 2-(2-chloropyridin-4-yl)-1,5,6,7-tetrahydro-4H-pyrrolo[3,2-c]pyridin-4-one (Example 1) (see Example 2 above). Yield: 13%. ¹H NMR (300 MHz, MeOD-d₄): δ 8.52, d, J=5.1, 1H; 8.21, s, 1H; 8.0, m, 2H; 7.77, d, J=10.2, 1H; 7.68, d, J=7.4, 1H;, 7.57-7.52, m, 2H; 7.13, s, 1H; 6.65, d, J=10.2, 1H; 4.26, q, J=7.0, 2H; 3.59, t, J=7.1, 1H; 2.97, t, J=7.1, wH; 1.34, t, J=7.0, 3H. m/z: (M+H) 388. Calculated for C₂₃H₂₁N₃O₃ (M+H): 388.1656, found 388.1667.

### EXAMPLE 355

This example illustrates the preparation of (2E)-3-{3-[4-(4-oxo-4,5,6,7-tetrahydro-1H-pyrrolo[3,2-c]pyridin-2-yl)pyridin-2-yl]phenyl}acrylic acid hydrochloride. The title compound was prepared from Ethyl (2E)-3-(3-[4-(4-oxo-4,5,6,7-tetrahydro-1H-pyrrolo[3,2-c]pyridin-2-yl)pyridin-2-yl]phenyl}acrylate (Example 354) by the procedure described for Example 352, step 3. ¹H NMR (300 MHz, DMSO-d₆): δ 13.35, bs, 1H: 8.77, s, 1H; 8.63, d, J=6.4, 1H; 8.56, s, 1H; 8.25, d, J=8, 1H; 8.19, dd, J=6.4, 1.5, 1H; 7.94, d, J=7.7, 1H; 7.7-7.6, m, 3H; 7.3, bs, 1H; 6.82, d, J=16.1, 1H; 3.43, t, J=6.7, 2H; 2.93, t, J=6.6, 2H. m/z: (M+H) 360. Calculated for C₂₁H₁₇N₃O₃(M+H): 360.1343, found 360.1344.

### EXAMPLE 356

This example illustrates the preparation of N,N-dimethyl-2-{3-[4-(4-oxo-4,5,6,7-tetrahydro-1H-pyrrolo[3,2-c]pyridin-2-yl)pyridin-2-yl]phenoxy}acetamide trifluoroacetate. {3-[4-(4-oxo-4,5,6,7-tetrahydro-1H-pyrrolo[3,2-c]pyridin-2-yl)pyridin-2-yl]phenoxy}acetic acid hydrochloride (Example 351) (100 mg, 0.28 mmol), N,N dimethylamine hydrochloride (68 mg, 0.84 mmol, 3 equiv) hydroxybenzotriazole (45 mg, 1.2 equiv.) and 1-(3-Dimethylaminopropyl)-3-ethylcarbodiimide hydrochloride (80 mg, 1.35 equiv.) and N-methylmorpholine (184 μL, 6 equiv.) were combined in dimethylfromamaide (5 mL) and heated to 40° C. for 1 hour. The product was isolated by reverse phase chromatography. The title compound was isolated as a yellow solid (32 mg, 22). ¹H NMR (400 MHz, MeOD-d₄): δ 8.52, d, J=6.1, 1H; 8.31, s, 1H; 7.98, dd, J=6.5, 1H; 7.59-7.50, m, 4H; 7.25, dd, J=7.4, 1.6, 1H; 4.96, s, 2H; 3.60, t, J=7.1, 2H; 3.11, s, 3H; 3.01, t, J=7.1, 2H; 2.98, s, 3H. m/z: (M+H) 391. Calculated for C₂₂H₂₂N₄O₃ (M+H): 391.1765, found 391.1764.

The following examples were prepared in a similar manner to Example 356:

| Example | | Calculated | Found |
|---|---|---|---|
| No. | Compound Name(s) | (m + H) | m + H |
| 357 | 2-{2-[4-(4-morpholin-4-yl-4- | 461.2183 | 461.2203 |
| | oxobutoxy)phenyl]pyridin-4-yl}- | | |
| | 1,5,6,7-tetrahydro-4H-pyrrolo[3, | | |
| | 2-c]pyridin-4-one trifluoroacetate | | |
| 358 | 2-[2-(4-{4-oxo-4-[(2R)-2- | 528.2969 | 528.2966 |
| | (pyrrolidin-1-ylmethyl)pyrrolidin- | | |
| | 1-yl]butoxy}phenyl)pyridin- | | |
| | 4-yl]-1,5,6,7-tetrahydro-4H- | | |
| | pyrrolo[3,2-c]pyridin-4-one | | |
| | trifluoroacetate | | |
| 359 | N-(2-morpholin-4-ylethyl)-4-{4- | 504.2605 | 504.2639 |
| | [4-(4-oxo-4,5,6,7-tetrahydro- | | |
| | 1H-pyrrolo[3,2-c]pyridin- | | |
| | 2-yl)pyridin-2-yl]phenoxy} | | |
| | butanamide trifluoroacetate | | |
| 360 | N,N-dimethyl-4-{4-[4-(4-oxo- | 419.2078 | 419.2101 |
| | 4,5,6,7-tetrahydro-1H-pyrrolo[3, | | |
| | 2-c]pyridin-2-yl)pyridin- | | |
| | 2-yl]phenoxy}butanamide | | |
| | trifluoroacetate | | |
| 361 | N-ethyl-4-{4-[4-(4-oxo-4,5, | 419.2078 | 419.2088 |
| | 6,7-tetrahydro-1H-pyrrolo[3, | | |
| | 2-c]pyridin-2-yl)pyridin- | | |
| | 2-yl]phenoxy}butanamide | | |
| | trifluoroacetate | | |
| 362 | N-ethyl-2-{3-[4-(4-oxo-4,5, | 391.1765 | 391.1784 |
| | 6,7-tetrahydro-1H-pyrrolo[3, | | |
| | 2-c]pyridin-2-yl)pyridin- | | |
| | 2-yl]phenoxy}acetamide | | |
| | trifluoroacetate | | |
| 363 | 2-[2-(3-{2-oxo-2-[(2R)-2- | 500.2656 | 500.2669 |
| | (pyrrolidin-1-ylmethyl)pyrrolidin- | | |
| | 1-yl]ethoxy}phenyl)pyridin- | | |
| | 4-yl]-1,5,6,7-tetrahydro-4H- | | |
| | pyrrolo[3,2-c]pyridin-4-one | | |
| | trifluoroacetate | | |
| 364 | N-(2-morpholin-4-ylethyl)-2-{3- | 476.2292 | 476.2308 |
| | [4-(4-oxo-4,5,6,7-tetrahydro-1H- | | |
| | pyrrolo[3,2-c]pyridin-2- | | |
| | yl)pyridin-2-yl]phenoxy} | | |
| | acetamide trifluoroacetate | | |
| 365 | N-ethyl-4-{3-[4-(4-oxo-4,5, | 419.2078 | 419.2058 |
| | 6,7-tetrahydro-1H-pyrrolo[3, | | |
| | 2-c]pyridin-2-yl)pyridin- | | |
| | 2-yl]phenoxy}butanamide | | |
| | trifluoroacetate | | |
| 366 | 2-[2-(3-{4-oxo-4-[(2S)-2- | 419.2078 | 419.2079 |
| | (pyrrolidin-1-ylmethyl)pyrrolidin- | | |
| | 1-yl]butoxy}phenyl)pyridin- | | |
| | 4-yl]-1,5,6,7-tetrahydro-4H- | | |
| | pyrrolo[3,2-c]pyridin-4-one | | |
| | trifluoroacetate | | |
| 367 | N-(2-morpholin-4-ylethyl)-4-{3- | 528.2969 | 528.2985 |
| | [4-(4-oxo-4,5,6,7-tetrahydro- | | |
| | 1H-pyrrolo[3,2-c]pyridin- | | |
| | 2-yl)pyridin-2-yl]phenoxy} | | |
| | butanamide trifluoroacetate | | |
| 355 | (2E)-3-{3-[4-(4-oxo-4,5,6, | 504.2605 | 504.261 |
| | 7-tetrahydro-1H-pyrrolo[3, | | |
| | 2-c]pyridin-2-yl)pyridin- | | |
| | 2-yl]phenyl}acrylic acid | | |
| | hydrochloride | | |
| 368 | (2E)-N,N-dimethyl-3-{3-[4-(4-oxo- | 387.1816 | 387.1805 |
| | 4,5,6,7-tetrahydro-1H-pyrrolo[3, | | |
| | 2-c]pyridin-2-yl)pyridin- | | |
| | 2-yl]phenyl}acrylamide | | |
| | trifluoroacetate | | |
| 369 | (2E)-N-ethyl-3-{3-[4-(4-oxo- | 387.1816 | 387.1802 |
| | 4,5,6,7-tetrahydro-1H-pyrrolo[3, | | |
| | 2-c]pyridin-2-yl)pyridin- | | |
| | 2-yl]phenyl}acrylamide | | |
| | trifluoroacetate | | |
| 370 | (2E)-N-(2-morpholin-4-ylethyl)- | 472.2343 | 472.2319 |
| | 3-{3-[4-(4-oxo-4,5,6, | | |
| | 7-tetrahydro-1H-pyrrolo[3, | | |
| | 2-c]pyridin-2-yl)pyridin- | | |
| | 2-yl]phenyl}acrylamide | | |
| | trifluoroacetate | | |
| 371 | 2-{2-[3-fluoro-4-(2-morpholin- | 450.1703 | 451.2 |
| | 4-yl-2-oxoethoxy)phenyl]pyridin- | | |
| | 4-yl}-1,5,6,7-tetrahydro-4H- | | |
| | pyrrolo[3,2-c]pyridin-4-one | | |
| | trifluoroacetate | | |
| 372 | 2-{2-[3-fluoro-4-(2-oxo-2- | 448.1911 | 449.2 |
| | piperidin-1-ylethoxy)phenyl]pyridin- | | |
| | 4-yl}-1,5,6,7-tetrahydro-4H- | | |
| | pyrrolo[3,2-c]pyridin-4-one | | |
| 373 | N,N-diethyl-2-{2-fluoro-4-[4-(4- | 436.1911 | 437.19 |
| | oxo-4,5,6,7-tetrahydro-1H-pyrrolo[3, | | |
| | 2-c]pyridin-2-yl)pyridin- | | |
| | 2-yl]phenoxy}acetamide | | |
| | trifluoroacetate | | |
| 374 | N-[2-(dimethylamino)ethyl]-2- | 465.2176 | 466.21 |
| | {2-fluoro-4-[4-(4-oxo-4,5,6, | | |
| | 7-tetrahydro-1H-pyrrolo[3, | | |
| | 2-c]pyridin-2-yl)pyridin- | | |
| | 2-yl]phenoxy}-N-methylacetamide | | |
| | trifluoroacetate | | |
| 375 | N-methyl-2-{4-[4-(4-oxo-4,5, | 376.1535 | 377.13 |
| | 6,7-tetrahydro-1H-pyrrolo[3, | | |
| | 2-c]pyridin-2-yl)pyridin- | | |
| | 2-yl]phenoxy}acetamide | | |
| | trifluoroacetate | | |
| 376 | 2-{2-[4-(2-morpholin-4-yl-2- | 432.1798 | 433.18 |
| | oxoethoxy)phenyl]pyridin-4-yl}- | | |
| | 1,5,6,7-tetrahydro-4H-pyrrolo[3, | | |
| | 2-c]pyridin-4-one trifluoroacetate | | |
| 377 | N-[2-(dimethylamino)ethyl]-N- | 447.227 | 448.23 |
| | methyl-2-{4-[4-(4-oxo-4,5, | | |
| | 6,7-tetrahydro-1H-pyrrolo[3, | | |
| | 2-c]pyridin-2-yl)pyridin- | | |
| | 2-yl]phenoxy}acetamide | | |
| | trifluoroacetate | | |
| 378 | 2-(2-{4-[2-(4-methylpiperazin- | 445.2114 | 446.23 |
| | 1-yl)-2-oxoethoxy]phenyl}pyridin- | | |
| | 4-yl)-1,5,6,7-tetrahydro-4H- | | |
| | pyrrolo[3,2-c]pyridin-4- | | |
| | one trifluoroacetate | | |

### EXAMPLE 379

This example illustrates the preparation of (2E)-N,N-dimethyl-3-{4-[4-(4-oxo-4,5,6,7-tetrahydro-1H-pyrrolo[3,2-c]pyridin-2-yl)pyridin-2-yl]phenyl}acrylamide trifluoroacetate.

A solution of (2E)-3-{4-[4-(4-oxo-4,5,6,7-tetrahydro-1H-pyrrolo[3,2-c]pyridin-2-yl)pyridin-2-yl]phenyl}prop-2-enoic acid trifluoroacetate (Example 92) (250 mg, 0.53 mmol), EDC (120 mg, 0.63 mmol), 1-hydroxybenzotriazole (85 mg, 0.63 mmol) and diisopropylethyl amine (0.26 mL, 1.6 mmol) in 5.0 mL of dimethylformamide was treated with dimethylamine 2.0 M solution in tetrahydrofuran (0.29 mL, 0.58 mmol) and stirred for 18 hours. The reaction was then filtered through a syringe filter (0.45 em), acidified with trifluoroacetic acid, purified by rpHPLC and lyophilized to give the title compound as a yellow solid (155 mg, 0.31 mmol, 58%). %). ¹H NMR (300 MHz, DMSO-d₆) δ 12.30 (s, 1H), 8.63 (d, J=5.8 Hz, 1H), 8.39 (s, 1H), 8.14 (d, J=8.4 Hz, 2H), 7.93 (d, J=8.3 Hz, 1H), 7.83 (d, J=4.8 Hz, 1H), 7.54 (d, J=15.5 Hz, 1H), 7.46 (s, 1H), 7.35 (d, J=15.5 Hz, 1H), 7.19 (s, 1H), 3.43 (t, J=6.7 Hz, 2H), 3.19 (s, 3H), 2.98-2.88 (m, 5H). HRMS calculated for C₂₃H₂₂N₄O₂ (MH⁺) 387.1816, found 387.1852. Anal. calculated for C₂₃H₂₂N₄O₂.1.1 TFA.2.7 H₂O C, 54.00; H, 5.07; N, 10.03. Found: C, 54.00; H, 5.08; N, 10.03.

The following examples were prepared by the same method.

| Example | | Calculated | Found |
|---|---|---|---|
| No. | Compound Name(s) | (m + H) | (m + H) |
| 380 | (2E)-N-ethyl-3-{4-[4-(4-oxo- | 387.1816 | 387.1828 |
| | 4,5,6,7-tetrahydro-1H-pyrrolo[3, | | |
| | 2-c]pyridin-2-yl)pyridin- | | |
| | 2-yl]phenyl}acrylamide | | |
| | trifluoroacetate | | |
| 381 | 2-(2-{4-[(1E)-3-morpholin- | 429.1921 | 429.1933 |
| | 4-yl-3-oxoprop-1-enyl]phenyl} | | |
| | pyridin-4-yl)-1,5,6,7-tetrahydro-4H- | | |
| | pyrrolo[3,2-c]pyridin-4-one | | |
| | trifluoroacetate | | |
| 382 | 2-(2-{4-[(1E)-3-oxo-3-pyrrolidin- | 413.1972 | 413.1982 |
| | 1-ylprop-1-enyl]phenyl}pyridin- | | |
| | 4-yl)-1,5,6,7-tetrahydro-4H- | | |
| | pyrrolo[3,2-c]pyridin-4- | | |
| | one trifluoroacetate | | |
| 383 | 2-[2-(4-{(1E)-3-oxo-3-[(2R)-2- | 496.2707 | 496.2702 |
| | (pyrrolidin-1-ylmethyl)pyrrolidin- | | |
| | 1-yl]prop-1-enyl}phenyl)pyridin- | | |
| | 4-yl]-1,5,6,7-tetrahydro-4H- | | |
| | pyrrolo[3,2-c]pyridin-4-one | | |
| | trifluoroacetate | | |

### EXAMPLE 384

This example illustrates the preparation of 2-{2-[4-(5,6-dihydro-1,4-oxathiin-2-yl)phenyl]pyridin-4-yl}-1,5,6,7-tetrahydro-4H-pyrrolo[3,2-c]pyridin-4-one trifluoroacetate.

Step 1: 2-[(2-hydroxyethyl)thio]-1-[4-(4,4,5,5-tetramethyl-1,3,2-dioxaborolan-2-yl)phenyl]ethanone was prepared using the general method described for Example 109 from 2-mercaproethanol and 2-4′-Dibromoacetophenone.

Step 2: A mixture of 2-(2-chloropyridin-4-yl)-1,5,6,7-tetrahydro-4H-pyrrolo[3,2-c]pyridin-4-one (359.3 mg, 1.45 mmol), 2-[(2-hydroxyethyl)thio]-1-[4-(4,4,5,5-tetramethyl-1,3,2-dioxaborolan-2-yl)phenyl]ethanone (514.0 mg, 1.60 mmol) and cesium carbonate, 2.0 M solution (2.2 ml, 4.4 mmol) in DMF (15 ml) was purged with nitrogen for 20 minutes. To this mixture was added tetrokistriphenylhosphinepalladium (134.0 mg, 0.12 mmol) and resultant mixture heated to 80° C. overnight. The mixtue was cooled to ambient temperature and filtered through a cake of Celite. Purification was accomplished by reversed phase HPLC yielding 28.8 mg of a orange solid. ¹HNMR (400 MHz, CD₃OD) δ 8.47 (d, J=6.4 Hz, 1H), 8.31 (s, 1H), 7.94 (d, J=6.4 Hz, 1H), 7.87 (d, J=8.8 Hz, 2H), 7.76 (d, J=8.8 Hz, 2H), 7.51 (s, 1H), 6.22 (s, 1H), 4.46 (m, 2H), 3.59 (t, J=6.8 Hz, 2H), 3.10 (m, 2H), 3.00 (t, J=7.2 Hz, 2H). m/z (M+H) 390.23.

### EXAMPLE 385

This example illustrates the preparation of N-butyl-4-[4-(4-oxo-4,5,6,7-tetrahydro-1H-pyrrolo[3,2-c]pyridin-2-yl)pyridin-2-yl]benzamide trifluoroacetate.

A solution of 4-[4-(4-oxo-4,5,6,7-tetrahydro-1H-pyrrolo[3,2-c]pyridin-2-yl)pyridin-2-yl]benzoic acid trifluoroacetate (200 mg, 0.77 mmol), EDCl (164 mg, 0.86 mmol) and HOBt (115 mg, 0.86 mmol) in 5.0 mL of dimethylformamide was treated with diisopropylethyl amine (0.38 mL, 2.3 mmol) followed by n-butyl amine (0.90 mL, 0.92 mmol) and stirred for 24 hours. The reaction contents were filtered through a syringe filter (0.45 μm) and purified by rpHPLC, and lyophilized to give the title compound as a yellow solid (90 mg, 0.17 mmol, 22%). ¹H NMR (300 MHz, DMSO-d₆) δ 12.24 (s, 1H), 8.63 (d, J=5.6 Hz, 1H), 8.58 (t, J=5.6 Hz, 1H), 8.37 (s, 1H), 8.20 (d, J=8.5 Hz, 2H), 8.01 (d, J=8.5 Hz, 2H), 7.80 (d, J=5.6 Hz, 1H), 7.40 (s, 1H), 7.16 (s, 1H), 3.43 (t, J=6.7 Hz, 2H), 3.28 (m, 2H), 2.90 (t, J=6.6 Hz, 2H), 1.53 (quintet, 5H), 1.34 (sextet, 2H), 0.91 (t, J=7.2 Hz, 3H). HRMS calculated for C₂₃H₂₄N₄O₂ (MH⁺) 389.1972, found 389.1948. Anal. calculated for C₂₃H₂₄N₄O₂.1.0 TFA.1.2 H₂O C, 57.29; H, 5.26; N, 10.68. Found: C, 57.17; H, 5.13; N, 10.75.

The following examples were prepared in the same manner.

| Example | | Calculated | Found |
|---|---|---|---|
| No. | Compound Name(s) | (m + H) | (m + H) |
| 386 | N-benzyl-N-methyl-4-[4-(4- | 437.1972 | 437.1935 |
| | oxo-4,5,6,7-tetrahydro-1H- | | |
| | pyrrolo[3,2-c]pyridin- | | |
| | 2-yl)pyridin-2-yl]benzamide | | |
| 387 | N-(2-methoxyethyl)-4-[4-(4- | 391.1765 | 391.1762 |
| | oxo-4,5,6,7-tetrahydro-1H- | | |
| | pyrrolo[3,2-c]pyridin- | | |
| | 2-yl)pyridin-2-yl]benzamide | | |
| | trifluoroacetate | | |
| 388 | N-cyclohexyl-N-methyl-4-[4-(4- | 429.2285 | 429.2251 |
| | oxo-4,5,6,7-tetrahydro-1H- | | |
| | pyrrolo[3,2-c]pyridin- | | |
| | 2-yl)pyridin-2-yl]benzamide | | |
| | trifluoroacetate | | |
| 389 | 4-[4-(4-oxo-4,5,6,7-tetrahydro- | 429.138 | 429.1376 |
| | 1H-pyrrolo[3,2-c]pyridin- | | |
| | 2-yl)pyridin-2-yl]-N-(thien- | | |
| | 2-ylmethyl)benzamide | | |
| | trifluoroacetate | | |
| 390 | 4-[4-(4-oxo-4,5,6,7-tetrahydro- | 437.1972 | 437.1991 |
| | 1H-pyrrolo[3,2-c]pyridin- | | |
| | 2-yl)pyridin-2-yl]-N-(2- | | |
| | phenylethyl)benzamide | | |
| | trifluoroacetate | | |
| 391 | 4-[4-(4-oxo-4,5,6,7-tetrahydro- | 409.1659 | 409.17 |
| | 1H-pyrrolo[3,2-c]pyridin- | | |
| | 2-yl)pyridin-2-yl]-N- | | |
| | phenylbenzamide trifluoroacetate | | |
| 392 | 4-[4-(4-oxo-4,5,6,7-tetrahydro- | 415.1376 | 415.1364 |
| | 1H-pyrrolo[3,2-c]pyridin- | | |
| | 2-yl)pyridin-2-yl]-N-(2,2, | | |
| | 2-trifluoroethyl)benzamide | | |
| | trifluoroacetate | | |

### EXAMPLE 393

This example illustrates the preparation of 2-{2-[4-(aminoacetyl)phenyl]pyridin-4-yl}-1,5,6,7-tetrahydro-4H-pyrrolo[3,2-c]pyridin-4-one bis(trifluoroacetate).

2-{2-[4-(N-Tert-butoxycarbonyl-aminoacetyl)phenyl]pyridin-4-yl}-1,5,6,7-tetrahydro-4H-pyrrolo[3,2-c]pyridin-4-one (Example 133, 489 mg, 1.10 mmol) was dissolved in trifluoroacetic acid (10 mL). After stirring for one hour at ambient temperature, the solvent was evaporated under a stream of nitrogen, and the resultant residue was purified by reverse-phase HPLC (acetonitrile/water/0.05% trifluoroacetic acid) to give 2-{2-[4-(aminoacetyl)phenyl]pyridin-4-yl}-1,5,6,7-tetrahydro-4H-pyrrolo[3,2-c]pyridin-4-one bis(trifluoroacetate) as a yellow solid (395 mg, 0.688 mmol, 63% yield). ¹H NMR (300 MHz, DMSO-d₆) δ 12.16 (s, 1H), 8.64 (d, J=5.4, 1H), 8.39 (s, 1H), 8.37 (d, J=8.5, 2H), 8.29 (bs, 2H), 8.16 (d, J=8.5, 2H), 7.72 (d, J=4.1, 1H), 7.30 (s, 1H), 7.11 (s, 1H), 4.68 (d, J=4.4, 2H), 3.43 (t, J=6.4, 2H), 2.89 (t, J=6.7, 2H). HRMS calculated for C₂₀H₁₉N₄O₂ (MH⁺) 347.1503, found 347.1489.

### EXAMPLE 394

This example illustrates the preparation of 1-methyl-2-(2-quinolin-3-ylpyridin-4-yl)-1,5,6,7-tetrahydro-4H-pyrrolo[3,2-c]pyridin-4-one.

A solution of 2-(2-quinolin-3-ylpyridin-4-yl)-1,5,6,7-tetrahydro-4H-pyrrolo[3,2-c]pyridin-4-one trifluoroacetate (150 mg, 0.441 mmol) in dimethylformamide (5 mL) was cooled to 0° C. and treated with sodium hydride (60% dispersion in mineral oil, 17.6 mg, 0.441 mmol). The resultant mixture was allowed to warm to room temperature over 45 min. Methyl iodide (0.027 mL, 0.441 mmol) was added, and the reaction mixture was allowed to stir overnight at room temperature. The reaction was quenched with saturated ammonium chloride, diluted with water, and filtered. The precipitate was dissolved in DMF and partitioned between water (pH 10) and ethyl acetate. The filtrate was basified with 1 N sodium hydroxide and extracted with ethyl acetate. The combined extracts were concentrated and purified by flash chromatography (0→20% methanol/dichloromethane) to give 1-methyl-2-(2-quinolin-3-ylpyridin-4-yl)-1,5,6,7-tetrahydro-4H-pyrrolo[3,2-c]pyridin-4-one as a white solid (80 mg, 0.225 mmol, 51% yield). ¹H NMR (400 MHz, DMSO-d₆) δ 9.67 (d, J=2.3, 1H), 9.11 (d, J=2.1, 1H), 8.73 (d, J=4.9, 1H), 8.26 (s, 1H), 8.09 (d, J=8.5, 1H), 8.06 (d, J=8.5, 1H), 7.79 (ddd, J=8.4, 6.8, 1.6, 1H), 7.65 (ddd, J=8.0, 6.9, 1.0, 1H), 7.53 (dd, J=5.1, 1.6, 1H), 7.07 (s, 1H), 6.86 (s, 1H), 3.71 (s, 3H), 3.43 (td, J=6.9, 2.6, 2H), 2.87 (t, J=6.8, 2H). HRMS calculated for C₂₂H₁₉N₄O (MH⁺) 355.1553, found 355.1580.

### EXAMPLE 395

This example illustrates the preparation of 2-{2-[3-(piperidin-1-ylmethyl)phenyl]pyridin-4-yl}-1,5,6,7-tetrahydro-4H-pyrrolo[3,2-c]pyridin-4-one trifluoroacetate. A solution of piperdine, (120 mL, 1.2 equiv.) 3-[4-(4-oxo-4,5,6,7-tetrahydro-1H-pyrrolo[3,2-c]pyridin-2-yl)pyridin-2-yl]benzaldehyde (see Example 30 above) (317 mg, 1.0 mmol) in tetrahydrofuran (7.5 mL) and acetic acid (2.5 mL) was treated with sodium triacetoxyborohydride (630 mg, 3.0 equiv) and stirred for 3 hours. The solvents were evaporated and the residue dissolved in DMSO. The product was isolated by reverse phase chromatography (81 mg, 13%) ¹H NMR (400 Hz, MeOD-d₄): δ 8.59 d, J=6.2, 1H; 8.31, d, J=1.6, 1H; 8.11, s, 1H; 8.08-8.05, m, 1H; 7.91, dd, J=6.2, 1.7, 1H; 7.76-7.74, m, 2H; 7.45, s, 1H; 4.42, s, 2H; 3.60, t, J=7.1, 2H; 3.50, bs, 2H; 3.01, t, J=7.1, 2H; 3.00, bs, 2H; 2.0-1.4, m, 6H. m/z: (M+H) 388. Calculated for C₂₄H₂₆N₄O₂(M+H): 387.2179, found 387.2140.

The following examples were prepared in a similar manner to Example 395.

| Example | | Calculated | Found |
|---|---|---|---|
| No. | Compound Name(s) | (m + H) | m + H |
| 396 | 2-{2-[3-(morpholin- | 389.1972 | 389.1989 |
| | 4-ylmethyl)phenyl]pyridin- | | |
| | 4-yl}-1,5,6,7-tetrahydro-4H- | | |
| | pyrrolo[3,2-c]pyridin-4- | | |
| | one trifluoroacetate | | |
| 397 | 2-[2-(3-{[(2-morpholin- | 432.2394 | 432.2385 |
| | 4-ylethyl)amino]methyl}phenyl) | | |
| | pyridin-4-yl]-1,5,6,7-tetrahydro- | | |
| | 4H-pyrrolo[3,2-c]pyridin-4- | | |
| | one trifluoroacetate | | |
| 398 | 2-(2-{3-[(4-methylpiperazin- | 402.2288 | 402.2288 |
| | 1-yl)methyl]phenyl}pyridin- | | |
| | 4-yl)-1,5,6,7-tetrahydro-4H- | | |
| | pyrrolo[3,2-c]pyridin- | | |
| | 4-one trifluoroacetate | | |
| 399 | 2-{2-[3-(pyrrolidin- | 373.2023 | 373.2035 |
| | 1-ylmethyl)phenyl]pyridin- | | |
| | 4-yl}-1,5,6,7-tetrahydro-4H- | | |
| | pyrrolo[3,2-c]pyridin-4- | | |
| | one trifluoroacetate | | |
| 400 | 2-[2-(3-{[(2-thien- | 429.1744 | 429.1732 |
| | 2-ylethyl)amino]methyl}phenyl) | | |
| | pyridin-4-yl]-1,5,6,7-tetrahydro- | | |
| | 4H-pyrrolo[3,2-c]pyridin-4- | | |
| | one trifluoroacetate | | |
| 401 | 2-[2-(3-{[(2- | 423.2179 | 423.2206 |
| | phenylethyl)amino]methyl} | | |
| | phenyl)pyridin-4-yl]-1,5,6, | | |
| | 7-tetrahydro-4H-pyrrolo[3, | | |
| | 2-c]pyridin-4-one trifluoroacetate | | |
| 402 | 2-{2-[3-({[2-(3- | 457.179 | 457.1766 |
| | chlorophenyl)ethyl]amino} | | |
| | methyl)phenyl]pyridin-4-yl}- | | |
| | 1,5,6,7-tetrahydro-4H-pyrrolo[3, | | |
| | 2-c]pyridin-4-one trifluoroacetate | | |
| 403 | 2-{2-[3-({[2-(3- | 441.2085 | 441.2053 |
| | fluorophenyl)ethyl]amino} | | |
| | methyl)phenyl]pyridin-4-yl}- | | |
| | 1,5,6,7-tetrahydro-4H-pyrrolo[3, | | |
| | 2-c]pyridin-4-one trifluoroacetate | | |
| 404 | 2-(2-{3-[(benzylamino)methyl] | 409.2023 | 409.2061 |
| | phenyl}pyridin-4-yl)-1,5,6, | | |
| | 7-tetrahydro-4H-pyrrolo[3, | | |
| | 2-c]pyridin-4-one trifluoroacetate | | |
| 405 | 2-[2-(3-{[(3-chlorobenzyl) | 443.1633 | 443.1623 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 406 | 2-[2-(3-{[(4-aminobenzyl) | 424.2132 | 424.2168 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 407 | 2-[2-(3-{[(2-fluorobenzyl) | 427.1929 | 427.1927 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 408 | 2-[2-(3-{[(2-chlorobenzyl) | 443.1633 | 443.1604 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 409 | 2-[2-(3-{[(4-methoxybenzyl) | 439.2129 | 439.2112 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 410 | 2-[2-(3-{[(3-fluorobenzyl) | 427.1929 | 427.1938 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 411 | 2-[2-(3-{[(3-methoxybenzyl) | 439.2129 | 439.2131 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 412 | 2-[2-(3-{[(4-fluorobenzyl) | 427.1929 | 427.1933 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |
| 413 | 2-[2-(3-{[(4-chlorobenzyl) | 443.1633 | 443.1648 |
| | amino]methyl}phenyl)pyridin- | | |
| | 4-yl]-1,5,6,7-tetrahydro-4H- | | |
| | pyrrolo[3,2-c]pyridin-4- | | |
| | one bis(trifluoroacetate) | | |

### EXAMPLE 414

This example illustrates the preparation of 2-(2-{4-[(1Z)-N-(tert-butoxy)ethanimidoyl]phenyl}pyridin-4-yl)-1,5,6,7-tetrahydro-4H-pyrrolo[3,2-c]pyridin-4-one trifluoroacetate. A mixture of 2-[2-(4-acetylphenyl)pyridin-4-yl]-1,5,6,7-tetrahydro-4H-pyrrolo[3,2-c]pyridin-4-one trifluoroacetate (Example 32) (200.0 mg, 0.60 mmol), O-(tert-butyl)hydroxylamine hydrochloride (113.0 mg, 0.90 mmol) and sodium acetate (79.0 mg, 0.96 mmol) in 1:4 mixture of water:ethanol was heated to 95° C. overnight. The crude reaction mixture was cooled to ambient temperature and filtered. The filtrate was concentrated and purified by reversed phase HPLC which gave 46.4 mg of a yellow solid. ¹H NMR (400 MHz, DMSO-d₆) δ 12.20 (br. s, 1H), 8.59 (d, J=5.6 Hz, 1H), 8.32 (s, 1H), 8.12 (d, J=8.4 Hz, 2H), 7.84 (d, J=8.4 Hz, 2H), 7.76 (m, 1H), 7.37 (s, 1H), 7.13 (s, 1H), 3.40 (m, 2H), 2.88 (t, J=6.8 Hz, 2H), 2.20 (s, 3H), 1.32 (s, 9H). m/z (M+H): 403.2.

The following examples were prepared by the same method described for Example 414.

| Example | | Calculated | Found |
|---|---|---|---|
| No. | Compound Name(s) | (m + H) | (m + H) |
| 415 | 2-(2-{4-[(1Z)-N- | 346.143 | 347.22 |
| | hydroxyethanimidoyl]phenyl}pyridin-4-yl)- | | |
| | 1,5,6,7-tetrahydro-4H-pyrrolo[3,2- | | |
| | c]pyridin-4-one | | |
| 416 | 2-(2-{4-[(1Z)-N-(2-morpholin-4-yl-2- | 473.2063 | 474.38 |
| | oxoethoxy)ethanimidoyl]phenyl}pyridin- | | |
| | 4-yl)-1,5,6,7-tetrahydro-4H-pyrrolo[3,2- | | |
| | c]pyridin-4-one trifluoroacetate | | |
| 417 | 2-(2-{4-[(1Z)-N- | 360.1586 | 361.25 |
| | methoxyethanimidoyl]phenyl}pyridin-4-yl)- | | |
| | 1,5,6,7-tetrahydro-4H-pyrrolo[3,2-c]pyridin- | | |
| | 4-one trifluoroacetate | | |
| 414 | 2-(2-{4-[(1Z)-N- | 402.2056 | 403.2 |
| | (tert-butoxy)ethanimidoyl]phenyl}pyridin-4- | | |
| | yl)-1,5,6,7-tetrahydro-4H-pyrrolo[3,2- | | |
| | c]pyridin-4-one trifluoroacetate | | |
| 418 | 2-(2-{4-[(1Z)-N- | 436.1899 | 437.28 |
| | (benzyloxy)ethanimidoyl]phenyl}pyridin-4- | | |
| | yl)-1,5,6,7-tetrahydro-4H-pyrrolo[3,2- | | |
| | c]pyridin-4-one trifluoroacetate | | |
| 419 | 2-{2-[4-((1Z)-N-{[3- | 504.1773 | 505.35 |
| | (trifluoromethyl)benzyl]oxy}ethanimidoyl)phenyl]pyridin- | | |
| | 4-yl}-1,5,6,7-tetrahydro-4H-pyrrolo[3,2- | | |
| | c]pyridin-4-one trifluoroacetate | | |
| 420 | 2-(2-{3-[(1E)-N- | 346.143 | 347.22 |
| | hydroxyethanimidoyl]phenyl}pyridin-4-yl)- | | |
| | 1,5,6,7-tetrahydro-4H-pyrrolo[3,2-c]pyridin-4-one | | |
| 421 | 2-(2-{3-[(1E)-N-(2-morpholin-4-yl- | 473.2063 | 474.38 |
| | 2-oxoethoxy)ethanimidoyl]phenyl}pyridin-4-yl)- | | |
| | 1,5,6,7-tetrahydro-4H-pyrrolo[3,2-c]pyridin-4-one | | |
| | trifluoroacetate | | |
| 422 | 2-(2-{3-[(1E)-N- | 360.1586 | 361.2 |
| | methoxyethanimidoyl]phenyl}pyridin-4-yl)- | | |
| | 1,5,6,7-tetrahydro-4H-pyrrolo[3,2-c]pyridin-4-one | | |
| | trifluoroacetate | | |
| 423 | 2-(2-{3-[(1E)-N-(tert- | 402.2056 | 403.2 |
| | butoxy)ethanimidoyl]phenyl}pyridin-4-yl)- | | |
| | 1,5,6,7-tetrahydro-4H-pyrrolo[3,2-c]pyridin-4-one | | |
| | trifluoroacetate | | |
| 424 | 2-(2-{3-[(1E)-N- | 436.1899 | 437.37 |
| | (benzyloxy)ethanimidoyl]phenyl}pyridin-4-yl)- | | |
| | 1,5,6,7-tetrahydro-4H-pyrrolo[3,2-c]pyridin-4-one | | |
| | trifluoroacetate | | |
| 425 | methyl 4-({[((1E)-1-{3-[4-(4-oxo-4,5,6,7- | 494.1954 | 495.33 |
| | tetrahydro-1H-pyrrolo[3,2-c]pyridin-2-yl)pyridin- | | |
| | 2-yl]phenyl}ethylidene)amino]oxy}methyl)benzoate | | |
| 426 | 2-{2-[3-((1E)-N-{[3- | 504.1773 | 505.31 |
| | (trifluoromethyl)benzyl]oxy}ethanimidoyl)phenyl]pyridin- | | |
| | 4-yl}-1,5,6,7-tetrahydro-4H-pyrrolo[3,2-c]pyridin- | | |
| | 4-one trifluoroacetate | | |
| 427 | methyl 4-({[((1E)-1-{4-fluoro-3-[4-(4-oxo- | 512.186 | 513.35 |
| | 4,5,6,7-tetrahydro-1H-pyrrolo[3,2-c]pyridin-2-yl)pyridin-2- | | |
| | yl]phenyl}ethylidene)amino]oxy}methyl)benzoate | | |
| | trifluoroacetate | | |
| 428 | 2-(2-{2-fluoro-5-[(1E)-N- | 364.134 | 365.24 |
| | hydroxyethanimidoyl]phenyl}pyridin-4-yl)- | | |
| | 1,5,6,7-tetrahydro-4H-pyrrolo[3,2-c]pyridin-4-one | | |
| | trifluoroacetate | | |
| 429 | 2-(2-{2-fluoro-5-[(1E)-N-(2-morpholin-4-yl- | 491.197 | 492.33 |
| | 2-oxoethoxy)ethanimidoyl]phenyl}pyridin-4-yl)- | | |
| | 1,5,6,7-tetrahydro-4H-pyrrolo[3,2-c]pyridin-4-one | | |
| | trifluoroacetate | | |

### EXAMPLE 430

This example illustrates the preparation of 2-[2-(3-butylphenyl)pyridin-4-yl]-1,5,6,7-tetrahydro-4H-pyrrolo[3,2-c]pyridin-4-one trifluoroacetate. A solution of 1-bromo-3-iodobenzene (1.2 g, 4.24 mmol) and tributylborane (1 M solution in tetrahydrofuran, 5.1 mmol, 1.2 equiv.) in tetrahydrofuran (10 mL) was treated with aqueous potassium phosphate (2M, 6.3 mL, 3 equiv.) and Pd(dppf) (69 mg, 2 mol %) and heated to 65° C. for 18 hours. After cooling, the layers were separated and the aqueous layer extracted with dichloromethane (×2). The combined organic layers were concentrated and 3-butylbromobenzene isolated by silica gel chromatography. The bromide was converted to the title compound by the method described for Example 109. ¹H NMR (300 MHz, MeOD-d₄): δ 8.51, d, J=6.6, 1H; 8.34, d, J=1.7, 1H; 8.01, dd, J=6.7, 2.0, 1H; 7.78-7.74, m, 2H; 7.61-7.52, m, 3H; 3.61, t, J=7.1, 2H; 3.03, t, J=7.0, 2H; 2.78, t, J=7.9, 2H; 1.70, pent, J=7.9, 2H; 1.44, sextet, J=7.2, 2H; 0.97, t, J=7.2, 3H. m/z 346 (M+H) Calculated for C₂₂H₂₃N₃O+H: 346.1914. Found: 346.1877.

### EXAMPLE 431

This example illustrates the preparation of 2-[2-(3-ethylphenyl)pyridin-4-yl]-1,5,6,7-tetrahydro-4H-pyrrolo[3,2-c]pyridin-4-one trifluoroacetate. The title compound was prepared from triethylborane as described for Example 430. ¹H NMR (300 MHz, MeOD-d₄): δ 8.51, d, J=6.4, 1H; 8.32, d, J=1.8, 1H; 7.98, dd, J=6.4, 2.0, 1H; 7.80, bs, 1H; 7.75, dt, J=6.8, 2.1, 1H; 7.60-7.53, m, 3H; 3.61, t, J=7.1; 2H; 3.02, t, J=7.1, 2H; 2.81, q, J=7.6, 2H; 1.33, t, J=7.7, 3H. m/z 318 (M+H) Calculated for C₂₀H₁₉N₃O+H: 318.1601. Found: 318.1573.

### EXAMPLE 432

This example illustrates the preparation of 2-(5′-methyl-2,3′-bipyridin-4-yl)-1,5,6,7-tetrahydro-4H-pyrrolo[3,2-c]pyridin-4-one trifluoroacetate was prepared by the method described for Example 430 using 3,5-dibromopyridine in lieu of 1-bromo-3-iodobenzene and trimethylboroxine in lieu of tributylborane: Calculated for C₁₈H₁₆N₄O+H: 305.1397. Found: 305.1371.

### EXAMPLE 433

This example illustrates the preparation of 2-(6′-ethyl-2,3′-bipyridin-4-yl)-1,5,6,7-tetrahydro-4H-pyrrolo[3,2-c]pyridin-4-one trifluoroacetate. The title compound was prepared by the method described for Example 430 using 2,5-dibromopyridine and triethylborane in lieu of 1-bromo-3-iodobenzene and tributylborane. Calculated for C₁₉H₁₈N₄O+H: 319.1553. Found: 319.1543.

### EXAMPLE 434

This example illustrates the preparation of 2-(6′-methyl-2,3′-bipyridin-4-yl)-1,5,6,7-tetrahydro-4H-pyrrolo[3,2-c]pyridin-4-one trifluoroacetate: The title compound was prepared by the method described for Example 430 using 2,5-dibromopyridine in lieu of 1-bromo-3-iodobenzene and trimethylboroxine in lieu of tributylborane. Calculated for C₁₈H₁₆N₄O+H: 305.1397. Found: 305.1417.

### EXAMPLE 435

This example illustrates the preparation of 2-(6′-butyl-2,3′-bipyridin-4-yl)-1,5,6,7-tetrahydro-4H-pyrrolo[3,2-c]pyridin-4-one trifluoroacetate. The title compound was prepared by the method described for Example 430 using 2,5-dibromopyridine in lieu of 1-bromo-3-iodobenzene. Calculated for C₂₁H₂₂N₄O+H: 347.1866. Found: 347.1844.

### EXAMPLE 436

This example illustrates the preparation of 2-(5′-butyl-2,3′-bipyridin-4-yl)-1,5,6,7-tetrahydro-4H-pyrrolo[3,2-c]pyridin-4-one trifluoroacetate. The title compound was prepared by the method described for Example 430 using 3,5-dibromopyridine in lieu of 1-bromo-3-iodobenzene. Calculated for C₂₁ H₂₂N₄O+H: 347.1866. Found: 347.1870.

### EXAMPLE 437

This example illustrates the preparation of 2-(5′-ethyl-2,3′-bipyridin-4-yl)-1,5,6,7-tetrahydro-4H-pyrrolo[3,2-c]pyridin-4-one trifluoroacetate. The title compound was prepared by the method described for Example 430 using 3,5-dibromopyridine in lieu of 1-bromo-3-iodobenzene and triethylborane in lieu of tributylborane. Calculated for C₁₉H₁₈N₄O+H: 319.1553. Found: 319.1583.

### EXAMPLE 438

This example illustrates the preparation of 2-{2-[3-(4-methylpentyl)phenyl]pyridin-4-yl}-1,5,6,7-tetrahydro-4H-pyrrolo[3,2-c]pyridin-4-one trifluoroacetate: To a solution of 9-BBN (0.5M solution in tetrahydrofuran, 11 mL, 5.1 mmol, 1.2 equiv) was added 4-methyl-1-pentene (644 μL, 5.1 mmol, 1.2 equiv.). The reaction mixture was stirred for 18 hours at room temperature before adding 1-bromo-3-iodobenzene, (1.2 g, 4.24 mmol) aqueous potassium phosphate (2M, 6.3 mL, 3 equiv.) and Pd(dppf)Cl₂ (69 mg, 2 mol %) and heated to 65° C. for 18 hours. After cooling, the layers were separated and the aqueous layer extracted with dichloromethane (×2). The combined organic layers were concentrated and 3-butylbromobenzene isolated by silica gel chromatography. The bromide was converted to the title compound by the method described for Example 109. ¹H NMR (300 MHz, MeOD-d₄): δ 8.51, d, J=6.4, 1H; 8.32, d, J=1.8, 1H; 7.97, dd, J=6.4, 2.0, 1H; 7.78-7.74, m, 2H; 7.60-7.51, m, 3H; 3.61, t, J=7.0; 2H; 3.02, t, J=7.0, 2H; 2.75, t, J=6.9, 2H; 1.77-1.70, m, 2H; 1.60, sextet, J=6.7, 1H; 1.34-1.24, m, 2H; 0.90, d, J=6.7, 6H. m/z 374 (M+H) Calculated for C₂₄H₂₇N₃O+H: 374.2227. Found: 374.2237.

### EXAMPLE 439

This example illustrates the preparation of 2-[2-(3-isobutylphenyl)pyridin-4-yl]-1,5,6,7-tetrahydro-4H-pyrrolo[3,2-c]pyridin-4-one trifluoroacetate was prepared by the method described for Example 438 using 2-methyl propene in lieu of 4-methyl-1-pentene. Calculated for C₂₂H₂₃N₃O+H: 346.1914. Found: 346.1915.

### EXAMPLE 440

This example illustrates the preparation of 2-(5′-isobutyl-2,3′-bipyridin-4-yl)-1,5,6,7-tetrahydro-4H-pyrrolo[3,2-c]pyridin-4-one bis(trifluoroacetate) was prepared by the method described for Example 438 using 2-methyl propene in lieu of 4-methyl-1-pentene and 3,5-dibromopyridine in lieu of 1-bromo-3-iodobenzene. Calculated for C₂₁H₂₂N₄O+H: 347.1866. Found: 347.1831.

### EXAMPLE 441

This example illustrates the preparation of 2-[5′-(2-cyclopentylethyl)-2,3′-bipyridin-4-yl]-1,5,6,7-tetrahydro-4H-pyrrolo[3,2-c]pyridin-4-one trifluoroacetate: The title compound was prepared by the method described for Example 438 using cyclopentylethylene in lieu of 4-methyl-1-pentene and 3,5-dibromopyridine in lieu of 1-bromo-3-iodobenzene. Calculated for C₂₄H₂₆N₄O+H: 387.2179. Found: 387.2221.

### EXAMPLE 442

This example illustrates the preparation of 2-[5′-(4-methylpentyl)-2,3′-bipyridin-4-yl]-1,5,6,7-tetrahydro-4H-pyrrolo[3,2-c]pyridin-4-one trifluoroacetate: The title compound was prepared by the method described for Example 438 using 3,5-dibromopyridine in lieu of 1-bromo-3-iodobenzene. Calculated for C₂₃H₂₆N₄O+H: 375.2179. Found: 375.2176.

The following examples were prepared by the method described for example 438 using 3,5-dibromopyridine in lieu of 1-bromo-3-iodobenzene and the appropriate alkene.

| Example | | Calculated | Found |
|---|---|---|---|
| No. | Compound Name(s) | (M + H) | (M + H) |
| 443 | tert-butyl 3-[4-(4-oxo-4,5,6,7-tetrahydro-1H- | 419.2078 | 419.2094 |
| | pyrrolo[3,2-c]pyridin-2-yl)-2,3′- | | |
| | bipyridin-6′-yl]propanoate trifluoroacetate | | |
| 444 | ethyl 5-[4-(4-oxo-4,5,6,7-tetrahydro-1H- | 419.2078 | 419.2062 |
| | pyrrolo[3,2-c]pyridin-2-yl)-2,3′- | | |
| | bipyridin-6′-yl]pentanoate trifluoroacetate | | |
| 445 | 2-{6′-[2-(4-fluorophenyl)ethyl]-2,3′- | 413.1772 | 413.1742 |
| | bipyridin-4-yl}-1,5,6,7-tetrahydro-4H- | | |
| | pyrrolo[3,2-c]pyridin-4-one trifluoroacetate | | |
| 446 | 2-[6′-(2-cyclopentylethyl)-2,3′-bipyridin- | 387.2179 | 387.2186 |
| | 4-yl]-1,5,6,7-tetrahydro-4H-pyrrolo[3,2- | | |
| | c]pyridin-4-one trifluoroacetate | | |
| 447 | 2-[6′-(3-methoxypropyl)-2,3′-bipyridin-4- | 363.1816 | 363.1829 |
| | yl]-1,5,6,7-tetrahydro-4H-pyrrolo[3,2- | | |
| | c]pyridin-4-one trifluoroacetate | | |
| 448 | 2-[6′-(3-aminopropyl)-2,3′-bipyridin-4- | 348.1819 | 348.1822 |
| | yl]-1,5,6,7-tetrahydro-4H-pyrrolo[3,2- | | |
| | c]pyridin-4-one trifluoroacetate | | |
| 449 | 2-{6′-[3-(allylamino)propyl]-2,3′- | 388.2132 | 388.2136 |
| | bipyridin-4-yl}-1,5,6,7-tetrahydro-4H- | | |
| | pyrrolo[3,2-c]pyridin-4-one trifluoroacetate | | |
| 450 | 2-{4-[4-(4-oxo-4,5,6,7-tetrahydro-1H- | 492.2030 | 492.2014 |
| | pyrrolo[3,2-c]pyridin-2-yl)-2,3′-bipyridin- | | |
| | 6′-yl]butyl}-1H-isoindole-1,3(2H)-dione | | |
| | trifluoroacetate | | |
| 451 | 2-[6′-(5-hydroxypentyl)-2,3′-bipyridin-4- | 377.1972 | 377.1985 |
| | yl]-1,5,6,7-tetrahydro-4H-pyrrolo[3,2- | | |
| | c]pyridin-4-one trifluoroacetate | | |
| 452 | 4-[4-(4-oxo-4,5,6,7-tetrahydro-1H-pyrrolo[3,2- | 358.1662 | 358.1639 |
| | c]pyridin-2-yl)-2,3′-bipyridin-6′- | | |
| | yl]butanenitrile trifluoroacetate | | |

### EXAMPLE 453

This example illustrates the preparation of 3-[4-(4-oxo-4,5,6,7-tetrahydro-1H-pyrrolo[3,2-c]pyridin-2-yl)-2,3′-bipyridin-6′-yl]propanoic acid trifluoroacetate. A solution of tert-butyl 3-[4-(4-oxo-4,5,6,7-tetrahydro-1H-pyrrolo[3,2-c]pyridin-2-yl)-2,3′-bipyridin-6′-yl]propanoate trifluoroacetate (Example 443) (300 mg, 0.46 mmol) in methanol (5 mL) was treated with 5% aqueous sodium hydroxide (5 mL) and stirred for 18 hr. The solution was neutralized with 5% aqueous hydrochloric acid (5 mL) and the methanol removed by evaporation. The product was isolated by reverse phase chromatography. ¹H NMR (300 MHz, DMSO-d₆): δ 12.48, s, 1H; 9.25, s, 1H; 8.67, s, 1H; 8.66, s, 1H; 8.45, s, 1H; 7.85, d, J=5.8, 1H; 7.79, d, J=8.3, 1H; 7.47, s, 1H; 7.17, s, 1H; 3.42, t, J=6.8, 2H; 3.15, t, J=7.1; 2H; 2.89, t, J=6.6, 2H; 2.79, t, J=7.1, 2H. m/z 363 (M+H) Calculated for C₂₀H₁₈N₄O₃+H: 363.1452. Found: 363.1454.

### EXAMPLE 454

This example illustrates the preparation of 5-[4-(4-oxo-4,5,6,7-tetrahydro-1H-pyrrolo[3,2-c]pyridin-2-yl)-2,3′-bipyridin-6′-yl]pentanoic acid: The title compound was prepared by the same method described for Example 453 from ethyl 5-[4-(4-oxo-4,5,6,7-tetrahydro-1H-pyrrolo[3,2-c]pyridin-2-yl)-2,3′-bipyridin-6′-yl]pentanoate trifluoroacetate (Example 453). Calculated for C₂₂H₂₂N₄O₃+H: 391.1765. Found: 391.1779.

### EXAMPLE 455

This example illustrates the preparation of 2-{6′-[3-(cyclobutylamino)propyl]-2,3′-bipyridin-4-yl}-1,5,6,7-tetrahydro-4H-pyrrolo[3,2-c]pyridin-4-one trifluoroacetate. A solution of 2-[6′-(3-aminopropyl)-2,3′-bipyridin-4-yl]-1,5,6,7-tetrahydro-4H-pyrrolo[3,2-c]pyridin-4-one trifluoroacetate (Example 448) (0.29 mmol) in acetic acid (0.75 mL) and tetrahydrofuran (0.75 mL) was treated with cyclobutanone (32 μL, 1.5 equiv.) and sodium triacetoxyborohydride (184 mg, 3.0 equiv.) and stirred at 35° C. for 18 hours. The mixture was diluted with methanol (10 mL) and the product isolated by reverse phase chromatography. ¹H NMR (300 MHz, DMSO-d₆): δ 12.14, s, 1H; 9.24, s, 1H; 8.71, bs, 1H; 8.62, d, J=5.6, 1H; 8.51, d, J=6.4, 1H; 8.34, s, 1d, 7.71, d, J=4.3, 1H; 7.53, d, J=8.4, 1H; 7.34, s, 1H; 7.11, bs, 1H; 3.7-3.67, m, 1H; 3.41, t, J=6.5, 2H; 2.94-2.58, m, 6H; 2.15-1.99, m, 6H; 1.78, t, J=8.5, 2H. m/z 402 (M+H) Calculated for C₂₄H₂₇N₅O+H: 402.2288. Found: 402.2272.

The following examples were prepared by the method described for Example 455.

| Example | | Calculated | Found |
|---|---|---|---|
| No. | Compound Name(s) | (m + H) | (m + H) |
| 456 | N-{3-[4-(4-oxo-4,5,6,7-tetrahydro-1H- | 390.1925 | 390.1928 |
| | pyrrolo[3,2-c]pyridin-2-yl)-2,3′- | | |
| | bipyridin-6′-yl]propyl}acetamide | | |
| | trifluoroacetate | | |
| 457 | 2-(6′-{3-[(cyclohexylmethyl)amino]propyl}- | 444.2758 | 444.2769 |
| | 2,3′-bipyridin-4-yl)-1,5,6,7-tetrahydro-4H- | | |
| | pyrrolo[3,2-c]pyridin-4-one trifluoroacetate | | |
| 458 | 2-(6′-{3-[(quinolin-4-ylmethyl)amino]propyl}- | 489.2397 | 489.2357 |
| | 2,3′-bipyridin-4-yl)-1,5,6,7-tetrahydro-4H- | | |
| | pyrrolo[3,2-c]pyridin-4-one trifluoroacetate | | |
| 459 | 2-(6′-{3-[(3-methylbutyl)amino]propyl}- | 418.2601 | 418.2583 |
| | 2,3′-bipyridin-4-yl)-1,5,6,7-tetrahydro-4H- | | |
| | pyrrolo[3,2-c]pyridin-4-one trifluoroacetate | | |
| 460 | 2-(6′-{3-[bis(3-methylbutyl)amino]propyl}- | 488.3384 | 488.3371 |
| | 2,3′-bipyridin-4-yl)-1,5,6,7-tetrahydro-4H- | | |
| | pyrrolo[3,2-c]pyridin-4-one trifluoroacetate | | |
| 461 | 2-(6′-{3-[(3-phenylpropyl)amino]propyl}- | 466.2601 | 466.258 |
| | 2,3′-bipyridin-4-yl)-1,5,6,7-tetrahydro-4H- | | |
| | pyrrolo[3,2-c]pyridin-4-one trifluoroacetate | | |
| 462 | 2-(6′-{3-[bis(3-phenylpropyl)amino]propyl}- | 584.3384 | 584.3444 |
| | 2,3′-bipyridin-4-yl)-1,5,6,7-tetrahydro-4H- | | |
| | pyrrolo[3,2-c]pyridin-4-one trifluoroacetate | | |

### EXAMPLE 463

This example illustrates the preparation of 2-quinolin-3-yl-8,9,10,11-tetrahydro-7H-pyrido[3′,4′:4,5]pyrrolo[2,3-f]isoquinolin-7-one.

Step 1. (Preparation of 3-(5-Nitroisoquinolin-3-yl)quinoline).

A mixture of tetrakis(triphenylphospine)palladium(0) (1.4 g, 1.2 mmol), 3-chloro-5-nitroisoquinoline (Serban, A. U.S. Pat. No. 3,930,837 1/1976) (5.00 g, 24.0 mmol), quinoline-3-boronic acid (4.60 g, 26.4 mmol), 2 M aqueous sodium carbonate (36 mL), toluene (100 mL), and ethanol (100 mL) was refluxed under nitrogen for 90 minutes. The reaction mixture was diluted with water (500 mL) and ethyl acetate (400 mL). A precipated formed. The precipated was filtered and washed with ethyl acetate to give the title compound (2.50 g) as yellow needles. The filtrate was extracted with ethyl acetate, and the organic layers were combined, washed with brine, dried over sodium sulfate, and concentrated to give a solid. The solid was recrystallized from acetonitrile/ethanol to give the title compound (2.82 g) as yellow needles. Total yield of the title compound was (5.32 g, 17.7 mmol, 74%): ¹H NMR (400 MHz, DMSO-d₆) δ 9.73 (d, J=0.6 Hz, 1H), 9.71 (d, J=2.3 Hz, 1H), 9.17 (d, J=2.2 Hz, 1H), 9.02 (s, 1H), 8.70 (dd, J=7.7, 1.1 Hz, 1H), 8.67 (d, J=8.0 Hz, 1H), 8.21 (d, J=7.5 Hz, 1H), 8.10 (d, J=8.3 Hz, 1H), 7.92 (t, J=7.9 Hz, 1H), 7.84 (td, J=7.8, 1.4 Hz, 1H), 7.69 (td, J=8.0, 1.0 Hz, 1H). m/z 302 (M+H).

Step 2. (Preparation of 3-Quinolin-3-yl-isoquinolin-5-amine).

A mixture of 3-(5-nitroisoquinolin-3-yl)quinoline), prepared as described above, (2.34 g, 7.77 mmol), 10% Pd/C (degussa, 900 mg), concentrated hydrochloric acid (4 mL), and methanol (100 mL) was placed under 30 psi hydrogen gas on a Parr Shaker apparatus for 2.5 hours. The mixture was filtered through celite. The celite cake was washed with methanol repeatedly (800 mL total). The filtrated was concentrated to give a red solid. The red solid was boiled in methanol (200 mL). After cooling to room temperature, the resultant solid was filtered and washed with cold methanol and ether to give the title compound as an orange solid (1.33 g, 4.90 mmol, 63% yield, >90% pure) which was used without further purification: ¹H NMR (400 MHz, DMSO-d₆) δ 10.01 (d, J=2.1 Hz, 1H), 9.73 (s, 1H), 9.44 (s, 1H), 9.14 (s, 1H), 8.36 (t, J=8.6 Hz, 2H), 8.06 (td, J=7.8, 1.3 Hz, 1H), 7.91 (t, J=7.6 Hz, 1H), 7.58-7.52 (m, 2H), 7.16 (dd, J=6.3, 2.3 Hz, 1H). m/z 272 (M+H).

Step 3. (Preparation of 2-quinolin-3-yl-8,9,10,11-tetrahydro-7H-pyrido[3′,4′:4,5]pyrrolo[2,3-f]isoquinolin-7-one).

To a suspension of 3-quinolin-3-yl-isoquinolin-5-amine (385 mg, 1.42 mmol) in concentrated hydrochloric acid (4 mL) in an ice-salt bath was added a solution of sodium nitrite (100 mg, 1.42 mmol) in water (1 mL) dropwise. After 10 minutes, the dark solution was added to a solution of tin(II) chloride dihydrate (960 mg, 4.26 mmol) in concentrated hydrochloric acid (4 mL) in an ice-salt bath. The reaction mixture was allowed to warm to room temperature over 15 minutes and then was poured into ice-water (100 mL). The dark solution was treated with concentrated ammonium hydroxide until basic (pH=9). The resultant hydrazine precipitate was filtered and washed with water. The hydrazine precipitate and 2,4-dioxopiperidine (161 mg, 1.42 mmol) were refluxed in ethanol (15 mL) for 4 hours. The reaction mixture was cooled to room temperature and filtered. The precipate was washed with ethanol to give the hydrazone as a yellow solid. The hydrazone was suspended in glacial acetic acid (15 mL) and was treated with concentrated sulfuric acid (2 mL). The suspension was refluxed for 45 minutes, cooled, diluted with ethyl acetate (10 mL), and filtered. The solids were extracted with N,N-dimethylformamide and purified by reverse-phase high pressure chromatography (5 to 70% acetonitrile/water/0.05% trifluoroacetic acid). The pure fractions were lyophilized to give the title compound as a yellow solid (29 mg, 0.0489 mmol, 3.4% yield): ¹H NMR (400 MHz, DMSO-d₆) δ 12.93 (s, 1H), 9.72 (d, J=2.0 Hz, 1H), 9.52 (s, 1H), 9.15 (d, J=1.9 Hz, 1H), 9.11 (s, 1H), 8.26 (d, J=8.6 Hz, 1H), 8.20 (d, J=8.2 Hz, 1H), 8.15 (d, J=8.5 Hz, 1H), 7.88-7.85 (m, 2H), 7.74 (t, J=7.0 Hz, 1H), 7.30 (s, 1H), 3.56 (t, J=6.7 Hz, 1H), 3.17 (t, J=6.9 Hz, 1H). HRMS calculated for C₂₃H₁₇N₄O (MH⁺) 365.1397, found 365.1421.

### EXAMPLE 464

This example illustrates the preparation of (2E)-3-{3-[4-(4-oxo-4,5,6,7-tetrahydro-1H-pyrrolo[3,2-c]pyridin-2-yl)pyridin-2-yl]phenyl}acrylonitrile. To a slurry of 3-[4-(4-oxo-4,5,6,7-tetrahydro-1H-pyrrolo[3,2-c]pyridin-2-yl)pyridin-2-yl]benzaldehyde (Example 30) (303.4 mg, 0.95 mmol) and potassium carbonate (396.0 mg, 2.9 mmol) in 3.2 ml wthanol was added siethyl cyanomethylphosphonate (189 μl, 1.15 mmol). The reaction mixture was heated to 80° C. for 5 hours. The mixture was cooled to ambient temperature and solids filtered. The solids were washed with water and methanol revealing 151.7 mg tan solids. ¹HNMR (400 MHz, DMSO-d₆) δ 11.94 (s, 1H), 8.53 (d, J=2.4 Hz, 1H), 8.19 (d, J=6.4 Hz, 2H), 7.70 (m, 2H), 7.56 (m, 2H), 7.17 (d, J=2.4 Hz, 1H), 7.02 (s, 1H), 6.56 (d, J=16.8 Hz, 1H), 4.52 (t, J=5.6 Hz, 2H), 2.83 (t, J=6.8 Hz, 2H). m/z (M+H) 341.23.

### EXAMPLE 465

This example illustrates the preparation of 2-(2,5-dichloropyridin-4-yl)-1,5,6,7-tetrahydro-4H-pyrrolo[3,2-c]pyridin-4-one. The following procedure illustrates the general method for preparing the title compound.

Step 1: Preparation of 1-[2,5-dichloropyridin-4-yl]ethanone.

A 2.5 M BuLi solution in hexanes (10 mL, 25 mmol) was added to a solution of diisopropylamine in THF (10 mL) at −75^{□}° C. and the reaction mixture was stirred at that temperature for 45 min. 2,5-Dichloropyridine (2.0 g, 13.5 mmol) dissolved in THF (5 mL) was added dropwise to above solution and stirred at that temperature for 1 h under N₂ atm. A solution of N-methoxy-N-methylacetamide (1.50 g, 14.54 mmol) in THF (5 mL) was added at −75° C. and the mixture was stirred for 30 min. The reaction mixture was quenched with a cold aqueous NH₄Cl solution and was extracted with ether (3×50 mL). The combined organic layers were washed with water, saturated brine solution, dried with anhydrous MgSO₄ and evaporated in vacuo. The crude product was purified by flash chromatography on SiO₂ gel using ethylacetate (0-5%) in hexanes to elute the 1-[2,5-dichloropyridin-4-yl]ethanone as a cream crystalline solid (1.228 g, 48%). ¹H NMR (300 MHz, CDCl₃): δ 2.65 (s, 3H), 7.40 (s, 1H), 8.45 (s, 1H). ¹³C NMR (75 MHz, CDCl₃): δ 30.26, 122.81, 126.49, 147.63, 150.40, 197.13. GC-MS, m/z 189 (³⁵CIM⁺), 191 (³⁷CIM⁺). High resolution MS calculated for C₇H₆Cl₂NO (M+H)=189.9821. Found 189.9849.

Step 2: Preparation of 2-bromo-1-[2,5-dichloropyridin-4-yl]ethanone.

2-Bromo-1-[2,5-dichloropyridin-4-yl]ethanone was prepared by the general procedure used for bromination of the 4-acetyl-2-chloropyridine. ¹H NMR (300 MHz, DMSO-d₆) δ 4.91 (s, 2H), 8.03 (s, 1H), 8.67 (s, 1H). Positive electrospray LC-MS, m/z 268, 270, 272 (M+H⁺).

Step 3: Preparation of 2-(2,5-dichloropyridin-4-yl)-1,5,6,7-tetrahydro-4H-pyrrolo[3,2-c]pyridin-4-one.

The compound was prepared using the general procedure used for the preparation of 2-(2-chloropyridin-4-yl)-1,5,6,7-tetrahydro-4H-pyrrolo[3,2-c]pyridin-4-one and was isolated as a cream solid. ¹H NMR (400 MHz, DMSO-d₆): δ 2.86 (t, 2H), 3.41 (dt, 2H), 7.17 (brs, 1H), 7.25 (d, 1H), 7.78 (s, 1H), 8.45 (s, 1H), 12.04 (brs, 1H). ¹³C NMR (100 MHz, DMSO-d₆): δ 21.71, 39.94, 111.75, 115.94, 120.24, 123.96, 125.21, 139.37, 140.09, 148.99, 150.31, 164.36. Positive electrospray LC-MS, m/z 282 (³⁵CIM+H⁺), 284 (³⁷CIM+H⁺). High resolution MS calculated for C₁₂H10Cl₂N₃O (M+H)=282.0195. Found 282.0196.

2-(2,5-dichloropyridin-4-yl)-1,5,6,7-tetrahydro-4H-pyrrolo[3,2-c]pyridin-4-one trifluoroacetate was prepared by dissolving the 2-(2,5-dichloropyridin-4-yl)-1,5,6,7-tetrahydro-4H-pyrrolo[3,2-c]pyridin-4-one in a mixture of DMF/water (1:1) containing a few drops of CF₃COOH. The crude residue was purified by reverse-phase C18 chromatography with a water/acetonitrile gradient containing 0.1% TFA. Positive electrospray LC-MS, m/z 282 (³⁵CIM+H⁺), 284 (³⁷CIM+H⁺).

Step 4: Preparation of 2-(2-aryl-5-chloropyridin-4-yl)-1,5,6,7-tetrahydro-4H-pyrrolo[3,2-c]pyridin-4-ones

The following compounds were prepared using the general procedure of cross-coupling commercially available boronic acids with 2-(2,5-dichloropyridin-4-yl)-1,5,6,7-tetrahydro-4H-pyrrolo[3,2-c]pyridin-4-one. The purified products were characterized by analytical reverse phase HPLC, LC-MS, ¹H NMR, ¹³C NMR, ¹⁹F NMR and HR-MS.

| Example | | Calculated | Found |
|---|---|---|---|
| No. | Compound Name(s) | (m + H) | (m + H) |
| 465 | 2-(2,5-dichloropyridin-4-yl)-1,5,6,7-tetrahydro- | 282.0195 | 282.0196 |
| | 4H-pyrrolo[3,2-c]pyridin-4-one | | |
| 465 | 2-(2,5-dichloropyridin-4-yl)-1,5,6,7-tetrahydro- | 282.0195 | 282 (ES) |
| | 4H-pyrrolo[3,2-c]pyridin-4-one | | |
| | trifluoroacetate | | |
| 466 | 2-{5-chloro-2-[(E)-2-phenylvinyl]pyridin- | 350.1055 | 350.1019 |
| | 4-yl}-1,5,6,7-tetrahydro-4H-pyrrolo[3,2- | | |
| | c]pyridin-4-one trifluoroacetate | | |
| 467 | 2-[5-chloro-2-(2-fluorophenyl)pyridin-4-yl]- | 342.0804 | 342.0813 |
| | 1,5,6,7-tetrahydro-4H-pyrrolo[3,2-c]pyridin- | | |
| | 4-one trifluoroacetate | | |
| 468 | 2-(5-chloro-2-quinolin-3-ylpyridin-4-yl)-1,5,6,7- | 375.1007 | 375.1033 |
| | tetrahydro-4H-pyrrolo[3,2-c]pyridin-4-one | | |
| | trifluoroacetate | | |
| 469 | 2-[2-(3-aminophenyl)-5-chloropyridin-4-yl]- | 339.1007 | 339.1002 |
| | 1,5,6,7-tetrahydro-4H-pyrrolo[3,2-c]pyridin- | | |
| | 4-one trifluoroacetate | | |

### EXAMPLE 470

This example illustrates the preparation of 2-(2-chloro-5-fluoropyridin-4-yl)-1,5,6,7-tetrahydro-4H-pyrrolo[3,2-c]pyridin-4-one: The following procedure illustrates the general method for preparing the title compound.

Step 1: Preparation of 1-[2-chloro-5-fluoropyridin-4-yl]ethanone

A solution of 2.5 M BuLi in hexanes (10 mL, 25 mmol) was added to a solution of N,N,N′,N″,N″-pentamethyldiethylenetriamine in THF (10 mL) at −75° C. and the reaction mixture was stirred at that temperature for 40 min. 2-Chloro-5-fluoropyridine (3.25 g, 24.7 mmol) was added dropwise to above solution and stirred for 1 h. N-Methoxy-N-methylacetamide (2.65 mL, 24.9 mmol) was added at −75° C. and the mixture was stirred for 45 min. The reaction mixture was quenched with a cold aqueous NH₄Cl solution and was extracted with CH₂Cl₂ (3×50 mL). The combined organic layers were washed with water, saturated brine solution, dried with anhydrous MgSO₄ and evaporated in vacuo. The crude product was purified by flash chromatography on SiO₂ gel using ethylacetate (0-5%) in hexanes to elute the 1-[2-chloro-5-fluoropyridin-4-yl]ethanone as a pale yellow liquid (1.474 g, 35%). ¹H NMR (400 MHz, CDCl₃): δ 2.68 (d, J=4.0 Hz, 3H), 7.68 (d, J=5.1 Hz, 1H), 8.42 (d, J=1.88 Hz, 1H). ¹³C NMR (100 MHz, CDCl₃): δ 31.15 (d, J=6.3 Hz), 123.58, 133.84 (d, J=13.4 Hz), 139.90 (d, J=29.2 Hz), 147.26 (d, J=3.25 Hz), 156.30 (d, J=262.3 Hz), 193.13 (d, J=3.25 Hz). ¹⁹F NMR (376 MHz, CDCl₃): δ −129.72 (q). GC-MS, m/z 173 (³⁵CIM+), 175 (³⁷CIM⁺). High resolution MS calculated for C₇H₆ClFNO (M+H)=174.0116. Found 174.0190.

Step 2: Preparation of 2-bromo-1-[2-chloro-5-fluoropyridin-4-yl]ethanone

2-Bromo-1-[2-chloro-5-fluoropyridin-4-yl]ethanone was prepared by the general procedure used for bromination of the 4-acetyl-2-chloropyridine. ¹H NMR (300 MHz, DMSO-d₆) δ 4.92 (d, J=1.5 Hz, 2H), 7.95 (d, J=5.14 Hz, 1H), 8.69 (d, J=2.5 Hz, 1H), 10.43 (brs, 1H). ¹⁹F NMR (282 MHz, DMSO-d₆): δ −129.91. Positive electrospray LC-MS, m/z 252, 254, 256 (M+H⁺).

Step 3: Preparation of 2-(2-chloro-5-fluoropyridin-4-yl)-1,5,6,7-tetrahydro-4H-pyrrolo[3,2-c]pyridin-4-one.

The compound was prepared using the general procedure used for the preparation of 2-(2-chloropyridin-4-yl)-1,5,6,7-tetrahydro-4H-pyrrolo[3,2-c]pyridin-4-one. ¹H NMR (300, MHz, DMSO-d₆): δ 2.86 (t, J=6.85 Hz, 2H), 3.41 (dt, J=6.85 and 2.50 Hz, 2H), 6.99 (t, J=2.8 Hz, 1H), 7.17 (brs, 1H), 7.86 (d, J=5.84 Hz, 1H), 8.39 (d, J=3.2 Hz, 1H), 12.03 (brs, 1H). ¹³C NMR (75 MHz, DMSO-d₆): δ 21.73, 39.93, 111.48 (d, J=11.15 Hz), 116.25 (d, J=1.85 Hz), 118.49 (d, J=1.85 Hz), 121.41 (d, J=3.15 Hz), 129.91 (d, J=11.44 Hz), 138.17 (d, J=27.87 Hz), 140.29, 145.82 (d, J=2.57 Hz), 153.96 (d, J=254.86 Hz), 164.31. ¹⁹F NMR (282 MHz, DMSO-d₆): δ −134.00 (q). Positive electrospray LC-MS, m/z 266 (³⁵CIM+H⁺), 268 (³⁷CIM+H⁺). High resolution MS calculated for C₁₂H₁₀ClFN₃O (M+H)=266.0491. Found 266.0528.

2-(2-chloro-5-fluoropyridin-4-yl)-1,5,6,7-tetrahydro-4H-pyrrolo[3,2-c]pyridin-4-one trifluoroacetate was prepared by dissolving the 2-(2-chloro-5-fluoropyridin-4-yl)-1,5,6,7-tetrahydro-4H-pyrrolo[3,2-c]pyridin-4-one in a mixture of DMF/water (1:1) containing a few drops of CF₃COOH. The crude residue was purified by reverse-phase C18 chromatography with a water/acetonitrile gradient containing 0.1% TFA to afford the desired product. ¹H NMR (300, MHz, DMSO-d₆): δ 2.86 (t, J=6.85 Hz, 2H), 3.41 (dt, J=6.94 and 2.50 Hz, 2H), 6.99 (t, J=2.97 Hz, 1H), 7.17 (brs, 1H), 7.87 (d, J=5.94 Hz, 1H), 8.40 (d, J=3.2 Hz, 1H), 12.04 (brs, 1H). ¹⁹F NMR (282 MHz, DMSO-d₆): δ −134.00 (q), −74.22 (TFA). Positive electrospray LC-MS, m/z 266 (³⁵CIM+H⁺), 268 (³⁷CIM+H⁺).

Step 4: Preparation of 2-(2-aryl-5-fluoropyridin-4-yl)-1,5,6,7-tetrahydro-4H-pyrrolo[3,2-c]pyridin-4-ones

The following compounds were prepared using the general procedure of cross-coupling commercially available boronic acids with 2-(2,5-dichloropyridin-4-yl)-1,5,6,7-tetrahydro-4H-pyrrolo[3,2-c]pyridin-4-one.

The purified products were characterized by analytical reverse phase HPCL, LC-MS, ¹H NMR, ¹³C NMR, ¹⁹F NMR and HR-MS.

| Example | | Calculated | Found |
|---|---|---|---|
| No. | Compound Name(s) | (m + H) | (m + H) |
| 470 | 2-(2-chloro-5-fluoropyridin-4-yl)-1,5,6,7- | 266.0491 | 266.0528 |
| | tetrahydro-4H-pyrrolo[3,2-c]pyridin-4-one | | |
| 470 | 2-(2-chloro-5-fluoropyridin-4-yl)-1,5,6,7- | 266.0491 | 266 (ES) |
| | tetrahydro-4H-pyrrolo[3,2-c]pyridin-4-one | | |
| | trifluoroacetate | | |
| 471 | 2-[5-fluoro-2-(2-fluorophenyl)pyridin-4-yl]- | 326.1009 | 326.1129 |
| | 1,5,6,7-tetrahydro-4H-pyrrolo[3,2-c]pyridin- | | |
| | 4-one trifluoroacetate | | |
| 472 | 2-{5-fluoro-2-[(E)-2-phenylvinyl]pyridin- | 334.135 | 334.1349 |
| | 4-yl}-1,5,6,7-tetrahydro-4H-pyrrolo[3,2- | | |
| | c]pyridin-4-one trifluoroacetate | | |
| 473 | 2-(5-fluoro-2-phenylpyridin-4-yl)-1,5,6,7- | 308.1194 | 308.1181 |
| | tetrahydro-4H-pyrrolo[3,2-c]pyridin-4-one | | |
| | trifluoroacetate | | |
| 474 | 2-[5-fluoro-2-(4-methoxyphenyl)pyridin-4-yl]- | 338.1299 | 338.1288 |
| | 1,5,6,7-tetrahydro-4H-pyrrolo[3,2-c]pyridin- | | |
| | 4-one trifluoroacetate | | |
| 475 | 2-[5-fluoro-2-(4-hydroxyphenyl)pyridin-4-yl]- | 324.1143 | 324.1128 |
| | 1,5,6,7-tetrahydro-4H-pyrrolo[3,2-c]pyridin- | | |
| | 4-one trifluoroacetate | | |
| 476 | 4-[5-fluoro-4-(4-oxo-4,5,6,7-tetrahydro-1H- | 352.1092 | 352.1118 |
| | pyrrolo[3,2-c]pyridin-2-yl)pyridin-2- | | |
| | yl]benzoic acid trifluoroacetate | | |
| 477 | 2-[2-(3-acetylphenyl)-5-fluoropyridin-4-yl]- | 350.1299 | 350.1279 |
| | 1,5,6,7-tetrahydro-4H-pyrrolo[3,2-c]pyridin- | | |
| | 4-one trifluoroacetate | | |
| 478 | 2-[5-fluoro-2-(4-phenoxyphenyl)pyridin-4-yl]- | 400.1456 | 400.1466 |
| | 1,5,6,7-tetrahydro-4H-pyrrolo[3,2-c]pyridin- | | |
| | 4-one trifluoroacetate | | |
| 479 | 2-[2-(3-aminophenyl)-5-fluoropyridin-4-yl]- | 323.1303 | 323.1337 |
| | 1,5,6,7-tetrahydro-4H-pyrrolo[3,2-c]pyridin- | | |
| | 4-one trifluoroacetate | | |
| 480 | 2-[2-(3-chloro-4-fluorophenyl)-5-fluoropyridin- | 360.071 | 360.074 |
| | 4-yl]-1,5,6,7-tetrahydro-4H-pyrrolo[3,2- | | |
| | c]pyridin-4-one trifluoroacetate | | |
| 481 | 2-(5-fluoro-2-thien-2-ylpyridin-4-yl)-1,5,6,7- | 315.0758 | 314.0776 |
| | tetrahydro-4H-pyrrolo[3,2-c]pyridin-4-one | | |
| | trifluoroacetate | | |
| 482 | 2-(5-fluoro-2-quinolin-3-ylpyridin-4-yl)-1,5,6,7- | 359.1303 | 359.1262 |
| | tetrahydro-4H-pyrrolo[3,2-c]pyridin-4-one | | |
| | trifluoroacetate | | |
| 483 | 2-[5-fluoro-2-(3-fluorophenyl)pyridin-4-yl]- | 326.1099 | 326.1053 |
| | 1,5,6,7-tetrahydro-4H-pyrrolo[3,2-c]pyridin- | | |
| | 4-one trifluoroacetate | | |
| 484 | 2-fluoro-4-[5-fluoro-4-(4-oxo-4,5,6,7-tetrahydro- | 370.0998 | 370.1006 |
| | 1H-pyrrolo[3,2-c]pyridin-2-yl)pyridin-2- | | |
| | yl]benzoic acid | | |

### EXAMPLE 485

This example illustrates the preparation of 2-fluoro-N-(3-fluorobenzyl)-4-[5-fluoro-4-(4-oxo-4,5,6,7-tetrahydro-1H-pyrrolo[3,2-c]pyridin-2-yl)pyridin-2-yl]benzamide.

2-Fluoro-4-[5-fluoro-4-(4-oxo-4,5,6,7-tetrahydro-1H-pyrrolo[3,2-c]pyridin-2-yl)pyridin-2-yl]benzoic acid (Example 484) (0.0407 g, 0.11 mmol) and HBTU (0.0912 g, 0.24 mmol) were placed in an oven dried vial under N₂ atm and dissolved in dry DMSO (1 mL). 0.1 mL (0.574 mmol) of the DIEA was added and the reaction mixture was stirred at room temperature (rt) for 60 min. 3-Fluorobenzylamine (0.03 mL, 0.263 mmol) was added and the reaction mixture was stirred at rt for 1 h. The reaction was complete by analytical HPLC and LC-MS. The crude residue was purified by reverse-phase C¹⁸ chromatography with a water/acetonitrile gradient and lyophilized to afford the title compound as a colorless solid (43 mg). ¹H NMR (400 MHz, CD₃OD): δ 2.99 (t, J=7 0 Hz, 2H), 3.59 (t, J=7.0 Hz, 2H), 4.61 (s, 2H), 6.99 (m, 1H), 7.12 (m, 1H), 7.20 (m, 1H), 7.22 (d, J=2.6 Hz 1H), 7.35 (ddd, 1H), 7.85 (t, J=7 8 Hz, 1H), 7.90-7.98 (ddd, 2H), 8.22 (d, J=6.3 Hz, 1H), 8.53 (d, J=3.2 Hz, 1H). ¹⁹F NMR (376 MHz, CD₃OD): δ −115.78(q), −115.84(q), −134.36(t). Positive electrospray LC-MS, m/z 477 (M+H⁺). High resolution MS calculated for C₂₆H₂₀F₃N₄O₂ (M+H)=477.1533. Found 477.1496.

The following compounds were prepared using the above general procedure. The purified products were characterized by analytical reverse phase HPLC, LC-MS, ¹H NMR, ¹³C NMR, ¹⁹F NMR and HR-MS.

| Example | | Calculated | Found |
|---|---|---|---|
| No. | Compound Name(s) | (m + H) | (m + H) |
| 486 | 4-{(Z)-2-fluoro-2-[4-(4-oxo-4,5,6,7-tetrahydro- | 490.2249 | 490.2269 |
| | 1H-pyrrolo[3,2-c]pyridin-2-yl)pyridin-2- | | |
| | yl]vinyl}-N-(2-morpholin-4-ylethyl)benzamide | | |
| | trifluoroacetate | | |
| 487 | 4-{(Z)-2-fluoro-2-[4-(4-oxo-4,5,6,7-tetrahydro- | 421.167 | 421.1672 |
| | 1H-pyrrolo[3,2-c]pyridin-2-yl)pyridin-2- | | |
| | yl]vinyl}-N-(2-hydroxyethyl)benzamide | | |
| | trifluoroacetate | | |
| 488 | (2Z)-2-fluoro-N-{3-[5-fluoro-4-(4-oxo-4,5,6,7- | 471.1627 | 471.1605 |
| | tetrahydro-1H-pyrrolo[3,2-c]pyridin-2-yl)pyridin- | | |
| | 2-yl]phenyl}-3-phenylacrylamide trifluoroacetate | | |
| 489 | N-{3-[5-fluoro-4-(4-oxo-4,5,6,7-tetrahydro-1H- | 377.1408 | 377.1441 |
| | pyrrolo[3,2-c]pyridin-2-yl)pyridin-2- | | |
| | yl]phenyl}acrylamide trifluoroacetate | | |
| 485 | 2-fluoro-N-(3-fluorobenzyl)-4-[5-fluoro-4-(4-oxo- | 477.1533 | 477.1496 |
| | 4,5,6,7-tetrahydro-1H-pyrrolo[3,2-c]pyridin-2- | | |
| | yl)pyridin-2-yl]benzamide | | |
| 485 | 2-fluoro-N-(3-fluorobenzyl)-4-[5-fluoro-4-(4-oxo- | 477.1533 | 477 |
| | 4,5,6,7-tetrahydro-1H-pyrrolo[3,2-c]pyridin-2- | | |
| | yl)pyridin-2-yl]benzamide trifluoroacetate | | |
| 490 | N-(2-chlorobenzyl)-2-fluoro-4-[4-(4-oxo-4,5,6,7- | 475.1332 | 475.1335 |
| | tetrahydro-1H-pyrrolo[3,2-c]pyridin-2-yl)pyridin- | | |
| | 2-yl]benzamide trifluoroacetate | | |

### EXAMPLE 491

This example illustrates the preparation of 2-(2,6-dichloropyridin-4-yl)-1,5,6,7-tetrahydro-4H-pyrrolo[3,2-c]pyridin-4-one. The following procedure illustrates the general method for preparing the title compound.

Step 1: Preparation of 1-[2,6-dichloropyridin-4-yl]ethanone.

A 3 M MeMgCl solution in THF (50 mL, 150 mmol) was added dropwise over 25 min to a solution of 2,6-dichloronicotinic acid (9.6 g, 50 mmol) in THF (130 mL) at −45° C. and the reaction mixture was stirred at that temperature for another 15 min. The reaction mixture was allowed to warm to 0° C. and stirred at that temperature for another 1 h. The reaction mixture was recooled to −45° C., and methyl formate (6.2 mL, 100 mmo) was added dropwise to above solution. After 15 min at that temperature, 100 mL of 2 N HCl solution was added and the reaction mixture was allowed to warm to room temperature. The phases were separated and the lower aqueous layer was extracted with THF (2×20 ml). The combined organic layers were washed with a mixture of a saturated NaHCO₃ solution and a saturated NaCl solution and finally with a saturated solution of NaCl. The solvent was evaporated in vacuo, the residue was dissolved in CH₂Cl₂, dried with anhydrous MgSO₄ and evaporated in vacuo to afford a cream crystalline solid (7.85 g, 83%). ¹H NMR (300 MHz, CDCl₃): δ 2.63 (s, 3H), 7.68 (s, 2H). ¹³C NMR (75 MHz, CDCl₃): 626.85, 121.12, 147.63, 151.90, 194.26. GC-MS, m/z 189 (³⁵CIM+), 191 (³⁷CIM⁺).

Step 2: Preparation of 2-bromo-1-[2,6-dichloropyridin-4-yl]ethanone

2-Bromo-1-[2,6-dichloropyridin-4-yl]ethanone was prepared by the general procedure used for bromination of the 4-acetyl-2-chloropyridine. ¹H NMR (300 MHz, CDCl₃) δ 4.35 (s, 2H), 7.72 (s, 2H). Positive electrospray LC-MS, m/z 268, 270, 272 (M+H⁺) and m/z 286, 288, 290 (M+H₂O+H⁺)

Step 3: Preparation of 2-(2,6-dichloropyridin-4-yl)-1,5,6,7-tetrahydro-4H-pyrrolo[3,2-c]pyridin-4-one.

The compound was prepared using the general procedure used for the preparation of 2-(2-chloropyridin-4-yl)-1,5,6,7-tetrahydro-4H-pyrrolo[3,2-c]pyridin-4-one and was isolated as a cream solid. ¹H NMR (400 MHz, DMSO-d₆): δ 2.83 (t, J=6.8 Hz, 2H), 3.40 (dt, J=6.8 and 2.4 Hz, 2H), 7.14 (brs, 1H), 7.26 (d, J=2.3 Hz, 1H), 7.77 (s, 2H), 12.06 (brs, 1H). ¹³C NMR (100 MHz, DMSO-d₆): δ 21.75, 39.92, 109.54, 116.26, 126.24, 140.52, 144.93, 149.85, 164.36. Positive electrospray LC-MS, m/z 282 (³⁵CIM+H⁺), 284 (³⁷CIM+H⁺). High resolution MS calculated for C₁₂H₁₀C₁₂N₃O (M+H)=282.0195. Found 282.0147.

2-(2,6-dichloropyridin-4-yl)-1,5,6,7-tetrahydro-4H-pyrrolo[3,2-c]pyridin-4-one trifluoroacetate was prepared by dissolving the 2-(2,6-dichloropyridin-4-yl)-1,5,6,7-tetrahydro-4H-pyrrolo[3,2-c]pyridin-4-one in a mixture of DMF/water (1:1) containing a few drops of CF₃COOH. The crude residue was purified by reverse-phase C¹⁸ chromatography with a water/acetonitrile gradient containing 0.1% TFA. ¹H NMR (400 MHz, DMSO-d₆): δ 2.81 (t, J=7.0 Hz, 2H), 3.39 (t, J=7.0 Hz, 2H), 7.13 (brs, 1H), 7.26 (d, 1H), 7.78 (s, 2H), 12.07 (brs, 1H). ¹⁹FNMR (376 MHz, DMSO-d₆): δ −74.90. Positive electrospray LC-MS, m/z 282 (³⁵CIM+H⁺), 284 (³⁷CIM+H⁺).

Step 4: Preparation of 2-(2-aryl-6-chloropyridin-4-yl)-1,5,6,7-tetrahydro-4H-pyrrolo[3,2-c]pyridin-4-ones.

The following compounds were prepared using the general procedure of cross-coupling commercially available boronic acids with 2-(2,6-dichloropyridin-4-yl)-1,5,6,7-tetrahydro-4H-pyrrolo[3,2-c]pyridin-4-one. The purified products were characterized by analytical reverse phase HPLC, LC-MS, ¹H NMR, ¹³C NMR, ¹⁹F NMR and HR-MS.

| Example | | Calculated | Found |
|---|---|---|---|
| No. | Compound Name(s) | (m + H) | (m + H) |
| 491 | 2-(2,6-dichloropyridin-4-yl)-1,5,6,7-tetrahydro-4H- | 282.0195 | 282.0147 |
| | pyrrolo[3,2-c]pyridin-4-one | | |
| 491 | 2-(2,6-dichloropyridin-4-yl)-1,5,6,7-tetrahydro-4H- | 282.0195 | 282 |
| | pyrrolo[3,2-c]pyridin-4-one trifluoroacetate | | |
| 492 | 2-[2,6-bis(2-fluorophenyl)pyridin-4-yl]-1,5,6,7- | 402.1412 | 402.1383 |
| | tetrahydro-4H-pyrrolo[3,2-c]pyridin-4-one | | |
| | trifluoroacetate | | |
| 493 | 2-[2-chloro-6-(2-fluorophenyl)pyridin-4-yl]- | 342.0804 | 342.0792 |
| | 1,5,6,7-tetrahydro-4H-pyrrolo[3,2-c]pyridin-4-one | | |
| | trifluoroacetate | | |

### EXAMPLE 494

This example illustrates the preparation of 2-(2-amino-6-chloropyridin-4-yl)-1,5,6,7-tetrahydro-4H-pyrrolo[3,2-c]pyridin-4-one The following procedure illustrates the general method for preparing the title compound.

Step 1: Preparation of 2,6-dichloro-4-(2-methyl-1,3-dioxolan-2-yl)pyridine

A mixture of 1-(2,6-dichloropyridin-4-yl)ethanone (6.61 g, 34.8 mmol), ethylene glycol (50.0 mL, 896 mmol) and chlorotrimethylsilane was stirred at room temperature (rt) for 18 h. The reaction mixture was neutralized by the addition of 100 mL 1 N NaOH and extracted with 1:1 EtOAc/hexane mixture (4×100 mL). The combined organic extracts were dried with Na₂SO₄, and evaporated in vacuo to afford a colorless crystalline solid (8.45 g, 100%). ¹H NMR (400 MHz, CDCl₃): δ 1.60 (s, 3H), 3.78 (m, 2H), 4.07 (m, 2H), 7.36 (s, 2H). Positive electrospray LC-MS: m/z 234 (³⁵CIM+), 236 (³⁷CIM⁺).

Step 2: Preparation of 2-chloro-6-hydrazino-4-(2-methyl-1,3-dioxolan-2-yl)pyridine.

To a solution of 2,6-dichloro-4-(2-methyl-1,3-dioxolan-2-yl)pyridine in DMSO (6 mL) was added hydrazine hydrate (0.66 mL, 13.61 mmol) at rt and the reaction mixture was heated at 45° C. for 37 h. The reaction mixture was poured into 50 mL of water and the solid filtered, washed with water and dried in vacuo to afford a colorless crystalline solid (0.913 g). Analytical HPLC and ¹H NMR of the product showed 84% purity of the desired product. ¹H NMR (400 MHz, CDCl₃): δ 1.59 (s, 3H), 3.78 (m, 2H), 4.03 (m, 2H), 6.12 (brs, 1H), 6.73 (d, 1H), 6.77 (d, 1H). Positive electrospray LC-MS: m/z 230 (³⁵CIM⁺), 232 (³⁷CIM⁺).

Step 3: Preparation of 2-chloro-6-amino-4-(2-methyl-1,3-dioxolan-2-yl]pyridine.

A mixture of 2-chloro-6-hydrazino-4-(2-methyl-1,3-dioxolan-2-yl)pyridine (0.911 g, 4 mmol) and Raney-Nickel (150 mg) in 1-butanol (7 mL) was heated at 90° C. Hydrazine hydrate (0.39 mL, 8.0 mmol) was added dropwise at that temperature and the reaction mixture was heated for 20 min, cooled to rt, filtered and evaporated in vacuo to afford a colorless crystalline solid. The crude residue was purified by reverse-phase C¹⁸ chromatography with a water/acetonitrile containing 0.1% TFA gradient to afford a colorless crystalline solid (0.667 g). ¹H NMR (400 MHz, CDCl₃): δ 1.58 (s, 3H), 3.79 (m, 2H), 4.06 (m, 2H), 6.66 (d, J=1.2 Hz, 1H), 6.78 (d, J=1.2 Hz, 1H), 8.29 (brs, 2H). ¹³C NMR (100 MHz, CDCl₃): δ 26.46, 64.75, 104.61, 107.19, 109.97, 146.32, 158.13, 158.26. and 163.44 (q, TFA). ¹⁹FNMR (376 MHz, CDCl₃): δ −76.42. Positive electrospray LC-MS: m/z 215 (³⁵CIM+), 217 (³⁷CIM⁺). High resolution MS calculated for C₉H₁₂ClN₂O₂ (M+H)=215.0582. Found 215.0568.

Step 4: Preparation of 1-[2-amino-6-chloropyridin-4-yl]ethanone.

A solution of 2-chloro-6-amino-4-(2-methyl-1,3-dioxolan-2-yl]pyridine (0.730 g, 3.4 mmol) in 3 N HCl was stirred at rt for 3 days. The solvent was evaporated in vacuo to afford a yellow solid, triturated with CH₃CN and filtered to afford a yellow crystalline solid (0.537 g). ¹H NMR (400 MHz, CD₃OD): δ 2.51 (s, 3H), 4.85 (s, 4H), 6.90 (d, J=1.3 Hz, 1H), 6.93 (d, J=1.3 Hz, 1H 1H). Positive electrospray LC-MS: m/z 171 (³⁵CIM⁺), 173 (³⁷CIM⁺). High resolution MS calculated for C₇H₈ClN2O (M+H)=171.0320. Found 171.0347.

Step 5: Preparation of 1-[2-amino-6-chloropyridin-4-yl]-2-bromoethanone

1-[2-Amino-6-chloropyridin-4-yl]-2-bromoethanone was prepared by the general procedure used for bromination of the 4-acetyl-2-chloropyridine. ¹H NMR (400 MHz, CD₃CN): δ 4.55 (s, 2H), 5.43 (brs, 2H), 6.86 (d, J=1.2 Hz, 1H), 6.97 (d, J=1.2 Hz, 1H). Positive electrospray LC-MS, m/z 249, 251, 253 (M+H⁺) and m/z 267, 269, 271 (M+H₂O+H⁺)

Step 6: Preparation of 2-(2-amino-6-chloropyridin-4-yl)-1,5,6,7-tetrahydro-4H-pyrrolo[3,2-c]pyridin-4-one

The compound was prepared using the general procedure used for the preparation of 2-(2-chloropyridin-4-yl)-1,5,6,7-tetrahydro-4H-pyrrolo[3,2-c]pyridin-4-one and was isolated as a cream solid. ¹H NMR (400 MHz, CD₃OD): δ 1.89 (s, 2H), 2.91 (t, J=7 Hz, 2H), 3.55 (t, J=7.0 Hz, 2H), 6.61 (d, J=1.3 Hz, 1H), 6.82 (s, J=1.3 Hz, 1H), 6.90 (s, 1H). Positive electrospray LC-MS, m/z 263 (³⁵CIM+H⁺), 265 (³⁷CIM+H⁺). High resolution MS calculated for C₁₂H₁₂ClN₄O (M+H)=263.0694. Found 263.0693.

2-(2-Amino-6-chloropyridin-4-yl)-1,5,6,7-tetrahydro-4H-pyrrolo[3,2-c]pyridin-4-one trifluoroacetate was prepared by dissolving the 2-(2-amino-6-chloropyridin-4-yl)-1,5,6,7-tetrahydro-4H-pyrrolo[3,2-c]pyridin-4-one in a mixture of DMF/water (1:1) containing a few drops of CF₃COOH. The crude residue was purified by reverse-phase C¹⁸ chromatography with a water/acetonitrile gradient containing 0.1% TFA. ¹H NMR (400 MHz, CD₃OD): δ 2.93 (t, J=7.0 Hz, 2H), 3.56 (t, J=7.0 Hz, 2H), 6.72 (d, J=1.3 Hz, 1H), 6.98 (J=1.3 Hz, 1H), 7.03 (brs, 1H). ¹⁹FNMR (376 MHz, CD₃OD): δ −77.89. Positive electrospray LC-MS, m/z 263 (³⁵ClM+H⁺), 265 (³⁷ClM+H⁺).

Step 7: Preparation of 2-(2-aryl-6-aminopyridin-4-yl)-1,5,6,7-tetrahydro-4H-pyrrolo[3,2-c]pyridin-4-ones

The compounds were prepared using the general procedure by cross-coupling of the commercially available boronic acids with the 2-(2-amino-6-chloropyridin-4-yl)-1,5,6,7-tetrahydro-4H-pyrrolo[3,2-c]pyridin-4-one. The purified products were characterized by analytical reverse phase HPLC-MS, ¹H NMR, ¹³C NMR, ¹⁹F NMR and HR-MS.

| Example | | Calculated | Found |
|---|---|---|---|
| No. | Compound Name(s) | (m + H) | (m + H) |
| 494 | 2-(2-amino-6-chloropyridin-4-yl)-1,5,6,7-tetrahydro- | 263.0694 | 263.0693 |
| | 4H-pyrrolo[3,2-c]pyridin-4-one | | |
| 494 | 2-(2-amino-6-chloropyridin-4-yl)-1,5,6,7-tetrahydro- | 263.0694 | 263 (ES) |
| | 4H-pyrrolo[3,2-c]pyridin-4-one trifluoroacetate | | |
| 495 | 2-[2-amino-6-(2-fluorophenyl)pyridin-4-yl]- | 323.1303 | 323.131 |
| | 1,5,6,7-tetrahydro-4H-pyrrolo[3,2-c]pyridin- | | |
| | 4-one trifluoroacetate | | |
| 496 | 2-(2-amino-6-quinolin-3-ylpyridin-4-yl)-1,5,6,7- | 356.1506 | 356.1508 |
| | tetrahydro-4H-pyrrolo[3,2-c]pyridin-4-one | | |
| | trifluoroacetate | | |
| 497 | 2-{2-amino-6-[(E)-2-phenylvinyl]pyridin-4- | 331.1553 | 331.1587 |
| | yl}-1,5,6,7-tetrahydro-4H-pyrrolo[3,2- | | |
| | c]pyridin-4-one trifluoroacetate | | |

### EXAMPLE 498

This example illustrates the preparation of 2-[2-fluoro-6-(2-fluorophenyl)pyridin-4-yl]-1,5,6,7-tetrahydro-4H-pyrrolo[3,2-c]pyridin-4-one trifluoroacetate.

Step 1: Preparation of 2-(2-chloro-fluoropyridin-4-yl)-1,5,6,7-tetrahydro-4H-pyrrolo[3,2-c]pyridin-4-one.

A mixture of 2-(2,6-dichloropyridin-4-yl)-1,5,6,7-tetrahydro-4H-pyrrolo[3,2-c]pyridin-4-one and anhydrous potassium fluoride in sulfolane was heated at 200° C. under N₂ atm overnight. HPLC and LC-MS of the crude residue indicated the presence of the desired product as well as a byproduct: 2-(2,6-difluoropyridin-4-yl)-1,5,6,7-tetrahydro-4H-pyrrolo[3,2-c]pyridin-4-one. The crude reaction mixture was purified by reverse phase C¹⁸ HPLC with a water/acetonitrile gradient containing 0.1% TFA. The purified products were characterized by analytical reverse phase HPLC, LC-MS, ¹H NMR, ¹⁹F NMR and HR-MS. 2-(2-chloro-6-fluoropyridin-4-yl)-1,5,6,7-tetrahydro-4H-pyrrolo[3,2-c]pyridin-4-one high resolution MS calculated for C₁₂H₁₀ClFN₃O (M+H)=266.0491. Found 266.0473. 2-(2,6-difluoropyridin-4-yl)-1,5,6,7-tetrahydro-4H-pyrrolo[3,2-c]pyridin-4-one high resolution MS calculated for C₁₂H₁₀F₂N₃O (M+H)=250.0786. Found 250.0808.

Step2: Preparation of 2-[2-fluoro-6-(2-fluorophenyl)pyridin-4-yl]-1,5,6,7-tetrahydro-4H-pyrrolo[3,2-c]pyridin-4-one.

The compound was prepared using the general procedure by cross-coupling of the 2-fluorophenylboronic acid with the 2-(2-chloro-6-fluoropyridin-4-yl)-1,5,6,7-tetrahydro-4H-pyrrolo[3,2-c]pyridin-4-one. The purified product was characterized by analytical reverse phase HPLC, LC-MS, ¹H NMR, ¹⁹F NMR and HR-MS. High resolution MS calculated for C₁₈H₁₄F₂N₃O (M+H)=326.1099. Found 326.111.

### EXAMPLE 499

This example illustrates the preparation of 7-[2-(3-Fluorophenyl)pyridin-4-yl]-3,4-dihydropyrrolo[1,2-a]pyrazin-1(2H)-one hydrochloride.

Step 1: (Production of 7-bromo-3,4-dihydropyrrolo[1,2-a]pyrazin-1(2H)-one). The title compound was prepared from methyl 4-bromo-1H-pyrrole-2-carboxylate (prepared as described in J. Chem. Soc., Perkin Transactions 1, 10:1443-1447(1997)) as follows:

A solution of methyl 4-bromo-1H-pyrrole-2-carboxylate (2.18 g, 10 mmol) in 60 mL of DMF was cooled to −40° C. using a dry ice/acetonitrile bath. LiOₜBu (1M in THF)(12 mL, 12 mmol) was added drop-wise. The reaction mixture was stirred at that temperature for 1 hour. The N-Boc-amino ethyl bromide (2.69 g, 12 mmol) was taken up in 10 mL DMF and Nal (1.8 g, 12 mmol) was added. The mixture was stirred at 0° C. for 1 hour. This solution was then added dropwise to the above anion solution at −40° C. and kept stirred 1 hour. The reaction was then warmed to room temperature and kept stirred overnight. The reaction mixture was added water. The product precipitated was collected by filtration, washed with water and dried (1.74 g, 50.1%).

The above solid (1.74 g, 5 mmol) was dissolved in CH₂Cl₂ (10 mL) before TFA (5 mL) was added. The resultant mixture was stirred at room temperature for 1 hour, then, was blown dry with N₂. The residue was dissolved in ethanol, and NH₄OH was added. The mixture was stirred at room temperature for 15 minutes. Precipitation occurred. The solid was collected by filtration, washed with H₂O, rinsed with hexane, and air dried to give the title compound as an off white solid (650 mg, 60.3%): ¹HNMR (400 MHZ, DMSO-d6) δ 7.793 (s, 1H), 7.154(s, 1H), 6.639 (s, 1H), 4.075 (t, 2H), 4.490 (t, 2H), 3.494 (m, 2H);MS (EI) m/z: 215 (M+H).

Step 2: (Preparation of 2-(3-Fluorophenyl)-4-(4,4,5,5-tetramethyl-1,3,2-dioxaborolan-2-yl)pyridine).

To a slurry of 4-bromopyridine hydrochloride (2.21 g, 11.36 mmol) in THF (38 ml) was added 4-fluorophenyl magnesium bromide [4-Fluoro-1-bromobenzene (5.6 ml, 50.0 mmol) in THF (50 ml) with magnesium turnings (1.2 g, 50.0 mmol)] (25 ml, 25.0 mmol) at −78° C. The mixture was maintained at −78° C. for 20 minutes before phenylchloroformate (1.42 ml, 11.36 mmol) was added. The resultant slurry was maintained at −78° C. for an additional 20 minutes before warming to ambient temperature. The mixture was quenched with 10% ammonium chloride solution (50 ml). The layers were separated, aqueous layer was extracted with ether (3×40 ml). The organic washes were combined, extracted with water (1×20 ml), 1.0 N HCl (1×60 ml), brine (1×50 ml) and dried over Na₂SO₄. Concentration gave a yellow oil. The oil was suspended in toluene (57 ml). To this solution was added o-chloranil (2.79 g, 11.36 mmol) dissolved in glacial acetic acid (28 ml). The biphasic mixture was maintained at ambient temperature for one hour. The layers were separated. The aqueous layer was made basic with 50% NaOH (pH˜11). The layers were recombined and stirred well. The layers were separated again. The organic layer was washed with water (1×50 ml) and 2.0 N HCl (3×65 ml). The acidic layers were combined and basicified with 50% NaOH to pH˜11. The resultant basic solution was extracted with CH₂Cl₂ (3×65 ml). The CH₂Cl₂ washes were combined and dried over Na₂SO₄. Concentration gave 718.4 g of a yellow oil: ¹H NMR (400 MHz, MeOD) δ 8.46 (d, J=5.2 Hz, 1H), 8.10 (d, J=1.2 Hz, 1H), 7.80-7.73 (m, 2H), 7.56 (dd, J=5.6, 1.6 Hz, 1H), 7.48 (m, 1H), 7.18 (dt, J=8.4, 1.6 Hz, 1H); m/z 253 (M+H).

A mixture of triisopropyl borate (552 μl, 2.39 mmol) and the above compound (502.2 mg, 1.99 mmol) was suspended in toluene (3.2 ml) and THF (0.8 ml) at −78° C. To this solution was added n-BuLi 2.5 M in hexanes (0.95 ml), dropwise. The resultant mixture was maintained at −78° C. for 30 minutes before warming to −20° C. At −20° C., the mixture was quenched with 2.0 N HCl (2 ml). The biphasic mixture was warmed to ambient temperature and layers separated. The aqueous layer was neutralized to pH 7 using 2.5 N NaOH. The resultant solution was extracted with THF (3×10 ml). The THF extracts were combined and dried over Na₂SO₄. The oil contained a mixture of unreacted bromide and desired boronic acid. A flask charged with the oil mixture (396.5 mg, 1.83 mmol), pinacol (238.0 mg, 2.0 mmol) and toluene (10 ml) was fitted with a Dean-Stark trap and heated to 150-160° C. for 4 hours. The mixture was concentrated and purified by SiO₂ flash column chromatography eluting with CH₂Cl₂ to 1:19 methanol: CH₂Cl₂, which gave 268.9 mg of the title compound and unreacted pinacol: ¹H NMR (400 MHz, DMSO-d₆) δ 8.69 (dd, J=4.8, 0.8 Hz, 1H), 8.04 (s, 1H), 7.89 (d, J=8.0 Hz, 1H), 7.87-7.83 (m, 1H), 7.54 (dd, J=4.8, 0.8 Hz, 1H), 7.52-7.48 (m, 1H), 7.24 (dt, J=8.4, 2.8 Hz, 1H), 1.31 (s, 12H); m/z 299 (M).

Step 3: (Preparation of 7-[2-(3-Fluorophenyl)pyridin-4-yl]-3,4-dihydropyrrolo[1,2-a]pyrazin-1(2H)-one hydrochloride).

A slurry of the compound from Step 2 (268.9 mg, 0.90 mmol), compound from Step 1 (176.0 mg, 0.82 mmol), 2.0 M cesium carbonate solution (410 μl) and DMF (9 ml) was purged with nitrogen for 20 minutes before addition of tetrakistriphenylphosphine palladium (0) (76 mg). The slurry was purged with nitrogen for an additional 2-3 minutes then heated 80-90° C. overnight. Rotary evaporator removed excess solvent. The slurry was suspended in water and EtOAc. The aqueous layer was extract with EtOAc (3×10 ml); organic washes were combined and dried over Na₂SO₄. Purification was accomplished by SiO₂ flash column chromatography eluting with CH₂Cl₂ to 1:19 methanol: CH₂Cl₂, which gave desired product with triphenylphosphine oxide as an impurity. A second column was performed eluting with 1:49 methanol: CH₂Cl₂. The residue was dissolved in dioxane (5 ml) and 4.0 N HCl (5 ml) added and mixture stirred overnight. Solids were collected by filtration yielding 49.3 mg of the title compound: ¹H NMR (400 MHz, MeOD) δ 8.57 (d, J=6.4 Hz, 1H), 8.44 (d, J=2.0 Hz, 1H), 8.12 (dd, J=6.4, 1.6 Hz, 1H), 8.05 (d, J=2.0 Hz, 1H), 7.77 (d, J=7.6 Hz, 2H), 7.72 (m, 2H), 7.56 (d, J=1.6 Hz, 1H), 7.46 (m, 1H), 4.31 (m, 2H), 3.69 (m, 2H); m/z 308 (M+H).

### EXAMPLE 500

This example illustrates the preparation of 7-[2-(4-Methoxyphenyl)pyridin-4-yl]-3,4-dihydropyrrolo[1,2-a]pyrazin-1(2H)-one hydrochloride.

The title compound was prepared according to the procedure described for Example 499 for 7-[2-(3-Fluorophenyl)pyridin-4-yl]-3,4-dihydropyrrolo[1,2-a]pyrazin-1(2H)-one hydrochloride by using 4-methoxyphenyl magnesium bromide in Step 2: ¹H NMR (400 MHz, MeOD) δ 8.47 (d, J=6.4 Hz, 1H), 8.35 (d, J=2.0 Hz, 1H), 8.00 (m, 2H), 7.93 (m, 2H), 7.54 (d, J=1.6 Hz, 1H), 7.19 (d, J=8.8 Hz, 2H), 4.30 (m, 2H), 3.92 (s, 3H), 3.69 (m, 2H); m/z 320 (M+H).

### EXAMPLE 501

This example illustrates the preparation of 7-{2-[(E)-2-phenylethenyl]pyridin-4-yl}-3,4-dihydropyrrolo[1,2-a]pyrazin-1(2H)-one.

Step 1: (Production of 7-bromo-3,4-dihydropyrrolo[1,2-a]pyrazin-1(2H)-one). The title compound was prepared from methyl 4-bromo-1H-pyrrole-2-carboxylate (J. Chem. Soc., Perkin Transactions 1, 10:1443-1447(1997)) as described for Example 499.

Step2: (Preparation of 2-chloro-4-(4,4,5,5-tetramethyl-1,3,2-dioxaborolan-2-yl)pyridine). (TL,58(2002), 4369-4373).

To a slurry of N,N,N′,N′-tetramethylethylenediamine (3.32 ml, 22 mmol) in anhydrous diethyl ether(150 ml) cooled to −10° C. was added dropwise a 2.5M n-BuLi. The mixture was allowed to react at −10° C. for 20 minutes, then cooled to −60° C. A solution of 2-chloro-4-iodopyridine (4.79 g, 20 mmol) in ether (50 ml) was added dropwise, then allowed to react for 30 minutes while maintaining at −60° C. After cooing down to −70° C., a solution of triisopropyl borate (5.65 ml, 24 mmol) in ether (50 ml) was added in 10 minutes and allowed to react for 30 minutes before warmed to 10° C. (2 hours). A solution of anhydrous pinacol (3.14 g, 26 mmol) in ether (50 ml) was added and after 10 minutes, a solution of acetic acid (1.2 ml, 21 mmol) in ether(50 ml). The mixture was reacted for 4 hours, then filtered through celite, and extracted by 4% NaOH(200 ml). The aqueous layer was combined and acidified down to pH6 by dropwise addition of 3N HCl (˜90 ml) while keeping the internal temperature <5° C., then extracted with ether, dried over anhydrous Na₂SO₄, evaporated to obtain a yellow oil which solidified upon drying in vacuo (1.48 g, m/z: 240 (M+H) GC-MS) and used in next step.

Step3: (Preparation of 7-(2-chloro-pyridin-4-yl)-3,4-dihydro-2H-pyrrolo[1,2-a]pyrazin-1-one). Title compound was prepared from the products of Step1 and Step2 by the standard Suzuki coupling condition, (m/z: 248,(M+H), LC-MS) which was used in next step.

Step4: (Preparation of 7-(2-chloro-pyridin-4-yl)-3,4-dihydro-2H-pyrrolo[1,2-a]pyrazin-1-one). Title compound was prepared from the product of Step3 and commercially available trans-2-phenylvinylboronic acid by the standard Suzuki coupling condition, ¹HNMR (400 MHz, DMSO-d₆): δ 8.07(d, J=5.2 Hz, 1H), δ 7.81 (d, J=16 Hz, 1H), δ 7.66 (d, J=5.2 Hz, 1H), δ 7.55 (d, 2H), δ 7.38 (m, 3H), δ 7.10 (d, J=16 Hz, 1H), δ 7.17 (s, 1H) δ 7.05 (s, 1H), δ 6.77 (s, 1H), δ 4.09 (m, 2H), δ 3.37 (m, 2H); m/z: 316,(M+H).

### EXAMPLE 502

This example illustrates the preparation of 2-(2-chloropyridin-4-yl)-2,5,6,7-tetrahydro-4H-pyrrolo[3,4-c]pyridin-4-one trifluoroacetate.

Step 1. Synthesis of 3-[(dimethylamino)methylene]piperidine-2,4-dione. A suspension of piperdiene-2.4-dione (Example 1, step 3) (5.0 g, 44 mmol) in dimethylformamide dimethyl acetal was refluxed for 30 min. The reaction mixture was concentrated to dryness. The orange solid was suspended in ethyl acetate, filtered, and washed with ethyl acetate to give 3-[(dimethylamino)methylene]piperidine-2,4-dione as an orange solid (5.86 g, 34.8 mmol, 79% yield). LC-MS (ES+) MH⁺=169. ¹H NMR (400 MHz, DMSO-d₆) δ 7.82 (s, 1H), 7.13 (s, 1H), 3.28 (s, 3H), 3.15 (td, J=6.2, 3.3, 2H), 3.05 (s, 3H), 2.29 (t, J=6.3, 2H).

Step 2. Synthesis of 2,5-Diacetyl-2,5,6,7-tetrahydro-4H-pyrrolo[3,4-c]pyridin-4-one. A mixture of 3-[(dimethylamino)methylene]piperidine-2,4-dione (2.70 g, 16.1 mmol), glycine (1.27 g, 16.9 mmol), sodium acetate (1.39 g, 16.9 mmol), and ethanol (50 mL) was refluxed for 1 hour. The reaction was cooled to room temperature and filtered. The hydroscopic precipitate was washed with ethanol and dried under vacuum to give sodium N-[(2,4-dioxopiperidin-3-ylidene)methyl]glycinate as an off-white solid (2.84 g, 12.9 mmol, 80% yield). LC-MS (ES+) MH⁺=199.

A suspension of sodium N-[(2,4-dioxopiperidin-3-ylidene)methyl]glycinate (2.84 g, 12.9 mmol) in acetic anhydride (50 mL) and triethylamine (2.5 mL, 17.7 mmol) was refluxed for 30 min. The reaction was concentrated and purified by flash chromatography (30% ethyl acetate/hexanes) to give 2,5-diacetyl-2,5,6,7-tetrahydro-4H-pyrrolo[3,4-c]pyridin-4-one as a white solid (1.05 g, 4.77 mmol, 37% yield). LC-MS (ES+) MH⁺=221. ¹H NMR (300 MHz, CDCl₃) δ 7.88 (d, J=2.2, 1H), 7.14 (s, 1H), 4.09 (t, J=6.1, 2H), 2.74 (t, J=5.7, 2H), 2.58 (s, 3H), 2.56 (s, 3H).

Step 3. Synthesis of 2,5,6,7-tetrahydro-4H-pyrrolo[3,4-c]pyridin-4-one. A suspension of 2,5-diacetyl-2,5,6,7-tetrahydro-4H-pyrrolo[3,4-c]pyridin-4-one (1.05 g, 4.77 mmol) in methanol was treated with 0.5 M sodium methoxide in methanol (19.1 mL, 9.54 mmol). The reaction was stirred at room temperature for 30 minutes. The solution was concentrated to give an oil which was dissolved in tetrahydrofuran and treated with 10 mL of 1 N HCl in diethyl ether. The sodium chloride salt was filtered away, and the filtrate was concentrated to give 2,5,6,7-tetrahydro-4H-pyrrolo[3,4-c]pyridin-4-one as a yellow-orange solid (604 mg, 4.44 mmol, 93% yield). LC-MS (ES+) MH⁺=137. ¹H NMR (300 MHz, DMSO-d₆) δ 11.05 (br s, 1H), 7.10 (br s, 2H), 6.56 (s, 1H), 3.27 (t, J=6.4, 2H), 2.60 (t, J=6.3, 2H).

Step 4. Synthesis of 2-(2-chloropyridin-4-yl)-2,5,6,7-tetrahydro-4H-pyrrolo[3,4-c]pyridin-4-one. A mixture of 2,5,6,7-tetrahydro-4H-pyrrolo[3,4-c]pyridin-4-one (537 mg, 3.94 mmol), 2-chloro-4-iodopyridine (944 mg, 3.94 mmol), copper(I) iodide (75 mg, 0.394 mmol), potassium phosphate (1.76 g, 8.27 mmol), trans-1,2-diaminocyclohexane (45 mg, 0.394 mmol) and dioxane (10 mL) was refluxed under nitrogen for 78 hours. The reaction mixture was cooled to room temperature and poured onto a 50 g silica gel column. The solvent was allowed to evaporate under a stream of nitrogen. The product was eluted with 70→100% ethyl acetate/hexanes then 0→10% methanol/ethylacetate to give 2-(2-chloropyridin-4-yl)-2,5,6,7-tetrahydro-4H-pyrrolo[3,4-c]pyridin-4-one as an off-white solid (317 mg, 1.28 mmol, 32% yield. LC-MS (ES+) MH⁺=248. ¹H NMR (400 MHz, DMSO-d₆) δ 8.39 (d, J=5.7, 1H), 8.10 (d, J=2.1, 1H), 7.94 (d, J=2.1, 1H), 7.78 (dd, J=5.9, 2.1, 1H), 7.54 (s, 1H), 7.46 (s, 1H), 3.33 (td, J=6.5, 2.5, 2H), 2.68 (t, J=6.5, 2H).

Step 5: 2-(2-chloropyridin-4-yl)-2,5,6,7-tetrahydro-4H-pyrrolo[3,4-c]pyridin-4-one trifluoroacetate.

Purification of 2-(2-chloropyridin-4-yl)-2,5,6,7-tetrahydro-4H-pyrrolo[3,4-c]pyridin-4-one by reverse-phase HPLC (acetonitrile/water/0.05% trifluoroacetic acid gave the trifluoroacetate salt. ¹H NMR (300 MHz, DMSO-d₆) δ 8.39 (d, J=5.6, 1H), 8.11 (d, J=2.0, 1H), 7.94 (d, J=1.8, 1H), 7.79 (dd, J=5.7, 2.0, 1H), 7.54 (d, J=1.0, 1H), 7.46 (s, 1H), 3.32 (t, J=6.3, 2H), 2.68 (t, J=6.3, 2H). HRMS calculated for C₁₂H₁₁N₃OCl (MH⁺) 248.0585, found 248.0579.

### EXAMPLE 503

This example illustrates the preparation of 2-(2-quinolin-3-ylpyridin-4-yl)-2,5,6,7-tetrahydro-4H-pyrrolo[3,4-c]pyridin-4-one bis(trifluoroacetate).

The title compound was prepared from 2-(2-chloropyridin-4-yl)-2,5,6,7-tetrahydro-4H-pyrrolo[3,4-c]pyridin-4-one and 3-quinolinylboronic acid by the procedure described for Example 2. ¹H NMR (300 MHz, DMSO-d₆) δ 9.80 (d, J=2.2, 1H), 9.34 (d, J=1.8, 1H), 8.76 (d, J=5.6, 1H), 8.55 (d, J=2.0, 1H), 8.32 (d, J=2.2, 1H), 8.16 (d, J=8.5, 1H), 8.13 (d, J=9.0, 1H), 7.89 (td, J=7.0, 1.5, 1H), 7.82 (dd, J=5.6, 2,2, 1H), 7.74 (td, J=7.5, 1.1, 1H), 7.67 (d, J=2.0, 1H), 7.46 (s, 1H), 3.37 (t, J=6.2, 2H), 2.74 (t, J=6.2, 2H). HRMS calculated for C₂₁H₁₇N₄O (MH⁺) 341.1397, found 341.1386.

### EXAMPLE 504

This example illustrates the preparation of 2-[2-(2-fluorophenyl)pyridin-4-yl]-2,5,6,7-tetrahydro-4H-pyrrolo[3,4-c]pyridin-4-one trifluoroacetate.

The title compound was prepared from 2-(2-chloropyridin-4-yl)-2,5,6,7-tetrahydro-4H-pyrrolo[3,4-c]pyridin-4-one and 2-fluorophenylboronic acid by the procedure described for Example 2. ¹H NMR (300 MHz, DMSO-d₆) δ 8.74 (d, J=5.8, 1H), 8.13 (d, J=1.8, 1H), 8.10 (s, 1H), 7.91 (td, J=8.0, 1.3, 1H), 7.84 (dd, J=5.7, 2.1, 1H), 7.61-7.51 (m, 2H), 7.47 (s, 1H), 7.43-7.33 (m, 2H), 3.34 (t, J=5.6, 2H), 2.70 (t, J=6.1, 2H). HRMS calculated for C₁₈H₁₅FN₃O (MH⁺) 308.1194, found 308.1205.

### EXAMPLE 505

This example illustrates the preparation of 2-{2-[(E)-2-phenylethenyl]pyridin-4-yl}-1,4,5,6-tetrahydro-7H-pyrrolo[2,3-c]pyridin-7-one

Step 1: A mixture of 2-chloro-4-bromoacetylpyridine (1.98 g, 8.5 mmol), 2,3-dioxopiperidine (0.80 g, 7.1 mmol), and ammonium acetate (2.73 g, 7.1 mmol) in EtOH (20 mL) was stirred for 4 h at ambient temperature. The resulting mixture was concentrated, dissolved in CH₂Cl₂ and washed with saturated NaHCO₃, followed with brine. The organic solution was dried (MgSO₄), concentrated, and purified by C18 reverse phase HPLC to afford 2-(2-chloropyridin-4-yl)-1,4,5,6-tetrahydro-7H-pyrrolo[2,3-c]pyridin-7-one as a light brown solid. ¹H NMR (CD₃OD) δ 2.77 (t, J=7 Hz, 2H), 3.48 (m, 2H), 6.71 (s, 1H), 7.60 (dd, 1H), 7.73 (s, 1H), 8.25 (d, J=6 Hz, 1H). High resolution MS calculated for C₁₂H₁₁ClN₃O (M+H)=248.0585. Found 248.0611.

Step 2: The title compound was prepared 2-(2-chloropyridin-4-yl)-1,4,5,6-tetrahydro-7H-pyrrolo[2,3-c]pyridin-7-one by the method described for Example 2. ¹H NMR (CD₃OD) δ 2.77 (t, J=7 Hz, 2H), 3.48 (m, 2H), 6.71 (s, 1H), 7.60 (dd, 1H), 7.73 (s, 1H), 8.25 (d, J=6 Hz, 1H). High resolution MS calculated for C₂₀H₁₈N₃O (M+H)=316.1444. Found 316.1447.

The following examples were prepared by the same method described for Example 505:

### EXAMPLE 506

This example illustrates the preparation of 2-[2-(2-fluorophenyl)pyridin-4-yl]-1,4,5,6-tetrahydro-7H-pyrrolo[2,3-c]pyridin-7-one.

¹H NMR (CD₃OD) δ 2.77 (t, J=7 Hz, 2H), 3.48 (t, J=7 Hz, 2H), 7.03 (s, 1H), 7.17 (d, J=6 Hz, 1H), 7.40 (m, 3H), 7.61 (m, 2H), 7.81 (s, 1H), 7.86 (t, J=6 Hz, 1H), 8.39 (m, 2H). High resolution MS calculated for C₁₈H₁₅FN₃O (M+H)=308.1194. Found 308.1211.

### EXAMPLE 507

This example illustrates the preparation of 2-(2-phenylpyridin-4-yl)-1,4,5,6-tetrahydro-7H-pyrrolo[2,3-c]pyridin-7-one.

¹H NMR (CD₃OD) δ 2.82 (t, J=7 Hz, 2H), 3.52 (t, J=7 Hz, 2H) 7.09 (s, 1H), 7.62 (m, 2H), 7.93 (m, 2H), 8.02 (dd, 1H), 8.44 (d, J=2 Hz, 1H), 8.55 (d, J=6 Hz, 1H).). High resolution MS calculated for C₁₈H₁₆N₃O (M+H)=290.1288. Found 290.1318.

### EXAMPLES 508-511

Reserved.

### EXAMPLE 512

This example illustrates the preparation of 2-{2-[(E)-2-phenylethenyl]pyridin-4-yl}-1,5,6,7-tetrahydro-4H-imidazo[4,5-c]pyridin-4-one.

Step 1: While maintaining an internal reaction temperature of 10° C., isopropylnitrite (5.90 g, 66 mmol) was added dropwise over 1 hr to an ice-water cooled solution of 2,4-dioxopiperidine (5.00 g, 44 mmol) in 1M ethanolic HCl (13 mL). After stirring an additional 30 min at ambient temperature, the mixture was diluted with water (10 mL) and the precipitate was collected by filtration. This solid was washed several times with water and ether, and dried under vacuum to give (3Z)-piperidine-2,3,4-trione 3-oxime hydrochloride as an off-white solid (4.37 g, 70%).

Step 2: 2-Chloro-4-cyanopyridine (5.00 g, 0.036 mol) in 0.5 M ammonia in isopropanol (90 mL) was hydrogenated at 70 psi over active chromium promoted cobalt catalyst in water (Raney 2724 from Davison Chemical, 0.5 g) at ambient temperature for 3 days. The resulting reaction mixture was filtered to remove the catalyst, then concentrated, dissolved in CH₂Cl₂ and extracted several times with 0.1% TFA in H₂0. The aqueous extracts were combined and evaporated to give 2.50 g of the trifluoroacetic acid salt of 2-chloro-4-aminomethylpyridine as an off-white solid.

Step 3: A mixture of (3Z)-piperidine-2,3,4-trione 3-oxime hydrochloride (2.14 g, 15 mmol) and the trifluoroacetic acid salt of 2-chloro-4-aminomethylpyridine (2.35 g, 16.56 mmol) in DMSO (30 mL) was stirred at 80° C. for 4 hours. The reaction mixture was concentrated under reduced pressure, then was re-dissolved in 0.1% TFA in water and washed several times with CH₂Cl₂. The aqueous layer was concentrated under reduced pressure and purified by C-18 reversed HPLC to afford 1.20 g of 2-(2-chloropyridin-4-yl)-1,5,6,7-tetrahydro-4H-imidazo[4,5-c]pyridin-4-one as a pale-yellow solid. ¹H NMR (CDCl₃) δ 3.09 (t, J=7 Hz, 2H), 3.79 (m, 2H), 5.99 (s, 1H), 7.91 (m, 1H), 8.15 (s, 1H), 8.49 (d, J=5 Hz, 1H). High resolution MS calculated for C₁₁H₉ClN₄O (M+H)=248.6682. Found 248.6685.

Step 4: The title compound was prepared from 2-(2-chloropyridin-4-yl)-1,5,6,7-tetrahydro-4H-imidazo[4,5-c]pyridin-4-one by the method described for Example 2. ¹H NMR (DMSO) δ 2.84 (4, J=7 Hz, 2H), 3.43 (m, 2H), 7.34 (d, J=2 Hz, 1H), 7.35 (s, 1H), 7.41 (m, 2H), 7.50 (s, 1H), 7.65 (m, 2H), 7.78 (d, J=6 Hz, 1H), 7.93 (dd, 1H), 8.38 (s, 1H), 8.66 (d, J=6 Hz, 1H). High resolution MS calculated for C₁₉H₁₇N₄O (M+H)=317.1397. Found 317.1399.

The following examples were prepared by the same method described for Example 512.

### EXAMPLE 513

This example illustrates the preparation of 2-(2-quinolin-3-ylpyridin-4-yl)-1,5,6,7-tetrahydro-4H-imidazo[4,5-c]pyridin-4-one.

¹H NMR (DMSO) δ 2.83 (m, 2H), 3.44 (m, 2H), 7.46 (m, 1H), 7.63 (t, J=7 Hz, 1H), 7.78 (td, 1H), 7.95 (s, 1H), 8.04 (d, J=8 Hz, 1H), 8.11 (d, J=8 Hz, 1H), 8.77 (s, 1H), 8.78 (s, 1H), 9.02 (d, J=2 Hz, 1H), 9.63 (d, J=2 Hz, 1H). High resolution MS calculated for C₂₀H₁₆N₅O (M+H)=342.1349. Found 342.1367.

### EXAMPLE 514

This example illustrates the preparation of 2-(2-pyridin-3-ylpyridin-4-yl)-1,5,6,7-tetrahydro-4H-imidazo[4,5-c]pyridin-4-one.

¹H NMR (DMSO) δ 2.82 (t, J=7 Hz, 2H), 3.43 (m, 2H), 7.49 (s, 1H), 7.52 (dd, 1H), 7.92 (dd, 1H), 8.33 (s, 1H), 8.43 (d, J=8 Hz, 1H), 8.61 (m, 2H), 8.72 (d, J=5 Hz, 1H), 9.27 (d, J=2 Hz, 1H). High resolution MS calculated for C₁₆H₁₄N₅O (M+H)=292.1193. Found 292.1174.

### EXAMPLE 515

This example illustrates the preparation of 7-[2-(3-fluorophenyl)pyridin-4-yl]-3,4,5,6-tetrahydro-2H-pyrrolo[2,3-f][1,2]thiazepine 1,1-dioxide trifluoroacetate.

Step 1. Synthesis of 5-(2-chloropyridin-4-yl)-1H-pyrrole-3-sulfonic acid. Concentrated sulfuric acid was added to 2-chloro-4-(1H-pyrrol-2-yl)pyridine (5.0 g, 28 mmol) at room temperature with stirring. The reaction was exothermic as the temperature rose to 55° C. After 30 minutes, the dark syrup was poured into 250 mL ice water with stirring. The precipitate was filtered, washed with water and diethyl ether, and dried under vacuum to give 5-(2-chloropyridin-4-yl)-1H-pyrrole-3-sulfonic acid as an orange solid (4.91 g, 19.0 mmol, 68% yield). The position of the sulfonic acid was confirmed by ¹H NMR NOE experiments. LC-MS (ES+) MH⁺=259. ¹H NMR (300 MHz, DMSO-d₆) δ 11.70 (s, 1H), 8.25 (d, J=5.4, 1H), 7.73 (d, J=1.0, 1H), 7.61 (dd, J=5.3, 1.5, 1H), 7.07 (dd, J=2.7, 1.5, 1H), 6.95 (dd, J=2.5, 1.5, 1H), 6.36 (br s, 1H).

Step 2. Synthesis of ethyl N-{[5-(2-chloropyridin-4-yl)-1H-pyrrol-3-yl]sulfonyl}-beta-alaninate. A suspension of 5-(2-chloropyridin-4-yl)-1H-pyrrole-3-sulfonic acid (4.13 g, 16.0 mmol) in methanol (65 mL) was treated with 0.5 M sodium methoxide (32 mL, 16.0 mmol) to give the sodium 5-(2-chloropyridin-4-yl)-1H-pyrrole-3-sulfonate (4.6 g).

A solution of sodium 5-(2-chloropyridin-4-yl)-1H-pyrrole-3-sulfonate (1.0 g, 3.56 mmol) in dimethylformamide (20 mL) at 0° C. was slowly treated with thionyl chloride (0.780 mL, 10.7 mmol). After 2 hours at 0° C., beta-alanine ethyl ester hydrochloride (2.7 g, 17.8 mmol) was added portionwise, followed by triethylamine (5.0 mL, 35.6 mmol). After stirring for 3 hours at 0° C., the reaction was allowed to warm to room temperature overnight. The reaction mixture was poured into half-saturated ammonium chloride and was extracted with ethyl acetate. The organic layers were washed with 0.5 N HCl and brine, dried (sodium sulfate), concentrated, and purified by flash chromatography (1→10% methanol/dichloromethane) to give the title compound as an off-white solid (650 mg, 1.82 mmol, 51% yield). LC-MS (ES+) MH⁺=358. ¹H NMR (300 MHz, DMSO-d₆) δ 12.46 (s, 1H), 8.35 (d, J=5.4, 1H), 7.84 (d, J=1.0, 1H), 7.70 (dd, J=5.3, 1.5, 1H), 7.54 (d, J=2.9, 1.6, 1H), 7.26 (t, J=5.9, 1H), 7.19 (dd, J=2.5, 1.6, 1H), 4.02 (q, J=7.0, 2H), 3.06-2.98 (m, 2H), 2.45 (t, J=7.1, 2H), 1.14 (t, J=7.0, 3H).

Step 3. Synthesis of N-({5-[2-(3-fluorophenyl)pyridin-4-yl]-1H-pyrrol-3-yl}sulfonyl)-beta-alanine trifluoroacetate. A mixture of ethyl N-{[5-(2-chloropyridin-4-yl)-1H-pyrrol-3-yl]sulfonyl}-beta-alaninate (610 mg, 1.70 mmol), 3-fluorophenylboronic acid (360 mg, 2.56 mmol), tetrakis(triphenylphospine)palladium(0) (98 mg, 0.085 mmol), 2 M cesium carbonate (2.6 mL, 5.1 mmol), ethanol (2 mL), and dimethylformamide (12 mL) was stirred at 80° C. overnight. The reaction mixture was cooled, acidified with aq. HCl and TFA, filtered through a syringe filter, and purified by reverse-phase HPLC (acetonitrile/water/0.05% trifluoroacetic acid) to give the title compound as an off-white solid (487 mg, 0.968 mmol, 57% yield). LC-MS (ES+) MH⁺=390. ¹H NMR (300 MHz, DMSO-d₆) δ 12.50 (s, 1H), 8.65 (d, J=5.3, 1H), 8.38 (d, J=0.9, 1H), 8.05-7.95 (m, 2H), 7.75 (dd, J=5.4, 1.6, 1H), 7.63-7.54 (m, 2H), 7.37-7.29 (m, 2H), 7.20 (t, J=5.9), 3.01 (td, J=7.1, 5.6, 2H), 2.41 (t, J=7.1, 2H).

Step 4. Synthesis of 7-[2-(3-fluorophenyl)pyridin-4-yl]-3,4,5,6-tetrahydro-2H-pyrrolo[2,3-f][1,2]thiazepine 1,1-dioxide trifluoroacetate.

A suspension of N-({5-[2-(3-fluorophenyl)pyridin-4-yl]-1H-pyrrol-3-yl}sulfonyl)-beta-alanine trifluoroacetate (425 mg, 0.844 mmol) in polyphosphoric acid was stirred mechanically at 80° C. for 3.5 hours. The syrup was poured into 200 mL of water. The suspension was neutralized with aqueous sodium hydroxide and extracted with ethyl acetate (200 mL). The organic layer was washed with water and brine, dried (sodium sulfate), and concentrated to give 7-[2-(3-fluorophenyl)pyridin-4-yl]-3,4-dihydro-2H-pyrrolo[2,3-f][1,2]thiazepin-5(6H)-one 1,1-dioxide as a crude solid (159 mg). The solid was dissolved in trifluoracetic acid (3.0 mL) and treated with triethylsilane (0.50 mL, 3.13 mmol) at room temperature. The reaction was stirred at room temperature for 4 days. The solution was concentrated and purified by reverse-phase HPLC (acetonitrile/water/0.05% trifluoroacetic acid) to give the title compound as a yellow solid (58 mg, 0.123 mmol, 15% yield). The regiochemistry was confirmed by ¹H NMR NOE experiments. ¹H NMR (300 MHz, DMSO-d₆) δ 12.14 (s, 1H), 8.63 (d, J=5.7, 1H), 8.34 (d, J=1.1, 1H), 8.03-7.94 (m, 2H), 7.75 (dd, J=5.5, 1.3, 1H), 7.60 (td, J=8.2, 6.2, 1H), 7.40 (d, J=2.5, 1H), 7.39-7.26 (m, 2H), 3.45-3.35 (m, 2H), 3.05-2.98 (m, 2H), 1.85-1.75 (m, 2H). HRMS calculated for C₁₈H₁₇FN₃O₂S (MH⁺) 358.1020, found 358.1058.

### EXAMPLE 516

This example illustrates the preparation of 2-[1-(4-fluorophenyl)-5-methyl-1H-pyrazol-4-yl]-1,5,6,7-tetrahydro-4H-pyrrolo[3,2-c]pyridin-4-one trifluoroacetate.

Step 1. (Preparation of 3-[(dimethylamino)methylene]pentane-2,4-dione).

A solution of 2,4-pentanedione (1.0 g, 10.2 mmol) in 20 mL of dimethylformamide dimethyl acetal was heated to reflux for 18 hours, cooled to room temperature and condensed to give the title compound (1.57 g, 10.1 mmol, 98%) m/z (M+H): 156.

Step 2. (Preparation of 1-[1-(4-fluorophenyl)-5-methyl-1H-pyrazol-4-yl]ethanone).

A solution of 3-[(dimethylamino)methylene]pentane-2,4-dione (800 mg, 5.1 mmol) and 4-fluorophenyl hydrazine hydrochloride (1.0 g, 602 mmol) in 15 mL of methanol was treated with 5.0 mL of 2.0 M potassium hydroxide solution and heated to 65 degrees celcius for 18 hours. The reaction was cooled to room temperature and poured into water and extracted with ethyl acetate washed with brine, dried over magnesium sulfate filtered and condensed. Purified by flash column chromatography (gradient: 10% ethyl acetate/hexanes to 65% ethyl acetate/hexanes) to give the title compound as an orange solid (800 mg, 3.6 mmol, 60%) m/z (M+H): 219

Step 3. (Preparation of 2-bromo-1-[1-(4-fluorophenyl)-5-methyl-1H-pyrazol-4-yl]ethanone).

A solution of 1-[1-(4-fluorophenyl)-5-methyl-1H-pyrazol-4-yl]ethanone (800 mg, 3.6 mmol) in 15 mL of acetic acid was treated with bromine (0.22 mL, 4.4 mmol) and 0.87 mL of a 33% hydrobromic acid in acetic acid solution, stirred 3 hours and condensed. Water and saturate sodium bicarbonate were added and the solution extracted three times with ethyl acetate, washed with brine, dried over magnesium sulfate, filtered and condensed. Purification by flash column chromatography (gradient: 10% ethyl acetate/hexanes to 65% ethyl acetate/hexanes) gave the title compound as a orange waxy solid (700 mg, 2.3 mmol, 65%) m/z (M+H): 297/299.

Step 4. (Preparation of 2-[1-(4-fluorophenyl)-5-methyl-1H-pyrazol-4-yl]-1,5,6,7-tetrahydro-4H-pyrrolo[3,2-c]pyridin-4-one trifluoroacetate).

A solution of 2-bromo-1-[1-(4-fluorophenyl)-5-methyl-1H-pyrazol-4-yl]ethanone (650 mg, 2.2 mmol) and 2,4-dioxopiperdine (295 mg, 2.6 mL) in 10 mL of DME was cooled to zero degrees celcius and treated with 4.6 mL of potassium tert-butoxide 1.0 M in tert-butanol solution and allowed to warm to room temperature and condensed to dryness. The residue was dissolved in 10 mL of ethyl alcohol and treated with ammonium acetate (850 mg, 11.0 mmol) and heated to reflux for six hours, cooled to room temperature and condensed. 4 mL of 50% acetonitrile/water was added to the residue along with 1.0 mL of trifluoroacetic acid, filtered throught a syringe filter (0.45 μm) purified by rpHPLC, and lyopholized to give the title compound as an off-white solid (110 mg, 0.26 mmol, 11%). ¹H NMR (400 MHz, DMSO-d₆) δ 11.33 (s, 1H), 7.81 (s, 1H), 7.58-7.52 (m, 2H), 7.35 (t, J=8.7 Hz, 2H), 6.88 (bs, 1H), 6.23 (d, J=2.8 Hz, 1H), 3.36 (t, J=7.0 Hz, 2H), 2.77 (t, J=6.8 Hz, 2H), 2.34 (s, 3H). HRMS calculated for C₁₇H₁₅FN₄O (MH⁺) 311.1303, found 311.1299. Anal. calculated for C₁₇H₁₅FN₄O.1.0 TFA.0.1 H₂O C, 53.55; H, 3.83; N, 13.14. Found: C, 53.51; H, 3.82; N, 13.16.

### EXAMPLE 517

This example illustrates the preparation of 2-[2-(4-bromophenyl)pyrimidin-4-yl]-1,5,6,7-tetrahydro-4H-pyrrolo[3,2-c]pyridin-4-one trifluoroacetate.

Step 1. (4-bromobenzenecarboximidamide).

To a solution of 4-bromobenzonitrile (5.02 g, 27.6 mmol) in 33 ml of THF was added a 1 M solution of Lithium bis(trimethylsilyl)amide (33.1 ml) in tetrahydrofuran. The resulting solution was stirred for 1 hour. The solution was cooled on an ice bath and the reaction was quenched by dropwise addition of 27.6 ml of 3 M HCl(aq). The mixture was stirred for 30 min at room temperature and the organic layer was discarded. The aqueous layer was treated with ethyl acetate (100 ml) and made basic by addition of 2.5 N NaOH. The layers were separated and the aqueous layer was extracted 3 times with 50 ml of ethyl acetate. The combined organic layers were dried over sodium sulfate, filtered and evaporated to give 4-bromobenzenecarboximidamide (3.84 g, 70%) as a white solid. m/z (M+H): 199

Step 2. (Preparation of (1E)-1-(dimethylamino)-4,4-dimethoxypent-1-en-3-one)

(1E)-1-(dimethylamino)-4,4-dimethoxypent-1-en-3-one was prepared by a literature method (Lipinski, C. A., et al., J. Heterocyclic Chem., 22:1723 (1985)). from 3,3-dimethoxy-butan-2-one and dimethylformamide dimethylacetal purchased from Sigma-Aldrich Corporation.

Step 3. (Preparation of 2-(4-bromophenyl)-4-(1,1-dimethoxyethyl)pyrimidine).

A solution of 4-bromobenzenecarboximidamide (1.09 g, 5.48 mmol) and (1E)-1-(dimethylamino)-4,4-dimethoxypent-1-en-3-one (1.02 g, 5.47 mmol) in 10 ml of ethanol was refluxed for 20 hours. The solution was concentrated in vacuo and the residue was dissolved in dichloromethane and purified by flash chromatography (10-30% ethyl acetate/hexanes) to give 2-(4-bromophenyl)-4-(1,1-dimethoxyethyl)pyrimidine (1.44 g, 81%) as a white solid. m/z (M+H): 323

Step 4. (Preparation of 1-[2-(4-bromophenyl)pyrimidin-4-yl]ethanone trifluoroacetate).

To a solution of 2-(4-bromophenyl)-4-(1,1-dimethoxyethyl)pyrimidine (1.44 g, 4.46 mmol) in 20 ml of chloroform was added 10 ml of a 50:50 mixture(v/v) of trifluoroacetic acid in water. The resulting mixture was vigorously stirred for 2 hours. The mixture was concentrated in vacuo. Toluene was added to the oil and the mixture was concentrated in vacuo to azeotrope off the water (repeat 2 times) to give 1-[2-(4-bromophenyl)pyrimidin-4-yl]ethanone trifluoroacetate (1.18 g, 95%) as a white solid. m/z (M+H): 277

Step 5. (Preparation of 2-bromo-1-[2-(4-bromophenyl)pyrimidin-4-yl]ethanone).

To a solution of 1-[2-(4-bromophenyl)pyrimidin-4-yl]ethanone trifluoroacetate (1.17 g, 4.22 mmol) in tetrahydrofuran (64 ml) was added tetrabutylammonium tribromide (2.03 g, 4.22 mmol) and the solution was stirred overnight. The solution was concentrated in vacuo and purified by flash chromatography (20-50% ethyl acetate/hexanes) to give 2-bromo-1-[2-(4-bromophenyl)pyrimidin-4-yl]ethanone (1.42 g, 95%) as a white solid. m/z (M+H): 355

Step 6. (Preparation of 2-[2-(4-bromophenyl)pyrimidin-4-yl]-1,5,6,7-tetrahydro-4H-pyrrolo[3,2-c]pyridin-4-one trifluoroacetate).

2-Bromo-1-[2-(4-bromophenyl)pyrimidin-4-yl]ethanone (0.82 g, 2.31 mmol) was combined in absolute ethanol (23 mL) with ammonium acetate (0.71 g, 9.24 mmol) and 2,4 dioxopiperdine (0.26 g, 2.31 mmol) and stirred overnight. The resulting precipitate was collected by vacuum filtration. The solid was purified by reverse-phase HPLC (30-60% acetonitrile/water/0.05% trifluoroacetic acid) to give 2-[2-(4-bromophenyl)pyrimidin-4-yl]-1,5,6,7-tetrahydro-4H-pyrrolo[3,2-c]pyridin-4-one trifluoroacetate (0.27 g, 32%) as a yellow solid.

¹H NMR (400 MHz, DMSO-d₆) δ 12.15 (s, 1H), 8.72 (d, J=5.4 Hz, 1H), 8.55 (m, 2H), 7.74 (m, 2H), 7.57 (m, 1H), 7.34 (d, J=5.4 Hz, 1H), 7.16 (s, 1H), 3.44 (t, J=6.8 Hz, 2H), 2.93 (t, J=6.5 Hz, 2H). HRMS calculated for C₁₇H₁₄BrN₄O (MH⁺) 369.0345, found 369.0326. Anal. calculated for C₁₇H₁₄BrN₄O.0.65 TFA.1.55 H₂O C, 46.64; H, 3.58; N, 11.89. Found: C, 46.63; H, 3.63; N, 11.84.

### EXAMPLE 518

This example illustrates the preparation of 2-[2-(2-fluorophenyl)pyrimidin-4-yl]-1,5,6,7-tetrahydro-4H-pyrrolo[3,2-c]pyridin-4-one trifluoroacetate.

The title compound was prepared from 2-fluorobenzonitrile in the same manner as for Example 517. ¹H NMR (400 MHz, DMSO-d₆) δ 12.02 (s, 1H), 8.75 (d, J=5.4 Hz, 1H), 8.18 (m, 1H), 7.75 (d, J=5.5 Hz, 1H), 7.57 (m, 1H), 7.35 (m, 3H), 7.14 (s, 1H), 3.41 (m, 2H), 2.88 (t, J=6.8 Hz, 2H). HRMS calculated for C₁₇H₁₃FN₄O (MH⁺) 309.1146, found 309.1185. Anal. calculated for C₁₇H₁₃FN₄O.0.1 TFA C, 66.94; H, 4.25; N, 18.17. Found: C, 65.94; H, 4.29; N, 18.26.

### EXAMPLE 519

This example illustrates the preparation of 2-[2-(3-fluorophenyl)pyrimidin-4-yl]-1,5,6,7-tetrahydro-4H-pyrrolo[3,2-c]pyridin-4-one trifluoroacetate.

The title compound was prepared from 3-fluorobenzonitrile in the same manner as for Example 517. ¹H NMR (400 MHz, DMSO-d₆) δ 12.15 (s, 1H), 8.74 (d, J=5.5 Hz, 1H), 8.44 (m, 2H), 7.74 (d, J=5.4 Hz, 1H), 7.58 (m, 1H), 7.38 (m, 2H), 7.16 (s, 1H), 3.44 (t, J=6.8 Hz, 2H), 2.94 (t, J=6.8 Hz, 2H). HRMS calculated for C₁₇H₁₃FN₄O (MH⁺) 309.1146, found 309.1117. Anal. calculated for C₁₇H₁₃FN₄O.0.5 TFA0.85 H₂O C, 56.80; H, 4.02; N, 14.72. Found: C, 56.67; H, 3.72; N, 15.04.

### EXAMPLE 520

This example illustrates the preparation of 2-(2-quinolin-3-ylpyrimidin-4-yl)-1,5,6,7-tetrahydro-4H-pyrrolo[3,2-c]pyridin-4-one trifluoroacetate.

The title compound was prepared from quinoline-3-carbonitrile in the same manner as for Example 517. ¹H NMR (400 MHz, DMSO-d₆) δ 12.27 (s, 1H), 10.14 (d, J=2.2 Hz, 1H), 9.62 (d, J=1.6 Hz, 1H), 8.83 (d, J=5.4 Hz, 1H), 8.28 (d, J=7.7 Hz, 1H), 8.18 (d, J=8.6 Hz, 1H), 7.95 (m, 1H), 7.80 (m, 2H), 7.42 (d, J=2.3 Hz, 1H), 7.19 (s, 1H), 3.46 (t, J=6.8 Hz, 2H), 2.97 (t, J=6.8 Hz, 2H). HRMS calculated for C₂₀H₁₅N₅O (MH⁺) 342.1349, found 342.1344. Anal. calculated for C₂₀H₅N₁₅O.2.90 TFA.0.20 H₂O C, 45.87; H, 2.73; N, 10.37. Found: C, 45.88; H, 2.71; N, 10.37.

### EXAMPLE 521

This example illustrates the preparation of 2-pyridin-3-yl-1,5,6,7-tetrahydro-4H-pyrrolo[3,2-c]pyridin-4-one trifluoroacetate.

Ammonium acetate was dried by azeotropic removal of water with ethanol. A mixture of 2-bromo-1-pyridin-3-ylethanone hydrobromide (Aust. J. Chem. 42:1735 (1989)) (6.0 g, 21.4 mmol), piperdiene-2.4-dione (Example 1, step 3) (2.7 g, 23.5 mmol), and anhydrous ammonium acetate (6.6 g, 85.6 mmol) were suspended in ethanol (60 mL) at 0° C. The reaction mixture was allowed to warm to room temperature overnight. The mixture was concentrated, dissolved in methanol/water, and cooled to 4° C. overnight. The precipitate was filtered and washed with cold 10% methanol/water, water, and ether to give crude 2-pyridin-3-yl-1,5,6,7-tetrahydro-4H-pyrrolo[3,2-c]pyridin-4-one as a yellow solid (1.18 g) which was purified by reverse-phase HPLC (acetonitrile/water/0.05% trifluoroacetic acid) to give 2-pyridin-3-yl-1,5,6,7-tetrahydro-4H-pyrrolo[3,2-c]pyridin-4-one trifluoroacetate as an off-white solid (605 mg). The mother liquor was also purified by reverse-phase HPLC (acetonitrile/water/0.05% trifluoroacetic acid) and trituration with methanol/diethyl ether to give 2-pyridin-3-yl-1,5,6,7-tetrahydro-4H-pyrrolo[3,2-c]pyridin-4-one trifluoroacetate as a yellow solid (688 mg). The combined yield of purified 2-pyridin-3-yl-1,5,6,7-tetrahydro-4H-pyrrolo[3,2-c]pyridin-4-one trifluoroacetate was 1.29 g, 3.95 mmol, 18%. ¹H NMR (300 MHz, DMSO-d₆) δ 11.96 (s, 1H), 9.00 (s, 1H), 8.51 (s, 1H), 8.34 (d, J=8.4, 1H), 7.69 (dd, J=8.1, 5.1, 1H), 7.06 (s, 1H), 7.00 (d, J=2.4, 1H), 3.40 (t, J=6.9, 2H), 2.84 (t, J=6.9, 2H). HRMS calculated for C₁₂H₁₂N₂O (MH⁺) 214.0975, found 214.0954.

### EXAMPLE 522

This example illustrates the preparation of 2-[6-(2-fluorophenyl)pyrimidin-4-yl]-1,5,6,7-tetrahydro-4H-pyrrolo[3,2-c]pyridin-4-one hydrochloride.

Step 1. Synthesis of 1-[6-(2-fluorophenyl)pyrimidin-4-yl]ethanone. A mixture of 4-chloro-6-(1-ethoxyvinyl)pyrimidine (Bassani and Lehn, Bull. Soc. Chim. Fr. 1997, 134, 897-906) (1.33 g, 7.20 mmol), 2-fluorophenylboronic acid (1.51 g, 10.8 mmol), tetrakis(triphenylphospine)palladium(0) (416 mg, 0.360 mmol), 2.0 M sodium carbonate (10.8 mL, 21.6 mmol), ethanol (10 mL), and ethylene glycol dimethyl ether (20 mL) was refluxed for 4 hours. The reaction mixture was partitioned between ethyl acetate and water. The organic layer was washed with brine, dried (sodium sulfate), concentrated, and purified by flash chromatography (0→10% ethyl acetate/hexanes) to give 4-(1-ethoxyvinyl)-6-(2-fluorophenyl)pyrimidine as a white solid (762 mg, 3.12 mmol, 43% yield). LC-MS (ES+) MH⁺=245.

4-(1-Ethoxyvinyl)-6-(2-fluorophenyl)pyrimidine (762 mg, 3.12 mmol) was dissolved in acetone (10 mL) and treated with 2 N HCl (5 mL). The solution was stirred at room temperature overnight. The reaction was poured into water and extracted with ethyl acetate. The organic layers were washed with saturated sodium bicarbonate and brine, dried (sodium sulfate), and concentrated to give 1-[6-(2-fluorophenyl)pyrimidin-4-yl]ethanone as an off-white solid (657 mg, 3.04 mmol, 97%). LC-MS (ES+) MH⁺=217. ¹H NMR (400 MHz, CDCl₃) δ 9.40 (d, J=1.4,1H), 8.37 (t, J=1.6, 1H), 8.14 (td, J=7.8, 1.8, 1H), 7.51-7.45 (m, 1H), 7.30 (td, J=7.8, 1.0, 1H), 7.20 (ddd, J=11.5, 8.2, 1.0, 1H), 2.74 (s, 3H).

Step 2. Synthesis of 2-bromo-1-[6-(2-fluorophenyl)pyrimidin-4-yl]ethanone hydrobromide. A solution of 1-[6-(2-fluorophenyl)pyrimidin-4-yl]ethanone (580 mg, 2.68 mmol) and pyridinium tribromide (858 mg, 2.68 mmol) in acetonitrile (5 mL) was treated with 33% hydrogen bromide in acetic acid (0.490 mL, 2.68 mmol). The reaction was stirred at room temperature overnight. The resultant precipitate was filtered and washed with acetonitrile to give the title compound as a pale yellow solid (371 mg, 0.987 mmol, 37% yield). LC-MS (ES+) MH⁺=295, 297.

Step 3. Synthesis of 2-[6-(2-fluorophenyl)pyrimidin-4-yl]-1,5,6,7-tetrahydro-4H-pyrrolo[3,2-c]pyridin-4-one hydrochloride.

Ethanol (4 mL) was added to a mixture of 2-bromo-1-[6-(2-fluorophenyl)pyrimidin-4-yl]ethanone hydrobromide (362 mg, 0.963 mmol), piperdiene-2.4-dione (Example 1, step 3) (120 mg, 1.06 mmol), and ammonium acetate (300 mg, 3.85 mmol). The reaction was stirred at room temperature. A precipitate soon formed. After 2 hours, the precipitate was filtered and washed with water and ether to give a yellow solid (216 mg). The solid was suspended in methanol (10 mL) and treated with 2 M HCl in diethyl ether (0.50 mL). The mixture was diluted with ethyl acetate (10 mL) and stirred for 5 minutes. The precipitate was filtered, washed with ethyl acetate and diethyl ether, and dried under vacuum to give 2-[6-(2-fluorophenyl)pyrimidin-4-yl]-1,5,6,7-tetrahydro-4H-pyrrolo[3,2-c]pyridin-4-one hydrochloride as a yellow solid (225 mg, 0.653 mmol, 68% yield). ¹H NMR (400 MHz, DMSO-d₆) δ 12.33 (s, 1H), 9.14 (d, J=1.2, 1H), 8.13 (s, 1H), 8.00 (td, J=7.8, 1.7, 1H), 7.63-7.56 (m, 1H), 7.44-7.37 (m, 2H), 7.32 (d, J=2.2, 1H), 7.29-7.10 (br s, 1H), 3.40 (t, J=6.8, 2H), 2.86 (t, J=6.8, 2H). HRMS calculated for C₁₇H₁₄FN₄O (MH⁺) 309.1146, found 309.1120.

### EXAMPLE 523

This example illustrates the preparation of 2-[(1E)-3-(3-fluorophenyl)-3-oxoprop-1-enyl]-1,5,6,7-tetrahydro-4H-pyrrolo[3,2-c]pyridin-4-one.

Step 1. (Preparation of 1,5,6,7-tetrahydro-4H-pyrrolo[3,2-c]pyridin-4-one)

To a mixture of 2,4-dioxopiperidine (10 g, 0.088 mol) and aminoacetaldehyde dimethyl acetal (10.5 g, 0.1 mol) in 200 ml of dry benzene was added 0.1 g of p-toluenesulfonic acid monohydrate and the mixture was heated at reflux for 6 h with azeotropic removal of water. The reaction mixture was then evaporated to give a white solid. A solution of the above solid in 125 ml of trifluoroacetic acid was stirred at room temperature for 4 h and then evaporated to dryness under reduced pressure. The oily residue was dissolved in CH2Cl2 and evaporated. The dissolution and evaporation sequence was repeated several times to ensure complete removal of trifluoroacetic acid. The residue was stirred with 400 ml of ether, which resulted in a yellowish precipitate. The mixture was filtered and the precipitate was washed with additional 500 ml of ether. The combined ether filtrates were evaporated to give a yellow solid, which was stirred in 200 ml of hexane, then collected by filtration and air-dried: Yield 7.75 g (76%); ¹HNMR (400 MHz, DMSO-d₆) δ 11.11 (br s, 1H), 6.63 (dd, 1H), 6.18 (dd, 1H), 3.33 (t, 2H), 2.71 (t, 2H); m/z (M+H): 137

Step 2. (Preparation of 2-formyl-1,5,6,7-tetrahydro-4H-pyrrolo[3,2-c]pyridin-4-one).

To a suspension of 1,5,6,7-tetrahydro-4H-pyrrolo[3,2-c]pyridin-4-one (6.8 g, 0.05 mol) from step 1 above in 200 ml of dichloromethane+nitromethane (4:1) cooled at −20° C. was added anhydrous AlCl3 (13.3 g, 0.1 mol) in several portions and the resulting solution was stirred at −20° C. for 10 min. Dichloromethyl methyl ether (11.5 g, 0.1 mol) was added in one portion and the mixture was stored at 0° C. overnight. The reaction mixture was quenched by pouring into 50 ml of ice water and stirred for 30 min. The organic layer was separated and the aqueous layer was further extracted with ethyl acetate (4×100 ml). The organic layers were combined, dried over magnesium sulfate and evaporated to give 3.8 g (46%) of a brown powder, which was used in the next step without further purification: ¹HNMR (400 MHz, DMSO-d₆) δ 9.3 (S, 1H), 8.42 (d, 1H), 7.62 (d,1H), 3.38 (t, 2H), 2.52 (t, 2H); m/z (M+H): 165

Step 3. (Preparation of 2-[(1E)-3-(3-fluorophenyl)-3-oxoprop-1-enyl]-1,5,6,7-tetrahydro-4H-pyrrolo[3,2-c]pyridin-4-one)

To a solution of 2-formyl-1,5,6,7-tetrahydro-4H-pyrrolo[3,2-c]pyridin-4-one (1.64 g, 0.01 mol) and m-fluoroacetophenone (2.76 g, 0.02 mol) in 100 ml of ethanol was added 2 ml of 10% NaOH solution and the mixture was heated at reflux for 10 h. After cooling to room temperature, the mixture was evaporated to dryness and the residue was stirred with 200 ml of ether. The resulting orange precipitate was collected by filtration, washed with ether and air-dried. Purification of the crude product by flash-chromatography on silica gel using 9:1 dichloromethane+methanol eluent gave 1.26 g (44%) of yellow-orange crystalline solid: ¹HNMR (400 MHz, DMSO-d₆) δ 11.05 (br s, 1H), 8.44 (m, 1H), 7.70-7.85 (m, 4H), 7.44 (dd, 1H), 7.13 (s, 1H), 6.75 (d, 1H), 3.47 (t, 2H), 2.46 (t, 2H); m/z (M+H): 285.

### EXAMPLE 524

This example illustrates the preparation of 2-[3-(3-fluorophenyl)-1-methyl-4,5-dihydro-1H-pyrazol-5-yl]-1,5,6,7-tetrahydro-4H-pyrrolo[3,2-c]pyridin-4-one.

A mixture of 2-[(1E)-3-(3-fluorophenyl)-3-oxoprop-1-enyl]-1,5,6,7-tetrahydro-4H-pyrrolo[3,2-c]pyridin-4-one (Example 523) (0.57 g, 2 mmol) and methylhydrazine (0.115 g, 2.5 mmol) in 20 ml of ethanol was warmed at 60° C. for 30 min. After cooling to room temperature, the mixture was evaporated and the residue was stirred with 100 ml of ether. The resulting yellow-orange precipitate was collected by filtration, washed with ether and air-dried. Recrystallization of the crude product from ethanol gave 0.42 g (67%) of yellow crystalline solid: ¹HNMR (400 MHz, DMSO-d₆) δ 10.95 (br s, 1H), 8.56 (m, 1H), 7.82 (d, 1H), 7.35-7.46 (m, 2H), 7.21-7.29 (m, 1H), 7.05 (s, 1H), 4.18 (dd, 1H), 3.72 (dd, 1H), 3.66 (dd, 1H), 3.35 (t, 2H), 2.68 (t, 2H); m/z (M+H): 313.

### EXAMPLE 525

This example illustrates the preparation of (4E)-4-[(3-fluorophenyl)hydrazono]-4-(4-oxo-4,5,6,7-tetrahydro-1H-pyrrolo[3,2-]pyridin-2-yl)butanoic acid.

Step 1. (Preparation of ethyl 4-(4-oxo-4,5,6,7-tetrahydro-1H-pyrrolo[3,2-c]pyridin-2-yl)butanoate).

To a suspension of 1,5,6,7-tetrahydro-4H-pyrrolo[3,2-c]pyridin-4-one (2.72 g, 0.02 mol) in 100 ml of dichloromethane+nitromethane (4:1) cooled at −20° C. was added anhydrous AlCl3 (10.6 g, 0.08 mol) in several portions and the resulting solution was stirred at −20° C. for 10 min. Ethyl succinoyl chloride (6.56 g, 0.04 mol) was added in one portion and the mixture was stored at 0° C. overnight. The reaction mixture was slowly warmed to room temperature, then stirred for additional 1 h and quenched by pouring into 100 ml of ice water. The organic layer was separated and the aqueous layer was further extracted with ethyl acetate (2×100 ml). The organic layers were combined, washed with brine, dried over magnesium sulfate and evaporated. Recrystallization of the residue from ethanol gave 2.24 g (42%) of a pale orange powder: ¹HNMR (400 MHz, DMSO-d₆) δ 12.06 (br s, 1H), 7.20 (br s, 1H), 7.15 (s, 1H), 4.20 (q, 2H), 3.36 (t, 2H), 3.03 (t, 2H), 2.77 (t, 2H), 2.57 (t, 2H), 1.15 (t, 3H); m/z (M+H): 265

Step 2. (Preparation of ethyl (4E)-4-[(3-fluorophenyl)hydrazono]-4-(4-oxo-4,5,6,7-tetrahydro-1H-pyrrolo[3,2-c]pyridin-2-yl)butanoate).

A mixture of the intermediate from step 1 above (2 g, 7.5 mmol), m-fluorophenyl hydrazine hydrochloride (1.3 g, 8 mmol) and diisopropylethylamine (1.3 g, 10 mmol) in 50 ml of ethanol was heated at reflux overnight. After cooling to room temperature, the solution was evaporated and the residue was stirred with 50 ml of water. The resulting precipitate was collected by filtration and recrystallized from ethanol to give 1.85 g (66%) of pale orange solid: ¹H NMR (400 MHz, DMSO-d₆) δ 11.05 (br s, 2H), 7.33 (s, 1H), 7.20 (m, 1H), 7.03-7.10 (m, 1H), 6.82-6.93 (m, 2H), 4.30 (q, 2H), 3.42 (t, 2H), 3.15 (t, 2H), 2.90 (t, 2H), 2.64 (t, 2H), 1.15 (t, 3H); m/z (M+H): 373.

Step 3. (Preparation of (4E)-4-[(3-fluorophenyl)hydrazono]-4-(4-oxo-4,5,6,7-tetrahydro-1H-pyrrolo[3,2-c]pyridin-2-yl)butanoic acid).

To a suspension of the intermediate from step 2 above (1.5 g, 4 mmol) in 100 ml of ethanol was added 10% NaOH (2.5 ml, 6.25 mmol) and the mixture heated at 60° C. for 4 h. After cooling to room temperature, the solution was evaporated, the residue was taken up in 25 ml of water and acidified to pH=1 with dilute HCl. The resulting orange precipitate was collected by filtration, washed with water and air-dried: yield 1.2 g (87%), ¹H NMR (400 MHz, DMSO-d₆) δ 11.20 (br s, 3H), 7.52 (s, 1H), 7.28-7.33 (m, 1H), 6.95-7.10 (m, 1H), 6.79-6.88 (m, 2H), 3.42 (t, 2H), 3.15 (t, 2H), 2.70-2.95 (m, 4H); m/z (M+H): 345.

### EXAMPLE 526

This example illustrates the preparation of 2-[1-(3-fluorophenyl)-6-oxo-1,4,5,6-tetrahydro pyridazin-3-yl]-1,5,6,7-tetrahydro-4H-pyrrolo[3,2-c]pyridin-4-one.

A suspension of (4E)-4-[(3-fluorophenyl)hydrazono]-4-(4-oxo-4,5,6,7-tetrahydro-1H-pyrrolo[3,2-c]pyridin-2-yl)butanoic acid (Example 525) (0.6 g, 1.74 mmol) in 5 ml of acetic acid was heated at reflux for 1 h. After cooling to room temperature, the solution was evaporated to dryness and the residue was stirred with 25 ml of water. The resulting precipitate was collected by filtration, washed with ether and air-dried. Recrystallization of the above solid from ethanol to give 0.39 g (68.5%) of grey solid: ¹HNMR (400 MHz, DMSO-d₆) δ 11.25 (br s, 1H), 7.90 (dd, 1H), 7.62 (m, 1H), 7.48 (m, 1H), 7.36 (s, 1H), 7.11 (m, 1H), 3.46 (m, 2H), 3.15 (t, 2H), 2.86 (m, 2H), 2.57 (t, 2H); m/z (M+H): 327.

### EXAMPLE 527

This example illustrates the preparation of 2-[1-(3-fluorophenyl)-5-oxo-4,5-dihydro-1H-pyrazol-3-yl]-1,5,6,7-tetrahydro-4H-pyrrolo[3,2-c]pyridin-4-one.

Step 1. (Preparation of ethyl 3-(4-oxo-4,5,6,7-tetrahydro-1H-pyrrolo[3,2-c]pyridin-2-yl)propionate).

To a suspension of 1,5,6,7-tetrahydro-4H-pyrrolo[3,2-c]pyridin-4-one (2.04 g, 0.015 mol) in 100 ml of dichloromethane+nitromethane (4:1) cooled at −20° C. was added anhydrous AlCl3 (8 g, 0.06 mol) in several portions and the resulting solution was stirred for 10 min. Ethyl malonyl chloride (4.5 g, 0.03 mol) was added in one portion and the reaction mixture was slowly warmed to room temperature over 1 h. The mixture was stirred for additional 5 h and quenched by pouring into 50 ml of ice water. The organic layer was separated and the aqueous layer was further extracted with ethyl acetate (2×100 ml). The organic layers were combined, washed with brine, dried over magnesium sulfate and evaporated. Recrystallization of the residue from ethanol gave 2.38 g (63%) of an off-white solid: ¹H NMR (400 MHz, DMSO-d₆) δ 12.05 (br s, 1H), 7.18 (br s, 1H), 7.13 (s, 1H), 4.08 (q, 2H), 3.87 (s, 2H), 3.41 (t, 2H), 2.77 (t, 2H), 1.16 (t, 3H); m/z (M+H): 251.

Step 2. (2-[1-(3-fluorophenyl)-5-oxo-4,5-dihydro-1H-pyrazol-3-yl]-1,5,6,7-tetrahydro-4H-pyrrolo[3,2-c]pyridin-4-one).

A mixture of the intermediate from step 1 above (2 g, 7.5 mmol) and m-fluorophenylhydrazine (0.63 g, 5 mmol, freshly prepared from m-fluorophenylhydrazine hydrochloride and sodium methoxide) in 5 ml of acetic acid was heated at reflux for 6 h. After cooling to room temperature, the solution was evaporated and the residue was stirred with 10 ml of water. The resulting precipitate was collected by filtration and recrystallized from ethanol/water to give 0.55 g (44%) of pale brown solid: ¹H NMR (400 MHz, DMSO-d₆) δ 11.05 (br s, 1H), 7.82 (dd, 1H), 7.55 (m, 1H), 7.36 (m, 1H), 7.31 (s, 1H), 7.02 (m, 1H), 3.66 (s, 2H), 3.35 (t, 2H), 2.88 (t, 2H); m/z (M+H): 313.

### EXAMPLE 528

This example illustrates the preparation of 2-(4-hydroxy-3-quinolin-3-ylphenyl)-1,5,6,7-tetrahydro-4H-pyrrolo[3,2-c]pyridin-4-one hydrochloride.

Step 1. (Preparation of 2-(3-bromo-4-methoxyphenyl)-1,5,6,7-tetrahydro-4H-pyrrolo[3,2-c]pyridin-4-one).

To a mixture of 2,3′-dibromo-4′-methoxyacetophenone (3.3 g, 10.7 mmol, prepared as described in Australian J. Chem. 1973, 26, 1327-31), 2,4-dioxopiperidine (1.7 g, 15 mmol) and ammonium acetate (3.3 g, 42.8 mol) in 80 ml of dry ethanol at 0° C. was added 50 ml 2M ammonia in methanol. The mixture was gradually warmed to room temperature over 1 h and then stirred for 18 h. The resulting precipitate was collected by filtration, washed with ether and air-dried to give 850 mg (25%) of pale orange solid: ¹H NMR (400 MHz, DMSO-d₆) δ 11.95 (br s, 1H), 7.88 (d, 1H), 7.53 (s, 1H), 7.22 (s, 1H), 6.80 (d, 1H), 3.74 (s, 3H), 3.40-3.52 (m, 2H), 2.53-2.62 (m, 2H); m/z (M+): 320.

Step 2. (Preparation of 2-(4-methoxy-3-quinolin-3-ylphenyl)-1,5,6,7-tetrahydro-4H-pyrrolo[3,2-c]pyridin-4-one).

A mixture of the intermediate from step 1 above (289 mg, 0.9 mmol), quinoline-3-boronic acid (207.6 mg, 1.2 mmol), Pd(dppf)Cl2 (107.8 mg, 0.132 mmol) and 0.6 ml 2M cesium carbonate in 5 ml of DMF was heated at 80° C. under nitrogen atmosphere for 18 h. After cooling to room temperature, the insoluble material was removed by filtration and the filtrate was concentrated under vacuum. Purification of the residue by reverse phase chromatography (acetonitrile/water) gave 257 mg (77%) of the title compound as a yellow solid: ¹HNMR (400 MHz, DMSO-d₆) δ 11.85 (br s, 1H), 8.90 (s, 1H), 8.33 (br s, 1H), 8.13-8.17 (m, 2H), 7.88 (s, 1H), 7.73-7.80 (m, 2H), 7.43-7.60 (m, 3H), 7.23 (s, 1H), 3.64 (s, 3H), 3.44-3.54 (m, 2H), 2.58-2.66 (m, 2H); m/z (M+H): 370.

Step 3. (Preparation of 2-(4-hydroxy-3-quinolin-3-ylphenyl)-1,5,6,7-tetrahydro-4H-pyrrolo[3,2-c]pyridin-4-one hydrochloride).

To a suspension of the intermediate from step 2 above (147.6 mg, 0.4 mmol) in 10 ml of dichloromethane was added 1 M boron tribromide in dichloromethane (2 ml, 2 mmol) and the solution was stirred at room temperature. After 2 h, the reaction was quenched by the addition of 25 ml of saturated NaHCO₃ solution. The resulting precipitate was collected by filtration, washed with water and dried under vacuum to give an orange solid. To a solution of the above solid in 3 ml of methanol was added 1 ml 2M HCl in ether and stirred for 30 min. Ether (20 ml) was added to the reaction mixture and the resulting precipitate was collected by filtration, washed with ether and dried under vacuum to give 103 mg (66%) of the title compound as an orange solid: ¹HNMR (400 MHz, DMSO-d₆) δ 12.01 (br s, 1H) 8.81 (s, 1H), 8.11-8.17 (m, 2H), 7.70-7.80 (m, 3H), 7.52-7.61 (m, 2H), 7.42 (d, 1H), 7.20 (s, 1H), 3.38-3.46 (m, 2H), 2.56-2.64 (m, 2H); m/z (M+H): 356.

### EXAMPLE 529

Step 1: A 1M solution of trimethylsilylmethyl lithium in pentane (535.00 mL, 535 mmol) was added dropwise to a −78° C. cooled solution of isopropyl pinacolborate (98.90 g, 535 mmol) in THF (650.00 mL) at a rate which allowed the internal reaction temperature to be maintained below −60° C. The resulting colorless solution was maintained at −78° C. overnight, then was warmed to 0° C. and poured into crushed ice with 50 mL of concentrated HCl. After the ice melted, the solution was partitioned between hexanes and water. The hexane phase was dried (MgSO₄), concentrated to a small volume, and then distilled under vacuum to afford 74.34 g of pure pinacol trimethylsilylmethane boronate as a colorless oil (bp 87°-93° C. at 25 torr). ¹H NMR (CDCl₃): δ 1.18 (s, 12H), 0.05 (br s, 2H), 0.00 (s, 9H). GC-MS, m/e 199 (M⁺-Me).

Step 2: 2.5M solution of BuLi in hexanes (4.00 mL, 10.0 mmol) was added dropwise to an ice-water cooled solution of 2,2,6,6-tetramethylpiperidine (1.41 g, 10.0 mmol) and TMEDA (1.16 g, 10.0 mmol) in THF (10.00 mL). After 5 min, a solution of pinacol trimethylsilylmethane. boronate (2.14 g, 10.0 mmol) in THF (5.00 mL) was then added dropwise, and after 30 minutes of reaction at 0° C., a solution of an aldehyde or ketone (10.0 mmol) in THF (5.00 mL) was added dropwise. This was slowly warmed to ambient temperature and allowed to react overnight, then was analyzed by GCMS or LCMS to verify formation of the desired vinylboronate. After partitioning the reaction mixture between EtOAc and dilute agueous HCl, the organic phase was washed with water, dried (MgSO₄), concentrated, and purified on silica gel flash chromatography eluted with a hexane/EtOAc gradient. The purified compounds were characterized by NMR and GCMS. The following compounds were prepared with this method.

| | | Calculated | |
|---|---|---|---|
| Example | | Exact Mass | |
| No. | Compound | (m + H) | Found |
| 530 | 3-[(E/Z)-2-(4,4,5,5-tetramethyl-1,3,2- | 231.10 | 231 (M⁺) |
| | dioxaborolan-2-yl)ethenyl]pyridine | (M⁺) | |
| 531 | 5-[(E/Z)-2-(4,4,5,5-tetramethyl-1,3,2- | 275.1452 | 275.1450 |
| | dioxaborolan-2-yl)ethenyl]-1,3-benzodioxole | | |
| 532 | 3-[(E/Z)-2-(4,4,5,5-tetramethyl-1,3,2- | 282.1663 | 282.1672 |
| | dioxaborolan-2-yl)ethenyl]quinoline | | |
| 533 | 4-[(E/Z)-2-(4,4,5,5-tetramethyl-1,3,2- | 232.1506 | 232.1490 |
| | dioxaborolan-2-yl)ethenyl]pyridine | | |
| 534 | tert-butyl 5-[(E/Z)-2-(4,4,5,5-tetramethyl- | 370.2188 | 370.2192 |
| | 1,3,2-dioxaborolan-2-yl)ethenyl]-1H-indole- | | |
| | 1-carboxylate | | |
| 535 | tert-butyl 3-[(E/Z)-2-(4,4,5,5-tetramethyl- | 463.2557 | 463.2554 |
| | 1,3,2-dioxaborolan-2-yl)ethenyl]-1H-indole- | | |
| | 1-carboxylate | | |
| 536 | 4-[(E/Z)-2-(4,4,5,5-tetramethyl-1,3,2- | 370.2188 | 370.2204 |
| | dioxaborolan-2-yl)ethenyl]-1-trityl-1H-imidazole | | |
| 537 | 3-[(1E,3E)-4-(4,4,5,5-tetramethyl-1,3,2- | 258.1663 | 258.1665 |
| | dioxaborolan-2-yl)buta-1,3-dienyl]pyridine | | |
| 538 | 3-[(1E,3Z)-4-(4,4,5,5-tetramethyl-1,3,2- | 258.1663 | 258 (ES) |
| | dioxaborolan-2-yl)buta-1,3-dienyl]pyridine | 257.14 | 257 (M⁺) |
| | | (M⁺) | |
| 539 | 4,4,5,5-tetramethyl-2-[(1E,3E/1E,3Z)-4- | 256.1638 | 256.1632 |
| | phenylbuta-1,3-dienyl]-1,3,2-dioxaborolane | | |
| 540 | 1-(4-methoxybenzyl)-3-phenyl-4-[(E/Z)-2- | 417.2349 | 417 (ES) |
| | (4,4,5,5-tetramethyl-1,3,2-dioxaborolan-2- | | |
| | yl)vinyl]-1H-pyrazole | | |
| 541 | 4,4,5,5-tetramethyl-2-[(E)-2-thien-3-ylvinyl]- | 236.1042 | 236 (M⁺) |
| | 1,3,2-dioxaborolane | (M⁺) | |
| 542 | 2-[(E)-2-(3-furyl)vinyl]-4,4,5,5- | 220.1271 | 220 (M⁺) |
| | tetramethyl-1,3,2-dioxaborolane | (M⁺) | |
| 543 | 2-[(E)-2-(4-methoxyphenyl)vinyl]-4,4,5,5- | 260.1584 | 260 (M⁺) |
| | tetramethyl-1,3,2-dioxaborolane | (M⁺) | |
| 544 | 4,4,5,5-tetramethyl-2-[(E)-2- | 320.1007 | 320 (M⁺) |
| | (pentafluorophenyl)vinyl]-1,3,2-dioxaborolane | (M⁺) | |
| 545 | 4,4,5,5-tetramethyl-2-[(1E)-2-phenylprop-1- | 244.1635 | 244 (M⁺) |
| | enyl]-1,3,2-dioxaborolane | (M⁺) | |
| 546 | 2-[(E)-2-(2,4-difluorophenyl)vinyl]-4,4,5,5- | 266.1290 | 266 (M⁺) |
| | tetramethyl-1,3,2-dioxaborolane | (M⁺) | |
| 547 | 2-[(E)-2-(2-furyl)vinyl]-4,4,5,5- | 220.1271 | 220 (M⁺) |
| | tetramethyl-1,3,2-dioxaborolane | (M⁺) | |
| 548 | 2-[(E)-2-(2-fluorophenyl)vinyl]-4,4,5,5- | 248.1384 | 248 (M⁺) |
| | tetramethyl-1,3,2-dioxaborolane | (M⁺) | |
| 549 | 2-[(E)-2-(2,5-difluorophenyl)vinyl]-4,4,5,5- | 266.1290 | 266 (M⁺) |
| | tetramethyl-1,3,2-dioxaborolane | (M⁺) | |
| 550 | 4,4,5,5-tetramethyl-2-[(1E)-3-phenylprop-1- | 244.1635 | 244 (M⁺) |
| | enyl]-1,3,2-dioxaborolane | (M⁺) | |

### EXAMPLE 551

A solution of Cs₂CO₃ (0.65 g, 2.0 mmol) in degassed water (100 mL) was added to a mixture of an E/Z-vinylboronate (1.0 mmol), 2-(2-chloropyridin-4-yl)-1,5,6,7-tetrahydro-4H-pyrrolo[3,2-c]pyridin-4-one (0.124 g, 0.5 mmol), and Pd(Ph₃)₄ (30 mg, 26 pmol) in DMF (2.50 mL), and heated overnight at 80° C. under a dry nitrogen atmosphere. The resulting reaction mixture was analyzed by LCMS to verify formation of the desired cross-coupled product, then was purified by reverse-phase C18 chromatography with a water/acetonitrile gradient to separate the E- and Z-isomers. Purified compounds were assayed by analytical reverse phase HPLC, NMR, and MS. The following compounds were prepared with this general method.

| | | Calculated | Found | Found |
|---|---|---|---|---|
| Example | | Exact Mass | Exact Mass | Electrospray |
| No. | Compound | m + H | m + H | m + H |
| 552 | 2-{2-[(E)-2-phenylethenyl]pyridin- | 316.1450 | | 316 |
| | 4-yl}-1,5,6,7-tetrahydro-4H-pyrrolo[3,2- | | | |
| | c]pyridin-4-one | | | |
| 553 | 2-{2-[(E)-1-methyl-2-phenylethenyl]pyridin- | 330.1601 | 330.1562 | |
| | 4-yl}-1,5,6,7-tetrahydro-4H-pyrrolo[3,2- | | | |
| | c]pyridin-4-one trifluoroacetate | | | |
| 554 | 2-{2-[(E)-2-pyridin-3-ylvinyl]pyridin-4- | 317.1397 | 317.1409 | 317 |
| | yl}-1,5,6,7-tetrahydro-4H-pyrrolo[3,2- | | | |
| | c]pyridin-4-one trifluoroacetate | | | |
| 555 | 2-{2-[(E)-2-(1,3-benzodioxol-5- | 360.1343 | 360.1331 | 360 |
| | yl)ethenyl]pyridin-4-yl}-1,5,6,7-tetrahydro- | | | |
| | 4H-pyrrolo[3,2-c]pyridin-4-one trifluoroacetate | | | |
| 556 | 2-{2-[(E)-2-(4-methoxyphenyl)ethenyl]pyridin- | 346.1556 | | 346 |
| | 4-yl}-1,5,6,7-tetrahydro-4H-pyrrolo[3,2- | | | |
| | c]pyridin-4-one | | | |
| 557 | 2-{2-[(E)-2-quinolin-3-ylethenyl]pyridin-4- | 367.1553 | 367.1566 | 367 |
| | yl}-1,5,6,7-tetrahydro-4H-pyrrolo[3,2- | | | |
| | c]pyridin-4-one trifluoroacetate | | | |
| 558 | 2-{2-[(E)-2-pyridin-4-ylethenyl]pyridin-4- | 317.1397 | 317.1381 | 317 |
| | yl}-1,5,6,7-tetrahydro-4H-pyrrolo[3,2- | | | |
| | c]pyridin-4-one trifluoroacetate | | | |
| 559 | 2-(2-{(E)-2-[4- | 384.1318 | 384.1321 | 384 |
| | (trifluoromethyl)phenyl]ethenyl}pyridin-4-yl)- | | | |
| | 1,5,6,7-tetrahydro-4H-pyrrolo[3,2-c]pyridin-4-one | | | |
| 560 | 2-(2-{2-[4-(trifluoromethyl)phenyl]ethyl}pyridin- | 386.1475 | 386.1505 | |
| | 4-yl)-1,5,6,7-tetrahydro-4H-pyrrolo[3,2-c]pyridin-4- | | | |
| | one trifluoroacetate | | | |
| 561 | tert-butyl 5-{(E)-2-[4-(4-oxo-4,5,6,7-tetrahydro-1H- | 455.2078 | 455.2063 | 455 |
| | pyrrolo[3,2-c]pyridin-2-yl)pyridin-2-yl]ethenyl}- | | | |
| | 1H-indole-1-carboxylate trifluoroacetate | | | |
| 562 | 2-{2-[(E)-2-(4-fluorophenyl)ethenyl]pyridin-4- | 334.1356 | | 334 |
| | yl}-1,5,6,7-tetrahydro-4H-pyrrolo[3,2-c]pyridin-4- | | | |
| | one | | | |
| 563 | 2-{2-[(E)-2-(3,4-difluorophenyl)ethenyl]pyridin-4- | 352.1261 | | 352 |
| | yl}-1,5,6,7-tetrahydro-4H-pyrrolo[3,2-c]pyridin-4- | | | |
| | one trifluoroacetate | | | |
| 564 | 2-{2-[(Z)-2-(3,4-difluorophenyl)ethenyl]pyridin-4- | 352.1261 | | 352 |
| | yl}-1,5,6,7-tetrahydro-4H-pyrrolo[3,2-c]pyridin-4- | | | |
| | one trifluoroacetate | | | |
| 565 | 2-{2-[(Z)-2-thien-2-ylethenyl]pyridin-4-yl}- | 322.1014 | | 322 |
| | 1,5,6,7-tetrahydro-4H-pyrrolo[3,2-c]pyridin-4-one | | | |
| 566 | 2-{2-[(E)-2-thien-2-ylethenyl]pyridin-4-yl}- | 322.1014 | | 322 |
| | 1,5,6,7-tetrahydro-4H-pyrrolo[3,2-c]pyridin-4-one | | | |
| 567 | 2-{2-[(Z)-2-(4-methoxyphenyl)ethenyl]pyridin-4- | 346.1556 | | 346 |
| | yl}-1,5,6,7-tetrahydro-4H-pyrrolo[3,2-c]pyridin-4- | | | |
| | one | | | |
| 568 | tert-butyl 3-{(E)-2-[4-(4-oxo-4,5,6,7-tetrahydro-1H- | 455.2078 | 455.2063 | |
| | pyrrolo[3,2-c]pyridin-2-yl)pyridin-2-yl]ethenyl}- | | | |
| | 1H-indole-1-carboxylate trifluoroacetate | | | |
| 569 | 2-{2-[(E)-2-(pentafluorophenyl)ethenyl]pyridin-4- | 406.0979 | | 406 |
| | yl}-1,5,6,7-tetrahydro-4H-pyrrolo[3,2-c]pyridin-4- | | | |
| | one | | | |
| 570 | 2-{2-[(Z)-2-(1-trityl-1H-imidazol-4- | 548.2450 | | 548 (Z) |
| | yl)ethenyl]pyridin-4-yl}-1,5,6,7-tetrahydro-4H- | | | 548 (E) |
| | pyrrolo[3,2-c]pyridin-4-one trifluoroacetate | | | |
| 571 | 2-{2-[(1Z,3E)-4-pyridin-3-ylbuta-1,3-dienyl]pyridin- | 343.1559 | | 343 |
| | 4-yl}-1,5,6,7-tetrahydro-4H-pyrrolo[3,2-c]pyridin- | | | |
| | 4-one trifluoroacetate | | | |
| 572 | 2-{2-[(1E,3E)-4-pyridin-3-ylbuta-1,3-dienyl]pyridin- | 343.1559 | | 343 |
| | 4-yl}-1,5,6,7-tetrahydro-4H-pyrrolo[3,2-c]pyridin- | | | |
| | 4-one trifluoroacetate | | | |
| 573 | 2-{2-[(E)-2-(2,4-difluorophenyl)ethenyl]pyridin-4- | 352.1261 | | 352 |
| | yl}-1,5,6,7-tetrahydro-4H-pyrrolo[3,2-c]pyridin-4- | | | |
| | one | | | |
| 574 | 2-{2-[(1Z)-2-phenylprop-1-enyl]pyridin-4-yl}- | 330.1606 | | 330 |
| | 1,5,6,7-tetrahydro-4H-pyrrolo[3,2-c]pyridin-4-one | | | |
| 575 | 2-{2-[(1E)-2-phenylprop-1-enyl]pyridin-4-yl}- | 330.1606 | | 330 |
| | 1,5,6,7-tetrahydro-4H-pyrrolo[3,2-c]pyridin-4-one | | | |
| 576 | 2-{2-[(Z)-2-pyridin-3-ylethenyl]pyridin-4-yl}- | 317.1397 | 317.1391 | |
| | 1,5,6,7-tetrahydro-4H-pyrrolo[3,2-c]pyridin-4-one | | | |
| | trifluoroacetate | | | |
| 577 | 2-{2-[(1E,3E)-4-phenylbuta-1,3-dienyl]pyridin-4- | 342.1601 | 342.1633 | 342 |
| | yl}-1,5,6,7-tetrahydro-4H-pyrrolo[3,2-c]pyridin- | | | |
| | 4-one trifluoroacetate | | | |
| 578 | 2-{2-[(1Z,3E)-4-phenylbuta-1,3-dienyl]pyridin-4- | 342.1601 | 342.1602 | 342 |
| | yl}-1,5,6,7-tetrahydro-4H-pyrrolo[3,2-c]pyridin-4- | | | |
| | one trifluoroacetate | | | |
| 579 | 2-{2-[(Z)-2-(2-furyl)vinyl]pyridin-4-yl}-1,5,6,7- | 306.1243 | | 306 |
| | tetrahydro-4H-pyrrolo[3,2-c]pyridin-4-one | | | |
| 580 | 2-{2-[(E)-2-(2-furyl)vinyl]pyridin-4-yl}-1,5,6,7- | 306.1243 | | 306 |
| | tetrahydro-4H-pyrrolo[3,2-c]pyridin-4-one | | | |
| 581 | 2-{2-[(1E)-3-(benzyloxy)prop-1-enyl]pyridin-4- | 360.1707 | 360.1699 | |
| | yl}-1,5,6,7-tetrahydro-4H-pyrrolo[3,2-c]pyridin-4- | | | |
| | one trifluoroacetate or 2-{2-[(1E)-3-(benzyloxy)prop- | | | |
| | 1-enyl]pyridin-4-yl}-1,5,6,7-tetrahydro-4H- | | | |
| | pyrrolo[3,2-c]pyridin-4-one trifluoroacetate | | | |
| 582 | 2-{2-[3-(benzyloxy)propyl]pyridin-4-yl}-1,5,6,7- | 362.1863 | 362.1868 | |
| | tetrahydro-4H-pyrrolo[3,2-c]pyridin-4-one | | | |
| | trifluoroacetate or 2-{2-[3- | | | |
| | (benzyloxy)propyl]pyridin-4-yl}-1,5,6,7-tetrahydro- | | | |
| | 4H-pyrrolo[3,2-c]pyridin-4-one trifluoroacetate | | | |
| 583 | 2-(2-{(E)-2-[1-(4-methoxybenzyl)-3-phenyl-1H-pyrazol- | 502.2238 | 502.2251 | |
| | 4-yl]vinyl}pyridin-4-yl)-1,5,6,7-tetrahydro-4H- | | | |
| | pyrrolo[3,2-c]pyridin-4-one | | | |
| 584 | 2-{2-[(Z)-2-thien-3-ylvinyl]pyridin-4-yl}- | 322.1014 | | 322 |
| | 1,5,6,7-tetrahydro-4H-pyrrolo[3,2-c]pyridin-4-one | | | |
| 585 | 2-{2-[(E)-2-thien-3-ylvinyl]pyridin-4-yl}- | 322.1014 | | 322 |
| | 1,5,6,7-tetrahydro-4H-pyrrolo[3,2-c]pyridin-4-one | | | |
| 586 | 2-{2-[(Z)-2-(3-furyl)vinyl]pyridin-4-yl}-1,5,6,7- | 306.1243 | | 306 |
| | tetrahydro-4H-pyrrolo[3,2-c]pyridin-4-one | | | |
| 587 | 2-{2-[(E)-2-(3-furyl)vinyl]pyridin-4-yl}-1,5,6,7- | 3,06.1243 | | 306 |
| | tetrahydro-4H-pyrrolo[3,2-c]pyridin-4-one | | | |
| 588 | 2-{2-[(E)-2-(3-phenyl-1H-pyrazol-4-yl)vinyl]pyridin- | 382.1662 | 382.1652 | |
| | 4-yl}-1,5,6,7-tetrahydro-4H-pyrrolo[3,2-c]pyridin- | | | |
| | 4-one trifluoroacetate | | | |
| 589 | 2-{2-[(Z)-2-(3-phenyl-1H-pyrazol-4-yl)vinyl]pyridin- | 382.1662 | 382.1666 | |
| | 4-yl}-1,5,6,7-tetrahydro-4H-pyrrolo[3,2-c]pyridin- | | | |
| | 4-one trifluoroacetate | | | |
| 590 | 2-{2-[(E)-2-(2-fluorophenyl)vinyl]pyridin-4-yl}- | 334.1356 | | 334 |
| | 1,5,6,7-tetrahydro-4H-pyrrolo[3,2-c]pyridin-4-one | | | |
| 591 | 2-{2-[(E)-2-(2,5-difluorophenyl)vinyl]pyridin-4- | 352.1261 | | 352 |
| | yl}-1,5,6,7-tetrahydro-4H-pyrrolo[3,2-c]pyridin-4- | | | |
| | one | | | |
| 592 | 2-(2-{(E)-2-[2-fluoro-4- | | | 402 |
| | (trifluoromethyl)phenyl]vinyl}pyridin-4-yl)-1,5,6,7- | | | |
| | tetrahydro-4H-pyrrolo[3,2-c]pyridin-4-one | | | |
| | trifluoroacetate | | | |
| 593 | 2-{2-[(E)-2-(2-methylphenyl)vinyl]pyridin-4-yl}- | | | 330 |
| | 1,5,6,7-tetrahydro-4H-pyrrolo[3,2-c]pyridin-4-one | | | |
| | trifluoroacetate | | | |
| 594 | 2-{2-[(E)-2-(3-chlorophenyl)vinyl]pyridin-4-yl}- | | | 350 |
| | 1,5,6,7-tetrahydro-4H-pyrrolo[3,2-c]pyridin-4-one | | | |
| | trifluoroacetate | | | |
| 595 | 2-{2-[(E)-2-(2-fluoro-4-methoxyphenyl)vinyl]pyridin- | | | 364 |
| | 4-yl}-1,5,6,7-tetrahydro-4H-pyrrolo[3,2-c]pyridin- | | | |
| | 4-one trifluoroacetate | | | |
| 596 | 2-{2-[(E)-2-(3,4-dimethoxyphenyl)vinyl]pyridin-4- | | | 376 |
| | yl}-1,5,6,7-tetrahydro-4H-pyrrolo[3,2-c]pyridin-4- | | | |
| | one | | | |
| 597 | 2-{2-[(E)-2-(2-naphthyl)vinyl]pyridin-4-yl}- | | | 366 |
| | 1,5,6,7-tetrahydro-4H-pyrrolo[3,2-c]pyridin-4-one | | | |
| | trifluoroacetate | | | |
| 598 | 2-{2-[(E)-2-(2-fluoro-5-methoxyphenyl)vinyl]pyridin- | | | 364 |
| | 4-yl}-1,5,6,7-tetrahydro-4H-pyrrolo[3,2-c]pyridin- | | | |
| | 4-one trifluoroacetate | | | |
| 599 | 2-{(Z)-2-[4-(4-oxo-4,5,6,7-tetrahydro-1H- | | | 341 |
| | pyrrolo[3,2-c]pyridin-2-yl)pyridin-2- | | | |
| | yl]vinyl}benzonitrile trifluoroacetate | | | |
| 600 | 2-{2-[(E)-2-(2,3-dimethoxyphenyl)vinyl]pyridin-4- | | | 376 |
| | yl}-1,5,6,7-tetrahydro-4H-pyrrolo[3,2-c]pyridin-4- | | | |
| | one trifluoroacetate | | | |
| 601 | 2-{2-[(E)-2-(4-chloro-2-fluorophenyl)vinyl]pyridin- | | | 368 |
| | 4-yl}-1,5,6,7-tetrahydro-4H-pyrrolo[3,2-c]pyridin- | | | |
| | 4-one trifluoroacetate | | | |
| 602 | 2-{2-[(E)-2-(2,3-dihydro-1H-inden-5- | | | 356 |
| | yl)vinyl]pyridin-4-yl}-1,5,6,7-tetrahydro-4H- | | | |
| | pyrrolo[3,2-c]pyridin-4-one trifluoroacetate | | | |
| 603 | 2-{2-[(Z)-2-(2-chlorophenyl)vinyl]pyridin-4-yl}- | | | 350 |
| | 1,5,6,7-tetrahydro-4H-pyrrolo[3,2-c]pyridin-4-one | | | |
| 604 | 2-{2-[(E)-2-(2-chlorophenyl)vinyl]pyridin-4-yl}- | | | 350 |
| | 1,5,6,7-tetrahydro-4H-pyrrolo[3,2-c]pyridin-4-one | | | |
| | trifluoroacetate | | | |
| 605 | 2-{2-[(E)-2-(3,4-dichlorophenyl)vinyl]pyridin-4- | | | 384 |
| | yl}-1,5,6,7-tetrahydro-4H-pyrrolo[3,2-c]pyridin-4- | | | |
| | one trifluoroacetate | | | |
| 606 | 2-{2-[(E)-2-(6-methoxy-2-naphthyl)vinyl]pyridin-4- | | | 396 |
| | yl}-1,5,6,7-tetrahydro-4H-pyrrolo[3,2-c]pyridin-4- | | | |
| | one trifluoroacetate | | | |
| 607 | 2-{2-[(E)-2-(1-naphthyl)vinyl]pyridin-4-yl}- | | | 366 |
| | 1,5,6,7-tetrahydro-4H-pyrrolo[3,2-c]pyridin-4-one | | | |
| | trifluoroacetate | | | |
| 608 | 2-(2-{(Z)-2-[3,5- | | | 452 |
| | bis(trifluoromethyl)phenyl]vinyl}pyridin-4-yl)-1,5,6,7- | | | |
| | tetrahydro-4H-pyrrolo[3,2-c]pyridin-4-one | | | |
| 609 | 2-(2-{(E)-2-[3,5- | | | 452 |
| | bis(trifluoromethyl)phenyl]vinyl}pyridin-4-yl)-1,5,6,7- | | | |
| | tetrahydro-4H-pyrrolo[3,2-c]pyridin-4-one | | | |
| | trifluoroacetate | | | |
| 610 | 2-{2-[(Z)-2-(2,3-dihydro-1,4-benzodioxin-6- | | | 374 |
| | yl)vinyl]pyridin-4-yl}-1,5,6,7-tetrahydro-4H- | | | |
| | pyrrolo[3,2-c]pyridin-4-one | | | |
| 611 | 2-{2-[(E)-2-(2,3-dihydro-1,4-benzodioxin-6- | | | 374 |
| | yl)vinyl]pyridin-4-yl}-1,5,6,7-tetrahydro-4H- | | | |
| | pyrrolo[3,2-c]pyridin-4-one | | | |
| 612 | 2-(2-{(E)-2-[3- | | | 384 |
| | (trifluoromethyl)phenyl]vinyl}pyridin-4-yl)-1,5,6,7- | | | |
| | tetrahydro-4H-pyrrolo[3,2-c]pyridin-4-one | | | |
| | trifluoroacetate | | | |
| 613 | 2-{2-[(E)-2-(2,4-dichlorophenyl)vinyl]pyridin-4- | | | 384 |
| | yl}-1,5,6,7-tetrahydro-4H-pyrrolo[3,2-c]pyridin-4- | | | |
| | one trifluoroacetate | | | |
| 614 | 2-{2-[(Z)-2-(2,4-dichlorophenyl)vinyl]pyridin-4- | | | 384 |
| | yl}-1,5,6,7-tetrahydro-4H-pyrrolo[3,2-c]pyridin-4- | | | |
| | one | | | |
| 615 | 2-{2-[(E)-2-(4-methylphenyl)vinyl]pyridin-4-yl}- | | | 330 |
| | 1,5,6,7-tetrahydro-4H-pyrrolo[3,2-c]pyridin-4-one | | | |
| | trifluoroacetate | | | |
| 616 | 2-{2-[(Z)-2-(3-fluoro-2-methylphenyl)vinyl]pyridin- | | | 348 |
| | 4-yl}-1,5,6,7-tetrahydro-4H-pyrrolo[3,2-c]pyridin- | | | |
| | 4-one | | | |
| 617 | 2-{2-[(E)-2-(3-fluoro-2-methylphenyl)vinyl]pyridin- | | | 348 |
| | 4-yl}-1,5,6,7-tetrahydro-4H-pyrrolo[3,2-c]pyridin- | | | |
| | 4-one trifluoroacetate | | | |
| 618 | 2-{2-[(E)-2-(4-methoxy-3-methylphenyl)vinyl]pyridin- | | | 360 |
| | 4-yl}-1,5,6,7-tetrahydro-4H-pyrrolo[3,2-c]pyridin- | | | |
| | 4-one trifluoroacetate | | | |
| 619 | 2-(2-{(E)-2-[4-(benzyloxy)-3- | | | 452 |
| | methoxyphenyl]vinyl}pyridin-4-yl)-1,5,6,7-tetrahydro- | | | |
| | 4H-pyrrolo[3,2-c]pyridin-4-one trifluoroacetate | | | |
| 620 | 2-{2-[(E)-2-(4-chloro-3-fluorophenyl)vinyl]pyridin- | | | 368 |
| | 4-yl}-1,5,6,7-tetrahydro-4H-pyrrolo[3,2-c]pyridin-4- | | | |
| | one trifluoroacetate | | | |
| 621 | 2-{2-[(E)-2-(3,5-dimethoxyphenyl)vinyl]pyridin-4- | | | 376 |
| | yl}-1,5,6,7-tetrahydro-4H-pyrrolo[3,2-c]pyridin-4- | | | |
| | one trifluoroacetate | | | |
| 622 | 2-{2-[(E)-2-(3,4,5-trimethoxyphenyl)vinyl]pyridin-4- | | | 406 |
| | yl}-1,5,6,7-tetrahydro-4H-pyrrolo[3,2-c]pyridin-4- | | | |
| | one trifluoroacetate | | | |
| 623 | 2-{2-[(E)-2-(3-phenoxyphenyl)vinyl]pyridin-4-yl}- | | | 408 |
| | 1,5,6,7-tetrahydro-4H-pyrrolo[3,2-c]pyridin-4-one | | | |
| | trifluoroacetate | | | |
| 624 | 2-{2-[(E)-2-(1,1′-biphenyl-4-yl)vinyl]pyridin-4- | | | 392 |
| | yl}-1,5,6,7-tetrahydro-4H-pyrrolo[3,2-c]pyridin-4- | | | |
| | one trifluoroacetate | | | |
| 625 | 2-{2-[(E)-2-(1,3-benzodioxol-4-yl)vinyl]pyridin-4- | | | 360 |
| | yl}-1,5,6,7-tetrahydro-4H-pyrrolo[3,2-c]pyridin-4- | | | |
| | one trifluoroacetate | | | |
| 626 | 2-(2-{(E)-2-[2- | | | 384 |
| | (trifluoromethyl)phenyl]vinyl}pyridin-4-yl)-1,5,6,7- | | | |
| | tetrahydro-4H-pyrrolo[3,2-c]pyridin-4-one | | | |
| | trifluoroacetate | | | |
| 627 | 2-{2-[(E)-2-(2-methoxyphenyl)vinyl]pyridin-4-yl}- | | | 346 |
| | 1,5,6,7-tetrahydro-4H-pyrrolo[3,2-c]pyridin-4-one | | | |
| | trifluoroacetate | | | |
| 628 | 2-{2-[(E)-2-(6-methoxypyridin-3-yl)vinyl]pyridin-4- | 346.143 | 347.15 | |
| | yl}-1,5,6,7-tetrahydro-4H-pyrrolo[3,2-c]pyridin-4- | | | |
| | one | | | |
| 629 | 2-{2-[(E)-2-(6-phenoxypyridin-3-yl)vinyl]pyridin-4- | 408.159 | 409.18 | |
| | yl}-1,5,6,7-tetrahydro-4H-pyrrolo[3,2-c]pyridin-4-one | | | |
| 630 | 2-{2-[(E)-2-(1-benzothien-2-yl)vinyl]pyridin-4- | 371.109 | 372.12 | |
| | yl}-1,5,6,7-tetrahydro-4H-pyrrolo[3,2-c]pyridin-4-one | | | |
| 631 | 2-{2-[(E)-2-(1-benzofuran-2-yl)vinyl]pyridin-4- | 355.132 | 356.1 | |
| | yl}-1,5,6,7-tetrahydro-4H-pyrrolo[3,2-c]pyridin-4-one | | | |
| 632 | 2-{2-[(Z)-2-(1-benzofuran-2-yl)vinyl]pyridin-4- | 355.132 | 356.1 | |
| | yl}-1,5,6,7-tetrahydro-4H-pyrrolo[3,2-c]pyridin-4-one | | | |
| 633 | 2-{2-[(Z)-2-(1-benzothien-2-yl)vinyl]pyridin-4- | 371.109 | 372.12 | |
| | yl}-1,5,6,7-tetrahydro-4H-pyrrolo[3,2-c]pyridin-4-one | | | |
| 634 | N,N-diethyl-4-{(E)-2-[4-(4-oxo-4,5,6,7-tetrahydro-1H- | 414.2056 | 414.9 | |
| | pyrrolo[3,2-c]pyridin-2-yl)pyridin-2- | | | |
| | yl]vinyl}benzamide trifluoroacetate | | | |
| 635 | 2-{2-[(E)-2-(4-{[(2R)-2-(pyrrolidin-1-ylmethyl)pyrrolidin- | 495.6298 | 496 | |
| | 1-yl]carbonyl}phenyl)vinyl]pyridin- | | | |
| | 4-yl}-1,5,6,7-tetrahydro-4H-pyrrolo[3,2-c]pyridin- | | | |
| | 4-one trifluoroacetate | | | |
| 636 | 2-(2-{(E)-2-[5-(1,3-dioxolan-2-yl)-2- | 377.1376 | 378.2 | |
| | furyl]vinyl}pyridin-4-yl)-1,5,6,7-tetrahydro-4H- | | | |
| | pyrrolo[3,2-c]pyridin-4-one | | | |
| 637 | 2-(2-{(Z)-2-[5-(1,3-dioxolan-2-yl)-2- | 377.1376 | 378.2 | |
| | furyl]vinyl}pyridin-4-yl)-1,5,6,7-tetrahydro-4H- | | | |
| | pyrrolo[3,2-c]pyridin-4-one | | | |
| 638 | 2-{2-[(E)-2-(3-methylthien-2-yl)vinyl]pyridin-4- | 335.1092 | 336.1 | |
| | yl}-1,5,6,7-tetrahydro-4H-pyrrolo[3,2-c]pyridin-4-one | | | |
| 639 | N-[2-(dimethylamino)ethyl]-N-methyl-4-{(E)-2-[4- | 443.2321 | 444 | |
| | (4-oxo-4,5,6,7-tetrahydro-1H-pyrrolo[3,2-c]pyridin-2- | | | |
| | yl)pyridin-2-yl]vinyl}benzamide trifluoroacetate | | | |
| 640 | 2-{2-[(Z)-2-(4-{[(2R)-2-(pyrrolidin-1- | 495.2634 | 496 | |
| | ylmethyl)pyrrolidin-1-yl]carbonyl}phenyl)vinyl]pyridin- | | | |
| | 4-yl}-1,5,6,7-tetrahydro-4H-pyrrolo[3,2-c]pyridin-4-one | | | |
| | trifluoroacetate | | | |
| 641 | 2-{2-[(Z)-2-(3-methylthien-2-yl)vinyl]pyridin-4-yl}- | 335.1092 | 336.1 | |
| | 1,5,6,7-tetrahydro-4H-pyrrolo[3,2-c]pyridin-4-one | | | |
| 642 | 2-{2-[(E)-2-(4-chloro-1-methyl-1H-pyrazol-3- | 353.1043 | 354.2 | |
| | yl)vinyl]pyridin-4-yl}-1,5,6,7-tetrahydro-4H- | | | |
| | pyrrolo[3,2-c]pyridin-4-one | | | |
| 643 | 2-{2-[(E)-2-(5-phenyi-2-furyl)vinyl]pyridin-4-yl}- | 381.1477 | 382.2 | |
| | 1,5,6,7-tetrahydro-4H-pyrrolo[3,2-c]pyridin-4-one | | | |
| 644 | 2-{2-[(E)-2-(5-phenylthien-2-yl)vinyl]pyridin-4-yl}- | 397.1249 | 398.1 | |
| | 1,5,6,7-tetrahydro-4H-pyrrolo[3,2-c]pyridin-4-one | | | |
| 645 | 2-(2-{(E)-2-[5-(4-chlorophenyl)-2- | 415.1088 | 416.1 | |
| | furyl]vinyl}pyridin-4-yl)-1,5,6,7-tetrahydro-4H- | | | |
| | pyrrolo[3,2-c]pyridin-4-one | | | |
| 646 | 2-(2-{(E)-2-[5-(3-chlorophenyl)-2- | 415.1088 | 416.1 | |
| | furyl]vinyl}pyridin-4-yl)-1,5,6,7-tetrahydro-4H- | | | |
| | pyrrolo[3,2-c]pyridin-4-one | | | |
| 647 | 2-(2-{(E)-2-[5-(2-chlorophenyl)-2- | 415.1088 | 416.1 | |
| | furyl]vinyl}pyridin-4-yl)-1,5,6,7-tetrahydro-4H- | | | |
| | pyrrolo[3,2-c]pyridin-4-one | | | |
| 648 | 2-{2-[(1Z)-3-phenylprop-1-enyl]pyridin-4-yl}- | 330.1606 | | 330 |
| | 1,5,6,7-tetrahydro-4H-pyrrolo[3,2-c]pyridin-4-one | | | |
| 649 | 2-{2-[(1E)-3-phenylprop-1-enyl]pyridin-4-yl}- | 330.1606 | | 330 |
| | 1,5,6,7-tetrahydro-4H-pyrrolo[3,2-c]pyridin-4-one | | | |

### EXAMPLE 650

This example illustrates the preparation of 2-{2-[(E)-2-(4,5-dimethyl-2-furyl)vinyl]pyridin-4-yl}-1,5,6,7-tetrahydro-4H-pyrrolo[3,2-c]pyridin-4-one.

¹H NMR (400 MHz, CD₃OD) δ 8.34 (d, J=5.6 Hz, 1H), 7.68 (s, 1H), 7.36 (dd, J=5.4 Hz, JJ=2.0 Hz, 1H), 7.27 (d, J=16.0 Hz, 1H), 7.06 (s, 1H), 6.91 (d, J=16.0 Hz, 1H), 6.34 (s, 1H), 3.57 (t, J=7.2 Hz, 2H), 2.94 (t, J=7.2 Hz, 2H), 2.25 (s, 3H), 1.95 (s, 3H); ESI-MS m/z 334 [M+H]⁺.

### EXAMPLE 651

This example illustrates the preparation of 2-{2-[(Z)-2-(4,5-dimethyl-2-furyl)vinyl]pyridin-4-yl}-1,5,6,7-tetrahydro-4H-pyrrolo[3,2-c]pyridin-4-one.

¹H NMR (400 MHz, CD₃OD) δ 8.40 (d, J=5.6 Hz, 1H), 7.90 (s, 1H), 7.42 (dd, J=5.6 Hz, JJ=1.6 Hz, 1H), 6.99 (s, 1H), 6.45 (d, J=12.8 Hz, 1H), 6.38 (s, 1H), 6.31 (d, J=12.8 Hz, 1H), 3.56 (t, J=7.2 Hz, 2H), 2.92 (t, J=7.2 Hz, 2H), 2.07 (s, 3H), 1.89 (s, 3H); ESI-MS m/z 334 [M+H]⁺.

### EXAMPLE 652

This example illustrates the preparation of 2-[2-((Z)-2-{5-[3-(trifluoromethyl)phenyl]-2-furyl}vinyl)pyridin-4-yl]-1,5,6,7-tetrahydro-4H-pyrrolo[3,2-c]pyridin-4-one.

¹H NMR (400 MHz, DMSO) δ 11.87 (s, 1H), 8.55 (d, J=5.2 Hz, 1H), 7.78-7.82 (m, 3H), 7.52-7.56 (m, 2H), 7.44 (t, J=8.0 Hz, 1H), 7.31 (d, J=3.6 Hz, 1H), 7.23 (d, J=3.6 Hz, 1H), 7.00 (s, 1H), 6.95 (d, J=2.0 Hz, 1H), 6.64 (d, J=12.8 Hz, 1H), 6.54 (d, J=12.8 Hz, 1H), 3.33-3.37 (m, 2H), 2.75 (t, J=6.8 Hz, 2H); ESI-MS m/z 450 [M+H]⁺.

### EXAMPLE 653

This example illustrates the preparation of 2-[2-((Z)-2-{5-[3-(trifluoromethoxy)phenyl]-2-furyl}vinyl)pyridin-4-yl]-1,5,6,7-tetrahydro-4H-pyrrolo[3,2-c]pyridin-4-one.

¹H NMR (400 MHz, CD₃OD) δ 8.47 (d, J=5.6 Hz, 1H), 7.95 (s, 1H), 7.55 (dd, J=5.6 Hz, JJ=1.6 Hz, 1H), 7.35 (d, J=8.0 Hz, 1H), 7.16-7.20 (m, 2H), 7.03-7.04 (m, 2H), 6.90 (d, J=3.6 Hz, 1H), 6.70 (d, J=3.2 Hz, 1H), 6.64 (d, J=12.4 Hz, 1H), 6.55 (d, J=12.8 Hz, 1H), 3.51 (t, J=7.2 Hz, 2H), 2.82 (t, J=7.2 Hz, 2H); ESI-MS m/z 466 [M+H]⁺.

### EXAMPLE 654

This example illustrates the preparation of 2-[2-((E)-2-{5-[3-(trifluoromethoxy)phenyl]-2-furyl}vinyl)pyridin-4-yl]-1,5,6,7-tetrahydro-4H-pyrrolo[3,2-c]pyridin-4-one.

¹H NMR (400 MHz, CD₃OD) δ 8.40 (d, J=5.6 Hz, 1H), 7.80 (s, 1H), 7.76 (d, J=7.6 Hz, 1H), 7.64 (s, 1H), 7.51 (t, J=8.0 Hz, 1H), 7.45 (d, J=16 Hz, 1H), 7.41 (dd, J=5.6 Hz, JJ=1.6 Hz, 1H), 7.18-7.22 (m, 2H), 7.09 (s, 1H), 6.98 (d, J=3.6 Hz, 1H), 6.70 (d, J=3.6 Hz, 1H), 3.58 (t, J=7.2 Hz, 2H), 2.95 (t, J=7.2 Hz, 2H); ESI-MS m/z 466 [M+H]⁺.

### EXAMPLE 655

This example illustrates the preparation of 2-{2-[(Z)-2-(2-phenyl-1,3-thiazol-4-yl)vinyl]pyridin-4-yl}-1,5,6,7-tetrahydro-4H-pyrrolo[3,2-c]pyridin-4-one.

¹H NMR (400 MHz, CD₃OD) δ 8.44 (d, J=5.6 Hz, 1H), 7.98 (s, 1H), 7.69 (d, J=7.6 Hz, 2H), 7.62 (s, 1H), 7.50 (dd, J=5.6 Hz, JJ=1.6 Hz, 1H), 7.34 (t, J=7.6 Hz, 1H), 7.25 (t, J=7.6 Hz, 2H), 6.99 (s, 1H), 6.88 (d, J=12.4 Hz, 1H), 6.73 (d, J=12.8 Hz, 1H), 3.50 (t, J=7.2 Hz, 2H), 2.80 (t, J=7.2 Hz, 2H); ESI-MS m/z 399 [M+H]⁺.

### EXAMPLE 656

This example illustrates the preparation of 2-{2-[(E)-2-(2-phenyl-1,3-thiazol-4-yl)vinyl]pyridin-4-yl}-1,5,6,7-tetrahydro-4H-pyrrolo[3,2-c]pyridin-4-one.

¹H NMR (400 MHz, CD₃OD) δ 8.42 (d, J=5.2 Hz, 1H), 8.01-8.04 (m, 2H), 7.82 (s, 1H), 7.62 (s, 2H), 7.60 (s, 1H), 7.48-7.50 (m, 3H), 7.44 (dd, J=5.6 Hz, JJ=2.0 Hz, 1H), 7.10 (s, 1H), 3.58 (t, J=7.2 Hz, 2H), 2.96 (t, J=7.2 Hz, 2H); ESI-MS m/z 399 [M+H]⁺.

### EXAMPLE 657

This example illustrates the preparation of 2-{2-[(E)-2-(2,4-dimethyl-1,3-thiazol-5-yl)vinyl]pyridin-4-yl}-1,5,6,7-tetrahydro-4H-pyrrolo[3,2-c]pyridin-4-one.

¹H NMR (400 MHz, CD₃OD) δ 8.40 (d, J=5.2 Hz, 1H), 7.75 (d, J=15.6 Hz, 1H), 7.73 (s, 1H), 7.41 (dd, J=5.6 Hz, JJ=1.6 Hz, 1H), 7.09 (s, 1H), 6.78 (d, J=16 Hz, 1H), 3.58 (t, J=7.2 Hz, 2H), 2.95 (t, J=7.2 Hz, 2H), 2.65 (s, 3H), 2.47 (s, 3H); ESI-MS m/z 351 [M+H]⁺.

### EXAMPLE 658

This example illustrates the preparation of 2-{2-[(Z)-2-(2,4-dimethyl-1,3-thiazol-5-yl)vinyl]pyridin-4-yl}-1,5,6,7-tetrahydro-4H-pyrrolo[3,2-c]pyridin-4-one.

¹H NMR (400 MHz, CD₃OD) δ 8.52 (d, J=5.6 Hz, 1H), 7.58 (s, 1H), 7.50 (dd, J=5.6 Hz, JJ=1.6 Hz, 1H), 7.04 (s, 1H), 6.90 (d, J=12.4 Hz, 1H), 6.60 (d, J=12.8 Hz, 1H), 3.57 (t, J=6.8 Hz, 2H), 2.92-2.96 (m, 2H), 2.55 (s, 3H), 2.42 (s, 3H); ESI-MS m/z 351 [M+H]⁺.

### EXAMPLE 659

This example illustrates the preparation of 2-{(E)-2-[4-(4-oxo-4,5,6,7-tetrahydro-1H-pyrrolo[3,2-c]pyridin-2-yl)pyridin-2-yl]vinyl}benzonitrile trifluoroacetate.

mp 195-199° C.; ¹H NMR (300 MHz, DMSO-d₆) δ 12.59 (s, 1H), 8.64 (d, J=5.9 Hz, 1H), 8.32 (s, 1H), 8.11-7.81 (m, 5H), 7.63-7.54 (m, 2H), 7.35 (s, 1H), 7.21 (s, 1H), 3.46-337 (m, 2H), 2.92 (t, J=6.7 Hz, 2H); ESI-MS m/z 341 [M+H]⁺.

### EXAMPLE 660

This example illustrates the preparation of 2-{2-[(E)-2-(2,5-dimethylphenyl)vinyl]pyridin-4-yl}-1,5,6,7-tetrahydro-4H-pyrrolo[3,2-c]pyridin-4-one trifluoroacetate.

mp 206-209° C.; ¹H NMR (300 MHz, DMSO-d₆) δ 12.36 (s, 1H), 8.61 (d, J=6.2 Hz, 1H), 8.30 (s, 1H), 8.07 (d, J=16.2 Hz, 1H), 7.81 (d, J=5.9 Hz, 1H), 7.52 (s, 2H), 7.25-7.13 (m, 4H), 3.46-3.42 (m, 2H), 2.93 (t, J=6.7 Hz, 2H), 2.44 (s, 3H), 2.33 (s, 3H); ESI-MS m/z 344 [M+H]⁺.

### EXAMPLE 661

This example illustrates the preparation of 2-(2-{(E)-2-[2-(trifluoromethoxy)phenyl]vinyl}pyridin-4-yl)-1,5,6,7-tetrahydro-4H-pyrrolo[3,2-c]pyridin-4-one trifluoroacetate.

mp 163-167° C.; ¹H NMR (300 MHz, DMSO-d₆) δ 12.39 (s, 1H), 8.62 (d, J=5.9 Hz, 1H), 8.30 (s, 1H), 8.03 (d, J=16.3 Hz, 1H), 7.9 (dd, J=6.9, 1.8 Hz, 1H), 7.83 (d, J=5.0 Hz, 1H), 7.59-7.37 (m, 5H), 7.23 (s, 1H), 3.46-3.39 (m, 2H), 2.92 (t, J=6.7 Hz, 2H); ESI-MS m/z 400 [M+H]⁺.

### EXAMPLE 662

This example illustrates the preparation of 2-(2-{(E)-2-[2-(methylthio)phenyl]vinyl}pyridin-4-yl)-1,5,6,7-tetrahydro-4H-pyrrolo[3,2-c]pyridin-4-one trifluoroacetate.

mp 213-216° C.; ¹H NMR (300 MHz, DMSO-d₆) δ 12.35 (s, 1H), 8.60 (d, J=6.0 Hz, 1H), 8.23 (s, 1H) 8.12 (d, J=16.1 Hz, 1H), 7.81 (d, J=4.9 Hz, 1H), 7.70 (d, J=7.7 Hz, 1H), 7.44-7.42 (m, 3H), 7.31-7.21 (m, 3H), 3.46-3.37 (m, 2H), 2.92 (t, J=6.7 Hz, 2H), 2.55 (s, 3H); ESI-MS m/z 362 [M+H]⁺.

### EXAMPLE 663

This example illustrates the preparation of 2-{2-[(E)-2-(2-morpholin-4-ylphenyl)vinyl]pyridin-4-yl}-1,5,6,7-tetrahydro-4H-pyrrolo[3,2-c]pyridin-4-one trifluoroacetate.

¹H NMR (300 MHz, DMSO-d₆) δ 12.54 (br s, 1H), 8.61 (d, J=6.4 Hz, 1H), 8.43 (s, 1H), 8.09 (d, J=16.4 Hz, 1H), 7.93 (d, J=5.8 Hz, 1H), 7.65 (d, J=7.6 Hz, 1H), 7.60 (s, 1H), 7.56-7.20 (m, 5H), 3.95-3.80 (m, 4H), 3.05-3.89 (m, 6H); ESI-MS m/z 401 [M+H]⁺.

### EXAMPLE 664

This example illustrates the preparation of 2-{2-[(Z)-2-pyrimidin-5-ylvinyl]pyridin-4-yl}-1,5,6,7-tetrahydro-4H-pyrrolo[3,2-c]pyridin-4-one.

¹H NMR (400 MHz, CD₃OD) δ 8.94 (s,l1H), 8.70 (s, 2H), 8.35 (d, J=5.6 Hz, 1H), 7.52 (s, 1H), 7.46 (dd, J=5.6 Hz, JJ=2.0 Hz, 1H), 6.98 (s, 1H), 6.95 (d, J=12.4 Hz, 1H), 6.79 (d, J=12.4 Hz, 1H), 3.55 (t, J=7.2 Hz, 2H), 2.91 (t, J=7.2 Hz, 2H); ESI-MS m/z 318 [M+H]⁺.

### EXAMPLE 665

This example illustrates the preparation of 2-{2-[(E)-2-(2,3-dimethylphenyl)vinyl]pyridin-4-yl}-1,5,6,7-tetrahydro-4H-pyrrolo[3,2-c]pyridin-4-one trifluoroacetate.

mp 217-221° C.; ¹H NMR (300 MHz, DMSO-d₆) δ 12.45 (s, 1H), 8.61 (d, J=6.2 Hz, 1H), 8.36 (s, 1H), 8.24 (d, J=16.2 Hz, 1H), 7.84 (d, J=5.7 Hz, 1H), 7.53-7.51 (m, 2H), 7.26-7.18 (m, 3H), 7.11 (d, J=16.1 Hz, 1H), 3.47-3.42 (m, 2H), 2.93 (t, J=6.8 Hz, 2H), 2.39 (s, 3H), 2.31 (s, 3H); ESI-MS m/z 344 [M+H]⁺.

### EXAMPLE 666

This example illustrates the preparation of 2-{2-[(E)-2-(2-ethylphenyl)vinyl]pyridin-4-yl}-1,5,6,7-tetrahydro-4H-pyrrolo[3,2-c]pyridin-4-one trifluoroacetate.

mp 180-184° C.; ¹H NMR (300 MHz, DMSO-d₆) δ 12.50 (s, 1H), 8.62 (d, J=6.2 Hz, 1H), 8.35 (s, 1H), 8.18 (d, J=16.2 Hz, 1H), 7.85 (d, J=6.1 Hz, 1H), 7.71 (d, J=7.3 Hz, 1H), 7.52 (s, 1H), 7.38-7.29 (m, 3H), 7.26 (s, 1H), 7.20 (d, J=16.2 Hz, 1H), 3.47-3.42 (m, 2H), 2.95-2.87 (m, 4H), 1.20 (t, J=7.5 Hz, 3H); ESI-MS m/z 344 [M+H]⁺.

### EXAMPLE 667

This example illustrates the preparation of 2-{2-[(E)-2-(2,5-dichlorophenyl)vinyl]pyridin-4-yl}-1,5,6,7-tetrahydro-4H-pyrrolo[3,2-c]pyridin-4-one trifluoroacetate.

mp 179-183° C.; ¹H NMR (300 MHz, DMSO-d₆) δ 12.26 (s, 1H), 8.61 (d, J=5.8 Hz, 1H), 8.1 (s, 1H), 8.03-7.98 (m, 2H), 7.75 (d, J=4.7 Hz, 1H), 7.61 (d, J=8.6 Hz, 1H), 7.51-7.47 (m 1H), 7.43 (d, J=16.1 Hz, 1H), 7.33 (s, 1H), 7.20 (s, 1H), 3.45-3.42 (m, 2H), 2.92 (t, J=6.8 Hz, 2H); ESI-MS m/z 384 [M+H]⁺.

### EXAMPLE 668

This example illustrates the preparation of 2-{2-[(E)-2-(2-chloro-6-fluorophenyl)vinyl]pyridin-4-yl}-1,5,6,7-tetrahydro-4H-pyrrolo[3,2-c]pyridin-4-one trifluoroacetate.

mp 235-240° C.; ¹H NMR (300 MHz, DMSO-d₆) δ 12.25 (s, 1H), 8.61 (d, J=5.8 Hz, 1H), 8.20 (s, 1H), 7.91 (d, J=16.5 Hz, 1H), 7.78 (d, J=6.1 Hz, 1H), 7.52-7.30 (m, 5H), 7.20 (s, 1H), 3.48-3.40 (m, 2H), 2.90 (t, J=6.7 Hz, 2H); ESI-MS m/z 368 [M+H]⁺.

### EXAMPLE 669

This example illustrates the preparation of 2-{2-[(E)-2-(2,5-dimethoxyphenyl)vinyl]pyridin-4-yl}-1,5,6,7-tetrahydro-4H-pyrrolo[3,2-c]pyridin-4-one trifluoroacetate.

mp 205-209° C.; ¹H NMR (300 MHz, DMSO-d₆) δ 2.49 (s, 1H), 8.58 (d, J=6.2 Hz, 1H), 8.35 (s, 1H), 8.06 (d, J=16.5 Hz, 1H), 7.86 (d, J=6.5 Hz, 1H), 7.53 (s, 1H), 7.42 (d, J=16.5 Hz, 1H), 7.26-7.21 (m, 2H), 7.10-7.00 (m, 2H), 3.88 (s, 3H), 3.78 (s, 3H), 3.44 (t, J=6.7 Hz, 2H), 2.92 (t, J=6.6 Hz, 2H); ESI-MS m/z 376 [M+H]⁺.

### EXAMPLE 670

This example illustrates the preparation of 2-(2-{(E)-2-[4-(dimethylamino)phenyl]vinyl}pyridin-4-yl)-1,5,6,7-tetrahydro-4H-pyrrolo[3,2-c]pyridin-4-one trifluoroacetate.

mp 230-236° C.; ¹H NMR (300 MHz, DMSO-d₆) δ 12.42 (br s, 1H), 8.51 (d, J=6.6 Hz, 1H), 8.42 (s, 1H), 7.93 (d, J=16.0 Hz, 1H), 7.82 (d, J=6.6 Hz, 1H), 7.62 (s, 1H), 7.53 (d, J=6.8 Hz, 2H), 7.29 (s, 1H), 7.03 (d, J=16.0 Hz, 1H), 6.82 (d, J=6.8 Hz, 2H), 3.52-3.39 (m, 2H), 3.02 (s, 6H), 3.01-2.86 (m, 2H); ESI-MS m/z 359 [M+H]⁺.

### EXAMPLE 671

This example illustrates the preparation of 2-{2-[(E)-2-(1,1′-biphenyl-2-yl)vinyl]pyridin-4-yl}-1,5,6,7-tetrahydro-4H-pyrrolo[3,2-c]pyridin-4-one trifluoroacetate.

mp 154-159° C.; ¹H NMR (300 MHz, DMSO-d₆) δ 12.28 (s, 1H), 8.51 (d, J=5.9 Hz, 1H), 8.09 (s, 1H), 7.86 (dd, J=5.6, 3.5 Hz, 1H), 7.75 (d, J=16.0 Hz, 1H), 7.75 (d, J=6.6 Hz, 1H), 7.56-7.32 (m, 9H), 7.23 (s, 1H), 7.22-7.16 (m, 1H), 3.40-3.39 (m, 2H), 2.88 (t, J=6.7 Hz, 2H); ESI-MS m/z 392 [M+H]⁺.

### EXAMPLE 672

This example illustrates the preparation of 2-(2-{(E)-2-[2-(4-methylpiperazin-1-yl)phenyl]vinyl}pyridin-4-yl)-1,5,6,7-tetrahydro-4H-pyrrolo[3,2-c]pyridin-4-one trifluoroacetate.

mp 132-136° C.; ¹H NMR (300 MHz, DMSO-d₆) δ 12.32 (s, 1H), 9.74 (s, 1H), 8.60 (d, J=5.8 Hz, 1H), 8.18 (s, 1H), 7.99 (d, J=16.4 Hz, 1H), 7.75 (s, 1H), 7.70 (d, J=7.6 Hz, 1H), 7.45-7.14 (m, 6H), 3.57-3.32 (m, 8H), 3.08-2.89 (m, 6H); ESI-MS m/z 414 [M+H]⁺.

### EXAMPLE 673

This example illustrates the preparation of 2-{2-[(E)-2-(2,6-difluorophenyl)vinyl]pyridin-4-yl}-1,5,6,7-tetrahydro-4H-pyrrolo[3,2-c]pyridin-4-one trifluoroacetate.

mp 225-228° C.; ¹H NMR (300 MHz, DMSO-d₆) δ 12.40 (s, 1H), 8.60 (d, J=5.9 Hz, 1H), 8.32 (s, 1H), 7.88 (d, J=16.8 Hz, 1H), 7.82 (s, 1H), 7.65-7.50 (m, 3H), 7.29-7.22 (m, 3H), 3.46-3.42 (m, 2H), 2.91 (t, J=6.7 Hz, 2H), ESI-MS m/z 352 [M+H]⁺.

### EXAMPLE 674

This example illustrates the preparation of 2-(2-{(E)-2-[3-(1H-pyrrol-1-yl)phenyl]vinyl}pyridin-4-yl)-1,5,6,7-tetrahydro-4H-pyrrolo[3,2-c]pyridin-4-one trifluoroacetate.

mp 178-181° C.; ¹H NMR (300 MHz, DMSO-d₆) δ 12.47 (s, 1H), 8.62 (d, J=6.1 Hz, 1H), 8.35 (s, 1H), 7.98 (d, J=16.5 Hz, 1H), 7.90-7.80 (m, 2H), 7.66-7.43 (m, 7H), 7.25 (s, 1H), 6.32 (t, J=2.1 Hz, 2H), 3.46-3.42 (m, 2H), 2.92 (t, J=6.7 Hz, 2H); ESI-MS m/z 381 [M+H]⁺.

### EXAMPLE 675

This example illustrates the preparation of 2-{2-[(E)-2-(3,5-dimethylphenyl)vinyl]pyridin-4-yl}-1,5,6,7-tetrahydro-4H-pyrrolo[3,2-c]pyridin-4-one trifluoroacetate.

mp 205-209° C.; ¹H NMR (300 MHz, DMSO-d₆) δ 12.40 (br s, 1H), 8.60 (d, J=6.1 Hz, 1H), 8.36 (br s, 1H), 7.89 (d, J=16.5 Hz, 1H), 7.83 (d, J=6.2 Hz, 1H), 7.51 (br s, 1H), 7.28-7.22 (m, 4H), 7.09 (s, 1H), 3.44 (t, J=6.9 Hz, 2H), 2.92 (t, J=6.7 Hz, 2H), 2.34 (s, 6H); ESI-MS m/z 344 [M+H]⁺.

### EXAMPLE 676

This example illustrates the preparation of 2-{2-[(Z)-2-(3,5-dimethylphenyl)vinyl]pyridin-4-yl}-1,5,6,7-tetrahydro-4H-pyrrolo[3,2-c]pyridin-4-one.

mp 133-137° C.; ¹H NMR (300 MHz, DMSO-d₆) δ 11.90 (br s, 1H), 8.45 (d, J=5.3 Hz, 1H), 7.49-7.44 (m, 2H), 7.06 (br s, 1H), 6.96 (s, 2H), 6.89 (s, 1H), 6.75-6.69 (m, 2H), 6.60 (d, J=12.6 Hz, 1H), 3.40-3.34 (m, 2H), 2.80 (t, J=6.8 Hz, 2H), 2.17 (s, 6H); ESI-MS m/z 344 [M+H]⁺.

### EXAMPLE 677

This example illustrates the preparation of 2-{2-[(E)-2-(2,6-dimethylphenyl)vinyl]pyridin-4-yl}-1,5,6,7-tetrahydro-4H-pyrrolo[3,2-c]pyridin-4-one trifluoroacetate.

mp 174-179° C.; ¹H NMR (300 MHz, DMSO-d₆) δ 12.33 (s, 1H), 8.62 (d, J=6.0 Hz, 1H), 8.31 (s, 1H), 7.96 (d, J=16.5 Hz, 1H), 7.87-7.79 (m, 1H), 7.52 (s, 1H), 7.25 (s, 1H), 7.19-7.08 (m, 3H), 6.80 (d, J=16.5 Hz, 1H), 3.47-3.43 (m, 2H), 2.92 (t, J=6.8 Hz, 2H), 2.39 (s, 6H); ESI-MS m/z 344 [M+H]⁺.

This preparation also yielded the following two by-products:

### EXAMPLE 678

This example illustrates the preparation of 2-{2-[(Z)-2-(2,6-dimethylphenyl)vinyl]pyridin-4-yl}-1,5,6,7-tetrahydro-4H-pyrrolo[3,2-c]pyridin-4-one trifluoroacetate.

mp 230-235° C.; ¹H NMR (300 MHz, DMSO-d₆) δ 12.33 (s, 1H), 8.64 (d, J=6.2 Hz, 1H), 7.73 (d, J=6.1 Hz, 1H), 7.33-7.12 (m, 5H), 7.02 (s, 1H), 6.85 (d, J=12.2 Hz, 1H), 6.35 (d, J=1.8 Hz, 1H), 3.41-3.31 (m, 2H), 2.83 (t, J=6.7 Hz, 2H), 2.13 (s, 6H); ESI-MS m/z 344 [M+H]⁺.

### EXAMPLE 679

This example illustrates the preparation of 2-{2-[2-(2,6-dimethylphenyl)ethyl]pyridin-⁴-yl}-1,5,6,7-tetrahydro-4H-pyrrolo[3,2-c]pyridin-4-one trifluoroacetate.

mp 158-163° C.; ¹H NMR (300 MHz, DMSO-d₆) δ 12.47 (s, 1H), 8.64 (d, J=6.4 Hz, 1H), 8.04 (s, 1H), 7.96 (d, J=5.8 Hz, 1H), 7.55 (s, 1H), 7.26 (s, 1H), 7.02 (s, 3H), 3.47-3.41 (m, 2H), 3.10-2.8 (m, 6H), 2.34 (s, 6H); ESI-MS m/z 346 [M+H]⁺.

### EXAMPLE 680

This example illustrates the preparation of 2-{2-[(E)-2-(2-bromophenyl)vinyl]pyridin-4-yl}-1,5,6,7-tetrahydro-4H-pyrrolo[3,2-c]pyridin-4-one trifluoroacetate.

mp 201-206° C.; ¹H NMR (300 MHz, DMSO-d₆) δ 12.22.(s, 1H), 8.57 (d, J=5.5 Hz, 1H), 8.05-8.00 (m, 2H), 7.88 (d, J=6.5 Hz, 1H), 7.74-7.71 (m, 2H), 7.49 (t, J=7.4 Hz, 1H), 7.35-7.15 (m, 4H), 3.45-3.39 (m, 2H), 2.89 (t, J=6.7 Hz, 2H); ESI-MS m/z 394 [M+H]⁺.

### EXAMPLE 681

This example illustrates the preparation of 2-{2-[(E)-2-(2,4,5-trimethylphenyl)vinyl]pyridin-4-yl}-1,5,6,7-tetrahydro-4H-pyrrolo[3,2-c]pyridin-4-one trifluoroacetate.

mp 225-229° C.; ¹H NMR (300 MHz, DMSO-d₆) δ 12.40 (br s, 1H), 8.61 (d, J=6.3 Hz, 1H), 8.33 (br s, 1H), 8.05 (d, J=16.4 Hz, 1H), 7.83 (d, J=5.9 Hz, 1H), 7.55 (br s, 1H), 7.49 (s, 1H), 7.28 (br s, 1H), 7.14 (d, J=16.3 Hz, 1H), 7.08 (s, 1H), 3.44 (t, J=6.7 Hz, 2H), 2.93 (t, J=6.6 Hz, 2H), 2.43 (s, 3H), 2.25 (s, 3H), 2.23 (s, 3H); ESI-MS m/z 358 [M+H]⁺.

### EXAMPLE 682

This example illustrates the preparation of 2-{2-[(Z)-2-(2,4,5-trimethylphenyl)vinyl]pyridin-4-yl}-1,5,6,7-tetrahydro-4H-pyrrolo[3,2-c]pyridin-4-one.

mp 133-135° C.; ¹H NMR (300 MHz, DMSO-d₆) δ 11.80 (br s, 1H), 8.40 (d, J=5.2 Hz, 1H), 7.34 (dd, J=5.3, 1.6 Hz, 1H), 7.12 (s, 1H), 7.02-7.01 (m, 2H), 6.90-6.87 (m, 2H), 6.64 (d, J=12.4 Hz, 1H), 6.40 (s, 1H), 3.39-3.33 (m, 2H), 2.78 (t, J=6.8 Hz, 2H), 2.22 (s, 3H), 2.14 (s, 3H); 2.05 (s, 3H); ESI-MS m/z 358 [M+H]⁺.

### EXAMPLE 683

This example illustrates the preparation of 2-{2-[(E)-2-(2-piperidin-1-ylphenyl)vinyl]pyridin-4-yl}-1,5,6,7-tetrahydro-4H-pyrrolo[3,2-c]pyridin-4-one trifluoroacetate.

mp 143-147° C.; ¹H NMR (300 MHz, DMSO-d₆) δ 12.50 (br s, 1H), 8.60 (d, J=6.3 Hz, 1H), 8.40 (br s, 1H), 8.04 (d, J=16.4 Hz, 1H), 7.89 (d, J=5.3 Hz, 1H), 7.64-7.59 (m, 2H), 7.30-7.27 (m, 1H), 7.24 (d, J=16.4 Hz, 1H), 7.17-7.11 (m, 3H), 3.45 (t, J=6.8 Hz, 2H), 2.96-2.91 (m, 6H), 1.73 (br s, 4H), 1.55 (br s, 2H); ESI-MS m/z 399 [M+H]⁺.

### EXAMPLE 684

This example illustrates the preparation of 2-{2-[(Z)-2-(2-piperidin-1-ylphenyl)vinyl]pyridin-4-yl}-1,5,6,7-tetrahydro-4H-pyrrolo[3,2-c]pyridin-4-one.

mp 148-152° C.; ¹H NMR (300 MHz, DMSO-d₆) δ 11.80 (br s, 1H), 8.41 (d, J=5.1 Hz, 1H), 7.40-7.37 (m, 2H), 7.21-7.00 (m, 4H), 6.82-6.78 (m, 2H), 6.61 (d, J=12.5 Hz, 1H), 6.52 (d, J=2.3 Hz, 1H), 3.39-3.33 (m, 2H), 2.95 (br s, 4H), 2.80 (t, J=6.7 Hz, 2H), 1.62 (br s, 4H), 1.53 (br s, 2H); ESI-MS m/z 399 [M+H]⁺.

### EXAMPLE 685

This example illustrates the preparation of 2-{2-[(E)-2-(2-phenoxyphenyl)vinyl]pyridin-4-yl}-1,5,6,7-tetrahydro-4H-pyrrolo[3,2-c]pyridin-4-one trifluoroacetate.

mp 165-170° C.; ¹H NMR (300 MHz, DMSO-d₆) δ 12.38 (s, 1H), 8.56 (d, J=6.1 Hz, 1H), 8.28 (s, 1H), 8.09 (d, J=16.4 Hz, 1H), 7.88-6.78 (m, 2H), 7.53-7.37 (m, 5H), 7.32-7.13 (m, 3H), 7.09 (d, J=7.8 Hz, 2H), 6.91 (d, J=8.1 Hz, 1H), 3.47-3.40 (m, 2H), 2.91 (t, J=6.8 Hz, 2H); ESI-MS m/z 408 [M+H]⁺.

### EXAMPLE 686

This example illustrates the preparation of 2-(2-{(E)-2-[2-(dipropylamino)phenyl]vinyl}pyridin-4-yl)-1,5,6,7-tetrahydro-4H-pyrrolo[3,2-c]pyridin-4-one trifluoroacetate.

mp 160-163° C.; ¹H NMR (300 MHz, DMSO-d₆) δ 12.73 (s, 1H), 8.58 (d, J=6.2 Hz, 1H), 8.39 (s, 1H), 8.16 (d, J=15.4 Hz, 1H), 7.91 (d, J=5.3 Hz, 1H), 7.66 (d, J=7.7 Hz, 1H), 7.50 (s, 1H), 7.39-7.15 (m, 5H), 3.46-3.42 (m, 2H), 2.98-3.10 (m, 4H), 2.93 (t, J=6.7 Hz, 2H), 1.47-1.40 (m, 4H), 0.80 (t, J=7.3 Hz, 6H); ESI-MS m/z 415 [M+H]⁺.

### EXAMPLE 687

This example illustrates the preparation of 2-{2-[(E)-2-(2,6-dichlorophenyl)vinyl]pyridin-4-yl}-1,5,6,7-tetrahydro-4H-pyrrolo[3,2-c]pyridin-4-one trifluoroacetate.

mp 158-163° C.; ¹H NMR (300 MHz, DMSO-d₆) δ 12.33 (s, 1H), 8.62 (d, J=5.9 Hz, 1H), 8.29 (s, 1H), 7.88, (d, J=16.5 Hz, 1H), 7.83 (d, J=5.8 Hz, 1H), 7.61 (d, J=8.0 Hz, 2H), 7.47-7.38 (m, 2H), 7.25 (d, J=16.5 Hz, 1H), 7.20 (s, 1H), 3.45-3.40 (m, 2H), 2.91 (t, J=6.7 Hz, 2H); ESI-MS m/z 384 [M+H]⁺.

### EXAMPLE 688

This example illustrates the preparation of 2-(2-{(E)-2-[2-(phenylthio)phenyl]vinyl)pyridin-4-yl)-1,5,6,7-tetrahydro-4H-pyrrolo[3,2-c]pyridin-4-one trifluoroacetate.

mp 127-132° C.; ¹H NMR (300 MHz, DMSO-d₆) δ 12.32 (s, 1H), 8.57 (d, J=6.0 Hz, 1H), 8.25 (d, J=16.0 Hz, 1H), 8.16 (s, 1H), 7.86 (d, J=7.5 Hz, 1H), 7.78 (d, J=5.8 Hz, 1H), 7.52-7.16 (m, 11H), 3.46-3.41 (m, 2H), 2.91 (t, J=6.7 Hz, 2H); ESI-MS m/z 424 [M+H]⁺.

### EXAMPLE 689

This example illustrates the preparation of 2-{2-[(E)-2-(2-pyrrolidin-1-ylphenyl)vinyl]pyridin-4-yl}-1,5,6,7-tetrahydro-4H-pyrrolo[3,2-c]pyridin-4-one trifluoroacetate.

mp 150-153° C.; ¹H NMR (300 MHz, DMSO-d₆) δ 12.39 (br s, 1H), 8.56 (d, J=6.4 Hz, 1H), 8.42 (br s, 1H), 8.14 (d, J=16.1 Hz, 1H), 8.00-7.90 (m, 1H), 7.61 (br s, 1H), 7.50 (d, J=7.2 Hz, 1H), 7.28-7.26 (m, 2H), 7.00-6.91 (m, 3H), 3.44 (t, J=6.8 Hz, 2H), 3.30 (br s, 4H), 2.93 (t, J=6.8 Hz, 2H), 1.91 (br s, 4H); ESI-MS m/z 385 [M+H]⁺.

### EXAMPLE 690

This example illustrates the preparation of 2-(2-{(E)-2-[4-(morpholin-4-ylcarbonyl)phenyl]vinyl}pyridin-4-yl)-1,5,6,7-tetrahydro-4H-pyrrolo[3,2-c]pyridin-4-one trifluoroacetate.

mp 204-208° C.; ¹H NMR (300 MHz, DMSO-d₆) δ 12.32 (s, 1H), 8.61 (d. J=6.0 Hz, 1H), 8.29 (s, 1H), 7.93 (d, J=16.1 Hz, 1H), 7.79 (d, J=4.7 Hz, 1H), 7.74 (d, J=8.2 Hz, 2H), 7.53 (d, J=8.2 Hz, 2H), 7.46 (s, 1H), 7.34 (d, J=16.4 Hz, 1H), 7.24 (s, 1H), 3.61-3.42 (m, 10H), 2.92 (t, J=6.6 Hz, 2H); ESI-MS m/z 429 [M+H]⁺.

### EXAMPLE 691

This example illustrates the preparation of 2-(2-{(E)-2-[2-(dimethylamino)phenyl]vinyl}pyridin-4-yl)-1,5,6,7-tetrahydro-4H-pyrrolo[3,2-c]pyridin-4-one trifluoroacetate.

mp 220-225° C.; ¹H NMR (300 MHz, DMSO-d₆) δ 12.37 (br s, 1H), 8.57 (d, J=6.2 Hz, 1H), 8.32 (br s, 1H), 8.06 (d, J=16.4 Hz, 1H), 7.81 (br s, 1H), 7.63 (d, J=7.1 Hz, 1H), 7.50 (br s, 1H), 7.41-7.35 (m, 1H), 7.25-7.07 (m, 4H), 3.44 (t, J=6.4 Hz, 2H), 2.93 (t, J=6.7 Hz, 2H), 2.75 (s, 6H); ESI-MS m/z 359 [M+H]⁺.

### EXAMPLE 692

This example illustrates the preparation of 2-(2-{(E)-2-[2-(benzyloxy)phenyl]vinyl}pyridin-4-yl)-1,5,6,7-tetrahydro-4H-pyrrolo[3,2-c]pyridin-4-one trifluoroacetate.

mp 214-218° C.; ¹H NMR (300 MHz, DMSO-d₆) δ 12.43 (s, 1H), 8.59 (d, J=6.0 Hz, 1H), 8.29 (s, 1H), 8.12 (d, J=16.5 Hz, 1H), 7.84 (d, J=5.3 Hz, 1H), 7.68 (d, J=6.5 Hz, 1H), 7.53-7.51 (m, 3H), 7.43-7.30 (m, 5H), 7.26 (s, 1H), 7.19 (d, J=8.3 Hz, 1H), 7.06 (t, J=7.4 Hz, 1H), 5.34 (s, 2H), 3.45-3.43 (m, 2H), 2.93 (t, J=6.4 Hz, 2H); ESI-MS m/z 422 [M+H]⁺.

### EXAMPLE 693

This example illustrates the preparation of 2-(2-{(E)-2-[2,4-bis(dimethylamino)phenyl]vinyl}pyridin-4-yl)-1,5,6,7-tetrahydro-4H-pyrrolo[3,2-c]pyridin-4-one trifluoroacetate.

mp 155-158° C.; ¹H NMR (300 MHz, CD₃OD) δ 8.31 (d, J=6.7 Hz, 1H), 8.23 (s, 1H), 8.12 (d, J=16.1 Hz, 1H), 7.74 (d, J=6.6 Hz, 1H), 7.65 (d, J=8.8 Hz, 1H), 7.47 (s, 1H), 7.05 (d, J=16.0 Hz, 1H), 6.65 (d, J=8.8 Hz, 1H), 6.57 (s, 1H), 3.62 (t, J=6.9 Hz, 2H), 3.10 (s, 6H), 3.03 (t, J=6.9 Hz, 2H), 2.94 (s, 3H); m/z 402 [M+H]⁺.

### EXAMPLE 694

This example illustrates the preparation of 2-{2-[(E)-2-(2-isopropylphenyl)vinyl]pyridin-4-yl}-1,5,6,7-tetrahydro-4H-pyrrolo[3,2-c]pyridin-4-one trifluoroacetate.

mp 220-224° C.; ¹H NMR (300 MHz, DMSO-d₆) δ 12.63 (s, 1H), 8.61 (d, J=6.2 Hz, 1H), 8.42-8.30 (m, 2H), 7.88 (d, J=5.7 Hz, 1H), 7.66 (d, J=7.7 Hz, 1H), 7.53 (s, 1H), 7.43-7.41 (m, 2H), 7.34-7.26 (m, 2H), 7.17 (d, J=16.1 Hz, 1H), 3.55-3.52 (m, 1H), 3.47-3.42 (m, 2H), 2.93 (t, J=6.8 Hz, 2H), 1.25 (d, J=6.8 Hz, 6H); ESI-MS m/z 358 [M+H]⁺.

### EXAMPLE 695

This example illustrates the preparation of 2-{2-[(E)-2-(2-cyclohexylphenyl)vinyl]pyridin-4-yl}-1,5,6,7-tetrahydro-4H-pyrrolo[3,2-c]pyridin-4-one trifluoroacetate.

mp 168-170° C.; ¹H NMR (300 MHz, DMSO-d₆) δ 12.35 (br s, 1H), 8.63 (d, J=6.0 Hz, 1H), 8.29 (br s, 1H), 8.22 (d, J=16.0 Hz, 1H), 7.81 (d, J=5.7 Hz, 1H), 7.64 (d, J=7.6 Hz, 1H), 7.51 (br s, 1H), 7.40-7.27 (m, 4H), 7.09 (d, J=16.0 Hz, 1H), 3.44 (t, J=6.7 Hz, 2H), 3.04 (br s, 1H), 2.93 (t, J=6.6 Hz, 2H), 1.78 (br s, 5H), 1.50-1.44 (m, 4H), 1.28 (br s, 1H); ESI-MS m/z 398 [M+H]⁺.

### EXAMPLE 696

This example illustrates the preparation of 2-(2-{(E)-2-[4-(morpholin-4-ylmethyl)phenyl]vinyl}pyridin-4-yl)-1,5,6,7-tetrahydro-4H-pyrrolo[3,2-c]pyridin-4-one trifluoroacetate.

mp 213-216° C.; ¹H NMR (300 MHz, DMSO-d₆) δ 12.39 (br s, 1H), 10.20 (br s, 1H), 8.62 (d, J=6.0 Hz, 1H), 8.30 (br s, 1H), 7.94 (d, J=16.2 Hz, 1H), 7.79-7.76 (m, 3H), 7.60 (d, J=8.1 Hz, 2H), 7.45 (s, 1H), 7.37 (d, J=16.3 Hz, 1H), 7.25 (s, 1H), 4.39 (br s, 2H), 4.10-3.90 (m, 2H), 3.80-3.60 (m, 2H), 3.44 (t, J=6.6 Hz, 2H), 3.40-3.10 (m, 4H), 2.92 (t, J=6.8Hz, 2H); ESI-MS m/z 415 [M+H]⁺.

### EXAMPLE 697

This example illustrates the preparation of 2-{2-[(E)-2-(4-piperidin-1-ylphenyl)vinyl]pyridin-4-yl}-1,5,6,7-tetrahydro-4H-pyrrolo[3,2-c]pyridin-4-one trifluoroacetate.

mp 171-175° C.; ¹H NMR (300 MHz, DMSO-d₆) δ 12.50 (s, 1H), 8.54 (d, J=6.5 Hz, 1H), 8.42 (s, 1H), 7.91 (d, J=16.2 Hz, 1H), 7.83 (dd, J=5.0, 1.5 Hz, 1H), 7.63 (d, J=2.3 Hz, 1H), 7.52 (d, J=8.8 Hz, 2H), 7.31 (s, 1H), 7.06-6.99 (m, 3H), 3.50-3.42 (m, 2H), 3.40-3.30 (m, 4H), 2.94 (t, J=6.7 Hz, 2H), 1.70-1.50 (m, 6H); ESI-MS m/z 399 [M+H]⁺.

### EXAMPLE 698

This example illustrates the preparation of 2-{2-[(E)-2-(2-fluoro-4-morpholin-4-ylphenyl)vinyl]pyridin-4-yl}-1,5,6,7-tetrahydro-4H-pyrrolo[3,2-c]pyridin-4-one trifluoroacetate.

¹H NMR (300 MHz, DMSO-d₆) δ 12.40 (br s, 1H), 8.55 (d, J=6.3 Hz, 1H), 8.34 (s, 1H), 7.91 (d, J=16.6 Hz, 1H), 7.81 (d, J=6.0 Hz, 1H), 7.66-7.49 (m, 2H), 7.28 (s, 1H), 7.13 (d, J=16.6 Hz, 1H), 7.00-6.80 (m, 2H), 3.80-3.69 (m, 4H), 3.37-3.20 (m, 4H), 3.00-2.88 (m, 2H); m/z 419 [M+H]⁺; Anal. Calculated for C₂₄H₂₃FN₄O₂-1.125CF₃CO₂OH—H₂O: C, 55.69; H, 4.59; N, 9.92. Found: C, 55.86; H, 4.66; N, 9.92.

### EXAMPLE 699

This example illustrates the preparation of 2-(2-{(E)-2-[4-(dimethylamino)-2-fluorophenyl]vinyl}pyridin-4-yl)-1,5,6,7-tetrahydro-4H-pyrrolo[3,2-c]pyridin-4-one trifluoroacetate.

mp 190-195° C.; ¹H NMR (300 MHz, DMSO-d₆) δ 12.44 (br s, 1H), 8.52 (d, J=6.5 Hz, 1H), 8.40 (s, 1H), 7.93 (d, J=16.4 Hz, 1H), 7.84 (d, J=6.4 Hz, 1H), 7.63 (s, 1H), 7.55 (t, J=6.0 Hz, 1H), 7.29 (s, 1H), 7.06 (d, J=16.4 Hz, 1H), 6.70-6.55 (m, 2H), 3.50-3.40 (m, 2H), 3.03 (s, 6H), 3.00-2.89 (m, 2H), m/z 377 [M+H]⁺.

### EXAMPLE 700

This example illustrates the preparation of N,N-dimethyl-3-{(E)-2-[4-(4-oxo-4,5,6,7-tetrahydro-1H-pyrrolo[3,2-c]pyridin-2-yl)pyridin-2-yl]vinyl}benzamide trifluoroacetate.

mp 138-141° C.; ¹H NMR (300 MHz, DMSO-d₆) δ 12.44 (s, 1H), 8.64 (d, J=6.0 Hz, 1H), 8.40 (s, 1H), 7.98 (d, J=16.5 Hz, 1H), 7.87 (d, J=6.2 Hz, 1H), 7.74 (d, J=5.6 Hz, 1H), 7.70 (s, 1H), 7.60-7.50 (m, 2H), 7.47 (d, J=7.6 Hz, 1H), 7.36 (d, J=16.4 Hz, 1H), 7.28 (s, 1H), 3.47-3.43 (m, 2H), 3.05-2.95 (m, 8H); ESI-MS m/z 387 [M+H]⁺.

### EXAMPLE 701

Reserved.

### EXAMPLE 702

This example illustrates the preparation of 2-(2-{(E)-2-[4-(diethylamino)phenyl]vinyl}pyridin-4-yl)-1,5,6,7-tetrahydro-4H-pyrrolo[3,2-c]pyridin-4-one trifluoroacetate. mp 129-131° C.; ¹H NMR (300 MHz, DMSO-d₆) δ 12.43 (s, 1H), 8.50 (d, J=6.5 Hz, 1H), 8.37 (s, 1H), 7.89 (d, J=16.1 Hz, 1H), 7.78 (dd, J=6.5, 1.4 Hz, 1H), 7.60 (d, J=1.9 Hz, 1H), 7.50 (d, J=8.9 Hz, 2H), 7.29 (s, 1H), 6.92 (d, J=16.2 Hz, 1H), 6.78 (d, J=8.8 Hz, 2H), 3.46-3.40 (m, 6H), 2.94 (t, J=6.8 Hz, 2H), 1.13 (t, J=7.0 Hz, 6H); ESI-MS m/z 387 [M+H]⁺.

### EXAMPLE 703

This example illustrates the preparation of N,N-dimethyl-2-{(E)-2-[4-(4-oxo-4,5,6,7-tetrahydro-1H-pyrrolo[3,2-c]pyridin-2-yl)pyridin-2-yl]vinyl}benzamide trifluoroacetate.

mp 150-153° C.; ¹H NMR (300 MHz, DMSO-d₆) δ 12.57 (s, 1H), 8.89 (d, J=5.2 Hz, 1H), 8.35 (s, 1H), 8.17 (d, J=7.5 Hz, 1H), 8.03-7.98 (m, 2H), 7.87-7.78 (m, 2H), 7.65-7.52 (m, 4H), 3.39 (d, J=5.1 Hz, 3H), 3.30-3.15 (m, 2H), 3.07 (d, J=5.1 Hz, 3H); ESI-MS m/z 387 [M+H]⁺.

### EXAMPLE 704

This example illustrates the preparation of 2-{2-[(E)-2-(1-methyl-2,3-dihydro-1H-indol-5-yl)vinyl]pyridin-4-yl}-1,5,6,7-tetrahydro-4H-pyrrolo[3,2-c]pyridin-4-one.

Step 1: (Preparation of 1-methyl-2,3-dihydro-1H-indole).

To a solution of 1-methylindole (3.9 mL, 30 mmol) in acetic acid (150 mL) at 10° C. was added NaBH₄ (7.0 g, 180 mmol) in small portions over 30 min. The cold bath was removed, and the reaction mixture was stirred at room temperature for 3.5 h, and then heated at 60° C. for 2.5 h. The cooled reaction mixture was poured into a solution of 150 g of Na₂CO₃ in 1000 mL of water, and the product was extracted into Et₂O (6×100 mL). The organic extract was washed with brine, dried (Na₂SO₄), filtered and concentrated. The crude product was dissolved in CH₂Cl₂ (100 mL), re-dried (Na₂SO₄), filtered through a plug of MgSO₄, followed by a plug of silica gel, and concentrated under reduced pressure to give 1-methyl-2,3-dihydro-1H-indole (3.33 g, 82%) as a yellow oil, which was used in step 2 without further purification: ¹H NMR (300 MHz, CDCl₃) δ 7.11-7.06 (m, 2H), 6.67 (t, J=7.3 Hz, 1H), 6.49 (d, J=8.0 Hz, 1H), 3.29 (t, J=8.2 Hz, 2H), 2.94 (t, J=8.1 Hz, 2H), 2.75 (s, 3H).

Step 2: (Preparation of 1-methyl-2,3-dihydro-1H-indole-5-carbaldehyde).

To ice-cold DMF (7.6 mL) was added POCl₃ (1.1 mL, 12.1 mmol) dropwise. After 5 min, a solution of 1-methyl-2,3-dihydro-1H-indole (1.5 g, 11.3 mmol) in DMF (3.2 mL) was added dropwise to the mixture. After 20 min, the ice-bath was removed, and the reaction mixture was stirred at room temperature for 1 h, and then at 80° C. for 20 min. The cooled reaction mixture was quenched with water (20 mL, added dropwise), and the product was extracted into Et₂O (3×50 mL). The Et₂O extract was washed with brine (25 mL), dried (Na₂SO₄), filtered and concentrated under reduced pressure. Purification by flash column chromatography (eluent 90:10 hexanes/EtOAc to 75:25 hexanes/EtOAc) gave 1-methyl-2,3-dihydro-1H-indole-5-carbaldehyde (1.39 g, 76%): ¹H NMR (300 MHz, CDCl₃) δ 9.67 (s, 1H), 7.56 (d, J=8.1 Hz, 1H), 7.55 (s, 1H), 6.38 (d, J=8.1 Hz, 1H), 3.55 (t, J=8.4 Hz, 2H), 3.03 (t, J=8.4 Hz, 2H), 2.88 (s, 3H).

Step 3: (Preparation of vinyl boronate intermediate).

The compound was prepared in 22% yield by a procedure similar to the one described in Example 529.

Step 4: (Preparation of 2-{2-[(E)-2-(1-methyl-2,3-dihydro-1H-indol-5-yl)vinyl]pyridin-4-yl}-1,5,6,7-tetrahydro-4H-pyrrolo[3,2-c]pyridin-4-one).

The above compound was prepared in 16% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described in Example 551: mp 289° C. (dec.); ¹H NMR (300 MHz, DMSO-d₆) δ 11.90 (br s, 1H), 8.41 (d, J=5.2 Hz, 1H), 7.73 (s, 1H), 7.67 (d, J=16.0 Hz, 1H), 7.39 (s, 2H), 7.06-7.02 (m, 2H), 6.95 (d, J=16.0 Hz, 1H), 6.51 (d, J=8.0 Hz, 1H), 6.51 (d, J=8.0 Hz, 1H), 3.42-3.31 (m, 4H), 2.95-2.93 (m, 2H), 2.85 (t, J=6.8 Hz, 2H), 2.75 (s, 3H); ESI-MS m/z 371 [M+H]⁺.

### EXAMPLE 705

This example illustrates the preparation of 2-(2-{(E)-2-[2-(1,3-thiazol-2-yl)phenyl]vinyl}pyridin-4-yl)-1,5,6,7-tetrahydro-4H-pyrrolo[3,2-c]pyridin-4-one trifluoroacetate.

Step 1: (Preparation of 2-thiazol-2-yl-benzaldehyde).

A mixture of 2-bromothiozole (1.64 g, 10 mmol)), 2-formylphenyl boronic acid (1.50 g, 10 mmol), toluene (20 mL) and aqueous 2 M Na₂CO₃ (8 mL) was degassed (2×, vacuum/argon). To this mixture was added PdCl₂(dppf)-CH₂Cl₂ (408 mg, 0.5 mmol ). The resulting mixture was degassed (3×, vacuum/argon) and then heated to reflux for 4 h. The cooled reaction mixture was diluted with EtOAc (200 mL), washed with water, dried (Na₂SO₄) and concentrated under reduced pressure. Purification by flash chromatography (4:1 hexanes/EtOAc) gave 2-thiazole-2-yl-benzaldehyde as an oil (450 mg, 24%). ESI-MS m/z 190 [M+H]⁺.

Step 2: (Preparation of vinyl boronate intermediate).

The compound was prepared in 23% yield by a procedure similar to the one described for the synthesis of Example 529.

Step 3: (Preparation of 2-(2-{(E)-2-[2-(1,3-thiazol-2-yl)phenyl]vinyl}pyridin-4-yl)-1,5,6,7-tetrahydro-4H-pyrrolo[3,2-c]pyridin-4-one trifluoroacetate ).

The above compound was prepared in 19% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551. mp 198-205° C.; ¹H NMR (300 MHz, CD₃OD-d₆) δ 8.48 (d, J=6.5 Hz, 1H), 8.35 (d, J=16.3 Hz, 1H), 8.21 (s, 1H), 8.05 (d, J=3.3 Hz, 1H), 7.99 (d, J=6.5 Hz, 1H), 7.90-7.80 (m, 3H), 7.72-7.60 (m, 2H), 7.49 (s, 1H), 7.28 (d, J=16.3 Hz, 1H), 3.64 (t, J=7.0 Hz, 2H), 3.04 (t, J=7.0 Hz, 2H); ESI-MS m/z 399 [M+H]⁺.

### EXAMPLE 706

This example illustrates the preparation of 2-(2-{(E)-2-[2-(3-furyl)phenyl]vinyl}pyridin-4-yl)-1,5,6,7-tetrahydro-4H-pyrrolo[3,2-c]pyridin-4-one trifluoroacetate.

Step 1 (Preparation of 2-furan-3-yl-benzaldehyde).

A mixture of 2-bromobenzaldehyde (2.77 g, 15.0 mmol), 3-furanboronic acid (1.85 g, 16.5 mmol), 2 N Na₂CO₃ (50 mL) and PdCl₂ddf CH₂Cl₂ (653 mg, 0.8 mmol) in toluene (120 mL) was degassed (3×, vacuum/argon) and heated to reflux for 12 h. The cooled reaction mixture was diluted with EtOAc (300 mL). The organic layer was washed with brine and concentrated under reduced pressure. Purification by flash column chromatography (eluent 2:3 CH₂Cl₂/hexanes) gave 2-furan-3-yl-benzaldehyde (1.2 g, 69%) as an oil: ¹H NMR (300 MHz, CDCl₃-d₆) δ 10.22.(d, J=0.5 Hz, 1H), 7.99 (dd, J=8.4, 6.7 Hz, 1H), 7.62-7.59 (m, 1H), 7.56-7.54 (m, 2H), 7.48-7.44 (m, 2H), 6.60-6.58 (m, 1H).

Step 2: (Preparation of vinyl boronate intermediate).

The above compound was prepared in 52% yield by a procedure similar to the one described for the synthesis of Example 529 using 2-furan-3-yl-benzaldehyde obtained in step 1.

Step 3: (Preparation of 2-(2-{(E)-2-[2-(3-furyl)phenyl]vinyl}pyridin-4-yl)-1,5,6,7-tetrahydro-4H-pyrrolo[3,2-c]pyridin-4-one trifluoroacetate).

The above compound was prepared in 2% yield by the cross coupling of the vinylboronate from step 2 above and 2-(2-chloropyridin-4-yl)-1,5,6,7-tetrahydro-4H-pyrrolo[3,2-c]pyridin-4-one according to the general procedure described for Example 551. mp 155-159° C.; ¹H NMR (300 MHz, CD₃OD) δ 8.42.(d, J=6.5 Hz, 1H), 8.19 (d, J=1.7 Hz, 1H), 8.04 (d, J=16.4 Hz, 1H), 7.86 (t, J=7.1 Hz, 2H), 7.67 (t, J=2.1 Hz, 2H), 7.52-7.48 (m, 4H), 7.22 (d, J=16.3 Hz, 1H), 6.69 (d, J=0.8 Hz, 1H), 3.61 (t, J=6.9 Hz, 2H), 3.01 (t, J=6.9 Hz, 2H); ESI-MS m/z 382 [M+H]⁺.

### EXAMPLE 707

This example illustrates the preparation of 2-{2-[(E)-2-(4-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 4-morpholin-4-yl-benzylaldehyde).

A mixture of 4-fluorobenzadehyde (3.68 g, 29.7 mmol), morpholine (2.84 g, 32.6 mmol) and K₂CO₃ (4.50 g, 32.6 mmol) in DMF (20 mL) was heated overnight at 130° C. The cooled reaction mixture was diluted with EtOAc (200 mL), washed with water (3×), dried (Na₂SO₄) and concentrated under reduced pressure. Purification by flash chromatography (eluent 3:1 hexanes/EtOAc) gave 4-morpholin-4-yl-benzylaldehyde (5.1 g, 90%) as a yellow solid. ¹H NMR (300 MHz, CDCl₃) δ 9.81 (s, 1H), 7.78 (d, J=8.7 Hz, 2H), 6.93 (d, J=8.7 Hz, 2H), 3.87 (t, J=4.8 Hz, 4H), 3.36 (t, J=4.8 Hz, 4H).

Step 2: (Preparation of vinyl boronate intermediate).

To an ice-cold solution of 2,2,6,6-tetramethylpiperidine (1.70 mL, 10 mmol) and N,N,N,N′-tetramethyethylenediamine (2.09 mL, 10 mmol) in anhydrous THF (12 mL), was added n-BuLi (4.40 mL, 11 mmol, 2.5 M solution in hexanes) dropwise. The yellow solution was stirred for 15 min followed by the addition of compound 1 (2.35 mL, 11 mmol). This solution was stirred for 30 min, then, a solution of 4-morpholin-4-yl-benzylaldehyde e (1.91 g, 10 mmol) in THF (4 mL) was added. The mixture was warmed up to room temperature and stirred for overnight. The resulting solution was cooled in ice-bath, diluted with EtOAc (20 mL) and treated with 1 N aqueous HCl (30 mL). The biphasic mixture was stirred for 30 min and carefully neutralized with 1 N NaOH. An additional amount of EtOAc (200 mL) was added for extraction. The organic phase was isolated and washed with water, brine, dried (Na₂SO₄) and concentrated under reduced pressure. Purification by flash column chromatography (eluent 3:1 hexanes/EtOAc) gave the styrenyl pinacolboranate (a mixture of the E and Z isomer, 1.08 g, 36%) as a yellow oil.

Step 3: (Preparation of 2-{2-[(E)-2-(4-morpholin-4-ylphenyl)vinyl]pyridin-4-yl}-1,5,6,7-tetrahydro-4H-pyrrolo[3,2-c]pyridin-4-one trifluoroacetate ).

This compound was prepared in 7% yield by the cross coupling of the vinylboronate from step 2 above and 2-(2-chloropyridin-4-yl)-1,5,6,7-tetrahydro-4H-pyrrolo[3,2-c]pyridin-4-one according to the general procedure described for Example 551: mp 205-210° C.; ¹H NMR (300 MHz, DMSO-d₆) δ 12.42 (br s, 1H), 8.55 (d, J=6.5 Hz, 1H), 8.42 (s, 1H), 7.92 (d, J=16.2 Hz, 1H), 7.84 (d, J=5.1 Hz, 1H), 7.63 (s, 1H), 7.55 (d, J=8.8 Hz, 2H), 7.30 (s, 1H), 7.10-7.00 (m, 3H), 3.85-3.70 (m, 4H), 3.51-3.39 (m, 2H), 3.38-3.28 (m, 4H), 3.00-2.90 (m, 2H); ESI-MS m/z 401 [M+H]⁺.

### EXAMPLE 708

This example illustrates the preparation of 2-{2-[(E)-2-(4-thiomorpholin-4-ylphenyl)vinyl]pyridin-4-yl}-1,5,6,7-tetrahydro-4H-pyrrolo[3,2-c]pyridin-4-one trifluoroacetate.

This compound was prepared according to the protocol described in Example 707 by replacing morpholine with thiomorpholine in step 1. mp 188-190° C.; ¹H NMR (300 MHz, DMSO-d₆) δ 12.42 (br s, 1H), 8.55 (d, J=6.5 Hz, 1H), 8.42 (s, 1H), 7.92 (d, J=16.2 Hz, 1H), 7.84 (d, J=5.1 Hz, 1H), 7.63 (s, 1H), 7.55 (d, J=8.8 Hz, 2H), 7.30 (s, 1H), 7.10-7.00 (m, 3H), 3.85-3.70 (m, 4H), 3.38-3.28 (m, 4H), 3.10-3.22 (m, 2H), 3.00-2.90 (m, 2H); ESI-MS m/z 417 [M+H]⁺.

### EXAMPLE 709

This example illustrates the preparation of 2-{2-[(E)-2-(2-chloro-6-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 2-Chloro-6-morpholin-4-yl-benzaldehyde).

This compound was prepared in 79% yield by a procedure similar to the one described in step 1 of the synthesis of Example 707 using morpholine and 2-chloro-6-fluorobenzaldehyde. ¹H NMR (300 MHz, CDCl₃) δ 10.36 (s, 1H), 7.41 (t, J=8.1 Hz, 1H), 7.12 (dd, J=7.1, 0.8 Hz, 1H), 7.03 (dd, J=7.7, 0.7 Hz, 1H), 3.89 (t, J=4.6 Hz, 4H), 3.07 (t, J=4.6 Hz, 4H).

Step 2: (Preparation of vinyl boronate intermediate).

The above compound was prepared from 2-chloro-6-morpholin-4-yl-benzaldehyde from step 1 using the general procedure described in step 2 of Example 707.

Step 3: (Preparation of 2-{2-[(E)-2-(2-chloro-6-morpholin-4-ylphenyl)vinyl]pyridin-4-yl}-1,5,6,7-tetrahydro-4H-pyrrolo[3,2-c]pyridin-4-one trifluoroacetate).

This compound was prepared in 3% yield by the cross coupling of the vinylboronate from step 2 above and 2-(2-chloropyridin-4-yl)-1,5,6,7-tetrahydro-4H-pyrrolo[3,2-c]pyridin-4-one according to the general procedure described in Example 551: mp 196-199° C.; ¹H NMR (300 MHz, DMSO-d₆) δ 12.31.(s, 1H), 8.60 (d, J=6.0 Hz, 1H), 8.25 (s, 1H), 7.93 (d, J=16.6 Hz, 1H), 7.81 (d, J=4.7 Hz, 1H), 7.59 (d, J=16.7 Hz, 1H), 7.48 (s, 1H), 7.36 (t, J=7.9 Hz, 1H), 7.27 (s, 1H), 7.23 (d, J=4.4 Hz, 1H), 7.14 (d, J=7.8 Hz, 1H), 3.80-3.50 (m, 4H), 3.46-3.35 (m, 2H), 2.96-2.83 (m, 6H); ESI-MS m/z 435 [M+H]⁺.

### EXAMPLE 710

This example illustrates the preparation of 2-(2-{(E)-2-[2-(dipropylamino)phenyl]vinyl}pyridin-4-yl)-1,5,6,7-tetrahydro-4H-pyrrolo[3,2-c]pyridin-4-one trifluoroacetate.

Step 1: (Preparation of 2-N,N-dipropylaminobenzylaldehyde).

A mixture of 2-fluorobenzadehyde (5.85 g, 47.1 mmol), dipropylamine (5.48 g, 54.2 mmol) and K₂CO₃ (7.49 g, 54.2 mmol) in DMF (47 mL) was heated overnight at 152° C. The cooled reaction mixture was diluted with water (90 mL) and extracted with EtOAc (3×70 mL). The combined EtOAc extracts were then dried (Na₂SO₄) and concentrated under reduced pressure. Purification by flash chromatography (eluent 97:3 hexanes/EtOAc) gave 2-N,N-dipropylaminobenzylaldehyde (2.73 g, 28%) as a yellow solid: ¹H NMR (300 MHz, CDCl₃) δ 10.31 (s, 1H), 7.74 (dd, J=7.8, 1.7 Hz, 1H), 7.57-7.51 (m, 1H), 7.28 (d, J=8.3 Hz, 1H), 7.09 (t, J=7.1 Hz, 1H), 3.13 (t, J=7.4 Hz, 4H), 1.55-1.48 (m, 4H), 0.86 (t, J=7.4 Hz, 6H).

Step 2: (Preparation of vinyl boronate intermediate).

The above compound was prepared in 21% yield from 2-N,N-dipropylaminobenzylaldehyde from step 1 using the general procedure described in step 2 of Example 707.

Step 3: (Preparation of 2-(2-{(E)-2-[2-(dipropylamino)phenyl]vinyl}pyridin-4-yl)-1,5,6,7-tetrahydro-4H-pyrrolo[3,2-c]pyridin-4-one trifluoroacetate ).

This compound was prepared in 9% yield by the cross coupling of the vinylboronate from step 2 above and 2-(2-chloropyridin-4-yl)-1,5,6,7-tetrahydro-4H-pyrrolo[3,2-c]pyridin-4-one according to the general procedure described in Example 551: mp 160-163° C.; ¹H NMR (300 MHz, DMSO-d₆) δ 12.73 (s, 1H), 8.58 (d, J=6.2 Hz, 1H), 8.39 (s, 1H), 8.16 (d, J=15.4 Hz, 1H), 7.91 (d, J=5.3 Hz, 1H), 7.66 (d, J=7.7 Hz, 1H), 7.50 (s, 1H), 7.39-7.15 (m, 5H), 3.46-3.42 (m, 2H), 2.98-3.10 (m, 4H), 2.93 (t, J=6.7 Hz, 2H), 1.47-1.40 (m, 4H), 0.80 (t, J=7.3 Hz, 6H); ESI-MS m/z 415 [M+H]⁺.

### EXAMPLE 711

This example illustrates the preparation of 2-(2-{(E)-2-[2-(diethylamino)phenyl]vinyl}pyridin-4-yl)-1,5,6,7-tetrahydro-4H-pyrrolo[3,2-c]pyridin-4-one trifluoroacetate.

Step 1: (Preparation of 2-N,N-diethylaminobenzylaldehyde).

2-N,N-Diethylaminobenzylaldehyde was prepared in 88% yield by a procedure similar to the one described in step 1 in the synthesis of Example 710 using 2-fluorobenzaldehyde and diethylamine: ¹H NMR (300 MHz, CDCl₃) δ 10.32 (s, 1H), 7.75 (dd, J=7.7, 1.7 Hz, 1H), 7.59-7.52 (m, 1H), 7.29 (d, J=8.1 Hz, 1H), 7.12 (t, J=7.5 Hz, 1H), 3.20 (q, J=7.1 Hz, 4H), 1.05 (t, J=7.1 Hz, 6H).

Step 2: (Preparation of vinyl boronate intermediate).

The above compound was prepared in 7% yield from 2-N,N-diethylaminobenzylaldehyde from step 1 using the general procedure described in step 2 of Example 707.

Step 3: (Preparation of 2-(2-{(E)-2-[2-(diethylamino)phenyl]vinyl}pyridin-4-yl)-1,5,6,7-tetrahydro-4H-pyrrolo[3,2-c]pyridin-4-one trifluoroacetate).

This compound was prepared in 4% yield by the cross coupling of the vinylboronate from step 2 above and 2-(2-chloropyridin-4-yl)-1,5,6,7-tetrahydro-4H-pyrrolo[3,2-c]pyridin-4-one according to the general procedure described in Example 551: mp 130-135° C.; ¹H NMR (300 MHz, CD₃OD) δ 8.44 (d, J=6.4 Hz, 1H), 8.28 (s, 1H), 8.26 (d, J=16.3 Hz, 1H), 7.84-7.77 (m, 2H), 7.50-7.46 (m, 2H), 7.36-7.23 (m, 3H), 3.61 (t, J=6.9 Hz, 2H), 3.26 (q, J=7.0 Hz, 4H), 3.03 (t, J=7.0 Hz, 2H), 1.05 (t, J=7.1 Hz, 6H); ESI-MS m/z 387 [M+H]⁺.

### EXAMPLE 712

This example illustrates the preparation of 2-(2-{(E)-2-[4-(pyrrolidin-1-ylcarbonyl)phenyl]vinyl}pyridin-4-yl)-1,5,6,7-tetrahydro-4H-pyrrolo[3,2-c]pyridin-4-one trifluoroacetate.

Step 1: (Preparation of 4-(pyrrolidine-1-carbonyl)-benzaldehyde).

To a solution of 4-carboxybenzaldehyde (2.46 g, 15.0 mmol) in anhydrous DMF (75 mL), was added EDCl (5.75 g, 30.0 mmol) and HOBt (3.04 g, 22.5 mmol). The resulting solution was stirred for 30 min followed by the addition of pyrrolidine. The reaction mixture was stirred for 1 h at room temperature, and then reaction mixture was diluted with EtOAc (150 mL) and water (75 mL). The aqueous layer was extracted with additional amount of EtOAc (2×75 mL). The combined organic phase was washed with water (2×50 mL), saturated solution of Na₂CO₃ (3×50 mL), brine, dried (Na₂SO₄) and concentrated under reduced pressure. Purification by flash chromatography (eluent 7:3 EtOAc/hexanes) gave the desire amide (1.91 g, 63%) as a solid; ¹H NMR (300 MHz, CDCl₃) δ 10.06 (s, 1H), 7.93 (d, J=8.1 Hz, 2H), 7.67 (d, J=8.1 Hz, 2H), 3.67 (t, J=6.7 Hz, 2H), 3.39 (t, J=6.7 Hz, 2H), 1.99-1.91 (m, 4H).

Step 2: (Preparation of vinyl boronate intermediate).

The above compound was prepared in 62% yield from 4-(pyrrolidine-1-carbonyl)-benzaldehyde from step 1 using the general procedure described in step 2 of Example 707.

Step 3: (Preparation of 2-(2-{(E)-2-[4-(pyrrolidin-1-ylcarbonyl)phenyl]vinyl}pyridin-4-yl)-1,5,6,7-tetrahydro-4H-pyrrolo[3,2-c]pyridin-4-one trifluoroacetate).

This compound was prepared in 16% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 218-222° C.; ¹H NMR (300 MHz, DMSO-d₆) δ 12.36 (s, 1H), 8.62 (d, J=6.1 Hz, 1H), 8.33 (s, 1H), 7.95 (d, J=16.4 Hz, 1H), 7.81 (d, J=5.8 Hz, 1H), 7.73 (d, J=8.2 Hz, 2H), 7.63 (d, J=8.2 Hz, 2H), 7.49 (s, 1H), 7.34 (d, J=16.3 Hz, 1H), 7.26 (s, 1H), 3.55-3.43 (m, 6H), 2.92 (t, J=7.1 Hz, 2H), 1.90-1.80 (m, 4H); ESI-MS m/z 413 [M+H]⁺.

### EXAMPLE 713

This example illustrates the preparation of 2-{2-[(E)-2-(4-pyrrolidin-1-ylphenyl)vinyl]pyridin-4-yl}-1,5,6,7-tetrahydro-4H-pyrrolo[3,2-c]pyridin-4-one trifluoroacetate.

Step 1: (Preparation of 4-pyrrolidin-1-yl-benzaldehyde).

A mixture of 4-fluorobenzaldehyde (3.68 g, 29.7 mmol), pyrrolidine (2.31 g, 32.6 mmol) and K₂CO₃ (4.50 g, 32.6 mmol) in DMF (20 mmol) was heated for 14 h at 130° C. The resulting mixture was diluted with EtOAc (200 mL), washed with water, dried (Na₂SO₄) and concentrated under reduced pressure. Purification by flash chromatography (3:1 hexanes/EtOAc) gave 4-pyrrolidin-1-yl-benzaldehyde as a yellow solid (4.3 g, 83%). ¹H NMR (300 MHz, CDCl₃) δ 9.70 (s, 1H), 7.72 (d, J=8.7 Hz, 2H), 6.57 (d, J=8.7 Hz, 2H), 3.39 (t, J=6.6 Hz, 4H), 2.15-1.90 (m, 4H).

Step 2: (Preparation of vinyl boronate intermediate).

The above compound was prepared in 54% yield by a procedure similar to the one described in step 2 of the synthesis in Example 707 using 4-pyrrolidin-1-yl-benzaldehyde obtained in step 1.

Step3: (Preparation of 2-{2-[(E)-2-(4-pyrrolidin-1-ylphenyl)vinyl]pyridin-4-yl}-1,5,6,7-tetrahydro-4H-pyrrolo[3,2-c]pyridin-4-one trifluoroacetate).

This compound was prepared in 7% yield by the cross coupling of the vinylboronate from step 2 above and 2-(2-chloropyridin-4-yl)-1,5,6,7-tetrahydro-4H-pyrrolo[3,2-c]pyridin-4-one according to the general procedure described for Example 551: mp 209-212° C.; ¹H NMR (300 MHz, DMSO-d₆) δ 12.50 (br s, 1H), 8.50 (d, J=6.5 Hz, 1H), 8.34 (s, 1H), 7.89 (d, J=16.2 Hz, 1H), 7.76 (d, J=6.6 Hz, 1H), 7.58 (s, 1H), 7.51 (d, J=8.5 Hz, 2H), 7.28 (s, 1H), 6.92 (d, J=16.2 Hz, 1H), 6.65 (d, J=8.5 Hz, 2H), 3.51-3.39 (m, 2H), 3.39-3.29 (m, 4H), 3.00-2.89(m, 2H), 2.05-1.90 (m, 4H); ESI-MS m/z 385 [M+H]⁺.

### EXAMPLE 714

This example illustrates the preparation of 2-{2-[(E)-2-(2,4-dimorpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 2,4-di-morpholin-4-yl-benzaldehyde).

To a solution of 4-bromo-2-fluorobenzaldehyde (11.3 g, 55.4 mmol) and morpholine (5.6 mL, 63.7 mmol) in DMF (56 mL) was added anhydrous K₂CO₃ (8.81 g, 63.7 mmol) and the resulting mixture was heated at 152° C. overnight. The reaction mixture was concentrated under reduced pressure to a smaller volume and diluted with CH₂Cl₂ (150 mL) and H₂O (15 mL). The aqueous phase was extracted with additional amount of CH₂Cl₂ (3×150 mL). The combined organic phase was dried (Na₂SO₄) and concentrated under reduced pressure. Purification by flash chromatography (eluent, 9:1 hexanes/EtOAc to 8.5:0.5:1 to 3:1:1 to 4:0.5:0.5 CH₂Cl₂/hexanes/EtOAc) gave 4-bromo-2-morpholin-4-yl-benzaldehyde (11.26 g, 75%). ¹H NMR (300 MHz, CDCl₃) δ 10.23 (d, J=0.2 Hz, 1H), 7.66 (d, J=8.2 Hz, 1H), 7.31-7.24 (m, 2H), 3.92-3.85 (m, 4H), 3.12 (m, 4H). Further eluting gave 2,4-di-morpholin-4-yl-benzaldehyde (2.8 g, 18%). ¹H NMR (300 MHz, CDCl₃) δ 10.07 (s, 1H), 7.73 (d, J=8.7 Hz, 1H), 6.60 (dd, J=8.7, 2.2 Hz, 1H), 6.40 (d, J=2.2 Hz, 1H), 3.94-3.80 (m, 8H), 3.33 (t, J=4.9 Hz, 4H), 3.13-3.04 (m, 4H).

Step 2: (Preparation of vinyl boronate intermediate).

The above compound was prepared in 42% yield by a procedure similar to the one described in step 2 of the synthesis in Example 707 using 2,4-di-morpholin-4-yl-benzaldehyde obtained in step 1.

Step 3: (Preparation of 2-{2-[(E)-2-(2,4-dimorpholin-4-ylphenyl)vinyl]pyridin-4-yl}-1,5,6,7-tetrahydro-4H-pyrrolo[3,2-c]pyridin-4-one trifluoroacetate).

This compound was prepared in 26% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 205-210° C.; ¹H NMR (300 MHz, DMSO-d₆) δ 12.52 (s, 1H), 8.53 (d, J=6.5 Hz, 1H), 8.37 (s, 1H), 7.97 (d, J=16.2 Hz, 1H), 7.85 (d, J=6.4 Hz, 1H), 7.61 (s, 1H), 7.52 (d, J=8.7 Hz, 1H), 7.31 (s, 1H), 7.16 (d, J=16.2 Hz, 1H), 6.76 (d, J=8.6 Hz, 1H), 6.62 (s, 1H), 3.84-3.72 (m, 8H) 3.46-3.41 (m, 2H), 3.27 (s, 4H), 2.94 (s, 6H); ESI-MS m/z 486 [M+H]⁺.

### EXAMPLE 715

This example illustrates the preparation of 2-{2-[(E)-2-(2-fluoro-6-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 2-Flouro-6-morpholin-4-yl-benzaldehyde).

2-Flouro-6-morpholin-4-yl-benzaldehyde was prepared in 66% yield by a procedure similar to the one described in step 1 of the synthesis of Example 707 using morpholine and 2,6-difluorobenzaldehyde: ¹H NMR (300 MHz, CDCl₃) δ 10.32 (s, 1H), 7.55-7.42 (m, 1H), 6.88-6.72 (m, 2H), 3.90-3.85 (m, 4H), 3.15-3.05 (m, 4H).

Step 2: (Preparation of vinyl boronate intermediate).

The above compound was prepared in 48% yield by a procedure similar to the one described in step 2 of the synthesis of Example 707 using 2-Flouro-6-morpholin-4-yl-benzaldehyde obtained in step 1.

Step 3: (Preparation of 2-{2-[(E)-2-(2-fluoro-6-morpholin-4-ylphenyl)vinyl]pyridin-4-yl}-1,5,6,7-tetrahydro-4H-pyrrolo[3,2-c]pyridin-4-one trifluoroacetate).

This compound was prepared in 5% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 143-147° C.; ¹H NMR (300 MHz, DMSO-d₆) δ 12.44 (s, 1H), 8.60 (d, J=6.2 Hz, 1H), 8.37 (s, 1H), 7.95-7.82 (m, 2H), 7.57 (s, 1H), 7.47-7.39 (m, 2H), 7.28 (s, 1H), 7.10-6.95 (m, 2H), 3.90-3.80 (m, 4H), 3.45 (t, J=6.5 Hz, 2H), 3.00-2.80 (m, 6H); ESI-MS m/z 419 [M+H]⁺.

### EXAMPLE 716

This example illustrates the preparation of 2-{2-[(E)-2-(2-thiomorpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 2-thiomorpholin-4-yl-benzaldehyde). 2-Thiomorpholin-4-yl-benzaldehyde was prepared in 66% yield by a procedure similar to the one described in step 1 of the synthesis of Example 707 using thiomorpholine and 2-fluorobenzaldehyde. ¹H NMR (300 MHz, CDCl₃) δ 10.32 (d, J=0.4 Hz, 1H), 7.82 (dd, J=6.0, 1.6 Hz, 1H), 7.57-7.50 (m, 1H), 7.19-7.10 (m, 2H), 3.40-3.25 (m, 4H), 2.90-2.75 (m, 4H).

Step 2: (Preparation of vinyl boronate intermediate). The above compound was prepared in 68% yield by a procedure similar to the one described in step 2 of the synthesis of Example 707 using 2-thiomorpholin-4-yl-benzaldehyde obtained in step 1.

Step 3: (Preparation of 2-{2-[(E)-2-(2-thiomorpholin-4-ylphenyl)vinyl]pyridin-4-yl}-1,5,6,7-tetrahydro-4H-pyrrolo[3,2-c]pyridin-4-one trifluoroacetate):

This compound was prepared in 5% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 173-177° C.; ¹H NMR (300 MHz, DMSO-d₆) δ 12.47 (s, 1H), 8.61 (d, J=6.2 Hz, 1H), 8.34 (s, 1H), 8.06 (d, J=16.1 Hz, 1H), 7.85 (d, J=6.1 Hz, 1H), 7.66 (d, J=7.6 Hz, 1H), 7.52 (s, 1H), 7.41 (t, J=6.9 Hz, 1H), 7.35-7.15 (m, 4H), 3.45 (t, J=6.5 Hz, 2H), 3.25-3.10 (m, 4H), 3.00-2.75 (m, 6H); ESI-MS m/z 417 [M+H]⁺.

### EXAMPLE 717

This example illustrates the preparation of 2-(2-{(E)-2-[2-(1-oxidothiomorpholin-4-yl)phenyl]vinyl}pyridin-4-yl)-1,5,6,7-tetrahydro-4H-pyrrolo[3,2-c]pyridin-4-one trifluoroacetate.

To a solution of the compound made in Example 716 (150 mg, 0.36 mmol) in MeOH (6 mL) was added p-toluensulfonylimidazole (64 mg, 0.29 mmol). To the above solution was added 2 N NaOH (0.124 mL), and H₂O₂ (0.013 mL, 32% in H₂O) was added at −20° C. The resulting reaction mixture was stirred at −20° C. for 20 min. The reaction mixture was concentrated and extracted with CH₂Cl₂. Purification by preparative column chromatography gave the title compound (a salt containing 1 equivalent of TFA, 158 mg, 81%) as a yellow solid. mp 169-172° C.; ¹H NMR (300 MHz, DMSO-d₆) δ 12.40 (s, 1H), 8.62 (d, J=6.2 Hz, 1H), 8.33 (s, 1H), 8.06 (d, J=16.4 Hz, 1H), 7.50 (d, J=5.6 Hz, 1H), 7.68 (d, J=7.8 Hz, 1H), 7.54 (s, 1H), 7.45-7.41 (m, 1H), 7.33-7.20 (m, 4H), 3.50-3.35 (m, 4H), 3.10-2.85 (m, 8H); ESI-MS m/z 433 [M+H]⁺.

### EXAMPLE 718

This example illustrates the preparation of 2-(2-{(E)-2-[2-(1,1-dioxidothiomorpholin-4-yl)phenyl]vinyl}pyridin-4-yl)-1,5,6,7-tetrahydro-4H-pyrrolo[3,2-c]pyridin-4-one trifluoroacetate.

To a solution of the compound made in Example 716 (130 mg, 0.31 mmol) in MeOH (6 mL), was added p-toluensulfonylimidazole (111 mg, 0.5 mmol). To the above solution was added 2 N NaOH (0.248 mL) and H₂O₂ (0.113 mL, 32% in H₂O) at −5° C. The mixture was stirred at rt for 2 h. The reaction mixture was concentrated and extracted with CH₂Cl₂. Purification by preparative column chromatography gave the title compound (a salt containing 1 equivalent of TFA, 64 mg, 46%) as a yellow solid: mp 196-199° C.; ¹H NMR (300 MHz, DMSO-d₆) δ 12.38 (br s, 1H), 8.64 (s, J=6.1 Hz, 1H) 8.29 (s, 1H), 8.09 (d, J=16.5 Hz, 1H), 7.82 (d, J=5.5 Hz, 1H), 7.69 (d, J=7.6 Hz, 1H), 7.51 (s, 1H), 7.42 (t, J=7.1 Hz, 1H), 7.42-7.20 (m, 4H), 3.50-3.30 (m, 10H), 2.92 (t, J=6.8 Hz, 2H); ESI-MS m/z 449 [M+H]⁺.

### EXAMPLE 719

This example illustrates the preparation of 2-[2-((E)-2-{2-[(2-methoxyethyl)(methyl)amino]phenyl}vinyl)pyridin-4-yl]-1,5,6,7-tetrahydro-4H-pyrrolo[3,2-c]pyridin-4-one trifluoroacetate.

Step 1: (Preparation of 2-[(2-Methoxyethyl)methylamino]benzaldehyde).

2-[(2-Methoxyethyl)methylamino]benzaldehyde was prepared in quantitative yield by a procedure similar to the one described in step 1 of the synthesis of Example 707 using 2-fluorobenzaldehyde and N-(2-methoxyethyl)methylamine. ¹H NMR (300 MHz, CDCl₃) δ 10.32 (s, 1H), 7.78 (dd, J=7.7, 1.6 Hz, 1H), 7.48 (td, J=7.7, 1.7 Hz, 1H), 7.14 (d, J=8.3 Hz, 1H), 7.05 (t, J=7.4 Hz, 1H), 3.58 (t, J=5.7 Hz, 2H), 3.34-3.32 (m, 5H), 2.94 (s, 3H).

Step 2: (preparation of vinyl boronate intermediate).

The above compound was prepared according to the procedure described in step 2 of the synthesis in Example 707 using 2-[(2-methoxyethyl)methylamino]-benzaldehyde. The crude product was used without purification in step 3.

Step 3: (Preparation of 2-[2-((E)-2-{2-[(2-methoxyethyl)(methyl)amino]phenyl}vinyl)pyridin-4-yl]-1,5,6,7-tetrahydro-4H-pyrrolo[3,2-c]pyridin-4-one trifluoroacetate).

This compound was prepared in 8% yield by the cross coupling of the vinylboronate from step 2 above and 2-(2-chloropyridin-4-yl)-1,5,6,7-tetrahydro-4H-pyrrolo[3,2-c]pyridin-4-one according to the general procedure described for Example 551: mp 180-183° C.; ¹H NMR (300 MHz, DMSO-d₆) δ 12.40 (br s, 1H), 8.59 (d, J=6.2 Hz, 1H), 8.31 (br s, 1H), 8.11 (d, J=16.4 Hz, 1H), 7.95-7.85 (m, 1H), 7.63 (dd, J=7.7, 1.3 Hz, 1H), 7.50 (br s, 1H), 7.38-7.30 (m, 1H), 7.22 (br s, 1H), 7.20-7.10 (m, 3H), 3.60-3.44 (m, 4H), 3.14 (s, 3H), 3.10 (t, J=5.8 Hz, 2H), 2.92 (t, J=6.8 Hz, 2H), 2.82 (s, 3H); ESI-MS m/z 403 [M+H]⁺.

### EXAMPLE 720

This example illustrates the preparation of 2-{2-[(E)-2-(4-{[(2S)-2-(methoxymethyl)pyrrolidin-1-yl]carbonyl}-2-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 4-{[(2S)-2-(methoxymethyl)pyrrolidin-1-yl]carbonyl}-2-morpholin-4-ylbenzaldehyde).

To a solution of 4-carboxy-2-morpholin-4-ylbenzaldehyde (0.85 g, 3.61 mmol) in DMF (18 mL), was added (S)-(+)-2-(methoxymethyl)pyrrolidine (0.5 g, 4.34 mmol), EDCl (1.38 g, 7.21 mmol), and HOBt (0.57, 4.26 mmol). The mixture was stirred for 30 min at room temperature. The reaction mixture was diluted with CH₂Cl₂ and H₂O. The aqueous phase was extracted with more CH₂Cl₂. The combined organic phase was dried (Na₂SO₄) and concentrated under reduced pressure. Purification by flash chromatography (eluent, 98:2 to 96:4 CH₂Cl₂/MeOH) gave the desired product (1.16 g, 97%): ¹H NMR (300 MHz, CDCl₃) δ 10.31 (s, 1H), 7.82 (d, J=7.7 Hz, 1H), 7.25-7.13 (m, 3H), 4.88-4.36 (m, 1H), 3.98-3.84 (m, 4H), 3.65 (d, J=4.4 Hz, 2H), 3.50-3.29 (m, 4H), 3.11 (t, J=4.5 Hz, 4H), 2.15-1.88 (m, 3H), 1.86-1.68 (m, 1H).

Step 2: (Preparation of vinyl boronate intermediate).

The above intermediate was prepared in 79% yield by a procedure similar to the one described in step 2 of the synthesis in Example 707 using 4-{[(2S)-2-(methoxymethyl)pyrrolidin-1-yl]carbonyl}-2-morpholin-4-ylbenzaldehyde obtained in step 1.

Step 3: (Preparation of 2-{2-[(E)-2-(4-{[(2S)-2-(methoxymethyl)pyrrolidin-1-yl]carbonyl}-2-morpholin-4-ylphenyl)vinyl]pyridin-4-yl}-1,5,6,7-tetrahydro-4H-pyrrolo[3,2-c]pyridin-4-one trifluoroacetate ).

This compound was prepared in 5% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 90-95° C.; ¹H NMR (300 MHz, DMSO-d₆) δ 12.39 (s, 1H), 8.62 (d, J=6.2 Hz, 1H), 8.38 (s, 1H), 8.03 (d, J=16.3 Hz, 1H), 8.93-8.82 (m, 1H), 7.69 (d, J=8.0 Hz, 1H), 7.57 (s, 1H), 7.38-7.16 (m, 4H), 4.30-4.12 (m, 1H), 3.83 (s, 4H), 3.69-3.51 (m, 1H), 3.50-3.19 (m, 6H), 2.98-2.89 (m, 8H), 2.10-1.58 (m, 4H); ESI-MS m/z 542 [M+H]⁺.

### EXAMPLE 721

This example illustrates the preparation of 2-(2-{(E)-2-[2,6-difluoro-4-(morpholin-4-ylcarbonyl)phenyl]vinyl}pyridin-4-yl)-1,5,6,7-tetrahydro-4H-pyrrolo[3,2-c]pyridin-4-one trifluoroacetate.

Step 1: (Preparation of 3,5-difluoro-4-formylbenzoic acid).

To a solution of 3,5-difluorobenzoic acid (10 g, 63.25 mmol) in THF (290 mL) at −78° C., was added dropwise a solution of t-BuLi (93 mL, 158.1 mmol, 1.7 M in pentane). The mixture was stirred for 30 min prior to the addition of DMF (12.3 mL, 158.18 mmol). The resulting solution was stirred at −78° C. for 1 h, at 0° C. for 1 h, then at room temperature for 30 min. The reaction was quenched by adding concentrated HCl till pH˜1, then, the solution was concentrated under reduced pressure to a smaller volume which was diluted with CH₂Cl₂ (200 mL). The aqueous layer was extracted with additional amount of CH₂Cl₂ (2×100 mL). The combined organic phase was concentrated and the residue was diluted with CH₂Cl₂ and washed with a solution of saturated Na₂CO₃. The combined organic phase was acidified till pH˜1 and extracted with CH₂Cl₂. The combined organic phase was concentrated under reduced pressure and the residue was triturated with a mixture of hexanes, ethyl acetate and CH₂Cl₂ to give 3,5-difluoro-4-formylbenzoic acid (2.01 g, 17%) as a tan solid: ¹H NMR (300 MHz, DMSO-d₆) δ 14.05 (s, 1H), 10.24 (s, 1H), 7.67 (d, J=9.1 Hz, 2H).

Step 2: (Preparation of 2,6-difluoro-4-(morpholin-4-ylcarbonyl)benzaldehyde).

The above intermediate was prepared in 90% yield following a procedure similar to the one used in step 1 of the synthesis of Example 720 using morpholine and the intermediate obtained in step 1. The residue obtained after the aqueous work-up was purified by flash chromatography (eluent 98:2 to 96:4 CH₂Cl₂/MeOH) to give the title compound (2.4 g, 90%).

Step 3: (Preparation of vinyl boronate intermediate).

The above intermediate was prepared in 73% yield by a procedure similar to the one described in step 2 of the synthesis in Example 707 using 2,6-difluoro-4-(morpholin-4-ylcarbonyl)benzaldehyde obtained in step 2.

Step 4: (Preparation of 2-(2-{(E)-2-[2,6-difluoro-4-(morpholin-4-ylcarbonyl)phenyl]vinyl}pyridin-4-yl)-1,5,6,7-tetrahydro-4H-pyrrolo[3,2-c]pyridin-4-one trifluoroacetate).

This compound was prepared in 1% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 205-210° C.; ¹H NMR (300 MHz, DMSO-d₆) δ 12.26 (s, 1H), 8.61 (d, J=5.8 Hz, 1H), 8.26 (s, 1H), 7.84 (d, J=16.6 Hz, 1H), 7.81-7.75 (m, 1H), 7.50 (d, J=16.6 Hz, 1H), 7.44 (s, 1H), 7.35 (d, J=8.9 Hz, 2H), 7.21 (s, 1H), 3.74-3.12 (m, 10H), 2.91 (t, J=6.7 Hz, 2H); ESI-MS m/z 465 [M+H]⁺.

### EXAMPLE 722

This example illustrates the preparation of 2-{2-[(E)-2-(4-fluoro-2-morpholin-4-ylphenyl)vinyl]pyridin-4-yl}-1,5,6,7-tetrahydro-4H-pyrrolo[3,2-c]pyridin-4-one.

Step 1: (Preparation of 4-fluoro-2-morpholin-4-yl-benzylaldehyde).

A mixture of 2,4-difluorobenzadehyde (5.68 g, 40 mmol), morpholine (3.48 g, 40 mmol) and K₂CO₃ (6.07 g, 44 mmol) in DMF (20 mL) was heated overnight at 130° C. The cooled reaction mixture was diluted with EtOAc, washed with water, dried (Na₂SO₄) and concentrated under reduced pressure. Purification by flash chromatography (eluent 4:1 hexanes/EtOAc) gave 4-fluoro-2-morpholin-4-yl-benzaldehyde (2.5 g, 30%) as a yellow solid: ¹H NMR (300 MHz, CDCl₃) δ 10.19 (s, 1H), 7.90-7.80 (m, 1H), 6.88-6.70 (m, 2H), 3.90 (t, J=4.8 Hz, 4H), 3.09 (t, J=4.8 Hz, 4H). Continuously eluting gave 2-fluoro-4-morpholin-4-yl-benzaldehyde (3.8 g, 45%) as a yellow solid ¹H NMR (300 MHz, CDCl₃) δ 10.12 (s, 1H), 7.75 (t, J=8.7 Hz, 1H), 6.68 (dd, J=9.0, 2.4 Hz, 1H), 6.48 (dd, J=14.1, 2.4 Hz, 1H), 3.85 (t, J=4.8 Hz, 4H), 3.34 (t, J=4.8 Hz, 4H).

Step 2: (Preparation of vinyl boronate intermediate).

The above intermediate was prepared in 43% yield by a procedure similar to the one described in step 2 of the synthesis in Example 707 using 4-fluoro-2-morpholin-4-yl-benzaldehyde obtained in step 1.

Step 3: (Preparation of 2-{2-[(E)-2-(4-fluoro-2-morpholin-4-ylphenyl)vinyl]pyridin-4-yl}-1,5,6,7-tetrahydro-4H-pyrrolo[3,2-c]pyridin-4-one).

This compound was prepared in 24% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: ¹H NMR (300 MHz, DMSO-d₆) δ 12.36 (br s, 1H), 8.58 (d, J=6.1 Hz, 1H), 8.26 (br s, 1H), 7.94 (d, J=16.4 Hz, 1H), 7.81 (s, 1H), 7.69 (t, J=8.0 Hz, 1H), 7.48 (s, 1H), 7.28-7.18 (m, 2H), 7.07-6.97 (m, 2H), 3.86-3.73 (m, 4H), 3.47-3.37 (m, 2H), 3.00-2.83 (m, 6H); m/z419 [M+H]⁺. Anal. Calculated for C₂₄H₂₃FN₄O₂-1.125CF₃CO₂: C, 57.67; H, 4.45; N, 10.24. Found: C, 57.93; H, 4.57; N, 10.18.

### EXAMPLE 723

This example illustrates the preparation of 2-(2-{(E)-2-[2-(4-hydroxypiperidin-1-yl)phenyl]vinyl}pyridin-4-yl)-1,5,6,7-tetrahydro-4H-pyrrolo[3,2-c]pyridin-4-one trifluoroacetate.

Step 1: (Preparation of 2-[4-hydroxypiperidin-1-yl]benzaldehyde).

The above compound was prepared by a procedure similar to the one described in step 1 of the synthesis of Example 707 using 2-fluorobenzaldehye and 4-hydroxypiperidine.

Step 2: (Preparation of 2-[4-(tert-butyldimethylsilanoxy)piperidine-1-yl]benzaldehyde).

To a solution of 2-(4-hydroxypiperidin-1-yl)benzaldehyde from step 1 (3.68 g, 17.9 mmol) and imidazole (2.66 g, 39.1 mmol) in DMF (40 mL) was added TBSCl (2.97 g, 19.7 mmol), and the mixture was stirred at room temperature overnight. The reaction mixture was poured into water (150 mL), and the product was extracted into EtOAc (3×100 mL). The organic extract was washed with water (3×50 mL), dried (Na₂SO₄), filtered and concentrated under reduced pressure. Purification by flash chromatography (eluent 90:10 to 80:20 hexanes/Et₂O) gave 2-[4-(tert-butyldimethylsilanoxy)piperidine-1-yl]benzaldehyde (3.19 g, 56%). ¹H NMR (300 MHz, CDCl₃) δ 10.20 (s, 1H), 7.71 (dd, J=7.7, 1.6 Hz, 1H), 7.41 (td, J=7.7, 1.6 Hz, 1H), 7.04-6.96 (m, 2H), 3.83-3.81 (m, 1H), 3.20-3.16 (m, 2H), 2.90-2.85 (m, 2H), 1.85-1.82 (m, 2H), 1.70-1.68 (m, 2H), 0.82 (s, 9H), 0.00 (s, 6H).

Step 3: (Preparation of vinyl boronate intermediate).

The above intermediate was prepared in 55% yield by a procedure similar to the one described in step 2 of the synthesis of Example 707 using 2-[4-(tert-butyldimethylsilanoxy)piperidine-1-yl]benzaldehyde obtained in step 2.

Step 4: (Preparation of 2-(2-{(E)-2-[2-(4-hydroxypiperidin-1-yl)phenyl]vinyl}pyridin-4-yl)-1,5,6,7-tetrahydro-4H-pyrrolo[3,2-c]pyridin-4-one trifluoroacetate).

This compound was prepared in 5% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: mp 206-210° C.; ¹H NMR (300 MHz, DMSO-d₆) δ 12.65 (br s, 1H), 8.59 (d, J=6.3 Hz, 1H), 8.50 (br s, 1H), 8.07 (d, J=16.4 Hz, 1H), 7.91 (br s, 1H), 7.63-7.58 (m, 2H), 7.39-7.10 (m, 6H), 3.65 (br s, 1H), 3.44-3.43 (m, 2H), 3.14-3.10 (m, 2H), 2.94 (t, J=6.7 Hz, 2H), 2.79-2.73 (m, 2H), 1.91-1.87 (m, 2H), 1.70-1.67 (m, 2H); ESI-MS m/z 415 [M+H]⁺.

### EXAMPLE 724

This example illustrates the preparation of 2-(2-{(E)-2-[4-morpholin-4-yl-2-(trifluoromethyl)phenyl]vinyl}pyridin-4-yl)-1,5,6,7-tetrahydro-4H-pyrrolo[3,2-c]pyridin-4-one trifluoroacetate.

Step 1: (Preparation of 4-morpholin-4-yl-2-(trifluoromethyl)benzaldehyde).

4-Morpholin-4-yl-2-trifluoromethyl-benzaldehyde was prepared in 99% yield by a procedure similar to the one described in step 1 of the synthesis of Example 707 using morpholine and 4-fluoro-2-trifluoromethyl-benzaldehyde: ¹H NMR (300 MHz, CDCl₃) δ 10.18 (d, J=1.7 Hz, 1H), 8.06 (d, J=8.9 Hz, 1H), 7.12 (d, J=2.4 Hz, 1H), 7.02 (dd, J=6.6, 2.3 Hz, 1H), 3.88 (t, J=5.0 Hz, 4H), 3.40 (t, J=5.0 Hz, 4H).

Step 2: (Preparation of vinyl boronate).

The above intermediate was prepared in 23% yield by a procedure similar to the one described in step 2 of the synthesis of Example 707 using 4-morpholin-4-yl-2-trifluoromethyl-benzaldehyde obtained in step 1.

Step 3: (Preparation of 2-(2-{(E)-2-[4-morpholin-4-yl-2-(trifluoromethyl)phenyl]vinyl}pyridin-4-yl)-1,5,6,7-tetrahydro-4H-pyrrolo[3,2-c]pyridin-4-one trifluoroacetate).

This compound was prepared in 6% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 221-224° C.; ¹H NMR (300 MHz, DMSO-d₆) δ 12.34 (s, 1H), 8.58 (d, J=6.0 Hz, 1H), 8.11 (s, 1H), 7.93 (d, J=16.3 Hz, 1H), 7.87 (s, 1H), 7.77 (d, J=4.48 Hz, 1H), 7.39 (s, 1H), 7.32 (d, J=9.3 Hz, 1H), 7.28-7.10 (m, 3H), 3.80-3.70 (m, 4H), 3.44 (t, J=6.3 Hz, 2H), 3.35-3.27 (m, 4H), 2.92 (t, J=7.5 Hz, 2H); ESI-MS m/z 469 [M+H]⁺.

### EXAMPLE 725

This example illustrates the preparation of 2-{2-[(E)-2-(2-methyl-4-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 4-fluoro-2-methylbenzaldehyde).

To a solution of 2-bomo-4-flourotoluene (7.85 g, 40 mmol) in anhydrous THF (150 mL) at −78° C., was added t-BuLi (47 mL, 80 mmol) drop wise. The dark brown solution was stirred at −78° C. for 1 h and at −50° C. for another hour. To the above solution anhydrous DMF (12.33 mL, 160 mmol) was added. The resulting solution was stirred for 1 h, warm up to room temperature and stirred over night. The reaction mixture was diluted with CH₂Cl₂ and water. The organic layer was isolated and washed with water, brine, dried (Na₂SO₄) and concentrated under reduced pressure. Purification by flash column chromatography (eluent 5:1 hexanes/Ether) gave 4-fluoro-2-methylbenzaldehyde (5.40 g, 97%) as a liquid; ¹H NMR (300 MHz, CDCl₃) δ 10.19 (s, 1H), 7.85-7.75 (m, 1H), 6.90-7.10 (m, 2H), 2.68 (s, 3H).

Step 2: (Preparation of 2-methyl-4-(morpholin-4-yl-benzaldehyde)):

This compound was prepared in 99% yield by a procedure similar to the one described in step 1 of the synthesis of Example 707 using morpholine and 4-fluoro-2-methylbenzaldehyde obtained in step 1.

Step 3: (Preparation of vinyl boronate intermediate).

The above intermediate was prepared in 48% yield by a procedure similar to the one described in step 2 of the synthesis of Example 707 using 2-methyl-4-mopholin-4-yl-benzaldehyde obtained in step 1.

Step 4: (Preparation of 2-{2-[(E)-2-(2-methyl-4-morpholin-4-ylphenyl)vinyl]pyridin-4-yl}-1,5,6,7-tetrahydro-4H-pyrrolo[3,2-c]pyridin-4-one trifluoroacetate). This compound was prepared in 16% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: mp 182-186° C.; ¹H NMR (300 MHz, DMSO-d₆) δ 12.47 (s, 1H), 8.55 (d, J=6.4 Hz, 1H), 8.32 (s, 1H), 8.05 (d, J=16.2 Hz, 1H), 7.81 (dd, J=4.9, 1.4 Hz, 1H), 7.62(d, J=8.8 Hz, 1H), 7.57 (d, J=1.9 Hz, 1H), 7.28 (s, 1H), 7.02 (d, J=16.2 Hz, 1H), 6.95-6.80 (m, 2H), 3.74 (t, J=5.1 Hz, 4H),3.47-3.42 (m, 2H), 3.42 (t, J=4.7 Hz, 4H), 2.93 (t, J=6.8 Hz, 2H), 2.48 (s, 3H); ESI-MS m/z 415 [M+H]⁺.

### EXAMPLE 726

This example illustrates the preparation of 2-(2-{(E)-2-[2-morpholin-4-yl-4-(morpholin-4-ylcarbonyl)phenyl]vinyl}pyridin-4-yl)-1,5,6,7-tetrahydro-4H-pyrrolo[3,2-c]pyridin-4-one trifluoroacetate.

Step 1: (Preparation of 2-(morpholin-4-yl)4-(morpholin-4-ylcarbonyl)benzaldehyde).

The above intermediate was obtained in 90% yield following a procedure similar to the one described in step 1 of the synthesis of Example 720 using morpholine and 4-carboxy-2-(morpholin-4-yl)benzaldehyde.

Step 2: (Preparation of vinyl boronate intermediate).

The above intermediate was prepared in 46% yield by a procedure similar to the one described in step 2 of the synthesis of Example 707 using the product obtained in step 1 above.

Step 3: (Preparation of 2-(2-{(E)-2-[2-morpholin-4-yl-4-(morpholin-4-ylcarbonyl) phenyl]vinyl}pyridin-4-yl)-1,5,6,7-tetrahydro-4H-pyrrolo[3,2-c]pyridin-4-one trifluoroacetate).

This compound was prepared in 10% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: mp 243-248° C.; ¹H NMR (300 MHz, CD₃OD) δ 8.46 (d, J=6.1 Hz, 1H), 8.18 (s, 1H), 8.09 (d, J=16.5 Hz, 1H), 7.79 (d, J=7.6 Hz, 2H), 7.45 (s, 1H), 7.32 (d, J=16.5 Hz, 1H), 7.28-7.21 (m, 2H), 3.93-3.86 (m, 4H), 3.80-3.40 (m, 10H), 3.10-2.98 (m, 6H); ESI-MS m/z 514 [M+H]⁺.

### EXAMPLE 727

This example illustrates the preparation of 2-(2-{(E)-2-[2-morpholin-4-yl-4-(pyrrolidin-1-ylcarbonyl)phenyl]vinyl}pyridin-4-yl)-1,5,6,7-tetrahydro-4H-pyrrolo[3,2-c]pyridin-4-one trifluoroacetate.

Step 1: (Preparation of 2-[morpholin-4-yl]-4-[pyrrolidin-1-ylcarbonyl]benzaldehyde).

The above intermediate was obtained in 90% yield following a procedure similar to the one described in step 1 of the synthesis of Example 720 using pyrrolidine and 4-carboxy-2-(morpholin-4-yl)benzaldehyde.

Step 2: (Preparation of vinyl boronate intermediate).

The above intermediate was prepared in 42% yield by a procedure similar to the one described in step 2 of the synthesis of Example 707 using the product obtained in step 1 above.

Step 3: (Preparation of 2-(2-{(E)-2-[2-morpholin-4-yl-4-(pyrrolidin-1-ylcarbonyl)phenyl]vinyl}pyridin-4-yl)-1,5,6,7-tetrahydro-4H-pyrrolo[3,2-c]pyridin-4-one trifluoroacetate).

This compound was prepared in 19% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 190-195° C.; ¹H NMR (300 MHz, DMSO-d₆) δ 12.38 (s, 1H), 8.61 (d, J=6.0 Hz, 1H), 8.29 (s, 1H), 8.02 (d, J=16.3 Hz, 1H), 7.88-7.78 (m, 1H), 7.69 (d, J=7.9 Hz, 1H), 7.49 (s, 1H), 7.38-7.18 (m, 4H), 3.82 (s, 4H), 3.53-3.25 (m, 6H), 3.10-2.83 (m, 6H), 2.11-1.72 (m, 4H); ESI-MS m/z 498 [M+H]⁺.

### EXAMPLE 728

This example illustrates the preparation of 2-{2-[(E)-2-(3-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 3-[morpholin-4-yl]-benzaldehyde).

A mixture of Pd₂ (dba)₃[tris (dibenzylidineacetone)dipalladium(0)] (114 mg, 0.125 mmol), 2-(di-tert-butyl-phosphino)biphenyl (74 mg, 025 mmol) and K₃PO₄ (7.42 g, 35 mmol) in toluene was degassed (4×, vacuum/argon). To that mixture was added 3-bromobenzaldehyde (4.62 g, 25 mmol) and morpholine (2.61 g, 30 mmol). The resulting mixture was degassed (4×, vacuum/argon) and heated at 100° C. over night. The cooled reaction mixture was diluted with CH₂Cl₂ (200 mL) and water (100 mL). The aqueous layer was extracted with additional amount of CH₂Cl₂ (2×100 mL). The combined organic layer was washed with brine, dried (Na₂SO₄) and concentrated under reduced pressure. Purification by flashed column chromatography (eluent 4:1 hexanes/EtOAc) gave 3-morpholin-4-yl-benzaldehyde as an oil (0.95 g, 20%); ¹H NMR (300 MHz, CDCl₃) δ 9.97 (s, 1H), 7.50-7.30 (m, 3H), 7.20-7.15 (m, 1H), 3.88 (t, J=4.9 Hz, 4H), 3.32 (t, J=4.9 Hz, 4H).

Step 2: (Preparation of vinyl boronate intermediate).

The above intermediate was prepared in 43% yield by a procedure similar to the one described in step 2 of the synthesis of Example 707 using 3-[morpholin-4-yl]-benzaldehyde obtained in step 1.

Step 3: (Preparation of 2-{2-[(E)-2-(3-morpholin-4-ylphenyl)vinyl]pyridin-4-yl}-1,5,6,7-tetrahydro-4H-pyrrolo[3,2-c]pyridin-4-one trifluoroacetate).

This compound was prepared in 5% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 198-201° C.; ¹H NMR (300 MHz, DMSO-d₆) δ 12.39 (s, 1H), 8.61 (d, J=6.2 Hz, 1H), 8.36 (s, 1H), 7.90 (d, J=16.4 Hz, 1H), 7.84 (d, J=5.6 Hz, 1H), 7.54 (s, 1H), 7.38-7.05 (m, 6H), 3.79-3.76 (m, 4H), 3.47-3.43 (m, 2H), 3.18 (t, J=4.5 Hz, 4H), 2.92 (t, J=6.8 Hz, 2H); ESI-MS m/z 401[M+H]⁺.

### EXAMPLE 729

This example illustrates the preparation of 2-(2-{(E)-2-[4-(dimethylamino)-2,6-difluorophenyl]vinyl}pyridin-4-yl)-1,5,6,7-tetrahydro-4H-pyrrolo[3,2-c]pyridin-4-one trifluoroacetate.

Step 1: (Preparation of N,N-dimethyl-3,5-difluoroaniline).

A mixture of 3,5-difluoroaniline (5.0 g, 38.7 mmol) and trimethyl phosphate (3.0 mL, 25.8 mmol) was heated under reflux for 2 h. The reaction mixture was cooled to 50° C., and a solution of NaOH (3.2 g) in water (12 mL) was added to it. The reaction mixture was heated to 100° C. for 1 h, and then cooled to room temperature. The reaction mixture was diluted with water (100 mL), and the product was extracted into Et₂O (3×100 mL). The organic extract was filtered through a plug of basic alumina, and the filtrate was concentrated to give N,N-dimethyl-3,5-difluoroaniline (5.2 g, 85%) as a red oil, which was used in step 2 without further purification. ¹H NMR δ (300 MHz, CDCl₃) δ 6.16-6.12 (m, 3H), 2.94 (s, 6H).

Step 2: (Preparation of 4-dimethlyamino-2,6-difluorobenzaldehyde).

To a solution of N,N-dimethyl-3,5-difluoroaniline (2.0 g, 12.7 mmol) in THF (45 mL) at −78° C. was added n-BuLi (5.6 mL, 14 mmol, 2.5 M solution in hexanes) dropwise. After 30 min, DMF (1.5 mL, 19 mmol) was added to the reaction mixture, which was then warmed to room temperature slowly. The reaction mixture was poured on ice, and the product was extracted into Et₂O (3×75 mL). The organic extract was washed with brine, and concentrated under reduced pressure. Purification by flash chromatography (eluent 90:10 to 60:40 hexanes/EtOAc) gave 4-dimethlyamino-2,6-difluorobenzaldehyde (1.51 g, 64%) as an off-white solid: ¹H NMR (300 MHz, CDCl₃) δ 10.06 (s, 1H), 6.13 (d, J=13.0 Hz, 2H), 3.07 (s, 6H).

Step 3: (Preparation of vinyl boronate intermediate).

The above intermediate was prepared in 63% yield by a procedure similar to the one described in step 2 of the synthesis in Example 707 using 4-dimethlyamino-2,6-difluorobenzaldehyde obtained in step 2.

Step 4: (Preparation of 2-(2-{(E)-2-[4-(dimethylamino)-2,6-difluorophenyl]vinyl}pyridin-4-yl)-1,5,6,7-tetrahydro-4H-pyrrolo[3,2-c]pyridin-4-one trifluoroacetate):

This compound was prepared in 2% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: mp 189-193° C.; ¹H NMR (300 MHz, DMSO-d₆) δ 12.28 (br s, 1H), 8.52 (d, J=6.2 Hz, 1H), 8.28 (br s, 1H), 7.82-7.76 (m, 2H), 7.54 (br s, 1H), 7.25 (br s, 1H), 7.18 (d, J=16.5 Hz, 1H), 6.54 (d, J=13.5 Hz, 2H), 3.24 (t, J=6.6 Hz, 2H), 3.14 (s, 6H), 2.92 (t, J=6.6 Hz, 2H); ESI-MS m/z 395 [M+H]⁺.

### EXAMPLE 730

This example illustrates the preparation of 2-(2-{(E)-2-[2-morpholin-4-yl-4-(trifluoromethyl)phenyl]vinyl}pyridin-4-yl)-1,5,6,7-tetrahydro-4H-pyrrolo[3,2-c]pyridin-4-one trifluoroacetate.

Step 1: (Preparation of 2-(4-morpholino)-3-(trifluoromethyl)benzaldehyde).

The above compound was prepared in 81% yield by a procedure similar to the one described in step 1 in the synthesis of Example 707 using 2-fluoro-3-(trifluoromethyl)benzaldehyde and morpholine: ¹H NMR (300 MHz, CDCl₃) δ 10.34 (s, 1H), 7.91 (d, J=8.1 Hz, 1H), 7.39 (d, J=7.8 Hz, 1H), 7.34 (s, 1H), 3.92 (t, J=4.5 Hz, 4H), 3.14 (t, J=4.5 Hz, 4H).

Step 2: (Preparation of vinyl boronate intermediate).

The above intermediate was prepared in 16% yield by a procedure similar to the one described in step 2 of the synthesis of Example 707 the 2-(4-morpholino)-3-(trifluoromethyl)benzaldehyde obtained in step 1.

Step 3: (Preparation of 2-(2-7(E)-2-[2-morpholin-4-yl-4-(trifluoromethyl)phenyl]vinyl}pyridin-4-yl)-1,5,6,7-tetrahydro-4H-pyrrolo[3,2-c]pyridin-4-one trifluoroacetate ).

This compound was prepared in 63% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 155-160° C.; ¹H NMR (300 MHz, DMSO-d₆) δ 12.39 (s, 1H), 8.63 (d, J=6.1 Hz, 1H), 8.32 (s, 1H), 8.02 (d, J=16.5 Hz, 1H), 7.87-7.85 (m, 2H), 7.52-7.50 (m, 2H), 7.39-7.27 (s, 3H), 3.73 (s, 4H), 3.47-3.37 (m, 2H), 3.01 (s, 4H), 2.93 (t, J=6.7 Hz, 2H); ESI-MS m/z 469 [M+H]⁺.

### EXAMPLE 731

This example illustrates the preparation of 2-(2-{(E)-2-[2-(morpholin-4-ylcarbonyl)phenyl]vinyl}pyridin-4-yl)-1,5,6,7-tetrahydro-4H-pyrrolo[3,2-c]pyridin-4-one trifluoroacetate.

Step 1: (Preparation of 2-(morpholin-4-ylcarbonyl)benzaldehyde).

The above intermediate was obtained in 90% yield following a procedure similar to the one described in step 1 of the synthesis of Example 720 using morpholine and 2-carboxybenzaldehyde.

Step 2: (Preparation of vinyl boronate intermediate).

The above intermediate was prepared in 45% yield by a procedure similar to the one described in step 2 of the synthesis of Example 707 using the 2-(morpholin-4-ylcarbonyl)benzaldehyde obtained in step 1.

Step 3: (Preparation of 2-(2-{(E)-2-[2-(morpholin-4-ylcarbonyl)phenyl]vinyl}pyridin-4-yl)-1,5,6,7-tetrahydro-4H-pyrrolo[3,2-c]pyridin-4-one trifluoroacetate).

This compound was prepared in 1% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 174-178° C.; ¹H NMR (300 MHz, DMSO-d₆) δ 12.37 (s, 1H), 8.60 (d, J=5.9 Hz, 1H), 8.19 (s, 1H), 7.87-7.73 (m, 3H), 7.60-7.32 (m, 4H), 7.28-7.17 (m, 2H), 3.69 (s, 4H), 3.50-3.35 (m, 4H), 3.20-2.98 (m, 2H), 2.91 (d, J=6.7 Hz, 2H); ESI-MS m/z 429 [M+H]⁺.

### EXAMPLE 732

This example illustrates the preparation of N,N-dimethyl-3-morpholin-4-yl-4-{(E)-2-[4-(4-oxo-4,5,6,7-tetrahydro-1H-pyrrolo[3,2-c]pyridin-2-yl)pyridin-2-yl]vinyl}benzamide trifluoroacetate.

Step 1: (Preparation of 2-[4-bromo-2-(4-morpholino)phenyl]-1,3-dioxolane).

To a solution of 4-bromo-2-(4-morpholino)benzaldehyde (15.0 g, 55.5 mmol) obtained in step 1 of the synthesis of Example 714 in benzene (300 mL) was added pTsOH (5.28 g, 27.8 mmol) and ethylene glycol (15.5 mL, 277.9 mmol). The mixture was heated to reflux for 30 min, while removing water through a Dean-Stark apparatus. The solution was concentrated to a smaller volume, diluted with CHCl₃ (500 mL) and basified with a saturated solution of NaHCO₃ till pH˜9. The aqueous phase was extracted with additional amount of CHCl₃. The combined organic phase was dried and concentrated under reduced pressure. The desired acetal (10.3 g, 59%) was obtained as a tan solid by recrystallizing the crude product with a mixture of hexanes and EtOAc: ¹H NMR (300 MHz, CDCl₃) 67.44 (d, J=7.4 Hz, 1H), 7.30-7.18 (m, 2H), 6.12 (s, 1H), 4.21-4.12 (m, 2H), 4.06-3.97 (m, 2H), 3.87-3.78 (m, 4H), 3.00 (t, J=4.5 Hz, 4H).

Step 2: (Preparation of 4-formyl-3-(4-morpholino)benzoic acid).

To a solution of the acetal (10.0 g, 31.82 mmol) obtained in step 1 in THF (146 mL) at −78° C. was added drop wise a solution of t-BuLi (37.4 mL, 63.6 mmol, 1.7 M in pentane). The resulting mixture was stirred for 30 min and dry CO₂ gas was bubbled into the reaction mixture for 50 min. The reaction mixture was warmed to 0° C. and stirred for an additional 40 min under CO₂ atmosphere. The reaction mixture was acidified with concentrated HCl (50 mL) and extracted with additional amount of CH₂Cl₂ (3×300 mL). The combined organic layer was concentrated to dryness under reduced pressure and the residue was triturated with MeOH to afford a first crop of the desired product (4.64 g, 62%) as an orange solid. Another extraction of the aqueous phase gave a second crop (1.1 g, 15%): mp 226-229° C.; ¹H NMR (300 MHz, DMSO-d₆) δ 13.40 (s, 1H), 10.26 (s, 1H), 7.80 (d, J=8.3 Hz, 1H), 7.69 (s, 1H), 7.67 (d, J=8.3 Hz, 1H), 8.30-7.10 (m, 4H), 3.11-2.98 (m, 4H).

Step 3: (Preparation of 4-(N,N-dimethylaminocarbonyl)-2-(4-morpholino)benzaldehyde).

To a solution of 4-formyl-3-(4-morpholino)benzoic acid (0.8 g, 3.4 mmol) obtained in step 2 in DMF (14 mL) was added N,N-dimethylamine (3.4 mL, 2M in THF, 6.8 mmol), EDCl (1.3 g, 6.8 mmol) and HOBt (0.68 g, 5.1 mmol). The mixture was stirred overnight in a sealed tube and was diluted with CH₂Cl₂ (100 mL) and H₂O (20 mL). The aqueous phase was extracted with additional amount of CH₂Cl₂ (3×60 mL). The combined organic phase was dried (Na₂SO₄) and concentrated under reduced pressure. Purification by flash chromatography (eluent 98:2 to 96:4 CH₂Cl₂/MeOH) gave 4-(N,N-dimethylaminocarbonyl)-2-(4-morpholino)benzaldehyde (0.85 g, 96%). ¹H NMR (300 MHz, CDCl₃) δ 10.30 (s, 1H), 7.83 (d, J=7.7 Hz, 1H), 7.17-7.10 (m, 2H), 3.95-3.87 (m, 4H), 3.17-3.08 (m, 7H), 2.96 (s, 3H).

Step 4: (Preparation of vinyl boronate intermediate).

The above intermediate was prepared in 70% yield by a procedure similar to the one described in step 2 of the synthesis of Example 707 using the 4-(N,N-dimethylaminocarbonyl)-2-(4-morpholino)benzaldehyde obtained in step 3.

Step 5: (Preparation of N,N-dimethyl-3-morpholin-4-yl-4-{(E)-2-[4-(4-oxo-4,5,6,7-tetrahydro-1H-pyrrolo[3,2-c]pyridin-2-yl)pyridin-2-yl]vinyl}benzamide trifluoroacetate).

This compound was prepared in 4% yield by the cross coupling of the vinylboronate from step 4 above and 2-(2-Chloropyridin-4-yl)-1,5,6,7-tetrahydro-4H-pyrrolo[3,2-c]pyridin-4-one according to the general procedure described for Example 551: mp 155-160° C.; ¹H NMR (300 MHz, DMSO-d₆) δ 12.39 (s, 1H), 8.60 (d, J=6.0 Hz, 1H), 8.28 (s, 1H), 8.03 (d, J=16.4 Hz, 1H), 7.87-7.77 (m, 1H), 7.66 (d, J=7.9 Hz, 1H), 7.47 (s, 1H), 7.31 (d, J=16.4 Hz, 1H), 7.25 (s, 1H), 7.22-7.08 (m, 2H), 3.82 (s, 4H), 3.46-3.30 (m, 2H), 2.98-2.88 (m, 12H); ESI-MS m/z 472 [M+H]⁺.

### EXAMPLE 733

This example illustrates the preparation of 2-(2-{(E)-2-[2-(2-morpholin-4-ylmethyl)phenyl]vinyl}pyridin-4-yl)-1,5,6,7-tetrahydro-4H-pyrrolo[3,2-c]pyridin-4-one trifluoroacetate.

Step 1: (Preparation of 4-(2-bromophenylmethyl)morpholine).

To an ice-cold solution of morpholine (1.92 mL, 22.0 mmol) in DMF (80 mL) was added 60% NaH (2.66 g, 40.0 mmol) and the mixture was stirred for 45 min prior to the addition of Nal (1.5 g, 10.0 mmol) and 2-bromobenzylbromide (5.0 g, 20 mmol). The solution thus obtained was stirred for 1.5 h while warming to room temperature. Then, the reaction mixture was diluted with Et₂O (300 mL) and H₂O (20 mL) was added dropwise. The aqueous layer was extracted with additional amount of Et₂O (3×60 mL). The combined organic layer was washed with H₂O (3×15 mL), brine (2×15 mL), dried and concentrated under reduced pressure to give 4-(2-bromophenylmethyl)morpholine.: ¹H NMR (300 MHz, CDCl₃) δ 7.54 (dd, J=7.7, 1.1 Hz, 1H), 7.46 (dd, J=7.6, 1.6 Hz, 1H), 7.28 (td, J=7.4, 1.1 Hz, 1H), 7.10 (td, J=7.6, 1.7 Hz, 1H), 3.74-3.64 (m, 4H), 3.59 (s, 2H), 2.52 (t, J=4.6 Hz, 4H).

Step 2: (Preparation of 2-(4-morpholinomethyl)benzaldehyde).

To a solution at of 4-(2-bromo-benzyl)-morpholine (20 mmol) obtained in step 1 in THF (92 mL) -78° C. was added t-BuLi (23.5 mL, 39.9 mmol) and the solution was stirred for 30 min. To that mixture was added DMF (3.11 mL, 40 mmol) and the solution thus obtained was stirred for 1 h while warming to room temperature. The reaction was quenched by adding a saturated aqueous solution of NH₄Cl (18 mL) and the aqueous phase was extracted with CH₂Cl₂ (3×60 mL). The combined organic phase was dried (Na₂SO₄) and concentrated under reduced pressure. Purification by flash chromatography (eluent 1:1:1 to 1:1:2 to 1.5:0.5:2 CH₂Cl₂/hexanes/EtOAc) gave the desired 2-(4-morpholinomethyl)benzaldehyde (3.81 g, 93% over 2 steps): ¹H NMR (300 MHz, CDCl₃) δ 10.43 (s, 1H), 7.88 (d, J=7.6 Hz, 1H), 7.55-7.23 (m, 3H), 3.81 (s, 2H), 3.65 (t, J=4.5 Hz, 4H), 2.46 (t, J=4.4 Hz, 4H).

Step 3: (Preparation of vinyl boronate intermediate).

The above intermediate was prepared in 46% yield by a procedure similar to the one described in step 2 of the synthesis of EXAMPLE 707 using the 2-(4-morpholinomethyl)benzaldehyde obtained in step 2.

Step 4: (Preparation of 2-(2-{(E)-2-[2-(2-morpholin-4-ylmethyl)phenyl]vinyl}pyridin-4-yl)-1,5,6,7-tetrahydro-4H-pyrrolo[3,2-c]pyridin-4-one trifluoroacetate).

This compound was prepared in 39% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: mp 115-120° C.; ¹H NMR (300 MHz, DMSO-d₆) δ 12.48 (s, 1H), 8.70 (d, J=6.1 Hz, 1H), 8.38 (s, 1H), 8.16 (d, J=16.0 Hz, 1H), 7.91-7.85 (m, 2H), 7.70-7.48 (m, 4H), 7.35-7.23 (m, 2H), 4.64 (s, 2H), 4.09-3.55 (m, 4H), 3.47-3.42 (m, 2H), 3.25 (s, 4H), 2.92 (t, J=6.7 Hz, 2H); ESI-MS m/z 415 [M+H]⁺.

### EXAMPLE 734

This example illustrates the preparation of 2-(2-{(E)-2-[2-(2-morpholin-4-ylethyl)phenyl]vinyl}pyridin-4-yl)-1,5,6,7-tetrahydro-4H-pyrrolo[3,2-c]pyridin-4-one trifluoroacetate.

Step 1: (Preparation of 4-(2-bromophenylacetyl)morpholine).

The above intermediate was prepared in a quantitative yield following a procedure similar to the one described in step 1 for the synthesis of Example 720 using morpholine and 2-bromophenylacetic acid: ¹H NMR (300 MHz, CDCl₃) δ 7.55 (d, J=7.5 Hz, 1H), 7.32-7.24 (m, 2H), 7.18-7.06 (m, 1H), 3.82 (s, 2H), 3.67 (s, 4H), 3.65-3.57 (m, 2H), 3.51-3.44 (m, 2H).

Step 2: (Preparation of 4-[2(2-bromophenyl)ethyl]morpholine).

To a solution of 4-(2-bromophenylacetyl)morpholine obtained in step 1 in THF (53 mL) was added a solution of BH₃-THF (53 mL, 53 mmol, 1 M in THF) and the reaction was heated to reflux for 1.5 h. To the cooled reaction mixture was added MeOH (20 mL) drop wise and the resulting mixture was stirred at room temperature for 30 min, then, concentrated under reduced pressure. The residue was dissolved in MeOH (50 mL) and treated with a solution of 2 N HCl (50 mL). The resulting mixture was heated to reflux for 3 h, concentrated to a smaller volume under reduced pressure, then, diluted with a solution of 2 N NaOH (100 mL). The aqueous phase was extracted with CH₂Cl₂ (3×150 mL) and the combined organic phase was dried (Na₂SO₄) and concentrated to give the desired product: ¹H NMR (300 MHz, CDCl₃) δ 7.51 (d, J=8.1 Hz, 1H), 7.26-7.18 (m, 2H), 7.12-7.03 (m, 1H), 3.74 (t, J=4. Hz, 4H), 3.00-2.88 (m, 2H), 2.63-2.48 (m, 6H).

Step 3: (Preparation of 2-[2-(4-morpholino)ethyl]benzaldehyde).

The formylation of the intermediate obtained in step 2 reaction was achieved in quantitative yield following a procedure similar to the one used in step 2 of the synthesis of Example 733. Purification by flash chromatography (eluent 96:4 CH₂Cl₂/MeOH) gave the above aldehyde.

Step 4: (Preparation of vinyl boronate intermediate).

The above intermediate was prepared in 40% yield by a procedure similar to the one described in step 2 of the synthesis of Example 707 using the 2-(4-morpholinoeethyl)benzaldehyde obtained in step 3.

Step 5: (Preparation of 2-(2-{(E)-2-[2-(2-morpholin-4-ylethyl)phenyl]vinyl}pyridin-4-yl)-1,5,6,7-tetrahydro-4H-pyrrolo[3,2-c]pyridin-4-one trifluoroacetate).

This compound was prepared in 32% yield by the cross coupling of the vinylboronate from step 4 above and 2-(2-Chloropyridin-4-yl)-1,5,6,7-tetrahydro-4H-pyrrolo[3,2-c]pyridin-4-one according to the general procedure described for Example 551: mp 220-223° C.; ¹H NMR (300 MHz, CD₃OD) δ 8.50 (d, J=6.3 Hz, 1H), 8.29-8.21 (m, 1H), 8.13 (d, J=16.1 Hz, 1H), 7.88-7.76 (m, 2H), 7.48-7.35 (m, 4H), 7.26 (d, J=16.1 Hz, 1H), 4.00-3.90 (m, 4H), 3.61 (t, J=6.9 Hz, 2H), 3.48-3.40 (m, 4H), 3.36 (s, 4H), 3.02 (t, J=6.9 Hz, 2H); ESI-MS m/z 429 [M+H]⁺.

### EXAMPLE 735

This example illustrates the preparation of 2-(2-{(E)-2-[3-(morpholin-4-ylcarbonyl)phenyl]vinyl}pyridin-4-yl)-1,5,6,7-tetrahydro-4H-pyrrolo[3,2-c]pyridin-4-one trifluoroacetate.

Step 1: (Preparation of 4-(morpholin-4-carbonyl)-benzaldehyde).

4-(Morpholin-4-carbonyl)-benzaldehyde was prepared in 70% yield by a procedure similar to the one described in step 1 of the synthesis of Example 712 using morpholine and 3-carboxybenzaldehyde. ¹H NMR (300 MHz, CDCl₃) δ 10.05 (s, 1H), 8.00-7.90 (m, 2H), 7.63-7.55 (m, 2H), 3.90-3.30 (m, 8H).

Step 2: (Preparation of vinyl boronate intermediate).

The above intermediate was prepared in 28% yield by a procedure similar to the one described in step 2 of the synthesis of Example 707 using the 2-(4-morpholinoeethyl)benzaldehyde obtained in step 1.

Step 3: (Preparation of 2-(2-{(E)-2-[3-(morpholin-4-ylcarbonyl)phenyl]vinyl}pyridin-4-yl)-1,5,6,7-tetrahydro-4H-pyrrolo[3,2-c]pyridin-4-one trifluoroacetate).

This compound was prepared in 8% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: mp 204-207° C.; ¹H NMR (300 MHz, DMSO-d₆) δ 12.34 (s, 1H), 8.61 (d, J=6.0 Hz, 1H), 8.29 (s, 1H), 7.93 (d, J=16.3 Hz, 1H), 7.85-7.65 (m, 3H), 7.57 (t, J=7.7 Hz, 1H), 7.50-7.40 (m, 2H), 7.34 (d, J=16.4 Hz, 1H), 7.24 (s, 1H), 3.80-3.30 (m, 10H), 2.92 (t, J=6.7 Hz, 2H); ESI-MS m/z 429 [M+H]⁺.

### EXAMPLE 736

This example illustrates the preparation of 2-[2-((E)-2-{4-[(2R,6S)-2,6-dimethylmorpholin-4-yl]phenyl}vinyl)pyridin-4-yl]-1,5,6,7-tetrahydro-4H-pyrrolo[3,2-c]pyridin-4-one trifluoroacetate.

Step 1: (Preparation of 4-(2,6-dimethyl-morpholin-4-yl)-benzaldehyde).

4-(2,6-Dimethyl-morpholin-4-yl)-benzaldehyde was prepared in 66% yield by a procedure similar to the one described in step 1 of the synthesis of Example 707 using 2,6-dimethylmorpholine and 4-fluorobenzaldehyde; ¹H NMR (300 MHz, CDCl₃) δ 9.79 (s, 1H), 7.80-7.70 (m, 2H), 6.91 (d, J=8.9 Hz, 2H), 3.55-3.85 (m, 4H), 2.60-2.50 (m, 2H), 1.30-1.20 (m, 6H).

Step 2: (Preparation of vinyl boronate intermediate).

The above intermediate was prepared in 78% yield by a procedure similar to the one described in step 2 of the synthesis of Example 707 using the 4-(2,6-dimethyl-morpholin-4-yl)-benzaldehyde obtained in step 1.

Step 3: (Preparation of 2-[2-((E)-2-{4-[(2R,6S)-2,6-dimethylmorpholin-4-yl]phenyl}vinyl)pyridin-4-yl]-1,5,6,7-tetrahydro-4H-pyrrolo[3,2-c]pyridin-4-one trifluoroacetate).

This compound was prepared in 21% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 173-177° C.; ¹H NMR (300 MHz, DMSO-d₆) δ 12.43 (s, 1H), 8.53 (d, J=6.4 Hz, 1H), 8.36 (s, 1H), 7.89 (d, J=16.3 Hz, 1H), 7.80 (d, J=6.4 Hz, 1H), 7.60-7.50 (m, 3H), 7.27 (s, 1H), 7.15-7.00 (m, 3H), 3.85-3.3.60 (m, 4H), 3.55-3.40 (m, 2H), 2.92 (t, J=6.6 Hz, 2H), 2.37 (t, J=11.3 Hz, 2H), 1.25-1.05 (m, 6H); ESI-MS m/z 429 [M+H]⁺.

### EXAMPLE 737

This example illustrates the preparation of 2-[2-((E)-2-{2-[(2R,6S)-2,6-dimethylmorpholin-4-yl]phenyl}vinyl)pyridin-4-yl]-1,5,6,7-tetrahydro-4H-pyrrolo[3,2-c]pyridin-4-one trifluoroacetate.

Step 1: (Preparation of 2-(cis-2,6-dimethylmorpholin-4-yl)benzaldehyde).

2-(cis-2,6-dimethylmorpholin-4-yl)benzaldehyde was prepared in 84% yield by a procedure similar to the one described in step 1 of the synthesis of Example 707 using 2-fluorobenzaldehye and cis-2,6-dimethylmorpholine. ¹H NMR (300 MHz, CDCl₃) δ 10.32 (s, 1H), 7.81 (dd, J=7.7, 1.5 Hz, 1H), 7.54-7.51 (m, 1H), 7.16-7.08 (m, 2H), 3.95-3.89 (m, 2H), 3.08 (d, J=11.3 Hz, 2H), 2.65 (t, J=11.4 Hz, 2H), 1.23 (d, J=6.3 Hz, 6H).

Step 2: (Preparation of vinyl boronate intermediate).

The above intermediate was prepared in 38% yield by a procedure similar to the one described in step 2 of the synthesis of Example 707 using the 2-(cis-2,6-dimethylmorpholin-4-yl)benzaldehyde obtained in step 1.

Step 3: (Preparation of 2-[2-((E)-2-{2-[(2R,6S)-2,6-dimethylmorpholin-4-yl]phenyl}vinyl)pyridin-4-yl]-1,5,6,7-tetrahydro-4H-pyrrolo[3,2-c]pyridin-4-one trifluoroacetate).

This compound was prepared in 10% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 175-178° C.; ¹H NMR (300 MHz, DMSO-d₆) δ 12.38 (br s, 1H), 8.59 (d, J=6.2 Hz, 1H), 8.28 (br s, 1H), 8.04 (d, J=16.4 Hz, 1H), 7.90-7.80 (m, 1H), 7.65 (d, J=7.7 Hz, 1H), 7.49 (br s, 1H), 7.41-7.14 (m, 5H), 3.92-3.90 (m, 2H), 3.45-3.43 (m, 2H), 3.03 (d, J=11.0 Hz, 2H), 2.93 (t, J=6.8 Hz, 2H), 2.51-2.40 (m, 2H), 1.10 (d, J=6.2 Hz, 6H); ESI-MS m/z 429 [M+H]⁺.

### EXAMPLE 738

This example illustrates the preparation of 2-{2-[(E)-2-(4-piperazin-1-ylphenyl)vinyl]pyridin-4-yl}-1,5,6,7-tetrahydro-4H-pyrrolo[3,2-c]pyridin-4-one trifluoroacetate.

Step 1: (Preparation of 4-(piperazin-1-yl)benzaldehyde).

A mixture of 4-fluorobenzadehyde (8.27 g, 66 mmol), piperazine (17.2 g, 199 mmol) and K₂CO₃ (18.20 g, 132 mmol) in DMF (75 mL) was heated overnight at 153° C. The cooled reaction mixture was diluted with CH₂Cl₂ (400 mL), and water (100 mL). The organic layer was separated, washed with brine, dried (Na₂SO₄) and concentrated under reduced pressure. The crude mixture was used in the next step with out purification.

Step 2: (Preparation of 1,1-dimethylethyl 4-(4-formylphenyl)-1-piperazine-carboxylate).

To a stirred solution of 4-(piperazin-1-yl)benzaldehyde (3.80 g, 19.97 mmol) obtained in step 1 in CH₂Cl₂ (35 mL), was added Boc₂O (6.35 g, 29.96 mmol) and DMAP (224 mg, 1.99 mmol). The resulting solution was stirred at room temperature for 2 h. The reaction mixture was diluted with CH₂Cl₂ (100 mL) and water (100 mL). The organic phase was separated and the aqueous layer was extracted with more CH₂Cl₂ (2×50 mL). The combined organic phase was washed with brine, dried (Na₂SO₄) and concentrated under reduced pressure. Purification by flash column chromatography (eluent 3:2 hexanes/EtOAc) gave 1,1-dimethylethyl 4-(4-formylphenyl)-1-piperazinecarboxylate (4.96 g, 61%, over 2 steps) as an off-white solid. ¹H NMR (300 MHz, CDCl₃) 69.83 (s, 1H), 7.80 (dd, J=5.1, 2.0 Hz, 2H), 6.93 (d, J=8.9 Hz, 2H), 3.62 (t, J=5.0 Hz, 4H), 3.42 (t, J=5.5 Hz, 4H), 1.52 (s, 9H).

Step 3: (Preparation of vinyl boronate intermediate).

The above intermediate was prepared in 85% yield by a procedure similar to the one described in step 2 of the synthesis of Example 707 using the 1,1-dimethylethyl 4-(4-formylphenyl)-1-piperazinecarboxylate obtained in step 2.

Step 4: (Preparation of 2-{2-[(E)-2-(4-piperazin-1-ylphenyl)vinyl]pyridin-4-yl}-1,5,6,7-tetrahydro-4H-pyrrolo[3,2-c]pyridin-4-one trifluoroacetate).

This compound was prepared in 2% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: mp 210-220° C.; ¹H NMR (300 MHz, DMSO-d₆) δ 12.37 (s, 1H), 8.82 (s, 1H), 8.55 (d, J=6.2 Hz, 1H), 8.98-8.78 (m, 2H), 7.86 (d, J=16.2 Hz, 1H), 7.75 (d, J=5.6 Hz, 2H), 7.58 (d, J=8.8 Hz, 1H), 7.48 (s, 1H), 7.24 (s, 1H), 7.15-7.05 (m, 3H), 3.55-3.35 (m, 6H), 3.30-3.15 (m, 4H), 2.92 (t, J=6.8 Hz, 2H); ESI-MS m/z 400 [M+H]⁺.

### EXAMPLE 739

This example illustrates the preparation of 2-{2-[(E)-2-(2,6-difluoro-4-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 4-(3,5-difluorophenyl)morpholine).

Pd₂(dba)₃ (0.12 g, 0.13 mmol), 2-(di-tert-butyl-phosphino)biphenyl (77 mg, 0.26 mmol) and K₃PO₄ (7.7 g, 36.3 mmol) were placed in an oven-dried round bottom flask. The flask was degassed (3×, vacuum/argon), and toluene (52 mL) was added to it. The reaction mixture was degassed again (3×, vacuum/argon), and 1-bromo-3,5-difluorobenzene (3 mL, 25.9 mmol) and morpholine (2.7 mL, 31.1 mmol) were added to it. The reaction mixture was heated at 100° C. for 5 h. An additional amount of both catalyst (60 mg, 0.06 mmol) and ligand (35 mg, 0.12 mmol) were added to the reaction mixture, which was then heated at 100° C. overnight. The cooled reaction mixture was partitioned between CH₂Cl₂ (150 mL) and water (100 mL), and the organic layer was washed with brine and concentrated under reduced pressure. Purification by flash chromatography (eluent 90:10 to 70:30 hexanes/EtOAc) gave 4-(3,5-difluorophenyl)morpholine (1.88 g, 36%) as yellow needles: ¹H NMR (300 MHz, CDCl₃) δ 6.38-6.29 (m, 3H), 3.84 (t, J=4.9 Hz, 4H), 3.15 (t, J=4.9 Hz, 4H).

Step 2: (Preparation of 2,6-difluoro-4-(morpholin-4-yl)benzaldehyde).

To a solution of 4-(3,5-difluorophenyl)morpholine (1.03 g, 5.2 mmol) in THF (35 mL) at −78° C. was added TMEDA (1.3 mL, 6.47 mmol), followed by n-BuLi (2.6 mL, 6.47 mmol, 2.5 M solution in hexanes) dropwise. After 30 min, DMF (0.8 mL, 10.4 mmol) was added to the reaction mixture, which was warmed to room temperature overnight. The mixture was poured into water (100 mL), and the product was extracted into Et₂O (3×50 mL). The combined organic extract was washed with brine and concentrated under reduce pressure. Purification by flash chromatography (eluent 95:5 to 70:30 hexanes/EtOAc) gave 2,6-difluoro-4-morpholin-4-yl-benzaldehyde (0.81 g, 66%) as an off-white solid: ¹H NMR (300 MHz, CDCl₃) δ 10.10 (s, 1H), 6.32 (d, J=12.8 Hz, 2H), 3.84 (t, J=5.0 Hz, 4H), 3.33 (t, J=5.0 Hz, 4H).

Step 3: (Preparation of 4-[3,5-difluoro-4-(2,2-dibromoethenyl)phenyl]morpholine).

To a mixture of 2,6-difluoro-4-morpholin-4-yl-benzaldehyde (1.4 g, 6.2 mmol) and CBr₄ (2.14 g, 6.47 mmol) was added CH₂Cl₂ (18.5 mL), and the resultant pale yellow solution was cooled in an ice-bath. Triphenylphosphine (3.40 g, 12.95 mmol) was added to the mixture in 4 portions, and the ice-bath was removed. After 1 h at room temperature, the reaction mixture was concentrated under reduced pressure. Purification by flash chromatography (eluent 95:5 to 60:40 hexanes/EtOAc) gave 4-[3,5-difluoro-4-(2,2-dibromoethenyl)phenyl]morpholine (1.08 g, 45%) as pale yellow crystals: ¹H NMR (300 MHz, CDCl₃) 87.19 (s, 1H), 6.41-6.36 (m, 2H), 3.84 (t, J=4.9 Hz, 4H), 3.18 (t, J=4.9 Hz, 4H).

Step 4: (Preparation of 4-[3,5-difluoro-4-ethynylphenyl]morpholine).

To a solution of n-BuLi (2.5 mL, 6.2 mmol, 2.5 M solution in hexanes) in THF (5 mL) at −78° C. was added a solution of 4-[3,5-difluoro-4-(2,2-dibromoethenyl)phenyl]morpholine (1.08 g, 2.82 mmol) from step 3 above in THF (5 mL) dropwise. After 1 h at −78° C., saturated NH₄Cl (7 mL) was added to the reaction mixture, which was then warmed to room temperature. The reaction mixture was partitioned between water (75 mL) and CH₂Cl₂ (150 mL), and the organic layer was washed with brine and concentrated under reduced pressure. Purification by flash chromatography (eluent 95:5 to 60:40 hexanes/EtOAc) gave 4-[3,5-difluoro-4-ethynylphenyl]morpholine (0.51 g, 81%) as a white solid: ¹H NMR (300 MHz, CDCl₃) δ 6.37 (d, J=10.9 Hz, 2H), 3.83 (t, J=4.9 Hz, 4H), 3.40 (s, 1H), 3.19 (t, J=5.0 Hz, 4H).

Step 5: (Preparation of vinyl boronate intermediate).

To a solution of the acetylene (0.51 g, 2.29 mmol) in THF (6 mL) was added catechol borane (0.6 mL, 5.7 mmol), and the mixture was heated under reflux overnight. The cooled reaction mixture was concentrated under reduced pressure, and used without further purification in step 6.

Step 6: (Preparation of 2-{2-[(E)-2-(2,6-difluoro-4-morpholin-4-ylphenyl)vinyl]pyridin-4-yl}-1,5,6,7-tetrahydro-4H-pyrrolo[3,2-c]pyridin-4-one trifluoroacetate).

This compound was prepared in 4% yield by the cross coupling of the vinylboronate from step 5 above and 2-(2-Chloropyridin-4-yl)-1,5,6,7-tetrahydro-4H-pyrrolo[3,2-c]pyridin-4-one according to the general procedure described for Example 551: mp >300° C.; ¹H NMR (300 MHz, CD₃OD) δ 8.36 (d, J=6.4 Hz, 1H), 8.25 (d, J=1.6 Hz, 1H), 7.83 (d, J=16.7 Hz, 1H), 7.80-7.79 (m, 1H), 7.46 (s, 1H), 7.30 (d, J=16.6 Hz, 1H), 6.68 (d, J=13.5 Hz, 2H), 3.82-3.81 (m, 4H), 3.61 (t, J=6.9 Hz, 2H), 3.34-3.29 (m, 4H), 3.02 (t, J=6.9 Hz, 2H); ESI-MS m/z 437 [M+H]⁺.

### EXAMPLE 740

This example illustrates the preparation of 2-{2-[(E)-2-(3-fluoro-2-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 3-fluoro-2-(morpholin-4-yl)benzaldehyde).

This compound was prepared in 75% yield by a procedure similar to the one described in step 1 of the synthesis of Example 707 using morpholine and 2,3-difluorobenzaldehyde: ¹H NMR (300 MHz, DMSO-d₆) δ 10.43 (s, 1H), 7.58-7.51 (m, 2H), 7.34-7.31 (m, 1H), 3.73 (t, J=4.5 Hz, 4H), 3.17-3.13 (m, 4H).

Step 2: (Preparation of vinyl boronate intermediate).

The above intermediate was prepared in 37% yield by a procedure similar to the one described in step 2 of the synthesis of Example 707 using the 3-fluoro-2-(morpholin-4-yl)benzaldehyde obtained in step 1.

Step 3: (Preparation of 2-{2-[(E)-2-(3-fluoro-2-morpholin-4-ylphenyl)vinyl]pyridin-4-yl}-1,5,6,7-tetrahydro-4H-pyrrolo[3,2-c]pyridin-4-one trifluoroacetate).

This compound was prepared in 7% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 224-227° C.; ¹H NMR (300 MHz, DMSO-d₆) δ 12.38 (s, 1H), 8.62 (d, J=6.1 Hz, 1H), 8.23 (s, 1H), 8.18 (d, J=16.5 Hz, 1H), 7.81 (s, 1H), 7.58-7.55 (m, 1H), 7.45 (s, 1H), 7.30-7.25 (m, 4H), 3.78 (t, J=4.2 Hz, 4H), 3.46-3.42 (m, 2H), 3.11-2.99 (m, 4H), 2.92 J=6.8 Hz, 2H); ESI-MS m/z 419 [M+H]⁺.

### EXAMPLE 741

This example illustrates the preparation of 2-(2-{(E)-2-[4-(2-morpholin-4-ylethyl)phenyl]vinyl}pyridin-4-yl)-1,5,6,7-tetrahydro-4H-pyrrolo[3,2-c]pyridin-4-one trifluoroacetate.

Step 1: (Preparation of 4-(4-bromophenylacetyl)morpholine).

The above intermediate was prepared following a procedure similar the one described in step 1 of the compound synthesis described in Example 720 using 4-bromophenylacetic acid and morpholine. The crude product was used without purification in the next step; ¹H NMR (300 MHz, DMSO-d₆) δ 7.48 (d, J=8.4 Hz, 2H), 7.17 (d, J=8.4 Hz, 2H), 3.70 (s, 2H), 3.57-3.38 (m, 8H).

Step 2: (Preparation of 4-[2(4-bromophenyl)ethyl]morpholine).

The above compound was prepared following a procedure similar the one described in step 2 of the synthesis of Example 734 using the intermediate obtained in step 1. The reaction crude product was used without purification in the next step; ¹H NMR (300 MHz, CDCl₃) δ 7.39 (d, J=8.3 Hz, 2H), 7.07 (d, J=8.3 Hz, 2H), 3.73 (t, J=4.6 Hz, 4H), 7.97-6.98 (m, 2H), 2.63-2.42 (m, 6H).

Step 3: (Preparation of 4-[2-(4-morpholino)ethyl]benzaldehyde).

The above intermediate was prepared following a procedure similar to the one used in step 2 of the compound synthesis of Example 733. The crude product was used without purification in the next step.

Step 4: (Preparation of vinyl boronate intermediate).

The above intermediate was prepared in 55% yield by a procedure similar to the one described in step 2 of the synthesis of Example 707 using the 4-[2-(4-morpholino)ethyl]benzaldehyde obtained in step 3.

Step 5: (Preparation of 2-(2-{(E)-2-[4-(2-morpholin-4-ylethyl)phenyl]vinyl}pyridin-4-yl)-1,5,6,7-tetrahydro-4H-pyrrolo[3,2-c]pyridin-4-one trifluoroacetate).

This compound was prepared in 18% yield by the cross coupling of the vinylboronate from step 4 above and 2-(2-Chloropyridin-4-yl)-1,5,6,7-tetrahydro-4H-pyrrolo[3,2-c]pyridin-4-one according to the general procedure described for Example 551: mp 123-128° C.; ¹H NMR (300 MHz, CD₃OD) δ 8.45 (d, J=6.5 Hz, 1H), 8.35 (d, J=1.6 Hz, 1H), 7.97-7.81 (m, 2H), 7.72 (d, J=8.1 Hz, 2H), 7.51 (s, 1H), 7.43 (d, J=8.1 Hz, 2H), 7.29 (d, J=16.4 Hz, 1H), 4.20-4.05 (m, 2H), 3.92-3.78 (m, 2H), 3.61 (t, J=6.9 Hz, 4H), 3.52-3.40 (m, 3H), 3.21-3.10 (m, 3H), 3.03 (t, J=6.9 Hz, 2H); ESI-MS m/z 429 [M+H]⁺.

### EXAMPLE 742

This example illustrates the preparation of 2-(2-{(E)-2-[2-morpholin-4-yl-4-(morpholin-4-ylmethyl)phenyl]vinyl}pyridin-4-yl)-1,5,6,7-tetrahydro-4H-pyrrolo[3,2-c]pyridin-4-one trifluoroacetate. Step 1: (Preparation of 2-[2-(morpholin-4-yl)-4-(morpholin-4-ylcarbonyl)phenyl]1,3-dioxolane).

The above intermediate was prepared following a procedure similar to the one described in step 1 of the compound synthesis of Example 732 starting from the product obtained in step 1 of the compound synthesis of Example 726 and ethylene glycol. The crude product was carried out to the next step without purification: ¹H NMR (300 MHz, CDCl₃) δ 10.30 (s, 1H), 7.84 (d, J=7.8 Hz, 1H), 7.15 (d, J=1.2 Hz, 1H), 7.10 (dd, J=7.8, 0.7 Hz, 1H), 5.30 (s, 1H), 3.09 (t, J=4.5 Hz, 4H), 3.83-3.30 (m, 10H), 3.15-3.05 (m, 4H).

Step 2: (Preparation of 2-[2-(morpholin-4-yl)-4-(morpholin-4-ylmethyl)phenyl]1,3-dioxolane).

The above compound was prepared following a procedure similar the one described in step 2 of the synthesis of Example 734 using the intermediate obtained in step 1. The reaction crude product was used without purification in the next step.

Step 3: (Preparation of 2-(morpholin-4-yl)-4-(morpholin-4-ylmethyl)benzaldehyde).

To a solution of the intermediate (˜11 mmol) obtained in step 2 in THF (16 mL) was added a concentrated solution of HCl (4 mL). The mixture was stirred at room temperature for 30 min and was basified with a 2 N NaOH solution till pH˜9. The solution thus obtained was extracted with CH₂Cl₂ (3×60 mL). The combined organic phase was dried (Na₂SO₄) and concentrated. Purification by flash chromatography (eluent 96:4 CH₂Cl₂/MeOH) gave the above aldehyde (2.28 g, 66% over 3 steps): ¹H NMR (300 MHz, CDCl₃) δ 10.28 (s, 1H), 7.76 (d, J=7.8 Hz, 1H), 7.18-7.05 (m, 2H), 3.90 (t, J=4.5 Hz, 4H), 3.72 (t, J=4.6 Hz, 4H), 3.52 (s, 2H), 3.15-3.03 (m, 4H), 2.45 (t, J=4.5 Hz, 4H).

Step 4: (Preparation of vinyl boronate intermediate).

The above intermediate was prepared in 40% yield by a procedure similar to the one described in step 2 of the synthesis of Example 707 using the 2-(morpholin-4-yl)-4-(morpholin-4-ylmethyl)benzaldehyde obtained in step 3.

Step 5: (Preparation of 2-(2-{(E)-2-[2-morpholin-4-yl-4-(morpholin-4-ylmethyl) phenyl]vinyl}pyridin-4-yl)-1,5,6,7-tetrahydro-4H-pyrrolo[3,2-c]pyridin-4-one trifluoroacetate).

This compound was prepared in 23% yield by the cross coupling of the vinylboronate from step 4 above and 2-(2-Chloropyridin-4-yl)-1,5,6,7-tetrahydro-4H-pyrrolo[3,2-c]pyridin-4-one according to the general procedure described for Example 551: mp 145-150° C.; ¹H NMR (300 MHz, CD₃OD) δ 8.48 (d, J=6.5 Hz, 1H), 8.29 (d, J=1.5 Hz, 1H), 8.11 (d, J=16.5,1H), 7.87 (dd, J=6.5,1.8 Hz, 1H), 7.82 (d, J=7.9 Hz, 1H), 7.52 (s, 1H), 7.40-7.27 (m, 3H), 4.39 (s, 2H), 4.20-3.75 (m, 10H), 3.68-3.58 (m, 3H), 3.10-2.99 (m, 7H); ESI-MS m/z 500 [M+H]⁺.

### EXAMPLE 743

This example illustrates the preparation of 2-{2-[(E)-2-(3-fluoro-4-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 3-flouro-4-morpholin-4-yl-benzaldehyde).

3-Fluoro-4-morpholin-4-yl-benzaldehyde was prepared in 89% yield by a procedure similar to the one described in step 1 of the compound synthesis of Example 707 using morpholine and 3,4-difluorobenzaldehyde. ¹H NMR (300 MHz, CDCl₃) δ 9.24 (d, J=2.1 Hz, 1H), 7.62-7.50 (m, 2H), 6.99 (t, J=8.3 Hz, 1H), 3.88 (t, J=4.8 Hz, 4H), 3.26 (t, J=4.8 Hz, 4H).

Step 2: (Preparation of vinyl boronate intermediate).

The above intermediate was prepared in 38% yield by a procedure similar to the one described in step 2 of the compound synthesis of Example 707 using the 3-flouro-4-morpholin-4-yl-benzaldehyde obtained in step 3.

Step 3: (Preparation of 2-{2-[(E)-2-(3-fluoro-4-morpholin-4-ylphenyl)vinyl]pyridin-4-yl}-1,5,6,7-tetrahydro-4H-pyrrolo[3,2-c]pyridin-4-one trifluoroacetate).

This compound was prepared in 78% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 220-224° C.; ¹H NMR (300 MHz, DMSO-d₆) δ 12.34 (s, 1H), 8.57 (d, J=6.2 Hz, 1H), 8.25 (s, 1H), 7.83 (d, J=16.4 Hz, 1H), 7.78 (d, J=5.7 Hz, 1H), 7.52-7.34 (m, 3H), 7.24 (s, 1H), 7.19-7.09 (m, 2H), 3.76 (t, J=4.8 Hz, 4H), 3.46-3.43 (m, 2H), 3.05-3.15 (m, 4H), 2.92 (t, J=6.8 Hz, 2H); ESI-MS m/z 419 [M+H]⁺.

### EXAMPLE 744

This example illustrates the preparation of 2-{2-[(E)-2-(3,5-difluoro-4-morpholin-4-ylphenyl)vinyl]pyridin-4-yl}-1,5,6,7-tetrahydro-4H-pyrrolo[3,2-c]pyridin-4-one trifluoroacetate.

Step 1: (Preparation of 3,5-diflouro-4-morpholin4-yl-benzaldehyde).

3,5-Diflouro-4-morpholin4-yl-benzaldehyde was prepared in 66% yield by a procedure similar to the one described in step 1 of the synthesis of Example 707 using morpholine and 3,4,5-trifluorobenzaldehyde. ¹H NMR (300 MHz, CDCl₃) δ 9.79 (t, J=1.7 Hz, 1H), 7.45-7.20 (m, 2H), 3.82 (t, J=4.7 Hz, 4H), 3.40-3.30 (m, 4H).

Step 2: (Preparation of vinyl boronate intermediate).

The above intermediate was prepared in 51% yield by a procedure similar to the one described in step 2 of the synthesis of Example 707 using the 3,5-diflouro-4-morpholin-4-yl-benzaldehyde obtained in step 1.

Step 3: (Preparation of 2-{2-[(E)-2-(3,5-difluoro-4-morpholin-4-ylphenyl)vinyl]pyridin-4-yl}-1,5,6,7-tetrahydro-4H-pyrrolo[3,2-c]pyridin-4-one trifluoroacetate).

This compound was prepared in 5% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: mp 238-242° C.; ¹H NMR (300 MHz, CD₃OD) δ 8.45 (d, J=6.5 Hz, 1H), 8.27 (d, J=1.7 Hz, 1H), 7.82 (dd, J=4.6, 1.9 Hz, 1H), 7.72 (d, J=16.3 Hz, 1H), 7.49 (s, 1H), 7.35-7.25 (m, 2H), 7.17 (d, J=16.3 Hz, 1H), 3.80 (t, J=4.8 Hz, 4H), 3.62 (t, J=7.0 Hz, 2H), 3.30-3.20 (m, 4H), 3.03 (t, J=7.0 Hz, 2H); ESI-MS m/z 437 [M+H]⁺.

### EXAMPLE 745

This example illustrates the preparation of 2-{2-[(E)-2-(2-aminophenyl)vinyl]pyridin-4-yl}-1,5,6,7-tetrahydro-4H-pyrrolo[3,2-c]pyridin-4-one trifluoroacetate.

Step 1: (Preparation of 2-(1,4-dioxa-8-azaspiro[4.5]dec-8-yl)benzaldehyde).

The above compound was prepared in 96% yield by a procedure similar to the one described in step 1 of the synthesis of Example 683 using 2-fluorobenzaldehye and 1,4-dioxa-8-azaspiro[4.5]-decane: ¹H NMR (300 MHz, CDCl₃) δ 10.33 (s, 1H), 7.80 (dd, J=7.5, 1.5 Hz, 1H), 7.52-7.48 (m, 1H), 7.14-7.08 (m, 2H), 4.00 (s, 4H), 3.19 (t, J=5.5 Hz, 4H), 1.91 (t, J=5.5 Hz, 4H).

Step 2: (Preparation of vinyl boronate intermediate).

The above intermediate was prepared in 64% yield by a procedure similar to the one described in step 2 of the synthesis of Example 707 using the 2-(1,4-dioxa-8-azaspiro[4.5]dec-8-yl)benzaldehyde obtained in step 1.

Step 3: (Preparation of 2-{2-[(E)-2-(2-(1,4-dioxa-8-azaspiro[4.5]dec-8-yl)phenyl)vinyl]pyridin-4-yl}-1,5,6,7-tetrahydro-4H-pyrrolo[3,2-c]pyridin-4-one trifluoroacetate).

This compound was prepared in 17% yield by the cross coupling of the vinylboronate from step 2 above and 2-(2-Chloropyridin-4-yl)-1,5,6,7-tetrahydro-4H-pyrrolo[3,2-c]pyridin-4-one according to the general procedure described for Example 551: ¹H NMR (300 MHz, DMSO-d₆) δ 12.10-11.80 (brs, 1H), 8.50 (d, J=5.2 Hz, 1H), 7.98 (d, J=16.2 Hz, 1H), 7.76 (br s, 1H), 7.67 (d, J=6.7 Hz, 1H), 7.46 (dd, J=5.2, 1.4 Hz, 1H), 7.20-7.06 (m, 5H), 7.02 (s, 1H), 3.92 (s, 4H), 3.42-3.39 (m, 2H), 3.00-2.90 (m, 4H), 2.86 (t, J=6.8 Hz, 2H), 1.84-1.82 (m, 4H).

Step 4: (Preparation of 2-{2-[(E)-2-(2-aminophenyl)vinyl]pyridin-4-yl}-1,5,6,7-tetrahydro-4H-pyrrolo[3,2-c]pyridin-4-one trifluoroacetate).

A solution of the ketal (0.17 g, 0.37 mmol) from step 3 above in 3:3:2 THF/MeOH/concd HCl (8 mL) was heated under reflux for 5 h. The cooled reaction mixture was basified with 2 N NaOH (pH 9-10), and the product was extracted into CH₂Cl₂ (3×50 mL). The organic extract was concentrated under reduced pressure to a volume of 10 mL, and then stirred with TFA (1.5 mL) for 1 h. The solution was concentrated under reduced pressure. Purification of the crude product by preparative HPLC gave Example 745 (a salt containing 1.75 equivalent of TFA, 47 mg, 23%) as a yellow solid: mp 180-183° C.; ¹H NMR (300 MHz, DMSO-d₆) δ 12.47 (br s, 1H), 8.60 (d, J=6.5 Hz, 1H), 8.45 (br s, 1H), 8.08 (d, J=16.1 Hz, 1H), 7.85 (d, J=6.1 Hz, 1H), 7.60 (s, 1H), 7.45 (d, J=6.9 Hz, 1H), 7.31 (s, 1H), 7.30-7.15 (m, 1H), 7.03 (d, J=16.1 Hz, 1H), 6.75 (d, J=8.2 Hz, 1H), 6.70-6.60 (m, 1H), 3.46-3.43 (m, 2H), 2.94 (t, J=6.8 Hz, 2H); ESI-MS m/z 331 [M+H]⁺.

### EXAMPLE 746

This example illustrates the preparation of 2-(2-{(E)-2-[2-(4-oxopiperidin-1-yl)phenyl]vinyl}pyridin-4-yl)-1,5,6,7-tetrahydro-4H-pyrrolo[3,2-c]pyridin-4-one trifluoroacetate.

A solution of the ketal intermediate (85 mg, 0.19 mmol) from step 3 in the synthesis of Example 745 in 80% aqueous AcOH (5 mL) was heated at 65° C. overnight. The cooled reaction mixture was concentrated under reduced pressure. Purification of the crude product by preparative HPLC gave an orange solid (62% yield): mp 182-185° C; ¹H NMR (300 MHz, DMSO-d₆) δ 12.42 (br s, 1H), 8.62 (d, J=6.3 Hz, 1H), 8.39 (br s, 1H), 8.17 (d, J=16.4 Hz, 1H), 7.87 (d, J=6.3 Hz, 1H), 7.69 (d, J=7.5 Hz, 1H), 7.57 (br s, 1H), 7.43-7.20 (m, 5H), 3.46-3.43 (m, 2H), 3.28 (t, J=5.8 Hz, 4H), 2.93 (t, J=6.7 Hz, 2H), 2.60 (t, J=5.6 Hz, 4H); ESI-MS m/z 413 [M+H]⁺.

### EXAMPLE 747

This example illustrates the preparation of 2-{2-[(E)-2-(2-piperazin-1-ylphenyl)vinyl]pyridin-4-yl}-1,5,6,7-tetrahydro-4H-pyrrolo[3,2-c]pyridin-4-one trifluoroacetate.

Step 1: (Preparation of 2-piperazin-1-yl-benzaldehyde).

2-Piperazin-1-yl-benzaldehyde was prepared in 76% yield by a procedure similar to the one described in step 1 in the synthesis of Example 707 using 2-fluorobenzaldehyde and piperazine.

Step 2: (Preparation of 1,1-dimethylethyl 4-(2-formylphenyl)piperazine-1-carboxylate ).

To a solution of di-tert-butyl-dicarbonate (4.22 g, 19.4 mmol) in CH₂Cl₂ (35 mL), was added 2-piperazin-1-yl-benzaldehyde (2.45 g, 12.9 mmol) obtained in step 1. The mixture was stirred overnight at room temperature. The reaction mixture was diluted with water (30 mL) and extracted with CH₂Cl₂ (4×40 mL). The organic phase was separated and washed with brine, dried (Na₂SO₄) and concentrated under reduced pressure. Purification by flash column chromatography (eluent 93:7 hexanes/EtOAc to 4:5 hexanes/EtOAc) gave the above compound as a yellow oil (2.67 g, 71%): ¹H NMR (300 MHz, CDCl₃) δ 10.35 (s, 1H), 7.83 (dd, J=7.7,1.7 Hz, 1H), 7.57-7.51 (m, 1H), 7.27-7.09 (m, 2H), 3.63 (t, J=5.0 Hz, 4H), 3.04 (t, J=5.0 Hz, 4H), 1.49 (s, 9H)

Step 3: (Preparation of vinyl boronate intermediate).

The above intermediate was prepared in 22% yield by a procedure similar to the one described in step 2 of the synthesis of Example 707 using the intermediate obtained in step 2 above.

Step 4: (Preparation of 2-{2-[(E)-2-(2-piperazin-1-ylphenyl)vinyl]pyridin-4-yl}-1,5,6,7-tetrahydro-4H-pyrrolo[3,2-c]pyridin-4-one trifluoroacetate).

A mixture of 2-(2-Chloropyridin-4-yl)-1,5,6,7-tetrahydro-4H-pyrrolo[3,2-c]pyridin-4-one (351 mg, 1.42 mmol), the vinylboronate obtained in step 3 (470 mg, 1.13 mmol), an aqueous solution of 2 M Cs₂CO₃ (2.9 mL, 5.8 mmol) and DMF (6 mL) was degassed (3×, vacuum/argon). To this mixture was added Pd(PPh₃)₄ (65 mg, 0.057 mmol). The resulting mixture was degassed (3×, vacuum/argon) and then heated overnight at 80° C. The cooled reaction mixture was filtered, and the filter cake was washed with CH₂Cl₂ (30 mL). The filtrate was treated with water (10 mL) and extracted with CH₂Cl₂ (3×20 mL). The combined organic phase was dried (Na₂SO₄) and concentrated under reduced pressure. The residue was then taken up in a solution of TFA (5 mL) in CH₂Cl₂ (10 mL) and stirred overnight at room temperature. The reaction mixture was concentrated under reduced pressure and purified by preparative HPLC to give a yellow solid: yield 7%, mp 178-183° C.; ¹H NMR (300 MHz, DMSO-d₆) δ 12.34 (s, 1H), 8.87-8.80 (m, 2H), 8.61 (d, J=5.9 Hz, 1H), 8.24 (s, 1H), 8.03 (d, J=16.4 Hz, 1H), 7.76, (s, 1H), 7.70 (d, J=7.6 Hz, 1H), 7.45-7.40 (m, 2H), 7.29-7.19 (m, 4H), 3.46-3.42 (m, 2H), 3.36-3.31 (m, 4H), 3.15-3.10 (m, 4H), 2.91 (t, J=6.7 Hz, 2H); ESI-MS m/z 400 [M+H]⁺.

### EXAMPLE 748

This example illustrates the preparation of 2-(2-{(E)-2-[3-(morpholin-4-ylmethyl)phenyl]vinyl}pyridin-4-yl)-1,5,6,7-tetrahydro-4H-pyrrolo[3,2-c]pyridin-4-one trifluoroacetate.

Step 1: (Preparation of 4-(3-bromobenzyl)morpholine).

To an ice-cold solution of morpholine (1.92 g, 22 mmol) 3-bromobenzyl bromide (5.0 g, 20 mmol) in DMF (20 mL) was added NaH (1.6 g, 40 mmol, 60% suspended in mineral oil) and the suspension was stirred for 30 min. To this mixture was added a solution of 3-bromobenzyl bromide (5.0 g, 20 mmol) in DMF (4 mL). The resulting mixture was stirred for 2 h at 0° C.. The reaction mixture was diluted with EtOAc (200 mL), washed with saturated aqueous NH₄OH, dried (Na₂SO₄) and concentrated under reduced pressure. Purification by flash chromatography (eluent 3:1 hexanes/EtOAc) gave 4-(3-bromobenzyl)morpholine (4.3 g, 84%) as clear oil. ¹H NMR (300 MHz, CDCl₃) δ 7.50 (s, 1H), 7.37 (d, J=6.3 Hz, 1H), 7.30-7.15 (m, 2H), 3.71 (t, J=4.8 Hz, 4H), 3.46 (s, 2H), 2.44 (t, J=4.8 Hz, 4H).

Step 2: (Preparation of 3-(morpholin-4-ylmethyl)benzaldehyde).

To a solution of 4-(3-bromobenzyl)morpholine obtained in step 1 (2.55 g, 10 mmol) in THF (20 mL) at −78° C. was added t-BuLi (13 mL, 22 mmol, 1.7 M solution in pentanes). After 2 h, DMF (1.7 mL, 22 mmol) was added to the reaction mixture, which was then warmed up to room temperature and stirred for 1 h. The reaction mixture was diluted with EtOAc (150 mL) and washed with saturated NH₄Cl, brine, dried (Na₂SO₄) and concentrated under reduced pressure. Purification by flash chromatography (eluent 3:1 hexanes/EtOAc) gave 3-(morpholin-4-ylmethyl)benzaldehyde (1.19 g, 58%) as yellow oil. ¹H NMR (300 MHz, CDCl₃) δ 10.0 (s, 1H), 7.86 (s, 1H), 7.78 (d, J=7.5 Hz, 1H), 7.62 (d, J=7.5 Hz, 1H), 7.49 (t, J=7.5 Hz, 1H), 3.72 (t, J=4.8 Hz, 4H), 3.57 (s, 2H), 2.46 (t, J=4.4 Hz, 4H).

Step 3: (Preparation of vinyl boronate intermediate).

The above compound was prepared in 9% yield from 3-(morpholin-4-ylmethyl)benzaldehyde obtained in step 2 above using a procedure similar to the one described in step 2 of the compound synthesis of Example 707.

Step 4: (Preparation of 2-(2-{(E)-2-[3-(morpholin-4-ylmethyl)phenyl]vinyl}pyridin-4-yl)-1,5,6,7-tetrahydro-4H-pyrrolo[3,2-c]pyridin-4-one trifluoroacetate).

This compound was prepared in 9% yield by the cross coupling of the vinylboronate from step 3 above and 2-(2-Chloropyridin-4-yl)-1,5,6,7-tetrahydro-4H-pyrrolo[3,2-c]pyridin-4-one according to the general procedure described for Example 551: ¹H NMR (300 MHz, CD₃OD) δ 8.49 (d, J=6.4 Hz, 1H), 8.32 (s, 1H), 7.95-7.78 (m, 4H), 7.67-7.54 (m, 2H), 7.50 (s, 1H), 7.37 (d, J=16.5 Hz, 1H), 4.45 (s, 2H), 4.06-3.90 (m, 4H), 3.62 (t, J=6.8 Hz, 2H), 3.33 (t, J=6.8 Hz, 2H); m/z 415 [M+H]⁺.

### EXAMPLE 749

This example illustrates the preparation of 2-(2-{(E)-2-[3-(2-morpholin-4-ylethyl) phenyl]vinyl}pyridin-4-yl)-1,5,6,7-tetrahydro-4H-pyrrolo[3,2-c]pyridin-4-one trifluoroacetate.

Step 1: (Preparation of 4-(3-bromophenylacetyl)morpholine).

To a solution of 3-bromobenzoacetic acid (5.00 g, 23.3 mmol) in CH₂Cl₂ (40 mL) was added, sequentially, EDCI (8.93 g, 46.6 mmol), HOBT (4.72 g, 35.0 mmol), morpholine (2.53 g, 29.1 mmol), and diisopropylethylamine (3.76 g, 29.1 mmol). The reaction mixture was stirred for 2 hours at room temperature, after which the mixture was diluted with water (40 mL) and extracted with CH₂Cl₂ (3×25 mL). The combined organic phase was dried (Na₂SO₄) and concentrated under reduced pressure. Purification by flash column chromatography (eluent 95:5 CH₂Cl₂/MeOH) gave the above compound as an oil (4.50 g, 68%).

Step 2: (Preparation of 4-[2-(3-bromophenyl)ethy]morpholine).

To a solution of the material obtained in step 1 (4.50 g, 15.80 mmol) in THF (47 mL) was added 1.0 M BH₃ (47.7 mL, 47.7 mmol, 1 M in THF). The solution was stirred at reflux for 3 hours, after which the solution was cooled to 0° C. and quenched with MeOH (25 mL). The reaction mixture was then concentrated and suspended in 1:1 MeOH/2 N HCl (aq) and stirred at refulx for 3 hours. The reaction mixture was then basified with 2 N NaOH (aq, 70 mL) and extracted with CH₂Cl₂ (3×50 mL). The combined organic phase was dried (Na₂SO₄) and concentrated under reduced pressure. Purification by flash column chromatography (eluent 90:4.5:0.5 CH₂Cl₂/MeOH/NH₄OH) gave the above compound as an oil (2.99 g, 70%): ¹H NMR (300 MHz, CDCl₃) δ 7.36-7.32 (m, 2H), 7.19-7.12 (m, 2H), 3.74 (t, J=4.6 Hz, 4H), 2.80-2.75 (m, 2H), 2.60-2.55 (m, 2H), 2.51 (t, J=4.6 Hz, 4H).

Step 3: (Preparation of 3-[2-(morpholin-4-yl)ethyl]benzaldehyde).

To a solution of the material obtained in step 2 (2.99 g, 11.1 mmol) in THF (51 mL) at −78° C. was added tert-butyl lithium (13.1 mL, 22.2 mmol, 1.7 M in THF). Upon stirring for 1 hour, the solution was brought to room temperature, after which DMF (1.62 g, 22.2 mmol) was added and stirred overnight. The reaction mixture was then diluted with water (75 mL) and extracted with CH₂Cl₂ (4×50 mL). The combined organic phase was dried (Na₂SO₄) and concentrated under reduced pressure. Purification by flash column chromatography (eluent EtOAc) gave the above compound as an oil (1.57 g, 65%): ¹H NMR (300 MHz, CDCl₃) δ 10.01 (s, 1H), 7.74-7.71 (m, 1H), 7.49-7.46 (m, 1H), 7.29-7.20 (m, 2H), 3.75 (t, J=4.6 Hz, 4H), 2.89 (t, J=7.9 Hz, 2H), 2.63 (t, J=7.9 Hz, 2H), 2.53 (t, J=4.6 Hz, 4H).

Step 4: (Preparation of vinyl boronate intermediate).

The above compound was prepared in 64% yield from 3-[2-(morpholin-4-yl)ethyl]benzaldehyde obtained in step 3 above using a procedure similar to the one described in step 2 of the synthesis of Example 707.

Step 5: (Preparation of 2-(2-{(E)-2-[3-(2-morpholin-4-ylethyl)phenyl]vinyl}pyridin-4-yl)-1,5,6,7-tetrahydro-4H-pyrrolo[3,2-c]pyridin-4-one trifluoroacetate).

This compound was prepared in 15% yield by the cross coupling of the vinylboronate from step 4 above and 2-(2-Chloropyridin-4-yl)-1,5,6,7-tetrahydro-4H-pyrrolo[3,2-c]pyridin-4-one according to the general procedure described in Example 551: mp 152-155° C.; ¹H NMR (300 MHz, CD₃OD) δ 8.47 (d, J=6.5 Hz, 1H), 8.31 (s, 1H), 7.90-7.81 (m, 2H), 7.67-7.65 (m, 2H), 7.51-7.39 (m, 3H), 7.33 (d, J=16.4 Hz, 1H), 4.07-3.84 (m, 4H), 3.62 (t, J=7.0 Hz, 2H), 3.47 (t, J=8.6 Hz, 2H), 3.31 (m, 4H), 3.16 (t, J=8.6 Hz, 2H), 3.03 (t, J=7.0 Hz, 2H); ESI-MS m/z 429 [M+H]⁺.

### EXAMPLE 750

A mixture of a BOC or trityl protected amine (100-150 mg) in 1:5 TFA/CH₂Cl₂ (3.00 mL) was stirred at ambient temperature. BOC deprotection was monitored to completion by reverse phase HPLC over several hours, then the mixture was concentrated and purified by reverse-phase C18 chromatography with a water/acetonitrile gradient. Purified compounds were assayed by analytical reverse phase HPLC, NMR, and MS. The following compounds were prepared with this general method.

| | | Calculated | Found | Found |
|---|---|---|---|---|
| Example | | Exact Mass | Exact Mass | Electrospray |
| No. | Compound | m + H | m + H | m + H |
| EXAMPLE 751 | 2-{2-[(E)-2-(1H-indol-5- | 355.1553 | 355.1537 | 355 |
| | yl)ethenyl]pyridin-4-yl}- | | | |
| | 1,5,6,7-tetrahydro-4H- | | | |
| | pyrrolo[3,2-c]pyridin-4-one | | | |
| | trifluoroacetate | | | |
| EXAMPLE 752 | 2-{2-[(E)-2-(1H-indol-3- | 355.1553 | 355.1549 | 355 |
| | yl)ethenyl]pyridin-4-yl}- | | | |
| | 1,5,6,7-tetrahydro-4H- | | | |
| | pyrrolo[3,2-c]pyridin-4-one | | | |
| | trifluoroacetate | | | |
| EXAMPLE 753 | 2-{2-[(Z)-2-(1H-imidazol- | 306.1349 | 306.1363 | 306 |
| | 4-yl)ethenyl]pyridin-4-yl}- | | | |
| | 1,5,6,7-tetrahydro-4H- | | | |
| | pyrrolo[3,2-c]pyridin-4-one | | | |
| | trifluoroacetate | | | |
| EXAMPLE 754 | 2-{2-[(E)-2-(1H-imidazol-4- | 306.1349 | 306.1367 | 306 |
| | yl)ethenyl]pyridin-4-yl}- | | | |
| | 1,5,6,7-tetrahydro-4H- | | | |
| | pyrrolo[3,2-c]pyridin-4-one | | | |
| | trifluoroacetate | | | |

### EXAMPLE 755

This example illustrates the preparation of 2-{2-[(E)-2-(4-hydroxyphenyl)ethenyl]pyridin-4-yl}-1,5,6,7-tetrahydro-4H-pyrrolo[3,2-c]pyridin-4-one.

A 1 M solution of BBr₃ in CH₂Cl₂ (2.5 mL, 2.5 mmol) was added to a −20° C. cooled mixture of 2-{2-[(E)-2-(4-methoxyphenyl)ethenyl]pyridin-4-yl}-1,5,6,7-tetrahydro-4H-pyrrolo[3,2-c]pyridin-4-one (86 mg, 0.25 mmol) in CH₂Cl₂ (1.00 mL). The resulting mixture was warmed to ambient temperature and monitored to completion by HPLC and LCMS, then was cooled and quenched by careful addition of excess methanol followed with excess 30% ammonium hydroxide (1.00 mL). After concentration to a small volume, the solid was collected by filtration, washed with water, and vacuum dried to afford 73 mg of Example 755, which was characterized by analytical reverse phase HPLC, NMR, and MS. Calculated Exact Mass 332.1399 (M+H⁺); Found Positive Electrospray LC-MS, m/e 332 (M+H⁺).

### EXAMPLE 756

Step 1: Preparation of 1-fluoro-2-arylvinylbromides (E/Z mixture).

1-Fluoro-2-arylvinylbromides were prepared by the literature procedure (J. Xu and D. J. Burton, Tetrahedron Lett., 43, 2877 (2002)) by the condensation of aryl aldehydes with fluorotribromomethane in the presence of triphenylphosphine as a mixture of E/Z isomers. The products (E/Z mixture) were purified by flash chromatography on silica gel and characterized by GC-MS, ¹H NMR, ¹³C NMR, ¹⁹F NMR and HR-MS.

Step 2: Preparation of the 1-fluorovinyldioxoborolanes.

The 1-fluorovinyldioxoborolanes were prepared by the literature procedure (T. Ishiyama, M. Murata, and N. Miyaura, J. Org. Chem., 60, 7508 (1995)) by the palladium-catalyzed cross-coupling of the bis-(pinacolato)diboron with 1-fluoro-2-arylvinylbromides. The products were characterized by GC-MS, ¹H NMR, and ¹⁹F NMR and used as such.

Step 3: Preparation of the 1-fluorostyrenyl compounds.

The following 1-fluorostyrenyl compounds were prepared by the general procedure of the cross-coupling of the 1-fluorovinyldioxoborolanes with the 2-(2-chloropyridin-4-yl)-1,5,6,7-tetrahydro-4H-pyrrolo[3,2-c]pyridin-4-one in DMSO or DMF. The crude reaction mixture was purified by reverse phase C18 HPLC with a water/acetonitrile gradient containing 0.1% TFA to separate the E- and Z-isomers. The purified products (E and Z isomers) were characterized by analytical reverse phase HPLC, LC-MS, ¹H NMR, ¹³C NMR, ¹⁹F NMR and HR-MS.

| | | Calculated | Found |
|---|---|---|---|
| Example | | Exact Mass | Exact Mass |
| No. | Compound | (m + H) | (m + H) |
| EXAMPLE 757 | 2-{2-[(Z)-1-fluoro-2- | 334.135 | 334.1364 |
| | phenylvinyl]pyridin-4-yl}-1,5,6,7- | | |
| | tetrahydro-4H-pyrrolo[3,2- | | |
| | c]pyridin-4-one trifluoroacetate | | |
| EXAMPLE 758 | 2-{2-[(Z)-1-fluoro-2-(2- | 348.1507 | 348.1532 |
| | methylphenyl)vinyl]pyridin-4-yl}- | | |
| | 1,5,6,7-tetrahydro-4H-pyrrolo[3,2- | | |
| | c]pyridin-4-one trifluoroacetate | | |
| EXAMPLE 759 | 2-{2-[(E)-1-fluoro-2-(2- | 348.1507 | 348.1501 |
| | methylphenyl)vinyl]pyridin-4-yl}- | | |
| | 1,5,6,7-tetrahydro-4H-pyrrolo[3,2- | | |
| | c]pyridin-4-one trifluoroacetate | | |
| EXAMPLE 760 | 2-{2-[(Z)-2-(2-chlorophenyl)-1- | 368.0973 | 368.096 |
| | fluorovinyl]pyridin-4-yl}-1,5,6,7- | | |
| | tetrahydro-4H-pyrrolo[3,2- | | |
| | c]pyridin-4-one trifluoroacetate | | |
| EXAMPLE 761 | 2-{2-[(E)-1-fluoro-2-(2-morpholin- | 419.1878 | 419.1841 |
| | 4-ylphenyl)vinyl]pyridin-4-yl}- | | |
| | 1,5,6,7-tetrahydro-4H-pyrrolo[3,2- | | |
| | c]pyridin-4-one trifluoroacetate | | |
| EXAMPLE 762 | 2-{2-[(2-morpholin-4- | 399.1816 | 399.1801 |
| | ylphenyl)ethynyl]pyridin-4-yl}- | | |
| | 1,5,6,7-tetrahydro-4H-pyrrolo[3,2- | | |
| | c]pyridin-4-one trifluoroacetate | | |
| EXAMPLE 763 | 2-{2-[(E)-1,2-difluoro-2- | 352.1256 | 352.1255 |
| | phenylvinyl]pyridin-4-yl}-1,5,6,7- | | |
| | tetrahydro-4H-pyrrolo[3,2- | | |
| | c]pyridin-4-one trifluoroacetate | | |
| EXAMPLE 764 | methyl 4-{(Z)-2-fluoro-2-[4-(4-oxo- | 392.1405 | 392.1378 |
| | 4,5,6,7-tetrahydro-1H-pyrrolo[3,2- | | |
| | c]pyridin-2-yl)pyridin-2- | | |
| | yl]vinyl}benzoate trifluoroacetate | | |
| EXAMPLE 765 | 4-{(Z)-2-fluoro-2-[4-(4-oxo- | 378.1248 | 378.1245 |
| | 4,5,6,7-tetrahydro-1H-pyrrolo[3,2- | | |
| | c]pyridin-2-yl)pyridin-2- | | |
| | yl]vinyl}benzoic acid | | |
| | trifluoroacetate | | |

### EXAMPLE 766

This example illustrates the preparation of 2-Fluorostyrenyl analogs.

Step 1: Preparation of 2-bromo-1-fluoro-vinyl)-benzene.

To a mixture of 1,3-dibromo-5,5-dimethylhydantoin (4.3 g, 15 mmol) in sulfolane (30 ml), pyridinium HF complex was added. The resulting mixture was cooled on an ice bath, before a solution of phenylacetylene in sulfolane (10 ml) was added over 10 minutes. After the addition, the reaction mixture was stirred for 10 minutes at 0° C., and 20 minutes at room temperature, then poured into ice water and extracted with ether. The organic layer was washed with water, saturated NaHCO₃, brine and dried over Na₂SO₄. Evaporation of ether gave the product as yellow liquid, which was used without further purification.

Step 2: 2-(2-Fluoro-2-phenyl-vinyl)-4,4,5,5-tetramethyl-[1,3,2]dioxaborolane was prepared using the general method described for Example 109 from 2-bromo-1-fluoro-vinyl)-benzene and dipinacolatodiboron.

Step 3: The boronic ester from step 2 above and 2-(2-chloropyridin-4-yl)-1,5,6,7-tetrahydro-4H-pyrrolo[3,2-c]pyridin-4-one were coupled by the general procedure described for Example 551 to produce the following compounds.

| | | Calculated | Found |
|---|---|---|---|
| | | Exact Mass | Exact Mass |
| Example No. | Compound | (m + H) | (m + H) |
| EXAMPLE 767 | 2-{2-[(Z)-2-fluoro-2- | 333.1277 | 334.2 |
| | phenylvinyl]pyridin-4- | | |
| | yl}-1,5,6,7-tetrahydro- | | |
| | 4H-pyrrolo[3,2-c]pyridin- | | |
| | 4-one trifluoroacetate | | |
| EXAMPLE 768 | 2-{2-[(E)-2-fluoro-2- | 333.1277 | 334.1 |
| | phenylvinyl]pyridin-4- | | |
| | yl}-1,5,6,7-tetrahydro- | | |
| | 4H-pyrrolo[3,2-c]pyridin- | | |
| | 4-one | | |

### EXAMPLE 769

This example illustrates the preparation of 2-{2-[(E)-1,2-difluoro-2-phenylvinyl]pyridin-4-yl}-1,5,6,7-tetrahydro-4H-pyrrolo[3,2-c]pyridin-4-one trifluoroacetate.

Step 1: 1,2-difluoro-2-phenylethene (E/Z mixture) was prepared by the literature method (Keith D. Bames and Y. Hu, U.S. Pat. No. 6,207,846).

Step 2: Preparation of tributyl[(Z)]-1,2-difluoro-2-phenylvinyl]stannane

The compound was prepared by a literature procedure (L. Xue, L. Lu; S. D. Pedersen,. Q. Liu; R. M. Narske; D. J. Burton, J. Org. Chem. 62,1064 (1997).

Step 3: Preparation of 2-{2-[(E)-1,2-difluoro-2-phenylvinyl]pyridin-4-yl}-1,5,6,7-tetrahydro-4H-pyrrolo[3,2-c]pyridin-4-one.

A mixture of tributyl[(Z)]-1,2-difluoro-2-phenylvinyl]stannane, 2-(2-chloropyridin-4-yl)-1,5,6,7-tetrahydro-4H-pyrrolo[3,2-c]pyridin-4-one, Pd(Ph₃)₄ and Cu(I)iodide in DMF was heated at 80° C. under N₂ atm overnight. The crude reaction mixture was purified by reverse phase C18 HPLC with a water/acetonitrile gradient containing 0.1% TFA and lyophilized to afford the desired product as a yellow solid. The purified product was characterized by analytical reverse phase HPLC, LC-MS, ¹H NMR, ¹⁹F NMR and HR-MS. Calculated exact mass 352.1256 (m+H); Found 352.1255 (m+H).

### EXAMPLE 770

This example illustrates the preparation of methyl 4-(4-oxo-4,5,6,7-tetrahydro-1H-pyrrolo[3,2-c]pyridin-2-yl)pyridine-2-carboxylate trifluoroacetate.

A suspension of 2-(2-chloropyridin-4-yl)-1,5,6,7-tetrahydro-4H-pyrrolo[3,2-c]pyridin-4-one (200 mg, 0.81 mmol) in 3.0 mL of methanol, 3.0 mL of dimethylformamide and 0.3 mL of triethylamine was treated with palladium acetate (20 mg, 0.09 mmol) and diphenylphosphinopferrocene (60 mg, 0.11 mmol). Carbon monoxide gas was then bubbled into the suspension for 5-10 minutes and then heated to 65° C. for 2 hours. The reaction contents were filtered through a syringe filter (0.45□m), purified by rpHPLC and lyophilized to give the title compound as a yellow solid (39 mg, 0.10 mmol, 12%). ¹H NMR (300 MHz, DMSO-d₆) δ 12.20 (s, 1H), 8.62 (bs, 1H), 8.32 (s, 1H), 7.89 (s, 1H), 7.51 (m, 2H), 3.90 (s, 3H), 3.40 (m, 2H), 2.84 (t, J=6.4 Hz, 2H). HRMS calculated for C₁₄H₁₃N₃O₃ (MH⁺) 272.1030, found 272.1016. Anal. calculated for C₁₄H₁₃N₃O₃.0.7 TFA.2.0 H₂O C, 47.78; H, 4.60; N, 10.85. Found: C, 47.87; H, 4.70; N, 10.89.

### EXAMPLE 771

This example illustrates the preparation of 4-(4-Oxo-4,5,6,7-tetrahydro-1H-pyrrolo[3,2-c]pyridin-2-yl)pyridine-2-carboxylic acid trifluoroacetate.

A solution of the compound made according to Example 770 (250 mg, 0.65 mmol) in 3.0 mL methanol and 2.0 mL water was treated with 0.98 mL of 2 M KOH and stirred for 2 hours. Condensed to ⅓ vol and added acetonitrile to precipitate a solid. Filtered solid washing with acetonitrile. Dissolved solid in water, added TFA and filtered the resulting precipitate to give the title compound as a brown solid (142 mg, 0.38 mmol, 59%). ¹H NMR (300 MHz, DMSO-d₆) δ 12.18 (s, 1H), 8.56 (s, 1H), 8.29 (s, 1H), 7.86 (s, 1H), 7.11 (s, 2H), 3.40 (m, 2H), 2.84 (m, 2H). Anal. calculated for C₁₃H₁₁N₃O₃.1.8 H₂O C, 53.90; H, 5.08; N, 14.50. Found: C, 53.91; H, 4.79; N, 14.38.

### EXAMPLE 772

This example illustrates the preparation of 4-(4-Oxo-4,5,6,7-tetrahydro-1H-pyrrolo[3,2-c]pyridin-2-yl)-N-phenylpyridine-2-carboxamide trifluoroacetate.

A suspension of the compound made in Example 771 in 4.0 mL tetrahydrofuran was treated with 2.5 mL of thionyl chloride and heated to 65° C. for 3 hours and condensed to dryness. The residue was suspended in 4.0 mL of tetrahydrofuran and treated with aniline (0.04 mL, 0.42 mmol) and 0.08 mL of triethylamine and stirred for 18 hours. The reaction was acidified by adding trifluoroacetic acid, filtered through a syringe filter (0.45 um), purified by rpHPLC and lyophilized to give the title compound as a yellow solid (52 mg, 0.12 mmol, 40%). ¹H NMR (300 MHz, DMSO-d₆) δ 12.25 (s, 1H), 10.61 (s, 1H), 8.61 (d, J=5.2 Hz, 1H), 8.41 (s, 1H), 7.91 (m, 2H), 7.37 (t, J=7.6 Hz, 1H), 7.13 (m, 2H), 3.43 (t, J=6.6 Hz, 2H), 2.85 (t, J=6.8 Hz, 2H). HRMS calculated for C₁₉H₁₆N₄O₂ (MH⁺) 333.1346, found 333.1345. Anal. calculated for C₁₉H₁₆N₄O₂.1.0 TFA.0.6 H₂O C, 55.16; H, 4.01; N, 12.25. Found: C, 55.53; H, 4.41; N, 12.42.

### EXAMPLE 773

This example illustrates the preparation of N-benzyl-4-(4-oxo-4,5,6,7-tetrahydro-1H-pyrrolo[3,2-c]pyridin-2-yl)pyridine-2-carboxamide trifluoroacetate.

A suspension of the compound synthesized according to Example 771 (150 mg, 0.58 mmol), EDCI (123 mg, 0.64 mmol) and 1-hydroxybenzitriazole (86 mg, 0.64 mmol) in 4.0 mL of dimethylformamide was treated with diisopropylethylamine (0.13 mL, 0.76 mmol) followed by benzylamine (0.07 mL, 0.64 mmol) and stirred for 2 hours then heated to 60° C. for 2 hours. The reaction was cooled and acidified with 0.75 mL of TFA, filtered through a syringe filter, purified by rpHPLC and lyophilized to give the title compound as a yellow solid (50 mg, 0.11 mmol, 19%). ¹H NMR (300 MHz, DMSO-d₆) δ 12.20 (s, 1H), 9.32 (t, J=6.3 Hz, 1H), 8.53 (d, J=5.4 Hz, 1H), 8.30 (s, 1H), 7.83 (d, J=5.2 Hz, 1H), 7.37-7.20 (m, 5H), 7.09 (s, 2H), 4.51 (d, J=6.2 Hz, 2H), 3.40 (t, J=6.7 Hz, 2H), 2.83 (t, J=6.7 Hz, 2H). HRMS calculated for C₂₀H₁₈N₄O₂ (MH⁺) 347.1503, found 347.1505. Anal. calculated for C₂₀H₁₈N₄O₂.0.55 TFA.0.60 H₂O C, 60.35; H, 4.74; N, 13.34. Found: C, 60.32; H, 4.69; N, 13.35.

The following Examples were prepared by this method:

| | | Calculated | Found |
|---|---|---|---|
| Example | | Exact Mass | Exact Mass |
| No. | Compound | (m + H) | (m + H) |
| EXAMPLE 774 | N-methyl-4-(4-oxo-4,5,6,7- | 347.1503 | 347.1515 |
| | tetrahydro-1H-pyrrolo[3,2- | | |
| | c]pyridin-2-yl)-N-phenylpyridine- | | |
| | 2-carboxamide trifluoroacetate | | |
| EXAMPLE 775 | 4-(4-oxo-4,5,6,7-tetrahydro-1H- | 334.1299 | 334.1284 |
| | pyrrolo[3,2-c]pyridin-2-yl)-N- | | |
| | pyridin-2-ylpyridine-2-carboxamide | | |
| | trifluoroacetate | | |

### EXAMPLE 776

This example illustrates the preparation of phenyl 4-(4-oxo-4,5,6,7-tetrahydro-1H-pyrrolo[3,2-c]pyridin-2-yl)pyridine-2-carboxylate trifluoroacetate.

4-(4-Oxo-4,5,6,7-tetrahydro-1H-pyrrolo[3,2-c]pyridine-2-yl)pyridine-2-carboxylic acid (0.053 g, 0.206 mmol) and fluoro-N,N,N′N,′-tetramethyl-formamidium hexafluorophosphate (TFFH) (0.060 g, 0.228 mmol) were placed in an oven dried vial under N₂ atm and dissolved in 2.0 mL of dry DMSO. 0.110 mL (0.6314 mmol) of the DIEA was added and all solids dissolved. After stirring at rt for 30 min, powdered sodium phenoxide was added and the reaction mixture was stirred at rt for 1 h. The reaction was complete by analytical HPLC and LC-MS. The crude residue was purified by reverse-phase C18 chromatography with a water/acetonitrile gradient containing 0.1% TFA and lyophilized to afford the title compound as an orange solid (0.0337 g). Calculated exact mass 334.1186; Found 334.1224.

The following compounds were prepared using the above procedure. The purified products were characterized by analytical reverse phase HPLC, LC-MS, ¹H NMR, ¹³ C NMR, and HR-MS.

| | | Calculated | Found |
|---|---|---|---|
| Example | | Exact Mass | Exact Mass |
| No. | Compound | (m + H) | (m + H) |
| EXAMPLE 777 | N-methoxy-N-methyl-4-(4-oxo- | 301.1296 | 301.1293 |
| | 4,5,6,7-tetrahydro-1H-pyrrolo[3,2- | | |
| | c]pyridin-2-yl)pyridine-2- | | |
| | carboxamide trifluoroacetate | | |
| EXAMPLE 778 | S-phenyl 4-(4-oxo-4,5,6,7- | 350.0958 | 350.0917 |
| | tetrahydro-1H-pyrrolo[3,2- | | |
| | c]pyridin-2-yl)pyridine-2- | | |
| | carbothioate trifluoroacetate | | |
| EXAMPLE 779 | 4-[5-fluoro-4-(4-oxo-4,5,6,7- | 442.1674 | 442.1671 |
| | tetrahydro-1H-pyrrolo[3,2- | | |
| | c]pyridin-2-yl)pyridin-2-yl]-N- | | |
| | (pyridin-4-ylmethyl) benzamide | | |
| | trifluoroacetate | | |

### EXAMPLE 780

This example illustrates the preparation of 2-Ketopyridine analog compounds.

To a solution of N-methoxy-N-methyl-4-(4-oxo-4,5,6,7-tetrahydro-1H-pyrrolo[3,2-c]pyridin-2-yl) pyridine-2-carboxamide trifluoroacetate in THF was added a solution of phenyllithium or butyllithium or methylmagnesium bromide or benzylmagnesium bromide in ether or cyclohexane at −78° C. and the reaction was stirred at that temperature for 2 h. The reaction mixture was quenched with saturated NH₄Cl at −78° C. The crude residue was purified by reverse-phase C18 preparative chromatography with a water/acetonitrile gradient containing 0.1% TFA and lyophilized to afford the desired compound.

The following compounds were prepared using the above general procedure. The purified products were characterized by analytical reverse phase HPLC, LC-MS, ¹H NMR, ¹³C NMR, and HR-MS.

| | | Calculated | Found |
|---|---|---|---|
| Example | | Exact Mass | Exact Mass |
| No. | Compound | (m + H) | (m + H) |
| EXAMPLE 781 | 2-(2-benzoylpyridin-4-yl)-1,5,6,7-tetrahydro- | 318.1237 | 318.1214 |
| | 4H-pyrrolo[3,2-c]pyridin-4-one | | |
| | trifluoroacetate | | |
| EXAMPLE 782 | 2-(2-acetylpyridin-4-yl)-1,5,6,7-tetrahydro- | 256.1081 | 256.1051 |
| | 4H-pyrrolo[3,2-c]pyridin-4-one | | |
| | trifluoroacetate | | |
| EXAMPLE 783 | 2-[2-(phenylacetyl)pyridin-4-yl]-1,5,6,7- | 332.1394 | 332.1415 |
| | tetrahydro-4H-pyrrolo[3,2-c]pyridin-4-one | | |
| | trifluoroacetate | | |
| EXAMPLE 784 | 2-{2-[(2-phenyl-1,3-dithian-2- | 436.1148 | 436.1118 |
| | yl)carbonyl]pyridin-4-yl}-1,5,6,7- | | |
| | tetrahydro-4H-pyrrolo[3,2-c]pyridin-4-one | | |
| | trifluoroacetate | | |
| EXAMPLE 785 | 2-(2-pentanoylpyridin-4-yl)-1,5,6,7-tetrahydro- | 298.155 | 298.1557 |
| | 4H-pyrrolo[3,2-c]pyridin-4-one trifluoroacetate | | |

### EXAMPLE 786

This example illustrates the preparation of oximes and hydrazones of 2-Ketopyridine analog compounds.

To a solution of a ketone in methanol was added 2-3 equivalents of hydroxylamine.HCl or hydrazine or phenylhydrazine in methanol and the reaction mixture was stirred at room temperature, or heated under reflux in case of hydroxylamine, overnight. The reaction was monitored to completion by analytical HPLC and LC-MS. The crude residue was purified by reverse-phase C18 preparative chromatography with a water/acetonitrile gradient containing 0.1% TFA to separate the E- and Z-isomers and lyophilized to afford the desired compound.

The following compounds were prepared using the above general procedure. The purified products (E and Zisomers) were characterized by analytical reverse phase HPLC, LC-MS, ¹H NMR, ¹³C NMR, and HR-MS.

| | | Calculated | Found |
|---|---|---|---|
| Example | | Exact Mass | Exact Mass |
| No. | Compound | (m + H) | (m + H) |
| EXAMPLE 787 | 2-{2-[(Z)-(hydroxyimino)(phenyl)methyl]pyridin- | 333.1346 | 333.1366 |
| | 4-yl}-1,5,6,7-tetrahydro-4H- | | |
| | pyrrolo[3,2-c]pyridin-4-one | | |
| | trifluoroacetate | | |
| EXAMPLE 788 | 2-{2-[(E)- | 333.1346 | 333.1352 |
| | (hydroxyimino)(phenyl)methyl]pyridin- | | |
| | 4-yl}-1,5,6,7-tetrahydro-4H- | | |
| | pyrrolo[3,2-c]pyridin-4-one | | |
| | trifluoroacetate | | |
| EXAMPLE 789 | 2-{2-[(E)- | 332.1506 | 332.1509 |
| | hydrazono(phenyl)methyl]pyridin-4- | | |
| | yl}-1,5,6,7-tetrahydro-4H- | | |
| | pyrrolo[3,2-c]pyridin-4-one | | |
| | trifluoroacetate | | |
| EXAMPLE 790 | 2-{2-[(1E)-N,2- | 422.1975 | 422.1939 |
| | diphenylethanehydrazonoyl]pyridin-4- | | |
| | yl}-1,5,6,7-tetrahydro-4H- | | |
| | pyrrolo[3,2-c]pyridin-4-one | | |
| | trifluoroacetate | | |
| EXAMPLE 791 | 2-{2-[(1Z)-N,2- | 422.1975 | 422.1975 |
| | diphenylethanehydrazonoyl]pyridin-4- | | |
| | yl}-1,5,6,7-tetrahydro-4H- | | |
| | pyrrolo[3,2-c]pyridin-4-one | | |
| | trifluoroacetate | | |

### EXAMPLE 792

This example illustrates the preparation of 2-(RR′C═N—NH)— Pyridine analog compounds.

Step 1: A mixture of 2-(2-chloropyridin-4-yl)-1,5,6,7-tetrahydro-4H-pyrrolo[3,2-c]pyridin-4-one (2.00 g, 8.1 mmol) and anhydrous hydrazine (20.00 mL, 640 mmol) was heated under nitrogen at 110° C. for 4 h. The reaction was complete by LCMS, and was concentrated under vacuum without heating to afford a tan solid. This material was diluted with MeOH and acidified with a small volume of concentrated HCl, and then the resulting solid was collected by vacuum filtration. Although this very pure mixture of 2 products could not be easily dissolved in a small volume of H₂O, it could be dissolved in a large volume of H₂O and concentrated under vacuum to <20 mL with very little crystallization of the products. This supersaturated solution was purified by preparative reverse phase chromatography eluted with water to afford 2-(2-Hydrazinopyridin-4-yl)-1,5,6,7-tetrahydro-4H-pyrrolo[3,2-c]pyridin-4-one and 2-(2-Aminoethyl)-5-(2-hydrazinopyridin-4-yl)-1H-pyrrole-3-carbohydrazide. The purified compounds were assayed by analytical reverse phase HPLC, NMR, and MS.

For compound 2-(2-Hydrazinopyridin-4-yl)-1,5,6,7-tetrahydro-4H-pyrrolo[3,2-c]pyridin-4-one: Calculated Exact Mass 243.1120; Found Positive electrospray LC-MS, m/e 244 (M+H⁺).

For compound 2-(2-Aminoethyl)-5-(2-hydrazinopyridin-4-yl)-1H-pyrrole-3-carbohydrazide: Calculated Exact Mass 275.1495; Found Positive electrospray LC-MS, m/e 276 (M+H⁺).

Step 2: To an 0.25M slurry of 2-(2-Hydrazinopyridin-4-yl)-1,5,6,7-tetrahydro-4H-pyrrolo[3,2-c]pyridin-4-one in DMSO or DMF was added 3-5 equivalents of an aldehyde or ketone. The reaction was stirred at room temperature and monitored to completion by RP-HPLC.

Purification was effected by trituration with ether or acetonitrile, or by reverse phase chromatography. The following compounds were prepared with this method.

| | | | Found |
|---|---|---|---|
| Example | | Calculated | Electrospray |
| No. | Compound | Exact Mass | m + H |
| EXAMPLE 793 | benzaldehyde[4-(4-oxo-4,5,6,7- | 331.1433 | 332 |
| | tetrahydro-1H-pyrrolo[3,2-c]pyridin-2- | | |
| | yl)pyridin-2-yl]hydrazone | | |
| EXAMPLE 794 | 2-{2-[2-(1- | 283.1433 | 284 |
| | methylethylidene)hydrazino]pyridin-4-yl}- | | |
| | 1,5,6,7-tetrahydro-4H-pyrrolo[3,2- | | |
| | c]pyridin-4-one | | |
| EXAMPLE 795 | 2-[2-(2-cyclohexylidenehydrazino)pyridin- | 323.1746 | 324 |
| | 4-yl]-1,5,6,7-tetrahydro-4H-pyrrolo[3,2- | | |
| | c]pyridin-4-one | | |
| EXAMPLE 796 | 2-{2-[(2E)-2-(1- | 345.1590 | 346.0 |
| | phenylethylidene)hydrazino]pyridin-4-yl}- | | |
| | 1,5,6,7-tetrahydro-4H-pyrrolo[3,2- | | |
| | c]pyridin-4-one | | |
| EXAMPLE 797 | 2-(2-{2-[bis(4- | 443.1558 | 444 |
| | fluorophenyl)methylene]hydrazino}pyridin- | | |
| | 4-yl)-1,5,6,7-tetrahydro-4H-pyrrolo[3,2- | | |
| | c]pyridin-4-one | | |
| EXAMPLE 798 | (1E)-butanal[4-(4-oxo-4,5,6,7- | 297.1590 | 298 |
| | tetrahydro-1H-pyrrolo[3,2-c]pyridin-2- | | |
| | yl)pyridin-2-yl]hydrazone | | |
| EXAMPLE 799 | 2-morpholin-4-ylbenzaldehyde[4-(4-oxo- | 416.1961 | 417.1 |
| | 4,5,6,7-tetrahydro-1H-pyrrolo[3,2- | | |
| | c]pyridin-2-yl)pyridin-2-yl]hydrazone | | |
| EXAMPLE 800 | 4-{[2-(pyrrolidin-1-ylmethyl)pyrrolidin- | 511.2696 | 512.4 |
| | 1-yl]carbonyl}benzaldehyde[4-(4-oxo- | | |
| | 4,5,6,7-tetrahydro-1H-pyrrolo[3,2- | | |
| | c]pyridin-2-yl)pyridin-2-yl]hydrazone | | |
| | bis(trifluoroacetate) | | |

### EXAMPLE 801

This example illustrates the preparation of 2-[2-(1-Methylhydrazino) pyridin-4-yl]-1,5,6,7-tetrahydro-4H-pyrrolo[3,2-c]pyridin-4-one.

A mixture of 2-(2-chloropyridin-4-yl)-1,5,6,7-tetrahydro-4H-pyrrolo[3,2-c]pyridin-4-one (2.00 g, 8.1 mmol) and methylhydrazine (20.00 mL, 376 mmol) was refluxed under nitrogen until complete by LCMS after 7 hours. The reaction was diluted with 40 mL of H₂O and allowed to stand at room temperature until crystallization began, then was placed in the refrigerator overnight. The solid was collected by filtration, washed with H₂O, and vacuum dried to give 2.15 g of 2-[2-(1-Methylhydrazino)pyridin-4-yl]-1,5,6,7-tetrahydro-4H-pyrrolo[3,2-c]pyridin-4-one as a tan solid that was characterized by analytical reverse phase HPLC, NMR, and MS. Calculated Exact Mass 257.1277; Found Positive Electrospray LC-MS, m/e 258.1 (M+H⁺).

### EXAMPLE 802

This example illustrates the preparation of 2-(N-Methylhydrazono) Pyridine analog compounds.

To a 0.25-1.0M slurry of 2-[2-(1-Methylhydrazino)pyridin-4-yl]-1,5,6,7-tetrahydro-4H-pyrrolo[3,2-c]pyridin-4-one in DMF was added 1.1-5.0 equivalents of an aldehyde or ketone. The reaction was stirred at room temperature and monitored to completion by RP-HPLC. Purification was effected by trituration with ether or acetonitrile, or by reverse phase chromatography. The following compounds were prepared with this method.

| | | | Found |
|---|---|---|---|
| Example | | Calculated | Electrospray |
| No. | Compound | Exact Mass | m + H |
| EXAMPLE 803 | benzaldehyde methyl[4-(4-oxo-4,5,6,7- | 345.1590 | 346.0 |
| | tetrahydro-1H-pyrrolo[3,2-c]pyridin-2- | | |
| | yl)pyridin-2-yl]hydrazone | | |
| EXAMPLE 804 | 2-{2-[1-methyl-2-(1- | 297.1590 | 298.0 |
| | methylethylidene)hydrazino]pyridin-4-yl}- | | |
| | 1,5,6,7-tetrahydro-4H-pyrrolo[3,2- | | |
| | c]pyridin-4-one | | |
| EXAMPLE 805 | 2-morpholin-4-ylbenzaldehyde methyl[4-(4- | 430.2117 | 431.1 |
| | oxo-4,5,6,7-tetrahydro-1H-pyrrolo[3,2- | | |
| | c]pyridin-2-yl)pyridin-2-yl]hydrazone | | |
| EXAMPLE 806 | 4-((E)-{methyl[4-(4-oxo-4,5,6,7- | 474.2016 | 475.2 |
| | tetrahydro-1H-pyrrolo[3,2-c]pyridin-2- | | |
| | yl)pyridin-2-yl]hydrazono}methyl)-3- | | |
| | morpholin-4-ylbenzoic acid | | |
| EXAMPLE 807 | 4-(morpholin-4-ylcarbonyl)benzaldehyde | 458.2066 | 459.3 |
| | methyl[4-(4-oxo-4,5,6,7-tetrahydro-1H- | | |
| | pyrrolo[3,2-c]pyridin-2-yl)pyridin-2- | | |
| | yl]hydrazone trifluoroacetate | | |
| EXAMPLE 808 | 4-acetylbenzaldehyde methyl[4-(4-oxo- | 387.1695 | 388.0 |
| | 4,5,6,7-tetrahydro-1H-pyrrolo[3,2- | | |
| | c]pyridin-2-yl)pyridin-2-yl]hydrazone | | |
| EXAMPLE 809 | methyl 4-((E)-{methyl[4-(4-oxo-4,5,6,7- | 403.1644 | 404.1 |
| | tetrahydro-1H-pyrrolo[3,2-c]pyridin-2- | | |
| | yl)pyridin-2-yl]hydrazono}methyl)benzoate | | |
| EXAMPLE 810 | 4-hydroxybenzaldehydemethyl[4-(4-oxo- | 361.1539 | 362.1 |
| | 4,5,6,7-tetrahydro-1H-pyrrolo[3,2- | | |
| | c]pyridin-2-yl)pyridin-2-yl]hydrazone | | |
| EXAMPLE 811 | terephthalaldehydemethyl[4-(4-oxo- | 373.1539 | 374.1 |
| | 4,5,6,7-tetrahydro-1H-pyrrolo[3,2- | | |
| | c]pyridin-2-yl)pyridin-2-yl]hydrazone | | |
| | trifluoroacetate | | |
| EXAMPLE 812 | N-[4-((E)-{2-methyl-2-[4-(4-oxo-4,5,6,7- | 402.1804 | 403.0 |
| | tetrahydro-1H-pyrrolo[3,2-c]pyridin-2- | | |
| | yl)pyridin-2- | | |
| | yl]hydrazono}methyl)phenyl]acetamide | | |
| EXAMPLE 813 | 4-methoxybenzaldehyde methyl[4-(4-oxo- | 375.1695 | 376.0 |
| | 4,5,6,7-tetrahydro-1H-pyrrolo[3,2- | | |
| | c]pyridin-2-yl)pyridin-2-yl]hydrazone | | |
| EXAMPLE 814 | 4-(dimethylamino)benzaldehyde methyl[4- | 388.2012 | 389.1 |
| | (4-oxo-4,5,6,7-tetrahydro-1H-pyrrolo[3,2- | | |
| | c]pyridin-2-yl)pyridin-2-yl]hydrazone | | |
| EXAMPLE 815 | 4-(methylsulfonyl)benzaldehyde methyl[4- | 423.1365 | 424.0 |
| | (4-oxo-4,5,6,7-tetrahydro-1H-pyrrolo[3,2- | | |
| | c]pyridin-2-yl)pyridin-2-yl]hydrazone | | |
| EXAMPLE 816 | 4-((E)-{2-methyl-2-[4-(4-oxo-4,5,6,7- | 403.1644 | 404.1 |
| | tetrahydro-1H-pyrrolo[3,2-c]pyridin-2- | | |
| | yl)pyridin-2-yl]hydrazono}methyl)phenyl | | |
| | acetate | | |

### EXAMPLE 817

This example illustrates the preparation of 2-Heterosubstituted Pyridine analog compounds.

Step 1: A slurry of 2-(2-chloropyridin-4-yl)-1,5,6,7-tetrahydro-4H-pyrrolo[3,2-c]pyridin-4-one (2.25 g, 9.1 mmol) and allyl bromide (15.00 mL, 173 mmol) in DMF (15.00 mL) was heated under nitrogen at 70° C. and followed to completion over 2 days by HPLC. The resulting bright yellow slurry was filtered and vacuum dried to give 3.47 g of 1-allyl-2-chloro-4-(4-oxo-4,5,6,7-tetrahydro-1H-pyrrolo[3,2-c]pyridin-2-yl)pyridinium bromide as a yellow solid, which was characterized by analytical reverse phase HPLC, NMR, and MS. Calculated Exact Mass 288.0904 (for cation); Found Positive Electrospray LC-MS, m/e 288.0 (M⁺).

Step 2: To a 0.5M mixture of the product from step 1 (1.0 equiv) in DMF was added 1.5 equiv of either the sodium or potassium salt of a phenol or phthalimide, or the DIEA salt of an oxime. The reaction was monitored to completion by HPLC, then a 4M solution of 1:1 DIEA/HCO₂H (5.0 equiv) in DMF was added, followed immediately by the addition of 5 mol% Pd(PPh₃)₄. When deallylation was complete by HPLC and LCMS, the reaction was diluted with 1:1 DMF/H₂O, filtered, and purified by reverse-phase chromatography. The resulting 2-heterosubstituted pyridine derivatives were characterized by analytical reverse phase HPLC, NMR, and MS. The following compounds were prepared with this method.

| | | | Found |
|---|---|---|---|
| Example | | Calculated | Electrospray |
| No. | Compound | Exact Mass | m + H |
| EXAMPLE 818 | 2-(2-phenoxypyridin-4-yl)-1,5,6,7- | 305.1164 | 306 |
| | tetrahydro-4H-pyrrolo[3,2-c]pyridin-4-one | | |
| EXAMPLE 819 | 2-[2-({[(1E)-1- | 346.1430 | 347.2 |
| | phenylethylidene]amino}oxy)pyridin-4-yl]- | | |
| | 1,5,6,7-tetrahydro-4H-pyrrolo[3,2- | | |
| | c]pyridin-4-one trifluoroacetate | | |
| EXAMPLE 820 | (2Z)-({[4-(4-oxo-4,5,6,7-tetrahydro-1H- | 357.1226 | 358.2 |
| | pyrrolo[3,2-c]pyridin-2-yl)pyridin-2- | | |
| | yl]oxy}imino)(phenyl)acetonitrile | | |
| | trifluoroacetate | | |
| EXAMPLE 821 | 2-[4-(4-oxo-4,5,6,7-tetrahydro-1H- | 358.1066 | 359.1 |
| | pyrrolo[3,2-c]pyridin-2-yl)pyridin-2-yl]- | | |
| | 1H-isoindole-1,3(2H)-dione | | |
| | trifluoroacetate | | |

### EXAMPLE 822

This example illustrates the preparation of 2-[2-(Phenylthio)pyridin-4-yl]-1,5,6,7-tetrahydro-4H-pyrrolo[3,2-c]pyridin-4-one trifluoroacetate.

Step 1: MeOSO₂CF₃ (2.30 mL, 20.3 mmol) was added to a slurry of 2-(2-chloropyridin-4-yl)-1,5,6,7-tetrahydro-4H-pyrrolo[3,2-c]pyridin-4-one (5.00 g, 20.2 mmol) in CHCl₃ (50 mL) with no exotherm. The reaction was complete by HPLC after stirring overnight at room temperature. A small amount of methanol was then added to the resulting bright yellow slurry, and the solid was collected by filtration and vacuum dried to give 5.38g of 1-methyl-4-(4-oxo-4,5,6,7-tetrahydro-1H-pyrrolo[3,2-c]pyridin-2-yl)-2-(phenylthio)pyridinium trifluoromethanesulfonate as a yellow solid that was analyzed by analytical reverse phase HPLC, H-NMR, F-NMR, and MS. Calculated Exact Mass 262.0747 (for cation); Found Positive Electrospray LC-MS, m/e 262.0 (M⁺).

Step 2: Sodium thiophenoxide (482 mg, 3.65 mmol) was added to a slurry of the product from step 1 (300 mg, 0.73 mmol) in DMF (1.50 mL). The solution that formed in a few minutes was heated to 100° C. and monitored by LCMS for complete demethylation over 4 h. The resulting mixture was filtered and purified by reverse phase chromatography to give 114 mg of 2-[2-(Phenylthio)pyridin-4-yl]-1,5,6,7-tetrahydro-4H-pyrrolo[3,2-c]pyridin-4-one trifluoroacetate as a light green solid that was characterized by analytical reverse phase HPLC, NMR, and MS. Calculated Exact Mass 321.0936; Found Positive Electrospray LC-MS, m/e 322.0 (M+H⁺).

### EXAMPLE 823

This example illustrates the preparation of 2-Acylamino Pyridine analog compounds.

Step 1: Bromine (1.6 mL, 31.2 mmol) was added to a solution of 2-amino-4-acetylpyridine hydrochloride (1983JHetChem533, 5.2 g, 30.1 mmol) and 30% HBr in AcOH (6.5 mL, 32.6 mmol) in AcOH (51 mL). Within 10 minutes, a thick, creamy yellow slurry formed that was diluted with additional AcOH (50 mL). After stirring at room temperature for 3h, the solid was collected by filtration and washed with ether. Vacuum drying gave 8.0 g of 1-(2-aminopyridin-4-yl)-2-bromoethanone hydrobromide, which was characterized by analytical reverse phase HPLC, NMR, and MS. Calculated Exact Mass 213.9742; Found Positive Electrospray LC-MS, m/e 215.0 (M+H⁺).

Step 2: A solution of the product from step 1 (8.75 g, 29.6 mmol), 2,4-diketopiperidine (3.5 g, 31.0 mmol), and NH₄OAc (9.92 g, 128.8 mmol) in ethanol (88 mL) was stirred at room temperature for 3 h. After removal of the solvent under vacuum, the product was slurried in CH₃CN (100 mL), collected by filtration, and vacuum dried to give 14.47 g of a mixture of 2-(2-aminopyridin-4-yl)-1,5,6,7-tetrahydro-4H-pyrrolo[3,2-c]pyridin-4-one and NH₄OAc. 1.0 g of this material was purified by reverse phase chromatography to give 0.3 g of the free base of 2-(2-aminopyridin-4-yl)-1,5,6,7-tetrahydro-4H-pyrrolo[3,2-c]pyridin-4-one, which was characterized by analytical reverse phase HPLC and NMR.

Step 3: EDC (1 equivalent) was added to an ice water cooled, 0.2M solution of a carboxylic acid (2 equivalents) in CH₂Cl₂. The mixture was stirred at 0° C. for 2 h, then was washed sequentially with cold 1 M HCl, saturated NaHCO₃, and then brine. The organic solution was dried (Na₂SO₄) and concentrated to give the symmetric anhydride. The following compounds were prepared by this method.

| | | | Found |
|---|---|---|---|
| Example | | Calculated | Electrospray |
| No. | Compound | Exact Mass | m + H |
| EXAMPLE 824 | (2E)-3-phenylacrylic | 278.09 | 279 |
| | anhydride | | |
| EXAMPLE 825 | (2E)-2-methyl-3- | 306.13 | 307 |
| | phenylacrylic anhydride | | |
| EXAMPLE 826 | (2E)-3-methyl-3- | 306.13 | 307 |
| | phenylacrylic anhydride | | |
| EXAMPLE 827 | (2Z)-2-fluoro-3- | 314.08 | 315 |
| | phenylacrylic anhydride | | |
| EXAMPLE 828 | 3-phenylpropanoic | 282.13 | 283 |
| | anhydride | | |
| EXAMPLE 829 | 3-phenylprop-2- | 274.06 | 275 |
| | ynoic anhydride | | |

Step 4: A 0.67M mixture of the product of step 2 in the preparation of 2-Acylamino Pyridine analog compounds according to Example 823 (1 equivalent), a symmetric anhydride (1.1 equivalents), and 4-DMAP (0.8 equivalent) in DMF was heated to 80° C. for 3 h. The resulting mixture was filtered and purified by reverse phase HPLC to give the 2-(2-amidopyridin-4-yl)-1,5,6,7-tetrahydro-4H-pyrrolo[3,2-c]pyridin-4-one as a solid, which was characterized by analytical reverse phase HPLC, NMR, and MS. The following compounds were prepared by this method.

| | | | Found |
|---|---|---|---|
| Example | | Calculated | Electrospray |
| No. | Compound | Exact Mass | m + H |
| EXAMPLE 830 | tert-butyl 4-(4-oxo-4,5,6,7- | 328.37 | 329 |
| | tetrahydro-1H-pyrrolo[3,2- | | |
| | c]pyridin-2-yl)pyridin-2- | | |
| | ylcarbamate | | |
| EXAMPLE 831 | (2E)-N-[4-(4-oxo-4,5,6,7- | 358.40 | 356 |
| | tetrahydro-1H-pyrrolo[3,2- | | |
| | c]pyridin-2-yl)pyridin-2- | | |
| | yl]-3-phenylacrylamide | | |
| EXAMPLE 832 | N-[4-(4-oxo-4,5,6,7- | 270.29 | 271 |
| | tetrahydro-1H-pyrrolo[3,2- | | |
| | c]pyridin-2-yl)pyridin-2- | | |
| | yl]acetamide | | |
| EXAMPLE 833 | (2Z)-2-fluoro-N-[4-(4-oxo- | 376.39 | 377 |
| | 4,5,6,7-tetrahydro-1H- | | |
| | pyrrolo[3,2-c]pyridin-2- | | |
| | yl)pyridin-2-yl]-3- | | |
| | phenylacrylamide | | |
| EXAMPLE 834 | (2E)-N-[4-(4-oxo-4,5,6,7- | 372.43 | 373 |
| | tetrahydro-1H-pyrrolo[3,2- | | |
| | c]pyridin-2-yl)pyridin-2- | | |
| | yl]-3-phenylbut-2-enamide | | |
| EXAMPLE 835 | (2E)-2-methyl-N-[4-(4-oxo- | 372.43 | 373 |
| | 4,5,6,7-tetrahydro-1H- | | |
| | pyrrolo[3,2-c]pyridin-2- | | |
| | yl)pyridin-2-yl]-3- | | |
| | phenylacrylamide | | |
| EXAMPLE 836 | 2-methyl-N-[4-(4-oxo- | 484.52 | 485 |
| | 4,5,6,7-tetrahydro-1H- | | |
| | pyrrolo[3,2-c]pyridin-2- | | |
| | yl)pyridin-2-yl]-bis(3- | | |
| | phenylpropynoyl)amide | | |
| EXAMPLE 837 | N-[4-(4-oxo-4,5,6,7- | 360.42 | 361 |
| | tetrahydro-1H-pyrrolo[3,2- | | |
| | c]pyridin-2-yl)pyridin-2- | | |
| | yl]-3-phenylpropanamide | | |

### EXAMPLE 838

This example illustrates the preparation of 2-Oxo-N-[4-(4-oxo-4,5,6,7-tetrahydro-1H-pyrrolo[3,2-c]pyridin-2-yl)pyridin-2-yl]-2-phenylacetamide trifluoroacetate and N,N-Dimethyl-N′-[4-(4-oxo-4,5,6,7-tetrahydro-1H-pyrrolo[3,2-c]pyridin-2-yl)pyridin-2-yl]imidoformamide.

Step 1: A solution of benzoylformic acid (0.75 g, 5 mmol) in neat thionyl chloride (1.82 mL, 25 mmol) was heated to 75° C. for 3 h. Excess thionyl chloride was removed under vacuum to give a light yellow oil that was used without further purification.

Step 2: The benzoylformyl chloride (74 mg, 0.52 mmol) prepared in step 1 was added to a dry-ice/acetonitrile cooled solution of the product of step 2 in the preparation of 2-Acylamino Pyridine analog compounds synthesized according to Example 823 (100 mg, 0.44 mmol) and DIEA (76 μL, 0.44 mmol) in DMF (1 mL). The light yellow solution turned dark brown immediately, and was warmed to room temperature, filtered, purified by reverse phase HPLC and the products characterized by analytical reverse phase HPLC, NMR, and MS. 2-Oxo-N-[4-(4-oxo-4,5,6,7-tetrahydro-1H-pyrrolo[3,2-c]pyridin-2-yl)pyridin-2-yl]-2-phenylacetamide trifluoroacetate (3 mg): Calculated Exact Mass 360.1222; Found Positive Electrospray LC-MS, m/e 361 (M+H⁺). N,N-Dimethyl-N′-[4-(4-oxo-4,5,6,7-tetrahydro-1H-pyrrolo[3,2-c]pyridin-2-yl)pyridin-2-yl]imidoformamide (1 mg): Calculated Exact Mass 283.1433; Found Positive Electrospray LC-MS, m/e 284 (M+H⁺).

### EXAMPLE 840

This example illustrates the preparation of 2-[2-(Methylamino)pyridin-4-yl]-1,5,6,7-tetrahydro-4H-pyrrolo[3,2-c]pyridin-4-one.

A slurry of 2-[2-(1-methylhydrazino)pyridin-4-yl]-1,5,6,7-tetrahydro-4H-pyrrolo[3,2-c]pyridin-4-one (2.1 g, 8.1 mmol) in EtOH (50 mL) was hydrogenated over 10% Pd/C at 60 psi H₂ and monitored to completion by LCMS. To separate the product from the catalyst, the solvent was removed under vacuum, and the product was then dissolved in DMF, filtered, and concentrated under under vacuum to give 2.2 g of product as a white solid, which was characterized by analytical reverse phase HPLC, NMR, and MS. Calculated exact mass 242.1168; Found Positive Electrospray LC-MS, m/e 243 (M+H⁺).

### EXAMPLE 841

This example illustrates the preparation of 2-[2-(pyridin-2-ylamino)pyridin-4-yl]-1,5,6,7-tetrahydro-4H-pyrrolo[3,2-c]pyridin-4-one trifluoroacetate.

A suspension of 2-(2-chloropyridin-4-yl)-1,5,6,7-tetrahydro-4H-pyrrolo[3,2-c]pyridin-4-one (300 mg, 1.2 mmol) and 2-amino pyridine (228 mg, 2.4 mmol) in 10.0 mL of dimethylformamide was treated with tris(dibenzylideneacetone)dipalladium (0) (55 mg, 0.06 mmol), racemic-2,2′-bis(diphenylphosphino)-1,1′-binaphthyl (112 mg, 0.18 mmol) and sodium tert-butoxide (345 mg, 3.6 mmol) and heated to 100 degrees celcius for 18 hours. The reaction was cooled to room temperature, acidified with trifluoroacetic acid, filtered through a syringe filter (0.45 μm), purified by rpHPLC and lyophilized to give the title compound as a yellow solid (115 mg, 0.2 mmol, 18%). ¹H NMR (400 MHz, DMSO-d₆) δ 12.34 (s, 1H), 11.58 (s, 1H), 8.37 (d, J=5.2 Hz, 1H), 8.22 (d, J=6.4 Hz, 1H), 7.97 (t, J=8.7 Hz, 1H), 7.47 (d, J=5.2 Hz, 1H), 7.38 (s, 1H), 7.28 (d, J=8.7 Hz, 1H), 7.21-7.17 (m, 2H), 7.13 (s, 1H), 3.41 (t, J=6.8 Hz, 2H), 2.87 (t, J=6.5 Hz, 2H). HRMS calculated for C₁₇H₁₅N₅O (MH⁺) 306.1349, found 306.1329. Anal. calculated for C₁₇H₁₅N₅0 1.4 TFA 1.4 H₂O C, 48.42; H, 3.96; N, 14.26. Found: C, 48.45; H, 4.05; N, 14.22.

The following Example was prepared in the same manner:

| | | Calculated | Found |
|---|---|---|---|
| Example | | Exact Mass | Exact Mass |
| No. | Compound | (m + H) | (m+H) |
| EXAMPLE 842 | 2-[2-(pyridin-3-ylamino)pyridin- | 306.1349 | 306.1326 |
| | 4-yl]-1,5,6,7-tetrahydro-4H- | | |
| | pyrrolo[3,2-c]pyridin-4-one | | |
| | trifluoroacetate | | |

### EXAMPLE 843

This example illustrates the preparation of 2-(2-anilinopyridin-4-yl)-1,5,6,7-tetrahydro-4H-pyrrolo[3,2-c]pyridin-4-one trifluoroacetate.

A suspension of 2-(2-chloropyridin-4-yl)-1,5,6,7-tetrahydro-4H-pyrrolo[3,2-c]pyridin-4-one (200mg, 0.8 mmol) in aniline (2.0 mL) was purged with nitrogen (g) 3×. The reaction was then heated to 180 deg C. for 4 hrs, then cooled to room temperature. DMF (2.0 mL) was added to the reaction mixture. The crude mixture was purified by reverse-phase high pressure chromatography (acetonitrile/water/0.05% trifluoroacetic acid), and lyophilized to give the title compound as a white solid (150 mg, 0.28 mmol, 35%) ¹H NMR (300 MHz, CD₃OD) δ 7.80 (d, J=7.2 Hz, 1H), 7.55 (m, 2H), 7.41 (m, 3H), 7.30 (m, 2H), 7.25 (s, 1H), 3.59 (t, J=6.9 Hz, 2H), 2.97 (t, J=6.9 Hz, 2H). HRMS calculated for C₁₈H₁₆N₄O (MH⁺) 305.1397, found 305.1400.

### EXAMPLE 844

This example illustrates the preparation of 2-[2-(1H-benzimidazol-2-ylamino)pyridin-4-yl]-1,5,6,7-tetrahydro-4H-pyrrolo[3,2-c]pyridin-4-one trifluoroacetate.

A mixture of 2-(2-chloropyridin-4-yl)-1,5,6,7-tetrahydro-4H-pyrrolo[3,2-c]pyridin-4-one (200 mg, 0.8 mmol) and 2-aminobenzimidazole (1.0 g, 7.5 mmol) was purged with nitrogen (g) 3×. The reaction was then heated to 230 deg C. for 4 hrs, then cooled to room temperature. DMF (5.0 mL) was added to the reaction mixture. The crude mixture was purified by reverse-phase high pressure chromatography (acetonitrile/water/0.05% trifluoroacetic acid), and lyophilized to give the title compound as a white solid. ¹H NMR (500 MHz, DMSO-d₆) δ 12.12 (s, 1H), 8.51 (d, J=4.6 Hz, 1H), 7.86 (s, 1H), 7.68 (d, J=1.5 Hz, 1H), 7.34 (d, J=2.2 Hz, 1H), 7.25 (d, J=2.4 Hz, 1H), 7.14 (s, 1H), 7.07 (m, 1H), 6.97 (m, 1H), 6.70 (m, 2H), 3.41 (t, J=6.85 Hz, 2H), 2.84 (t, J=6.85 Hz, 2H). HRMS calculated for C₁₉H₁₆N₆O (MH⁺) 345.1458, found 345.1458.

### EXAMPLE 845

This example illustrates the preparation of 2-{2-[(4-morpholin-4-ylphenyl)amino]pyridin-4-yl}-1,5,6,7-tetrahydro-4H-pyrrolo[3,2-c]pyridin-4-one.

A mixture of 2-(2-chloropyridin-4-yl)-1,5,6,7-tetrahydro-4H-pyrrolo[3,2-c]pyridin-4-one (200 mg, 0.8 mmol) and 4-morpholinoaniline (1.0 g, 5.6 mmol) was purged with nitrogen (g) 3×. The reaction was then heated to 180 deg C. for 4 hrs, then cooled to room temperature. DMF (5.0 mL) was added to the reaction mixture. The crude mixture was purified by reverse-phase high pressure chromatography (acetonitrile/water/0.05% trifluoroacetic acid), and lyophilized to give the title compound as a white solid. HRMS calculated for C₂₂H₂₃N₅O₂ (MH⁺) 390.1925, found 390.1934.

### EXAMPLE 846

This example illustrates the preparation of 2-{2-[(5-phenyl-1H-pyrazol-3-yl)amino]pyridin-4-yl}-1,5,6,7-tetrahydro-4H-pyrrolo[3,2-c]pyridin-4-one.

A mixture of 2-(2-chloropyridin-4-yl)-1,5,6,7-tetrahydro-4H-pyrrolo[3,2-c]pyridin-4-one (200 mg, 0.8 mmol) and 3-amino-5-phenylpyrazole (1.0 g, 6.28 mmol) was purged with nitrogen (g) 3×. The reaction was then heated to 180 deg C. for 4 hrs, then cooled to room temperature. DMF (5.0 mL) was added to the reaction mixture. The crude mixture was purified by reverse-phase high pressure chromatography (acetonitrile/water/0.05% trifluoroacetic acid), and lyophilized to give the title compound as a white solid. HRMS calculated for C₂₁H₁₈N₆O (MH⁺) 371.1615, found 371.1616.

### EXAMPLE 847

This example illustrates the preparation of 2-{2-[(3-fluorophenyl)amino]pyridin-4-yl}-1,5,6,7-tetrahydro-4H-pyrrolo[3,2-c]pyridin-4-one trifluoroacetate.

A mixture of 2-(2-chloropyridin-4-yl)-1,5,6,7-tetrahydro-4H-pyrrolo[3,2-c]pyridin-4-one (200 mg, 0.8 mmol) and 3-floroaniline (1.0 g, 8.99 mmol) was purged with nitrogen (g) 3×. The reaction was then heated to 180 deg C. for 4 hrs, then cooled to room temperature. DMF (5.0 mL) was added to the reaction mixture. The crude mixture was purified by reverse-phase high pressure chromatography (acetonitrile/water/0.05% trifluoroacetic acid), and lyophilized to give the title compound as a white solid. ¹H NMR (300 MHz, CD₃OD) δ 8.09 (d, J=6.3 Hz, 1H), 8.50 (m, 1H), 7.20 (m, 2H), 7.00 (m, 2H), 6.94 (s, 1H), 6.62 (m, 1H), 3.61 (t, J=6.85 Hz, 2H), 2.97 (t, J=6.85 Hz, 2H). HRMS calculated for C₁8H₁₅FN₄O (MH⁺) 323.1303, found 323.1283.

### EXAMPLE 848

This example illustrates the preparation of 2-{2-[(5-thien-2-yl-1H-pyrazol-3-yl)amino]pyridin-4-yl}-1,5,6,7-tetrahydro-4H-pyrrolo[3,2-c]pyridin-4-one.

A mixture of 2-(2-chloropyridin-4-yl)-1,5,6,7-tetrahydro-4H-pyrrolo[3,2-c]pyridin-4-one (200 mg, 0.8 mmol) and 5-amino-3-(2-thienyl) pyrazole (1.0 g, 6.06 mmol) was purged with nitrogen (g) 3×. The reaction was then heated to 180 deg C. for 4 hrs, then cooled to room temperature. DMF (5.0 mL) was added to the reaction mixture. The crude mixture was purified by reverse-phase high pressure chromatography (acetonitrile/water/0.05% trifluoroacetic acid), and lyophilized to give the title compound as a white solid. ¹H NMR (300 MHz, CD₃OD) δ 8.18 (d, J=6.3 Hz, 1H), 7.40 (m, 2H), 7.35 (m, 2H), 7.10 (m, 2H), 6.94 (s, 1H), 3.61 (t, J=6.85 Hz, 2H), 2.97 (t, J=6.85 Hz, 2H). HRMS calculated for C₁l₉H₆N₆OS (MH⁺) 377.1179, found 377.1187.

### EXAMPLE 849

This example illustrates the preparation of N-[4-(4-oxo-4,5,6,7-tetrahydro-1H-pyrrolo[3,2-c]pyridin-2-yl)pyridin-2-yl]benzamide.

A mixture of 2-(2-chloropyridin-4-yl)-1,5,6,7-tetrahydro-4H-pyrrolo[3,2-c]pyridin-4-one (200 mg, 0.8 mmol) and benzamide (1.0 g, 8.25 mmol) was purged with nitrogen (g) 3×. The reaction was then heated to 180 deg C. for 4 hrs, then cooled to room temperature. DMF (5.0 mL) was added to the reaction mixture. The crude mixture was purified by reverse-phase high pressure chromatography (acetonitrile/water/0.05% trifluoroacetic acid), and lyophilized to give the title compound as a white solid. ¹H NMR (400 MHz, CD₃OD) δ 8.35 (s, 1H), 8.27 (d, J=5.2 Hz, 1H), 7.99 (m, 2H), 7.35 (d, J=5.2 Hz, 1H), 7.32 (s, 1H), 7.17 (d, J=6.8 Hz, 1H), 7.04 (s, 1H), 6.66 (d, J=7.2 Hz, 1H), 3.61 (t, J=6.85 Hz, 2H), 2.97 (t, J=6.85 Hz, 2H). Positive electrospray LC-MS, m/e 333 (M+H⁺).

### EXAMPLE 850

This example illustrates the preparation of N-[4-(4-oxo-4,5,6,7-tetrahydro-1H-pyrrolo[3,2-c]pyridin-2-yl)pyridin-2-yl]-N′-phenylurea.

A mixture of 2-(2-chloropyridin-4-yl)-1,5,6,7-tetrahydro-4H-pyrrolo[3,2-c]pyridin-4-one (200 mg, 0.8 mmol) and phenyl urea (1.0 g, 7.34 mmol) was purged with nitrogen (g) 3×. The reaction was then heated to 180 deg C. for 4 hrs, then cooled to room temperature. DMF (5.0 mL) was added to the reaction mixture. The crude mixture was purified by reverse-phase high pressure chromatography (acetonitrile/water/0.05% trifluoroacetic acid), and lyophilized to give the title compound as a white solid. Positive electrospray LC-MS, m/e 348 (M+H⁺).

### EXAMPLE 851

This example illustrates the preparation of 2-[2-(hydroxymethyl)pyridin-4-yl]-1,5,6,7-tetrahydro-4H-pyrrolo[3,2-c]pyridin-4-one trifluoroacetate.

A suspension of methyl 4-(4-oxo-4,5,6,7-tetrahydro-1H-pyrrolo[3,2-c]pyridin-2-yl)pyridine-2-carboxylate (0.5 g, 1.84 mmol), and sodium borohydride (0.56 g, 14.80 mmol) in methanol (30 mL) was purged with nitrogen (g) 3×. The reaction mixture was stirred at RT for 3 hrs. The solution was concentrated to one half the volume and the pH adjusted to 5 with 1 N hydrogen chloride. The solution was condensed and purified by reverse-phase high pressure chromatography (acetonitrile/water/0.05% trifluoroacetic acid), and lyophilized to give the title compound as a white solid (0.27 g, 60.0% yield): ¹H NMR (300 MHz, CD₃OD) δ 8.5 (d, J=7.2 Hz, 1H), 8.10 (s, 1H), 7.95 (d, J=7.2 Hz, 1H), 7.50 (s, 1H), 4.95 (s, 2H), 3.59 (t, J=6.9 Hz, 2H), 3.02 (t, J=6.9 Hz, 2H). Positive electrospray LC-MS, m/e 244 (M+H⁺).

### EXAMPLE 852

This example illustrates the preparation of 4-(2-hydrazinophenyl)morpholine.

To a suspension of 2-(4-morpholino)aniline (368 mg, 2.0 mmol) in concentrated hydrochloric acid (3 mL) and water (1.5 mL) mixture at −10 deg C. was added dropwise a solution of sodium nitrite (139.0 mg, 2.0 mmol) in water (4.0 mL). After stirring for 2 hours on ice bath, the excess nitrous acid salts were destroyed by the addition of urea (2.0 mmol). The reaction mixture was then treated with tin chloride (1.34 g, 6.02 mmol) dissolved in a mixture of concentrated hydrochloric acid (1.5 mL) and water (22.0 mL). The reaction mixture was stirred for another two hours on ice bath. Saturated sodium sulfide aqueous solution (10 mL) was added to the solution. The resulting yellow solution was concentrated, and basified with 1N sodium hydroxide. The reaction mixture was extracted three times with dichloromethane. The organic extracts were dried over sodium sulfate, filtered and evaporated to give the product. Positive electrospray LC-MS, m/e 194 (M+H⁺).

### EXAMPLE 853

This example illustrates the preparation of methyl 4-(1-methylhydrazino)benzoate.

Step 1: Preparation of methyl 4-[methyl(nitroso)amino] benzoate.

To a suspension of methyl-4-methylamino benzoate (3.3 g, 19.9 mmol) in concentrated hydrochloric acid (33 mL) and water (16 mL) mixture at −10 deg C. was added dropwise a solution of sodium nitrite (2.8 g, 40.5 mmol) in water (36 mL). After stirring for 2 hours on ice bath, the reaction mixture was extracted three times with dichloromethane. The organic extracts were dried over sodium sulfate, filtered and evaporated to give the product. ¹H NMR (300 MHz, CDCl₃) δ 8.20 (d,1H), 7.70 (d,1H), 4.05 (d, 3H), 3.55 (s, 3H) Positive electrospray LC-MS, m/e 195 (M+H⁺).

Step 2: Preparation of methyl 4-(1-methylhydrazino)benzoate.

To a suspension of the product of step 1 (3.0 g, 15.4 mmol) in glacial acetic acid (30 mL) at −10 deg C. was added activated zinc dust (3.0 g, 46.2 mmol) in water (15 mL). After stirring for 2 hours on ice bath, the suspension was filtered through celite. The celite cake was washed with methanol repeatedly. The organic extracts were dried over sodium sulfate, filtered, concentrated and purified by silica flash column chromatography to give final product (300 mg, 15% yield). ¹H NMR (400 MHz, CDCl₃) δ 7.90 (d,1H), 6.95 (d, 1H), 3.87 (d, 3H), 3.30 (s, 3H) Positive electrospray LC-MS, m/e 181 (M+H⁺).

### EXAMPLE 854

This example illustrates the preparation of 2-[5-Fluoro-2-(2-fluorophenyl)pyrimidin-4-yl]-1,5,6,7-tetrahydro-4H-pyrrolo[3,2-c]pyridin-4-one.

Step 1: 2,4-Dichloro-5-fluroropyrimidine was prepared by a literature method [Mike Butters et al, Organic Process Research & Development, 5(1), 28-36, 2001] from 5-fluorouracil. ¹H NMR (400 MHz, CDCl₃) δ 8.47 (d, 1H). Positive electrospray GC-MS, m/e (M+): 167.

Step 2: Preparation of 2-chloro-4-(1-ethoxyvinyl)-5-fluoropyrimidine.

The mixture of the product from step 1(815 mg, 4.9 mmol), tributyl(1-ethoxyvinyl)tin (1.65 ml, 4.9 mmol), and tetrakis(triphenylphosphine)palladium (200 mg, 0.17 mmol) in toluene (8 ml) was deoxygenated and heated to 80C overnight, then cooled to room temperature. The reaction mixture was purified by flash column chromatography to give 0.85 g colorless oil. ¹H NMR (400 MHz, CDCl₃) δ 8.37 (d, 1H), 5.19 (d, 1H), 4.60 (d, 1H), 3.86 (m, 2H), 1.31 (m, 3H). Positive electrospray GC-MS, m/e (M+): 202.

Step 3: Preparation of 1-(2-chloro-5-fluoropyrimidin-4-yl)ethanone.

Concentrated HCl in water (0.5 ml) was added dropwise into the solution of the product from step 2 (800 mg) in THF (8 ml). The mixture was stirred at room temperature for one hour, then was diluted with dichloromethane (10 ml), washed with water 3 times, and brine. The organic layer was dried over magnesium sulfate and concentrated to give 0.45 g light yellow oil. Positive electrospray GC-MS, m/e (M+): 174.

Step 4: Preparation of 2-bromo-1-(2-chloro-5-fluoropyrimidin-4-yl)ethanone.

The product of step 3 (440 mg, 2.5 mmol) was dissolved in glacial acetic acid (2.2 ml) and treated with bromine (0.13 ml, 2.5 mmol) followed by HBr/AcOH (30% w/v, 0.56 ml, 2.5 mmol). After 3 hours of stirring, the brown solution was diluted with dichloromethane (10 ml), washed with sodium carbonate solution, water, and brine. The organic layer was dried over magnesium sulfate and concentrated to give 0.64 g light yellow oil. Positive electrospray GC-MS, m/e (M+): 252.

Step 5: Preparation of 2-(2-chloro-5-fluoropyrimidin-4-yl)-1,5,6,7-tetrahydro-4H-pyrrolo[3,2-c]pyridin-4-one.

The product of step 4 (640 mg, 2.5 mmol) was combined in absolute ethanol (5 ml) with ammonium acetate (854 mg, 11 mmol) and 2,4-dixoxpiperdine (300 mg, 2.6 mmol). After 1 hour, the solvent was removed. The brown oily residue was washed with water, then dissolved in DMF/water, and purified by prep. HPLC to give 80 mg pure product as an off white solid. ¹HNMR (400 MHz, DMSO-d₆) δ 12.29 (s, 1H), 8.65 (d, 1H), 7.21 (s, 1H), 7.07 (d, 1H), 3.36 (m, 2H), 2.45 (m, 2H). Positive electrospray LC-MS, m/e (M+H): 267.

Step 6: Preparation of 2-[5-fluoro-2-(2-fluorophenyl)pyrimidin-4-yl]-1,5,6,7-tetrahydro-4H-pyrrolo[3,2-c]pyridin-4-one.

Using the standard Suzuki coupling conditions, the degassed mixture was heated to 80C overnight. Purified by prep. HPLC to give the title compound as a yellow solid [positive electrospray LC-MS, m/e (M+H): 327], plus by-products 2-[5-fluoro-2-(2-fluorophenyl)-1-oxidopyrimidin-4-yl]-1,5,6,7-tetrahydro-4H-pyrrolo[3,2-c]pyridin-4-one [positive electrospray LC-MS, m/e (M+H): 343], and 2-[2-(dimethylamino)-5-fluoropyrimidin-4-yl]-1,5,6,7-tetrahydro-4H-pyrrolo[3,2-c]pyridin-4-one [positive electrospray LC-MS, m/e (M+H): 276].

### EXAMPLE 855

This example illustrates the preparation of 2-(3-bromo-4-fluorophenyl)-1,5,6,7-tetrahydro-4H-pyrrolo[3,2-c]pyridin-4-one.

2.52 mL (0.0492 mole) bromine was added drop wise to a solution of 9.68g (0.0446 mole) 3′-bromo-4′-fluoroacetophenone (Aldrich) in 200 mL acetic acid at room temperature with stirring. 8.8 mL (0.0446 mole) HBr/acetic acid (30% w/v) (Aldrich) was then added in one portion and the reaction was stirred at room temperature for 4 hrs. The solvent was evaporated overnight under a stream of nitrogen at room temperature. The residue was slurried in hexane and the ppt. was filtered, washed with hexane and dried under vacuum to yield 3.5 g of product as an off-white solid. The solvent from the mother liquor was evaporated off and the residue was treated with hexane/ether. The resulting ppt. was filtered and dried to yield a second crop of 2.13 g as a white solid. Total yield of 2-bromo-1-(3-bromo-4-fluorophenyl) ethanone was 5.63 g.

3.2g (0.041 mole) ammonium acetate was then added to a solution of 3.05 g (0.0103 mole) 2-bromo-1-(3-bromo-4-fluorophenyl) ethanone in 32 mL of abs. ethanol at room temperature followed by 1.3 g (0.011 mole) of 2,4-dioxopiperidine (preparation previously described). The reaction was stirred at room temperature for 3 days, then at 50° C. for 2 days. The solvent was evaporated under a stream of nitrogen. Water was added to the residue and this was slurried overnight at room temperature. The resulting ppt. was filtered, washed with water and dried to yield 2.5 g of crude 2-(3-bromo-4-fluorophenyl)-1,5,6,7-tetrahydro-4H-pyrrolo[3,2-c]pyridin-4-one as an off-white solid. 200 mg. of this crude product was recrystallized from 60% ethanol/water to yield 50 mg of pure 2-(3-bromo-4-fluorophenyl)-1,5,6,7-tetrahydro-4H-pyrrolo[3,2-c]pyridin-4-one. ¹H NMR ((CD₃)₂SO, 400 MHz) δ 11.65 (s, 1H), 7.94 (dd, J=0.006, 0.01 Hz, 1H), 7.627-7.666 (m, 1H), 7.339, (t, J=0.022 Hz, 1H), 6.951 (s, 1H), 6.723 (d, J=0.006 Hz, 1H), 3.36 (dt, J=0.007, 0.01 Hz, 2H), 2.770 (t, J=0.017 Hz, 2H). HRMS [M+H]⁺ m/z Calculated for C₁₃H₁₀BrFN₂O: 309.0033. Found: 309.0019.

### EXAMPLE 856

This example illustrates the preparation of 2-(3′,6-difluoro-1,1′-biphenyl-3-yl)-1,5,6,7-tetrahydro-4H-pyrrolo[3,2-c]pyridin-4-one.

250 mg (0.0018 mole) 3-fluorophenylboronic acid (Aldrich) was added to 370 mg (0.0012 mole) 2-(3-bromo-4-fluorophenyl)-1,5,6,7-tetrahydro-4H-pyrrolo[3,2-c]pyridin-4-one in 6 mL anhyd. DMF, followed by 1.8 mL of 2M cesium carbonate. The reaction mixture was purged 3× with nitrogen. 100 mg (0.08 mmole) tetrakis (triphenylphosphine) palladium(0) was then added and the reaction was stirred overnight at 80° C. After cooling, the reaction mixture was acidified with TFA and the product isolated by rev. phase prep HPLC to yield (after lyophilization) 170 mg of 2-(3′,6-difluoro-1,1′-biphenyl-3-yl)-1,5,6,7-tetrahydro-4H-pyrrolo[3,2-c]pyridin-4-one as a white solid. ¹H NMR ((CD₃)₂SO, 400 MHz) δ 11.639 (s, 1H), 7.798 (dd, J=0.006, 0.012 Hz, 1H), 7.645-7.683 (m, 1H), 7.448-7.555 (m, 3H), 7.231-7.326 (m, 2H), 6.935 (s, 1H), 6.735 (d, J=0.006 Hz, 1H), 3.372 (t, J=0.017 Hz, 2H), 2.788 (t, J=0.017 Hz, 2H). HRMS [M+H]⁺ m/z Calculated for C₁₉H₁₄F₂N₂O: 325.1147. Found: 325.1165.

### EXAMPLE 857

This example illustrates the production of 5-methyl-2-(2-quinolin-3-ylpyridin-4-yl)-1,5,6,7-tetrahydro-4H-pyrrolo[3,2-c]pyridin-4-one trifluoroacetate.

Step 1. (Preparation of 2-(2-quinolin-3-ylpyridin-4-yl)-1,5,6,7-tetrahydro-4H-pyrrolo[3,2-c]pyridin-4-one).

A suspension of 2-(2-chloropyridin-4-yl)-1,5,6,7-tetrahydro-4H-pyrrolo[3,2-c]pyridin-4-one (4.3 g,17.3 mmol), 3-quinoline boronic acid (4.5 g, 26.0 mmol),. and tetrakis(triphenylphosphine) palladium (0) (1.0 g, 0.86 mmol) in 45 mL of dimethylformamide and 17.3 mL of 2.0 M cesium carbonate was heated to 83 degrees Celsius for 18 hours. The reaction was cooled to room temperature and poured into 400 mL of water. The resulting precipitate was filtered and dried to give the title compound as a grey solid (6.3 g, quantitative). m/z (M+H): 341.

Step 2. (Preparation of 2-(2-quinolin-3-ylpyridin-4-yl)-1-{[2-(trimethylsilyl)ethoxy]methyl}-1,5,6,7-tetrahydro-4H-pyrrolo[3,2-c]pyridin-4-one).

A suspension of 2-(2-quinolin-3-ylpyridin-4-yl)-1,5,6,7-tetrahydro-4H-pyrrolo[3,2-c]pyridin-4-one (1.15 g, 3.4 mmol) in 20 mL of dimethylformamide was cooled to zero degrees celcius and treated with a solution of lithium tert-butoxide 1.0 M in tetrahydrofuran (3.7 mL, 3.7 mmol) and stirred for 30 minuets. Trimethylsilylethoxymethyl chloride (0.65 mL, 3.7 mmol) was added to and stirred for 30 minuets at zero degrees celcius, allowed to warm to room temperature and poured into brine, extracted 3× with ethyl acetate, dried over magnesium sulfate, filtered and condensed to a solid. The solid was filtered and washed with hexanes to give the title compound as an off white solid (1.15 g, 2.4 mmol, 70%). m/z (M+H): 471

Step 3. (Preparation of 5-methyl-2-(2-quinolin-3-ylpyridin-4-yl)-1-{[2-(trimethylsilyl)ethoxy]methyl}-1,5,6,7-tetrahydro-4H-pyrrolo[3,2-c]pyridin-4-one).

A solution of 2-(2-quinolin-3-ylpyridin-4-yl)-1-{[2-(trimethylsilyl)ethoxy]methyl)-1,5,6,7-tetrahydro-4H-pyrrolo[3,2-c]pyridin-4-one (600 mg, 1.3 mmol) in 10.0 mL of dimethylformamide was cooled to zero degrees celcius and treated with a solution of lithium tert-butoxide 1.0 M in tetrahydrofuran (1.9 mL, 1.9 mmol) and stirred for 20 minuets. Methyl iodide (0.12 mL, 1.9 mmol) was added and the reaction allowed to warm to room temperature and poured into 150 mL of water and extracted with ethyl acetate, washed with brine, dried over magnesium sulfate, filtered and condensed. Purification by flash chromatography (gradient: 100% ethyl acetate to 15% methanol/ethyl acetate) gave the title compound as an off white solid (500 mg, 1.0 mmol, 80%). m/z (M+H): 484.

Step 4. (Preparation of 5-methyl-2-(2-quinolin-3-ylpyridin-4-yl)-1,5,6,7-tetrahydro-4H-pyrrolo[3,2-c]pyridin-4-one trifluoroacetate).

A solution of 5-methyl-2-(2-quinolin-3-ylpyridin-4-yl)-1-{[2-(trimethylsilyl)ethoxy]methyl}-1,5,6,7-tetrahydro-4H-pyrrolo[3,2-c]pyridin-4-one (250 mg, 0.5 mmol) in 4.0. mL ethyl alcohol was treated with 3.0 mL of a 3 M solution of hydrochloric acid and heated to 90 degrees celcius for three hours cooled to room temperature, added trifluoroacetic acid, filtered through a syringe filter (0.45 μm), purified by rpHPLC, and lyophilized to give the title compound as a yellow solid (150 mg, 0.4 mmol, 83%). ¹H NMR (400 MHz, DMSO-d₆) δ 12.18 (s, 1H), 9.68 (d, J=2.2 Hz, 1H), 9.20 (s, 1H), 8.70 (d, J=5.6 Hz, 1H), 8.55 (s, 1H), 8.16 (t, J=7.7 Hz, 1H), 7.90 (t, J=6.8 Hz, 1H), 7.85-7.70 (m, 2H), 7.44 (s, 1H), 3.59 (t, J=7.0 Hz, 2H), 3.01 (t, J=7.1 Hz, 2H), 2.94 (s, 3H). HRMS calculated for C₂₂H₁₈N₄O (MH⁺) 355.1553, found 355.1548. Anal. calculated for C₂₂H₁₈N₄O.1.5 TFA.1.2 H₂O C, 54.88; H, 4.03; N, 10.24. Found: C, 54.80; H, 4.00; N, 10.39.

2-(ArN(R)N═CR′)-Pyridine Analogs

Step 1: Preparation of 4-(4-oxo-4,5,6,7-tetrahydro-1H-pyrrolo[3,2-c]pyridin-2-yl)pyridine-2-carbaldehyde.

A suspension of 2-[2-(hydroxymethyl)pyridin-4-yl]-1,5,6,7-tetrahydro-4H-pyrrolo[3,2-c]pyridin-4-one trifluoroacetate (130 mg, 0.53 mmol) in methanol (3 mL) was stirred with activated manganese dioxide (144 mg, 1.62 mmol) under Ar at RT overnight. The suspension was filtered through celite. The celite cake was washed with methanol repeatedly. The filtrated was concentrated to give a white solid (110 g, 90% yield, >90% pure), which was used for next step without further purification. ¹H NMR (300 MHz, CD₃OD) δ 10.1 (s, 1H), 8.52 (s, 1H), 8.50 (d, J=6.8 Hz, 1H), 7.95 (d, J=7.2 Hz, 1H), 7.50 (s, 1H), 3.59 (t, J=6.9 Hz, 2H), 3.02 (t, J=6.9 Hz, 2H). Positive electrospray LC-MS, m/e 242 (M+H⁺).

Step 2: Synthesis of hydrazones and oximes.

A solution of of the product from step 1 or the compound synthesized according to Example 782 (100 mg, 0.40 mmol) in dimethylformamide (3.0 mL) was treated with an aryl hydrazine or O-arylhydroxylamine (0.53 mmol) selected from the compound synthesized according to any of Example 856, Example 857, and commercially available compounds. The reaction mixtures were stirred at room temperature for 1 hour under Ar, then filtered through a syringe filter (0.45 μm), purified by prep. rpHPLC, and lyophilized to give the products, which were characterized by analytical reverse phase HPLC, NMR, and MS. The following compounds were prepared by this method.

| | | Calculated | |
|---|---|---|---|
| Example | | Exact Mass | Found |
| No. | Compound | M + H | M + H |
| EXAMPLE 858 | 4-(4-oxo-4,5,6,7-tetrahydro- | 332.1506 | 332.1504 |
| | 1H-pyrrolo[3,2-c]pyridin-2- | | |
| | yl)pyridine-2-carbaldehyde | | |
| | phenylhydrazone | | |
| EXAMPLE 859 | 4-(4-oxo-4,5,6,7-tetrahydro- | 346.1662 | 346.1636 |
| | 1H-pyrrolo[3,2-c]pyridin-2- | | |
| | yl)pyridine-2-carbaldehyde | | |
| | methyl(phenyl)hydrazone | | |
| EXAMPLE 860 | 4-(4-oxo-4,5,6,7-tetrahydro- | 333.1346 | 333.1374 |
| | 1H-pyrrolo[3,2-c]pyridin-2- | | |
| | yl)pyridine-2-carbaldehyde | | |
| | O-phenyloxime | | |
| EXAMPLE 861 | 2-{2-[(1E)-N- | 346.1662 | 346.1681 |
| | phenylethanehydrazonoyl]pyridin- | | |
| | 4-yl}-1,5,6,7-tetrahydro-4H- | | |
| | pyrrolo[3,2-c]pyridin-4-one | | |
| EXAMPLE 862 | 4-(4-oxo-4,5,6,7-tetrahydro- | 417.2034 | 417.2034 |
| | 1H-pyrrolo[3,2-c]pyridin-2- | | |
| | yl)pyridine-2-carbaldehyde | | |
| | (2-morpholin-4-ylphenyl)hydrazone | | |
| EXAMPLE 863 | 4-(4-oxo-4,5,6,7-tetrahydro- | 350.1412 | 350.1437 |
| | 1H-pyrrolo[3,2-c]pyridin-2- | | |
| | yl)pyridine-2-carbaldehyde | | |
| | (3-fluorophenyl)hydrazone | | |
| EXAMPLE 864 | 4-((2E)-2-{[4-(4-oxo-4,5,6,7- | 376.1404 | 376.1427 |
| | tetrahydro-1H-pyrrolo[3,2- | | |
| | c]pyridin-2-yl)pyridin-2- | | |
| | yl]methylene}hydrazino)benzoic | | |
| | acid | | |
| EXAMPLE 865 | 4-(4-oxo-4,5,6,7-tetrahydro- | 512.2769 | 512.2747 |
| | 1H-pyrrolo[3,2-c]pyridin-2- | | |
| | yl)pyridine-2-carbaldehyde | | |
| | (4-{[(2R)-2-(pyrrolidin-1- | | |
| | ylmethyl)pyrrolidin-1- | | |
| | yl]carbonyl}phenyl)hydrazone | | |
| | trifluoroacetate | | |
| EXAMPLE 866 | 4-(4-oxo-4,5,6,7-tetrahydro- | 445.191 | 445 (ES) |
| | 1H-pyrrolo[3,2-c]pyridin-2- | | |
| | yl)pyridine-2-carbaldehyde | | |
| | [4-(morpholin-4- | | |
| | ylcarbonyl)phenyl]hydrazone | | |
| EXAMPLE 867 | 4-(4-oxo-4,5,6,7-tetrahydro- | 429.1961 | 429.1997 |
| | 1H-pyrrolo[3,2-c]pyridin-2- | | |
| | yl)pyridine-2-carbaldehyde | | |
| | [4-(pyrrolidin-1- | | |
| | ylcarbonyl)phenyl]hydrazone | | |
| EXAMPLE 868 | 4-(4-oxo-4,5,6,7-tetrahydro- | 410.1281 | 410.1277 |
| | 1H-pyrrolo[3,2-c]pyridin-2- | | |
| | yl)pyridine-2-carbaldehyde [4- | | |
| | (methylsulfonyl)phenyl]hydrazone | | |
| EXAMPLE 869 | methyl 4-((2E)-1 -methyl-2-{[4- | 404.1717 | 404.1757 |
| | (4-oxo-4,5,6,7-tetrahydro-1H- | | |
| | pyrrolo[3,2-c]pyridin-2- | | |
| | yl)pyridin-2- | | |
| | yl]methylene}hydrazino)benzoate | | |
| EXAMPLE 870 | 4-(4-oxo-4,5,6,7-tetrahydro- | 436.1364 | 436.1386 |
| | 1H-pyrrolo[3,2-c]pyridin-2- | | |
| | yl)pyridine-2-carbaldehyde (2,4- | | |
| | dinitrophenyl)(methyl)hydrazone | | |
| EXAMPLE 871 | 4-((2E)-1-methyl-2-{[4-(4-oxo- | 390.1561 | 390.1583 |
| | 4,5,6,7-tetrahydro-1H- | | |
| | pyrrolo[3,2-c]pyridin-2- | | |
| | yl)pyridin-2- | | |
| | yl]methylene}hydrazino)benzoic | | |
| | acid trifluoroacetate | | |
| EXAMPLE 872 | 4-(4-oxo-4,5,6,7-tetrahydro- | 459.1387 | 459.1365 |
| | 1H-pyrrolo[3,2-c]pyridin-2- | | |
| | yl)pyridine-2-carbaldehyde | | |
| | methyl[2-nitro-4- | | |
| | (trifluoromethyl)phenyl]hydrazone | | |
| | trifluoroacetate | | |
| EXAMPLE 873 | 4-(4-oxo-4,5,6,7-tetrahydro- | 362.1612 | 362.1602 |
| | 1H-pyrrolo[3,2-c]pyridin-2- | | |
| | yl)pyridine-2-carbaldehyde (4- | | |
| | methoxyphenyl)hydrazone | | |
| | trifluoroacetate | | |
| EXAMPLE 874 | 4-(4-oxo-4,5,6,7-tetrahydro- | 459.2139 | 459.2153 |
| | 1H-pyrrolo[3,2-c]pyridin-2- | | |
| | yl)pyridine-2-carbaldehyde | | |
| | methyl[4-(morpholin-4- | | |
| | ylcarbonyl)phenyl]hydrazone | | |
| | trifluoroacetate | | |

### EXAMPLE 875

This example illustrates the preparation of N-{4-[4-(4-oxo-4,5,6,7-tetrahydro-1H-pyrrolo[3,2-c]pyridin-2-yl)pyridin-2-yl]phenyl}-2-pyridin-4-ylacetamide bis(trifluoroacetate).

To the solution of pyridin-4-ylacetic acid hydrochloride (140 mg, 0.81 mMol) in DMF (2.0 mL) at room temperature under nitrogen was added carbonyidiimidazole (158 mg, 0.972 mMol). 30 minutes later, 2-[2-(4-aminophenyl)pyridin-4-yl]-1,5,6,7-tetrahydro-4H-pyrrolo[3,2-c]pyridin-4-one (200 mg, 0.478 mMol) was added into the mixture followed by N-methylmorpholine (58 mg, 0.57 mMol). The resulting mixture was stirred at room temperature for overnight. After acidification to pH=1.0 by TFA, the mixture was purified by reversed phase prep HPLC. The resulting solid was further purified by flash chromatography and eluted with a gradient of 10% MeOH/EtOAc (100 mL) to 20% MeOH/EtOAc (100 mL) and 100% MeOH. Desired fractions were combined and concentrated and redissolved in a mixture of acetonitrile/water and freeze-dried to give a yellowish solid. ¹H NMR (400 MHz, CD3OD) δ (ppm): 8.79 (d, J=6.8 Hz, 2H), 8.49 (d, J=6.4 Hz, 1H), 8.33 (d, J=2.0 Hz, 1H), 8.06 (d, J=6.8 Hz, 2H), 7.90-7.96 (m, 5H), 7.53 (s, 1H), 4.16 (d, J=7.2 Hz, 1H), 3.58-3.63 (m, 3H), 3.02 (t, J=6.8 Hz, 2H). Theoretical high resolution Mass (M+H) for C₂₅H₂₂N₅O₂: 424.1768; Found: 424.1779.

### EXAMPLE 876.

This example illustrates that MK2 knock-out mice (MK2 (−/−)) are resistant to the formation of K/BN serum-induced arthritis and that compounds that inhibit MK-2 should be effective for the prevention and treatment of TNFα-mediated diseases or disorders.

A strain of mice has been reported that develops symptoms similar to human rheumatoid arthritis. The mice were designated K/BxN mice. See, Wipke, B. T. and P. M. Allen, J. of Immunology, 167:1601-1608 (2001). Serum from the mice can be injected into host animals to provoke a typical RA response. The progression of the RA symptoms in the mice is measured by measuring paw thickness as a function of time.

In the present example, host mice having normal MK-2 production (MK2 (+/+)) were genetically altered by disabling the gene encoding MK-2 to produce mice having no capability of endogenous synthesis of active MK-2 (MK2 (−/−)). Normal host mice (MK2 (+/+)) and MK-2 knock-out mice (MK2 (−/−), were separated into four groups with each group containing both male and female mice. All groups of mice were treated similarly, except that one group (Normal), composed of MK2 (+/+) mice that served as the control group, was not injected with serum from K/BxN mice, while the other three groups were injected with K/BxN serum at day 0. The other three groups of mice were MK2 (+/+), MK2 (−/−), and Anti-TNF. The Anti-TNF group was composed of MK2 (+/+) mice which were also injected at day) with anti-TNF antibody. The paw thickness of all mice was measured immediately after the injections on day 0, and then on each successive day thereafter for 7 days.

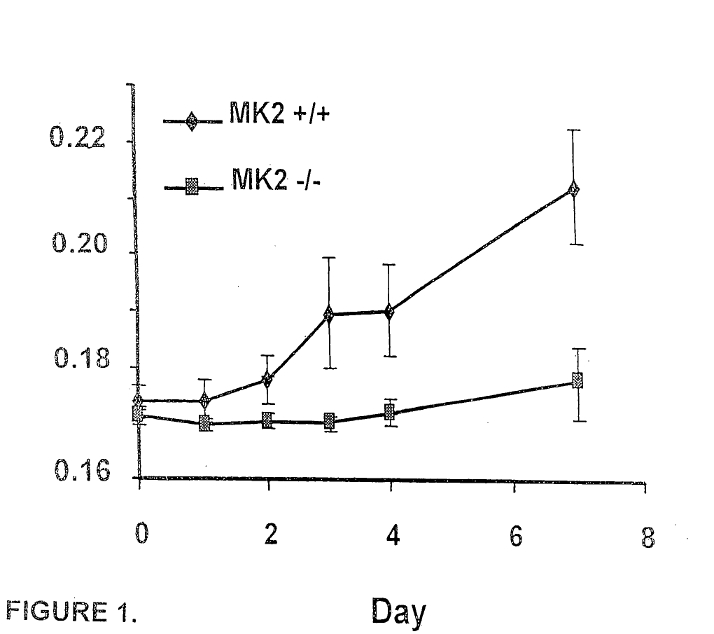

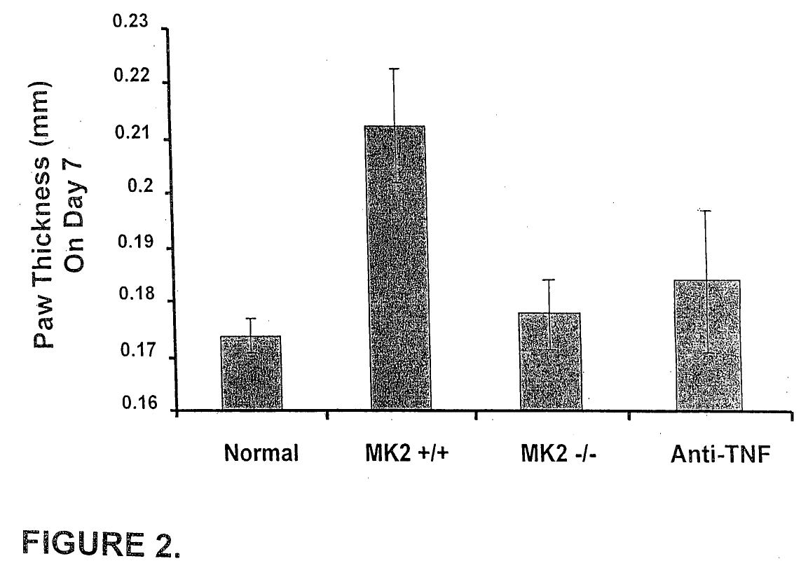

FIG. 1 is a graph that shows paw thickness as a function of time from day 0 to day 7 for MK2 (+/+) and MK2 (−/−) mice, which have received serum injection. It can be seen that paw thickness increased significantly for MK2(+/+) mice, whereas there was substantially no increase in paw thickness for MK2 knock-out mice. This indicated the requirement for a functioning MK2 regulatory system to the inflammatory response caused by the serum challenge. When anti-TNF antibody was administered to the MK2 (+/+) mice along with the serum injection, the swelling response was significantly reduced. This can be seen in FIG. 2, which is a bar chart showing paw thickness at seven days after injection for normal mice, MK2 (+/+) mice receiving serum, MK2 (−/−) mice receiving serum, and MK2 (+/+) mice receiving serum and anti-TNF antibody.

This data shows that the MK2 knock-out mice show no arthritic response to a serum challenge, whereas MK2 (+/+) mice show a normal response. Treatment of MK2 (+/+) mice that receive a serum challenge with anti-TNF antibody reduces the response back to near-normal levels. This illustrates the utility of the MK2 regulatory system as a potential control point for the modulation of TNF production, and indicates that such regulation could serve as a treatment for inflammation—such as that caused by arthritis, for example. It further shows that MK2 inhibition can have a beneficial effect on inflammation, and indicates that administration of an MK2 inhibitor can be an effective method of preventing or treating TNF modulated diseases or disorders.

### EXAMPLE 877.

This illustrates the efficacy of 2-{2-[(E)-2-phenylethenyl]pyridin-4-yl}-1,5,6,7-tetrahydro-4H-pyrrolo[3,2-c]pyridin-4-one and 2-[2-(2-fluorophenyl)pyridin-4-yl]-1,5,6,7-tetrahydro-4H-pyrrolo[3,2-c]pyridin-4-one trifluoroacetate for the prevention and treatment of SCW arthritis in rats, and also shows that those compounds exhibit a typical dose-response relationship.

The use of Lewis rats with chronic streptococcal cell wall (SCW)-induced arthritis as models for testing potentially therapeutic compounds has been described by Richards, P. J., et al., Rhermatology (Oxford), 40(9):978-987 (2001), and Melay, L. M. et al, Bioorg. Med. Chem., 9(2):537-554 (2001), among others. In the present test, female Lewis rats were divided into eight groups. One group served as the “normal” control and received no arthritis inducement. Chronic streptococcal cell wall (SCW)-induced arthritis was induced in the remaining seven groups. One of the severn groups in which arthritis was induced served as a “vehicle” control and received dosage only of vehicle at the same intervals as those rats receiving the test compounds. Three additional groups of rats received the vehicle plus 2-{2-[(E)-2-phenylethenyl]pyridin-4-yl}-1,5,6,7-tetrahydro-4H-pyrrolo[3,2-c]pyridin-4-one (Compound “A”) in daily dosages. All rats were started on the test at day 0. At day ten (10) administration of the test compounds was started with one group receiving Compound A at a dose level of 200 mpk/day (milligrams/kilogram/day), another group receiving the compound at 60 mpk/day, and the third group receiving the test compound at 20 mpk/day. Three additional groups were treated similarly, but received daily dosages of 2-[2-(2-fluorophenyl)pyridin-4-yl]-1,5,6,7-tetrahydro-4H-pyrrolo[3,2-c]pyridin-4-one trifluoroacetate (Compound “B”) at levels of 240 mpk/day, 120 mpk/day, and 60 mpk/day, respectively.

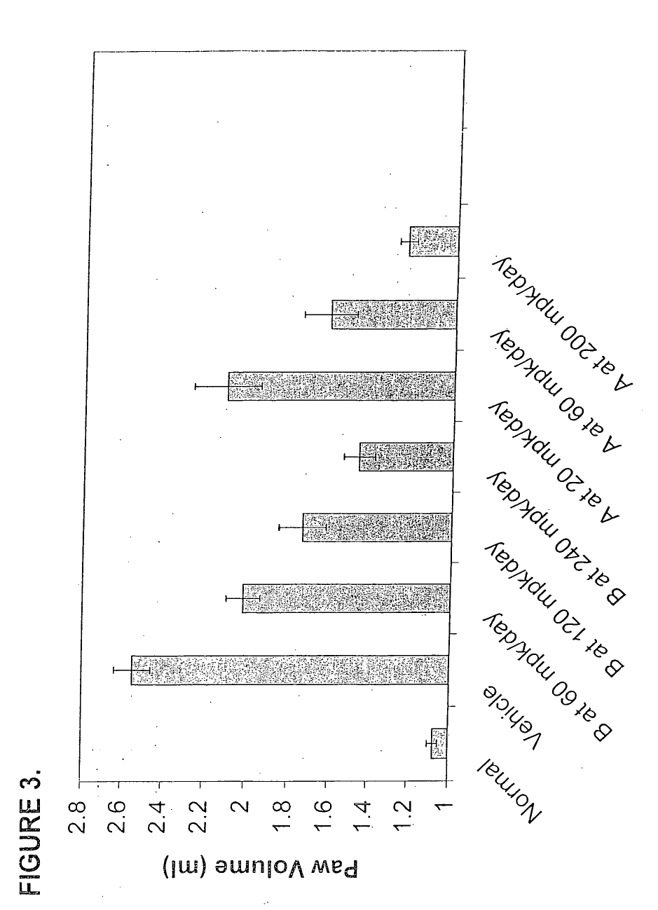

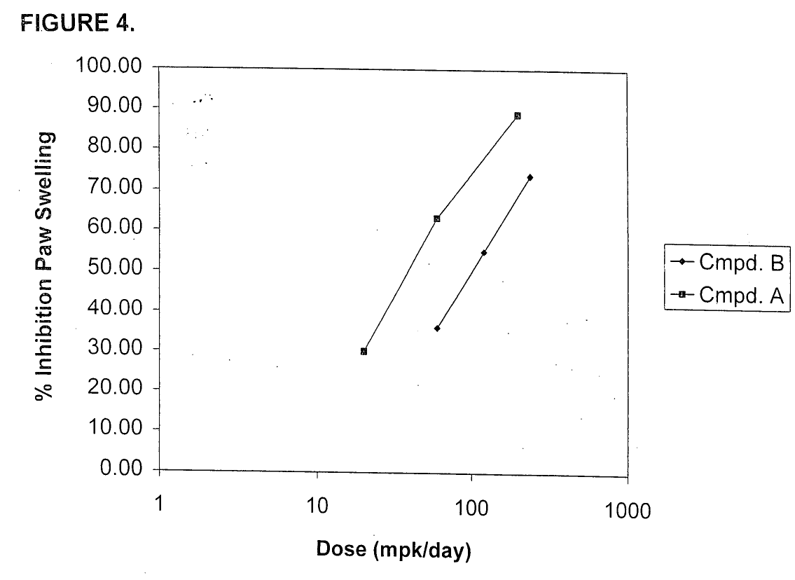

To measure the effectiveness of the compounds for the treatment of SCW-induced arthritis, rat paw volume was measured at eleven (11) days from the initiation of administration of the test compounds. The data are shown in FIG. 3, which is a plot of average paw volume as a function of the treatment regimin. The data show a negligible increase in paw volume for the normal group and an expected significant increase for the SCW-induced group receiving only vehicle. Rats receiving both test compounds A and B showed significantly lower increases in paw volume than the “vehicle” controls, with both compounds showing increased efficacy with an increase in dosage. FIG. 4 shows a semi-log plot of percent inhibition in paw swelling as a function of the dosage rate for each of the two test compounds. The data indicate a typical dose-response relationship for each of the two compounds. Both compounds are shown to be effective for the treatment of arthritis in SCW-induced rates.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. An MK-2 inhibiting compound having the structure: 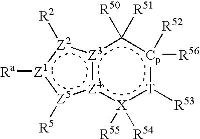
where:
Z¹, Z³ and Z⁴ are independently selected from carbon, and nitrogen;
Z² and Z⁵ are independently selected from carbon, nitrogen, sulfur, and oxygen, and join together with Z¹, Z³ and Z⁴ to form a ring that is selected from a pyrrole, furan, thiophene, oxazole, thiazole, triazole, and imidazole;
when either Z², or Z⁵ is oxygen or sulfur, it has no substituent group;
when Z¹, Z², Z³, Z⁴, and Z⁵ form an imidazole ring, Z¹ is carbon and if Z² and Z⁵ are nitrogen, one is unsubstituted and Z³ and Z⁴ are carbon, if Z³ and Z⁵ are nitrogen, Z⁵ is unsubstituted and Z² and Z⁴ are carbon, and if Z² and Z⁴ are nitrogen, Z² is unsubstituted and Z³ and Z⁵ are carbon;
when Z¹, Z², Z³, Z⁴, and Z⁵ form an oxazole or thiazole ring, Z¹, Z³ and Z⁴ are carbon and one of Z² and Z⁵ is nitrogen that is unsubstituted;
when Z¹, Z², Z³, Z⁴, and Z⁵ form a triazole ring, Z² and Z⁵ are nitrogen that is unsubstituted;
T is selected from C and N;
p is an integer selected from 0,1,2 and 3;
X is selected from C and S;
R^{a} is selected from: 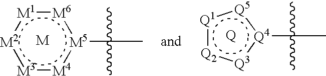
where dashed lines indicate optional single or double bonds;
when ring M is aromatic, M⁵ is carbon and each of M¹, M², M³, M⁴ and M⁶ is independently selected from CR^{b} and N;
when ring M is partially saturated, M⁵ is carbon and each of M¹, M², M³ M⁴ and M⁶ is independently selected from CR^{b}, N, C(R^{b})₂, NR^{b}, oxygen and sulfur;
when ring Q is heteroaromatic, at least one of Q¹, Q², Q³, Q⁴, and Q⁵ is other than carbon, Q⁴ is optionally C or N, and Q¹, Q², Q³, and Q⁵ are each independently selected from CR^{b}, NR^{b} and N; optionally, Q⁴ is C, Q¹ is CR^{b}, and one of Q², Q³, and Q⁵ is optionally oxygen, NR^{b}, or sulfur, and the remainder of Q², Q³, and Q⁵ are independently selected from CR^{b} and N;
when ring Q is partially saturated, Q¹ is optionally CR^{b}, NR^{b}, or N, and Q⁴ is optionally C or N; one of Q², Q³ and Q⁵ is optionally oxygen or sulfur, and the remainder of Q², Q³ and Q⁵ are independently selected from CR^{b}, N, C(R^{b})₂, and NR^{b};
R^{b} is selected from —H, C₁-C₆ alkyl, C₂-C₆ alkenyl, C₂-C₆ alkynyl, C₁-C₆ alkyl-R¹¹, C₂-C₆ alkenyl-R¹¹, C₂-C₆ alkynyl-R¹¹, C₁-C₆ alkyl-(R¹¹)₂, C₂-C₆ alkenyl-(R¹¹)₂, CSR¹¹, amino, NHR⁷, NR⁸R⁹, N(R⁷)—N(R⁸)(R⁹), C(R¹¹)═N—N(R⁸)(R⁹), N═N(R⁷), N(R⁷)—N═C(R⁸), C(R¹¹)═N—O(R¹⁰), ON═C(R¹¹), C₁-C₆ alkyl-NHR⁷, C₁-C₆ alkyl-NR⁸R⁹, (C₁-C₄)alkyl-N(R⁷)—N(R⁸)(R⁹), (C₁-C₄)alkylC(R¹¹)═N—N(R⁸)(R⁹), (C₁-C₄)alkyl-N═N(R⁷), (C₁-C₄)alkyl-N(R⁷)—N═C(R⁸), nitro, cyano, O—R¹⁰, C₁-C₄ alkyl-OR¹⁰, COR¹¹, SR¹⁰, SSR¹⁰, SOR¹¹, SO₂R¹¹, C₁-C₆ alkyl-COR¹¹, C₁-C₆ alkyl-SR¹⁰, C₁-C₆ alkyl-SOR¹¹, C₁-C₆ alkyl-SO₂R¹¹, halo, Si(R¹¹)₃, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹²;
R⁷, R⁸ and R⁹ are each independently selected from —H, C₁-C₆ alkyl, C₂-C₆ alkenyl, C₂-C₆ alkynyl, C₁-C₄ alkyl-R¹¹, C₁-C₆ alkyl-NHR¹³, C₁-C₆ alkyl-NR¹³R¹⁴, O—R¹⁵, C₁-C₄ alkyl-OR¹⁵, CO₂R¹⁵, C(S)OR¹⁵, C(O)SR¹⁵, C(O)R¹⁷, C(S)R¹⁷, CONHR¹⁶, C(S)NHR¹⁶, CON(R¹⁶)₂, C(S)N(R¹⁶)₂, SR¹⁵, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;
R¹⁰ is selected from —H, C₁-C₆ alkyl, C₂-C₆ alkenyl, C₂-C₆ alkynyl, C₁-C₆ alkyl-NHR¹³, C₁-C₆ alkyl-NR¹³R¹⁴, C₁-C₄ alkyl-OR¹⁵, CSR¹¹, CO₂R¹⁵, C(S)OR¹⁵, C(O)SR¹⁵, COR¹⁷, C(S)R¹⁷, CONHR¹⁶, C(S)NHR¹⁶, CON(R¹⁶)₂, C(S)N(R¹⁶)₂, SOR¹⁷, SO₂R¹⁷, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;
R¹¹ is selected from —H, C₁-C₆ alkyl, C₁-C₆ alkoxy, C₂-C₆ alkenyl, C₂-C₆ alkynyl, amino, NHR¹³, NR¹³R¹⁴, N═NR¹³, C₁-C₆ alkyl-NHR¹³, C₁-C₆ alkyl-NR¹³R¹⁴, O—R¹⁵, C₁-C₄ alkyl-OR¹⁵, SR¹⁵, C₁-C₆ alkyl-CO₂R¹⁵, C₁-C₆ alkyl-C(S)OR¹⁵, C₁-C₆ alkyl-C(O)SR¹⁵, C₁-C₆ alkyl-COR¹⁷, C₁-C₆ alkyl-C(S)R¹⁷, C₁-C₆ alkyl-CONHR¹⁶, C₁-C₆ alkyl-C(S)NHR¹⁶, C₁-C₆ alkyl-CON(R¹⁶)₂, C₁-C₆ alkyl-C(S)N(R¹⁶)₂, C₁-C₆ alkyl-SR¹⁵, C₁-C₆ alkyl-SOR¹⁷, C₁-C₆ alkyl-SO₂R¹⁷, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;
R¹² is selected from —H, OH, C₁-C₁₀ alkyl, C₂-C₁₀ alkenyl, C₂-C₁₀ alkynyl, C₁-C₁₀ alkyl-R¹¹, C₂-C₁₀ alkenyl-R¹¹, C₂-C₁₀ alkynyl-R¹¹, C₁-C₁₀ alkyl-(R¹¹)₂, C₂-C₁₀ alkenyl-(R¹¹)₂, CSR¹¹, amino, NHR⁷, NR⁸R⁹, N(R⁷)—N(R⁸)(R⁹), C(R¹¹)═N-N(R⁸)(R⁹), N═N(R⁷), N(R⁷)-N═C(R⁸), C(R¹¹)═N—O(R¹⁰), ON═C(R¹¹), C₁-C₁₀ alkyl-NHR⁷, C₁-C₁₀ alkyl-NR⁸R⁹, (C₁-C₁₀)alkyl-N(R⁷)-N(R⁸)(R⁹), (C₁-C₁₀)alkylC(R¹¹)═N—N(R⁸)(R⁹), (C₁-C₁₀)alkyl-N═N(R⁷), (C₁-C₁₀)alkyl-N(R⁷)-N═C(R⁸), SCN, NCS, C₁-C₁₀ alkyl SCN, C₁-C₁₀ alkyl NCS, nitro, cyano, O—R¹⁰, C₁-C₁₀ alkyl-OR¹⁰, COR¹¹, SR¹⁰, SSR¹⁰, SOR¹¹, SO₂R¹¹, C₁-C₁₀ alkyl-COR¹¹, C₁-C₁₀ alkyl-SR¹⁰, C₁-C₁₀ alkyl-SOR¹¹, C₁-C₁₀ alkyl-SO₂R¹¹, halo, Si(R¹¹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R¹⁸;
R¹³ and R¹⁴ are each independently selected from —H, C₁-C₆ alkyl, C₂-C₆ alkenyl, C₂-C₆ alkynyl, C₁-C₄ alkyl-R²³, C₁-C₆ alkyl-NHR¹⁹, C₁-C₆ alkyl-NR¹⁹R²⁰, O—R²¹, C₁-C₄ alkyl-OR²¹, CO₂R²¹, C(S)OR²¹, C(O)SR²¹, C(O)R²³, C(S)R²³, CONHR²², C(S)NHR²², CON(R²²)₂, C(S)N(R²²)₂, SR²¹, SOR²³, SO₂R²³, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C1-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁵ and R¹⁶ are independently selected from —H, C₁-C₆ alkyl, C₂-C₆ alkenyl, C₂-C₆ alkynyl, C₁-C₆ alkyl-NHR¹⁹, C₁-C₆ alkyl-NR¹⁹R²⁰, C₁-C₄ alkyl-OR²¹, CSR¹¹, CO₂R²², COR²³, CONHR²², CON(R²²)₂, SOR²³, SO₂R²³, C₁-C₆ alkyl-CO₂R²², C₁-C₆ alkyl-COR²³, C₁-C₆ alkyl-CONHR²², C₁-C₆ alkyl-CON(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁷ is selected from —H, C₁-C₆ alkyl, C₂-C₆ alkenyl, C₂-C₆ alkenyl-R¹⁹, C₁-C₆ alkyl-R¹⁹, C2-C₆ alkynyl, amino, NHR¹⁹, NR¹⁹R²⁰, C₁-C₆ alkyl-NHR¹⁹, C₁-C₆ alkyl-NR¹⁹R²⁰, O—R²¹, C₁-C₄ alkyl-OR²¹, SR²¹, C₁-C₆ alkyl-CO₂R²¹, C₁-C₆ alkyl-C(S)OR²¹, C₁-C₆ alkyl-C(O)SR²¹, C₁-C₆ alkyl-COR²³, C₁-C₆ alkyl-C(S)R²³, C₁-C₆ alkyl-CONHR²², C₁-C₆ alkyl-C(S)NHR²², C₁-C₆ alkyl-CON(R²²)₂, C₁-C₆ alkyl-C(S)N(R²²)₂, C₁-C₆ alkyl-SR²¹, C₁-C₆ alkyl-SOR²³, C₁-C₆ alkyl-SO₂R²³, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁸ is selected from —H, OH, C₁-C₁₀ alkyl, C₂-C₁₀ alkenyl, C₂-C₁₀ alkynyl, C₁-C₁₀ alkyl-R²³, C₂-C₁₀ alkenyl-R²³, C₂-C₁₀ alkynyl-R²³, C₁-C₁₀ alkyl-(R²³)₂, C₂-C₁₀ alkenyl-(R²³)₂, CSR²³, amino, NHR¹⁹, NR²⁰R²⁰, N(R¹⁹)—N(R²⁰)(R²⁰), C(R²³)═N—N(R²⁰)(R²⁰), N═N(R¹⁹), N(R¹⁹)—N═C(R²⁰), C(R²³)═N—O(R²¹), ON═C(R²³), C₁-C₁₀ alkyl-NHR¹⁹, C₁-C₁₀ alkyl-NR²⁰R²⁰, (C₁-C₁₀)alkyl-N(R¹⁹)—N(R²⁰) (R²⁰), (C₁-C₁₀)alkylC(R²³)═N—N(R²⁰)(R²⁰), (C₁-C₁₀)alkyl-N═N(R¹⁹), (C₁-C₁₀)alkyl-N(R¹⁹)—N═C(R²⁰), SCN, NCS, C₁-C₁₀ alkyl SCN, C₁-C₁₀ alkyl NCS, nitro, cyano, O—R²¹, C₁-C₁₀ alkyl-OR²¹, COR²³, SR²¹, SSR²¹, SOR²³, SO₂R²³, C₁-C₁₀ alkyl-COR²³, C₁-C₁₀ alkyl-SR²¹, C₁-C₁₀ alkyl-SOR²³, C₁-C₁₀ alkyl-SO₂R²³, halo, Si(R²³)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R²⁴;
R¹⁹ and R²⁰ are each independently selected from —H, C₁-C₆ alkyl, C₂-C₆ alkenyl, C₂-C₆ alkynyl, C₁-C₄ alkyl-R²⁹, C₁-C₆ alkyl-NHR²⁵, C₁-C₆ alkyl-NR²⁵R²⁶, O—R²⁷, C₁-C₄ alkyl-OR²⁷, CO₂R²⁷, C(S)OR²⁷, C(O)SR²⁷, C(O)R²⁹, C(S)R²⁹, CONHR²⁸, C(S)NHR²⁸, CON(R²⁸)₂, C(S)N(R²⁸)₂, SR²⁷, SOR²⁹, SO₂R²⁹, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²¹ and R²² are independently selected from —H, C₁-C₆ alkyl, C₂-C₆ alkenyl, C₂-C₆ alkynyl, C₁-C₆ alkyl-NHR²⁵, C₁-C₆ alkyl-NR²⁵R²⁶, C₁-C₄ alkyl-OR²⁷, CSR¹¹, CO₂R²⁸, COR²⁹, CONHR²⁸, CON(R²⁸)₂, SOR²⁹, SO₂R²⁹, C₁-C₆ alkyl-CO₂R²⁸, C₁-C₆ alkyl-COR²⁹, C₁-C₆ alkyl-CONHR²⁸, C₁-C₆ alkyl-CON(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²³ is selected from —H, C₁-C₆ alkyl, C₂-C₆ alkenyl, C₂-C₆ alkenyl-R²⁵, C₁-C₆ alkyl-R²⁵, C2-C₆ alkynyl, amino, NHR²⁵, NR²⁵R²⁶, C₁-C₆ alkyl-NHR²⁵, C₁-C₆ alkyl-NR²⁵R²⁶, O—R²⁷, C₁-C₄ alkyl-OR²⁷, SR²⁷, C₁-C₆ alkyl-CO₂R²⁷, C₁-C₆ alkyl-C(S)OR²⁷, C₁-C₆ alkyl-C(O)SR²⁷, C₁-C₆ alkyl-COR²⁹, C₁-C₆ alkyl-C(S)R²⁹, C₁-C₆ alkyl-CONHR²⁸, C₁-C₆ alkyl-C(S)NHR²⁸, C₁-C₆ alkyl-CON(R²⁸)₂, C₁-C₆ alkyl-C(S)N(R²⁸)₂, C₁-C₆ alkyl-SR²⁷, C₁-C₆ alkyl-SOR²⁹, C₁-C₆ alkyl-SO₂R²⁹, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²⁴ is selected from —H, OH, C₁-C₁₀ alkyl, C₂-C₁₀ alkenyl, C₂-C₁₀ alkynyl, C₁-C₁₀ alkyl-R²⁹, C₂-C₁₀ alkenyl-R²⁹, C₂-C₁₀ alkynyl-R²⁹, C₁-C₁₀ alkyl-(R²⁹)₂, C₂-C₁₀ alkenyl-(R²⁹)₂, CSR²⁹, amino, NHR²⁵, NR²⁶R²⁶, N(R²⁵)—N(R²⁶)(R²⁶), C(R²⁹)═N-N(R²⁶)(R²⁶), N═N(R²⁵), N(R²⁵)—N═C(R²⁶), C(R²⁹)═N—O(R²⁷), ON═C(R²⁹), C₁-C₁₀ alkyl-NHR²⁵, C₁-C₁₀ alkyl-NR²⁶R²⁶, (C₁-C₁₀)alkyl-N(R²⁵)—N(R²⁶) (R²⁶), (C₁-C₁₀)alkylC(R²⁹)═N—N(R²⁶)(R²⁶), (C₁-C₁₀)alkyl-N═N(R²⁵), (C₁-C₁₀)alkyl-N(R²⁵)—N═C(R²⁶), SCN, NCS, C₁-C₁₀ alkyl SCN, C₁-C₁₀ alkyl NCS, nitro, cyano, O—R²⁷, C₁-C₁₀ alkyl-OR²⁷, COR²⁹, SR²⁷, SSR²⁷, SOR²⁹, SO₂R²⁹, C₁-C₁₀ alkyl-COR²⁹, C₁-C₁₀ alkyl-SR²⁷, C₁-C₁₀ alkyl-SOR²⁹, C₁-C₁₀ alkyl-SO₂R²⁹, halo, Si(R²⁹)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁰;
R²⁵ and R²⁶ are each independently selected from —H, C₁-C₆ alkyl, C₂-C₆ alkenyl, C₂-C₆ alkynyl, C₁-C₄ alkyl-R³⁵, C₁-C₆ alkyl-NHR³¹, C₁-C₆ alkyl-NR³¹R³², O—R³³, C₁-C₄ alkyl-OR³³, CO₂R³³, C(S)OR³³, C(O)SR³³, C(O)R³⁵, C(S)R³⁵, CONHR³⁴, C(S)NHR³⁴, CON(R³⁴)₂, C(S)N(R³⁴)₂, SR³³, SOR³⁵, SO₂R³⁵, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R²⁷ and R²⁸ are independently selected from —H, C₁-C₆ alkyl, C₂-C₆ alkenyl, C₂-C₆ alkynyl, C₁-C₆ alkyl-NHR³¹, C₁-C₆ alkyl-NR³¹R³², C₁-C₄ alkyl-OR³³, CSR¹¹, CO₂R³⁴, COR³⁵, CONHR³⁴, CON(R³⁴)₂, SOR³⁵, SO₂R³⁵, C₁-C₆ alkyl-CO₂R³⁴, C₁-C₆ alkyl-COR³⁵, C₁-C₆ alkyl-CONHR³⁴, C₁-C₆ alkyl-CON(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R²⁹ is selected from —H, C₁-C₆ alkyl, C₂-C₆ alkenyl, C₂-C₆ alkenyl-R³¹, C₁-C₆ alkyl-R³¹, C₂-C₆ alkynyl, amino, NHR³¹, NR³¹R³², C₁-C₆ alkyl-NHR³¹, C₁-C₆ alkyl-NR³¹R³², O—R³³, C₁-C₄ alkyl-OR³³, SR³³, C₁-C₆ alkyl-CO₂R³³, C₁-C₆ alkyl-C(S)OR³³, C₁-C₆ alkyl-C(O)SR³³, C₁-C₆ alkyl-COR³⁵, C₁-C₆ alkyl-C(S)R³⁵, C₁-C₆ alkyl-CONHR³⁴, C₁-C₆ alkyl-C(S)NHR³⁴, C₁-C₆ alkyl-CON(R³⁴)₂, C₁-C₆ alkyl-C(S)N(R³⁴)₂, C₁-C₆ alkyl-SR³³, C₁-C₆ alkyl-SOR³⁵, C₁-C₆ alkyl-SO₂R³⁵, halo C₁-C₄ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³⁰ is selected from —H, OH, C₁-C₁₀ alkyl, C₂-C₁₀ alkenyl, C₂-C₁₀ alkynyl, C₁-C₁₀ alkyl-R³⁵, C₂-C₁₀ alkenyl-R³⁵, C₂-C₁₀ alkynyl-R³⁵, C₁-C₁₀ alkyl-(R³⁵)₂, C₂-C₁₀ alkenyl-(R³⁵)₂, CSR³⁵, amino, NHR³¹, NR³²R³², N(R³¹)—N(R³²)(R³²), C(R³⁵)═N—N(R³²)(R³²), N═N(R³¹), N(R³¹)—N═C(R³²), C(R³⁵)═N—O(R³³), ON═C(R³⁵), C₁-C₁₀ alkyl-NHR³¹, C₁-C₁₀ alkyl-NR³²R³², (C₁-C₁₀)alkyl-N(R³¹)—N(R³²)(R³²), (C₁-C₁₀)alkylC(R³⁵)═N—N(R³²)(R³²), (C₁-C₁₀)alkyl-N═N(R³¹), (C₁-C₁₀)alkyl-N(R³¹)—N═C(R³²), SCN, NCS, C₁-C₁₀ alkyl SCN, C₁-C₁₀ alkyl NCS, nitro, cyano, O—R³³, C₁-C₁₀ alkyl-OR³³, COR³⁵, SR³³, SSR³³, SOR³⁵, SO₂R³⁵, C₁-C₁₀ alkyl-COR³⁵, C₁-C₁₀ alkyl-SR³³, C₁-C₁₀ alkyl-SOR³⁵, C₁-C₁₀ alkyl-SO₂R³⁵, halo, Si(R³⁵)₃, halo C₁-C₁₀ alkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³¹, R³², R³³ and R³⁴ are each independently selected from —H, alkyl, alkenyl, alkynyl, aminoalkyl, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³⁵ is selected from —H, alkyl, alkenyl, alkynyl, aminoalkyl, OH, alkoxy, amino, alkylamino, dialkylamino, hydroxyalkyl, alkylamino alkyl, dialkylaminoalkyl, alkoxyalkyl, aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl, wherein aryl, heteroaryl, heterocyclyl, alkylaryl, alkylheterocyclyl, alkylheteroaryl, arylalkyl, heteroarylalkyl, heterocyclylalkyl, and C₁-C₁₀ mono- and bicyclic cycloalkyl are optionally substituted with one or more of the groups defined by R³⁶;
R³⁶ is selected from —H, alkyl, alkenyl, alkynyl, aminoalkyl, OH, alkoxy, amino, nitro, cyano, halo, alkylamino, dialkylamino, hydroxyalkyl, alkylamino alkyl, dialkylaminoalkyl, alkoxyalkyl, aryl, heteroaryl, heterocyclyl, cycloalkyl, alkylaryl, alkylheterocyclyl, alkylheteroaryl, arylalkyl, heterocyclylalkyl, and heteroarylalkyl;
R², R⁵, R³⁸, R⁵⁰, R⁵¹, R⁵², R⁵³, and R⁵⁶ are each independently absent, or selected from an R^{b} component; and
R⁵⁴ and R⁵⁵ are each independently oxo, or absent; or
any two of R^{b}, R², R⁵, R⁵⁰, R⁵¹, R⁵², R⁵³, R⁵⁴, and R⁵⁶ optionally join to form a ring of 5, 6, 7, or 8 atoms, where the atoms in the ring are independently selected from M¹, M², M³, M⁴, M⁵, M⁶, Q¹, Q², Q³, Q⁴, Q⁵, Z¹, Z², Z³, Z⁴, Z⁵, CR³⁸, C(R³⁸)₂, C═O, NR⁷, O, S, C═S, S═O, and SO₂.

2. The compound according to claim 1, wherein:
p is 1;
T is N;
X is C;
R⁵⁴ is oxo; and
R⁵⁵ is absent.

3. The compound according to claim 1, wherein:
Z¹, Z², Z³, Z⁴, and Z⁵ form a pyrrole or imidazole ring.

4. The compound according to claim 1, wherein:
p is 1;
T is N;
X is C;
R⁵⁴ is oxo;
R⁵⁵ is absent; and
Z¹, Z², Z³, Z⁴, and Z⁵ form a pyrrole or imidazole ring.

5. The compound according to claim 4, wherein Z¹, Z², Z³, Z⁴, and Z⁵ form a pyrrole ring.

6. The compound according to claim 1, wherein:
p is 1;
T is N;
X is C;
R⁵⁴ is oxo;
R⁵⁵ is absent;
Z¹, Z², Z³, Z⁴, and Z⁵ form a pyrrole ring; and
R^{a} is

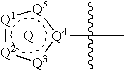

7. The compound according to claim 1, wherein:
p is 1;
T is N;
X is C;
R⁵⁴ is oxo;
R⁵⁵ is absent;
Z¹, Z², Z³, Z⁴, and Z⁵ form a pyrrole ring; and
R^{a} is

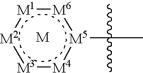

8. The compound according to claim 1, wherein:
p is 1 ;
T is N;
X is C;
R⁵⁴ is oxo;
R⁵⁵ is absent;
Z¹, Z², Z³, Z⁴, and Z⁵ form a pyrrole ring;
R^{a} is 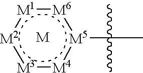
and, wherein the M-ring is selected from pyridine and pyrimidine.

9. The compound according to claim 8, wherein the M-ring is pyridine.

10. The compound according to claim 1, wherein:
p is 1;
T is N;
X is C;
Z¹, Z³, Z⁴, and Z⁵ are carbon;
Z² is nitrogen;
Z¹, Z², Z³, Z⁴ and Z⁵ form a pyrrole ring;
R^{a} is 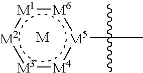
when ring M is aromatic, M² is N, M⁵ is carbon, M¹ is CR^{b}, M³ is CR⁵⁸, M⁴ is CR⁵⁹, and M⁶ is N, or CR⁶⁰;
when ring M is partially saturated, M² is N, M⁵ is carbon, M¹ is CR^{b} or C(R^{b})₂, M³ is CR⁵⁸ or C(R⁵⁸ )₂, M⁴ is CR⁵⁹ or C(R⁵⁹)₂, and M⁶ is independently selected from CR⁶⁰, N and C(R⁶⁰)₂;
M¹, M², M³, M⁴, M⁵ and M⁶ join to form a pyridine or pyrimidine ring;
R² is selected from H, and C₁-C₄ alkyl, or optionally is absent;
R⁵ is selected from H, halo, C₁-C₄ alkyl, amino, diazo, nitro, and aryl;
R⁵⁰ and R⁵¹ are each independently selected from H, C₁-C₄ alkyl, and aryl, or one of R⁵⁰ and R⁵¹ is absent;
R⁵² is selected from H, C₁-C₄ alkyl, C₁-C₄ haloalkyl, hydroxy C₁-C₄ alkyl, C₁-C₆ cycloalkyl, aryl, and aryl-C₁-C₄-alkoxy-C₁-C₄-alkyl;
R⁵³ is selected from H, C₁-C₄ alkenylcarboxyl, and C₁-C₄ alkyl;
R⁵⁴ is oxo;
R⁵⁵ is absent;
R⁵⁶ is absent, or is selected from an R⁵² group;
R⁵⁸ is selected from H, halo, amino, aryl-C₁-C₄-cycloalkyl, and haloaryl;
R⁵⁹ is selected from H, and halo, or optionally is absent, or R⁵⁷ and R⁵⁹ optionally join to form a six-membered phenyl ring; and
R⁶⁰ is H.

11. The compound according to claim 1, wherein:
p is 1;
T is N;
X is C;
Z¹, Z³, Z⁴, and Z⁵ are carbon;
Z² is nitrogen;
Z¹, Z², Z³, Z⁴ and Z⁵ form a pyrrole ring;
R^{a} is 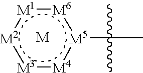
when ring M is aromatic, M² is N, M⁵ is carbon, M¹ is CR^{b}, M³ is CR⁵⁸, M⁴ is CR⁵⁹, and M⁶ is CR⁶⁰;
when ring M is partially saturated, M² is N, M⁵ is carbon, M¹ is CR^{b} or C(R^{b})₂, M³ is CR⁵⁵ or C(R⁵⁸)₂, M⁴ is CR⁵⁹ or C(R⁵⁹)₂, and M⁶ is independently selected from CR⁶⁰, and C(R⁶⁰)₂;
M¹, M², M³, M⁴, M⁵ and M⁶ join to form a pyridine ring;
R² is selected from H, and C₁-C₄ alkyl, or optionally is absent;
R⁵ is selected from H, halo, C₁-C₄ alkyl, amino, diazo, nitro, and aryl;
R⁵⁰ and R⁵¹ are each independently selected from H, C₁-C₄ alkyl, and aryl, or one of R⁵⁰ and R⁵¹ is absent;
R⁵² is selected from H, C₁-C₄ alkyl, C₁-C₄ haloalkyl, hydroxy C₁-C₄ alkyl, C₁-C₆ cycloalkyl, aryl, and aryl-C₁-C₄-alkoxy-C₁-C₄-alkyl;
R⁵³is selected from H, C₁-C₄ alkenylcarboxyl, and C₁-C₄ alkyl;
R⁵⁴ is oxo;
R⁵⁵ is absent;
R⁵⁶ is absent, or is selected from an R⁵² group;
R⁵⁸ is selected from H, halo, amino, aryl-C₁-C₄-cycloalkyl, and haloaryl;
R⁵⁹ is selected from H, and halo, or optionally is absent, or R⁵⁷ and R⁵⁹ optionally join to form a six-membered phenyl ring; and
R⁶⁰ is H.

12. The compound according to claim 1, wherein the compound comprises an irreversible inhibitor of MK-2.

13. The compound according to claim 12, wherein the compound comprises N-[3-[4-(4-oxo-4,5,6,7-tetrahydro-1H-pyrrolo[3,2-c]pyridin-2-yl)pyridin-2-yl]phenyl}acrylamide.

14. An MK-2 inhibiting compound that is selected from the MK-2 inhibiting compounds listed in Table I or Table II.

15. The compound according to claim 14, wherein the compound is 2-[(1E)-3-(3-fluorophenyl)-3-oxoprop-1-enyl]-1,5,6,7-tetrahydro-4H-pyrrolo[3,2-c]pyridin-4-one, or (4E)-4-[(3-fluorophenyl)hydrozono]-4-(4-oxo-4,5,6,7-tetrahydro-1H-pyrrolo[3,2-c]pyridin-2-yl)butanoic acid.

16. The compound according to claim 14, wherein the compound is selected from the group consisting of:
4-(4-oxo-4,5,6,7-tetrahydro-1H-pyrrolo[3,2-c]pyridin-2-yl)pyridine-2-carbaldehyde methyl[4-(morpholin-4-ylcarbonyl)phenyl]hydrazone trifluoroacetate,
4-(4-oxo-4,5,6,7-tetrahydro-1H-pyrrolo[3,2-c]pyridin-2-yl)pyridine-2-carbaldehyde [4-(pyrrolidin-1-ylcarbonyl)phenyl]hydrazone,
2-bromo-N-{4-[4-(4-oxo-4,5,6,7-tetrahydro-1H-pyrrolo[3,2-c]pyridin-2-yl)pyridin-2-yl]phenyl}acetamide trifluoroacetate,
2-(5-fluoro-2-quinolin-3-ylpyridin-4-yl)-1,5,6,7-tetrahydro-4H-pyrrolo[3,2-c]pyridin-4-one trifluoroacetate,
4-{[2-(pyrrolidin-1-ylmethyl) pyrrolidin-1-yl]carbonyl}benzaldehyde [4-(4-oxo-4,5,6,7-tetrahydro-1H-pyrrolo[3,2-c]pyridin-2-yl) pyridin-2-yl]hydrazone bis(trifluoroacetate),
2-(2-quinolin-3-ylpyrimidin-4-yl)-1,5,6,7-tetrahydro-4H-pyrrolo[3,2-c]pyridin-4-one,
N-cyclopentyl-4-[4-(4-oxo-4,5,6,7-tetrahydro-1H-pyrrolo[3,2-c]pyridin-2-yl)pyridin-2-yl]benzamide,
2-{2-[(E)-2-phenylethenyl]pyridin-4-yl}-1,5,6,7-tetrahydro-4H-pyrrolo[3,2-c]pyridin-4-one,
N-benzyl-4-[4-(4-oxo-4,5,6,7-tetrahydro-1H-pyrrolo[3,2-c]pyridin-2-yl)pyridin-2-yl]benzamide trifluoroacetate,
2-(2-quinolin-3-ylpyridin-4-yl)-1,5,6,7-tetrahydro-4H-pyrrolo[3,2-c]pyridin-4-one trifluoroacetate,
N-{4-[4-(4-oxo-4,5,6,7-tetrahydro-1H-pyrrolo[3,2-c]pyridin-2-yl)pyridin-2-yl]phenyl}-2-pyridin-4-ylacetamide bis(trifluoroacetate),
2-(4-fluorophenyl)-N-{3-[4-(4-oxo-4,5,6,7-tetrahydro-1H-pyrrolo[3,2-c]pyridin-2-yl)pyridin-2-yl]phenyl}acetamide trifluoroacetate,
N-cyclopentyl-3-[4-(4-oxo-4,5,6,7-tetrahydro-1H-pyrrolo[3,2-c]pyridin-2-yl)pyridin-2-yl]benzamide trifluoroacetate,
2-(2-{(E)-2-[4-(morpholin-4-ylmethyl)phenyl]vinyl}pyridin-4-yl)-1,5,6,7-tetrahydro-4H-pyrrolo[3,2-c]pyridin-4-one trifluoroacetate,
4-(4-oxo-4,5,6,7-tetrahydro-1H-pyrrolo[3,2-c]pyridin-2-yl)pyridine-2-carbaldehyde [4-(morpholin-4-ylcarbonyl)phenyl]hydrazone,
4-(4-oxo-4,5,6,7-tetrahydro-1H-pyrrolo[3,2-c]pyridin-2-yl)pyridine-2-carbaldehyde [4-(methylsulfonyl)phenyl]hydrazone,
2-[2-(6-hydroxy-2-naphthyl)pyridin-4-yl]-1,5,6,7-tetrahydro-4H-pyrrolo[3,2-c]pyridin-4-one trifluoroacetate,
2-(2-{(E)-2-[4-(morpholin-4-ylcarbonyl)phenyl]vinyl}pyridin-4-yl)-1,5,6,7-tetrahydro-4H-pyrrolo[3,2-c]pyridin-4-one trifluoroacetate,
2-{2-[(E)-2-(2-fluoro-4-morpholin-4-ylphenyl)vinyl]pyridin-4-yl}-1,5,6,7-tetrahydro-4H-pyrrolo[3,2-c]pyridin-4-one trifluoroacetate,
2-{2-[(E)-2-(4-morpholin-4-ylphenyl)vinyl]pyridin-4-yl}-1,5,6,7-tetrahydro-4H-pyrrolo[3,2-c]pyridin-4-one trifluoroacetate,
2-{2-[(E)-2-(4-fluorophenyl)ethenyl]pyridin-4-yl}-1,5,6,7-tetrahydro-4H-pyrrolo[3,2-c]pyridin-4-one,
2-{2-[(E)-2-(2-chlorophenyl)vinyl]pyridin-4-yl}-1,5,6,7-tetrahydro-4H-pyrrolo[3,2-c]pyridin-4-one trifluoroacetate,
benzaldehyde [4-(4-oxo-4,5,6,7-tetrahydro-1H-pyrrolo[3,2-c]pyridin-2-yl)pyridin-2-yl]hydrazone,
2-chloro-N-{4-[4-(4-oxo-4,5,6,7-tetrahydro-1H-pyrrolo[3,2-c]pyridin-2-yl)pyridin-2-yl]phenyl}acetamide trifluoroacetate, and
(2E)-4-bromo-N-{4-[4-(4-oxo-4,5,6,7-tetrahydro-1H-pyrrolo[3,2-c]pyridin-2-yl)pyridin-2-yl]phenyl}but-2-enamide trifluoroacetate.

17. A method of inhibiting MK-2, the method comprising contacting MK-2 with at least one compound having the structure described in claim 1.

18. A method of inhibiting MK-2, the method comprising contacting MK-2 with at least one compound that is selected from the compounds described in claim 14.

19. The method according to claim 17, wherein the MK-2 inhibitory compound is an irreversible inhibitor of MK-2.

20. The method according to claim 19, wherein the irreversible inhibitor comprises N-[3-[4-(4-oxo-4,5,6,7-tetrahydro-1H-pyrrolo[3,2-c]pyridin-2-yl)pyridin-2-yl]phenyl}acrylamide.

21. A method of preventing or treating a TNFα mediated disease or disorder in a subject, the method comprising administering to the subject an effective amount of an MK-2 inhibiting compound having the structure described in claim 1.

22. The method according to claim 21, wherein the subject is one that is in need of such prevention or treatment.

23. The method according to claim 21, wherein the subject is a mammal.

24. The method according to claim 21, wherein the subject is a human.

25. The method according to claim 21, wherein the TNFα mediated disease or disorder is selected from the group consisting of connective tissue and joint disorders, neoplasia disorders, cardiovascular disorders, otic disorders, ophthalmic disorders, respiratory disorders, gastrointestinal disorders, angiogenesis-related disorders, immunological disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

26. The method according to claim 21, wherein the TNFα mediated disease or disorder is selected from the group consisting of: arthritis, rheumatoid arthritis, spondyloarthopathies, gouty arthritis, osteoarthritis, systemic lupus erythematosus, juvenile arthritis, asthma, bronchitis, menstrual cramps, tendinitis, bursitis, connective tissue injuries or disorders, skin related conditions, psoriasis, eczema, burns, dermatitis, gastrointestinal conditions, inflammatory bowel disease, gastric ulcer, gastric varices, Crohn's disease, gastritis, irritable bowel syndrome, ulcerative colitis, cancer, colorectal cancer, herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, ophthalmic diseases, retinitis, retinopathies, conjunctivitis, uveitis, ocular photophobia, acute injury to the eye tissue, pulmonary inflammation, viral infections, cystic fibrosis, central nervous system disorders, cortical dementias, and Alzheimer's disease.

27. A method of preventing or treating a TNFα mediated disease or disorder in a subject, the method comprising administering to the subject at least one MK-2 inhibiting compound that is selected from the group consisting of the compounds described in claim 14.

28. A therapeutic composition comprising a compound having the structure described in claim 1.

29. A therapeutic composition comprising at least one MK-2 inhibitory compound that is described in claim 14.

30. A pharmaceutical composition comprising a pharmaceutically acceptable carrier and at least one MK-2 inhibitory compound having the structure described in claim 1.

31. The pharmaceutical composition according to claim 28, wherein the MK-2 inhibitory compound has an IC₅₀ for MK-2 of not over 0.1 mM.

32. The pharmaceutical composition according to claim 28, where the compound is an irreversible inhibitor of MK-2.

33. The pharmaceutical composition according to claim 28, wherein the MK-2 inhibitory compound comprises N-[3-[4-(4-oxo-4,5,6,7-tetrahydro-1H-pyrrolo[3,2-c]pyridin-2-yl)pyridin-2-yl]phenyl}acrylamide.

34. A kit comprising a dosage form that includes a therapeutically effective amount of at least one MK-2 inhibitory compound having a structure described in claim 1.
